(19) United States
(12) Patent Application Publication
(10) Pub. No.: US 2024/0018161 A1
(43) Pub. Date: Jan. 18, 2024

(54) MODULATORS OF CYSTIC FIBROSIS TRANSMEMBRANE CONDUCTANCE REGULATOR

(71) Applicant: VERTEX PHARMACEUTICALS INCORPORATED, Boston, MA (US)

(72) Inventors: Jason MCCARTNEY, Cardiff by the Sea, CA (US); Alexander Russell ABELA, Escondido, CA (US); Sunny ABRAHAM, San Diego, CA (US); Corey Don ANDERSON, Brighton, MA (US); Vijayalaksmi ARUMUGAM, San Marcos, CA (US); Jaclyn CHAU, San Diego, CA (US); Jeremy CLEMENS, San Diego, CA (US); Thomas CLEVELAND, San Marcos, CA (US); Timothy Richard COON, Carlsbad, CA (US); Andrew DINH, La Mesa, CA (US); Timothy A. DWIGHT, Quincy, MA (US); Lev Tyler Dewey FANNING, San Marcos, CA (US); Bryan A. FRIEMAN, La Jolla, CA (US); Peter GROOTENHUIS, Del Mar, CA (US); Sara Sabina HADIDA RUAH, La Jolla, CA (US); Yoshihiro ISHIHARA, San Diego, CA (US); Paul KRENITSKY, San Francisco, CA (US); Mark Thomas MILLER, Rancho Santa Fe, CA (US); Fabrice PIERRE, La Jolla, CA (US); Alina SILINA, Needham, MA (US); Joe A. TRAN, San Marcos, CA (US); Lino VALDEZ, San Diego, CA (US); Jinglan ZHOU, San Diego, CA (US)

(21) Appl. No.: 18/030,530

(22) PCT Filed: Oct. 6, 2021
(86) PCT No.: PCT/US2021/053861

§ 371 Date: Apr. 6, 2023

Related U.S. Application Data
(60) Provisional application No. 63/088,799, filed on Oct. 7, 2020.

(51) Int. Cl. C07D 515/04; C07D 515/14; A61K 45/06

(52) U.S. Cl. CPC C07D 515/04; A61K 45/06; C07D 515/14

(57) ABSTRACT

This disclosure provides modulators of Cystic Fibrosis Transmembrane Conductance Regulator (CFTR) having core structure (I), pharmaceutical compositions containing at least one such modulator, methods of treatment of CFTR mediated diseases, including cystic fibrosis, using such modulators and pharmaceutical compositions, combination pharmaceutical compositions and combination therapies, and processes and intermediates for making such modulators.

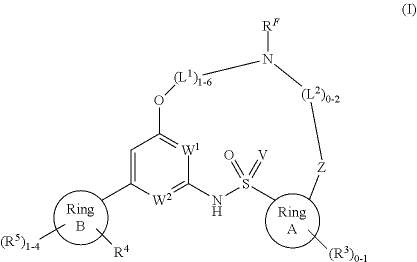

This application claims the benefit of priority of U.S. Provisional Application No. 63/088,799, filed Oct. 7, 2020, the contents of which are incorporated by reference herein in their entirety.

The disclosure relates to modulators of Cystic Fibrosis Transmembrane Conductance Regulator (CFTR), pharmaceutical compositions containing the modulators, methods of treatment of CFTR mediated diseases, including cystic fibrosis, using such modulators, combination therapies and combination pharmaceutical compositions employing such modulators, and processes and intermediates for making such modulators.

Cystic fibrosis (CF) is a recessive genetic disease that affects approximately 70,000 children and adults worldwide. Despite progress in the treatment of CF, there is no cure.

In patients with CF, mutations in CFTR endogenously expressed in respiratory epithelia lead to reduced apical anion secretion causing an imbalance in ion and fluid transport. The resulting decrease in anion transport contributes to increased mucus accumulation in the lung and accompanying microbial infections that ultimately cause death in CF patients. In addition to respiratory disease, CF patients typically suffer from gastrointestinal problems and pancreatic insufficiency that, if left untreated, result in death. In addition, the majority of males with cystic fibrosis are infertile, and fertility is reduced among females with cystic fibrosis.

Sequence analysis of the CFTR gene has revealed a variety of disease-causing mutations (Cutting, G. R. et al. (1990) Nature 346:366-369; Dean, M. et al. (1990) Cell 61:863:870; and Kerem, B-S. et al. (1989) Science 245:1073-1080; Kerem, B-S et al. (1990) Proc. Natl. Acad. Sci. USA 87:8447-8451). To date, greater than 2000 mutations in the CF gene have been identified; currently, the CFTR2 database contains information on only 432 of these identified mutations, with sufficient evidence to define 352 mutations as disease-causing. The most prevalent disease-causing mutation is a deletion of phenylalanine at position 508 of the CFTR amino acid sequence and is commonly referred to as the F508del mutation. This mutation occurs in many of the cases of cystic fibrosis and is associated with severe disease.

The deletion of residue 508 in CFTR prevents the nascent protein from folding correctly. This results in the inability of the mutant protein to exit the endoplasmic reticulum (ER) and traffic to the plasma membrane. As a result, the number of CFTR channels for anion transport present in the membrane is far less than observed in cells expressing wild-type CFTR, i.e., CFTR having no mutations. In addition to impaired trafficking, the mutation results in defective channel gating. Together, the reduced number of channels in the membrane and the defective gating lead to reduced anion and fluid transport across epithelia. (Quinton, P. M. (1990), FASEB J. 4: 2709-2727). The channels that are defective because of the F508del mutation are still functional, albeit less functional than wild-type CFTR channels. (Dalemans et al. (1991), Nature Lond. 354: 526-528; Pasyk and Foskett (1995), J. Cell. Biochem. 270: 12347-50). In addition to F508del, other disease-causing mutations in CFTR that result in defective trafficking, synthesis, and/or channel gating could be up- or down-regulated to alter anion secretion and modify disease progression and/or severity.

CFTR is a cAMP/ATP-mediated anion channel that is expressed in a variety of cell types, including absorptive and secretory epithelia cells, where it regulates anion flux across the membrane, as well as the activity of other ion channels and proteins. In epithelial cells, normal functioning of CFTR is critical for the maintenance of electrolyte transport throughout the body, including respiratory and digestive tissue. CFTR is composed of 1480 amino acids that encode a protein which is made up of a tandem repeat of transmembrane domains, each containing six transmembrane helices and a nucleotide binding domain. The two transmembrane domains are linked by a large, polar, regulatory (R)-domain with multiple phosphorylation sites that regulate channel activity and cellular trafficking.

Chloride transport takes place by the coordinated activity of ENaC and CFTR present on the apical membrane and the Na⁺—K⁺-ATPase pump and Cl— channels expressed on the basolateral surface of the cell. Secondary active transport of chloride from the luminal side leads to the accumulation of intracellular chloride, which can then passively leave the cell via Cl⁻ channels, resulting in a vectorial transport. Arrangement of Na⁺/2Cl⁻/K⁺ co-transporter, Na⁺—K⁺-ATPase pump and the basolateral membrane K⁺ channels on the basolateral surface and CFTR on the luminal side coordinate the secretion of chloride via CFTR on the luminal side. Because water is probably never actively transported itself, its flow across epithelia depends on tiny transepithelial osmotic gradients generated by the bulk flow of sodium and chloride.

A number of CFTR modulating compounds have recently been identified. However, compounds that can treat or reduce the severity of cystic fibrosis and other CFTR mediated diseases, and particularly the more severe forms of these diseases, are still needed.

One aspect of the disclosure provides novel compounds, including compounds of Formula I, compounds of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing.

Formula I encompasses compounds falling within the following structure:

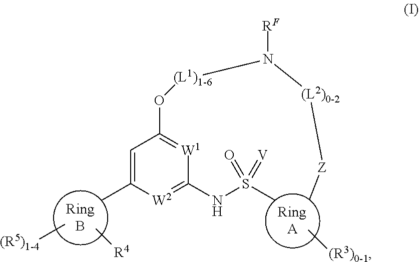

and includes tautomers of those compounds, deuterated derivatives of any of the compounds and tautomers, and pharmaceutically acceptable salts of any of the foregoing, wherein:
Ring A is selected from:
   C₆-C₁₀ aryl,
   C₃-C₁₀ cycloalkyl,
   3- to 10-membered heterocyclyl, and
   5- to 10-membered heteroaryl;
Ring B is selected from:
   C₆-C₁₀ aryl,
   C₃-C₁₀ cycloalkyl,
   3- to 10-membered heterocyclyl, and
   5- to 10-membered heteroaryl;
V is selected from O and NH;
W¹ is selected from N and CH;
W² is selected from N and CH; provided that at least one of W¹ and W² is N;
Z is selected from O, NR^{ZN}, and C(R^{ZC})₂, provided that when L² is absent, Z is C(R^{ZC})₂;
each L¹ is independently selected from C(R^{L1})₂;
each L² is independently selected from C(R^{L2})₂;
each R³ is independently selected from:
   halogen,
   C₁-C₆ alkyl,
   C₁-C₆ alkoxy,
   C₃-C₁₀ cycloalkyl,
   C₆-C₁₀ aryl optionally substituted with 1-3 groups independently selected from C₁-C₆ alkyl, and
   3- to 10-membered heterocyclyl;
R⁴ is selected from hydrogen and C₁-C₆ alkyl;
each R⁵ is independently selected from:
   hydrogen,
   halogen,
   hydroxyl,
   N(RN)₂,
   —SO-Me,
   —CH═C(R^{L}c)₂, wherein both R^{LC} are taken together to form a C₃-C₁₀ cycloalkyl,
   C₁-C₆ alkyl optionally substituted with 1-3 groups independently selected from:
      hydroxyl,
      C₁-C₆ alkoxy optionally substituted with 1-3 groups independently selected from C₁-C₆ alkoxy and C₆-C₁₀ aryl,
      C₃-C₁₀ cycloalkyl,
      —(O)₀₋₁—(C₆-C₁₀ aryl) optionally substituted with 1-3 groups independently selected from C₁-C₆ alkyl and C₁-C₆ alkoxy,
      3- to 10-membered heterocyclyl, and
      N(RN)₂,
   C₁-C₆ alkoxy optionally substituted with 1-3 groups independently selected from:
      halogen,
      C₆-C₁₀ aryl, and
      C₃-C₁₀ cycloalkyl optionally substituted with 1-3 groups independently selected from C₁-C₆ fluoroalkyl,
   C₁-C₆ fluoroalkyl,
   C₃-C₁₀ cycloalkyl,
   C₆-C₁₀ aryl, and
   3- to 10-membered heterocyclyl;
R^{ZN} is selected from:
   hydrogen,
   C₁-C₉ alkyl optionally substituted with 1-3 groups independently selected from:
      hydroxyl,
      oxo,
      cyano,
      C₁-C₆ alkoxy optionally substituted with 1-3 groups independently selected from halogen and C₁-C₆ alkoxy,
      N(RN)₂,
      SO₂Me,
      C₃-C₁₀ cycloalkyl optionally substituted with 1-3 groups independently selected from:
         hydroxyl,
         C₁-C₆ alkyl optionally substituted with 1-3 groups independently selected from hydroxyl, oxo, C₁-C₆ alkoxy, C₆-C₁₀ aryl, and N(R^{N})₂,
         C₁-C₆ fluoroalkyl,
         C₁-C₆ alkoxy, and
         COOH,
         N(RN)₂,
         C₆-C₁₀ aryl, and
         3- to 10-membered heterocyclyl optionally substituted with 1-3 groups independently selected from oxo and C₁-C₆ alkyl,
      C₆-C₁₀ aryl optionally substituted with 1-3 groups independently selected from:
         halogen,
         hydroxyl,
         cyano,
         SiMe₃,
         SO₂Me,
         SF₅,
         N(RN)₂,
         P(O)Me₂,
         —(O)₀₋₁—(C₃-C₁₀ cycloalkyl) optionally substituted with 1-3 groups independently selected from C₁-C₆ fluoroalkyl,
         C₁-C₆ alkyl optionally substituted with 1-3 groups independently selected from hydroxyl, oxo, C₁-C₆ alkoxy, 5- to 10-membered heteroaryl, SO₂Me, and N(RN)₂,
         C₁-C₆ alkoxy optionally substituted with 1-3 groups independently selected from hydroxyl, oxo, N(R^{N})₂, and C₆-C₁₀ aryl,
         C₁-C₆ fluoroalkyl,
         3- to 10-membered heterocyclyl optionally substituted with 1-3 groups independently selected from C₁-C₆ alkyl,
         —(O)₀₋₁—(C₆-C₁₀ aryl), and
         —(O)₀₋₁-(5- to 10-heteroaryl) optionally substituted with hydroxyl, oxo, N(R^{N})₂, C₁-C₆ alkyl, C₁-C₆ alkoxy, C₁-C₆ fluoroalkyl, and C₃-C₁₀ cycloalkyl,
      3- to 10-membered heterocyclyl optionally substituted with 1-4 groups independently selected from:
         hydroxyl,
         oxo,
         N(RN)₂,
         C₁-C₆ alkyl (optionally substituted with 1-3 groups independently selected from oxo and C₁-C₆ alkoxy),
         C₁-C₆ alkoxy,
         C₁-C₆ fluoroalkyl,
         C₆-C₁₀ aryl optionally substituted with 1-3 groups independently selected from halogen, and
         5- to 10-membered heteroaryl, and
      5- to 10-membered heteroaryl optionally substituted with 1-3 groups independently selected from:
         hydroxyl,
         cyano,
         oxo,
         halogen,
         B(OH)₂,
         N(RN)₂,
         C₁-C₆ alkyl optionally substituted with 1-3 groups independently selected from hydroxyl, oxo, C₁-C₆ alkoxy (optionally substituted with 1-3-SiMe₃), and N(R^{N})₂,
         C₁-C₆ alkoxy optionally substituted with 1-3 groups independently selected from hydroxyl, oxo, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from C₁-C₆ alkyl,
         —(O)₀₋₁—(C₆-C₁₀ aryl),
         —(O)₀₋₁-(3- to 10-membered heterocyclyl) optionally substituted with 1-4 groups independently selected from hydroxyl, oxo, halogen, cyano, N(R^{N})₂, C₁-C₆ alkyl (optionally substituted with 1-3 groups independently selected from hydroxyl, oxo, N(R^{N})₂, and C₁-C₆ alkoxy), C₁-C₆ alkoxy, C₁-C₆ fluoroalkyl, 3- to 10-membered heterocyclyl (optionally substituted with 1-3 groups independently selected from C₁-C₆ fluoroalkyl) and
         5- to 10-membered heteroaryl optionally substituted with 1-4 groups independently selected from C₁-C₆ alkyl and C₃-C₁₀ cycloalkyl,
   C₁-C₆ fluoroalkyl,
   C₃-C₁₀ cycloalkyl optionally substituted with 1-3 groups independently selected from:
      hydroxyl,
      oxo,
      halogen,
      cyano,
      N(RN)₂,
      C₁-C₆ alkyl optionally substituted with 1-3 groups independently selected from:
         hydroxyl,
         oxo,
         N(RN)₂,
         C₁-C₆ alkoxy, and
         C₆-C₁₀ aryl,
      C₁-C₆ alkoxy optionally substituted with 1-3 groups independently selected from halogen, oxo, C₆-C₁₀ aryl, and N(R^{N})₂,
      halogen,
      C₃-C₁₀ cycloalkyl,
      3- to 10-member heterocyclyl optionally substituted with 1-3 groups independently selected from C₁-C₆ alkyl, and
      5- to 10-membered heteroaryl optionally substituted with 1-3 groups independently selected from:
         hydroxyl,
         cyano,
         oxo,
         halogen,
         N(RN)₂,
         C₁-C₆ alkyl optionally substituted with 1-3 groups independently selected from hydroxyl, oxo, C₁-C₆ alkoxy, and N(R^{N})₂,
         C₁-C₆ alkoxy optionally substituted with 1-3 groups independently selected from hydroxyl, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from C₁-C₆ alkyl,
         C₆-C₁₀ aryl, and
         3- to 10-membered heterocyclyl optionally substituted with 1-3 groups independently selected from C₁-C₆ alkyl,
   C₆-C₁₀ aryl,
   3- to 10-membered heterocyclyl optionally substituted with 1-3 groups independently selected from:
      oxo,
      C₁-C₆ alkyl optionally substituted with 1-3 groups independently selected from:
         oxo,
         hydroxyl,
         N(RN)₂,
         C₁-C₆ alkoxy optionally substituted with 1-3 groups independently selected from halogen and C₆-C₁₀ aryl, and
         —(O)₀₋₁—(C₃-C₁₀ cycloalkyl),
      C₁-C₆ fluoroalkyl,
      C₃-C₁₀ cycloalkyl optionally substituted with 1-3 groups independently selected from halogen, and
      3- to 10-membered heterocyclyl,
   5- to 10-membered heteroaryl optionally substituted with 1-3 groups independently selected from:
      halogen,
      C₁-C₆ alkyl optionally substituted with 1-3 groups independently selected from oxo, C₁-C₆ alkoxy, and N(R^{N})₂, and
      3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl),
   R^{F};
each R^{ZC} is independently selected from:
   hydrogen,
   C₁-C₆ alkyl optionally substituted with 1-3 groups independently selected from C₆-C₁₀ aryl (optionally substituted with 1-3 groups independently selected from C₁-C₆ alkyl),
   C₆-C₁₀ aryl optionally substituted with 1-3 groups independently selected from C₁-C₆ alkyl, and
   R^{F};
or two R^{ZC} are taken together to form an oxo group;
each R^{L1} is independently selected from:
   hydrogen,
   N(R^{N})₂, provided that two N(R^{N})₂ are not bonded to the same carbon,
   C₁-C₉ alkyl optionally substituted with 1-3 groups independently selected from:
      halogen,
      hydroxyl,
      oxo,
      N(RN)₂,
      C₁-C₆ alkoxy optionally substituted with 1-3 groups independently selected from C₆-C₁₀ aryl,
      C₃-C₁₀ cycloalkyl optionally substituted with 1-3 groups independently selected from halogen and C₁-C₆ fluoroalkyl,
      C₆-C₁₀ aryl optionally substituted with 1-3 groups independently selected from C₁-C₆ alkyl, and
      3- to 10-membered heterocyclyl optionally substituted with 1-3 groups independently selected from C₁-C₆ alkyl (optionally substituted with 1-3 groups independently selected from hydroxyl and oxo),
   C₃-C₁₀ cycloalkyl,
   C₆-C₁₀ aryl optionally substituted with 1-4 groups independently selected from:
      halogen,
      cyano,
      SiMe₃,
      POMe₂,
      C₁-C₇ alkyl optionally substituted with 1-3 groups independently selected from:
         hydroxyl,
         oxo,
         cyano,
         SiMe₃,
         N(RN)₂, and
         C₃-C₁₀ cycloalkyl optionally substituted with 1-3 groups independently selected from C₁-C₆ fluoroalkyl,
      C₁-C₆ alkoxy optionally substituted with 1-3 groups independently selected from:
         C₃-C₁₀ cycloalkyl optionally substituted with 1-3 groups independently selected from C₁-C₆ fluoroalkyl, and
         C₁-C₆ alkoxy,
      C₁-C₆ fluoroalkyl,
      C₃-C₁₀ cycloalkyl optionally substituted with 1-3 groups independently selected from C₁-C₆ alkyl and C₁-C₆ fluoroalkyl,
      C₆-C₁₀ aryl,
      3- to 10-membered heterocyclyl optionally substituted with 1-3 groups independently selected from C₁-C₆ alkyl, and
      5- to 10-membered heteroaryl,
   3- to 10-membered heterocyclyl optionally substituted with 1-3 groups independently selected from:
      C₁-C₆ alkyl optionally substituted with 1-3 groups independently selected from:
         oxo, and
         C₁-C₆ alkoxy,
   5- to 10-membered heteroaryl optionally substituted with 1-3 groups independently selected from:
      C₁-C₆ alkyl optionally substituted with 1-3 groups independently selected from:
         C₃-C₁₀ cycloalkyl optionally substituted with 1-3 groups independently selected from C₁-C₆ fluoroalkyl, and
      C₆-C₁₀ aryl optionally substituted with 1-3 groups independently selected from C₁-C₆ alkyl, and
   R^{F};
or two R^{L1} on the same carbon atom are taken together to form an oxo group;
each R^{L2} is independently selected from hydrogen and R^{F}; or two R^{L2} on the same carbon atom are taken together to form an oxo group; provided that at least one R^{L1} or R^{L2} is R^{F};
each R^{N} is independently selected from:
   hydrogen,
   C₁-C₈ alkyl optionally substituted with 1-3 groups independently selected from:
      oxo,
      halogen,
      hydroxyl,
      NH₂,
      NHMe,
      NMe₂,
      C₁-C₆ alkoxy optionally substituted with 1-3 groups independently selected from C₆-C₁₀ aryl,
      —(O)₀₋₁—(C₃-C₁₀ cycloalkyl),
      C₆-C₁₀ aryl optionally substituted with 1-3 groups independently selected from halogen and C₁-C₆ alkyl, and
      3- to 14-membered heterocyclyl optionally substituted with 1-4 groups independently selected from oxo and C₁-C₆ alkyl, and
      5- to 14-membered heteroaryl optionally substituted with 1-4 groups independently selected from oxo and C₁-C₆ alkyl,
   C₃-C₁₀ cycloalkyl optionally substituted with 1-3 groups independently selected from:
      hydroxyl,
      NH₂, and
      NHMe, and
      C₁-C₆ alkyl optionally substituted with 1-3 groups independently selected from hydroxyl,
   C₆-C₁₀ aryl, and
   3- to 10-membered heterocyclyl;
or two R^{N} on the same nitrogen atom are taken together with the nitrogen to which they are bonded to form a 3- to 10-membered heterocyclyl optionally substituted with 1-3 groups selected from:
   hydroxyl,
   oxo,
   cyano,
   C₁-C₆ alkyl optionally substituted with 1-3 groups independently selected from oxo, hydroxyl, C₁-C₆ alkoxy, and N(R^{N2})₂, wherein each R^{N2} is independently selected from hydrogen and C₁-C₆ alkyl,
   C₁-C₆ alkoxy, and
   C₁-C₆ fluoroalkyl;
or one R⁴ and one R^{L1} are taken together to form a C₆-C₈ alkylene;
two R^{F} taken together with the atoms to which they are bonded form a group selected from:
   C₃-C₁₀ cycloalkyl optionally substituted with 1-3 groups independently selected from C₁-C₆ alkyl,
   C₆-C₁₀ aryl optionally substituted with 1-3 groups independently selected from:
      halogen,
      C₁-C₆ alkyl,
      N(R^{N})₂, and
      3- to 10-membered heterocyclyl optionally substituted with 1-3 groups independently selected from hydroxyl,
   3- to 11-membered heterocyclyl optionally substituted with 1-3 groups independently selected from:
      oxo,
      N(RN)₂,
      C₁-C₉ alkyl optionally substituted with 1-4 groups independently selected from:
         oxo,
         halogen,
         hydroxyl,
         N(RN)₂,
         SO₂—(C₁-C₆ alkyl),
         C₁-C₆ alkoxy optionally substituted with 1-3 groups independently selected from halogen, C₆-C₁₀ aryl,
         C₆-C₁₀ aryl optionally substituted with 1-3 groups independently selected from hydroxyl, halogen, cyano, C₁-C₆ alkyl (optionally substituted with 1-3 groups independently selected from oxo and C₁-C₆ alkoxy), C₁-C₆ alkoxy (optionally substituted with 1-3 groups independently selected from C₆-C₁₀ aryl), —(O)₀₋₁—(C₁-C₆ fluoroalkyl), and C₆-C₁₀ aryl (optionally substituted with 1-3 groups independently selected from C₁-C₆ alkoxy),
         —(O)₀₋₁—(C₃-C₁₀ cycloalkyl) optionally substituted with 1-4 groups independently selected from hydroxyl, halogen, N(R^{N})₂, C₁-C₆ alkyl (optionally substituted with 1-3 groups independently selected from oxo, hydroxyl, and C₁-C₆ alkoxy), C₁-C₆ fluoroalkyl, and C₆-C₁₀ aryl,
         3- to 10-membered heterocyclyl optionally substituted with 1-3 groups independently selected from oxo, C₁-C₆ alkyl (optionally substituted with 1-3 groups independently selected from C₆-C₁₀ aryl (optionally substituted with 1-3 groups independently selected from halogens)), C₁-C₆ alkoxy, C₃-C₁₀ cycloalkyl, and R^{N},
         —O-(5- to 12-membered heteroaryl) optionally substituted with 1-3 groups independently selected from C₆-C₁₀ aryl (optionally substituted with 1-3 groups independently selected from halogen) and C₁-C₆ alkyl, and
         5- to 10-membered heteroaryl optionally substituted with 1-3 groups independently selected from hydroxyl, oxo, N(R^{N})₂, C₁-C₆ alkyl (optionally substituted with 1-3 groups independently selected from cyano), C₁-C₆ alkoxy, —(O)₀₋₁—(C₁-C₆ fluoroalkyl), —O—(C₆-C₁₀ aryl), and C₃-C₁₀ cycloalkyl,
      C₃-C₁₂ cycloalkyl optionally substituted with 1-4 groups independently selected from halogen, C₁-C₆ alkyl, and C₁-C₆ fluoroalkyl,
      C₆-C₁₀ aryl,
      3- to 10-membered heterocyclyl, and
      5- to 10-membered heteroaryl optionally substituted with 1-3 groups independently selected from C₁-C₆ alkoxy, C₁-C₆ fluoroalkyl, and N(R^{N})₂, and
   5- to 12-membered heteroaryl optionally substituted with 1-3 groups independently selected from C₁-C₆ alkyl and C₁-C₆ fluoroalkyl.

In some embodiments of Formula I, when two R^{F} taken together form a 3- to 11-membered heterocyclyl, wherein the 3- to 11-membered heterocyclyl is optionally substituted with a 5- to 10-membered heteroaryl, and wherein the 5- to 10-membered heteroaryl is optionally substituted with a C₁-C₆ alkoxy, the C₁-C₆ alkoxy may be optionally substituted with C₆-C₁₀ aryl.

Formula I also includes compounds of Formula Ia:

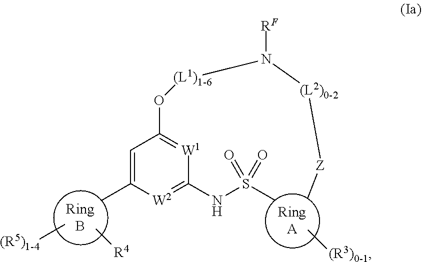

tautomers of those compounds, deuterated derivatives of any of the compounds and tautomers, and pharmaceutically acceptable salts of any of the foregoing, wherein Ring A, Ring B, W¹, W², Z, L¹, L², R³, R⁴, R⁵, and R^{F} are as defined for Formula I.

Formula I also includes compounds of Formula IIa:

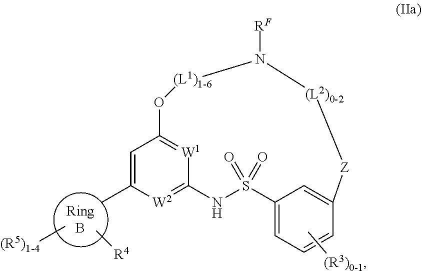

tautomers of those compounds, deuterated derivatives of any of the compounds and tautomers, and pharmaceutically acceptable salts of any of the foregoing, wherein Ring B, W¹, W², Z, L¹, L², R³, R⁴, R⁵, and R^{F} are as defined for Formula I.

Formula I also includes compounds of Formula IIb:

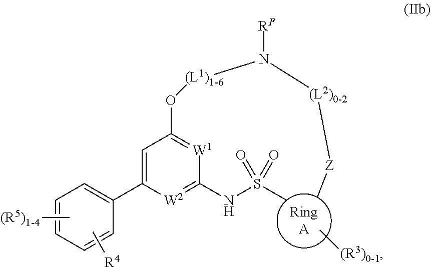

tautomers of those compounds, deuterated derivatives of any of the compounds and tautomers, and pharmaceutically acceptable salts of any of the foregoing, wherein Ring A, W¹, W², Z, L¹, L², R³, R⁴, R⁵, and R^{F} are as defined for Formula I.

Formula I also includes compounds of Formula III:

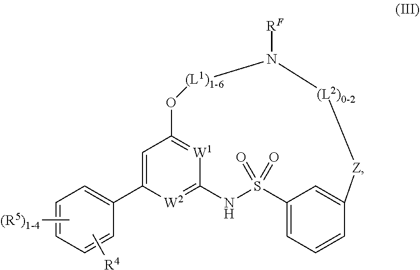

tautomers of those compounds, deuterated derivatives of any of the compounds and tautomers, and pharmaceutically acceptable salts of any of the foregoing, wherein W¹, W², Z, L¹, L², R⁴, R⁵, and R^{F} are as defined for Formula I.

Formula I also includes compounds of Formula IV:

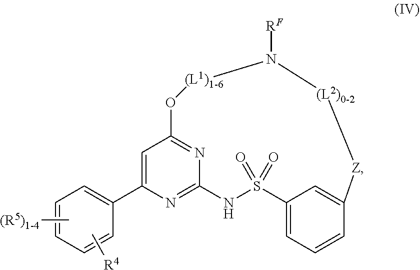

tautomers of those compounds, deuterated derivatives of any of the compounds and tautomers, and pharmaceutically acceptable salts of any of the foregoing, wherein Z, L¹, L², R⁴, R⁵, and R^{F} are as defined for Formula I.

Formula I also includes compounds of Formula V:

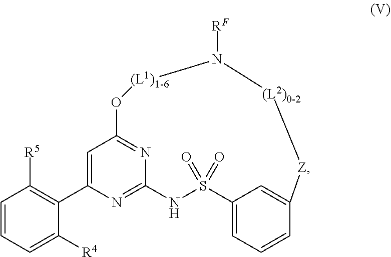

tautomers of those compounds, deuterated derivatives of any of the compounds and tautomers, and pharmaceutically acceptable salts of any of the foregoing, wherein Z, L¹, L², R⁴, R⁵, and R^{F} are as defined for Formula I.

Formula I also includes compounds of Formula Va and Formula Vb:

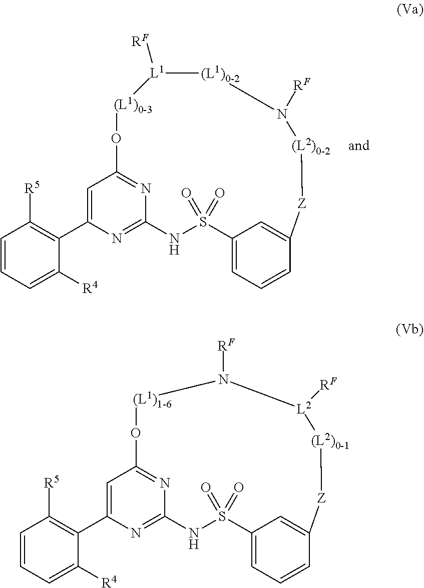

tautomers of those compounds, deuterated derivatives of any of the compounds and tautomers, and pharmaceutically acceptable salts of any of the foregoing, wherein Z, L¹, L², R⁴, R⁵, and R^{F} are as defined for Formula I

Formula I also includes compounds of Formula VI:

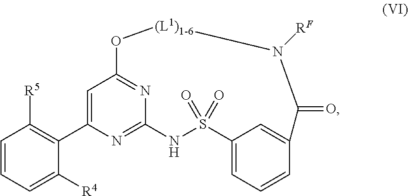

tautomers of those compounds, deuterated derivatives of any of the compounds and tautomers, and pharmaceutically acceptable salts of any of the foregoing, wherein L¹, R⁴, R⁵, and R^{F} defined for Formula I.

Another aspect of the disclosure provides pharmaceutical compositions comprising at least one compound chosen from the novel compounds disclosed herein, tautomers thereof, deuterated derivatives of those compounds and tautomers, and pharmaceutically acceptable salts of any of the foregoing, and at least one pharmaceutically acceptable carrier, which compositions may further include at least one additional active pharmaceutical ingredient. In some embodiments, the at least one additional active pharmaceutical ingredient is at least one other CFTR modulator. In some embodiments, the at least one other CFTR modulator is selected from CFTR potentiators. In some embodiments, the at least one other CFTR modulator is selected from CFTR correctors. In some embodiments, the at least one other CFTR modulator includes a potentiator and corrector. In some embodiments, the at least one other CFTR modulator is selected from tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.

Thus, another aspect of the disclosure provides methods of treating the CFTR-mediated disease cystic fibrosis comprising administering at least one compound chosen from the novel compounds disclosed herein, tautomers thereof, deuterated derivatives of those compounds and tautomers, and pharmaceutically acceptable salts of any of the foregoing, and at least one pharmaceutically acceptable carrier, optionally as part of a pharmaceutical composition comprising at least one additional active pharmaceutical ingredient, to a subject in need thereof. In some embodiments, the at least one additional active pharmaceutical ingredient is at least one other CFTR modulator. In some embodiments, the at least one other CFTR modulator is selected from CFTR potentiators. In some embodiments, the at least one other CFTR modulator is selected from CFTR correctors. In some embodiments, the at least one other CFTR modulator includes a potentiator and corrector. In some embodiments, the at least one other CFTR modulator is selected from tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.

In certain embodiments, the pharmaceutical compositions of the disclosure comprise at least one (i.e., one or more) compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing. In some embodiments, compositions comprising at least one (i.e., one or more) compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing may optionally further comprise (a) at least one (i.e., one or more) compound chosen from (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide (tezacaftor), 3-(6-(1-(2,2-difluorobenzo[d][1,3]dioxol-5-yl)cyclopropane carboxamido)-3-methylpyridin-2-yl)benzoic acid (lumacaftor), and deuterated derivatives and pharmaceutically acceptable salts of tezacaftor and lumacaftor; and/or (b) at least one (i.e., one or more) compound chosen from N-[2,4-bis(1,1-dimethylethyl)-5-hydroxyphenyl]-1,4-dihydro-4-oxoquinoline-3-carboxamide (ivacaftor), N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide (deutivacaftor), (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.

Another aspect of the disclosure provides methods of treating the CFTR-mediated disease, cystic fibrosis, that comprise administering to a patient in need thereof at least one compound chosen from the novel compounds disclosed herein, deuterated derivatives thereof, and pharmaceutically acceptable salts of any of the foregoing, and optionally further administering one or more additional CFTR modulating agents. A further aspect of the disclosure provides the pharmaceutical compositions of the disclosure comprising at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing and, optionally, one or more CFTR modulating agents, for use in therapy or for use in the manufacture of a medicament. In some embodiments, the optional one or more additional CFTR modulating agents are selected from CFTR potentiators. In some embodiments, the one or more additional CFTR modulating agents are selected from CFTR correctors. In some embodiments, the one or more additional CFTR modulating agents are selected from tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.

A further aspect of the disclosure provides intermediates and methods for making the compounds and pharmaceutical compositions disclosed herein.

### Definitions

“Tezacaftor,” as used herein, refers to (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide, which can be depicted with the following structure:

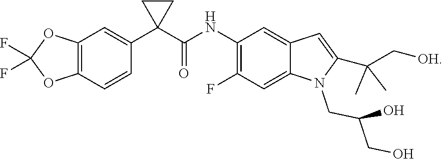 [SMILES: CC(C)(CO)c1cc2cc(NC(=O)C3(c4ccc5c(c4)OC(F)(F)O5)CC3)c(F)cc2n1C[C@@H](O)CO]

Tezacaftor may be in the form of a deuterated derivative or a pharmaceutically acceptable salt, or a pharmaceutically acceptable salt of a deuterated derivative. Tezacaftor and methods of making and using tezacaftor are disclosed in WO 2010/053471, WO 2011/119984, WO 2011/133751, WO 2011/133951, WO 2015/160787, and US 2009/0131492, each of which is incorporated herein by reference.

“Ivacaftor” as used throughout this disclosure refers to N-(2,4-di-tert-butyl-5-hydroxyphenyl)-1,4-dihydro-4-oxoquinoline-3-carboxamide, which is depicted by the structure:

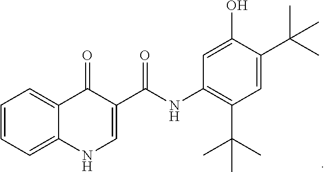 [SMILES: CC(C)(C)c1cc(C(C)(C)C)c(NC(=O)c2c[nH]c3ccccc3c2=O)cc1O]

Ivacaftor may also be in the form of a deuterated derivative, a pharmaceutically acceptable salt, or a pharmaceutically acceptable salt of a deuterated derivative. Ivacaftor and methods of making and using ivacaftor are disclosed in WO 2006/002421, WO 2007/079139, WO 2010/108162, and WO 2010/019239, each of which is incorporated herein by reference.

In some embodiments, a specific deuterated derivative of ivacaftor (deutivacaftor) is employed in the compositions and methods disclosed herein. A chemical name for deutivacaftor is N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide, as depicted by the structure:

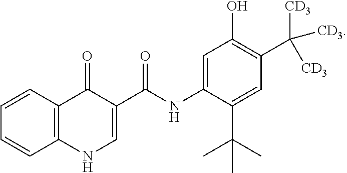 [SMILES: [2H]C([2H])([2H])C(c1cc(C(C)(C)C)c(NC(=O)c2c[nH]c3ccccc3c2=O)cc1O)(C([2H])([2H])[2H])C([2H])([2H])[2H]]

Deutivacaftor may be in the form of a further deuterated derivative, a pharmaceutically acceptable salt, or a pharmaceutically acceptable salt of a further deuterated derivative. Deutivacaftor and methods of making and using deutivacaftor are disclosed in WO 2012/158885, WO 2014/078842, and U.S. Pat. No. 8,865,902, each of which is incorporated herein by reference.

“Lumacaftor” as used herein, refers to 3-(6-(1-(2,2-difluorobenzo[d][1,3]dioxol-5-yl)cyclopropanecarboxamido)-3-methylpyridin-2-yl)benzoic acid, which is depicted by the chemical structure:

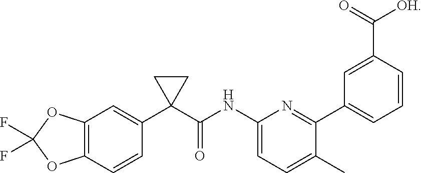 [SMILES: Cc1ccc(NC(=O)C2(c3ccc4c(c3)OC(F)(F)O4)CC2)nc1-c1cccc(C(=O)O)c1]

Lumacaftor may be in the form of a deuterated derivative, a pharmaceutically acceptable salt, or a pharmaceutically acceptable salt of a deuterated derivative. Lumacaftor and methods of making and using lumacaftor are disclosed in WO 2007/056341, WO 2009/073757, and WO 2009/076142, each of which is incorporated herein by reference.

As used herein, the term “alkyl” refers to a saturated or partially saturated, branched or unbranched aliphatic hydrocarbon containing carbon atoms (such as, for example, 1, 2, 3, 4, 5, 6, 7, 8, 9, 10, 11, 12, 13, 14, 15, 16, 17, 18, 19, or 20 carbon atoms), in which one or more bonds between adjacent carbon atoms may be a double (alkenyl) or triple (alkynyl) bond. Alkyl groups may be substituted or unsubstituted.

As used herein, the term “haloalkyl group” refers to an alkyl group substituted with one or more halogen atoms, e.g., fluoroalkyl, which refers to an alkyl group substituted with one or more fluorine atoms.

The term “alkoxy,” as used herein, refers to an alkyl or cycloalkyl covalently bonded to an oxygen atom. Alkoxy groups may be substituted or unsubstituted.

As used herein, the term “haloalkoxyl group” refers to an alkoxy group substituted with one or more halogen atoms.

As used herein, “cycloalkyl” refers to a cyclic, bicyclic, tricyclic, or polycyclic non-aromatic hydrocarbon groups having 3 to 12 carbons (such as, for example 3-10 carbons) and may include one or more unsaturated bonds. “Cycloalkyl” groups encompass monocyclic, bicyclic, tricyclic, bridged, fused, and spiro rings, including mono spiro and dispiro rings. Non-limiting examples of cycloalkyl groups are cyclopropyl, cyclobutyl, cyclopentyl, cyclohexyl, adamantyl, norbornyl, and dispiro[2.0.2.1]heptane. Cycloalkyl groups may be substituted or unsubstituted.

The term “aryl,” as used herein, is a functional group or substituent derived from an aromatic ring and encompasses monocyclic aromatic rings and bicyclic, tricyclic, and fused ring systems, wherein at least one ring in the system is aromatic. Non-limiting examples of aryl groups include phenyl, naphthyl, and 1,2,3,4-tetrahydronaphthalenyl.

The term “heteroaryl ring” as used herein refers to an aromatic ring comprising at least one ring atom that is a heteroatom, such as O, N, or S. Heteroaryl groups encompass monocyclic rings and bicyclic, tricyclic, bridged, fused, and spiro ring systems (including mono spiro and dispiro rings) wherein at least one ring in the system is aromatic. Non-limiting examples of heteroaryl rings include pyridine, quinoline, indole, and indoline.

As used herein, the term “heterocyclyl ring” refers to a non-aromatic hydrocarbon containing 3 to 12 atoms in a ring (such as, for example 3-10 atoms) comprising at least one ring atom that is a heteroatom, such as O, N, or S and may include one or more unsaturated bonds. “Heterocyclyl” rings encompass monocyclic, bicyclic, tricyclic, polycyclic, bridged, fused, and spiro rings, including mono spiro and dispiro rings.

“Substituted,” whether preceded by the term “optionally” or not, indicates that at least one hydrogen of the “substituted” group is replaced by a substituent. Unless otherwise indicated, an “optionally substituted” group may have a suitable substituent at each substitutable position of the group, and when more than one position in any given structure may be substituted with more than one substituent chosen from a specified group, the substituent may be either the same or different at each position.

Examples of protecting groups for nitrogen include, for example, t-butyl carbamate (Boc), benzyl (Bn), para-methoxybenzyl (PMB), tetrahydropyranyl (THP), 9-fluorenylmethyl carbamate (Fmoc), benzyl carbamate (Cbz), methyl carbamate, ethyl carbamate, 2,2,2-trichloroethyl carbamate (Troc), 2-trimethylsilylethyl carbamate (Teoc), allyl carbamate (Aloc or Alloc), formamide, acetamide, benzamide, allylamine, trifluoroacetamide, triphenylmethylamine, benzylideneamine, and p-toluenesulfonamide. A comprehensive list of nitrogen protecting groups can be found in Wuts, P. G. M. “Greene's Protective Groups in Organic Synthesis: Fifth Edition,” 2014, John Wiley and Sons.

As used herein, the terms “selected from” and “chosen from” are used interchangeably.

As used herein, “deuterated derivative(s)” refers to a compound having the same chemical structure as a reference compound, with one or more hydrogen atoms replaced by a deuterium atom. In some embodiments, the one or more hydrogens replaced by deuterium are part of an alkyl group. In some embodiments, the one or more hydrogens replaced by deuterium are part of a methyl group.

As used herein, “CFTR” means cystic fibrosis transmembrane conductance regulator.

The terms “CFTR modulator” and “CFTR modulating agent” are used interchangeably herein to refer to a compound that increases the activity of CFTR. The increase in activity resulting from a CFTR modulator includes but is not limited to compounds that correct, potentiate, stabilize, and/or amplify CFTR.

The terms “corrector” and “CFTR corrector” are used interchangeably herein to refer to a compound that facilitates the processing and trafficking of CFTR to increase the amount of CFTR at the cell surface. The novel compounds disclosed herein are CFTR correctors. Other correctors may be used in combination therapies with the novel compounds disclosed herein to treat CFTR mediated diseases, such as cystic fibrosis. Such other correctors include, e.g., tezacaftor, lumacaftor, and their deuterated derivatives and pharmaceutically acceptable salts.

The terms “potentiator” and “CFTR potentiator” are used interchangeably herein to refer to a compound that increases the channel activity of CFTR protein located at the cell surface, resulting in enhanced ion transport. Ivacaftor and deutivacaftor disclosed herein are CFTR potentiators. Potentiators may be used in combination with the novel compounds of the disclosure to treat CFTR mediated diseases such as cystic fibrosis. Such potentiators include, e.g., ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and their deuterated derivatives and pharmaceutically acceptable salts.

It will be appreciated that when a description of a combination of a compound selected from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, and other specified CFTR modulating agents is provided herein, typically, but not necessarily, the combination or treatment regime will include at least one potentiator, such as, e.g., a potentiator selected from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts thereof. It will also be appreciated that typically, but not necessarily, a single potentiator is used in a combination pharmaceutical composition or therapy. In some embodiments, a combination of at least one compound selected from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, and other specified CFTR modulating agents, will include both a CFTR potentiator, such as, e.g., ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and their deuterated derivatives and pharmaceutically acceptable salts, and another CFTR corrector, such as, e.g., a corrector compound selected from tezacaftor, lumacaftor, and deuterated derivatives and pharmaceutically acceptable salts thereof.

The term “at least one compound selected from,” as used herein, refers to the selection of one or more of the compounds from a specified group.

A reference to “Compounds 1-426 in this disclosure is intended to represent a reference to each of Compounds 1 through 426 individually or a reference to groups of compounds, such as, e.g., Compounds 1-371, Compounds 372-385, and Compounds 386-426.

As used herein, the term “active pharmaceutical ingredient” or “therapeutic agent” (“API”) refers to a biologically active compound.

The terms “patient” and “subject” are used interchangeably and refer to an animal, including a human.

The terms “effective dose” and “effective amount” are used interchangeably herein and refer to that amount of a compound that produces the desired effect for which it is administered (e.g., improvement in CF or a symptom of CF, or lessening the severity of CF or a symptom of CF). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, the terms “treatment,” “treating,” and the like generally mean the improvement in one or more symptoms of CF or lessening the severity of CF or one or more symptoms of CF in a subject. “Treatment,” as used herein, includes, but is not limited to, the following: increased growth of the subject, increased weight gain, reduction of mucus in the lungs, improved pancreatic and/or liver function, reduction of chest infections, and/or reductions in coughing or shortness of breath. Improvements in or lessening the severity of any of these symptoms can be readily assessed according to standard methods and techniques known in the art. It should be understood that references herein to methods of treatment (e.g., methods of treating a CFTR mediated disease or a method of treating cystic fibrosis) using one or more compounds of the disclosure optionally in combination with one or more additional CFTR modulating agents (e.g., a compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, optionally in combination with one or more additional CFTR modulating agents) should also be interpreted as references to:
one or more compounds (e.g., a compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, optionally in combination with one or more additional CFTR modulating agents) for use in methods of treating, e.g., cystic fibrosis optionally in combination with one or more additional CFTR modulating agents; and/or
the use of one or more compounds (e.g., a compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, optionally in combination with one or more additional CFTR modulating agents) in the manufacture of a medicament for treating, e.g., cystic fibrosis.

It should be also understood that references herein to methods of treatment (e.g., methods of treating a CFTR mediated disease or a method of treating cystic fibrosis) using a pharmaceutical composition of the disclosure (e.g., a pharmaceutical composition comprising at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing and optionally further comprising one or more additional CFTR modulating agents) should also be interpreted as references to:
a pharmaceutical composition (e.g., a pharmaceutical composition comprising at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing and optionally further comprising one or more additional CFTR modulating agents) for use in methods of treating, e.g., cystic fibrosis; and/or
the use of a pharmaceutical composition (e.g., a pharmaceutical composition comprising at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing and optionally further comprising one or more additional CFTR modulating agents) in the manufacture of a medicament for treating, e.g., cystic fibrosis.

As used herein, the term “in combination with,” when referring to two or more compounds, agents, or additional active pharmaceutical ingredients, means the administration of two or more compounds, agents, or active pharmaceutical ingredients to the patient prior to, concurrent with, or subsequent to each other.

The terms “about” and “approximately” may refer to an acceptable error for a particular value as determined by one of skill in the art, which depends in part on how the values are measured or determined. In some embodiments, the terms “about” and “approximately” mean within 20%, 15%, 10%, 5%, 4%, 3%, 2%, 1%, or 0.5% of a given value or range.

As used herein, the term “solvent” refers to any liquid in which the product is at least partially soluble (solubility of product>1 g/L).

As used herein, the term “room temperature” or “ambient temperature” means 15° C. to 30° C.

It will be appreciated that certain compounds of this disclosure may exist as separate stereoisomers or enantiomers and/or mixtures of those stereoisomers or enantiomers.

Certain compounds disclosed herein may exist as tautomers and both tautomeric forms are intended, even though only a single tautomeric structure is depicted. For example, a description of Compound X is understood to include its tautomer Compound Y and vice versa, as well as mixtures thereof:

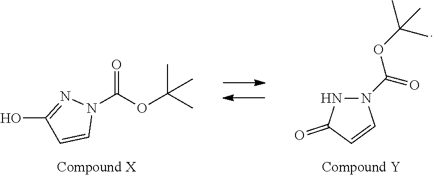

As used herein, “minimal function (MF) mutations” refer to CFTR gene mutations associated with minimal CFTR function (little-to-no functioning CFTR protein) and include, for example, mutations associated with severe defects in ability of the CFTR channel to open and close, known as defective channel gating or “gating mutations”; mutations associated with severe defects in the cellular processing of CFTR and its delivery to the cell surface; mutations associated with no (or minimal) CFTR synthesis; and mutations associated with severe defects in channel conductance.

As used herein, the term “pharmaceutically acceptable salt” refers to a salt form of a compound of this disclosure, wherein the salt is nontoxic. Pharmaceutically acceptable salts of the compounds of this disclosure include those derived from suitable inorganic and organic acids and bases. A “free base” form of a compound, for example, does not contain an ionically bonded salt.

The phrase “and deuterated derivatives and pharmaceutically acceptable salts thereof” is used interchangeably with “and deuterated derivatives and pharmaceutically acceptable salts of any of the forgoing” in reference to one or more compounds or formulae of the disclosure. These phrases are intended to encompass pharmaceutically acceptable salts of any one of the referenced compounds, deuterated derivatives of any one of the referenced compounds, and pharmaceutically acceptable salts of those deuterated derivatives.

One of ordinary skill in the art would recognize that, when an amount of “a compound or a pharmaceutically acceptable salt thereof” is disclosed, the amount of the pharmaceutically acceptable salt form of the compound is the amount equivalent to the concentration of the free base of the compound. It is noted that the disclosed amounts of the compounds or their pharmaceutically acceptable salts thereof herein are based upon their free base form.

Suitable pharmaceutically acceptable salts are, for example, those disclosed in S. M. Berge, et al. J. Pharmaceutical Sciences, 1977, 66, 1-19. For example, Table 1 of that article provides the following pharmaceutically acceptable salts:

TABLE 1
Acetate
Benzenesulfonate
Benzoate
Bicarbonate
Bitartrate
Bromide
Calcium edetate
Camsylate
Carbonate
Chloride
Citrate
Dihydrochloride
Edetate
Edisylate
Estolate
Esylate
Fumarate
Gluceptate
Gluconate
Glutamate
Glycollylarsanilate
Hexylresorcinate
Hydrabamine
Hydrobromide
Hydrochloride
Hydroxynaphthoate
Iodide
Isethionate
Lactate
Lactobionate
Malate
Maleate
Mandelate
Mesylate
Methylbromide
Methylnitrate
Methylsulfate
Mucate
Napsylate
Nitrate
Pamoate (Embonate)
Pantothenate
Phosphate/diphosphate
Polygalacturonate
Salicylate
Stearate
Subacetate
Succinate
Sulfate
Tannate
Tartrate
Teociate
Triethiodide
Benzathine
Chloroprocaine
Choline
Diethanolamine
Ethylenediamine
Meglumine
Procaine
Aluminum
Calcium
Lithium
Magnesium
Potassium
Sodium
Zinc

Non-limiting examples of pharmaceutically acceptable acid addition salts include: salts formed with inorganic acids, such as hydrochloric acid, hydrobromic acid, phosphoric acid, sulfuric acid, or perchloric acid; salts formed with organic acids, such as acetic acid, oxalic acid, maleic acid, tartaric acid, citric acid, succinic acid or malonic acid; and salts formed by using other methods used in the art, such as ion exchange. Non-limiting examples of pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Pharmaceutically acceptable salts derived from appropriate bases include alkali metal, alkaline earth metal, ammonium, and N⁺(C₁₋₄alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

Also disclosed herein are Compounds 1-426, tautomers thereof, deuterated derivatives of those compounds and tautomers, and pharmaceutically acceptable salts of any of the foregoing.

### Methods of Treatment

Any of the novel compounds disclosed herein, such as, for example, compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, can act as a CFTR modulator, i.e., modulating CFTR activity in the body. Individuals suffering from a mutation in the gene encoding CFTR may benefit from receiving a CFTR modulator. A CFTR mutation may affect the CFTR quantity, i.e., the number of CFTR channels at the cell surface, or it may impact CFTR function, i.e., the functional ability of each channel to open and transport ions. Mutations affecting CFTR quantity include mutations that cause defective synthesis (Class I defect), mutations that cause defective processing and trafficking (Class II defect), mutations that cause reduced synthesis of CFTR (Class V defect), and mutations that reduce the surface stability of CFTR (Class VI defect). Mutations that affect CFTR function include mutations that cause defective gating (Class III defect) and mutations that cause defective conductance (Class IV defect). Some CFTR mutations exhibit characteristics of multiple classes. Certain mutations in the CFTR gene result in cystic fibrosis.

Thus, in some embodiments, the disclosure provides methods of treating, lessening the severity of, or symptomatically treating cystic fibrosis in a patient comprising administering to the patient an effective amount of any of the novel compounds disclosed herein, such as, for example, compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, alone or in combination with another active ingredient, such as one or more CFTR modulating agents. In some embodiments, the one (or more) CFTR modulating agent is a corrector. In some embodiments, the one (or more) CFTR modulating agent is a potentiator. In some embodiments, the CFTR modulating agents include both a corrector and a potentiator. In some embodiments, the one or more CFTR modulating agents are selected from potentiators: ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing; and correctors: lumacaftor, tezacaftor, and deuterated derivatives and pharmaceutically acceptable salts thereof.

In some embodiments, the patient to be treated has an F508del/minimal function (MF) genotype, F508del/F508del genotype (homozygous for the F508del mutation), F508del/gating genotype, or F508del/residual function (RF) genotype. In some embodiments, the patient is heterozygous and has one F508del mutation. In some embodiments, the patient is homozygous for the N1303K mutation.

In some embodiments, 5 mg to 500 mg of a compound disclosed herein, a tautomer thereof, deuterated derivatives of the compound and tautomer, or a pharmaceutically acceptable salt of any of the foregoing are administered daily.

In some embodiments, the patient to be treated has at least one F508del mutation in the CFTR gene. In some embodiments, the patient has a CFTR gene mutation that is responsive to a compound, tautomer, deuterated derivative, or pharmaceutically acceptable salt of the disclosure based on in vitro data. In some embodiments, the patient is heterozygous and has an F508del mutation on one allele and a mutation on the other allele selected from Table 2:

TABLE 2
CFTR Mutations

| MF Category | Mutation | | | | |
|---|---|---|---|---|---|
| Nonsense mutations | Q2X | L218X | Q525X | R792X | E1104X |
| | S4X | Q220X | G542X | E822X | W1145X |
| | W19X | Y275X | G550X | W882X | R1158X |
| | G27X | C276X | Q552X | W846X | R1162X |
| | Q39X | Q290X | R553X | Y849X | S1196X |
| | W57X | G330X | E585X | R851X | W1204X |
| | E60X | W401X | G673X | Q890X | L1254X |
| | R75X | Q414X | Q685X | S912X | S1255X |
| | L88X | S434X | R709X | Y913X | W1282X |
| | E92X | S466X | K710X | Q1042X | Q1313X |
| | Q98X | S489X | Q715X | W1089X | Q1330X |
| | Y122X | Q493X | L732X | Y1092X | E1371X |
| | E193X | W496X | R764X | W1098X | Q1382X |
| | W216X | C524X | R785X | R1102X | Q1411X |
| Canonical splice | 185 + 1G→T | 711 + 5G→A | 1717 − 8G→A | 2622 + 1G→A | 3121 − 1G→A |
| mutations | 296 + 1G→A | 712 − 1G→T | 1717 − 1G→A | 2790 − 1G→C | 3500 − 2A→G |
| | 296 + 1G→T | 1248 + 1G→A | 1811 + 1G→C | 3040G→C | 3600 + 2insT |
| | 405 + 1G→A | 1249 − 1G→A | 1811 + 1.6kbA→G | (G970R) | 3850 − 1G→A |
| | 405 + 3A→C | 1341 + 1G→A | 1811 + 1643G→T | 3120G→A | 4005 + 1G→A |
| | 406 − 1G→A | 1525 − 2A→G | 1812 − 1G→A | 3120 + 1G→A | 4374 + 1G→T |
| | 621 + 1G→T | 1525 − 1G→A | 1898 + 1G→A | 3121 − 2A→G | |
| | 711 + 1G→T | | 1898 + 1G→C | | |
| Small (≤3 nucleotide) | 182delT | 1078delT | 1677delTA | 2711delT | 3737delA |
| insertion/deletion | 306insA | 1119delA | 1782delA | 2732insA | 3791delC |
| | 306delTAGA | 1138insG | 1824delA | 2869insG | 3821delT |
| (ins/del) frameshift | 365-366insT | 1154insTC | 1833delT | 2896insAG | 3876delA |
| mutations | 394delTT | 1161delC | 2043delG | 2942insT | 3878delG |
| | 442delA | 1213delT | 2143delT | 2957delT | 3905insT |
| | 444delA | 1259insA | 2183AA→Gª | 3007delG | 4016insT |
| | 457TAT→G | 1288insTA | 2184delA | 3028delA | 4021dupT |
| | 541delC | 1343delG | 2184insA | 3171delC | 4022insT |
| | 574delA | 1471delA | 2307insA | 3171insC | 4040delA |
| | 663delT | 1497delGG | 2347delG | 3271delGG | 4279insA |
| | 849delG | 1548delG | 2585delT | 3349insT | 4326delTC |
| | 935delA | 1609del CA | 2594delGT | 3659delC | |

| | | | |
|---|---|---|---|
| Non-small (>3 | CFTRdele1 | CFTRdele16-17b | 1461ins4 |
| nucleotide) | CFTRdele2 | CFTRdele17a, 17b | 1924del7 |
| insertion/deletion | CFTRdele2, 3 | CFTRdele17a-18 | 2055de19→A |
| (ins/del) frameshift | CFTRdele2-4 | CFTRdele19 | 2105-2117del13insAGAAA |
| mutations | CFTRdele3-10, 14b-16 | CFTRdele 19-21 | 2372del8 |
| | CFTRdele4-7 | CFTRdele21 | 2721del11 |
| | CFTRdele4-11 | CFTRdele22-24 | 2991del32 |
| | CFTR50kbdel | CFTRdele22, 23 | 3667ins4 |
| | CFTRdup6b-10 | 124del23bp | 4010del4 |
| | CFTRdelell | 602del14 | 4209TGTT→AA |
| | CFTRdele13, 14a | 852del22 | |
| | CFTRdele14b-17b | 991de15 | |

| | | | | |
|---|---|---|---|---|
| Missense mutations that | A46D | V520F | Y569D | N1303K |
| Are not responsive in | G85E | A559T | L1065P | |
| vitro to TEZ, IVA, or | R347P | R560T | R1066C | |
| TEZ/IVA | | | | |
| and | L467P | R560S | L1077P | |
| %PI >50% and | I507del | A561E | M1101K | |
| SwCl⁻ >86 mmol/L | | | | |

^{a}Also known as 2183delAA→G.
CFTR: cystic fibrosis transmembrane conductance regulator;
IVA: ivacaftor.
SwCl: sweat chloride.
TEZ: tezacaftor.
Source: CFTR2.org +8 Internet+9 . Baltimore (MD): Clinical and functional translation of CFTR. The Clinical and Functional Translation of CFTR (CFTR2), US Cystic Fibrosis Foundation, Johns Hopkins University, the Hospital for Sick Children. Available at: http://www.cftr2.org/. Accessed 15 May 2018.
Notes:
% PI: percentage of F508del-CFTR heterozygous patients in the CFTR2 patient registry who are pancreatic insufficient; SwCl: mean sweat chloride of F508del-CFTR heterozygous patients in the CFTR2 patient registry.

In some embodiments, the disclosure also is directed to methods of treatment using isotope-labelled compounds of the afore-mentioned compounds, or pharmaceutically acceptable salts thereof, wherein the formula and variables of such compounds and salts are each and independently as described above or any other embodiments described above, provided that one or more atoms therein have been replaced by an atom or atoms having an atomic mass or mass number which differs from the atomic mass or mass number of the atom which usually occurs naturally (isotope labelled). Examples of isotopes which are commercially available and suitable for the disclosure include isotopes of hydrogen, carbon, nitrogen, oxygen, phosphorus, fluorine and chlorine, for example, ²H, ³H, ¹³C, ¹⁴C, ¹⁵N, ¹⁸O, ¹⁷O, ³¹P, ³²p, ³⁵S, ¹⁸F, and ³⁶Cl, respectively.

The isotope-labelled compounds and salts can be used in a number of beneficial ways. They can be suitable for medicaments and/or various types of assays, such as substrate tissue distribution assays. For example, tritium (³H)- and/or carbon-14 (¹⁴C)-labelled compounds are particularly useful for various types of assays, such as substrate tissue distribution assays, due to relatively simple preparation and excellent detectability. For example, deuterium (²H)-labelled ones are therapeutically useful with potential therapeutic advantages over the non-²H-labelled compounds. In general, deuterium (²H)-labelled compounds and salts can have higher metabolic stability as compared to those that are not isotope-labelled owing to the kinetic isotope effect described below. Higher metabolic stability translates directly into an increased in vivo half-life or lower dosages, which could be desired. The isotope-labelled compounds and salts can usually be prepared by carrying out the procedures disclosed in the synthesis schemes and the related description, in the example part, and in the preparation part in the present text, replacing a non-isotope-labelled reactant by a readily available isotope-labelled reactant.

In some embodiments, the isotope-labelled compounds and salts are deuterium (²H)-labelled ones. In some specific embodiments, the isotope-labelled compounds and salts are deuterium (²H)-labelled, wherein one or more hydrogen atoms therein have been replaced by deuterium. In chemical structures, deuterium is represented as “D.”

The concentration of the isotope(s) (e.g., deuterium) incorporated into the isotope-labelled compounds and salt of the disclosure may be defined by the isotopic enrichment factor. The term “isotopic enrichment factor” as used herein means the ratio between the isotopic abundance and the natural abundance of a specified isotope. In some embodiments, if a substituent in a compound of the disclosure is denoted as deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### Combination Therapies

One aspect disclosed herein provides methods of treating cystic fibrosis and other CFTR mediated diseases using any of the novel compounds disclosed herein, such as for example, compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, in combination with at least one additional active pharmaceutical ingredient.

In some embodiments, at least one additional active pharmaceutical ingredient is selected from mucolytic agents, bronchodilators, antibiotics, anti-infective agents, and anti-inflammatory agents.

In some embodiments, the additional therapeutic agent is an antibiotic. Exemplary antibiotics useful herein include tobramycin, including tobramycin inhaled powder (TIP), azithromycin, aztreonam, including the aerosolized form of aztreonam, amikacin, including liposomal formulations thereof, ciprofloxacin, including formulations thereof suitable for administration by inhalation, levoflaxacin, including aerosolized formulations thereof, and combinations of two antibiotics, e.g., fosfomycin and tobramycin.

In some embodiments, the additional agent is a mucolyte. Exemplary mucolytes useful herein includes Pulmozyme®.

In some embodiments, the additional agent is a bronchodilator. Exemplary bronchodilators include albuterol, metaprotenerol sulfate, pirbuterol acetate, salmeterol, or tetrabuline sulfate.

In some embodiments, the additional agent is an anti-inflammatory agent, i.e., an agent that can reduce the inflammation in the lungs. Exemplary such agents useful herein include ibuprofen, docosahexanoic acid (DHA), sildenafil, inhaled glutathione, pioglitazone, hydroxychloroquine, or simvastatin.

In some embodiments, the additional agent is a nutritional agent. Exemplary nutritional agents include pancrelipase (pancreatic enzyme replacement), including Pancrease®, Pancreacarb®, Ultrase®, or Creon®, Liprotomase® (formerly Trizytek®), Aquadeks®, or glutathione inhalation. In one embodiment, the additional nutritional agent is pancrelipase.

In some embodiments, at least one additional active pharmaceutical ingredient is selected from CFTR modulating agents. In some embodiments, the additional active pharmaceutical ingredient is selected from CFTR potentiators. In some embodiments, the potentiator is selected from ivacaftor, deutivacaftor, and (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing. In some embodiments, the additional active pharmaceutical ingredient is chosen from CFTR correctors. In some embodiments, the correctors are selected from lumacaftor, tezacaftor, deuterated derivatives of lumacaftor and tezacaftor, and pharmaceutically acceptable salts of any of the foregoing. In some embodiments, the additional active pharmaceutical ingredient includes both a CFTR potentiator and a CFTR corrector.

In some embodiments, the at least one additional active pharmaceutical ingredient is chosen from (a) tezacaftor, lumacaftor, and deuterated derivatives and pharmaceutically acceptable salts thereof; and (b) ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing. Thus, in some embodiments, the combination therapies provided herein comprise (a) a compound selected from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing; (b) at least one compound selected from tezacaftor, lumacaftor, and deuterated derivatives and pharmaceutically acceptable salts thereof; or (c) at least one compound selected from ivacaftor, deutivacaftor, deuterated derivatives and pharmaceutically acceptable salts of any of the foregoing. In some embodiments, the combination therapies provided herein comprise (a) at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing; (b) at least one compound selected from tezacaftor, lumacaftor, and deuterated derivatives and pharmaceutically acceptable salts thereof, and (c) at least one compound selected from ivacaftor, deutivacaftor, and deuterated derivatives and pharmaceutically acceptable salts of any of the foregoing. In some embodiments, the combination therapies provided herein comprise (a) at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing; (b) at least one compound selected from tezacaftor, lumacaftor, and deuterated derivatives and pharmaceutically acceptable salts thereof; and/or (c) at least one compound selected from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts of any of the foregoing.

In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, is administered in combination with at least one compound chosen from tezacaftor and deuterated derivatives and pharmaceutically acceptable salts thereof. In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, is administered in combination with at least one compound chosen from lumacaftor and deuterated derivatives and pharmaceutically acceptable salts thereof. In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, is administered in combination with at least one compound chosen from ivacaftor and deuterated derivatives and pharmaceutically acceptable salts thereof. In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, is administered in combination with at least one compound chosen from deutivacaftor and deuterated derivatives and pharmaceutically acceptable salts thereof. In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, is administered in combination with at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof.

In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, is administered in combination with at least one compound chosen from tezacaftor and deuterated derivatives and pharmaceutically acceptable salts thereof and at least one compound chosen from ivacaftor and deuterated derivatives and pharmaceutically acceptable salts thereof. In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, is administered in combination with at least one compound chosen from tezacaftor and deuterated derivatives and pharmaceutically acceptable salts thereof and at least one compound chosen from deutivacaftor and deuterated derivatives and pharmaceutically acceptable salts thereof. In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, is administered in combination with at least one compound chosen from tezacaftor and deuterated derivatives and pharmaceutically acceptable salts thereof and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof.

In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, is administered in combination with at least one compound chosen from lumacaftor and deuterated derivatives and pharmaceutically acceptable salts thereof and at least one compound chosen from ivacaftor and deuterated derivatives and pharmaceutically acceptable salts thereof. In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, is administered in combination with at least one compound chosen from lumacaftor and deuterated derivatives and pharmaceutically acceptable salts thereof and at least one compound chosen from deutivacaftor and deuterated derivatives and pharmaceutically acceptable salts thereof. In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, is administered in combination with at least one compound chosen from lumacaftor and deuterated derivatives and pharmaceutically acceptable salts thereof and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof.

Each of the compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, independently can be administered once daily, twice daily, or three times daily. In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, is administered once daily. In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, is administered twice daily.

In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, and at least one compound chosen from tezacaftor and deuterated derivatives and pharmaceutically acceptable salts thereof are administered once daily. In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, and at least one compound chosen from tezacaftor and deuterated derivatives and pharmaceutically acceptable salts thereof are administered twice daily.

In some embodiments, at least one compound chosen from compounds of Formula I, compounds of any one of Formulae Ia, IIa, IIb, III, IV, V, Va, Vb, and VI, Compounds 1-371, Compounds 372-385, Compounds 386-426, tautomers thereof, deuterated derivatives of those compounds and tautomers, and pharmaceutically acceptable salts of any of the foregoing, and at least one compound chosen from ivacaftor, deutivacaftor, and deuterated derivatives and pharmaceutically acceptable salts thereof are administered once daily. In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, and at least one compound chosen from ivacaftor, deutivacaftor, and deuterated derivatives and pharmaceutically acceptable salts thereof are administered twice daily.

In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof are administered once daily. In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof are administered twice daily.

In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, at least one compound chosen from tezacaftor and deuterated derivatives and pharmaceutically acceptable salts thereof, and at least one compound chosen from ivacaftor, deutivacaftor, and deuterated derivatives and pharmaceutically acceptable salts thereof are administered once daily. In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, at least one compound chosen from tezacaftor and deuterated derivatives and pharmaceutically acceptable salts thereof, and at least one compound chosen from ivacaftor, deutivacaftor, and deuterated derivatives and pharmaceutically acceptable salts thereof are administered twice daily.

In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, at least one compound chosen from tezacaftor and deuterated derivatives and pharmaceutically acceptable salts thereof, and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof are administered once daily. In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, at least one compound chosen from tezacaftor and deuterated derivatives and pharmaceutically acceptable salts thereof, and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof are administered twice daily.

In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, at least one compound chosen from ivacaftor, deutivacaftor, and deuterated derivatives and pharmaceutically acceptable salts thereof, and at least one compound chosen from lumacaftor and pharmaceutically acceptable salts thereof, are administered once daily. In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, at least one compound chosen from ivacaftor, deutivacaftor, and deuterated derivatives and pharmaceutically acceptable salts thereof, and at least one compound chosen from lumacaftor and pharmaceutically acceptable salts thereof, are administered twice daily.

In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof, and at least one compound chosen from lumacaftor and pharmaceutically acceptable salts thereof, are administered once daily. In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof, and at least one compound chosen from lumacaftor and pharmaceutically acceptable salts thereof, are administered twice daily.

In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, and at least one compound chosen from tezacaftor and deuterated derivatives and pharmaceutically acceptable salts thereof, are administered once daily and at least one compound chosen from ivacaftor and deuterated derivatives and pharmaceutically acceptable salts thereof, are administered twice daily. In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, and at least one compound chosen from lumacaftor and deuterated derivatives and pharmaceutically acceptable salts thereof, are administered once daily and at least one compound chosen from ivacaftor and deuterated derivatives and pharmaceutically acceptable salts thereof, are administered twice daily.

Compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, and at least one compound selected from tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing can be administered in a single pharmaceutical composition or separate pharmaceutical compositions. Such pharmaceutical compositions can be administered once daily or multiple times daily, such as twice daily or three times daily. As used herein, the phrase that a given amount of API (e.g., tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, or a deuterated derivative or a pharmaceutically acceptable salt of any of the foregoing) is administered once or twice daily or per day means that said given amount is administered per dosing once or twice daily.

In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, is administered in a first pharmaceutical composition; at least one compound chosen from tezacaftor and deuterated derivatives and pharmaceutically acceptable salts thereof is administered in a second pharmaceutical composition; and at least one compound chosen from ivacaftor and deuterated derivatives and pharmaceutically acceptable salts thereof is administered in a third pharmaceutical composition.

In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, is administered in a first pharmaceutical composition; at least one compound chosen from tezacaftor and deuterated derivatives and pharmaceutically acceptable salts thereof is administered in a second pharmaceutical composition; at least one compound chosen from deutivacaftor and deuterated derivatives and pharmaceutically acceptable salts thereof is administered in a third pharmaceutical composition.

In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, is administered in a first pharmaceutical composition; at least one compound chosen from tezacaftor and deuterated derivatives and pharmaceutically acceptable salts thereof is administered in a second pharmaceutical composition; at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof is administered in a third pharmaceutical composition.

In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, is administered in a first pharmaceutical composition; at least one compound chosen from ivacaftor, deutivacaftor, and deuterated derivatives and pharmaceutically acceptable salts thereof is administered in a second pharmaceutical composition; at least one compound chosen from lumacaftor and deuterated derivatives and pharmaceutically acceptable salts thereof is administered in a third pharmaceutical composition.

In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, is administered in a first pharmaceutical composition; at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof is administered in a second pharmaceutical composition; at least one compound chosen from lumacaftor and deuterated derivatives and pharmaceutically acceptable salts thereof is administered in a third pharmaceutical composition.

In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, is administered in a first pharmaceutical composition; and at least one compound chosen from tezacaftor and pharmaceutically acceptable salts thereof and at least one compound chosen from ivacaftor, deutivacaftor, and deuterated derivatives and pharmaceutically acceptable salts thereof are administered in a second pharmaceutical composition. In some embodiments, the second pharmaceutical composition comprises a half of a daily dose of ivacaftor or a pharmaceutically acceptable salt thereof, and the other half of the daily dose of ivacaftor or a pharmaceutically acceptable salt thereof is administered in a third pharmaceutical composition.

In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, is administered in a first pharmaceutical composition; and at least one compound chosen from tezacaftor and pharmaceutically acceptable salts thereof and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof are administered in a second pharmaceutical composition.

In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing; at least one compound chosen from tezacaftor and pharmaceutically acceptable salts thereof and at least one compound chosen from ivacaftor, deutivacaftor, and deuterated derivatives and pharmaceutically acceptable salts thereof are administered in a first pharmaceutical composition. In some embodiments, the first pharmaceutical composition is administered to the patient twice daily. In some embodiments, the first pharmaceutical composition is administered once daily. In some embodiments, the first pharmaceutical composition is administered once daily and a second composition comprising only ivacaftor is administered once daily.

In some embodiments, at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing; at least one compound chosen from tezacaftor and pharmaceutically acceptable salts thereof and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof are administered in a first pharmaceutical composition. In some embodiments, the first pharmaceutical composition is administered to the patient twice daily. In some embodiments, the first pharmaceutical composition is administered once daily. In some embodiments, the first pharmaceutical composition is administered once daily and a second composition comprising only (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol (or a deuterated derivative or pharmaceutically acceptable salt thereof) is administered once daily.

Any suitable pharmaceutical compositions can be used for compounds of Formula I, compounds of any one of Formulae Ia, IIa, IIb, III, IV, V, Va, Vb, and VI, Compounds 1-426, tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and tautomers thereof, deuterated derivatives of those compounds and tautomers, and pharmaceutically acceptable salts of any of the foregoing. Some exemplary pharmaceutical compositions for tezacaftor and its pharmaceutically acceptable salts can be found in WO 2011/119984 and WO 2014/014841, each of which is incorporated herein by reference. Some exemplary pharmaceutical compositions for ivacaftor and its pharmaceutically acceptable salts can be found in WO 2007/134279, WO 2010/019239, WO 2011/019413, WO 2012/027731, and WO 2013/130669, and some exemplary pharmaceutical compositions for deutivacaftor and its pharmaceutically acceptable salts can be found in U.S. Pat. Nos. 8,865,902, 9,181,192, 9,512,079, WO 2017/053455, and WO 2018/080591, all of which are incorporated herein by reference. Some exemplary pharmaceutical compositions for lumacaftor and its pharmaceutically acceptable salts can be found in WO 2010/037066, WO 2011/127421, and WO 2014/071122, all of which are incorporated herein by reference.

### Pharmaceutical Compositions

Another aspect of the disclosure provides a pharmaceutical composition comprising at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, and at least one pharmaceutically acceptable carrier.

In some embodiments, the disclosure provides pharmaceutical compositions comprising at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, in combination with at least one additional active pharmaceutical ingredient. In some embodiments, the at least one additional active pharmaceutical ingredient is a CFTR modulator. In some embodiments, the at least one additional active pharmaceutical ingredient is a CFTR corrector. In some embodiments, the at least one additional active pharmaceutical ingredient is a CFTR potentiator. In some embodiments, the pharmaceutical composition comprises at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, and at least two additional active pharmaceutical ingredients, one of which is a CFTR corrector and one of which is a CFTR potentiator.

In some embodiments, the disclosure provides a pharmaceutical composition comprising (a) at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, (b) at least one compound chosen from tezacaftor and deuterated derivatives and pharmaceutically acceptable salts thereof, and (c) at least one pharmaceutically acceptable carrier. In some embodiments, the disclosure provides a pharmaceutical composition comprising (a) at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, (b) at least one compound chosen from lumacaftor and deuterated derivatives and pharmaceutically acceptable salts thereof, and (c) at least one pharmaceutically acceptable carrier.

In some embodiments, the disclosure provides a pharmaceutical composition comprising (a) at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, (b) at least one compound chosen from ivacaftor, deutivacaftor, and deuterated derivatives and pharmaceutically acceptable salts thereof, and (c) at least one pharmaceutically acceptable carrier.

In some embodiments, the disclosure provides a pharmaceutical composition comprising (a) at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, (b) at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof, and (c) at least one pharmaceutically acceptable carrier.

In some embodiments, the disclosure provides a pharmaceutical composition comprising (a) at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, (b) at least one compound chosen from tezacaftor and deuterated derivatives and pharmaceutically acceptable salts thereof, (c) at least one compound chosen from ivacaftor and deuterated derivatives and pharmaceutically acceptable salts thereof, and (d) at least one pharmaceutically acceptable carrier.

In some embodiments, the disclosure provides a pharmaceutical composition comprising (a) at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, (b) at least one compound chosen from tezacaftor and deuterated derivatives and pharmaceutically acceptable salts thereof, (c) at least one compound chosen from deutivacaftor and deuterated derivatives and pharmaceutically acceptable salts thereof, and (d) at least one pharmaceutically acceptable carrier.

In some embodiments, the disclosure provides a pharmaceutical composition comprising (a) at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, (b) at least one compound chosen from ivacaftor, deutivacaftor, and deuterated derivatives and pharmaceutically acceptable salts thereof, (c) at least one compound chosen from lumacaftor and pharmaceutically acceptable salts thereof, and (d) at least one pharmaceutically acceptable carrier.

In some embodiments, the disclosure provides a pharmaceutical composition comprising (a) at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, (b) at least one compound chosen from tezacaftor and deuterated derivatives and pharmaceutically acceptable salts thereof, (c) at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof, and (d) at least one pharmaceutically acceptable carrier. In some embodiments, the disclosure provides a pharmaceutical composition comprising (a) at least one compound chosen from compounds of Formula I, compounds of any one of Formulae Ia, IIa, IIb, III, IV, V, Va, Vb, and VI, Compounds 1-426, tautomers thereof, deuterated derivatives of those compounds and tautomers, and pharmaceutically acceptable salts of any of the foregoing, (b) at least one compound chosen from lumacaftor and deuterated derivatives and pharmaceutically acceptable salts thereof, (c) at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof, and (d) at least one pharmaceutically acceptable carrier.

Any pharmaceutical composition disclosed herein may comprise at least one pharmaceutically acceptable carrier. In some embodiments, the at least one pharmaceutically acceptable carrier is chosen from pharmaceutically acceptable vehicles and pharmaceutically acceptable adjuvants. In some embodiments, the at least one pharmaceutically acceptable carrier is chosen from pharmaceutically acceptable fillers, disintegrants, surfactants, binders, and lubricants.

The pharmaceutical compositions described herein are useful for treating cystic fibrosis and other CFTR mediated diseases.

As described above, pharmaceutical compositions disclosed herein may optionally further comprise at least one pharmaceutically acceptable carrier. The at least one pharmaceutically acceptable carrier may be chosen from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

### EXEMPLARY EMBODIMENTS

A non-limiting list of embodiments is provided below:

1. A compound of Formula I:

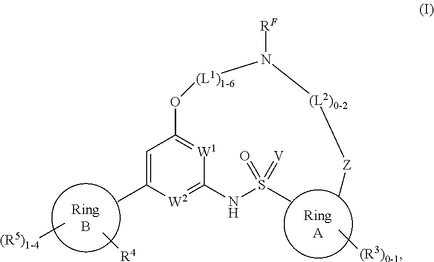

a tautomer thereof, a deuterated derivative of the compound or tautomer, or a pharmaceutically acceptable salt of any of the foregoing, wherein:
   Ring A is selected from:
      C₆-C₁₀ aryl,
      C₃-C₁₀ cycloalkyl,
      3- to 10-membered heterocyclyl, and
      5- to 10-membered heteroaryl;
   Ring B is selected from:
      C₆-C₁₀ aryl,
      C₃-C₁₀ cycloalkyl,
      3- to 10-membered heterocyclyl, and
      5- to 10-membered heteroaryl;
   V is selected from O and NH;
   W¹ is selected from N and CH;
   W² is selected from N and CH; provided that at least one of W¹ and W² is N;
   Z is selected from O, NR^{ZN}, and C(R^{ZC})₂, provided that when L² is absent, Z is C(R^{ZC})₂.
   each L¹ is independently selected from C(R^{L1})₂;
   each L² is independently selected from C(R^{L2})₂;
   each R³ is independently selected from:
      halogen,
      C₁-C₆ alkyl,
      C₁-C₆ alkoxy,
      C₃-C₁₀ cycloalkyl,
      C₆-C₁₀ aryl optionally substituted with 1-3 groups independently selected from C₁-C₆ alkyl, and
      3- to 10-membered heterocyclyl;
   R⁴ is selected from hydrogen and C₁-C₆ alkyl;
   each R⁵ is independently selected from:
      hydrogen,
      halogen,
      hydroxyl,
      N(RN)₂,
      —SO-Me,
      —CH═C(R^{LC})₂, wherein both R^{LC} are taken together to form a C₃-C₁₀ cycloalkyl,
      C₁-C₆ alkyl optionally substituted with 1-3 groups independently selected from:
         hydroxyl,
         C₁-C₆ alkoxy optionally substituted with 1-3 groups independently selected from C₁-C₆ alkoxy and C₆-C₁₀ aryl,
         C₃-C₁₀ cycloalkyl,
         —(O)₀₋₁—(C₆-C₁₀ aryl) optionally substituted with 1-3 groups independently selected from C₁-C₆ alkyl and C₁-C₆ alkoxy,
         3- to 10-membered heterocyclyl, and
         N(RN)₂,
      C₁-C₆ alkoxy optionally substituted with 1-3 groups independently selected from:
         halogen,
         C₆-C₁₀ aryl, and
         C₃-C₁₀ cycloalkyl optionally substituted with 1-3 groups independently selected from C₁-C₆ fluoroalkyl,
      C₁-C₆ fluoroalkyl,
      C₃-C₁₀ cycloalkyl,
      C₆-C₁₀ aryl, and
      3- to 10-membered heterocyclyl;
   R^{ZN} is selected from:
      hydrogen,
      C₁-C₉ alkyl optionally substituted with 1-3 groups independently selected from:
         hydroxyl,
         oxo,
         cyano,
         C₁-C₆ alkoxy optionally substituted with 1-3 groups independently selected from halogen and C₁-C₆ alkoxy,
         N(RN)₂,
         SO₂Me,
         C₃-C₁₀ cycloalkyl optionally substituted with 1-3 groups independently selected from:
         hydroxyl,
         —C₁-C₆ alkyl optionally substituted with 1-3 groups independently selected from hydroxyl, oxo, C₁-C₆ alkoxy, C₆-C₁₀ aryl, and N(R^{N})₂,
         C₁-C₆ fluoroalkyl,
         C₁-C₆ alkoxy, and
         COOH,
         N(R^{N})₂,
         C₆-C₁₀ aryl, and
         3- to 10-membered heterocyclyl optionally substituted with 1-3 groups independently selected from oxo and C₁-C₆ alkyl,
         C₆-C₁₀ aryl optionally substituted with 1-3 groups independently selected from:
         halogen,
         hydroxyl,
         cyano,
         SiMe₃,
         SO₂Me,
         SF₅,
         N(R^{N})₂,
         P(O)Me₂,
         —(O)₀₋₁—(C₃-C₁₀ cycloalkyl) optionally substituted with 1-3 groups independently selected from C₁-C₆ fluoroalkyl,
         C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
         C₁-C₆ alkoxy optionally substituted with 1-3 groups independently selected from hydroxyl, oxo, N(R^{N})₂, and C₆-C₁₀ aryl,
         C₁-C₆ fluoroalkyl,
         3- to 10-membered heterocyclyl optionally substituted with 1-3 groups independently selected from C₁-C₆ alkyl,
         —(O)₀₋₁—(C₆-C₁₀ aryl), and
         —(O)₀₋₁-(5- to 10-heteroaryl) optionally substituted with hydroxyl, oxo, N(R^{N})₂, C₁-C₆ alkyl, C₁-C₆ alkoxy, C₁-C₆ fluoroalkyl, and C₃-C₁₀ cycloalkyl,
         3- to 10-membered heterocyclyl optionally substituted with 1-4 groups independently selected from:
         hydroxyl,
         oxo,
         N(R^{N})₂,
         C₁-C₆ alkyl (optionally substituted with 1-3 groups independently selected from oxo and C₁-C₆ alkoxy),
         C₁-C₆ alkoxy,
         C₁-C₆ fluoroalkyl,
         C₆-C₁₀ aryl optionally substituted with 1-3 groups independently selected from halogen, and
         5- to 10-membered heteroaryl, and
         5- to 10-membered heteroaryl optionally substituted with 1-3 groups independently selected from:
         hydroxyl,
         cyano,
         oxo,
         halogen,
         B(OH)₂,
         N(R^{N})₂,
         C₁-C₆ alkyl optionally substituted with 1-3 groups independently selected from hydroxyl, oxo, C₁-C₆ alkoxy (optionally substituted with 1-3-SiMe₃), and N(R^{N})₂,
         C₁-C₆ alkoxy optionally substituted with 1-3 groups independently selected from hydroxyl, oxo, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from C₁-C₆ alkyl,
         —(O)₀₋₁—(C₆-C₁₀ aryl),
         —(O)₀₋₁-(3- to 10-membered heterocyclyl) optionally substituted with 1-4 groups independently selected from hydroxyl, oxo, halogen, cyano, N(R^{N})₂, C₁-C₆ alkyl (optionally substituted with 1-3 groups independently selected from hydroxyl, oxo, N(R^{N})₂, and C₁-C₆ alkoxy), C₁-C₆ alkoxy, C₁-C₆ fluoroalkyl, 3- to 10-membered heterocyclyl (optionally substituted with 1-3 groups independently selected from C₁-C₆ fluoroalkyl) and
         5- to 10-membered heteroaryl optionally substituted with 1-4 groups independently selected from C₁-C₆ alkyl and C₃-C₁₀ cycloalkyl,
      C₁-C₆ fluoroalkyl,
      C₃-C₁₀ cycloalkyl optionally substituted with 1-3 groups independently selected from:
         hydroxyl,
         oxo,
         halogen,
         cyano,
         N(R^{N})₂,
         C₁-C₆ alkyl optionally substituted with 1-3 groups independently selected from:
         hydroxyl,
         oxo,
         N(R^{N})₂,
         C₁-C₆ alkoxy, and
         C₆-C₁₀ aryl,
         C₁-C₆ alkoxy optionally substituted with 1-3 groups independently selected from halogen, oxo, C₆-C₁₀ aryl, and N(R^{N})₂,
         halogen,
         C₃-C₁₀ cycloalkyl,
         3- to 10-member heterocyclyl optionally substituted with 1-3 groups independently selected from C₁-C₆ alkyl, and
         5- to 10-membered heteroaryl optionally substituted with 1-3 groups independently selected from:
         hydroxyl,
         cyano,
         oxo,
         halogen,
         N(R^{N})₂,
         C₁-C₆ alkyl optionally substituted with 1-3 groups independently selected from hydroxyl, oxo, C₁-C₆ alkoxy, and N(R^{N})₂,
         C₁-C₆ alkoxy optionally substituted with 1-3 groups independently selected from hydroxyl, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from C₁-C₆ alkyl,
         C₆-C₁₀ aryl, and
         3- to 10-membered heterocyclyl optionally substituted with 1-3 groups independently selected from C₁-C₆ alkyl,
      C₆-C₁₀ aryl,
      3- to 10-membered heterocyclyl optionally substituted with 1-3 groups independently selected from:
         oxo,
         C₁-C₆ alkyl optionally substituted with 1-3 groups independently selected from:
         oxo,
         hydroxyl,
         N(R^{N})₂,
         C₁-C₆ alkoxy optionally substituted with 1-3 groups independently selected from halogen and C₆-C₁₀ aryl, and
         —(O)₀₋₁—(C₃-C₁₀ cycloalkyl),
         C₁-C₆ fluoroalkyl,
         C₃-C₁₀ cycloalkyl optionally substituted with 1-3 groups independently selected from halogen, and
         3- to 10-membered heterocyclyl,
      5- to 10-membered heteroaryl optionally substituted with 1-3 groups independently selected from:
         halogen,
         C₁-C₆ alkyl optionally substituted with 1-3 groups independently selected from oxo, C₁-C₆ alkoxy, and N(R^{N})₂, and
         3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl), and
      R^{F}.
   each R^{ZC} is independently selected from:
      hydrogen,
      C₁-C₆ alkyl optionally substituted with 1-3 groups independently selected from C₆-C₁₀ aryl (optionally substituted with 1-3 groups independently selected from C₁-C₆ alkyl),
      C₆-C₁₀ aryl optionally substituted with 1-3 groups independently selected from C₁-C₆ alkyl, and
      R^{F};
   or two R^{ZC} are taken together to form an oxo group;
   each R^{L1} is independently selected from:
      hydrogen,
      N(R^{N})₂, provided that two N(R^{N})₂ are not bonded to the same carbon,
      C₁-C₉ alkyl optionally substituted with 1-3 groups independently selected from:
         halogen,
         hydroxyl,
         oxo,
         N(R^{N})₂,
         C₁-C₆ alkoxy optionally substituted with 1-3 groups independently selected from C₆-C₁₀ aryl,
         C₃-C₁₀ cycloalkyl optionally substituted with 1-3 groups independently selected from halogen and C₁-C₆ fluoroalkyl,
         C₆-C₁₀ aryl optionally substituted with 1-3 groups independently selected from C₁-C₆ alkyl, and
         3- to 10-membered heterocyclyl optionally substituted with 1-3 groups independently selected from C₁-C₆ alkyl (optionally substituted with 1-3 groups independently selected from hydroxyl and oxo),
      C₃-C₁₀ cycloalkyl,
      C₆-C₁₀ aryl optionally substituted with 1-4 groups independently selected from:
         halogen,
         cyano,
         SiMe₃,
         POMe₂,
         C₁-C₇ alkyl optionally substituted with 1-3 groups independently selected from:
         hydroxyl,
         oxo,
         cyano,
         SiMe₃,
         N(R^{N})₂, and
         C₃-C₁₀ cycloalkyl optionally substituted with 1-3 groups independently selected from C₁-C₆ fluoroalkyl,
         C₁-C₆ alkoxy optionally substituted with 1-3 groups independently selected from:
         C₃-C₁₀ cycloalkyl optionally substituted with 1-3 groups independently selected from C₁-C₆ fluoroalkyl, and
         C₁-C₆ alkoxy,
         C₁-C₆ fluoroalkyl,
         C₃-C₁₀ cycloalkyl optionally substituted with 1-3 groups independently selected from C₁-C₆ alkyl and C₁-C₆ fluoroalkyl,
         C₆-C₁₀ aryl,
         3- to 10-membered heterocyclyl optionally substituted with 1-3 groups independently selected from C₁-C₆ alkyl, and
         5- to 10-membered heteroaryl,
      3- to 10-membered heterocyclyl optionally substituted with 1-3 groups independently selected from:
         C₁-C₆ alkyl optionally substituted with 1-3 groups independently selected from:
         oxo, and
         C₁-C₆ alkoxy,
      5- to 10-membered heteroaryl optionally substituted with 1-3 groups independently selected from:
         C₁-C₆ alkyl optionally substituted with 1-3 groups independently selected from:
         C₃-C₁₀ cycloalkyl optionally substituted with 1-3 groups independently selected from C₁-C₆ fluoroalkyl, and
         C₆-C₁₀ aryl optionally substituted with 1-3 groups independently selected from C₁-C₆ alkyl, and
      R^{F};
   or two R^{L1} on the same carbon atom are taken together to form an oxo group;
   each R^{L2} is independently selected from hydrogen and R^{F}; or two R^{L2} on the same carbon atom are taken together to form an oxo group; provided that at least one R^{L1} or R^{L2} is R^{F};
   each R^{N} is independently selected from:
      hydrogen,
      C₁-C₈ alkyl optionally substituted with 1-3 groups independently selected from:
         oxo,
         halogen,
         hydroxyl,
         NH₂,
         NHMe,
         NMe₂,
         C₁-C₆ alkoxy optionally substituted with 1-3 groups independently selected from C₆-C₁₀ aryl,
         —(O)₀₋₁—(C₃-C₁₀ cycloalkyl),
         C₆-C₁₀ aryl optionally substituted with 1-3 groups independently selected from halogen and C₁-C₆ alkyl,
         3- to 14-membered heterocyclyl optionally substituted with 1-4 groups independently selected from oxo and C₁-C₆ alkyl, and
         5- to 14-membered heteroaryl optionally substituted with 1-4 groups independently selected from oxo and C₁-C₆ alkyl,
      C₃-C₁₀ cycloalkyl optionally substituted with 1-3 groups independently selected from:
         hydroxyl,
         NH₂, and
         NHMe, and
         C₁-C₆ alkyl optionally substituted with 1-3 groups independently selected from hydroxyl,
      C₆-C₁₀ aryl, and
      3- to 10-membered heterocyclyl;
   or two R^{N} on the same nitrogen atom are taken together with the nitrogen to which they are bonded to form a 3- to 10-membered heterocyclyl optionally substituted with 1-3 groups selected from:
      hydroxyl,
      oxo,
      cyano,
      C₁-C₆ alkyl optionally substituted with 1-3 groups independently selected from oxo, hydroxyl, C₁-C₆ alkoxy, and N(R^{N2})₂, wherein each R^{N2} is independently selected from hydrogen and C₁-C₆ alkyl,
      C₁-C₆ alkoxy, and
      C₁-C₆ fluoroalkyl;
   or one R⁴ and one R^{L1} are taken together to form a C₆-C₈ alkylene;
   two R^{F} taken together with the atoms to which they are bonded form a group selected from:
      C₃-C₁₀ cycloalkyl optionally substituted with 1-3 groups independently selected from C₁-C₆ alkyl,
      C₆-C₁₀ aryl optionally substituted with 1-3 groups independently selected from:
         halogen,
         C₁-C₆ alkyl,
         N(R^{N})₂, and
         3- to 10-membered heterocyclyl optionally substituted with 1-3 groups independently selected from hydroxyl,
      3- to 11-membered heterocyclyl optionally substituted with 1-3 groups independently selected from:
         oxo,
         N(R^{N})₂,
         C₁-C₉ alkyl optionally substituted with 1-4 groups independently selected from:
         oxo,
         halogen,
         hydroxyl,
         N(R^{N})₂,
         -SO₂—(C₁-C₆ alkyl),
         C₁-C₆ alkoxy optionally substituted with 1-3 groups independently selected from halogen, C₆-C₁₀ aryl,
         C₆-C₁₀ aryl optionally substituted with 1-3 groups independently selected from hydroxyl, halogen, cyano, C₁-C₆ alkyl (optionally substituted with 1-3 groups independently selected from oxo and C₁-C₆ alkoxy), C₁-C₆ alkoxy (optionally substituted with 1-3 groups independently selected from C₆-C₁₀ aryl), —(O)₀₋₁—(C₁-C₆ fluoroalkyl), and C₆-C₁₀ aryl (optionally substituted with 1-3 groups independently selected from C₁-C₆ alkoxy),
         —(O)_{0.1}—(C₃-C₁₀ cycloalkyl) optionally substituted with 1-4 groups independently selected from hydroxyl, halogen, N(R^{N})₂, C₁-C₆ alkyl (optionally substituted with 1-3 groups independently selected from oxo, hydroxyl, and C₁-C₆ alkoxy), C₁-C₆ fluoroalkyl, and C₆-C₁₀ aryl,
         3- to 10-membered heterocyclyl optionally substituted with 1-3 groups independently selected from oxo, C₁-C₆ alkyl (optionally substituted with 1-3 groups independently selected from C₆-C₁₀ aryl (optionally substituted with 1-3 groups independently selected from halogens)), C₁-C₆ alkoxy, C₃-C₁₀ cycloalkyl, and R^{N},
         -O-(5- to 12-membered heteroaryl) optionally substituted with 1-3 groups independently selected from C₆-C₁₀ aryl (optionally substituted with 1-3 groups independently selected from halogen) and C₁-C₆ alkyl, and
         5- to 10-membered heteroaryl optionally substituted with 1-3 groups independently selected from hydroxyl, oxo, N(R^{N})₂, C₁-C₆ alkyl (optionally substituted with 1-3 groups independently selected from cyano), C₁-C₆ alkoxy, —(O)₀₋₁-(C₁-C₆ fluoroalkyl), —O—(C₆-C₁₀ aryl), and C₃-C₁₀ cycloalkyl,
         C₃-C₁₂ cycloalkyl optionally substituted with 1-4 groups independently selected from halogen, C₁-C₆ alkyl, and C₁-C₆ fluoroalkyl,
         C₆-C₁₀ aryl,
         3- to 10-membered heterocyclyl, and
         5- to 10-membered heteroaryl optionally substituted with 1-3 groups independently selected from C₁-C₆ alkoxy, C₁-C₆ fluoroalkyl, and N(R^{N})₂, and
      5- to 12-membered heteroaryl optionally substituted with 1-3 groups independently selected from C₁-C₆ alkyl and C₁-C₆ fluoroalkyl.

1A. In some instances of Embodiment 1, when two R^{F} are taken together form a 3- to 11-membered heterocyclyl, wherein the 3- to 11-membered heterocyclyl is optionally substituted with a 5- to 10-membered heteroaryl, and wherein the 5- to 10-membered heteroaryl is optionally substituted with a C₁-C₆ alkoxy, the C₁-C₆ alkoxy may be optionally substituted with C₆-C₁₀ aryl.

2. The compound, salt, or deuterated derivative according to embodiment 1, wherein Ring A is selected from C₆-C₁₀ aryl, 3- to 10-membered heterocyclyl, and 5- to 10-membered heteroaryl.

3. The compound, salt, or deuterated derivative according to embodiment 1 or 2, wherein Ring A is selected from phenyl, pyridinyl, pyrazolyl, 1H-pyrrolyl, indolinyl, and piperidinyl.

4. The compound, salt, or deuterated derivative according to any one of embodiments 1 to 3, wherein Ring A is phenyl.

5. The compound, salt, or deuterated derivative according to any one of embodiments 1 to 4, wherein Ring B is selected from C₆-C₁₀ aryl.

6. The compound, salt, or deuterated derivative according to any one of embodiments 1 to 5, wherein Ring B is phenyl.

7. The compound, salt, or deuterated derivative according to any one of embodiments 1 to 6, wherein V is O.

8. The compound, salt, or deuterated derivative according to any one of embodiments 1 to 6, wherein V is NH.

9. The compound, salt, or deuterated derivative according to any one of embodiments 1 to 8, wherein W¹ is N and W² is N.

10. The compound, salt, or deuterated derivative according to any one of embodiments 1 to 9, wherein Z is selected from NR^{ZN} and C(R^{ZC}).

11. The compound, salt, or deuterated derivative according to any one of embodiments 1 to 10, wherein each R³ is independently selected from C₁-C₆ alkyl.

12. The compound, salt, or deuterated derivative according to any one of embodiments 1 to 11, wherein each R³ is methyl.

13. The compound, salt, or deuterated derivative according to any one of embodiments 1 to 10, wherein R³ is absent.

14. The compound, salt, or deuterated derivative according to any one of embodiments 1 to 13, wherein R⁴ is selected from hydrogen and methyl.

15. The compound, salt, or deuterated derivative according to any one of embodiments 1 to 14, wherein R⁴ is methyl.

16. The compound, salt, or deuterated derivative according to any one of embodiments 1 to 14, wherein R⁴ is hydrogen.

17. The compound, salt, or deuterated derivative according to any one of embodiments 1 to 16, wherein each R⁵ is independently selected from C₁-C₆ alkyl and C₁-C₆ alkoxy.

18. The compound, salt, or deuterated derivative according to any one of embodiments 1 to 17, wherein each R⁵ is independently selected from methyl,

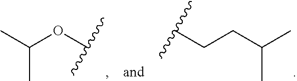

19. The compound, salt, or deuterated derivative according to any one of embodiments 1 to 18, wherein R^{ZN} is selected from hydrogen and R^{F}.

20. The compound, salt, or deuterated derivative according to any one of embodiments 1 to 19, wherein R^{ZN} is hydrogen.

21. The compound, salt, or deuterated derivative according to any one of embodiments 1 to 19, wherein R^{ZN} is R^{F}

22. The compound, salt, or deuterated derivative according to any one of embodiments 1 to 21, wherein R^{ZC} is hydrogen, or two R^{ZC} are taken together to form an oxo group.

23. The compound, salt, or deuterated derivative according to any one of embodiments 1 to 22, wherein each R^{L1} is independently selected from hydrogen, C₁-C₉ alkyl optionally substituted with 1-3 groups independently selected from C₆-C₁₀ aryl, and R^{F}.

24. The compound, salt, or deuterated derivative according to any one of embodiments 1 to 23, wherein each R¹² is independently selected from hydrogen and R^{F}, or two R^{L2} on the same carbon atom are taken together to form an oxo group.

25. The compound, salt, or deuterated derivative according to any one of embodiments 1 to 24, wherein each R^{N} is independently selected from hydrogen and C₁-C₈ alkyl (optionally substituted with 1-3 groups independently selected from oxo, C₁-C₆ alkoxy, C₃-C₁₀ cycloalkyl, and C₆-C₁₀ aryl).

26. The compound, salt, or deuterated derivative according to any one of embodiments 1 to 25, wherein two R^{F} taken together with the atoms to which they are bonded form a group selected from:
C₆-C₁₀ aryl, and
3- to 11-membered heterocyclyl optionally substituted with 1-3 groups independently selected from:
   oxo,
   C₁-C₉ alkyl optionally substituted with 1-4 groups independently selected from:
      oxo,
      halogen,
      hydroxyl,
      N(R^{N})₂,
      C₁-C₆ alkoxy optionally substituted with 1-3 groups independently selected from C₆-C₁₀ aryl,
      C₆-C₁₀ aryl optionally substituted with 1-3 groups independently selected from hydroxyl, cyano, and C₁-C₆ alkyl,
      —(O)₀₋₁—(C₃-C₁₀ cycloalkyl) optionally substituted with 1-4 groups independently selected from N(R^{N})₂, C₁-C₆ alkyl (optionally substituted with 1-3 groups independently selected from oxo, hydroxyl, and C₁-C₆ alkoxy), and C₁-C₆ fluoroalkyl,
      3- to 10-membered heterocyclyl optionally substituted with 1-3 groups independently selected from oxo and C₁-C₆ alkyl, and
      5- to 10-membered heteroaryl optionally substituted with 1-3 groups independently selected from N(R^{N})₂, C₁-C₆ alkyl, and —O—(C₆-C₁₀ aryl),
   C₃-C₁₂ cycloalkyl optionally substituted with 1-4 groups independently selected from halogen and C₁-C₆ alkyl,
   C₆-C₁₀ aryl, and
   3- to 10-membered heterocyclyl.

27. A compound of Formula Ia:

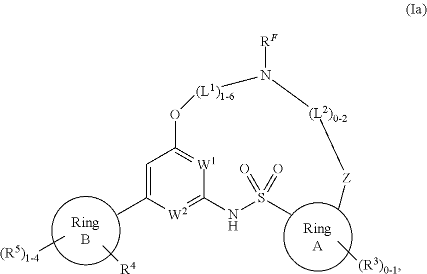

a tautomer thereof, a deuterated derivative of the compound or tautomer, or a pharmaceutically acceptable salt of any of the foregoing, wherein Ring A, Ring B, W¹, W², Z, L¹, L², R³, R⁴, R⁵, and R^{F} are defined as according to embodiment 1.

28. The compound, salt, or deuterated derivative according to embodiment 27, wherein Ring A is selected from C₆-C₁₀ aryl, 3- to 10-membered heterocyclyl, and 5- to 10-membered heteroaryl.

29. The compound, salt, or deuterated derivative according to embodiment 27 or 28, wherein Ring A is selected from phenyl, pyridinyl, pyrazolyl, 1H-pyrrolyl, indolinyl, and piperidinyl.

30. The compound, salt, or deuterated derivative according to any one of embodiments 27 to 29, wherein Ring A is phenyl.

31. The compound, salt, or deuterated derivative according to any one of embodiments 27 to 30, wherein Ring B is selected from C₆-C₁₀ aryl.

32. The compound, salt, or deuterated derivative according to any one of embodiments 27 to 31, wherein Ring B is phenyl.

33. The compound, salt, or deuterated derivative according to any one of embodiments 27 to 32, wherein W¹ is N and W² is N.

34. The compound, salt, or deuterated derivative according to any one of embodiments 27 to 33, wherein Z is selected from NR^{ZN} and C(R^{ZC}).

35. The compound, salt, or deuterated derivative according to any one of embodiments 27 to 34, wherein each R³ is independently selected from C₁-C₆ alkyl.

36. The compound, salt, or deuterated derivative according to any one of embodiments 27 to 35, wherein each R³ is methyl.

37. The compound, salt, or deuterated derivative according to any one of embodiments 27 to 34, wherein R³ is absent.

38. The compound, salt, or deuterated derivative according to any one of embodiments 27 to 37, wherein R⁴ is selected from hydrogen and methyl.

39. The compound, salt, or deuterated derivative according to any one of embodiments 27 to 38, wherein R⁴ is methyl.

40. The compound, salt, or deuterated derivative according to any one of embodiments 27 to 38, wherein R⁴ is hydrogen.

41. The compound, salt, or deuterated derivative according to any one of embodiments 27 to 40, wherein each R⁵ is independently selected from C₁-C₆ alkyl and C₁-C₆ alkoxy.

42. The compound, salt, or deuterated derivative according to any one of embodiments 27 to 41, wherein each R⁵ is independently selected from methyl,

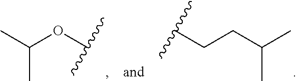

43. The compound, salt, or deuterated derivative according to any one of embodiments 27 to 42, wherein R^{ZN} is selected from hydrogen and R^{F}.

44. The compound, salt, or deuterated derivative according to any one of embodiments 27 to 43, wherein R^{ZN} is hydrogen.

45. The compound, salt, or deuterated derivative according to any one of embodiments 27 to 44, wherein R^{ZN} is R^{F}.

46. The compound, salt, or deuterated derivative according to any one of embodiments 27 to 45, wherein R^{ZC} is hydrogen, or two R^{ZC} are taken together to form an oxo group.

47. The compound, salt, or deuterated derivative according to any one of embodiments 27 to 46, wherein each R^{L1} is independently selected from hydrogen, C₁-C₉ alkyl optionally substituted with 1-3 groups independently selected from C₆-C₁₀ aryl, and R^{F}.

48. The compound, salt, or deuterated derivative according to any one of embodiments 27 to 47, wherein each R¹² is independently selected from hydrogen and R^{F}, or two R^{L2} on the same carbon atom are taken together to form an oxo group.

49. The compound, salt, or deuterated derivative according to any one of embodiments 27 to 48, wherein each R^{N} is independently selected from hydrogen and C₁-C₈ alkyl (optionally substituted with 1-3 groups independently selected from oxo, C₁-C₆ alkoxy, C₃-C₁₀ cycloalkyl, and C₆-C₁₀ aryl).

50. The compound, salt, or deuterated derivative according to any one of embodiments 27 to 49, wherein two R^{F} taken together with the atoms to which they are bonded form a group selected from:
C₆-C₁₀ aryl, and
3- to 11-membered heterocyclyl optionally substituted with 1-3 groups independently selected from:
   oxo,
   C₁-C₉ alkyl optionally substituted with 1-4 groups independently selected from:
      oxo,
      halogen,
      hydroxyl,
      N(R^{N})₂,
      C₁-C₆ alkoxy optionally substituted with 1-3 groups independently selected from C₆-C₁₀ aryl,
      C₆-C₁₀ aryl optionally substituted with 1-3 groups independently selected from hydroxyl, cyano, and C₁-C₆ alkyl,
      —(O)₀₋₁—(C₃-C₁₀ cycloalkyl) optionally substituted with 1-4 groups independently selected from N(R^{N})₂, C₁-C₆ alkyl (optionally substituted with 1-3 groups independently selected from oxo, hydroxyl, and C₁-C₆ alkoxy), and C₁-C₆ fluoroalkyl,
      3- to 10-membered heterocyclyl optionally substituted with 1-3 groups independently selected from oxo and C₁-C₆ alkyl, and
      5- to 10-membered heteroaryl optionally substituted with 1-3 groups independently selected from N(R^{N})₂, C₁-C₆ alkyl, and —O—(C₆-C₁₀ aryl),
   C₃-C₁₂ cycloalkyl optionally substituted with 1-4 groups independently selected from halogen and C₁-C₆ alkyl,
   C₆-C₁₀ aryl, and
   3- to 10-membered heterocyclyl.

51. A compound of Formula IIa:

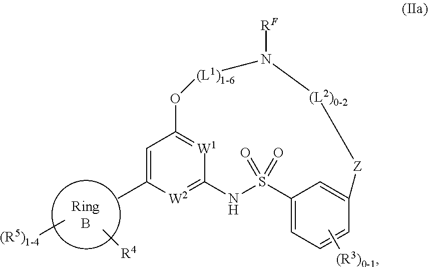

a tautomer thereof, a deuterated derivative of the compound or tautomer, or a pharmaceutically acceptable salt of any of the foregoing, wherein Ring B, W¹, W², Z, L¹, L², R³, R⁴, R⁵, and R^{F} are defined as according to embodiment 1.

52. The compound, salt, or deuterated derivative according to embodiment 51, wherein Ring B is selected from C₆-C₁₀ aryl.

53. The compound, salt, or deuterated derivative according to embodiment 51 or 52, wherein Ring B is phenyl.

54. The compound, salt, or deuterated derivative according to any one of embodiments 51 to 53, wherein W¹ is N and W² is N.

55. The compound, salt, or deuterated derivative according to any one of embodiments 51 to 54, wherein Z is selected from NR^{ZN} and C(R^{ZC}).

56. The compound, salt, or deuterated derivative according to any one of embodiments 51 to 55, wherein each R³ is independently selected from C₁-C₆ alkyl.

57. The compound, salt, or deuterated derivative according to any one of embodiments 51 to 56, wherein each R³ is methyl.

58. The compound, salt, or deuterated derivative according to any one of embodiments 51 to 55, wherein R³ is absent.

59. The compound, salt, or deuterated derivative according to any one of embodiments 51 to 58, wherein R⁴ is selected from hydrogen and methyl.

60. The compound, salt, or deuterated derivative according to any one of embodiments 51 to 59, wherein R⁴ is methyl.

61. The compound, salt, or deuterated derivative according to any one of embodiments 51 to 59, wherein R⁴ is hydrogen.

62. The compound, salt, or deuterated derivative according to any one of embodiments 51 to 61, wherein each R⁵ is independently selected from C₁-C₆ alkyl and C₁-C₆ alkoxy.

63. The compound, salt, or deuterated derivative according to any one of embodiments 51 to 62, wherein each R⁵ is independently selected from methyl,

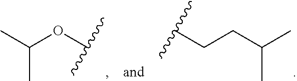

64. The compound, salt, or deuterated derivative according to any one of embodiments 51 to 63, wherein R^{ZN} is selected from hydrogen and R^{F}.

65. The compound, salt, or deuterated derivative according to any one of embodiments 51 to 64, wherein R^{ZN} is hydrogen.

66. The compound, salt, or deuterated derivative according to any one of embodiments 51 to 64, wherein R^{ZN} is R^{F}.

67. The compound, salt, or deuterated derivative according to any one of embodiments 51 to 66, wherein R^{ZC} is hydrogen, or two R^{ZC} are taken together to form an oxo group.

68. The compound, salt, or deuterated derivative according to any one of embodiments 51 to 67, wherein each R^{L1} is independently selected from hydrogen, C₁-C₉ alkyl optionally substituted with 1-3 groups independently selected from C₆-C₁₀ aryl, and R^{F}.

69. The compound, salt, or deuterated derivative according to any one of embodiments 51 to 68, wherein each R¹² is independently selected from hydrogen and R^{F}, or two R^{L2} on the same carbon atom are taken together to form an oxo group.

70. The compound, salt, or deuterated derivative according to any one of embodiments 51 to 69, wherein each R^{N} is independently selected from hydrogen and C₁-C₈ alkyl (optionally substituted with 1-3 groups independently selected from oxo, C₁-C₆ alkoxy, C₃-C₁₀ cycloalkyl, and C₆-C₁₀ aryl).

71. The compound, salt, or deuterated derivative according to any one of embodiments 51 to 70, wherein two R^{F} taken together with the atoms to which they are bonded form a group selected from:
C₆-C₁₀ aryl, and
3- to 11-membered heterocyclyl optionally substituted with 1-3 groups independently selected from:
   oxo,
   C₁-C₉ alkyl optionally substituted with 1-4 groups independently selected from:
      oxo,
      halogen,
      hydroxyl,
      N(R^{N})₂,
      C₁-C₆ alkoxy optionally substituted with 1-3 groups independently selected from C₆-C₁₀ aryl,
      C₆-C₁₀ aryl optionally substituted with 1-3 groups independently selected from hydroxyl, cyano, and C₁-C₆ alkyl,
      —(O)₀₋₁—(C₃-C₁₀ cycloalkyl) optionally substituted with 1-4 groups independently selected from N(R^{N})₂, C₁-C₆ alkyl (optionally substituted with 1-3 groups independently selected from oxo, hydroxyl, and C₁-C₆ alkoxy), and C₁-C₆ fluoroalkyl,
      3- to 10-membered heterocyclyl optionally substituted with 1-3 groups independently selected from oxo and C₁-C₆ alkyl, and
      5- to 10-membered heteroaryl optionally substituted with 1-3 groups independently selected from N(R^{N})₂, C₁-C₆ alkyl, and —O—(C₆-C₁₀ aryl),
   C₃-C₁₂ cycloalkyl optionally substituted with 1-4 groups independently selected from halogen and C₁-C₆ alkyl,
   C₆-C₁₀ aryl, and
   3- to 10-membered heterocyclyl.

72. A compound of Formula IIb:

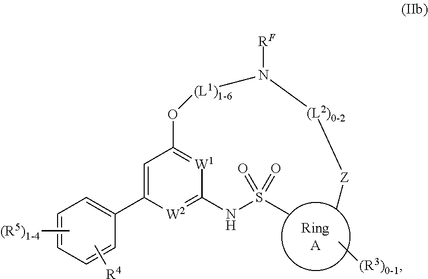

a tautomer thereof, a deuterated derivative of the compound or tautomer, or a pharmaceutically acceptable salt of any of the foregoing, wherein Ring A, W¹, W², Z, L¹, L², R³, R⁴, R⁵, and R^{F} are defined as according to embodiment 1.

73. The compound, salt, or deuterated derivative according to embodiment 72, wherein Ring A is selected from C₆-C₁₀ aryl, 3- to 10-membered heterocyclyl, and 5- to 10-membered heteroaryl.

74. The compound, salt, or deuterated derivative according to embodiment 72 or 73, wherein Ring A is selected from phenyl, pyridinyl, pyrazolyl, 1H-pyrrolyl, indolinyl, and piperidinyl.

75. The compound, salt, or deuterated derivative according to any one of embodiments 72 to 74, wherein Ring A is phenyl.

76. The compound, salt, or deuterated derivative according to any one of embodiments 72 to 75, wherein W¹ is N and W² is N.

77. The compound, salt, or deuterated derivative according to any one of embodiments 72 to 76, wherein Z is selected from NR^{ZN} and C(R^{ZC}).

78. The compound, salt, or deuterated derivative according to any one of embodiments 2 to 77, wherein each R³ is independently selected from C₁-C₆ alkyl.

79. The compound, salt, or deuterated derivative according to any one of embodiments 72 to 78, wherein each R³ is methyl.

80. The compound, salt, or deuterated derivative according to any one of embodiments 72 to 77, wherein R³ is absent.

81. The compound, salt, or deuterated derivative according to any one of embodiments 72 to 80, wherein R⁴ is selected from hydrogen and methyl.

82. The compound, salt, or deuterated derivative according to any one of embodiments 72 to 81, wherein R⁴ is methyl.

83. The compound, salt, or deuterated derivative according to any one of embodiments 72 to 81, wherein R⁴ is hydrogen.

84. The compound, salt, or deuterated derivative according to any one of embodiments 72 to 83, wherein each R⁵ is independently selected from C₁-C₆ alkyl and C₁-C₆ alkoxy.

85. The compound, salt, or deuterated derivative according to any one of embodiments 72 to 84, wherein each R⁵ is independently selected from methyl,

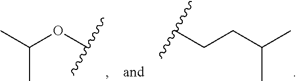

86. The compound, salt, or deuterated derivative according to any one of embodiments 72 to 85, wherein R^{ZN} is selected from hydrogen and R^{F}.

87. The compound, salt, or deuterated derivative according to any one of embodiments 72 to 86, wherein R^{ZN} is hydrogen.

88. The compound, salt, or deuterated derivative according to any one of embodiments 72 to 86, wherein R^{ZN} is R^{F}.

89. The compound, salt, or deuterated derivative according to any one of embodiments 72 to 88, wherein R^{ZC} is hydrogen, or two R^{ZC} are taken together to form an oxo group.

90. The compound, salt, or deuterated derivative according to any one of embodiments 72 to 89, wherein each R^{L1} is independently selected from hydrogen, C₁-C₉ alkyl optionally substituted with 1-3 groups independently selected from C₆-C₁₀ aryl, and R^{F}.

91. The compound, salt, or deuterated derivative according to any one of embodiments 72 to 90, wherein each R^{L2} is independently selected from hydrogen and R^{F}, or two R^{L2} on the same carbon atom are taken together to form an oxo group.

92. The compound, salt, or deuterated derivative according to any one of embodiments 72 to 91, wherein each R^{N} is independently selected from hydrogen and C₁-C₈ alkyl (optionally substituted with 1-3 groups independently selected from oxo, C₁-C₆ alkoxy, C₃-C₁₀ cycloalkyl, and C₆-C₁₀ aryl).

93. The compound, salt, or deuterated derivative according to any one of embodiments 72 to 92, wherein two R^{F} taken together with the atoms to which they are bonded form a group selected from:
C₆-C₁₀ aryl, and
3- to 11-membered heterocyclyl optionally substituted with 1-3 groups independently selected from:
   oxo,
   C₁-C₉ alkyl optionally substituted with 1-4 groups independently selected from:
      oxo,
      halogen,
      hydroxyl,
      N(R^{N})₂,
      C₁-C₆ alkoxy optionally substituted with 1-3 groups independently selected from C₆-C₁₀ aryl,
      C₆-C₁₀ aryl optionally substituted with 1-3 groups independently selected from hydroxyl, cyano, and C₁-C₆ alkyl,
      —(O)₀₋₁—(C₃-C₁₀ cycloalkyl) optionally substituted with 1-4 groups independently selected from N(R^{N})₂, C₁-C₆ alkyl (optionally substituted with 1-3 groups independently selected from oxo, hydroxyl, and C₁-C₆ alkoxy), and C₁-C₆ fluoroalkyl,
      3- to 10-membered heterocyclyl optionally substituted with 1-3 groups independently selected from oxo and C₁-C₆ alkyl, and
      5- to 10-membered heteroaryl optionally substituted with 1-3 groups independently selected from N(R^{N})₂, C₁-C₆ alkyl, and —O—(C₆-C₁₀ aryl),
   C₃-C₁₂ cycloalkyl optionally substituted with 1-4 groups independently selected from halogen and C₁-C₆ alkyl,
   C₆-C₁₀ aryl, and
   3- to 10-membered heterocyclyl.

94. A compound of Formula III:

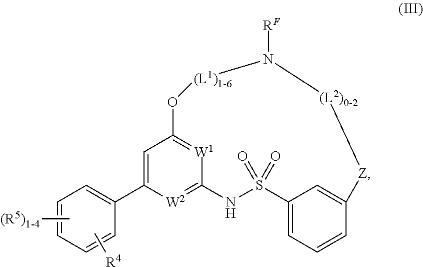

a tautomer thereof, a deuterated derivative of the compound or tautomer, or a pharmaceutically acceptable salt of any of the foregoing, wherein W¹, W², Z, L¹, L², R⁴, R⁵, and R^{F} are defined as according to embodiment 1.

95. The compound, salt, or deuterated derivative according to embodiment 94, wherein W¹ is N and W² is N.

96. The compound, salt, or deuterated derivative according to embodiment 94 or 95, wherein Z is selected from NR^{ZN} and C(R^{ZC})₂.

97. The compound, salt, or deuterated derivative according to any one of embodiments 94 to 96, wherein R⁴ is selected from hydrogen and methyl.

98. The compound, salt, or deuterated derivative according to any one of embodiments 94 to 97, wherein R⁴ is methyl.

99. The compound, salt, or deuterated derivative according to any one of embodiments 94 to 97, wherein R⁴ is hydrogen.

100. The compound, salt, or deuterated derivative according to any one of embodiments 94 to 99, wherein each R⁵ is independently selected from C₁-C₆ alkyl and C₁-C₆ alkoxy.

101. The compound, salt, or deuterated derivative according to any one of embodiments 94 to 100, wherein each R⁵ is independently selected from methyl,

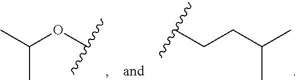

102. The compound, salt, or deuterated derivative according to any one of embodiments 94 to 101, wherein R^{ZN} is selected from hydrogen and R^{F}.

103. The compound, salt, or deuterated derivative according to any one of embodiments 94 to 102, wherein R^{ZN} is hydrogen.

104. The compound, salt, or deuterated derivative according to any one of embodiments 94 to 102, wherein R^{ZN} is R^{F}.

105. The compound, salt, or deuterated derivative according to any one of embodiments 94 to 104, wherein R^{ZC} is hydrogen, or two R^{ZC} are taken together to form an oxo group.

106. The compound, salt, or deuterated derivative according to any one of embodiments 94 to 105, wherein each R^{L1} is independently selected from hydrogen, C₁-C₉ alkyl optionally substituted with 1-3 groups independently selected from C₆-C₁₀ aryl, and R^{F}.

107. The compound, salt, or deuterated derivative according to any one of embodiments 94 to 106, wherein each R¹² is independently selected from hydrogen and R^{F}, or two R^{L2} on the same carbon atom are taken together to form an oxo group.

108. The compound, salt, or deuterated derivative according to any one of embodiments 94 to 107, wherein each R^{N} is independently selected from hydrogen and C₁-C₈ alkyl (optionally substituted with 1-3 groups independently selected from oxo, C₁-C₆ alkoxy, C₃-C₁₀ cycloalkyl, and C₆-C₁₀ aryl).

109. The compound, salt, or deuterated derivative according to any one of embodiments 94 to 108, wherein two R^{F} taken together with the atoms to which they are bonded form a group selected from:
C₆-C₁₀ aryl, and
3- to 11-membered heterocyclyl optionally substituted with 1-3 groups independently selected from:
   oxo,
   C₁-C₉ alkyl optionally substituted with 1-4 groups independently selected from:
      oxo,
      halogen,
      hydroxyl,
      N(R^{N})₂,
      C₁-C₆ alkoxy optionally substituted with 1-3 groups independently selected from C₆-C₁₀ aryl,
      C₆-C₁₀ aryl optionally substituted with 1-3 groups independently selected from hydroxyl, cyano, and C₁-C₆ alkyl,
      —(O)₀₋₁—(C₃-C₁₀ cycloalkyl) optionally substituted with 1-4 groups independently selected from N(R^{N})₂, C₁-C₆ alkyl (optionally substituted with 1-3 groups independently selected from oxo, hydroxyl, and C₁-C₆ alkoxy), and C₁-C₆ fluoroalkyl,
      3- to 10-membered heterocyclyl optionally substituted with 1-3 groups independently selected from oxo and C₁-C₆ alkyl, and
      5- to 10-membered heteroaryl optionally substituted with 1-3 groups independently selected from N(R^{N})₂, C₁-C₆ alkyl, and —O—(C₆-C₁₀ aryl),
   C₃-C₁₂ cycloalkyl optionally substituted with 1-4 groups independently selected from halogen and C₁-C₆ alkyl,
   C₆-C₁₀ aryl, and
   3- to 10-membered heterocyclyl.

110. A compound of Formula IV:

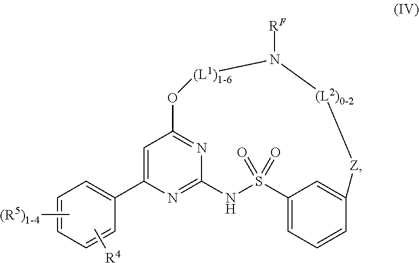

a tautomer thereof, a deuterated derivative of the compound or tautomer, or a pharmaceutically acceptable salt of any of the foregoing, wherein Z, L¹, L², R⁴, R⁵, and R^{F} are defined as according to embodiment 1.

111. The compound, salt, or deuterated derivative according to embodiment 110, wherein Z is selected from NR^{ZN} and C(R^{ZC}).

112. The compound, salt, or deuterated derivative according to embodiment 110 or 111, wherein R⁴ is selected from hydrogen and methyl.

113. The compound, salt, or deuterated derivative according to any one of embodiments 110 to 112, wherein R⁴ is methyl.

114. The compound, salt, or deuterated derivative according to any one of embodiments 110 to 112, wherein R⁴ is hydrogen.

115. The compound, salt, or deuterated derivative according to any one of embodiments 110 to 114, wherein each R⁵ is independently selected from C₁-C₆ alkyl and C₁-C₆ alkoxy.

116. The compound, salt, or deuterated derivative according to any one of embodiments 110 115, wherein each R⁵ is independently selected from methyl,

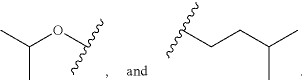

117. The compound, salt, or deuterated derivative according to any one of embodiments 110 to 116, wherein R^{ZN} is selected from hydrogen and R^{F}.

118. The compound, salt, or deuterated derivative according to any one of embodiments 110 to 117, wherein R^{ZN} is hydrogen.

119. The compound, salt, or deuterated derivative according to any one of embodiments 110 to 117, wherein R^{ZN} is R^{F}.

120. The compound, salt, or deuterated derivative according to any one of embodiments 110 to 119, wherein R^{ZC} is hydrogen, or two R^{ZC} are taken together to form an oxo group.

121. The compound, salt, or deuterated derivative according to any one of embodiments 110 to 120, wherein each R^{L1} is independently selected from hydrogen, C₁-C₉ alkyl optionally substituted with 1-3 groups independently selected from C₆-C₁₀ aryl, and R^{F}.

122. The compound, salt, or deuterated derivative according to any one of embodiments 110 to 121, wherein each R^{L2} is independently selected from hydrogen and R^{F}, or two R^{L2} on the same carbon atom are taken together to form an oxo group.

123. The compound, salt, or deuterated derivative according to any one of embodiments 110 to 122, wherein each R^{N} is independently selected from hydrogen and C₁-C₈ alkyl (optionally substituted with 1-3 groups independently selected from oxo, C₁-C₆ alkoxy, C₃-C₁₀ cycloalkyl, and C₆-C₁₀ aryl).

124. The compound, salt, or deuterated derivative according to any one of embodiments 110 to 123, wherein two R^{F} taken together with the atoms to which they are bonded form a group selected from:
C₆-C₁₀ aryl, and
3- to 11-membered heterocyclyl optionally substituted with 1-3 groups independently selected from:
   oxo,
   C₁-C₉ alkyl optionally substituted with 1-4 groups independently selected from:
      oxo,
      halogen,
      hydroxyl,
      N(R^{N})₂,
      C₁-C₆ alkoxy optionally substituted with 1-3 groups independently selected from C₆-C₁₀ aryl,
      C₆-C₁₀ aryl optionally substituted with 1-3 groups independently selected from hydroxyl, cyano, and C₁-C₆ alkyl,
      —(O)₀₋₁—(C₃-C₁₀ cycloalkyl) optionally substituted with 1-4 groups independently selected from N(R^{N})₂, C₁-C₆ alkyl (optionally substituted with 1-3 groups independently selected from oxo, hydroxyl, and C₁-C₆ alkoxy), and C₁-C₆ fluoroalkyl,
      3- to 10-membered heterocyclyl optionally substituted with 1-3 groups independently selected from oxo and C₁-C₆ alkyl, and
      5- to 10-membered heteroaryl optionally substituted with 1-3 groups independently selected from N(R^{N})₂, C₁-C₆ alkyl, and —O—(C₆-C₁₀ aryl),
   C₃-C₁₂ cycloalkyl optionally substituted with 1-4 groups independently selected from halogen and C₁-C₆ alkyl,
   C₆-C₁₀ aryl, and
   3- to 10-membered heterocyclyl.

125. A compound of Formula V:

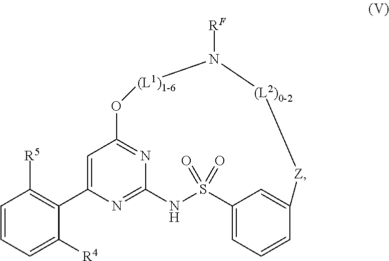

a tautomer thereof, a deuterated derivative of the compound or tautomer, or a pharmaceutically acceptable salt of any of the foregoing, wherein Z, L¹, L², R⁴, R⁵, and R^{F} are defined as according to embodiment 1.

126. The compound, salt, or deuterated derivative according to embodiment 125, wherein Z is selected from NR^{ZN} and C(R^{ZC})₂.

127. The compound, salt, or deuterated derivative according to embodiment 125 or 126, wherein R⁴ is selected from hydrogen and methyl.

128. The compound, salt, or deuterated derivative according to any one of embodiments 125 to 127, wherein R⁴ is methyl.

129. The compound, salt, or deuterated derivative according to any one of embodiments 125 to 127, wherein R⁴ is hydrogen.

130. The compound, salt, or deuterated derivative according to any one of embodiments 125 to 129, wherein each R⁵ is independently selected from C₁-C₆ alkyl and C₁-C₆ alkoxy.

131. The compound, salt, or deuterated derivative according to any one of embodiments 125 to 131, wherein each R⁵ is independently selected from methyl,

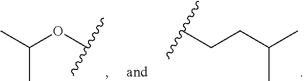

132. The compound, salt, or deuterated derivative according to any one of embodiments 125 to 131, wherein R^{ZN} is selected from hydrogen and R^{F}.

133. The compound, salt, or deuterated derivative according to any one of embodiments 125 to 132, wherein R^{ZN} is hydrogen.

134. The compound, salt, or deuterated derivative according to any one of embodiments 125 to 132, wherein R^{ZN} is R^{F}.

135. The compound, salt, or deuterated derivative according to any one of embodiments 125 to 134, wherein R^{ZC} is hydrogen, or two R^{ZC} are taken together to form an oxo group.

136. The compound, salt, or deuterated derivative according to any one of embodiments 125 to 135, wherein each R^{L1} is independently selected from hydrogen, C₁-C₉ alkyl optionally substituted with 1-3 groups independently selected from C₆-C₁₀ aryl, and R^{F}.

137. The compound, salt, or deuterated derivative according to any one of embodiments 125 to 136, wherein each R^{L2} is independently selected from hydrogen and R^{F}, or two R^{L2} on the same carbon atom are taken together to form an oxo group.

138. The compound, salt, or deuterated derivative according to any one of embodiments 125 to 137, wherein each R^{N} is independently selected from hydrogen and C₁-C₈ alkyl (optionally substituted with 1-3 groups independently selected from oxo, C₁-C₆ alkoxy, C₃-C₁₀ cycloalkyl, and C₆-C₁₀ aryl).

139. The compound, salt, or deuterated derivative according to any one of embodiments 125 to 138, wherein two R^{F} taken together with the atoms to which they are bonded form a group selected from:
C₆-C₁₀ aryl, and
3- to 11-membered heterocyclyl optionally substituted with 1-3 groups independently selected from:
   oxo,
   C₁-C₉ alkyl optionally substituted with 1-4 groups independently selected from:
      oxo,
      halogen,
      hydroxyl,
      N(R^{N})₂,
      C₁-C₆ alkoxy optionally substituted with 1-3 groups independently selected from C₆-C₁₀ aryl,
      C₆-C₁₀ aryl optionally substituted with 1-3 groups independently selected from hydroxyl, cyano, and C₁-C₆ alkyl,
      —(O)₀₋₁—(C₃-C₁₀ cycloalkyl) optionally substituted with 1-4 groups independently selected from N(R^{N})₂, C₁-C₆ alkyl (optionally substituted with 1-3 groups independently selected from oxo, hydroxyl, and C₁-C₆ alkoxy), and C₁-C₆ fluoroalkyl,
      3- to 10-membered heterocyclyl optionally substituted with 1-3 groups independently selected from oxo and C₁-C₆ alkyl, and
      5- to 10-membered heteroaryl optionally substituted with 1-3 groups independently selected from N(R^{N})₂, C₁-C₆ alkyl, and —O—(C₆-C₁₀ aryl),
   C₃-C₁₂ cycloalkyl optionally substituted with 1-4 groups independently selected from halogen and C₁-C₆ alkyl,
   C₆-C₁₀ aryl, and
   3- to 10-membered heterocyclyl.

140. A compound of Formula VI:

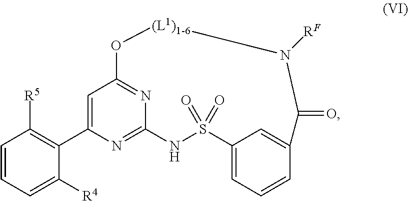

a tautomer thereof, a deuterated derivative of the compound or tautomer, or a pharmaceutically acceptable salt of any of the foregoing, wherein L¹, R⁴, R⁵, and R^{F} are defined as according to embodiment 1.

141. The compound, salt, or deuterated derivative according to embodiment 140, wherein R⁴ is selected from hydrogen and methyl.

142. The compound, salt, or deuterated derivative according to embodiment 140 or 141, wherein R⁴ is methyl.

143. The compound, salt, or deuterated derivative according to embodiment 140 or 141, wherein R⁴ is hydrogen.

144. The compound, salt, or deuterated derivative according to any one of embodiments 140 to 143, wherein each R⁵ is independently selected from C₁-C₆ alkyl and C₁-C₆ alkoxy.

145. The compound, salt, or deuterated derivative according to any one of embodiments 140 to 144, wherein each R⁵ is independently selected from methyl,

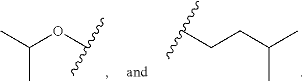

146. The compound, salt, or deuterated derivative according to any one of embodiments 140 to 145, wherein each R^{L1} is independently selected from hydrogen, C₁-C₉ alkyl optionally substituted with 1-3 groups independently selected from C₆-C₁₀ aryl, and R^{F}.

147. The compound, salt, or deuterated derivative according to any one of embodiments 140 to 146, wherein each R^{N} is independently selected from hydrogen and C₁-C₈ alkyl (optionally substituted with 1-3 groups independently selected from oxo, C₁-C₆ alkoxy, C₃-C₁₀ cycloalkyl, and C₆-C₁₀ aryl).

148. The compound, salt, or deuterated derivative according to any one of embodiments 140 to 147, wherein two R^{F} taken together with the atoms to which they are bonded form a group selected from:
C₆-C₁₀ aryl, and
3- to 11-membered heterocyclyl optionally substituted with 1-3 groups independently selected from:
   oxo,
   C₁-C₉ alkyl optionally substituted with 1-4 groups independently selected from:
      oxo,
      halogen,
      hydroxyl,
      N(R^{N})₂,
      C₁-C₆ alkoxy optionally substituted with 1-3 groups independently selected from C₆-C₁₀ aryl,
      C₆-C₁₀ aryl optionally substituted with 1-3 groups independently selected from hydroxyl, cyano, and C₁-C₆ alkyl,
      —(O)₀₋₁—(C₃-C₁₀ cycloalkyl) optionally substituted with 1-4 groups independently selected from N(R^{N})₂, C₁-C₆ alkyl (optionally substituted with 1-3 groups independently selected from oxo, hydroxyl, and C₁-C₆ alkoxy), and C₁-C₆ fluoroalkyl,
      3- to 10-membered heterocyclyl optionally substituted with 1-3 groups independently selected from oxo and C₁-C₆ alkyl, and
      5- to 10-membered heteroaryl optionally substituted with 1-3 groups independently selected from N(R^{N})₂, C₁-C₆ alkyl, and —O—(C₆-C₁₀ aryl),
   C₃-C₁₂ cycloalkyl optionally substituted with 1-4 groups independently selected from halogen and C₁-C₆ alkyl,
   C₆-C₁₀ aryl, and
   3- to 10-membered heterocyclyl.

149. The compound, tautomer, deuterated derivative, or pharmaceutically acceptable salt according to any one of embodiments 1 to 148, selected from compounds of any one of Formulae I, Ia, IIa, IIb, III, IV, V, Va, Vb, and VI, tautomers thereof, deuterated derivatives of those compounds and tautomers, and pharmaceutically acceptable salts of any of the foregoing.

150. The compound, tautomer, deuterated derivative, or pharmaceutically acceptable salt according to any one of embodiments 1 to 149, selected from Compounds 1-371 (Tables 13, 14, and 15), Compounds 372-385 (Table 12), Compounds 386-426 (Table 24), tautomers thereof, deuterated derivatives of those compounds and tautomers, and pharmaceutically acceptable salts of any of the foregoing.

151. A pharmaceutical composition comprising the compound, tautomer, deuterated derivative, or pharmaceutically acceptable salt according to any one of embodiments 1 to 150, and a pharmaceutically acceptable carrier.

152. The pharmaceutical composition of embodiment 151, further comprising one or more additional therapeutic agent(s).

153. The pharmaceutical composition of embodiment 152, wherein the one or more additional therapeutic agent(s) is selected from mucolytic agents, bronchodilators, antibiotics, anti-infective agents, and anti-inflammatory agents.

154. The pharmaceutical composition of embodiment 152, wherein the one or more additional therapeutic agent(s) is an antibiotic selected from tobramycin, including tobramycin inhaled powder (TIP), azithromycin, aztreonam, including the aerosolized form of aztreonam, amikacin, including liposomal formulations thereof, ciprofloxacin, including formulations thereof suitable for administration by inhalation, levoflaxacin, including aerosolized formulations thereof, and combinations of two antibiotics, e.g., fosfomycin and tobramycin.

155. The pharmaceutical composition of embodiment 152, wherein the one or more additional therapeutic agent(s) is a CFTR modulator.

156. The pharmaceutical composition of embodiment 155, wherein the CFTR modulator is a potentiator.

157. The pharmaceutical composition of embodiment 155, wherein the CFTR modulator is a corrector.

158. The pharmaceutical composition of embodiment 156, comprising both a CFTR potentiator and a CFTR corrector.

159. The pharmaceutical composition of embodiment 155 or embodiment 158, wherein the CFTR potentiator is selected from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.

160. The pharmaceutical composition of embodiment 157 or embodiment 158, wherein the CFTR corrector is selected from tezacaftor and lumacaftor.

161. The pharmaceutical composition of embodiment 152, wherein the composition comprises ivacaftor and tezacaftor.

162. The pharmaceutical composition of embodiment 152, wherein the composition comprises deutivacaftor and tezacaftor.

163. The pharmaceutical composition of embodiment 152, wherein the composition comprises (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and tezacaftor.

164. The pharmaceutical composition of embodiment 152, wherein the composition comprises ivacaftor and lumacaftor.

165. The pharmaceutical composition of embodiment 152, wherein the composition comprises deutivacaftor and lumacaftor.

166. The pharmaceutical composition of embodiment 152, wherein the composition comprises (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and lumacaftor.

167. A method of treating cystic fibrosis comprising administering to a patient in need thereof the compound, tautomer, deuterated derivative, or pharmaceutically acceptable salt according to any one of embodiments 1 to 150, or a pharmaceutical composition according to any one of embodiments 151 to 166.

168. The method of embodiment 167, further comprising administering to the patient one or more additional therapeutic agents prior to, concurrent with, or subsequent to the compound, tautomer, deuterated derivative, or pharmaceutically acceptable salt according to any one of embodiments 1 to 150 or the pharmaceutical composition according to embodiment 151.

169. The method of embodiment 168, wherein the one or more additional therapeutic agents is (are) selected from CFTR modulators.

170. The method of embodiment 169, wherein the CFTR modulator is a potentiator.

171. The method of embodiment 169, wherein the CFTR modulator is a corrector.

172. The method of embodiment 169, comprising administration of both a CFTR potentiator and an additional CFTR corrector.

173. The method of embodiment 170 or embodiment 172, wherein the CFTR potentiator is selected from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.

174. The method of embodiment 171 or embodiment 172, wherein the CFTR corrector is selected from tezacaftor and lumacaftor.

175. The method of embodiment 169, comprising administration of ivacaftor and tezacaftor.

176. The method of embodiment 169, comprising administration of deutivacaftor and tezacaftor.

177. The method of embodiment 169, comprising administration of (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and tezacaftor.

178. The method of embodiment 169, comprising administration of ivacaftor and lumacaftor.

179. The method of embodiment 169, comprising administration of deutivacaftor and lumacaftor.

180. The method of embodiment 169, comprising administration of (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and lumacaftor.

181. The compound, tautomer, deuterated derivative, or pharmaceutically acceptable salt according to any one of embodiments 1 to 150, or the pharmaceutical composition according to any one of embodiments 151 to 166 for use in the treatment of cystic fibrosis.

182. The compound, tautomer, deuterated derivative, or pharmaceutically acceptable salt according to any one of embodiments 1 to 150, or the pharmaceutical composition according to any one of embodiments 151 to 166 for use in the manufacture of a medicament for the treatment of cystic fibrosis.

183. A compound selected from Compounds 1-426, tautomers thereof, deuterated derivatives of those compounds and tautomers, and pharmaceutically acceptable salts of any of the foregoing.

184. A deuterated derivative of a compound selected from Compounds 1-426.

185. A pharmaceutically acceptable salt of a compound selected from Compounds 1-426.

186. A compound selected from Compounds 1-426.

187. A pharmaceutical composition comprising a compound selected from Compounds 1-426, tautomers thereof, deuterated derivatives of those compounds and tautomers, and pharmaceutically acceptable salts of any of the foregoing and a pharmaceutically acceptable carrier.

188. A pharmaceutical composition comprising a deuterated derivative of a compound selected from Compounds 1-426 and a pharmaceutically acceptable carrier.

189. A pharmaceutical composition comprising a pharmaceutically acceptable salt of a compound selected from Compounds 1-426 and a pharmaceutically acceptable carrier.

190. A pharmaceutical composition comprising a compound selected from Compounds 1-426 and a pharmaceutically acceptable carrier.

191. A pharmaceutical composition comprising (a) a compound selected from Compounds 1-426, tautomers thereof, deuterated derivatives of those compounds and tautomers, and pharmaceutically acceptable salts of any of the foregoing; (b) a CFTR potentiator; and (c) a pharmaceutically acceptable carrier.

192. A pharmaceutical composition composition comprising (a) a deuterated derivative of a compound selected from Compounds 1-426; (b) a CFTR potentiator; and (c) a pharmaceutically acceptable carrier.

193. A pharmaceutical comprising (a) a pharmaceutically acceptable salt of a compound selected from Compounds 1-426; (b) a CFTR potentiator; and (c) a pharmaceutically acceptable carrier.

194. A pharmaceutical composition comprising (a) a compound selected from Compounds 1-426; (b) a CFTR potentiator; and (c) a pharmaceutically acceptable carrier.

195. A pharmaceutical composition comprising (a) a compound selected from Compounds 1-426, tautomers thereof, deuterated derivatives of those compounds and tautomers, and pharmaceutically acceptable salts of any of the foregoing; (b) an additional CFTR corrector; and (c) a pharmaceutically acceptable carrier.

196. A pharmaceutical composition comprising (a) a deuterated derivative of a compound selected from Compounds 1-426; (b) an additional CFTR corrector; and (c) a pharmaceutically acceptable carrier.

197. A pharmaceutical composition comprising (a) a pharmaceutically acceptable salt of a compound selected from Compounds 1-426; (b) an additional CFTR corrector; and (c) a pharmaceutically acceptable carrier.

198. A pharmaceutical composition comprising (a) a compound selected from Compounds 1-426; (b) an additional CFTR corrector; and (c) a pharmaceutically acceptable carrier.

199. A pharmaceutical composition comprising (a) a compound selected from Compounds 1-426, tautomers thereof, deuterated derivatives of those compounds and tautomers, and pharmaceutically acceptable salts of any of the foregoing; (b) an additional CFTR corrector; (c) a CRTR potentiator; and (d) a pharmaceutically acceptable carrier.

200. A pharmaceutical composition comprising (a) a deuterated derivative of a compound selected from Compounds 1-426; (b) an additional CFTR corrector; (c) a CFTR potentiator; and (d) a pharmaceutically acceptable carrier.

201. A pharmaceutical composition comprising (a) a pharmaceutically acceptable salt of a compound selected from Compounds 1-426; (b) an additional CFTR corrector; (c) a CFTR potentiator; and (d) a pharmaceutically acceptable carrier.

202. A pharmaceutical composition comprising (a) a compound selected from Compounds 1-426; (b) an additional CFTR corrector; (c) a CFTR potentiator; and (d) a pharmaceutically acceptable carrier.

203. A compound selected from Compounds 1-426, tautomers thereof, deuterated derivatives of those compounds and tautomers, and pharmaceutically acceptable salts of any of the foregoing for use in a method of treating cystic fibrosis.

204. A deuterated derivative of a compound selected from Compounds 1-426 for use in a method of treating cystic fibrosis.

205. A pharmaceutically acceptable salt of a compound selected from Compounds 1-426 for use in a method of treating cystic fibrosis.

206. A compound selected from Compounds 1-426 for use in a method of treating cystic fibrosis.

207. A pharmaceutical composition comprising a compound selected from Compounds 1-426, tautomers thereof, deuterated derivatives of those compounds and tautomers, and pharmaceutically acceptable salts of any of the foregoing and a pharmaceutically acceptable carrier for use in a method of treating cystic fibrosis.

208. A pharmaceutical composition comprising a deuterated derivative of a compound selected from Compounds 1-426 and a pharmaceutically acceptable carrier for use in a method of treating cystic fibrosis.

209. A pharmaceutical composition comprising a pharmaceutically acceptable salt of a compound selected from Compounds 1-426 and a pharmaceutically acceptable carrier for use in a method of treating cystic fibrosis.

210. A pharmaceutical composition comprising a compound selected from Compounds 1-426 and a pharmaceutically acceptable carrier for use in a method of treating cystic fibrosis.

211. A pharmaceutical composition comprising (a) a compound selected from Compounds 1-426, tautomers thereof, deuterated derivatives of those compounds and tautomers, and pharmaceutically acceptable salts of any of the foregoing; (b) a CFTR potentiator; and (c) a pharmaceutically acceptable carrier for use in a method of treating cystic fibrosis.

212. A pharmaceutical comprising (a) a deuterated derivative of a compound selected from Compounds 1-426; (b) a CFTR potentiator; and (c) a pharmaceutically acceptable carrier for use in a method of treating cystic fibrosis.

213. A pharmaceutical composition comprising (a) a pharmaceutically acceptable salt of a compound selected from Compounds 1-426; (b) a CFTR potentiator; and (c) a pharmaceutically acceptable carrier for use in a method of treating cystic fibrosis.

214. A pharmaceutical composition comprising (a) a compound selected from Compounds 1-426; (b) a CFTR potentiator; and (c) a pharmaceutically acceptable carrier.

215. A pharmaceutical composition comprising (a) a compound selected from Compounds 1-426, tautomers thereof, deuterated derivatives of those compounds and tautomers, and pharmaceutically acceptable salts of any of the foregoing; (b) an additional CFTR corrector; and (c) a pharmaceutically acceptable carrier for use in a method of treating cystic fibrosis.

216. A pharmaceutical composition comprising (a) a deuterated derivative of a compound selected from Compounds 1-426; (b) an additional CFTR corrector; and (c) a pharmaceutically acceptable carrier for use in a method of treating cystic fibrosis.

217. A pharmaceutical composition comprising (a) a pharmaceutically acceptable salt of a compound selected from Compounds 1-426; (b) an additional CFTR corrector; and (c) a pharmaceutically acceptable carrier for use in a method of treating cystic fibrosis.

218. A pharmaceutical composition comprising (a) a compound selected from Compounds 1-426; (b) an additional CFTR corrector; and (c) a pharmaceutically acceptable carrier for use in a method of treating cystic fibrosis.

219. A pharmaceutical composition comprising (a) a compound selected from Compounds 1-426, tautomers thereof, deuterated derivatives of those compounds and tautomers, and pharmaceutically acceptable salts of any of the foregoing; (b) an additional CFTR corrector; (c) a CRTR potentiator; and (d) a pharmaceutically acceptable carrier for use in a method of treating cystic fibrosis.

220. A pharmaceutical composition comprising (a) a deuterated derivative of a compound selected from Compounds 1-426; (b) an additional CFTR corrector; (c) a CFTR potentiator; and (d) a pharmaceutically acceptable carrier for use in a method of treating cystic fibrosis.

221. A pharmaceutical composition comprising (a) a pharmaceutically acceptable salt of a compound selected from Compounds 1-426; (b) an additional CFTR corrector; (c) a CFTR potentiator; and (d) a pharmaceutically acceptable carrier for use in a method of treating cystic fibrosis.

222. A pharmaceutical composition comprising (a) a compound selected from Compounds 1-426; (b) an additional CFTR corrector; (c) a CFTR potentiator; and (d) a pharmaceutically acceptable carrier for use in a method of treating cystic fibrosis.

### EXAMPLES

### I. Abbreviation List

ACN: Acetonitrile
Boc anhydride ((Boc)₂O): Di-tert-butyl dicarbonate
CDCl₃: Chloroform-d CDI: Carbonyl diimidazole
CDMT: 2-Chloro-4,6-dimethoxy-1,3,5-triazine
CH₂Cl₂: Dichloromethane
CH₃CN: Acetonitrile
COMU: (1-Cyano-2-ethoxy-2-oxoethylidenaminooxy)dimethylamino-morpholino-carbenium
hexafluorophosphate
Cmpd: Compound
DABCO: 1,4-Diazabicyclo[2.2.2]octane
DBU: 1,8-Diazabicyclo(5.4.0)undec-7-ene
DCE: 1,2-Dichloroethane
DCM: Dichloromethane
DI: Deionized
DIAD: Diisopropyl azodicarboxylate
DIEA: (DIPEA, DiPEA): N,N-diisopropylethylamine
DMA: N,N-Dimethylacetamide
DMAP: 4-Dimethylaminopyridine
DMF: N,N-Dimethylformamide
DMSO: Dimethyl sulfoxide
DMP: Dess-Martin periodinane
EA: Ethyl acetate
EDC: 1-Ethyl-3-(3-dimethylaminopropyl)carbodiimide
ELSD: Evaporative light scattering detector
ESI-MS: Electrospray ionization mass spectrometry
EtOAc: Ethyl acetate
EtOH: Ethanol
GC: Gas chromatography
Grubbs 1^{st} Generation catalyst: Dichloro(benzylidene)bis(tricyclohexylphosphine)ruthenium(II)
Grubbs 2^{nd} Generation catalyst: [1,3-Bis(2,4,6-trimethylphenyl)imidazolidin-2-ylidene]-dichloro-[(2-isopropoxyphenyl)methylene]ruthenium
HATU: 1-[Bis(dimethylamino)methylene]-1H-1,2,3-triazolo[4,5-b]pyridinium 3-oxid hexafluorophosphate
HPLC: High-performance liquid chromatography
Hoveyda-Grubbs 2^{nd} Generation catalyst: (1,3-Bis-(2,4,6-trimethylphenyl)-2-imidazolidinylidene)dichloro(o-isopropoxyphenylmethylene)ruthenium, Dichloro[1,3-bis(2,4,6-trimethylphenyl)-2-imidazolidinylidene](2-isopropoxyphenylmethylene)ruthenium(II)
IPA: Isopropanol
KHSO₄: Potassium bisulfate
LC: Liquid chromatography
LCMS: Liquid chromatography mass spectrometry
LCMS Met.: LCMS method
LCMS Rt: LCMS retention time
LDA: Lithium diisopropylamide
LiOH: Lithium hydroxide
MeCN: Acetonitrile
MeOH: Methanol
MgSO₄: Magnesium sulfate
MTBE: Methyl tert-butyl ether
MeTHF or 2-MeTHF: 2-Methyltetrahydrofuran
NaHCO₃: Sodium bicarbonate
NaOH: Sodium hydroxide
NMP: N-Methyl-2-pyrrolidone
NMM: N-Methylmorpholine
Pd/C: Palladium on carbon
Pd₂(dba)₃: Tris(dibenzylideneacetone)dipalladium(O)
Pd(dppf)Cl₂: [1,1′-Bis(diphenylphosphino)ferrocene]dichloropalladium(II)
Pd(OAc)₂: Palladium(II) acetate
PTFE: Polytetrafluoroethylene
rt, RT: Room temperature
RuPhos: 2-Dicyclohexylphosphino-2′,6′-diisopropoxybiphenyl
SFC: Supercritical fluid chromatography
TBAI: Tetrabutylammonium iodide
TEA: Triethylamine
TFA: Trifluoroacetic acid
THF: Tetrahydrofuran
TLC: Thin layer chromatography
TMS: Trimethylsilyl
TMSCl: Trimethylsilyl chloride
T3P: Propanephosphonic acid anhydride
UPLC: Ultra Performance Liquid Chromatography
XANTPHOS: 4,5-Bis(diphenylphosphino)-9,9-dimethylxanthene
XPhos: 2-Dicyclohexylphosphino-2′,4′,6′-triisopropylbiphenyl

### II. General Methods

Reagents and starting materials were obtained by commercial sources unless otherwise stated and were used without purification.

Proton and carbon NMR spectra were acquired on either a Bruker Biospin DRX 400 MHz FTNMR spectrometer operating at a ¹H and ¹³C resonant frequency of 400 and 100 MHz respectively, or on a 300 MHz NMR spectrometer. One dimensional proton and carbon spectra were acquired using a broadband observe (BBFO) probe with 20 Hz sample rotation at 0.1834 and 0.9083 Hz/Pt digital resolution respectively. All proton and carbon spectra were acquired with temperature control at 30° C. using standard, previously published pulse sequences and routine processing parameters.

NMR (1D & 2D) spectra were also recorded on a Bruker AVNEO 400 MHz spectrometer operating at 400 MHz and 100 MHz respectively equipped with a 5 mm multinuclear Iprobe.

NMR spectra were also recorded on a Varian Mercury NMR instrument at 300 MHz for ¹H using a 45 degree pulse angle, a spectral width of 4800 Hz and 28860 points of acquisition. FID were zero-filled to 32 k points and a line broadening of 0.3 Hz was applied before Fourier transform. ¹⁹F NMR spectra were recorded at 282 MHz using a 30 degree pulse angle, a spectral width of 100 kHz and 59202 points were acquired. FID were zero-filled to 64 k points and a line broadening of 0.5 Hz was applied before Fourier transform.

NMR spectra were also recorded on a Bruker Avance III HD NMR instrument at 400 MHz for ¹H using a 30 degree pulse angle, a spectral width of 8000 Hz and 128 k points of acquisition. FID were zero-filled to 256 k points and a line broadening of 0.3 Hz was applied before Fourier transform. 19F NMR spectra were recorded at 377 MHz using a 30 deg pulse angle, a spectral width of 89286 Hz and 128 k points were acquired. FID were zero-filled to 256 k points and a line broadening of 0.3 Hz was applied before Fourier transform.

NMR spectra were also recorded on a Bruker AC 250 MHz instrument equipped with a: 5 mm QNP(H1/C13/F19/P31) probe (type: 250-SB, s #23055/0020) or on a Varian 500 MHz instrument equipped with a ID PFG, 5 mm, 50-202/500 MHz probe (model/part #99337300).

Final purity of compounds was determined by reversed phase UPLC using an Acquity UPLC BEH C₁₈ column (50×2.1 mm, 1.7 m particle) made by Waters (pn: 186002350), and a dual gradient run from 1-99% mobile phase B over 3.0 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C. Final purity was calculated by averaging the area under the curve (AUC) of two UV traces (220 nm, 254 nm). Low-resolution mass spectra were reported as [M+1]⁺ species obtained using a single quadrupole mass spectrometer equipped with an electrospray ionization (ESI) source capable of achieving a mass accuracy of 0.1 Da and a minimum resolution of 1000 (no units on resolution) across the detection range. Optical purity of methyl (2S)-2,4-dimethyl-4-nitro-pentanoate was determined using chiral gas chromatography (GC) analysis on an Agilent 7890A/MSD 5975C instrument, using a Restek Rt-βDEXcst (30 m×0.25 mm×0.25 μm_df) column, with a 2.0 mL/min flow rate (H2 carrier gas), at an injection temperature of 220° C. and an oven temperature of 120° C., 15 minutes.

### III. General UPLC/HPLC Analytical Methods

LC method A: Analytical reverse phase UPLC using an Acquity UPLC BEH Cis column (50×2.1 mm, 1.7 m particle) made by Waters (pn: 186002350), and a dual gradient run from 1-99% mobile phase B over 3.0 minutes. Mobile phase A=H₂O (0.05% CF₃CO₂H). Mobile phase B=CH₃CN (0.035% CF₃CO₂H). Flow rate=1.2 mL/min, injection volume=1.5 L, and column temperature=60° C.

LC method B: Reverse phase HPLC using a Kinetex C₁₈ column (50×3.0 mm) and a dual gradient run from 5-100% mobile phase B over 6 minutes. Mobile phase A=H₂O (0.1% CF₃CO₂H). Mobile phase B=CH₃CN (0.1% CF₃CO₂H). Flow rate=1.5 mL/min, injection volume=2 μL, and column temperature=60° C.

LC method C: Kinetex C₁₈ 4.6×50 mm 2.6 μm. Temp: 45° C., Flow: 2.0 mL/minutes, Run Time: 3 minutes. Mobile phase: Initial 95% water (0.1% formic acid) and 5% acetonitrile (0.1% formic acid) linear gradient to 95% acetonitrile (0.1% formic acid) for 2.0 minutes then hold at 95% acetonitrile (0.1% formic acid) for 1.0 minute.

LC method D: Acquity UPLC BEH C₁₈ column (30×2.1 mm, 1.7 m particle) made by Waters (pn: 186002349), and a dual gradient run from 1-99% mobile phase B over 1.0 minute. Mobile phase A=H₂O (0.05% CF₃CO₂H). Mobile phase B=CH₃CN (0.035% CF₃CO₂H). Flow rate=1.5 mL/min, injection volume=1.5 μL, and column temperature=60° C.

LC method G: Symmetry, 4.6×75 mm 3.5 μm. Temp: 45° C., Flow: 2.0 mL/minutes, Run Time: 8 min. Mobile Phase: Initial 95% H₂O (0.1% Formic Acid) and 5% CH₃CN (0.1% FA) linear gradient to 95% CH₃CN (0.1% formic acid) for 6.0 minutes then hold at 95% CH₃CN (0.1% formic acid) for 2.0 minutes.

LC method H: Kinetex C₁₈ 4.6×50 mm 2.6 um. Temp: 45° C., Flow: 2.0 mL/min, Run Time: 6 minutes. Mobile Phase: Initial 95% H₂O (0.1% Formic Acid) and 5% CH₃CN (0.1% FA) linear gradient to 95% CH₃CN (0.1% FA) for 4.0 minutes then hold at 95% CH₃CN (0.1% FA) for 2.0 minutes.

LC method I: Acquity UPLC BEH C₁₈ column (50×2.1 mm, 1.7 m particle) made by Waters (pn:186002350), and a dual gradient run from 1-99% mobile phase B over 5.0 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

LC method J: Reverse phase UPLC using an Acquity UPLC BEH C₁₈ column (50×2.1 mm, 1.7 m particle) made by Waters (pn: 186002350), and a dual gradient run from 1-99% mobile phase B over 2.9 minutes. Mobile phase A=H₂O (0.05% NH₄HCO₂). Mobile phase B=CH₃CN. Flow rate=1.2 mL/min, injection volume=1.5 μL, and column temperature=60° C.

LC method K: Kinetex Polar C₁₈ 3.0×50 mm 2.6 μm, 3 min, 5-95% ACN in H₂O (0.1% Formic Acid) 1.2 mL/minutes.

LC method M: Poroshell 120 EC-C₁₈ 3.0×50 mm 2.7 μM, Temp: 45° C., Flow: 2.0 ml/min, Run Time: 6 minutes. Mobile Phase Conditions: Initial 95% H₂O (0.1% FA) and 5% CH₃CN (0.1% FA) linear gradient to 95% CH₃CN (0.1% FA) for 4.0 minutes then hold at 95% CH₃CN (0.1% FA) for 2.0 minutes.

LC method N: Kinetex EVO C₁₈ 4.6×50 mm 2.6 m, Temp: 45° C., Flow: 2.0 mL/min, Run Time: 4 minutes. Mobile Phase: Initial 95% H₂O (0.1% Formic Acid) and 5% CH₃CN (0.1% FA) linear gradient to 95% CH₃CN (0.1% FA) for 2.0 minutes then hold at 95% CH₃CN (0.1% FA) for 2.0 minutes.

LC method O: Zorbax C₁₈ 4.6×50 mm 3.5 μM, 2.0 mL/min, 95% H₂O (0.1% formic acid)+5% CH₃CN (0.1% FA) to 95% CH₃CN (0.1% FA) gradient (2.0 minutes) then hold at 95% CH₃CN (0.1% FA) for 1.0 minutes.

LC method P: Poroshell 120 EC-C18 3.0×50 mm 2.7 μM, Temp:45° C., Flow: 1.5 mL/min, Run Time: 3 minutes. Mobile phase conditions: Initial. 95% H₂O (0.1% Formic Acid) and 5% CH₃CN (0.1% FA) linear gradient to 95% CH₃CN (0.1% FA) for 1.5 min then hold at 95% CH₃CN (0.1% FA) for 1.5 minutes.

LC method Q: Reversed phase UPLC using an Acquity UPLC BEH C₁₈ column (50×2.1 mm, 1.7 m particle) made by Waters (pn: 186002350), and a dual gradient run from 30-99% mobile phase B over 2.9 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

LC method S: Merckmillipore Chromolith SpeedROD C₁₈ column (50×4.6 mm) and a dual gradient run from 5-100% mobile phase B over 12 minutes. Mobile phase A=water (0.1% CF₃CO₂H). Mobile phase B=acetonitrile (0.1% CF₃CO₂H).

LC method T: Merckmillipore Chromolith SpeedROD C₁₈ column (50×4.6 mm) and a dual gradient run from 5-100% mobile phase B over 6 minutes. Mobile phase A=water (0.1% CF₃CO₂H). Mobile phase B=acetonitrile (0.1% CF₃CO₂H).

LC method U: Kinetex Polar C₁₈ 3.0×50 mm 2.6 μm, 6 minutes, 5-95% ACN in H₂O (0.1% Formic Acid) 1.2 mL/min.

LC method W: water Cortex 2.7μ C₁₈ (3.0 mm×50 mm), Temp: 55° C.; Flow: 1.2 mL/min; mobile phase: 100% water with 0.1% trifluoroacetic (TFA) acid then 100% acetonitrile with 0.1% TFA acid, grad:5% to 100% B over 4 min, with stay at 100% B for 0.5 minutes, equilibration to 5% B over 1.5 minutes.

### IV. Synthesis of Common Intermediates

### Example A: Preparation of 3-[[4-Chloro-6-(2,6-dimethylphenyl)pyrimidin-2-yl]sulfamoyl]benzoic acid

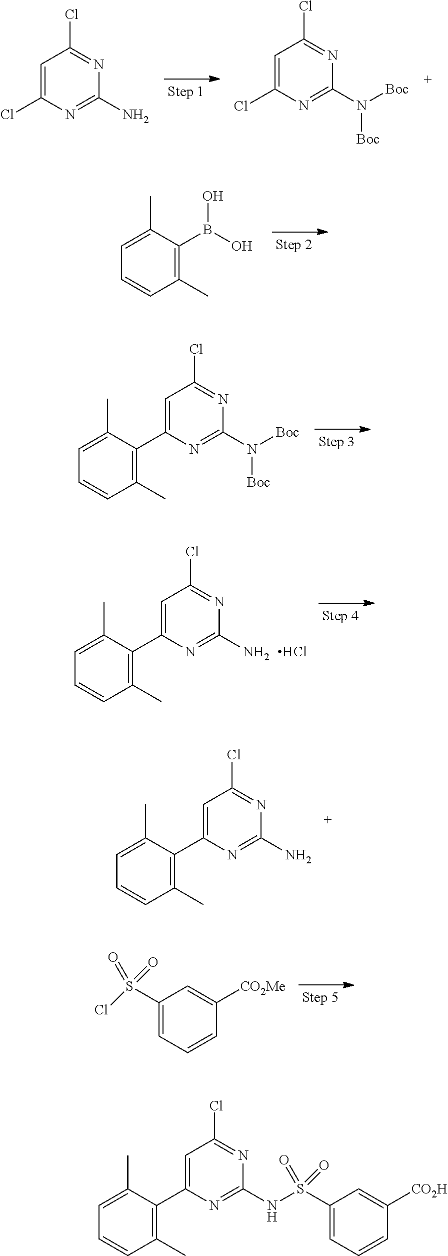

### Step 1: tert-Butyl N-tert-butoxycarbonyl-N-(4,6-dichloropyrimidin-2-yl)carbamate

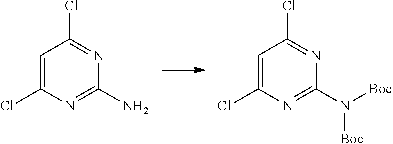 [SMILES: Nc1nc(Cl)cc(Cl)n1 ; CC(C)(C)OC(=O)N(C(=O)OC(C)(C)C)c1nc(Cl)cc(Cl)n1]

To a solution of 4,6-dichloropyrimidin-2-amine (300 g, 1.829 mol) in DCM (2.1 L) was added (BOC)₂O (838 g, 3.840 mol) followed by DMAP (5.6 g, 45.84 mmol). The mixture was stirred at ambient temperature for 6 h. Additional DMAP (5.6 g, 45.84 mmol) was added and the reaction was continued to stir at ambient temperature for 24 h. The mixture was diluted with water (2.1 L) and the organic phase separated. The organic phase was washed with water (2.1 L), 2.1 L of brine, dried over magnesium sulfate, filtered over Celite and concentrated in vacuo affording a light orange oil which had a silt in the slurry. The mixture was diluted with ˜500 mL of heptane and filtered using an M filter. The precipitate (SM) was washed with 250 mL of heptane. The filtrate was concentrated in vacuo affording a thick orange oil which was seeded with solid from a previous experiment and crystallized on standing, affording a light orange hard solid. tert-butyl N-tert-butoxycarbonyl-N-(4,6-dichloropyrimidin-2-yl)carbamate (645 g, 97%). ¹H NMR (400 MHz, DMSO-d₆) δ 8.07 (s, 1H), 1.44 (s, 18H). ESI-MS m/z calc. 363.07526, found 364.1 (M+1)+; Retention time: 2.12 minutes (LC method A).

### Step 2: tert-Butyl N-tert-butoxycarbonyl-N-[4-chloro-6-(2,6-dimethylphenyl)pyrimidin-2-yl]carbamate

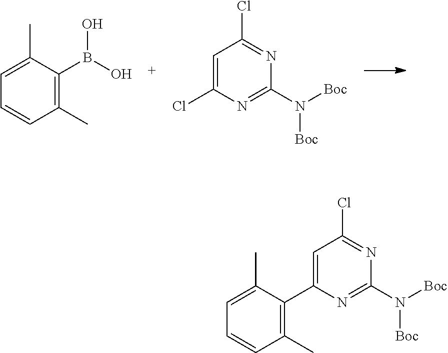 [SMILES: Cc1cccc(C)c1B(O)O ; CC(C)(C)OC(=O)N(C(=O)OC(C)(C)C)c1nc(Cl)cc(Cl)n1 ; Cc1cccc(C)c1-c1cc(Cl)nc(N(C(=O)OC(C)(C)C)C(=O)OC(C)(C)C)n1]

All solvents were degassed prior to use. To a slurry of tert-butyl N-tert-butoxycarbonyl-N-(4,6-dichloropyrimidin-2-yl)carbamate (88 g, 241.6 mmol), (2,6-dimethylphenyl)boronic acid (approximately 36.24 g, 241.6 mmol) and Cs₂CO₃ (approximately 196.8 g, 604.0 mmol) in DME (704 mL) and water (176 mL) were added. Pd(dppf)Cl₂ (approximately 8.839 g, 12.08 mmol) was added and the mixture was vigorously stirred under nitrogen at 80° C. (reflux) for 1 hour (no SM remained). The reaction was cooled to ambient temperature and diluted with water (704 mL). The aqueous phase was separated and extracted with EtOAc (704 mL). The organic phase was washed with 700 mL of brine, dried over magnesium sulfate, filtered and concentrated in vacuo. The crude product was chromatographed on a 1500 g silica gel column eluting with 0-30% EtOAc/hexanes. The product fractions (eluted at 15% EtOAc) were combined and concentrated in vacuo affording the product as a clear oil which crystallized on standing. tert-butyl N-tert-butoxycarbonyl-N-[4-chloro-6-(2,6-dimethylphenyl)pyrimidin-2-yl]carbamate (81.3 g, 78%). ¹H NMR (400 MHz, DMSO-d₆) δ 7.88 (s, 1H), 7.30 (dd, J=8.2, 7.0 Hz, 1H), 7.21-7.16 (m, 2H), 2.03 (s, 6H), 1.38 (s, 18H). ESI-MS m/z calc. 433.17682, found 434.1 (M+1)+; Retention time: 2.32 minutes (LC method A).

### Step 3: 4-Chloro-6-(2,6-dimethylphenyl)pyrimidin-2-amine (hydrochloride salt)

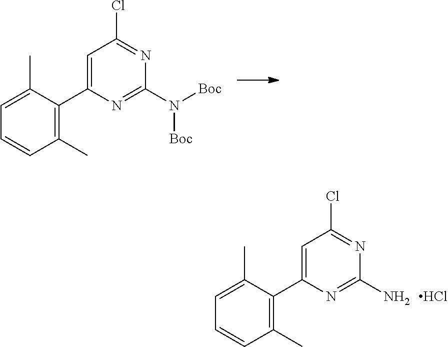 [SMILES: Cc1cccc(C)c1-c1cc(Cl)nc(N(C(=O)OC(C)(C)C)C(=O)OC(C)(C)C)n1 ; Cc1cccc(C)c1-c1cc(Cl)nc(N)n1 ; Cl]

tert-Butyl N-tert-butoxycarbonyl-N-[4-chloro-6-(2,6-dimethylphenyl) pyrimidin-2-yl]carbamate (514.8 g, 915.9 mmol) was dissolved in dichloromethane (4 L). Hydrogen chloride in p-dioxane (1 L, 4 mol) was added and the mixture was stirred overnight at room temperature. The resulting precipitate was collected by vacuum filtration and dried in vacuo to obtain 4-chloro-6-(2,6-dimethylphenyl)pyrimidin-2-amine hydrochloride as a white solid (213.5 g, 82%). ¹H NMR (250 MHz, DMSO-d₆) δ 7.45-6.91 (m, 3H), 6.73 (s, 1H), 2.08 (s, 6H). ESI-MS m/z calc. 233.072, found 234.1 (M+1)+; Retention time: 2.1 minutes (LC Method C).

### Step 4: 4-Chloro-6-(2,6-dimethylphenyl)pyrimidin-2-amine

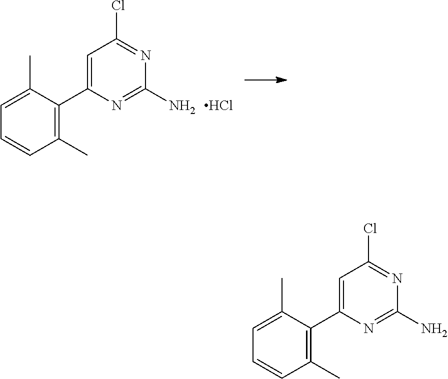 [SMILES: Cc1cccc(C)c1-c1cc(Cl)nc(N)n1 ; Cl ; Cc1cccc(C)c1-c1cc(Cl)nc(N)n1]

4-Chloro-6-(2,6-dimethylphenyl)pyrimidin-2-amine (hydrochloride salt) (166 g, 614.5 mmol) and 4-chloro-6-(2,6-dimethylphenyl)pyrimidin-2-amine (hydrochloride salt) (30 g, 111.0 mmol) were suspended in DCM (2.5 L), treated with NaOH (725 mL of 1 M, 725.0 mmol) and stirred at room temperature for 1 hour. The mixture was transferred into a separatory funnel and left standing over night. The DCM phase was separated and the aqueous phase with insoluble material was extracted twice more with DCM (2×500 mL). The combined brown DCM phases were stirred over magnesium sulfate and charcoal for 1 hour, filtered and the yellow solution concentrated to a volume of ˜ 500 mL. The solution was diluted with heptane (750 mL) and DCM was removed under reduced pressure at 60° C. to give a cream suspension. It was stirred at room temperature for 1 hour, filtered, washed with cold heptane and dried to give 4-chloro-6-(2,6-dimethylphenyl)pyrimidin-2-amine (157 g, 91%) as a cream solid. ¹H NMR (400 MHz, DMSO-d₆) δ 7.28-7.14 (m, 3H), 7.10 (d, J=7.5 Hz, 2H), 6.63 (s, 1H), 2.06 (s, 6H). ESI-MS m/z calc. 233.07198, found 234.0 (M+1)⁺; Retention time: 1.45 minutes (LC method A).

### Step 5: 3-[[4-Chloro-6-(2,6-dimethylphenyl)pyrimidin-2-yl]sulfamoyl]benzoic acid

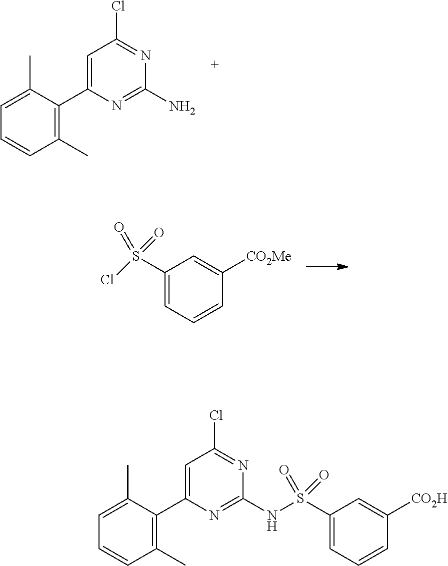 [SMILES: Cc1cccc(C)c1-c1cc(Cl)nc(N)n1 ; COC(=O)c1cccc(S(=O)(=O)Cl)c1 ; Cc1cccc(C)c1-c1cc(Cl)nc(NS(=O)(=O)c2cccc(C(=O)O)c2)n1]

4-Chloro-6-(2,6-dimethylphenyl)pyrimidin-2-amine (235 g, 985.5 mmol) was dissolved in MeTHF (2.3 L) and cooled in an ice bath under stirring and nitrogen. To the cold solution methyl 3-chlorosulfonylbenzoate (347 g, 1.479 mol) was added in one portion (seems slightly endothermic) and to the cold pale-yellow solution a solution of 2-methyl-butan-2-ol (Lithium salt) (875 mL of 3.1 M, 2.712 mol) (in heptane) was added dropwise over 1.25 hours (exothermic, internal temperature from 0 to 10° C.). The ice bath was removed and the greenish solution was stirred for 4 hours at room temperature. To the greenish solution cold HCl (2 L of 1.5 M, 3.000 mol) was added, the phases separated and the organic phase was washed once with water (1 L) and once with brine (500 mL). The aqueous phases were back extracted once with MeTHF (350 mL) and the organic phases were combined. This yellow MeTHF solution of methyl 3-[[4-chloro-6-(2,6-dimethylphenyl)pyrimidin-2-yl]sulfamoyl]benzoate (ESI-MS m/z calc. 431.07065, found 432.0 (M+1)+; Retention time: 1.81 minutes) was treated with NaOH (2.3 L of 2 M, 4.600 mol) and stirred at room temperature for 1 hour. The phases were separated and the NaOH phase was washed twice with MeTHF (2×500 mL) and the combined organic phases were extracted once with 2M NaOH (1×250 mL). The combined NaOH phases were combined, stirred in an ice bath and slowly acidified by addition of HCl (416 mL of 36% w/w, 4.929 mol) while keeping the internal temperature between 10 and 20° C. At the end of the addition (pH ˜5-6) the final pH was adjusted to 2-3 by addition of solid citric acid. The formed yellow tacky suspension was stirred at room temperature overnight to give a cream crisp suspension. The solid was collected by filtration, washed with plenty of water and sucked dry for 3 hours. The solid was dried under reduced pressure with a nitrogen leak at 45-50° C. for 120 hours 3-[[4-chloro-6-(2,6-dimethylphenyl)pyrimidin-2-yl]sulfamoyl]benzoic acid (395 g, 96%) was isolated as an off-white solid. ¹H NMR (400 MHz, DMSO-d₆) δ 13.44 (s, 1H), 12.46 (s, 1H), 8.48-8.39 (m, 1H), 8.25-8.15 (m, 1H), 8.15-8.08 (m, 1H), 7.68 (t, J=7.8 Hz, 1H), 7.31 (s, 1H), 7.28-7.18 (m, 1H), 7.10 (d, J=7.6 Hz, 2H), 1.84 (s, 6H). ESI-MS m/z calc. 417.055, found 418.0 (M+1)⁺; Retention time: 1.56 minutes. (LC method A).

### Example B: Preparation of N-[4-chloro-6-(2,6-dimethylphenyl)pyrimidin-2-yl]-3-nitro-benzenesulfonamide

### Step 1: N-[4-Chloro-6-(2,6-dimethylphenyl)pyrimidin-2-yl]-3-nitro-benzenesulfonamide

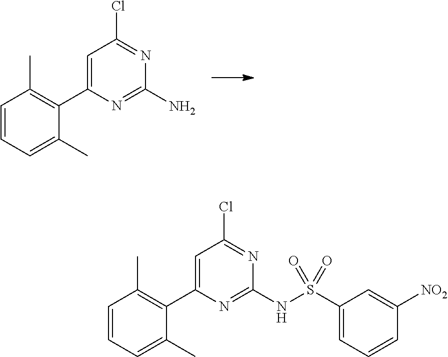 [SMILES: Cc1cccc(C)c1-c1cc(Cl)nc(N)n1 ; Cc1cccc(C)c1-c1cc(Cl)nc(NS(=O)(=O)c2cccc([N+](=O)[O-])c2)n1]

To a suspension of sodium hydride (60% in mineral oil) (4.87 g, 0.122 mol) in anhydrous tetrahydrofuran (30 mL) was added a solution of 4-chloro-6-(2,6-dimethylphenyl)pyrimidin-2-amine (8.13 g, 0.0348 mol) in anhydrous tetrahydrofuran (40 mL) dropwise at 0° C. The reaction mixture was stirred at room temperature for 30 minutes. A solution of 3-nitrobenzenesulfonyl chloride (11.57 g, 52.2 mmol) in anhydrous tetrahydrofuran (40 mL) was added to the reaction mixture dropwise at 0° C. The reaction was stirred at the same temperature for 1 hour. The reaction was quenched with a saturated aqueous solution of sodium bicarbonate (100 mL). The reaction solution was extracted with dichloromethane (3×100 mL). The combined organic layers were washed with water (100 mL), dried over anhydrous sodium sulfate, and then concentrated under vacuum. The residue was purified by silica gel column chromatography using 0 to 10% chloroform-ethyl acetate. The crude product was triturated with a solvent mixture of diethyl ether and hexane (1:5) to furnish N-[4-chloro-6-(2,6-dimethylphenyl)pyrimidin-2-yl]-3-nitro-benzenesulfonamide (5.98 g, 41%) as a white solid. ESI-MS m/z calc. 418.1, found 419.0 (M+1). Retention time: 5.73 minutes. ¹H NMR (250 MHz, CDCl₃) δ (ppm): 9.01 (s, 1H); 8.43 (t, J=10.5 Hz, 2H); 7.682 (t, J=7.8 Hz, 1H); 7.23 (m, 1H); 7.12 (d, J=7.5 Hz, 2H); 6.95 (s, 1H); 1.99 (s, 6H).

### Example C: Preparation of N-[4-(2,6-dimethylphenyl)-6-methylsulfonyl-pyrimidin-2-yl]-3-nitro-benzenesulfonamide

### Step 1: N-[4-(2,6-Dimethylphenyl)-6-methylsulfonyl-pyrimidin-2-yl]-3-nitro-benzenesulfonamide

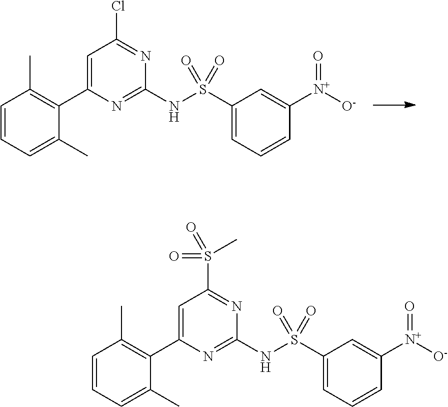 [SMILES: Cc1cccc(C)c1-c1cc(Cl)nc(NS(=O)(=O)c2cccc([N+](=O)[O-])c2)n1 ; Cc1cccc(C)c1-c1cc(S(C)(=O)=O)nc(NS(=O)(=O)c2cccc([N+](=O)[O-])c2)n1]

Stage 1: To a 250 mL round-bottomed flask were added N-[4-chloro-6-(2,6-dimethylphenyl)pyrimidin-2-yl]-3-nitro-benzenesulfonamide (14.14 g, 33.76 mmol), sodium thiomethoxide (5.86 g, 83.61 mmol) and NMP (130 mL). This solution was stirred at 100° C. for 3 h. The reaction mixture was then cooled to room temperature, quenched with 1 N HCl (300 mL), and extracted with ethyl acetate (3×300 mL). The combined organic extracts were washed with water (300 mL), 3% aqueous hydrogen peroxide solution (300 mL), water (300 mL) and saturated aqueous sodium chloride solution (300 mL), then dried over sodium sulfate, filtered, and evaporated in vacuo. This gave an orange foam (16.71 g, 115% crude product yield) that was carried onto the next reaction.

Stage 2: To a 250 mL round-bottomed flask containing the product from Stage 1, DCM (120 mL) was added, followed by m-CPBA (77% pure, 27.22 g, 121.5 mmol). This solution was stirred at room temperature for 90 min. The reaction mixture was quenched by transferring to a 1 L-Erlenmeyer flask containing DCM (400 mL) and solid Na₂S₂O₃ (41.15 g, 260.3 mmol). This mixture was stirred at room temperature for 1 h. The reaction mixture was diluted with DCM (300 mL), then washed with water (3×400 mL) and saturated aqueous sodium chloride solution (300 mL). The organic layer was then dried over sodium sulfate, filtered, and evaporated in vacuo. This solid was then partially dissolved in DCM (100 mL) and filtered in vacuo on a Buchner funnel to remove the m-chlorobenzoic acid waste (this was repeated three times). The remaining solution was then purified by silica gel chromatography (330 g of silica, 0 to 60% gradient of ethyl acetate/hexanes) to give N-[4-(2,6-dimethylphenyl)-6-methylsulfonyl-pyrimidin-2-yl]-3-nitro-benzenesulfonamide (5.881 g, 36%). ESI-MS m/z calc. 462.06677, found 463.1 (M+1)^{m}; Retention time: 1.6 minutes; LC method A.

### Example D: Preparation of dispiro[2.0.24.13]heptane-7-carbaldehyde

### Step 1: 1-Cyclopropylcyclopropanol

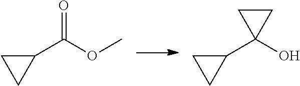

To a solution of methyl cyclopropanecarboxylate (75 g, 749.1 mmol) in ether (450 mL) was added titanium(IV) isopropoxide (55.3 mL, 187.4 mmol). To the mixture was slowly added ethyl magnesium bromide (1.6 L of 1 M, 1.60 mol) over 2 h. The addition is exothermic and controlled with monitoring the addition rate and using a cooling bath. The reaction temperature was kept between 21° C.-26° C. during addition. After addition, the mixture was stirred an additional 2 hours at ambient temperature. Next, the mixture was chilled to −5° C. using an acetone/dry ice bath and slowly quenched with sulfuric acid (970 g of 10% w/w, 990 mmol). The reaction mixture was cooled in a dry ice/acetone bath to keep the reaction vessel below 0° C. during the quench. As the quench progressed, a grey/purple solid formed. Following complete addition of aqueous sulfuric acid, the mixture was stirred at 0° C. for 1 h. The precipitate was filtered through Celite using a medium frit and the precipitate washed with diethyl ether (900 mL). The filtrate was transferred to a separatory funnel and the organic phase was washed with brine (1 L), saturated sodium bicarbonate (1 L) and brine (1 L). The organic phase was dried over magnesium sulfate, filtered over Celite and the solvent was evaporated by rotary evaporation at 100 torr and the water bath set at 20° C. The crude product was stored at −23° C. overnight and used without further purification. The product, 1-cyclopropylcyclopropanol (61 g, 83%) was found to contain ˜50% solvent (tetrahydrofuran and ⁱPrOH) and used as such in the next step. ¹H NMR (400 MHz, Chloroform-d) δ 1.32 (tt, J=8.2, 5.1 Hz, 1H), 0.71-0.61 (m, 2H), 0.51-0.43 (m, 2H), 0.43-0.33 (m, 2H), 0.23-0.14 (m, 2H).

### Step 2: 1-Bromo-1-cyclopropyl-cyclopropane

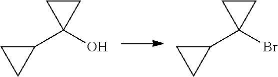 [SMILES: OC1(C2CC2)CC1 ; BrC1(C2CC2)CC1]

A solution of triphenylphosphine (56.1 g, 213.9 mmol) in dichloromethane (200 mL) was cooled to −10° C. A solution of bromine (11.0 mL, 214 mmol) in dichloromethane (40 mL) was added and the reaction was stirred at −10° C. for an additional 15 min. The reaction was then cooled to −30° C. and pyridine (3.3 mL, 41 mmol) was added. A solution of 1-cyclopropylcyclopropanol (20.0 g, 204 mmol), pyridine (17.3 mL, 214 mmol) and dichloromethane (100 mL) was added dropwise while maintaining the temperature between −15° C. to −20° C. After 30 min, the addition was complete and the reaction was allowed to gradually warm to room temperature. The reaction was then allowed to stir at 40° C. overnight. The reaction was then cooled to room temperature and quenched with water (100 mL). The reaction was then stirred for 10 min and the phases were separated. The organic phase was successively washed with 1 M hydrochloric acid (102 mL) then saturated sodium bicarbonate (50 mL), dried over sodium sulfate, filtered and concentrated (30° C./house vacuum ˜300 torr) to remove most of the dichloromethane. The crude reaction mixture was flash distilled (40° C./20 torr) to remove further dichloromethane. The solid residue (Ph₃PO and product) was re-heated and distilled (50-60° C./20 torr) to afford 21.5 g (65% yield) of 1-bromo-1-cyclopropyl-cyclopropane as a turbid, colorless liquid. ¹H NMR (400 MHz, Chloroform-d) δ 1.61 (tt, J=8.2, 5.0 Hz, 1H), 1.07-1.02 (m, 2H), 0.78-0.66 (m, 2H), 0.67-0.51 (m, 2H), 0.35-0.21 (m, 2H).

### Step 3: Cyclopropylidenecyclopropane

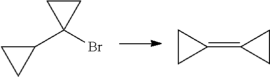 [SMILES: BrC1(C2CC2)CC1 ; C1CC1=C1CC1]

A solution of potassium tert-butoxide (16.7 g, 148.8 mmol) in dimethyl sulfoxide (100 mL) was stirred at room temperature in a 3-neck 250-mL round bottom flask. 1-Bromo-1-cyclopropyl-cyclopropane (20.0 g, 124.2 mmol) was added dropwise and the reaction immediately turned dark and then brown. The reaction was mildly exothermic (maintained temperature between 18° C. to 22° C. using an ice-water bath). After 10 min, the addition was completed. The ice-water bath was removed and the reaction was allowed to stir at room temperature. After 90 min, the reaction mixture was vacuum distilled using a bulb-to-bulb distillation. The distillation took place from 60° C. to 80° C. between 40 and 100 torr. The distillate slowly collected in the receiver to afford 18.2 g (7.3 g of product as a 42 wt % solution in t-BuOH) of a colorless liquid. The distillate was further washed with water (5×10 mL). Dichloromethane (4 g) was added and mixture was dried over magnesium sulfate, filtered (washing with 2 additional portions of 3 g of dichloromethane each) to afford 17.30 g (6.9 g product as a 39.6 wt % solution in dichloromethane; 69% yield) as a colorless liquid. ¹H NMR (400 MHz, Chloroform-d) δ 1.19 (s, 8H). The 1H NMR confirms the presence of dichloromethane and a small amount of tert-butanol.

### Step 4: Ethyl dispiro[2.0.2.1]heptane-7-carboxylate

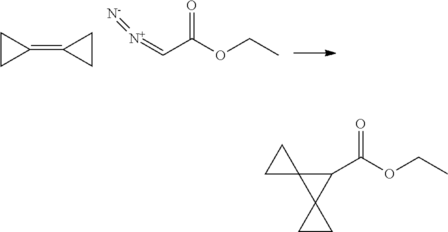

To a solution of cyclopropylidenecyclopropane (49.5 g, 617.8 mmol) in dichloromethane (110 mL) at 0° C. under a nitrogen atmosphere was added rhodium(II) acetate (4.2 g, 9.503 mmol). To the mixture at 0° C. was added ethyl 2-diazoacetate (106.8 mL, 1.016 mol) using a syringe pump set at an addition rate of 0.02 mL/min (1.2 mL/h). The addition was continuous for 89 hr. The crude reaction mixture was filtered through a plug of silica, washing 3× with 150 mL of dichloromethane each. The volatile materials were removed in vacuo affording a crude, dark yellow oil, ethyl dispiro[2.0.2.1]heptane-7-carboxylate (100 g, 97%, contains ˜20% dichloromethane, diethyl (E)-but-2-enedioate and diethyl (Z)-but-2-enedioate as contaminants) which was used directly in the next step. ¹H NMR (400 MHz, Chloroform-d) δ 4.13 (q, J=7.1 Hz, 2H), 2.23 (s, 1H), 1.24 (t, J=7.1 Hz, 3H), 1.08-0.93 (m, 4H), 0.90-0.82 (m, 2H), 0.77 (ddd, J=8.2, 5.0, 3.5 Hz, 2H).

### Step 5: Dispiro[2.0.2.1]heptan-7-yl methanol

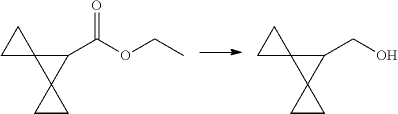

To a slurry of lithium aluminum hydride (7.8 g, 200.2 mmol) in diethyl ether (300 mL) chilled with an ice-water bath was slowly added ethyl dispiro[2.0.2.1]heptane-7-carboxylate (10.77 g, 64.79 mmol). The mixture was allowed to warm to a gentle reflux during the addition and continued to stir at ambient temperature for 1 h. The reaction was chilled with an ice-water bath and slowly quenched with the addition of water (8.0 mL, 440 mmol), followed by sodium hydroxide (8.0 mL of 2 M, 16 mmol) and then water (24.0 mL, 1.33 mol). The light yellow slurry was filtered over Celite and washed 3× with 150 mL of methyl tert-butyl ether. The filtrate was concentrated in vacuo affording 8.87 g of a clear oil, dispiro[2.0.2.1]heptan-7-yl methanol (8.87 g, quantitative yield). ¹H NMR (400 MHz, Chloroform-d) δ 3.71 (dd, J=6.7, 5.5 Hz, 2H), 1.76-1.65 (m, 1H), 1.46 (t, J=5.6 Hz, 1H), 0.87 (q, J=1.9 Hz, 4H), 0.72-0.61 (m, 2H), 0.60-0.50 (m, 2H).

### Step 6: Dispiro[2.0.24.13]heptane-7-carbaldehyde

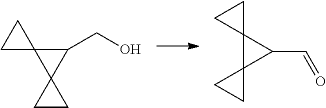 [SMILES: OCC1C2(CC2)C12CC2 ; O=CC1C2(CC2)C12CC2]

To a 20 mL vial was added {dispiro[2.0.2.1]heptan-7-yl}methanol (381 mg, 3.068 mmol), dichloromethane (4 mL), potassium bicarbonate (620 mg, 6.193 mmol), and pyridinium chlorochromate (728 mg, 3.377 mmol) (PCC). The reaction was allowed to stir at rt for 5 hours. The reaction was filtered over Celite and evaporated (300 torr, minimal heating in 40° C. water bath). The reaction mixture was dissolved in diethylether, filtered over Celite, and evaporated at 300 torr (minimal heating in 40° C. water bath) to provide dispiro[2.0.24.13]heptane-7-carbaldehyde (433 mg, 58%) as a pale brown oil. Purity estimated to be around 50%. The crude product was used in the next step without further purification.

### Example E: Preparation of 2-dispiro[2.0.24.13]heptan-7-ylacetaldehyde

### Step 1: 7-(Bromomethyl)dispiro[2.0.2.1]heptane

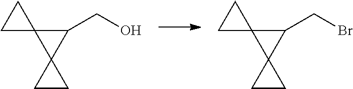 [SMILES: OCC1C2(CC2)C12CC2 ; BrCC1C2(CC2)C12CC2]

A 1000 mL, 3-neck round bottom flask was fitted with a mechanical stirrer, a cooling bath, an addition funnel, a J-Kem temperature probe and a nitrogen inlet/outlet. The vessel was charged under a nitrogen atmosphere with triphenylphosphine (102.7 mL, 443.2 mmol) and dichloromethane (1 L) which provided a clear colorless solution. Stirring was commenced and the cooling bath was charged with acetone. Dry ice was added in portions to the cooling bath until a pot temperature of −15° C. was obtained. The addition funnel was charged with a solution of bromine (22.82 mL, 443.0 mmol) in dichloromethane (220 mL, 10 mL/g) which was subsequently added dropwise over 1 h. Dry ice was added in portions to the cooling bath during the addition to maintain the pot temperature at −15° C. After the addition of bromine was completed, the pale yellow suspension was continued to stir at −15° C. for 15 min at which point the suspension was cooled to −30° C. The addition funnel was charged with a solution of dispiro[2.0.2.1]heptan-7-yl methanol (50 g, 402.6 mmol), pyridine (35.82 mL, 442.9 mmol) and dichloromethane (250 mL, 5 mL/g). The clear pale yellow solution was then added dropwise over 1.5 hours maintaining the pot temperature at −30° C. The resulting clear light yellow reaction mixture was allowed to gradually warm to a pot temperature of −5° C. and then continued to stir at −5° C. for 1 h. The reaction mixture then was poured into hexane (2000 mL) which resulted in the formation of a precipitate. The suspension was stirred at room temperature for 30 min and then filtered through a glass frit Buchner funnel with a 20 mm layer of celite. The clear filtrate was concentrated under reduced pressure (water bath temperature at 20° C.) to provide a yellow oil with some precipitate present. The oil was diluted with some hexane, allowed to stand at room temperature for 15 min and then filtered through a glass frit Buchner funnel with a 20 mm layer of celite. The clear filtrate was concentrated under reduced pressure (water bath temperature at 20° C.) to provide 7-(bromomethyl)dispiro[2.0.2.1]heptane (70 g, 93%) as a clear yellow oil. ¹H NMR (400 MHz, Chloroform-d) δ 3.49 (d, J=7.5 Hz, 2H), 1.90 (t, J=7.5 Hz, 1H), 1.06-0.84 (m, 4H), 0.71 (ddd, J=9.1, 5.1, 4.0 Hz, 2H), 0.54 (dddd, J=8.6, 4.8, 3.8, 1.0 Hz, 2H).

### Step 2: 2-Dispiro[2.0.2.1]heptan-7-ylacetonitrile

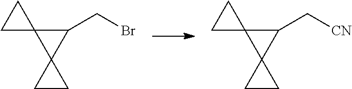 [SMILES: BrCC1C2(CC2)C12CC2 ; N#CCC1C2(CC2)C12CC2]

A 1000 mL, 3-neck round bottom flask was fitted with a mechanical stirrer, a cooling bath used as secondary containment, a J-Kem temperature probe and a nitrogen inlet/outlet. The vessel was charged under a nitrogen atmosphere with 7-(bromomethyl)dispiro[2.0.2.1]heptane (35 g, 187.1 mmol) and dimethyl sulfoxide (245 mL) which provided a clear amber solution. Stirring was commenced and the pot temperature was recorded at 19° C. The vessel was then charged with sodium cyanide (11.46 g, 233.8 mmol) added as a solid in one portion which resulted in a dark solution and a gradual exotherm to 49° C. over 15 min. After a few min the pot temperature began to decrease and the mixture was continued to stir at room temperature overnight (about 15 h). The dark reaction mixture was quenched with ice cold saturated sodium carbonate solution (500 mL) and then transferred to a separatory funnel and partitioned with diethyl ether (500 mL). The organic was removed and the residual aqueous was extracted with diethyl ether (2×250 mL). The combined organics were washed with water (500 mL), dried over sodium sulfate (200 g) and then filtered through a glass frit Buchner funnel. The clear amber filtrate was concentrated under reduced pressure (water bath temperature 20° C.) to provide 2-dispiro[2.0.2.1]heptan-7-ylacetonitrile (21 g, 84%) as a clear dark amber oil. ¹H NMR (400 MHz, Chloroform-d) δ 2.42 (d, J=6.6 Hz, 2H), 1.69 (t, J=6.6 Hz, 1H), 1.02-0.88 (m, 4H), 0.79-0.70 (m, 2H), 0.66-0.55 (m, 2H).

### Step 3: 2-Dispiro[2.0.2.1]heptan-7-ylacetic acid

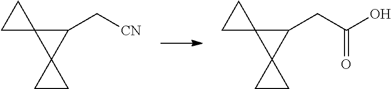 [SMILES: N#CCC1C2(CC2)C12CC2 ; O=C(O)CC1C2(CC2)C12CC2]

To a solution of 2-dispiro[2.0.2.1]heptan-7-ylacetonitrile (2.1 g, 14.19 mmol) in EtOH (32 mL) was added sodium hydroxide (5.12 g, 128.0 mmol) followed by water (13 mL) and the resulting solution was stirred and heated to 70° C. overnight. The mixture was then cooled to room temperature, diluted with water and extracted with diethyl ether. The aqueous phase was adjusted to pH=1 by the addition of 6 N hydrochloric acid (resulting in a cloudy precipitate) and extracted with diethyl ether (3×). The organic phases were dried (magnesium sulfate), filtered and concentrated giving 2-dispiro[2.0.2.1]heptan-7-ylacetic acid (2.19 g, 99% yield, 98% purity) as an orange solid which was used in the next step without further purification. ¹H NMR (400 MHz, Chloroform-d) δ 2.44 (d, J=6.9 Hz, 2H), 1.67 (t, J=6.9 Hz, 1H), 0.91 (ddd, J=9.0, 5.2, 3.9 Hz, 2H), 0.81 (dddd, J=8.9, 5.2, 3.9, 0.5 Hz, 2H), 0.69 (ddd, J=8.9, 5.2, 3.9 Hz, 2H), 0.56-0.44 (m, 2H).

### Step 4: 2-Dispiro[2.0.2.1]heptan-7-ylethanol

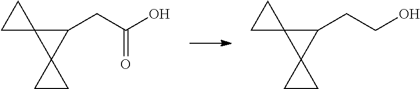 [SMILES: O=C(O)CC1C2(CC2)C12CC2 ; OCCC1C2(CC2)C12CC2]

To lithium aluminum hydride (827.4 mg, 902.3 μL, 21.80 mmol) dissolved in tetrahydrofuran (33.71 mL) cooled in an ice/water bath was added 2-dispiro[2.0.2.1]heptan-7-ylacetic acid (2.552 g, 16.77 mmol) in tetrahydrofuran (7.470 mL) dropwise over 15 min keeping the reaction temperature<20° C. The mixture was allowed to stir a total of 18 h, gradually warming to ambient temperature. The mixture was cooled with an ice/water bath and sequentially quenched with slow addition of water (838.4 mg, 838.4 μL, 46.54 mmol), followed by sodium hydroxide (1.006 mL of 5 M, 5.031 mmol), then water (2.493 g, 2.493 mL, 138.4 mmol) affording a white, granular slurry which was filtered over celite. Washed the filtered solid with diethyl ether. The filtrate was concentrated in vacuo at ˜300 mbar and 30° C. water bath. Diluted the residue with diethyl ether, dried (magnesium sulfate), filtered and concentrated in vacuo at ˜300 mbar and 30° C. water bath followed by ˜30 seconds under vacuum to give 2-dispiro[2.0.2.1]heptan-7-ylethanol (2.318 g, 100%) which was used directly in the ensuing step without further purification. ¹H NMR (400 MHz, Chloroform-d) δ 3.64 (s, 2H), 1.68 (d, J=6.7 Hz, 2H), 1.39 (s, 1H), 1.31 (s, 1H), 0.82 (d, J=14.0 Hz, 4H), 0.65 (s, 2H), 0.50 (d, J=3.6 Hz, 2H).

### Step 5: 2-Dispiro[2.0.24.13]heptan-7-ylacetaldehyde

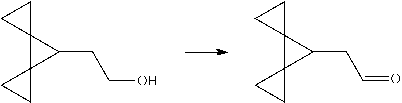 [SMILES: OCCC1C2(CC2)C12CC2 ; O=CCC1C2(CC2)C12CC2]

To a 20 mL vial was added 2-dispiro[2.0.24.13]heptan-7-ylethanol (506 mg of 65% w/w, 2.380 mmol), dichloromethane (3 mL), potassium bicarbonate (500 mg, 4.994 mmol), pyridinium chlorochromate (640 mg, 2.969 mmol) (PCC). The reaction was allowed to stir at rt for 5 hours. The reaction was filtered over Celite and evaporated. The reaction mixture was dissolved with ether, filtered over Celite, and evaporated at 300 torr (with minimal heating) to provide 2-dispiro[2.0.24.13]heptan-7-ylacetaldehyde (492 mg, 61%).

### Example F: Preparation of 3-[1-(trifluoromethyl)cyclopropyl]propan-1-ol

### Step 1: 2-[1-(Trifluoromethyl)cyclopropyl]ethyl methanesulfonate

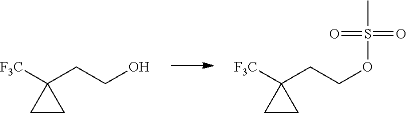

A 1000 mL, 3-neck round bottom flask was fitted with a mechanical stirrer, a cooling bath, a J-Kem temperature probe, an addition funnel and a nitrogen inlet/outlet. The vessel was charged under a nitrogen atmosphere with 2-[1-(trifluoromethyl)cyclopropyl]ethanol (125 g, 811.0 mmol) and 2-methyltetrahydrofuran (625 mL) which provided a clear colorless solution. Stirring was commenced and the pot temperature was recorded at 19° C. The vessel was then charged with triethylamine (124.3 mL, 891.8 mmol) added neat in one portion. The cooling bath was then charged with crushed ice/water and the pot temperature was lowered to 0° C. The addition funnel was charged with a solution of methanesulfonyl chloride (62.77 mL, 811.0 mmol) in 2-methyltetrahydrofuran (125 mL, 2 mL/g) which was subsequently added dropwise over 90 min which resulted in a white suspension and an exotherm to 1° C. The mixture was allowed to slowly warm to room temperature and continue to stir at room temperature for 1 hour at which point the mixture was poured into ice cold water (250 mL) and then transferred to a separatory funnel. The organic was removed and washed with 20 wt % potassium bicarbonate solution (250 mL), dried over sodium sulfate (200 g) and then filtered through a glass frit Buchner funnel. The clear filtrate was concentrated under reduced pressure to provide 2-[1-(trifluoromethyl)cyclopropyl]ethyl methanesulfonate (185 g, 98%) as a clear pale yellow oil. ¹H NMR (400 MHz, Chloroform-d) δ 4.36 (ddt, J=7.1, 6.4, 0.7 Hz, 2H), 3.02 (s, 3H), 2.03 (t, J=7.1 Hz, 2H), 1.11-0.98 (m, 2H), 0.81-0.66 (m, 2H).

### Step 2: 3-[1-(Trifluoromethyl)cyclopropyl]propanenitrile

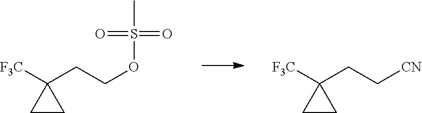

A 1000 mL, 3-neck round bottom flask was fitted with a mechanical stirrer, a heating mantle, a J-Kem temperature probe/controller, a water cooled reflux condenser and a nitrogen inlet/outlet. The vessel was charged under a nitrogen atmosphere with 2-[1-(trifluoromethyl)cyclopropyl]ethyl methanesulfonate (50 g, 215.3 mmol) and dimethyl sulfoxide (250 mL) which provided a clear pale yellow solution. Stirring was commenced and the pot temperature was recorded at 19° C. The vessel was charged with sodium cyanide (13.19 g, 269.1 mmol), added as a solid in one portion. The mixture was heated to a pot temperature of 70° C. and the condition was maintained for 24 h. Upon heating all of the sodium cyanide dissolved and the reaction mixture turned to a light amber suspension. After cooling to room temperature, the reaction mixture was poured into water (500 mL) and then transferred to a separatory funnel and partitioned with methyl tert-butyl ether (500 mL). The organic was removed and the residual aqueous was extracted with methyl tert-butyl ether (3×250 mL). The combined organic layers were washed with water (2×250 mL), dried over sodium sulfate (200 g) and then filtered through a glass frit Buchner funnel. The clear filtrate was concentrated under reduced pressure to provide 3-[1-(trifluoromethyl)cyclopropyl]propanenitrile (30 g, 85%) as a clear amber oil. ¹H NMR (400 MHz, Chloroform-d) δ 2.55 (t, J=7.6 Hz, 2H), 1.93 (t, J=7.7 Hz, 2H), 1.11-1.04 (m, 2H), 0.78-0.70 (m, 2H).

### Step 3: 3-[1-(Trifluoromethyl)cyclopropyl]propanoic acid

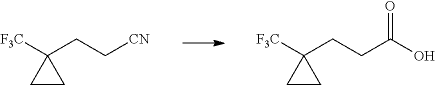 [SMILES: N#CCCC1(C(F)(F)F)CC1 ; O=C(O)CCC1(C(F)(F)F)CC1]

A 1000 mL, 3-neck round bottom flask was fitted with a mechanical stirrer, a heating mantle, a J-Kem temperature probe/controller, a water cooled reflux condenser and a nitrogen inlet/outlet. The vessel was subsequently charged under a nitrogen atmosphere with 3-[1-(trifluoromethyl)cyclopropyl]propanenitrile (25 g, 153.2 mmol) and ethyl alcohol (375 mL) which provided a clear amber solution. Stirring was commenced and the pot temperature was recorded at 19° C. The vessel was then charged with sodium hydroxide (102.1 mL of 6 M, 612.6 mmol), added in one portion. The resulting clear amber solution was heated to a pot temperature of 70° C. and the condition was maintained for 24 h. After cooling to room temperature, the reaction mixture was concentrated to remove the ethyl alcohol. The residual aqueous was diluted with water (150 mL) and then transferred to a separatory funnel and partitioned with methyl tert-butyl ether (50 mL). The aqueous was removed and the pH was adjusted to pH ˜1 with 6 M hydrochloric acid solution. The resulting aqueous solution was transferred to a separatory funnel and partitioned with methyl tert-butyl ether (250 mL). The organic was removed and the residual aqueous was extracted with methyl tert-butyl ether (2×150 mL). The combined organic was dried over sodium sulfate (150 g) and then filtered through a glass frit Buchner funnel. The clear filtrate was concentrated under reduced pressure to provide 3-[1-(trifluoromethyl)cyclopropyl]propanoic acid (26 g, 93%) as a clear amber oil. ¹H NMR (400 MHz, Chloroform-d) δ 2.63-2.50 (m, 2H), 1.96-1.84 (m, 2H), 1.03-0.95 (m, 2H), 0.66-0.58 (m, J 1.7 Hz, 2H).

### Step 4: 3-[1-(Trifluoromethyl)cyclopropyl]propan-1-ol

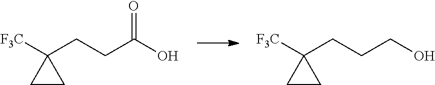 [SMILES: O=C(O)CCC1(C(F)(F)F)CC1 ; OCCCC1(C(F)(F)F)CC1]

A 1000 mL, 3-neck round bottom flask was fitted with a mechanical stirrer, a cooling bath, an addition funnel, a J-Kem temperature probe and a nitrogen inlet/outlet. The vessel was charged under a nitrogen atmosphere with lithium aluminum hydride pellets (6.775 g, 178.5 mmol). The vessel was then charged under a nitrogen atmosphere with tetrahydrofuran (250 mL). Stirring was commenced and the pot temperature was recorded at 20° C. The mixture was allowed to stir at room temperature for 0.5 hours to allow the pellets to dissolve. The pot temperature of the resulting grey suspension was recorded at 24° C. The cooling bath was then charged with crushed ice/water and the pot temperature was lowered to 0° C. The addition funnel was charged with a solution of 3-[1-(trifluoromethyl)cyclopropyl]propanoic acid (25 g, 137.3 mmol) in tetrahydrofuran (75 mL, 3 mL/g) and the clear pale yellow solution was added dropwise over 1 h. After the addition was completed, the pot temperature of the resulting greyish-brown suspension was recorded at 5° C. The mixture was allowed to slowly warm to room temperature and continue to stir at room temperature for 24 h. The suspension was cooled to 0° C. with a crushed ice/water cooling bath and then quenched by the very slow dropwise addition of water (6.775 mL), followed by 15 wt % sodium hydroxide solution (6.775 mL) and then finally with water (20.32 mL). The pot temperature of the resulting white suspension was recorded at 5° C. The suspension was continued to stir at ˜5° C. for 30 min and then filtered through a glass frit Buchner funnel with a 20 mm layer of celite. The filter cake was displacement washed with tetrahydrofuran (2×150 mL) and then dried under vacuum for 15 min. The filtrate was dried over sodium sulfate (250 g) and then filtered through a glass frit Buchner funnel. The filtrate was concentrated under reduced pressure to provide a clear pale amber oil as the desired product, 3-[1-(trifluoromethyl)cyclopropyl]propan-1-ol (21.2 g, 92%). ¹H NMR (400 MHz, Chloroform-d) δ 3.65 (t, J=6.0 Hz, 2H), 1.78-1.59 (m, 4H), 0.99-0.91 (m, 2H), 0.59 (dp, J=4.7, 1.7 Hz, 2H).

### Example G: Preparation of 2-[[4-chloro-6-(2,6-dimethylphenyl)pyrimidin-2-yl]sulfamoyl]pyridine-4-carboxylic acid

### Step 1: Methyl 2-[[4-chloro-6-(2,6-dimethylphenyl)pyrimidin-2-yl]sulfamoyl]pyridine-4-carboxylate

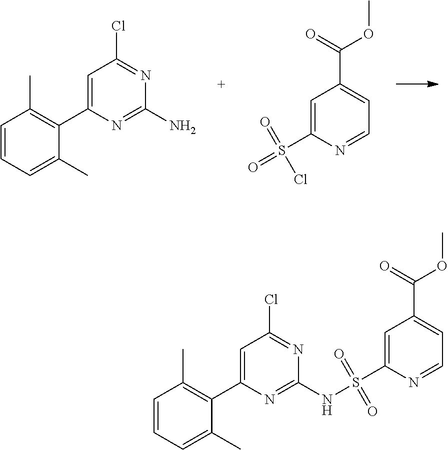 [SMILES: Cc1cccc(C)c1-c1cc(Cl)nc(N)n1 ; COC(=O)c1ccnc(S(=O)(=O)Cl)c1 ; COC(=O)c1ccnc(S(=O)(=O)Nc2nc(Cl)cc(-c3c(C)cccc3C)n2)c1]

Methyl 2-chlorosulfonylpyridine-4-carboxylate (5 g, 21.218 mmol) and 4-chloro-6-(2,6-dimethylphenyl)pyrimidin-2-amine (5 g, 21.395 mmol) were dissolved in anhydrous THE (150 mL) under nitrogen and the solution was cooled to −78 C. A 1M THE solution of LiHMDS (43 mL of 1 M, 43.000 mmol) was added dropwise and the mixture was allowed to warm up gradually to 0° C. The reaction mixture was quenched with saturated aqueous sodium bicarbonate (100 mL) and extracted with chloroform (3×50 mL). The organic fractions were combined, dried over sodium sulfate end evaporated. The residue was purified by silica gel column chromatography using 0-100% hexanes-ethyl acetate to give methyl 2-[[4-chloro-6-(2,6-dimethylphenyl)pyrimidin-2-yl]sulfamoyl]pyridine-4-carboxylate (8.3 g, 80.6%) as a white solid. ESI-MS m/z calc. 432.06592, found 432.8 (M+1)⁺; Retention time: 5.5 minutes; LC method S.

### Step 2: 2-[[4-Chloro-6-(2,6-dimethylphenyl)pyrimidin-2-yl]sulfamoyl]pyridine-4-carboxylic acid

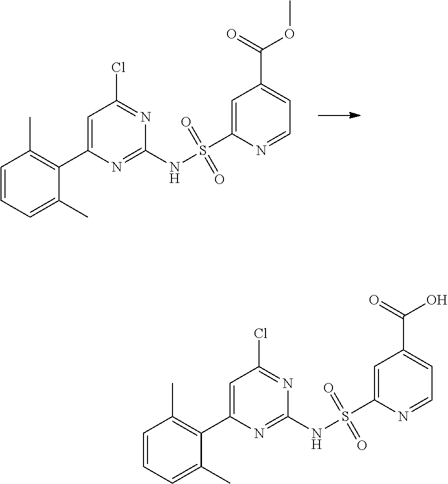 [SMILES: COC(=O)c1ccnc(S(=O)(=O)Nc2nc(Cl)cc(-c3c(C)cccc3C)n2)c1 ; Cc1cccc(C)c1-c1cc(Cl)nc(NS(=O)(=O)c2cc(C(=O)O)ccn2)n1]

A 1M aqueous NaOH solution (95 mL, 95.000 mmol) was added to a solution of methyl 2-[[4-chloro-6-(2,6-dimethylphenyl)pyrimidin-2-yl]sulfamoyl]pyridine-4-carboxylate (8.1 g, 18.712 mmol) in THE (95 mL) and the mixture was stirred at room temperature for 1 hour. 1M aqueous HCl solution was added to pH ˜8 and the mixture was extracted with 2-MeTHF (2×100 mL). The aqueous phase was separated and acidified with 1M aqueous HCl solution to pH-2. The formed precipitate was collected by filtration to give 2-[[4-chloro-6-(2,6-dimethylphenyl)pyrimidin-2-yl]sulfamoyl]pyridine-4-carboxylic acid (5.17 g, 71%) as a white solid. ¹H NMR (250 MHz, DMSO(d6)) δ 8.87 (d, J=5.0 Hz, 1H), 8.32 (d, J=1.1 Hz, 1H), 8.04 (dt, J=4.9, 1.5 Hz, 1H), 7.32-7.16 (m, 2H), 7.04 (d, J=7.5 Hz, 2H), 1.76 (s, 6H). ESI-MS m/z calc. 418.05026, found 419.3 (M+1)⁺; Retention time: 4.62 minutes; LC method S.

### V. Synthesis of New Compounds

### Example 1: Preparation of Compound 1 and Compound 2

### Step 1: N-(2-Aminoethyl)-2-nitro-benzenesulfonamide

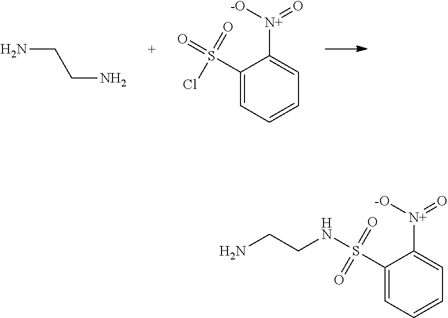

To a solution of ethane-1,2-diamine (600.00 mL, 8.89 mol) in tetrahydrofuran (500 mL) was added dropwise a solution of 2-nitrobenzenesulfonyl chloride (205 g, 897.26 mmol) in tetrahydrofuran (700 mL) at 0-5° C. under stirring, and under nitrogen atmosphere. After the addition, the reaction was stirred for an additional 30 minutes and then warmed to room temperature and concentrated in vacuo. The oily residue is taken up in DCM (500 mL) and washed with water (500 mL). The organic layer was separated, and the product was extracted from the aqueous layer (pH=11, 3×300 mL). The organic layers were combined and dried over anhydrous sodium sulfate, filtered, and concentrated in vacuo to give an orange gel (161 g, purity 90%). The aqueous layer was treated with 6 M HCl to adjust the pH to 8-7, then it was extracted with a mixture of chloroform:isopropanol (3:1, v:v, 4×300 mL). After evaporation, more material was isolated (57.2 g, purity 98%). The total amount of N-(2-aminoethyl)-2-nitro-benzenesulfonamide was 218.2 g (89%). ESI-MS m/z calc. 245.04703, found 246.4 (M+1)⁺; Retention time: 1.69 minutes; LC method T.

### Step 2: N-[2-[[(2R)-3-Chloro-2-hydroxy-propyl]amino]ethyl]-2-nitro-benzenesulfonamide

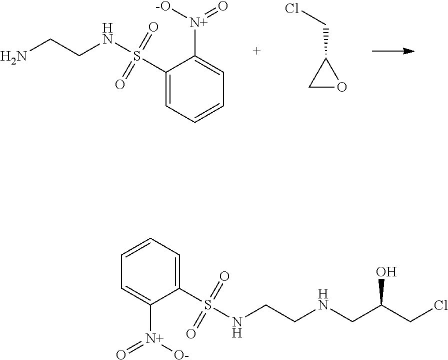

A 250 mL flask equipped with a magnetic stir bar was charged with N-(2-aminoethyl)-2-nitro-benzenesulfonamide (75 g, 275.22 mmol), anhydrous methanol (80 mL) and anhydrous magnesium sulfate (18 g, 149.54 mmol). Neat (2R)-2-(Chloromethyl)oxirane (12 mL, 147.35 mmol) was added quickly through syringe. The orange suspension was stirred under nitrogen at room temperature for 7 hours. The solid was filtered and the filtrate was concentrated under reduced pressure. The residue was taken in DCM (500 mL) and water (200 mL) and the two phases were separated. After drying over sodium sulfate, the solvent was evaporated. The residue was purified by flash chromatography on silica gel (2×330 g column) using a gradient of methanol (0 to 10%) in dichloromethane. The pure fractions were combined and the solvents removed under vacuum to give N-[2-[[(2R)-3-chloro-2-hydroxy-propyl]amino]ethyl]-2-nitro-benzenesulfonamide (40.2 g, 78%) as a yellow gel. ESI-MS m/z calc. 337.04993, found 338.4 (M+1)⁺; Retention time: 1.91 minutes; LC method T.

### Step 3: (6R)-1-(2-Nitrophenyl)sulfonyl-1,4-diazepan-6-ol

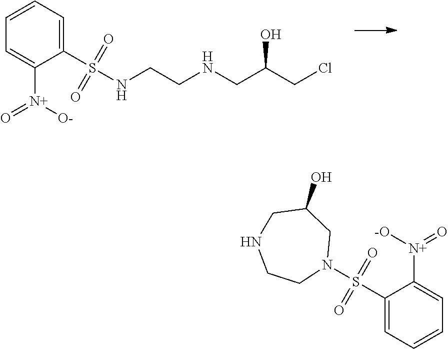

A suspension of N-[2-[[(2R)-3-chloro-2-hydroxy-propyl]amino]ethyl]-2-nitro-benzenesulfonamide (58.3 g, 167.42 mmol) and cesium carbonate (205 g, 629.19 mmol) in anhydrous acetonitrile (1500 mL) was stirred under nitrogen in an oil bath at 65° C. for 2.5 hours. After cooling down, the solids were filtered out and the solvent removed by evaporation. The residue was partitioned between DCM (800 mL) and water (200 mL) and the two phases were decanted. The organic phase was dried over sodium sulfate and the solvents evaporated. The crude residue was dissolved in DCM and purified by flash chromatography on silica gel (330 g column) using 0 to 15% methanol in dichloromethane. The pure fractions were combined and the solvents were evaporated to give (6R)-1-(2-nitrophenyl)sulfonyl-1,4-diazepan-6-ol (18.1 g, 34%) as an orange gel. ¹H NMR (250 MHz, DMSO) δ 8.06-7.93 (m, 2H), 7.93-7.77 (m, 2H), 5.01 (s, 1H), 3.85-3.65 (m, 2H), 3.62-3.50 (m, 2H), 3.25-3.09 (m, 3H), 2.96-2.64 (m, 4H). ESI-MS m/z calc. 301.07324, found 302.1 (M+1)⁺; Retention time: 0.88 minutes; LC method W.

### Step 4: tert-Butyl (6R)-6-hydroxy-4-(2-nitrophenyl)sulfonyl-1,4-diazepane-1-carboxylate

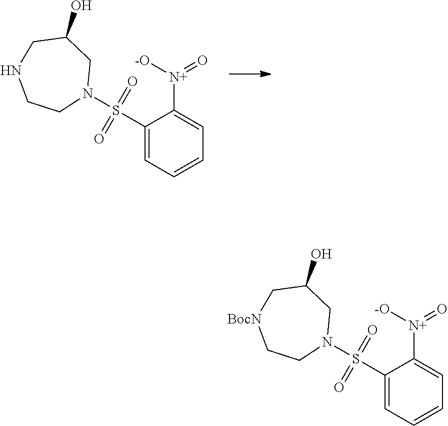

In a 500 mL flask, (6R)-1-(2-nitrophenyl)sulfonyl-1,4-diazepan-6-ol (12.14 g, 40.29 mmol) was dissolved under nitrogen in anhydrous methanol (130 mL). Triethylamine (8 mL, 57.40 mmol) was added and the mixture was cooled down in an ice bath. Di-tert-butyl dicarbonate (11 mL, 47.88 mmol) was added and the ice bath was removed after 5 min. The reaction mixture was stirred at room temperature for 20 hours. The reaction was concentrated, and the residue taken in DCM (100 mL) and saturated aqueous sodium bicarbonate (100 mL). The two phases were decanted, and the aqueous phase was further extracted with DCM (25 mL). The combined extracts were dried over sodium sulfate and the solvents evaporated to give a residue that was purified by flash chromatography on silica gel (330 g column) using a gradient of methanol (0 to 10% over 40 min) in dichloromethane. The product eluted around 2-3% methanol. The pure fractions were combined and the solvents evaporated to give tert-butyl (6R)-6-hydroxy-4-(2-nitrophenyl)sulfonyl-1,4-diazepane-1-carboxylate (13.93 g, 86%) as a yellow foamy solid. ¹H NMR (400 MHz, Chloroform-d) presence of several conformers visible δ 8.09-7.99 (m, 1H), 7.77-7.62 (m, 3H), 4.33-4.12 (m, 1H), 3.96-3.66 (m, 4H), 3.58-3.42 (m, 2H), 3.36-3.17 (m, 2H), 3.01-2.85 (m, 1H), 1.51-1.42 (m, 9H). ESI-MS m/z calc. 401.12567, found 402.28 (M+1)⁺; Retention time: 1.3 minutes; LC method A.

### Step 5: tert-Butyl (6S)-6-hydroxy-1,4-diazepane-1-carboxylate

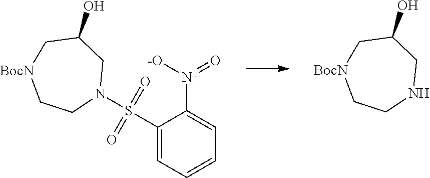

tert-Butyl (6R)-6-hydroxy-4-(2-nitrophenyl)sulfonyl-1,4-diazepane-1-carboxylate (52 g, 116.58 mmol) was dissolved in acetonitrile (500 mL) at rt. Potassium carbonate (97 g, 694.83 mmol) was added, followed by thiophenol (40.071 g, 38.5 mL, 352.79 mmol). The mixture was then heated in a 55° C. oil bath under nitrogen for 4 h. It was cooled to rt and concentrated to remove most acetonitrile. The residue was partitioned between DCM (500 mL) and HCl (400 mL, 1 N aqueous). The layers were separated, and the aqueous layer was washed two more times with DCM (200 mL×2). The aqueous solution (containing the desired product) was cooled in ice water. NaOH (3 N, aqueous) was added to reach pH=12. DCM was added to extract the free based product. The layers were again separated and the aqueous was further extracted (200 mL×2) with DCM. The combined DCM solutions were washed with brine (3200 mL), dried over anhydrous sodium sulfate, filtered and concentrated to afford tert-butyl (6S)-6-hydroxy-1,4-diazepane-1-carboxylate (15.6 g, 59%) ESI-MS m/z calc. 216.1474, found 217.4 (M+1)⁺; Retention time: 2.27 minutes. ESI-MS m/z calc. 216.1474, found 217.4 (M+1)⁺; Retention time: 2.27 minutes; LC method T.

### Step 6: tert-Butyl (6S)-4-[3-[[4-chloro-6-(2,6-dimethylphenyl)pyrimidin-2-yl]sulfamoyl]benzoyl]-6-hydroxy-1,4-diazepane-1-carboxylate

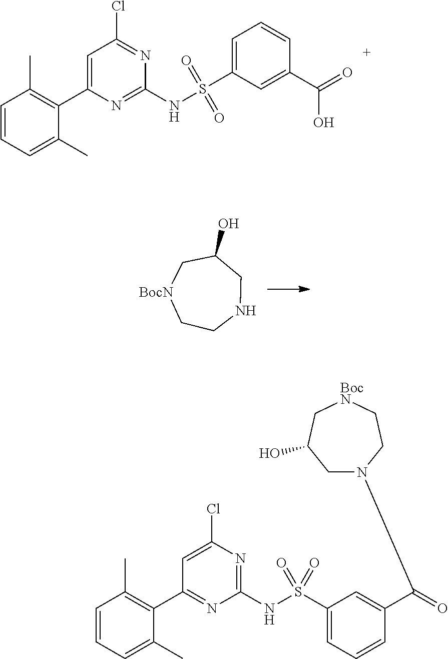

A 100 mL flask was charged under nitrogen with tert-butyl (6S)-6-hydroxy-1,4-diazepane-1-carboxylate (1.195 g, 5.525 mmol), anhydrous DMF (35 mL) and 3-[[4-chloro-6-(2,6-dimethylphenyl)pyrimidin-2-yl]sulfamoyl]benzoic acid (1.828 g, 4.375 mmol). After dissolution of the reagents, the mixture was cooled down in an ice bath. DIEA (5 mL, 28.71 mmol) and HATU (2.013 g, 5.294 mmol) were added and the mixture was stirred at 0° C. for 14 min then quenched by being poured in citric acid (150 mL of 10% w/v, 78.07 mmol)(10% aqueous) cooled in ice. The resulting white solid was filtered and washed with water. The wet solid was dissolved in DCM and the solution was dried over sodium sulfate. After evaporation of the solvents, the residue (2.76 g) was purified by flash chromatography on silica gel (120 g column) using a gradient of methanol (0 to 10% over 30 min) in dichloromethane. The product eluted around 4% methanol. Evaporation of the solvents gave tert-butyl (6S)-4-[3-[[4-chloro-6-(2,6-dimethylphenyl)pyrimidin-2-yl]sulfamoyl]benzoyl]-6-hydroxy-1,4-diazepane-1-carboxylate (1.672 g, 62%) as a white foamy solid. ¹H NMR (400 MHz, DMSO-d₆) complex spectra likely due to presence of rotamers, attribution of signals unclear, apparent excess of protons. δ 12.40 (s, 1H), 8.05-7.84 (m, 2H), 7.72-7.53 (m, 2H), 7.38-7.30 (m, 1H), 7.24 (t, J=7.3 Hz, 1H), 7.16-7.01 (m, 2H), 5.27 (d, J=13.5 Hz, 0.5H), 4.98 (s, 0.5H), 4.13-3.36 (m, 7H), 3.19 (broad s, 1H), 3.13-2.95 (m, 2H), 1.90 (d, J=9.2 Hz, 6H), 1.39 (d, J=17.4 Hz, 6H), 1.11 (s, 3H). ESI-MS m/z calc. 615.19183, found 616.41 (M+1)⁺; Retention time: 1.69 minutes; LC method A.

### Step 7: tert-Butyl (16S)-12-(2,6-dimethylphenyl)-2,8,8-trioxo-15-oxa-8λ⁶-thia-1,9,11,18,22-pentaazatetracyclo[14.4.1.13,7.110,14]tricosa-3,5,7(23),10,12,14(22)-hexaene-18-carboxylate

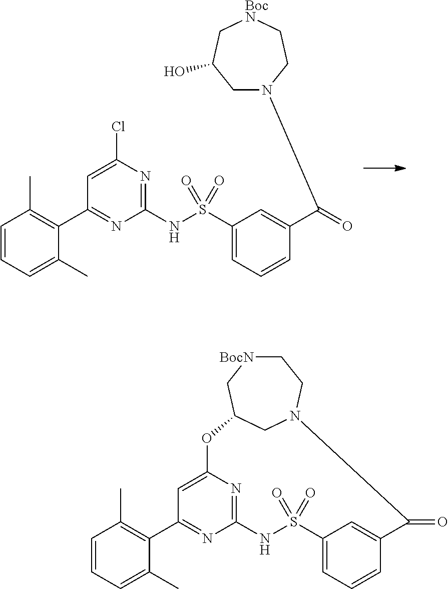 [SMILES: Cc1cccc(C)c1-c1cc(Cl)nc(NS(=O)(=O)c2cccc(C(=O)N3CCN(C(=O)OC(C)(C)C)C[C@@H](O)C3)c2)n1 ; Cc1cccc(C)c1-c1cc2nc(n1)NS(=O)(=O)c1cccc(c1)C(=O)N1CCN(C(=O)OC(C)(C)C)C[C@H](C1)O2]

A 250 mL flask was charged under nitrogen with tert-butyl (6S)-4-[3-[[4-chloro-6-(2,6-dimethylphenyl)pyrimidin-2-yl]sulfamoyl]benzoyl]-6-hydroxy-1,4-diazepane-1-carboxylate (1.426 g, 2.314 mmol) and anhydrous DMF (70 mL). The mixture was cooled down in ice. NaH (783 mg of 60% w/w, 19.58 mmol) (60% mineral oil dispersion) was added in two portions. The mixture was stirred under nitrogen at 0° C. for 3.5 hours. The mixture was slowly poured into an ice-cold citric acid (300 mL of 10% w/v, 156.1 mmol) (aqueous 10% solution) under stirring. The resulting solid suspension was extracted with EtOAc (4×60 mL). After drying over sodium sulfate, evaporation of the solvents gave a residue (3.97 g) that was purified by flash chromatography on silica gel (120 g column) using a gradient of methanol (0 to 10% over 30 min.) in dichloromethane. The product eluted around 4-5% methanol. Evaporation of the solvents gave tert-butyl (16S)-12-(2,6-dimethylphenyl)-2,8,8-trioxo-15-oxa-8λ⁶-thia-1,9,11,18,22-pentaazatetracyclo[14.4.1.13,7.110,14]tricosa-3,5,7(23),10,12,14(22)-hexaene-18-carboxylate (858 mg, 64%) as a colorless resin. ¹H NMR (400 MHz, DMSO-d₆) residual DMF visible. two conformers (55:45) observed δ 12.59 (two broad s, 1H), 8.33 (2 s, 1H), 7.95 (s, 2H overlapped with residual DMF signal), 7.67 (br s, 2H), 7.26 (t, J=7.6 Hz, 1H), 7.12 (d, J=7.6 Hz, 2H), 6.34 (br s, 1H), 5.42 (two br m, 1H), 4.61-4.38 (m, 1H), 4.23-3.84 (m, 2H), 3.64-3.36 (m, 2H), 3.32-3.09 (m, 2H), 2.04 (br s, 6H), 1.44 (two s, 9H). ESI-MS m/z calc. 579.21515, found 580.45 (M+1)⁺; Retention time: 1.51 minutes; LC method A.

### Step 8: (16R)-12-(2,6-Dimethylphenyl)-15-oxa-8λ⁶-thia-1,9,11,18,22-pentaazatetracyclo[14.4.1.13,7.110,14]tricosa-3,5,7(23),10,12,14(22)-hexaene-2,8,8-trione (Compound 2)

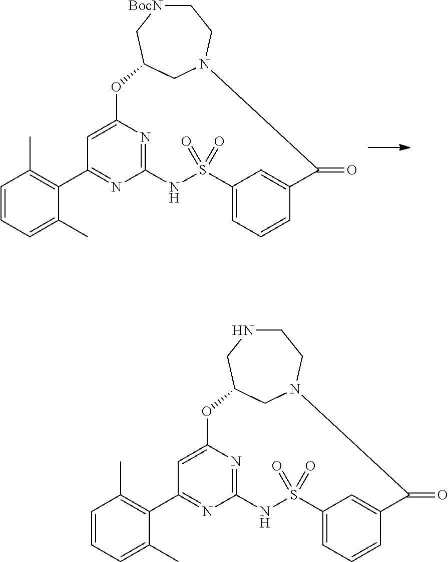 [SMILES: Cc1cccc(C)c1-c1cc2nc(n1)NS(=O)(=O)c1cccc(c1)C(=O)N1CCN(C(=O)OC(C)(C)C)C[C@H](C1)O2 ; Cc1cccc(C)c1-c1cc2nc(n1)NS(=O)(=O)c1cccc(c1)C(=O)N1CCNC[C@H](C1)O2]

tert-Butyl (16S)-12-(2,6-dimethylphenyl)-2,8,8-trioxo-15-oxa-8λ⁶-thia-1,9,11,18,22-pentaazatetracyclo[14.4.1.13,7.110,14]tricosa-3,5,7(23),10,12,14(22)-hexaene-18-carboxylate (21.68 g, 33.661 mmol) in DCM (200 mL, cooled in ice water bath) was treated with HCl (80 mL of 4 M, 320.00 mmol). The solution was then stirred at RT for 2 h. The mixture was concentrated to dryness. The residue was triturated with DCM/ether/hexanes (1/1/2, v:v, 40 mL). The supernatant was decanted. The residue was treated in this manner three times. The resulting solid was dried under high vacuum for 48 hours to give (16R)-12-(2,6-dimethylphenyl)-15-oxa-8λ⁶-thia-1,9,11,18,22-pentaazatetracyclo[14.4.1.13,7.110,14]tricosa-3,5,7(23),10,12,14(22)-hexaene-2,8,8-trione (hydrochloride salt) as a white solid (11.88 g, 66%). ¹H NMR (250 MHz, DMSO-d₆) δ 10.36 (s, 1H), 9.42 (s, 1H), 8.76 (s, 1H), 7.99-7.89 (m, 1H), 7.76-7.62 (m, 2H), 7.33-7.21 (m, 1H), 7.16-7.10 (m, 2H), 6.39 (d, J=0.9 Hz, 1H), 5.75 (m, 1H), 4.62-4.41 (m, 1H), 3.74-3.15 (m, 7H), 2.05 (s, 6H). ESI-MS m/z calc. 479.16272, found 480.1 (M+1)⁺; Retention time: 1.42 minutes; LC method W.

### Step 9: (16R)-18-(3,3-Dimethylbutyl)-12-(2,6-dimethylphenyl)-15-oxa-8λ⁶-thia-1,9,11,18,22-pentaazatetracyclo[14.4.1.13,7.110,14]tricosa-3,5,7(23),10,12,14(22)-hexaene-2,8,8-trione (Compound 1)

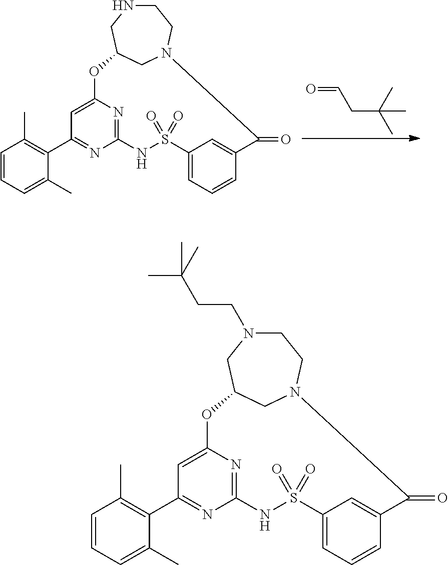

(16R)-12-(2,6-Dimethylphenyl)-15-oxa-8λ⁶-thia-1,9,11,18,22-pentaazatetracyclo[14.4.1.13,7.110,14]tricosa-3,5,7(23),10,12,14(22)-hexaene-2,8,8-trione (hydrochloride salt) (4.7 g, 9.108 mmol) was added to DCM (47 mL) under stirring and the suspension was treated with 3,3-dimethylbutanal (4.6 mL, 36.65 mmol) followed by acetic acid (3.1 mL, 54.51 mmol) and the resulting fine gelatinous suspension was stirred at room temperature for 50 minutes. The suspension was cooled in an ice bath and sodium cyanoborohydride (3.4 g, 54.10 mmol) was slowly added over ˜30 s resulting in an exothermic reaction. The suspension was stirred in the ice bath for 15 minutes, then the ice bath was removed and the suspension stirred for another 15 minutes. The reaction mixture was added to a stirred saturated solution of ammonium chloride (250 mL) and extracted with ethyl acetate (250 mL). The organic phase was washed once with a saturated solution of ammonium chloride (200 mL) and once with brine (100 mL). The aqueous phases was back extracted once with ethyl acetate (200 mL) and the combined organic phases were dried, filtered and evaporated. The crude product was purified by reverse phase chromatography (435 g Cis, liquid load with DMSO, and a few drops of 6M HCl) with a linear gradient of 5% acetonitrile to 100% acetonitrile in water containing 5 mM HCl. Impure fractions were repurified by the same method. The pure materials were combined to give (16R)-18-(3,3-dimethylbutyl)-12-(2,6-dimethylphenyl)-15-oxa-8λ⁶-thia-1,9,11,18,22-pentaazatetracyclo[14.4.1.13,7.110,14]tricosa-3,5,7(23),10,12,14(22)-hexaene-2,8,8-trione (hydrochloride salt) (3.56 g, 64%) as an off-white solid. ¹H NMR (400 MHz, DMSO-d₆) δ 10.59 (s, 1H), 8.82 (s, 1H), 7.95 (t, J=4.6 Hz, 1H), 7.69 (d, J=4.7 Hz, 2H), 7.27 (t, J=7.6 Hz, 1H), 7.13 (d, J=7.6 Hz, 2H), 6.40 (s, 1H), 5.82 (tt, J=10.5, 4.5 Hz, 1H), 4.51 (dt, J=14.8, 5.4 Hz, 1H), 3.95-3.61 (m, 4H), 3.55-3.21 (m, 5H), 2.05 (s, 6H), 1.71 (dp, J=17.3, 5.9 Hz, 2H), 0.95 (s, 9H). ESI-MS m/z calc. 563.25665, found 564.0 (M+1)⁺; Retention time: 4.75 minutes; LC method A with a 1-99% over 13.5 min gradient of phase B.

### Example 2: Preparation of Compound 3

### Step 1: (16R)-12-(2,6-Dimethylphenyl)-18-{spiro[3.5]nonan-2-yl}-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 3)

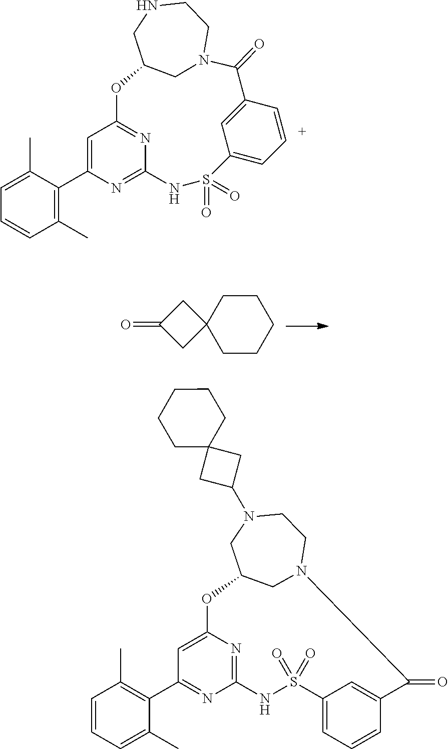

In a 20 mL vial, to a stirred solution of spiro[3.5]nonan-2-one (600 mg, 4.341 mmol) in anhydrous dichloromethane (40 mL) were added (16R)-12-(2,6-dimethylphenyl)-15-oxa-8λ⁶-thia-1,9,11,18,22-pentaazatetracyclo[14.4.1.13,7.110,14]tricosa-3,5,7(23),10,12,14(22)-hexaene-2,8,8-trione (hydrochloride salt) (2.0 g, 3.876 mmol), N,N-diisopropyl ethyl amine (1.1 mL, 6.315 mmol), and glacial acetic acid (450 μL, 7.913 mmol), in that order. The resulting light-yellow solution was stirred at ambient temperature for 25 min, then sodium triacetoxy borohydride (1.743 g, 8.224 mmol) was added at once and stirring continued for another hour. Then saturated aqueous sodium bicarbonate (5 mL) was added to the reaction and it was stirred for 20 min. The heterogeneous mixture was diluted with dichloromethane (10 mL), and the layers were separated. The aqueous layer was extracted with dichloromethane (2×10 mL). The combined organics were washed with brine (15 mL), dried over anhydrous sodium sulfate, filtered and concentrated under reduced pressure. The crude was purified by flash chromatography (80 g silica gel, 0-5% methanol in methylene chloride over 30 min). The desired product (16R)-12-(2,6-dimethylphenyl)-18-{spiro[3.5]nonan-2-yl}-15-oxa-8λ⁶-thia-1,9,11,18,22-pentaazatetracyclo[14.4.1.13,7.110,14]tricosa-3(23),4,6,10(22),11,13-hexaene-2,8,8-trione (1.649 g, 70%) was obtained as white solid. ¹H NMR (499 MHz, DMSO-d₆) δ 12.75 (s, 1H), 8.44 (s, 1H), 7.95-7.85 (m, 1H), 7.66 (d, J=5.6 Hz, 2H), 7.25 (t, J=7.6 Hz, 1H), 7.12 (d, J=7.6 Hz, 2H), 6.27 (s, 1H), 5.43 (t, J=9.2 Hz, 1H), 4.14 (dt, J=13.9, 6.8 Hz, 1H), 3.56 (dd, J=14.5, 4.0 Hz, 1H), 3.27 (dd, J=14.4, 10.8 Hz, 1H), 3.21-3.08 (m, 3H), 2.94 (dd, J=13.4, 7.0 Hz, 1H), 2.73-2.60 (m, 2H), 2.05 (s, 6H), 1.97 (t, J=9.3 Hz, 2H), 1.52 (t, J=9.6 Hz, 2H), 1.44 (d, J=6.0 Hz, 2H), 1.40 (d, J=5.5 Hz, 4H), 1.32 (s, 4H). ESI-MS m/z calc. 601.2723, found 602.5 (M+1)⁺; Retention time: 1.29 minutes; LC method A.

### Example 3: Preparation of Compound 4

### Step 1: (16R)-18-(4,4-Difluorocyclohexyl)-12-(2,6-dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 4)

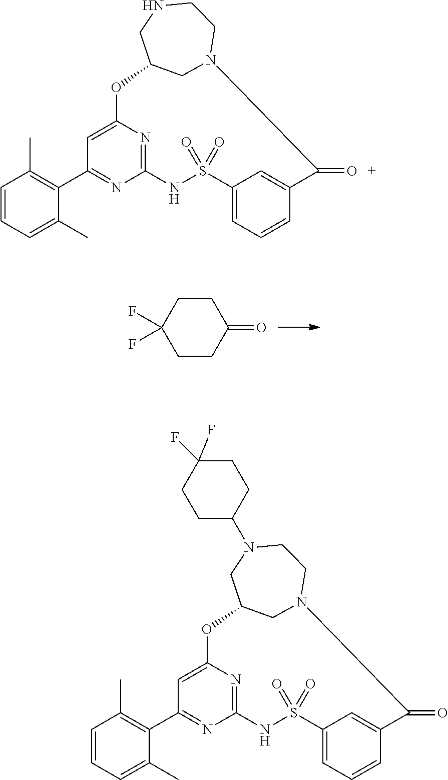

To a vial was added (16R)-12-(2,6-dimethylphenyl)-15-oxa-8λ⁶-thia-1,9,11,18,22-pentaazatetracyclo[14.4.1.13,7.110,14]tricosa-3,5,7(23),10,12,14(22)-hexaene-2,8,8-trione (hydrochloride salt) (65 mg, 0.1260 mmol), 4,4-difluorocyclohexanone (102 mg, 0.7605 mmol), 5-ethyl-2-methylpyridine borane complex (75 μL, 0.5038 mmol), and acetic acid (250 μL, 4.396 mmol). The reaction was gently heated at 35° C. for 4 hours. The reaction was quenched with methanol, filtered, and purified by reverse phase HPLC (1%-60% ACN:water with a 0.1% HCl modifier) to give (16R)-18-(4,4-difluorocyclohexyl)-12-(2,6-dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (40.1 mg, 53%) as a solid. ¹H NMR (400 MHz, DMSO-d₆) δ 8.71 (t, J=1.2 Hz, 1H), 8.01-7.92 (m, 1H), 7.78-7.68 (m, 2H), 7.31 (t, J=7.6 Hz, 1H), 7.16 (d, J=7.6 Hz, 2H), 6.30 (s, 1H), 5.80 (d, J=7.1 Hz, 1H), 4.48 (dt, J=15.0, 5.8 Hz, 1H), 3.91-3.77 (m, 3H), 3.65 (ddt, J=19.5, 13.7, 6.8 Hz, 3H), 3.58-3.43 (m, 1H), 3.30 (dd, J=14.5, 10.9 Hz, 1H), 2.31-2.14 (m, 4H), 2.06 (d, J=7.5 Hz, 6H), 1.98-1.79 (m, 3H). ESI-MS m/z calc. 597.2221, found 598.5 (M+1)⁺; Retention time: 1.13 minutes (LC method A).

### Example 4: Preparation of Compound 5

### Step 1: (16R)-18-(4,4-Dimethylcyclohexyl)-12-(2,6-dimethylphenyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione (Compound 5)

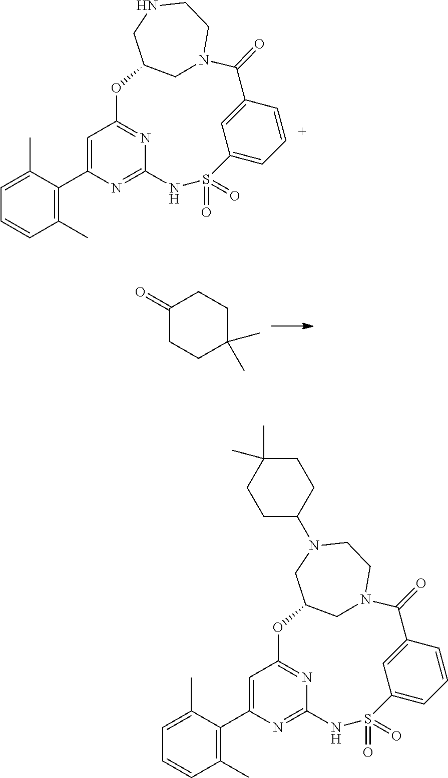

To a solution of (16R)-12-(2,6-dimethylphenyl)-15-oxa-8λ⁶-thia-1,9,11,18,22-pentaazatetracyclo[14.4.1.13,7.110,14]tricosa-3,5,7(23),10,12,14(22)-hexaene-2,8,8-trione (hydrochloride salt) (10 mg, 0.01938 mmol) in anhydrous dichloromethane (0.50 mL) were added 4,4-dimethylcyclohexanone (5 mg, 0.03962 mmol), triethylamine (5 μL, 0.03587 mmol), and glacial acetic acid (2 mg, 0.03330 mmol), in that order, at ambient temperature under nitrogen. The light-yellow solution was stirred for 20 min, then solid sodium triacetoxyborohydride (9 mg, 0.04246 mmol) was added at ambient temperature. After stirring for 12 hours (overnight), aqueous sodium bicarbonate (1 mL) and dichloromethane (2 mL) were added. The layers are separated, and the aqueous layer was extracted with dichloromethane (2×2 mL). The combined organic extracts were washed successively with water (2 mL) and brine (2 mL), dried over anhydrous sodium sulfate, filtered, and concentrated under reduced pressure. The crude material was taken up in DMSO (0.8 mL), microfiltered, and purified by preparative reverse-phase HPLC eluting with 0-99% acetonitrile in water over 15 min (HCl as a modifier). The desired product (16R)-18-(4,4-dimethylcyclohexyl)-12-(2,6-dimethylphenyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione (hydrochloride salt) (8 mg, 65%) obtained as a white solid. H NMR (400 MHz, DMSO-d₆) δ 10.27 (s, 1H), 8.78 (s, 1H), 7.94 (s, 1H), 7.69 (s, 2H), 7.27 (t, J=7.6 Hz, 1H), 7.13 (d, J=7.6 Hz, 2H), 6.36 (s, 1H), 5.84 (s, 1H), 4.50-4.32 (m, 1H), 3.99-3.83 (m, 2H), 3.80 (dd, J=14.7, 4.1 Hz, 1H), 3.66 (d, J=6.0 Hz, 2H), 3.56-3.43 (m, 2H), 3.30 (dd, J=14.3, 10.8 Hz, 1H), 2.05 (s, 6H), 1.89 (d, J=12.7 Hz, 2H), 1.74 (two t, J=12.2 Hz, 2H), 1.52 (d, J=13.1 Hz, 2H), 1.31 (t, J=13.4 Hz, 2H), 0.96 (s, 3H), 0.93 (s, 3H). ESI-MS m/z calc. 589.2723, found 590.5 (M+1)⁺; Retention time: 1.2 minutes; LC method A.

### Example 5: Preparation of Compound 6

### Step 1: (16R)-18-cyclopentyl-12-(2,6-dimethylphenyl)-8,8-dioxo-15-oxa-8λ⁶-thia-1,9,11,18,22-pentazatetracyclo[14.4.1.13,7.110,14]tricosa-3,5,7(23),10(22),11,13-hexaen-2-one (Compound 6)

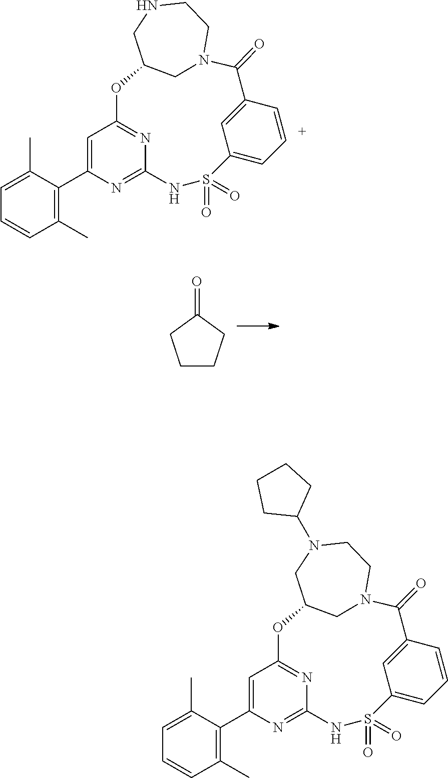 [SMILES: Cc1cccc(C)c1-c1cc2nc(n1)NS(=O)(=O)c1cccc(c1)C(=O)N1CCNC[C@H](C1)O2 ; O=C1CCCC1 ; Cc1cccc(C)c1-c1cc2nc(n1)NS(=O)(=O)c1cccc(c1)C(=O)N1CCN(C3CCCC3)C[C@H](C1)O2]

To a test tube was added (16R)-12-(2,6-dimethylphenyl)-15-oxa-8λ⁶-thia-1,9,11,18,22-pentaazatetracyclo[14.4.1.13,7.110,14]tricosa-3,5,7(23),10,12,14(22)-hexaene-2,8,8-trione (12 mg, 0.02502 mmol), DCE (0.5 mL), and DIEA (approximately 4.204 mg, 5.666 μL, 0.03253 mmol). After 5 minutes, cyclopentanone (approximately 10.52 mg, 11.06 μL, 0.1251 mmol) and acetic acid (approximately 7.513 mg, 7.115 μL, 0.1251 mmol) were added and the reaction was allowed to stir for 1 hour. Sodium cyanoborohydride (approximately 7.862 mg, 0.1251 mmol) was added and the reaction was allowed to stir at rt for 4 hours. The reactions were diluted with DMF (0.5 mL), filtered, and purified by reverse phase HPLC using a 1%-70% gradient of CAN in water with an HCl modifier. (16R)-18-cyclopentyl-12-(2,6-dimethylphenyl)-8,8-dioxo-15-oxa-8λ⁶-thia-1,9,11,18,22-pentazatetracyclo[14.4.1.13,7.110,14]tricosa-3,5,7(23),10(22),11,13-hexaen-2-one (hydrochloride salt) was isolated as a solid (5.3 mg, 36.3%). ESI-MS m/z calc. 547.22534, found 548.1 (M+1)⁺; Retention time: 0.94 minutes; LC method A.

### Example 6: Preparation of Compound 7

### Step 1: (16R)-18-(3-tert-Butylcyclobutyl)-12-(2,6-dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 7)

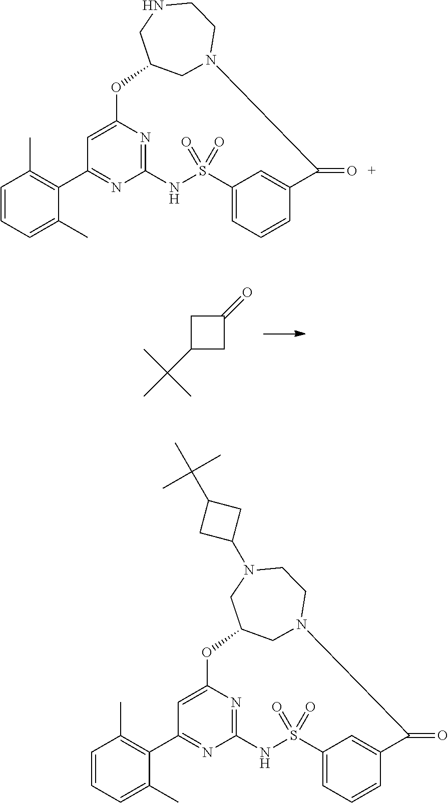

To a vial was added (16R)-12-(2,6-dimethylphenyl)-15-oxa-8λ⁶-thia-1,9,11,18,22-pentaazatetracyclo[14.4.1.13,7.110,14]tricosa-3,5,7(23),10,12,14(22)-hexaene-2,8,8-trione (hydrochloride salt) (100 mg, 0.1938 mmol), 3-tert-butylcyclobutanone (147 mg, 1.165 mmol), acetic acid (400 μL, 7.034 mmol), and 5-ethyl-2-methylpyridine borane complex (115 μL, 0.7725 mmol). The reaction was heated at 35° C. and allowed to stir overnight. The reaction was diluted with methanol, filtered, and purified by HPLC (1%-60% ACN:water with a 0.1% HCl modifier) to give (16R)-18-(3-tert-butylcyclobutyl)-12-(2,6-dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (90.1 mg, 74%) as a white solid. ESI-MS m/z calc. 589.2723, found 590.5 (M+1)⁺; Retention time: 1.27 minutes, LC method A.

### Example 7: Preparation of Compound 8

### Step 1: (16R)-12-(2,6-Dimethylphenyl)-18-{spiro[3.4]octan-2-yl}-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 8)

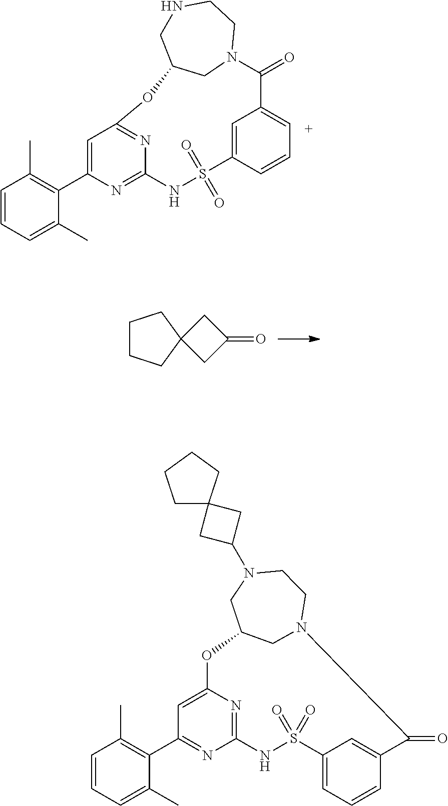 [SMILES: Cc1cccc(C)c1-c1cc2nc(n1)NS(=O)(=O)c1cccc(c1)C(=O)N1CCNC[C@H](C1)O2 ; O=C1CC2(CCCC2)C1 ; Cc1cccc(C)c1-c1cc2nc(n1)NS(=O)(=O)c1cccc(c1)C(=O)N1CCN(C3CC4(CCCC4)C3)C[C@H](C1)O2]

A 4 mL vial was charged with (16R)-12-(2,6-dimethylphenyl)-15-oxa-8λ⁶-thia-1,9,11,18,22-pentaazatetracyclo[14.4.1.13,7.110,14]tricosa-3,5,7(23),10,12,14(22)-hexaene-2,8,8-trione (hydrochloride salt) (55 mg, 0.1066 mmol), spiro[3.4]octan-2-one (73 mg, 0.5879 mmol), anhydrous DCM (1 mL), DIEA (28 μL, 0.1608 mmol) and acetic acid (40 μL, 0.7034 mmol). The vial was briefly purged with nitrogen, capped and stirred at room temperature for about 10 minutes. Sodium triacetoxyborohydride (66 mg, 0.3114 mmol) was added. The vial was purged with nitrogen, capped and the reaction was stirred at room temperature for 16 hours. Methanol (100 μL) was added. DCM was evaporated and the residue was taken in DMSO (1 mL). The solution was microfiltered through a PTFE syringe filter disc and purified by reverse phase preparative HPLC (Cis) using a gradient of acetonitrile in water (1 to 99% over 15 min) and HCl as a modifier. Evaporation gave a solid that was dissolved in DCM/MeOH for transfer into a vial. After evaporation of the solvents, trituration in DCM/hexanes and evaporation gave (16R)-12-(2,6-dimethylphenyl)-18-{spiro[3.4]octan-2-yl}-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (44 mg, 65%) as an off-white solid. ¹H NMR (500 MHz, DMSO-d₆) δ 11.37 (broad s, 1H), 8.83 (broad s, 1H), 7.95 (s, 1H), 7.69 (s, 2H), 7.27 (t, J=7.6 Hz, 1H), 7.13 (d, J=7.7 Hz, 2H), 6.35 (br s, 1H), 5.99-5.82 (m, 1H), 4.47-4.42 (m, 1H), 3.98 (h, J=8.5 Hz, 1H), 3.79 (dd, J=14.6, 4.0 Hz, 1H), 3.71-3.59 (m, 2H), 3.52-3.38 (m, likely 3H, overlapped with water signal) 2.40 (dq, J=26.8, 10.1, 8.9 Hz, 2H), 2.22 (q, J=8.4 Hz, 2H), 2.05 (br s, 6H), 1.72-1.45 (m, 10H). ESI-MS m/z calc. 587.25665, found 588.6 (M+1)⁺; Retention time: 1.16 minutes; LC method A.

### Example 8: Preparation of Compound 9 and Compound 10

### Step 1: (16R)-18-(2,2-Dimethylcyclobutyl)-12-(2,6-dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione, diastereomer 1 (Compound 9) and (16R)-18-(2,2-dimethylcyclobutyl)-12-(2,6-dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione, diastereomer 2 (Compound 10)

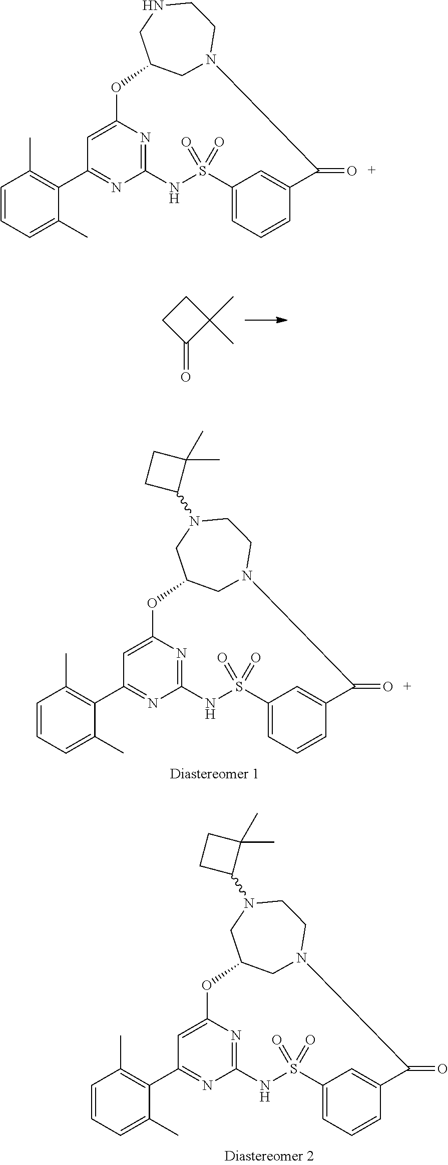

To a test tube was added (16R)-12-(2,6-dimethylphenyl)-15-oxa-8λ⁶-thia-1,9,11,18,22-pentaazatetracyclo[14.4.1.13,7.110,14]tricosa-3,5,7(23),10,12,14(22)-hexaene-2,8,8-trione (hydrochloride salt) (20 mg, 0.03876 mmol), 2,2-dimethylcyclobutanone (23 mg, 0.2344 mmol), 5-ethyl-2-methylpyridine borane complex (18 μL, 0.1209 mmol), and acetic acid (45 μL, 0.7913 mmol). The reaction was stirred at 30° C. overnight. The reaction was quenched with methanol, filtered, and purified by preparative HPLC (1%-50% MeCN over 30 minutes, HCl modifier). The first diastereomer to elute was (16R)-18-(2,2-dimethylcyclobutyl)-12-(2,6-dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (0.8 mg, 7%) ESI-MS m/z calc. 561.24097, found 562.3 (M+1)⁺; Retention time: 1.11 minutes (diastereomer 1). The second diastereomer to elute was (16R)-18-(2,2-dimethylcyclobutyl)-12-(2,6-dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (0.8 mg, 7%) ESI-MS m/z calc. 561.24097, found 562.5 (M+1)⁺; Retention time: 1.14 minutes (diastereomer 2), LC method A.

### Example 9: Preparation of Compound 11

### Step 1: (16R)-12-(2,6-Dimethylphenyl)-18-(1-ethylpropyl)-8,8-dioxo-15-oxa-8λ⁶-thia-1,9,11,18,22-pentazatetracyclo[14.4.1.13,7.110,14]tricosa-3,5,7(23),10,12,14(22)-hexaen-2-one (Compound 11)

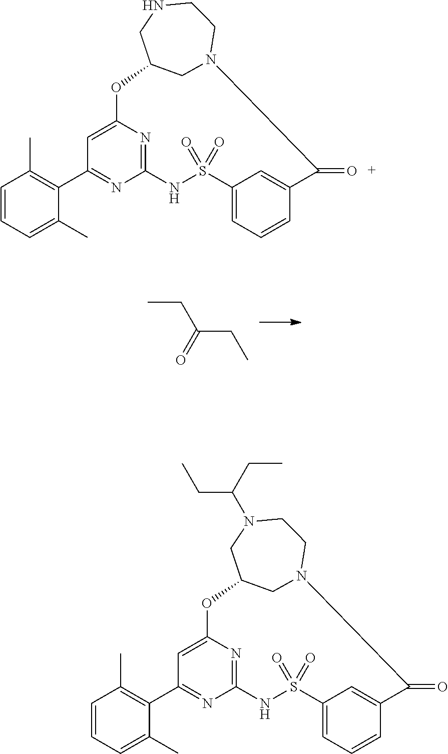

To a test tube was added (16R)-12-(2,6-dimethylphenyl)-15-oxa-8λ⁶-thia-1,9,11,18,22-pentaazatetracyclo[14.4.1.13,7.110,14]tricosa-3,5,7(23),10,12,14(22)-hexaene-2,8,8-trione (12 mg, 0.02502 mmol), DIEA (approximately 4.204 mg, 5.666 μL, 0.03253 mmol), and 1,2-dichloroethane (0.5 mL). After 5 minutes of stirring, pentan-3-one (approximately 10.78 mg, 13.26 μL, 0.1251 mmol), acetic acid (approximately 7.513 mg, 7.115 μL, 0.1251 mmol), and sodium triacetoxyborohydride (approximately 26.51 mg, 0.1251 mmol) were added and the reaction was allowed to stir overnight. The reaction was quenched with methanol, filtered, and purified by reverse phase HPLC to give (16R)-12-(2,6-dimethylphenyl)-18-(1-ethylpropyl)-8,8-dioxo-15-oxa-8λ⁶-thia-1,9,11,18,22-pentazatetracyclo[14.4.1.13,7.110,14]tricosa-3,5,7(23),10,12,14(22)-hexaen-2-one (hydrochloride salt) (14.9 mg, 100%). ESI-MS m/z calc. 549.24097, found 550.4 (M+1)⁺; Retention time: 1.12 minutes; LC method A.

### Example 10: Characterization of Compounds 12-34

The compounds in the following tables were prepared in a manner analogous to that described above using commercially available reagents and intermediates described herein.

TABLE 3

| Compound | | LCMS Rt | Calc. | | LCMS |
|---|---|---|---|---|---|
| number | Structure | (min) | mass | M + 1 | Method |
| 12 | 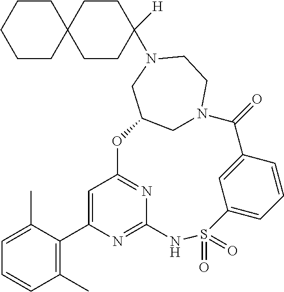 [SMILES: Cc1cccc(C)c1-c1cc2nc(n1)NS(=O)(=O)c1cccc(c1)C(=O)N1CCN(C3CCC4(CCCCC4)CC3)C[C@H](C1)O2] | 1.45 | 629.304 | 630.5 | A |
| 13 | 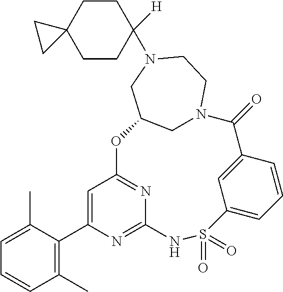 [SMILES: Cc1cccc(C)c1-c1cc2nc(n1)NS(=O)(=O)c1cccc(c1)C(=O)N1CCN(C3CCC4(CC3)CC4)C[C@H](C1)O2] | 1.18 | 587.257 | 588.4 | A |
| 14 | 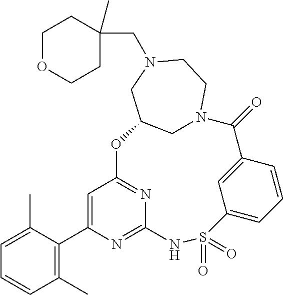 [SMILES: Cc1cccc(C)c1-c1cc2nc(n1)NS(=O)(=O)c1cccc(c1)C(=O)N1CCN(CC3(C)CCOCC3)C[C@H](C1)O2] | 1.27 | 591.252 | 592.3 | A |
| 15 | 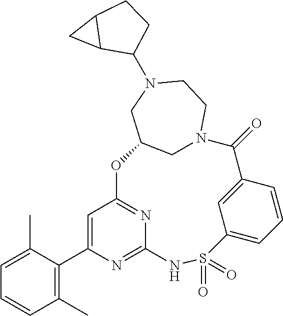 [SMILES: Cc1cccc(C)c1-c1cc2nc(n1)NS(=O)(=O)c1cccc(c1)C(=O)N1CCN(C3CCC4CC43)C[C@H](C1)O2] | 0.98 | 559.225 | 560.5 | A |
| 16 | 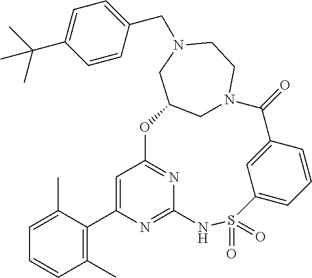 [SMILES: Cc1cccc(C)c1-c1cc2nc(n1)NS(=O)(=O)c1cccc(c1)C(=O)N1CCN(Cc3ccc(C(C)(C)C)cc3)C[C@H](C1)O2] | 1.43 | 625.272 | 626.5 | A |
| 17 | 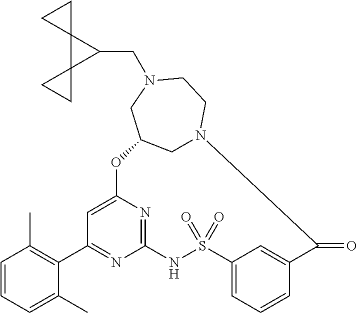 [SMILES: Cc1cccc(C)c1-c1cc2nc(n1)NS(=O)(=O)c1cccc(c1)C(=O)N1CCN(CC3C4(CC4)C34CC4)C[C@H](C1)O2] | 1.17 | 585.241 | 586.54 | A |
| 18 | 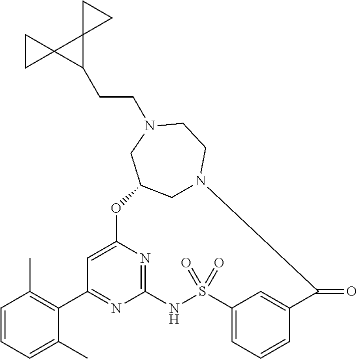 [SMILES: Cc1cccc(C)c1-c1cc2nc(n1)NS(=O)(=O)c1cccc(c1)C(=O)N1CCN(CCC3C4(CC4)C34CC4)C[C@H](C1)O2] | 1.26 | 599.257 | 600.58 | A |
| 19 | 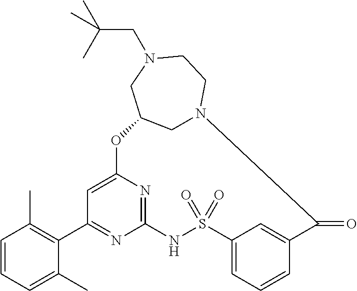 [SMILES: Cc1cccc(C)c1-c1cc2nc(n1)NS(=O)(=O)c1cccc(c1)C(=O)N1CCN(CC(C)(C)C)C[C@H](C1)O2] | 1.24 | 549.241 | 550.49 | A |
| 20 | 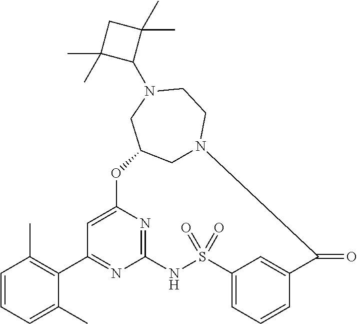 [SMILES: Cc1cccc(C)c1-c1cc2nc(n1)NS(=O)(=O)c1cccc(c1)C(=O)N1CCN(C3C(C)(C)CC3(C)C)C[C@H](C1)O2] | 1.28 | 589.272 | 590.2 | A |
| 21 | 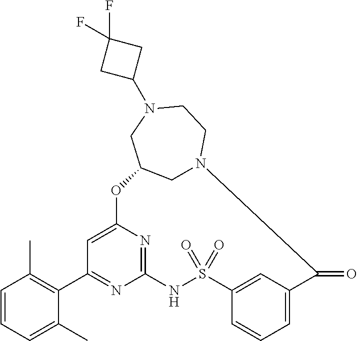 [SMILES: Cc1cccc(C)c1-c1cc2nc(n1)NS(=O)(=O)c1cccc(c1)C(=O)N1CCN(C3CC(F)(F)C3)C[C@H](C1)O2] | 1.32 | 569.191 | 570.5 | A |
| 22 | 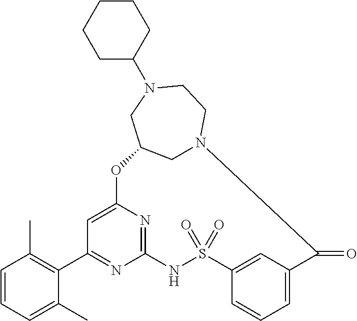 [SMILES: Cc1cccc(C)c1-c1cc2nc(n1)NS(=O)(=O)c1cccc(c1)C(=O)N1CCN(C3CCCCC3)C[C@H](C1)O2] | 1.1 | 561.241 | 562.4 | A |
| 23 | 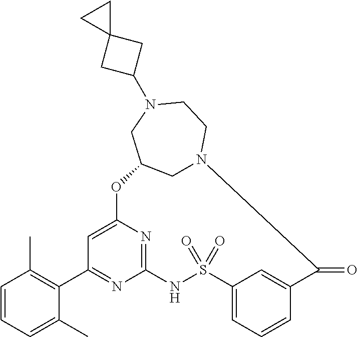 [SMILES: Cc1cccc(C)c1-c1cc2nc(n1)NS(=O)(=O)c1cccc(c1)C(=O)N1CCN(C3CC4(CC4)C3)C[C@H](C1)O2] | 1.03 | 559.225 | 560.2 | A |
| 24 | 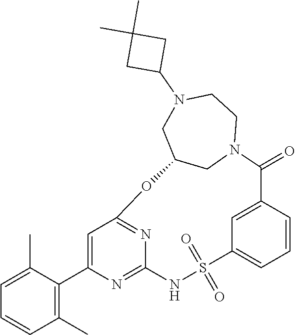 [SMILES: Cc1cccc(C)c1-c1cc2nc(n1)NS(=O)(=O)c1cccc(c1)C(=O)N1CCN(C3CC(C)(C)C3)C[C@H](C1)O2] | 1.05 | 561.241 | 562.1 | A |
| 25 | 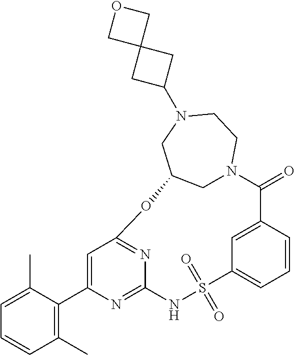 [SMILES: Cc1cccc(C)c1-c1cc2nc(n1)NS(=O)(=O)c1cccc(c1)C(=O)N1CCN(C3CC4(COC4)C3)C[C@H](C1)O2] | 0.85 | 575.22 | 576.2 | A |
| 26 | 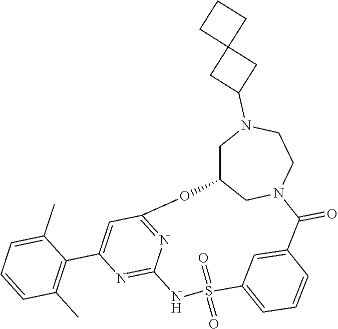 [SMILES: Cc1cccc(C)c1-c1cc2nc(n1)NS(=O)(=O)c1cccc(c1)C(=O)N1CCN(C3CC4(CCC4)C3)C[C@H](C1)O2] | 1.1 | 573.241 | 574.2 | A |
| 27 | 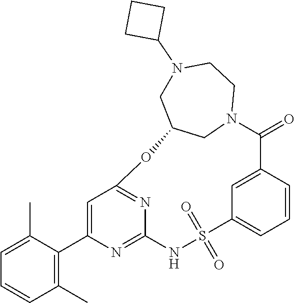 [SMILES: Cc1cccc(C)c1-c1cc2nc(n1)NS(=O)(=O)c1cccc(c1)C(=O)N1CCN(C3CCC3)C[C@H](C1)O2] | 0.88 | 533.21 | 534 | A |
| 28 | 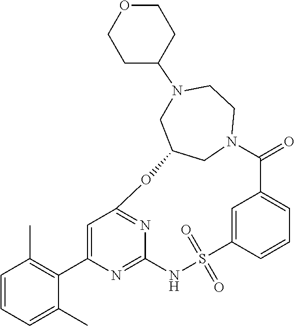 [SMILES: Cc1cccc(C)c1-c1cc2nc(n1)NS(=O)(=O)c1cccc(c1)C(=O)N1CCN(C3CCOCC3)C[C@H](C1)O2] | 0.85 | 563.22 | 564.2 | A |
| 29 | 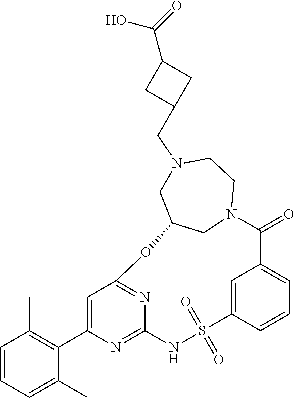 [SMILES: Cc1cccc(C)c1-c1cc2nc(n1)NS(=O)(=O)c1cccc(c1)C(=O)N1CCN(CC3CC(C(=O)O)C3)C[C@H](C1)O2] | 0.93 | 591.215 | 592.2 | A |
| 30 | 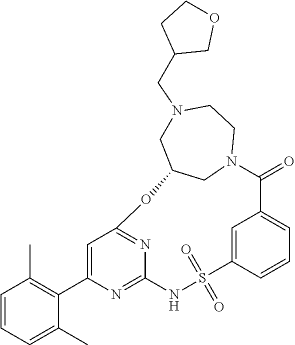 [SMILES: Cc1cccc(C)c1-c1cc2nc(n1)NS(=O)(=O)c1cccc(c1)C(=O)N1CCN(CC3CCOC3)C[C@H](C1)O2] | 0.89 | 563.22 | 564 | A |
| 31 | 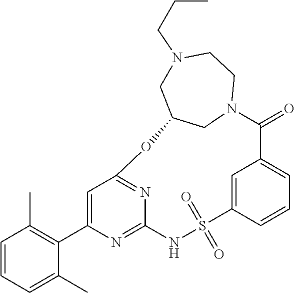 [SMILES: CCCN1CCN2C[C@@H](C1)Oc1cc(-c3c(C)cccc3C)nc(n1)NS(=O)(=O)c1cccc(c1)C2=O] | 0.88 | 521.21 | 522 | A |
| 32 | 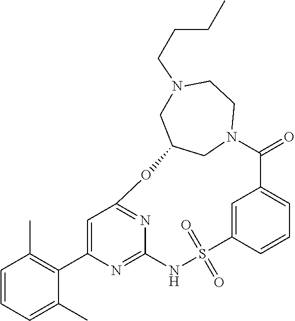 [SMILES: CCCCN1CCN2C[C@@H](C1)Oc1cc(-c3c(C)cccc3C)nc(n1)NS(=O)(=O)c1cccc(c1)C2=O] | 0.97 | 535.225 | 536 | A |
| 33 | 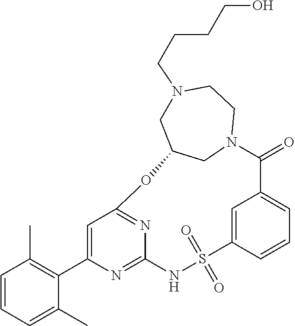 [SMILES: Cc1cccc(C)c1-c1cc2nc(n1)NS(=O)(=O)c1cccc(c1)C(=O)N1CCN(CCCCO)C[C@H](C1)O2] | 0.8 | 551.22 | 552.2 | A |
| 34 | 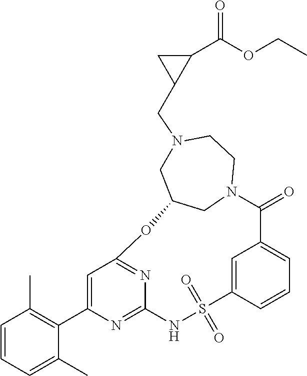 [SMILES: CCOC(=O)C1CC1CN1CCN2C[C@@H](C1)Oc1cc(-c3c(C)cccc3C)nc(n1)NS(=O)(=O)c1cccc(c1)C2=O] | 1.03 | 605.231 | 606.1 | A |

TABLE 4

| Com- | |
|---|---|
| pound | |
| number | NMR |
| 12 | ¹H NMR (499 MHz, Methanol-d₄) δ 8.87 (s, 1H), 8.06 (dt, J = 7.6, 1.5 Hz, |
| | 1H), 7.75 (dt, J = 7.7, 1.5 Hz, 1H), 7.71 (t, J = 7.7 Hz, 1H), 7.28 (t, J = 7.6 |
| | Hz, 1H), 7.14 (d, J = 7.6 Hz, 2H), 6.29 (s, 1H), 6.10-5.98 (m, 1H), 4.69- |
| | 4.57 (m, 1H), 4.01 (d, J = 13.8 Hz, 1H), 3.91 (d, J = 7.5 Hz, 2H), 3.83 (dt, |
| | J = 13.6, 6.6 Hz, 1H), 3.74-3.63 (m, 1H), 3.61-3.46 (m, 2H), 3.38-3.32 |
| | (m, 1H), 2.11 (s, 6H), 1.94 (s, 2H), 1.91-1.74 (m, 4H), 1.46 (s, 8H), 1.22 |
| | (s, 2H), 1.15 (t, J = 13.3 Hz, 1H), 0.98 (t, J = 13.3 Hz, 1H). |
| 13 | ¹H NMR (499 MHz, Methanol-d₄) δ 8.81 (s, 1H), 8.06 (dt, J = 7.7, 1.6 Hz, |
| | 1H), 7.76 (dt, J = 7.7, 1.5 Hz, 1H), 7.71 (t, J = 7.7 Hz, 1H), 7.28 (t, J = 7.7 |
| | Hz, 1H), 7.15 (d, J = 7.6 Hz, 2H), 6.30 (s, 1H), 5.98 (s, 1H), 4.71-4.57 (m, |
| | 1H), 4.07-4.00 (m, 1H), 4.00-3.90 (m, 2H), 3.90-3.80 (m, 1H), 3.79- |
| | 3.68 (m, 1H), 3.68-3.54 (m, 2H), 3.37-3.32 (m, 1H), 2.24-2.14 (m, 2H), |
| | 2.11 (s, 6H), 2.00-1.91 (m, 1H), 1.91-1.76 (m, 3H), 1.17-1.01 (m, 2H), |
| | 0.46-0.38 (m, 2H), 0.37-0.29 (m, 2H). |
| 14 | ¹H NMR (499 MHz, DMSO-d₆) δ 8.58 (s, 1H), 7.96-7.87 (m, 1H), 7.68 (d, |
| | J = 4.7 Hz, 2H), 7.26 (t, J = 7.6 Hz, 1H), 7.12 (d, J = 7.7 Hz, 2H), 6.27 (s, |
| | 1H), 5.80-5.62 (m, 1H), 4.37-4.25 (m, 1H), 3.66 (dd, J = 10.1, 5.6 Hz, |
| | 7H), 3.50-3.22 (m, 4H), 2.57-2.51 (m, 2H), 2.04 (s, 6H), 1.68-1.50 (m, |
| | 2H), 1.48-1.28 (m, 2H), 1.16 (s, 3H). |
| 15 | ¹H NMR (500 MHZ, Methanol-d₄) δ 8.72 (s, 1H), 8.07 (d, J = 7.6 Hz, 1H), |
| | 7.80-7.75 (m, 1H), 7.72 (t, J = 7.7 Hz, 1H), 7.28 (t, J = 7.7 Hz, 1H), 7.15 |
| | (d, J = 7.7 Hz, 2H), 6.30 (d, J = 7.5 Hz, 1H), 5.97-5.84 (m, 1H), 4.72- |
| | 4.59 (m, 1H), 4.32-4.25 (m, 1H), 4.24-4.10 (m, 1H), 4.09-4.04 (m, 1H), |
| | 3.97-3.84 (m, 2H), 3.83-3.65 (m, 1H), 3.65-3.54 (m, 1H), 3.36-3.32 |
| | (m, 1H), 2.97-2.68 (m, 1H), 2.29-2.17 (m, 1H), 2.11 (s, 6H), 2.00-1.94 |
| | (m, 1H), 1.84-1.71 (m, 1H), 1.70-1.62 (m, 1H), 1.57-1.42 (m, 1H), 0.93- |
| | 0.83 (m, 1H), 0.83-0.73 (m, 1H). |
| 17 | ¹H NMR (400 MHZ, DMSO-d₆ + 10% D₂O) δ 8.68 (s, 1H), 7.98 (dt, J = 6.8, |
| | 2.0 Hz, 1H), 7.78-7.64 (m, 2H), 7.31 (t, J = 7.6 Hz, 1H), 7.16 (d, J = 7.7 |
| | Hz, 2H), 6.31 (s, 1H), 5.78 (broad s, 1H), 4.57-4.43 (m, 1H), 3.87-3.71 |
| | (m, 2H), 3.71-3.52 (m, 2H), 3.51-3.34 (m, 4H), 3.34-3.18 (m, 1H), 2.07 |
| | (broad s, 6H), 1.84 (t, J = 6.0 Hz, 1H), 1.05-0.97 (m, 2H), 0.99-0.87 (m, |
| | 2H), 0.84-0.73 (m, 2H), 0.65-0.54 (m, 2H). |
| 18 | ¹H NMR (400 MHZ, DMSO-d₆ + 10% D₂O) δ 8.71 (s, 1H), 7.98 (dt, J = 7.0, |
| | 1.9 Hz, 1H), 7.80-7.65 (m, 2H), 7.31 (t, J = 7.6 Hz, 1H), 7.16 (d, J = 7.7 |
| | Hz, 2H), 6.32 (s, 1H), 5.76 (broad s, 1H), 4.57-4.45 (m, 1H), 3.90-3.76 |
| | (m, 3H), 3.76-3.62 (m, 1H), 3.55-3.39 (m, 2H), 3.39-3.16 (m, 3H), 2.06 |
| | (br s, 6H), 1.92-1.79 (m, 2H), 1.42 (t, J = 6.5 Hz, 1H), 0.95-0.84 (m, 4H), |
| | 0.74-0.65 (m, 2H), 0.60-0.49 (m, 2H). |
| 19 | ¹H NMR (400 MHZ, DMSO-d₆ + 10% D₂O) δ 8.63 (s, 1H), 8.03-7.90 (m, |
| | 1H), 7.79-7.65 (m, 2H), 7.31 (t, J = 7.6 Hz, 1H), 7.16 (d, J = 7.7 Hz, 2H), |
| | 6.27 (s, 1H), 5.82 (broad s, 1H), 4.35 (dt, J = 14.6, 6.8 Hz, 1H), 3.88-3.64 |
| | (m, 5H), 3.56-3.36 (m, 2H), 3.35-3.11 (m, 2H), 2.07 (br s, 6H), 1.11 (s, 9H). |

### Example 11: Preparation of Compound 35

### Step 1: (16R)-18-(Cyclopropylmethyl)-12-(2,6-dimethylphenyl)-8,8-dioxo-15-oxa-8λ⁶-thia-1,9,11,18,22-pentazatetracyclo[14.4.1.13,7.110,14]tricosa-3,5,7(23),10(22),11,13-hexaen-2-one (Compound 35)

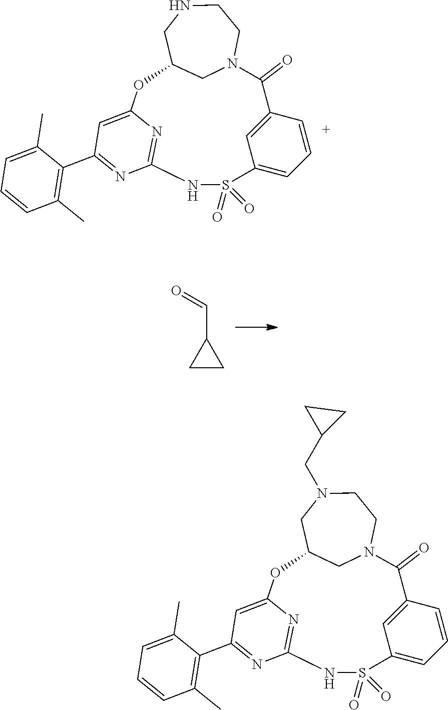 [SMILES: Cc1cccc(C)c1-c1cc2nc(n1)NS(=O)(=O)c1cccc(c1)C(=O)N1CCNC[C@H](C1)O2 ; O=CC1CC1 ; Cc1cccc(C)c1-c1cc2nc(n1)NS(=O)(=O)c1cccc(c1)C(=O)N1CCN(CC3CC3)C[C@H](C1)O2]

To a vial containing 12-(2,6-dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (10.5 mg, 0.02035 mmol) was added dichloroethane (700 μL), cyclopropanecarbaldehyde (7.7 μL, 0.1030 mmol), and acetic acid (6 μL, 0.1055 mmol). The reaction was stirred at rt for 1 h. Sodium cyanoborohydride (7 mg, 0.1114 mmol) was added and the reaction was allowed to stir at rt for 1 h. By LCMS analysis, the reaction was complete. The reaction was quenched with methanol, filtered, and purified by preparative HPLC using 1%-99% ACN:water with a 0.1% HCl modifier. (16R)-18-(Cyclopropylmethyl)-12-(2,6-dimethylphenyl)-8,8-dioxo-15-oxa-8λ⁶-thia-1,9,11,18,22-pentazatetracyclo[14.4.1.13,7.110,14]tricosa-3,5,7(23),10(22),11,13-hexaen-2-one (6.8 mg, 63%) ESI-MS m/z calc. 533.20966, found 534.4 (M+1)⁺; Retention time: 0.93 minutes was isolated as a white solid. ESI-MS m/z calc. 533.20966, found 534.4 (M+1)⁺; Retention time: 0.93 minutes; LC method A.

### Example 12: Preparation of Compound 36 and Compound 37

### Step 1: (16R)-18-(3,3-Dimethylcyclopentyl)-12-(2,6-dimethylphenyl)-15-oxa-8λ⁶-thia-1,9,11,18,22-pentaazatetracyclo[14.4.1.13,7.110,14]tricosa-3,5,7(23),10(22),11,13-hexaene-2,8,8-trione (mixture of diastereomers)

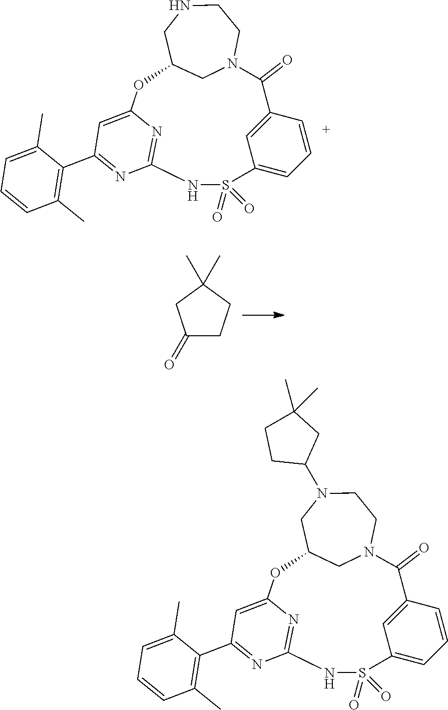

To a vial was added (16R)-12-(2,6-dimethylphenyl)-15-oxa-8λ⁶-thia-1,9,11,18,22-pentaazatetracyclo[14.4.1.13,7.110,14]tricosa-3,5,7(23),10,12,14(22)-hexaene-2,8,8-trione (hydrochloride salt) (20 mg, 0.03876 mmol), 3,3-dimethylcyclopentanone (approximately 21.74 mg, 0.1938 mmol), 5-ethyl-2-methylpyridine borane complex (approximately 15.70 mg, 17.31 μL, 0.1163 mmol), and acetic acid (approximately 46.55 mg, 44.08 μL, 0.7752 mmol). The reaction was heated at 30° C. and allowed to stir overnight. The reaction was quenched with methanol, filtered, and purified by preparative HPLC to give (16R)-18-(3,3-dimethylcyclopentyl)-12-(2,6-dimethylphenyl)-15-oxa-8λ⁶-thia-1,9,11,18,22-pentaazatetracyclo[14.4.1.13,7.110,14]tricosa-3,5,7(23),10(22),11,13-hexaene-2,8,8-trione (11.0 mg, 49%) as mixture of diastereomers.

### Step 2: (16R)-18-(3,3-Dimethylcyclopentyl)-12-(2,6-dimethylphenyl)-15-oxa-8λ⁶-thia-1,9,11,18,22-pentaazatetracyclo[14.4.1.13,7.110,14]tricosa-3,5,7(23),10(22),11,13-hexaene-2,8,8-trione, diastereomer 1 (Compound 36) and (16R)-18-(3,3-dimethylcyclopentyl)-12-(2,6-dimethylphenyl)-15-oxa-8λ⁶-thia-1,9,11,18,22-pentaazatetracyclo[14.4.1.13,7.110,14]tricosa-3,5,7(23),10(22),11,13-hexaene-2,8,8-trione, diastereomer 2 (Compound 37)

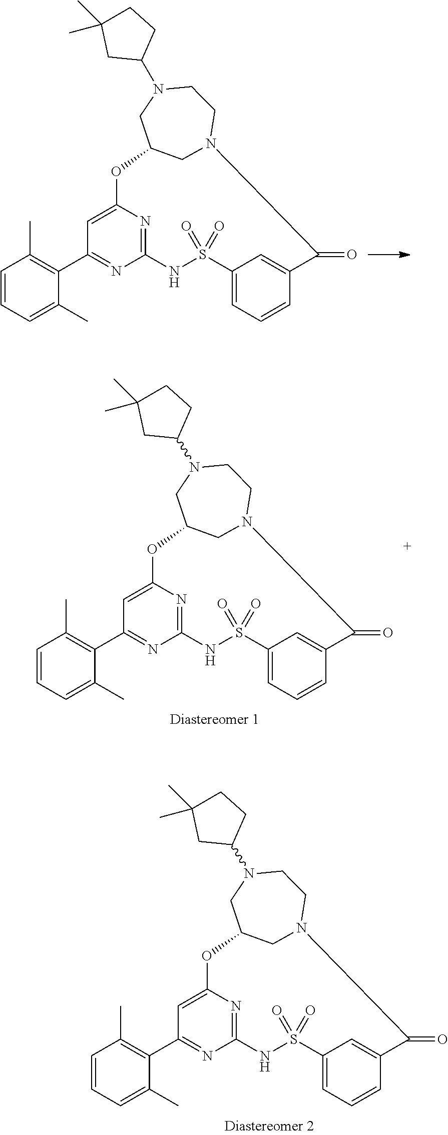 [SMILES: Cc1cccc(C)c1-c1cc2nc(n1)NS(=O)(=O)c1cccc(c1)C(=O)N1CCN(C3CCC(C)(C)C3)C[C@H](C1)O2 ; Cc1cccc(C)c1-c1cc2nc(n1)NS(=O)(=O)c1cccc(c1)C(=O)N1CCN(C3CCC(C)(C)C3)C[C@H](C1)O2 ; "+" ; Cc1cccc(C)c1-c1cc2nc(n1)NS(=O)(=O)c1cccc(c1)C(=O)N1CCN(C3CCC(C)(C)C3)C[C@H](C1)O2]

(16R)-18-(3,3-Dimethylcyclopentyl)-12-(2,6-dimethylphenyl)-15-oxa-8λ⁶-thia-1,9,11,18,22-pentaazatetracyclo[14.4.1.13,7.110,14]tricosa-3,5,7(23),10(22),11,13-hexaene-2,8,8-trione (11 mg, 0.01911 mmol) was subjected to chiral SFC separation (ChiralCel OD (250×10 mm) 5 m column, 35° C., mobile phase: 22% MeOH, 78% CO₂ (no modifier), flow: 10 mL/min, injection volume: 70 μL, pressure: 10 bar, wavelength: 210 nm) to provide as the first diastereomer to elute, (16R)-18-(3,3-dimethylcyclopentyl)-12-(2,6-dimethylphenyl)-15-oxa-8λ⁶-thia-1,9,11,18,22-pentaazatetracyclo[14.4.1.13,7.110,14]tricosa-3,5,7(23),10(22),11,13-hexaene-2,8,8-trione (hydrochloride salt) (2.9 mg, 50%). ESI-MS m/z calc. 575.25665, found 576.3 (M+1)⁺; Retention time: 1.19 minutes; and as the second diastereomer to elute (16R)-18-(3,3-dimethylcyclopentyl)-12-(2,6-dimethylphenyl)-15-oxa-8λ⁶-thia-1,9,11,18,22-pentaazatetracyclo[14.4.1.13,7.110,14]tricosa-3,5,7(23),10(22),11,13-hexaene-2,8,8-trione (hydrochloride salt) (3.4 mg, 58%). ESI-MS m/z calc. 575.25665, found 576.5 (M+1)⁺; Retention time: 1.2 minutes (LC method A).

### Example 13: Preparation of Compound 38, Compound 39, and Compound 40

### Step 1: (16R)-12-(2,6-Dimethylphenyl)-18-(4-fluorocyclohexyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione, 2:1 diastereomeric mixture (Compound 40), (16R)-12-(2,6-dimethylphenyl)-18-(4-fluorocyclohexyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione, diastereomer 1 (Compound 38), and (16R)-12-(2,6-dimethylphenyl)-18-(4-fluorocyclohexyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione, diastereomer 2 (Compound 39)

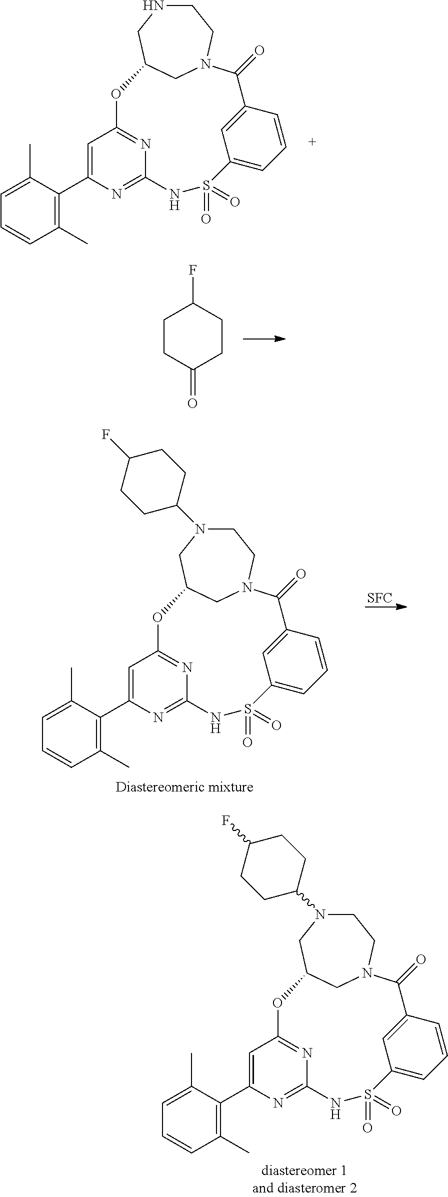

In a 4 mL vial, to a stirred solution of 4-fluorocyclohexanone (35 mg, 0.3014 mmol) in anhydrous 1,2-dichloroethane (1.5 mL) were added (16R)-12-(2,6-dimethylphenyl)-15-oxa-8λ⁶-thia-1,9,11,18,22-pentaazatetracyclo[14.4.1.13,7.110,14]tricosa-3,5,7(23),10,12,14(22)-hexaene-2,8,8-trione (hydrochloride salt) (40 mg, 0.07752 mmol), triethylamine (20 μL, 0.1435 mmol), and glacial acetic acid (10 μL, 0.1758 mmol), in that order. The resulting light-yellow solution was stirred at ambient temperature for 30 min, then sodium cyanoborohydride (40 mg, 0.6365 mmol) was added and stirring continued for 13 h. The crude material was diluted with DMSO (0.8 mL), microfiltered, and purified by preparative reverse-phase HPLC eluting with 1-99% acetonitrile in water over 15 min (HCl as modifier). The desired product (16R)-12-(2,6-dimethylphenyl)-18-(4-fluorocyclohexyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione (hydrochloride salt) (33 mg, 69%) obtained as diastereomeric mixture as a white solid. ESI-MS m/z calc. 579.23157, found 580.5 (M+1)⁺; Retention time: 1.01 minutes (LC method A).

Two diastereomers were separated by preparative SFC (Column: ChiralCel OD (250×10 mm), 5 μm; 35 C; Mobile phase: 30% MeOH (No Mod), 70% CO2; Flow: 10 mL/min; Concentrations: ˜ 23 mg/mL in MeOH (No Mod); Injection Volume 70 μL; Pressure: 179 bar; Wavelength: 210 nm) to give: Peak 1, diastereomer 1, (16R)-12-(2,6-dimethylphenyl)-18-(4-fluorocyclohexyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione (17.5 mg, 39%). ¹H NMR (500 MHz, Methanol-d₄) δ 8.63 (t, J=1.7 Hz, 1H), 7.99 (dt, J=7.2, 1.8 Hz, 1H), 7.74-7.60 (m, 2H), 7.27 (t, J=7.7 Hz, 1H), 7.14 (d, J=7.7 Hz, 2H), 6.18 (s, 1H), 5.61 (tt, J=9.4, 4.7 Hz, 1H), 4.82-4.67 (m, 1H), 4.27 (ddd, J=14.3, 8.7, 5.9 Hz, 1H), 3.66 (dd, J=14.5, 4.0 Hz, 1H), 3.38 (dd, J=13.1, 5.2 Hz, 1H), 3.30-3.25 (m, 2H), 3.25-3.18 (m, 1H), 3.00 (dd, J=13.1, 9.3 Hz, 1H), 2.95 (ddd, J=13.5, 8.7, 4.6 Hz, 1H), 2.76 (dt, J=10.9, 6.4 Hz, 1H), 2.39-1.93 (m, 8H), 1.83-1.72 (m, 3H), 1.72-1.51 (m, 3H). ESI-MS m/z calc. 579.23157, found 580.4 (M+1)⁺; Retention time: 0.97 minutes (LC method A); and peak 2, diastereomer 2, (16R)-12-(2,6-dimethylphenyl)-18-(4-fluorocyclohexyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione (9 mg, 20%)¹H NMR (500 MHz, Methanol-d₄) δ 8.61 (d, J=1.8 Hz, 1H), 7.99 (dt, J=7.0, 1.9 Hz, 1H), 7.71-7.62 (m, 2H), 7.27 (t, J=7.7 Hz, 1H), 7.14 (d, J=7.7 Hz, 2H), 6.17 (s, 1H), 5.59 (tt, J=9.6, 4.7 Hz, 1H), 4.56-4.37 (m, 1H), 4.31-4.23 (m, 1H), 3.65 (dd, J=14.4, 4.0 Hz, 1H), 3.35 (dd, J=13.3, 5.3 Hz, 1H), 3.29-3.23 (m, 2H), 3.22-3.16 (m, 1H), 3.01-2.87 (m, 2H), 2.77-2.69 (m, 1H), 2.33-2.00 (m, 8H), 1.99-1.90 (m, 2H), 1.61-1.42 (m, 4H). ESI-MS m/z calc. 579.23157, found 580.4 (M+1)⁺; Retention time: 0.98 minutes (LC method A).

### Example 14: Preparation of Compound 41

### Step 1: (16R)-12-(2,6-Dimethylphenyl)-18-{2-oxaspiro[3.5]nonan-7-yl}-15-oxa-8λ⁶-thia-1,9,11,18,22-pentaazatetracyclo[14.4.1.13,7.110,14]tricosa-3(23),4,6,10,12,14(22)-hexaene-2,8,8-trione (Compound 41)

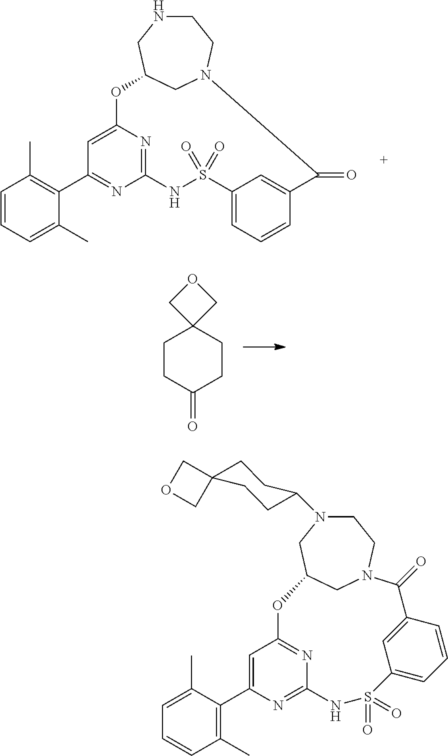

A 4 mL vial was charged with (16R)-12-(2,6-dimethylphenyl)-15-oxa-8λ⁶-thia-1,9,11,18,22-pentaazatetracyclo[14.4.1.13,7.110,14]tricosa-3,5,7(23),10,12,14(22)-hexaene-2,8,8-trione (hydrochloride salt) (15 mg, 0.02907 mmol), anhydrous DCM (1 mL), N,N-diisopropyl ethyl amine (10 μL, 0.05741 mmol), 2-oxaspiro[3.5]nonan-7-one (22 mg, 0.1569 mmol) and glacial acetic acid (10 μL, 0.1758 mmol). The vial was briefly purged with nitrogen, capped and stirred at room temperature for about 10 minutes. Sodium triacetoxyborohydride (25 mg, 0.1180 mmol) was added. The vial was purged with nitrogen, capped and the reaction was stirred at room temperature for 13 hours (overnight). Methanol (0.25 mL) was added. The volatiles were evaporated under reduced pressure and the residue was taken in DMSO (1 mL). The solution was microfiltered (0.45 uM) and purified from reverse phase preparative HPLC (C₁₈) using a gradient of acetonitrile in water (1 to 99% over 15 min, HCl as a modifier) to give as a white solid. (16R)-12-(2,6-dimethylphenyl)-18-{2-oxaspiro[3.5]nonan-7-yl}-15-oxa-8λ⁶-thia-1,9,11,18,22-pentaazatetracyclo[14.4.1.13,7.110,14]tricosa-3(23),4,6,10,12,14(22)-hexaene-2,8,8-trione (hydrochloride salt) (4.4 mg, 23%) ¹H NMR (499 MHz, Methanol-d₄) δ 8.91 (s, 1H), 8.06 (dt, J=7.7, 1.5 Hz, 1H), 7.76 (dt, J=7.7, 1.5 Hz, 1H), 7.71 (t, J=7.7 Hz, 1H), 7.28 (t, J=7.7 Hz, 1H), 7.15 (d, J=7.7 Hz, 2H), 6.29 (s, 1H), 6.14-6.04 (m, 1H), 4.73-4.55 (m, 1H), 4.07-3.98 (m, 1H), 3.93 (d, J=7.7 Hz, 2H), 3.85 (dt, J=13.4, 6.6 Hz, 1H), 3.75-3.66 (m, 1H), 3.63 (s, 2H), 3.60-3.48 (m, 2H), 3.41 (s, 2H), 3.38-3.32 (m, 1H), 2.11 (s, 6H), 2.03 (d, J=10.3 Hz, 2H), 1.94-1.73 (m, 4H), 1.37 (t, J=13.3 Hz, 1H), 1.11 (t, J=16.7 Hz, 1H). ESI-MS m/z calc. 603.2515, found 604.4 (M+1)⁺; Retention time: 0.92 minutes (LC method A).

### Example 15: Preparation of Compound 42 and Compound 43

### Step 1: 1,4-Dibenzyl-1,4-diazepan-6-ol

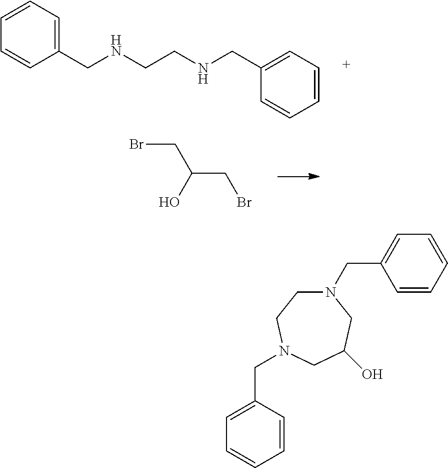 [SMILES: c1ccc(CNCCNCc2ccccc2)cc1 ; OC(CBr)CBr ; OC1CN(Cc2ccccc2)CCN(Cc2ccccc2)C1]

To a solution of N,N′-dibenzylethane-1,2-diamine (49.97 g, 48.990 mL, 205.83 mmol) in toluene (1.2 L) was added 1,3-dibromopropan-2-ol (45.3 g, 21.268 mL, 197.51 mmol) and triethyl amine (59.95 g, 82.576 mL, 592.45 mmol) slowly. The solution was refluxed for 2 days. The solvent was removed and the residue was dissolved into water (400 mL), extracted with ethyl acetate (300 mL×3). The combined organic layer was washed with water, brine, and dried with sodium sulfate, filtered and concerted under reduce pressure. The residue was purified by silica gel chromatography using gradient method ethyl acetate/hexane 0-100% to afford 1,4-dibenzyl-1,4-diazepan-6-ol (27 g, 42%) as a yellow oil. ¹H NMR (250 MHz, CDCl₃) δ 7.40-7.18 (m, 10H), 3.81 (p, J=3.7 Hz, 1H), 3.72-3.64 (s, 4H), 2.90-2.66 (m, 6H), 2.47 (tdd, J=8.1, 6.7, 5.0 Hz, 2H). ESI-MS m/z calc. 296.18887, found 297.2 (M+1)⁺; Retention time: 1.53 minutes; LC method T.

### Step 2: tert-Butyl 6-hydroxy-1,4-diazepane-1-carboxylate

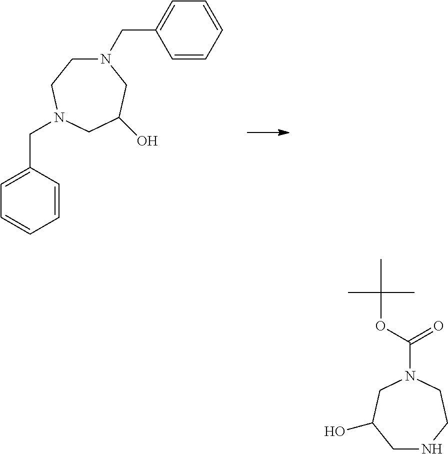 [SMILES: OC1CN(Cc2ccccc2)CCN(Cc2ccccc2)C1 ; CC(C)(C)OC(=O)N1CCNCC(O)C1]

A solution of 1,4-dibenzyl-1,4-diazepan-6-ol (13.36 g, 45.07 mmol) in methanol (500 mL) was purged under nitrogen gas. Palladium hydroxide (3.03 g of 20% on carbon, 50% wet, 2.16 mmol) was added, the reaction mixture was purged under hydrogen gas then left to stir under one atmosphere of hydrogen for 24 hours. The reaction mixture was once again purged under nitrogen gas, then filtered over Celite and washed with methanol (about 500 mL). Concentrated under reduced pressure to afford the crude diamine as a yellow oil. The crude diamine was dissolved in methanol (200 mL) and cooled in an ice bath. Triethylamine (7.6 mL, 54.5 mmol) was added, followed by di-tert-butyl dicarbonate (9.85 g, 45.1 mmol) and the reaction was left to gradually warm to room temperature and stir overnight. The reaction mixture was concentrated under reduced pressure, then suspended in dichloromethane (about 150 mL) and heptanes (about 100 mL). A white fluffy solid crashed out. The solid was filtered off and the filtrate was absorbed on silica gel and purified by silica gel chromatography on a 220-g column, eluting from 0% to 10% methanol in dichloromethane to afford tert-butyl 6-hydroxy-1,4-diazepane-1-carboxylate (3.265 g, 32%) as a thick amber oil. ¹H NMR (300 MHz, CDCl₃) δ 1.47 (s, 9H), 2.69-3.12 (m, 6H), 3.14-3.80 (m, 4H), 3.91-4.05 (m, 1H). ESI-MS m/z calc. 216.1474, found 217.2 (M+1)⁺; Retention time: 0.93 minutes (LC method M).

### Step 3: 4-{3-[4-Chloro-6-(2,6-dimethyl-phenyl)-pyrimidin-2-ylsulfamoyl]-benzoyl}-6-hydroxy-[1,4]diazepane-1-carboxylic acid tert-butyl ester

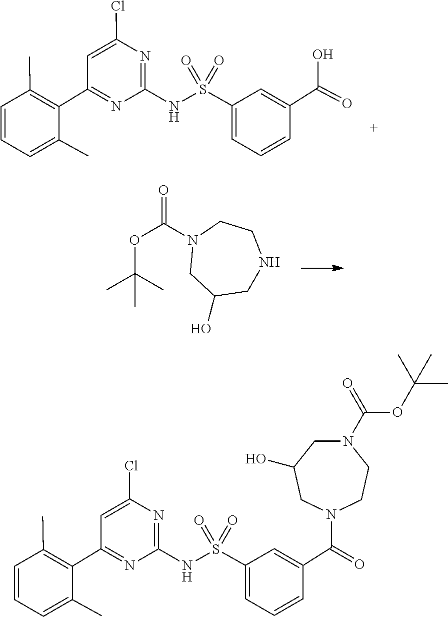 [SMILES: Cc1cccc(C)c1-c1cc(Cl)nc(NS(=O)(=O)c2cccc(C(=O)O)c2)n1 ; CC(C)(C)OC(=O)N1CCNCC(O)C1 ; Cc1cccc(C)c1-c1cc(Cl)nc(NS(=O)(=O)c2cccc(C(=O)N3CCN(C(=O)OC(C)(C)C)CC(O)C3)c2)n1]

To a solution of 3-[4-chloro-6-(2,6-dimethyl-phenyl)-pyrimidin-2-ylsulfamoyl]-benzoic acid (9.82 g, 23.5 mmol) in dichloromethane (150 mL) was added N,N′-diisopropylcarbodiimide (3.68 mL, 23.5 mmol) at room temperature. The reaction mixture was stirred for 10 minutes. A solution of 6-hydroxy-[1,4]diazepane-1-carboxylic acid tert-butyl ester (4.53 g, 20.95 mmol) in dichloromethane (75 mL) was added at room temperature dropwise within 1 hour. The reaction was stirred for another 30 minutes, and then it was quenched with a 10% aqueous citric acid solution (75 mL). The two layers were separated. The aqueous layer was extracted with dichloromethane (2×150 mL) and the combined organic layers were washed with brine (100 mL), dried over anhydrous sodium sulfate and concentrated. The residue was purified by silica gel column chromatography using 0-80% hexanes-acetone to furnish 4-{3-[4-chloro-6-(2,6-dimethyl-phenyl)-pyrimidin-2-ylsulfamoyl]-benzoyl}-6-hydroxy-[1,4]diazepane-1-carboxylic acid tert-butyl ester (7.62 g, 59%) as a pink solid. ESI-MS m/z: calc. 615.19, found 616.0 (M1). Retention time: 5.24 minutes.

### Step 4: tert-Butyl 12-(2,6-dimethylphenyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (Compound 43)

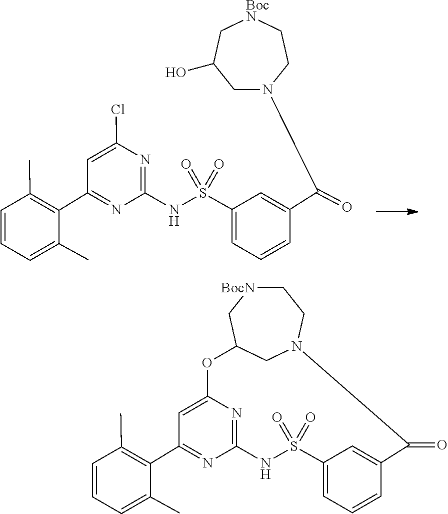

To a solution of 4-{3-[4-chloro-6-(2,6-dimethyl-phenyl)-pyrimidin-2-ylsulfamoyl]-benzoyl}-6-hydroxy-[1,4]diazepane-1-carboxylic acid tert-butyl ester (7.62 g, 12.37 mmol) in anhydrous dimethylformamide (800 mL) was added a 60% suspension sodium hydride in mineral oil (4.95 g, 123.7 mmol) in several portions. The reaction mixture was stirred at room temperature for 16 hours and then quenched with 10% aqueous citric acid solution (500 mL). The product was extracted with ethyl acetate (3×500 mL) and the combined organic layers were washed with brine (3×500 mL), dried over anhydrous sodium sulfate and concentrated. The residue was purified by silica gel column chromatography using 0-70% hexanes-acetone to furnish tert-butyl 16-(2,6-dimethylphenyl)-4-oxo-2-oxa-6-thia-7-aza-3(6,1)-diazepana-1(4,2)-pyrimidina-5(1,3)-benzenacycloheptaphane-34-carboxylate 6,6-dioxide (4.404 g, 56%) as a white solid. 1H-NMR (250 MHz, DMSO-d₆) δ (ppm): 8.30 (d, J=15.1 Hz, 1H), 7.92 (s, 1H), 7.67 (s, 2H), 7.27 (m, 1H), 7.14 (m, 2H), 6.35 (s, 1H), 5.50 (m, 1H), 4.48 (m, 1H), 3.99 (m, 2H), 3.56 (m, 1H), 3.24 (m, 5H), 2.05 (s, 6H), 1.42 (d, J=10.5 Hz, 9H). ESI-MS m/z calc. 579.21515, found 580.2 (M+1)⁺; Retention time: 4.66 minutes.

### Step 5: 12-(2,6-Dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 42)

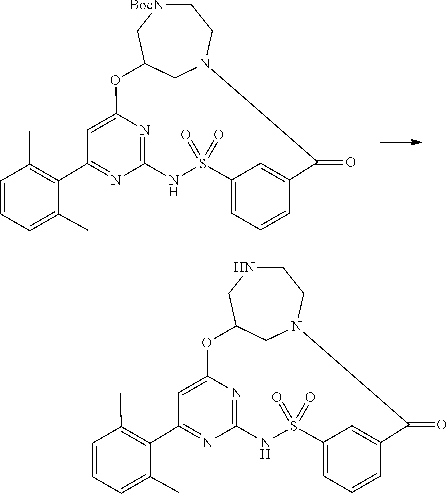

TFA (12 mL, 155.8 mmol) was added to 16-(2,6-dimethylphenyl)-2-oxa-6-thia-7-aza-3(6,1)-diazepana-1(4,2)-pyrimidina-5(1,3)-benzenacycloheptaphan-4-one 6,6-dioxide (3 g, 5.175 mmol) in DCM (50 mL). The mixture was stirred at room temperature. Solvents were removed and the crude was resuspended in DCM/toluene and the mixture was concentrated to dryness under reduced pressure (this step was repeated 3 times) to give 12-(2,6-dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (2.3 g, 93%). ¹H NMR (400 MHz, DMSO-d₆) δ 10.42 (s, 1H), 9.46 (s, 1H), 8.76 (s, 1H), 7.95 (s, 1H), 7.67 (s, 2H), 7.28 (s, 1H), 7.14 (s, 2H), 6.38 (s, 1H), 5.75 (s, 1H), 4.49 (s, 1H), 3.76 (s, 2H), 3.62 (s, 1H), 3.43 (s, 3H), 3.25 (s, 1H), 2.05 (s, 6H). ESI-MS m/z calc. 479.16272, found 480.0 (M+1)⁺; Retention time: 0.69 minutes; LC method A.

### Example 16: Preparation of Compound 44

### Step 1: 12-(2,6-Dimethylphenyl)-18-isobutyl-8,8-dioxo-15-oxa-8λ⁶-thia-1,9,11,18,22-pentazatetracyclo[14.4.1.13,7.110,14]tricosa-3,5,7(23),10(22),11,13-hexaen-2-one (Compound 44)

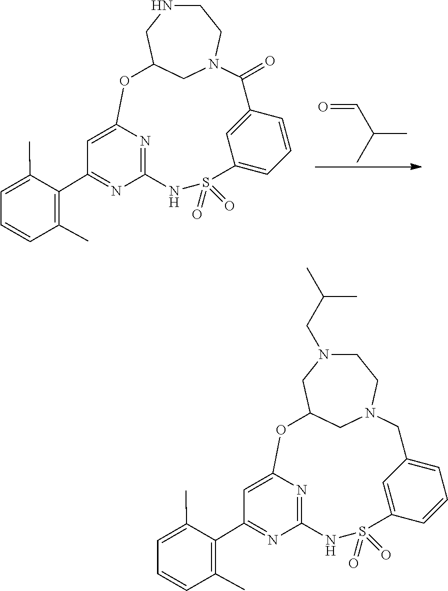

2-Methylpropanal (approximately 22.53 mg, 0.3125 mmol) was added to 12-(2,6-Dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (30 mg, 0.06250 mmol) in Acetic acid (0.5 mL) in a 3 ml vial, followed by sodium triacetoxy borohydride (approximately 132.5 mg, 0.6250 mmol). This reaction mixture was stirred at room temperature for 1 h, then at 60° C. for 16 h. It was then cooled to room temperature, filtered and purified by reverse phase PLC to give 12-(2,6-dimethylphenyl)-18-isobutyl-8,8-dioxo-15-oxa-8λ⁶-thia-1,9,11,18,22-pentazatetracyclo[14.4.1.13,7.110,14]tricosa-3,5,7(23),10(22),11,13-hexaen-2-one (4.3 mg, 13%). ESI-MS m/z calc. 535.22534, found 536.0 (M+1)⁺; Retention time: 1.0 minutes; LC method A.

### Example 17: Characterization of Compounds 45-63

The compounds in the following tables were prepared in a manner analogous to that described above using commercially available reagents and intermediates described herein.

TABLE 5

| Compound | | LCMS | Calc. | | LCMS |
|---|---|---|---|---|---|
| number | Structure | Rt (min) | mass | M + 1 | Method |
| 45 | 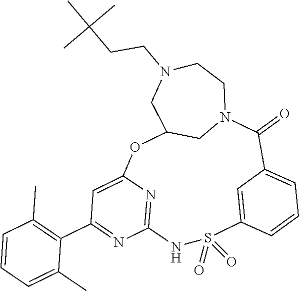 [SMILES: Cc1cccc(C)c1-c1cc2nc(n1)NS(=O)(=O)c1cccc(c1)C(=O)N1CCN(CCC(C)(C)C)CC(C1)O2] | 1.18 | 563.257 | 564.6 | A |
| 46 | 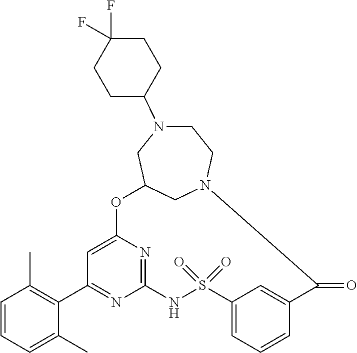 [SMILES: Cc1cccc(C)c1-c1cc2nc(n1)NS(=O)(=O)c1cccc(c1)C(=O)N1CCN(C3CCC(F)(F)CC3)CC(C1)O2] | 1.08 | 597.222 | 598.38 | A |
| 47 | 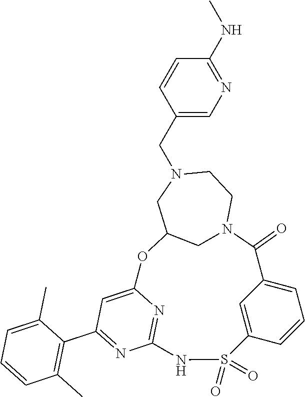 [SMILES: CNc1ccc(CN2CCN3CC(C2)Oc2cc(-c4c(C)cccc4C)nc(n2)NS(=O)(=O)c2cccc(c2)C3=O)cn1] | 0.93 | 599.231 | 600 | A |
| 48 | 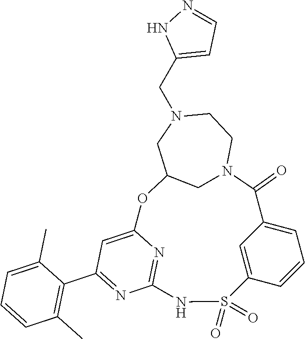 [SMILES: Cc1cccc(C)c1-c1cc2nc(n1)NS(=O)(=O)c1cccc(c1)C(=O)N1CCN(Cc3ccn[nH]3)CC(C1)O2] | 0.87 | 559.2 | 560 | A |
| 49 | 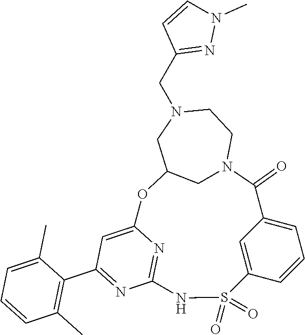 [SMILES: Cc1cccc(C)c1-c1cc2nc(n1)NS(=O)(=O)c1cccc(c1)C(=O)N1CCN(Cc3ccn(C)n3)CC(C1)O2] | 0.92 | 573.216 | 574 | A |
| 50 | 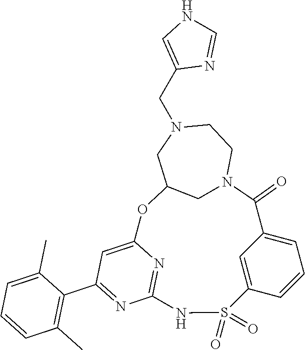 [SMILES: Cc1cccc(C)c1-c1cc2nc(n1)NS(=O)(=O)c1cccc(c1)C(=O)N1CCN(Cc3c[nH]cn3)CC(C1)O2] | 0.96 | 559.2 | 560 | A |
| 51 | 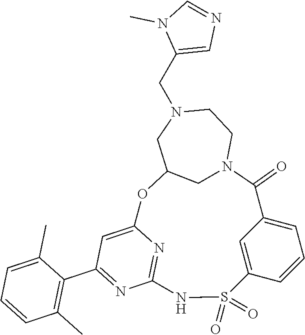 [SMILES: Cc1cccc(C)c1-c1cc2nc(n1)NS(=O)(=O)c1cccc(c1)C(=O)N1CCN(Cc3cncn3C)CC(C1)O2] | 1.02 | 573.216 | 574 | A |
| 52 | 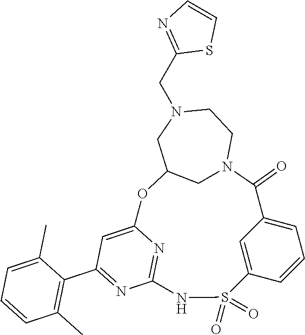 [SMILES: Cc1cccc(C)c1-c1cc2nc(n1)NS(=O)(=O)c1cccc(c1)C(=O)N1CCN(Cc3nccs3)CC(C1)O2] | 1.23 | 576.161 | 577 | A |
| 53 | 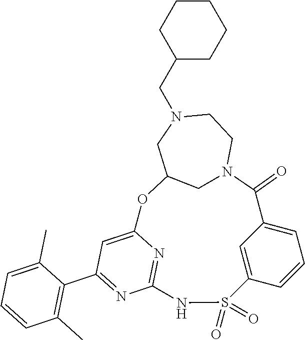 [SMILES: Cc1cccc(C)c1-c1cc2nc(n1)NS(=O)(=O)c1cccc(c1)C(=O)N1CCN(CC3CCCCC3)CC(C1)O2] | 1.19 | 575.257 | 576 | A |
| 54 | 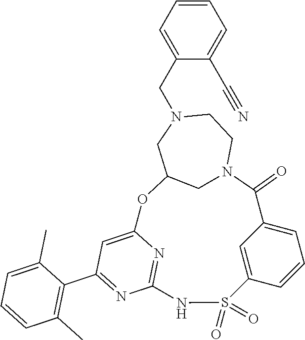 [SMILES: Cc1cccc(C)c1-c1cc2nc(n1)NS(=O)(=O)c1cccc(c1)C(=O)N1CCN(Cc3ccccc3C#N)CC(C1)O2] | 1.48 | 594.205 | 595 | A |
| 55 | 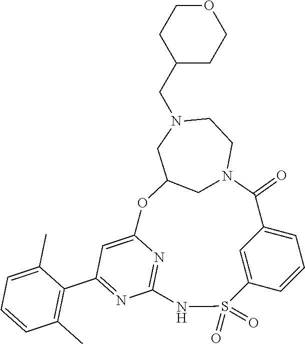 [SMILES: Cc1cccc(C)c1-c1cc2nc(n1)NS(=O)(=O)c1cccc(c1)C(=O)N1CCN(CC3CCOCC3)CC(C1)O2] | 0.93 | 577.236 | 578 | A |
| 56 | 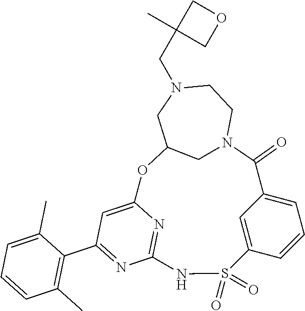 [SMILES: Cc1cccc(C)c1-c1cc2nc(n1)NS(=O)(=O)c1cccc(c1)C(=O)N1CCN(CC3(C)COC3)CC(C1)O2] | 1.07 | 563.22 | 564 | A |
| 57 | 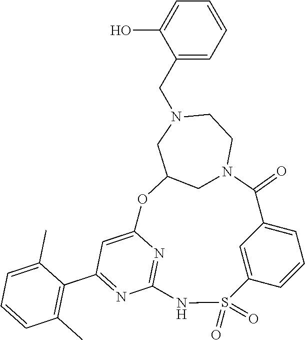 [SMILES: Cc1cccc(C)c1-c1cc2nc(n1)NS(=O)(=O)c1cccc(c1)C(=O)N1CCN(Cc3ccccc3O)CC(C1)O2] | 1.06 | 585.205 | 586 | A |
| 58 | 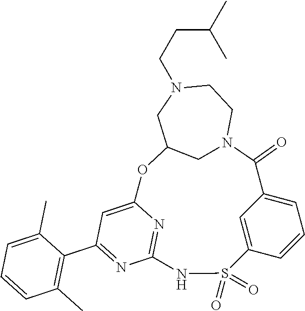 [SMILES: Cc1cccc(C)c1-c1cc2nc(n1)NS(=O)(=O)c1cccc(c1)C(=O)N1CCN(CCC(C)C)CC(C1)O2] | 1.12 | 549.241 | 550 | A |
| 59 | 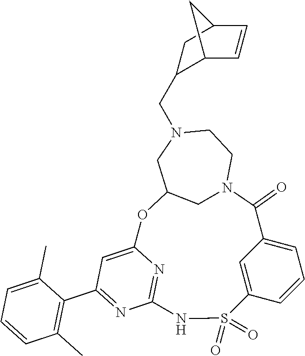 [SMILES: Cc1cccc(C)c1-c1cc2nc(n1)NS(=O)(=O)c1cccc(c1)C(=O)N1CCN(CC3CC4C=CC3C4)CC(C1)O2] | 1.16 | 585.241 | 586 | A |
| 60 | 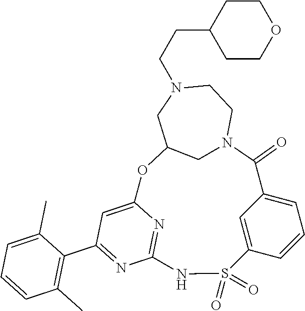 [SMILES: Cc1cccc(C)c1-c1cc2nc(n1)NS(=O)(=O)c1cccc(c1)C(=O)N1CCN(CCC3CCOCC3)CC(C1)O2] | 0.97 | 591.252 | 592 | A |
| 61 | 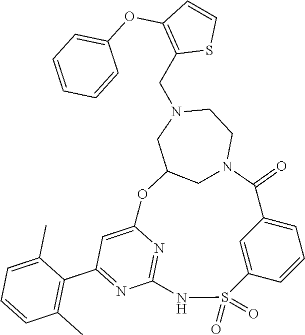 [SMILES: Cc1cccc(C)c1-c1cc2nc(n1)NS(=O)(=O)c1cccc(c1)C(=O)N1CCN(Cc3sccc3Oc3ccccc3)CC(C1)O2] | 1.54 | 667.192 | 668 | A |
| 62 | 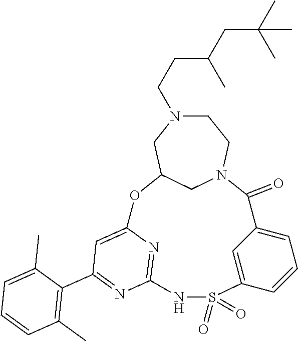 [SMILES: Cc1cccc(C)c1-c1cc2nc(n1)NS(=O)(=O)c1cccc(c1)C(=O)N1CCN(CCC(C)CC(C)(C)C)CC(C1)O2] | 1.47 | 605.304 | 606 | A |
| 63 | 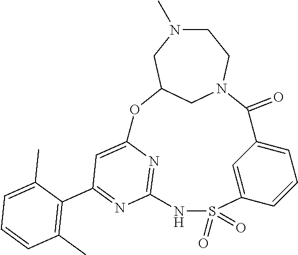 [SMILES: Cc1cccc(C)c1-c1cc2nc(n1)NS(=O)(=O)c1cccc(c1)C(=O)N1CCN(C)CC(C1)O2] | 0.82 | 493.178 | 494 | A |

TABLE 6

| Com- | |
|---|---|
| pound | |
| number | NMR |
| 46 | ¹H NMR (400 MHZ, DMSO-d₆ + 10% D₂O) δ 8.71 (s, 1H), 8.01-7.93 (m, |
| | 1H), 7.76-7.68 (m, 2H), 7.30 (t, J = 7.6 Hz, 1H), 7.16 (d, J = 7.7 Hz, 2H), |
| | 6.30 (s, 1H), 5.84-5.71 (m, 1H), 4.53-4.39 (m, 1H), 3.86-3.77 (m, 3H), |
| | 3.77-3.55 (m, 3H), 3.48 (dt, J = 13.6, 5.9 Hz, 1H), 3.29 (dd, J = 14.4, 11.0 |
| | Hz, 1H), 2.32-1.72 (m, 14H). Sulfonamide NH visible as a broad singlet |
| | at 10.70 ppm in the absence of D₂O. |
| 53 | ¹H NMR (400 MHZ, DMSO-d₆) δ 11.62 (s, 1H), 9.91 (s, 1H), 7.61 (s, 1H), |
| | 7.48-7.31 (m, 8H), 7.05 (s, 1H), 3.75 (s, 3H), 3.51 (d, J = 11.9 Hz, 2H), |
| | 3.08 (s, 2H), 2.91 (s, 1H), 2.80 (s, 3H), 2.26 (s, 3H), 1.95 (d, J = 13.1 Hz, |
| | 4H). |
| 63 | ¹H NMR (400 MHZ, DMSO-d₆) δ 10.68 (s, 1H), 8.80 (s, 1H), 7.94 (s, 1H), |
| | 7.69 (s, 2H), 7.40-7.05 (m, 4H), 6.42 (s, 1H), 5.81 (s, 1H), 4.55 (s, 1H), |
| | 3.96 (s, 1H), 3.81 (d, J = 14.0 Hz, 2H), 3.73-3.62 (m, 2H), 3.42 (s, 2H), |
| | 3.33-3.18 (m, 2H), 3.04 (s, 3H), 2.70 (s, 1H), 2.18 (s, 1H), 2.04 (s, 6H), |
| | 1.90 (qd, J = 8.0, 6.6 Hz, 1H). |

### Example 18: Preparation of Compound 64

### Step 1: 12-(2,6-Dimethylphenyl)-18-[(pyridin-2-yl)methyl]-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 64) and 12-(2,6-dimethylphenyl)-18-[(pyridin-4-yl)methyl]-15-oxa-8λ⁶-thia-1,9,11,18,22-pentaazatetracyclo[14.4.1.13,7.110,14]tricosa-3(23),4,6,10(22),11,13-hexaene-2,8,8-trione

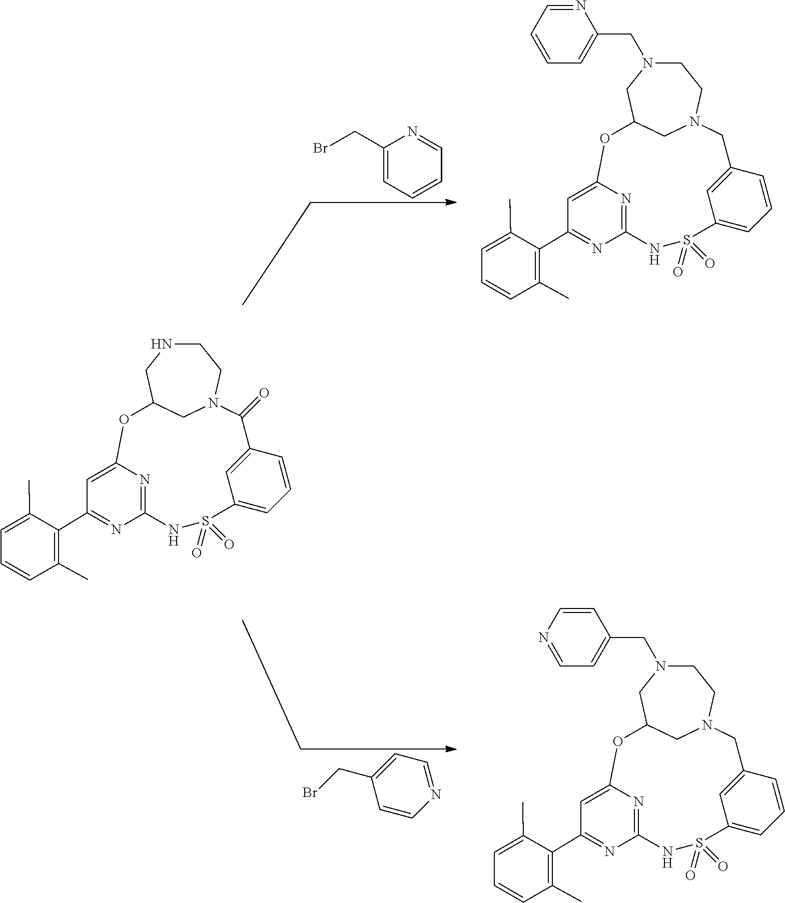 [SMILES: BrCc1ccccn1 ; Cc1cccc(C)c1-c1cc2nc(n1)NS(=O)(=O)c1cccc(c1)CN1CCN(Cc3ccccn3)CC(C1)O2 ; Cc1cccc(C)c1-c1cc2nc(n1)NS(=O)(=O)c1cccc(c1)C(=O)N1CCNCC(C1)O2 ; BrCc1ccncc1 ; Cc1cccc(C)c1-c1cc2nc(n1)NS(=O)(=O)c1cccc(c1)CN1CCN(Cc3ccncc3)CC(C1)O2]

Two reaction were run in separated vials: 12-(2,6-Dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (20 mg, 0.04166 mmol), 2-(bromomethyl)pyridine (hydrobromide salt) (15 mg, 0.05930 mmol), TEA (35 μL, 0.2511 mmol), and DMF (0.5 mL) were combined and stirred at 90° C. for 16 h. The reaction mixture was filtered and purified on reverse phase HPLC (Waters, HCl, 10-60% ACN-water) to give 12-(2,6-dimethylphenyl)-18-[(pyridin-2-yl)methyl]-15-oxa-8λ⁶-thia-1,9,11,18,22-pentaazatetracyclo[14.4.1.13,7.110,14]tricosa-3(23),4,6,10(22),11,13-hexaene-2,8,8-trione (8.5 mg, 36%). ¹H NMR (400 MHz, DMSO-d₆) δ 8.77 (d, J=9.7 Hz, 2H), 8.10-8.03 (m, 1H), 7.94 (s, 1H), 7.70 (d, J=11.8 Hz, 3H), 7.63-7.56 (m, 1H), 7.26 (t, J=7.6 Hz, 1H), 7.13 (d, J=7.6 Hz, 2H), 6.37 (s, 1H), 5.89 (s, 1H), 4.72 (s, 2H), 4.40 (s, 1H), 3.95-3.91 (m, 3H), 3.42 (s, 3H), 3.30 (s, 1H), 2.70 (s, 1H), 2.20 (s, 1H), 2.05 (s, 6H), 1.90 (s, 1H). ESI-MS m/z calc. 570.2049, found 571.0 (M+1)⁺; Retention time: 0.96 minutes (LC method A).

In a second vial, 12-(2,6-Dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (20 mg, 0.04166 mmol), 4-(bromomethyl)pyridine (hydrobromide salt) (15 mg, 0.05930 mmol), TEA (35 μL, 0.2511 mmol), and DMF (0.5 mL) were combined and stirred at 90° C. for 16 h. The reaction mixture was filtered and purified on reverse phase HPLC (Waters, HCl, 10-60% ACN-water) to give 12-(2,6-dimethylphenyl)-18-[(pyridin-4-yl)methyl]-15-oxa-8λ⁶-thia-1,9,11,18,22-pentaazatetracyclo[14.4.1.13,7.110,14]tricosa-3(23),4,6,10(22),11,13-hexaene-2,8,8-trione ESI-MS m/z calc. 570.2049, found 571.0 (M+1)⁺; Retention time: 0.99 minutes (LC method A).

### Example 19: Preparation of Compound 65

### Step 1: 18-(4,4-Dimethylpentyl)-12-(2,6-dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 65)

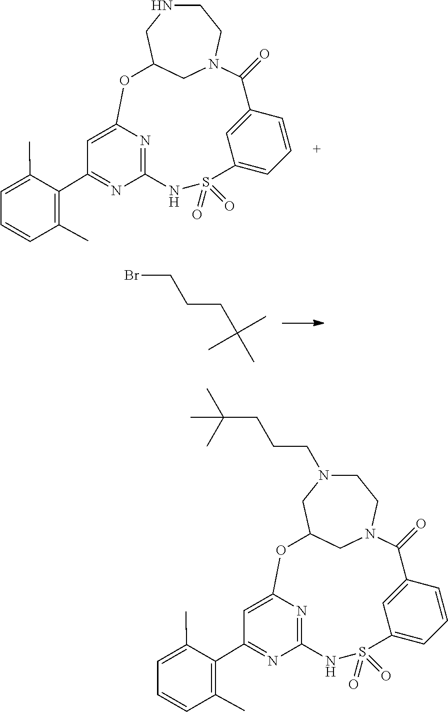

12-(2,6-Dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (30 mg, 0.06250 mmol), 1-bromo-4,4-dimethyl-pentane (16 mg, 0.08934 mmol), triethyl amine (25 mg, 0.2471 mmol), and DMF (0.5 mL) were combined and stirred at 110° C. for 16 h. The reaction mixture was filtered and purified on reverse phase HPLC (Waters, HCl, 25-75% ACN-water) to give 18-(4,4-dimethylpentyl)-12-(2,6-dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (2 mg, 6%) ESI-MS m/z calc. 577.2723, found 578.0 (M+1)⁺; Retention time: 1.26 minutes (LC method A).

### Example 20: Preparation of Compound 66

### Step 1: (36R)-16-(2,6-dimethylphenyl)-34-(pyridin-3-ylmethyl)-2-oxa-6-thia-7-aza-3(6,1)-diazepana-1(4,2)-pyrimidina-5(1,3)-benzenacycloheptaphan-4-one 6,6-dioxide (Compound 66)

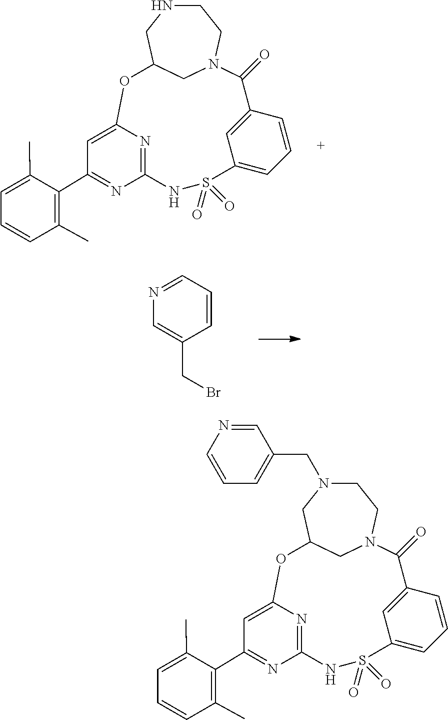

12-(2,6-Dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (20 mg, 0.04166 mmol), 3-(bromomethyl)pyridine (hydrobromide salt) (approximately 15.81 mg, 0.06249 mmol), TEA (approximately 16.86 mg, 23.22 μL, 0.1666 mmol), and DMF (1 mL) were combined and stirred at 120° C. for 16 h. The reaction mixture was filtered and purified on reverse phase HPLC (Waters, HCl, 25-75% ACN-water) to give (36R)-16-(2,6-dimethylphenyl)-34-(pyridin-3-ylmethyl)-2-oxa-6-thia-7-aza-3(6,1)-diazepana-1(4,2)-pyrimidina-5(1,3)-benzenacycloheptaphan-4-one 6,6-dioxide (2.6 mg, 11%) ESI-MS m/z calc. 570.2049, found 571.0 (M+1)⁺; Retention time: 0.97 minutes (LC method A).

### Example 21: Preparation of Compound 67 and Compound 68

### Step 1: (16R)-18-Benzyl-12-(2,6-dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 67)

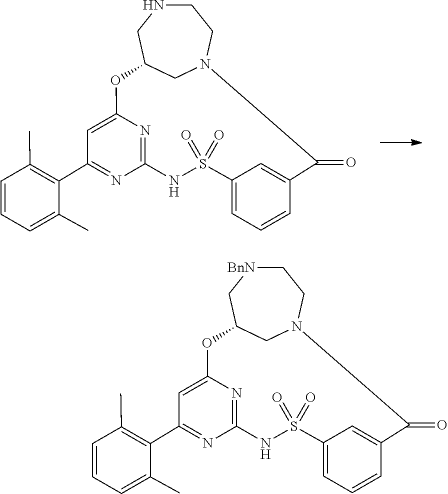

A 4 mL vial was charged with (16R)-12-(2,6-dimethylphenyl)-15-oxa-8λ⁶-thia-1,9,11,18,22-pentaazatetracyclo[14.4.1.13,7.110,14]tricosa-3,5,7(23),10,12,14(22)-hexaene-2,8,8-trione (hydrochloride salt) (25 mg, 0.04845 mmol), anhydrous DCM (500 μL), DIEA (15 μL, 0.08612 mmol) (dissolved all solid), benzaldehyde (10 μL, 0.09838 mmol) and acetic acid (10 μL, 0.1758 mmol). The vial was briefly purged with nitrogen, capped and stirred at room temperature for about 20 minutes. Sodium triacetoxyborohydride (20 mg, 0.09437 mmol) was added. The vial was purged with nitrogen, capped and the reaction was stirred at room temperature for one hour. A bit of methanol was added. The DCM was evaporated, and the residue was taken in DMSO (1 mL). The solution was microfiltered and purified by reverse phase preparative HPLC using a gradient of acetonitrile in water (1 to 99% over 15 min) and HCl as a modifier to give (16R)-18-benzyl-12-(2,6-dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (17 mg, 57%) as a white solid. ESI-MS m/z calc. 569.20966, found 570.44 (M+1)⁺; Retention time: 1.07 minutes (LC method A). ¹H NMR (400 MHz, DMSO-d₆+10% D₂O (broad signals in the absence of D₂O) δ 8.67 (s, 1H), 7.96 (d, J=7.8 Hz, 1H), 7.76-7.65 (m, 2H), 7.67-7.51 (m, 5H), 7.31 (t, J=7.9 Hz, 1H), 7.16 (d, J=7.6 Hz, 2H), 6.28 (s, 1H), 5.75 (broad s, 1H), 4.66-4.35 (m, 3H), 3.86-3.51 (m, 5H), 3.51-3.40 (m, 1H), 3.38-3.24 (m, 1H), 2.06 (br s, 6H).

### Step 2: (16R)-18-Benzyl-12-(2,6-dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 67), and (16S)-18-benzyl-12-(2,6-dimethylphenyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione (Compound 68)

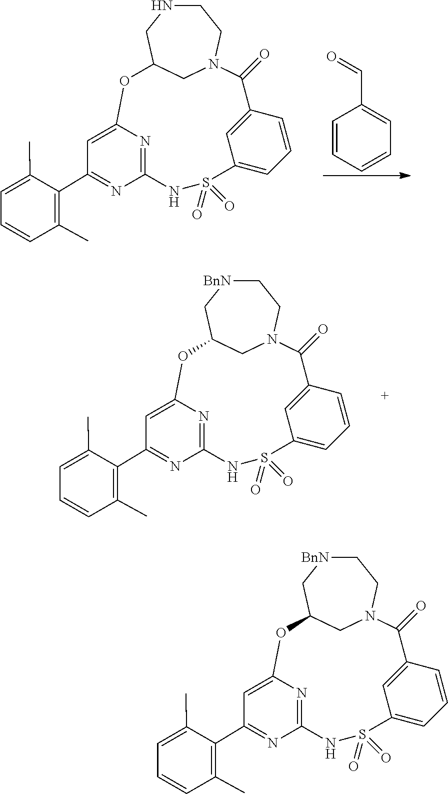

12-(2,6-Dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (25 mg, 0.05208 mmol) and benzaldehyde (25 μL, 0.2459 mmol) were combined in dichloroethane (0.5 mL) with acetic acid (15 μL, 0.2638 mmol). After 45 minutes, sodium triacetoxyborohydride (45 mg, 0.2123 mmol) was added and the reaction was stirred at room temperature for 2 hours. At this point conversion appeared to have stalled, and additional benzaldehyde (25 μL, 0.2459 mmol) and acetic acid (15 μL, 0.2638 mmol) was added. After the reaction was stirred for an additional hour, sodium cyanoborohydride (16 mg, 0.2546 mmol) was added and the reaction was stirred for an additional 16 hours at room temperature. After this time the reaction mixture was diluted with methanol, filtered, and purified by reverse phase HPLC (1-70% ACN in water, HCl modifier, 15 min run), to give 18-benzyl-12-(2,6-dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (22 mg, 70%) ESI-MS m/z calc. 569.20966, found 570.4 (M+1)⁺; Retention time: 0.48 minutes (LC method D).

This material was then submitted for chiral SFC separation (ChiralCel OJ-H (250×10 mm, 5 m column, mobile phase: 28% MeCN/MeOH (90:10, 20 mM NH₃, 72% CO₂, concentration 14 mg/mL in MeCN/MeOH/DMSO (81:9:10), injection volume 70 μL, 100 bar, 220 nm) to give each enantiomer as a white solid. First to elute, peak 1, (16R)-18-benzyl-12-(2,6-dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (4 mg, 13%) ESI-MS m/z calc. 569.20966, found 570.5 (M+1)⁺; Retention time: 1.16 minutes (LC method A), and second to elute, peak 2, (16S)-18-benzyl-12-(2,6-dimethylphenyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione (4.4 mg, 15%). ESI-MS m/z calc. 569.20966, found 570.5 (M+1)⁺; Retention time: 1.16 minutes (LC method A).

### Example 22: Preparation of Compound 69

### Step 1: 18-Benzyl-12-(2,6-dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione, enantiomer 1, and 18-benzyl-12-(2,6-dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione, Enantiomer 2

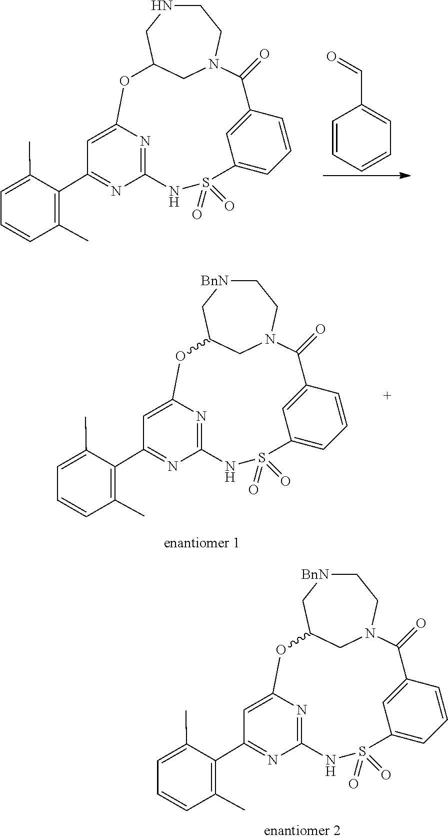

12-(2,6-Dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (660 mg, 1.375 mmol) and benzaldehyde (approximately 689.0 mg, 660.0 μL, 6.493 mmol) were combined in dichloroethane (13.20 mL) with acetic acid (approximately 418.2 mg, 396.0 μL, 6.964 mmol). After 45 minutes, sodium cyanoborohydride (approximately 422.4 mg, 6.722 mmol) was added and the reaction was stirred at room temperature for 2 hours at room temperature. After this time the reaction mixture was diluted with methanol, filtered, and purified by preparative HPLC (1-70% ACN in water, HCl modifier, 15 min run), to give 18-benzyl-12-(2,6-dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (750 mg, 96%) ESI-MS m/z calc. 569.20966, found 570.5 (M+1)⁺; Retention time: 1.14 minutes, (LC method A). This material was then submitted for chiral SFC separation (ChiralCel OJ-H (250×21.2 mm, 5 m column, mobile phase: MeCN/MeOH (90:10, 20 mM NH₃, 72% CO₂, flow 70 mL/min, 24 mg/mL in MeCN/MeOH/DMSO (81/9/10), injection volume 500 μL, 100 bar, 220 nm) to give, first to elute as Peak 1, 18-benzyl-12-(2,6-dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (121 mg, 31%) as a white solid, ESI-MS m/z calc. 569.20966, found 570.5 (M+1)⁺; Retention time: 1.14 minutes; LC method A. The second isomer was discarded.

### Step 2: 12-(2,6-Dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione

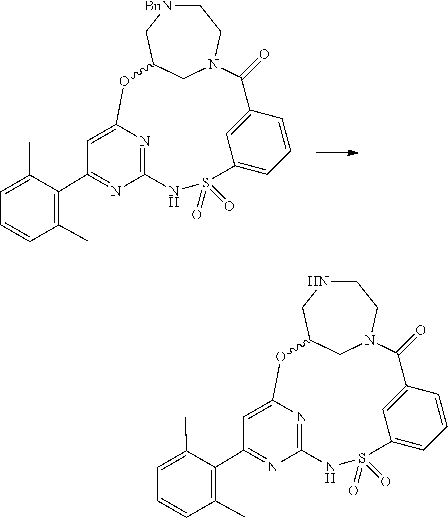 [SMILES: Cc1cccc(C)c1-c1cc2nc(n1)NS(=O)(=O)c1cccc(c1)C(=O)N1CCN(Cc3ccccc3)CC(C1)O2 ; Cc1cccc(C)c1-c1cc2nc(n1)NS(=O)(=O)c1cccc(c1)C(=O)N1CCNCC(C1)O2]

18-Benzyl-12-(2,6-dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (121 mg, 0.2124 mmol) (Peak 1 from step 1 separation) was dissolved in methanol (22 mL) in a nitrogen-purged flask, with brief sonication to aid in dissolving the starting material. Dihydroxypalladium (45 mg, 0.06409 mmol) was added, and the reaction mixture was then purged with hydrogen gas by bubbling through the reaction from a balloon for 15 minutes, then stirred for 3 hours under hydrogen. The reaction vessel was then purged with nitrogen, and the reaction mixture was filtered through Celite, which was washed with 100 mL methanol. The filtrate was concentrated to give a white solid, 12-(2,6-dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (97 mg, 95%) ESI-MS m/z calc. 479.16272, found 480.3 (M+1)⁺; Retention time: 0.79 minutes; LC method A.

### Step 3: 12-(2,6-Dimethylphenyl)-18-{2-[1-(trifluoromethyl)cyclopropyl]ethyl}-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 69)

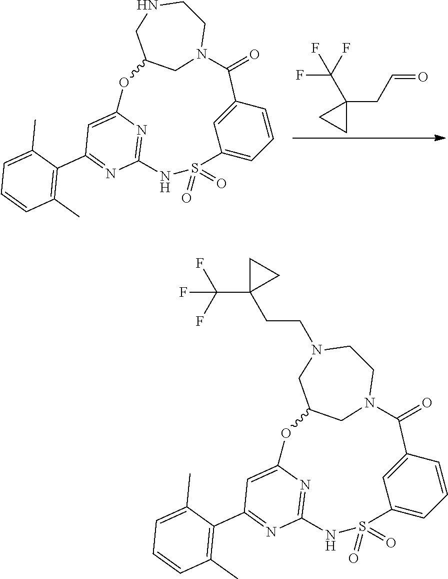 [SMILES: Cc1cccc(C)c1-c1cc2nc(n1)NS(=O)(=O)c1cccc(c1)C(=O)N1CCNCC(C1)O2 ; O=CCC1(C(F)(F)F)CC1 ; Cc1cccc(C)c1-c1cc2nc(n1)NS(=O)(=O)c1cccc(c1)C(=O)N1CCN(CCC3(C(F)(F)F)CC3)CC(C1)O2]

To a vial containing 12-(2,6-dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (28 mg, 0.05839 mmol) was added dichloroethane (1 mL), 2-[1-(trifluoromethyl)cyclopropyl]acetaldehyde (44 mg, 0.2893 mmol), and acetic acid (17 μL, 0.2989 mmol). The reaction was stirred at rt for 1 h. Sodium cyanoborohydride (19 mg, 0.3023 mmol) was added and the reaction was allowed to stir at rt for 1 h. The reaction was quenched with methanol, filtered, and purified by preparative HPLC (1%-99% ACN:water with a 0.1% HCl modifier) to provide 12-(2,6-dimethylphenyl)-18-{2-[1-(trifluoromethyl)cyclopropyl]ethyl}-15-oxa-8λ⁶-thia-1,9,11,18,22-pentaazatetracyclo[14.4.1.13,7.110,14]tricosa-3(23),4,6,10(22),11,13-hexaene-2,8,8-trione (17.4 mg, 48%) as a white solid. ESI-MS m/z calc. 615.2127, found 616.3 (M+1)⁺; Retention time: 1.18 minutes; LC method A.

### Example 23: Preparation of Compound 70

### Step 1: Methyl 6-benzylsulfanylpyridine-2-carboxylate

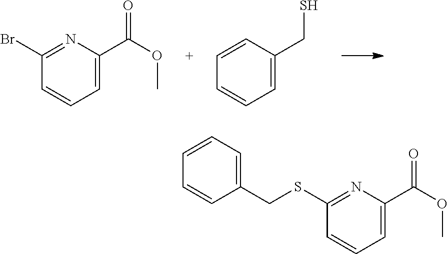

To a solution of phenylmethanethiol (28.408 g, 26.800 mL, 228.72 mmol) in THE (600 mL) was added NaH (11.200 g, 60% w/w, 280.03 mmol) in a few portions at 0° C. The slurry was warmed to room temperature and stirred for 30 min, then methyl 6-bromopyridine-2-carboxylate (50 g, 231.45 mmol) was added as a single portion. After 3 h, the reaction was diluted with ether (800 mL) and quenched with water (400 mL) and saturated sodium bicarbonate (50 mL). The layers were separated, and the organic layer was washed with brine, dried over sodium sulfate, and concentrated under reduced pressure to yield methyl 6-benzylsulfanylpyridine-2-carboxylate (56.35 g, 89%) as a yellow oil. ¹H NMR (500 MHz, DMSO-d₆) δ 7.84-7.77 (m, 1H), 7.77-7.73 (m, 1H), 7.52 (m, 1H), 7.48 (d, J=7.8 Hz, 2H), 7.28 (t, J=7.2, 7.2 Hz, 2H), 7.24-7.18 (m, 1H), 4.44 (s, 2H), 3.90 (d, J=1.2 Hz, 3H). ESI-MS m/z calc. 259.0667, found 260.1 (M+1)⁺; Retention time: 3.2 minutes; LC method T.

### Step 2: Methyl 6-chlorosulfonylpyridine-2-carboxylate

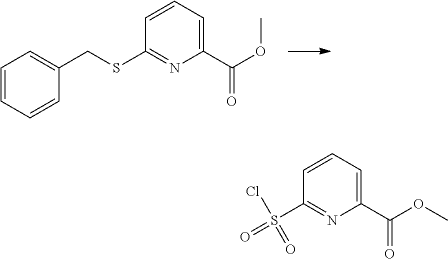 [SMILES: COC(=O)c1cccc(SCc2ccccc2)n1 ; COC(=O)c1cccc(S(=O)(=O)Cl)n1]

A solution of methyl 6-benzylsulfanylpyridine-2-carboxylate (121.62 g, 431.47 mmol) in DCM (950 mL) and DI water (300 mL) was cooled in a −1-0° C. ice bath and, with vigorous stirring, sulfuryl chloride (228.14 g, 140 mL, 1.6396 mol) was added dropwise while the temperature was maintained below 5° C. After the addition, the organic phase was separated, washed with DI water (2×500 mL), dried over anhydrous sodium sulfate, filtered and concentrated under vacuum. The residue was dissolved in DCM (500 mL). Hexanes (1000 mL) was added and the DCM was slowly evaporated off. The white precipitate was filtered by vacuum and the solids were washed with Hexanes (2×500 mL). The filtered solids were collected. The residue solids in the filtrate were filtered and dissolved in DCM (500 mL). The DCM solution was transferred to a 1 L round-bottom flask and concentrated under vacuum. The residue was dissolved in DCM (200 mL). Hexanes (600 mL) was added and the DCM was slowly evaporated off. The white precipitation was filtered by vacuum and the solids were washed with hexanes (2×500 mL) After drying, methyl 6-chlorosulfonylpyridine-2-carboxylate (56.898 g, 55%) was isolated. ¹H NMR (500 MHz, Chloroform-d) δ 8.48 (dd, J=7.8, 1.1 Hz, 1H), 8.31 (dd, J=7.9, 1.1 Hz, 1H), 8.25 (t, J=7.8 Hz, 1H), 4.08 (s, 3H). ESI-MS m/z calc. 234.97061, found 236.1 (M+1)⁺; Retention time: 1.74 minutes; LC method T.

### Step 3: Methyl 6-[[4-chloro-6-(2,6-dimethylphenyl)pyrimidin-2-yl]sulfamoyl]pyridine-2-carboxylate

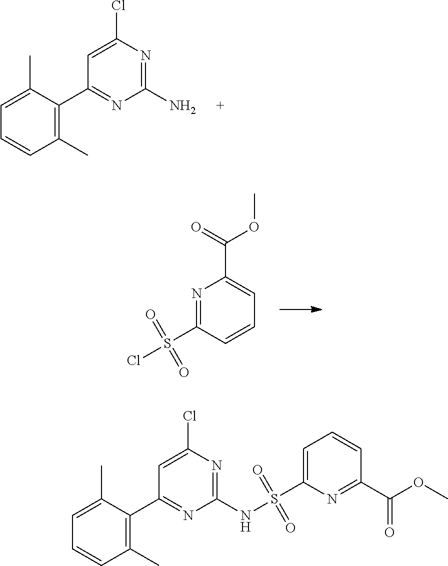

A solution of 4-chloro-6-(2,6-dimethylphenyl)pyrimidin-2-amine (16.63 g, 71.161 mmol) and methyl 6-chlorosulfonylpyridine-2-carboxylate (16.8 g, 71.294 mmol) dissolved in anhydrous THE (680 mL) was cooled to −78° C. Then Lithium bis(trimethylsilyl)amide (143 mL of 1 M, 143.00 mmol) in solution in THE was added dropwise. The mixture was allowed to warm up to 0° C. slowly and then 1M aqueous HCl (146 mL) was added, followed by DI water (680 mL). The THF was evaporated and the aqueous phase was extracted with chloroform (3×250 mL). The combined organic layers were washed with saturated aqueous NaCl (300 mL), dried over anhydrous sodium sulfate, filtered, and concentrated under vacuum. The crude was recrystallized in 10% Acetone in Hexanes (500 mL). The white precipitate was filtered and rinsed with acetone (2×100 mL) to give methyl 6-[[4-chloro-6-(2,6-dimethylphenyl)pyrimidin-2-yl]sulfamoyl]pyridine-2-carboxylate (15.79 g, 50%). ESI-MS m/z calc. 432.06592, found 433.3 (M+1)⁺; Retention time: 5.5 minutes; LC method S.

### Step 4: 6-[[4-Chloro-6-(2,6-dimethylphenyl)pyrimidin-2-yl]sulfamoyl]pyridine-2-carboxylic acid

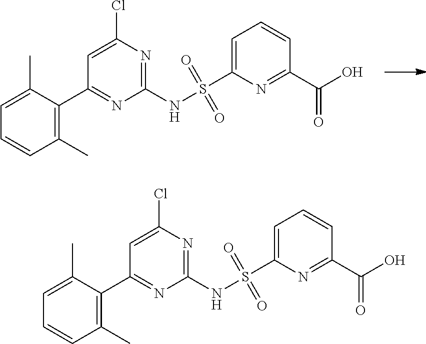 [SMILES: Cc1cccc(C)c1-c1cc(Cl)nc(NS(=O)(=O)c2cccc(C(=O)O)n2)n1 ; Cc1cccc(C)c1-c1cc(Cl)nc(NS(=O)(=O)c2cccc(C(=O)O)n2)n1]

To a solution of methyl 6-[[4-chloro-6-(2,6-dimethylphenyl)pyrimidin-2-yl]sulfamoyl]pyridine-2-carboxylate (15.79 g, 36.477 mmol) in THE (180 mL) was added aqueous sodium hydroxide (182 mL of 1 M, 182.00 mmol). The reaction was stirred at RT for 1 h. The THF was evaporated, and the aqueous layer was washed with diethyl ether (2×200 mL). The aqueous layer was acidified to pH 2 with 1 M Aqueous HCl (250 mL). The precipitate was filtered and the a white solid were rinsed with DI water (2×250 mL). The solids were dried under vacuum to give 6-[[4-chloro-6-(2,6-dimethylphenyl)pyrimidin-2-yl]sulfamoyl]pyridine-2-carboxylic acid (14.3444 g, 93%). ¹H NMR (250 MHz, DMSO-d₆) δ 8.14-7.99 (m, 3H), 7.21-7.11 (m, 1H), 7.03 (d, J=7.7 Hz, 2H), 6.92 (s, 1H), 1.78 (s, 6H). ESI-MS m/z calc. 418.05026, found 419.1 (M+1)⁺; Retention time: 2.61 minutes; LC method T.

### Step 5: 1,4-Diazepan-6-ol

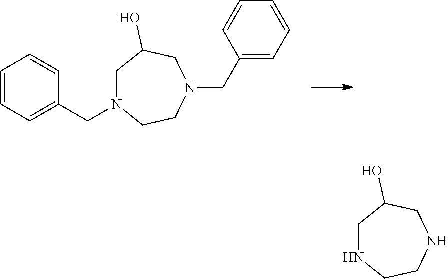 [SMILES: OC1CN(Cc2ccccc2)CCN(Cc2ccccc2)C1 ; OC1CNCCNC1]

Pd(OH)₂ on Carbon (6 g, 8.5 mmol, 20 wt %) was added to a solution of 1,4-dibenzyl-1,4-diazepan-6-ol (54 g, 182 mmol) in MeOH (1400 mL). The mixture was hydrogenated for 16 hours under hydrogen atmosphere. The reaction was filtered over Celite and concentrated to give 1,4-diazepan-6-ol (20.6 g, 92%) as a colorless oil. ESI-MS m/z calc. 116.09496, found 117.3 (M+1)⁺; Retention time: 0.78 minutes; LC method T.

### Step 6: Benzyl 6-hydroxy-1,4-diazepane-1-carboxylate

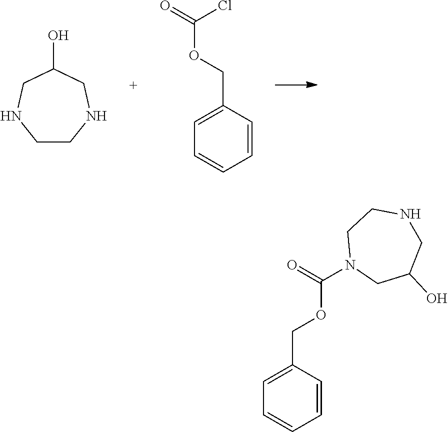 [SMILES: OC1CNCCNC1 ; O=C(Cl)OCc1ccccc1 ; O=C(OCc1ccccc1)N1CCNCC(O)C1]

To a solution of 1,4-diazepan-6-ol (7.17 g, 58.6 mmol) in MeOH (100 ML) was added ethyl trifluoroacetate (7.2 mL, 59.9 mmol) slowly at OC. The solution was stirred at room temperature for 1 hour. Then, the reaction was cooled down to OC, TEA (10.0 mL, 69.6 mmol) and benzyl chloroformate (22 mL of 2.7 M, 59.4 mmol) were added slowly. The reaction was stirred at room temperature for 1 hour. Potassium carbonate (13 g, 94.1 mmol) in water (5 mL) was added. The reaction was stirred at 40° C. for 14 hours. After filtration, the solvent was removed under reduced pressure. The residue was purified by silica gel chromatography using a gradient of MeOH/ethyl acetate 0-60% to give benzyl 6-hydroxy-1,4-diazepane-1-carboxylate (3.6 g, 23%) as a colorless oil. H NMR (250 MHz, CD₃OD) δ 7.66-7.08 (m, 5H), 5.14 (s, 2H), 4.14-3.87 (m, 1H), 3.84-3.61 (m, 2H), 3.59-3.36 (m, 2H), 3.16-2.76 (m, 4H). ESI-MS m/z calc. 250.13174, found 251.3 (M+1)⁺; Retention time: 1.81 minutes; LC method T.

### Step 7: Benzyl 4-(3,3-dimethylbutyl)-6-hydroxy-1,4-diazepane-1-carboxylate

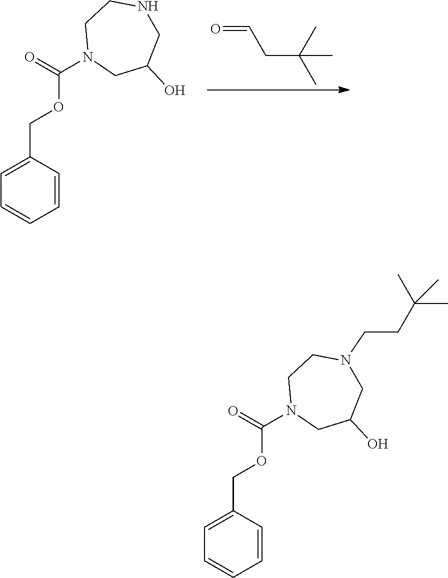

Benzyl 6-hydroxy-1,4-diazepane-1-carboxylate (608.5 mg, 2.431 mmol) in DCE (5 mL) was combined with 3,3-dimethylbutanal (460 μL, 3.665 mmol), acetic acid (500 μL, 8.792 mmol), and stirred for 1 hour at room temperature. Sodium cyanoborohydride (760 mg, 12.09 mmol) was then added and the reaction was stirred for 90 min. at room temperature. The crude was filtered and purified by reverse phase preparative chromatography using a C₁₈ column and a 15 min. gradient eluent of 1 to 50% acetonitrile in water containing 5 mM hydrochloric acid to give benzyl 4-(3,3-dimethylbutyl)-6-hydroxy-1,4-diazepane-1-carboxylate (547.6 mg, 67%). ESI-MS m/z calc. 334.22565, found 335.0 (M+1)⁺; Retention time: 0.98 minutes; LC method A.

### Step 8: 18-(3,3-Dimethylbutyl)-12-(2,6-dimethylphenyl)-15-oxa-8λ⁶-thia-1,9,11,18,22,23-hexaazatetracyclo[14.4.1.13,7.110,14]tricosa-3,5,7(23),10,12,14(22)-hexaene-2,8,8-trione (Compound 70)

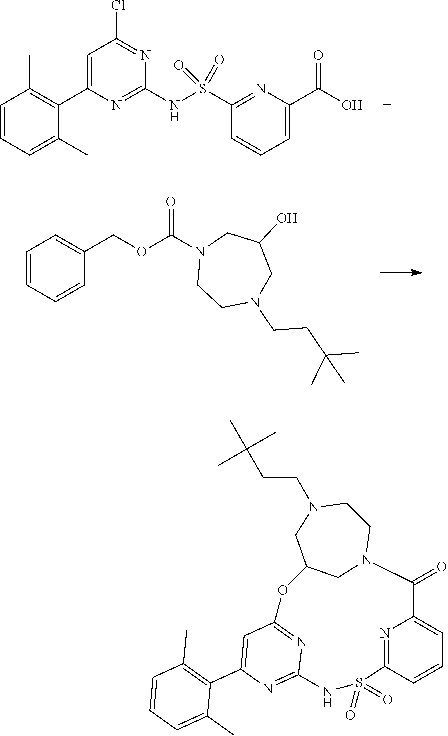 [SMILES: Cc1cccc(C)c1-c1cc(Cl)nc(NS(=O)(=O)c2cccc(C(=O)O)n2)n1 ; CC(C)(C)CCN1CCN(C(=O)OCc2ccccc2)CC(O)C1 ; Cc1cccc(C)c1-c1cc2nc(n1)NS(=O)(=O)c1cccc(n1)C(=O)N1CCN(CCC(C)(C)C)CC(C1)O2]

Stage 1: 6-[[4-Chloro-6-(2,6-dimethylphenyl)pyrimidin-2-yl]sulfamoyl]pyridine-2-carboxylic acid (170 mg, 0.4059 mmol) and benzyl 4-(3,3-dimethylbutyl)-6-hydroxy-1,4-diazepane-1-carboxylate (135 mg, 0.4036 mmol) were combined and dissolved in tetrahydrofuran (1.5 mL). Sodium tert-butoxide (97 mg, 1.009 mmol) was added. The reaction mixture was allowed to stir at 50° C. for 3 hours. More sodium tert-butoxide (97 mg, 1.009 mmol) and tetrahydrofuran (1.5 mL) were added and the reaction was continued at room temperature for 18 h. The reaction mixture was cooled down to room temperature, filtered, and purified by reverse phase preparative chromatography using a C₁₈ column and a 15 min. gradient eluent of 10 to 60% acetonitrile in water containing 5 mM hydrochloric acid to give 6-[[4-[[1-benzyloxycarbonyl-4-(3,3-dimethylbutyl)-1,4-diazepan-6-yl]oxy]-6-(2,6-dimethylphenyl)pyrimidin-2-yl]sulfamoyl]pyridine-2-carboxylic acid (29.8 mg, 10%) ESI-MS m/z calc. 716.2992, found 716.0 (M+1)⁺; Retention time: 1.3 minutes (LC method A), and 6-[[4-[[1-(3,3-dimethylbutyl)-1,4-diazepan-6-yl]oxy]-6-(2,6-dimethylphenyl)pyrimidin-2-yl]sulfamoyl]pyridine-2-carboxylic acid (45.5 mg, 18%). ¹H NMR (400 MHz, DMSO-d₆) δ 8.31-8.20 (m, 2H), 8.18-8.11 (m, 1H), 7.29 (t, J=7.6 Hz, 1H), 7.16 (d, J=7.6 Hz, 2H), 6.30 (d, J=20.3 Hz, 1H), 3.85-3.74 (m, 2H), 3.57 (ddd, J=21.9, 10.1, 6.1 Hz, 2H), 3.46-3.23 (m, 3H), 3.22-2.80 (m, 4H), 2.24-2.08 (m, 6H), 1.73-1.43 (m, 2H), 0.91 (d, J=16.9 Hz, 9H). ESI-MS m/z calc. 582.26245, found 583.0 (M+1)⁺; Retention time: 0.92 minutes (LC method A).

Stage 2: 6-[[4-[[1-(3,3-Dimethylbutyl)-1,4-diazepan-6-yl]oxy]-6-(2,6-dimethylphenyl)pyrimidin-2-yl]sulfamoyl]pyridine-2-carboxylic acid (45.5 mg, 18%), HATU (100 mg, 0.2630 mmol), DIEA (300 μL, 1.722 mmol) and DMF (1 mL) were stirred at room temperature for 30 min. The crude was filtered and purified by reverse phase preparative chromatography using a C₁₈ column and a 15 min gradient eluent of 1 to 50% acetonitrile in water containing 5 mM hydrochloric acid to give 18-(3,3-dimethylbutyl)-12-(2,6-dimethylphenyl)-15-oxa-8λ⁶-thia-1,9,11,18,22,23-hexaazatetracyclo[14.4.1.13,7.110,14]tricosa-3,5,7(23),10,12,14(22)-hexaene-2,8,8-trione (1.8 mg, 1%). ESI-MS m/z calc. 564.2519, found 565.0 (M+1)⁺; Retention time: 1.03 minutes (LC method A).

### Example 24: Preparation of Compound 71

### Step 1: 2-[(2R)-3-(tert-Butylamino)-2-hydroxy-propyl]isoindoline-1,3-dione

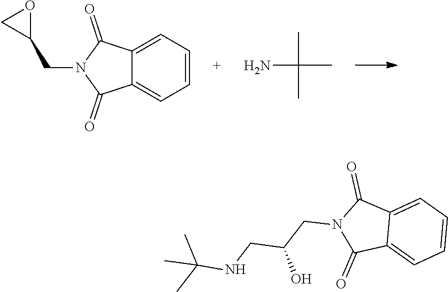

A pressure vessel was charged with a solution of 2-methylpropan-2-amine (2.16 g, 29.534 mmol) and 2-[[(2S)-oxiran-2-yl]methyl]isoindoline-1,3-dione (5 g, 24.607 mmol) in isopropanol (160 mL). The reaction mixture was stirred at 85° C. overnight. Isopropanol was evaporated under reduced pressure. The residue was purified by silica gel chromatography (DCM/MeOH=100/0-90/10) to afford 2-[(2R)-3-(tert-butylamino)-2-hydroxy-propyl]isoindoline-1,3-dione (5.65 g, 78%) as white solid. ESI-MS m/z calc. 276.1474, found 277.2 (M+1)⁺; Retention time: 1.78 minutes; LC method T.

### Step 2: 2-[(2R)-3-(tert-Butylamino)-2-[tert-butyl(dimethyl)silyl]oxy-propyl]isoindoline-1,3-dione

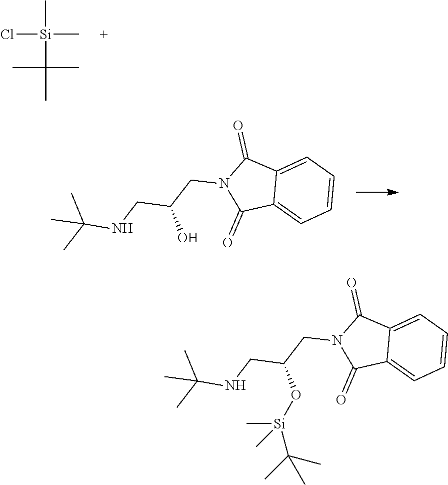

To a solution of 2-[(2R)-3-(tert-butylamino)-2-hydroxy-propyl]isoindoline-1,3-dione (4.8 g, 16.328 mmol) and imidazole (2.223 g, 32.654 mmol) in DMF (60 mL) was added tert-butyl-chloro-dimethyl-silane (4.925 g, 32.676 mmol). The reaction mixture was stirred at room temperature for 36 hours. Imidazole (0.74 g, 10.87 mmol) and tert-butyl-chloro-dimethyl-silane (1.64 g, 10.87 mmol) were added to the reaction mixture, and it was stirred for 60 hours. The reaction mixture was quenched with brine (150 mL), and the water layer was extracted with ethyl acetate (3×150 mL). The combined organic layers were washed with brine (3×150 mL), dried over anhydrous sodium sulfate and concentrated under vacuum. The residue was purified by silica gel chromatography using 0 to 10% MeOH in DCM to afford 2-[(2R)-3-(tert-butylamino)-2-[tert-butyl(dimethyl)silyl]oxy-propyl]isoindoline-1,3-dione (4.47 g, 68%) as a light yellow liquid. ESI-MS m/z calc. 390.23386, found 391.7 (M+1)⁺; Retention time: 3.25 minutes; LC method T.

### Step 3: Ethyl 2-[tert-butyl-[(2R)-2-[tert-butyl(dimethyl)silyl]oxy-3-(1,3-dioxoisoindolin-2-yl)propyl]amino]acetate

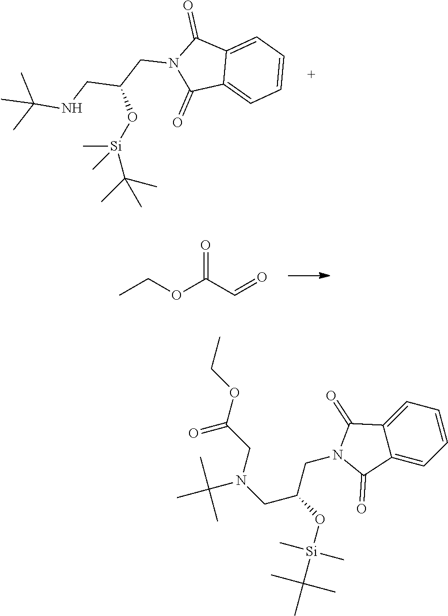

To a solution of 2-[(2R)-3-(tert-butylamino)-2-(1-methyl-1-trimethylsilyl-ethoxy)propyl]isoindoline-1,3-dione (4.85 g, 12.045 mmol) in DCM (150 mL) were added ethyl 2-oxoacetate (2.459 g, 50% w/w, 12.043 mmol) followed by sodium triacetoxyborohydride (2.553 g, 12.046 mmol). The reaction mixture was stirred at room temperature. Ethyl 2-oxoacetate (2.459 g, 50% w/w, 12.043 mmol) and sodium triacetoxyborohydride (2.553 g, 12.046 mmol) were added to the reaction mixture every 2 hours for a total of 10 times over 2 days. The reaction mixture was quenched with saturated sodium bicarbonate (150 mL), and stirred for 0.5 hour. Two layers were separated, and the aqueous layer was extracted with dichloromethane (3×120 mL). The combined dichloromethane layers were washed with brine (250 mL), dried over anhydrous sodium sulfate, concentrated under vacuum. The residue was purified by silica gel chromatography (Hexane/EtOAc=100/0-60/40) to afford ethyl 2-[tert-butyl-[(2R)-3-(1,3-dioxoisoindolin-2-yl)-2-(1-methyl-1-trimethylsilyl-ethoxy)propyl]amino]acetate (5.22 g, 91%) as a clear oil. ESI-MS m/z calc. 476.27066, found 477.5 (M+1)⁺; Retention time: 3.61 minutes; LC method T.

### Step 4: (6S)-4-tert-Butyl-6-[tert-butyl(dimethyl)silyl]oxy-1,4-diazepan-2-one

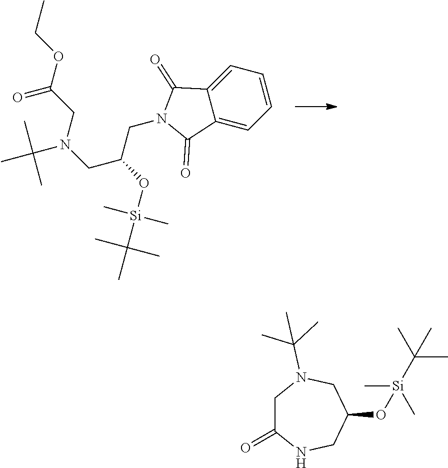 [SMILES: CCOC(=O)CN(C[C@H](CN1C(=O)c2ccccc2C1=O)O[Si](C)(C)C(C)(C)C)C(C)(C)C ; CC(C)(C)N1CC(=O)NC[C@H](O[Si](C)(C)C(C)(C)C)C1]

To a solution of ethyl 2-[tert-butyl-[(2R)-2-[tert-butyl(dimethyl)silyl]oxy-3-(1,3-dioxoisoindolin-2-yl)propyl]amino]acetate (5.22 g, 11.283 mmol) in ethanol (150 mL) was added hydrazine hydrate (2.8244 g, 3.76 mL, 41.186 mmol). The reaction mixture was stirred at 85° C. for 18 hours. After cooling to room temperature, the solvent was removed under reduced pressure. The residue was diluted with 10% NaOH (aqueous) (100 mL), and extracted with ethyl acetate (3×100 mL). The combined organic layers were washed with brine (100 mL), dried over anhydrous sodium sulfate and concentrated under vacuum. The residue was purified by silica gel chromatography using 0 to 40% acetone in hexanes to afford (6S)-4-tert-butyl-6-[tert-butyl(dimethyl)silyl]oxy-1,4-diazepan-2-one (2.6 g, 77%) as a light yellow solid. ESI-MS m/z calc. 300.2233, found 301.5 (M+1)⁺; Retention time: 2.67 minutes; LC method T.

### Step 5: tert-Butyl (6R)-4-tert-butyl-6-hydroxy-1,4-diazepane-1-carboxylate

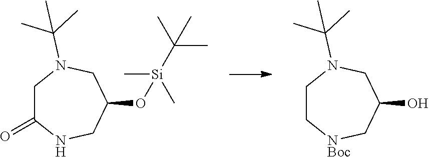 [SMILES: CC(C)(C)N1CC(=O)NC[C@H](O[Si](C)(C)C(C)(C)C)C1 ; CC(C)(C)OC(=O)N1CCN(C(C)(C)C)C[C@@H](O)C1]

Stage 1: To a solution of (6S)-4-tert-butyl-6-[tert-butyl(dimethyl)silyl]oxy-1,4-diazepan-2-one (2.6 g, 8.6519 mmol) in anhydrous THE (55 mL) was added LAH (1.97 g, 51.905 mmol) slowly at 0° C. The reaction mixture was stirred at 40° C. for 9 hours. The reaction was cooled to 0° C. in an ice batch, and it was diluted with diethyl ether (50 mL). The reaction was quenched with water (2.1 mL), 15% NaOH (2.1 mL) and water (6.3 mL), and it was stirred at room temperature for 30 minutes. The white precipitate was removed by filtration through a pad of Celite, and washed with THE (3×25 mL). The combined filtrate was concentrated under vacuum.

Stage 2: The residue was dissolved in THE (20 mL), and a aqueous solution of NaOH (17.3 g, 10% w/w, 43.253 mmol) was added, followed by Boc anhydride (1.98 g, 9.0723 mmol). The reaction was stirred at room temperature for 1 hour. Water (50 mL) and ethyl acetate (30 mL) were added. The organic layer was separated, and aqueous layer was extracted with ethyl acetate (2×30 mL). The combined organic layers were washed with brine (50 mL), dried over anhydrous sodium sulfate, filtered and concentrated under reduced pressure. The crude product was purified by silica gel chromatography using 0 to 50% ethyl acetate in hexane to afford tert-butyl (6R)-4-tert-butyl-6-hydroxy-1,4-diazepane-1-carboxylate (1.86 g, 78%) as a white solid. ¹H NMR (250 MHz, DMSO-d₆) δ 4.61 (t, J=5.4 Hz, 1H), 3.80-3.64 (m, 1H), 3.64-3.43 (m, 2H), 3.13-2.60 (m, 4H), 2.47-2.25 (m, 2H), 1.39 (s, 9H), 1.01 (s, 9H). ESI-MS m/z calc. 272.21, found 273.3 (M+1)⁺; Retention time: 1.13 minutes; LC method W.

### Step 6: (6S)-1-tert-Butyl-1,4-diazepan-6-ol

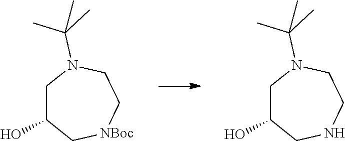 [SMILES: CC(C)(C)OC(=O)N1CCN(C(C)(C)C)C[C@@H](O)C1 ; CC(C)(C)N1CCNC[C@H](O)C1]

A 100 mL round bottom flask was charged with tert-butyl (6R)-4-tert-butyl-6-hydroxy-1,4-diazepane-1-carboxylate (1.04 g, 3.818 mmol) and dioxane (3 mL). After dissolution of the solids, HCl (12 mL of 4 M, 48.00 mmol) (4M dioxane solution) was added and the mixture was stirred at rt for 3 hours. The volatiles were removed under reduced pressure. The solid was treated with DCM/MeOH and hexanes and the solvents were evaporated. The operation was repeated 3 times. Drying in vacuo provided (6S)-1-tert-butyl-1,4-diazepan-6-ol (dihydrochloride salt) (1.018 g, 100%) as a white foamy solid. ESI-MS m/z calc. 172.15756, found 173.09 (M+1)⁺; Retention time: 0.15 minutes; LC method A.

### Step 7: 3-[(6R)-4-tert-Butyl-6-hydroxy-1,4-diazepane-1-carbonyl]-N-[4-chloro-6-(2,6-dimethylphenyl)pyrimidin-2-yl]benzenesulfonamide

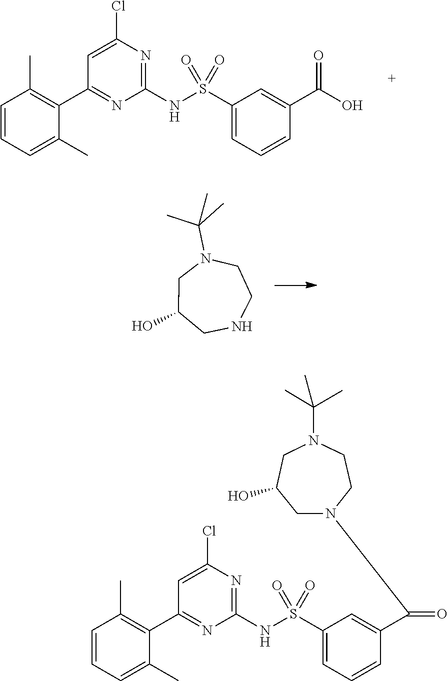 [SMILES: Cc1cccc(C)c1-c1cc(Cl)nc(NS(=O)(=O)c2cccc(C(=O)O)c2)n1 ; CC(C)(C)N1CCNC[C@H](O)C1 ; Cc1cccc(C)c1-c1cc(Cl)nc(NS(=O)(=O)c2cccc(C(=O)N3CCN(C(C)(C)C)C[C@@H](O)C3)c2)n1]

A 100 mL flask was charged under nitrogen with (6S)-1-tert-butyl-1,4-diazepan-6-ol (Dihydrochloride salt) (384 mg, 1.441 mmol), anhydrous DMF (6 mL) and 3-[[4-chloro-6-(2,6-dimethylphenyl)pyrimidin-2-yl]sulfamoyl]benzoic acid (504 mg, 1.206 mmol). After dissolution of the reagents, the mixture was cooled down in an ice bath. DIEA (1.4 mL, 8.038 mmol) and HATU (580 mg, 1.525 mmol) were added and the mixture was stirred at 0° C. for 19 minutes (complete after 10 min by LCMS). The reaction was quenched by being poured in citric acid (50 mL of 10% w/v, 26.02 mmol)(10% aqueous) under vigorous stirring and cooled in ice. The resulting white solid was filtered and washed with water. The wet solid was dissolved in DCM and the solution was dried over sodium sulfate. After evaporation of the solvents, the residue was dissolved in DCM and purified by flash chromatography on silica gel (80 g column) using a gradient of methanol (0 to 10% over 60 min) in dichloromethane. The product eluted around 5-6% methanol. Evaporation of the solvents gave 3-[(6R)-4-tert-butyl-6-hydroxy-1,4-diazepane-1-carbonyl]-N-[4-chloro-6-(2,6-dimethylphenyl)pyrimidin-2-yl]benzenesulfonamide (158 mg, 23%) as a white solid. ESI-MS m/z calc. 571.202, found 572.28 (M+1)⁺; Retention time: 1.27 minutes (LC method A).

### Step 8: (16R)-18-tert-Butyl-12-(2,6-dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 71)

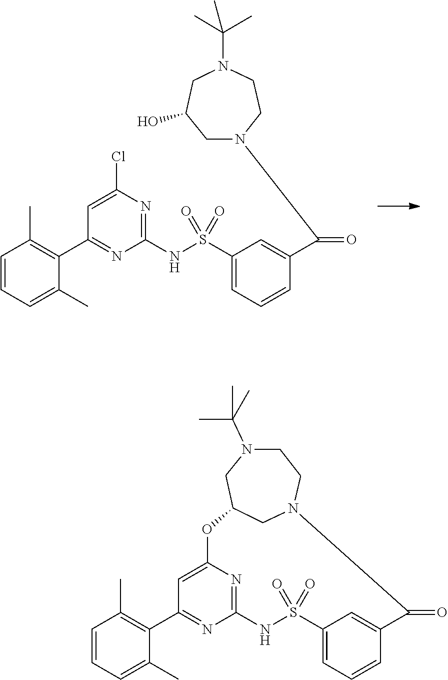 [SMILES: Cc1cccc(C)c1-c1cc(Cl)nc(NS(=O)(=O)c2cccc(C(=O)N3CCN(C(C)(C)C)C[C@@H](O)C3)c2)n1 ; Cc1cccc(C)c1-c1cc2nc(n1)NS(=O)(=O)c1cccc(c1)C(=O)N1CCN(C(C)(C)C)C[C@H](C1)O2]

A 100 mL flask was charged under nitrogen with 3-[(6R)-4-tert-butyl-6-hydroxy-1,4-diazepane-1-carbonyl]-N-[4-chloro-6-(2,6-dimethylphenyl)pyrimidin-2-yl]benzenesulfonamide (155 mg, 0.2709 mmol) and anhydrous DMF (8 mL). The mixture was cooled down in ice. NaH (93 mg of 60% w/w, 2.325 mmol) (60% mineral oil dispersion) was added in one portion. The mixture was stirred under nitrogen at 0° C. for 10 minutes. The ice bath was removed, and the reaction was vigorously stirred under nitrogen for 4 hours. The reaction mixture was slowly poured into an ice-cold citric acid (40 mL of 10% w/v, 20.82 mmol) aqueous solution under stirring. The resulting solid suspension was extracted with EtOAc (3×40 mL). A significant amount of product was detected in the aqueous phase (pH=2). The aqueous phase was neutralized to pH=6 using aqueous saturated sodium carbonate and the rest of the product was extracted with EtOAc (50 mL). The combined extracts were dried over sodium sulfate and the solvent was evaporated. After evaporation of the solvents, the residue was dissolved in DMSO (4 mL). The solution was purified by reverse phase preparative HPLC (Cis) using a gradient of acetonitrile in water (1 to 99% over 15 min) and HCl as a modifier to give 89 mg of product that contained large amounts of impurities. The product was dissolved in DMSO (2 mL) and purified a second time using a 21.2×50 mm C₁₈ column and a shallower gradient (1 to 50% over 25 min) of acetonitrile in water (HCl as a modifier). Evaporation and trituration in DCM/MeOH/hexanes provided after evaporation (16R)-18-tert-butyl-12-(2,6-dimethylphenyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (58 mg, 37%) as a white solid. ¹H NMR (499 MHz, DMSO-d₆+10% D₂O) δ 8.69 (s, 1H), 7.97-7.89 (m, 1H), 7.72-7.61 (m, 2H), 7.25 (t, J=7.6 Hz, 1H), 7.11 (d, J=7.7 Hz, 2H), 6.23 (s, 1H), 5.70 (broad s, 1H), 4.41-4.26 (m, 1H), 3.88 (d, J=12.8 Hz, 1H), 3.80 (d, J=13.6 Hz, 1H), 3.76-3.69 (m, 1H), 3.62 (t, J=12.0 Hz, 1H), 3.56-3.42 (m, 2H), 3.28 (dd, J=14.5, 10.8 Hz, 1H), 2.02 (br s, 6H), 1.43 (s, 9H). ESI-MS m/z calc. 535.22534, found 536.6 (M+1)⁺; Retention time: 0.9 minutes; LC method A.

### Example 25: Preparation of Compound 72 and Compound 73

### Step 1: 2-[(2R)-2-Hydroxy-3-(1,2,2-trimethylpropylamino)propyl]isoindoline-1,3-dione

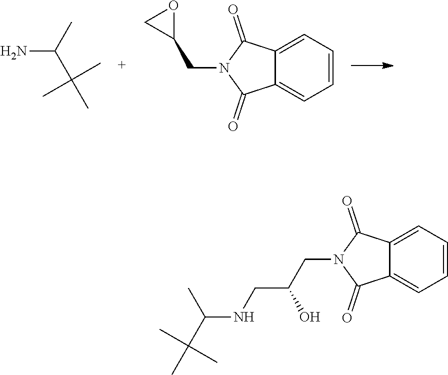

Into a round bottom flask was charged with a solution of 3,3-dimethylbutan-2-amine (6.985 g, 69.029 mmol) and 2-[[(2S)-oxiran-2-yl]methyl]isoindoline-1,3-dione (11.689 g, 57.524 mmol) in isopropanol (150 mL). The reaction mixture was refluxed overnight. Isopropanol was removed under vacuum. The residue was purified by silica gel chromatography using 0 to 10% methanol in dichloromethane (buffered with 1% ammonium hydroxide) to furnish 2-[(2R)-2-hydroxy-3-(1,2,2-trimethylpropylamino)propyl]isoindoline-1,3-dione (15.588 g, 89%) as a yellow oil, which solidified upon standing. The product is a mixture of diastereomers. ESI-MS m/z calc. 304.17868, found 305.2 (M+1)⁺; Retention time: 2.47 minutes; LC method S.

### Step 2: 2-[(2R)-2-[tert-Butyl(dimethyl)silyl]oxy-3-(1,2,2-trimethylpropylamino)propyl]isoindoline-1,3-dione

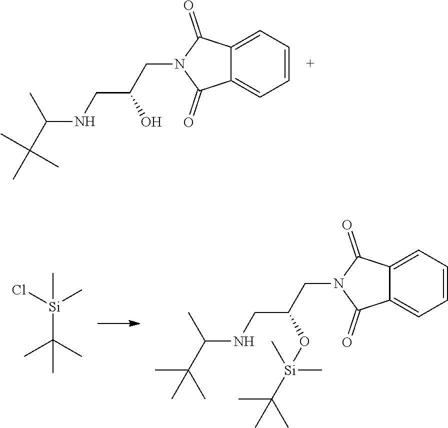

Into a solution of 2-[(2R)-2-hydroxy-3-(1,2,2-trimethylpropylamino)propyl]isoindoline-1,3-dione (15.588 g, 50.187 mmol) and imidazole (6.8329 g, 100.37 mmol) in DMF (155.88 mL) was added tert-butyl-chloro-dimethyl-silane (15.128 g, 100.37 mmol). The reaction mixture was stirred at room temperature for 4 days. The reaction mixture was quenched with brine (250 mL), and the water layer was extracted with ethyl acetate (3×250 mL). The combined organic layers were washed with brine (3×250 mL), dried over anhydrous sodium sulfate and concentrated under vacuum. The residue was purified by silica gel chromatography using 0 to 30% ethyl acetate in hexanes to furnish 2-[(2R)-2-[tert-butyl(dimethyl)silyl]oxy-3-(1,2,2-trimethylpropylamino)propyl]isoindoline-1,3-dione (20.693 g, 96%) as a light yellow liquid. The product is a mixture of diastereomers. ESI-MS m/z calc. 418.26517, found 419.2 (M+1)⁺; Retention time: 4.99 minutes; LC method S.

### Step 3: Ethyl 2-[[(2R)-2-[tert-butyl(dimethyl)silyl]oxy-3-(1,3-dioxoisoindolin-2-yl)propyl]-(1,2,2-trimethylpropyl)amino]acetate

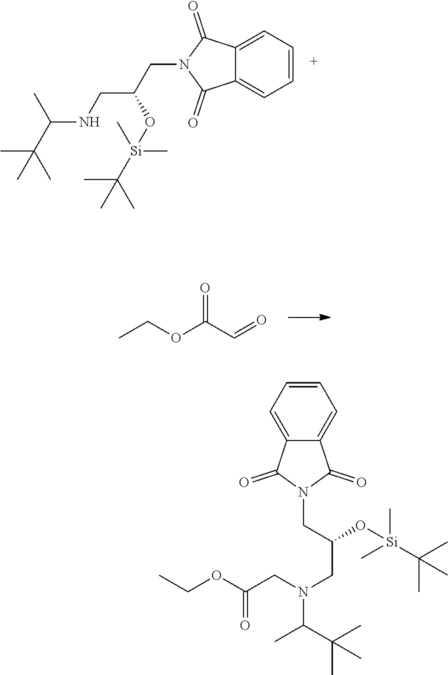

Into a solution of 2-[(2R)-2-[tert-butyl(dimethyl)silyl]oxy-3-(1,2,2-trimethylpropylamino)propyl]isoindoline-1,3-dione (10.83 g, 25.093 mmol) in dichloromethane (300 mL) was added acetic acid (7.5341 g, 7.1346 mL, 125.46 mmol). ethyl 2-oxoacetate (30.741 g, 50% w/w, 150.56 mmol) and sodium triacetoxyborohydride (53.182 g, 250.93 mmol) were added to the reaction mixture alternatively in several batches (within 2 hours), and the reaction mixture was left stirring at room temperature overnight. The reaction mixture was quenched with saturated sodium bicarbonate (300 mL), and stirred for 1 hour. Two layers were separated, and the aqueous layer was extracted with dichloromethane (2×300 mL). The combined dichloromethane layers were washed with brine (200 mL), dried over anhydrous sodium sulfate and concentrated under vacuum. The residue was purified by silica gel chromatography using 0 to 20% ethyl acetate in hexanes to furnish ethyl 2-[[(2R)-2-[tert-butyl(dimethyl)silyl]oxy-3-(1,3-dioxoisoindolin-2-yl)propyl]-(1,2,2-trimethylpropyl)amino]acetate (8.431 g, 65%) as a clear oil (mixture of diastereomers). ESI-MS m/z calc. 504.3019, found 505.3 (M+1)⁺; Retention time: 5.87 minutes (isomer A), Retention time: 6.44 (isomer B), LC method S

### Step 4: (6S)-6-[tert-Butyl(dimethyl)silyl]oxy-4-(1,2,2-trimethylpropyl)-1,4-diazepan-2-one

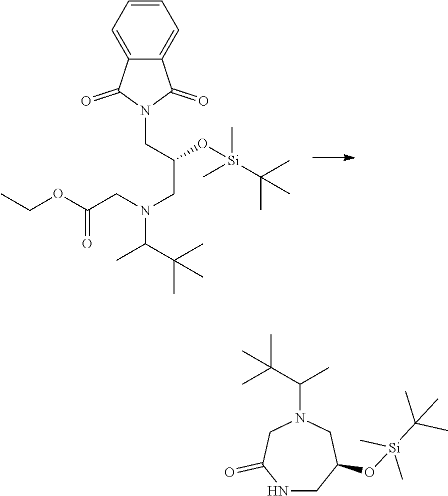 [SMILES: CCOC(=O)CN(C[C@H](CN1C(=O)c2ccccc2C1=O)O[Si](C)(C)C(C)(C)C)C(C)C(C)(C)C ; CC(N1CC(=O)NC[C@H](O[Si](C)(C)C(C)(C)C)C1)C(C)(C)C]

To a solution of ethyl 2-[[(2R)-2-[tert-butyl(dimethyl)silyl]oxy-3-(1,3-dioxoisoindolin-2-yl)propyl]-(1,2,2-trimethylpropyl)amino]acetate (8.431 g, 16.203 mmol) in ethanol (150 mL) was added hydrazine hydrate (5.5557 g, 81.015 mmol). The reaction was stirred at 80° C. for 4 days. After cooling down to room temperature, the solvent was removed under reduced pressure. The residue was diluted with 10% NaOH (aqueous) (100 mL), and extracted with ethyl acetate (3×100 mL). The combined organic layers were washed with brine (100 mL), dried over anhydrous sodium sulfate and concentrated under vacuum. The residue was purified by silica gel chromatography using 0 to 30% acetone in hexanes to furnish (6S)-6-[tert-butyl(dimethyl)silyl]oxy-4-(1,2,2-trimethylpropyl)-1,4-diazepan-2-one (4.4332 g, 79%) as a light yellow oil. The product is a mixture of diastereomers. ESI-MS m/z calc. 328.2546, found 329.1 (M+1)⁺; Retention time: 4.19 minutes; LC method S.

### Step 5: tert-Butyl (6R)-6-hydroxy-4-(1,2,2-trimethylpropyl)-1,4-diazepane-1-carboxylate

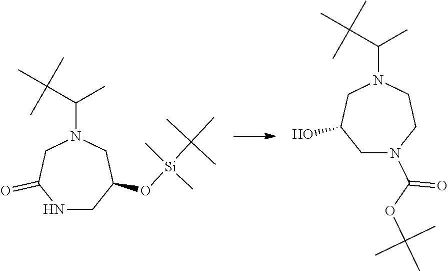 [SMILES: CC(N1CC(=O)NC[C@H](O[Si](C)(C)C(C)(C)C)C1)C(C)(C)C ; CC(N1CCN(C(=O)OC(C)(C)C)C[C@H](O)C1)C(C)(C)C]

Stage 1: Into a solution of (6S)-6-[tert-butyl(dimethyl)silyl]oxy-4-(1,2,2-trimethylpropyl)-1,4-diazepan-2-one (4.4332 g, 12.818 mmol) in anhydrous THE (50 mL) was added LAH (2.9190 g, 76.908 mmol) slowly at 0° C. The reaction mixture was stirred at 40° C. overnight. The reaction was cooled to 0° C. in an ice batch, and it was diluted with diethyl ether (50 mL). The reaction was quenched with water (3 mL), 15% NaOH (3 mL) and water (9 mL), and it was stirred at room temperature for 30 minutes. The white precipitate was removed by filtration through a pad of Celite and washed with THE (3×10 mL). The combined filtrate was concentrated under vacuum.

Stage 2: The residue was dissolved in THE (30 mL), and a aqueous solution of NaOH (999.93 g, 25 mL of 10% w/w, 2.5000 mol) was added, followed by Boc anhydride (4.1962 g, 4.4171 mL, 19.227 mmol). The reaction was stirred at room temperature for 1 hour. The volatile was removed under vacuum. The aqueous residue was extracted with ethyl acetate (3×50 mL). The combined organic phases were washed with brine (50 mL), dried over anhydrous sodium sulfate and concentrated under vacuum. The residue was purified by silica gel chromatography using 0 to 50% ethyl acetate in hexane to furnish tert-butyl (6R)-6-hydroxy-4-(1,2,2-trimethylpropyl)-1,4-diazepane-1-carboxylate (2.3853 g, 61%) as a clear oil. ¹H NMR (250 MHz, DMSO-d₆) δ 4.77-4.61 (m, 1H), 3.90-3.44 (m, 3H), 3.09-2.13 (m, 8H), 1.38 (s, 9H), 0.98-0.88 (m, 3H), 0.85 (s, 4H), 0.83 (s, 5H). The product is a mixture of diastereomers. ESI-MS m/z calc. 300.2413, found 301.6 (M+1)⁺; Retention time: 1.54 minutes; LC method W.

### Step 6: (6S)-1-(1,2,2-Trimethylpropyl)-1,4-diazepan-6-ol

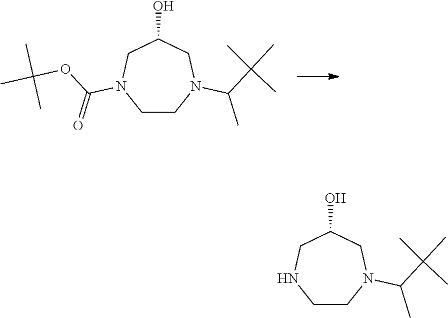 [SMILES: CC(N1CCN(C(=O)OC(C)(C)C)C[C@H](O)C1)C(C)(C)C ; CC(N1CCNC[C@H](O)C1)C(C)(C)C]

To a stirred solution of tert-butyl (6R)-6-hydroxy-4-(1,2,2-trimethylpropyl)-1,4-diazepane-1-carboxylate (1.10 g, 3.661 mmol) in anhydrous dioxane (12 mL) was added 4 M hydrogen chloride in dioxane (9.2 mL of 4 M, 36.80 mmol) at ambient temperature under nitrogen. The orange solution was stirred for 1 h, then the volatiles were removed under reduced pressure. To the residue, toluene (20 mL) was added, concentrated under reduced pressure. The process was repeated thrice with toluene and dried under vacuum to dryness for 4 h. The crude material was taken directly to the next step without any purification. (6S)-1-(1,2,2-Trimethylpropyl)-1,4-diazepan-6-ol (dihydrochloride salt) (1.00 g, 100%). ¹H NMR (499 MHz, DMSO-d₆) δ 4.65-4.36 (m, 1H), 4.14-4.00 (m, 1H), 3.75-3.62 (m, 3H), 3.53-3.35 (m, 3H), 3.31-3.15 (m, 2H), 1.36-1.23 (m, 3H), 1.07 (s, 9H).

### Step 7: N-[4-Chloro-6-(2,6-dimethylphenyl)pyrimidin-2-yl]-3-[(6R)-6-hydroxy-4-(1,2,2-trimethylpropyl)-1,4-diazepane-1-carbonyl]benzenesulfonamide

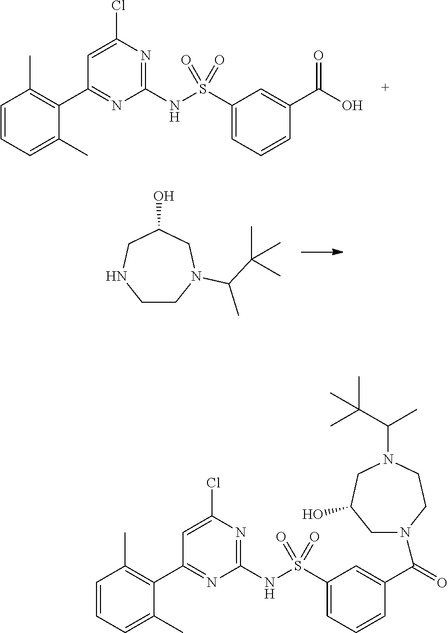 [SMILES: Cc1cccc(C)c1-c1cc(Cl)nc(NS(=O)(=O)c2cccc(C(=O)O)c2)n1 ; CC(N1CCNC[C@H](O)C1)C(C)(C)C ; Cc1cccc(C)c1-c1cc(Cl)nc(NS(=O)(=O)c2cccc(C(=O)N3CCN(C(C)C(C)(C)C)C[C@@H](O)C3)c2)n1]

A 100 mL flask was charged under nitrogen with (6S)-1-(1,2,2-trimethylpropyl)-1,4-diazepan-6-ol (Dihydrochloride salt) (500 mg, 1.830 mmol), anhydrous DMF (15 mL) and 3-[[4-chloro-6-(2,6-dimethylphenyl)pyrimidin-2-yl]sulfamoyl]benzoic acid (690 mg, 1.651 mmol). After dissolution of the reagents, the mixture was cooled down in an ice bath. N,N-diisopropyl ethyl amine (1.8 mL, 10.33 mmol) and HATU (765 mg, 2.012 mmol) were added to the reaction and the mixture was stirred at 0° C. for 10 min. The reaction was quenched by being poured into citric acid (50 mL of 10% w/v, 26.02 mmol)(10% aqueous) under vigorous stirring while cooled in an ice bath. The resulting white solid was not filtered due to poor formation of solid. The product was extracted with ethyl acetate (3×30 mL). The combined organics were washed with brine (20 mL), dried over anhydrous sodium sulfate, filtered and concentrated to a yellowish solid. The crude solid was dissolved in DCM (2 mL) and purified by flash chromatography (120 g silica gel column) using 0-5% methanol in dichloromethane over 30 min. The product eluted around 2-3% methanol. Evaporation of the volatiles and further drying in vacuo furnished N-[4-chloro-6-(2,6-dimethylphenyl)pyrimidin-2-yl]-3-[(6R)-6-hydroxy-4-(1,2,2-trimethylpropyl)-1,4-diazepane-1-carbonyl]benzenesulfonamide (hydrochloride salt) (721 mg, 69%) as an off-white foamy solid. ¹H NMR (499 MHz, DMSO-d₆) δ 12.36 (s, 1H), 7.98-7.84 (m, 2H), 7.64 (q, J=6.9 Hz, 1H), 7.58 (q, J=8.2 Hz, 1H), 7.30 (d, J=4.8 Hz, 1H), 7.24 (td, J=7.7, 2.3 Hz, 1H), 7.10 (dd, J=7.6, 5.1 Hz, 2H), 5.75 (s, 1H), 4.89 (s, 1H), 3.82-3.67 (m, 1H), 3.40 (dd, J=14.1, 5.1 Hz, 1H), 3.21-2.96 (m, 2H), 2.93-2.79 (m, 2H), 2.66-2.55 (m, 1H), 2.46-2.38 (m, 1H), 2.23-2.11 (m, 1H), 1.93-1.82 (m, 6H), 0.98-0.85 (m, 7H), 0.74 (s, 5H). ESI-MS m/z calc. 599.2333, found 600.4 (M+1)⁺; Retention time: 1.4 minutes; LC method A.

### Step 8: (16R)-18-(3,3-Dimethylbutan-2-yl)-12-(2,6-dimethylphenyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione, diastereomer 1 (Compound 72), and (16R)-18-(3,3-dimethylbutan-2-yl)-12-(2,6-dimethylphenyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione, diastereomer 2 (Compound 73)

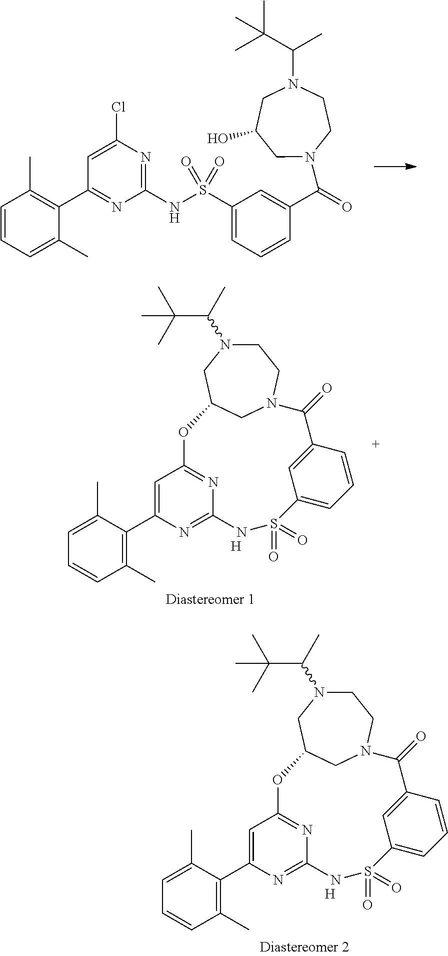

A 250 mL flask was charged under nitrogen with N-[4-chloro-6-(2,6-dimethylphenyl)pyrimidin-2-yl]-3-[(6R)-6-hydroxy-4-(1,2,2-trimethylpropyl)-1,4-diazepane-1-carbonyl]benzenesulfonamide (hydrochloride salt) (500 mg, 0.7854 mmol) and anhydrous DMF (25 mL). The mixture was cooled down in an ice-bath. Sodium hydride (300 mg of 60% w/w, 7.501 mmol) (60% mineral oil dispersion) was added in almost two equal portions. The mixture was stirred under nitrogen at 0° C. for 7 h. The ice-water bath was removed and allowed the reaction to warm to ambient temperature over 30 min and stirring continued for another 2.5 hours (total 7 h). The mixture was slowly poured into an ice-cold citric acid (100 mL of 10% w/v, 52.05 mmol) (aqueous 10% solution) under stirring. The resulting emulsion was extracted with EtOAc (4×50 mL). The combined organics were successively washed with water (50 mL) and brine (50 mL), then dried over anhydrous sodium sulfate and filtered. Evaporation of the volatiles under reduced pressure provided a residue (450 mg) that was purified by silica gel chromatography (40 g column) using 0 to 5% methanol in dichloromethane over 25 min, then a second time over 35 min gradient to give 198 mg of solid. Purification by preparative reverse-phase HPLC (C₁₈, 1-99% acetonitrile in water (containing 5 mM HCl) over 15 min provided two diastereomers: First to elute, diastereomer 1, (16R)-18-(3,3-dimethylbutan-2-yl)-12-(2,6-dimethylphenyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione (hydrochloride salt) (84 mg, 36%). ¹H NMR (500 MHz, DMSO-d₆) δ 8.86-8.69 (m, 1H), 7.99-7.86 (m, 1H), 7.78-7.58 (m, 2H), 7.26 (d, J=8.1 Hz, 1H), 7.14 (d, J=7.6 Hz, 2H), 6.35 (s, 1H), 6.08-5.92 (m, 1H), 4.45-4.30 (m, 1H), 4.23-4.07 (m, 1H), 3.91-3.74 (m, 2H), 3.73-3.51 (m, 5H), 2.06 (s, 6H), 1.40 (d, J=6.7 Hz, 2H), 1.11 (s, 7H), 0.92 (s, 3H). (one of the Me protons embedded in one of the tBu peaks). ESI-MS m/z calc. 563.25665, found 564.3 (M+1)⁺; Retention time: 1.39 minutes, (LC method A); and a second to elute, diastereomer 2, (16R)-18-(3,3-dimethylbutan-2-yl)-12-(2,6-dimethylphenyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione (hydrochloride salt) (77 mg, 33%). ESI-MS m/z calc. 563.25665, found 564.4 (M+1)⁺; Retention time: 1.52 minutes (LC method A).

### Example 26: Preparation of Compound 74

### Step 1: Benzyl (6R)-6-hydroxy-4-(2-nitrophenyl)sulfonyl-1,4-diazepane-1-carboxylate

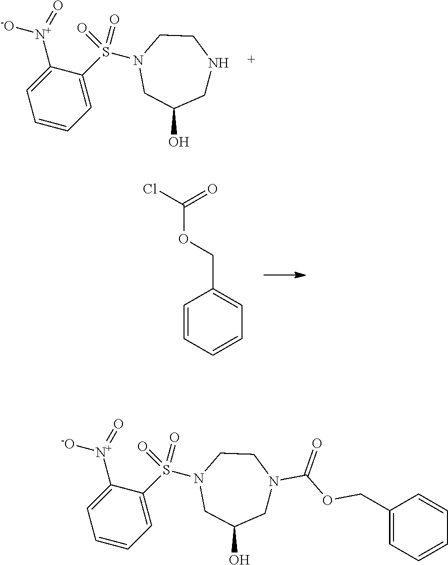

In a 100 mL vial, to a solution of (6R)-1-(2-nitrophenyl)sulfonyl-1,4-diazepan-6-ol (1.92 g, 6.372 mmol) in anhydrous dichloromethane (30 mL), was added triethylamine (1.4 mL, 10.04 mmol), followed by dropwise addition of benzyl chloroformate (1.1 mL, 7.705 mmol) under nitrogen at 0-5° C. (ice-water bath). The reaction was allowed to warm up to room temperature gradually and stirring was continued overnight (12 h). The volatiles were removed under reduced pressure and the residue was treated with dichloromethane (30 mL) and water (20 mL). The layers were separated, and the aqueous layer was extracted once more with dichloromethane (20 mL). The combined organic layers were dried (over sodium sulfate), filtered, and concentrated under reduced pressure. The crude material was purified by silica gel (40 g) column chromatography eluting with 0-5% methanol in dichloromethane over 20 min to furnish the desired benzyl (6R)-6-hydroxy-4-(2-nitrophenyl)sulfonyl-1,4-diazepane-1-carboxylate (2.43 g, 88%) as a light orange gum. ¹H NMR (400 MHz, Methanol-d₄) δ 8.02 (ddd, J=9.2, 7.6, 1.7 Hz, 1H), 7.84-7.75 (m, 3H), 7.45-7.22 (m, 5H), 5.14 (two d, J=12.2 Hz, 2×1H), 4.00-3.91 (m, 2H), 3.86 (dd, J=13.5, 7.1 Hz, 1H), 3.71 (td, J=12.8, 12.3, 5.4 Hz, 2H), 3.50-3.38 (m, 1H), 3.38-3.33 (m, 0.5H), 3.29-3.25 (m, 0.5H), 3.24-3.09 (m, 2H). ESI-MS m/z calc. 435.11002, found 436.3 (M+1)⁺; Retention time: 1.36 minutes; LC method A.

### Step 2: Benzyl (6S)-6-hydroxy-1,4-diazepane-1-carboxylate

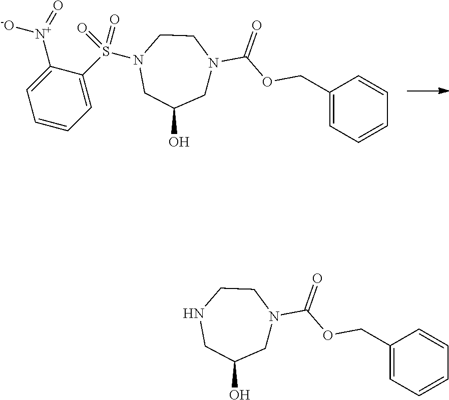

To a stirred solution of benzyl (6R)-6-hydroxy-4-(2-nitrophenyl)sulfonyl-1,4-diazepane-1-carboxylate (2.420 g, 5.557 mmol) in anhydrous DMF (20 mL) were added potassium carbonate (5.0 g, 36.18 mmol) and thiophenol (2.0 mL, 19.48 mmol), in that order, at ambient temperature under nitrogen. After stirring at that temperature for 1 h, the solid was removed by filtration and the filtrate was concentrated under reduced pressure. The crude was dry-loaded onto Celite and purified from silica gel chromatography [40 g silica gel, gradient elution with 0 to 15% methanol in methylene chloride (monitored by ELSD). The desired compound benzyl (6S)-6-hydroxy-1,4-diazepane-1-carboxylate (1.0 g, 72%) was obtained as glassy material. ¹H NMR (400 MHz, Methanol-d₄) δ 7.40-7.28 (m, 5H), 5.16 (d, J=12.5 Hz, 1H), 5.13 (d, J=12.5 Hz, 1H), 3.94 (dtd, J=10.2, 5.9, 5.2, 2.4 Hz, 1H), 3.78 (ddd, J=14.3, 4.8, 2.6 Hz, 1H), 3.68 (dtd, J=14.0, 6.8, 4.5 Hz, 1H), 3.51-3.42 (m, 1H), 3.42-3.35 (m, 1H), 3.04-2.96 (m, 1H), 2.94 (d, J=3.3 Hz, 1H), 2.88 (dddd, J=12.6, 7.0, 5.4, 2.8 Hz, 2H). ESI-MS m/z calc. 250.13174, found 251.2 (M+1)⁺; Retention time: 0.63 minutes (LC method A).

### Step 3: Benzyl (6S)-4-[3-[[4-chloro-6-(2,6-dimethylphenyl)pyrimidin-2-yl]sulfamoyl]benzoyl]-6-hydroxy-1,4-diazepane-1-carboxylate

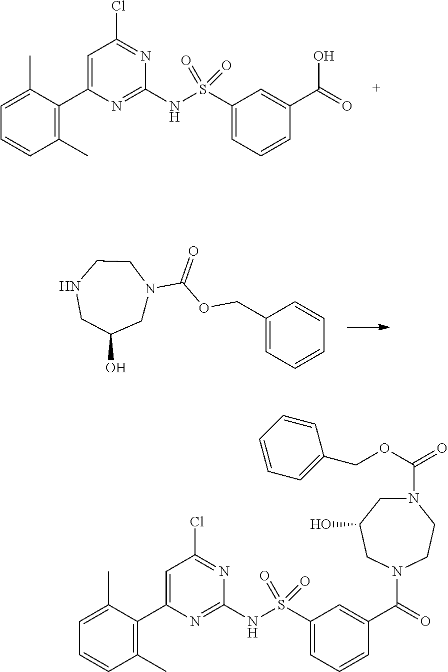 [SMILES: Cc1cccc(C)c1-c1cc(Cl)nc(NS(=O)(=O)c2cccc(C(=O)O)c2)n1 ; O=C(OCc1ccccc1)N1CCNC[C@H](O)C1 ; Cc1cccc(C)c1-c1cc(Cl)nc(NS(=O)(=O)c2cccc(C(=O)N3CCN(C(=O)OCc4ccccc4)C[C@@H](O)C3)c2)n1]

To a stirred solution of 3-[[4-chloro-6-(2,6-dimethylphenyl)pyrimidin-2-yl]sulfamoyl]benzoic acid (1.01 g, 2.366 mmol) in anhydrous DMF (15 mL) was added diisopropylethylamine (2.1 mL, 12.06 mmol) at 0-5° C. under nitrogen, followed by the addition of HATU (945 mg, 2.485 mmol). After stirring for 2 min, a solution of benzyl (6S)-6-hydroxy-1,4-diazepane-1-carboxylate (592 mg, 2.365 mmol) in anhydrous DMF (2 mL) was added. The reaction mixture was stirred for 10 min then poured in 10% aqueous citric acid solution (75 mL) and extracted with ethyl acetate (30 mL). The aqueous layer was re-extracted with ethyl acetate (2×25 mL) and the combined organic layers were washed with brine (40 mL), dried over anhydrous sodium sulfate, filtered and concentrated under reduced pressure. The residue was purified by silica gel column chromatography (40 g column) using 0-10% methanol (desired peak came around 5% methanol) to furnish benzyl (6S)-4-[3-[[4-chloro-6-(2,6-dimethylphenyl)pyrimidin-2-yl]sulfamoyl]benzoyl]-6-hydroxy-1,4-diazepane-1-carboxylate (1.025 g, 67%) as a white solid. ESI-MS m/z calc. 649.1762, found 650.4 (M+1)⁺; Retention time: 1.74 minutes; LC method A.

### Step 4: Benzyl (16S)-12-(2,6-dimethylphenyl)-2,8,8-trioxo-15-oxa-8λ⁶-thia-1,9,11,18,22-pentaazatetracyclo[14.4.1.13,7.110,14]tricosa-3,5,7(23),10,12,14(22)-hexaene-18-carboxylate

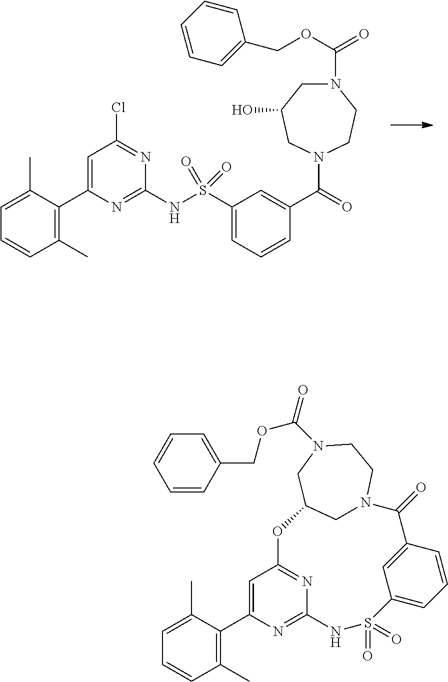 [SMILES: Cc1cccc(C)c1-c1cc(Cl)nc(NS(=O)(=O)c2cccc(C(=O)N3CCN(C(=O)OCc4ccccc4)C[C@@H](O)C3)c2)n1 ; Cc1cccc(C)c1-c1cc2nc(n1)NS(=O)(=O)c1cccc(c1)C(=O)N1CCN(C(=O)OCc3ccccc3)C[C@H](C1)O2]

To a stirred solution of benzyl (6S)-4-[3-[[4-chloro-6-(2,6-dimethylphenyl)pyrimidin-2-yl]sulfamoyl]benzoyl]-6-hydroxy-1,4-diazepane-1-carboxylate (989 mg, 1.521 mmol) in anhydrous DMF (50 mL) was added sodium hydride (730 mg, 18.25 mmol) in one portion at 5° C. (ice-water bath) under nitrogen. The reaction mixture was stirred at that temperature for 2 h, then poured over 10% aqueous citric acid solution (60 mL). The product was extracted with ethyl acetate (3×40 mL) and the combined organic layers were washed with brine (3×40 mL), dried over anhydrous sodium sulfate, filtered, and concentrated under reduced pressure. The residue was purified by silica gel column chromatography (80 g column) using 0-5% methanol in dichloromethane over 35 min to furnish benzyl (16S)-12-(2,6-dimethylphenyl)-2,8,8-trioxo-15-oxa-8λ⁶-thia-1,9,11,18,22-pentaazatetracyclo[14.4.1.13,7.110,14]tricosa-3,5,7(23),10,12,14(22)-hexaene-18-carboxylate (411 mg, 44%) as a white solid. ¹H NMR (400 MHz, Methanol-d₄) δ 8.46 (two broad s, 1H), 8.05-7.98 (m, 1H), 7.72-7.64 (m, 2H), 7.45 (d, J=7.6 Hz, 1H), 7.39 (d, J=7.1 Hz, 1H), 7.31 (td, J=8.3, 7.8, 2.7 Hz, 2H), 7.27-7.18 (m, 2H), 7.13 (d, J=7.6 Hz, 2H), 6.23 (two s, 1H), 5.70-5.51 (m, 1H), 5.27 (dd, J=17.7, 12.3 Hz, 1H), 5.17 (dd, J=15.9, 11.6 Hz, 1H), 4.69-4.54 (m, 1H), 4.34 (dd, J=38.7, 14.4, 4.4 Hz, 1H), 4.18-4.05 (m, 1H), 3.76 (t, J=13.8 Hz, 1H), 3.69-3.55 (m, 1H), 3.54-3.46 (m, 1H), 3.28-3.15 (m, 2H), 2.10 (s, 6H). ESI-MS m/z calc. 613.1995, found 614.4 (M+1)⁺; Retention time: 1.56 minutes; LC method A.

### Step 5: (16R)-12-(2,6-Dimethylphenyl)-18-ethyl-15-oxa-8λ⁶-thia-1,9,11,18,22-pentaazatetracyclo[14.4.1.13,7.110,14]tricosa-3(23),4,6,10,12,14(22)-hexaene-2,8,8-trione (Compound 74)

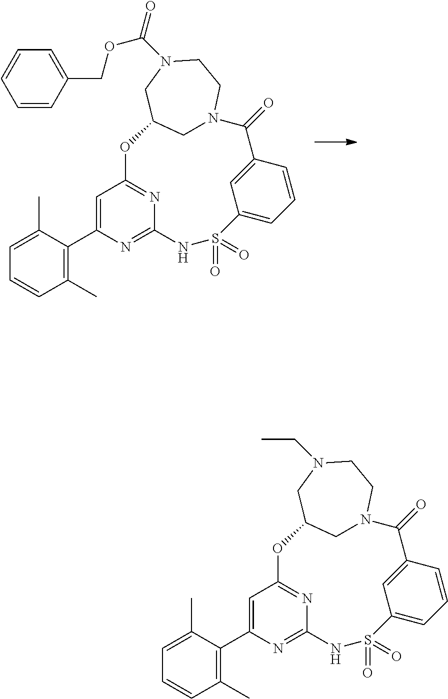 [SMILES: Cc1cccc(C)c1-c1cc2nc(n1)NS(=O)(=O)c1cccc(c1)C(=O)N1CCN(C(=O)OCc3ccccc3)C[C@H](C1)O2 ; CCN1CCN2C[C@@H](C1)Oc1cc(-c3c(C)cccc3C)nc(n1)NS(=O)(=O)c1cccc(c1)C2=O]

A heterogeneous mixture of benzyl (16S)-12-(2,6-dimethylphenyl)-2,8,8-trioxo-15-oxa-8λ⁶-thia-1,9,11,18,22-pentaazatetracyclo[14.4.1.13,7.110,14]tricosa-3,5,7(23),10,12,14(22)-hexaene-18-carboxylate (389 mg, 0.6339 mmol) in ethanol (20 mL) was sonicated for 10 min to get an emulsion (milky). It was degassed and palladium (68 mg, 0.06390 mmol) was added under nitrogen. The mixture was stirred under a hydrogen balloon at ambient temperature for 2 h. The flask was purged with nitrogen and more palladium (68 mg, 0.06390 mmol) was added and the reaction was continued under hydrogen for 36 h. The flask was purged with nitrogen and the solid catalyst was filtered off over a pad of Celite. The filtrate was concentrated and the residue was purified from silica gel chromatography [80 g silica gel column, 0-5% methanol in dichloromethane over 40 min (very steep gradient) and the product came around 4.2% methanol] to give (16R)-12-(2,6-dimethylphenyl)-15-oxa-8λ⁶-thia-1,9,11,18,22-pentaazatetracyclo[14.4.1.13,7.110,14]tricosa-3,5,7(23),10,12,14(22)-hexaene-2,8,8-trione (189 mg, 62%) as a white solid. ¹H NMR (400 MHz, Methanol-d₄) δ 8.71 (t, J=1.7 Hz, 1H), 8.00 (dt, J=7.0, 1.8 Hz, 1H), 7.73-7.61 (m, 2H), 7.26 (t, J=7.6 Hz, 1H), 7.13 (d, J=7.6 Hz, 2H), 5.61 (tt, J=9.6, 4.5 Hz, 1H), 4.37 (dt, J=13.8, 6.6 Hz, 1H), 3.72 (dd, J=14.5, 4.0 Hz, 1H), 3.51-3.41 (m, 1H), 3.37-3.32 (m, 1H), 3.29-3.21 (m, 2H), 3.10-2.99 (m, 2H), 2.10 (s, 6H). ESI-MS m/z calc. 479.16272, found 480.4 (M+1)⁺; Retention time: 0.76 minutes (LC method A).

The N-ethyl side product eluted first, at around 4% methanol in dichloromethane. The fractions were concentrated to obtain about 85% pure material. It was further purified by preparative reverse-phase HPLC (1-70% acetonitrile in water over 30 min, 5% HCl as modifier) to furnish (16R)-12-(2,6-dimethylphenyl)-18-ethyl-15-oxa-8λ⁶-thia-1,9,11,18,22-pentaazatetracyclo[14.4.1.13,7.110,14]tricosa-3(23),4,6,10,12,14(22)-hexaene-2,8,8-trione (hydrochloride salt) (16 mg, 5%) as a white solid. ¹H NMR (400 MHz, DMSO-d₆) δ 10.62 (s, 1H), 8.81 (s, 1H), 8.03-7.87 (m, 1H), 7.69 (d, J=4.8 Hz, 2H), 7.27 (t, J=7.6 Hz, 1H), 7.13 (d, J=7.6 Hz, 2H), 6.40 (s, 1H), 5.83 (tt, J=10.2, 4.2 Hz, 1H), 4.61-4.39 (m, 1H), 3.89 (q, J=7.2 Hz, 2H), 3.84 (d, J=4.1 Hz, 1H), 3.80 (d, J=4.1 Hz, 1H), 3.66-3.61 (m, 2H), 3.50 (dd, J=11.4, 5.8 Hz, 2H), 3.43-3.38 (m, 1H), 3.27 (dd, J=14.4, 10.9 Hz, 1H), 2.05 (s, 6H), 1.34 (t, J=7.1 Hz, 3H). ESI-MS m/z calc. 507.19403, found 508.3 (M+1)⁺; Retention time: 0.81 minutes (LC method A).

### Example 27: Preparation of Compound 75

### Step 1: (16R)-12-(2,6-Dimethylphenyl)-18-(propan-2-yl)-15-oxa-8λ⁶-thia-1,9,11,18,22-pentaazatetracyclo[14.4.1.13,7.110,14]tricosa-3,5,7(23),10,12,14(22)-hexaene-2,8,8-trione (Compound 75)

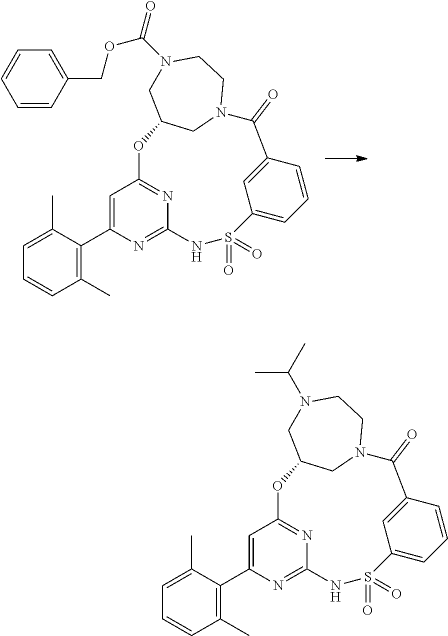 [SMILES: Cc1cccc(C)c1-c1cc2nc(n1)NS(=O)(=O)c1cccc(c1)C(=O)N1CCN(C(=O)OCc3ccccc3)C[C@H](C1)O2 ; Cc1cccc(C)c1-c1cc2nc(n1)NS(=O)(=O)c1cccc(c1)C(=O)N1CCN(C(C)C)C[C@H](C1)O2]

To a flask containing benzyl (16S)-12-(2,6-dimethylphenyl)-2,8,8-trioxo-15-oxa-8λ⁶-thia-1,9,11,18,22-pentaazatetracyclo[14.4.1.13,7.110,14]tricosa-3,5,7(23),10,12,14(22)-hexaene-18-carboxylate (1.03 g, 1.678 mmol) was added THE (50 mL), propan-2-ol (12.5 mL), Pd(OH)₂ (118 mg of 20% w/w, 0.1681 mmol), and acetic acid (5.0 mL, 87.92 mmol). The reaction was purged with nitrogen and the reaction was sparged with a balloon on hydrogen gas and allowed to stir overnight. More Pd(OH)₂ (118 mg of 20% w/w, 0.1681 mmol) was added and a balloon of hydrogen was used to sparge the reaction. More catalyst (118 mg of 20% w/w, 0.1681 mmol) was added and the reaction was complete in 2 more hours. The reaction was filtered over Celite and the filter cake was washed with isopropanol. The filtrate was evaporated to dryness. The residue was dissolved in DMSO and purified by reverse phase HPLC 1%-99% ACN:water with an HCl modifier to give (16R)-12-(2,6-dimethylphenyl)-15-oxa-8λ⁶-thia-1,9,11,18,22-pentaazatetracyclo[14.4.1.13,7.110,14]tricosa-3,5,7(23),10,12,14(22)-hexaene-2,8,8-trione as a white solid. A side product was isolated: (16R)-12-(2,6-dimethylphenyl)-18-(propan-2-yl)-15-oxa-8λ⁶-thia-1,9,11,18,22-pentaazatetracyclo[14.4.1.13,7.110,14]tricosa-3,5,7(23),10,12,14(22)-hexaene-2,8,8-trione (hydrochloride salt) (6.6 mg). ESI-MS m/z calc. 521.20966, found 522.2 (M+1)⁺; Retention time: 0.88 minutes; LC method A.

### Example 28: Preparation of Compound 76 and Compound 77

### Step 1: tert-Butyl N-(oxiran-2-ylmethyl)carbamate

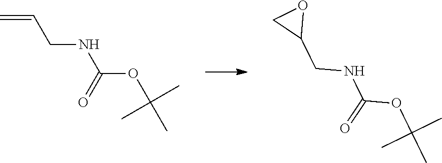

To a stirred solution of tert-butyl N-allylcarbamate (10.41 g, 66.217 mmol) in DCM (500 mL) at 0° C. was added portionwise m-CPBA (30.9 g, 134.30 mmol). The reaction mixture was allowed to warm up to room temperature overnight. The reaction was quenched with 10% aqueous sodium bisulfite (250 mL). Two layers were separated, and organic layer was washed with saturated aqueous sodium bicarbonate (150 mL) and brine (100 mL). The organic layer was dried over anhydrous sodium sulfate and concentrated to afford crude colorless oil of tert-butyl N-(oxiran-2-ylmethyl)carbamate (12.61 g, 93%). ¹H NMR (250 MHz, CDCl₃) δ 4.73 (s, 1H), 3.64-3.40 (m, 1H), 3.30-3.13 (m, 1H), 3.13-3.01 (m, 1H), 2.78 (dd, J=4.7, 4.0 Hz, 1H), 2.59 (dd, J=4.7, 2.6 Hz, 1H), 1.44 (s, 9H).

### Step 2: tert-Butyl N-[2-hydroxy-3-[(4-methoxyphenyl)methylamino]propyl]carbamate

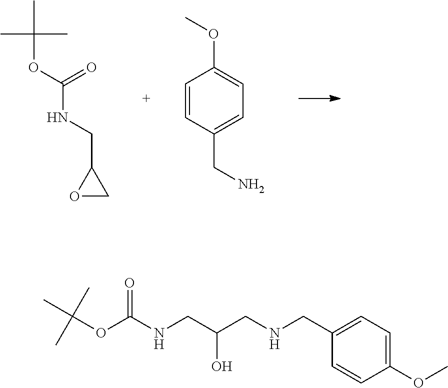

(4-Methoxyphenyl)methanamine (14.164 g, 103.25 mmol) was added to a solution of tert-butyl N-(oxiran-2-ylmethyl)carbamate (10.52 g, 51.625 mmol) in isopropanol (60 mL) and the mixture was stirred at 50° C. for 16 hours. Isopropanol was evaporated, toluene (100 mL) was added and the solution was evaporated to give crude tert-butyl N-[2-hydroxy-3-[(4-methoxyphenyl)methylamino]propyl]carbamate (25 g, 92%) as a yellow oil that was used in the next step without purification. ESI-MS m/z calc. 310.18927, found 311.4 (M+1)⁺; Retention time: 3.02 minutes; LC method S.

### Step 3: tert-Butyl N-[2-[tert-butyl(dimethyl)silyl]oxy-3-[(4-methoxyphenyl)methylamino]propyl]carbamate

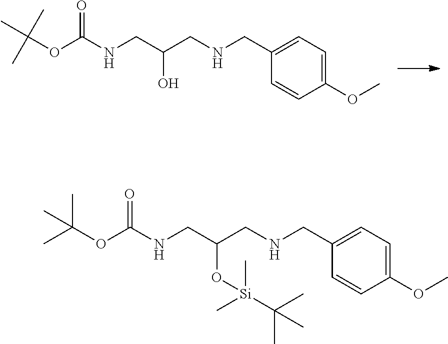

TBDMSCl (9.8150 g, 65.120 mmol) was added to a solution of tert-butyl N-[2-hydroxy-3-[(4-methoxyphenyl)methylamino]propyl]carbamate (24.47 g, 46.514 mmol) and TEA (12.143 g, 16.726 mL, 120.00 mmol) in 1,2-dichloroethane (120 mL) and the mixture was stirred at 60° C. for 24 hours. The mixture was diluted with chloroform (200 mL) and washed with saturated potassium carbonate (100 mL). The organic phase was separated, evaporated and the residue was purified by silica gel column chromatography using 0-30% hexanes-ethyl acetate to give tert-butyl N-[2-[tert-butyl(dimethyl)silyl]oxy-3-[(4-methoxyphenyl)methylamino]propyl]carbamate (16.43 g, 71%) as a colorless oil. ESI-MS m/z calc. 424.27573, found 425.4 (M+1)⁺; Retention time: 5.5 minutes; LC method S.

### Step 4: tert-Butyl N-[2-[tert-butyl(dimethyl)silyl]oxy-3-[(2-chloroacetyl)-[(4-methoxyphenyl)methyl]amino]propyl]carbamate

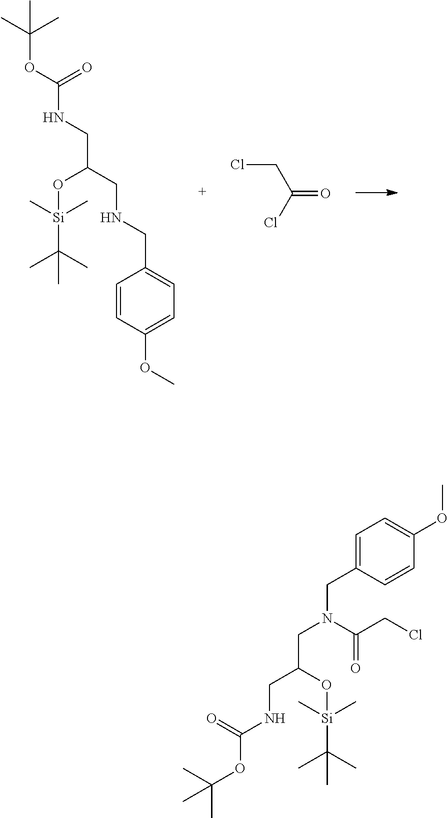 [SMILES: COc1ccc(CNCC(CNC(=O)OC(C)(C)C)O[Si](C)(C)C(C)(C)C)cc1 ; O=C(Cl)CCl ; COc1ccc(CN(CC(CNC(=O)OC(C)(C)C)O[Si](C)(C)C(C)(C)C)C(=O)CCl)cc1]

A solution of tert-butyl N-[2-[tert-butyl(dimethyl)silyl]oxy-3-[(4-methoxyphenyl)methylamino]propyl]carbamate (16.01 g, 37.702 mmol) and DIPEA (9.7454 g, 13.134 mL, 75.404 mmol) in DCM (370 mL) was cooled on ice-water bath and chloroacetyl chloride (5.5357 g, 3.8984 mL, 49.013 mmol) was added dropwise. The mixture was stirred for 1 hour, diluted with DCM (350 mL) and washed with 1M citric acid (300 mL). The organic phase was separated, dried over sodium sulfate and evaporated, the residue was purified by silica gel column chromatography using 0-20% hexane-ethyl acetate to give tert-butyl N-[2-[tert-butyl(dimethyl)silyl]oxy-3-[(2-chloroacetyl)-[(4-methoxyphenyl)methyl]amino]propyl]carbamate (17.208 g, 87%) as a colorless oil. ESI-MS m/z calc. 500.2473, found 501.6 (M+1)⁺; Retention time: 7.25 minutes; LC method S.

### Step 5: tert-Butyl 6-[tert-butyl(dimethyl)silyl]oxy-4-[(4-methoxyphenyl)methyl]-3-oxo-1,4-diazepane-1-carboxylate

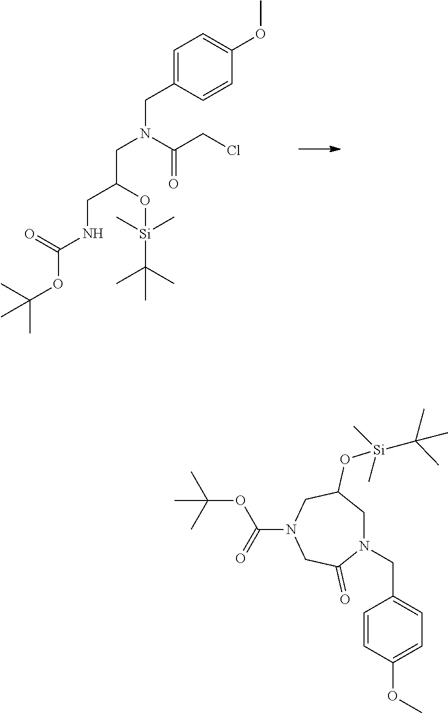 [SMILES: COc1ccc(CN(CC(CNC(=O)OC(C)(C)C)O[Si](C)(C)C(C)(C)C)C(=O)CCl)cc1 ; COc1ccc(CN2CC(O[Si](C)(C)C(C)(C)C)CN(C(=O)OC(C)(C)C)CC2=O)cc1]

tert-Butyl N-[2-[tert-butyl(dimethyl)silyl]oxy-3-[(2-chloroacetyl)-[(4-methoxyphenyl)methyl]amino]propyl]carbamate (6.208 g, 11.769 mmol) was dissolved in DMF (50 mL) and the solution was cooled on ice-water bath under argon. NaH (588.38 mg, 60% w/w, 14.711 mmol) was added portionwise, the mixture was allowed to warm up to room temperature and stirred overnight. The mixture was poured into a mixture of ethyl acetate (300 mL) and 1M citric acid (200 mL), the organic phase was separated, dried over sodium sulfate and evaporated, the residue was purified by silica gel column chromatography using 0-10% chloroform-methanol to give tert-butyl 6-[tert-butyl(dimethyl)silyl]oxy-4-[(4-methoxyphenyl)methyl]-3-oxo-1,4-diazepane-1-carboxylate (5.85 g, 86%) as a colorless oil. ESI-MS m/z calc. 464.27066, found 465.2 (M+1)⁺; Retention time: 3.86 minutes; LC method T.

### Step 6: tert-Butyl 6-hydroxy-4-[(4-methoxyphenyl)methyl]-3-oxo-1,4-diazepane-1-carboxylate

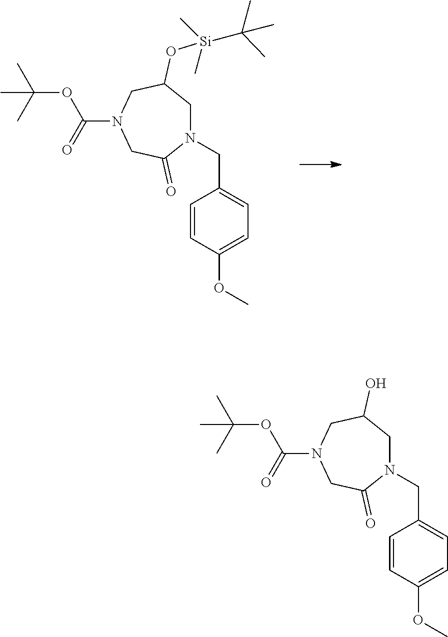

tert-Butyl 6-[tert-butyl(dimethyl)silyl]oxy-4-[(4-methoxyphenyl)methyl]-3-oxo-1,4-diazepane-1-carboxylate (5.85 g, 10.072 mmol) and acetic acid (703.09 mg, 0.6658 mL, 11.708 mmol) were dissolved in MeOH (100 mL) and KF (1.7554 g, 30.216 mmol) was added. The mixture was refluxed for 24 hours, evaporated and the residue was partitioned between dichloromethane (200 mL) and saturated potassium carbonate (30 mL). The organic phase was separated, dried over sodium sulfate and evaporated, the residue was purified by silica gel column chromatography using 0-3% chloroform-methanol to give tert-butyl 6-hydroxy-4-[(4-methoxyphenyl)methyl]-3-oxo-1,4-diazepane-1-carboxylate (3.421 g, 92%) as a colorless oil. ESI-MS m/z calc. 350.18417, found 351.1 (M+1)⁺; Retention time: 2.28 minutes; LC method T.

### Step 7: 3-[[4-[[4-tert-Butoxycarbonyl-1-[(4-methoxyphenyl)methyl]-2-oxo-1,4-diazepan-6-yl]oxy]-6-(2,6-dimethylphenyl)pyrimidin-2-yl]sulfamoyl]benzoic acid

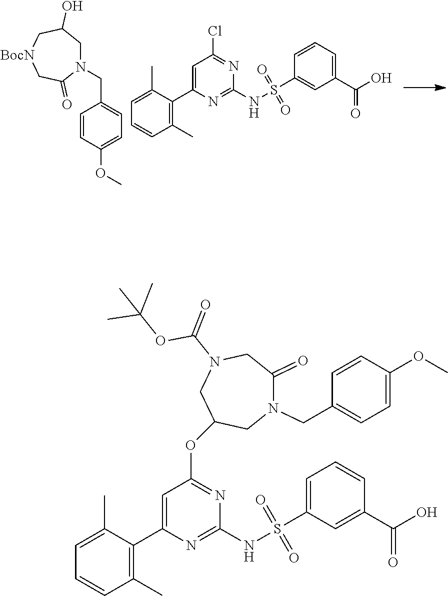

To a stirring solution of 3-[[4-chloro-6-(2,6-dimethylphenyl)pyrimidin-2-yl]sulfamoyl]benzoic acid (3.3747 g, 8.076 mmol) and tert-butyl 6-hydroxy-4-[(4-methoxyphenyl)methyl]-3-oxo-1,4-diazepane-1-carboxylate (2.97 g, 8.4758 mmol) in anhydrous DMF (40 mL) at 0° C. under nitrogen was added sodium hydride (3.2301 g, 60% w/w in mineral oil, 80.760 mmol). The reaction mixture was allowed to warm up to room temperature and stirred for 8 hours. The reaction was quenched with saturated aqueous ammonium chloride (80 mL). Brine was added (200 mL) and the aqueous layer was acidified to pH ˜3 with 10% aqueous citric acid. The product was extracted with ethyl acetate (3×120 mL). The combined organic layers were washed with brine (70 mL), dried over anhydrous sodium sulfate and concentrated. The product was purified by silica gel chromatography using 0-10% DCM-methanol to afford two fractions: 1) 3.08 g of reduced purity 3-[[4-[[4-tert-butoxycarbonyl-1-[(4-methoxyphenyl)methyl]-2-oxo-1,4-diazepan-6-yl]oxy]-6-(2,6-dimethylphenyl)pyrimidin-2-yl]sulfamoyl]benzoic acid (˜40% purity) and 2) pure 3-[[4-[[4-tert-butoxycarbonyl-1-[(4-methoxyphenyl)methyl]-2-oxo-1,4-diazepan-6-yl]oxy]-6-(2,6-dimethylphenyl)pyrimidin-2-yl]sulfamoyl]benzoic acid (2.04 g, 33%) as a white solid. Both isolated fractions were taken forward separately. ESI-MS m/z calc. 731.2625, found 732.8 (M+1)⁺; Retention time: 4.92 minutes; LC method S.

### Step 8: 3-[[4-[(1-tert-Butoxycarbonyl-3-oxo-1,4-diazepan-6-yl)oxy]-6-(2,6-dimethylphenyl)pyrimidin-2-yl]sulfamoyl]benzoic acid

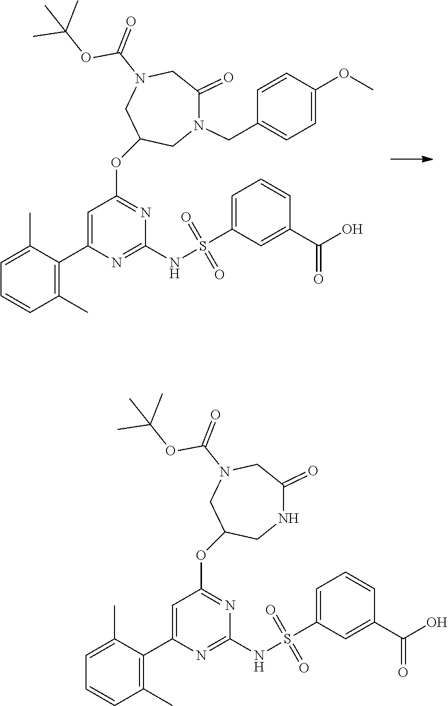 [SMILES: COc1ccc(CN2CC(Oc3cc(-c4c(C)cccc4C)nc(NS(=O)(=O)c4cccc(C(=O)O)c4)n3)CN(C(=O)OC(C)(C)C)CC2=O)cc1 ; Cc1cccc(C)c1-c1cc(OC2CNC(=O)CN(C(=O)OC(C)(C)C)C2)nc(NS(=O)(=O)c2cccc(C(=O)O)c2)n1]

To a stirring suspension of 3-[[4-[[4-tert-butoxycarbonyl-1-[(4-methoxyphenyl)methyl]-2-oxo-1,4-diazepan-6-yl]oxy]-6-(2,6-dimethylphenyl)pyrimidin-2-yl]sulfamoyl]benzoic acid (3.08 g, 1.6835 mmol) in Acetonitrile (40 mL) at 0° C. was added a solution of ceric ammonium nitrate (2.8044 g, 5.0505 mmol) in water (5 mL). The reaction mixture was allowed to warm up to room temperature and stirred for 4 hours. The reaction mixture was diluted with brine (100 mL) and acidified to pH ˜3 with 10% aqueous citric acid. The product was extracted with ethyl acetate (2×100 mL). The combined organic layers were dried over anhydrous sodium sulfate and concentrated. The crude was purified by silica gel chromatography using 0-10% chloroform-methanol to afford 3-[[4-[(1-tert-butoxycarbonyl-3-oxo-1,4-diazepan-6-yl)oxy]-6-(2,6-dimethylphenyl)pyrimidin-2-yl]sulfamoyl]benzoic acid (900 mg, 57%) ESI-MS m/z calc. 611.20496, found 612.6 (M+1)⁺; Retention time: 2.46 minutes; LC method T.

### Step 9: tert-Butyl 6-[6-(2,6-dimethylphenyl)-2-[[3-(hydroxymethyl)phenyl]sulfonylamino]pyrimidin-4-yl]oxy-3-oxo-1,4-diazepane-1-carboxylate

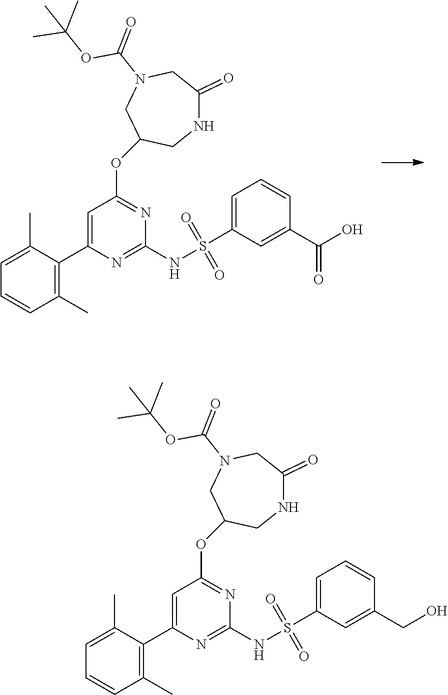 [SMILES: Cc1cccc(C)c1-c1cc(OC2CNC(=O)CN(C(=O)OC(C)(C)C)C2)nc(NS(=O)(=O)c2cccc(C(=O)O)c2)n1 ; Cc1cccc(C)c1-c1cc(OC2CNC(=O)CN(C(=O)OC(C)(C)C)C2)nc(NS(=O)(=O)c2cccc(CO)c2)n1]

To a stirring solution of 3-[[4-[(1-tert-butoxycarbonyl-3-oxo-1,4-diazepan-6-yl)oxy]-6-(2,6-dimethylphenyl)pyrimidin-2-yl]sulfamoyl]benzoic acid (1.52 g, 1.6153 mmol) and N-methylmorpholine (179.72 mg, 0.1953 mL, 1.7768 mmol) in anhydrous THF (30 mL) at −10° C. under nitrogen was dropwise added ethyl chloroformate (192.83 mg, 0.1699 mL, 1.7768 mmol). The reaction mixture was stirred for 1 hour, then warmed up to 0° C. and sodium borohydride (183.33 mg, 4.8459 mmol) was added. The reaction mixture was stirred at 0° C. for 4 hours. The reaction was quenched cold with water (20 mL), brine was added (50 mL) and volatiles were removed under vacuum. The aqueous layer was acidified to pH ˜3 with 10% aqueous citric acid and the product was extracted with ethyl acetate (2×75 mL). The combined organic layers were dried over anhydrous sodium sulfate and concentrated. The crude was purified by silica gel chromatography using 0-100% hexanes-ethyl acetate, followed by 0-10% chloroform-methanol to afford as white solid tert-butyl 6-[6-(2,6-dimethylphenyl)-2-[[3-(hydroxymethyl)phenyl]sulfonylamino]pyrimidin-4-yl]oxy-3-oxo-1,4-diazepane-1-carboxylate (635 mg, 62%). ESI-MS m/z calc. 597.2257, found 598.4 (M+1)⁺; Retention time: 3.31 minutes; LC method S.

### Step 10: tert-Butyl 6-[2-[[3-(bromomethyl)phenyl]sulfonylamino]-6-(2,6-dimethylphenyl)pyrimidin-4-yl]oxy-3-oxo-1,4-diazepane-1-carboxylate

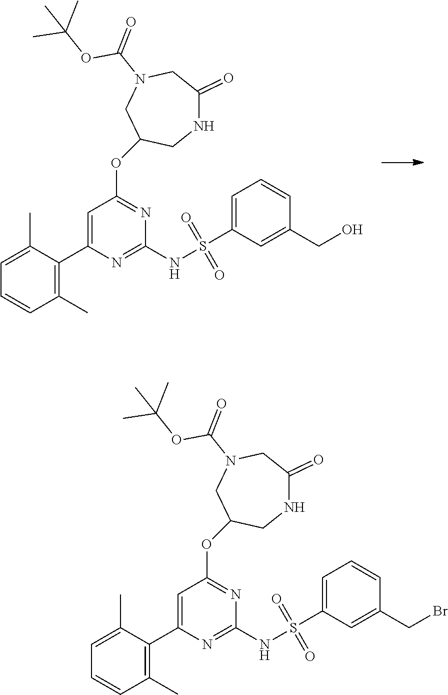 [SMILES: Cc1cccc(C)c1-c1cc(OC2CNC(=O)CN(C(=O)OC(C)(C)C)C2)nc(NS(=O)(=O)c2cccc(CO)c2)n1 ; Cc1cccc(C)c1-c1cc(OC2CNC(=O)CN(C(=O)OC(C)(C)C)C2)nc(NS(=O)(=O)c2cccc(CBr)c2)n1]

To a stirring solution of carbon tetrabromide (389.53 mg, 1.1746 mmol) and Triphenylphosphine (308.08 mg, 1.1746 mmol) in anhydrous DCM (25 mL) at 0° C. under nitrogen was added tert-butyl 6-[6-(2,6-dimethylphenyl)-2-[[3-(hydroxymethyl)phenyl]sulfonylamino]pyrimidin-4-yl]oxy-3-oxo-1,4-diazepane-1-carboxylate (585 mg, 0.9788 mmol). The reaction mixture was stirred at 0° C. for 2 hours and then quenched cold with saturated aqueous ammonium chloride (20 mL). After warming to room temperature, two layers were separated, and the organic layer was concentrated. The crude was purified by silica gel chromatography using 0-10% chloroform-methanol to afford as a white solid tert-butyl 6-[2-[[3-(bromomethyl)phenyl]sulfonylamino]-6-(2,6-dimethylphenyl)pyrimidin-4-yl]oxy-3-oxo-1,4-diazepane-1-carboxylate (311 mg, 41%). ESI-MS m/z calc. 659.1413, found 660.7 (M+1)⁺; Retention time: 5.7 minutes; LC method S.

### Step 11: tert-Butyl 12-(2,6-dimethylphenyl)-8,8,20-trioxo-15-oxa-8λ⁶-thia-1,9,11,18,22-pentaazatetracyclo[14.4.1.13,7.110,14]tricosa-3(23),4,6,10,12,14(22)-hexaene-18-carboxylate (Compound 77)

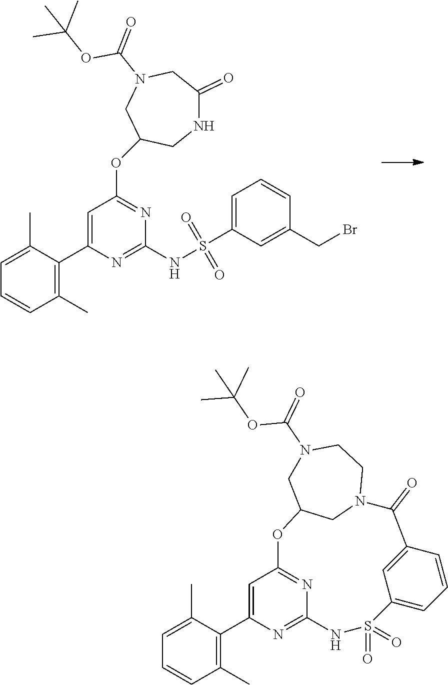 [SMILES: Cc1cccc(C)c1-c1cc(OC2CNC(=O)CN(C(=O)OC(C)(C)C)C2)nc(NS(=O)(=O)c2cccc(CBr)c2)n1 ; Cc1cccc(C)c1-c1cc2nc(n1)NS(=O)(=O)c1cccc(c1)C(=O)N1CCN(C(=O)OC(C)(C)C)CC(C1)O2]

To a stirring solution of tert-butyl 6-[2-[[3-(bromomethyl)phenyl]sulfonylamino]-6-(2,6-dimethylphenyl)pyrimidin-4-yl]oxy-3-oxo-1,4-diazepane-1-carboxylate (286 mg, 0.4330 mmol) in anhydrous DMF (22 mL) at 0° C. under nitrogen was added portionwise sodium hydride (173.18 mg, 60% w/w in mineral oil, 4.3300 mmol). The reaction mixture was stirred at 0° C. for 30 minutes and then slowly quenched cold by a dropwise addition of saturated aqueous ammonium chloride (30 mL). The product was extracted with ethyl acetate (2×75 mL). The combined organic layers were washed with brine (100 mL), dried over anhydrous sodium sulfate and concentrated. The crude was purified by silica gel chromatography using 0-65% hexanes-ethyl acetate to afford as white solid tert-butyl 12-(2,6-dimethylphenyl)-8,8,20-trioxo-15-oxa-8λ⁶-thia-1,9,11,18,22-pentaazatetracyclo[14.4.1.13,7.110,14]tricosa-3(23),4,6,10,12,14(22)-hexaene-18-carboxylate (177 mg, 68%). ¹H NMR (250 MHz, DMSO-d₆) δ 8.10 (s, 1H), 7.63 (d, J=40.0 Hz, 4H), 7.24 (t, J=7.6 Hz, 1H), 7.11 (d, J=7.5 Hz, 2H), 6.36 (s, 1H), 5.53 (d, J=18.5 Hz, 2H), 4.31-4.17 (m, 2H), 4.03 (t, J=15.0 Hz, 2H), 3.76 (t, J=12.4 Hz, 1H), 3.45 (s, 1H), 3.08-2.85 (m, 1H), 2.02 (s, 6H), 1.42 (s, 9H). ESI-MS m/z calc. 579.21515, found 580.3 (M+1)⁺; Retention time: 2.25 minutes; LC method T.

### Step 12: 12-(2,6-Dimethylphenyl)-15-oxa-8λ⁶-thia-1,9,11,18,22-pentaazatetracyclo[14.4.1.13,7.110,14]tricosa-3(23),4,6,10,12,14(22)-hexaene-8,8,20-trione (Compound 76)

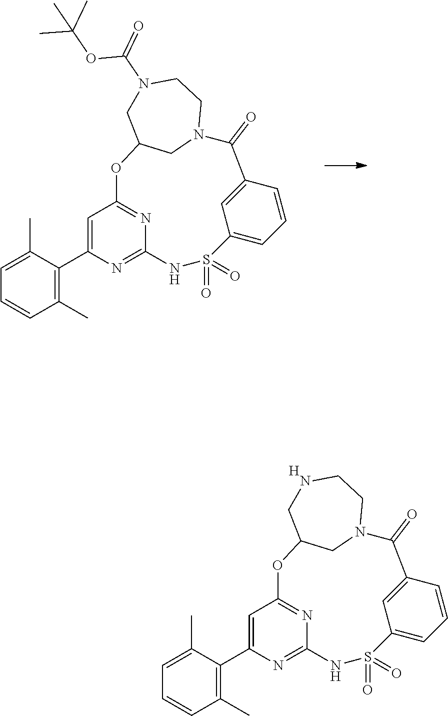

In a 20-mL vial, to a stirred solution of tert-butyl 12-(2,6-dimethylphenyl)-8,8,20-trioxo-15-oxa-8λ⁶-thia-1,9,11,18,22-pentaazatetracyclo[14.4.1.13,7.110,14]tricosa-3(23),4,6,10,12,14(22)-hexaene-18-carboxylate (100 mg, 0.1725 mmol) in anhydrous dioxane (2.0 mL) was added hydrogen chloride in dioxane (1.0 mL of 4.0 M, 4.000 mmol) at ambient temperature under nitrogen. After stirring for 1 h, the volatiles were removed under reduced pressure and the solid was dried under vacuum overnight. The desired 12-(2,6-dimethylphenyl)-15-oxa-8λ⁶-thia-1,9,11,18,22-pentaazatetracyclo[14.4.1.13,7.110,14]tricosa-3(23),4,6,10,12,14(22)-hexaene-8,8,20-trione (hydrochloride salt) (89 mg, 99%) was obtained as white solid. ¹H NMR (400 MHz, Methanol-d₄) δ 8.35 (d, J=1.9 Hz, 1H), 7.89-7.79 (m, 1H), 7.60-7.53 (m, 2H), 7.30-7.20 (m, 1H), 7.12 (d, J=7.4 Hz, 2H), 6.24 (s, 1H), 6.07 (tdd, J=11.0, 4.6, 2.4 Hz, 1H), 5.76 (d, J=16.2 Hz, 1H), 4.38 (d, J=15.1 Hz, 1H), 4.14-4.01 (m, 2H), 3.88 (td, J=10.2, 6.3 Hz, 2H), 3.76-3.66 (m, 1H), 3.65-3.55 (m, 1H), 3.51 (dd, J=12.5, 11.2 Hz, 1H), 2.07 (s, 6H). ESI-MS m/z calc. 479.16272, found 480.4 (M+1)⁺; Retention time: 0.8 minutes; LC method A.

### Example 29: Preparation of Compound 78

### Step 1: 18-(4,4-Difluorocyclohexyl)-12-(2,6-dimethylphenyl)-15-oxa-8λ⁶-thia-1,9,11,18,22-pentaazatetracyclo[14.4.1.13,7.110,14]tricosa-3(23),4,6,10,12,14(22)-hexaene-8,8,20-trione (Compound 78)

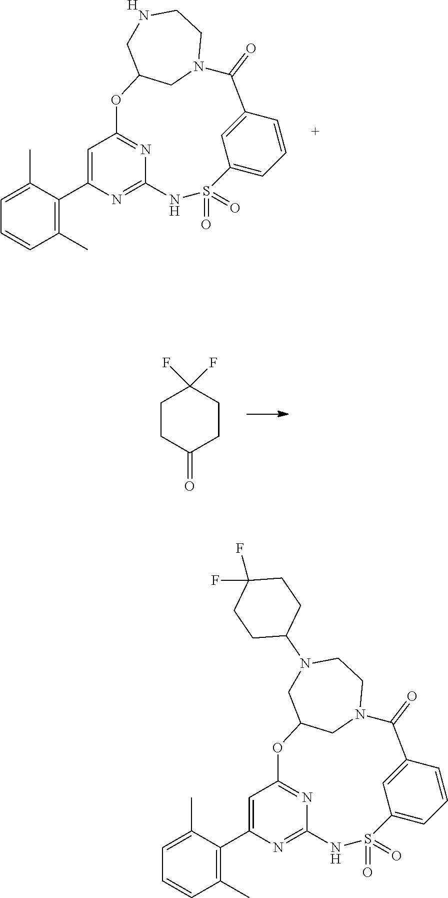

In a 4 mL vial, to a stirred solution of 4,4-difluorocyclohexanone (15 mg, 0.1118 mmol) in anhydrous 1,2-dichloroethane (1.0 mL) were added 12-(2,6-dimethylphenyl)-15-oxa-8λ⁶-thia-1,9,11,18,22-pentaazatetracyclo[14.4.1.13,7.110,14]tricosa-3(23),4,6,10,12,14(22)-hexaene-8,8,20-trione (hydrochloride salt) (12 mg, 0.02326 mmol), triethylamine (5 μL, 0.03587 mmol) and glacial acetic acid (5 μL, 0.08792 mmol), in that order. The resulting light-yellow solution was stirred at ambient temperature for 30 min, then sodium cyanoborohydride (14 mg, 0.2228 mmol) was added and stirring continued for 13 hours (overnight). The crude material was diluted with DMSO (0.8 mL), microfiltered, and purified by preparative reverse-phase HPLC eluting with 1-99% acetonitrile in water over 15 min (HCl as modifier). The desired product 18-(4,4-difluorocyclohexyl)-12-(2,6-dimethylphenyl)-15-oxa-8λ⁶-thia-1,9,11,18,22-pentaazatetracyclo[14.4.1.13,7.110,14]tricosa-3(23),4,6,10,12,14(22)-hexaene-8,8,20-trione (hydrochloride salt) (3.0 mg, 20%) was obtained as a white solid. ¹H NMR (400 MHz, Methanol-d₄) δ 8.31 (s, 1H), 7.83 (dd, J=6.6, 2.4 Hz, 1H), 7.60-7.51 (m, 2H), 7.29-7.22 (m, 1H), 7.12 (d, J=7.7 Hz, 2H), 6.22 (s, 1H), 6.11 (t, J=10.9 Hz, 1H), 5.79 (d, J=16.2 Hz, 1H), 4.45 (apparent q, J=14.5 Hz, 1H), 4.13 (d, J=8.3 Hz, 1H), 4.09 (d, J=9.6 Hz, 1H), 3.90 (dd, J=14.8, 10.4 Hz, 2H), 3.62 (t, J=11.7 Hz, 2H), 3.24 (d, J=14.7 Hz, 1H), 2.31-2.19 (m, 4H), 2.07 (s, 6H), 2.05-1.83 (m, 4H). ESI-MS m/z calc. 597.2221, found 598.5 (M+1)⁺; Retention time: 1.47 minutes; LC method A.

### Example 30: Preparation of Compound 79

### Step 1: (16R)-18-[2-(Adamantan-1-yl)acetyl]-12-(2,6-dimethylphenyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione (Compound 79)

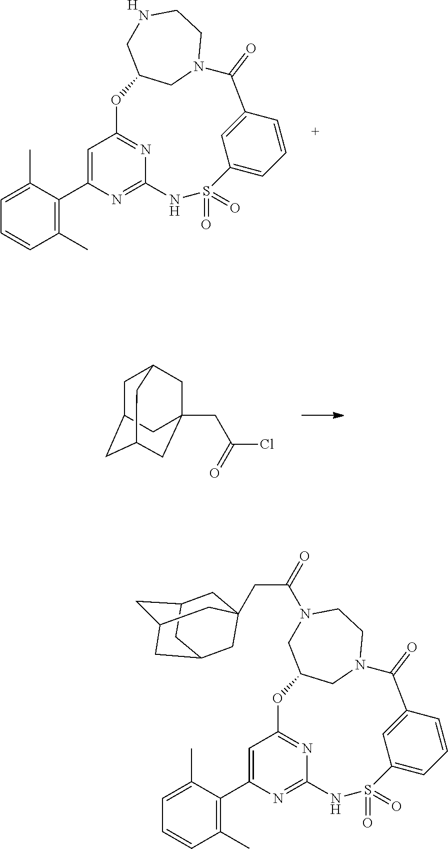

To a stirred solution of (16R)-12-(2,6-dimethylphenyl)-15-oxa-8λ⁶-thia-1,9,11,18,22-pentaazatetracyclo[14.4.1.13,7.110,14]tricosa-3,5,7(23),10,12,14(22)-hexaene-2,8,8-trione (hydrochloride salt) (12 mg, 0.02326 mmol) and N,N-diisopropyl ethyl amine (15 μL, 0.08612 mmol) in anhydrous dichloromethane (0.6 mL) was added 2-(1-adamantyl)acetyl chloride (6 mg, 0.02821 mmol) at 0-5° C. (ice-water bath) under nitrogen. The reaction was stirred at ambient temperature for 3 hours then concentrated under reduced pressure and the crude material was taken up in DMSO (1.0 mL), microfiltered, and purified by preparative reverse-phase HPLC eluting with 1-99% acetonitrile in water over 15 min (HCl as modifier). The desired product (16R)-18-[2-(adamantan-1-yl)acetyl]-12-(2,6-dimethylphenyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione (12.1 mg, 79%) was obtained as white solid. ¹H NMR (400 MHz, Methanol-d₄) δ 8.51 (t, J=1.7 Hz, 1H), 8.01 (ddt, J=6.9, 4.9, 1.8 Hz, 1H), 7.69 (tq, J=7.5, 4.0 Hz, 2H), 7.27 (td, J=7.6, 4.0 Hz, 1H), 7.13 (d, J=7.7 Hz, 2H), 6.24 (d, J=20.5 Hz, 1H), 5.72 (dq, J=9.9, 5.2 Hz, 0.4H), 5.37 (tt, J=10.6, 3.9 Hz, 0.6H), 4.81 (dd, J=14.6, 6.0 Hz, 1H), 4.68 (d, J=13.3 Hz, 1H), 4.56-4.48 (m, 0.4H), 4.48-4.36 (m, 0.6H), 4.28-4.16 (m, 0.6H), 3.97 (dd, J=14.3, 4.4 Hz, 0.6H), 3.71 (dd, J=14.9, 3.9 Hz, 0.4H), 3.67-3.54 (m, 1H), 3.53-3.32 (m, 2H), 3.27-3.19 (m, 0.4H), 3.08 (dd, J=14.1, 10.9 Hz, 0.6H), 2.53 (d, J=13.5 Hz, 0.6H), 2.46 (d, J=13.7 Hz, 0.4H), 2.27 (d, J=13.7 Hz, 0.41H), 2.12 (s, 6H), 2.03 (d, J=13.5 Hz, 0.6H), 1.98 (s, 1H), 1.90-1.76 (m, 5H), 1.75-1.59 (m, 9H). ESI-MS m/z calc. 655.28284, found 656.5 (M+1)⁺; Retention time: 1.76 minutes; LC method A.

### Example 31: Preparation of Compound 80

### Step 1: (16R)-12-(2,6-Dimethylphenyl)-18-(2,2-dimethylpropanoyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione (Compound 80)

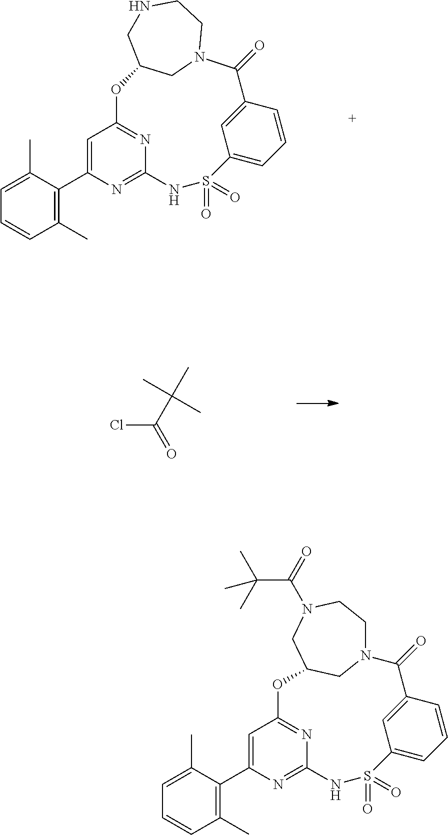

To a stirred solution of (16R)-12-(2,6-dimethylphenyl)-15-oxa-8λ⁶-thia-1,9,11,18,22-pentaazatetracyclo[14.4.1.13,7.110,14]tricosa-3,5,7(23),10,12,14(22)-hexaene-2,8,8-trione (hydrochloride salt) (12 mg, 0.02326 mmol) and triethylamine (15 μL, 0.1076 mmol) in anhydrous dichloromethane (0.6 mL) was added pivaloyl chloride (4 mg, 0.03317 mmol) at 0-5° C. (ice-water bath) under nitrogen. The reaction was stirred at ambient temperature for 3 hours then concentrated under reduced pressure and the crude material was taken up in DMSO (1.0 mL), microfiltered, and purified by preparative reverse-phase HPLC eluting with 1-99% acetonitrile in water over 15 min (HCl as modifier). The desired product (16R)-12-(2,6-dimethylphenyl)-18-(2,2-dimethylpropanoyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione (10.9 mg, 83%) was obtained as white solid. ¹H NMR (400 MHz, Methanol-d₄) δ 8.48-8.36 (m, 1H), 8.01 (ddd, J=5.9, 2.8, 1.7 Hz, 1H), 7.72-7.63 (m, 2H), 7.26 (t, J=7.6 Hz, 1H), 7.14 (d, J 7.6 Hz, 2H), 6.25 (broad s, 1H), 5.57 (broad s, 1H), 4.69 (s, 1H), 4.53 (d, J=13.8 Hz, 1H), 4.41 (dt, J=14.3, 7.4 Hz, 1H), 3.80 (d, J=14.1 Hz, 1H), 3.64-3.49 (m, 2H), 3.38-3.32 (m, 1H), 3.27-3.14 (m, 1H), 2.11 (s, 6H), 1.35 (s, 9H). ESI-MS m/z calc. 563.2202, found 564.4 (M+1)⁺; Retention time: 1.35 minutes; LC method A.

### Example 32: Preparation of Compound 81

### Step 1: (16R)-18-(3,3-Dimethylbutanoyl)-12-(2,6-dimethylphenyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione (Compound 81)

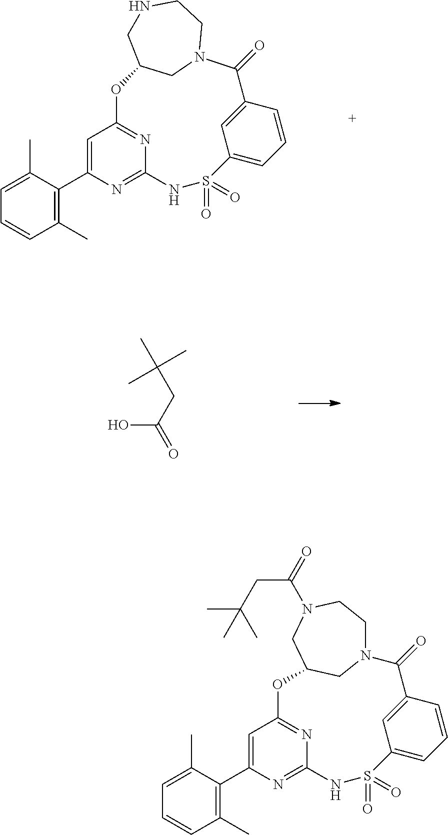

To a stirred solution of (16R)-12-(2,6-dimethylphenyl)-15-oxa-8λ⁶-thia-1,9,11,18,22-pentaazatetracyclo[14.4.1.13,7.110,14]tricosa-3,5,7(23),10,12,14(22)-hexaene-2,8,8-trione (hydrochloride salt) (12 mg, 0.02326 mmol) and N,N-diisopropyl ethyl amine (15 μL, 0.08612 mmol) in anhydrous dichloromethane (0.6 mL) was added 3,3-dimethylbutanoic acid (4 mg, 0.03444 mmol) at 0-5° C. (ice-water bath) under nitrogen, followed by addition of 1-[bis(dimethylamino)methylene]-1H-1,2,3-triazolo[4,5-b]pyridinium 3-oxid hexafluorophosphate (11 mg, 0.02893 mmol) (HATU). The reaction was stirred at ambient temperature for 3 hours then concentrated under reduced pressure. The residue was taken up in DMSO (1.0 mL), microfiltered, and purified by preparative reverse-phase HPLC eluting with 1-99% acetonitrile in water over 15 min (HCl as modifier). The desired product (16R)-18-(3,3-dimethylbutanoyl)-12-(2,6-dimethylphenyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione (8.6 mg, 64%) was obtained as white solid. ¹H NMR (400 MHz, Methanol-d₄) δ 8.47 (dt, J=16.0, 1.5 Hz, 1H), 8.01 (ddt, J=7.6, 3.4, 1.8 Hz, 1H), 7.74-7.63 (m, 2H), 7.27 (td, J=7.7, 3.4 Hz, 1H), 7.22-7.06 (m, 2H), 6.24 (d, J=15.8 Hz, 1H), 5.72 (tt, J=9.6, 4.6 Hz, 0.5H), 5.45 (td, J=10.3, 5.1 Hz, 0.5H), 4.75 (ddd, J=14.6, 6.4, 3.5 Hz, 0.5H), 4.63 (d, J=12.8 Hz, 0.5H), 4.56-4.40 (m, 1H), 4.32 (dt, J=14.5, 5.1 Hz, 0.5H), 4.17 (ddd, J=13.9, 9.7, 6.4 Hz, 0.5H), 3.86 (dd, J=14.2, 4.3 Hz, 0.5H), 3.76-3.69 (m, 0.5H), 3.69-3.62 (m, 1H), 3.59-3.41 (m, 1.5H), 3.41-3.32 (m, 0.5H), 3.23 (dt, J=13.9, 4.5 Hz, 0.5H), 3.12 (dd, J=14.2, 10.9 Hz, 0.5H), 2.52 (d, J=14.3 Hz, 0.5H), 2.51 (s, 1H), 2.30 (d, J=14.3 Hz, 0.5H), 2.12 (s, 6H), 1.11 (s, 4H), 1.09 (s, 5H). (rotamers in about 5:4 ratio) ESI-MS m/z calc. 577.2359, found 578.4 (M+1)⁺; Retention time: 1.46 minutes; LC method A.

### Example 33: Preparation of Compound 82

### Step 1: 04-Benzyl 01-tert-butyl 6-hydroxy-1,4-diazepane-1,4-dicarboxylate

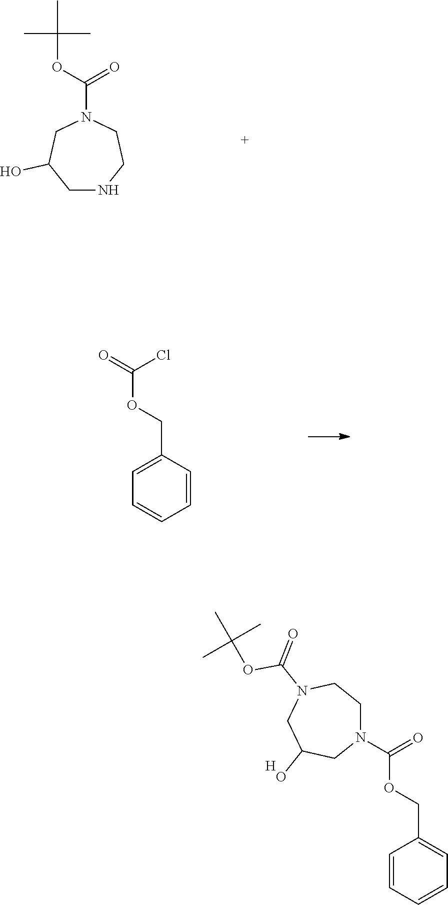

To a solution of tert-butyl 6-hydroxy-1,4-diazepane-1-carboxylate (1.87 g, 8.6463 mmol) in anhydrous dichloromethane (80 mL) cooled to 0° C. was added triethylamine (2.0110 g, 2.77 mL, 19.874 mmol) followed by benzyl chloroformate (1.6252 g, 1.36 mL, 9.5268 mmol). The mixture was stirred at 0° C. for 30 minutes then at room temperature overnight. More benzyl chloroformate (179.25 mg, 150 μL, 1.0507 mmol) was added at room temperature and the mixture was stirred for 3 hours. Ethyl acetate (250 mL) was added then the organic phase was washed with 5% aqueous sodium bicarbonate (4×15 mL) and brine (1×50 mL), dried over anhydrous sodium sulfate, filtered and concentrated under reduced pressure. The crude product was purified by flash chromatography, loaded with DCM, (80 g silica gel, DCM/EtOAc 100:0 to 25:75) to yield 04-benzyl 01-tert-butyl 6-hydroxy-1,4-diazepane-1,4-dicarboxylate (2.15 g, 71%) as a colorless oil. ESI-MS m/z calc. 350.1842, found 373.2 (M+Na)⁺; Retention time: 1.875 minutes (LC method N). ¹H NMR (300 MHz, CDCl₃) δ 1.45-1.47 (m, 9H), 3.02-4.06 (m, 10H), 5.08-5.17 (m, 2H), 7.35-7.36 (m, 5H).

### Step 2: 04-Benzyl 01-tert-butyl 6-oxo-1,4-diazepane-1,4-dicarboxylate

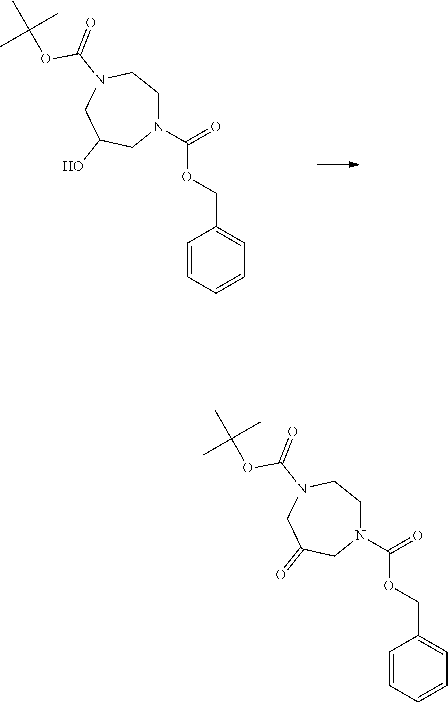 [SMILES: CC(C)(C)OC(=O)N1CCN(C(=O)OCc2ccccc2)CC(O)C1 ; CC(C)(C)OC(=O)N1CCN(C(=O)OCc2ccccc2)CC(=O)C1]

To a solution of 04-benzyl 01-tert-butyl 6-hydroxy-1,4-diazepane-1,4-dicarboxylate (2.03 g, 5.7932 mmol) in anhydrous dichloromethane (65 mL) cooled to 0° C. was added Dess-martin periodinane (3.669 g, 8.6504 mmol). The mixture was stirred from 0° C. to room temperature over 2 hours. The reaction mixture was diluted with ethyl acetate (250 mL). The organic phase was washed with 5% aqueous sodium bicarbonate (4×100 mL) and brine (100 mL), dried over sodium sulfate, filtered and the solvent was removed under reduced pressure. The residue was purified by flash chromatography (loaded in DCM) (80 g silica gel) eluting with mixture of 0-20% ethyl acetate in dichloromethane to afford O4-benzyl O1-tert-butyl 6-oxo-1,4-diazepane-1,4-dicarboxylate (1.668 g, 83%) as a yellow oil. ESI-MS m/z calc. 348.1685, found 371.2 (M+Na)⁺; Retention time: 2.062 minutes (LC method N).

### Step 3: O4-Benzyl O1-tert-butyl 6-hydroxy-6-methyl-1,4-diazepane-1,4-dicarboxylate

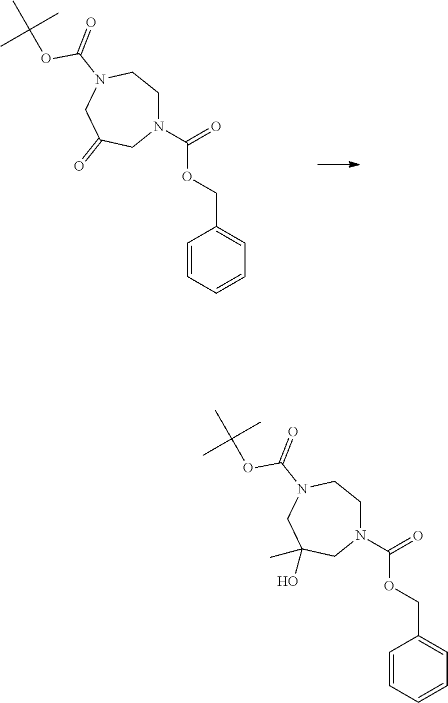 [SMILES: CC(C)(C)OC(=O)N1CCN(C(=O)OCc2ccccc2)CC(=O)C1 ; CC1(O)CN(C(=O)OCc2ccccc2)CCN(C(=O)OC(C)(C)C)C1]

To a solution of O4-benzyl O1-tert-butyl 6-oxo-1,4-diazepane-1,4-dicarboxylate (647 mg, 1.8571 mmol) in dry THE (50 mL) at −20° C. was added dropwise a solution of methylmagnesium bromide (0.760 mL of 3 M in diethyl ether, 2.2800 mmol). The reaction was allowed to warm to room temperature over 2 h, then quenched with a saturated solution of ammonium chloride (50 mL), partitioned between water (150 mL) and EtOAc (200 mL). The aqueous layer was extracted with EtOAc (2×100 mL). The combined organic layers were dried over anhydrous sodium sulfate, filtered, and concentrated under reduced pressure. The crude product was purified by flash chromatography (loaded with DCM, 40 g silica gel, DCM/EtOAc 100:0 to 75:25) to yield 04-benzyl 01-tert-butyl 6-hydroxy-6-methyl-1,4-diazepane-1,4-dicarboxylate (511 mg, 75%) as a pale-yellow oil. ESI-MS m/z calc. 364.1998, found 387.2 (M+Na)⁺; Retention time: 2.8 minutes (LC method H). ¹H NMR (300 MHz, DMSO-d₆) δ 0.96-1.07 (m, 3H), 1.26-1.47 (m, 9H), 2.89-3.28 (m, 4H), 3.50-3.78 (m, 4H), 4.81 (d, J=3.2 Hz, 1H), 4.98-5.20 (m, 2H), 7.20-7.46 (m, 5H).

### Step 4: tert-Butyl 6-hydroxy-6-methyl-1,4-diazepane-1-carboxylate

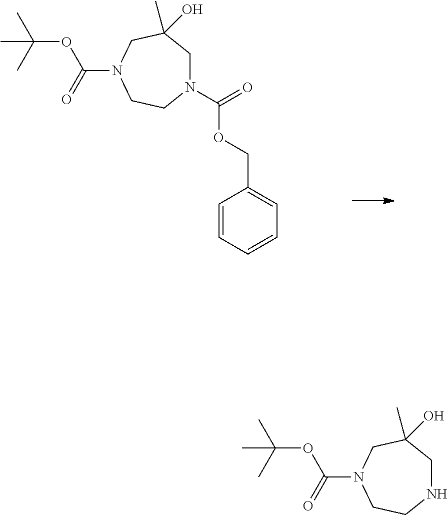 [SMILES: CC1(O)CN(C(=O)OCc2ccccc2)CCN(C(=O)OC(C)(C)C)C1 ; CC1(O)CNCCN(C(=O)OC(C)(C)C)C1]

To a solution of O4-benzyl O1-tert-butyl 6-hydroxy-6-methyl-1,4-diazepane-1,4-dicarboxylate (100 mg, 0.2744 mmol) in methanol (7 mL) was added palladium on carbon (10 mg, 10% w/w, 0.0094 mmol) and the mixture was stirred under hydrogen at 1 atm overnight. Then it was filtered through Celite and the filtrate was evaporated to give crude tert-butyl 6-hydroxy-6-methyl-1,4-diazepane-1-carboxylate (59.4 mg, 89%) as a colorless oil. ESI-MS m/z calc. 230.16304, found 231.1 (M+1)⁺; Retention time: 1.51 minutes; LC method T.

### Step 5: tert-Butyl 4-benzyl-6-hydroxy-6-methyl-1,4-diazepane-1-carboxylate

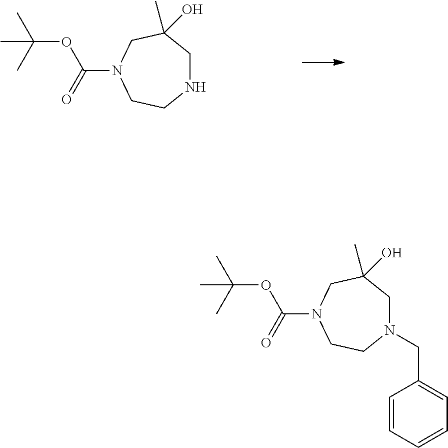 [SMILES: CC1(O)CNCCN(C(=O)OC(C)(C)C)C1 ; CC1(O)CN(Cc2ccccc2)CCN(C(=O)OC(C)(C)C)C1]

To a solution of tert-butyl 6-hydroxy-6-methyl-1,4-diazepane-1-carboxylate (62.4 mg, 0.2709 mmol) and benzaldehyde (30.276 mg, 0.029 mL, 0.2853 mmol) in anhydrous DCM (2 mL) was added sodium triacetoxyborohydride (65.4 mg, 0.3086 mmol). The resulting solution was stirred at ambient temperature for 2 hours, then additional sodium triacetoxyborohydride (33.6 mg, 0.1585 mmol) was added and stirred for another 2 hours. Reaction solution was partitioned between a saturated sodium bicarbonate aqueous solution (15 mL) and dichloromethane (80 mL). The organic layer was separated, dried over sodium sulfate, filtered and concentrated under reduced pressure. The crude product was purified flash chromatography (loaded in DCM) (25 g silica gel, eluting 0 to 30% EtOAc/hexanes) to afford tert-butyl 4-benzyl-6-hydroxy-6-methyl-1,4-diazepane-1-carboxylate (72.3 mg, 81%) as a pale-yellow liquid. ESI-MS m/z calc. 320.21, found 321.2 (M+1)⁺; Retention time: 3.04 minutes; LC method S.

### Step 6: 1-Benzyl-6-methyl-1,4-diazepan-6-ol

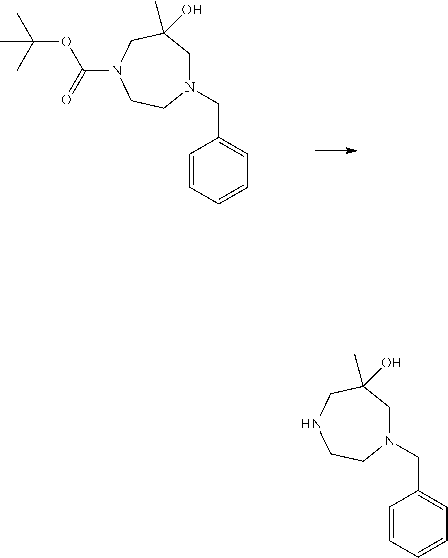 [SMILES: CC1(O)CN(Cc2ccccc2)CCN(C(=O)OC(C)(C)C)C1 ; CC1(O)CNCCN(Cc2ccccc2)C1]

To a solution of tert-butyl 4-benzyl-6-hydroxy-6-methyl-1,4-diazepane-1-carboxylate (72.3 mg, 0.2193 mmol) in dioxane (1.5 mL) was added HCl (0.5 mL of 4 M in dioxane, 2.0000 mmol). The resulting solution was stirred at ambient temperature for 16 hours. All solvents were removed under reduced pressure. The residue was dried under vacuum for 4 hours to afford 1-benzyl-6-methyl-1,4-diazepan-6-ol (dihydrochloride salt) (64.3 mg, 95%) as a sticky solid. This product was used directly in next step without any purification. ESI-MS m/z calc. 220.15756, found 221.4 (M+1)⁺; Retention time: 1.58 minutes; LC method S.

### Step 7: 3-(4-Benzyl-6-hydroxy-6-methyl-1,4-diazepane-1-carbonyl)-N-[4-chloro-6-(2,6-dimethylphenyl)pyrimidin-2-yl]benzenesulfonamide

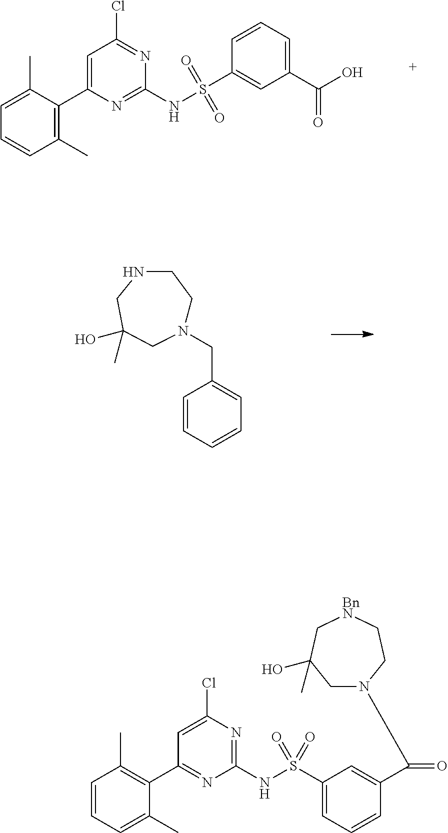 [SMILES: Cc1cccc(C)c1-c1cc(Cl)nc(NS(=O)(=O)c2cccc(C(=O)O)c2)n1 ; CC1(O)CNCCN(Cc2ccccc2)C1 ; Cc1cccc(C)c1-c1cc(Cl)nc(NS(=O)(=O)c2cccc(C(=O)N3CCN(Cc4ccccc4)CC(C)(O)C3)c2)n1]

To a suspension solution of 3-[[4-chloro-6-(2,6-dimethylphenyl)pyrimidin-2-yl]sulfamoyl]benzoic acid (91.8 mg, 0.2197 mmol) in anhydrous DCM (2 mL) was added SOCl₂ (1.6310 g, 1 mL, 13.709 mmol). The resulting solution was heated at 35° C. for 2 days. All solvents were removed under reduced pressure. To a suspension of 1-benzyl-6-methyl-1,4-diazepan-6-ol (dihydrochloride salt) (64.3 mg, 0.2083 mmol) in anhydrous DCM (1 mL) at 0° C. was added the solution of benzoyl chloride made above in anhydrous DCM (1 mL). The resulting solution was stirred at this temperature for 30 minutes, then the reaction solution was diluted with dichloromethane (100 mL) and saturated sodium bicarbonate aqueous solution (15 mL) was added. The organic layer was separated, dried over sodium sulfate, filtered and concentrated under reduced pressure. The crude product was purified by flash chromatography (loaded in DCM) (40 g silica gel, eluting 0 to 100% acetone/hexanes) to afford 3-(4-benzyl-6-hydroxy-6-methyl-1,4-diazepane-1-carbonyl)-N-[4-chloro-6-(2,6-dimethylphenyl)pyrimidin-2-yl]benzenesulfonamide (136 mg, 98%) as a white solid. ESI-MS m/z calc. 619.202, found 620.3 (M+1)⁺; Retention time: 4.63 minutes; LC method S.

### Step 8: 18-Benzyl-12-(2,6-dimethylphenyl)-16-methyl-15-oxa-8λ⁶-thia-1,9,11,18,22-pentaazatetracyclo[14.4.1.13,7.110,14]tricosa-3(23),4,6,10,12,14(22)-hexaene-2,8,8-trione (Compound 82)

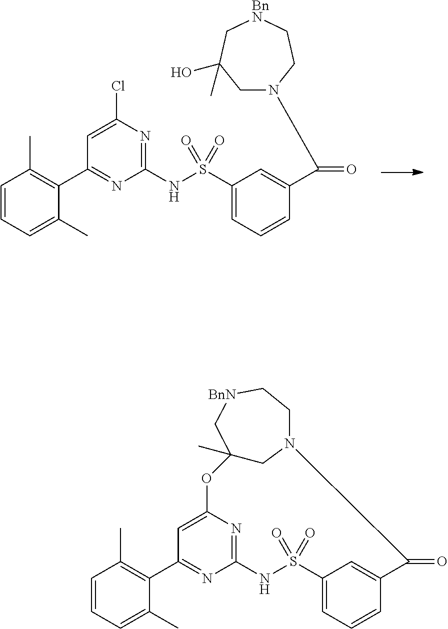 [SMILES: Cc1cccc(C)c1-c1cc(Cl)nc(NS(=O)(=O)c2cccc(C(=O)N3CCN(Cc4ccccc4)CC(C)(O)C3)c2)n1 ; Cc1cccc(C)c1-c1cc2nc(n1)NS(=O)(=O)c1cccc(c1)C(=O)N1CCN(Cc3ccccc3)CC(C)(C1)O2]

To the solution of 3-(4-benzyl-6-hydroxy-6-methyl-1,4-diazepane-1-carbonyl)-N-[4-chloro-6-(2,6-dimethylphenyl)pyrimidin-2-yl]benzenesulfonamide (67 mg, 0.1037 mmol) in anhydrous THE (10 mL) was added NaH (47 mg, 1.1751 mmol). The resulting solution was stirred at ambient temperature for 8 hours and at 50° C. for 16 hours. This reaction was combined with another crude mixture from a reaction run on the same scale and treated with a sodium bicarbonate aqueous solution (20 mL) followed by ethyl acetate (100 mL). The organic layer was separated, washed with brine (2×20 mL), dried over anhydrous sodium sulfate, filtered and concentrated under reduced pressure. The crude product was purified by flash chromatography (loaded in DCM) (40 g silica gel, eluting 0 to 70% acetone/hexanes) and by reverse HPLC (35% to 65% B, mobile phase A=water (0.1% CF₃CO₂H). mobile phase B=acetonitrile (0.1% CF₃CO₂H)). Pure fractions were combined and lyophilized to give a TFA salt, which was dissolved in water (5 mM HCl) and lyophilized again to afford 18-benzyl-12-(2,6-dimethylphenyl)-16-methyl-15-oxa-8λ⁶-thia-1,9,11,18,22-pentaazatetracyclo[14.4.1.13,7.110,14]tricosa-3(23),4,6,10,12,14(22)-hexaene-2,8,8-trione (hydrochloride salt) (13 mg, 9% combined yield) as a white solid. ¹H NMR (250 MHz, DMSO-d6) δ 8.49 (s, 1H), 7.87 (s, 1H), 7.68 (d, J=4.6 Hz, 2H), 7.59-7.30 (m, 5H), 7.30-7.17 (m, 1H), 7.11 (d, J=7.6 Hz, 2H), 6.23 (s, 1H), 4.54 (d, J=15.0 Hz, 2H), 4.0-3.71 (m, 4H, overlap with water), 3.52 (d, J=15.2 Hz, 2H), 3.35-2.92 (m, 2H), 2.22 (s, 3H), 2.04 (s, 6H). ESI-MS m/z calc. 583.22534, found 584.2 (M+1)⁺; Retention time: 1.96 minutes; LC method T.

### Example 34: Preparation of Compound 83 and Compound 84

### Step 1: Methyl (2R)-2-(tert-butoxycarbonylamino)-4-methyl-pentanoate

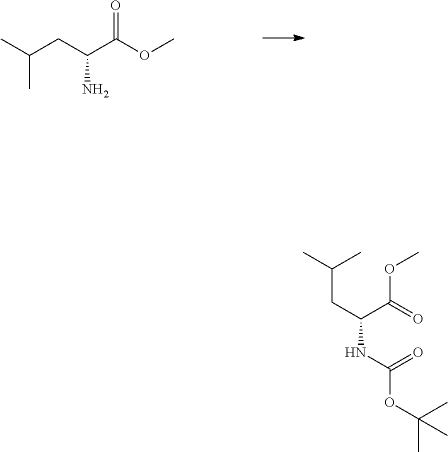

To a solution of methyl (2R)-2-amino-4-methyl-pentanoate (hydrochloride salt) (50 g, 269.74 mmol) in a mixture of dioxane (313 mL) and aqueous sodium bicarbonate (1250 mL) at 0° C. was added Boc anhydride (61.814 g, 283.23 mmol). The resulting solution was stirred for 20 hours while it warmed up to ambient temperature. The reaction solution was extracted with ethyl acetate (3×500 mL). The combined organic layers were washed with brine (200 mL), dried over magnesium sulfate, filtered and concentrated under reduced pressure to afford methyl (2R)-2-(tert-butoxycarbonylamino)-4-methyl-pentanoate (64.13 g, 92%) as a pale-yellow oil. ¹H NMR (250 MHz, CDCl₃) δ 4.88 (m, 1H), 4.32 (m, 1H), 3.79-3.67 (m, 3H), 1.76-1.58 (m, 2H), 1.58-1.40 (m, 10H), 0.95 (d, J 2.3 Hz, 3H), 0.93 (d, J 2.3 Hz, 3H). ESI-MS m/z calc. 245.1627, found 246.1 (M+1)⁺; Retention time: 4.97 minutes; LC method S.

### Step 2: tert-Butyl N-[(1R)-1-(2-chloroacetyl)-3-methyl-butyl]carbamate

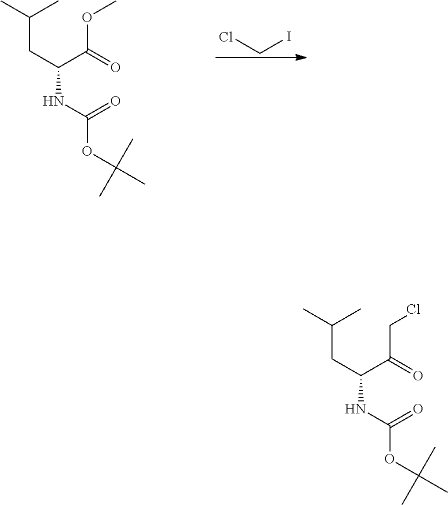

To a solution of diisopropyl amine (52.313 g, 72.456 mL, 516.98 mmol) in anhydrous THE (350 mL) at −50° C. was added n-BuLi (207.03 mL of 2.5 M, 517.57 mmol) slowly, then the reaction was warmed up to 0° C. and stirred for an additional 30 minutes before it was cooled to −78° C. The resulting LDA solution was added dropwise via canula to a precooled solution of methyl (2R)-2-(tert-butoxycarbonylamino)-4-methyl-pentanoate (25 g, 96.814 mmol) and chloro(iodo)methane (69.9 g, 388.37 mmol) in anhydrous THE (650 mL) at −78° C. over 35 minutes. After the addition was finished, the resulting solution was further stirred at this temperature for 1 hour. A mixture of acetic acid (82 mL) and THE (82 mL) was added slowly to quench the reaction. The reaction solution was warmed up to 0° C., then water (500 mL) was added and the organic layer was separated. The aqueous layer was extracted with ethyl acetate (2×300 mL). The combined organic layers were washed with brine (200 mL), dried over sodium sulfate, filtered and concentrated under reduced pressure. The crude product was purified by flash chromatography (loaded in DCM) (330 g silica gel, eluting 0 to 15% EtOAc/hexanes) to afford tert-butyl N-[(1R)-1-(2-chloroacetyl)-3-methyl-butyl]carbamate (19.19 g, 75%) as a pale-yellow solid. ¹H NMR (250 MHz, CDCl₃) δ 5.16-4.75 (m, 1H), 4.64-4.41 (m, 1H), 4.29 (d, J=3.0 Hz, 2H), 1.91-1.48 (m, 3H), 1.44 (s, 9H), 0.97 (d, J=4.2 Hz, 3H), 0.93 (d, J=4.2 Hz, 3H).

### Step 3: tert-Butyl 2-(benzylamino)acetate

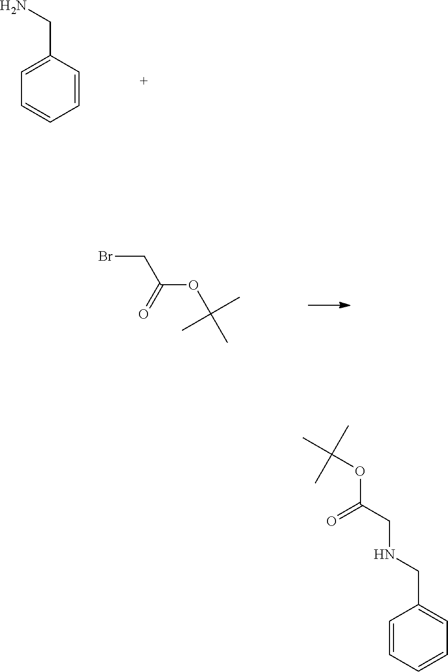

tert-butyl 2-bromoacetate (30 mL, 194.98 mmol) was added dropwise to a solution of phenylmethanamine (85 mL, 770.42 mmol) in toluene (150 mL) then the mixture was heated at 72° C. for 1 hour. The mixture was poured in a 1 N sodium hydroxide solution (200 mL) and extracted with ethyl acetate (2×200 mL). The organic phases were combined, washed with water (200 mL) and brine (200 mL), dried over anhydrous sodium sulfate, filtered and concentrated under reduced pressure. The residue was purified by silica-gel chromatography on a 330 g column, eluting from 0% to 40% of ethyl acetate in hexanes to afford tert-butyl 2-(benzylamino)acetate (39.9 g, 90%). ESI-MS m/z calc. 221.14159, found 222.6 (M+1)⁺; Retention time: 1.93 minutes; LC method T.

### Step 4: tert-Butyl 2-[benzyl-[(3R)-3-(tert-butoxycarbonylamino)-5-methyl-2-oxo-hexyl]amino]acetate

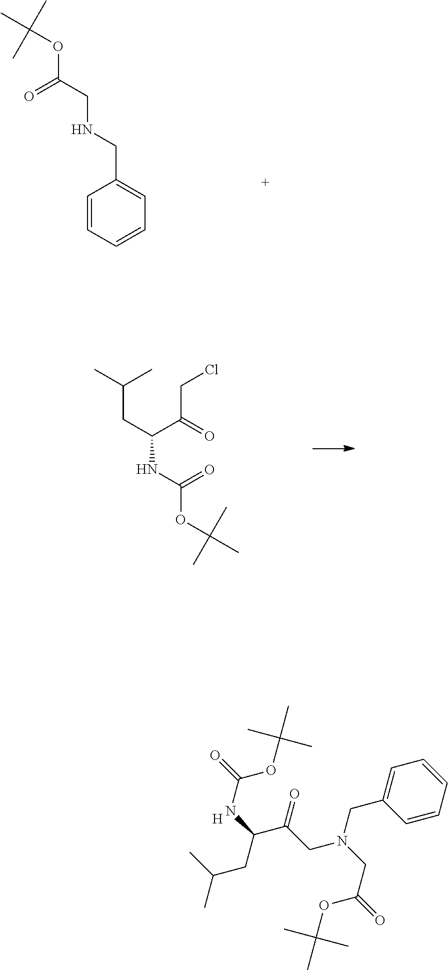

To a solution of tert-butyl 2-(benzylamino)acetate (12.04 g, 51.687 mmol) and tert-butyl N-[(1R)-1-(2-chloroacetyl)-3-methyl-butyl]carbamate (15.725 g, 56.637 mmol) in anhydrous DMF (110 mL) was added sodium bicarbonate (11.6 g, 138.08 mmol) followed by sodium iodide (5.08 g, 33.891 mmol). The resulting solution was stirred at ambient temperature for 17 hours before water (400 mL) was added. The solution was extracted with ether (2×400 mL). The combined organic layers were washed with brine (200 mL), dried over sodium sulfate, filtered and concentrated in vacuo to afford crude product. The crude product was purified by flash chromatography (loaded in DCM) (330 g silica gel, eluting 0 to 15% EtOAc/hexanes) to afford tert-butyl 2-[benzyl-[(3R)-3-(tert-butoxycarbonylamino)-5-methyl-2-oxo-hexyl]amino]acetate (22.85 g, 85%) as a pale-yellow liquid. ¹H NMR (250 MHz, CDCl₃) δ 7.66-6.79 (m, 5H), 5.12-4.85 (m, 1H), 4.40 (s, 1H), 3.96-3.76 (m, 2H), 3.76-3.54 (m, 2H), 3.36 (d, J=1.5 Hz, 2H), 1.91-1.52 (m, 2H), 1.52-1.33 (m, 18H), 1.31-1.23 (m, 1H), 0.94 (d, J=6.5 Hz, 3H), 0.88 (d, J=6.5 Hz, 3H). ESI-MS m/z calc. 448.29373, found 449.2 (M+1)⁺; Retention time: 5.04 minutes; LC method S.

### Step 5: tert-Butyl 2-[benzyl-[(2S,3R)-3-(tert-butoxycarbonylamino)-2-hydroxy-5-methyl-hexyl]amino]acetate and tert-butyl 2-[benzyl-[(2R,3R)-3-(tert-butoxycarbonylamino)-2-hydroxy-5-methyl-hexyl]amino]acetate

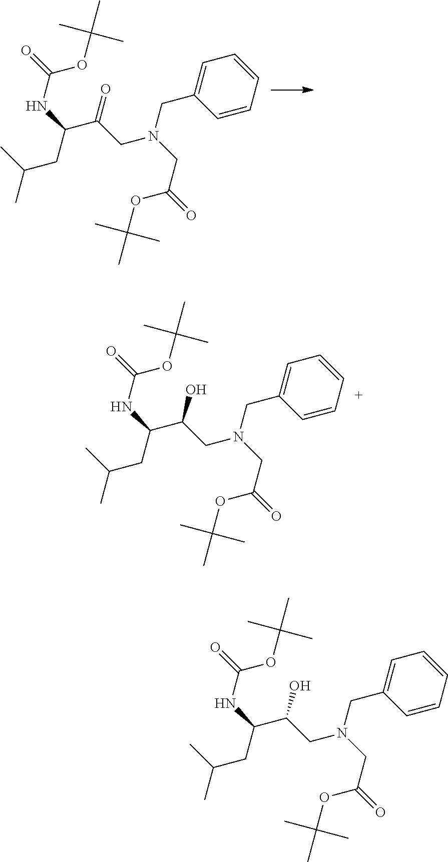 [SMILES: CC(C)C[C@@H](NC(=O)OC(C)(C)C)C(=O)CN(CC(=O)OC(C)(C)C)Cc1ccccc1 ; CC(C)C[C@@H](NC(=O)OC(C)(C)C)[C@@H](O)CN(CC(=O)OC(C)(C)C)Cc1ccccc1 ; CC(C)C[C@@H](NC(=O)OC(C)(C)C)[C@H](O)CN(CC(=O)OC(C)(C)C)Cc1ccccc1]

To a solution of tert-butyl 2-[benzyl-[(3R)-3-(tert-butoxycarbonylamino)-5-methyl-2-oxo-hexyl]amino]acetate (21.93 g, 48.886 mmol) in MeOH (220 mL) at 0° C. was added sodium borohydride (3.745 g, 98.989 mmol) (internal temperature<24° C.). The reaction solution was stirred at 0° C. for 20 minutes. Water (250 mL) was added. The solution was extracted with ethyl acetate (2×400 mL). The combined organic layers were dried over sodium sulfate, filtered and concentrated in vacuo. Benzene (2×100 mL) was added and concentrated in vacuo to remove water. The crude product was purified by flash chromatography (loaded in DCM) (330 g silica gel, eluting 0 to 20% EtOAc/hexanes) to afford tert-butyl 2-[benzyl-[(2S,3R)-3-(tert-butoxycarbonylamino)-2-hydroxy-5-methyl-hexyl]amino]acetate (15.13 g, 65%) (more polar isomer) as a pale-yellow oil. ESI-MS m/z calc. 450.3094, found 451.2 (M+1)⁺; Retention time: 4.74 minutes (LC method S); ¹H NMR (250 MHz, CDCl₃) δ 7.51-7.06 (m, 5H), 4.63 (d, J=9.2 Hz, 1H), 3.87 (d, J=13.5 Hz, 1H), 3.69 (d, J=13.5 Hz, 1H), 3.64-3.46 (m, 2H), 3.19 (d, J=1.8 Hz, 2H), 2.84 (d, J=13.1 Hz, 1H), 2.54 (dd, J=13.0, 9.8 Hz, 1H), 1.78-1.54 (m, 1H), 1.44 (s, 9H), 1.43 (s, 9H), 1.42-1.27 (m, 2H), 0.91 (d, J=4.3 Hz, 3H), 0.89 (d, J=4.3 Hz, 3H); and tert-butyl 2-[benzyl-[(2R,3R)-3-(tert-butoxycarbonylamino)-2-hydroxy-5-methyl-hexyl]amino]acetate (4.36 g, 19%) (less polar isomer) as a pale-yellow oil, ESI-MS m/z calc. 450.3094, found 451.2 (M+1)⁺; Retention time: 4.73 minutes (LC method S); ¹H NMR (250 MHz, CDCl₃) δ 7.48-7.04 (m, 5H), 4.76 (d, J=9.9 Hz, 1H), 3.89 (d, J=13.5 Hz, 1H), 3.77-3.32 (m, 4H), 3.19 (d, J=5.2 Hz, 2H), 2.73 (dd, J=13.1, 3.2 Hz, 1H), 2.52 (dd, J=13.1, 10.7 Hz, 1H), 1.78-1.49 (m, 3H), 1.44 (s, 9H), 1.40 (s, 9H), 1.07-0.69 (m, 6H).

### Step 6: (6R,7R)-4-Benzyl-6-hydroxy-7-isobutyl-1,4-diazepan-2-one

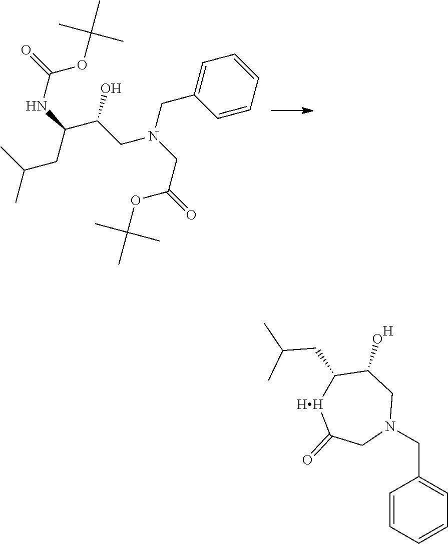

To tert-butyl 2-[benzyl-[(2R,3R)-3-(tert-butoxycarbonylamino)-2-hydroxy-5-methyl-hexyl]amino]acetate (4.36 g, 9.6758 mmol) was added HCl (100 mL of 4 M in dioxane, 400.00 mmol). The resulting solution was stirred at ambient temperature for 24 hours. Then the solvents were removed under reduced pressure. The residue was dissolved in anhydrous EtOH (200 mL). The resulting solution was stirred at 50° C. for 6 hours. Then TEA (9.8010 g, 13.5 mL, 96.857 mmol) was added and the reaction continued for 15 hours at 50° C. All solvents were removed under reduced pressure. The residue was dissolved in ethyl acetate (500 mL) and washed with saturated sodium bicarbonate aqueous solution (100 mL). The organic layer was separated, and the aqueous layer was extracted with ethyl acetate (100 mL). The combined organic layers were dried over sodium sulfate, filtered and concentrated under reduced pressure. The crude product was purified by flash chromatography (loaded in DCM) (120 g silica gel, eluting 0 to 100% EtOAc/hexanes) to afford (6R,7R)-4-benzyl-6-hydroxy-7-isobutyl-1,4-diazepan-2-one (2.19 g, 81%) as a white foam solid. ESI-MS m/z calc. 276.18378, found 277.1 (M+1)⁺; Retention time: 2.08 minutes; LC method S.

### Step 7: tert-Butyl (6R,7R)-4-benzyl-6-hydroxy-7-isobutyl-1,4-diazepane-1-carboxylate

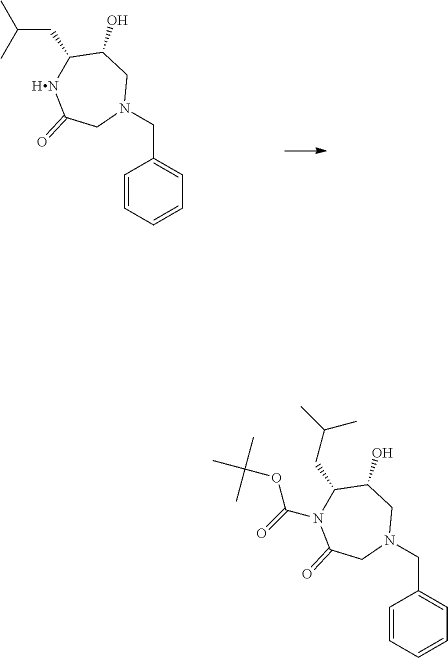

To a solution of (6R,7R)-4-benzyl-6-hydroxy-7-isobutyl-1,4-diazepan-2-one (2.19 g, 7.5595 mmol) in anhydrous THE (76 mL) was added LAH (1.72 g, 45.318 mmol) very slowly. The suspension solution was heated at 40° C. under argon for 16 hours. The reaction solution was cooled to 0° C., then water (1.7 mL) was added dropwise followed by the addition of 15% of NaOH aqueous solution (1.7 mL) and water (5.1 mL). THE (80 mL) was added and the suspension was stirred at ambient temperature for 1 hour. The suspension was filtered through Celite and washed with THE (100 mL). The filtrate was concentrated under reduced pressure to afford crude amino alcohol intermediate as a colorless liquid which was dissolved in a mixture of dioxane (40 mL) and a sodium bicarbonate saturated aqueous solution (40 mL). Boc anhydride (2.09 g, 9.5763 mmol) was added and the resulting solution was stirred at ambient temperature for 16 hours. Then additional Boc anhydride (0.43 g, 1.9702 mmol) was added and the reaction was stirred for an additional 8 hour. Water (50 mL) and ethyl acetate (100 mL) were added. The organic layer was separated, and the aqueous layer was extracted with ethyl acetate (2×100 mL). The combined organic layers were dried over anhydrous sodium sulfate, filtered and concentrated under reduced pressure. The crude product was purified by flash chromatography (loaded in hexane) (120 g silica gel, eluting 0 to 15% EtOAc/hexanes) to afford tert-butyl (6R,7R)-4-benzyl-6-hydroxy-7-isobutyl-1,4-diazepane-1-carboxylate (1.9399 g, 65%) as a colorless liquid. ¹H NMR (250 MHz, DMSO-d₆) δ 7.48-7.09 (m, 5H), 4.35 (s, 1H), 4.13-3.74 (m, 2H), 3.65 (t, J=2.6 Hz, 2H), 3.52 (d, J=15.1 Hz, 1H), 3.14-2.75 (m, 2H), 2.68 (d, J=12.3 Hz, 1H), 2.46-2.10 (m, 2H), 1.69-1.44 (m, 2H), 1.43-1.21 (m, 10H), 0.95-0.77 (m, 6H). ESI-MS m/z calc. 362.25696, found 363.6 (M+1)⁺; Retention time: 1.86 minutes; LC method T.

### Step 8: (5R,6R)-1-Benzyl-5-isobutyl-1,4-diazepan-6-ol

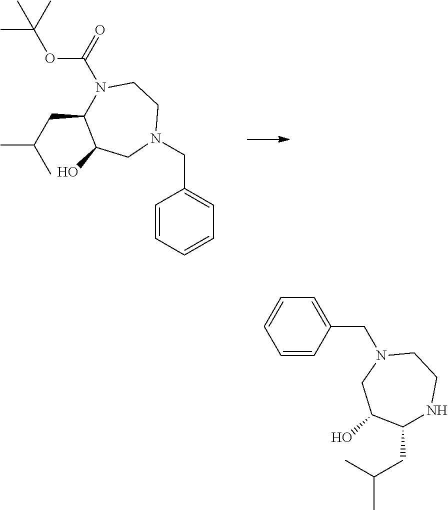 [SMILES: CC(C)C[C@@H]1[C@H](O)CN(Cc2ccccc2)CCN1C(=O)OC(C)(C)C ; CC(C)C[C@H]1NCCN(Cc2ccccc2)C[C@H]1O]

A 100 mL round bottom flask was charged with tert-butyl (6R,7R)-4-benzyl-6-hydroxy-7-isobutyl-1,4-diazepane-1-carboxylate (465 mg, 1.283 mmol), dioxane (4 mL) and HCl (4 mL of 4 M, 16.00 mmol) (4M dioxane solution). The mixture was stirred at rt for 2 hours (about 30% conversion by LCMS). More HCl (4 mL of 4 M, 16.00 mmol) was added and the solution was stirred at rt for 3 hours. The volatiles were removed under reduced pressure. The residue was treated with diethylether, DCM and hexanes and the solvents were evaporated. The operation was repeated until a solid was obtained. Drying in vacuo provided (5R,6R)-1-benzyl-5-isobutyl-1,4-diazepan-6-ol (Dihydrochloride salt) (492 mg, 100%) as a grey solid. ¹H NMR (400 MHz, DMSO-d₆+10% D₂O) δ 7.59-7.46 (m, 5H), 4.47-4.29 (m, 2H), 3.75-3.46 (m, 6H), 1.69-1.51 (m, 2H), 1.51-1.34 (m, 1H), 0.97-0.82 (m, 6H). ESI-MS m/z calc. 262.2045, found 263.26 (M+1)⁺; Retention time: 0.48 minutes, LC method A.

### Step 9: 3-[(6R,7R)-4-Benzyl-6-hydroxy-7-isobutyl-1,4-diazepane-1-carbonyl]-N-[4-chloro-6-(2,6-dimethylphenyl)pyrimidin-2-yl]benzenesulfonamide

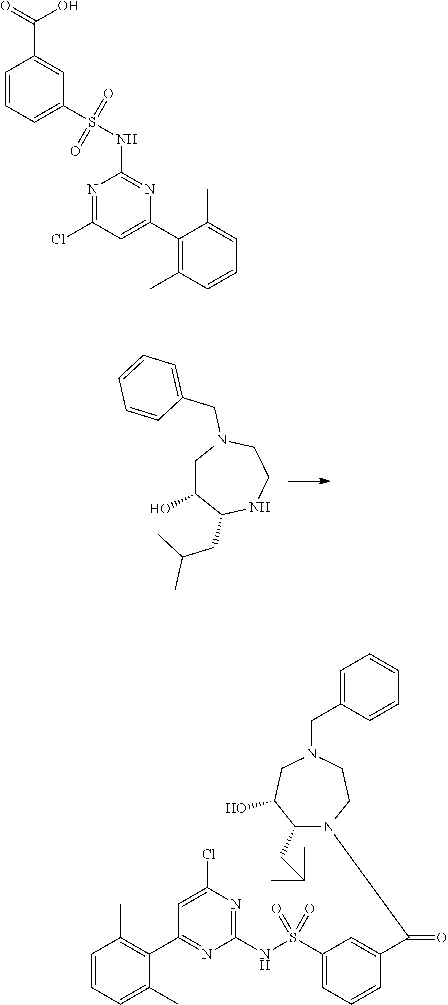 [SMILES: Cc1cccc(C)c1-c1cc(Cl)nc(NS(=O)(=O)c2cccc(C(=O)O)c2)n1 ; CC(C)C[C@H]1NCCN(Cc2ccccc2)C[C@H]1O ; Cc1cccc(C)c1-c1cc(Cl)nc(NS(=O)(=O)c2cccc(C(=O)N3CCN(Cc4ccccc4)C[C@@H](O)[C@H]3CC(C)C)c2)n1]

A 100 mL flask was charged under nitrogen with (5R,6S)-1-benzyl-5-isobutyl-1,4-diazepan-6-ol (Dihydrochloride salt) (492 mg, 1.282 mmol), anhydrous DMF (9 mL) and 3-[[4-chloro-6-(2,6-dimethylphenyl)pyrimidin-2-yl]sulfamoyl]benzoic acid (535 mg, 1.280 mmol). After dissolution of the reagents, the mixture was cooled down in an ice bath. DIEA (1.5 mL, 8.612 mmol) and HATU (590 mg, 1.552 mmol) were added and the mixture was stirred at 0° C. for 2 hours. The reaction mixture was poured in citric acid (50 mL of 10% w/v, 26.02 mmol)(10% aqueous) cooled in ice. The resulting white solid was filtered and washed with water. The solid (1.03 g) was purified by flash chromatography on silica gel (80 g column) using a gradient of methanol (0 to 5% over 30 min) in dichloromethane. The product eluted around 2% methanol to give 3-[(6R,7R)-4-benzyl-6-hydroxy-7-isobutyl-1,4-diazepane-1-carbonyl]-N-[4-chloro-6-(2,6-dimethylphenyl)pyrimidin-2-yl]benzenesulfonamide (275 mg, 32%) as a white solid. ESI-MS m/z calc. 661.24896, found 662.43 (M+1)⁺; Retention time: 1.45 minutes; LC method A.

### Step 10: (16R,21R)-18-Benzyl-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione (Compound 84)

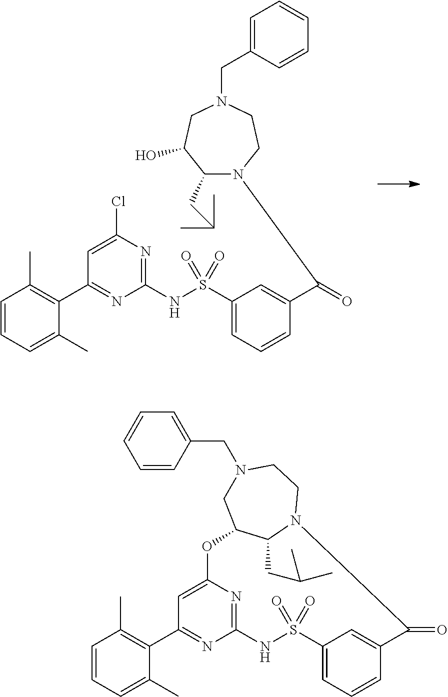 [SMILES: Cc1cccc(C)c1-c1cc(Cl)nc(NS(=O)(=O)c2cccc(C(=O)N3CCN(Cc4ccccc4)C[C@@H](O)[C@H]3CC(C)C)c2)n1 ; Cc1cccc(C)c1-c1cc2nc(n1)NS(=O)(=O)c1cccc(c1)C(=O)N1CCN(Cc3ccccc3)C[C@@H](O2)[C@H]1CC(C)C]

A 100 mL flask was charged under nitrogen with 3-[(6S,7R)-4-benzyl-6-hydroxy-7-isobutyl-1,4-diazepane-1-carbonyl]-N-[4-chloro-6-(2,6-dimethylphenyl)pyrimidin-2-yl]benzenesulfonamide (275 mg, 0.4153 mmol) and anhydrous DMF (25 mL). To the solution was added NaH (133 mg of 60% w/w, 3.325 mmol) and the mixture was stirred under nitrogen at room temperature for 3 days. A 10% aqueous solution of citric acid (100 mL of 10% w/v, 52.05 mmol) was cooled down in ice and the DMF reaction mixture was poured in the cold citric acid solution under stirring. A bit of brine was added, and the resulting solid suspension was extracted with EtOAc (3×30 mL). The first extraction formed an emulsion caused by the presence of a very fine solid that was removed by filtration. After drying over sodium sulfate, evaporation of the solvents gave a residue that was dissolved in DCM containing a bit of methanol (and purified by flash chromatography on silica gel (24 g column) using a gradient of methanol (0 to 10% over 50 min.) in dichloromethane. The product eluted around 3-5% methanol as multiple broad peaks. All the fractions containing the product were combined and the solvents evaporated to give a residue that was turned into an off-white solid after several run of trituration/evaporation in DCM/hexanes. (16R,21R)-18-benzyl-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione (93 mg, 35%). ESI-MS m/z calc. 625.2723, found 626.53 (M+1)⁺; Retention time: 1.34 minutes; LC method A.

### Step 11: (16R,21R)-12-(2,6-Dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione

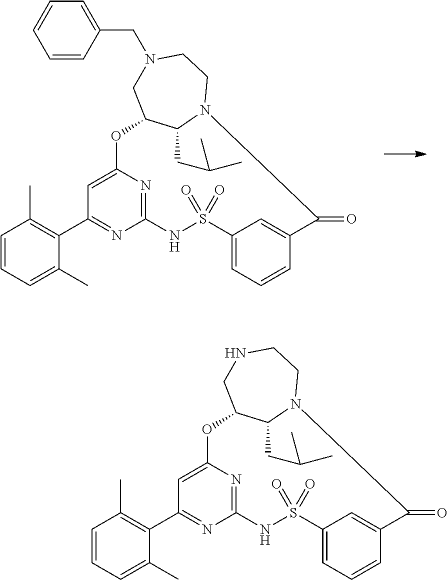 [SMILES: Cc1cccc(C)c1-c1cc2nc(n1)NS(=O)(=O)c1cccc(c1)C(=O)N1CCN(Cc3ccccc3)C[C@@H](O2)[C@H]1CC(C)C ; Cc1cccc(C)c1-c1cc2nc(n1)NS(=O)(=O)c1cccc(c1)C(=O)N1CCNC[C@@H](O2)[C@H]1CC(C)C]

A 100 mL flask was charged with (21R)-18-benzyl-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (85 mg, 0.1331 mmol) and MeOH (20 mL). The solution was sparged with nitrogen. Pd(OH)₂ (45 mg of 20% w/w, 0.06409 mmol) was added and the solution was stirred under an atmosphere of hydrogen (balloon) for 17 hours. The solution was purged with nitrogen. The catalyst was removed by filtration through Celite and the filtrate was concentrated. The residue was dissolved in DCM/MeOH and the solution was filtered. Evaporation of the solvent gave (16R,21R)-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (70 mg, 98%) as an off-white solid. ESI-MS m/z calc. 535.22534, found 536.49 (M+1)⁺; Retention time: 1.07 minutes; LC method A.

### Step 12: (16R,21R)-12-(2,6-Dimethylphenyl)-21-(2-methylpropyl)-18-{spiro[3.4]octan-2-yl}-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 83)

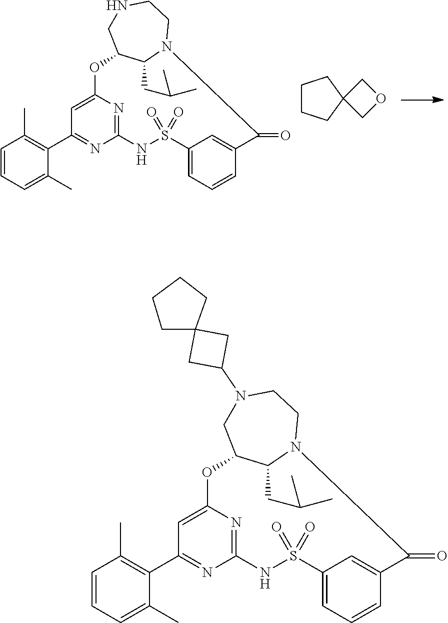

A 4 mL vial was charged with (21R)-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (15 mg, 0.02800 mmol), anhydrous DCM (400 μL), spiro[3.4]octan-2-one (20 mg, 0.1611 mmol) and acetic acid (12 μL, 0.2110 mmol). The vial was briefly purged with nitrogen, capped and stirred at room temperature for about 10 minutes. Sodium triacetoxy borohydride (20 mg, 0.09437 mmol) was added. The vial was purged with nitrogen, capped and the reaction was stirred at room temperature for 14 hours. Methanol (25 μL) was added. The DCM phase was evaporated and the residue was taken in DMSO (1 mL). The solution was microfiltered and purified by reverse phase preparative HPLC (Cis) using a gradient of acetonitrile in water (1 to 99% over 15 min) and HCl as a modifier to give (16R,21R)-12-(2,6-dimethylphenyl)-21-(2-methylpropyl)-18-{spiro[3.4]octan-2-yl}-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (9.7 mg, 50%) as a white solid. ESI-MS m/z calc. 643.3192, found 644.6 (M+1)⁺; Retention time: 1.4 minutes; LC method A.

### Example 35: Preparation of Compound 85 and Compound 86

### Step 1: (6S,7R)-4-Benzyl-6-hydroxy-7-isobutyl-1,4-diazepan-2-one

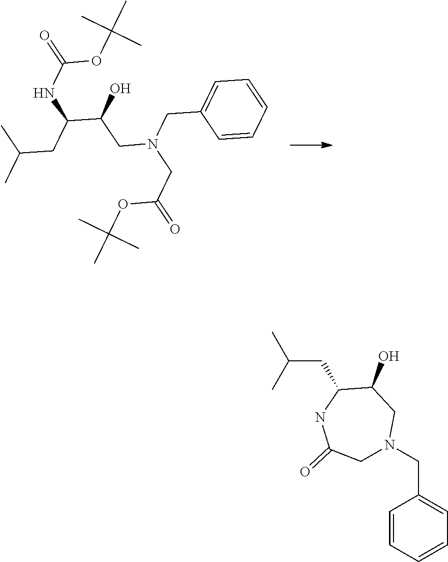 [SMILES: CC(C)C[C@@H](NC(=O)OC(C)(C)C)[C@@H](O)CN(CC(=O)OC(C)(C)C)Cc1ccccc1 ; CC(C)C[C@H]1NC(=O)CN(Cc2ccccc2)C[C@@H]1O]

tert-Butyl 2-[benzyl-[(2S,3R)-3-(tert-butoxycarbonylamino)-2-hydroxy-5-methyl-hexyl]amino]acetate (15.13 g, 33.577 mmol) was added HCl (335 mL of 4 M, 1.3400 mol). The resulting solution was stirred at ambient temperature for 24 hours. Then all solvents were removed under reduced pressure. The residue was dissolved in anhydrous EtOH (700 mL). The resulting solution was stirred at 50° C. for 21 hours. Then TEA (33.977 g, 46.800 mL, 335.77 mmol) was added and the reaction was continued to stir for 7.5 hours at 50° C. All solvents were removed under reduced pressure. The residue was dissolved in ethyl acetate (800 mL) and washed with a saturated sodium bicarbonate aqueous solution (200 mL). The organic layer was separated, and aqueous layer was extracted with ethyl acetate (200 mL). The combined organic layers were dried over sodium sulfate, filtered and concentrated under reduced pressure. The crude product was purified by flash chromatography (loaded in DCM) (220 g silica gel, eluting 0 to 100% EtOAc/hexanes) to afford (6S,7R)-4-benzyl-6-hydroxy-7-isobutyl-1,4-diazepan-2-one (7.33 g, 77%) as a white solid. ESI-MS m/z calc. 276.18378, found 277.2 (M+1)⁺; Retention time: 2.17 minutes; LC method S.

### Step 2: tert-Butyl (6S,7R)-4-benzyl-6-hydroxy-7-isobutyl-1,4-diazepane-1-carboxylate

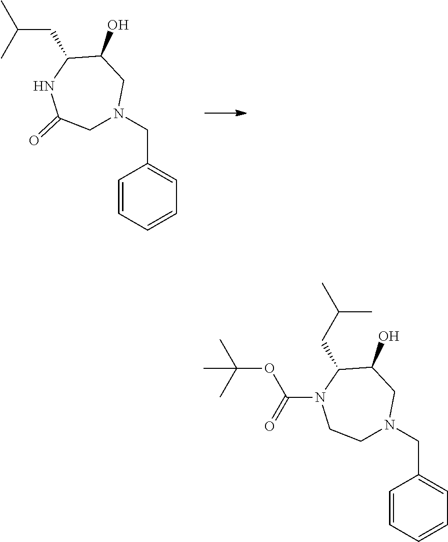 [SMILES: CC(C)C[C@H]1NC(=O)CN(Cc2ccccc2)C[C@@H]1O ; CC(C)C[C@@H]1[C@@H](O)CN(Cc2ccccc2)CCN1C(=O)OC(C)(C)C]

To a solution of (6S,7R)-4-benzyl-6-hydroxy-7-isobutyl-1,4-diazepan-2-one (7.33 g, 25.673 mmol) in anhydrous THF (260 mL) was added LAH (5.86 g, 154.40 mmol) very slowly. The suspension solution was heated at 40° C. under argon for 16 hours. The reaction solution was cooled to 0° C., then water (5.9 mL) was added dropwise followed by the addition of 15% of NaOH aqueous solution (5.9 mL) and water (17.7 mL). THE (200 mL) was added and the suspension solution was stirred at ambient temperature for 1 hour. The suspension was filtered through Celite and washed with THE (100 mL). The filtrate was concentrated under reduced pressure to afford a crude amino alcohol intermediate as a colorless liquid which was dissolved in a mixture of dioxane (130 mL) and a saturated sodium bicarbonate aqueous solution (130 mL). Boc anhydride (5.6 g, 25.659 mmol) was added. The resulting solution was stirred at ambient temperature 16 hours. Water (100 mL) and ethyl acetate (200 mL) were added. The organic layer was separated, and aqueous layer was extracted with ethyl acetate (2×200 mL). The combined organic layers were dried over anhydrous sodium sulfate, filtered and concentrated under reduced pressure. The crude product was purified by flash chromatography (loaded in hexane) (330 g silica gel, eluting 0 to 30% EtOAc) to afford tert-butyl (6S,7R)-4-benzyl-6-hydroxy-7-isobutyl-1,4-diazepane-1-carboxylate (6.5889 g, 68%) as a white solid. ¹H NMR (250 MHz, DMSO-d₆) δ 7.48-7.05 (m, 5H), 5.00 (dd, J=16.2, 6.3 Hz, 1H), 3.99-3.35 (m, 5H), 2.91-2.53 (m, 3H), 2.23 (dt, J=12.3, 8.4 Hz, 2H), 1.61-1.17 (m, 12H), 1.00-0.71 (m, 6H). ESI-MS m/z calc. 362.25696, found 363.3 (M+1)⁺; Retention time: 1.86 minutes; LC method T.

### Step 3: (5R,6S)-1-Benzyl-5-isobutyl-1,4-diazepan-6-ol

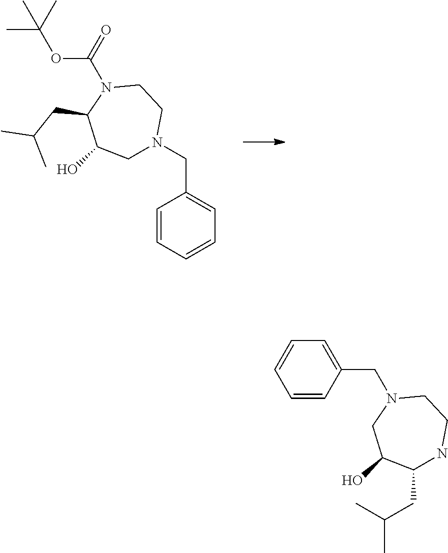 [SMILES: CC(C)C[C@@H]1[C@@H](O)CN(Cc2ccccc2)CCN1C(=O)OC(C)(C)C ; CC(C)C[C@H]1NCCN(Cc2ccccc2)C[C@@H]1O]

A 100 mL round bottom flask was charged with tert-butyl (6S,7R)-4-benzyl-6-hydroxy-7-isobutyl-1,4-diazepane-1-carboxylate (456 mg, 1.258 mmol), dioxane (4 mL) and HCl (4 mL of 4 M, 16.00 mmol) (4M dioxane solution). The mixture was stirred at rt for 3 hours. More HCl (4 mL of 4 M, 16.00 mmol) was added and the solution was stirred at rt for 3 hours. The volatiles were removed under reduced pressure. The solid was treated with DCM and hexanes and the solvents were evaporated. The operation was repeated 3 times. Drying in vacuo provided (5R,6S)-1-benzyl-5-isobutyl-1,4-diazepan-6-ol (Dihydrochloride salt) (425 mg, 100%) as a white solid. ¹H NMR (400 MHz, DMSO-d₆+10% D₂O) δ 7.64-7.44 (m, 5H), 4.46 (d, J=13.1 Hz, 1H), 4.32 (d, J=13.1 Hz, 1H), 4.07 (dd, J=6.8, 2.7 Hz, 1H), 3.68 (d, J=13.9 Hz, 1H), 3.62-3.35 (m, 5H), 3.29 (d, J=14.1 Hz, 1H), 1.63 (p, J=6.6 Hz, 1H), 1.44 (qt, J=14.2, 7.1 Hz, 2H), 0.90 (2 overlapped doublet, J=5.9 Hz, 6H). ESI-MS m/z calc. 262.2045, found 263.26 (M+1)⁺; Retention time: 0.5 minutes; LC method A.

### Step 4: 3-[(6S,7R)-4-Benzyl-6-hydroxy-7-isobutyl-1,4-diazepane-1-carbonyl]-N-[4-chloro-6-(2,6-dimethylphenyl)pyrimidin-2-yl]benzenesulfonamide

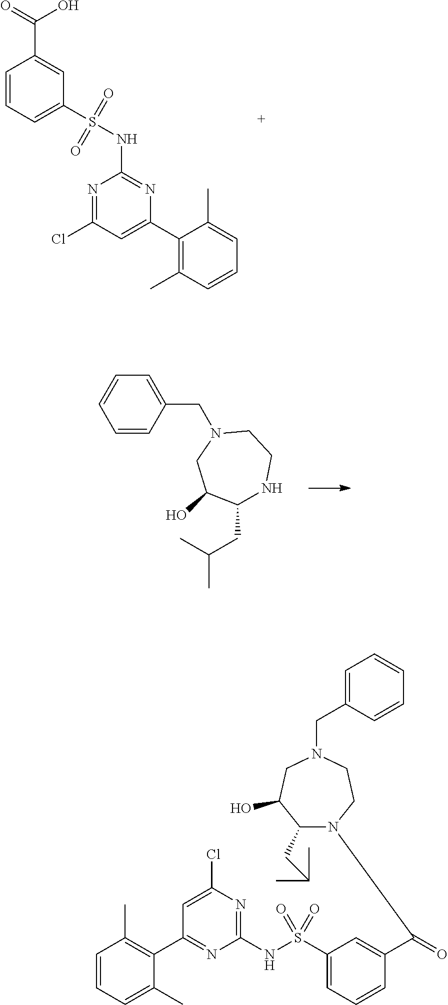 [SMILES: Cc1cccc(C)c1-c1cc(Cl)nc(NS(=O)(=O)c2cccc(C(=O)O)c2)n1 ; CC(C)C[C@H]1NCCN(Cc2ccccc2)C[C@@H]1O ; Cc1cccc(C)c1-c1cc(Cl)nc(NS(=O)(=O)c2cccc(C(=O)N3CCN(Cc4ccccc4)C[C@H](O)[C@H]3CC(C)C)c2)n1]

A 100 mL flask was charged under nitrogen with (5R,6R)-1-benzyl-5-isobutyl-1,4-diazepan-6-ol (Dihydrochloride salt) (425 mg, 1.255 mmol), anhydrous DMF (9 mL) and 3-[[4-chloro-6-(2,6-dimethylphenyl)pyrimidin-2-yl]sulfamoyl]benzoic acid (527 mg, 1.261 mmol). The suspension was cooled down in an ice bath. DIEA (1.5 mL, 8.612 mmol) (dissolved all solids in 1-2 min) followed by HATU (591 mg, 1.554 mmol) were added and the mixture was stirred at 0° C. for 20 min. The reaction mixture was poured in citric acid (50 mL of 10% w/v, 26.02 mmol)(10% aqueous) cooled in ice. The resulting white solid was filtered and washed with water. The wet solid was dissolved in DCM, dried over sodium sulfate and the solvent was evaporated. The resulting solid (903 mg) was purified by flash chromatography on silica gel (80 g column) using a gradient of methanol (0 to 5% over 30 min) in dichloromethane. The product eluted around 2% methanol to give 3-[(6S,7R)-4-benzyl-6-hydroxy-7-isobutyl-1,4-diazepane-1-carbonyl]-N-[4-chloro-6-(2,6-dimethylphenyl)pyrimidin-2-yl]benzenesulfonamide (546 mg, 65%) as a white solid. ESI-MS m/z calc. 661.24896, found 662.46 (M+1)⁺; Retention time: 1.47 minutes; LCMS Method A.

### Step 5: (16S,21R)-18-Benzyl-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 86)

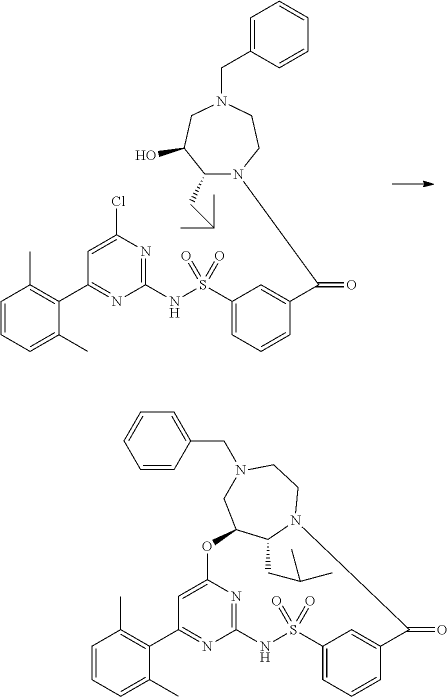 [SMILES: Cc1cccc(C)c1-c1cc(Cl)nc(NS(=O)(=O)c2cccc(C(=O)N3CCN(Cc4ccccc4)C[C@H](O)[C@H]3CC(C)C)c2)n1 ; Cc1cccc(C)c1-c1cc2nc(n1)NS(=O)(=O)c1cccc(c1)C(=O)N1CCN(Cc3ccccc3)C[C@H](O2)[C@H]1CC(C)C]

A 100 mL flask was charged under nitrogen with 3-[(6R,7R)-4-benzyl-6-hydroxy-7-isobutyl-1,4-diazepane-1-carbonyl]-N-[4-chloro-6-(2,6-dimethylphenyl)pyrimidin-2-yl]benzenesulfonamide (546 mg, 0.8245 mmol) and anhydrous DMF (50 mL). To the solution was added in one portion NaH (278 mg of 60% w/w, 6.951 mmol) (60% oil suspension) and the mixture was stirred under nitrogen at room temperature for 17 hours. A 10% aqueous solution of citric acid (200 mL of 10% w/v, 104.1 mmol) was cooled down in ice and the DMF reaction mixture was poured in the cold citric acid solution under stirring. The resulting solid was filtered (long filtration). The filtrate containing a 40:60 mixture of product and dimeric impurities was treated with 50 mL of brine and was extracted with EtOAc (3×50 mL). After drying over sodium sulfate, evaporation of the solvents gave a residue that was dissolved in DCM containing a bit of methanol, and it was purified by flash chromatography on silica gel (40 g column) using a gradient of methanol (0 to 10% over 60 min.) in dichloromethane. The product eluted around 3% methanol. Evaporation of the solvents gave (16S,21R)-18-benzyl-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (25 mg, 5%) as a white solid. ESI-MS m/z calc. 625.2723, found 626.53 (M+1)⁺; Retention time: 1.29 minutes; LC method A.

### Step 6: (16S,21R)-12-(2,6-Dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione

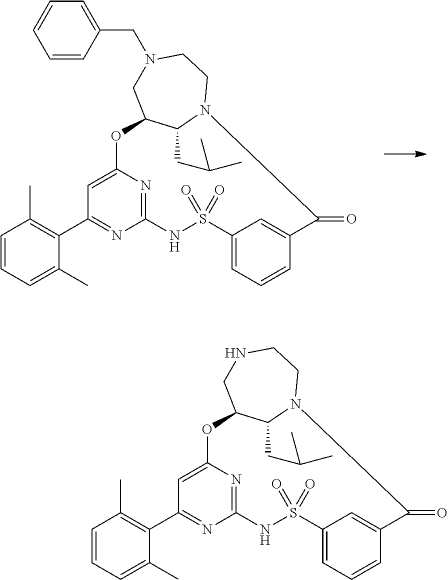 [SMILES: Cc1cccc(C)c1-c1cc2nc(n1)NS(=O)(=O)c1cccc(c1)C(=O)N1CCN(Cc3ccccc3)C[C@H](O2)[C@H]1CC(C)C ; Cc1cccc(C)c1-c1cc2nc(n1)NS(=O)(=O)c1cccc(c1)C(=O)N1CCNC[C@H](O2)[C@H]1CC(C)C]

A 100 mL flask was charged with (21R)-18-benzyl-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (21 mg, 0.03255 mmol) and MeOH (10 mL). The solution was sparged with nitrogen. Pd(OH)₂ (30 mg of 20% w/w, 0.04273 mmol) was added and the solution was stirred under an atmosphere of hydrogen (balloon) for 17 hours. The solution was purged with nitrogen. The catalyst was removed by filtration through Celite and the filtrate was concentrated. The residue was dissolved in DCM/MeOH and the solution was filtered through a syringe filter. Evaporation of the solvent gave (16S,21R)-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (18 mg, 103%) as an off-white solid. ESI-MS m/z calc. 535.22534, found 536.45 (M+1)⁺; Retention time: 1.07 minutes; LC method A.

### Step 7: (16S,21R)-18-(4,4-Difluorocyclohexyl)-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 85)

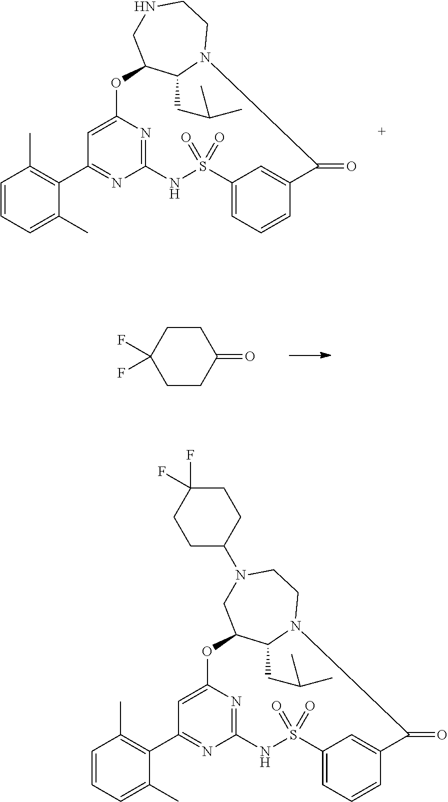 [SMILES: Cc1cccc(C)c1-c1cc2nc(n1)NS(=O)(=O)c1cccc(c1)C(=O)N1CCNC[C@H](O2)[C@H]1CC(C)C ; O=C1CCC(F)(F)CC1 ; Cc1cccc(C)c1-c1cc2nc(n1)NS(=O)(=O)c1cccc(c1)C(=O)N1CCN(C3CCC(F)(F)CC3)C[C@H](O2)[C@H]1CC(C)C]

A 4 mL vial was charged with (21R)-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (9 mg, 0.01680 mmol), anhydrous DCM (200 μL), 4,4-difluorocyclohexanone (18 mg, 0.1342 mmol) and acetic acid (10 μL, 0.1758 mmol). The vial was briefly purged with nitrogen, capped and stirred at room temperature for about 15 minutes. Sodium triacetoxyborohydride (10 mg, 0.04718 mmol) was added. The vial was purged with nitrogen, capped and the reaction was stirred at room temperature for 15 hours. A bit of methanol was added. The DCM was evaporated, and the residue was taken in DMSO (1 mL). The solution was microfiltered and purified by reverse phase preparative HPLC (Cis) using a gradient of acetonitrile in water (1 to 99% over 15 min) and HCl as a modifier to give (16S,21R)-18-(4,4-difluorocyclohexyl)-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (0.6 mg, 5%) as a white solid. ESI-MS m/z calc. 653.2847, found 654.51 (M+1)⁺; Retention time: 1.27 minutes; LC method A.

### Example 36: Preparation of Compound 87, Compound 84, Compound 88, and Compound 89

### Step 1: Ethyl 2-[benzyl-[(3R)-3-(tert-butoxycarbonylamino)-5-methyl-2-oxo-hexyl]amino]acetate

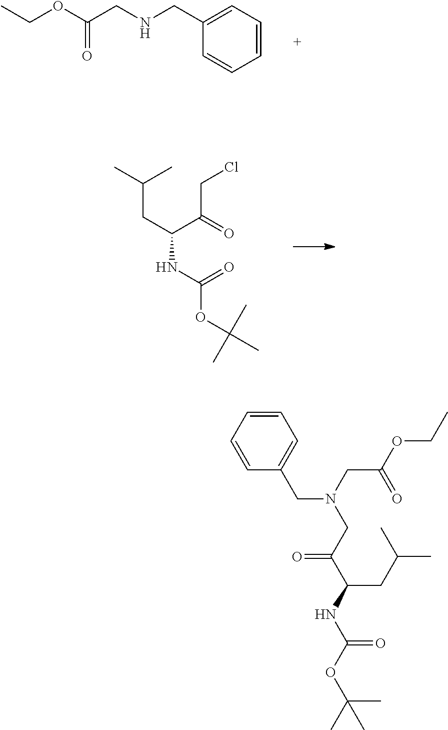 [SMILES: CCOC(=O)CNCc1ccccc1 ; CC(C)C[C@@H](NC(=O)OC(C)(C)C)C(=O)CCl ; CCOC(=O)CN(CC(=O)[C@@H](CC(C)C)NC(=O)OC(C)(C)C)Cc1ccccc1]

tert-Butyl N-[(1R)-1-(2-chloroacetyl)-3-methyl-butyl]carbamate (4 g, 15.165 mmol) was dissolved in DMF (25 mL) at RT. Ethyl 2-(benzylamino)acetate (hydrochloride salt) (2.93 g, 12.756 mmol) was added, followed by sodium bicarbonate (3.83 g, 45.592 mmol) and sodium iodide (1.13 g, 0.3082 mL, 7.5387 mmol). The reaction mixture was stirred at RT for 5 h. It was then partitioned between EtOAc (˜60 mL) and water (50 mL). The aqueous layer was extracted one more time with EtOAc. The combined organics were washed with water, brine and dried over sodium sulfate and was then filtrated and concentrated. The residue was purified using a silica gel column (0-15% EtOAc/Hexanes, compound visualized with iodine) to afford Ethyl 2-[benzyl-[(3R)-3-(tert-butoxycarbonylamino)-5-methyl-2-oxo-hexyl]amino]acetate (5 g, 74%) as a light yellowish oil. ESI-MS m/z calc. 420.26242, found 421.7 (M+1)⁺; Retention time: 3.02 minutes; LC method T.

### Step 2: 2-[Benzyl-[(3R)-3-(tert-butoxycarbonylamino)-5-methyl-2-oxo-hexyl]amino]acetic acid

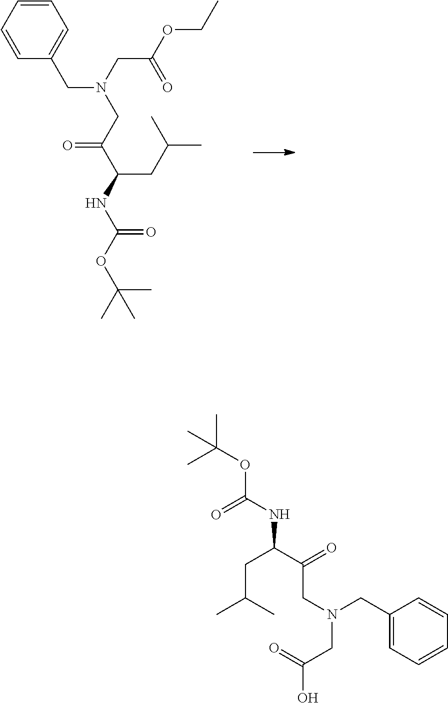 [SMILES: CCOC(=O)CN(CC(=O)[C@@H](CC(C)C)NC(=O)OC(C)(C)C)Cc1ccccc1 ; CC(C)C[C@@H](NC(=O)OC(C)(C)C)C(=O)CN(CC(=O)O)Cc1ccccc1]

Ethyl 2-[benzyl-[(3R)-3-(tert-butoxycarbonylamino)-5-methyl-2-oxo-hexyl]amino]acetate (5 g, 11.889 mmol) was dissolved in THE (50 mL) and MeOH (15 mL). The mixture was cooled in ice water bath. LiOH hydrate (711.79 mg, 29.722 mmol) in water (25 mL) was added by pipette. The cooling bath was removed. The mixture was stirred for 30 min. Water (100 mL) was added, followed by EtOAc (50 mL). 3 N HCl aqueous was added to adjust pH to 2. Layers were separated. The ethyl acetate layer was washed with brine, dried over anhydrous sodium sulfate, filtered and concentrated. The residue was treated with toluene (30 mL×2) and concentrated twice to give a yellow residue 2-[benzyl-[(3R)-3-(tert-butoxycarbonylamino)-5-methyl-2-oxo-hexyl]amino]acetic acid (5 g, 107%). ESI-MS m/z calc. 392.2311, found 393.6 (M+1)⁺; Retention time: 2.68 minutes; LC method T.

### Step 3: 2-[[(3R)-3-Amino-5-methyl-2-oxo-hexyl]-benzyl-amino]acetic acid

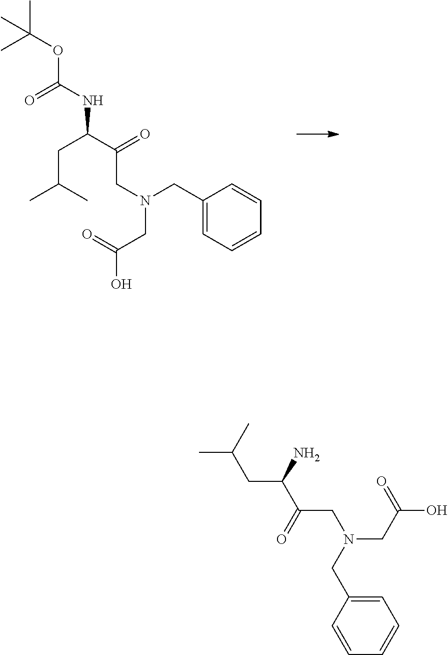 [SMILES: CC(C)C[C@@H](NC(=O)OC(C)(C)C)C(=O)CN(CC(=O)O)Cc1ccccc1 ; CC(C)C[C@@H](N)C(=O)CN(CC(=O)O)Cc1ccccc1]

2-[benzyl-[(3R)-3-(tert-butoxycarbonylamino)-5-methyl-2-oxo-hexyl]amino]acetic acid (5 g, 12.739 mmol)) was suspended in DCM (10 mL). HCl (20 mL of 4 M solution in dioxane, 80.000 mmol) was added. The mixture was stirred at RT for 2.5 h. It was then concentrated to remove most volatiles. The residue was placed under high vacuum for 2 hours to afford 2-[[(3R)-3-amino-5-methyl-2-oxo-hexyl]-benzyl-amino]acetic acid (4.5 g, 121%). ESI-MS m/z calc. 292.17868, found 293.4 (M+1)⁺; Retention time: 1.52 minutes; LC method T.

### Step 4: (7R)-4-Benzyl-7-isobutyl-1,4-diazepane-2,6-dione

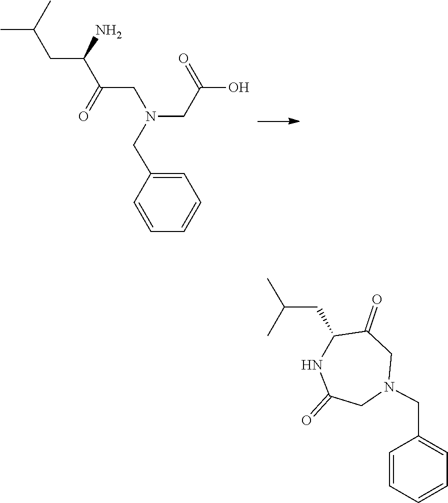 [SMILES: CC(C)C[C@@H](N)C(=O)CN(CC(=O)O)Cc1ccccc1 ; CC(C)C[C@H]1NC(=O)CN(Cc2ccccc2)CC1=O]

2-[[(3R)-3-amino-5-methyl-2-oxo-hexyl]-benzyl-amino]acetic acid (dihydrochloride salt) (4.5 g, 12.319 mmol) was dissolved in DMF (300 mL) at RT. HOAt (1.98 g, 14.547 mmol) was added, followed by HATU (5.62 g, 14.781 mmol). The mixture was stirred at RT for 1 h. DIEA (4.8230 g, 6.5 mL, 37.317 mmol) was added dropwise. The reaction mixture was stirred under nitrogen for 13 h. Water (200 mL) was added. The mixture was extracted with EtOAc (150 mL×3). The combined organics were concentrated. The residue was purified by silica gel chromatography, using 5-90% gradient to afford (7S)-4-benzyl-7-isobutyl-1,4-diazepane-2,6-dione (1.6 g, 42%) as a slightly yellow solid. ¹H NMR (250 MHz, Chloroform-d) 67.42-7.16 (m, 5H), 7.01 (s, 1H), 5.05-4.77 (m, 1H), 3.87-3.50 (m, 3H), 3.50-3.07 (m, 3H), 1.82-1.53 (m, 2H), 1.51-1.29 (m, 1H), 0.91 (d, J=6.2 Hz, 6H). ESI-MS m/z calc. 274.16812, found 275.4 (M+1)⁺; Retention time: 2.89 minutes; LC method T.

### Step 5: 1-Benzyl-5-(2-methylpropyl)-1,4-diazepan-6-ol

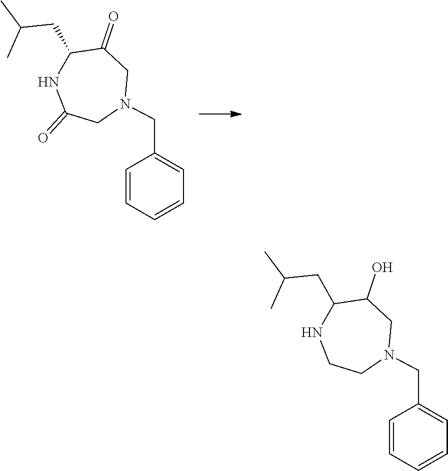 [SMILES: CC(C)C[C@H]1NC(=O)CN(Cc2ccccc2)CC1=O ; CC(C)CC1NCCN(Cc2ccccc2)CC1O]

(7R)-4-Benzyl-7-isobutyl-1,4-diazepane-2,6-dione (1.45 g, 5.2851 mmol) was dissolved in THE (35 mL) and the solution was cooled in ice water bath for 5 min. LAH (501.49 mg, 0.5469 mL, 13.213 mmol) was added in small portions over 10 min. The mixture was stirred at RT for 30 min, then placed in a 50° C. oil bath and stirred another 3 h. It was then cooled in ice. A saturated Rochelle's solution was added slowly (20 mL). The layers were separated, and the aqueous layer was extracted more (30 mL×2) using THF. The combined THE solution was concentrated and the residue was partitioned between chloroform (30 mL) and brine (30 mL). The layers were separated, and the aqueous layer extracted one more time using CHCl₃ (˜30 mL). The combined organics were concentrated, and the residue was purified by silica gel chromatography, using 0-5% MeOH in DCM. The product 1-benzyl-5-(2-methylpropyl)-1,4-diazepan-6-ol was isolated as a light-yellow resin (1.07 g, 78%). ¹H NMR (250 MHz, DMSO-d₆) δ 7.68-6.89 (m, 5H), 3.63 (s, 2H), 3.51-3.41 (m, 1H), 2.94-2.67 (m, 3H), 2.66-2.53 (m, 3H), 1.84-1.60 (m, 1H), 1.37-1.06 (m, 2H), 0.86 (dd, J=6.6, 4.7 Hz, 6H). ESI-MS m/z calc. 262.2045, found 264.3 (M+1)⁺; Retention time: 0.83 minutes; LC method W. Subsequent chemistry indicated a partial racemization of the isobutyl bearing chiral center.

### Step 6: 3-(4-Benzyl-6-hydroxy-7-isobutyl-1,4-diazepane-1-carbonyl)-N-[4-chloro-6-(2,6-dimethylphenyl)pyrimidin-2-yl]benzenesulfonamide

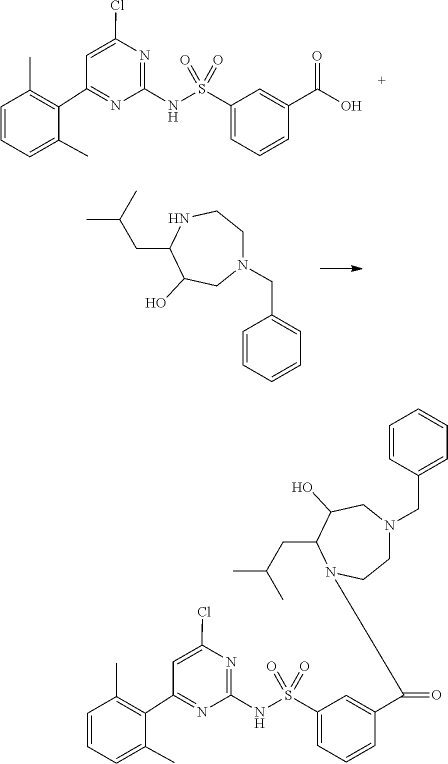 [SMILES: Cc1cccc(C)c1-c1cc(Cl)nc(NS(=O)(=O)c2cccc(C(=O)O)c2)n1 ; CC(C)CC1NCCN(Cc2ccccc2)CC1O ; Cc1cccc(C)c1-c1cc(Cl)nc(NS(=O)(=O)c2cccc(C(=O)N3CCN(Cc4ccccc4)CC(O)C3CC(C)C)c2)n1]

A 100 mL flask was charged under nitrogen with 1-benzyl-5-(2-methylpropyl)-1,4-diazepan-6-ol (566 mg, 2.157 mmol), 3-[[4-chloro-6-(2,6-dimethylphenyl)pyrimidin-2-yl]sulfamoyl]benzoic acid (760 mg, 1.819 mmol), potassium carbonate (840 mg, 6.078 mmol)(325 mesh) and anhydrous DCM (15 mL). DIC (0.34 mL, 2.171 mmol) was added and the heterogeneous mixture was vigorously stirred at room temperature for 16 hours. The reaction was diluted with DCM (40 mL) and it was quenched with a mixture of 10% aqueous citric acid solution and brine (40 mL). The two layers were separated. The aqueous layer was extracted with dichloromethane (2×30 mL-no product detected in aqueous phase at pH=4) and the combined organic layers were washed with brine (30 mL), dried over anhydrous sodium sulfate and concentrated. The residue was dissolved in MeOH and purified by reverse phase chromatography (100 g HP C₁₈ column) using a gradient of acetonitrile in water containing 5 mM HCl (10 to 100% over 25 min). The expected product eluted around 45-50% MeCN. The pure fractions were combined and concentrated. Brine was added and the product was extracted with DCM (3×30 mL). After drying over sodium sulfate, the solvents were evaporated to give 3-(4-benzyl-6-hydroxy-7-isobutyl-1,4-diazepane-1-carbonyl)-N-[4-chloro-6-(2,6-dimethylphenyl)pyrimidin-2-yl]benzenesulfonamide (358 mg, 30%) as a an amorphous yellow solid. ESI-MS m/z calc. 661.24896, found 662.5 (M+1)⁺; Retention time: 1.48 minutes; LC method A.

### Step 7: 18-Benzyl-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione, major diastereomer 1, cis configuration (Compound 88), and (16R,21R)-18-benzyl-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione (Compound 84), and (16S,21S)-18-benzyl-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 87), and 18-benzyl-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione, minor diastereomer 2 trans configuration (Compound 89)

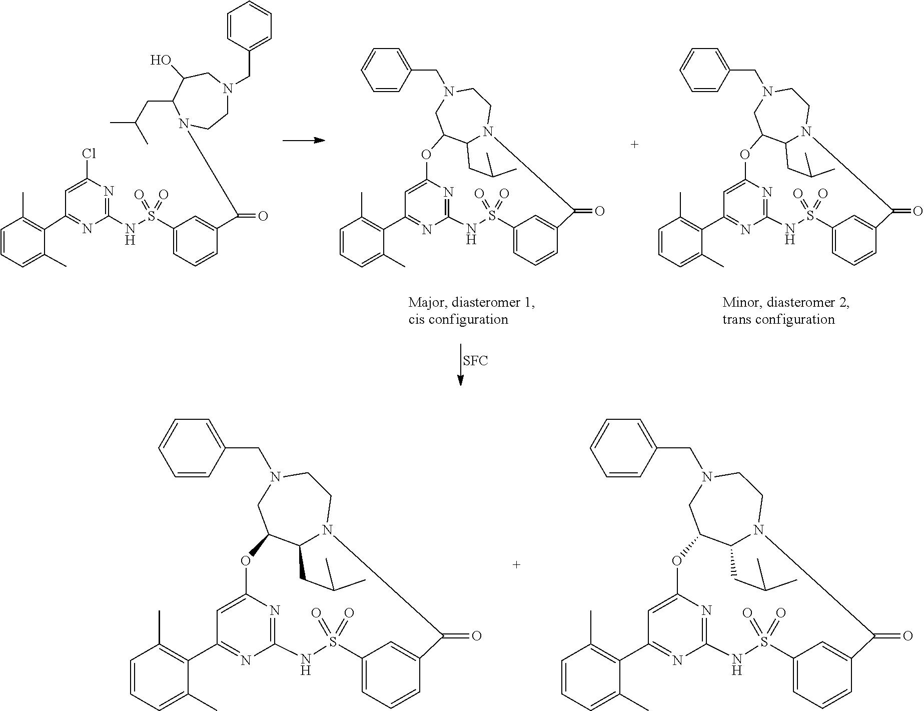 [SMILES: Cc1cccc(C)c1-c1cc(Cl)nc(NS(=O)(=O)c2cccc(C(=O)N3CCN(Cc4ccccc4)CC(O)C3CC(C)C)c2)n1 ; Cc1cccc(C)c1-c1cc2nc(n1)NS(=O)(=O)c1cccc(c1)C(=O)N1CCN(Cc3ccccc3)CC(O2)C1CC(C)C ; Cc1cccc(C)c1-c1cc2nc(n1)NS(=O)(=O)c1cccc(c1)C(=O)N1CCN(Cc3ccccc3)CC(O2)C1CC(C)C ; "SFC" ; Cc1cccc(C)c1-c1cc2nc(n1)NS(=O)(=O)c1cccc(c1)C(=O)N1CCN(Cc3ccccc3)C[C@H](O2)[C@@H]1CC(C)C ; Cc1cccc(C)c1-c1cc2nc(n1)NS(=O)(=O)c1cccc(c1)C(=O)N1CCN(Cc3ccccc3)C[C@@H](O2)[C@H]1CC(C)C]

A 100 mL flask was charged under nitrogen with 3-(4-benzyl-6-hydroxy-7-isobutyl-1,4-diazepane-1-carbonyl)-N-[4-chloro-6-(2,6-dimethylphenyl)pyrimidin-2-yl]benzenesulfonamide (358 mg, 0.5406 mmol) and anhydrous DMF (35 mL). To the solution was added NaH (173 mg of 60% w/w, 4.325 mmol) and the mixture was stirred under nitrogen at room temperature for 21 hours. A mixture of a 10% solution of citric acid (25 mL) and brine (75 mL) was cooled down in ice and the reaction mixture was transferred via pipette into this solution under stirring. The resulting white precipitate was filtered and dried (321 mg). The aqueous phase was neutralized to pH 7-8 by saturated sodium carbonate and the product was extracted with EtOAc (2×40 mL). After drying over sodium sulfate and evaporation of the solvents, 146 mg of solid was obtained. The filtered solid was dissolved in DCM and purified by flash chromatography on silica gel (24 g column) using a gradient of methanol (0 to 10% over 30 min) in dichloromethane. The product eluted around 3-4% MeOH to give 17 mg of relatively pure material. It was combined with the extracted fractions and purified a second time using a shallower gradient (0 to 10% over 60 min.). Two diastereomers were separated:

Major diastereomer (less polar), 18-benzyl-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (81 mg, 22%) was isolated as a white solid. ESI-MS m/z calc. 625.2723, found 626.6 (M+1)⁺; Retention time: 1.35 minutes (LC method A). This product was subjected to chiral SFC separation (Phenomenex LUX-1 (250×21.2 mm), μm column, mobile phase: 24% MeOH (20 mM NH₃), 76% CO₂, flow: 70 mL/min, 32 mg/mL in MeOH+20 mM NH₃:DMSO (90:10), injection volume 500 μL, pressure: 100 bar, wavelength: 210 nm) to give as peak 1: (16R,21R)-18-benzyl-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione (23 mg, 13%). ESI-MS m/z calc. 625.2723, found 626.4 (M+1)⁺; Retention time: 1.36 minutes (LC method A) and as peak 2, (16S,21S)-18-benzyl-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (23 mg, 13%). ESI-MS m/z calc. 625.2723, found 626.6 (M+1)⁺; Retention time: 1.35 minutes (LC method A).

Minor diastereomer (more polar) was further purified by preparative HPLC and flash chromatography on silica gel (4 g column) using a gradient of methanol (0 to 5% over 60 min) in dichloromethane. Evaporation of the solvents 18-benzyl-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (6 mg, 2%) as a white solid. ESI-MS m/z calc. 625.2723, found 626.5 (M+1)⁺; Retention time: 1.31 minutes, (LC method A).

### Example 37: Preparation of Compound 90, Compound 91, and Compound 92

### Step 1: 3-(4-Benzyl-6-hydroxy-7-isobutyl-1,4-diazepane-1-carbonyl)-N-[4-chloro-6-(2,6-dimethylphenyl)pyrimidin-2-yl]benzenesulfonamide

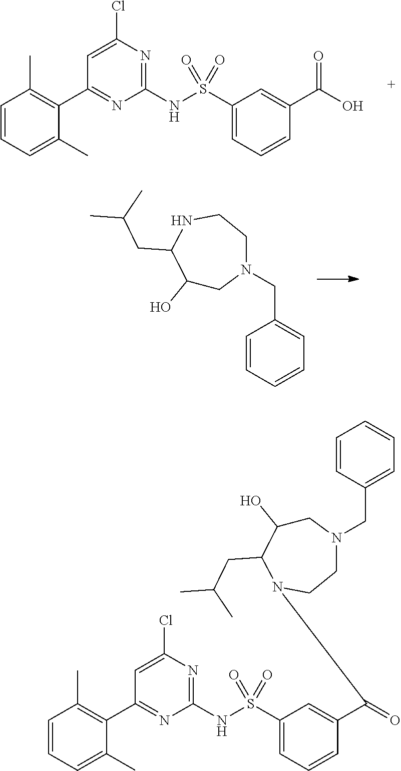 [SMILES: Cc1cccc(C)c1-c1cc(Cl)nc(NS(=O)(=O)c2cccc(C(=O)O)c2)n1 ; CC(C)CC1NCCN(Cc2ccccc2)CC1O ; Cc1cccc(C)c1-c1cc(Cl)nc(NS(=O)(=O)c2cccc(C(=O)N3CCN(Cc4ccccc4)CC(O)C3CC(C)C)c2)n1]

A 100 mL flask was charged under nitrogen with 1-benzyl-5-(2-methylpropyl)-1,4-diazepan-6-ol (420 mg, 1.601 mmol), 3-[[4-chloro-6-(2,6-dimethylphenyl)pyrimidin-2-yl]sulfamoyl]benzoic acid (560 mg, 1.340 mmol), potassium carbonate (589 mg, 4.262 mmol)(325 mesh) and anhydrous DCM (10 mL). DIC (0.25 mL, 1.597 mmol) was added and the heterogeneous mixture was vigorously stirred at room temperature for 24 h. The reaction was diluted with DCM (40 mL) and it was quenched with a mixture of 10% aqueous citric acid solution and brine (40 mL). The two layers were separated. The aqueous layer was extracted with dichloromethane (2×30 mL) and the combined organic layers were washed with brine (30 mL), dried over anhydrous sodium sulfate and concentrated. The residue was dissolved in MeOH and purified by reverse phase chromatography (100 g, C₁₈ column) using a gradient of acetonitrile in water (10 to 100% over 25 min), both solvents containing 0.1% of formic acid. The expected product eluted around 50-60% MeCN. The pure fractions were combined and concentrated. Brine was added and the product was extracted with DCM (3×30 mL). After drying over sodium sulfate, the solvents were evaporated to give 3-(4-benzyl-6-hydroxy-7-isobutyl-1,4-diazepane-1-carbonyl)-N-[4-chloro-6-(2,6-dimethylphenyl)pyrimidin-2-yl]benzenesulfonamide (350 mg, 39%) as a an amorphous tan solid. ESI-MS m/z calc. 661.24896, found 662.5 (M+1)⁺; Retention time: 1.49 minutes; LC method A.

### Step 2: 18-Benzyl-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione, mixture of diastereomer 1 and diastereomer 2 (87:13) (Compound 90)

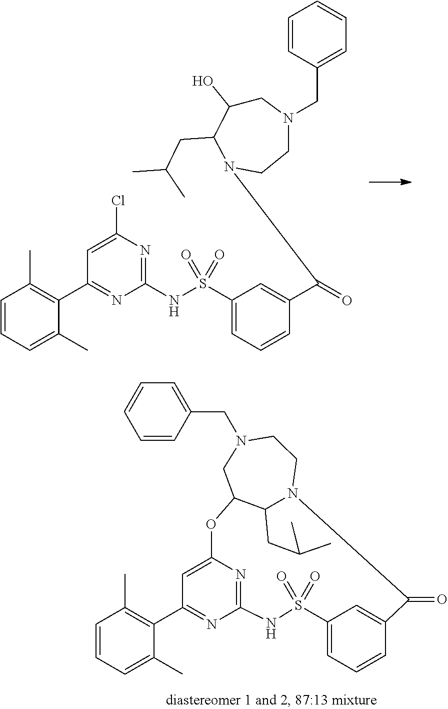 [SMILES: Cc1cccc(C)c1-c1cc(Cl)nc(NS(=O)(=O)c2cccc(C(=O)N3CCN(Cc4ccccc4)CC(O)C3CC(C)C)c2)n1 ; Cc1cccc(C)c1-c1cc2nc(n1)NS(=O)(=O)c1cccc(c1)C(=O)N1CCN(Cc3ccccc3)CC(O2)C1CC(C)C]

A 100 mL flask was charged under nitrogen with 3-(4-benzyl-6-hydroxy-7-isobutyl-1,4-diazepane-1-carbonyl)-N-[4-chloro-6-(2,6-dimethylphenyl)pyrimidin-2-yl]benzenesulfonamide (353 mg, 0.5330 mmol) and anhydrous DMF (35 mL). To the solution was added NaH (240 mg of 60% w/w, 6.001 mmol) and the mixture was stirred under nitrogen at room temperature for 24 hours. The mixture was cooled down to 0° C. and quenched by adding 10% citric acid. The mixture was diluted with water and brine (total 60 mL-final pH=5-6)). A white precipitate that formed was filtered and dried (442 mg). The product was dissolved in DCM and purified by flash chromatography on silica gel (24 g column) using a gradient of methanol (0 to 10% over 30 min) in dichloromethane. The product eluted around 3-4% MeOH to give 75 mg of relatively pure material (appear as 85:15 isomeric mixture). the product was dissolved in methanol and purified by reverse phase chromatography (15.5 g C₁₈ column) using a gradient of acetonitrile in water containing 5 mM of HCl (10 to 100% over 15 min). The product eluted around 45-55% MeCN. The pure fractions were collected, and the organic solvents removed by evaporation. Brine was added and the product was extracted with DCM (3×30 mL). After drying over sodium sulfate, the solvents were evaporated to give 60 mg (85:15 isomer mixture). The material was dissolved in MeOH (2 mL), microfiltered (0.45 uM) and purified by reverse phase preparative HPLC (Cis) using a gradient of acetonitrile in water (1 to 99% over 15 min) and HCl as a modifier. The pure fractions were collected, and the organic solvents removed by evaporation. Brine was added and the product was extracted with DCM (3×30 mL). After drying over sodium sulfate, the solvents were evaporated to give 18-benzyl-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (38 mg, 11%) as a white solid. >98% pure. ESI-MS m/z calc. 625.2723, found 626.6 (M+1)⁺; Retention time: 1.36 minutes. Apparent mixture of diastereomers (87:13). ¹H NMR (400 MHz, DMSO-d₆) complex spectra that suggest the presence of a major and minor isomer. δ 13.04 (s, 1H), 8.47 (s, 1H), 7.93 (s, 1H), 7.67 (s, 2H), 7.44-7.34 (m, 3H), 7.31-7.21 (m, 2H), 7.11 (d, J=7.6 Hz, 2H), 6.32 (s, 1H), 5.55 (s, 1H), 4.32-4.16 (m, 1H), 4.00-3.76 (m, 3H), 3.26-3.00 (m, 3H), 2.83-2.69 (m, 1H), 2.22-1.72 (m, 7H), 1.29-1.00 (m, 3H), 0.76-0.61 (m, 3H), 0.18 (d, J=6.2 Hz, 3H). ESI-MS m/z calc. 625.2723, found 626.6 (M+1)⁺; Retention time: 1.36 minutes; LC method A.

### Step 3: 12-(2,6-Dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione, mixture of diastereomer 1 and diastereomer 2 (87:13) (Compound 91)

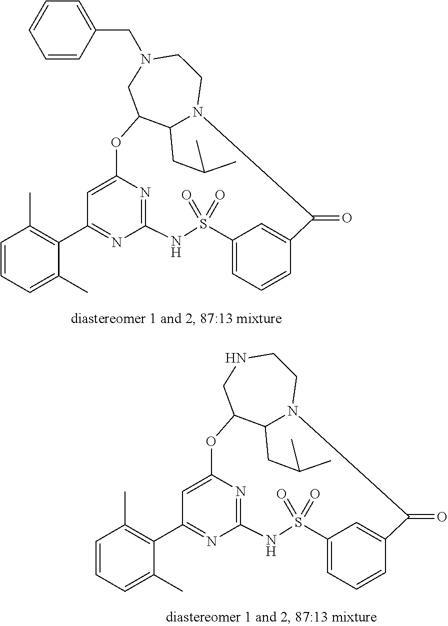 [SMILES: Cc1cccc(C)c1-c1cc2nc(n1)NS(=O)(=O)c1cccc(c1)C(=O)N1CCN(Cc3ccccc3)CC(O2)C1CC(C)C ; Cc1cccc(C)c1-c1cc2nc(n1)NS(=O)(=O)c1cccc(c1)C(=O)N1CCNCC(O2)C1CC(C)C]

A 100 mL flask was charged with 18-benzyl-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (31 mg, 0.04855 mmol) (diastereomer mixture 87:13) and MeOH (10 mL). The solution was sparged with nitrogen. Pd(OH)₂ (15 mg of 20% w/w, 0.02136 mmol) was added and the solution was stirred under an atmosphere of hydrogen (balloon) for 15 hours. The solution was purged with nitrogen. The catalyst was removed by filtration through Celite and the filtrate was concentrated to give 12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (26 mg, 98%) as a pure white solid. ESI-MS m/z calc. 535.22534, found 536.5 (M+1)⁺; Retention time: 1.11 minutes (LC method A). ¹H NMR (400 MHz, DMSO-d₆) small side peaks indicate presence of minor diastereomer. δ 8.65-8.37 (m, 1H), 7.86 (d, J=7.8 Hz, 1H), 7.68-7.50 (m, 2H), 7.20 (t, J=7.6 Hz, 1H), 7.08 (d, J=7.5 Hz, 2H), 6.13 (s, 1H), 5.56-5.32 (m, 1H), 4.76-3.95 (m, 3H), 3.27-3.07 (m, 4H), 2.94-2.81 (m, 1H), 1.98 (broad s, 6H), 1.73 (ddd, J=14.0, 10.2, 3.3 Hz, 1H), 1.21-1.08 (m, 1H), 1.02 (ddd, J=13.5, 9.8, 3.2 Hz, 1H), 0.68 (d, J=6.6 Hz, 3H), 0.27-0.11 (m, 3H).

### Step 4: 18-(3,3-Dimethylbutyl)-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione, major diastereomer 1 (Compound 92)

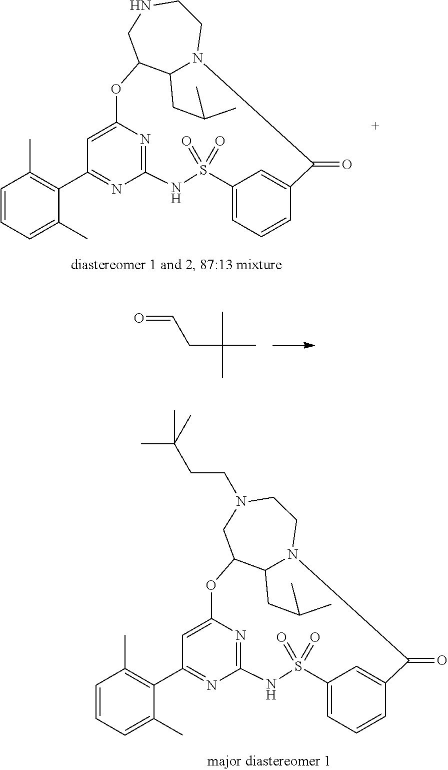

In a 4 mL vial, (21R)-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (22 mg, 0.04025 mmol) (87:13 mixture of diastereomers) was combined with 3,3-dimethylbutanal (22 μL, 0.1753 mmol) and acetic acid (15 μL, 0.2638 mmol) in dichloroethane (0.5 mL) and stirred under nitrogen (closed vial) at room temperature. After 2 hours, sodium cyanoborohydride (15 mg, 0.2387 mmol) was added and stirring at room temperature was continued for 1.5 hours The reaction mixture was then poured into aqueous ammonium chloride and extracted 3× with DCM The combined organics were dried over sodium sulfate, filtered and concentrated (27 mg). The resulting crude material was dissolved in DMSO, microfiltered and purified by reverse phase HPLC (1-99% ACN in water, HCl modifier, 15 min gradient). The main diastereomer gave 18-(3,3-dimethylbutyl)-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (4.1 mg, 15%) as a white solid. ESI-MS m/z calc. 619.3192, found 620.6 (M+1)⁺; Retention time: 1.38 minutes; LC method A.

### Example 38: Preparation of Compound 93 and Compound 94

### Step 1: Methyl (2S)-2-(tert-butoxycarbonylamino)-4-methyl-pentanoate

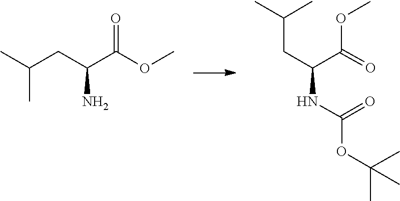

To a solution of methyl (2S)-2-amino-4-methyl-pentanoate (hydrochloride salt) (50 g, 269.74 mmol) in a of dioxane (313 mL) and aqueous sodium bicarbonate (1250 mL) at 0° C. was added Boc anhydride (62.4 g, 285.91 mmol). The resulting solution was stirred for 20 hours while being allowed to warm to ambient temperature. The reaction solution was extracted with ethyl acetate (3×500 mL). The combined organic layers were washed with brine (200 mL), dried over magnesium sulfate, filtered and concentrated under reduced pressure to afford methyl (2S)-2-(tert-butoxycarbonylamino)-4-methyl-pentanoate (50 g, 72%) as a pale-yellow oil. ¹H NMR (250 MHz, CDCl₃) δ 5.00-4.70 (m, 1H), 4.44-4.17 (m, 1H), 3.73 (s, 3H), 1.85-1.54 (m, 2H), 1.50-1.12 (m, 10H), 0.95 (d, J=2.3 Hz, 3H), 0.92 (d, J=2.3 Hz, 3H). ESI-MS m/z calc. 245.1627, found 246.1 (M+1)⁺; Retention time: 4.53 minutes; LC method S.

### Step 2: tert-Butyl N-[(1S)-1-(2-chloroacetyl)-3-methyl-butyl]carbamate

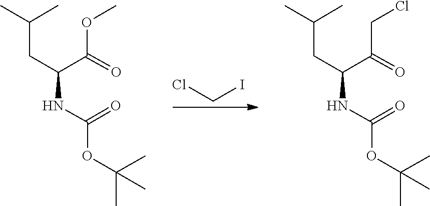

To a solution of diisopropyl amine (104.4 g, 144.60 mL, 1.0317 mol) in anhydrous THE (700 mL) at −50° C. was added n-BuLi (412.68 mL of 2.5 M, 1.0317 mol) slowly, then the reaction was warmed up to 0° C. and stirred for additional 30 minutes before it was cooled to −78° C. The LDA solution (kept at −78° C.) was added via canula to a precooled solution of methyl (2S)-2-(tert-butoxycarbonylamino)-4-methyl-pentanoate (50 g, 193.63 mmol) and chloro(iodo)methane (139.80 g, 57.721 mL, 776.74 mmol) in anhydrous THE (1300 mL) at −78° C., over 30 minutes. The resulting solution was stirred at this temperature for 1.5 hours. A mixture of acetic acid (164 mL) and THE (164 mL) was added slowly to quench the reaction. The reaction solution was warmed up to around 0° C., then water (1000 mL) was added and organic layer was separated. The aqueous layer was extracted with ethyl acetate (2×600 mL) Combined organic layer was washed with brine (400 mL), dried over sodium sulfate, filtered and concentrated under reduced pressure. The crude product (divided into two batches) was purified by flash chromatography (loaded in DCM) (330 g silica gel, eluting 0 to 15% EtOAc/hexanes) to tert-butyl N-[(1S)-1-(2-chloroacetyl)-3-methyl-butyl]carbamate (42.59 g, 79%) as a brown solid. ¹H NMR (250 MHz, CDCl₃) δ 5.06-4.75 (m, 1H), 4.53 (q, J=7.7, 5.9 Hz, 1H), 4.29 (d, J=2.9 Hz, 2H), 1.87-1.52 (m, 3H), 1.44 (s, 9H), 0.98 (d, J=4.7 Hz, 3H), 0.95 (d, J=4.7 Hz, 3H).

### Step 3: Ethyl 2-[benzyl-[(3S)-3-(tert-butoxycarbonylamino)-5-methyl-2-oxo-hexyl]amino]acetate

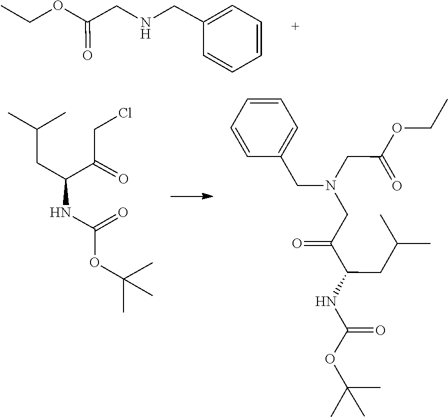

To a solution of ethyl 2-(benzylamino)acetate (28.476 g, 139.99 mmol) and tert-butyl N-[(1S)-1-(2-chloroacetyl)-3-methyl-butyl]carbamate (42.59 g, 153.40 mmol) in anhydrous DMF (280 mL) was added sodium bicarbonate (31.541 g, 375.46 mmol) followed by sodium iodide (13.801 g, 92.072 mmol). The resulting solution was stirred at ambient temperature for 48 hours before water (1000 mL) was added. The solution was extracted with ether (2×1000 mL). The combined organic layers were washed with brine (500 mL), dried over sodium sulfate, filtered and concentrated in vacuo to afford crude product. The crude product was purified (divided into two batches) by flash chromatography (loaded in DCM) (330 g silica gel, eluting 0 to 15% EtOAc/hexanes) to afford ethyl 2-[benzyl-[(3S)-3-(tert-butoxycarbonylamino)-5-methyl-2-oxo-hexyl]amino]acetate (54.69 g, 85%) as a pale-yellow liquid. ¹H NMR (250 MHz, CDCl₃) δ 7.57-7.05 (m, 5H), 4.97 (d, J=8.6 Hz, 1H), 4.40 (td, J=9.1, 4.0 Hz, 1H), 4.16 (q, J=7.1 Hz, 2H), 3.97-3.75 (m, 2H), 3.69 (s, 2H), 3.47 (d, J=2.3 Hz, 2H), 1.85-1.49 (m, 1H), 1.49-1.31 (m, 10H), 1.31-1.22 (m, 4H), 0.93 (d, J=6.6 Hz, 3H), 0.88 (d, J=6.6 Hz, 3H). ESI-MS m/z calc. 420.26242, found 421.3 (M+1)⁺; Retention time: 4.61 minutes; LC method S.

### Step 4: 2-[Benzyl-[(3S)-3-(tert-butoxycarbonylamino)-5-methyl-2-oxo-hexyl]amino]acetic acid

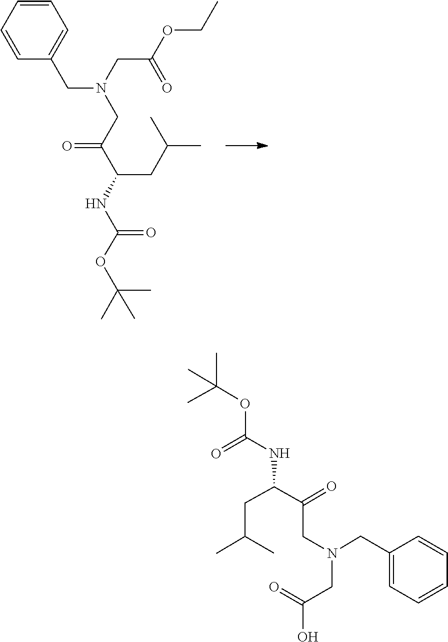 [SMILES: CCOC(=O)CN(CC(=O)[C@H](CC(C)C)NC(=O)OC(C)(C)C)Cc1ccccc1 ; CC(C)C[C@H](NC(=O)OC(C)(C)C)C(=O)CN(CC(=O)O)Cc1ccccc1]

Ethyl 2-[benzyl-[(3S)-3-(tert-butoxycarbonylamino)-5-methyl-2-oxo-hexyl]amino]acetate (5 g, 11.889 mmol) was dissolved in THE (50 mL), MeOH (15 mL) was added. The mixture was cooled in ice water bath. LiOH hydrate (1.4967 g, 35.667 mmol) mixture with water (25 mL) was added by pipette. The cooling bath was removed. The mixture was stirred another 30 min. Water (100 mL) was added, followed by EtOAc (50 mL). 3 N HCl aqueous was added to adjust the pH to 2. Layers were separated. The ethyl acetate layer was washed with brine, dried over anhydrous sodium sulfate, filtered and concentrated. The residue was treated with toluene (30 mL×2) and concentrated to give 2-[benzyl-[(3S)-3-(tert-butoxycarbonylamino)-5-methyl-2-oxo-hexyl]amino]acetic acid (5 g, 107%) as a pale yellow residue. ESI-MS m/z calc. 392.2311, found 393.6 (M+1)⁺; Retention time: 2.65 minutes.

### Step 5: 2-[[(3S)-3-Amino-5-methyl-2-oxo-hexyl]-benzyl-amino]acetic acid

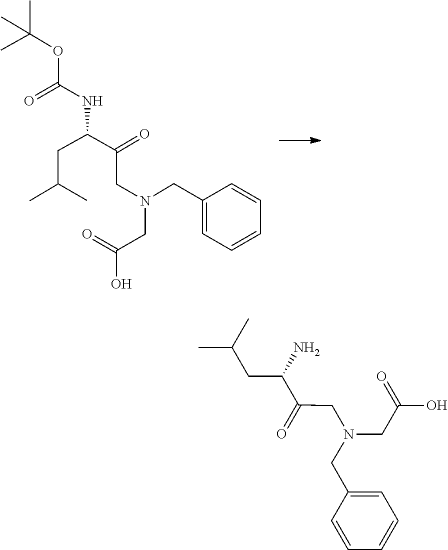 [SMILES: CC(C)C[C@H](NC(=O)OC(C)(C)C)C(=O)CN(CC(=O)O)Cc1ccccc1 ; CC(C)C[C@H](N)C(=O)CN(CC(=O)O)Cc1ccccc1]

2-[Benzyl-[(3S)-3-(tert-butoxycarbonylamino)-5-methyl-2-oxo-hexyl]amino]acetic acid (5 g, 12.739 mmol) was suspended in DCM (10 mL). HCl (20 mL of 4 M solution in dioxane, 80.000 mmol) was added. The mixture was stirred at RT for 2.5 h. It was then concentrated to remove most volatiles. The residue was placed under high vacuum for 2 hours to afford 2-[[(3S)-3-amino-5-methyl-2-oxo-hexyl]-benzyl-amino]acetic acid (dihydrochloride salt) (5.1 g, 99%) as pale-yellow solid. ESI-MS m/z calc. 292.17868, found 293.4 (M+1)⁺; Retention time: 1.52 minutes; LC method T.

### Step 6: (7S)-4-Benzyl-7-isobutyl-1,4-diazepane-2,6-dione

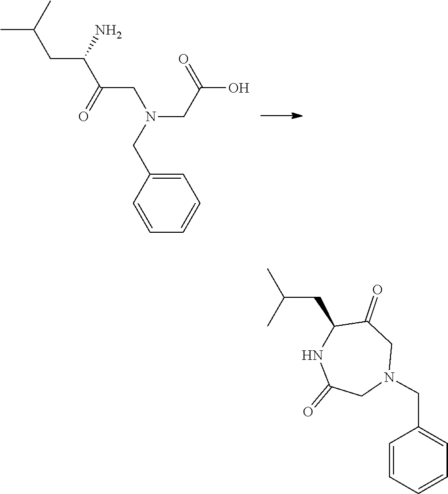 [SMILES: CC(C)C[C@H](N)C(=O)CN(CC(=O)O)Cc1ccccc1 ; CC(C)C[C@@H]1NC(=O)CN(Cc2ccccc2)CC1=O]

2-[[(3S)-3-amino-5-methyl-2-oxo-hexyl]-benzyl-amino]acetic acid (dihydrochloride salt) (5.1 g, 13.961 mmol) was dissolved in DMF (250 mL) at RT. HOAt (2.2803 g, 16.753 mmol) was added, followed by HATU (6.3700 g, 16.753 mmol). The mixture was stirred at RT for 1 h. DIEA (5.4131 g, 7.2953 mL, 41.883 mmol) was added dropwise. The reaction mixture was let to stir under nitrogen balloon for 13 h. Water (200 mL) was added. The mixture was extracted with EtOAc (150 mL×3). The combined organics were concentrated. The residue was purified by silica gel chromatography, using 5-90% gradient to afford (7S)-4-benzyl-7-isobutyl-1,4-diazepane-2,6-dione (1.6 g, 42%) as a slightly yellow solid. ESI-MS m/z calc. 274.16812, found 275.4 (M+1)⁺; Retention time: 2.89 minutes; LC method T.

### Step 7: 1-benzyl-5-(2-methylpropyl)-1,4-diazepan-6-ol

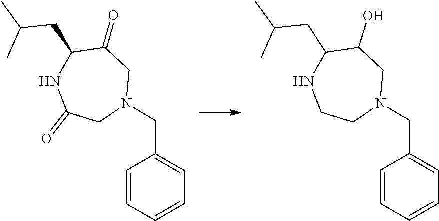 [SMILES: CC(C)C[C@@H]1NC(=O)CN(Cc2ccccc2)CC1=O ; CC(C)CC1NCCN(Cc2ccccc2)CC1O]

(7S)-4-Benzyl-7-isobutyl-1,4-diazepane-2,6-dione (1.2 g, 4.3738 mmol) was dissolved in THE (30 mL) and the solution was cooled in ice water bath for 5 min. LAH (415.03 mg, 0.4526 mL, 10.935 mmol) was added in small portions over 10 min. The mixture was stirred at RT for 30 min, then placed in a 50° C. oil bath and stirred for 3 h. It was then cooled in ice. A saturated Rochelle's salt solution was added slowly (20 mL). The layers were separated, and the aqueous layer was extracted more (30 mL×2) using THF. The combined THE solutions were concentrated and the residue was partitioned between chloroform (30 mL) and brine (30 mL). Layers were separated and the aqueous layer extracted one more time using CHCl₃ (˜30 mL). The combined organics were concentrated, and the residue was purified by silica gel chromatography, using 0-5% MeOH in DCM. The product 1-benzyl-5-(2-methylpropyl)-1,4-diazepan-6-ol was isolated as a light-yellow oil. ¹H NMR (250 MHz, DMSO-d₆) δ 7.67-6.86 (m, 5H), 3.64 (s, 2H), 3.47 (s, 1H), 2.93-2.80 (m, 1H), 2.74 (dd, J=12.9, 4.2 Hz, 2H), 2.68-2.53 (m, 3H), 1.71 (dp, J=13.3, 6.4 Hz, 1H), 1.43-1.04 (m, 2H), 0.98-0.68 (m, 6H). ESI-MS m/z calc. 262.2045, found 263.4 (M+1)⁺; Retention time: 0.87 minutes; LC method W. Subsequent work suggest a 70:30 diastereomeric mixture resulting from a partial racemization of the isobutyl bearing chiral center during a reaction.

### Step 8: 3-(4-Benzyl-6-hydroxy-7-isobutyl-1,4-diazepane-1-carbonyl)-N-[4-chloro-6-(2,6-dimethylphenyl)pyrimidin-2-yl]benzenesulfonamide

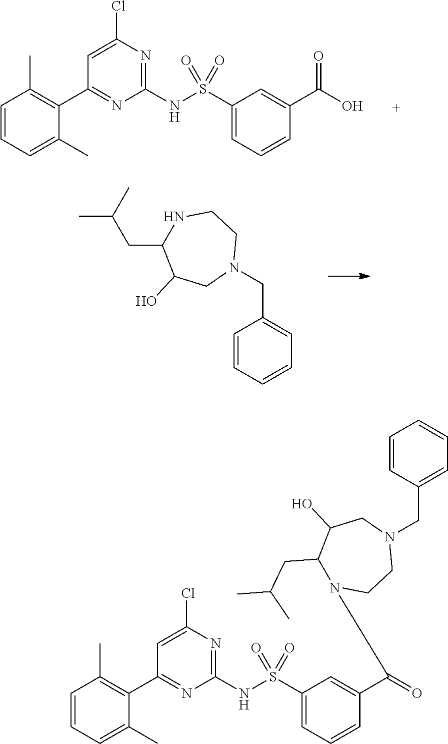 [SMILES: Cc1cccc(C)c1-c1cc(Cl)nc(NS(=O)(=O)c2cccc(C(=O)O)c2)n1 ; CC(C)CC1NCCN(Cc2ccccc2)CC1O ; Cc1cccc(C)c1-c1cc(Cl)nc(NS(=O)(=O)c2cccc(C(=O)N3CCN(Cc4ccccc4)CC(O)C3CC(C)C)c2)n1]

A 20 mL vial was charged under nitrogen with 1-benzyl-5-(2-methylpropyl)-1,4-diazepan-6-ol (261 mg, 0.9947 mmol), 3-[[4-chloro-6-(2,6-dimethylphenyl)pyrimidin-2-yl]sulfamoyl]benzoic acid (351 mg, 0.8400 mmol), potassium carbonate (342 mg, 2.475 mmol) and anhydrous DCM (6 mL). DIC (155 μL, 0.9899 mmol) was added and the heterogeneous mixture was vigorously stirred at room temperature for 17 hours. The reaction was diluted with DCM (40 mL) and it was quenched with a mixture of 10% aqueous citric acid solution and brine (40 mL). The two layers were separated. The aqueous layer was extracted with dichloromethane (3×25 mL) and the combined organic layers were washed with brine (30 mL), dried over anhydrous sodium sulfate and concentrated. The residue was dissolved in MeOH and purified by reverse phase chromatography (30 g C₁₈ column) using a gradient of acetonitrile in water (5 to 100% over 20 min). Mixed fractions were combined and purified a second time using the same eluant and 0.1% formic acid as a modifier. The expected product eluted around 50-60% MeCN. The pure fractions were combined and concentrated. Brine was added and the product was extracted with DCM (3×30 mL). After drying over sodium sulfate, the solvents were evaporated to give 3-(4-benzyl-6-hydroxy-7-isobutyl-1,4-diazepane-1-carbonyl)-N-[4-chloro-6-(2,6-dimethylphenyl)pyrimidin-2-yl]benzenesulfonamide (210 mg, 38%) as an amorphous solid. ¹HNMR of the product suggests a mixture of diastereomers in a 70:30 ratio. The material was used for next step without any further purification. ESI-MS m/z calc. 661.24896, found 662.5 (M+1)⁺; Retention time: 1.47 minutes; LC method A.

### Step 9: 18-Benzyl-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione, mixture of diastereomer 1 and diastereomer 2 (92:8) (Compound 94)

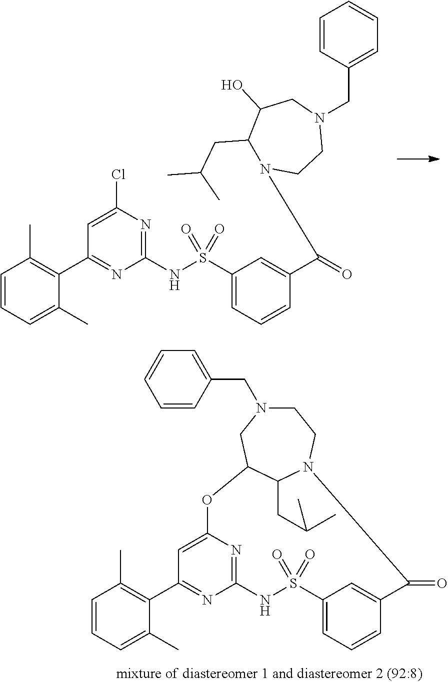 [SMILES: Cc1cccc(C)c1-c1cc(Cl)nc(NS(=O)(=O)c2cccc(C(=O)N3CCN(Cc4ccccc4)CC(O)C3CC(C)C)c2)n1 ; Cc1cccc(C)c1-c1cc2nc(n1)NS(=O)(=O)c1cccc(c1)C(=O)N1CCN(Cc3ccccc3)CC(O2)C1CC(C)C]

A 100 mL flask was charged under nitrogen with 3-(4-benzyl-6-hydroxy-7-isobutyl-1,4-diazepane-1-carbonyl)-N-[4-chloro-6-(2,6-dimethylphenyl)pyrimidin-2-yl]benzenesulfonamide (207 mg, 0.3126 mmol) and anhydrous DMF (20 mL). To the solution was added NaH (143 mg of 60% w/w, 3.575 mmol) and the mixture was stirred under nitrogen at room temperature for 23 hours. The mixture was cooled down to 0° C. and quenched by adding 10% citric acid. The mixture was diluted with water and brine (total 60 mL). A white precipitate that formed was filtered and dried (241 mg crude). The product was dissolved in DCM and purified by flash chromatography on silica gel (24 g column) using a gradient of methanol (0 to 10% over 30 min) in dichloromethane. The product eluted around 3-4% MeOH to give 52 mg of relatively pure material (appear as 93:7 isomeric mixture). the product was dissolved in methanol and purified by reverse phase chromatography (15.5 g C₁₈ column) using a gradient of acetonitrile in water containing 5 mM of HCl (10 to 100% over 15 min). The product eluted around 50-55% MeCN. The pure fractions were collected, and the organic solvents removed by evaporation. Brine was added and the product was extracted with DCM (3×30 mL). After drying over sodium sulfate, the solvents were evaporated to give 18-benzyl-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (42 mg, 21%) as a white solid. Mixture of 2 diastereomers (92:8). ¹H NMR (400 MHz, DMSO-d₆) complex spectra that suggest the presence of a major and minor isomer. δ 13.03 (broad s, 1H), 8.48 (s, 1H), 7.93 (s, 1H), 7.68 (s, 2H), 7.45-7.34 (m, 3H), 7.26 (q, J=7.8 Hz, 2H), 7.11 (d, J=7.5 Hz, 2H), 6.33 (s, 1H), 5.55 (s, 1H), 4.34-4.10 (m, 1H), 4.01-3.69 (m, 3H), 3.27-2.95 (m, 4H), 2.87-2.70 (m, 1H), 2.28-1.70 (m, 7H), 1.35-1.00 (m, 3H), 0.73-0.60 (m, 3H), 0.18 (d, J=6.2 Hz, 3H). ESI-MS m/z calc. 625.2723, found 626.6 (M+1)⁺; Retention time: 1.37 minutes; LC method A.

### Step 10: 12-(2,6-Dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 93)

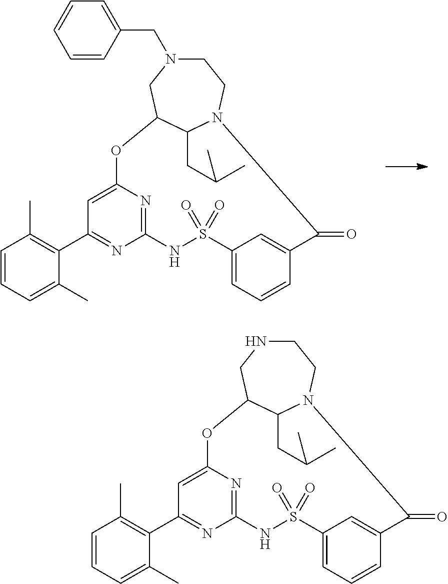

A 100 mL flask was charged with 18-benzyl-12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (12 mg, 0.01812 mmol) (diastereomeric mixture 80:20) and MeOH (5 mL). The solution was sparged with nitrogen. Pd(OH)₂ (14 mg of 20% w/w, 0.01994 mmol) was added and the solution was stirred under an atmosphere of hydrogen (balloon) for 23 hours. The solution was purged with nitrogen. The catalyst was removed by filtration through Celite and the filtrate was concentrated to give 9 mg of residue. The product was dissolved in DMSO (1 mL), micro filtered and purified by reverse phase preparative HPLC (Cis) using a gradient of acetonitrile in water (1 to 99% over 15 min) and HCl as a modifier. Evaporation of the solvents by genevac evaporation gave 12-(2,6-dimethylphenyl)-21-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (4.8 mg, 44%) as a white solid. ESI-MS m/z calc. 535.22534, found 536.6 (M+1)⁺; Retention time: 1.1 minutes; LC method A.

### Example 39: Preparation of Compound 95 and Compound 96

### Step 1: Methyl (2S)-2-(benzylamino)-4-methyl-pentanoate

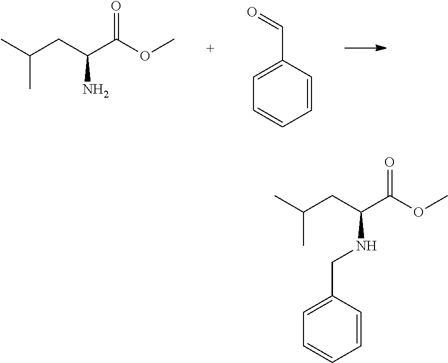

A solution of methyl (2S)-2-amino-4-methyl-pentanoate (18.537, 0.1 mol), triethylamine (10.119 g, 13.938 mL, 100.00 mmol) and anhydrous magnesium sulfate (20 g, 166.16 mmol) in methanol (200 mL) was stirred for approximately 10 minutes at room temperature. Benzaldehyde (10.612 g, 10.204 mL, 100.00 mmol) was added and the reaction mixture was stirred at room temperature for 16 hours. The reaction mixture was then filtered through a pad of Celite to remove the magnesium sulfate, then it was cooled in an ice bath. Sodium borohydride (7.5665 g, 200.00 mmol) was added to the reaction mixture slowly. Gas generated during the addition. The reaction mixture was stirred at room temperature for 1 hour. The reaction mixture was quenched with saturated ammonium chloride (100 mL) to adjust pH to 7. The volatiles were removed under vacuum. The aqueous residue was extracted with diethyl ether (3×200 mL). The combined ether layers were washed with water (200 mL) and brine (200 mL), dried over anhydrous magnesium sulfate and concentrated under vacuum. The residue was purified by silica gel chromatography using 0 to 20% hexane-ethyl acetate to furnish methyl (2S)-2-(benzylamino)-4-methyl-pentanoate (10.851 g, 46%) as a clear liquid. ESI-MS m/z calc. 235.15723, found 236.2 (M+1)⁺; Retention time: 2.87 minutes; LC method S.

### Step 2: (3S,6S)-4-Benzyl-6-hydroxy-3-isobutyl-1,4-diazepan-2-one

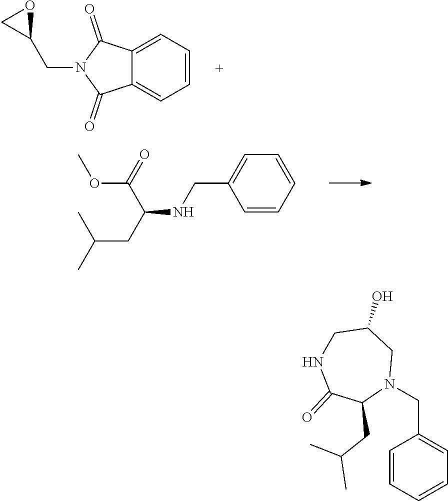

To a solution of methyl (2S)-2-(benzylamino)-4-methyl-pentanoate (18.83 g, 80.018 mmol) and 2-[[(2S)-oxiran-2-yl]methyl]isoindoline-1,3-dione (16.259 g, 80.018 mmol) in ACN (112.98 mL) was added magnesium perchlorate (26.791 g, 120.03 mmol). The reaction was stirred at room temperature for 24 hours. The reaction was diluted with water (500 mL) and extracted with dichloromethane (3×500 mL). The combined organic layers were washed with brine (500 mL), dried over anhydrous sodium sulfate and concentrated under vacuum. The residue was diluted with methanol (377 mL). hydrazine hydrate (10.975 g, 160.04 mmol) was added to the reaction mixture. The reaction was stirred at 65° C. for 2 days. The white precipitate was filtered off, and the filtrate was concentrated under vacuum. The residue was diluted with 1N NaOH (aqueous) (200 mL), and it was extracted with ethyl acetate (3×200 mL). The combined organic layers were washed with brine (200 mL), dried over anhydrous sodium sulfate and concentrated under vacuum. The residue was purified by silica gel chromatography using 50 to 100% hexane-ethyl acetate to furnish (3S,6S)-4-benzyl-6-hydroxy-3-isobutyl-1,4-diazepan-2-one (10.34 g, 44%) as an off-white solid. ¹H NMR (250 MHz, CDCl₃) δ 7.38-7.27 (m, 5H), 6.02 (t, J=6.8 Hz, 1H), 3.90-3.78 (m, 2H), 3.72 (dd, J=8.6, 6.1 Hz, 1H), 3.52 (d, J=13.9 Hz, 1H), 3.36 (t, J=6.6 Hz, 2H), 3.19 (dd, J=14.3, 3.7 Hz, 1H), 2.67 (dd, J=14.3, 9.5 Hz, 1H), 1.94-1.80 (m, 1H), 1.74-1.66 (m, 1H), 1.62-1.46 (m, 1H), 0.95 (dd, J=6.6, 5.6 Hz, 6H). ESI-MS m/z calc. 276.18378, found 277.0 (M+1)⁺; Retention time: 2.25 minutes; LC method S.

### Step 3: (3S)-4-Benzyl-6-hydroxy-3-isobutyl-1,4-diazepan-2-one

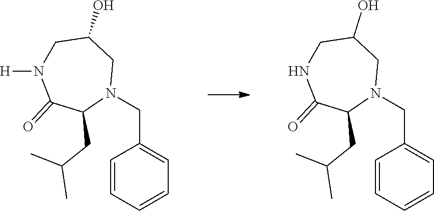 [SMILES: CC(C)C[C@H]1C(=O)NC[C@H](O)CN1Cc1ccccc1 ; CC(C)C[C@H]1C(=O)NCC(O)CN1Cc1ccccc1]

Into a solution of (3S,6S)-4-benzyl-6-hydroxy-3-isobutyl-1,4-diazepan-2-one (5.63 g, 20.371 mmol) in DCM (112 mL) was added Dess-Martin periodinane (12.960 g, 30.556 mmol) at 0° C. The reaction was stirred at room temperature for 2 hours. The reaction was quenched with a mixture of saturated 1:1 mixture of Na₂S₂O₃ and sodium bicarbonate (100 mL), and it was extracted with DCM (3×100 mL). The combined organic layers were washed with brine (100 mL), dried over anhydrous sodium sulfate and concentrated in vacuo. The residue was dissolved in methanol (281 mL). Sodium borohydride (2.3121 g, 2.4467 mL, 61.113 mmol) was added to the reaction mixture at room temperature and stirred for 1 hour. The reaction was quenched with water (100 mL). The product was extracted with ethyl acetate (3×200 mL). The combined organic layers were washed with brine (100 mL), dried over anhydrous sodium sulfate and concentrated under vacuum. The residue was purified by silica gel chromatography using 50 to 100% hexane-ethyl acetate to furnish (3S)-4-benzyl-6-hydroxy-3-isobutyl-1,4-diazepan-2-one (1.793 g, 32%). ¹H NMR (250 MHz, CDCl₃) δ 7.52-6.96 (m, 5H), 6.12 (s, 1H), 3.99-2.51 (m, 8H), 1.94-1.63 (m, 2H), 1.63-1.37 (m, 1H), 1.05-0.68 (m, 6H). ESI-MS m/z calc. 276.18378, found 276.9 (M+1)⁺; Retention time: 2.22 minutes; LC method S. Mixture of diastereomers.

### Step 4: (2S)-1-Benzyl-2-isobutyl-1,4-diazepan-6-ol

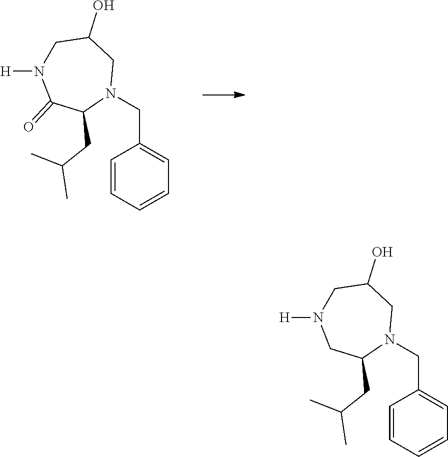 [SMILES: CC(C)C[C@H]1C(=O)NCC(O)CN1Cc1ccccc1 ; CC(C)C[C@H]1CNCC(O)CN1Cc1ccccc1]

Into a solution of (3S)-4-benzyl-6-hydroxy-3-isobutyl-1,4-diazepan-2-one (1.793 g, 6.4227 mmol) in anhydrous THF (40 mL) was added LAH (1.4626 g, 1.5950 mL, 38.536 mmol). The reaction mixture was stirred at 40° C. for 2 days. The reaction was quenched with water (1.5 mL), 15% NaOH (aqueous) (1.5 mL) and water (4.5 mL) subsequently. After stirring at room temperature for 1 hour, the white precipitate was removed by filtration through a pad of Celite. The filtrate was dried over anhydrous magnesium sulfate and concentrated in vacuo to furnish (2S)-1-benzyl-2-isobutyl-1,4-diazepan-6-ol (1.598 g, 95%) as a mixture of diastereomers. ESI-MS m/z calc. 262.2045, found 263.2 (M+1)⁺; Retention time: 1.96 minutes; LC method S.

### Step 5: tert-Butyl (3S)-4-benzyl-6-hydroxy-3-isobutyl-1,4-diazepane-1-carboxylate

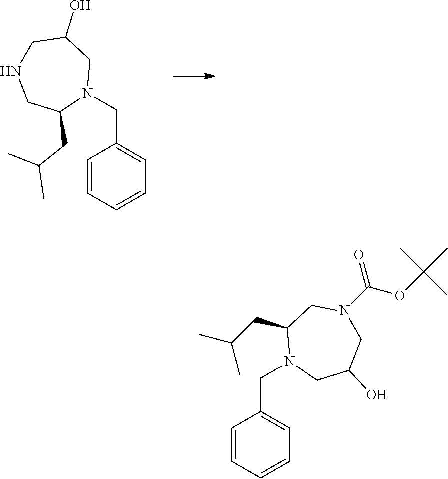 [SMILES: CC(C)C[C@H]1CNCC(O)CN1Cc1ccccc1 ; CC(C)C[C@H]1CN(C(=O)OC(C)(C)C)CC(O)CN1Cc1ccccc1]

Into a solution of (2S)-1-benzyl-2-isobutyl-1,4-diazepan-6-ol (1.598 g, 6.0902 mmol) in dichloromethane (20 mL), was added Boc anhydride (1.9938 g, 9.1353 mmol) and triethylamine (0.924 g, 9.1353 mmol). The reaction was stirred at room temperature for 2 hours. The reaction was quenched with brine (50 mL). The two layers were separated, and the aqueous layer was extracted with dichloromethane (2×50 mL). The combined organic layers were dried over anhydrous magnesium sulfate and concentrated under vacuum. The residue was purified by silica gel chromatography using 0 to 100% hexane-diethyl ether to furnish tert-butyl (3S)-4-benzyl-6-hydroxy-3-isobutyl-1,4-diazepane-1-carboxylate (1.359 g, 61%) as a clear gel and as a mixture of diastereomers. ESI-MS m/z calc. 362.25696, found 363.3 (M+1)⁺; Retention time: 3.88 minutes; LC method S.

### Step 6: tert-Butyl (3S)-6-hydroxy-3-isobutyl-1,4-diazepane-1-carboxylate

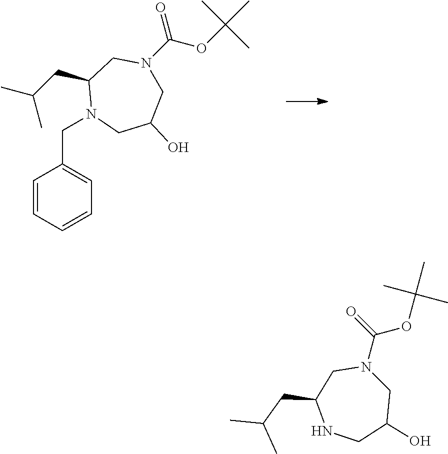 [SMILES: CC(C)C[C@H]1CN(C(=O)OC(C)(C)C)CC(O)CN1Cc1ccccc1 ; CC(C)C[C@H]1CN(C(=O)OC(C)(C)C)CC(O)CN1]

Into a solution of tert-butyl (3S)-4-benzyl-6-hydroxy-3-isobutyl-1,4-diazepane-1-carboxylate (647 mg, 1.7848 mmol) in methanol (50 mL) was added 10% Palladium on carbon (190 mg) and ammonium formate (337.63 mg, 5.3544 mmol). The reaction mixture was stirred at 65° C. for 2 hours. After cooled down to room temperature, the catalyst was removed by filtration through a pad of Celite. The filtrate was concentrated under vacuum. The residue was diluted with dichloromethane (50 mL) and washed with water (20 mL) and brine (20 mL), dried over anhydrous sodium sulfate and concentrated under vacuum. The residue was purified by silica gel chromatography using 0 to 20% dichloromethane in methanol (buffered with 0.5% ammonium hydroxide) to furnish tert-butyl (3S)-6-hydroxy-3-isobutyl-1,4-diazepane-1-carboxylate (382.4 mg, 78%) as a clear oil and as a mixture of diastereomers. ¹H NMR (250 MHz, DMSO-d₆) δ 4.64 (m, 1H), 3.96-3.43 (m, 3H), 3.05-2.61 (m, 3H), 2.35 (m, 1H), 1.98 (s, 1H), 1.79-1.54 (m, 1H), 1.38 (s, 9H), 1.24-0.96 (m, 2H), 0.85 (t, J=6.8 Hz, 6H). ESI-MS m/z calc. 272.21, found 273.2 (M+1)⁺; Retention time: 2.95 minutes; LC method S.

### Step 7: tert-Butyl (3S,6R)-4-[3-[[4-chloro-6-(2,6-dimethylphenyl)pyrimidin-2-yl]sulfamoyl]benzoyl]-6-hydroxy-3-isobutyl-1,4-diazepane-1-carboxylate and tert-butyl (3S,6S)-4-[3-[[4-chloro-6-(2,6-dimethylphenyl)pyrimidin-2-yl]sulfamoyl]benzoyl]-6-hydroxy-3-isobutyl-1,4-diazepane-1-carboxylate

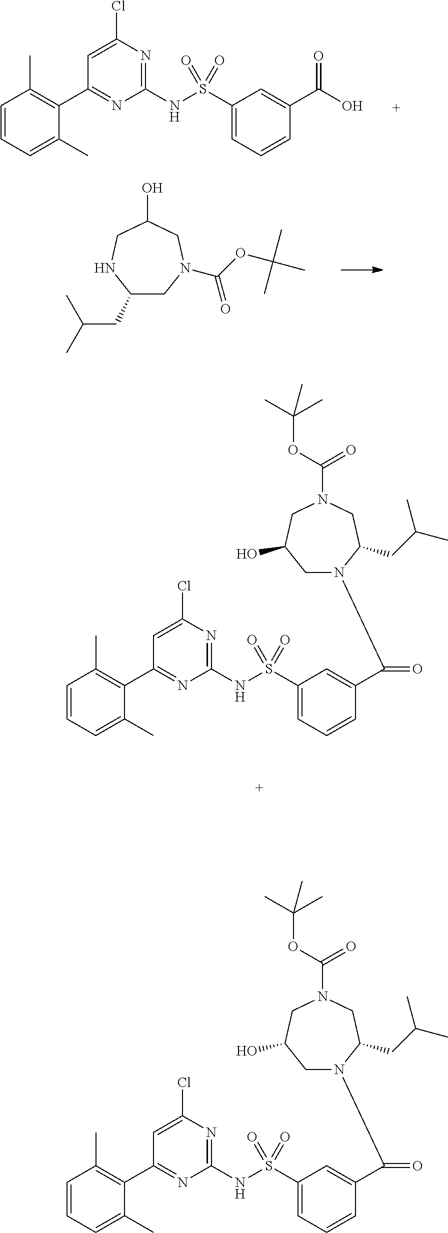 [SMILES: Cc1cccc(C)c1-c1cc(Cl)nc(NS(=O)(=O)c2cccc(C(=O)O)c2)n1 ; CC(C)C[C@H]1CN(C(=O)OC(C)(C)C)CC(O)CN1 ; Cc1cccc(C)c1-c1cc(Cl)nc(NS(=O)(=O)c2cccc(C(=O)N3C[C@@H](O)CN(C(=O)OC(C)(C)C)C[C@@H]3CC(C)C)c2)n1 ; Cc1cccc(C)c1-c1cc(Cl)nc(NS(=O)(=O)c2cccc(C(=O)N3C[C@H](O)CN(C(=O)OC(C)(C)C)C[C@@H]3CC(C)C)c2)n1]

A 100 mL flask was charged under nitrogen with tert-butyl (3S)-6-hydroxy-3-isobutyl-1,4-diazepane-1-carboxylate (371 mg, 1.362 mmol) (1:1 mixture of diastereomers), anhydrous DMF (8 mL) and 3-[[4-chloro-6-(2,6-dimethylphenyl)pyrimidin-2-yl]sulfamoyl]benzoic acid (474 mg, 1.134 mmol). After dissolution of the reagents, the mixture was cooled down in an ice bath. DIEA (1.3 mL, 7.463 mmol) and HATU (525 mg, 1.381 mmol) were added and the mixture was stirred at 0° C. for 4 hours. 2 isomers (1:1 ratio) were detected by LCMS. The reaction was quenched by being poured in citric acid (50 mL of 10% w/v, 26.02 mmol)(10% aqueous) under vigorous stirring and cooled in ice. The resulting white solid was filtered and dried. The solid was dissolved in DCM and it was purified by flash chromatography on silica gel (80 g column) using a gradient of methanol (0 to 5% over 30 min) in dichloromethane. Two isomers were isolated: less polar diastereomer, first to elute, tert-butyl (3S,6R)-4-[3-[[4-chloro-6-(2,6-dimethylphenyl)pyrimidin-2-yl]sulfamoyl]benzoyl]-6-hydroxy-3-isobutyl-1,4-diazepane-1-carboxylate (240 mg, 63%). ESI-MS m/z calc. 671.25446, found 672.36 (M+1)⁺; Retention time: 1.98 minutes (LC method A); and a more polar, second to elute diastereomer tert-butyl (3S,6S)-4-[3-[[4-chloro-6-(2,6-dimethylphenyl)pyrimidin-2-yl]sulfamoyl]benzoyl]-6-hydroxy-3-isobutyl-1,4-diazepane-1-carboxylate (294 mg, 77%). ESI-MS m/z calc. 671.25446, found 672.36 (M+1)⁺; Retention time: 1.94 minutes (LC method A).

### Step 8: tert-Butyl (16S,20S)-12-(2,6-dimethylphenyl)-20-(2-methylpropyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (Compound 96)

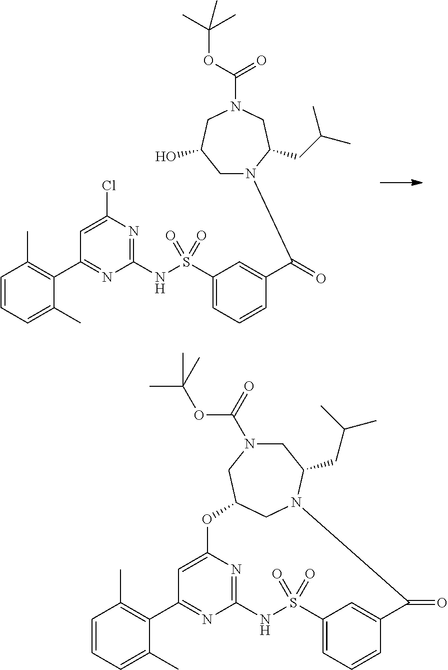 [SMILES: Cc1cccc(C)c1-c1cc(Cl)nc(NS(=O)(=O)c2cccc(C(=O)N3C[C@H](O)CN(C(=O)OC(C)(C)C)C[C@@H]3CC(C)C)c2)n1 ; Cc1cccc(C)c1-c1cc2nc(n1)NS(=O)(=O)c1cccc(c1)C(=O)N1C[C@@H](CN(C(=O)OC(C)(C)C)C[C@@H]1CC(C)C)O2]

A 100 mL flask was charged under nitrogen with tert-butyl (3S,6S)-4-[3-[[4-chloro-6-(2,6-dimethylphenyl)pyrimidin-2-yl]sulfamoyl]benzoyl]-6-hydroxy-3-isobutyl-1,4-diazepane-1-carboxylate (294 mg, 0.4373 mmol) and anhydrous DMF (15 mL). The mixture was cooled down in ice. NaH (155 mg of 60% w/w, 3.875 mmol) (60% mineral oil dispersion) was added in one portion. The mixture was stirred under nitrogen at 0° C. for 5 minutes. The ice bath was removed, and the reaction was vigorously stirred under nitrogen for 5 hours. The reaction mixture was slowly poured into an ice-cold citric acid (60 mL of 10% w/v, 31.23 mmol) 10% aqueous solution under stirring. The resulting solid suspension was extracted with EtOAc (3×50 mL). After drying over sodium sulfate, evaporation of the solvents gave a residue that was dissolved in DCM and purified by flash chromatography on silica gel (40 g column) using a gradient of MeOH (0 to 5% over 30 min) in dichloromethane. Evaporation of the solvents and several cycle of trituration/evaporation in DCM/hexanes gave tert-butyl (16S,20S)-12-(2,6-dimethylphenyl)-20-(2-methylpropyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (186 mg, 66%) as a white solid. ESI-MS m/z calc. 635.2778, found 636.4 (M+1)⁺; Retention time: 1.91 minutes (LC method A). ¹H NMR (400 MHz, DMSO-d₆+10% D₂O) two rotamers in a 1:2 ratio. δ 8.32 (s, 0.35H), 8.18 (s, 0.65H), 7.91-7.83 (m, 1H), 7.67 (td, J=7.7, 1.5 Hz, 1H), 7.55 (d, J=7.6 Hz, 1H), 7.29 (t, J=7.6 Hz, 1H), 7.14 (d, J=7.7 Hz, 2H), 6.18 (s, 1H), 5.74-5.51 (m, 1H), 5.12-4.94 (m, 1H), 4.25-3.98 (m, 2H), 3.44 (q, J=14.4 Hz, 1H), 3.22-2.97 (m, 3H), 2.05 (broad s, 6H), 1.69-1.53 (m, 1H), 1.46 (s, 11H), 1.04-0.81 (m, 6H). Sulfonamide NH observed at 13.02 ppm in the absence of D20.

### Step 9: (16R,20S)-12-(2,6-Dimethylphenyl)-20-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 95)

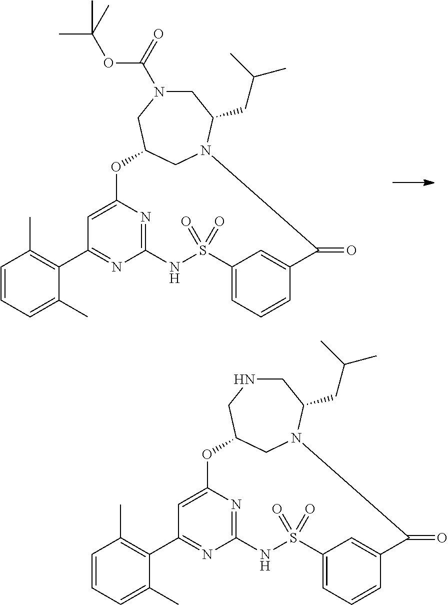 [SMILES: Cc1cccc(C)c1-c1cc2nc(n1)NS(=O)(=O)c1cccc(c1)C(=O)N1C[C@@H](CN(C(=O)OC(C)(C)C)C[C@@H]1CC(C)C)O2 ; Cc1cccc(C)c1-c1cc2nc(n1)NS(=O)(=O)c1cccc(c1)C(=O)N1C[C@@H](CNC[C@@H]1CC(C)C)O2]

A 100 mL flask containing tert-butyl (20S)-12-(2,6-dimethylphenyl)-20-(2-methylpropyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (170 mg, 0.2620 mmol) was charged under nitrogen with DCM (2 mL). HCl (1 mL of 4 M, 4.000 mmol) (4 M dioxane solution) was added and the mixture was stirred at room temperature for 2.5 hours. An additional amount of HCl (0.5 mL of 4 M, 2.000 mmol) was added and the mixture was stirred for an additional 2.5 hours. The volatiles were removed by evaporation and the residue was triturated in DCM/hexanes and the solvents were evaporated. The operation was repeated until a solid was obtained. Drying under vacuum gave (16R,20S)-12-(2,6-dimethylphenyl)-20-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (157 mg, 103%). ESI-MS m/z calc. 535.22534, found 536.45 (M+1)⁺; Retention time: 1.05 minutes (LC method A).

### Example 40: Preparation of Compound 97

### Step 1: (16R,20S)-12-(2,6-Dimethylphenyl)-20-(2-methylpropyl)-18-{spiro[3.4]octan-2-yl}-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 97)

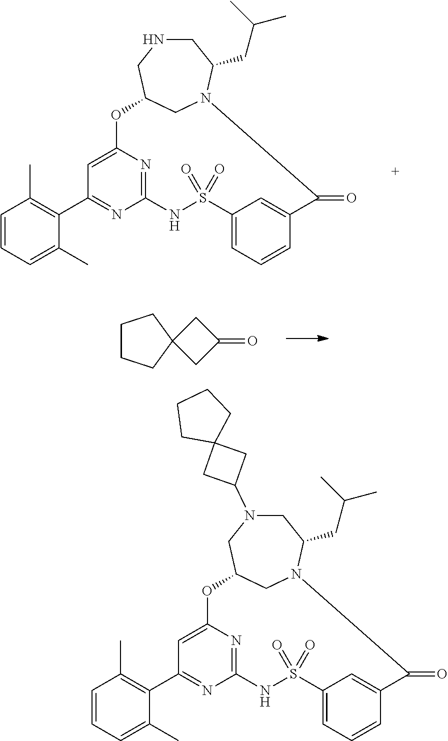

A 4 mL vial was charged with (20S)-12-(2,6-dimethylphenyl)-20-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (20 mg, 0.03426 mmol), spiro[3.4]octan-2-one (34 mg, 0.2738 mmol) anhydrous DCM (400 μL), DIEA (10 μL, 0.05741 mmol) and acetic acid (13 μL, 0.2286 mmol). The vial was capped and stirred at room temperature for about 10 minutes. Sodium triacetoxy borohydride (25 mg, 0.1180 mmol) was added. The vial was purged with nitrogen, capped and the reaction was stirred at room temperature for 4.5 hours. Methanol (100 μL) was added. DCM was evaporated and the residue was taken in DMSO (1 mL). The solution was microfiltered (0.45 m) and purified by reverse phase preparative HPLC (Cis) using a gradient of acetonitrile in water (1 to 99% over 15 min) and HCl as a modifier to give (16R,20S)-12-(2,6-dimethylphenyl)-20-(2-methylpropyl)-18-{spiro[3.4]octan-2-yl}-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (15.3 mg, 64%) as a white solid. ESI-MS m/z calc. 643.3192, found 644.76 (M+1)⁺; Retention time: 1.42 minutes (LC method A).

### Example 41: Preparation of Compound 98

### Step 1: (16R,20S)-12-(2,6-Dimethylphenyl)-20-(2-methylpropyl)-18-(2,2,2-trifluoroethyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 98)

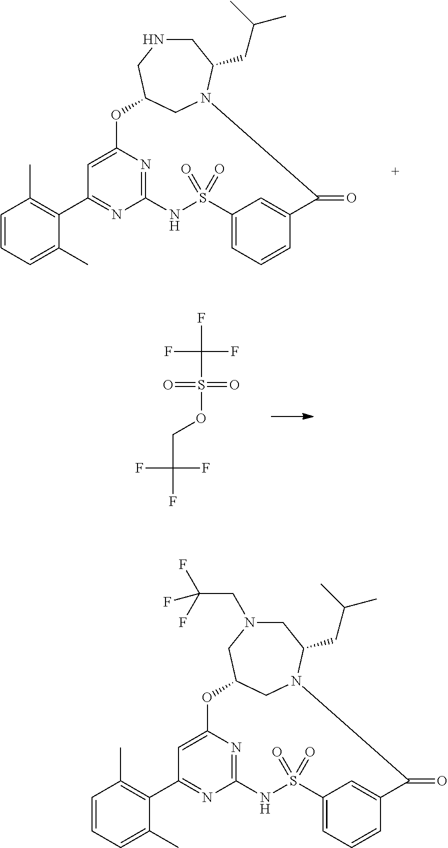 [SMILES: Cc1cccc(C)c1-c1cc2nc(n1)NS(=O)(=O)c1cccc(c1)C(=O)N1C[C@@H](CNC[C@@H]1CC(C)C)O2 ; O=S(=O)(OCC(F)(F)F)C(F)(F)F ; Cc1cccc(C)c1-c1cc2nc(n1)NS(=O)(=O)c1cccc(c1)C(=O)N1C[C@@H](CN(CC(F)(F)F)C[C@@H]1CC(C)C)O2]

A 4 mL vial was charged with (20S)-12-(2,6-dimethylphenyl)-20-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (21 mg, 0.03597 mmol) anhydrous DMF (500 μL), DIEA (19 μL, 0.1091 mmol) and 2,2,2-trifluoroethyl trifluoromethanesulfonate (7 μL, 0.04859 mmol). The vial was purged with nitrogen, capped and the reaction was stirred at room temperature for 2.5 hours. A second amount of reagent 2,2,2-trifluoroethyl trifluoromethanesulfonate (7 μL, 0.04859 mmol) was added and the mixture was stirred for 1.5 h. Methanol (500 μL) was added. The solution was microfiltered and purified by reverse phase preparative HPLC (Cis) using a gradient of acetonitrile in water (1 to 99% over 15 min) and HCl as a modifier. Fractions containing the main peak of the reaction (Rt=0.62 min. using 1 min LCMS method) were collected. Evaporation of the solvents gave (16R,20S)-12-(2,6-dimethylphenyl)-20-(2-methylpropyl)-18-(2,2,2-trifluoroethyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (8.8 mg, 37%) as a white solid. ESI-MS m/z calc. 617.22833, found 618.44 (M+1)⁺; Retention time: 1.52 minutes; LC method A.

### Example 42: Preparation of Compound 99

### Step 1: (16R,20S)-12-(2,6-Dimethylphenyl)-18-methyl-20-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 99)

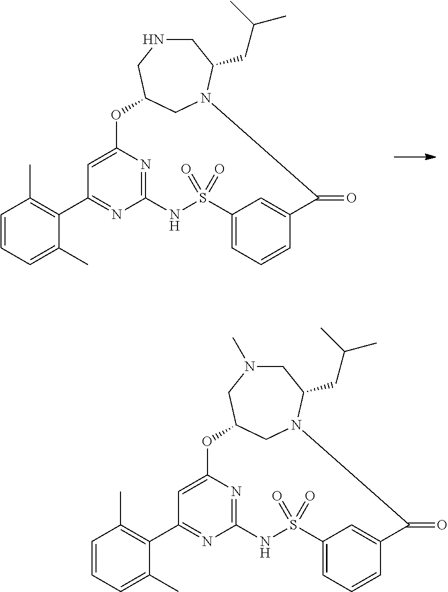 [SMILES: Cc1cccc(C)c1-c1cc2nc(n1)NS(=O)(=O)c1cccc(c1)C(=O)N1C[C@@H](CNC[C@@H]1CC(C)C)O2 ; Cc1cccc(C)c1-c1cc2nc(n1)NS(=O)(=O)c1cccc(c1)C(=O)N1C[C@@H](CN(C)C[C@@H]1CC(C)C)O2]

(16R,20S)-12-(2,6-dimethylphenyl)-20-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (20 mg, 0.03426 mmol) was dissolved in formic acid (250 μL) (88% aqueous) and combined with aqueous formaldehyde (900 μL, 32.67 mmol) (37% aqueous) and heated to 90° C. for 5 hours in a screwcap vial. The reaction mixture was then partially concentrated by blowing nitrogen, diluted with methanol. The solution was microfiltered and purified by reverse phase preparative HPLC (Cis) using a gradient of acetonitrile in water (1 to 99% over 15 min) and HCl as a modifier to give (16R,20S)-12-(2,6-dimethylphenyl)-18-methyl-20-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (13.7 mg, 67%) as a white solid. ESI-MS m/z calc. 549.24097, found 550.43 (M+1)⁺; Retention time: 1.13 minutes; LC method A.

### Example 43: Preparation of Compound 100

### Step 1: (2S,6S)-1-Benzyl-2-isobutyl-1,4-diazepan-6-ol

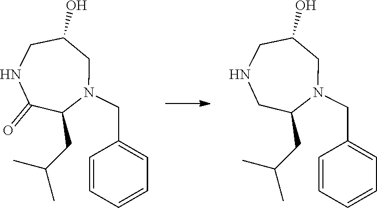 [SMILES: CC(C)C[C@H]1C(=O)NC[C@H](O)CN1Cc1ccccc1 ; CC(C)C[C@H]1CNC[C@H](O)CN1Cc1ccccc1]

Into a solution of (3S,6S)-4-benzyl-6-hydroxy-3-isobutyl-1,4-diazepan-2-one (4.713 g, 17.053 mmol) in anhydrous THE (100 mL) was added LAH (3.8835 g, 102.32 mmol) slowly. The reaction was stirred at 40° C. for 2 days. Water (3.9 mL), 15% NaOH (aqueous) (3.9 mL) and water (11.7 mL) were added to the reaction mixture at 0° C. The reaction mixture was stirred for another 30 minutes, then it was filtered through a pad of Celite. The filter cake was washed with THE (3×20 mL). The combined filtrate was concentrated under vacuum to furnish (2S,6S)-1-benzyl-2-isobutyl-1,4-diazepan-6-ol (4.785 g, 98%) as a clear liquid. The crude product was used directly in the next step reaction without purification. ESI-MS m/z calc. 262.2045, found 263.3 (M+1)⁺; Retention time: 1.95 minutes; LC method S.

### Step 2: tert-Butyl (3S,6R)-4-benzyl-6-hydroxy-3-isobutyl-1,4-diazepane-1-carboxylate

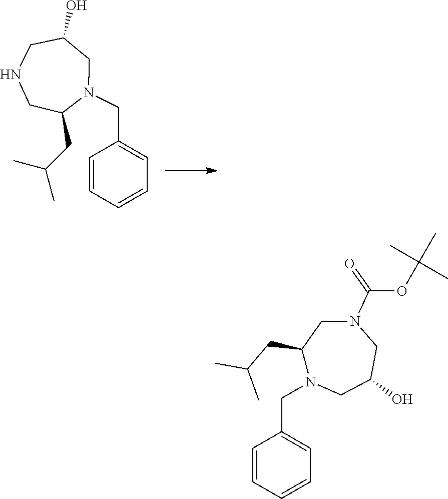 [SMILES: CC(C)C[C@H]1CNC[C@H](O)CN1Cc1ccccc1 ; CC(C)C[C@H]1CN(C(=O)OC(C)(C)C)C[C@H](O)CN1Cc1ccccc1]

Into a solution of (2S,6S)-1-benzyl-2-isobutyl-1,4-diazepan-6-ol (4.785 g, 18.236 mmol) in dichloromethane (50 mL), was added Boc anhydride (5.9699 g, 27.354 mmol) and triethylamine (2.5465 g, 3.5076 mL, 25.166 mmol). The reaction was stirred at room temperature for 2 hours. The reaction was quenched with brine (50 mL). The two layers were separated, and the aqueous layer was extracted with dichloromethane (2×50 mL). The combined organic layers were dried over anhydrous magnesium sulfate and concentrated under vacuum. The residue was purified by silica gel chromatography using 0 to 60% hexane-diethyl ether to furnish tert-butyl (3S,6R)-4-benzyl-6-hydroxy-3-isobutyl-1,4-diazepane-1-carboxylate (3.692 g, 56%) as a clear gel ESI-MS m/z calc. 362.25696, found 363.3 (M+1)⁺; Retention time: 3.84 minutes; LC method S.

### Step 3: tert-Butyl (3S,6R)-6-hydroxy-3-isobutyl-1,4-diazepane-1-carboxylate

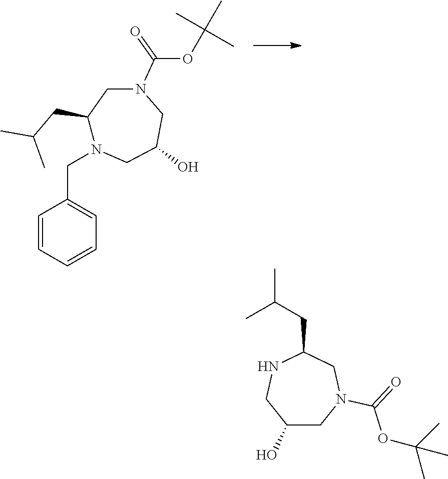 [SMILES: CC(C)C[C@H]1CN(C(=O)OC(C)(C)C)C[C@H](O)CN1Cc1ccccc1 ; CC(C)C[C@H]1CN(C(=O)OC(C)(C)C)C[C@H](O)CN1]

Into a solution of tert-butyl (3S,6R)-4-benzyl-6-hydroxy-3-isobutyl-1,4-diazepane-1-carboxylate (1.919 g, 5.2937 mmol) in methanol (100 mL), was added 10% Palladium on carbon (563 mg). Ammonium formate (1.0014 g, 15.881 mmol) was added to the reaction. The reaction mixture was stirred at 65° C. for 2 h. The catalyst was removed by filtration, and the solution was concentrated under vacuum. The residue was diluted with dichloromethane (100 mL), and washed with water (50 mL) and brine (50 mL), dried over anhydrous sodium sulfate and concentrated under vacuum to furnish tert-butyl (3S,6R)-6-hydroxy-3-isobutyl-1,4-diazepane-1-carboxylate (1.432 g, 94%) as a clear oil. ¹H NMR (250 MHz, DMSO-d₆) δ 4.62 (s, 1H), 3.56 (s, 1H), 3.42 (d, J=6.0 Hz, 1H), 3.26 (dt, J=13.1, 5.6 Hz, 2H), 3.07-2.92 (m, 1H), 2.85-2.58 (m, 2H), 2.30 (dt, J=14.7, 7.6 Hz, 1H), 1.88 (s, 1H), 1.69 (dq, J=13.6, 6.5 Hz, 1H), 1.40 (s, 9H), 1.10 (dq, J=22.6, 6.5, 6.1 Hz, 2H), 0.86 (t, J=6.7 Hz, 6H). ESI-MS m/z calc. 272.21, found 273.1 (M+1)⁺; Retention time: 2.86 minutes; LC method S.

### Step 4: tert-Butyl (3S,6R)-4-[3-[[4-chloro-6-(2,6-dimethylphenyl)pyrimidin-2-yl]sulfamoyl]benzoyl]-6-hydroxy-3-isobutyl-1,4-diazepane-1-carboxylate

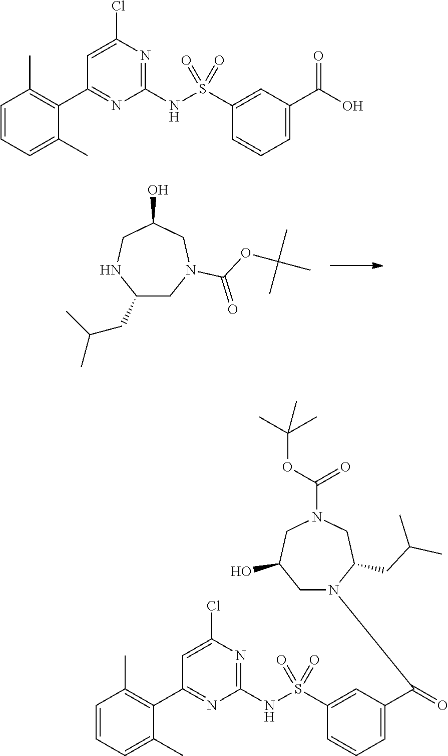 [SMILES: Cc1cccc(C)c1-c1cc(Cl)nc(NS(=O)(=O)c2cccc(C(=O)O)c2)n1 ; CC(C)C[C@H]1CN(C(=O)OC(C)(C)C)C[C@H](O)CN1 ; Cc1cccc(C)c1-c1cc(Cl)nc(NS(=O)(=O)c2cccc(C(=O)N3C[C@@H](O)CN(C(=O)OC(C)(C)C)C[C@@H]3CC(C)C)c2)n1]

A 100 mL flask was charged under nitrogen with tert-butyl (3S,6R)-6-hydroxy-3-isobutyl-1,4-diazepane-1-carboxylate (680 mg, 2.496 mmol), anhydrous DMF (15 mL) and 3-[[4-chloro-6-(2,6-dimethylphenyl)pyrimidin-2-yl]sulfamoyl]benzoic acid (870 mg, 2.082 mmol). After dissolution of the reagents, the mixture was cooled down in an ice bath. DIEA (2.4 mL, 13.78 mmol) and HATU (958 mg, 2.520 mmol) were added and the mixture was stirred at 0° C. for 2.5 hours. The reaction was quenched by being poured in citric acid (75 mL of 10% w/v, 39.04 mmol)(10% aqueous) under vigorous stirring and cooled in ice. The resulting white solid was filtered. The solid was dissolved in DCM and it was purified by flash chromatography on silica gel (120 g column) using a gradient of methanol (0 to 5% over 30 min) in dichloromethane. The product eluted around 2-3% methanol. Evaporation of the solvents gave tert-butyl (3S,6R)-4-[3-[[4-chloro-6-(2,6-dimethylphenyl)pyrimidin-2-yl]sulfamoyl]benzoyl]-6-hydroxy-3-isobutyl-1,4-diazepane-1-carboxylate (1.277 g, 91%) as a white foamy solid. ¹H NMR (400 MHz, DMSO-d₆+10% D₂O) complex mixture of rotamers. δ 8.06-7.95 (m, 1.5H), 7.77 (s, 0.5H), 7.68-7.54 (m, 2H), 7.32-7.21 (m, 2H), 7.14 (dd, J=7.6, 3.0 Hz, 2H), 4.75 (broad s, 0.5H), 4.47 (d, J=13.5 Hz, 0.5H), 3.95 (br d, J=19.9 Hz, 1H), 3.85-3.56 (m, 2H), 3.30 (br s, 1H), 3.27-2.96 (m, 3H), 1.93 (2 singlet, 6H), 1.57 (dt, J=13.3, 6.6 Hz, 1H), 1.44-1.35 (m, 9H), 1.31-1.15 (m, 2H), 1.02-0.91 (m, 3H), 0.62-0.18 (m, 3H). Exchangeable sulfonamide NH visible at 12.34 ppm in the absence of D20. ESI-MS m/z calc. 671.25446, found 672.44 (M+1)⁺; Retention time: 1.98 minutes; LC method A.

### Step 5: tert-Butyl (16R,20S)-12-(2,6-dimethylphenyl)-20-(2-methylpropyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (Compound 100)

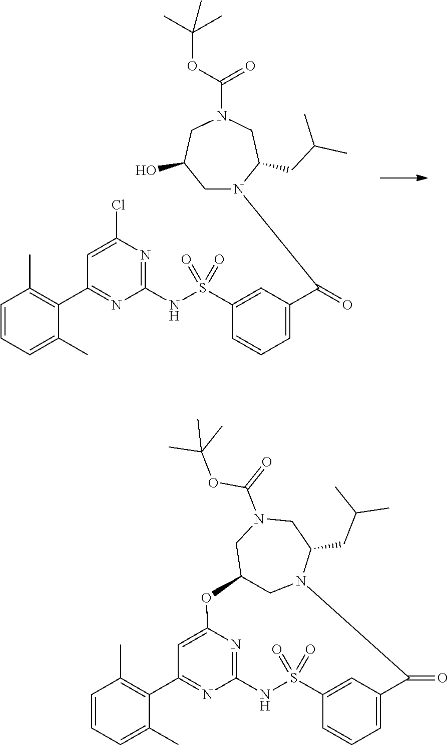 [SMILES: Cc1cccc(C)c1-c1cc(Cl)nc(NS(=O)(=O)c2cccc(C(=O)N3C[C@@H](O)CN(C(=O)OC(C)(C)C)C[C@@H]3CC(C)C)c2)n1 ; Cc1cccc(C)c1-c1cc2nc(n1)NS(=O)(=O)c1cccc(c1)C(=O)N1C[C@H](CN(C(=O)OC(C)(C)C)C[C@@H]1CC(C)C)O2]

A 100 mL flask was charged under nitrogen with tert-butyl (3S)-4-[3-[[4-chloro-6-(2,6-dimethylphenyl)pyrimidin-2-yl]sulfamoyl]benzoyl]-6-hydroxy-3-isobutyl-1,4-diazepane-1-carboxylate (1.258 g, 1.871 mmol) and anhydrous DMF (60 mL). The mixture was cooled down in ice. NaH (665 mg of 60% w/w, 16.63 mmol) (60% mineral oil dispersion) was added in two equal portions. The mixture was stirred under nitrogen at 0° C. for 5 minutes. The ice bath was removed, and the reaction was vigorously stirred under nitrogen for 3 hours. The reaction mixture was slowly poured into an ice-cold citric acid (250 mL of 10% w/v, 130.1 mmol) aqueous solution under stirring. The resulting solid suspension was extracted with EtOAc (3×70 mL). After drying over sodium sulfate, evaporation of the solvents gave a residue that was dissolved in DCM and purified by flash chromatography on silica gel (120 g column) using a gradient of MeOH (0 to 5% over 30 min) in dichloromethane. The product eluted around 3-4% MeOH. Evaporation of the solvents and several cycle of trituration/evaporation in DCM/hexanes gave tert-butyl (16R,20S)-12-(2,6-dimethylphenyl)-20-(2-methylpropyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (420 mg, 35%) as a white solid. ESI-MS m/z calc. 635.2778, found 636.36 (M+1)⁺; Retention time: 1.88 minutes (LC method A). This material was used for the next step without any further purification.

A small amount of material (24 mg) was dissolved in DMSO (1 mL). and purified by reverse phase preparative HPLC (Cis) using a gradient of acetonitrile in water (1 to 99% over 15 min) and HCl as a modifier. The organic solvent was evaporated and the solid that crashed out of water was extracted with DCM (2×20 mL). After drying over sodium sulfate and evaporation, tert-butyl (16R,20S)-12-(2,6-dimethylphenyl)-20-(2-methylpropyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (17 mg, 69%) was isolated as a white solid ¹H NMR (400 MHz, DMSO-d₆+10% D₂O) 2 conformers visible, 1:1 ratio δ 8.19 (d, J=14.2 Hz, 1H), 7.88 (t, J=7.6 Hz, 1H), 7.68 (td, J=7.8, 4.0 Hz, 1H), 7.46 (t, J=7.1 Hz, 1H), 7.28 (t, J=7.6 Hz, 1H), 7.14 (d, J=7.7 Hz, 2H), 6.25 (s, 1H), 5.52-5.32 (m, 1H), 4.29 (t, J=12.4 Hz, 1H),3.95 (signal overlapped with water signal, likely 1H), 3.80-3.56 (m, 2H), 3.33-3.22 (m, 1H), 3.19-2.86 (m, 2H), 2.51-2.39 (m, 1H), 2.04 (broad s, 6H), 1.82-1.66 (m, 1H), 1.48 and 1.43 (two d, 9H), 1.36-1.29 (m, 1H), 0.94 (dd, J=18.0, 6.6 Hz, 6H). sulfonamide NH signal visible around 13.0 ppm in the absence of D20. ESI-MS m/z calc. 635.2778, found 636.43 (M+1)⁺; Retention time: 1.88 minutes; LC method A.

### Example 44: Preparation of Compound 101 and Compound 102

### Step 1: (3R,6R)-4-Benzyl-6-hydroxy-3-isobutyl-1,4-diazepan-2-one

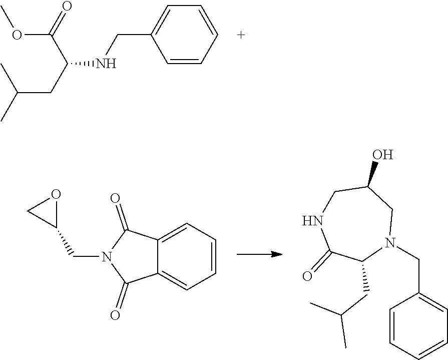 [SMILES: COC(=O)[C@@H](CC(C)C)NCc1ccccc1 ; O=C1c2ccccc2C(=O)N1C[C@@H]1CO1 ; CC(C)C[C@@H]1C(=O)NC[C@@H](O)CN1Cc1ccccc1]

To a solution of 2-[[(2R)-oxiran-2-yl]methyl]isoindoline-1,3-dione (10.422 g, 51.291 mmol) in ACN (72.420 mL) was added methyl (2R)-2-(benzylamino)-4-methyl-pentanoate (12.07 g, 51.291 mmol) and magnesium perchlorate (17.173 g, 76.936 mmol). The reaction mixture was stirred at room temperature overnight before being diluted with water (70 mL) and extracted with DCM (3×75 mL). The combined organic layers were washed with brine (70 mL), dried over anhydrous sodium sulfate and concentrated under vacuum. The organic residue was dissolved in methanol (241.40 mL) and hydrazine hydrate (7.0345 g, 6.8362 mL, 102.58 mmol) was added to the reaction. The reaction was stirred at 65° C. for 24 hours. The reaction was cooled to room temperature and the white solid was filtered off. The filtrate was concentrated and then diluted with 1N NaOH (200 mL) before being extracted with ethyl acetate (3×200 mL). The combined organic layers were washed with brine (200 mL), dried over sodium sulfate and concentrated before being purified by silica gel chromatography eluting 0-5% DCM-MeOH to give (3R,6R)-4-benzyl-6-hydroxy-3-isobutyl-1,4-diazepan-2-one (6.81 g, 45%). ¹H NMR (250 MHz, CDCl₃) δ 7.30 (s, 5H), 5.98 (s, 1H), 3.86 (d, J=14.0 Hz, 2H), 3.72 (dd, J=8.6, 6.2 Hz, 1H), 3.52 (d, J=14.1 Hz, 1H), 3.45-3.32 (m, 2H), 3.19 (dd, J=14.3, 3.8 Hz, 1H), 2.67 (dd, J=14.2, 9.4 Hz, 1H), 1.94-1.77 (m, 1H), 1.77-1.62 (m, 1H), 1.62-1.49 (m, 1H), 0.95 (dd, J=6.6, 5.5 Hz, 6H). ESI-MS m/z calc. 276.18378, found 277.1 (M+1)⁺; Retention time: 2.73 minutes; LC method S.

### Step 2: (2R,6R)-1-Benzyl-2-isobutyl-1,4-diazepan-6-ol

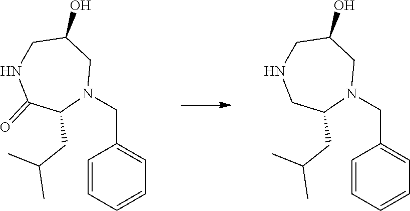 [SMILES: CC(C)C[C@@H]1C(=O)NC[C@@H](O)CN1Cc1ccccc1 ; CC(C)C[C@@H]1CNC[C@@H](O)CN1Cc1ccccc1]

Into a solution of (3R,6R)-4-benzyl-6-hydroxy-3-isobutyl-1,4-diazepan-2-one (6.81 g, 24.641 mmol) in THE (120 mL) was added LAH (5.6115 g, 147.85 mmol). The reaction was stirred at 40° C. overnight before being cooled to room temperature. The reaction was quenched with water (5.6 mL), 15% NaOH (aqueous) (5.6 mL) and water (16.8 mL) at 0° C. subsequently. The reaction mixture was stirred for another 30 minutes, then it was filtered through a pad of Celite. The filter cake was washed with THE (3×50 mL). The combined filtrate was concentrated under vacuum to give (2R,6R)-1-benzyl-2-isobutyl-1,4-diazepan-6-ol (6.53 g, 101%). ESI-MS m/z calc. 262.2045, found 263.3 (M+1)⁺; Retention time: 2.38 minutes; LC method S.

### Step 3: tert-Butyl (3R,6S)-4-benzyl-6-hydroxy-3-isobutyl-1,4-diazepane-1-carboxylate

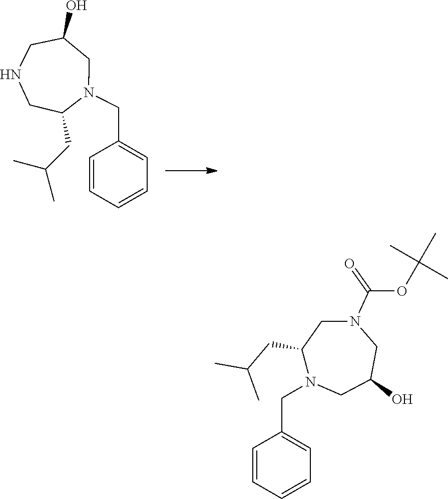 [SMILES: CC(C)C[C@@H]1CNC[C@@H](O)CN1Cc1ccccc1 ; CC(C)C[C@@H]1CN(C(=O)OC(C)(C)C)C[C@@H](O)CN1Cc1ccccc1]

To a solution of (2R,6R)-1-benzyl-2-isobutyl-1,4-diazepan-6-ol (6.53 g, 24.887 mmol) in DCM (78.360 mL) was added Boc anhydride (8.1474 g, 37.331 mmol) and triethylamine (3.7775 g, 5.2032 mL, 37.331 mmol) at room temperature and stirred overnight. The reaction was quenched with brine (100 mL). The two layers were separated, and the aqueous layer was extracted with DCM (2×100 mL). The combined organic layers were dried over anhydrous magnesium sulfate and concentrated under vacuum. The residue was purified by silica gel chromatography using 0-5% DCM-MeOH to give tert-butyl (3R,6S)-4-benzyl-6-hydroxy-3-isobutyl-1,4-diazepane-1-carboxylate (7.02 g, 78%). ESI-MS m/z calc. 362.25696, found 363.2 (M+1)⁺; Retention time: 3.68 minutes; LC method S.

### Step 4: tert-Butyl (3R,6S)-6-hydroxy-3-isobutyl-1,4-diazepane-1-carboxylate

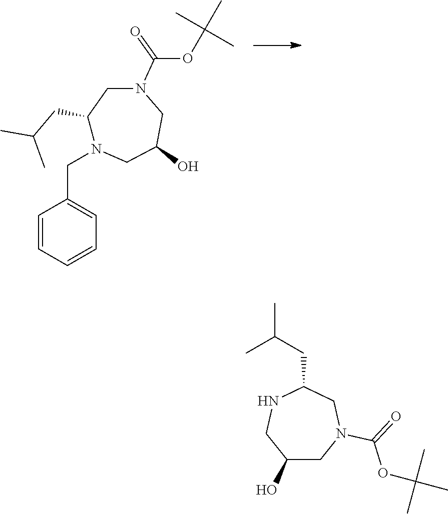 [SMILES: CC(C)C[C@@H]1CN(C(=O)OC(C)(C)C)C[C@@H](O)CN1Cc1ccccc1 ; CC(C)C[C@@H]1CN(C(=O)OC(C)(C)C)C[C@@H](O)CN1]

To a solution of tert-butyl (3R,6S)-4-benzyl-6-hydroxy-3-isobutyl-1,4-diazepane-1-carboxylate (3.51 g, 9.6826 mmol) in methanol (100 mL) was added Palladium (1.0305 g, 0.9683 mmol) and ammonium formate (1.8316 g, 29.048 mmol). The reaction was stirred at 65° C. for 2 hour. Palladium was removed by filtration, and the solution was concentrated under vacuum. The residue was diluted with dichloromethane (100 mL), and washed with water (50 mL) and brine (50 mL), dried over anhydrous sodium sulfate and concentrated under vacuum to give tert-butyl (3R,6S)-6-hydroxy-3-isobutyl-1,4-diazepane-1-carboxylate (2.41 g, 91%)¹H NMR (250 MHz, DMSO-d₆) δ 4.61 (s, 1H), 3.63-3.49 (m, 1H), 3.46-3.39 (m, 1H), 3.32-3.20 (m, 2H), 3.07-2.92 (m, 1H), 2.87-2.59 (m, 2H), 2.38-2.22 (m, 1H), 1.77-1.60 (m, 1H), 1.39 (s, 9H), 1.22-0.97 (m, 2H), 0.86 (t, J=6.7 Hz, 6H). ESI-MS m/z calc. 272.21, found 273.1 (M+1)⁺; Retention time: 2.77 minutes; LC method S.

### Step 5: tert-Butyl (3R,6S)-4-[3-[[4-chloro-6-(2,6-dimethylphenyl)pyrimidin-2-yl]sulfamoyl]benzoyl]-6-hydroxy-3-isobutyl-1,4-diazepane-1-carboxylate

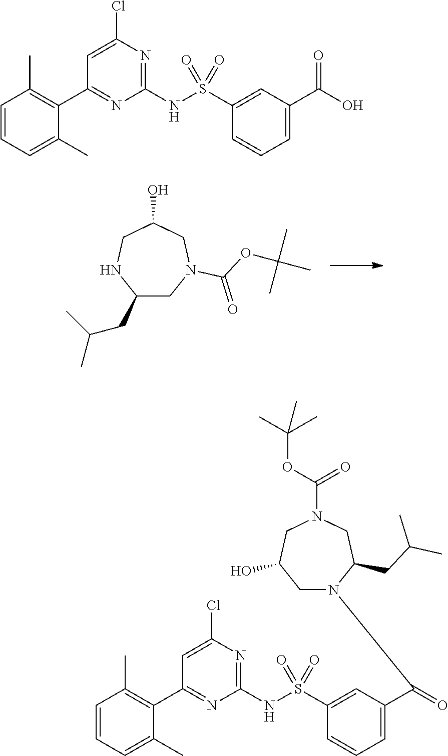 [SMILES: Cc1cccc(C)c1-c1cc(Cl)nc(NS(=O)(=O)c2cccc(C(=O)O)c2)n1 ; CC(C)C[C@@H]1CN(C(=O)OC(C)(C)C)C[C@@H](O)CN1 ; Cc1cccc(C)c1-c1cc(Cl)nc(NS(=O)(=O)c2cccc(C(=O)N3C[C@H](O)CN(C(=O)OC(C)(C)C)C[C@H]3CC(C)C)c2)n1]

A 100 mL flask was charged under nitrogen with tert-butyl (3R,6S)-6-hydroxy-3-isobutyl-1,4-diazepane-1-carboxylate (998 mg, 3.664 mmol), anhydrous DMF (20 mL) and 3-[[4-chloro-6-(2,6-dimethylphenyl)pyrimidin-2-yl]sulfamoyl]benzoic acid (1.279 g, 3.061 mmol). After dissolution of the reagents, the mixture was cooled down in an ice bath. DIEA (3.6 mL, 20.67 mmol) and HATU (1.43 g, 3.761 mmol) were added and the mixture was stirred at 0° C. for 3.5 hours. The reaction was quenched by being poured in citric acid (110 mL of 10% w/v, 57.25 mmol)(10% aqueous) under vigorous stirring and cooled in ice. The resulting white solid was filtered. The wet solid was dissolved in DCM and the solution was dried over sodium sulfate. After concentration, it was purified by flash chromatography on silica gel (120 g column) using a gradient of methanol (0 to 5% over 30 min) in dichloromethane. The product eluted around 3-4% methanol. Evaporation of the solvents gave tert-butyl (3R,6S)-4-[3-[[4-chloro-6-(2,6-dimethylphenyl)pyrimidin-2-yl]sulfamoyl]benzoyl]-6-hydroxy-3-isobutyl-1,4-diazepane-1-carboxylate (1.783 g, 87%) as a white foamy solid. ESI-MS m/z calc. 671.25446, found 672.33 (M+1)⁺; Retention time: 1.92 minutes; LC method A.

### Step 6: tert-Butyl (16S,20R)-12-(2,6-dimethylphenyl)-20-(2-methylpropyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (Compound 102)

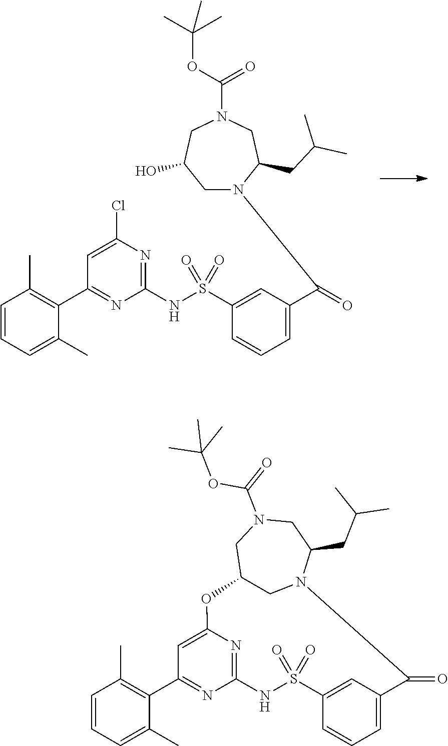 [SMILES: Cc1cccc(C)c1-c1cc(Cl)nc(NS(=O)(=O)c2cccc(C(=O)N3C[C@H](O)CN(C(=O)OC(C)(C)C)C[C@H]3CC(C)C)c2)n1 ; Cc1cccc(C)c1-c1cc2nc(n1)NS(=O)(=O)c1cccc(c1)C(=O)N1C[C@@H](CN(C(=O)OC(C)(C)C)C[C@H]1CC(C)C)O2]

A 100 mL flask was charged under nitrogen with tert-butyl (3R,6S)-4-[3-[[4-chloro-6-(2,6-dimethylphenyl)pyrimidin-2-yl]sulfamoyl]benzoyl]-6-hydroxy-3-isobutyl-1,4-diazepane-1-carboxylate (1.45 g, 2.157 mmol) and anhydrous DMF (70 mL). The mixture was cooled down in ice. NaH (768 mg of 60% w/w, 19.20 mmol) (60% mineral oil dispersion) was added in two equal portions, added 3 minutes after each other. The mixture was stirred under nitrogen at 0° C. for 10 minutes. The ice bath was removed, and the reaction was vigorously stirred under nitrogen for 4 hours. The reaction mixture was slowly poured into an ice-cold citric acid (300 mL of 10% w/v, 156.1 mmol) aqueous solution under stirring. The resulting solid suspension was extracted with EtOAc (3×75 mL). After drying over sodium sulfate, evaporation of the solvents gave a residue that was dissolved in DCM and purified by flash chromatography on silica gel (120 g column) using a gradient of MeOH (0 to 5% over 30 min) in dichloromethane. The product eluted around 3-4% MeOH. Evaporation of the solvents and several cycles of trituration/evaporation in DCM/hexanes gave tert-butyl (16S,20R)-12-(2,6-dimethylphenyl)-20-(2-methylpropyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (481 mg, 34%) as an off-white solid. ¹H NMR (500 MHz, DMSO-d₆+10% D₂O) two rotamers visible in a (60:40) ratio δ 8.18 and 8.15 (two s, 60:40 ratio, total 1H), 7.85 (t, J=8.6 Hz, 1H), 7.73-7.59 (m, 1H), 7.49-7.39 (m, 1H), 7.25 (t, J=7.7 Hz, 1H), 7.11 (d, J=7.8 Hz, 2H), 6.26 (s, 1H), 5.40 (broad s, 1H), 4.26 (t, J=12.1 Hz, 1H), 3.97-3.84 (m, 1H), 3.70-3.55 (m, 2H overlapped with water signal), 3.24 (br s, 1H), 3.16-2.89 (m, 2H), 2.46-2.34 (m, 1H), 2.01 (br s, 6H), 1.76-1.61 (m, 1H), 1.51-1.23 (m, 10H), 0.92 (dd, J=21.8, 6.6 Hz, 6H). ESI-MS m/z calc. 635.2778, found 636.32 (M+1)⁺; Retention time: 1.93 minutes; LC method A.

### Step 7: (16R,20R)-12-(2,6-Dimethylphenyl)-20-(2-methylpropyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione (Compound 101)

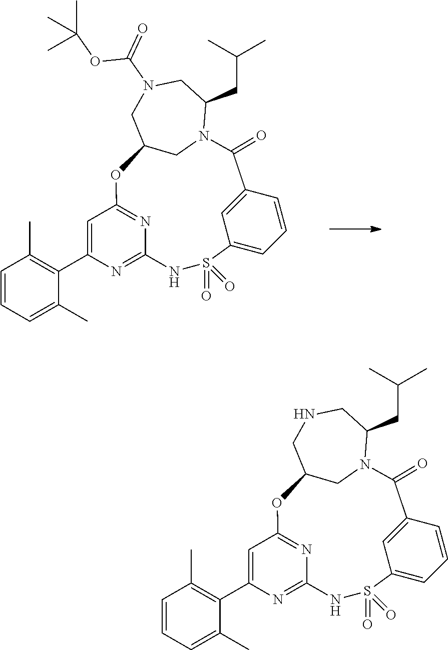 [SMILES: Cc1cccc(C)c1-c1cc2nc(n1)NS(=O)(=O)c1cccc(c1)C(=O)N1C[C@H](CN(C(=O)OC(C)(C)C)C[C@H]1CC(C)C)O2 ; Cc1cccc(C)c1-c1cc2nc(n1)NS(=O)(=O)c1cccc(c1)C(=O)N1C[C@H](CNC[C@H]1CC(C)C)O2]

A 100 mL flask containing tert-butyl (16S,20R)-12-(2,6-dimethylphenyl)-20-(2-methylpropyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (445 mg, 0.6859 mmol) was charged under nitrogen with DCM (15 mL). 4 M hydrogen chloride in dioxane (4.3 mL of 4.0 M, 17.20 mmol) (4 M dioxane solution) was added and the mixture was stirred at room temperature for 2.5 hours (94% conversion). An additional amount of was added and the mixture was stirred for an additional 2.5 hours. The volatiles were removed under reduced pressure and the residue was triturated with dichloromethane/hexanes and the volatiles were evaporated. The operation was repeated until a solid was obtained. Drying under vacuum gave (16R,20R)-12-(2,6-dimethylphenyl)-20-(2-methylpropyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione (hydrochloride salt) (393 mg, 100%). ESI-MS m/z calc. 535.22534, found 536.4 (M+1)⁺; Retention time: 1.17 minutes; LC method A.

### Example 45: Preparation of Compound 103

### Step 1: (16R,20R)-12-(2,6-Dimethylphenyl)-18-methyl-20-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 103)

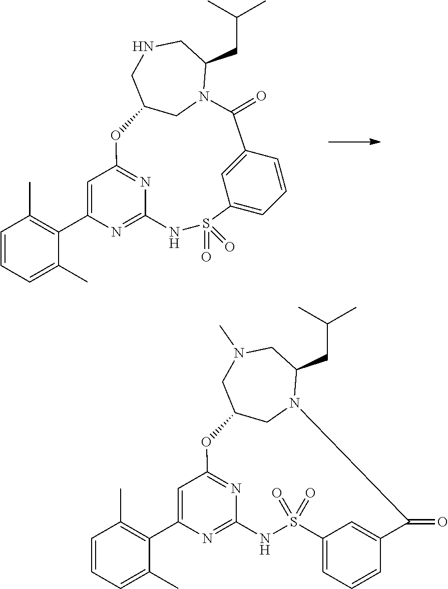 [SMILES: Cc1cccc(C)c1-c1cc2nc(n1)NS(=O)(=O)c1cccc(c1)C(=O)N1C[C@@H](CNC[C@H]1CC(C)C)O2 ; Cc1cccc(C)c1-c1cc2nc(n1)NS(=O)(=O)c1cccc(c1)C(=O)N1C[C@@H](CN(C)C[C@H]1CC(C)C)O2]

(16R,20R)-12-(2,6-dimethylphenyl)-20-(2-methylpropyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione (hydrochloride salt) (25 mg, 0.04370 mmol) was dissolved in formic acid (250 μL) (88% aqueous) and combined with aqueous formaldehyde (900 μL, 32.67 mmol) (37% aqueous) and heated to 90° C. for 4 hours in a screwcap vial. The reaction mixture was then partially concentrated by blowing nitrogen, diluted with methanol. The solution was microfiltered and purified by reverse phase preparative HPLC (Cis) using a gradient of acetonitrile in water (1 to 99% over 15 min) and HCl as a modifier to give (16R,20R)-12-(2,6-dimethylphenyl)-18-methyl-20-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (15.7 mg, 60%) as a white solid. ESI-MS m/z calc. 549.24097, found 550.68 (M+1)⁺; Retention time: 1.12 minutes; LC method A.

### Example 46: Preparation of Compound 104

### Step 1: (16R,20R)-12-(2,6-Dimethylphenyl)-18-(2,2-dimethylpropyl)-20-(2-methylpropyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione (Compound 104)

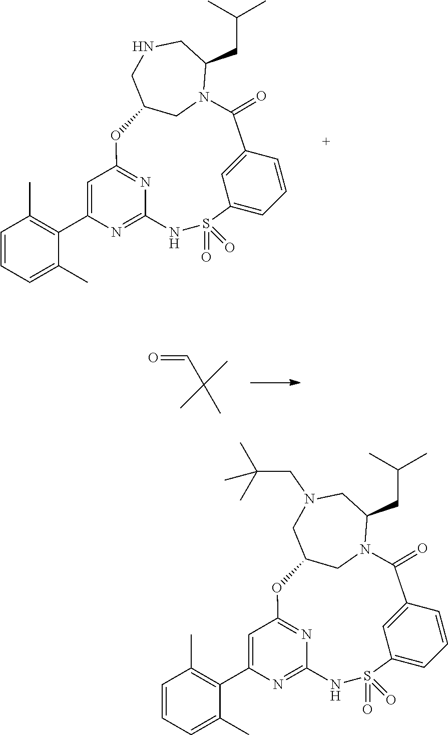

A 4 mL vial was charged with (16R,20R)-12-(2,6-dimethylphenyl)-20-(2-methylpropyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione (hydrochloride salt) (20 mg, 0.03496 mmol), 2,2-dimethylpropanal (30 mg, 0.3483 mmol), anhydrous dichloromethane (1 mL), N,N-diisopropyl ethyl amine (10 μL, 0.05741 mmol) and glacial acetic acid (10 μL, 0.1758 mmol), in that order. The vial was capped under nitrogen and stirred at room temperature for 30 min. Then sodium triacetoxyborohydride (40 mg, 0.1887 mmol) was added at once. The vial was purged with nitrogen, capped and the reaction mixture was stirred at room temperature for 13 h. Methanol (0.2 mL) was added and the volatiles were removed under reduced pressure. The residue was taken up in DMSO (1 mL). The solution was microfiltered and purified by reverse-phase preparative HPLC (Cis) using a gradient of acetonitrile in water (1 to 99% over 15 min) and HCl as a modifier to give (16R,20R)-12-(2,6-dimethylphenyl)-18-(2,2-dimethylpropyl)-20-(2-methylpropyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione (hydrochloride salt) (11 mg, 49%) as a white solid. ESI-MS m/z calc. 605.3036, found 606.4 (M+1)⁺; Retention time: 1.95 minutes; LC method A.

### Example 47: Preparation of Compound 105

### Step 1: (16R,20R)-12-(2,6-Dimethylphenyl)-20-(2-methylpropyl)-18-(2,2,2-trifluoroethyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione (Compound 105)

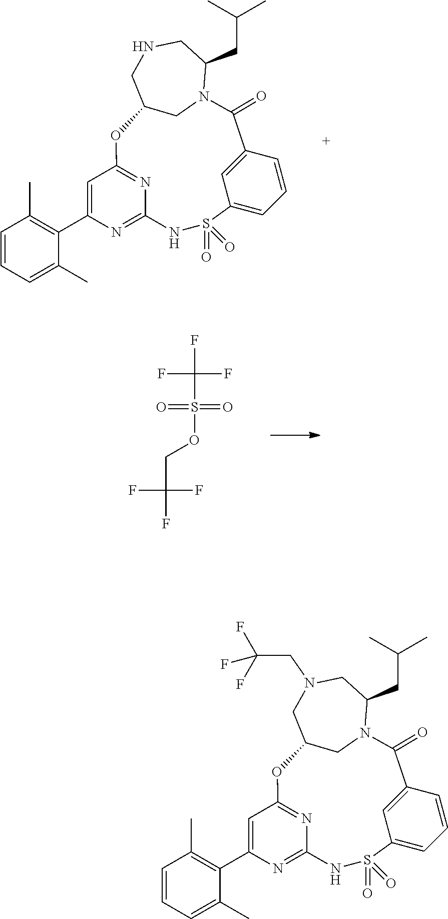 [SMILES: Cc1cccc(C)c1-c1cc2nc(n1)NS(=O)(=O)c1cccc(c1)C(=O)N1C[C@@H](CNC[C@H]1CC(C)C)O2 ; O=S(=O)(OCC(F)(F)F)C(F)(F)F ; Cc1cccc(C)c1-c1cc2nc(n1)NS(=O)(=O)c1cccc(c1)C(=O)N1C[C@@H](CN(CC(F)(F)F)C[C@H]1CC(C)C)O2]

To a stirred solution of 2,2,2-trifluoroethyl trifluoromethanesulfonate (7.5 mg, 0.03231 mmol) and (16R,20R)-12-(2,6-dimethylphenyl)-20-(2-methylpropyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione (hydrochloride salt) (14 mg, 0.02447 mmol) in anhydrous DMF (0.6 mL) was added DIEA (15 μL, 0.08612 mmol) and purged with nitrogen for 30 sec. The clear reaction was stirred at ambient temperature for 2 h. The reaction was microfiltered and purified from reverse-phase column chromatography (C₁₈ column), eluting with 1-9 acetonitrile/water over 15 min (5 mM HCl in water as modifier). The desired fractions were concentrated and dried to furnish (16R,20R)-12-(2,6-dimethylphenyl)-20-(2-methylpropyl)-18-(2,2,2-trifluoroethyl)-15-oxa-8λ⁶-thia-1,9,11,18,22-pentaazatetracyclo[14.4.1.13,7.110,14]tricosa-3(23),4,6,10,12,14(22)-hexaene-2,8,8-trione (8.1 mg, 53%) as a white solid. ¹H NMR (499 MHz, DMSO-d₆) δ 9.81 (s, 1H), 9.03 (t, J=1.7 Hz, 1H), 8.08 (d, J=8.0 Hz, 1H), 7.78 (t, J=7.8 Hz, 1H), 7.69 (dt, J=7.8, 1.2 Hz, 1H), 7.22 (t, J=7.6 Hz, 1H), 7.11 (d, J=7.6 Hz, 2H), 6.66 (s, 1H), 5.57-5.43 (m, 1H), 5.20 (dq, J=17.5, 8.8 Hz, 1H), 4.99 (dq, J=17.1, 8.6 Hz, 1H), 4.01-3.85 (m, 2H), 3.71 (d, J=13.1 Hz, 1H), 3.58-3.45 (m, 3H), 3.22 (d, J=12.7 Hz, 1H), 3.09 (dd, J=14.2, 10.8 Hz, 1H), 1.96 (s, 6H), 1.68 (dq, J=13.0, 6.4 Hz, 1H), 1.35 (ddd, J=13.7, 9.1, 4.8 Hz, 1H), 0.96 (d, J=6.6 Hz, 3H), 0.91 (d, J=6.6 Hz, 3H). ESI-MS m/z calc. 617.22833, found 618.4 (M+1)⁺; Retention time: 1.72 minutes; LC method A.

### Example 48: Preparation of Compound 106

### Step 1: Methyl (2R)-2-(benzylamino)-4-methyl-pentanoate

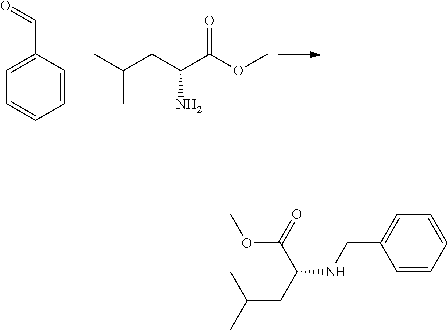

To a solution of methyl (2R)-2-amino-4-methyl-pentanoate (hydrochloride salt) (30 g, 161.84 mmol) in Methanol (300.00 mL) was added TEA (16.377 g, 22.558 mL, 161.84 mmol), and Magnesium sulfate (32.368 g, 268.91 mmol) at room temperature and stirred for 10 minutes. benzaldehyde (17.175 g, 16.357 mL, 161.84 mmol) was added to the mixture and was stirred for 2 days at room temperature. The solution was filtered through a pad of Celite and Sodium borohydride (12.246 g, 12.959 mL, 323.68 mmol) was slowly added while in an ice bath. The reaction was stirred for 1 hour before being quenched with ammonium chloride (150 mL). Methanol was removed in vacuum and the solution was extracted with diethyl ether (3×300 mL). The organic layers were washed with water (300 mL) and brine (300 mL) then dried over sodium sulfate and concentrated before being purified by silica gel chromatography eluting 0-20% hexanes-ethyl acetate to yield methyl (2R)-2-(benzylamino)-4-methyl-pentanoate (26.8 g, 70%) as a clear liquid. ¹H NMR (250 MHz, CDCl₃) δ 7.47-6.90 (m, 5H), 3.81 (d, J=12.9 Hz, 1H), 3.72 (s, 3H), 3.61 (d, J=12.9 Hz, 1H), 3.31 (t, J=7.2 Hz, 1H), 1.83-1.71 (m, 1H), 1.66 (s, 1H), 1.48 (dd, J=7.6, 6.3 Hz, 2H), 0.88 (dd, J=16.4, 6.6 Hz, 6H). ESI-MS m/z calc. 235.15723, found 236.3 (M+1)⁺; Retention time: 2.66 minutes; LC method S.

### Step 2: (3R,6S)-4-Benzyl-6-hydroxy-3-isobutyl-1,4-diazepan-2-one

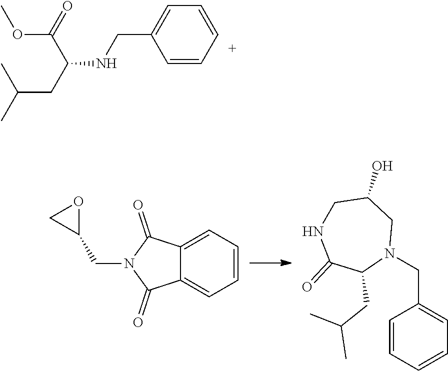 [SMILES: COC(=O)[C@@H](CC(C)C)NCc1ccccc1 ; O=C1c2ccccc2C(=O)N1C[C@@H]1CO1 ; CC(C)C[C@@H]1C(=O)NC[C@H](O)CN1Cc1ccccc1]

To a stirring solution of 2-[[(2R)-oxiran-2-yl]methyl]isoindoline-1,3-dione (10.880 g, 53.544 mmol) and methyl (2R)-2-(benzylamino)-4-methyl-pentanoate (12 g, 50.994 mmol) in acetonitrile (75 mL) at room temperature was portionwise added magnesium perchlorate (17.073 g, 76.491 mmol). After the addition was complete, the reaction mixture was stirred for 24 hours. The reaction was quenched with water (300 mL) and the product was extracted with DCM (3×200 mL). The combined organic layers were washed with brine (80 mL), dried over anhydrous sodium sulfate and concentrated. The obtained residue was dissolved in methanol (250 mL) at room temperature, and hydrazine hydrate (5.1057 g, 101.99 mmol) was added. The reaction mixture was heated to 65° C. for 24 hours. After cooling to room temperature, the white precipitate was filtered off and the filtrate was concentrated under vacuum. The obtained residue was diluted with 1 M aqueous NaOH (200 mL) and the product was extracted with ethyl acetate (3×200 mL). The combined organic layers were washed with brine (100 mL), dried over anhydrous sodium sulfate and concentrated. The crude was purified by silica gel chromatography using 0-5% DCM-methanol to afford as a white foam (3R,6S)-4-benzyl-6-hydroxy-3-isobutyl-1,4-diazepan-2-one (8.43 g, 57%) ¹H NMR (250 MHz, CDCl₃) δ 7.38-7.14 (m, 5H), 6.08 (s, 1H), 3.96-3.71 (m, 3H), 3.67-3.43 (m, 2H), 3.42-3.23 (m, 1H), 3.21-3.02 (m, 1H), 2.98-2.82 (m, 1H), 1.98-1.66 (m, 3H), 1.62-1.44 (m, 1H), 1.03-0.79 (m, 6H). ESI-MS m/z calc. 276.18378, found 277.7 (M+1)⁺; Retention time: 2.64 minutes; LC method S.

### Step 3: tert-Butyl (3R,6R)-4-benzyl-6-hydroxy-3-isobutyl-1,4-diazepane-1-carboxylate

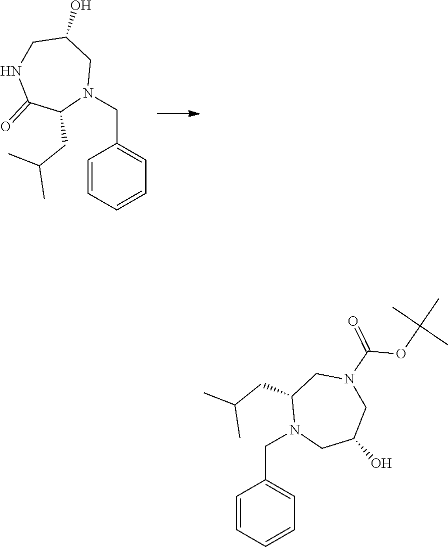 [SMILES: CC(C)C[C@@H]1C(=O)NC[C@H](O)CN1Cc1ccccc1 ; CC(C)C[C@@H]1CN(C(=O)OC(C)(C)C)C[C@H](O)CN1Cc1ccccc1]

To a stirring solution of (3R,6S)-4-benzyl-6-hydroxy-3-isobutyl-1,4-diazepan-2-one (8.43 g, 30.502 mmol) in anhydrous THE (260 mL) at 0° C. under nitrogen was added portionwise LAH (6.9460 g, 183.01 mmol). After the addition was complete, the reaction mixture was stirred at 0° C. for 10 minutes, and then heated to 45° C. for 24 hours. The reaction mixture was cooled to 0° C. and then quenched following a Fieser workup procedure. Salts were filtered off through a pad of Celite, the filter cake was washed with THE (2×150 mL), and the combined filtrate was concentrated under vacuum. The obtained residue was dissolved in DCM (90 mL) and cooled to 0° C. TEA (4.6297 g, 6.3770 mL, 45.753 mmol) was added, followed by Boc anhydride (9.9855 g, 45.753 mmol). The reaction mixture was stirred at 0° C. for 1 hour. The reaction was quenched cold with brine (200 mL) and 2 layers were separated. The aqueous layer was extracted with DCM (2×150 mL). The combined organic layers were dried over anhydrous sodium sulfate and concentrated. The crude was purified by silica gel chromatography using 0-25% hexanes-acetone to afford tert-butyl (3R,6R)-4-benzyl-6-hydroxy-3-isobutyl-1,4-diazepane-1-carboxylate (11.54 g, 99%) as a yellow oil. ¹H NMR (250 MHz, CDCl₃) δ 7.39-7.14 (m, 5H), 3.98-3.60 (m, 3H), 3.57-3.19 (m, 3H), 2.97-2.56 (m, 3H), 1.83-1.57 (m, 1H), 1.56-1.18 (m, 13H), 1.01-0.76 (m, 6H). ESI-MS m/z calc. 362.25696, found 363.7 (M+1)⁺; Retention time: 4.19 minutes; LC method S.

### Step 4: tert-Butyl (3R,6R)-6-hydroxy-3-isobutyl-1,4-diazepane-1-carboxylate

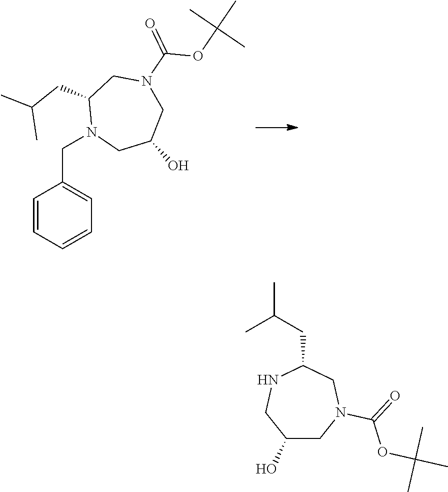 [SMILES: CC(C)C[C@@H]1CN(C(=O)OC(C)(C)C)C[C@H](O)CN1Cc1ccccc1 ; CC(C)C[C@@H]1CN(C(=O)OC(C)(C)C)C[C@H](O)CN1]

To a stirring solution of tert-butyl (3R,6R)-4-benzyl-6-hydroxy-3-isobutyl-1,4-diazepane-1-carboxylate (5.3 g, 14.620 mmol) in anhydrous methanol (140 mL) at room temperature was added palladium on carbon (2.3338 g, 10% w/w, 2.1930 mmol), followed by ammonium formate (3.6875 g, 58.480 mmol). The reaction mixture was heated to 65° C. for 1 hour. After cooling to room temperature, the reaction mixture was filtered through a pad of Celite and the filter cake was washed with methanol (2×80 mL). The combined filtrate was concentrated under vacuum to afford tert-butyl (3R,6R)-6-hydroxy-3-isobutyl-1,4-diazepane-1-carboxylate (3.98 g, 92%) as a colorless oil. ¹H NMR (250 MHz, DMSO-d₆) δ 4.68 (s, 1H), 4.13 (s, 1H), 3.93-3.63 (m, 3H), 2.98-2.53 (m, 3H), 2.45-2.30 (m, 1H), 2.05-1.56 (m, 1H), 1.42-1.32 (m, 9H), 1.27-0.95 (m, 3H), 0.94-0.75 (m, 6H). ESI-MS m/z calc. 272.21, found 273.3 (M+1)⁺; Retention time: 1.61 minutes; LC method T.

### Step 5: tert-Butyl (3R,6R)-4-[3-[[4-chloro-6-(2,6-dimethylphenyl)pyrimidin-2-yl]sulfamoyl]benzoyl]-6-hydroxy-3-isobutyl-1,4-diazepane-1-carboxylate

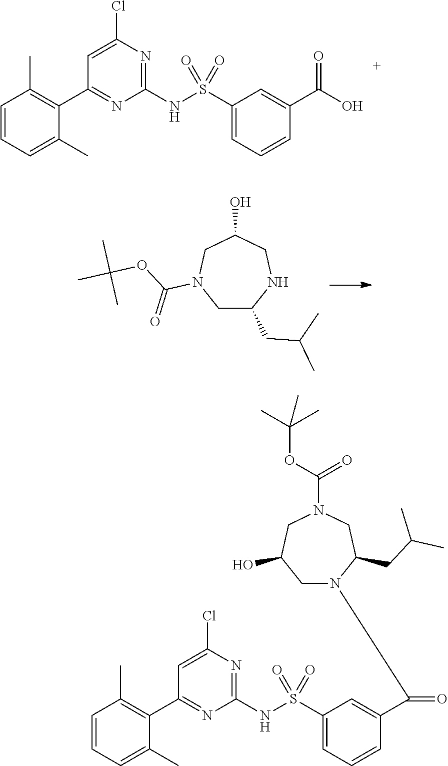

A 100 mL flask was charged under nitrogen with tert-butyl (3R,6R)-6-hydroxy-3-isobutyl-1,4-diazepane-1-carboxylate (933 mg, 3.425 mmol), anhydrous DMF (20 mL) and 3-[[4-chloro-6-(2,6-dimethylphenyl)pyrimidin-2-yl]sulfamoyl]benzoic acid (1.19 g, 2.848 mmol). After dissolution of the reagents, the mixture was cooled down in an ice bath. DIEA (3.3 mL, 18.95 mmol) and HATU (1.33 g, 3.498 mmol) were added and the mixture was stirred at 0° C. for 5 hours. The reaction was quenched by being poured in citric acid (100 mL of 10% w/v, 52.05 mmol)(10% aqueous) under vigorous stirring and cooled in ice. The resulting white solid was filtered and dried. The solid was dissolved in DCM and it was purified by flash chromatography on silica gel (120 g column) using a gradient of methanol (0 to 5% over 30 min) in dichloromethane. The product eluted around 3-4% methanol. Evaporation of the solvents gave tert-butyl (3R,6R)-4-[3-[[4-chloro-6-(2,6-dimethylphenyl)pyrimidin-2-yl]sulfamoyl]benzoyl]-6-hydroxy-3-isobutyl-1,4-diazepane-1-carboxylate (1.183 g, 62%) as an off-white foamy solid. ESI-MS m/z calc. 671.25446, found 672.26 (M+1)⁺; Retention time: 1.98 minutes; LC method A.

### Step 6: tert-Butyl (16R,20R)-12-(2,6-dimethylphenyl)-20-(2-methylpropyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (Compound 106)

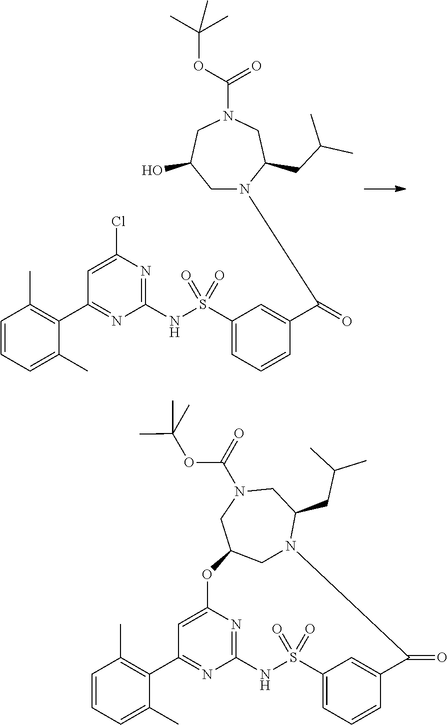 [SMILES: Cc1cccc(C)c1-c1cc(Cl)nc(NS(=O)(=O)c2cccc(C(=O)N3C[C@@H](O)CN(C(=O)OC(C)(C)C)C[C@H]3CC(C)C)c2)n1 ; Cc1cccc(C)c1-c1cc2nc(n1)NS(=O)(=O)c1cccc(c1)C(=O)N1C[C@H](CN(C(=O)OC(C)(C)C)C[C@H]1CC(C)C)O2]

A 100 mL flask was charged under nitrogen with tert-butyl (3R,6R)-4-[3-[[4-chloro-6-(2,6-dimethylphenyl)pyrimidin-2-yl]sulfamoyl]benzoyl]-6-hydroxy-3-isobutyl-1,4-diazepane-1-carboxylate (1.158 g, 1.723 mmol) and anhydrous DMF (55 mL). The mixture was cooled down in ice. NaH (604 mg of 60% w/w, 15.10 mmol) (60% mineral oil dispersion) was added in two equal portions, added 3 minutes after each other. The mixture was stirred under nitrogen at 0° C. for 10 minutes. The ice bath was removed, and the reaction was vigorously stirred under nitrogen for 2 hours. The reaction mixture was slowly poured into an ice-cold citric acid (230 mL of 10% w/v, 119.7 mmol) aqueous solution under stirring. The resulting solid suspension was extracted with EtOAc (100 mL and 2×50 mL). After drying over sodium sulfate, evaporation of the solvents gave a residue that was dissolved in DCM and purified by flash chromatography on silica gel (120 g column) using a gradient of MeOH (0 to 5% over 30 min) in dichloromethane. The product eluted around 3-4% MeOH. Evaporation of the solvents and several cycle of trituration/evaporation in DCM/hexanes gave tert-butyl (16R,20R)-12-(2,6-dimethylphenyl)-20-(2-methylpropyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (499 mg, 45%) as a white solid. ¹H NMR (500 MHz, DMSO-d₆+10% D₂O) 2 rotamers visible (70:30) δ 8.34-8.11 (m, 1H), 7.84 (d, J=7.7 Hz, 1H), 7.64 (t, J=7.8 Hz, 1H), 7.52 (d, J 7.6 Hz, 1H), 7.25 (t, J=7.6 Hz, 1H), 7.11 (d, J=7.7 Hz, 2H), 6.17 (s, 1H), 5.70-5.46 (m, 1H), 5.08-4.90 (m, 1H), 4.22-4.05 (m, 1H), 4.03-3.86 (m, 1H), 3.49-3.31 (m, 1H), 3.20-2.91 (m, 3H), 2.02 (broad s, 6H), 1.62-1.51 (m, 1H), 1.46-1.36 (m, 11H), 0.97-0.85 (m, 6H). ESI-MS m/z calc. 635.2778, found 636.4 (M+1)⁺; Retention time: 1.89 minutes (LC method A).

### Example 49: Preparation of Compound 107

### Step 1: (3S)-4-Benzyl-6-hydroxy-3-isobutyl-1,4-diazepan-2-one

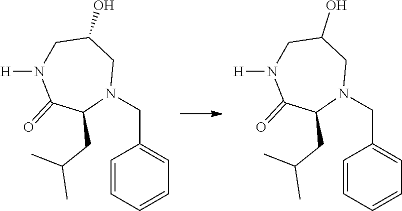 [SMILES: CC(C)C[C@H]1C(=O)NC[C@H](O)CN1Cc1ccccc1 ; CC(C)C[C@H]1C(=O)NCC(O)CN1Cc1ccccc1]

Into a solution of (3S,6S)-4-benzyl-6-hydroxy-3-isobutyl-1,4-diazepan-2-one (5.63 g, 20.371 mmol) in DCM (112 mL) was added Dess-Martin periodinane (12.960 g, 30.556 mmol) at 0° C. The reaction was stirred at room temperature for 2 hours. The reaction was quenched with a mixture of saturated 1:1 mixture of Na₂S₂O₃ and sodium bicarbonate (100 mL), and it was extracted with DCM (3×100 mL). The combined organic layers were washed with brine (100 mL), dried over anhydrous sodium sulfate and concentrated in vacuo. The residue was dissolved in methanol (281 mL). Sodium borohydride (2.3121 g, 2.4467 mL, 61.113 mmol) was added to the reaction mixture at room temperature and stirred for 1 hour. The reaction was quenched with water (100 mL). The product was extracted with ethyl acetate (3×200 mL). The combined organic layers were washed with brine (100 mL), dried over anhydrous sodium sulfate and concentrated under vacuum. The residue was purified by silica gel chromatography using 50 to 100% hexane-ethyl acetate to furnish (3S)-4-benzyl-6-hydroxy-3-isobutyl-1,4-diazepan-2-one (1.793 g, 32%) as a light yellow foamy solid. ¹H NMR (250 MHz, CDCl₃) δ 7.52-6.96 (m, 5H), 6.12 (s, 1H), 3.99-2.51 (m, 8H), 1.94-1.63 (m, 2H), 1.63-1.37 (m, 1H), 1.05-0.68 (m, 6H). The product is a mixture of diastereomers. ESI-MS m/z calc. 276.18378, found 276.9 (M+1)⁺; Retention time: 2.22 minutes; LC method S.

### Step 2: (2S)-1-Benzyl-2-isobutyl-1,4-diazepan-6-ol

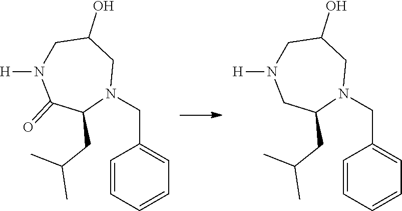 [SMILES: CC(C)C[C@H]1C(=O)NCC(O)CN1Cc1ccccc1 ; CC(C)C[C@H]1CNCC(O)CN1Cc1ccccc1]

Into a solution of (3S)-4-benzyl-6-hydroxy-3-isobutyl-1,4-diazepan-2-one (1.793 g, 6.4227 mmol) in anhydrous THF (40 mL) was added LAH (1.4626 g, 1.5950 mL, 38.536 mmol). The reaction mixture was stirred at 40° C. for 2 days. The reaction was quenched with water (1.5 mL), 15% NaOH (aqueous) (1.5 mL) and water (4.5 mL) subsequently. After stirring at room temperature for 1 hour, the white precipitate was removed by filtration through a pad of Celite. The filtrate was dried over anhydrous magnesium sulfate and concentrated in vacuo to furnish (2S)-1-benzyl-2-isobutyl-1,4-diazepan-6-ol (1.598 g, 95%) as a yellow oil. The product is a mixture of diastereomers. ESI-MS m/z calc. 262.2045, found 263.2 (M+1)⁺; Retention time: 1.96 minutes; LC method S.

### Step 3: tert-Butyl (3S)-4-benzyl-6-hydroxy-3-isobutyl-1,4-diazepane-1-carboxylate

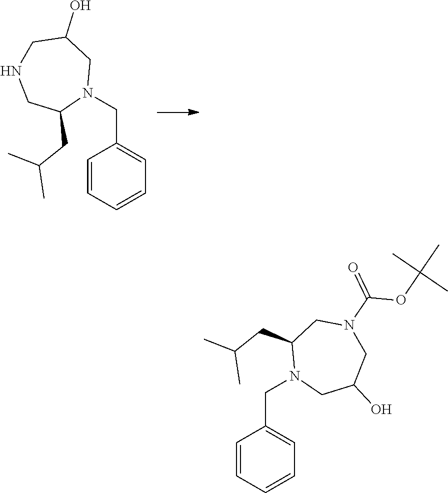 [SMILES: CC(C)C[C@H]1CNCC(O)CN1Cc1ccccc1 ; CC(C)C[C@H]1CN(C(=O)OC(C)(C)C)CC(O)CN1Cc1ccccc1]

Into a solution of (2S)-1-benzyl-2-isobutyl-1,4-diazepan-6-ol (1.598 g, 6.0902 mmol) in dichloromethane (20 mL), was added Boc anhydride (1.9938 g, 9.1353 mmol) and triethylamine (0.924 g, 9.1353 mmol). The reaction was stirred at room temperature for 2 hours. The reaction was quenched with brine (50 mL). The two layers were separated, and the aqueous layer was extracted with dichloromethane (2×50 mL). The combined organic layers were dried over anhydrous magnesium sulfate and concentrated under vacuum. The residue was purified by silica gel chromatography using 0 to 100% hexane-diethyl ether to furnish tert-butyl (3S)-4-benzyl-6-hydroxy-3-isobutyl-1,4-diazepane-1-carboxylate (1.359 g, 61%) as a clear gel. The compound is a mixture of diastereomers. ESI-MS m/z calc. 362.25696, found 363.3 (M+1)⁺; Retention time: 3.88 minutes; LC method S.

### Step 4: (2S)-1-Benzyl-2-isobutyl-1,4-diazepan-6-ol

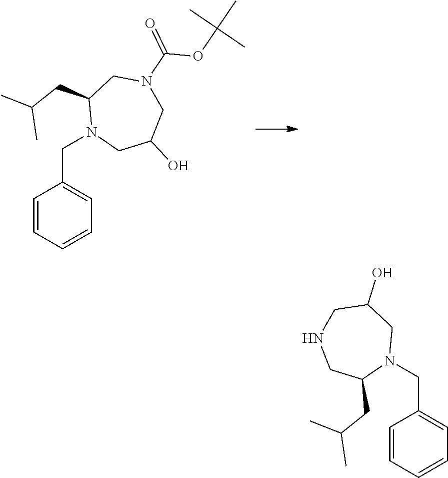 [SMILES: CC(C)C[C@H]1CN(C(=O)OC(C)(C)C)CC(O)CN1Cc1ccccc1 ; CC(C)C[C@H]1CNCC(O)CN1Cc1ccccc1]

Into a solution of tert-butyl (3S)-4-benzyl-6-hydroxy-3-isobutyl-1,4-diazepane-1-carboxylate (680 mg, 1.8758 mmol) in DCM (25 mL) was added HCl (10 mL of 4 M, 40.000 mmol) in dioxane. The reaction mixture was stirred at room temperature for 16 hours. The solvent was removed under reduced pressure. The residue was dissolved in water and lyophilized to furnish (2S)-1-benzyl-2-isobutyl-1,4-diazepan-6-ol (dihydrochloride salt) (628.4 mg, 96%) as a white solid. ¹H NMR (250 MHz, dimethylsuloxide-d₆) δ 11.93-9.24 (m, 2H), 7.99-7.05 (m, 5H), 4.85-3.60 (m, 7H), 3.51-2.80 (m, 5H), 2.04-1.20 (m, 3H), 1.15-0.42 (m, 6H). The product is a mixture of diastereomers. ESI-MS m/z calc. 262.2045, found 263.1 (M+1)⁺; Retention time: 1.27 minutes; LC method W.

### Step 5: 3-[(3S)-4-Benzyl-6-hydroxy-3-isobutyl-1,4-diazepane-1-carbonyl]-N-[4-chloro-6-(2,6-dimethylphenyl)pyrimidin-2-yl]benzenesulfonamide

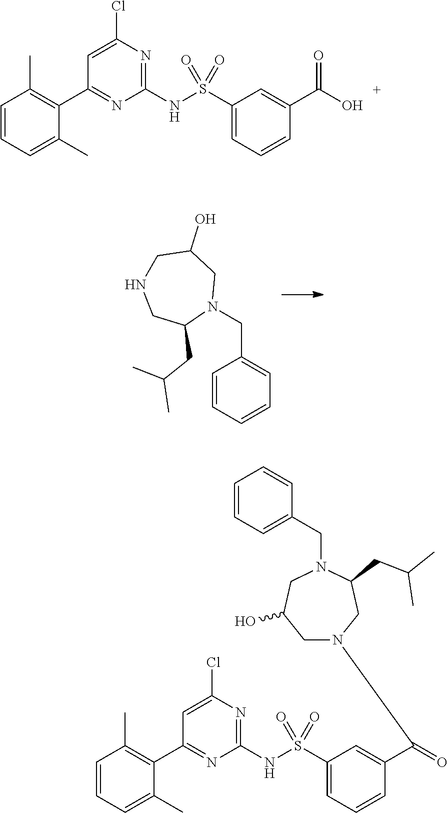 [SMILES: Cc1cccc(C)c1-c1cc(Cl)nc(NS(=O)(=O)c2cccc(C(=O)O)c2)n1 ; CC(C)C[C@H]1CNCC(O)CN1Cc1ccccc1 ; Cc1cccc(C)c1-c1cc(Cl)nc(NS(=O)(=O)c2cccc(C(=O)N3CC(O)CN(Cc4ccccc4)[C@@H](CC(C)C)C3)c2)n1]

A 100 mL flask was charged under nitrogen with (2S)-1-benzyl-2-isobutyl-1,4-diazepan-6-ol (hydrochloride salt) (602 mg, 2.014 mmol), anhydrous DMF (10 mL) and 3-[[4-chloro-6-(2,6-dimethylphenyl)pyrimidin-2-yl]sulfamoyl]benzoic acid (767 mg, 1.836 mmol). After dissolution of the reagents, the mixture was cooled down in an ice bath. DIEA (2.1 mL, 12.06 mmol) and HATU (847 mg, 2.228 mmol) were added and the mixture was stirred at 0° C. for 15 min. The reaction was quenched by being poured in citric acid (70 mL of 10% w/v, 36.43 mmol) (10% aqueous) under vigorous stirring and cooled in ice. The resulting white solid was filtered and washed with water. The wet solid was dissolved in DCM and the solution was dried over sodium sulfate. After evaporation of the solvents, the residue was purified by flash chromatography on silica gel (80 g column) using a gradient of methanol (0 to 5% over 30 min) in dichloromethane. The product eluted around 2-4% methanol. Evaporation of the solvents gave 3-[(3S)-4-benzyl-6-hydroxy-3-isobutyl-1,4-diazepane-1-carbonyl]-N-[4-chloro-6-(2,6-dimethylphenyl)pyrimidin-2-yl]benzenesulfonamide (493 mg, 41%) as a glassy resin. ¹H NMR (400 MHz, DMSO-d₆) δ 8.01 (broad s, 2H), 7.93 (s, 2H), 7.81-7.52 (m, 2H), 7.45-7.20 (m, 5H), 7.20-7.03 (m, 2H), 4.33-3.29 (m, 4H), 2.08-1.76 (m, 6H), 1.38-0.90 (m, 3H), 0.87-0.63 (m, 3H), 0.50 (d, J=6.2 Hz, 1.5H), 0.20 (d, J=6.3 Hz, 1.5H). ESI-MS m/z calc. 661.24896, found 662.35 (M+1)⁺; Retention time: 1.49 minutes; LC method A.

### Step 6: (19S)-18-Benzyl-12-(2,6-dimethylphenyl)-19-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 107)

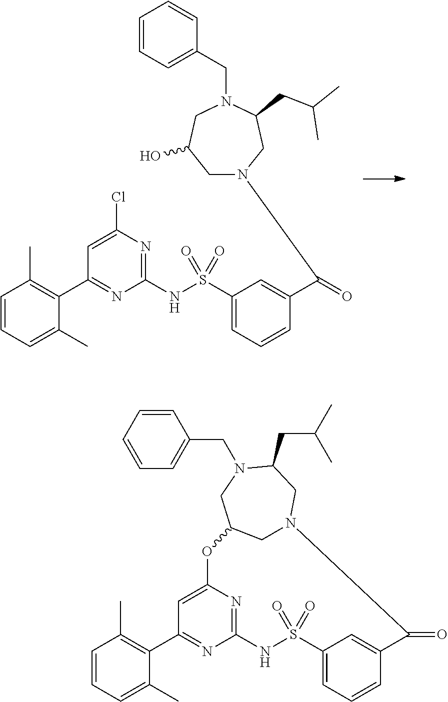 [SMILES: Cc1cccc(C)c1-c1cc(Cl)nc(NS(=O)(=O)c2cccc(C(=O)N3CC(O)CN(Cc4ccccc4)[C@@H](CC(C)C)C3)c2)n1 ; Cc1cccc(C)c1-c1cc2nc(n1)NS(=O)(=O)c1cccc(c1)C(=O)N1CC(CN(Cc3ccccc3)[C@@H](CC(C)C)C1)O2]

A 100 mL flask was charged under nitrogen with 3-[(3S)-4-benzyl-6-hydroxy-3-isobutyl-1,4-diazepane-1-carbonyl]-N-[4-chloro-6-(2,6-dimethylphenyl)pyrimidin-2-yl]benzenesulfonamide (482 mg, 0.7278 mmol) and anhydrous DMF (25 mL). The mixture was cooled down in ice. NaH (260 mg of 60% w/w, 6.501 mmol) (60% mineral oil dispersion) was added quickly. The mixture was stirred under nitrogen at 0° C. for 4 hours. The mixture was slowly poured into an ice-cold citric acid (100 mL of 10% w/v, 52.05 mmol) (aqueous 10% solution) under stirring. The resulting solid suspension was extracted with EtOAc (3×60 mL). After drying over sodium sulfate, evaporation of the solvents gave a residue that was dissolved in DCM containing a bit of methanol (a solid started to crash out with DCM only) and purified by flash chromatography on silica gel (40 g column) using a gradient of MeOH (0 to 10% over 30 min) in dichloromethane. Two products eluted around 4-5% with poor separation. The fractions enriched in the minor cyclized product (eluted as a shoulder after the major product) were evaporated to give 74 mg of mixture. it was dissolved in DMSO (2 mL), microfiltered and subjected to reverse phase preparative HPLC (Cis) using a gradient of acetonitrile in water (1 to 99% over 15 min) and HCl as a modifier (2×950 μL injections). The two products were separated and isolated. The cyclized product, more polar was isolated after evaporation as a white solid. The product was dissolved in DMSO (1 mL) and purified a second time using the same method. Evaporation gave pure (19S)-18-benzyl-12-(2,6-dimethylphenyl)-19-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (14 mg, 3%). ESI-MS m/z calc. 625.2723, found 626.35 (M+1)⁺; Retention time: 1.62 minutes (LC method A).

### Example 50: Preparation of Compound 108, Compound 109, and Compound 110

### Step 1: (2S,6S)-1-Benzyl-2-isobutyl-1,4-diazepan-6-ol

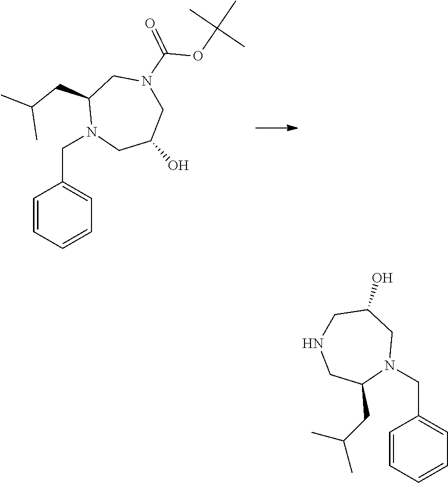 [SMILES: CC(C)C[C@H]1CN(C(=O)OC(C)(C)C)C[C@H](O)CN1Cc1ccccc1 ; CC(C)C[C@H]1CNC[C@H](O)CN1Cc1ccccc1]

Into a solution of tert-butyl (3S,6R)-4-benzyl-6-hydroxy-3-isobutyl-1,4-diazepane-1-carboxylate (1.727 g, 4.7641 mmol) in DCM (20 mL) was added HCl (10 mL of 4 M, 40.000 mmol) in dioxane. The reaction was stirred at room temperature for 16 hours. The solvent was removed under vacuum. The residue was dissolved in water and lyophilized to furnish (2S,6S)-1-benzyl-2-isobutyl-1,4-diazepan-6-ol (hydrochloride salt) (1.352 g, 90%) as an off-white solid. ¹H NMR (250 MHz, DMSO-d₆) δ 11.06 (m, 1H), 10.53-9.93 (m, 1H), 9.73 (s, 1H), 7.99-7.22 (m, 5H), 4.75-4.18 (m, 3H), 3.95-3.60 (m, 4H), 3.35-3.12 (m, 3H), 1.96-1.57 (m, 3H), 0.92 (m, 6H). ESI-MS m/z calc. 262.2045, found 263.4 (M+1)⁺; Retention time: 1.14 minutes; LC method W.

### Step 2: (2S,6S)-1-Benzyl-2-isobutyl-1,4-diazepan-6-ol

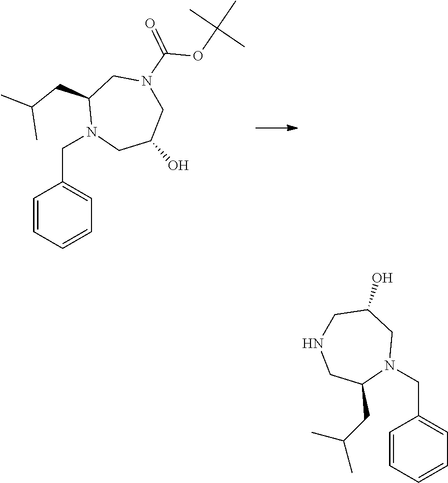 [SMILES: CC(C)C[C@H]1CN(C(=O)OC(C)(C)C)C[C@H](O)CN1Cc1ccccc1 ; CC(C)C[C@H]1CNC[C@H](O)CN1Cc1ccccc1]

Into a solution of tert-butyl (3S,6R)-4-benzyl-6-hydroxy-3-isobutyl-1,4-diazepane-1-carboxylate (1.727 g, 4.7641 mmol) in DCM (20 mL) was added HCl (10 mL of 4 M, 40.000 mmol) in dioxane. The reaction was stirred at room temperature for 16 hours. The solvent was removed under vacuum. The residue was dissolved in water and lyophilized to furnish (2S,6S)-1-benzyl-2-isobutyl-1,4-diazepan-6-ol (hydrochloride salt) (1.352 g, 90%) as an off-white solid. ¹H NMR (250 MHz, DMSO-d₆) δ 11.06 (m, 1H), 10.53-9.93 (m, 1H), 9.73 (s, 1H), 7.99-7.22 (m, 5H), 4.75-4.18 (m, 3H), 3.95-3.60 (m, 4H), 3.35-3.12 (m, 3H), 1.96-1.57 (m, 3H), 0.92 (m, 6H). ESI-MS m/z calc. 262.2045, found 263.4 (M+1)⁺; Retention time: 1.14 minutes; LC method W.

### Step 3: 3-[(3S,6R)-4-Benzyl-6-hydroxy-3-isobutyl-1,4-diazepane-1-carbonyl]-N-[4-chloro-6-(2,6-dimethylphenyl)pyrimidin-2-yl]benzenesulfonamide

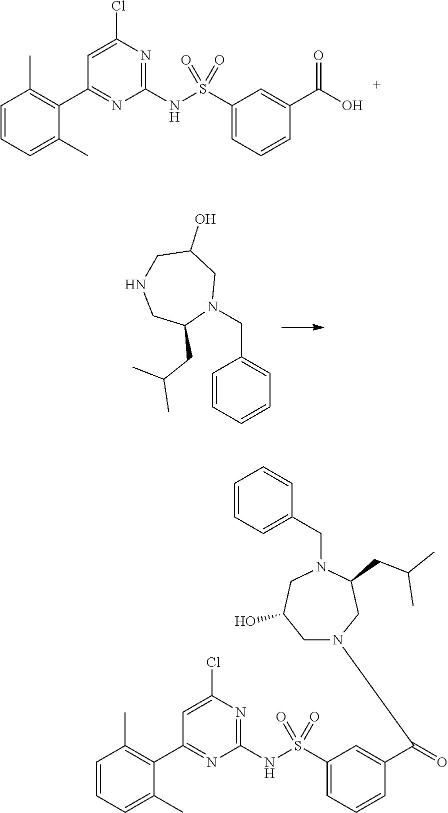 [SMILES: Cc1cccc(C)c1-c1cc(Cl)nc(NS(=O)(=O)c2cccc(C(=O)O)c2)n1 ; CC(C)C[C@H]1CNCC(O)CN1Cc1ccccc1 ; Cc1cccc(C)c1-c1cc(Cl)nc(NS(=O)(=O)c2cccc(C(=O)N3C[C@H](O)CN(Cc4ccccc4)[C@@H](CC(C)C)C3)c2)n1]

A 100 mL flask was charged under nitrogen with (2S)-1-benzyl-2-isobutyl-1,4-diazepan-6-ol (hydrochloride salt) (589 mg, 1.971 mmol), anhydrous DMF (10 mL) and 3-[[4-chloro-6-(2,6-dimethylphenyl)pyrimidin-2-yl]sulfamoyl]benzoic acid (683 mg, 1.634 mmol). After dissolution of the reagents, the mixture was cooled down in an ice bath. DIEA (1.9 mL, 10.91 mmol) and HATU (750 mg, 1.972 mmol) were added and the mixture was stirred at 0° C. for 13 min (LCMS showed complete reaction after 10 min). The reaction was quenched by being poured in citric acid (60 mL of 10% w/v, 31.23 mmol) (10% aqueous) under vigorous stirring and cooled in ice. The resulting white solid was filtered and washed with water. The wet solid was dissolved in DCM and the solution was dried over sodium sulfate. After evaporation of the solvents, the residue was purified by flash chromatography on silica gel (80 g column) using a gradient of methanol (0 to 5% over 30 min) in dichloromethane. The product eluted around 2-4% methanol. Evaporation of the solvents gave 3-[(3S,6R)-4-benzyl-6-hydroxy-3-isobutyl-1,4-diazepane-1-carbonyl]-N-[4-chloro-6-(2,6-dimethylphenyl)pyrimidin-2-yl]benzenesulfonamide (698 mg, 65%) as a white foamy solid. ESI-MS m/z calc. 661.24896, found 1.49 (M+1)⁺; Retention time: 662.46 minutes; LC method A.

### Step 4: (16R,19S)-18-Benzyl-12-(2,6-dimethylphenyl)-19-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 109)

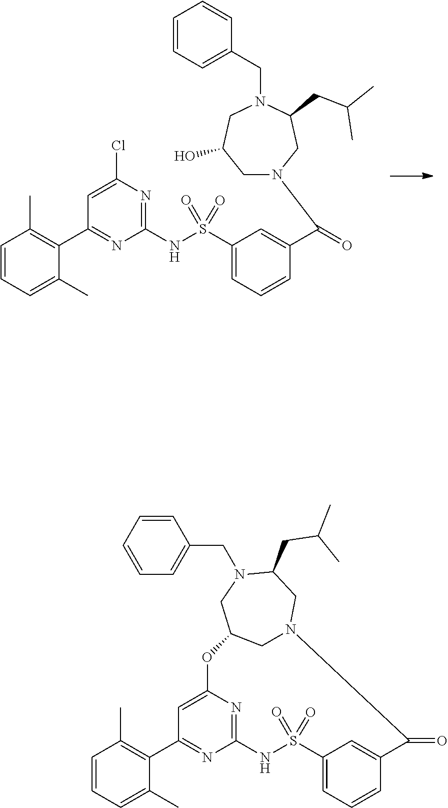 [SMILES: Cc1cccc(C)c1-c1cc(Cl)nc(NS(=O)(=O)c2cccc(C(=O)N3C[C@H](O)CN(Cc4ccccc4)[C@@H](CC(C)C)C3)c2)n1 ; Cc1cccc(C)c1-c1cc2nc(n1)NS(=O)(=O)c1cccc(c1)C(=O)N1C[C@@H](CN(Cc3ccccc3)[C@@H](CC(C)C)C1)O2]

A 100 mL flask was charged under nitrogen with 3-[(3S)-4-benzyl-6-hydroxy-3-isobutyl-1,4-diazepane-1-carbonyl]-N-[4-chloro-6-(2,6-dimethylphenyl)pyrimidin-2-yl]benzenesulfonamide (695 mg, 1.049 mmol) and anhydrous DMF (35 mL). The mixture was cooled down in ice. NaH (373 mg of 60% w/w, 9.326 mmol) (60% mineral oil dispersion) was added quickly. The mixture was stirred under nitrogen at 0° C. for 7 hours. The mixture was slowly poured into an ice-cold citric acid (150 mL of 10% w/v, 78.07 mmol) solution (10% aqueous) under stirring. The resulting solid suspension was extracted with EtOAc (3×60 mL). After drying over sodium sulfate, evaporation of the solvents gave a residue (1.9 g) that was dissolved in DCM containing a bit of methanol (a solid started to crash out with DCM only) and purified by flash chromatography on silica gel (40 g column) using a gradient of EtOAc (0 to 10% over 30 min. then 10-100% over 20 min) in dichloromethane. The product eluted around 10-35% EtOAc alongside dimeric impurities. The product was purified a second time using a 40 g silica column and a gradient of MeOH (0-10% over 30 min) in dichloromethane. The product eluted around 3-4% MeOH. Evaporation of the solvents and several cycle of trituration/evaporation in DCM/hexanes gave (16R,19S)-18-benzyl-12-(2,6-dimethylphenyl)-19-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (182 mg, 28%) as a white solid. ESI-MS m/z calc. 625.2723, found 626.39 (M+1)⁺; Retention time: 1.62 minutes (LC method A). This material was used for the next step without any further purification.

18 mg of material was dissolved in DMSO (1 μL) and purified by reverse phase preparative HPLC (Cis) using a gradient of acetonitrile in water (1 to 99% over 15 min) and HCl as a modifier to give (16R,19S)-18-benzyl-12-(2,6-dimethylphenyl)-19-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (12 mg, 65%) as an off-white solid. ¹H NMR (400 MHz, DMSO-d₆+10% D₂O) δ 8.53 (s, 1H), 7.98 (ddd, J=7.3, 5.4, 3.3 Hz, 1H), 7.79-7.69 (m, 2H), 7.63-7.38 (m, 5H), 7.31 (t, J=7.6 Hz, 1H), 7.15 (d, J=7.7 Hz, 2H), 6.23 (s, 1H), 5.74 (broad s, 1H), 4.60-4.20 broad s, 2H overlapped with water), 3.94-3.17 (br m, 7H), 2.03 (br s, 6H), 1.95-1.87 (m, 1H), 1.54 (br s, 2H), 0.94 (d, J=6.5 Hz, 3H), 0.90 (d, J=6.5 Hz, 3H). Exchangeable sulfonamide NH single visible at 10.10 ppm in the absence of D20. ESI-MS m/z calc. 625.2723, found 626.42 (M+1)⁺; Retention time: 1.62 minutes; LC method A.

### Step 5: (16R,19S)-12-(2,6-Dimethylphenyl)-19-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 108)

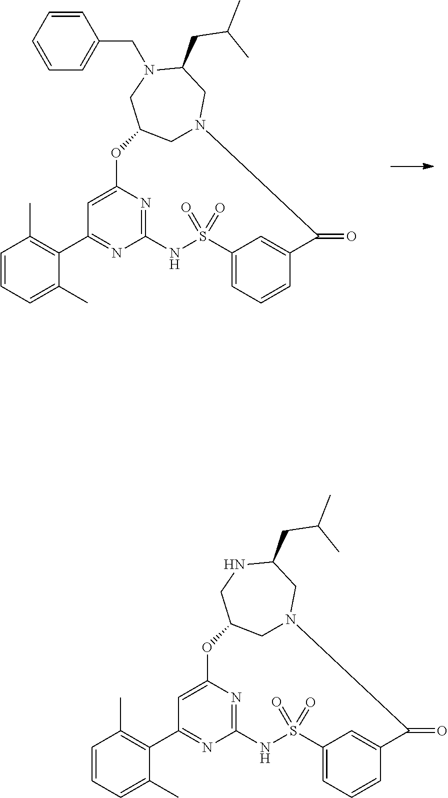 [SMILES: Cc1cccc(C)c1-c1cc2nc(n1)NS(=O)(=O)c1cccc(c1)C(=O)N1C[C@@H](CN(Cc3ccccc3)[C@@H](CC(C)C)C1)O2 ; Cc1cccc(C)c1-c1cc2nc(n1)NS(=O)(=O)c1cccc(c1)C(=O)N1C[C@H](CC(C)C)NC[C@H](C1)O2]

A 100 mL flask was charged with (19S)-18-benzyl-12-(2,6-dimethylphenyl)-19-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (151 mg, 0.2413 mmol) and MeOH (35 mL). The solution was sparged with nitrogen for 10 minutes. Pd(OH)₂ (76 mg of 20% w/w, 0.1082 mmol) was added and the solution was stirred under an atmosphere of hydrogen (balloon) for 21 hours. The solution was purged with nitrogen. The catalyst was removed by filtration through Celite and the filtrate was concentrated. The residue was dissolved in DCM and the solution was filtered. Evaporation of the solvent and trituration of the residue in DCM/hexanes followed by evaporation gave (16R,19S)-12-(2,6-dimethylphenyl)-19-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (137 mg, 102%) as an off-white solid. The material was used for the next step without any further purification. ESI-MS m/z calc. 535.22534, found 536.44 (M+1)⁺; Retention time: 0.96 minutes; LC method A.

### Step 6: (16R,19S)-18-(4,4-Difluorocyclohexyl)-12-(2,6-dimethylphenyl)-19-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 110)

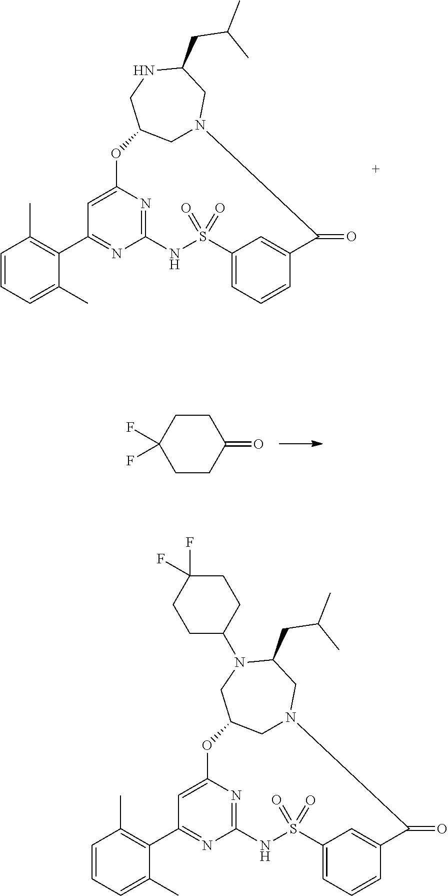 [SMILES: Cc1cccc(C)c1-c1cc2nc(n1)NS(=O)(=O)c1cccc(c1)C(=O)N1C[C@H](CC(C)C)NC[C@H](C1)O2 ; O=C1CCC(F)(F)CC1 ; Cc1cccc(C)c1-c1cc2nc(n1)NS(=O)(=O)c1cccc(c1)C(=O)N1C[C@@H](CN(C3CCC(F)(F)CC3)[C@@H](CC(C)C)C1)O2]

A 4 mL vial was charged with (19S)-12-(2,6-dimethylphenyl)-19-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (17 mg, 0.03059 mmol), anhydrous DCM (400 μL), 4,4-difluorocyclohexanone (35 mg, 0.2610 mmol) and acetic acid (12 μL, 0.2110 mmol). The vial was briefly purged with nitrogen, capped and stirred at room temperature for about 10 minutes. Sodium triacetoxyborohydride (22 mg, 0.1038 mmol) was added. The vial was purged with nitrogen, capped and the reaction was stirred at room temperature for 15 hours. A second number of reagents, namely 4,4-difluorocyclohexanone (115 mg, 0.8574 mmol) and sodium triacetoxyborohydride (42 mg, 0.1982 mmol) were added and the mixture was stirred at room temperature for 6 hours (30% conversion). Another load of the same reagents was added, and the mixture stirred at room temperature overnight. After 18 hours methanol (100 μL) was added and DCM was evaporated by blowing nitrogen in the vial. The residue was taken in DMSO (1 mL). The solution was microfiltered and purified by reverse phase preparative HPLC (Cis) using a gradient of acetonitrile in water (1 to 99% over 15 min) and HCl as a modifier to give a solid (85% pure) that was purified a second time. Evaporation of the solvents gave (16R,19S)-18-(4,4-difluorocyclohexyl)-12-(2,6-dimethylphenyl)-19-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (3.6 mg, 17%) as an off-white solid. ESI-MS m/z calc. 653.2847, found 654.4 (M+1)⁺; Retention time: 1.37 minutes; LC method A.

### Example 51: Preparation of Compound 111

### Step 1: (2R,6R)-1-Benzyl-2-isobutyl-1,4-diazepan-6-ol

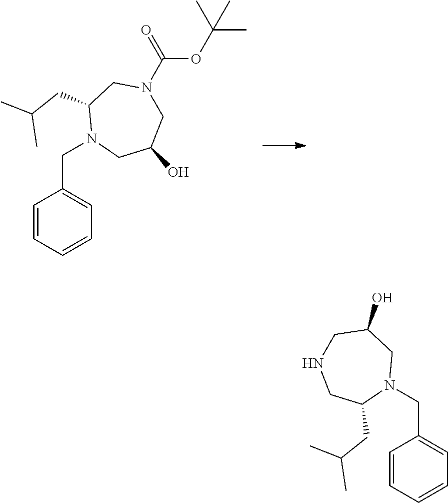 [SMILES: CC(C)C[C@@H]1CN(C(=O)OC(C)(C)C)C[C@@H](O)CN1Cc1ccccc1 ; CC(C)C[C@@H]1CNC[C@@H](O)CN1Cc1ccccc1]

To a solution of tert-butyl (3R,6S)-4-benzyl-6-hydroxy-3-isobutyl-1,4-diazepane-1-carboxylate (3.51 g, 9.6826 mmol) in DCM (40 mL) was added HCl (19.5 mL of 4 M, 78.000 mmol) in dioxane. The reaction was stirred at room temperature overnight. The reaction was concentrated in vacuum and the residue was dissolved in water and lyophilized to give (2R,6R)-1-benzyl-2-isobutyl-1,4-diazepan-6-ol (2.74 g, 102%). ¹H NMR (250 MHz, DMSO-d₆) δ 11.33-10.88 (m, 1H), 10.48-9.98 (m, 1H), 9.74 (s, 1H), 7.70 (s, 2H), 7.45 (s, 3H), 4.74-4.25 (m, 3H), 4.16-3.96 (m, 1H), 3.85-3.59 (m, 4H), 3.52-3.34 (m, 3H), 3.32-3.15 (m, 2H), 1.98-1.51 (m, 3H), 1.09-0.79 (m, 6H). ESI-MS m/z calc. 262.2045, found 263.3 (M+1)⁺; Retention time: 1.43 minutes; LC method T.

### Step 2: 3-[(3R,6S)-4-Benzyl-6-hydroxy-3-isobutyl-1,4-diazepane-1-carbonyl]-N-[4-chloro-6-(2,6-dimethylphenyl)pyrimidin-2-yl]benzenesulfonamide

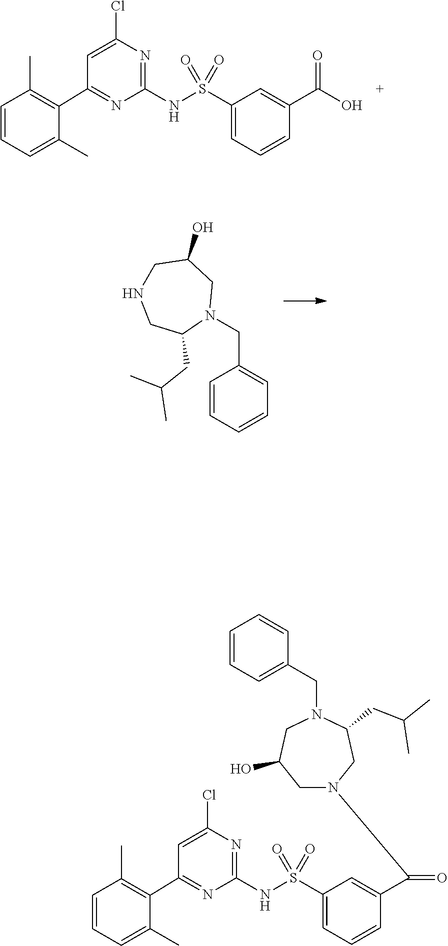 [SMILES: Cc1cccc(C)c1-c1cc(Cl)nc(NS(=O)(=O)c2cccc(C(=O)O)c2)n1 ; CC(C)C[C@@H]1CNC[C@@H](O)CN1Cc1ccccc1 ; Cc1cccc(C)c1-c1cc(Cl)nc(NS(=O)(=O)c2cccc(C(=O)N3C[C@@H](O)CN(Cc4ccccc4)[C@H](CC(C)C)C3)c2)n1]

A 100 mL flask was charged under nitrogen with (2R,6R)-1-benzyl-2-isobutyl-1,4-diazepan-6-ol (Dihydrochloride salt) (1.238 g, 3.692 mmol), anhydrous DMF (15 mL) and 3-[[4-chloro-6-(2,6-dimethylphenyl)pyrimidin-2-yl]sulfamoyl]benzoic acid (1.28 g, 3.063 mmol). After dissolution of the reagents, the mixture was cooled down in an ice bath. DIEA (3.5 mL, 20.09 mmol) and HATU (1.47 g, 3.866 mmol) were added and the mixture was stirred at 0° C. for 17 min. The reaction was quenched by being poured in citric acid (110 mL of 10% w/v, 57.25 mmol)(10% aqueous) under vigorous stirring and cooled in ice. The resulting white solid was filtered and washed with water. The wet solid was dissolved in DCM and the solution was dried over sodium sulfate. After evaporation of the solvents, the residue was dissolved in DCM and purified by flash chromatography on silica gel (120 g column) using a gradient of methanol (0 to 5% over 30 min) in dichloromethane. The product eluted around 2-3% methanol. Evaporation of the solvents gave 3-[(3R,6S)-4-benzyl-6-hydroxy-3-isobutyl-1,4-diazepane-1-carbonyl]-N-[4-chloro-6-(2,6-dimethylphenyl)pyrimidin-2-yl]benzenesulfonamide (970 mg, 48%) as a white foamy solid. ¹H NMR (500 MHz, DMSO-d₆) presence of several conformers, rendering annotation difficult and uncertain. Residual DMF also present. δ 8.06-7.97 (m, 2H), 7.83-7.72 (m, 1H), 7.70-7.58 (m, 1H), 7.41-7.17 (m, 7H), 7.16-7.05 (m, 2H), 4.96 (broad s, 0.4H), 4.16-3.92 (m, 1H), 3.89-3.56 (m, 1H), 3.37-3.12 (m, 6H overlap with water signal), 3.00-2.91 (m, 2H), 1.95-1.76 (m, 6H), 1.65-1.41 (m, 1H), 1.29-1.03 (m, 1H), 1.00-0.48 (m, 6H). ESI-MS m/z calc. 661.24896, found 662.36 (M+1)⁺; Retention time: 1.47 minutes; LC method A.

### Step 3: (16S,19R)-18-Benzyl-12-(2,6-dimethylphenyl)-19-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 111)

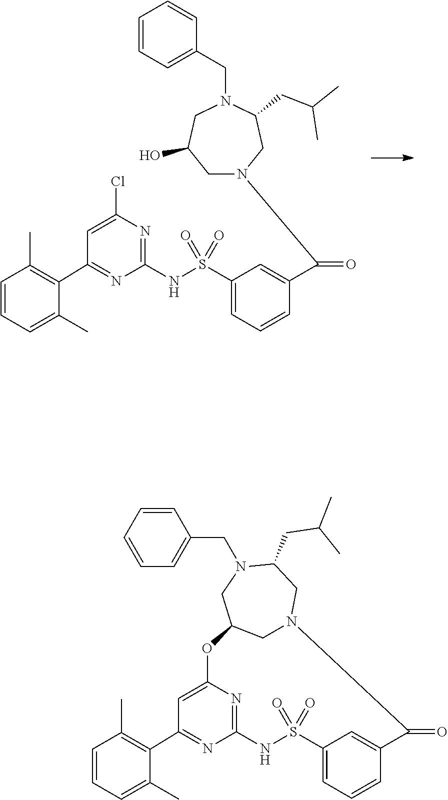 [SMILES: Cc1cccc(C)c1-c1cc(Cl)nc(NS(=O)(=O)c2cccc(C(=O)N3C[C@@H](O)CN(Cc4ccccc4)[C@H](CC(C)C)C3)c2)n1 ; Cc1cccc(C)c1-c1cc2nc(n1)NS(=O)(=O)c1cccc(c1)C(=O)N1C[C@H](CN(Cc3ccccc3)[C@H](CC(C)C)C1)O2]

A 100 mL flask was charged under nitrogen with 3-[(3R,6S)-4-benzyl-6-hydroxy-3-isobutyl-1,4-diazepane-1-carbonyl]-N-[4-chloro-6-(2,6-dimethylphenyl)pyrimidin-2-yl]benzenesulfonamide (947 mg, 1.430 mmol) and anhydrous DMF (50 mL). The mixture was cooled down in ice. NaH (513 mg of 60% w/w, 12.83 mmol) (60% mineral oil dispersion) was added quickly in 2 equal portions (additions separated by 2 min). The mixture was stirred under nitrogen at 0° C. for 5-10 minutes. The ice bath was removed, and the reaction was vigorously stirred under nitrogen for 4.5 hours. The mixture was slowly poured into an ice-cold citric acid (200 mL of 10% w/v, 104.1 mmol) solution (10% aqueous) under stirring. The resulting solid suspension was extracted with EtOAc (3×70 mL). After drying over sodium sulfate, evaporation of the solvents gave a residue that was dissolved in DCM containing a bit of methanol (a solid started to crash out with DCM only) and purified by flash chromatography on silica gel (80 g column) using a gradient of MeOH (0-10% over 30 min) in dichloromethane. The product eluted around 4-5% MeOH. Evaporation of the solvents gave 749 mg of 80% pure material. It was purified a second time using a shallower gradient (0-10% over 45 min). Evaporation of the solvents and several cycles of trituration evaporation in DCM/hexanes gave (16S,19R)-18-benzyl-12-(2,6-dimethylphenyl)-19-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (526 mg, 57%) as a white solid. ¹H NMR (500 MHz, DMSO-d₆) generally broad signals δ 12.96 (broad s, 1H), 8.48 (s, 1H), 7.94 (s, 1H), 7.81-7.57 (br m, 2H), 7.51-7.32 (m, 4H), 7.31-7.18 (m, 2H), 7.11 (d, J=7.2 Hz, 2H), 6.18 (br s, 1H), 5.69 (br s, 1H), 4.10-3.94 (m, 2H), 3.70 (d, J=14.5, 5.4 Hz, 1H), 3.61-3.44 (m, 3H), 3.25-3.12 (m, 2H), 2.99-2.92 (m, 1H), 1.98 (br s, 6H), 1.65-1.54 (m, 1H), 1.29-1.08 (m, 2H), 1.07-0.88 (m, 6H). ESI-MS m/z calc. 625.2723, found 626.37 (M+1)⁺; Retention time: 1.61 minutes; LC method A.

### Example 52: Preparation of Compound 112

### Step 1: tert-Butyl N-[(1R)-3-chloro-1-methyl-2-oxo-propyl]carbamate

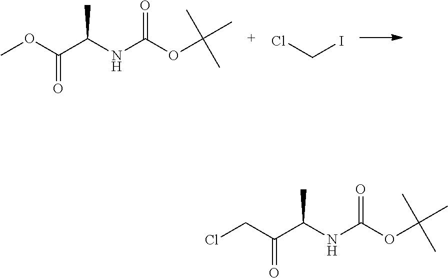

To a stirring solution of diisopropylamine (31.068 g, 43.030 mL, 307.03 mmol) in anhydrous THE (180 mL) at −78° C. under nitrogen was dropwise added n-BuLi (112.58 mL of 2.5 M, 281.45 mmol). After the addition was complete, the reaction was stirred at this temperature for 45 minutes. The prepared LDA solution was added via cannula to a cold (−78° C.) solution of methyl (2R)-2-(tert-butoxycarbonylamino)propanoate (10.4 g, 51.172 mmol) and chloro(iodo)methane (36.104 g, 204.69 mmol) in anhydrous THE (360 mL). After the LDA addition was complete, the resulting reaction mixture was stirred at −78° C. for 1 hour. The reaction was quenched cold by a dropwise addition of a solution of glacial acetic acid (50 mL) in THE (50 mL). The reaction mixture was warmed up to −0° C. and water (300 mL) was added. Volatiles were removed under vacuum and the aqueous layer was extracted with ethyl acetate (3×200 mL). The combined organic layers were washed with brine (70 mL), dried over anhydrous sodium sulfate and concentrated. The crude was purified by silica gel chromatography using 0-20% hexanes-ethyl acetate to afford pale-yellow solid of tert-butyl N-[(1R)-3-chloro-1-methyl-2-oxo-propyl]carbamate (10.84 g, 86%). ¹H NMR (250 MHz, CDCl₃) δ 5.09 (s, 1H), 4.62-4.43 (m, 1H), 4.28 (s, 2H), 1.44 (s, 9H), 1.37 (d, J=7.2 Hz, 3H).

### Step 2: Ethyl 2-[benzyl-[(3R)-3-(tert-butoxycarbonylamino)-2-oxo-butyl]amino]acetate

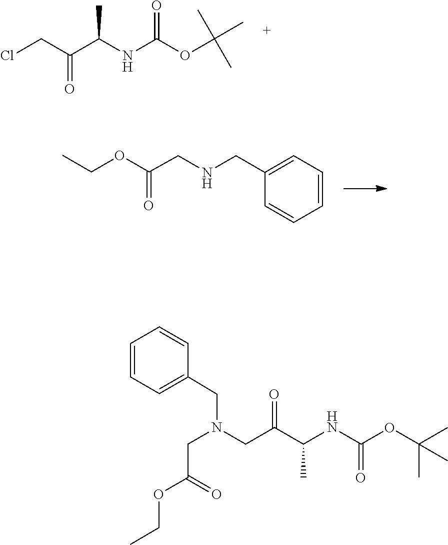 [SMILES: C[C@@H](NC(=O)OC(C)(C)C)C(=O)CCl ; CCOC(=O)CNCc1ccccc1 ; CCOC(=O)CN(CC(=O)[C@@H](C)NC(=O)OC(C)(C)C)Cc1ccccc1]

To a stirring solution of tert-butyl N-[(1R)-3-chloro-1-methyl-2-oxo-propyl]carbamate (10.84 g, 44.009 mmol) and ethyl 2-(benzylamino)acetate (hydrochloride salt) (10.109 g, 44.009 mmol) in anhydrous DMF (65 mL) at room temperature under nitrogen was added sodium bicarbonate (14.789 g, 176.04 mmol), followed by sodium iodide (4.6176 g, 30.806 mmol). The reaction mixture was stirred for 18 hours. Water (250 mL) was added and the product was extracted with ethyl acetate (3×150 mL). The combined organic layers were washed with brine (120 mL), dried over anhydrous sodium sulfate and concentrated. The crude was purified by silica gel chromatography using 0-25% hexanes-ethyl acetate to afford as a yellow oil ethyl 2-[benzyl-[(3R)-3-(tert-butoxycarbonylamino)-2-oxo-butyl]amino]acetate (13.56 g, 77%) ESI-MS m/z calc. 378.21548, found 379.6 (M+1)⁺; Retention time: 4.42 minutes; LC method S.

### Step 3: Ethyl 2-[benzyl-[(3R)-3-(tert-butoxycarbonylamino)-2-hydroxy-butyl]amino]acetate, diastereomer 1, and ethyl 2-[benzyl-[(3R)-3-(tert-butoxycarbonylamino)-2-hydroxy-butyl]amino]acetate, diastereomer 2

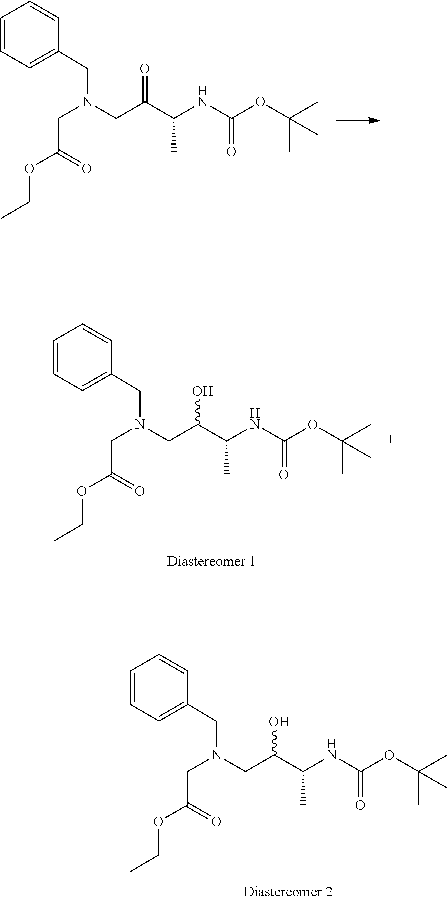

To a stirring solution of ethyl 2-[benzyl-[(3R)-3-(tert-butoxycarbonylamino)-2-oxo-butyl]amino]acetate (9.19 g, 24.282 mmol) in anhydrous methanol (110 mL) at −78° C. under nitrogen was added portionwise sodium borohydride (1.8373 g, 48.564 mmol). After the addition was complete, the reaction mixture was stirred at this temperature for 2 hours. The reaction was quenched at −78° C. with saturated aqueous ammonium chloride (250 mL) and then allowed to warm up to room temperature. Brine (100 mL) was added and the product was extracted with ethyl acetate (3×200 mL). The combined organic layers were dried over anhydrous sodium sulfate and concentrated. The crude product was purified by silica gel chromatography using 0-25% hexanes-ethyl acetate to afford two fractions: 1) diastereomer 1, minor isomer containing impurities (yellow oil), ethyl 2-[benzyl-[(3R)-3-(tert-butoxycarbonylamino)-2-hydroxy-butyl]amino]acetate (1.59 g, 16%) ESI-MS m/z calc. 380.2311, found 381.6 (M+1)⁺; Retention time: 3.96 minutes and 2) diastereomer 2, major isomer containing a significant portion of diastereomer 1 (yellow oil), ethyl 2-[benzyl-[(3R)-3-(tert-butoxycarbonylamino)-2-hydroxy-butyl]amino]acetate (8.78 g, 90%) ESI-MS m/z calc. 380.2311, found 381.6 (M+1)⁺; Retention time: 3.78 minutes, (LC method S).

### Step 4: (7R)-4-Benzyl-6-hydroxy-7-methyl-1,4-diazepan-2-one

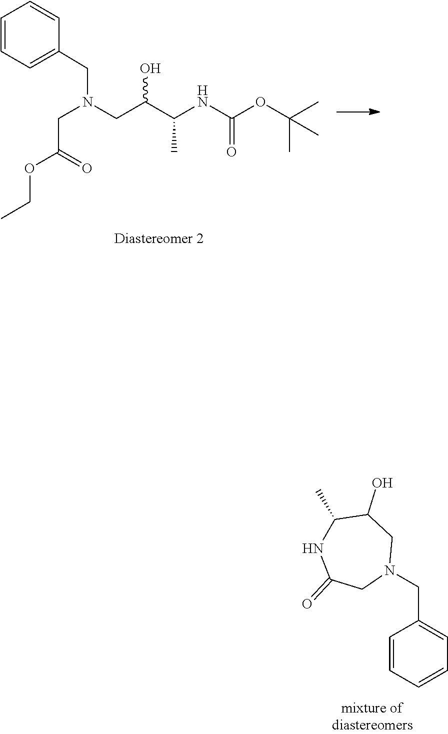 [SMILES: CCOC(=O)CN(Cc1ccccc1)CC(O)[C@@H](C)NC(=O)OC(C)(C)C ; C[C@H]1NC(=O)CN(Cc2ccccc2)CC1O]

Ethyl 2-[benzyl-[(3R)-3-(tert-butoxycarbonylamino)-2-hydroxy-butyl]amino]acetate (8.78 g, 23.076 mmol) was dissolved in a solution of HCl (144.22 mL of 4 M, 576.90 mmol) in 1,4-dioxane at room temperature and the reaction mixture was stirred for 1 hour. Volatiles were removed under vacuum and the obtained residue was dissolved in anhydrous ethanol (300 mL). TEA (23.351 g, 32.164 mL, 230.76 mmol) was added at room temperature and the reaction mixture was heated to 50° C. for 1 hour. After cooling to room temperature, the volatiles were removed under vacuum. Saturated aqueous sodium bicarbonate (250 mL) and brine (100 mL) were added and the product was extracted with ethyl acetate (3×200 mL). The combined organic layers were dried over anhydrous sodium sulfate and concentrated to afford as a yellow oil (7R)-4-benzyl-6-hydroxy-7-methyl-1,4-diazepan-2-one (5.59 g, 98%). ESI-MS m/z calc. 234.13683, found 235.4 (M+1)⁺; Retention time: 1.63 minutes; LC method S.

### Step 5: tert-Butyl (7R)-4-benzyl-6-hydroxy-7-methyl-1,4-diazepane-1-carboxylate, diastereomer 1, and tert-butyl (7R)-4-benzyl-6-hydroxy-7-methyl-1,4-diazepane-1-carboxylate, diastereomer 2

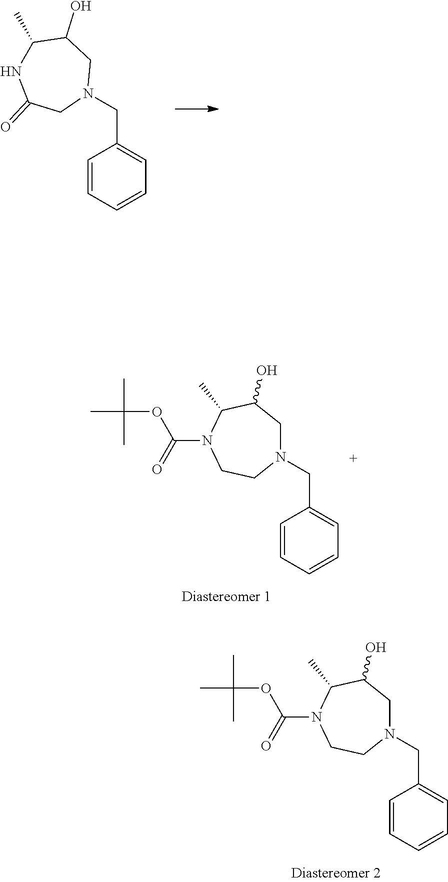 [SMILES: C[C@H]1NC(=O)CN(Cc2ccccc2)CC1O ; C[C@@H]1C(O)CN(Cc2ccccc2)CCN1C(=O)OC(C)(C)C ; C[C@@H]1C(O)CN(Cc2ccccc2)CCN1C(=O)OC(C)(C)C]

To a stirring solution of (7R)-4-benzyl-6-hydroxy-7-methyl-1,4-diazepan-2-one (5.59 g, 23.859 mmol) in anhydrous THF (220 mL) at 0° C. under nitrogen was added portionwise LAH (5.4332 g, 143.15 mmol). After the addition was complete, the reaction mixture was stirred at 0° C. for 10 minutes, then heated to 40° C. for 2 hours. The reaction mixture was cooled to 0° C. and quenched following a Fieser workup procedure. Salts were filtered off through a pad of Celite and washed with THE (2×100 mL). The combined filtrate was concentrated under vacuum. The residue was dissolved in a mixture of 1,4-dioxane (100 mL) and saturated aqueous sodium bicarbonate (100 mL), and Boc anhydride (5.7279 g, 26.245 mmol) was added. The reaction mixture was stirred for 2 hours at room temperature. Brine (200 mL) was added and the product was extracted with ethyl acetate (3×200 mL). The combined organic layers were dried over anhydrous sodium sulfate and concentrated. The crude was purified by silica gel chromatography using 0-60% hexanes-ethyl acetate to afford 2 distinct fractions: 1) less polar compound A (colorless oil), diastereomer 1, tert-butyl (7R)-4-benzyl-6-hydroxy-7-methyl-1,4-diazepane-1-carboxylate (840 mg, 10%) ESI-MS m/z calc. 320.21, found 321.4 (M+1)⁺; Retention time: 3.32 minutes (LC method S). ¹H NMR (250 MHz, CDCl₃) δ 7.47-7.17 (m, 5H), 3.96-3.75 (m, 2H), 3.72-3.57 (m, 2H), 3.21-2.92 (m, 2H), 2.89-2.70 (m, 1H), 2.54-2.17 (m, 2H), 1.45 (s, 9H), 1.30-1.09 (m, 4H); 2) more polar compound B (colorless oil), diastereomer 2, tert-butyl (7R)-4-benzyl-6-hydroxy-7-methyl-1,4-diazepane-1-carboxylate (3.25 g, 40%) ESI-MS m/z calc. 320.21, found 321.7 (M+1)⁺; Retention time: 3.26 minutes (LC method S). ¹H NMR (250 MHz, CDCl₃) δ 7.44-7.14 (m, 5H), 3.70-3.52 (m, 4H), 3.03-2.78 (m, 2H), 2.68-2.52 (m, 3H), 1.45 (s, 9H), 1.27 (d, J=1.9 Hz, 2H), 1.19 (d, J=6.8 Hz, 3H).

### Step 6: tert-Butyl (7R)-6-hydroxy-7-methyl-1,4-diazepane-1-carboxylate, diastereomer 1

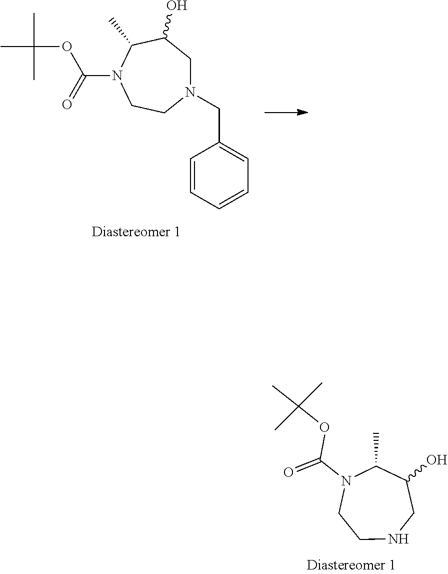 [SMILES: C[C@@H]1C(O)CN(Cc2ccccc2)CCN1C(=O)OC(C)(C)C ; C[C@@H]1C(O)CNCCN1C(=O)OC(C)(C)C]

To a stirring solution of tert-butyl (7R)-4-benzyl-6-hydroxy-7-methyl-1,4-diazepane-1-carboxylate (800 mg, 2.4967 mmol) in anhydrous methanol (23 mL) in a pressure vessel at room temperature was added palladium on carbon (398.54 mg, 10% w/w, 0.3745 mmol), followed by ammonium formate (629.73 mg, 9.9868 mmol). The reaction mixture was heated to 65° C. for 1 hour. After cooling to room temperature, the reaction mixture was filtered through a pad of Celite and filter cake was washed with methanol (2×20 mL). The combined filtrate was concentrated under vacuum to afford as a colorless oil tert-butyl (7R)-6-hydroxy-7-methyl-1,4-diazepane-1-carboxylate (670 mg, 99%) ESI-MS m/z calc. 230.16304, found 231.5 (M+1)⁺; Retention time: 2.39 minutes; LC method S.

### Step 7: 01-Benzyl 04-tert-butyl (5R)-6-hydroxy-5-methyl-1,4-diazepane-1,4-dicarboxylate, diastereomer 1

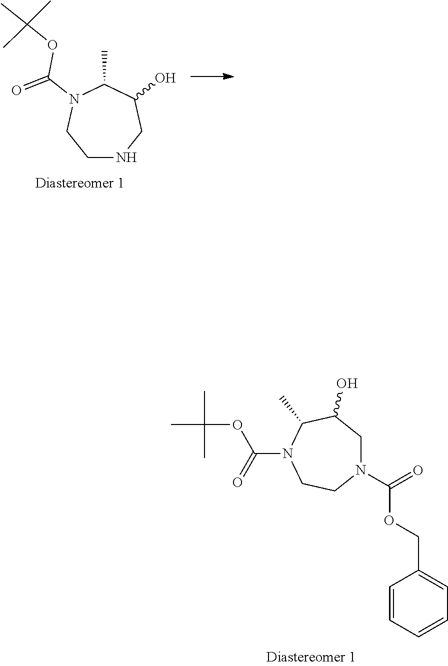 [SMILES: C[C@@H]1C(O)CNCCN1C(=O)OC(C)(C)C ; C[C@@H]1C(O)CN(C(=O)OCc2ccccc2)CCN1C(=O)OC(C)(C)C]

To a stirring solution of tert-butyl (7R)-6-hydroxy-7-methyl-1,4-diazepane-1-carboxylate (670 mg, 2.6183 mmol) in a mixture of 1,4-dioxane (15 mL) and saturated aqueous sodium bicarbonate (15 mL) at 0° C. was dropwise added benzyl chloroformate (705.25 mg, 0.5902 mL, 3.9274 mmol). After the addition was complete, the reaction mixture was stirred at 0° C. for 1 hour. The reaction was quenched cold with brine (25 mL) and the product was extracted with ethyl acetate (3×50 mL). The combined organic layers were washed with saturated aqueous sodium bicarbonate (50 mL) and brine (40 mL), dried over anhydrous sodium sulfate and concentrated. The crude was purified by silica gel chromatography using 0-40% hexanes-ethyl acetate to afford as a colorless oil 01-benzyl 04-tert-butyl (5R)-6-hydroxy-5-methyl-1,4-diazepane-1,4-dicarboxylate (354 mg, 36%). ¹H NMR (250 MHz, DMSO-d₆) δ 7.35 (d, J=3.2 Hz, 5H), 5.22-4.80 (m, 3H), 4.36-4.09 (m, 1H), 3.84-3.65 (m, 2H), 3.63-3.46 (m, 3H), 3.21-3.13 (m, 2H), 1.39-1.28 (m, 9H), 1.07 (d, J=6.8 Hz, 3H). ESI-MS m/z calc. 364.19983, found 365.1 (M+1)⁺; Retention time: 2.31 minutes; LC method T.

### Step 8: Benzyl (5R)-6-hydroxy-5-methyl-1,4-diazepane-1-carboxylate, diastereomer 1

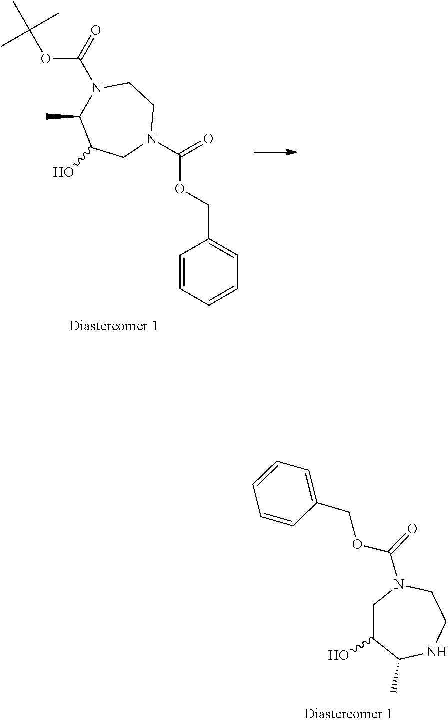 [SMILES: C[C@@H]1C(O)CN(C(=O)OCc2ccccc2)CCN1C(=O)OC(C)(C)C ; C[C@H]1NCCN(C(=O)OCc2ccccc2)CC1O]

A 100 mL round bottom flask was charged with 01-benzyl 04-tert-butyl (5R)-6-hydroxy-5-methyl-1,4-diazepane-1,4-dicarboxylate (339 mg, 0.9302 mmol), DCM (3.5 mL) and HCl (3 mL of 4 M, 12.00 mmol) (4M dioxane solution). The mixture was stirred at rt for 2.5 hours. The volatiles were removed under reduced pressure. The solid was treated with DCM and hexanes and the solvents were evaporated. The operation was repeated 3 times. Drying in vacuo provided benzyl (5R)-6-hydroxy-5-methyl-1,4-diazepane-1-carboxylate (hydrochloride salt) (291 mg, 98%) as a white foamy solid. ESI-MS m/z calc. 264.1474, found 265.1 (M+1)⁺; Retention time: 0.62 minutes; LC method A.

### Step 9: Benzyl (5R)-4-[3-[[4-chloro-6-(2,6-dimethylphenyl)pyrimidin-2-yl]sulfamoyl]benzoyl]-6-hydroxy-5-methyl-1,4-diazepane-1-carboxylate, diastereomer 1

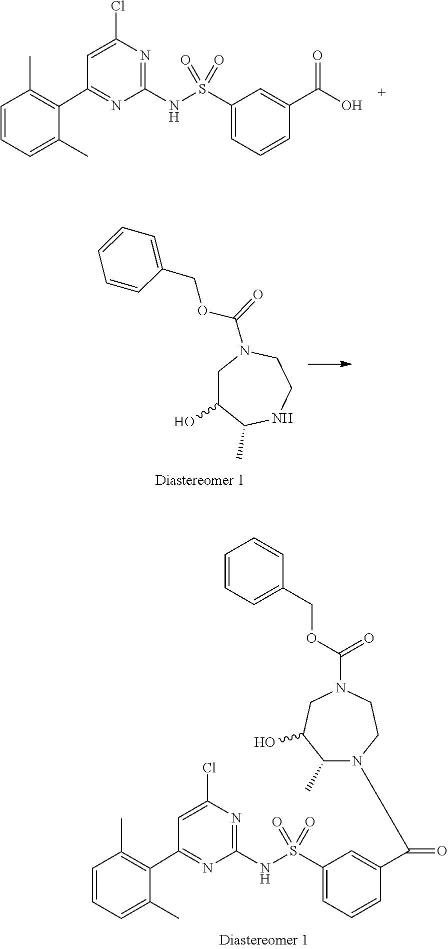 [SMILES: Cc1cccc(C)c1-c1cc(Cl)nc(NS(=O)(=O)c2cccc(C(=O)O)c2)n1 ; C[C@H]1NCCN(C(=O)OCc2ccccc2)CC1O ; Cc1cccc(C)c1-c1cc(Cl)nc(NS(=O)(=O)c2cccc(C(=O)N3CCN(C(=O)OCc4ccccc4)CC(O)[C@H]3C)c2)n1]

A 100 mL flask was charged under nitrogen with benzyl (5R)-6-hydroxy-5-methyl-1,4-diazepane-1-carboxylate (hydrochloride salt) (291 mg, 0.9385 mmol), anhydrous DMF (6 mL) and 3-[[4-chloro-6-(2,6-dimethylphenyl)pyrimidin-2-yl]sulfamoyl]benzoic acid (340 mg, 0.8137 mmol). After dissolution of the reagents, the mixture was cooled down in an ice bath. DIEA (0.95 mL, 5.454 mmol) and HATU (390 mg, 1.026 mmol) were added and the mixture was stirred at 0° C. for 3.5 hours. The reaction was quenched by being poured in citric acid (30 mL of 10% w/v, 15.61 mmol)(10% aqueous) under vigorous stirring and cooled in ice. The resulting white solid was filtered. The wet solid was dissolved in DCM and the solution was dried over sodium sulfate. After concentration, it was purified by flash chromatography on silica gel (40 g column) using a gradient of methanol (0 to 10% over 60 min) in dichloromethane. The product eluted around 2-3% methanol. Evaporation of the solvents gave benzyl (5R)-4-[3-[[4-chloro-6-(2,6-dimethylphenyl)pyrimidin-2-yl]sulfamoyl]benzoyl]-6-hydroxy-5-methyl-1,4-diazepane-1-carboxylate (340 mg, 63%) as a white foamy solid. ESI-MS m/z calc. 663.19183, found 664.38 (M+1)⁺; Retention time: 1.76 minutes (LC method J).

### Step 10: Benzyl (21R)-12-(2,6-dimethylphenyl)-21-methyl-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate, diastereomer 1 (Compound 112)

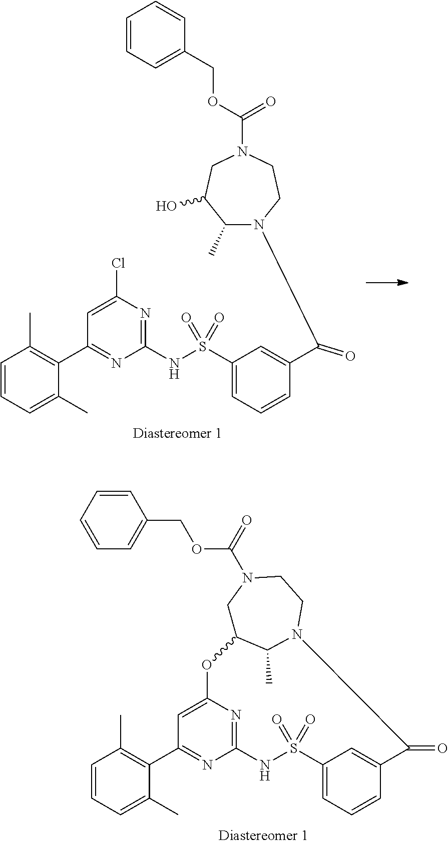 [SMILES: Cc1cccc(C)c1-c1cc(Cl)nc(NS(=O)(=O)c2cccc(C(=O)N3CCN(C(=O)OCc4ccccc4)CC(O)[C@H]3C)c2)n1 ; Cc1cccc(C)c1-c1cc2nc(n1)NS(=O)(=O)c1cccc(c1)C(=O)N1CCN(C(=O)OCc3ccccc3)CC(O2)[C@H]1C]

A 100 mL flask was charged under nitrogen with benzyl (5R)-4-[3-[[4-chloro-6-(2,6-dimethylphenyl)pyrimidin-2-yl]sulfamoyl]benzoyl]-6-hydroxy-5-methyl-1,4-diazepane-1-carboxylate (332 mg, 0.4999 mmol) and anhydrous DMF (17 mL). The mixture was cooled down in ice. NaH (183 mg of 60% w/w, 4.575 mmol) (60% mineral oil dispersion) was added. The mixture was stirred under nitrogen at 0° C. for 1 h 15 min. The reaction mixture was slowly poured into an ice-cold citric acid (70 mL of 10% w/v, 36.43 mmol) aqueous solution under stirring. The resulting solid suspension was extracted with EtOAc (3×50 mL). After drying over sodium sulfate, evaporation of the solvents gave a residue that was dissolved in DCM and purified by flash chromatography on silica gel (24 g column) using a gradient of MeOH (0 to 5% over 30 min) in dichloromethane. The product eluted around 2-3% MeOH. Evaporation of the solvents and several cycle of trituration/evaporation in DCM/hexanes gave benzyl (21R)-12-(2,6-dimethylphenyl)-21-methyl-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (56 mg, 17%) as a white solid. ¹H NMR (499 MHz, DMSO-d₆) presence of rotamers (1:1 ratio) δ 13.08 (very broad split s, 0.3H), 8.34 and 8.30 (two singlets 1:1, total 1H), 7.98-7.86 (broad m, 1H), 7.67 (br s, 2H), 7.46-7.38 (m, 1H), 7.35 (d, J=7.0 Hz, 1H), 7.31 (t, J=7.4 Hz, 1H), 7.29-7.18 (m, 3H), 7.12 (d, J=7.6 Hz, 2H), 6.44 (br s, 1H), 5.54-5.06 (m, 3H), 4.46-4.30 (m, 1H), 4.27-4.12 (m, 1H), 4.11-3.87 (m, 2H), 3.87-3.73 (m, 1H), 3.43-3.19 (m, 2H overlapped with water signal), 2.05 (br s, 6H), 1.09 (d, 1.5H), 1.08 (d, 1.5H). ESI-MS m/z calc. 627.21515, found 628.37 (M+1)⁺; Retention time: 1.61 minutes; LC method A.

### Example 53: Preparation of Compound 113

### Step 1: tert-Butyl (7R)-6-hydroxy-7-methyl-1,4-diazepane-1-carboxylate, diastereomer 2

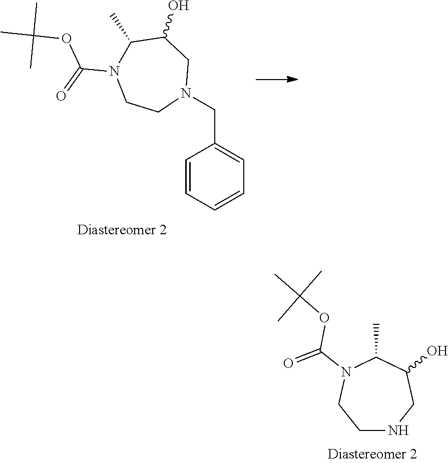 [SMILES: C[C@@H]1C(O)CN(Cc2ccccc2)CCN1C(=O)OC(C)(C)C ; C[C@@H]1C(O)CNCCN1C(=O)OC(C)(C)C]

To a stirring solution of tert-butyl (7R)-4-benzyl-6-hydroxy-7-methyl-1,4-diazepane-1-carboxylate (3.18 g, 9.9243 mmol) in anhydrous methanol (90 mL) in a pressure vessel at room temperature was added palladium on carbon (1.5842 g, 10% w/w, 1.4886 mmol), followed by ammonium formate (2.5031 g, 39.697 mmol). The reaction mixture was heated to 65° C. for 1 hour. After cooling to room temperature, the reaction mixture was filtered through a pad of Celite and filter cake was washed with methanol (2×50 mL). The combined filtrate was concentrated under vacuum to afford as a colorless oil tert-butyl (7R)-6-hydroxy-7-methyl-1,4-diazepane-1-carboxylate (2.201 g, 82%). ESI-MS m/z calc. 230.16304, found 231.7 (M+1)⁺; Retention time: 2.27 minutes; LC method S.

### Step 2: 01-Benzyl 04-tert-butyl (5R)-6-hydroxy-5-methyl-1,4-diazepane-1,4-dicarboxylate, diastereomer 2

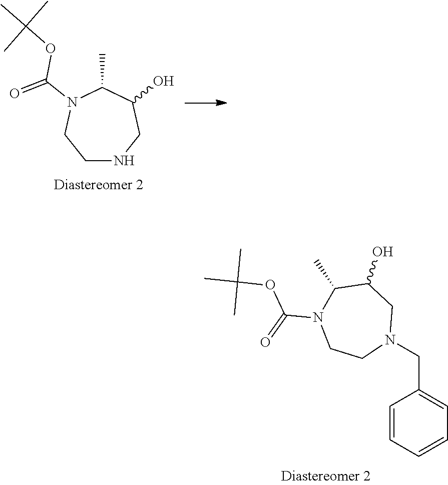 [SMILES: C[C@@H]1C(O)CNCCN1C(=O)OC(C)(C)C ; C[C@@H]1C(O)CN(Cc2ccccc2)CCN1C(=O)OC(C)(C)C]

To a stirring solution of tert-butyl (7R)-6-hydroxy-7-methyl-1,4-diazepane-1-carboxylate (2.201 g, 9.5569 mmol) in a mixture of 1,4-dioxane (50 mL) and saturated aqueous sodium bicarbonate (50 mL) at 0° C. was dropwise added benzyl chloroformate (2.4455 g, 2.0464 mL, 14.335 mmol). After the addition was complete, the reaction mixture was stirred at 0° C. for 1 hour. The reaction was quenched cold with brine (75 mL) and the product was extracted with ethyl acetate (3×120 mL). The combined organic layers were washed with saturated aqueous sodium bicarbonate (80 mL) and brine (40 mL), dried over anhydrous sodium sulfate and concentrated. The crude was purified by silica gel chromatography using 0-50% hexanes-ethyl acetate to afford as a colorless oil O1-benzyl O4-tert-butyl (5R)-6-hydroxy-5-methyl-1,4-diazepane-1,4-dicarboxylate, diastereomer 2 (2.1304 g, 59%). ¹H NMR (250 MHz, DMSO-d₆) δ 7.44-7.24 (m, 5H), 5.38-5.20 (m, 1H), 5.09 (s, 2H), 3.94-3.72 (m, 2H), 3.68-3.48 (m, 3H), 3.07-2.79 (m, 3H), 1.40 (s, 9H), 1.17-1.04 (m, 3H). ESI-MS m/z calc. 364.19983, found 365.1 (M+1)⁺; Retention time: 2.29 minutes; LC method T.

### Step 3: Benzyl (5R)-6-hydroxy-5-methyl-1,4-diazepane-1-carboxylate, diastereomer 2

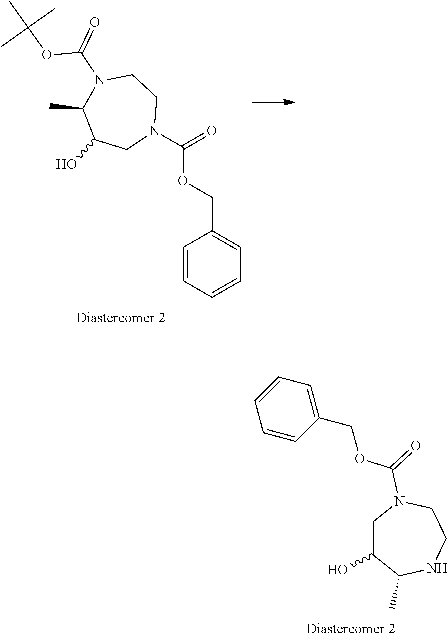 [SMILES: C[C@@H]1C(O)CN(C(=O)OCc2ccccc2)CCN1C(=O)OC(C)(C)C ; C[C@H]1NCCN(C(=O)OCc2ccccc2)CC1O]

A 100 mL round bottom flask was charged with 01-benzyl 04-tert-butyl (5R)-6-hydroxy-5-methyl-1,4-diazepane-1,4-dicarboxylate (926 mg, 2.541 mmol), DCM (8 mL) and HCl (7.5 mL of 4 M, 30.00 mmol) (4M dioxane solution). The mixture was stirred at rt for 2 hours. The volatiles were removed under reduced pressure. The solid was treated with DCM and hexanes and the solvents were evaporated. The operation was repeated 3 times. Drying in vacuo provided benzyl (5R)-6-hydroxy-5-methyl-1,4-diazepane-1-carboxylate (hydrochloride salt) (756 mg, 99%) as a white sticky resin. ESI-MS m/z calc. 264.1474, found 265.09 (M+1)⁺; Retention time: 0.62 minutes; (LC method A).

### Step 4: Benzyl (5R)-4-[3-[[4-chloro-6-(2,6-dimethylphenyl)pyrimidin-2-yl]sulfamoyl]benzoyl]-6-hydroxy-5-methyl-1,4-diazepane-1-carboxylate, diastereomer 2

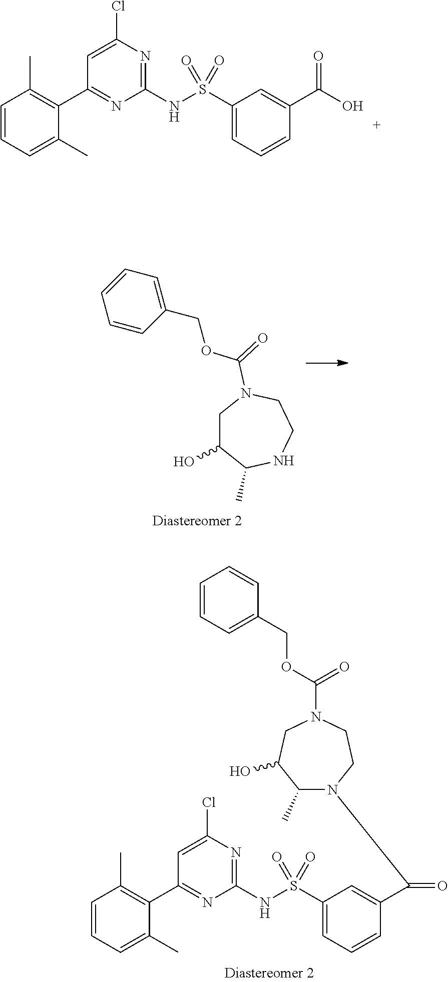

A 100 mL flask was charged under nitrogen with benzyl (5R)-6-hydroxy-5-methyl-1,4-diazepane-1-carboxylate (hydrochloride salt) (756 mg, 2.513 mmol), anhydrous DMF (14 mL) and 3-[[4-chloro-6-(2,6-dimethylphenyl)pyrimidin-2-yl]sulfamoyl]benzoic acid (0.893 g, 2.137 mmol). After dissolution of the reagents, the mixture was cooled down in an ice bath. DIEA (2.5 mL, 14.35 mmol) and HATU (1.027 g, 2.701 mmol) were added and the mixture was stirred at 0° C. for 30 min. The reaction was quenched by being poured in citric acid (75 mL of 10% w/v, 39.04 mmol)(10% aqueous) under vigorous stirring and cooled in ice. The resulting white solid was filtered. The wet solid was dissolved in DCM and the solution was dried over sodium sulfate. After concentration, it was purified by flash chromatography on silica gel (120 g column) using a gradient of methanol (0 to 10% over 60 min) in dichloromethane. The product eluted around 4-5% methanol. Evaporation of the solvents gave benzyl (5R)-4-[3-[[4-chloro-6-(2,6-dimethylphenyl)pyrimidin-2-yl]sulfamoyl]benzoyl]-6-hydroxy-5-methyl-1,4-diazepane-1-carboxylate (1.02 g, 72%) as a white foamy solid. ESI-MS m/z calc. 663.19183, found 664.23 (M+1)⁺; Retention time: 1.76 minutes (LC method A).

### Step 5: Benzyl (21R)-12-(2,6-dimethylphenyl)-21-methyl-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate, diastereomer 2 (Compound 113)

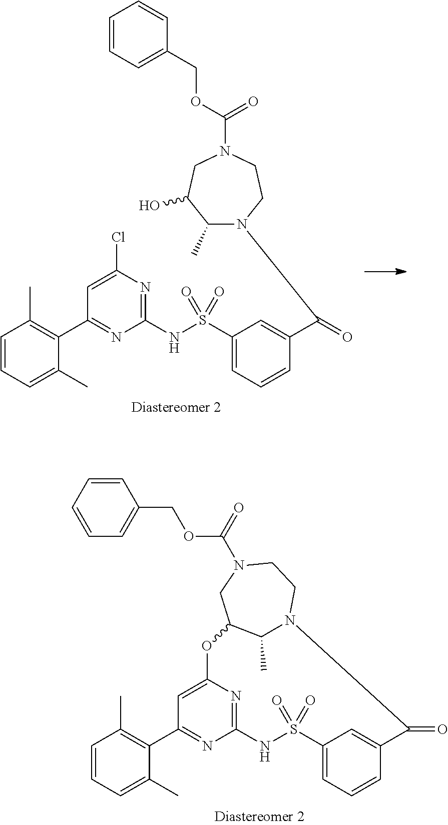 [SMILES: Cc1cccc(C)c1-c1cc(Cl)nc(NS(=O)(=O)c2cccc(C(=O)N3CCN(C(=O)OCc4ccccc4)CC(O)[C@H]3C)c2)n1 ; Cc1cccc(C)c1-c1cc2nc(n1)NS(=O)(=O)c1cccc(c1)C(=O)N1CCN(C(=O)OCc3ccccc3)CC(O2)[C@H]1C]

A 100 mL flask was charged under nitrogen with benzyl (5R)-4-[3-[[4-chloro-6-(2,6-dimethylphenyl)pyrimidin-2-yl]sulfamoyl]benzoyl]-6-hydroxy-5-methyl-1,4-diazepane-1-carboxylate (1.00 g, 1.506 mmol) and anhydrous DMF (50 mL). The mixture was cooled down in ice. NaH (546 mg of 60% w/w, 13.65 mmol) (60% mineral oil dispersion) was added in two equal portions, added 3 minutes after each other. The mixture was stirred under nitrogen at 0° C. for 1.5 h. The reaction mixture was slowly poured into an ice-cold citric acid (210 mL of 10% w/v, 109.3 mmol) aqueous solution under stirring. The resulting solid suspension was extracted with EtOAc (3×75 mL). After drying over sodium sulfate, evaporation of the solvents gave a residue that was dissolved in DCM and purified by flash chromatography on silica gel (80 g column) using a gradient of MeOH (0 to 5% over 30 min) in dichloromethane. The product eluted around 3-4% MeOH. Evaporation of the solvents and several cycle of trituration/evaporation in DCM/hexanes gave 123 mg of a solid that was 76% pure. The material was dissolved in DMSO (2.5 mL) and purified by reverse phase preparative HPLC (Cis) using a gradient of acetonitrile in water (1 to 99% over 15 min) and HCl as a modifier. The organic phase was evaporated and the solid that crashed out was extracted with DCM. Evaporation of the volatiles, trituration in DCM/hexanes and evaporation gave benzyl (21R)-12-(2,6-dimethylphenyl)-21-methyl-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (66 mg, 7%) as a white solid. ¹H NMR (499 MHz, DMSO-d₆) two conformers visible, ratio 70:30 δ 13.06 (broad s, 0.7H), 11.97 (br s, 0.3H), 8.03-7.94 (m, 1H), 7.89 (br s, 1H), 7.67-7.57 (m, 1H), 7.53 (s, 1H), 7.47-7.33 (m, 5H), 7.25 (br s, 1H), 7.12 (br s, 2H), 6.57-6.17 (br m, 1H), 5.44-4.90 (br m, 2H), 4.76 (s, 0.3H), 4.43 (s, 0.7H), 4.03 (dd, J=14.2, 5.1 Hz, 1H), 3.77 (br s, 1H), 3.68-3.35 (m, 3H), 3.14-2.79 (m, 2H), 2.24-1.81 (m, 6H), 1.15-0.97 (m, 3H). ESI-MS m/z calc. 627.21515, found 628.3 (M+1)⁺; Retention time: 1.58 minutes; LC method A.

### Example 54: Preparation of Compound 114

### Step 1: tert-Butyl 2-[benzyl-[(3R)-3-(tert-butoxycarbonylamino)-5-methyl-2-oxo-hexyl]amino]acetate

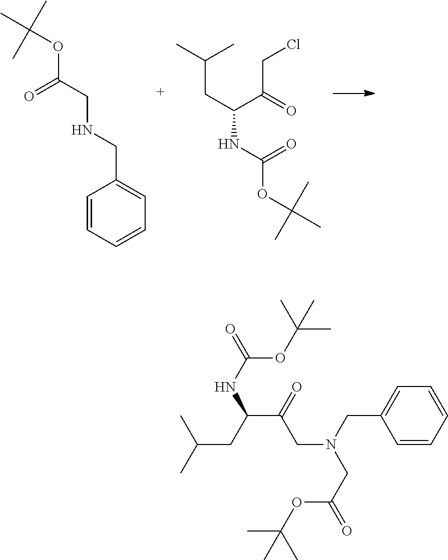 [SMILES: CC(C)(C)OC(=O)CNCc1ccccc1 ; CC(C)C[C@@H](NC(=O)OC(C)(C)C)C(=O)CCl ; CC(C)C[C@@H](NC(=O)OC(C)(C)C)C(=O)CN(CC(=O)OC(C)(C)C)Cc1ccccc1]

To a solution of tert-butyl 2-(benzylamino)acetate (12.04 g, 51.687 mmol) and tert-butyl N-[(1R)-1-(2-chloroacetyl)-3-methyl-butyl]carbamate (15.725 g, 56.637 mmol) in anhydrous DMF (110 mL) was added sodium bicarbonate (11.6 g, 138.08 mmol) followed by sodium iodide (5.08 g, 33.891 mmol). The resulting solution was stirred at ambient temperature for 17 hours before water (400 mL) was added. The solution was extracted with ether (2×400 mL). The combined organic layers were washed with brine (200 mL), dried over sodium sulfate, filtered and concentrated in vacuo to afford crude product. The crude product was purified by flash chromatography (loaded in DCM) (330 g silica gel, eluting 0 to 15% EtOAc/hexanes) to afford tert-butyl 2-[benzyl-[(3R)-3-(tert-butoxycarbonylamino)-5-methyl-2-oxo-hexyl]amino]acetate (22.85 g, 85%) as a pale-yellow liquid. ¹H NMR (250 MHz, CDCl₃) δ 7.66-6.79 (m, 5H), 5.12-4.85 (m, 1H), 4.40 (s, 1H), 3.96-3.76 (m, 2H), 3.76-3.54 (m, 2H), 3.36 (d, J=1.5 Hz, 2H), 1.91-1.52 (m, 2H), 1.52-1.33 (m, 18H), 1.31-1.23 (m, 1H), 0.94 (d, J=6.5 Hz, 3H), 0.88 (d, J=6.5 Hz, 3H). ESI-MS m/z calc. 448.29373, found 449.2 (M+1)⁺; Retention time: 5.04 minutes; LC method S.

### Step 2: tert-Butyl 2-[benzyl-[(2S,3R)-3-(tert-butoxycarbonylamino)-2-hydroxy-5-methyl-hexyl]amino]acetate and tert-butyl 2-[benzyl-[(2R,3R)-3-(tert-butoxycarbonylamino)-2-hydroxy-5-methyl-hexyl]amino]acetate

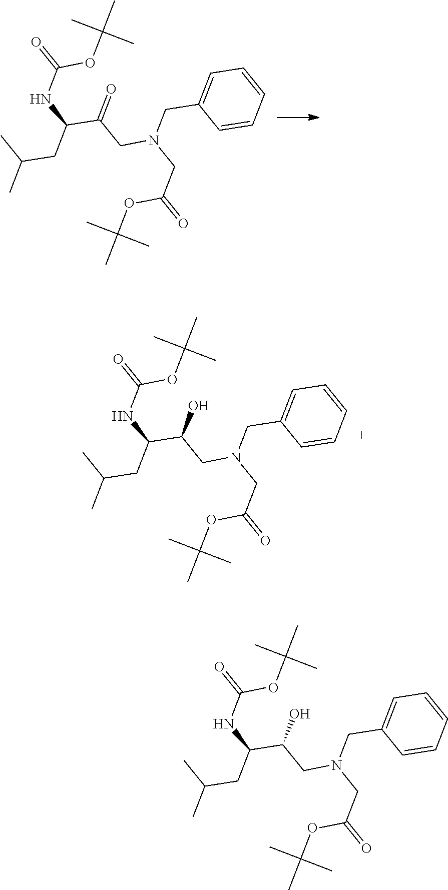 [SMILES: CC(C)C[C@@H](NC(=O)OC(C)(C)C)C(=O)CN(CC(=O)OC(C)(C)C)Cc1ccccc1 ; CC(C)C[C@@H](NC(=O)OC(C)(C)C)[C@@H](O)CN(CC(=O)OC(C)(C)C)Cc1ccccc1 ; CC(C)C[C@@H](NC(=O)OC(C)(C)C)[C@H](O)CN(CC(=O)OC(C)(C)C)Cc1ccccc1]

To a solution of tert-butyl 2-[benzyl-[(3R)-3-(tert-butoxycarbonylamino)-5-methyl-2-oxo-hexyl]amino]acetate (21.93 g, 48.886 mmol) in MeOH (220 mL) at 0° C. was added sodium borohydride (3.745 g, 98.989 mmol) (internal temperature<24° C.) The reaction solution at 0° C. for 20 minutes. Water (250 mL) was added. The solution was extracted with ethyl acetate (2×400 mL). The combined organic layers were dried over sodium sulfate, filtered and concentrated in vacuo. Benzene (2×100 mL) was added and concentrated in vacuo to remove a little water in crude product. The crude product was purified by flash chromatography (loaded in DCM) (330 g silica gel, eluting 0 to 20% EtOAc/hexanes) to afford tert-butyl 2-[benzyl-[(2S,3R)-3-(tert-butoxycarbonylamino)-2-hydroxy-5-methyl-hexyl]amino]acetate (15.13 g, 65%) (more polar) as a pale-yellow oil. ESI-MS m/z calc. 450.3094, found 451.2 (M+1)⁺; Retention time: 4.74 minutes (LC method S); ¹H NMR (250 MHz, CDCl₃) δ 7.51-7.06 (m, 5H), 4.63 (d, J=9.2 Hz, 1H), 3.87 (d, J=13.5 Hz, 1H), 3.69 (d, J=13.5 Hz, 1H), 3.64-3.46 (m, 2H), 3.19 (d, J=1.8 Hz, 2H), 2.84 (d, J=13.1 Hz, 1H), 2.54 (dd, J=13.0, 9.8 Hz, 1H), 1.78-1.54 (m, 1H), 1.44 (s, 9H), 1.43 (s, 9H), 1.42-1.27 (m, 2H), 0.91 (d, J=4.3 Hz, 3H), 0.89 (d, J=4.3 Hz, 3H); and tert-butyl 2-[benzyl-[(2R,3R)-3-(tert-butoxycarbonylamino)-2-hydroxy-5-methyl-hexyl]amino]acetate (4.36 g, 19%) (less polar) as a pale-yellow oil, ESI-MS m/z calc. 450.3094, found 451.2 (M+1)⁺; Retention time: 4.73 minutes (LC method S); ¹H NMR (250 MHz, CDCl₃) δ 7.48-7.04 (m, 5H), 4.76 (d, J=9.9 Hz, 1H), 3.89 (d, J=13.5 Hz, 1H), 3.77-3.32 (m, 4H), 3.19 (d, J=5.2 Hz, 2H), 2.73 (dd, J=13.1, 3.2 Hz, 1H), 2.52 (dd, J=13.1, 10.7 Hz, 1H), 1.78-1.49 (m, 3H), 1.44 (s, 9H), 1.40 (s, 9H), 1.07-0.69 (m, 6H).

### Step 3: (6S,7R)-4-Benzyl-6-hydroxy-7-isobutyl-1,4-diazepan-2-one

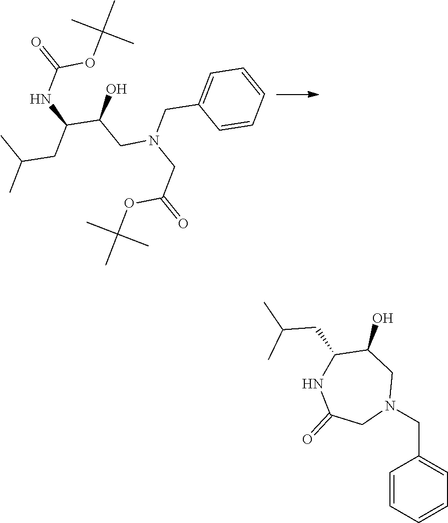 [SMILES: CC(C)C[C@@H](NC(=O)OC(C)(C)C)[C@@H](O)CN(CC(=O)OC(C)(C)C)Cc1ccccc1 ; CC(C)C[C@H]1NC(=O)CN(Cc2ccccc2)C[C@@H]1O]

tert-Butyl 2-[benzyl-[(2S,3R)-3-(tert-butoxycarbonylamino)-2-hydroxy-5-methyl-hexyl]amino]acetate (15.13 g, 33.577 mmol) was added HCl (335 mL of 4 M, 1.3400 mol). The resulting solution was stirred at ambient temperature for 24 hours. Then all solvents were removed under reduced pressure. The residue was dissolved in anhydrous EtOH (700 mL). The resulting solution was stirred at 50° C. for 21 hours. Then TEA (33.977 g, 46.800 mL, 335.77 mmol) was added and continued to stir for 7.5 hours at 50° C. All solvents were removed under reduced pressure. The residue was dissolved in ethyl acetate (800 mL) and washed with saturated sodium bicarbonate aqueous solution (200 mL). The organic layer was separated, and aqueous layer was extracted with ethyl acetate (200 mL). The combined organic layers were dried over sodium sulfate, filtered and concentrated under reduced pressure. The crude product was purified by flash chromatography (loaded in DCM) (220 g silica gel, eluting 0 to 100% EtOAc/hexanes) to afford (6S,7R)-4-benzyl-6-hydroxy-7-isobutyl-1,4-diazepan-2-one (7.33 g, 77%) as a white solid. ESI-MS m/z calc. 276.18378, found 277.2 (M+1)⁺; Retention time: 2.17 minutes; LC method S.

### Step 4: tert-Butyl (6S,7R)-4-benzyl-6-hydroxy-7-isobutyl-1,4-diazepane-1-carboxylate

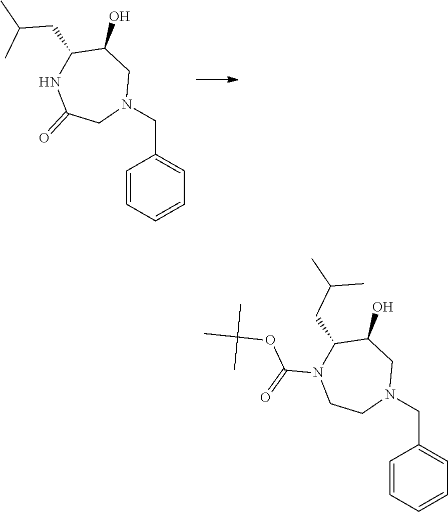 [SMILES: CC(C)C[C@H]1NC(=O)CN(Cc2ccccc2)C[C@@H]1O ; CC(C)C[C@@H]1[C@@H](O)CN(Cc2ccccc2)CCN1C(=O)OC(C)(C)C]

To a solution of (6S,7R)-4-benzyl-6-hydroxy-7-isobutyl-1,4-diazepan-2-one (7.33 g, 25.673 mmol) in anhydrous THF (260 mL) was added LAH (5.86 g, 154.40 mmol) very slowly. The suspension solution was heated at 40° C. under argon for 16 hours. The reaction solution was cooled to 0° C., then water (5.9 mL) was added dropwise followed by the addition of 15% of NaOH aqueous solution (5.9 mL) and water (17.7 mL). THE (200 mL) was added and suspension solution was stirred at ambient temperature for 1 hour. The suspension was filtered through Celite and washed with THE (100 mL). The filtrate was concentrated under reduced pressure to afford crude amino alcohol intermediate as a colorless liquid which was dissolved in a mixture of dioxane (130 mL) and a sodium bicarbonate saturated aqueous solution (130 mL). Boc anhydride (5.6 g, 25.659 mmol) was added and the resulting solution was stirred at ambient temperature 16 hours. Water (100 mL) and ethyl acetate (200 mL) were added. The organic layer was separated, and aqueous layer was extracted with ethyl acetate (2×200 mL). The combined organic layers were dried over anhydrous sodium sulfate, filtered and concentrated under reduced pressure. The crude product was purified by flash chromatography (loaded in hexane) (330 g silica gel, eluting 0 to 30% EtOAc) to afford tert-butyl (6S,7R)-4-benzyl-6-hydroxy-7-isobutyl-1,4-diazepane-1-carboxylate (6.5889 g, 68%) as a white solid. ¹H NMR (250 MHz, DMSO-d₆) δ 7.48-7.05 (m, 5H), 5.00 (dd, J=16.2, 6.3 Hz, 1H), 3.99-3.35 (m, 5H), 2.91-2.53 (m, 3H), 2.23 (dt, J=12.3, 8.4 Hz, 2H), 1.61-1.17 (m, 12H), 1.00-0.71 (m, 6H). ESI-MS m/z calc. 362.25696, found 363.3 (M+1)⁺; Retention time: 1.86 minutes; LC method T.

### Step 5: tert-Butyl (6S,7R)-6-hydroxy-7-isobutyl-1,4-diazepane-1-carboxylate

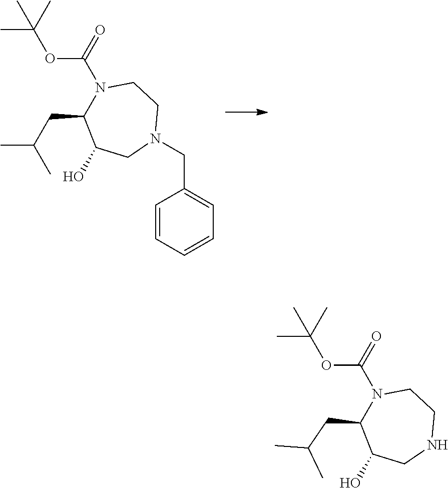 [SMILES: CC(C)C[C@@H]1[C@@H](O)CN(Cc2ccccc2)CCN1C(=O)OC(C)(C)C ; CC(C)C[C@@H]1[C@@H](O)CNCCN1C(=O)OC(C)(C)C]

A 100 mL round bottom flask was charged with tert-butyl (6S,7R)-4-benzyl-6-hydroxy-7-isobutyl-1,4-diazepane-1-carboxylate (1.51 g, 4.165 mmol) and MeOH (30 mL). The solution was sparged with nitrogen for 10 minutes. Pd(OH)₂ (640 mg of 20% w/w, 0.9115 mmol) (20% wet) was added and the reaction was stirred at room temperature under hydrogen (balloon) for 15 hours. The solution was sparged with nitrogen for 10 minutes and filtered twice through a pad of Celite. After concentration, the solution was microfiltered through a filter disc and the solvent was evaporated to give tert-butyl (6S,7R)-6-hydroxy-7-isobutyl-1,4-diazepane-1-carboxylate (1.111 g, 98%) as an off-white solid. ESI-MS m/z calc. 272.21, found 273.2 (M+1)⁺; Retention time: 0.94 minutes (LC method A).

### Step 6: tert-Butyl (6S,7R)-4-[3-[[4-chloro-6-(2,6-dimethylphenyl)pyrimidin-2-yl]sulfamoyl]benzoyl]-6-hydroxy-7-isobutyl-1,4-diazepane-1-carboxylate

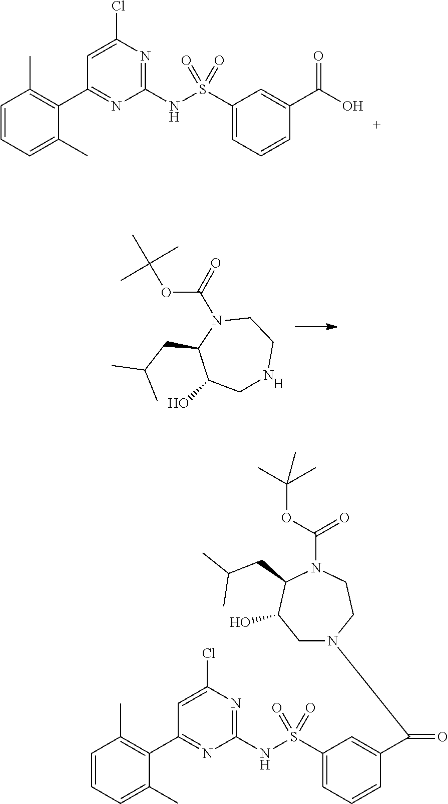

A 100 mL flask was charged under nitrogen with tert-butyl (6S,7R)-6-hydroxy-7-isobutyl-1,4-diazepane-1-carboxylate (1.11 g, 4.075 mmol), anhydrous DMF (20 mL) and 3-[[4-chloro-6-(2,6-dimethylphenyl)pyrimidin-2-yl]sulfamoyl]benzoic acid (1.42 g, 3.398 mmol). After dissolution of the reagents, the mixture was cooled down in an ice bath. DIEA (4 mL, 22.96 mmol) and HATU (1.59 g, 4.182 mmol) were added and the mixture was stirred at 0° C. for 20 min. The reaction was quenched by being poured in citric acid (120 mL of 10% w/v, 62.46 mmol) (10% aqueous) under vigorous stirring and cooled in ice. The resulting white solid was filtered. The wet solid was dissolved in DCM and the solution was dried over sodium sulfate. After concentration, it was purified by flash chromatography on silica gel (120 g column) using a gradient of methanol (0 to 5% over 30 min) in dichloromethane. The product eluted around 3-4% methanol. Evaporation of the solvents gave tert-butyl (6S,7R)-4-[3-[[4-chloro-6-(2,6-dimethylphenyl)pyrimidin-2-yl]sulfamoyl]benzoyl]-6-hydroxy-7-isobutyl-1,4-diazepane-1-carboxylate (1.853 g, 81%) as a white foamy solid. ESI-MS m/z calc. 671.25446, found 672.35 (M+1)⁺; Retention time: 2.06 minutes (LC method A).

### Step 7: tert-Butyl (16S,17R)-12-(2,6-dimethylphenyl)-17-(2-methylpropyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (Compound 114)

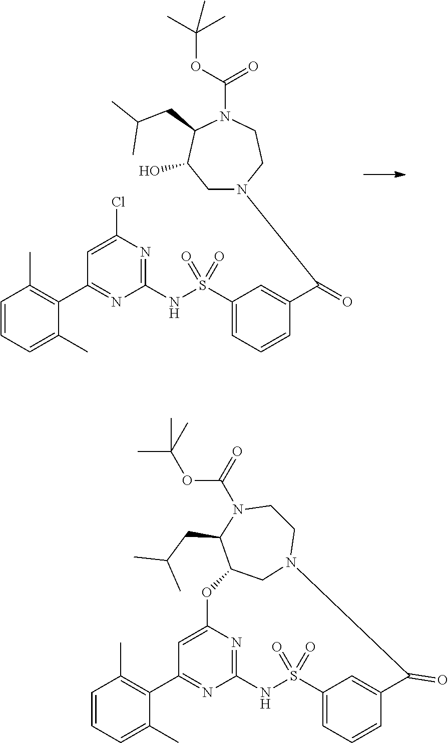 [SMILES: Cc1cccc(C)c1-c1cc(Cl)nc(NS(=O)(=O)c2cccc(C(=O)N3CCN(C(=O)OC(C)(C)C)[C@H](CC(C)C)[C@@H](O)C3)c2)n1 ; Cc1cccc(C)c1-c1cc2nc(n1)NS(=O)(=O)c1cccc(c1)C(=O)N1CCN(C(=O)OC(C)(C)C)[C@H](CC(C)C)[C@H](C1)O2]

A 100 mL flask was charged under nitrogen with tert-butyl (6S,7R)-4-[3-[[4-chloro-6-(2,6-dimethylphenyl)pyrimidin-2-yl]sulfamoyl]benzoyl]-6-hydroxy-7-isobutyl-1,4-diazepane-1-carboxylate (1.04 g, 1.547 mmol) and anhydrous DMF (50 mL). The mixture was cooled down in ice. NaH (550 mg of 60% w/w, 13.75 mmol) (60% mineral oil dispersion) was added in two equal portions, added 3 minutes after each other. The mixture was stirred under nitrogen at 0° C. for 10 minutes. The ice bath was removed, and the reaction was vigorously stirred under nitrogen for 1.5 hours. The reaction mixture was slowly poured into an ice-cold citric acid (220 mL of 10% w/v, 114.5 mmol) aqueous solution under stirring. The resulting solid suspension was extracted with EtOAc (3×75 mL). After drying over sodium sulfate, evaporation of the solvents gave a residue that was dissolved in DCM and purified by flash chromatography on silica gel (80 g column) using a gradient of MeOH (0 to 5% over 30 min) in dichloromethane. The product eluted around 3-4% MeOH. Evaporation of the solvents and several cycles of trituration/evaporation in DCM/hexanes gave tert-butyl (16S,17R)-12-(2,6-dimethylphenyl)-17-(2-methylpropyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (341 mg, 34%) as a white solid. ¹H NMR (499 MHz, DMSO-d₆) several rotamers visible δ 13.36-11.65 (br m, 1H), 8.58-8.30 (m, 1H), 7.88 (broad s, 1H), 7.64 (br s, 2H), 7.25 (t, J=7.7 Hz, 1H), 7.12 (d, J=7.6 Hz, 2H), 6.29 (br s, 1H), 5.94-5.20 (m, 1H), 4.74-4.00 (m, 2H), 3.97-3.36 (m, 2H), 3.24-2.80 (m overlapped with water, 2H), 2.03 (br s, 6H), 1.89-1.74 (m, 1H), 1.73-1.19 (m, 12H), 0.97-0.71 (m, 6H). ESI-MS m/z calc. 635.2778, found 636.37 (M+1)⁺; Retention time: 1.9 minutes; LC method A.

### Example 55: Preparation of Compound 115

### Step 1: (6R,7R)-4-Benzyl-6-hydroxy-7-isobutyl-1,4-diazepan-2-one

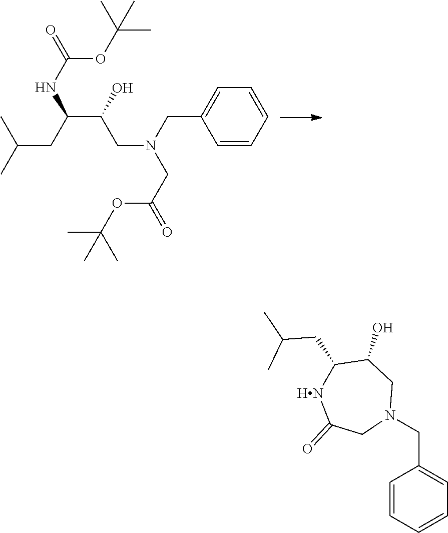

tert-butyl 2-[benzyl-[(2R,3R)-3-(tert-butoxycarbonylamino)-2-hydroxy-5-methyl-hexyl]amino]acetate (4.36 g, 9.6758 mmol) was added HCl (100 mL of 4 M in dioxane, 400.00 mmol). The resulting solution was stirred at ambient temperature for 24 hours. Then all solvents were removed under reduced pressure. The residue was dissolved in anhydrous EtOH (200 mL). The resulting solution was stirred at 50° C. for 6 hours. Then TEA (9.8010 g, 13.5 mL, 96.857 mmol) was added and reaction continued for 15 hours at 50° C. All solvents were removed under reduced pressure. The residue was dissolved in ethyl acetate (500 mL) and washed with saturated sodium bicarbonate aqueous solution (100 mL). The organic layer was separated, and aqueous layer was extracted with ethyl acetate (100 mL). The combined organic layers were dried over sodium sulfate, filtered and concentrated under reduced pressure. The crude product was purified by flash chromatography (loaded in DCM) (120 g silica gel, eluting 0 to 100% EtOAc/hexanes) to afford (6R,7R)-4-benzyl-6-hydroxy-7-isobutyl-1,4-diazepan-2-one (2.19 g, 81%) as a white foam solid. ESI-MS m/z calc. 276.18378, found 277.1 (M+1)⁺; Retention time: 2.08 minutes; LC method S.

### Step 2: tert-Butyl (6R,7R)-4-benzyl-6-hydroxy-7-isobutyl-1,4-diazepane-1-carboxylate

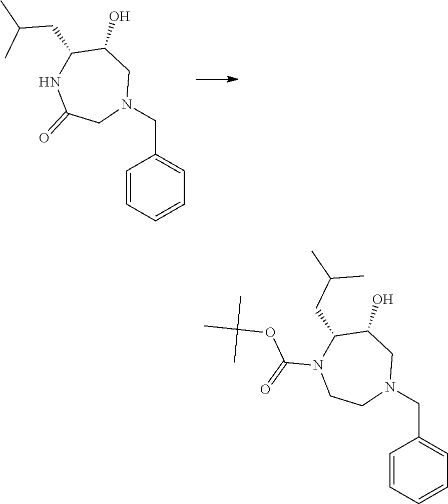 [SMILES: CC(C)C[C@H]1NC(=O)CN(Cc2ccccc2)C[C@H]1O ; CC(C)C[C@@H]1[C@H](O)CN(Cc2ccccc2)CCN1C(=O)OC(C)(C)C]

To a solution of (6R,7R)-4-benzyl-6-hydroxy-7-isobutyl-1,4-diazepan-2-one (2.19 g, 7.5595 mmol) in anhydrous THE (76 mL) was added LAH (1.72 g, 45.318 mmol) very slowly. The suspension solution was heated at 40° C. under argon for 16 hours. The reaction solution was cooled to 0° C., then water (1.7 mL) was added dropwise followed by the addition of 15% of NaOH aqueous solution (1.7 mL) and water (5.1 mL). THE (80 mL) was added and suspension solution was stirred at ambient temperature for 1 hour. The suspension was filtered through Celite and washed with THE (100 mL). The filtrate was concentrated under reduced pressure to afford a crude amino alcohol intermediate as a colorless liquid which was dissolved in a mixture of dioxane (40 mL) and a sodium bicarbonate saturated aqueous solution (40 mL). Then Boc anhydride (2.09 g, 9.5763 mmol) was added and the solution was stirred at ambient temperature for 16 hours. Then additional Boc anhydride (0.43 g, 1.9702 mmol) was added and the reaction was stirred for additional 8 hour. Water (50 mL) and ethyl acetate (100 mL) were added. The organic layer was separated, and aqueous layer was extracted with ethyl acetate (2×100 mL). The combined organic layers were dried over anhydrous sodium sulfate, filtered and concentrated under reduced pressure. The crude product was purified by flash chromatography (loaded in hexanes) (120 g silica gel, eluting 0 to 15% EtOAc/hexanes) to afford tert-butyl (6R,7R)-4-benzyl-6-hydroxy-7-isobutyl-1,4-diazepane-1-carboxylate (1.9399 g, 65%) as a colorless liquid. ¹H NMR (250 MHz, DMSO-d₆) δ 7.48-7.09 (m, 5H), 4.35 (s, 1H), 4.13-3.74 (m, 2H), 3.65 (t, J=2.6 Hz, 2H), 3.52 (d, J=15.1 Hz, 1H), 3.14-2.75 (m, 2H), 2.68 (d, J=12.3 Hz, 1H), 2.46-2.10 (m, 2H), 1.69-1.44 (m, 2H), 1.43-1.21 (m, 10H), 0.95-0.77 (m, 6H). ESI-MS m/z calc. 362.25696, found 363.6 (M+1)⁺; Retention time: 1.86 minutes; LC method T.

### Step 3: tert-Butyl (6R,7R)-6-hydroxy-7-isobutyl-1,4-diazepane-1-carboxylate

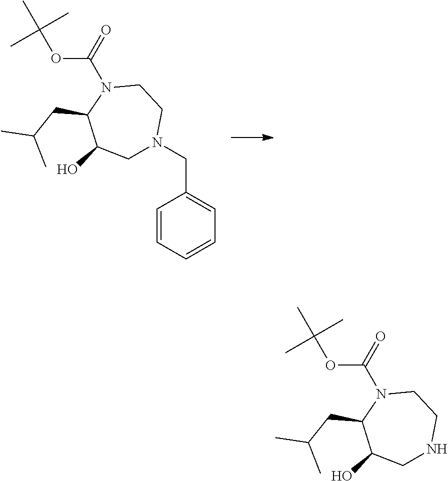 [SMILES: CC(C)C[C@@H]1[C@H](O)CN(Cc2ccccc2)CCN1C(=O)OC(C)(C)C ; CC(C)C[C@@H]1[C@H](O)CNCCN1C(=O)OC(C)(C)C]

A 100 mL round bottom flask was charged with tert-butyl (6R,7R)-4-benzyl-6-hydroxy-7-isobutyl-1,4-diazepane-1-carboxylate (1.92 g, 5.296 mmol) and MeOH (35 mL). The solution was sparged with nitrogen for 10 minutes. Pd(OH)₂ (500 mg of 20% w/w, 0.7121 mmol) (20% wet) was added and the reaction was stirred at room temperature under hydrogen (balloon) for 2 days. The solution was sparged with nitrogen for 10 minutes and filtered twice through a pad of Celite. After concentration, the solution was microfiltered and the solvent was evaporated to give tert-butyl (6R,7R)-6-hydroxy-7-isobutyl-1,4-diazepane-1-carboxylate (1.43 g, 99%) as a pale brown resin. ESI-MS m/z calc. 272.21, found 273.2 (M+1)⁺; Retention time: 0.98 minutes; LC method A.

### Step 4: tert-Butyl (6R,7R)-4-[3-[[4-chloro-6-(2,6-dimethylphenyl)pyrimidin-2-yl]sulfamoyl]benzoyl]-6-hydroxy-7-isobutyl-1,4-diazepane-1-carboxylate

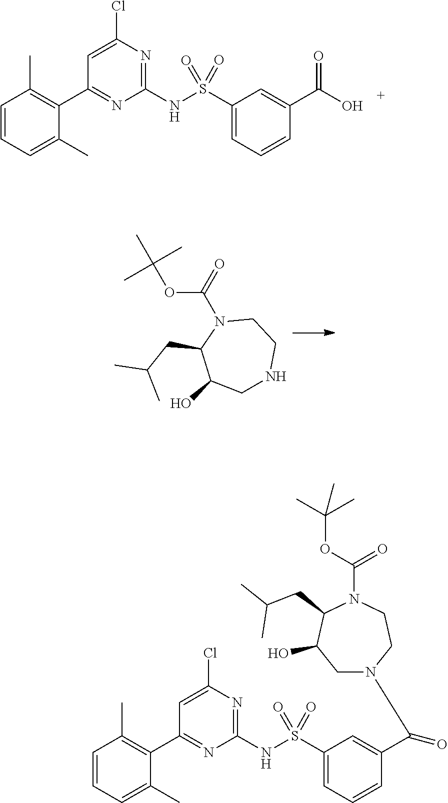 [SMILES: Cc1cccc(C)c1-c1cc(Cl)nc(NS(=O)(=O)c2cccc(C(=O)O)c2)n1 ; CC(C)C[C@@H]1[C@H](O)CNCCN1C(=O)OC(C)(C)C ; Cc1cccc(C)c1-c1cc(Cl)nc(NS(=O)(=O)c2cccc(C(=O)N3CCN(C(=O)OC(C)(C)C)[C@H](CC(C)C)[C@H](O)C3)c2)n1]

A 100 mL flask was charged under nitrogen with tert-butyl (6R,7R)-6-hydroxy-7-isobutyl-1,4-diazepane-1-carboxylate (1.43 g, 5.250 mmol), anhydrous DMF (25 mL) and 3-[[4-chloro-6-(2,6-dimethylphenyl)pyrimidin-2-yl]sulfamoyl]benzoic acid (1.854 g, 4.437 mmol). After dissolution of the reagents, the mixture was cooled down in an ice bath. DIEA (5.2 mL, 29.85 mmol) and HATU (2.14 g, 5.628 mmol) were added and the mixture was stirred at 0° C. for 45 min. The reaction was quenched by being poured in citric acid (160 mL of 10% w/v, 83.28 mmol) (10% aqueous) under vigorous stirring and cooled in ice. The resulting white solid was filtered and dried. The solid was dissolved in DCM. After concentration, it was purified by flash chromatography on silica gel (220 g column) using a gradient of methanol (0 to 5% over 30 min) in dichloromethane. The product eluted around 3-4% methanol. Evaporation of the solvents gave tert-butyl (6R,7R)-4-[3-[[4-chloro-6-(2,6-dimethylphenyl)pyrimidin-2-yl]sulfamoyl]benzoyl]-6-hydroxy-7-isobutyl-1,4-diazepane-1-carboxylate (1.575 g, 53%) as a white foamy solid. ESI-MS m/z calc. 671.25446, found 672.33 (M+1)⁺; Retention time: 2.0 minutes; LC method A.

### Step 5: tert-Butyl (16R,17R)-12-(2,6-dimethylphenyl)-17-(2-methylpropyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (Compound 115)

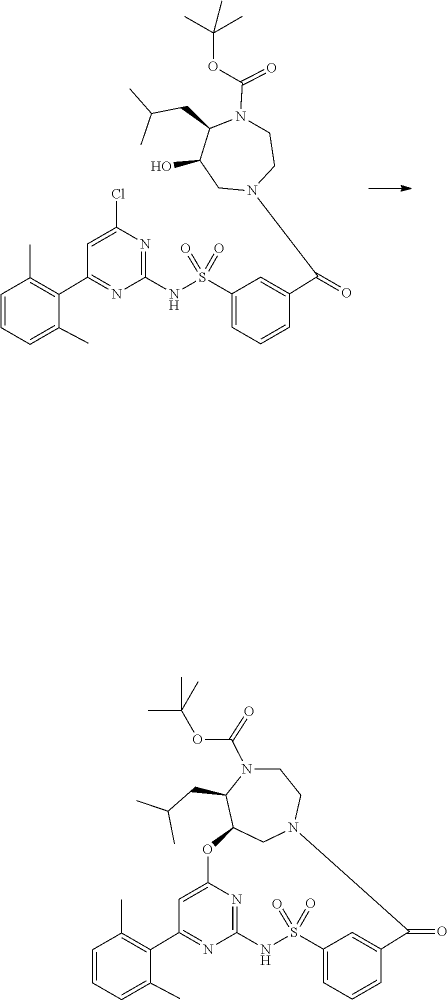 [SMILES: Cc1cccc(C)c1-c1cc(Cl)nc(NS(=O)(=O)c2cccc(C(=O)N3CCN(C(=O)OC(C)(C)C)[C@H](CC(C)C)[C@H](O)C3)c2)n1 ; Cc1cccc(C)c1-c1cc2nc(n1)NS(=O)(=O)c1cccc(c1)C(=O)N1CCN(C(=O)OC(C)(C)C)[C@H](CC(C)C)[C@@H](C1)O2]

A 100 mL flask was charged under nitrogen with tert-butyl (6R,7R)-4-[3-[[4-chloro-6-(2,6-dimethylphenyl)pyrimidin-2-yl]sulfamoyl]benzoyl]-6-hydroxy-7-isobutyl-1,4-diazepane-1-carboxylate (1.062 g, 1.580 mmol) and anhydrous DMF (50 mL). The mixture was cooled down in ice. NaH (563 mg of 60% w/w, 14.08 mmol) (60% mineral oil dispersion) was added in two equal portions, added 3 minutes after each other. The mixture was stirred under nitrogen at 0° C. for 10 minutes. The ice bath was removed, and the reaction was vigorously stirred under nitrogen for 1.5 hours. The reaction mixture was slowly poured into an ice-cold citric acid (230 mL of 10% w/v, 119.7 mmol) aqueous solution under stirring. The resulting solid suspension was extracted with EtOAc (3×75 mL). After drying over sodium sulfate, evaporation of the solvents gave a residue that was dissolved in DCM and purified by flash chromatography on silica gel (120 g column) using a gradient of MeOH (0 to 5% over 30 min) in dichloromethane. The product eluted around 3-4% MeOH. Evaporation of the solvents and several cycles of trituration/evaporation in DCM/hexanes gave tert-butyl (16R,17R)-12-(2,6-dimethylphenyl)-17-(2-methylpropyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (692 mg, 67%) as a white solid. ¹H NMR (500 MHz, DMSO-d₆) mixture of rotamers (60:40) δ 13.12-11.88 (broad m, 1H), 8.35 (s, 0.6H), 8.28 (s, 0.4H), 7.89 (broad s, 1H), 7.65 (br s, 2H), 7.25 (t, J=7.9 Hz, 1H), 7.12 (d, J=7.6 Hz, 2H), 6.31 (bt s, 1H), 5.70-5.44 (m, 1H), 4.70-4.30 (m, 2H), 4.08-3.84 (m, 1H), 3.44 (q, J=13.2 Hz, 1H), 3.31-3.18 (m, 2H overlapped with water), 3.15-3.02 (m, 1H), 2.07-1.89 (m, 7H), 1.57-1.53 (m, 2H), 1.47 and 1.42 (2 d, total 9H), 1.02-0.77 (m, 6H). ESI-MS m/z calc. 635.2778, found 636.33 (M+1)⁺; Retention time: 1.85 minutes; LC method A.

### Example 56: Preparation of Compound 116

### Step 1: (7S)-4-Benzyl-6-hydroxy-7-isobutyl-1,4-diazepan-2-one

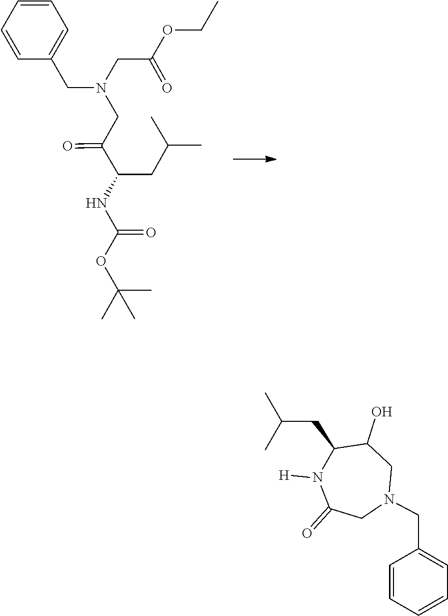 [SMILES: CCOC(=O)CN(CC(=O)[C@H](CC(C)C)NC(=O)OC(C)(C)C)Cc1ccccc1 ; CC(C)C[C@@H]1NC(=O)CN(Cc2ccccc2)CC1O]

To a solution of ethyl 2-[benzyl-[(3S)-3-(tert-butoxycarbonylamino)-5-methyl-2-oxo-hexyl]amino]acetate (52.3 g, 124.36 mmol) in MeOH (620 mL) at −70° C. (internal temperature) was added sodium borohydride (9 g, 237.89 mmol) in 3 portions, then stirred at this temperature for 1 hour before it was placed at −80° C. refrigerator. The reaction was quenched with saturated ammonium chloride aqueous solution (650 mL), then warmed up to ambient temperature. The reaction solution was extracted with ether (3×700 mL). The combined organic layers were washed with brine (100 mL), dried over sodium sulfate, filtered and concentrated under reduced pressure. The crude product obtained was dissolved in the mixture of DCM (310 mL) and HCl (310.90 mL of 4 M, 1.2436 mol) in 1,4-dioxane. The resulting solution was stirred at ambient temperature for 50 minutes. All solvents were removed under reduced pressure. Residue obtained was dissolved in anhydrous EtOH (2500 mL), heated at 40° C. for 15 hours, then at 70° C. for 24 hours. The reaction solution was cooled to 50° C., and TEA (126.88 g, 175 mL, 1.2539 mol) was added. The reaction solution continued to stir for 5 hours at this temperature. All solvents were removed under reduced pressure. The residue was dissolved in ethyl acetate (1200 mL) and washed with saturated sodium bicarbonate aqueous solution (600 mL). The organic layer was separated, and aqueous layer was extracted with ethyl acetate (600 mL). The combined organic layers were dried over sodium sulfate, filtered and concentrated under reduced pressure. The crude product was purified by flash chromatography (loaded in DCM) (330 g silica gel, eluting 0 to 100% EtOAc/hexanes) to afford (7S)-4-benzyl-6-hydroxy-7-isobutyl-1,4-diazepan-2-one (30.52 g, 89%) as a brown foam solid, mixture of syn/anti isomers. ESI-MS m/z calc. 276.18378, found 277.2 (M+1)⁺; Retention time: 2.08 minutes; LC method S.

### Step 2: tert-Butyl (6S,7S)-4-benzyl-6-hydroxy-7-isobutyl-1,4-diazepane-1-carboxylate and tert-butyl (6R,7S)-4-benzyl-6-hydroxy-7-isobutyl-1,4-diazepane-1-carboxylate

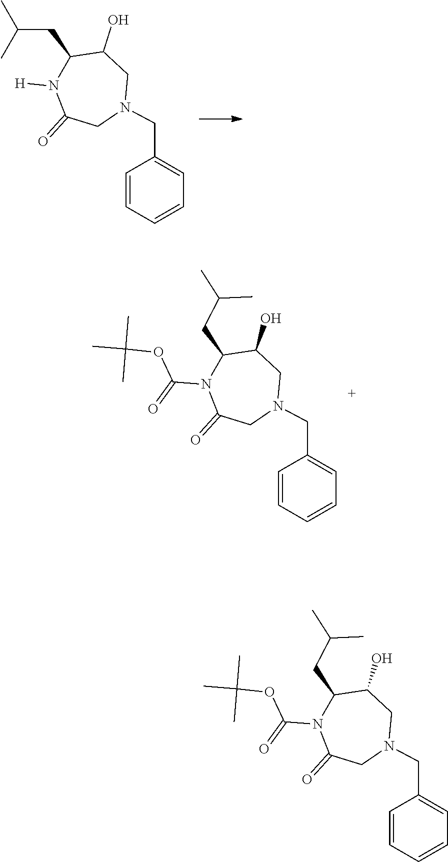 [SMILES: CC(C)C[C@@H]1NC(=O)CN(Cc2ccccc2)CC1O ; CC(C)C[C@H]1[C@@H](O)CN(Cc2ccccc2)CC(=O)N1C(=O)OC(C)(C)C ; CC(C)C[C@H]1[C@H](O)CN(Cc2ccccc2)CC(=O)N1C(=O)OC(C)(C)C]

To a solution of (7S)-4-benzyl-6-hydroxy-7-isobutyl-1,4-diazepan-2-one (30.52 g, 110.43 mmol) in anhydrous THE (1100 mL) was added LAH (25.3 g, 666.59 mmol) in small portions carefully. Then the reaction was stirred at 40° C. for 22 hours, then the reaction was cooled to 0° C. Water (25.3 mL) was added dropwise followed by 15% NaOH aqueous solution (25.3 mL) and water (75.9 mL), then THF (300 mL) was added and the resulting solution was stirred at ambient temperature for 1 hour. Solution was filtered through Celite and the pad was washed with THF. The filtrate was concentrated under reduced pressure to afford crude aminol intermediate as a pale-yellow liquid which was dissolved in a mixture of dioxane (550 mL) and aqueous sodium bicarbonate (550 mL). Boc anhydride (32.4 g, 144.00 mmol) was added. The resulting solution was stirred at ambient temperature for 19 hours. Water (100 mL) and ethyl acetate (200 mL) were added, and organic layer was separated. The aqueous layer was extracted with ethyl acetate (2×500 mL). The combined organic layers were washed with brine (150 mL), dried over anhydrous sodium sulfate, filtered and concentrated under reduced pressure. The crude product was purified by flash chromatography (loaded in DCM) (330 g silica gel, eluting 0 to 30% EtOAc/hexanes) to afford tert-butyl (6S,7S)-4-benzyl-6-hydroxy-7-isobutyl-1,4-diazepane-1-carboxylate (8.212 g, 19%)(less polar) as a pale-yellow oil. ESI-MS m/z calc. 362.2569, found 363.3 (M+1)⁺, Retention time: 1.88 minutes (LC method W), ¹H NMR (250 MHz, DMSO-d₆) δ 7.55-7.08 (m, 5H), 4.36 (s, 1H), 4.08-3.73 (m, 2H), 3.66 (s, 2H), 3.52 (d, J=15.0 Hz, 1H), 3.10-2.76 (m, 2H), 2.76-2.59 (m, 1H), 2.45-2.20 (m, 2H), 1.63-1.45 (m, 2H), 1.38 (d, J=6.5 Hz, 9H), 1.31-1.18 (m, 1H), 0.99-0.77 (m, 6H); and tert-butyl (6R,7S)-4-benzyl-6-hydroxy-7-isobutyl-1,4-diazepane-1-carboxylate (20.275 g, 49%) (more polar) as a white foam solid, ESI-MS m/z calc. 362.2569, found 363.3 (M+1)⁺; Retention time: 1.88 minutes (LC method W), ¹H NMR (250 MHz, DMSO-d₆) δ 7.29 (m, J 5.5 Hz, 5H), 5.00 (dd, J=16.1, 6.3 Hz, 1H), 3.89-3.38 (m, 5H), 2.84-2.56 (m, 3H), 2.33-2.14 (m, 2H), 1.39 (m, 12H), 1.01-0.72 (m, 6H).

### Step 3: tert-Butyl (6R,7S)-6-hydroxy-7-isobutyl-1,4-diazepane-1-carboxylate

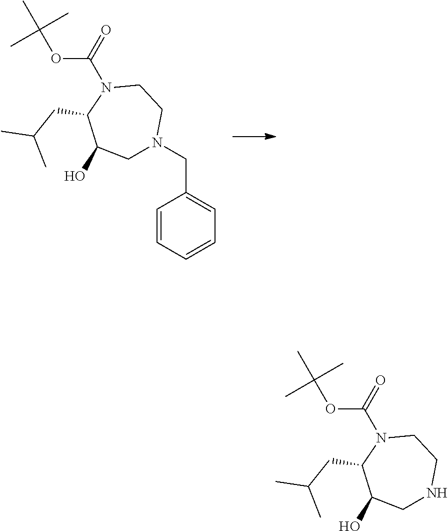 [SMILES: CC(C)C[C@H]1[C@H](O)CN(Cc2ccccc2)CCN1C(=O)OC(C)(C)C ; CC(C)C[C@H]1[C@H](O)CNCCN1C(=O)OC(C)(C)C]

A 100 mL round bottom flask was charged with tert-butyl (6R,7S)-4-benzyl-6-hydroxy-7-isobutyl-1,4-diazepane-1-carboxylate (1.63 g, 4.496 mmol) and MeOH (30 mL). The solution was sparged with nitrogen for 10 minutes. Pd(OH)₂ (500 mg of 20% w/w, 0.7121 mmol) (20% wet) was added and the reaction was stirred at room temperature under hydrogen (balloon) for 15 hours (70-80% conversion). Another load of catalyst Pd(OH)₂ (220 mg of 20% w/w, 0.3133 mmol) was added and a new balloon was installed. After 4 days, the solution was sparged with nitrogen for 10 minutes and filtered twice through a pad of Celite. After concentration, The solution was microfiltered and the solvent was evaporated to give tert-butyl (6R,7S)-6-hydroxy-7-isobutyl-1,4-diazepane-1-carboxylate (1.227 g, 99%) as a tan solid. ESI-MS m/z calc. 272.21, found 273.2 (M+1)⁺; Retention time: 1.03 minutes; LC method A.

### Step 4: tert-Butyl (6R,7S)-4-[3-[[4-chloro-6-(2,6-dimethylphenyl)pyrimidin-2-yl]sulfamoyl]benzoyl]-6-hydroxy-7-isobutyl-1,4-diazepane-1-carboxylate

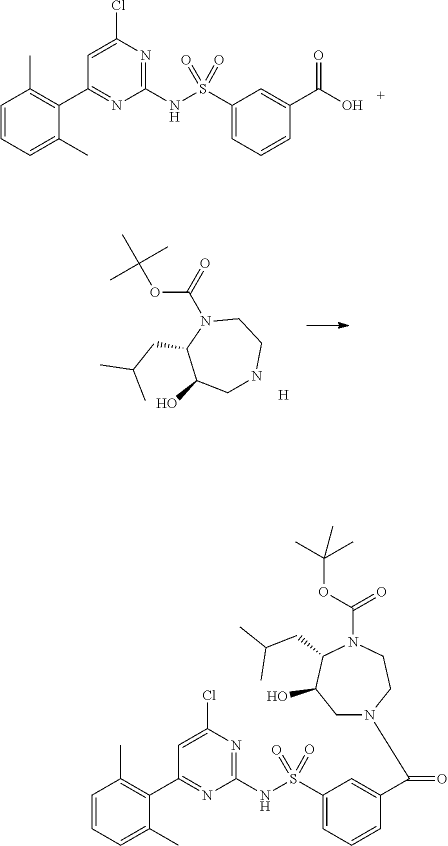

A 100 mL flask was charged under nitrogen with tert-butyl (6R,7S)-6-hydroxy-7-isobutyl-1,4-diazepane-1-carboxylate (1.227 g, 4.460 mmol), anhydrous DMF (23 mL) and 3-[[4-chloro-6-(2,6-dimethylphenyl)pyrimidin-2-yl]sulfamoyl]benzoic acid (1.6 g, 3.829 mmol). After dissolution of the reagents, the mixture was cooled down in an ice bath. DIEA (4.5 mL, 25.84 mmol) and HATU (1.73 g, 4.550 mmol) were added and the mixture was stirred at 0° C. for 4 hours. The reaction was quenched by being poured in citric acid (140 mL of 10% w/v, 72.87 mmol) (10% aqueous) under vigorous stirring and cooled in ice. The resulting white solid was filtered. The wet solid was dissolved in DCM and the solution was dried over sodium sulfate. After concentration, it was purified by flash chromatography on silica gel (120 g column) using a gradient of methanol (0 to 5% over 30 min) in dichloromethane. The product eluted around 3-4% methanol. Evaporation of the solvents gave tert-butyl (6R,7S)-4-[3-[[4-chloro-6-(2,6-dimethylphenyl)pyrimidin-2-yl]sulfamoyl]benzoyl]-6-hydroxy-7-isobutyl-1,4-diazepane-1-carboxylate (1.407 g, 55%) as a pink foamy solid. ESI-MS m/z calc. 671.25446, found 672.33 (M+1)⁺; Retention time: 2.05 minutes; LC method A.

### Step 5: tert-Butyl (16R,17S)-12-(2,6-dimethylphenyl)-17-(2-methylpropyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (Compound 116)

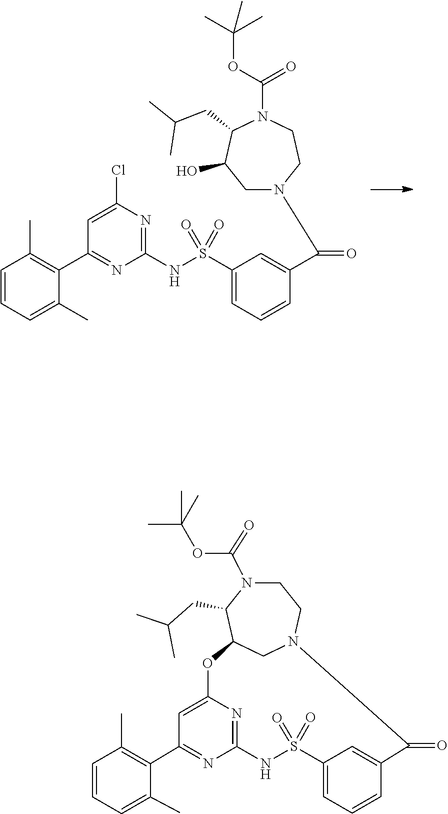 [SMILES: Cc1cccc(C)c1-c1cc(Cl)nc(NS(=O)(=O)c2cccc(C(=O)N3CCN(C(=O)OC(C)(C)C)[C@@H](CC(C)C)[C@H](O)C3)c2)n1 ; Cc1cccc(C)c1-c1cc2nc(n1)NS(=O)(=O)c1cccc(c1)C(=O)N1CCN(C(=O)OC(C)(C)C)[C@@H](CC(C)C)[C@@H](C1)O2]

A 100 mL flask was charged under nitrogen with tert-butyl (6R,7S)-4-[3-[[4-chloro-6-(2,6-dimethylphenyl)pyrimidin-2-yl]sulfamoyl]benzoyl]-6-hydroxy-7-isobutyl-1,4-diazepane-1-carboxylate (1.252 g, 1.862 mmol) and anhydrous DMF (60 mL). The mixture was cooled down in ice. NaH (679 mg of 60% w/w, 16.98 mmol) (60% mineral oil dispersion) was added in small portions. The mixture was stirred under nitrogen at 0° C. for 10 minutes. The ice bath was removed, and the reaction was vigorously stirred under nitrogen for 5 hours. The reaction mixture was slowly poured into an ice-cold citric acid (260 mL of 10% w/v, 135.3 mmol) aqueous solution under stirring. The resulting solid suspension was extracted with EtOAc (3×75 mL). After drying over sodium sulfate, evaporation of the solvents gave a residue that was dissolved in DCM and purified by flash chromatography on silica gel (80 g column) using a gradient of MeOH (0 to 5% over 30 min) in dichloromethane. The product eluted around 3-4% MeOH. Evaporation of the solvents and several cycles of trituration/evaporation in DCM/hexanes gave tert-butyl (16R,17S)-12-(2,6-dimethylphenyl)-17-(2-methylpropyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (442 mg, 36%) as a white solid. ¹H NMR (500 MHz, DMSO-d₆) two conformers visible (70:30) δ 13.35-11.65 (broad m, 1H), 8.61-8.28 (m, 1H), 7.92 (br s, 1H), 7.64 (br s, 2H), 7.25 (t, J=8.3 Hz, 1H), 7.12 (br d, 2H), 6.29 (br s, 1H), 5.9-5.31 (br m, 1H), 4.71-3.99 (m, 2H), 3.93-3.36 (m, 2H), 3.24-2.99 (m, 2H), 2.03 (br s, 6H), 1.90-1.73 (m, 1H), 1.73-1.18 (m, 12H), 1.02-0.73 (m, 6H). ESI-MS m/z calc. 635.2778, found 636.4 (M+1)⁺; Retention time: 1.95 minutes; LC method A.

### Example 57: Preparation of Compound 117

### Step 1: tert-Butyl (6S,7S)-6-hydroxy-7-isobutyl-1,4-diazepane-1-carboxylate

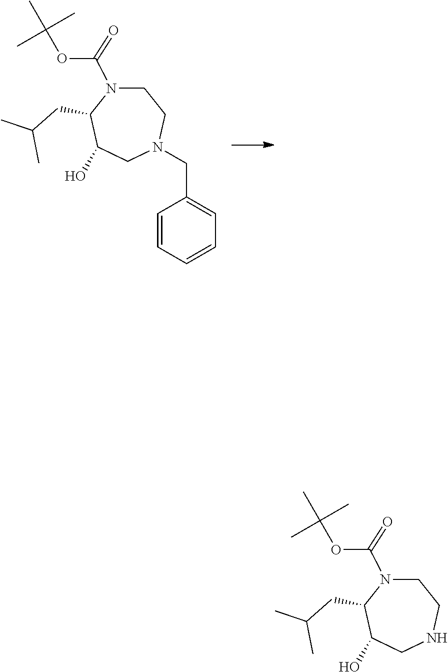 [SMILES: CC(C)C[C@H]1[C@@H](O)CN(Cc2ccccc2)CCN1C(=O)OC(C)(C)C ; CC(C)C[C@H]1[C@@H](O)CNCCN1C(=O)OC(C)(C)C]

A 100 mL round bottom flask was charged with tert-butyl (6S,7S)-4-benzyl-6-hydroxy-7-isobutyl-1,4-diazepane-1-carboxylate (2.43 g, 6.703 mmol) and MeOH (40 mL). The solution was sparged with nitrogen for 10 minutes. Pd(OH)₂ (684 mg of 20% w/w, 0.9742 mmol) (20% wet) was added and the reaction was stirred at room temperature under hydrogen (balloon) for 1.5 days. The solution was sparged with nitrogen for 10 minutes and filtered twice through a pad of Celite. After concentration, the solution was microfiltered through a Whatman 0.45 uM PTFE syringe filter disc and the solvent was evaporated to give tert-butyl (6S,7S)-6-hydroxy-7-isobutyl-1,4-diazepane-1-carboxylate (1.788 g, 98%) as a pale brown resin. ESI-MS m/z calc. 272.21, found 273.16 (M+1)⁺; Retention time: 1.06 minutes; LC method A.

### Step 2: tert-Butyl (6S,7S)-4-[3-[[4-chloro-6-(2,6-dimethylphenyl)pyrimidin-2-yl]sulfamoyl]benzoyl]-6-hydroxy-7-isobutyl-1,4-diazepane-1-carboxylate

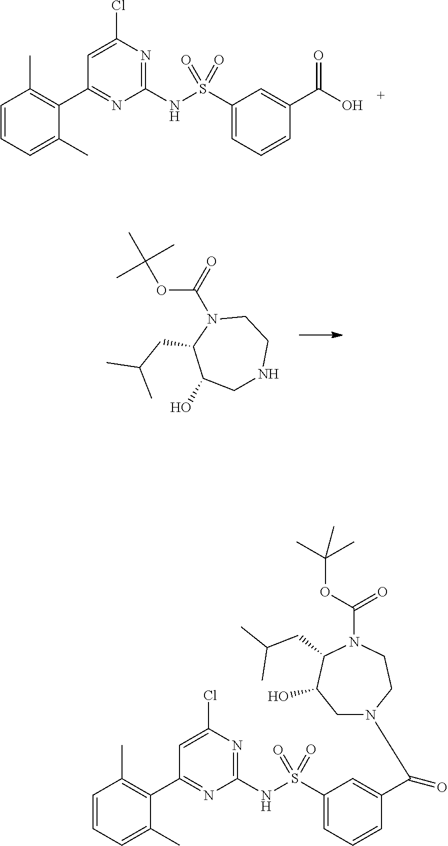 [SMILES: Cc1cccc(C)c1-c1cc(Cl)nc(NS(=O)(=O)c2cccc(C(=O)O)c2)n1 ; CC(C)C[C@H]1[C@@H](O)CNCCN1C(=O)OC(C)(C)C ; Cc1cccc(C)c1-c1cc(Cl)nc(NS(=O)(=O)c2cccc(C(=O)N3CCN(C(=O)OC(C)(C)C)[C@@H](CC(C)C)[C@@H](O)C3)c2)n1]

A 100 mL flask was charged under nitrogen with tert-butyl (6S,7S)-6-hydroxy-7-isobutyl-1,4-diazepane-1-carboxylate (1.788 g, 6.564 mmol), anhydrous DMF (30 mL) and 3-[[4-chloro-6-(2,6-dimethylphenyl)pyrimidin-2-yl]sulfamoyl]benzoic acid (2.2 g, 5.265 mmol). After dissolution of the reagents, the mixture was cooled down in an ice bath. DIEA (6.2 mL, 35.59 mmol) and HATU (2.56 g, 6.733 mmol) were added and the mixture was stirred at 0° C. for 20 min. The reaction was quenched by being poured in citric acid (190 mL of 10% w/v, 98.89 mmol)(10% aqueous) under vigorous stirring and cooled in ice. The resulting white solid was filtered and sucked dry. The solid was dissolved in DCM. After concentration, it was purified by flash chromatography on silica gel (220 g column) using a gradient of methanol (0 to 5% over 30 min) in dichloromethane. The product eluted around 2-3% methanol. Evaporation of the solvents gave tert-butyl (6S,7S)-4-[3-[[4-chloro-6-(2,6-dimethylphenyl)pyrimidin-2-yl]sulfamoyl]benzoyl]-6-hydroxy-7-isobutyl-1,4-diazepane-1-carboxylate (2.39 g, 68%) as a white foamy solid. ESI-MS m/z calc. 671.25446, found 672.44 (M+1)⁺; Retention time: 2.07 minutes; LC method A.

### Step 3: tert-Butyl (16S,17S)-12-(2,6-dimethylphenyl)-17-(2-methylpropyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (Compound 117)

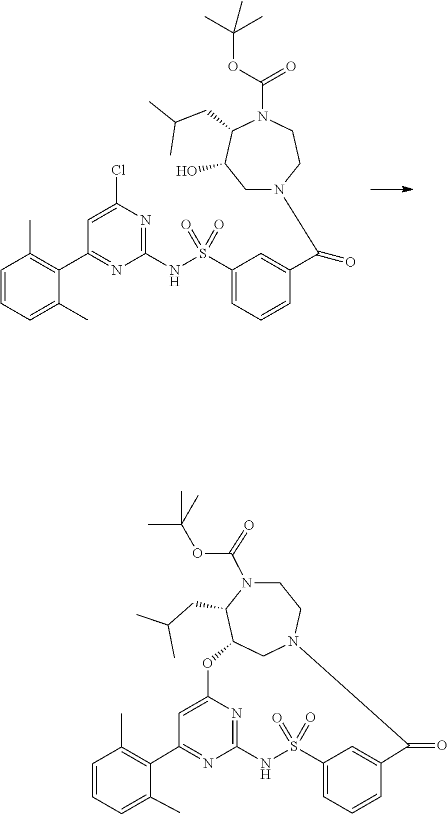 [SMILES: Cc1cccc(C)c1-c1cc(Cl)nc(NS(=O)(=O)c2cccc(C(=O)N3CCN(C(=O)OC(C)(C)C)[C@@H](CC(C)C)[C@@H](O)C3)c2)n1 ; Cc1cccc(C)c1-c1cc2nc(n1)NS(=O)(=O)c1cccc(c1)C(=O)N1CCN(C(=O)OC(C)(C)C)[C@@H](CC(C)C)[C@H](C1)O2]

A 100 mL flask was charged under nitrogen with tert-butyl (6S,7S)-4-[3-[[4-chloro-6-(2,6-dimethylphenyl)pyrimidin-2-yl]sulfamoyl]benzoyl]-6-hydroxy-7-isobutyl-1,4-diazepane-1-carboxylate (2.39 g, 3.555 mmol) and anhydrous DMF (100 mL). The mixture was cooled down in ice. NaH (1.14 g of 60% w/w, 28.50 mmol) (60% mineral oil dispersion) was added in two equal portions, added 3 minutes after each other. The mixture was stirred under nitrogen at 0° C. for 10 minutes. The ice bath was removed, and the reaction was vigorously stirred under nitrogen for 2 hours. The reaction mixture was slowly poured into an ice-cold citric acid (500 mL of 10% w/v, 260.2 mmol) aqueous solution under stirring. The resulting solid suspension was extracted with EtOAc (3×120 mL). After drying over sodium sulfate, evaporation of the solvents gave a residue that was dissolved in DCM and purified by flash chromatography on silica gel (120 g column) using a gradient of MeOH (0 to 5% over 30 min) in dichloromethane. The product eluted around 3-4% MeOH. Evaporation of the solvents and several cycles of trituration/evaporation in DCM/hexanes gave tert-butyl (16S,17S)-12-(2,6-dimethylphenyl)-17-(2-methylpropyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (749 mg, 33%) as a white solid. ESI-MS m/z calc. 635.2778, found 636.4 (M+1)⁺; Retention time: 1.92 minutes (LC method A). This material (91% pure) was used for the next step without any further purification.

A small amount of material (45 mg) was dissolved in DMSO (1 mL) and was purified by reverse phase preparative HPLC (Cis) using a gradient of acetonitrile in water (1 to 99% over 15 min) and HCl as a modifier to give tert-butyl (16S,17S)-12-(2,6-dimethylphenyl)-17-(2-methylpropyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (24 mg, 52%) as white solid. ¹H NMR (499 MHz, DMSO-d₆) two rotamers visible ratio (60:40). δ 13.22-12.00 (broad m, 1H), 8.39-8.21 (m, 1H), 7.89 (br s, 1H), 7.65 (br s, 2H), 7.25 (d, J=8.3 Hz, 1H), 7.12 (d, J=7.6 Hz, 2H), 6.31 (br s, 1H), 5.68-5.45 (m, 1H), 4.71-4.33 (m, 2H), 4.08-3.83 (m, 1H), 3.44 (q, J=13.2 Hz, 1H), 3.30-3.18 (m, 2H overlapped with water signal), 3.14-3.02 (m, 1H), 2.15-1.87 (m, 7H), 1.56 (d, J=10.3 Hz, 2H), 1.50-1.37 (m, 9H), 1.02-0.78 (m, 6H). ESI-MS m/z calc. 635.2778, found 636.4 (M+1)⁺; Retention time: 1.94 minutes; LC method A.

### Example 58: Preparation of Compound 118

### Step 1: (16S,17R)-12-(2,6-Dimethylphenyl)-17-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 118)

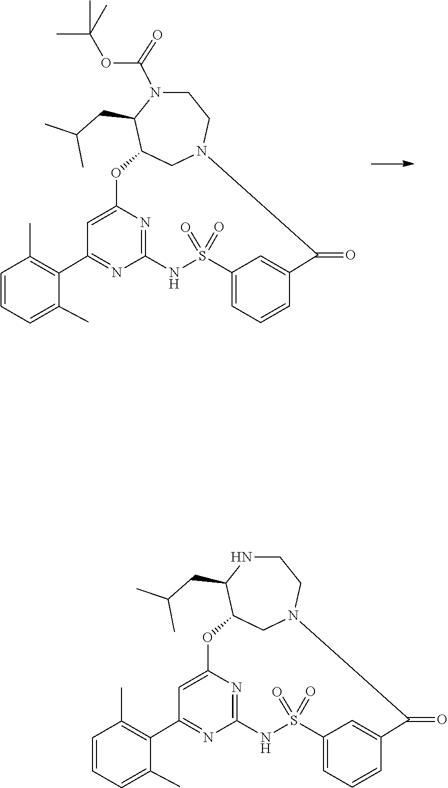 [SMILES: Cc1cccc(C)c1-c1cc2nc(n1)NS(=O)(=O)c1cccc(c1)C(=O)N1CCN(C(=O)OC(C)(C)C)[C@H](CC(C)C)[C@H](C1)O2 ; Cc1cccc(C)c1-c1cc2nc(n1)NS(=O)(=O)c1cccc(c1)C(=O)N1CCN[C@H](CC(C)C)[C@H](C1)O2]

A 100 mL flask containing tert-butyl (16S,17R)-12-(2,6-dimethylphenyl)-17-(2-methylpropyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (303 mg, 0.4671 mmol) was charged under nitrogen with DCM (5 mL). HCl (5 mL of 4 M, 20.00 mmol) (4 M dioxane solution) was added and the mixture was stirred at room temperature for 2.5 hours. The volatiles were removed by evaporation and the residue was triturated in DCM/hexanes and the solvents were evaporated. The operation was repeated until a solid was obtained. Drying under vacuum gave (16S,17R)-12-(2,6-dimethylphenyl)-17-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (306 mg, 103%) as an off-white solid. ESI-MS m/z calc. 535.22534, found 536.49 (M+1)⁺; Retention time: 1.04 minutes (LC method A). This material was used for the next step without any further purification.

A small amount of material (20 mg) was purified by reverse phase preparative HPLC (Cis) using a gradient of acetonitrile in water (1 to 99% over 15 min) and HCl as a modifier to give (16S,17R)-12-(2,6-dimethylphenyl)-17-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (11 mg, 54%) as an off-white solid. ESI-MS m/z calc. 535.22534, found 536.5 (M+1)⁺; Retention time: 1.05 minutes; LC method A.

### Example 59: Preparation of Compound 119 and Compound 120

### Step 1: (16S,17R)-18-(4,4-Difluorocyclohexyl)-12-(2,6-dimethylphenyl)-17-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione, (16S,17R)-12-(2,6-dimethylphenyl)-17-(2-methylpropyl)-18-{spiro[3.4]octan-2-yl}-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 119), and (16S,17R)-12-(2,6-dimethylphenyl)-17-(2-methylpropyl)-18-{spiro[3.5]nonan-2-yl}-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 120)

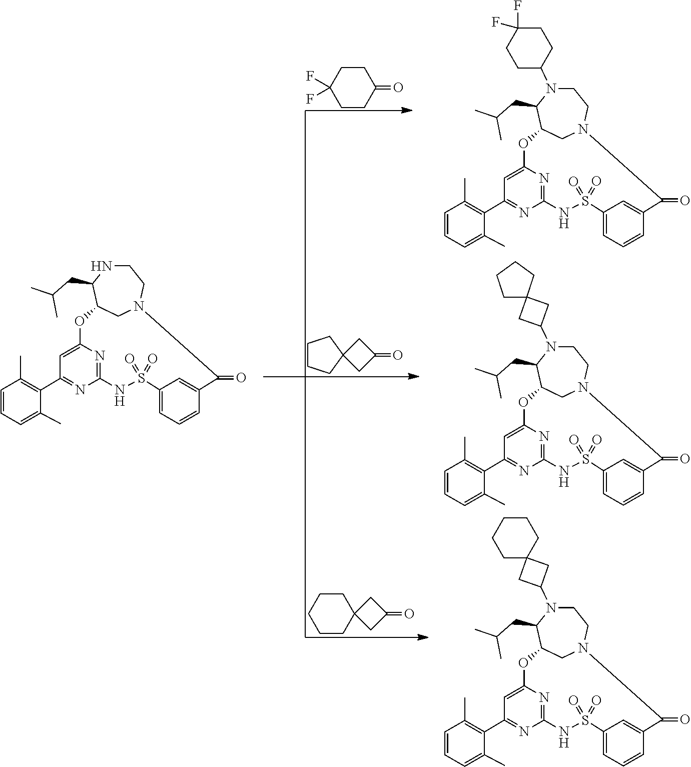

For each of the three reactions, a 4 mL vial was separately charged with (16S,17R)-12-(2,6-dimethylphenyl)-17-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (20 mg, 0.03146 mmol), anhydrous DCM (300 μL), DIEA (10 μL, 0.05741 mmol), acetic acid (13 μL, 0.2286 mmol) and the corresponding ketones 4,4-difluorocyclohexanone (25 mg, 0.1864 mmol) (reaction A), spiro[3.4]octan-2-one (25 mg, 0.2013 mmol) (reaction B) and spiro[3.5]nonan-2-one (25 mg, 0.1809 mmol) (reaction C). The vial was capped and stirred at room temperature for 20 minutes. Sodium triacetoxyborohydride (25 mg, 0.1180 mmol) was added. The vial was capped and stirred at room temperature for 16 hours (reaction B and C). For reaction A, another amount of reagent 4,4-difluorocyclohexanone (136 mg) and sodium triacetoxyborohydride (25 mg, 0.1180 mmol) were added and the mixture was stirred for 4 hours. Methanol (100 μL) was added. DCM was evaporated and the residue was taken in DMSO (1 mL). The solution was microfiltered (0.45 μM) and purified by reverse phase preparative HPLC (Cis) using a gradient of acetonitrile in water (1 to 99% over 15 min) and HCl as a modifier (two purifications per compound for B and C). The compound from reaction A was purified a third time using ammonium formate as a modifier. Evaporation gave the three following products as off-a white solid:

(16S,17R)-18-(4,4-Difluorocyclohexyl)-12-(2,6-dimethylphenyl)-17-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (2 mg, 10%). ESI-MS m/z calc. 653.2847, found 654.55 (M+1)⁺; Retention time: 1.6 minutes (LC method A).

(16S,17R)-12-(2,6-Dimethylphenyl)-17-(2-methylpropyl)-18-{spiro[3.4]octan-2-yl}-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (7.5 mg, 34%). ESI-MS m/z calc. 643.3192, found 644.43 (M+1)⁺; Retention time: 1.55 minutes (LC method A).

(16S,17R)-12-(2,6-Dimethylphenyl)-17-(2-methylpropyl)-18-{spiro[3.5]nonan-2-yl}-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (5.7 mg, 26%). ESI-MS m/z calc. 657.3349, found 658.4 (M+1)⁺; Retention time: 1.63 minutes (LC method A).

### Example 60: Preparation of Compound 121

### Step 1: (16S,17R)-12-(2,6-Dimethylphenyl)-18-methyl-17-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 121)

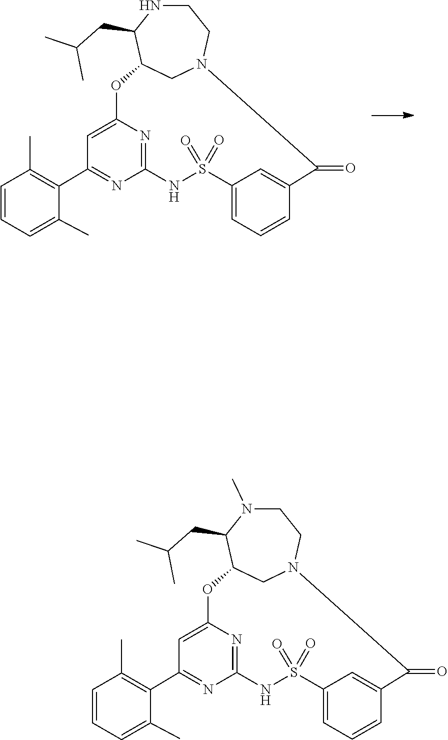 [SMILES: Cc1cccc(C)c1-c1cc2nc(n1)NS(=O)(=O)c1cccc(c1)C(=O)N1CCN[C@H](CC(C)C)[C@H](C1)O2 ; Cc1cccc(C)c1-c1cc2nc(n1)NS(=O)(=O)c1cccc(c1)C(=O)N1CCN(C)[C@H](CC(C)C)[C@H](C1)O2]

(16S,17R)-12-(2,6-Dimethylphenyl)-17-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (20 mg, 0.03146 mmol) was dissolved in formic acid (250 μL) (88% aqueous) and combined with aqueous formaldehyde (900 μL, 32.67 mmol) (37% aqueous) and heated to 90° C. for 2 hours in a screwcap vial. The reaction mixture was then partially concentrated by blowing nitrogen and diluted with methanol. The solution was microfiltered through a syringe filter disc and purified by reverse phase preparative HPLC (Cis) using a gradient of acetonitrile in water (1 to 99% over 15 min) and HCl as a modifier to give (16S,17R)-12-(2,6-dimethylphenyl)-18-methyl-17-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (5.6 mg, 30%) as a white solid. ESI-MS m/z calc. 549.24097, found 550.46 (M+1)⁺; Retention time: 1.04 minutes; LC method A.

### Example 61: Preparation of Compound 122

### Step 1: (16S,17R)-18-(3,3-Dimethylbutanoyl)-12-(2,6-dimethylphenyl)-17-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 122)

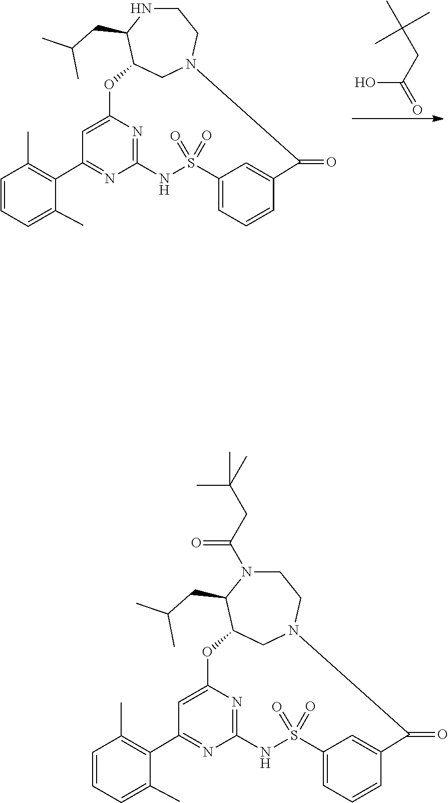

A 4 mL vial was charged with (16S,17R)-12-(2,6-dimethylphenyl)-17-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (20 mg, 0.03146 mmol), 3,3-dimethylbutanoic acid (10 μL, 0.07851 mmol), DMF (200 μL), DIEA (27 μL, 0.1550 mmol) and HATU (30 mg, 0.07890 mmol). The vial was capped, and the mixture was stirred at room temperature for 2 hours. LCMS showed 30% conversion. Another amount of 3,3-dimethylbutanoic acid (50 μL, 0.3926 mmol) was added and the mixture was stirred overnight at room temperature. After 16 hours, the mixture was diluted with DMSO (800 μL). The solution was microfiltered through a syringe filter disc and purified by reverse phase preparative HPLC (Cis) using a gradient of acetonitrile in water (1 to 99% over 15 min) and HCl as a modifier to give (16S,17R)-18-(3,3-dimethylbutanoyl)-12-(2,6-dimethylphenyl)-17-(2-methylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (10.5 mg, 52%) as a white solid. ESI-MS m/z calc. 633.29846, found 634.42 (M+1)⁺; Retention time: 1.89 minutes; LC method A.

### Example 62: Preparation of Compound 123 and Compound 124

### Step 1: (3R,6R)-4-Benzyl-6-hydroxy-3-isopropyl-1,4-diazepan-2-one

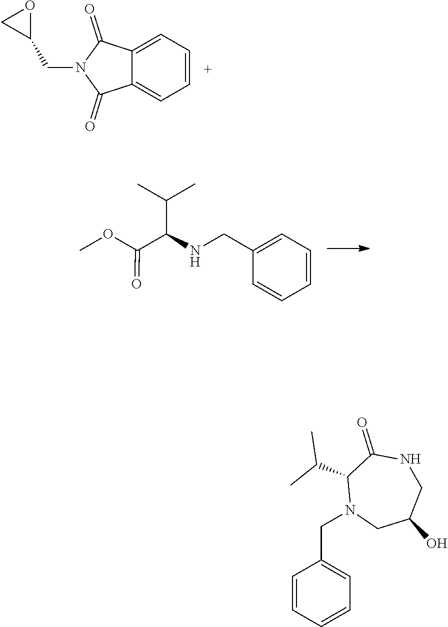 [SMILES: O=C1c2ccccc2C(=O)N1C[C@@H]1CO1 ; COC(=O)[C@H](NCc1ccccc1)C(C)C ; CC(C)[C@@H]1C(=O)NC[C@@H](O)CN1Cc1ccccc1]

To a solution of 2-[[(2R)-oxiran-2-yl]methyl]isoindoline-1,3-dione (7.3 g, 35.926 mmol) in ACN (50 mL) was added methyl (2R)-2-(benzylamino)-3-methyl-butanoate (7.98 g, 36.060 mmol) and magnesium perchlorate (12.1 g, 54.210 mmol). The reaction mixture was stirred at room temperature overnight before being diluted with water (70 mL) and extracted with DCM (3×75 mL). The combined organic layers were washed with brine (70 mL), dried over anhydrous sodium sulfate and concentrated under vacuum. The organic residue was dissolved in methanol (160 mL) and hydrazine hydrate (3.6056 g, 4.8 mL, 52.578 mmol) was added to the reaction. The reaction was stirred at 65° C. for 24 hours. The reaction was cooled to room temperature and the white solid was filtered off. The filtrate was concentrated and then diluted with 1 N NaOH (200 mL) before being extracted with ethyl acetate (3×200 mL). The combined organic layers were washed with brine (200 mL), dried over sodium sulfate and concentrated before being purified by silica gel chromatography eluting 0-5% DCM-MeOH to give (3R,6R)-4-benzyl-6-hydroxy-3-isopropyl-1,4-diazepan-2-one (3.21 g, 34%). ¹H NMR (250 MHz, CDCl₃) δ 7.40-7.15 (m, 5H), 5.75 (s, 1H), 4.13-3.76 (m, 2H), 3.65-3.59 (m, 1H), 3.45-3.30 (m, 2H), 3.28-3.01 (m, 2H), 2.88-2.54 (m, 1H), 2.43-2.12 (m, 1H), 1.19-0.67 (m, 6H). ESI-MS m/z calc. 262.16812, found 263.2 (M+1)⁺; Retention time: 1.94 minutes; LC method T.

### Step 2: (2R,6R)-1-Benzyl-2-isopropyl-1,4-diazepan-6-ol

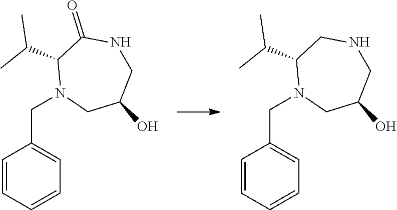 [SMILES: CC(C)[C@@H]1C(=O)NC[C@@H](O)CN1Cc1ccccc1 ; CC(C)[C@@H]1CNC[C@@H](O)CN1Cc1ccccc1]

Into a solution of (3R,6R)-4-benzyl-6-hydroxy-3-isopropyl-1,4-diazepan-2-one (3.21 g, 12.236 mmol) in THE (100 mL) was added LAH (5.1 g, 134.37 mmol). The reaction was stirred at 40° C. for 3 days before being cooled to room temperature. The reaction was quenched with water (5.1 mL), 15% NaOH (aqueous) (5.1 mL) and water (15.3 mL) at 0° C. subsequently. The reaction mixture was stirred for another 30 minutes, then it was filtered through a pad of Celite. The filter cake was washed with THE (3×50 mL). The combined filtrate was concentrated under vacuum to give (2R,6R)-1-benzyl-2-isopropyl-1,4-diazepan-6-ol (3.23 g, 106%). ESI-MS m/z calc. 248.18886, found 249.3 (M+1)⁺; Retention time: 1.46 minutes; LC method T.

### Step 3: tert-Butyl (3R,6S)-4-benzyl-6-hydroxy-3-isopropyl-1,4-diazepane-1-carboxylate

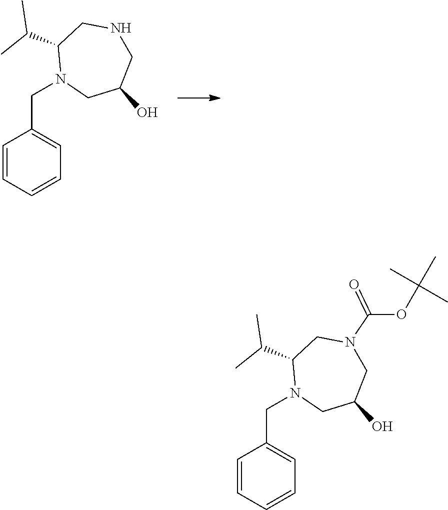 [SMILES: CC(C)[C@@H]1CNC[C@@H](O)CN1Cc1ccccc1 ; CC(C)[C@@H]1CN(C(=O)OC(C)(C)C)C[C@@H](O)CN1Cc1ccccc1]

To a solution of (2R,6R)-1-benzyl-2-isopropyl-1,4-diazepan-6-ol (3.23 g, 13.005 mmol) in DCM (40 mL) was added Boc anhydride (4.26 g, 19.519 mmol) and triethylamine (1.9602 g, 2.7 mL, 19.371 mmol) at room temperature and stirred overnight. The reaction was quenched with brine (50 mL). The two layers were separated, and the aqueous layer was extracted with DCM (2×50 mL). The combined organic layers were dried over anhydrous magnesium sulfate and concentrated under vacuum. The residue was purified by silica gel chromatography using 0-5% DCM-MeOH to give tert-butyl (3R,6S)-4-benzyl-6-hydroxy-3-isopropyl-1,4-diazepane-1-carboxylate (2.61 g, 58%). ¹H NMR (250 MHz, CDCl₃) δ 7.29 (dd, J=8.9, 6.0 Hz, 5H), 4.07-3.82 (m, 2H), 3.78-3.56 (m, 2H), 3.52 (dd, J=10.9, 4.5 Hz, 2H), 3.40-3.14 (m, 2H), 2.95-2.68 (m, 1H), 2.61-2.36 (m, 1H), 1.88 (d, J=6.9 Hz, 1H), 1.50 (d, J=7.5 Hz, 9H), 1.18-0.68 (m, 6H). ESI-MS m/z calc. 348.2413, found 349.2 (M+1)⁺; Retention time: 2.28 minutes; LC method T.

### Step 4: tert-Butyl (3R,6S)-6-hydroxy-3-isopropyl-1,4-diazepane-1-carboxylate

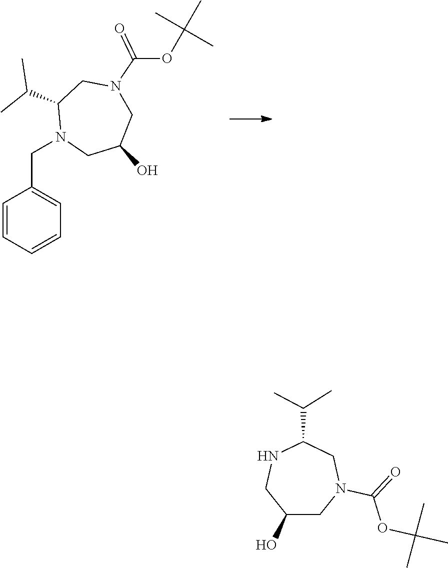 [SMILES: CC(C)[C@@H]1CN(C(=O)OC(C)(C)C)C[C@@H](O)CN1Cc1ccccc1 ; CC(C)[C@@H]1CN(C(=O)OC(C)(C)C)C[C@@H](O)CN1]

To a solution of tert-butyl (3R,6S)-4-benzyl-6-hydroxy-3-isopropyl-1,4-diazepane-1-carboxylate (2.61 g, 7.4897 mmol) in methanol (52 mL) was added Palladium (800 mg, 0.7517 mmol) on carbon and ammonium formate (1.4 g, 22.203 mmol). The reaction was stirred at 65° C. for 2 hour. Palladium was removed by filtration, and the solution was concentrated under vacuum. The residue was diluted with DCM (100 mL), and washed with water (50 mL) and brine (50 mL), dried over anhydrous sodium sulfate and concentrated under vacuum to give tert-butyl (3R,6S)-6-hydroxy-3-isopropyl-1,4-diazepane-1-carboxylate (1.82 g, 87%). ¹H NMR (500 MHz, DMSO-d₆) δ 4.62 (dd, J=12.9, 5.1 Hz, 1H), 3.60-3.46 (m, 1H), 3.44-3.35 (m, 2H), 3.34-3.13 (m, 2H), 3.10-2.95 (m, 2H), 2.90 (dd, J=13.7, 9.7 Hz, 1H), 2.43-2.33 (m, 1H), 2.27 (ddd, J=16.7, 13.3, 8.9 Hz, 1H), 1.77 (s, 1H), 1.58-1.49 (m, 1H), 1.38 (s, 9H), 0.90-0.81 (m, 6H). ESI-MS m/z calc. 258.19434, found 259.2 (M+1)⁺; Retention time: 1.36 minutes; LC method T.

### Step 5: tert-Butyl (3R,6S)-4-[3-[[4-chloro-6-(2,6-dimethylphenyl)pyrimidin-2-yl]sulfamoyl]benzoyl]-6-hydroxy-3-isopropyl-1,4-diazepane-1-carboxylate

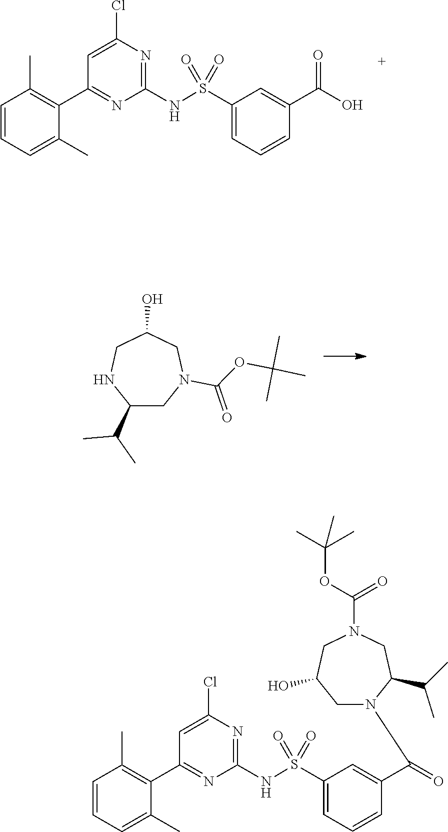 [SMILES: Cc1cccc(C)c1-c1cc(Cl)nc(NS(=O)(=O)c2cccc(C(=O)O)c2)n1 ; CC(C)[C@@H]1CN(C(=O)OC(C)(C)C)C[C@@H](O)CN1 ; Cc1cccc(C)c1-c1cc(Cl)nc(NS(=O)(=O)c2cccc(C(=O)N3C[C@H](O)CN(C(=O)OC(C)(C)C)C[C@H]3C(C)C)c2)n1]

A 100 mL flask was charged under nitrogen with tert-butyl (3R,6S)-6-hydroxy-3-(1-methylethyl)-1,4-diazepane-1-carboxylate (1.753 g, 6.785 mmol), anhydrous DMF (35 mL) and 3-[[4-chloro-6-(2,6-dimethylphenyl)pyrimidin-2-yl]sulfamoyl]benzoic acid (2.39 g, 5.720 mmol). After dissolution of the reagents, the mixture was cooled down in an ice bath. DIEA (6.7 mL, 38.47 mmol) and HATU (2.61 g, 6.864 mmol) were added and the mixture was stirred at 0° C. for 25 min, then at room temperature for 6 hours. The reaction was quenched by being poured in citric acid (200 mL of 10% w/v, 104.1 mmol)(10% aqueous) under vigorous stirring and cooled in ice. The resulting off-white solid was filtered. The wet solid was dissolved in DCM and the solution was dried over sodium sulfate. After concentration, it was purified by flash chromatography on silica gel (120 g column) using a gradient of methanol (0 to 5% over 30 min) in dichloromethane. The product eluted around 3-4% methanol. Evaporation of the solvents gave tert-butyl (3R,6S)-4-[3-[[4-chloro-6-(2,6-dimethylphenyl)pyrimidin-2-yl]sulfamoyl]benzoyl]-6-hydroxy-3-isopropyl-1,4-diazepane-1-carboxylate (1.607 g, 43%) as an off-white foamy solid. ESI-MS m/z calc. 657.2388, found 658.36 (M+1)⁺; Retention time: 1.96 minutes; LC method A.

### Step 6: tert-Butyl (16S,20R)-12-(2,6-dimethylphenyl)-2,8,8-trioxo-20-(propan-2-yl)-15-oxa-8λ⁶-thia-1,9,11,18,22-pentaazatetracyclo[14.4.1.13,7.110,14]tricosa-3(23),4,6,10(22),11,13-hexaene-18-carboxylate (Compound 124)

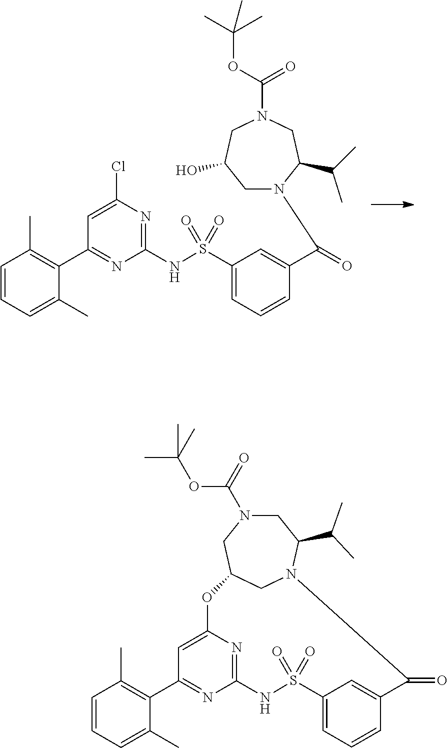 [SMILES: Cc1cccc(C)c1-c1cc(Cl)nc(NS(=O)(=O)c2cccc(C(=O)N3C[C@H](O)CN(C(=O)OC(C)(C)C)C[C@H]3C(C)C)c2)n1 ; Cc1cccc(C)c1-c1cc2nc(n1)NS(=O)(=O)c1cccc(c1)C(=O)N1C[C@@H](CN(C(=O)OC(C)(C)C)C[C@H]1C(C)C)O2]

A 250 mL flask was charged under nitrogen with tert-butyl (3R,6S)-4-[3-[[4-chloro-6-(2,6-dimethylphenyl)pyrimidin-2-yl]sulfamoyl]benzoyl]-6-hydroxy-3-isopropyl-1,4-diazepane-1-carboxylate (1.607 g, 2.441 mmol) and anhydrous DMF (80 mL). The mixture was cooled down in ice. NaH (780 mg of 60% w/w, 19.50 mmol) (60% mineral oil dispersion) was added in two equal portions, added 3 minutes after each other. The mixture was stirred under nitrogen at 0° C. for 10 minutes. The ice bath was removed, and the reaction was vigorously stirred under nitrogen for 3 hours. The reaction mixture was slowly poured into an ice-cold citric acid (300 mL of 10% w/v, 156.1 mmol) aqueous solution under stirring. The resulting solid suspension was extracted with EtOAc (3×75 mL). After drying over sodium sulfate, evaporation of the solvents gave a residue that was dissolved in DCM and purified by flash chromatography on silica gel (120 g column) using a gradient of MeOH (0 to 5% over 30 min) in dichloromethane. The product eluted around 3-4% MeOH. Evaporation of the solvents and several cycles of trituration/evaporation in DCM/hexanes gave tert-butyl (16S,20R)-12-(2,6-dimethylphenyl)-2,8,8-trioxo-20-(propan-2-yl)-15-oxa-8λ⁶-thia-1,9,11,18,22-pentaazatetracyclo[14.4.1.13,7.110,14]tricosa-3(23),4,6,10(22),11,13-hexaene-18-carboxylate (539 mg, 35%) as a white solid. ESI-MS m/z calc. 621.2621, found 622.32 (M+1)⁺; Retention time: 1.83 minutes; LC method A.

### Step 7: (16R,20R)-12-(2,6-Dimethylphenyl)-20-(propan-2-yl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 123)

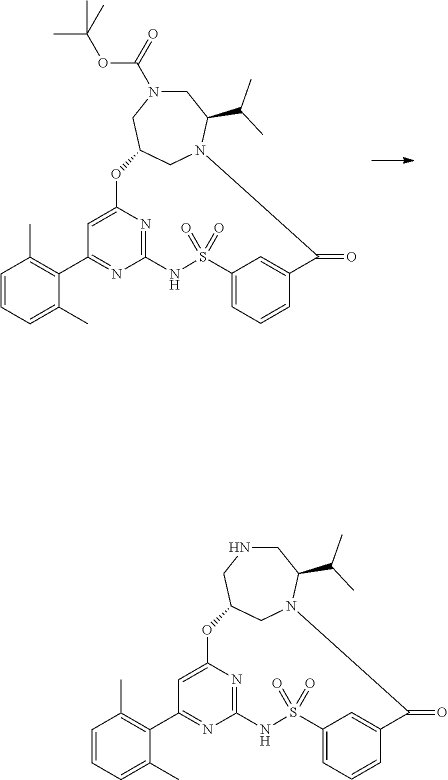 [SMILES: Cc1cccc(C)c1-c1cc2nc(n1)NS(=O)(=O)c1cccc(c1)C(=O)N1C[C@@H](CN(C(=O)OC(C)(C)C)C[C@H]1C(C)C)O2 ; Cc1cccc(C)c1-c1cc2nc(n1)NS(=O)(=O)c1cccc(c1)C(=O)N1C[C@@H](CNC[C@H]1C(C)C)O2]

A 100 mL flask containing tert-butyl (16S,20R)-12-(2,6-dimethylphenyl)-2,8,8-trioxo-20-(propan-2-yl)-15-oxa-8λ⁶-thia-1,9,11,18,22-pentaazatetracyclo[14.4.1.13,7.110,14]tricosa-3(23),4,6,10(22),11,13-hexaene-18-carboxylate (527 mg, 0.8307 mmol) was charged under nitrogen with DCM (5 mL). HCl (4 mL of 4 M, 16.00 mmol) (4 M dioxane solution) was added and the mixture was stirred at room temperature for 8 hours. The volatiles were removed by evaporation and the residue was triturated in DCM/hexanes and the solvents were evaporated. The operation was repeated until a solid was obtained. Drying under vacuum gave (16R,20R)-12-(2,6-dimethylphenyl)-20-(propan-2-yl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (491 mg, 102%) as a white solid. ¹H NMR (500 MHz, DMSO-d₆+10% D₂O) δ 8.85 (s, 1H), 7.89 (d, J=7.9 Hz, 1H), 7.67 (t, J=7.8 Hz, 1H), 7.51 (d, J 7.6 Hz, 1H), 7.26 (t, J=7.6 Hz, 1H), 7.12 (d, J=7.7 Hz, 2H), 6.30 (s, 1H), 5.67-5.55 (m, 1H), 3.85 (t, J=12.4 Hz, 1H), 3.59-3.40 (m, 5H), 3.01 (dd, J=13.9, 10.9 Hz, 1H), 2.92-2.80 (m, 1H), 2.03 (broad s, 6H), 1.04-0.89 (m, 6H). ESI-MS m/z calc. 521.20966, found 522.41 (M+1)⁺; Retention time: 1.06 minutes; LC method A.

### Example 63: Preparation of Compound 125

### Step 8: (16R,20R)-12-(2,6-Dimethylphenyl)-18-methyl-20-(propan-2-yl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 125)

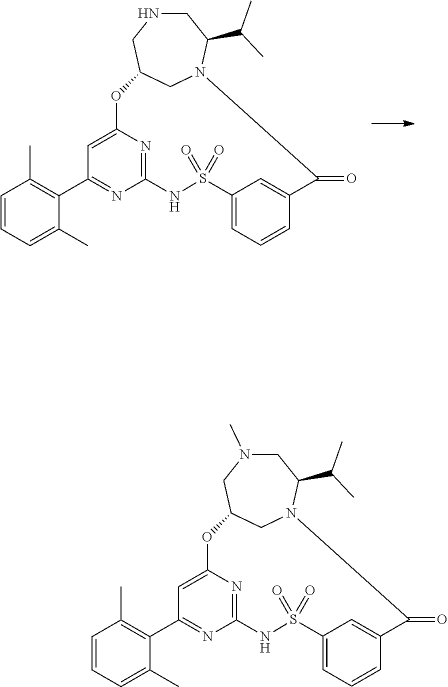 [SMILES: Cc1cccc(C)c1-c1cc2nc(n1)NS(=O)(=O)c1cccc(c1)C(=O)N1C[C@@H](CNC[C@H]1C(C)C)O2 ; Cc1cccc(C)c1-c1cc2nc(n1)NS(=O)(=O)c1cccc(c1)C(=O)N1C[C@@H](CN(C)C[C@H]1C(C)C)O2]

(16R,20R)-12-(2,6-dimethylphenyl)-20-(propan-2-yl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (28 mg, 0.04816 mmol) was dissolved in formic acid (250 μL) (88% aqueous) and combined with aqueous formaldehyde (900 μL, 32.67 mmol) (37% aqueous) and heated to 90° C. for 15 hours in a screwcap vial. The reaction mixture was then partially concentrated by blowing nitrogen, diluted with methanol. The solution was microfiltered through a PTFE syringe filter disc and purified by reverse phase preparative HPLC (Cis) using a gradient of acetonitrile in water (1 to 99% over 15 min) and HCl as a modifier to give (16R,20R)-12-(2,6-dimethylphenyl)-18-methyl-20-(propan-2-yl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (18.1 mg, 64%) as a white solid. ESI-MS m/z calc. 535.22534, found 536.6 (M+1)⁺; Retention time: 1.06 minutes; LC method A.

### Example 64: Preparation of Compound 126, Compound 127, and Compound 128

### Step 8: (16R,20R)-18-(4,4-Difluorocyclohexyl)-12-(2,6-dimethylphenyl)-20-(propan-2-yl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 126), and (16R,20R)-12-(2,6-dimethylphenyl)-20-(propan-2-yl)-18-{spiro[3.4]octan-2-yl}-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 127), and (16R,20R)-12-(2,6-dimethylphenyl)-20-(propan-2-yl)-18-{spiro[3.5]nonan-2-yl}-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 128)

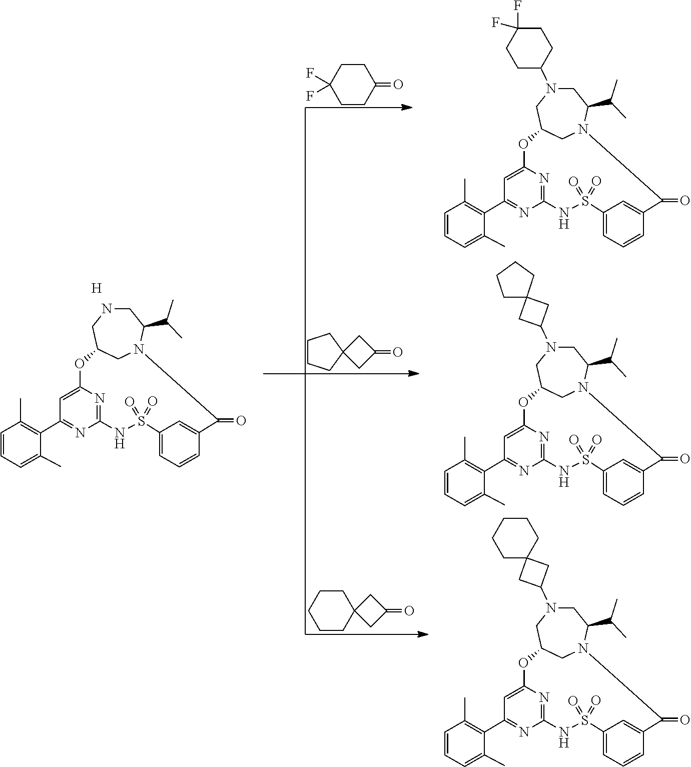

For each of the three reactions, a 4 mL vial was separately charged with (16R,20R)-12-(2,6-dimethylphenyl)-20-(propan-2-yl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (20 mg, 0.03440 mmol), anhydrous DCM (300 μL), DIEA (10 μL, 0.05741 mmol), acetic acid (13 μL, 0.2286 mmol) and the corresponding ketones 4,4-difluorocyclohexanone (25 mg, 0.1864 mmol) (reaction A), spiro[3.4]octan-2-one (25 mg, 0.2013 mmol) (reaction B) and spiro[3.5]nonan-2-one (25 mg, 0.1809 mmol) (reaction C). The vial was capped and stirred at room temperature for 20 minutes. Sodium triacetoxyborohydride (25 mg, 0.1180 mmol) was added. The vial was capped and stirred at room temperature for 17 hours. Methanol (100 μL) was added. DCM was evaporated and the residue was taken in DMSO (1 mL). The solution was microfiltered (0.45 μM) and purified by reverse phase preparative HPLC (Cis) using a gradient of acetonitrile in water (1 to 99% over 15 min) and HCl as a modifier. Geneva evaporation gave the 3 following products as a white solid:

(16R,20R)-18-(4,4-difluorocyclohexyl)-12-(2,6-dimethylphenyl)-20-(propan-2-yl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (12.5 mg, 52%). ESI-MS m/z calc. 639.2691, found 640.4 (M+1)⁺; Retention time: 1.4 minutes (LC method A).

(16R,20R)-12-(2,6-dimethylphenyl)-20-(propan-2-yl)-18-{spiro[3.4]octan-2-yl}-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (13.2 mg, 55%). ESI-MS m/z calc. 629.3036, found 630.61 (M+1)⁺; Retention time: 1.4 minutes (LC method A).

(16R,20R)-12-(2,6-dimethylphenyl)-20-(propan-2-yl)-18-{spiro[3.5]nonan-2-yl}-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (16.1 mg, 66%). ESI-MS m/z calc. 643.3192, found 644.43 (M+1)⁺; Retention time: 1.44 minutes (LC method A).

### Example 65: Preparation of Compound 129, Compound 130, and Compound 131

### Step 1: Methyl (2S)-2-amino-5-methyl-hexanoate

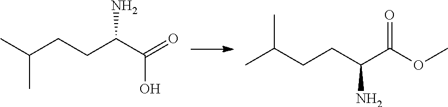

(2S)-2-amino-5-methyl-hexanoic acid (10.02 g, 69.009 mmol) was dissolved in MeOH (510 mL) and SOCl2 (8.4 g, 5.1502 mL, 70.605 mmol) was added slowly. The reaction became a clear solution and was heated to reflux for two days. Upon completion the volatiles were removed and the product dried in vacuo to give methyl (2S)-2-amino-5-methyl-hexanoate (hydrochloride salt) (13.33 g, 94%) as a white solid. ESI-MS m/z calc. 159.12593, found 160.4 (M+1)⁺; Retention time: 1.11 minutes; LC method T.

### Step 2: Methyl (2S)-2-(benzylamino)-5-methyl-hexanoate

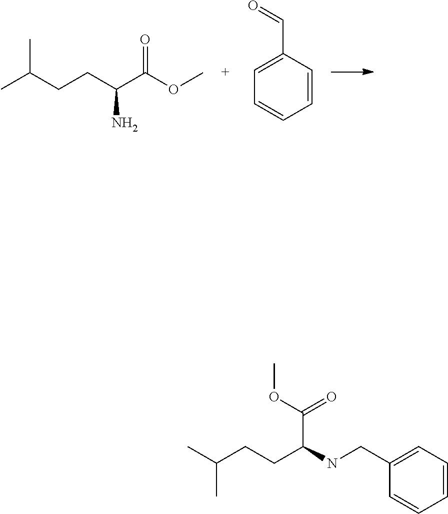

Methyl (2S)-2-amino-5-methyl-hexanoate (hydrochloride salt) (12.3 g, 62.856 mmol) and benzaldehyde (6.3440 g, 6.1 mL, 59.780 mmol) were dissolved in DCE (350 mL) and sodium triacetoxyborohydride (26.68 g, 125.88 mmol) was added portionwise. After 40 minutes the reaction was quenched by the addition of sodium bicarbonate (200 mL). the layers were separated, and the aqueous layer was extracted with DCM three times (100 mL). The combined organic layers were dried over sodium sulfate and concentrated. The crude residue was dry loaded on to silica gel and purified by flash column chromatography using 0-15% Hexanes/EtOAc as an eluent to give methyl (2S)-2-(benzylamino)-5-methyl-hexanoate (11.04 g, 67%) as a clear colorless oil. ESI-MS m/z calc. 249.17288, found 250.2 (M+1)⁺; Retention time: 1.73 minutes; LC method T.

### Step 3: (3S,6R)-4-Benzyl-6-hydroxy-3-isopentyl-1,4-diazepan-2-one

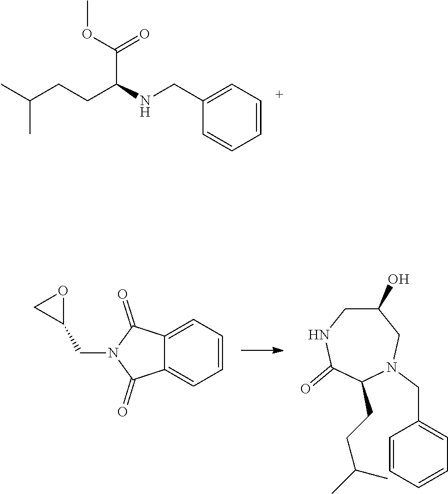 [SMILES: COC(=O)[C@H](CCC(C)C)NCc1ccccc1 ; O=C1c2ccccc2C(=O)N1C[C@@H]1CO1 ; CC(C)CC[C@H]1C(=O)NC[C@@H](O)CN1Cc1ccccc1]

Methyl (2S)-2-(benzylamino)-5-methyl-hexanoate (11.721 g, 47.006 mmol) was dissolved in MeCN (134 mL) and 2-[[(2R)-oxiran-2-yl]methyl]isoindoline-1,3-dione (10.04 g, 49.411 mmol) was added. Next, magnesium perchlorate (15.65 g, 70.115 mmol) was added portionwise and the reaction allowed to stir at room temperature for 3 days. The reaction was diluted with water (200 mL) and extracted three times with DCM (200 mL), the combined organic layers were dried over sodium sulfate and evaporated. The crude residue was dissolved in MeOH (300 mL) and hydrazine hydrate (4.8450 g, 4.75 mL, 96.783 mmol) was added to the solution. The reaction was heated to 65° C. for 24 h, then cooled to room temperature. The solids were filtered off and the filtrate was concentrated. The crude residue was dry loaded on to silica gel and purified by flash column chromatography using 0-30% hexanes in EtOAc as an eluent. The appropriate fractions were collected to give (3S,6R)-4-benzyl-6-hydroxy-3-isopentyl-1,4-diazepan-2-one (6.43 g, 45%) as a colorless oil. ESI-MS m/z calc. 290.19943, found 291.3 (M+1)⁺; Retention time: 1.51 minutes; LC method T.

### Step 4: tert-Butyl (3S,6S)-4-benzyl-6-hydroxy-3-isopentyl-1,4-diazepane-1-carboxylate

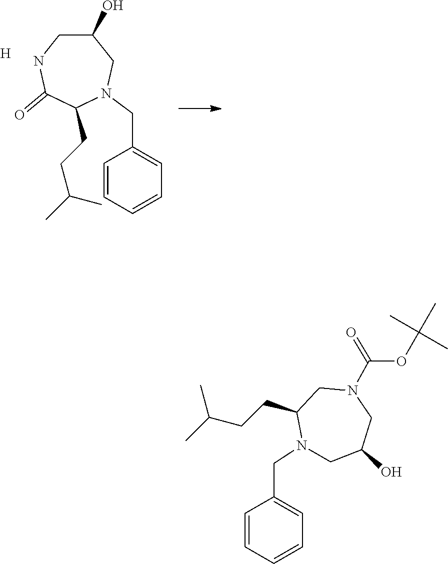

(3S,6R)-4-Benzyl-6-hydroxy-3-isopentyl-1,4-diazepan-2-one (6.43 g, 22.142 mmol) was dissolved in THE (200 mL) and cooled to 0° C. LAH (5.0422 g, 132.85 mmol) was added slowly under a stream of nitrogen. Once complete the reaction was allowed to stir at room temperature for 20 minutes, then warmed to room temp for 1 h, then heated to 65° C. overnight. The reaction was cooled to 0° C. and then 5 mL of DI water, 7 mL 2M NaOH, then 15 mL DI water were added slowly. Sodium sulfate was added, and the reaction stirred for 20 min, then the solids were filtered off and the filtrate concentrated. The crude residue was dissolved in DCM (100 mL) and TEA (2.2405 g, 3.0861 mL, 22.142 mmol) and Boc anhydride (7.2486 g, 33.213 mmol) were added. The reaction was stirred at room temperature for 1 hour, then quenched with brine (100 mL). The aqueous layer was extracted three times with DCM (50 mL). The organic layer was dried over sodium sulfate and concentrated. The crude residue was dry loaded on to silica gel and purified by flash column chromatography with 0-25% Hexane:Acetone. The appropriate fractions were collected to give tert-butyl (3S,6S)-4-benzyl-6-hydroxy-3-isopentyl-1,4-diazepane-1-carboxylate (7.08 g, 81%) as a colorless oil. ESI-MS m/z calc. 376.27258, found 377.5 (M+1)⁺; Retention time: 2.27 minutes; LC method T.

### Step 5: tert-Butyl (3S,6S)-6-hydroxy-3-isopentyl-1,4-diazepane-1-carboxylate

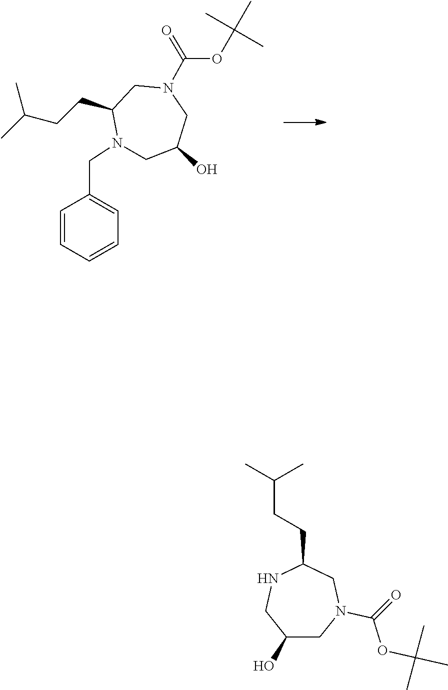 [SMILES: CC(C)CC[C@H]1CN(C(=O)OC(C)(C)C)C[C@@H](O)CN1Cc1ccccc1 ; CC(C)CC[C@H]1CN(C(=O)OC(C)(C)C)C[C@@H](O)CN1]

tert-Butyl (3S,6S)-4-benzyl-6-hydroxy-3-isopentyl-1,4-diazepane-1-carboxylate (7.08 g, 18.803 mmol) and ammonium formate (4.76 g, 75.489 mmol) were dissolved in MeOH (200 mL) and palladium on carbon (3.09 g, 10% w/w, 2.9036 mmol) was added. The mixture was heated to 65° C. for 1 hour then Celite was added and the solids were filtered. The filtrate was concentrated and dissolved in ethyl acetate (75 mL) and aqueous ammonium chloride (75 mL). The aqueous layer was extracted to give tert-butyl (3S,6S)-6-hydroxy-3-isopentyl-1,4-diazepane-1-carboxylate (2.9 g, 51%) as a white solid. ESI-MS m/z calc. 286.2256, found 287.1 (M+1)⁺; Retention time: 1.71 minutes (LC method W). ¹H NMR (500 MHz, DMSO-d₆) δ 3.85-3.66 (m, 3H), 2.96-2.82 (m, 1H), 2.81-2.66 (m, 1H), 2.62-2.52 (m, 1H), 2.42 (d, J=9.1 Hz, 1H), 1.54-1.43 (m, 1H), 1.37 (s, 9H), 1.32-1.08 (m, 4H), 0.85 (dd, J=6.6, 1.9 Hz, 6H)

The aqueous layer was basified with sodium bicarbonate and extracted three times with EtOAc (40 mL) to give a second lot of tert-butyl (3S,6S)-6-hydroxy-3-isopentyl-1,4-diazepane-1-carboxylate (2.16 g, 38%) as a white solid. ESI-MS m/z calc. 286.2256, found 287.1 (M+1)⁺; Retention time: 1.71 minutes (LC method W). ¹H NMR (500 MHz, DMSO-d₆) δ 3.85-3.66 (m, 3H), 2.96-2.82 (m, 1H), 2.81-2.66 (m, 1H), 2.62-2.52 (m, 1H), 2.42 (d, J=9.1 Hz, 1H), 1.54-1.43 (m, 1H), 1.37 (s, 9H), 1.32-1.08 (m, 4H), 0.85 (dd, J=6.6, 1.9 Hz, 6H).

### Step 6: tert-Butyl (3S,6S)-4-[3-[[4-chloro-6-(2,6-dimethylphenyl)pyrimidin-2-yl]sulfamoyl]benzoyl]-6-hydroxy-3-isopentyl-1,4-diazepane-1-carboxylate

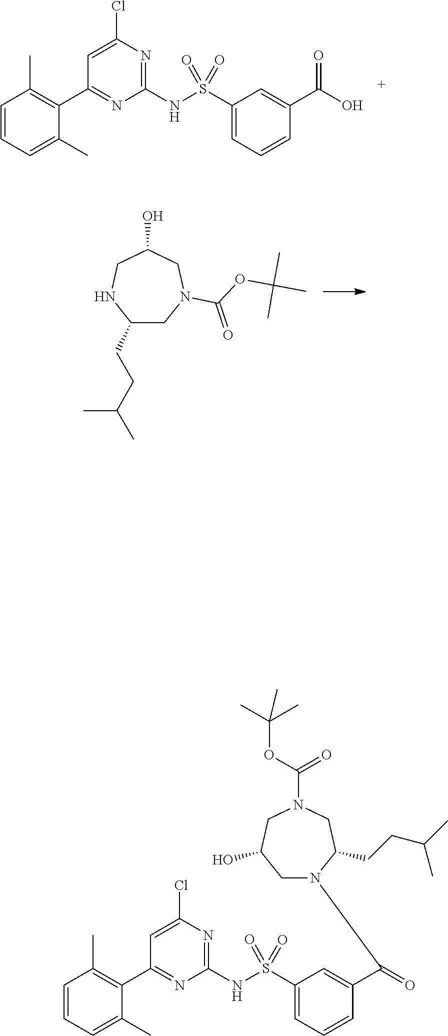 [SMILES: Cc1cccc(C)c1-c1cc(Cl)nc(NS(=O)(=O)c2cccc(C(=O)O)c2)n1 ; CC(C)CC[C@H]1CN(C(=O)OC(C)(C)C)C[C@@H](O)CN1 ; Cc1cccc(C)c1-c1cc(Cl)nc(NS(=O)(=O)c2cccc(C(=O)N3C[C@H](O)CN(C(=O)OC(C)(C)C)C[C@@H]3CCC(C)C)c2)n1]

A 100 mL flask was charged under nitrogen with tert-butyl (3S,6S)-6-hydroxy-3-isopentyl-1,4-diazepane-1-carboxylate (1.03 g, 3.596 mmol), anhydrous DMF (20 mL) and 3-[[4-chloro-6-(2,6-dimethylphenyl)pyrimidin-2-yl]sulfamoyl]benzoic acid (1.256 g, 3.006 mmol). After dissolution of the reagents, the mixture was cooled down in an ice bath. DIEA (3.5 mL, 20.09 mmol) and HATU (1.44 g, 3.787 mmol) were added and the mixture was stirred at 0° C. for 5 hours. The reaction was quenched by being poured in citric acid (110 mL of 10% w/v, 57.25 mmol) (10% aqueous) under vigorous stirring and cooled in ice. The resulting white solid was filtered. The solid was dissolved in DCM. After concentration, it was purified by flash chromatography on silica gel (120 g column) using a gradient of methanol (0 to 5% over 30 min) in dichloromethane. The product eluted around 3-4% methanol. Evaporation of the solvents gave tert-butyl (3S,6S)-4-[3-[[4-chloro-6-(2,6-dimethylphenyl)pyrimidin-2-yl]sulfamoyl]benzoyl]-6-hydroxy-3-isopentyl-1,4-diazepane-1-carboxylate (1.433 g, 69%) as a white foamy solid. ESI-MS m/z calc. 685.2701, found 686.34 (M+1)⁺; Retention time: 2.06 minutes; LC method A.

### Step 7: tert-Butyl (16S,20S)-12-(2,6-dimethylphenyl)-20-(3-methylbutyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (Compound 129)

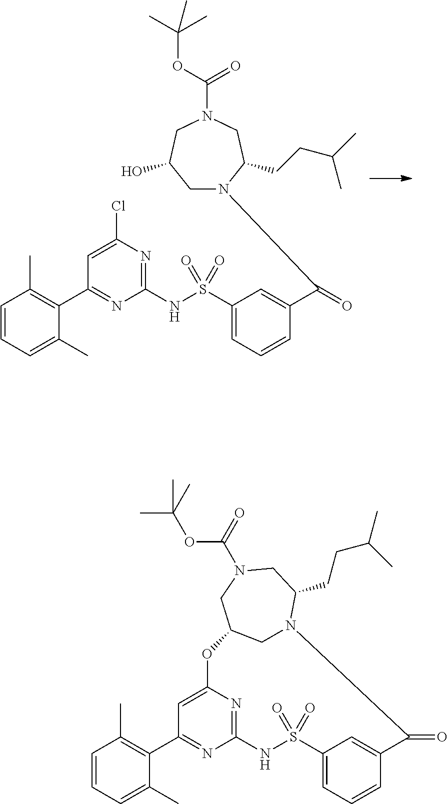 [SMILES: Cc1cccc(C)c1-c1cc(Cl)nc(NS(=O)(=O)c2cccc(C(=O)N3C[C@H](O)CN(C(=O)OC(C)(C)C)C[C@@H]3CCC(C)C)c2)n1 ; Cc1cccc(C)c1-c1cc2nc(n1)NS(=O)(=O)c1cccc(c1)C(=O)N1C[C@@H](CN(C(=O)OC(C)(C)C)C[C@@H]1CCC(C)C)O2]

A 100 mL flask was charged under nitrogen with tert-butyl (3S,6S)-4-[3-[[4-chloro-6-(2,6-dimethylphenyl)pyrimidin-2-yl]sulfamoyl]benzoyl]-6-hydroxy-3-isopentyl-1,4-diazepane-1-carboxylate (1.433 g, 2.088 mmol) and anhydrous DMF (70 mL). The mixture was cooled down in ice. NaH (677 mg of 60% w/w, 16.93 mmol) (60% mineral oil dispersion) was added in one portion. The mixture was stirred under nitrogen at 0° C. for 10 minutes. The ice bath was removed, and the reaction was vigorously stirred under nitrogen for 3.5 hours. The reaction mixture was slowly poured into an ice-cold citric acid (300 mL of 10% w/v, 156.1 mmol) 10% aqueous solution under stirring. The resulting solid suspension was extracted with EtOAc (3×75 mL). After drying over sodium sulfate, evaporation of the solvents gave a residue that was dissolved in DCM and purified by flash chromatography on silica gel (80 g column) using a gradient of MeOH (0 to 5% over 30 min) in dichloromethane. The product around 2-3% MeOH. Evaporation of the solvents and several cycle of trituration/evaporation in DCM/hexanes gave tert-butyl (16S,20S)-12-(2,6-dimethylphenyl)-20-(3-methylbutyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (739 mg, 53%) as a white solid. ESI-MS m/z calc. 649.2934, found 650.37 (M+1)⁺; Retention time: 2.05 minutes; LC method A.

### Step 8: (16R,20S)-12-(2,6-Dimethylphenyl)-20-(3-methylbutyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 130)

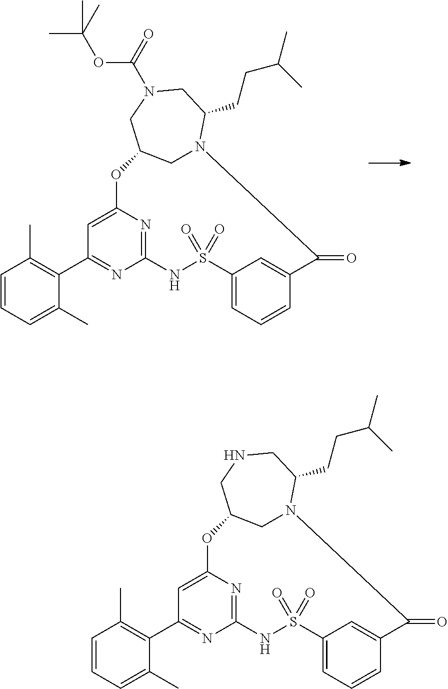 [SMILES: Cc1cccc(C)c1-c1cc2nc(n1)NS(=O)(=O)c1cccc(c1)C(=O)N1C[C@@H](CN(C(=O)OC(C)(C)C)C[C@@H]1CCC(C)C)O2 ; Cc1cccc(C)c1-c1cc2nc(n1)NS(=O)(=O)c1cccc(c1)C(=O)N1C[C@@H](CNC[C@@H]1CCC(C)C)O2]

A 100 mL flask containing tert-butyl (16S,20S)-12-(2,6-dimethylphenyl)-20-(3-methylbutyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (723 mg, 1.090 mmol) was charged with DCM (7 mL). HCl (5 mL of 4 M, 20.00 mmol) (4 M dioxane solution) was added and the mixture was stirred at room temperature for 2 hours. The volatiles were removed by evaporation and the residue was triturated in DCM/hexanes and the solvents were evaporated. The operation was repeated until a solid was obtained. Drying under vacuum gave (16R,20S)-12-(2,6-dimethylphenyl)-20-(3-methylbutyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (655 mg, 101%) as a white solid. ¹H NMR (500 MHz, DMSO-d₆+10% D₂O) δ 8.63 (s, 1H), 7.87 (d, J=7.9 Hz, 1H), 7.66 (t, J=7.7 Hz, 1H), 7.55 (d, J=7.5 Hz, 1H), 7.27 (t, J=7.7 Hz, 1H), 7.12 (d, J=7.7 Hz, 2H), 6.20 (s, 1H), 5.95-5.82 (m, 1H), 4.73-4.59 (m, 1H), 3.82-3.75 (m, 1H overlapped with water), 3.61-3.41 (m, 2H), 3.31-3.19 (m, 2H), 3.13 (t, J=11.7 Hz, 1H), 2.02 (broad s, 6H), 1.71-1.48 (m, 3H), 1.27-1.10 (m, 2H), 0.88 (d, J=6.5 Hz, 6H). ESI-MS m/z calc. 549.24097, found 550.43 (M+1)⁺; Retention time: 1.23 minutes; LC method A.

### Step 9: (16R,20S)-12-(2,6-Dimethylphenyl)-18-methyl-20-(3-methylbutyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 131)

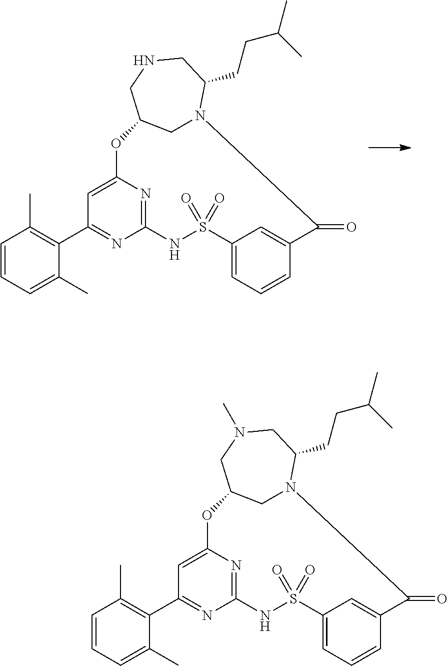 [SMILES: Cc1cccc(C)c1-c1cc2nc(n1)NS(=O)(=O)c1cccc(c1)C(=O)N1C[C@@H](CNC[C@@H]1CCC(C)C)O2 ; Cc1cccc(C)c1-c1cc2nc(n1)NS(=O)(=O)c1cccc(c1)C(=O)N1C[C@@H](CN(C)C[C@@H]1CCC(C)C)O2]

(16R,20S)-12-(2,6-Dimethylphenyl)-20-(3-methylbutyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (22 mg, 0.03678 mmol) was dissolved in formic acid (250 μL) (88% aqueous) and combined with aqueous formaldehyde (900 μL, 32.67 mmol) (37% aqueous) and heated to 90° C. for 4 hours in a screwcap vial. The reaction mixture was then partially concentrated by blowing nitrogen, diluted with methanol. The solution was microfiltered through a Whatman 0.45 μM PTFE syringe filter disc and purified by reverse phase preparative HPLC (Cis) using a gradient of acetonitrile in water (1 to 99% over 15 min) and HCl as a modifier to give (16R,20S)-12-(2,6-dimethylphenyl)-18-methyl-20-(3-methylbutyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (16.8 mg, 75%) as a white solid. ESI-MS m/z calc. 563.25665, found 564.69 (M+1)⁺; Retention time: 1.2 minutes; LC method A.

### Example 66: Preparation of Compound 132 and Compound 133

### Step 1: Methyl (2S)-2-(benzylamino)-3-methyl-butanoate

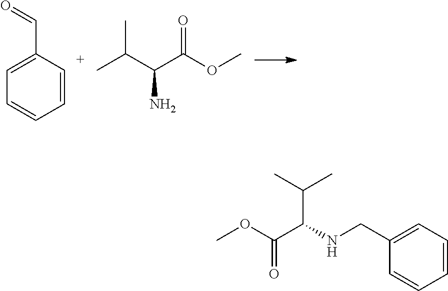

To a solution of methyl (2S)-2-amino-3-methyl-butanoate (hydrochloride salt) (45 g, 263.07 mmol), TEA (26.862 g, 37 mL, 265.46 mmol), benzaldehyde (28.208 g, 27 mL, 264.48 mmol) in DCE (500 mL) was added sodium triacetoxyborohydride (112 g, 528.45 mmol). The mixture was stirred at room temperature for overnight. Afterwards, DCM (300 mL) was added and the organic layer was washed with brine (2×800 mL). The organic layer was extracted with 1M HCl (800 mL). The aqueous layer was separated, basified with 2M sodium hydroxide solution, then extracted with ethyl acetate (2×800 mL). The organic layer was washed with brine (800 mL), dried over sodium sulfate, filtered and concentrated in vacuo to afford methyl (2S)-2-(benzylamino)-3-methyl-butanoate (38.12 g, 64%) as a colorless oil. ESI-MS m/z calc. 221.14159, found 222.4 (M+1)⁺; Retention time: 1.69 minutes; LC method T.

### Step 2: (3S,6R)-4-Benzyl-6-hydroxy-3-isopropyl-1,4-diazepan-2-one

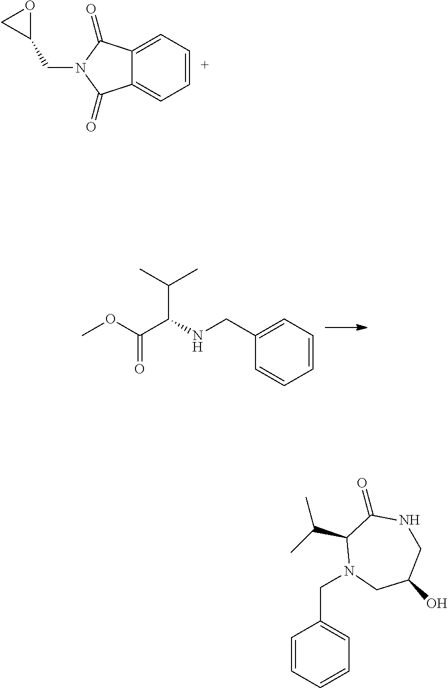 [SMILES: O=C1c2ccccc2C(=O)N1C[C@@H]1CO1 ; COC(=O)[C@@H](NCc1ccccc1)C(C)C ; CC(C)[C@H]1C(=O)NC[C@@H](O)CN1Cc1ccccc1]

To a solution of 2-[[(2R)-oxiran-2-yl]methyl]isoindoline-1,3-dione (22 g, 107.19 mmol) in ACN (140 mL) was added methyl (2S)-2-(benzylamino)-3-methyl-butanoate (24.205 g, 107.19 mmol) and magnesium perchlorate (40 g, 164.87 mmol). The reaction mixture was stirred at room temperature before being diluted with water (250 mL) and removed ACN in vacuo. Then the cloudy mixture was extracted with DCM (3×250 mL). The combined organic layers were washed with brine (500 mL), dried over anhydrous sodium sulfate and concentrated under vacuum. The organic residue (48 g) was dissolved in methanol (500 mL). Hydrazine hydrate (15.023 g, 20 mL, 219.07 mmol) was added to the reaction mixture and then it was stirred at 65° C. for 40 hours. The reaction was cooled to room temperature and the white solid was filtered off. The filtrate was concentrated and then diluted with 1N NaOH (800 mL) before being extracted with ethyl acetate (2×600 mL). The combined organic layers were washed with brine (600 mL), dried over sodium sulfate and concentrated before being purified by silica gel chromatography eluting 0-10% DCM-MeOH to give (3S,6R)-4-benzyl-6-hydroxy-3-isopropyl-1,4-diazepan-2-one (11.44 g, 40%) as a white foam. ESI-MS m/z calc. 262.16812, found 263.2 (M+1)⁺; Retention time: 1.89 minutes; LC method T.

### Step 3: (2S,6R)-1-Benzyl-2-isopropyl-1,4-diazepan-6-ol

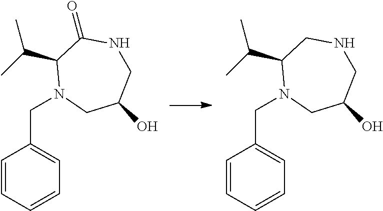 [SMILES: CC(C)[C@H]1C(=O)NC[C@@H](O)CN1Cc1ccccc1 ; CC(C)[C@H]1CNC[C@@H](O)CN1Cc1ccccc1]

Into a solution of (3S,6R)-4-benzyl-6-hydroxy-3-isopropyl-1,4-diazepan-2-one (11.44 g, 42.734 mmol) in THE (300 mL) was added LAH (20 g, 500.60 mmol). The reaction was stirred at 60° C. for 20 hours before being cooled to room temperature. The reaction was quenched with water (20 mL), 15% NaOH (aqueous) (20 mL) and water (60 mL) at 0° C. subsequently. The reaction mixture was stirred for another 30 minutes, then it was filtered through a pad of Celite. The filter cake was washed with THE (3×150 mL). The combined filtrate was concentrated under vacuum to give (2S,6R)-1-benzyl-2-isopropyl-1,4-diazepan-6-ol (11.6 g, 104%). ESI-MS m/z calc. 248.18886, found 249.4 (M+1)⁺; Retention time: 1.54 minutes; LC method T.

### Step 4: tert-Butyl (3S,6S)-4-benzyl-6-hydroxy-3-isopropyl-1,4-diazepane-1-carboxylate

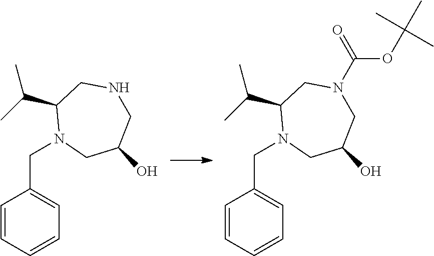 [SMILES: CC(C)[C@H]1CNC[C@@H](O)CN1Cc1ccccc1 ; CC(C)[C@H]1CN(C(=O)OC(C)(C)C)C[C@@H](O)CN1Cc1ccccc1]

To a solution of (2S,6R)-1-benzyl-2-isopropyl-1,4-diazepan-6-ol (11.6 g, 42.736 mmol) in DCM (140 mL) was added Boc anhydride (15 g, 66.668 mmol) and triethylamine (13.068 g, 18 mL, 129.14 mmol) at room temperature and stirred overnight. The reaction was quenched with Saturated sodium bicarbonate (200 mL) and brine (100 mL). The two layers were separated, and the aqueous layer was extracted with DCM (2×200 mL). The combined organic layers were dried over anhydrous sodium sulfate and concentrated under vacuum. The residue was purified by silica gel chromatography using 0-60% hexanes-ethyl acetate to give tert-butyl (3S,6S)-4-benzyl-6-hydroxy-3-isopropyl-1,4-diazepane-1-carboxylate (12.9 g, 85%) as a clear oil. ESI-MS m/z calc. 348.2413, found 349.5 (M+1)⁺; Retention time: 2.4 minutes; LC method T.

### Step 5: tert-Butyl (3S,6S)-6-hydroxy-3-isopropyl-1,4-diazepane-1-carboxylate

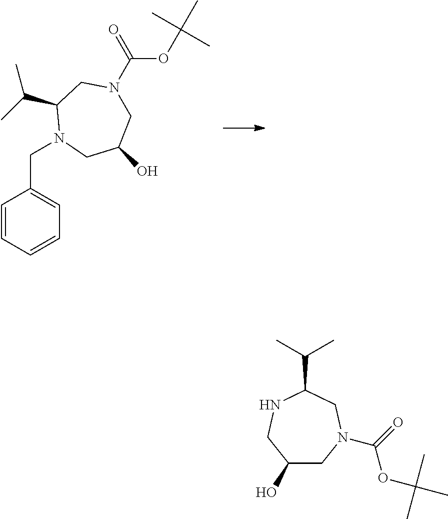 [SMILES: CC(C)[C@H]1CN(C(=O)OC(C)(C)C)C[C@@H](O)CN1Cc1ccccc1 ; CC(C)[C@H]1CN(C(=O)OC(C)(C)C)C[C@@H](O)CN1]

To a solution of tert-butyl (3S,6S)-4-benzyl-6-hydroxy-3-isopropyl-1,4-diazepane-1-carboxylate (12.9 g, 36.278 mmol) in HOAc (3.1680 g, 3 mL, 52.754 mmol), ethyl acetate (80 mL) and methanol (20 mL) was added 10% palladium on carbon (1.5 g). The mixture was in a Parr shaker at 60 psi for 1.5 hours. The reaction mixture was filtered through Celite pad. The filtrate was concentrated under vacuum. The residue was added Saturated sodium bicarbonate (200 mL) and DCM (200 mL). The two layers were separated, and the aqueous layer was extracted with DCM (2×200 mL). The combined organic layers were washed with brine (500 mL), dried over anhydrous sodium sulfate and concentrated under vacuum to give tert-butyl (3S,6S)-6-hydroxy-3-isopropyl-1,4-diazepane-1-carboxylate (8.8 g, 92%) as a light color gel. ¹H NMR (500 MHz, DMSO-d₆) δ 3.96-3.68 (m, 3H), 2.98-2.86 (m, 1H), 2.81-2.65 (m, 2H), 2.62-2.53 (m, 1H), 2.39-2.27 (m, 1H), 1.59 (tt, J=12.7, 6.5 Hz, 1H), 1.38 (s, 9H), 0.88 (dt, J=13.0, 6.8 Hz, 6H). ESI-MS m/z calc. 258.19434, found 259.2 (M+1)⁺; Retention time: 1.37 minutes; LC method W.

### Step 6: tert-Butyl (3S,6S)-4-[3-[[4-chloro-6-(2,6-dimethylphenyl)pyrimidin-2-yl]sulfamoyl]benzoyl]-6-hydroxy-3-isopropyl-1,4-diazepane-1-carboxylate

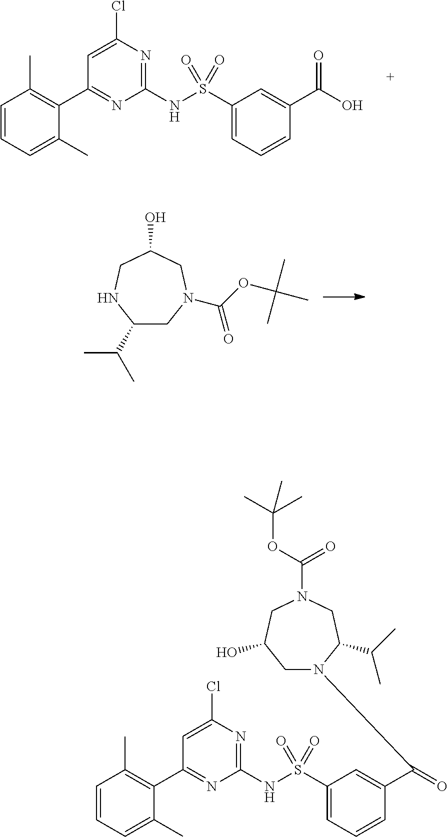 [SMILES: Cc1cccc(C)c1-c1cc(Cl)nc(NS(=O)(=O)c2cccc(C(=O)O)c2)n1 ; CC(C)[C@H]1CN(C(=O)OC(C)(C)C)C[C@@H](O)CN1 ; Cc1cccc(C)c1-c1cc(Cl)nc(NS(=O)(=O)c2cccc(C(=O)N3C[C@H](O)CN(C(=O)OC(C)(C)C)C[C@@H]3C(C)C)c2)n1]

A 100 mL flask was charged under nitrogen with tert-butyl (3S,6S)-6-hydroxy-3-(1-methylethyl)-1,4-diazepane-1-carboxylate (1.765 g, 6.832 mmol), anhydrous DMF (35 mL) and 3-[[4-chloro-6-(2,6-dimethylphenyl)pyrimidin-2-yl]sulfamoyl]benzoic acid (2.418 g, 5.787 mmol). After dissolution of the reagents, the mixture was cooled down in an ice bath. DIEA (6.8 mL, 39.04 mmol) and HATU (2.64 g, 6.943 mmol) were added and the mixture was stirred at 0° C. for 30 min, then at room temperature for 6.5 hours. The reaction was quenched by being poured in citric acid (200 mL of 10% w/v, 104.1 mmol) (10% aqueous) under vigorous stirring and cooled in ice. The resulting off-white solid was filtered. The wet solid was dissolved in DCM and the solution was dried over sodium sulfate. After concentration, it was purified by flash chromatography on silica gel (220 g column) using a gradient of methanol (0 to 5% over 30 min) in dichloromethane. The product eluted around 3-4% methanol. Evaporation of the solvents gave 1.075 g of an off-white foamy solid that was about 70% pure. The material was purified a second time using a 120 g column and the same solvent gradient. Evaporation of the solvents gave tert-butyl (3S,6S)-4-[3-[[4-chloro-6-(2,6-dimethylphenyl)pyrimidin-2-yl]sulfamoyl]benzoyl]-6-hydroxy-3-isopropyl-1,4-diazepane-1-carboxylate (773 mg, 20%) as a white foamy solid. ESI-MS m/z calc. 657.2388, found 658.29 (M+1)⁺; Retention time: 1.92 minutes; LC method A.

### Step 7: tert-Butyl (16S,20S)-12-(2,6-dimethylphenyl)-2,8,8-trioxo-20-(propan-2-yl)-15-oxa-8λ⁶-thia-1,9,11,18,22-pentaazatetracyclo[14.4.1.13,7.110,14]tricosa-3(23),4,6,10(22),11,13-hexaene-18-carboxylate (Compound 132)

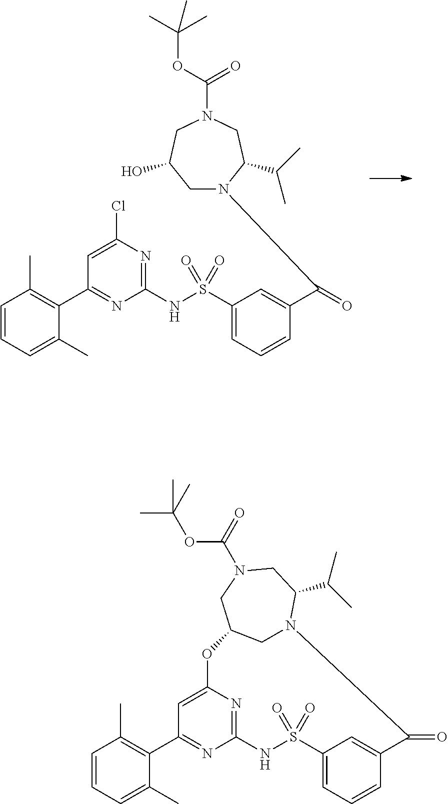 [SMILES: Cc1cccc(C)c1-c1cc(Cl)nc(NS(=O)(=O)c2cccc(C(=O)N3C[C@H](O)CN(C(=O)OC(C)(C)C)C[C@@H]3C(C)C)c2)n1 ; Cc1cccc(C)c1-c1cc2nc(n1)NS(=O)(=O)c1cccc(c1)C(=O)N1C[C@@H](CN(C(=O)OC(C)(C)C)C[C@@H]1C(C)C)O2]

A 250 mL flask was charged under nitrogen with tert-butyl (3S,6S)-4-[3-[[4-chloro-6-(2,6-dimethylphenyl)pyrimidin-2-yl]sulfamoyl]benzoyl]-6-hydroxy-3-isopropyl-1,4-diazepane-1-carboxylate (773 mg, 1.174 mmol) and anhydrous DMF (40 mL). The mixture was cooled down in ice. NaH (375 mg of 60% w/w, 9.376 mmol) (60% mineral oil dispersion) was added in two equal portions, added 3 minutes after each other. The mixture was stirred under nitrogen at 0° C. for 10 minutes. The ice bath was removed, and the reaction was vigorously stirred under nitrogen for 3 hours. The reaction mixture was slowly poured into an ice-cold citric acid (150 mL of 10% w/v, 78.07 mmol) aqueous solution under stirring. The resulting solid suspension was extracted with EtOAc (3×75 mL). After drying over sodium sulfate, evaporation of the solvents gave a residue that was dissolved in DCM and purified by flash chromatography on silica gel (80 g column) using a gradient of MeOH (0 to 5% over 30 min) in dichloromethane. The product eluted around 3-4% MeOH. Evaporation of the solvents and several cycles of trituration/evaporation in DCM/hexanes gave tert-butyl (16S,20S)-12-(2,6-dimethylphenyl)-2,8,8-trioxo-20-(propan-2-yl)-15-oxa-8λ⁶-thia-1,9,11,18,22-pentaazatetracyclo[14.4.1.13,7.110,14]tricosa-3(23),4,6,10(22),11,13-hexaene-18-carboxylate (344 mg, 46%) as a white solid. ESI-MS m/z calc. 621.2621, found 622.29 (M+1)⁺; Retention time: 1.8 minutes; LC method A.

### Step 8: (16R,20S)-12-(2,6-Dimethylphenyl)-20-(propan-2-yl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 133)

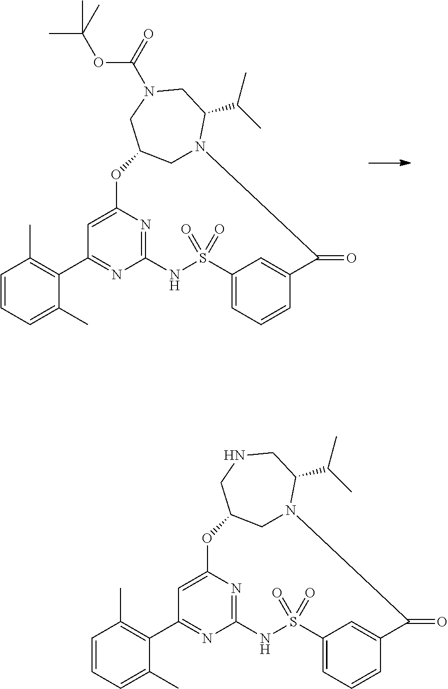 [SMILES: Cc1cccc(C)c1-c1cc2nc(n1)NS(=O)(=O)c1cccc(c1)C(=O)N1C[C@@H](CN(C(=O)OC(C)(C)C)C[C@@H]1C(C)C)O2 ; Cc1cccc(C)c1-c1cc2nc(n1)NS(=O)(=O)c1cccc(c1)C(=O)N1C[C@@H](CNC[C@@H]1C(C)C)O2]

A 100 mL flask containing tert-butyl (16S,20S)-12-(2,6-dimethylphenyl)-2,8,8-trioxo-20-(propan-2-yl)-15-oxa-8λ⁶-thia-1,9,11,18,22-pentaazatetracyclo[14.4.1.13,7.110,14]tricosa-3(23),4,6,10(22),11,13-hexaene-18-carboxylate (337 mg, 0.5312 mmol) was charged under nitrogen with DCM (3 mL). HCl (3 mL of 4 M, 12.00 mmol) (4 M dioxane solution) was added and the mixture was stirred at room temperature for 2 hours. The volatiles were removed by evaporation and the residue was triturated in DCM/hexanes and the solvents were evaporated. The operation was repeated until a solid was obtained. Drying under vacuum gave (16R,20S)-12-(2,6-dimethylphenyl)-20-(propan-2-yl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (341 mg, 109%) as a white solid. ¹H NMR (500 MHz, DMSO-d₆₊₁₀% D₂O) δ 8.64 (s, 1H), 7.91-7.78 (m, 1H), 7.65 (t, J=8.4 Hz, 1H), 7.51 (d, J=7.5 Hz, 1H), 7.32-7.21 (m, 1H), 7.12 (d, J=7.6 Hz, 2H), 6.18 (s, 1H), 5.85 (s, 1H), 4.63 (s, 1H), 3.90-3060 (m, 3H overlapped with water), 3.49 (t, J=13.3 Hz, 1H), 3.36-3.16 (m, 3H), 2.02 (broad s, 6H), 0.99-0.83 (m, 6H). ESI-MS m/z calc. 521.20966, found 522.3 (M+1)⁺; Retention time: 1.0 minutes; LC method A.

### Example 67: Preparation of Compound 134, Compound 135, and Compound 136

### Step 1: Methyl (2S)-2-(benzylamino)-4,4-dimethyl-pentanoate

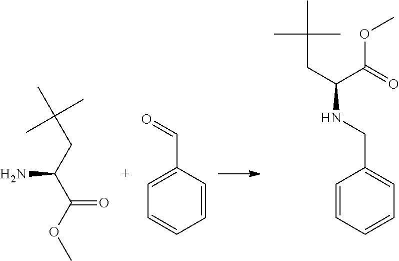

To a stirring solution of methyl (2S)-2-amino-4,4-dimethyl-pentanoate (hydrochloride salt) (25.65 g, 124.52 mmol) in anhydrous 1,2-dichloroethane (600 mL) at room temperature under nitrogen was added benzaldehyde (13.214 g, 12.706 mL, 124.52 mmol). The reaction mixture was stirred for 15 minutes and sodium triacetoxyborohydride (65.977 g, 311.30 mmol) was added portionwise. After the addition was complete, the reaction mixture was stirred at room temperature for 45 minutes. The reaction was quenched with saturated aqueous sodium bicarbonate (500 mL) and two layers were separated. The aqueous layer was extracted with DCM (2×150 mL). The combined organic layers were washed with brine (200 mL), dried over anhydrous sodium sulfate and concentrated. The crude was purified by silica gel chromatography using 0-5% hexanes-ethyl acetate to afford methyl (2S)-2-(benzylamino)-4,4-dimethyl-pentanoate (22.82 g, 70%) as a colorless oil. ESI-MS m/z calc. 249.17288, found 250.2 (M+1)⁺; Retention time: 2.31 minutes; LC method S.

### Step 2: (3S,6R)-4-Benzyl-3-(2,2-dimethylpropyl)-6-hydroxy-1,4-diazepan-2-one

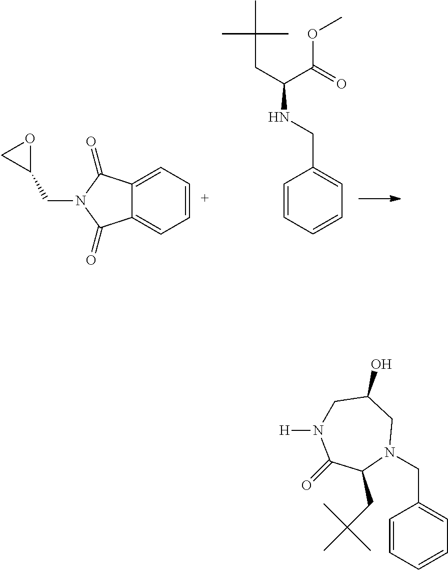 [SMILES: O=C1c2ccccc2C(=O)N1C[C@@H]1CO1 ; COC(=O)[C@H](CC(C)(C)C)NCc1ccccc1 ; CC(C)(C)C[C@H]1C(=O)NC[C@@H](O)CN1Cc1ccccc1]

To a stirring solution of methyl (2S)-2-(benzylamino)-4,4-dimethyl-pentanoate (22.82 g, 91.518 mmol) and 2-[[(2R)-oxiran-2-yl]methyl]isoindoline-1,3-dione (19.526 g, 96.094 mmol) in acetonitrile (170 mL) at room temperature, was added magnesium perchlorate (30.642 g, 137.28 mmol). The reaction mixture was heated to 30° C. for 24 hours. The reaction was quenched with water (500 mL) and the product was extracted with DCM (3×250 mL). The combined organic layers were washed with brine (100 mL), dried over anhydrous sodium sulfate and concentrated. The obtained residue was dissolved in methanol (500 mL) at room temperature and hydrazine hydrate (9.1631 g, 183.04 mmol) was added. The reaction mixture was heated to 65° C. for 24 hours. After cooling to room temperature, the white precipitate was filtered off and the filtrate was concentrated under vacuum. The obtained residue was treated with 1 M aqueous NaOH (500 mL) and the product was extracted with ethyl acetate (3×250 mL). The combined organic layers were washed with brine (100 mL), dried over anhydrous sodium sulfate and concentrated. The crude was purified by silica gel chromatography using 0-5% DCM-methanol to afford (3S,6R)-4-benzyl-3-(2,2-dimethylpropyl)-6-hydroxy-1,4-diazepan-2-one (21.75 g, 79%) as a white foam. ¹H NMR (250 MHz, CDCl₃) δ 7.41-7.17 (m, 5H), 6.28 (s, 1H), 3.97-3.69 (m, 3H), 3.68-3.44 (m, 2H), 3.43-3.24 (m, 1H), 3.21-3.05 (m, 1H), 2.98-2.79 (m, 1H), 2.24 (s, 1H), 2.07-1.84 (m, 1H), 1.75-1.52 (m, 1H), 0.96 (s, 9H). ESI-MS m/z calc. 290.19943, found 291.0 (M+1)⁺; Retention time: 1.84 minutes; LC method T.

### Step 3: (2S,6R)-1-Benzyl-2-(2,2-dimethylpropyl)-1,4-diazepan-6-ol

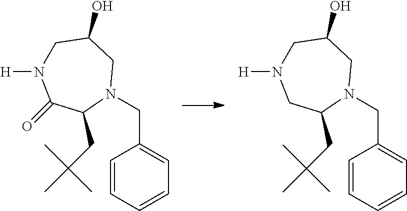 [SMILES: CC(C)(C)C[C@H]1C(=O)NC[C@@H](O)CN1Cc1ccccc1 ; CC(C)(C)C[C@H]1CNC[C@@H](O)CN1Cc1ccccc1]

To a stirring solution of (3S,6R)-4-benzyl-3-(2,2-dimethylpropyl)-6-hydroxy-1,4-diazepan-2-one (10.1 g, 34.779 mmol) in anhydrous THE (300 mL) at 0° C. under nitrogen was added portionwise LAH (7.9199 g, 208.67 mmol). After the addition was complete, the reaction mixture was stirred at 0° C. for 15 minutes, then heated to 45° C. for 24 hours. The reaction mixture was cooled to 0° C. and quenched following a Fieser workup procedure. Salts were filtered off and washed with THE (2×100 mL). The combined filtrate was concentrated under vacuum to afford (2S,6R)-1-benzyl-2-(2,2-dimethylpropyl)-1,4-diazepan-6-ol (9.86 g, 94%) as white solid. The product was carried to the next step without further purification. ESI-MS m/z calc. 276.22015, found 277.6 (M+1)⁺; Retention time: 1.54 minutes; LC method S.

### Step 4: tert-Butyl (3S,6S)-4-benzyl-3-(2,2-dimethylpropyl)-6-hydroxy-1,4-diazepane-1-carboxylate

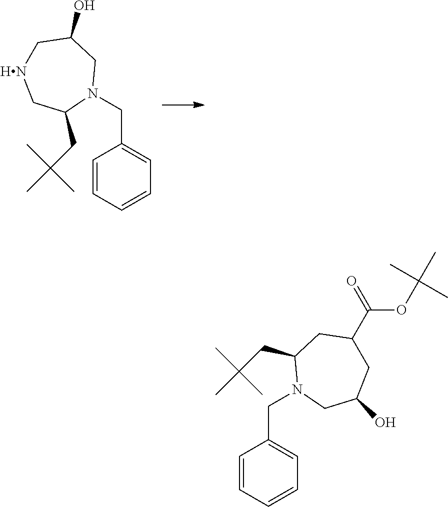

To a stirring solution of (2S,6R)-1-benzyl-2-(2,2-dimethylpropyl)-1,4-diazepan-6-ol (9.86 g, 33.887 mmol) in DCM (120 mL) at 0° C. was added TEA (5.1436 g, 7.0848 mL, 50.831 mmol), followed by Boc anhydride (11.094 g, 50.831 mmol). The reaction mixture was stirred at this temperature for 1 hour. The reaction was quenched cold with brine (200 mL). After warming up to room temperature, two layers were separated. The aqueous layer was extracted with DCM (2×100 mL). The combined organic layers were dried over anhydrous sodium sulfate and concentrated. The crude was purified by silica gel chromatography using 0-25% hexanes-ethyl acetate to afford tert-butyl (3S,6S)-4-benzyl-3-(2,2-dimethylpropyl)-6-hydroxy-1,4-diazepane-1-carboxylate (12.52 g, 93%) as viscous colorless oil. ESI-MS m/z calc. 376.27258, found 377.3 (M+1)⁺; Retention time: 3.44 minutes; LC method S.

### Step 5: tert-Butyl (3S,6S)-3-(2,2-dimethylpropyl)-6-hydroxy-1,4-diazepane-1-carboxylate

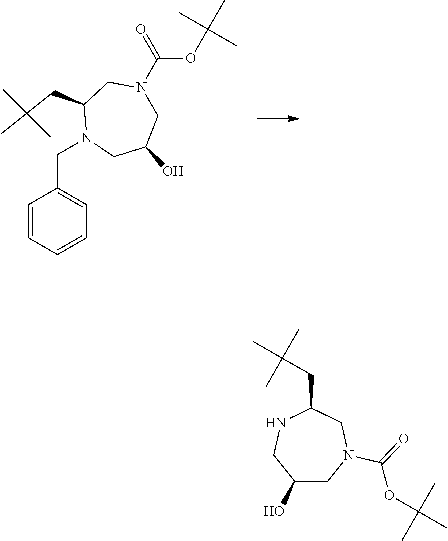 [SMILES: CC(C)(C)C[C@H]1CN(C(=O)OC(C)(C)C)C[C@@H](O)CN1Cc1ccccc1 ; CC(C)(C)C[C@H]1CN(C(=O)OC(C)(C)C)C[C@@H](O)CN1]

To a stirring solution of tert-butyl (3S,6S)-4-benzyl-3-(2,2-dimethylpropyl)-6-hydroxy-1,4-diazepane-1-carboxylate (12.52 g, 33.251 mmol) in anhydrous methanol (325 mL) at room temperature under nitrogen was added palladium on carbon (5.3078 g, 10% w/w, 4.9876 mmol), followed by ammonium formate (8.3864 g, 133.00 mmol). The reaction mixture was heated to 65 C for 2 hours. After cooling to room temperature, the reaction mixture was filtered through a pad of Celite and the solid was washed with methanol (2×80 mL). The combined filtrate was concentrated under vacuum to afford tert-butyl (3S,6S)-3-(2,2-dimethylpropyl)-6-hydroxy-1,4-diazepane-1-carboxylate (9.74 g, 97%) as white solid. ¹H NMR (500 MHz, DMSO-d6) δ 3.90-3.66 (m, 3H), 2.98-2.84 (m, 1H), 2.81-2.53 (m, 4H), 2.48-2.39 (m, 2H), 1.38 (s, 9H), 1.26-1.14 (m, 1H), 1.13-1.04 (m, 1H), 0.89 (s, 9H). ESI-MS m/z calc. 286.22565, found 287.1 (M+1)⁺; Retention time: 1.59 minutes; LC method T.

### Step 6: 3-[[4-[[(3S,6S)-1-tert-Butoxycarbonyl-3-(2,2-dimethylpropyl)-1,4-diazepan-6-yl]oxy]-6-(2,6-dimethylphenyl)pyrimidin-2-yl]sulfamoyl]benzoic acid

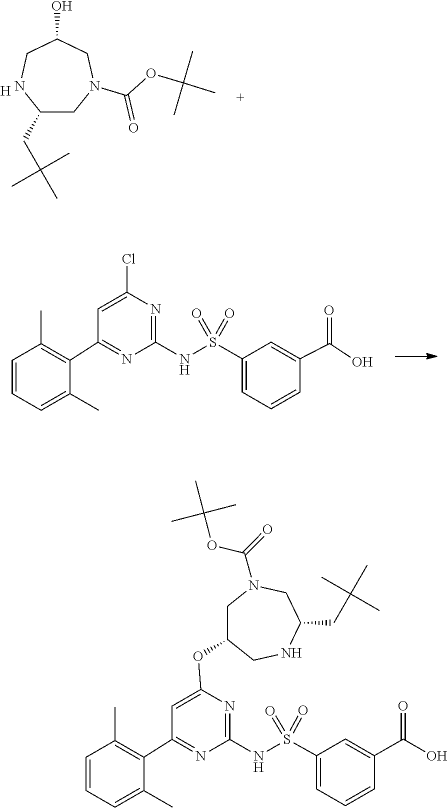

A 20 mL flask was charged under nitrogen with tert-butyl (3S,6S)-3-(2,2-dimethylpropyl)-6-hydroxy-1,4-diazepane-1-carboxylate (244 mg, 0.8519 mmol), 3-[[4-chloro-6-(2,6-dimethylphenyl)pyrimidin-2-yl]sulfamoyl]benzoic acid (364 mg, 0.8711 mmol) and anhydrous THE (3 mL). Sodium tert-butoxide sodium tert-butoxide (327 mg, 3.403 mmol) was added, the vial was capped, and the reaction was stirred at room temperature for 2.5 hours and then at 50° C. for 1.5 hours. The mixture was diluted with aqueous 10% citric acid (20 mL) and the product was extracted with EtOAc (3×20 mL). The combined extracts were dried over sodium sulfate and the solvents were evaporated. The resulting solid was suspended in methanol and the insoluble fraction (mostly starting chloro acid) was filtered out. The filtrate was concentrated and subjected to reverse phase (100 g C₁₈ column) using a gradient of acetonitrile in water containing 5 mM HCl (0 to 20% over 15 min then 20-100% over 20 min). The product eluted around 40-50% MeCN. Evaporation of the pure fractions gave 3-[[4-[[(3S,6S)-1-tert-butoxycarbonyl-3-(2,2-dimethylpropyl)-1,4-diazepan-6-yl]oxy]-6-(2,6-dimethylphenyl)pyrimidin-2-yl]sulfamoyl]benzoic acid (hydrochloride salt) (113 mg, 19%) as an off-white solid. ESI-MS m/z calc. 667.30396, found 668.7 (M+1)⁺; Retention time: 1.36 minutes; LC method A.

### Step 7: tert-Butyl (16S,20S)-12-(2,6-dimethylphenyl)-20-(2,2-dimethylpropyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (Compound 134)

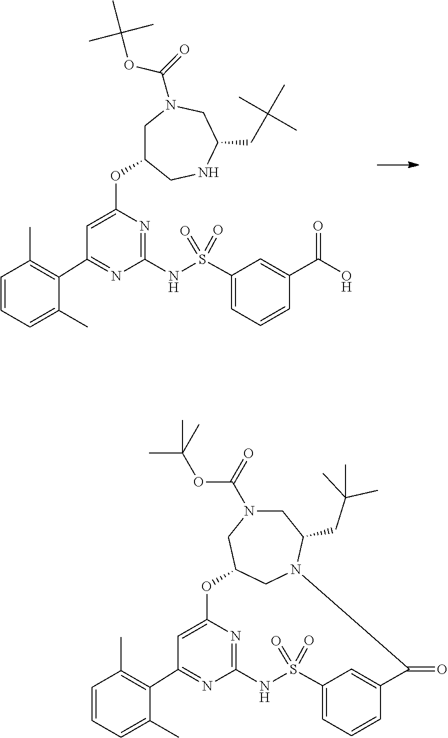 [SMILES: Cc1cccc(C)c1-c1cc(O[C@H]2CN[C@@H](CC(C)(C)C)CN(C(=O)OC(C)(C)C)C2)nc(NS(=O)(=O)c2cccc(C(=O)O)c2)n1 ; Cc1cccc(C)c1-c1cc2nc(n1)NS(=O)(=O)c1cccc(c1)C(=O)N1C[C@@H](CN(C(=O)OC(C)(C)C)C[C@@H]1CC(C)(C)C)O2]

A 100 mL flask was charged under nitrogen with 3-[[4-[[(3S,6S)-1-tert-butoxycarbonyl-3-(2,2-dimethylpropyl)-1,4-diazepan-6-yl]oxy]-6-(2,6-dimethylphenyl)pyrimidin-2-yl]sulfamoyl]benzoic acid (hydrochloride salt) (103 mg, 0.1462 mmol), anhydrous DMF (5 mL), DIEA (100 μL, 0.5741 mmol) and HATU (104 mg, 0.2735 mmol). The mixture was stirred at room temperature for 20 min. The reaction mixture was combined with the small scale reaction (run on 0.01278 mmol) and it was slowly poured into an ice-cold citric acid (20 mL of 10% w/v, 10.41 mmol) aqueous solution under stirring. The resulting solid suspension was extracted with EtOAc (3×40 mL). After drying over sodium sulfate, evaporation of the solvents gave a residue that was dissolved in DCM and purified by flash chromatography on silica gel (12 g column) using a gradient of MeOH (0 to 5% over 30 min) in dichloromethane (two purifications run as the first one eluted early with DMF and impurities). The product eluted around 3-4% MeOH. Evaporation of the solvents and several cycles of trituration/evaporation in DCM/hexanes gave tert-butyl (16S,20S)-12-(2,6-dimethylphenyl)-20-(2,2-dimethylpropyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (60 mg, 57%) as a white solid. ESI-MS m/z calc. 649.2934, found 650.48 (M+1)⁺; Retention time: 2.02 minutes; LC method A.

### Step 8: (16R,20S)-12-(2,6-Dimethylphenyl)-20-(2,2-dimethylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 135)

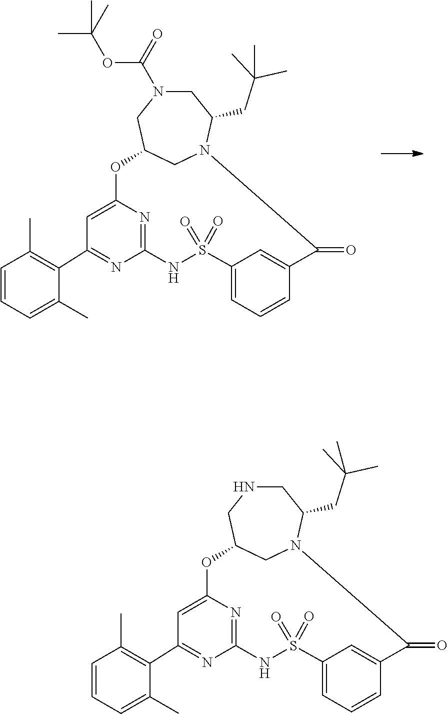 [SMILES: Cc1cccc(C)c1-c1cc2nc(n1)NS(=O)(=O)c1cccc(c1)C(=O)N1C[C@@H](CN(C(=O)OC(C)(C)C)C[C@@H]1CC(C)(C)C)O2 ; Cc1cccc(C)c1-c1cc2nc(n1)NS(=O)(=O)c1cccc(c1)C(=O)N1C[C@@H](CNC[C@@H]1CC(C)(C)C)O2]

A 100 mL flask containing tert-butyl (16S,20S)-12-(2,6-dimethylphenyl)-20-(2,2-dimethylpropyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (57 mg, 0.08772 mmol) was charged under nitrogen with DCM (1 mL). HCl (1 mL of 4 M, 4.000 mmol) (4 M dioxane solution) was added and the mixture was stirred at room temperature for 2 hours (94% conversion). More HCl (500 μL of 4 M, 2.000 mmol) was added and the mixture was stirred for an additional hour. The volatiles were removed by evaporation and the residue was triturated in DCM/hexanes and the solvents were evaporated. The operation was repeated until a solid was obtained. Drying under vacuum gave (16R,20S)-12-(2,6-dimethylphenyl)-20-(2,2-dimethylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (58 mg, 107%) as a white solid. ¹H NMR (500 MHz, DMSO-d₆) δ 8.67 (s, 1H), 7.86 (d, J=7.9 Hz, 1H), 7.65 (t, J=7.8 Hz, 1H), 7.52 (d, J=7.6 Hz, 1H), 7.27 (t, J=7.7 Hz, 1H), 7.12 (d, J=7.7 Hz, 2H), 6.21 (s, 1H), 5.90 (broad s, 1H), 4.83 (br s, 1H), 3.87 (dd, J=14.0, 7.3 Hz, 1H), 3.57 (t, J=13.1 Hz, 1H), 3.47 (dd, J=14.2, 4.8 Hz, 1H), 3.26-3.08 (m, 3H), 2.03 (s, 6H), 1.61-1.44 (m, 2H), 0.98 (s, 9H). ESI-MS m/z calc. 549.24097, found 550.43 (M+1)⁺; Retention time: 1.18 minutes; LC method A.

### Step 9: (16R,20S)-12-(2,6-Dimethylphenyl)-20-(2,2-dimethylpropyl)-18-methyl-15-oxa-8λ⁶-thia-1,9,11,18,22-pentaazatetracyclo[14.4.1.13,7.110,14]tricosa-3(23),4,6,10(22),11,13-hexaene-2,8,8-trione (Compound 136)

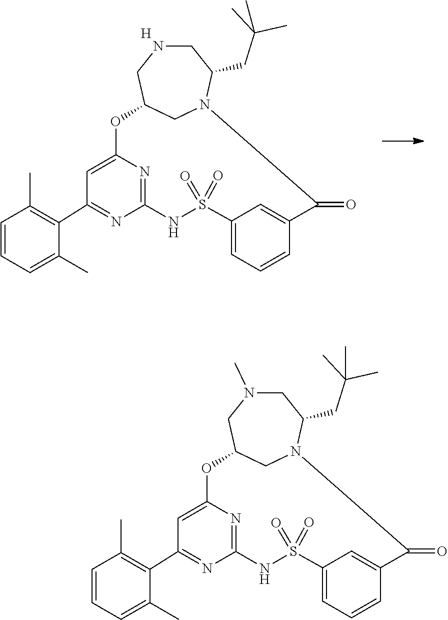

(16R,20S)-12-(2,6-Dimethylphenyl)-20-(2,2-dimethylpropyl)-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (20 mg, 0.03242 mmol) was dissolved in formic acid (250 μL) (88% aqueous) and combined with aqueous formaldehyde (900 μL, 32.67 mmol) (37% aqueous) and heated to 90° C. for 2.5 hours in a screwcap vial. The reaction mixture was then partially concentrated by blowing nitrogen, diluted with DMSO. The solution was microfiltered through syringe filter disc and purified by reverse phase preparative HPLC (Cis) using a gradient of acetonitrile in water (1 to 99% over 15 min) and HCl as a modifier gave (16R,20S)-12-(2,6-dimethylphenyl)-20-(2,2-dimethylpropyl)-18-methyl-15-oxa-8λ⁶-thia-1,9,11,18,22-pentaazatetracyclo[14.4.1.13,7.110,14]tricosa-3(23),4,6,10(22),11,13-hexaene-2,8,8-trione (hydrochloride salt) (3.7 mg, 19%) as an off-white solid. ESI-MS m/z calc. 563.25665, found 564.69 (M+1)⁺; Retention time: 1.16 minutes; LC method A.

### Example 68: Preparation of Compound 137

### Step 1: (3S)-3-Aminotetrahydrofuran-2-one

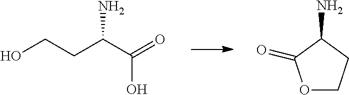 [SMILES: N[C@@H](CCO)C(=O)O ; N[C@H]1CCOC1=O]

(2S)-2-Amino-4-hydroxy-butanoic acid (23.58 g, 197.95 mmol) was dissolved in a solution of aqueous HCl (380 mL of 2.4 M, 912.00 mmol). The solution was refluxed at 140° C. and stirred for 3 hours. The reaction was then cooled to room temperature and stirred overnight. EtOH (5×400 mL) was added to the solution and concentrated until a white solid remained. The solid was cooled on an ice bath and filtered. The white solid was washed with cold ethanol (3×400 mL) and dried to give (3S)-3-aminotetrahydrofuran-2-one (hydrochloride salt) (20.71 g, 75%) as a white powder. ¹H NMR (250 MHz, Deuterium Oxide) δ 4.68-4.52 (m, 1H), 4.52-4.34 (m, 2H), 2.95-2.64 (m, 1H), 2.55-2.26 (m, 1H).

### Step 2: (3S)-3-(Benzylamino)tetrahydrofuran-2-one

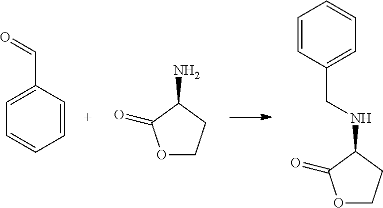 [SMILES: O=Cc1ccccc1 ; N[C@H]1CCOC1=O ; O=C1OCC[C@@H]1NCc1ccccc1]

To a solution of (3S)-3-aminotetrahydrofuran-2-one (hydrochloride salt) (18.36 g, 133.46 mmol) in DCE (275 mL) was added TEA (13.504 g, 18.6 mL, 133.45 mmol) and benzaldehyde (12.792 g, 12.3 mL, 120.54 mmol) and stirred for 10 min at room temperature. Sodium Triacetoxyborohydride (56.6 g, 267.06 mmol) was added at room temperature and the reaction was stirred for 1 hour before being quenched with aqueous sodium bicarbonate (250 mL). The solution was extracted with DCM (3×300 mL). The organic layers were washed with brine (300 mL) then dried over sodium sulfate and concentrated before being purified by silica gel chromatography eluting 0-100% hexanes-diethyl ether to yield (3S)-3-(benzylamino)tetrahydrofuran-2-one (17.93 g, 70%). ¹H NMR (250 MHz, CDCl₃) δ 7.34 (m, 5H), 4.40 (td, J=8.9, 2.1 Hz, 1H), 4.17 (ddd, J=10.5, 9.2, 6.2 Hz, 1H), 3.91 (d, J=1.5 Hz, 2H), 3.55 (dd, J=10.5, 8.1 Hz, 1H), 2.66-2.26 (m, 1H), 2.20-2.08 (m, 1H), 2.04-1.98 (m, 1H). ESI-MS m/z calc. 191.09464, found 192.2 (M+1)⁺; Retention time: 1.89 minutes; LC method T.

### Step 3: (3R,6S)-4-Benzyl-6-hydroxy-3-(2-hydroxyethyl)-1,4-diazepan-2-one

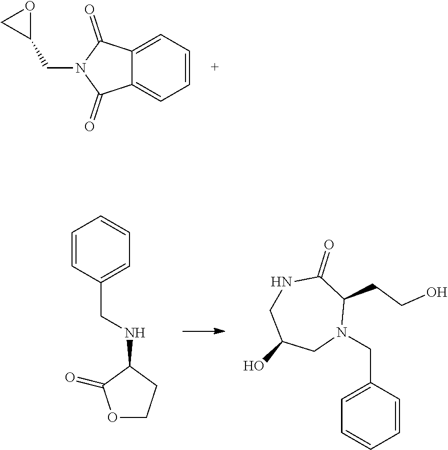 [SMILES: O=C1c2ccccc2C(=O)N1C[C@@H]1CO1 ; O=C1OCC[C@@H]1NCc1ccccc1 ; O=C1NC[C@H](O)CN(Cc2ccccc2)[C@@H]1CCO]

To a solution of 2-[[(2R)-oxiran-2-yl]methyl]isoindoline-1,3-dione (18 g, 88.585 mmol) in ACN (200 mL) was added (3S)-3-(benzylamino)tetrahydrofuran-2-one (16.93 g, 88.534 mmol) and magnesium perchlorate (29.64 g, 132.79 mmol). The reaction mixture was stirred at room temperature for 2 hours before being diluted with water (150 mL) and extracted with DCM (3×150 mL). The combined organic layers were washed with brine (170 mL), dried over anhydrous sodium sulfate and concentrated under vacuum. The organic residue was dissolved in methanol (300 mL) and hydrazine hydrate (8.8638 g, 11.8 mL, 129.26 mmol) was added to the reaction. The reaction was stirred at 65° C. for 24 hours. The reaction was cooled to room temperature and the white solid was filtered off. The filtrate was concentrated and then diluted with 1 N NaOH (40 mL) before being extracted with ethyl acetate (3×40 mL). The combined organic layers were washed with brine (40 mL), dried over sodium sulfate and concentrated before being purified by silica gel chromatography eluting 0-5% DCM-MeOH to give (3R,6S)-4-benzyl-6-hydroxy-3-(2-hydroxyethyl)-1,4-diazepan-2-one (17.06 g, 73%). ¹H NMR (250 MHz, CDCl₃) δ 7.48-7.25 (m, 5H), 6.25 (s, 1H), 4.58-3.67 (m, 5H), 3.48 (s, 2H), 3.40 (d, J=6.6 Hz, 1H), 3.17 (s, 1H), 3.03-2.75 (m, 1H), 2.40-1.71 (m, 2H). ESI-MS m/z calc. 264.1474, found 265.1 (M+1)⁺; Retention time: 2.13 minutes; LC method T.

### Step 4: (2R,6S)-1-Benzyl-2-(2-hydroxyethyl)-1,4-diazepan-6-ol

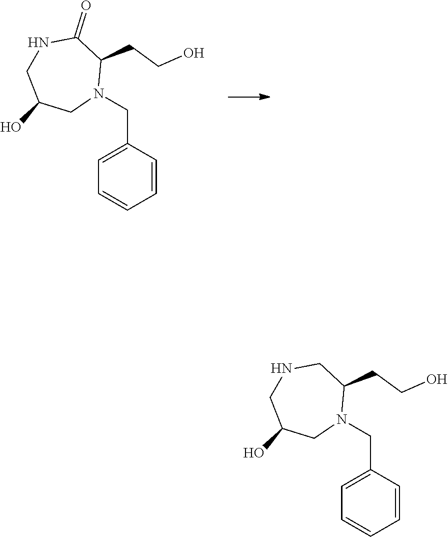 [SMILES: O=C1NC[C@H](O)CN(Cc2ccccc2)[C@@H]1CCO ; OCC[C@@H]1CNC[C@H](O)CN1Cc1ccccc1]

Into a solution of (3R,6S)-4-benzyl-6-hydroxy-3-(2-hydroxyethyl)-1,4-diazepan-2-one (17.06 g, 64.543 mmol) in 2-methyltetrahydrofuran (260 mL) was added LAH (25 g, 658.69 mmol). The reaction was stirred at 45° C. overnight before being cooled to room temperature. The reaction was quenched with water (25 mL), 15% NaOH (aqueous) (25 mL) and water (75 mL) at 0° C. subsequently. The reaction mixture was stirred for another 30 minutes, then it was filtered through a pad of Celite. The filter cake was washed with THE (3×500 mL). The combined filtrate was concentrated under vacuum to give (2R,6S)-1-benzyl-2-(2-hydroxyethyl)-1,4-diazepan-6-ol (13.85 g, 86%). ESI-MS m/z calc. 250.16812, found 251.1 (M+1)⁺; Retention time: 2.03 minutes; LC method T.

### Step 5: tert-Butyl (3R,6R)-4-benzyl-6-hydroxy-3-(2-hydroxyethyl)-1,4-diazepane-1-carboxylate

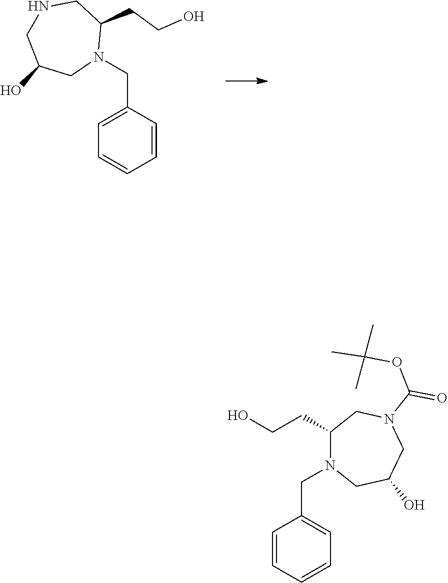 [SMILES: OCC[C@@H]1CNC[C@H](O)CN1Cc1ccccc1 ; CC(C)(C)OC(=O)N1C[C@H](O)CN(Cc2ccccc2)[C@H](CCO)C1]

To a solution of (2R,6S)-1-benzyl-2-(2-hydroxyethyl)-1,4-diazepan-6-ol (13.85 g, 55.325 mmol) in DCM (300 mL) was added Boc anhydride (18.1 g, 82.934 mmol) and triethylamine (8.4216 g, 11.6 mL, 83.226 mmol) at room temperature and stirred overnight. The reaction was quenched with brine (200 mL). The two layers were separated, and the aqueous layer was extracted with DCM (2×500 mL). The combined organic layers were dried over anhydrous magnesium sulfate and concentrated under vacuum. The residue was purified by silica gel chromatography using 0-5% DCM-MeOH to give tert-butyl (3R,6R)-4-benzyl-6-hydroxy-3-(2-hydroxyethyl)-1,4-diazepane-1-carboxylate (11.28 g, 58%). ¹H NMR (250 MHz, CDCl₃) δ 7.30 (d, J=7.1 Hz, 5H), 4.13-3.68 (m, 7H), 3.55-3.24 (m, 3H), 3.19-2.75 (m, 2H), 2.14-1.73 (m, 2H), 1.51 (s, 9H). ESI-MS m/z calc. 350.22055, found 351.2 (M+1)⁺; Retention time: 2.37 minutes; LC method T.

### Step 6: tert-Butyl (3R,6R)-4-benzyl-3-[2-[tert-butyl(dimethyl)silyl]oxyethyl]-6-hydroxy-1,4-diazepane-1-carboxylate

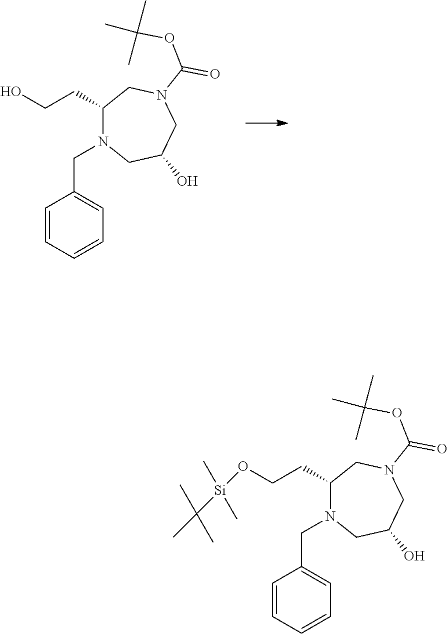 [SMILES: CC(C)(C)OC(=O)N1C[C@H](O)CN(Cc2ccccc2)[C@H](CCO)C1 ; CC(C)(C)OC(=O)N1C[C@H](O)CN(Cc2ccccc2)[C@H](CCO[Si](C)(C)C(C)(C)C)C1]

To a solution of tert-butyl (3R,6R)-4-benzyl-6-hydroxy-3-(2-hydroxyethyl)-1,4-diazepane-1-carboxylate (4.94 g, 14.096 mmol) in THE (100 mL) was added Imidazole (1.9 g, 27.909 mmol) at 0° C. The solution was stirred for 10 min before TBSCl (2.6 g, 17.250 mmol) was added at the same temperature. The reaction was stirred overnight before being quenched with a solution of aqueous ammonium chloride (50 mL) at 0° C. The reaction was extracted with ethyl acetate (3×100 mL) and washed with brine (150 mL) before being dried over sodium sulfate. The organic layer was concentrated in vacuum and purified using silica gel chromatography eluting 10-60% hexanes-diethyl ether to give tert-butyl (3R,6R)-4-benzyl-3-[2-[tert-butyl(dimethyl)silyl]oxyethyl]-6-hydroxy-1,4-diazepane-1-carboxylate (5.383 g, 82%). ¹H NMR (500 MHz, DMSO-d₆) δ 7.60-7.31 (m, 5H), 4.97-4.82 (m, 1H), 4.10-4.02 (m, 1H), 4.00-3.70 (m, 5H), 3.67-3.55 (m, 1H), 3.31 (ddq, J=35.4, 10.3, 5.4, 4.7 Hz, 1H), 3.16-2.72 (m, 3H), 1.84-1.69 (m, 1H), 1.64 (d, J=17.8 Hz, 9H), 1.55 (dt, J=13.4, 6.7 Hz, 1H), 1.12-0.96 (m, 9H), 0.26-0.21 (m, 6H). ESI-MS m/z calc. 464.30704, found 465.3 (M+1)⁺; Retention time: 3.21 minutes; LC method T.

### Step 7: tert-Butyl (3S,6S)-3-[2-[tert-butyl(dimethyl)silyl]oxyethyl]-6-hydroxy-1,4-diazepane-1-carboxylate

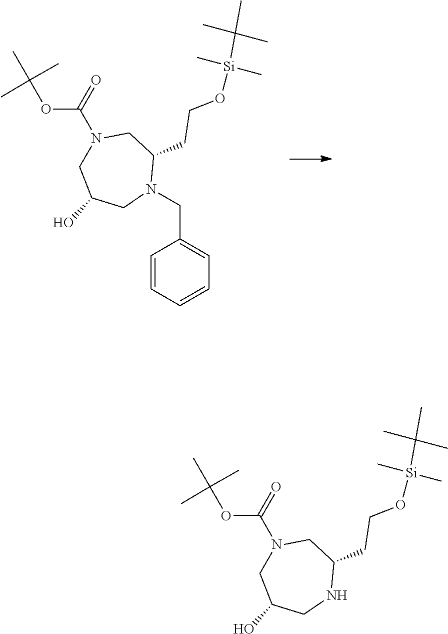 [SMILES: CC(C)(C)OC(=O)N1C[C@@H](O)CN(Cc2ccccc2)[C@@H](CCO[Si](C)(C)C(C)(C)C)C1 ; CC(C)(C)OC(=O)N1C[C@@H](O)CN[C@@H](CCO[Si](C)(C)C(C)(C)C)C1]

A 100 mL round bottom flask was charged with tert-butyl (3S,6S)-4-benzyl-3-[2-[tert-butyl(dimethyl)silyl]oxyethyl]-6-hydroxy-1,4-diazepane-1-carboxylate (1.553 g, 3.342 mmol) and MeOH (30 mL). The solution was sparged with nitrogen for 10 minutes. Pd(OH)₂ (525 mg of 20% w/w, 0.7477 mmol) (20% wet) was added and the reaction was stirred at room temperature under hydrogen (two balloon) for 4 days. The solution was sparged with nitrogen for 10 minutes and filtered through a pad of Celite. After concentration, the solution was microfiltered through a syringe filter disc and the solvent was evaporated to give tert-butyl (3S,6S)-3-[2-[tert-butyl(dimethyl)silyl]oxyethyl]-6-hydroxy-1,4-diazepane-1-carboxylate (1.215 g, 97%) as a colored resin. ESI-MS m/z calc. 374.26007, found 375.75 (M+1)⁺; Retention time: 1.49 minutes; LC method A.

### Step 8: tert-Butyl 3-[2-[tert-butyl(dimethyl)silyl]oxyethyl]-4-[3-[[4-chloro-6-(2,6-dimethylphenyl)pyrimidin-2-yl]sulfamoyl]benzoyl]-6-hydroxy-1,4-diazepane-1-carboxylate, diastereomer 1, and tert-butyl 3-[2-[tert-butyl(dimethyl)silyl]oxyethyl]-4-[3-[[4-chloro-6-(2,6-dimethylphenyl)pyrimidin-2-yl]sulfamoyl]benzoyl]-6-hydroxy-1,4-diazepane-1-carboxylate, diastereomer 2

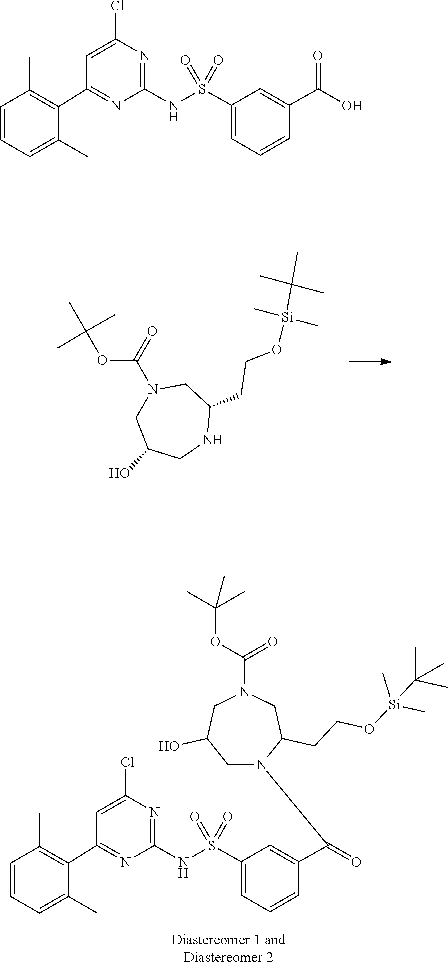 [SMILES: Cc1cccc(C)c1-c1cc(Cl)nc(NS(=O)(=O)c2cccc(C(=O)O)c2)n1 ; CC(C)(C)OC(=O)N1C[C@@H](O)CN[C@@H](CCO[Si](C)(C)C(C)(C)C)C1 ; Cc1cccc(C)c1-c1cc(Cl)nc(NS(=O)(=O)c2cccc(C(=O)N3CC(O)CN(C(=O)OC(C)(C)C)CC3CCO[Si](C)(C)C(C)(C)C)c2)n1]

A 100 mL flask was charged under nitrogen with tert-butyl (3S,6S)-3-[2-[tert-butyl(dimethyl)silyl]oxyethyl]-6-hydroxy-1,4-diazepane-1-carboxylate (1.215 g, 3.244 mmol), anhydrous DMF (18 mL) and 3-[[4-chloro-6-(2,6-dimethylphenyl)pyrimidin-2-yl]sulfamoyl]benzoic acid (1.15 g, 2.752 mmol). After dissolution of the reagents, the mixture was cooled down in an ice bath. DIEA (3.2 mL, 18.37 mmol) and HATU (1.36 g, 3.577 mmol) were added and the mixture was stirred at 0° C. for 30 min then at rt for 5 hours. The reaction was quenched by being poured in citric acid (100 mL of 10% w/v, 52.05 mmol) (10% aqueous) under vigorous stirring and cooled in ice. The resulting white solid was filtered. The solid was dissolved in DCM. After concentration, it was purified by flash chromatography on silica gel (220 g column) using a gradient of methanol (0 to 5% over 30 min) in dichloromethane. Two products with the expected mass eluted as different peak.

The less polar material (diastereomer 1) eluted around 2% MeOH, tert-butyl 3-[2-[tert-butyl(dimethyl)silyl]oxyethyl]-4-[3-[[4-chloro-6-(2,6-dimethylphenyl)pyrimidin-2-yl]sulfamoyl]benzoyl]-6-hydroxy-1,4-diazepane-1-carboxylate (55 mg, 3%). ESI-MS m/z calc. 773.3045, found 774.55 (M+1)⁺; Retention time: 2.38 minutes (LC method A).

The more polar material (diastereomer 2) eluted around 3% MeOH, tert-butyl 3-[2-[tert-butyl(dimethyl)silyl]oxyethyl]-4-[3-[[4-chloro-6-(2,6-dimethylphenyl)pyrimidin-2-yl]sulfamoyl]benzoyl]-6-hydroxy-1,4-diazepane-1-carboxylate (210 mg, 10%). ESI-MS m/z calc. 773.3045, found 774.37 (M+1)⁺; Retention time: 2.34 minutes (LC method A).

### Step 9: tert-butyl 12-(2,6-dimethylphenyl)-20-(2-hydroxyethyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (Compound 137)

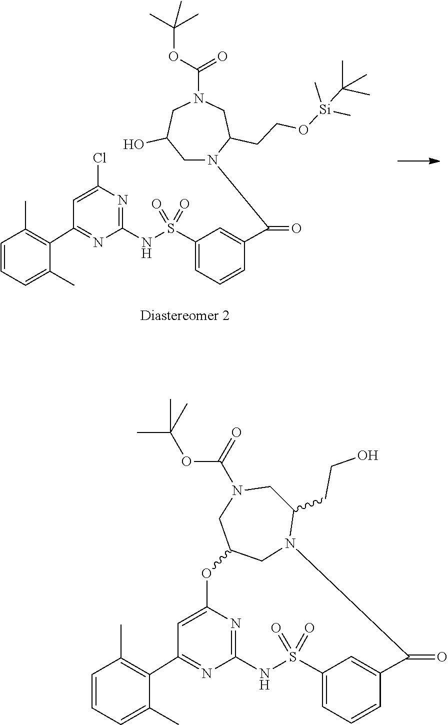 [SMILES: Cc1cccc(C)c1-c1cc(Cl)nc(NS(=O)(=O)c2cccc(C(=O)N3CC(O)CN(C(=O)OC(C)(C)C)CC3CCO[Si](C)(C)C(C)(C)C)c2)n1 ; Cc1cccc(C)c1-c1cc2nc(n1)NS(=O)(=O)c1cccc(c1)C(=O)N1CC(CN(C(=O)OC(C)(C)C)CC1CCO)O2]

A 100 mL flask was charged under nitrogen with tert-butyl 3-[2-[tert-butyl(dimethyl)silyl]oxyethyl]-4-[3-[[4-chloro-6-(2,6-dimethylphenyl)pyrimidin-2-yl]sulfamoyl]benzoyl]-6-hydroxy-1,4-diazepane-1-carboxylate (210 mg, 0.2712 mmol) (major isomer, diastereomer 2) and anhydrous DMF (11 mL). The mixture was cooled down in ice. NaH (88 mg of 60% w/w, 2.200 mmol) (60% mineral oil dispersion) was added in one portion. The mixture was stirred under nitrogen at 0° C. for 10 minutes. The ice bath was removed, and the reaction was vigorously stirred under nitrogen for 2 hours. The reaction mixture was slowly poured into an ice-cold citric acid (40 mL of 10% w/v, 20.82 mmol) 10% aqueous solution under stirring. The resulting solid suspension was extracted with EtOAc (3×75 mL). After drying over sodium sulfate, evaporation of the solvents gave a residue that was dissolved in DCM and purified by flash chromatography on silica gel (24 g column) using a gradient of MeOH (0 to 5% over 30 min) in dichloromethane. The main deprotected product eluted around 4% MeOH. Evaporation of the solvents and several cycle of trituration/evaporation in DCM/hexanes gave tert-butyl 12-(2,6-dimethylphenyl)-20-(2-hydroxyethyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (95 mg, 56%) as a white solid. ESI-MS m/z calc. 623.2414, found 624.3 (M+1)⁺; Retention time: 1.39 minutes (LC method A). A small amount (21 mg) was purified by reverse phase preparative HPLC (Cis) using a gradient of acetonitrile in water (1 to 99% over 15 min) and HCl as a modifier to give tert-butyl 12-(2,6-dimethylphenyl)-20-(2-hydroxyethyl)-2,8,8-trioxo-15-oxa-8λ⁶-thia-1,9,11,18,22-pentaazatetracyclo[14.4.1.13,7.110,14]tricosa-3(23),4,6,10(22),11,13-hexaene-18-carboxylate (15.7 mg, 41%) as a white solid. ESI-MS m/z calc. 623.2414, found 624.3 (M+1)⁺; Retention time: 1.39 minutes (LC method A). A screen of 8 chiral SFC column consistently shows only one peak, suggesting that the product was a single enantiomer of unknown structure.

### Example 69: Characterization of Compounds 138-159

The compounds in the following tables were prepared in a manner analogous to that described above using commercially available reagents and intermediates described herein.

TABLE 7

| Compound | | LCMS Rt | Calc. | | LCMS |
|---|---|---|---|---|---|
| number | Structure | (min) | mass | M + 1 | Method |
| 138 | 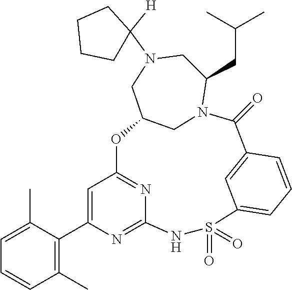 [SMILES: Cc1cccc(C)c1-c1cc2nc(n1)NS(=O)(=O)c1cccc(c1)C(=O)N1C[C@@H](CN(C3CCCC3)C[C@H]1CC(C)C)O2] | 1.32 | 603.288 | 604.4 | A |
| 139 | 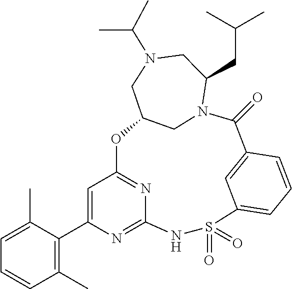 [SMILES: Cc1cccc(C)c1-c1cc2nc(n1)NS(=O)(=O)c1cccc(c1)C(=O)N1C[C@@H](CN(C(C)C)C[C@H]1CC(C)C)O2] | 1.25 | 577.272 | 578.4 | A |
| 140 | 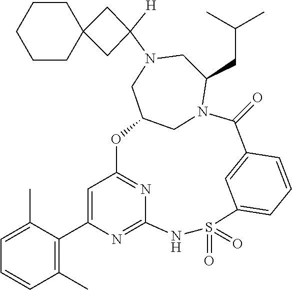 [SMILES: Cc1cccc(C)c1-c1cc2nc(n1)NS(=O)(=O)c1cccc(c1)C(=O)N1C[C@@H](CN(C3CC4(CCCCC4)C3)C[C@H]1CC(C)C)O2] | 1.59 | 657.335 | 658.5 | A |
| 141 | 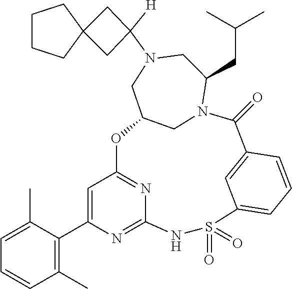 [SMILES: Cc1cccc(C)c1-c1cc2nc(n1)NS(=O)(=O)c1cccc(c1)C(=O)N1C[C@@H](CN(C3CC4(CCCC4)C3)C[C@H]1CC(C)C)O2] | 1.51 | 643.319 | 644.4 | A |
| 142 | 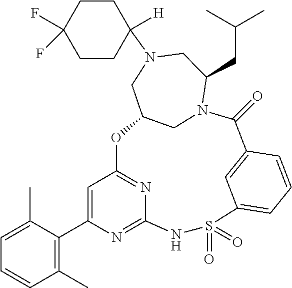 [SMILES: Cc1cccc(C)c1-c1cc2nc(n1)NS(=O)(=O)c1cccc(c1)C(=O)N1C[C@@H](CN(C3CCC(F)(F)CC3)C[C@H]1CC(C)C)O2] | 1.51 | 653.285 | 654.4 | A |
| 143 | 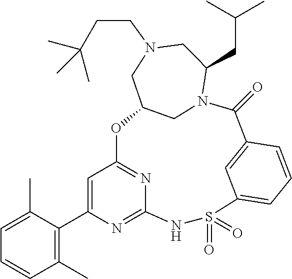 [SMILES: Cc1cccc(C)c1-c1cc2nc(n1)NS(=O)(=O)c1cccc(c1)C(=O)N1C[C@@H](CN(CCC(C)(C)C)C[C@H]1CC(C)C)O2] | 1.5 | 619.319 | 620.4 | A |
| 144 | 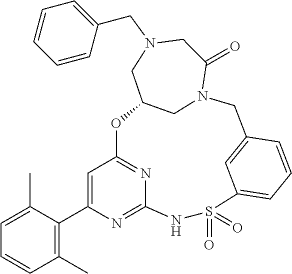 [SMILES: Cc1cccc(C)c1-c1cc2nc(n1)NS(=O)(=O)c1cccc(c1)CN1C[C@@H](CN(Cc3ccccc3)CC1=O)O2] | 1.57 | 569.21 | 570.3 | A |
| 145 | 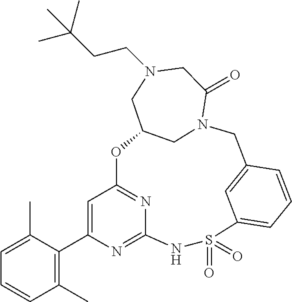 [SMILES: Cc1cccc(C)c1-c1cc2nc(n1)NS(=O)(=O)c1cccc(c1)CN1C[C@@H](CN(CCC(C)(C)C)CC1=O)O2] | 1.42 | 563.257 | 564.4 | A |
| 146 | 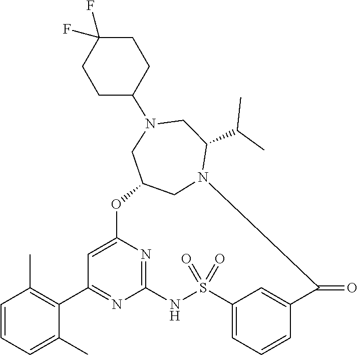 [SMILES: Cc1cccc(C)c1-c1cc2nc(n1)NS(=O)(=O)c1cccc(c1)C(=O)N1C[C@@H](CN(C3CCC(F)(F)CC3)C[C@@H]1C(C)C)O2] | 1.4 | 639.269 | 640.55 | A |
| 147 | 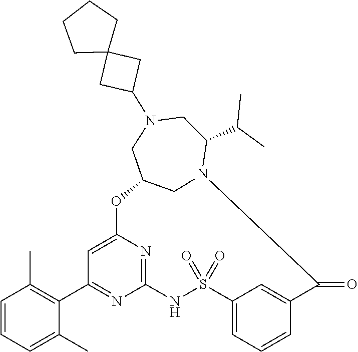 [SMILES: Cc1cccc(C)c1-c1cc2nc(n1)NS(=O)(=O)c1cccc(c1)C(=O)N1C[C@@H](CN(C3CC4(CCCC4)C3)C[C@@H]1C(C)C)O2] | 1.4 | 629.304 | 630.65 | A |
| 148 | 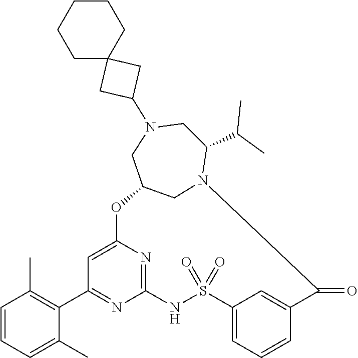 [SMILES: Cc1cccc(C)c1-c1cc2nc(n1)NS(=O)(=O)c1cccc(c1)C(=O)N1C[C@@H](CN(C3CC4(CCCCC4)C3)C[C@@H]1C(C)C)O2] | 1.47 | 643.319 | 644.82 | A |
| 149 | 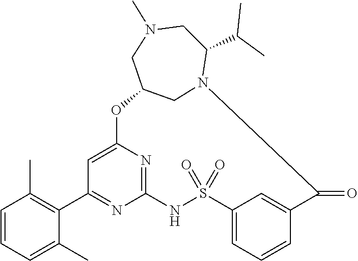 [SMILES: Cc1cccc(C)c1-c1cc2nc(n1)NS(=O)(=O)c1cccc(c1)C(=O)N1C[C@@H](CN(C)C[C@@H]1C(C)C)O2] | 1 | 535.225 | 536.49 | A |
| 150 | 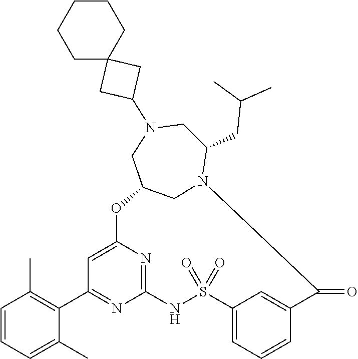 [SMILES: Cc1cccc(C)c1-c1cc2nc(n1)NS(=O)(=O)c1cccc(c1)C(=O)N1C[C@@H](CN(C3CC4(CCCCC4)C3)C[C@@H]1CC(C)C)O2] | 1.51 | 657.335 | 658.7 | A |
| 151 | 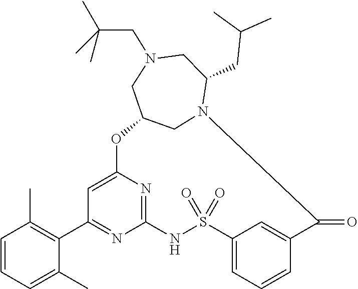 [SMILES: Cc1cccc(C)c1-c1cc2nc(n1)NS(=O)(=O)c1cccc(c1)C(=O)N1C[C@@H](CN(CC(C)(C)C)C[C@@H]1CC(C)C)O2] | 1.8 | 605.304 | 606.74 | A |
| 152 | 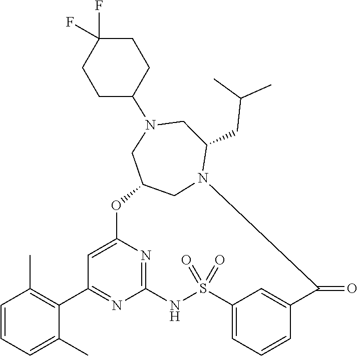 [SMILES: Cc1cccc(C)c1-c1cc2nc(n1)NS(=O)(=O)c1cccc(c1)C(=O)N1C[C@@H](CN(C3CCC(F)(F)CC3)C[C@@H]1CC(C)C)O2] | 1.47 | 653.285 | 654.53 | A |
| 153 | 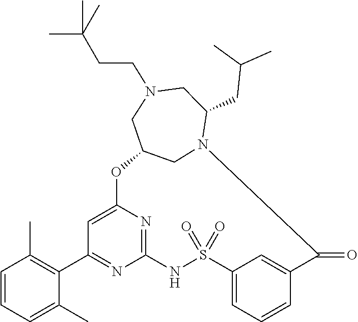 [SMILES: Cc1cccc(C)c1-c1cc2nc(n1)NS(=O)(=O)c1cccc(c1)C(=O)N1C[C@@H](CN(CCC(C)(C)C)C[C@@H]1CC(C)C)O2] | 1.39 | 619.319 | 620.8 | A |
| 154 | 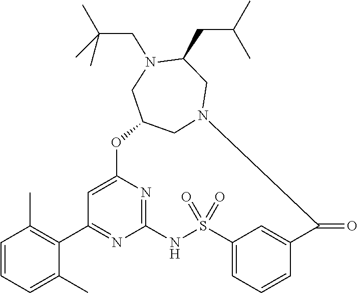 [SMILES: Cc1cccc(C)c1-c1cc2nc(n1)NS(=O)(=O)c1cccc(c1)C(=O)N1C[C@@H](CN(CC(C)(C)C)[C@@H](CC(C)C)C1)O2] | 1.75 | 605.304 | 606.37 | A |
| 155 | 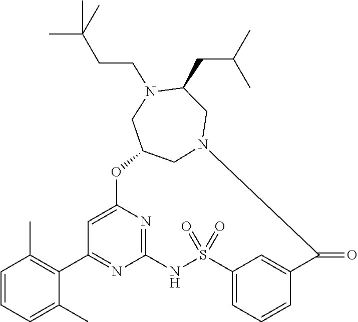 [SMILES: Cc1cccc(C)c1-c1cc2nc(n1)NS(=O)(=O)c1cccc(c1)C(=O)N1C[C@@H](CN(CCC(C)(C)C)[C@@H](CC(C)C)C1)O2] | 1.38 | 619.319 | 620.49 | A |
| 156 | 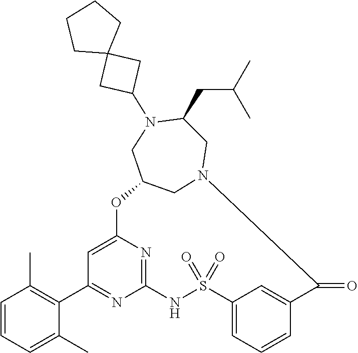 [SMILES: Cc1cccc(C)c1-c1cc2nc(n1)NS(=O)(=O)c1cccc(c1)C(=O)N1C[C@@H](CN(C3CC4(CCCC4)C3)[C@@H](CC(C)C)C1)O2] | 1.41 | 643.319 | 644.43 | A |
| 157 | 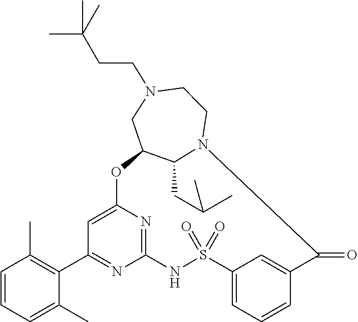 [SMILES: Cc1cccc(C)c1-c1cc2nc(n1)NS(=O)(=O)c1cccc(c1)C(=O)N1CCN(CCC(C)(C)C)C[C@H](O2)[C@H]1CC(C)C] | 1.3 | 619.319 | 620.52 | A |
| 158 | 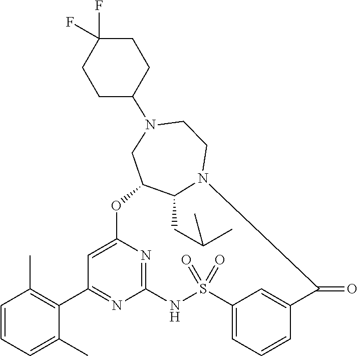 [SMILES: Cc1cccc(C)c1-c1cc2nc(n1)NS(=O)(=O)c1cccc(c1)C(=O)N1CCN(C3CCC(F)(F)CC3)C[C@@H](O2)[C@H]1CC(C)C] | 1.3 | 653.285 | 654.65 | A |
| 159 | 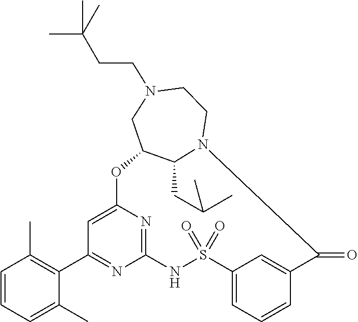 [SMILES: Cc1cccc(C)c1-c1cc2nc(n1)NS(=O)(=O)c1cccc(c1)C(=O)N1CCN(CCC(C)(C)C)C[C@@H](O2)[C@H]1CC(C)C] | 1.36 | 619.319 | 620.52 | A |

TABLE 8

| Com- | |
|---|---|
| pound | |
| number | NMR |
| 138 | ¹H NMR (499 MHz, DMSO-d₆) δ 10.65 (s, 1H), 8.89 (s, 1H), 7.91 (d, J = |
| | 7.7 Hz, 1H), 7.66 (s, 1H), 7.51 (s, 1H), 7.25 (t, J = 8.5 Hz, 1H), 7.13 (d, J = |
| | 7.6 Hz, 2H), 6.33 (s, 1H), 5.76-5.60 (m, 1H), 4.28 (q, J = 11.1 Hz, 1H), |
| | 4.09-3.93 (m, 2H), 3.76 (td, J = 12.3, 5.2 Hz, 1H), 3.72-3.62 (m, 2H), |
| | 3.00 (dd, J = 14.0, 10.6 Hz, 1H), 2.59-2.51 (m, 2H), 2.19-2.10 (m, 2H), |
| | 2.03 (s, 6H), 1.92-1.81 (m, 2H), 1.81-1.73 (m, 2H), 1.69 (p, J = 6.3 Hz, |
| | 1H), 1.64-1.53 (m, 2H), 1.38-1.29 (m, 1H), 0.97 (d, J = 6.6 Hz, 3H), |
| | 0.91 (d, J = 6.6 Hz, 3H). |
| 139 | ¹H NMR (499 MHz, DMSO-d₆) δ 10.44 (s, 1H), 8.91 (s, 1H), 7.90 (s, 1H), |
| | 7.66 (s, 1H), 7.51 (s, 1H), 7.26 (t, J = 7.7 Hz, 1H), 7.13 (d, J = 7.7 Hz, 2H), |
| | 6.35 (s, 1H), 5.78-5.66 (m, 1H), 4.28-4.14 (m, 1H), 4.07-3.95 (m, 1H), |
| | 3.93-3.82 (m, 1H), 3.72 (d, J = 11.9 Hz, 1H), 3.66 (dd, J = 14.6, 5.0 Hz, |
| | 1H), 3.61 (dd, J = 12.1, 5.2 Hz, 1H), 2.97 (dd, J = 13.9, 10.6 Hz, 1H), 2.59- |
| | 2.51 (m, 2H), 2.04 (s, 6H), 1.74-1.64 (m, 1H), 1.38 (d, J = 6.5 Hz, 3H), |
| | 1.36 (d, J = 6.6 Hz, 3H), 1.33 (dd, J = 9.2, 4.3 Hz, 1H), 0.97 (d, J = 6.6 |
| | Hz, 3H), 0.91 (d, J = 6.6 Hz, 3H). |
| 140 | ¹H NMR (499 MHz, DMSO-d₆) δ 10.98 (s, 1H), 8.94 (s, 1H), 7.90 (s, 1H), |
| | 7.65 (s, 1H), 7.51 (s, 1H), 7.26 (t, J = 7.6 Hz, 1H), 7.12 (d, J = 7.6 Hz, 2H), |
| | 6.33 (s, 1H), 5.83-5.65 (m, 1H), 4.13 (q, J = 11.1 Hz, 1H), 3.99 (p, J = |
| | 8.6 Hz, 2H), 3.79 (td, J = 12.3, 5.2 Hz, 1H), 3.64 (dd, J = 14.2, 4.9 Hz, |
| | 1H), 3.55-3.46 (m, 1H), 3.17-3.11 (m, 1H), 3.07 (dd, J = 14.0, 10.7 Hz, |
| | 1H), 2.28-2.22 (m, 1H), 2.20-2.15 (m, 1H), 2.14-2.09 (m, 2H), 2.04 (s, |
| | 6H), 1.68 (dp, J = 13.2, 6.6 Hz, 1H), 1.54-1.45 (m, 4H), 1.44-1.28 (m, |
| | 8H), 0.96 (d, J = 6.5 Hz, 3H), 0.91 (d, J = 6.5 Hz, 3H). |
| 141 | ¹H NMR (499 MHz, DMSO-d₆) δ 10.68 (s, 1H), 8.89 (s, 1H), 7.90 (s, 1H), |
| | 7.66 (s, 1H), 7.52 (s, 1H), 7.25 (t, J = 8.7 Hz, 1H), 7.12 (d, J = 7.6 Hz, 2H), |
| | 6.32 (s, 1H), 5.78-5.60 (m, 1H), 4.14 (q, J = 11.1 Hz, 1H), 4.00 (q, J = |
| | 8.1 Hz, 2H), 3.76 (dt, J = 12.2, 7.2 Hz, 1H), 3.63 (dd, J = 13.9, 4.8 Hz, |
| | 1H), 3.51 (d, J = 13.0 Hz, 1H), 3.23-3.14 (m, 1H), 3.12-3.04 (m, 1H), |
| | 2.31-2.22 (m, 3H), 2.03 (s, 7H), 1.66 (t, J = 6.9 Hz, 3H), 1.62-1.51 (m, |
| | 7H), 1.39-1.28 (m, 1H), 0.96 (d, J = 6.6 Hz, 3H), 0.91 (d, J = 6.6 Hz, 3H). |
| 142 | ¹H NMR (499 MHz, DMSO-d₆) δ 10.87 (s, 1H), 8.92 (s, 1H), 7.90 (s, 1H), |
| | 7.65 (s, 1H), 7.51 (s, 1H), 7.26 (t, J = 7.6 Hz, 1H), 7.13 (d, J = 7.7 Hz, 2H), |
| | 6.34 (s, 1H), 5.81-5.66 (m, 1H), 4.28 (q, J = 11.5 Hz, 1H), 4.08-3.94 (m, |
| | 1H), 3.83-3.70 (m, 2H), 3.69-3.59 (m, 2H), 3.52-3.40 (m, 2H), 3.04- |
| | 2.93 (m, 1H), 2.32-2.13 (m, 4H), 2.04 (s, 7H), 1.98-1.87 (m, 2H), 1.82- |
| | 1.74 (m, 1H), 1.73-1.65 (m, 1H), 1.44-1.31 (m, 1H), 0.97 (d, J = 6.6 Hz, |
| | 3H), 0.91 (d, J = 6.6 Hz, 3H). |
| 143 | ¹H NMR (499 MHz, DMSO-d₆) δ 13.05 (s, 1H), 10.55 (s, 1H), 8.90 (s, 1H), |
| | 7.90 (s, 1H), 7.66 (s, 1H), 7.51 (s, 1H), 7.25 (d, J = 8.6 Hz, 1H), 7.13 (d, J = |
| | 7.7 Hz, 2H), 6.36 (s, 1H), 5.69 (s, 1H), 4.25 (q, J = 11.1 Hz, 1H), 4.03- |
| | 3.92 (m, 1H), 3.85-3.74 (m, 2H), 3.60 (dd, J = 14.1, 4.8 Hz, 1H), 3.44- |
| | 3.36 (m, 2H), 3.05 (dd, J = 14.0, 10.7 Hz, 1H), 2.55-2.52 (m, 1H), 2.03 |
| | (s, 6H), 1.77-1.65 (m, 3H), 1.38-1.30 (m, 1H), 0.97 (d, J = 6.6 Hz, 3H), |
| | 0.96 (s, 9H), 0.92 (d, J = 6.6 Hz, 3H). |
| 144 | ¹H NMR (400 MHZ, Methanol-d₄) δ 8.32 (s, 1H), 7.83 (dt, J = 7.1, 2.0 Hz, |
| | 1H), 7.63-7.54 (m, 4H), 7.54-7.45 (m, 3H), 7.28-7.21 (m, 1H), 7.11 (d, |
| | J = 7.6 Hz, 2H), 6.19 (s, 1H), 6.14-6.03 (m, 1H), 5.78 (d, J = 16.3 Hz, |
| | 1H), 4.44 (q, J = 13.1 Hz, 2H), 4.36 (d, J = 14.8 Hz, 1H), 4.06 (t, J = 16.7 |
| | Hz, 2H), 3.88 (dd, J = 14.8, 10.3 Hz, 1H), 3.68 (d, J = 12.5 Hz, 1H), 3.50- |
| | 3.39 (m, 1H), 3.27-3.19 (m, 1H), 2.06 (s, 6H). |
| 145 | ¹H NMR (400 MHZ, Methanol-d₄) δ 8.28 (s, 1H), 7.84 (dt, J = 6.7, 2.1 Hz, |
| | 1H), 7.63-7.53 (m, 2H), 7.25 (t, J = 7.6 Hz, 1H), 7.12 (d, J = 7.6 Hz, 2H), |
| | 6.24 (s, 1H), 6.13 (tq, J = 9.2, 2.6 Hz, 1H), 5.79 (d, J = 16.2 Hz, 1H), 4.59 |
| | (d, J = 14.6 Hz, 1H), 4.17 (dd, J = 14.6, 1.5 Hz, 1H), 4.11 (d, J = 16.2 Hz, |
| | 1H), 4.04-3.86 (m, 2H), 3.68 (t, J = 11.9 Hz, 1H), 3.55-3.40 (m, 2H), |
| | 3.30-3.25 (m, 1H), 2.07 (s, 6H), 1.87-1.71 (m, 2H), 1.03 (s, 9H) |
| 146 | ¹H NMR (500 MHZ, DMSO-d₆ + 10% D₂O) δ 8.59 (s, 1H), 7.86 (d, J = 7.8 |
| | Hz, 1H), 7.66 (t, J = 7.6 Hz, 1H), 7.53 (d, J = 7.6 Hz, 1H), 7.27 (t, J = 7.7 |
| | Hz, 1H), 7.13 (d, J = 7.7 Hz, 2H), 6.17 (s, 1H), 5.79 (broad s, 1H), 4.58 (br |
| | s, 1H), 3.87-3.55 (m, 3H overlapped with water), 3.53-3.21 (m, 3H), 2.30- |
| | 1.66 (m, 16H), 0.95 (dd, J = 15.0, 6.8 Hz, 6H). |
| 150 | ¹H NMR (499 MHz, DMSO-d₆ + 10% D₂O) δ 8.54 (s, 1H), 7.86 (d, J = 7.2 |
| | Hz, 1H), 7.65 (td, J = 7.8, 1.5 Hz, 1H), 7.55 (d, J = 8.5 Hz, 1H), 7.25 (t, J = |
| | 7.6 Hz, 1H), 7.11 (d, J = 7.6 Hz, 2H), 6.15 (s, 1H), 5.79 (broad s, 1H), |
| | 4.72 (br s, 1H), 3.81 (br s, 1H), 3.69 (br s, 1H), 3.54 (t, J = 13.2 Hz, 1H), |
| | 3.45 (br s, 1H), 3.36-3.17 (m, 2H), 3.09 (br s, 1H), 2.28 (br s, 1H), 2.14 |
| | (br s, 1H), 2.00 (br s, 6H), 1.92-1.75 (m, 2H), 1.62-1.52 (m, 1H), 1.49- |
| | 1.25 (m, 12H), 0.92 (d, J = 6.5 Hz, 6H). |

### Example 70: Preparation of Compound 160

### Step 1: tert-Butyl 3-[2-chloro-6-(2,6-dimethylphenyl)pyrimidin-4-yl]oxypiperidine-1-carboxylate

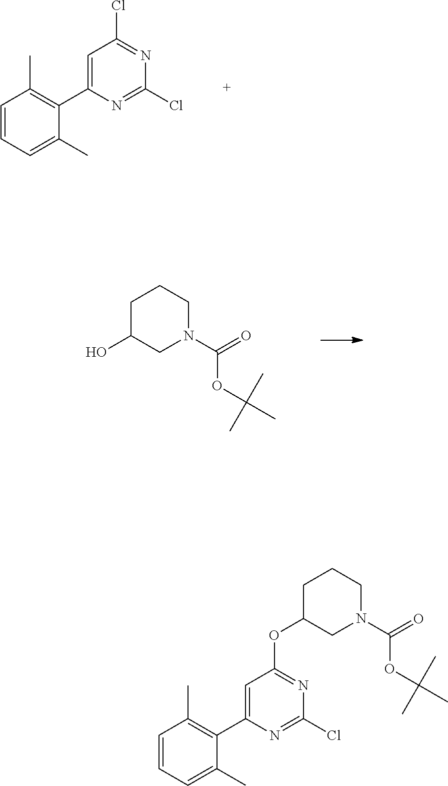 [SMILES: Cc1cccc(C)c1-c1cc(Cl)nc(Cl)n1 ; CC(C)(C)OC(=O)N1CCCC(O)C1 ; Cc1cccc(C)c1-c1cc(OC2CCCN(C(=O)OC(C)(C)C)C2)nc(Cl)n1]

A solution of tert-butyl 3-hydroxypiperidine-1-carboxylate (approximately 1.209 g, 6.005 mmol) in NMP (20 mL) was cooled in an ice bath, and sodium hydride (0.24 g of 60% w/w, 6.001 mmol) was added. After 90 minutes a solution of 2,4-dichloro-6-(2,6-dimethylphenyl)pyrimidine (1.52 g, 6.005 mmol) in NMP (10 mL) was added. The reaction was allowed to slowly warm to room temperature and was stirred for three days. It was quenched with saturated aqueous ammonium chloride, diluted with water, and extracted with ethyl acetate. The combined extracts were washed with water, dried over sodium sulfate, and evaporated. The residue was purified by silica gel column chromatography with 0-30% ethyl acetate in hexanes to give tert-butyl 3-[2-chloro-6-(2,6-dimethylphenyl)pyrimidin-4-yl]oxypiperidine-1-carboxylate (1.36 g, 54%) as a colorless solid. ESI-MS m/z calc. 417.18192, found 418.2 (M+1)⁺; Retention time: 0.86 minutes; LC method D.

### Step 2: 2-Chloro-4-(2,6-dimethylphenyl)-6-(3-piperidyloxy)pyrimidine

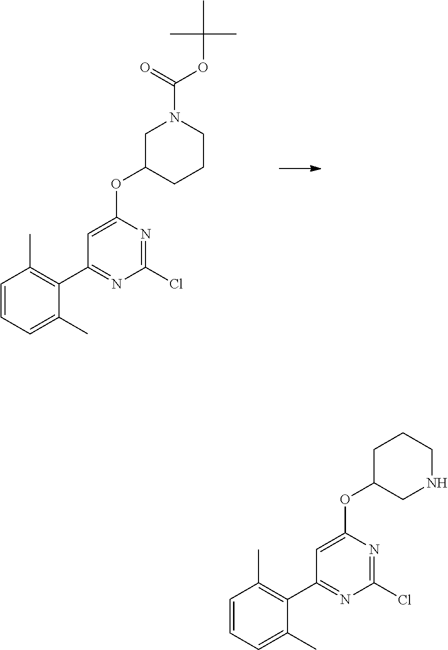 [SMILES: Cc1cccc(C)c1-c1cc(OC2CCCN(C(=O)OC(C)(C)C)C2)nc(Cl)n1 ; Cc1cccc(C)c1-c1cc(OC2CCCNC2)nc(Cl)n1]

A solution of tert-butyl 3-[2-chloro-6-(2,6-dimethylphenyl)pyrimidin-4-yl]oxypiperidine-1-carboxylate (1.36 g, 3.254 mmol) in HCl (15 mL of 4 M, 60.00 mmol) (in dioxane) was stirred for two hours. The solvent was removed under vacuum, the residue was suspended in acetonitrile, and the solvent was again removed under vacuum. The resulting solid was triturated with diethyl ether and dried under vacuum to give a light tan solid, 2-chloro-4-(2,6-dimethylphenyl)-6-(3-piperidyloxy)pyrimidine (hydrochloride salt) (1.15 g, 100%) ESI-MS m/z calc. 317.1295, found 318.1 (M+1)⁺; Retention time: 0.5 minutes; LC method D.

### Step 3: 2-[3-[2-Chloro-6-(2,6-dimethylphenyl)pyrimidin-4-yl]oxy-1-piperidyl]-N-(3-sulfamoylphenyl)acetamide

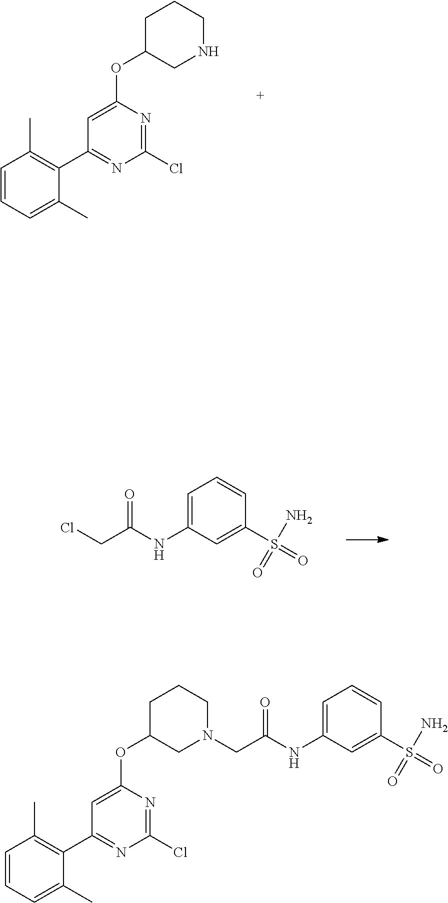 [SMILES: Cc1cccc(C)c1-c1cc(OC2CCCNC2)nc(Cl)n1 ; NS(=O)(=O)c1cccc(NC(=O)CCl)c1 ; Cc1cccc(C)c1-c1cc(OC2CCCN(CC(=O)Nc3cccc(S(N)(=O)=O)c3)C2)nc(Cl)n1]

A solution of 2-chloro-4-(2,6-dimethylphenyl)-6-(3-piperidyloxy)pyrimidine (hydrochloride salt) (0.14 g, 0.3952 mmol), 2-chloro-N-(3-sulfamoylphenyl)acetamide (0.11 g, 0.4423 mmol), and sodium bicarbonate (0.17 g, 2.024 mmol) in NMP (2 mL) was stirred for three days. The reaction was diluted with water and extracted with ethyl acetate. The combined extracts were washed with water, dried over sodium sulfate, and evaporated. The residue was purified by silica gel column chromatography with 0-6% methanol in dichloromethane to give a colorless solid, 2-[3-[2-chloro-6-(2,6-dimethylphenyl)pyrimidin-4-yl]oxy-1-piperidyl]-N-(3-sulfamoylphenyl)acetamide (0.16 g, 76%) ESI-MS m/z calc. 529.155, found 530.3 (M+1)⁺; Retention time: 0.47 minutes; LC method D.

### Step 4: 5-(2,6-Dimethylphenyl)-2-oxa-9λ⁶-thia-6,8,15,18,24-pentaazatetracyclo[16.3.1.13,7.110,14]tetracosa-3(24),4,6,10,12,14(23)-hexaene-9,9,16-trione (Compound 160)

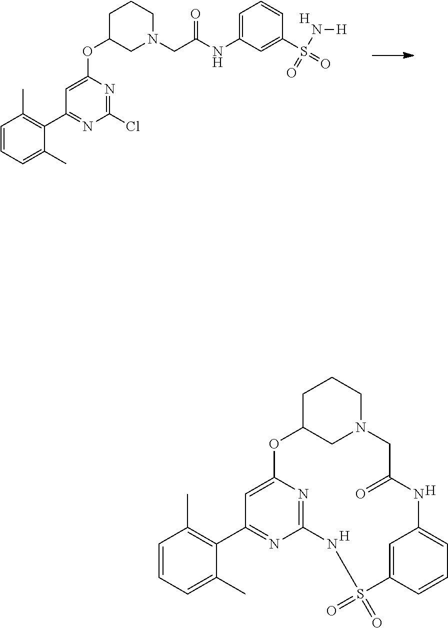

A mixture of 2-[3-[2-chloro-6-(2,6-dimethylphenyl)pyrimidin-4-yl]oxy-1-piperidyl]-N-(3-sulfamoylphenyl)acetamide (0.16 g, 0.3019 mmol), Palladium (II) acetate (14 mg, 0.06236 mmol), 4,5-bis(diphenylphosphino)-9,9-dimethylxanthene (36 mg, 0.06222 mmol), and cesium carbonate (0.20 g, 0.6138 mmol) in dioxane (15 mL) was degassed with a stream of nitrogen and stirred at 100° C. for 16 hours. The reaction was filtered and purified using reverse-phase HPLC to give a colorless solid, 5-(2,6-dimethylphenyl)-2-oxa-9λ⁶-thia-6,8,15,18,24-pentaazatetracyclo[16.3.1.13,7.110,14]tetracosa-3(24),4,6,10,12,14(23)-hexaene-9,9,16-trione (hydrochloride salt) (27 mg, 17%) ESI-MS m/z calc. 493.17838, found 494.3 (M+1)⁺; Retention time: 1.16 minutes; LC method A.

### Example 71: Preparation of Compound 161

### Step 1: tert-Butyl (3R)-4-benzyl-3-(hydroxymethyl)piperazine-1-carboxylate

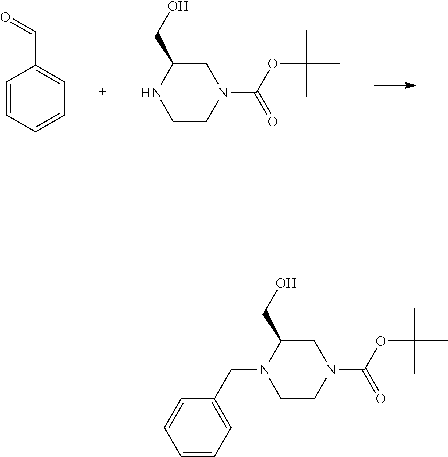 [SMILES: O=Cc1ccccc1 ; CC(C)(C)OC(=O)N1CCN[C@@H](CO)C1 ; CC(C)(C)OC(=O)N1CCN(Cc2ccccc2)[C@@H](CO)C1]

tert-Butyl (3R)-3-(hydroxymethyl)piperazine-1-carboxylate (3 g, 13.87 mmol) and benzaldehyde (2.5 mL, 24.59 mmol) were combined in anhydrous DCE (40 mL) with acetic acid (1.45 mL, 25.50 mmol), and stirred for 30 minutes at room temperature. The reaction mixture was then cooled to 0° C., and sodium triacetoxyborohydride (6.2 g, 29.25 mmol) was added. The reaction mixture was returned to room temperature and stirred for 16 hours. The reaction mixture was poured into aqueous sodium bicarbonate and extracted 3× ethyl acetate. The combined organic layers were washed with brine, dried over sodium sulfate, then concentrated and dried for 72 hours on high vac to give as a white solid, tert-butyl (3R)-4-benzyl-3-(hydroxymethyl)piperazine-1-carboxylate (4.15 g, 98%). ¹H NMR (400 MHz, DMSO) δ 7.31 (d, J 4.3 Hz, 4H), 2.63-2.54 (m, 1H), 7.29-7.20 (m, 1H), 4.61 (t, J=5.2 Hz, 1H), 3.99 (d, J=13.7 Hz, 1H), 3.79-3.66 (m, 2H), 3.48 (d, J=12.7 Hz, 1H), 3.36 (dd, J=5.6, 1.4 Hz, 1H), 3.00 (td, J=9.3, 4.6 Hz, 1H), 2.32 (dt, J=8.0, 3.8 Hz, 1H), 2.13-1.99 (m, 1H), 1.39 (s, 9H). ESI-MS m/z calc. 306.19434, found 307.3 (M+1)⁺; Retention time: 0.36 minutes; LC method D.

### Step 2: 3-[[4-[[(2R)-1-Benzyl-4-tert-butoxycarbonyl-piperazin-2-yl]methoxy]-6-(2,6-dimethylphenyl)pyrimidin-2-yl]sulfamoyl]benzoic acid

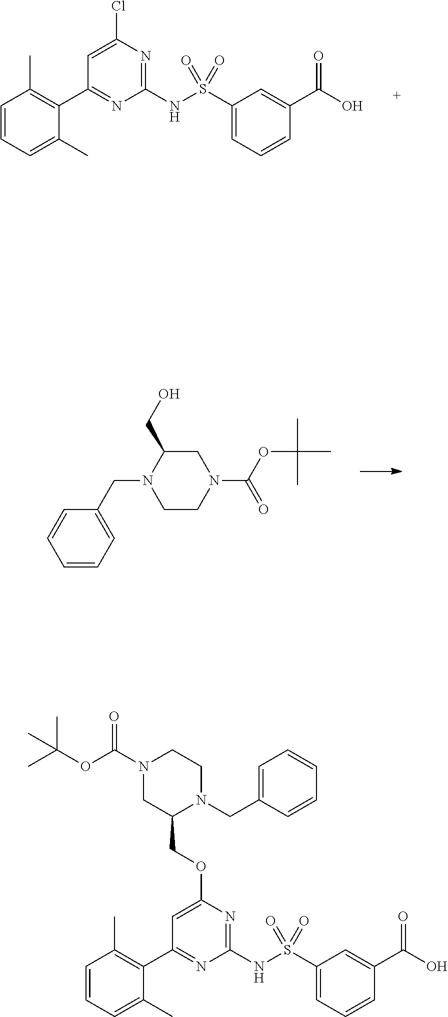

3-[[4-Chloro-6-(2,6-dimethylphenyl)pyrimidin-2-yl]sulfamoyl]benzoic acid (4.062 g, 9.721 mmol) and tert-butyl (3R)-4-benzyl-3-(hydroxymethyl)piperazine-1-carboxylate (3.614 g, 11.80 mmol) were combined in anhydrous NMP (50 mL). The reaction mixture was cooled to 0° C., and NaH (1.75 g, 43.75 mmol) was slowly added in portions under a stream of nitrogen. The reaction mixture was then stirred at room temperature for 1 hour. After this time, the reaction mixture was slowly added to a flask, cooled in an ice/water bath containing aqueous ammonium chloride and ethyl acetate. The aqueous and organic layers were separated, and the aqueous was extracted an additional 4× ethyl acetate. The aqueous layer was further acidified to pH=6 with 1 M HCl and extracted an additional 2× ethyl acetate. The combined organics were washed 3× with water. The aqueous layer from these washings was found to have a pH of 8, and additional aqueous ammonium chloride and 1 M HCl were added until a pH of 6 was reached. The acidified aqueous layer was then extracted 3× ethyl acetate, and these separate organics were washed with brine, dried over sodium sulfate, and concentrated to give 3-[[4-[[(2R)-1-benzyl-4-tert-butoxycarbonyl-piperazin-2-yl]methoxy]-6-(2,6-dimethylphenyl)pyrimidin-2-yl]sulfamoyl]benzoic acid (4.34 g, 65%) ESI-MS m/z calc. 687.27264, found 688.4 (M+1)⁺; Retention time: 0.54 minutes, which was used in the next step without further purification. The original organic layer was separately washed with brine, dried over sodium sulfate and concentrated. The resulting crude material was purified by chromatography on silica gel, eluting with 0-10% methanol in dichloromethane. Fractions containing product were combined and concentrated to give 3-[[4-[[(2R)-1-benzyl-4-tert-butoxycarbonyl-piperazin-2-yl]methoxy]-6-(2,6-dimethylphenyl)pyrimidin-2-yl]sulfamoyl]benzoic acid (1.6 g, 24%). ESI-MS m/z calc. 687.27264, found 688.5 (M+1)⁺; Retention time: 0.54 minutes, LC method D.

### Step 3: (8R)-13-(2,6-Dimethylphenyl)-10-oxa-17λ⁶-thia-3,6,14,16,23-pentaazatetracyclo[16.3.1.111,15.03,8]tricosa-1(21),11,13,15(23),18(22),19-hexaene-2,17,17-trione (Compound 161)

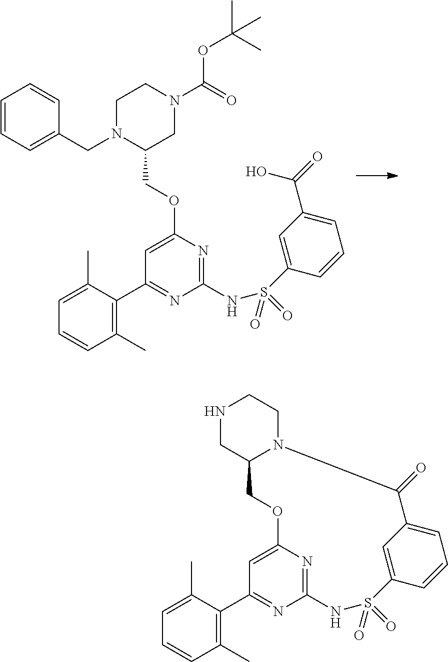 [SMILES: Cc1cccc(C)c1-c1cc(OC[C@H]2CN(C(=O)OC(C)(C)C)CCN2Cc2ccccc2)nc(NS(=O)(=O)c2cccc(C(=O)O)c2)n1 ; Cc1cccc(C)c1-c1cc2nc(n1)NS(=O)(=O)c1cccc(c1)C(=O)N1CCNC[C@@H]1CO2]

Stage 1: 3-[[4-[[(2R)-1-Benzyl-4-tert-butoxycarbonyl-piperazin-2-yl]methoxy]-6-(2,6-dimethylphenyl)pyrimidin-2-yl]sulfamoyl]benzoic acid (4.34 g, 6.310 mmol) was dissolved in methanol (40 mL) and dihydroxypalladium (800 mg, 0.5697 mmol) was added. The reaction vessel was purged with nitrogen, then hydrogen gas was bubbled through from a balloon for 15 minutes, after which the reaction was stirred at room temperature for 4 hours with the hydrogen balloon in place. The reaction mixture was then purged with nitrogen, filtered and concentrated to give a white solid 3-[[4-[[(2R)-4-tert-butoxycarbonylpiperazin-2-yl]methoxy]-6-(2,6-dimethylphenyl)pyrimidin-2-yl]sulfamoyl]benzoic acid (3.56 g, 94%) ESI-MS m/z calc. 597.2257, found 598.4 (M+1)⁺; Retention time: 0.46 minutes, LC method D.

Stage 2: The product from Stage 1 was combined with HATU (3.12 g, 8.206 mmol) in dichloromethane (500 mL), then DIPEA (5.6 mL, 32.15 mmol) was added and the reaction mixture was stirred at room temperature for 1 hour. The reaction mixture was then concentrated and dissolved in 150 mL ethyl acetate. The organic layer was washed with 100 mL 1 M HCl, and the resulting aqueous layer was extracted an additional time with 100 mL ethyl acetate. The combined organics were then washed with brine and dried over sodium sulfate. The resulting crude was purified by column chromatography on silica gel eluting with a solvent gradient of 0-10% methanol in dichloromethane, to give (moderate purity, but used in next step) tert-butyl (8R)-13-(2,6-dimethylphenyl)-2,17,17-trioxo-10-oxa-17λ⁶-thia-3,6,14,16,23-pentaazatetracyclo[16.3.1.111,15.03,8]tricosa-1(22),11,13,15(23),18,20-hexaene-6-carboxylate (2.64 g, 72%) ESI-MS m/z calc. 579.21515, found 580.3 (M+1)⁺; Retention time: 0.61 minutes, LC method D.

Stage 3: The product from stage 2 was dissolved in dichloromethane (20 mL), and HCl (35 mL of 4 M, 140.0 mmol) in dioxane and stirred for 1 hour at room temperature. The reaction mixture was then concentrated to a solid residue under vacuum. Hexanes were added and the reaction mixture was again concentrated under vacuum to give a white solid, which was used in the next step without further purification: (8R)-13-(2,6-dimethylphenyl)-10-oxa-17λ⁶-thia-3,6,14,16,23-pentaazatetracyclo[16.3.1.111,15.03,8]tricosa-1(21),11,13,15(23),18(22),19-hexaene-2,17,17-trione (hydrochloride salt) (2.32 g, 71%) ESI-MS m/z calc. 479.16272, found 480.4 (M+1)⁺; Retention time: 0.3 minutes, LC method D.

A 10 mg portion of the material from stage 3 was further purified by preparative HPLC (1-40 MeCN in water, HCl Modifier 15 min run), to give (8R)-13-(2,6-dimethylphenyl)-10-oxa-17λ⁶-thia-3,6,14,16,23-pentaazatetracyclo[16.3.1.111,15.03,8]tricosa-1(21),11,13,15(23),18(22),19-hexaene-2,17,17-trione (hydrochloride salt) (6 mg, 0%). ESI-MS m/z calc. 479.16272, found 480.4 (M+1)⁺; Retention time: 0.7 minutes (LC method A).

### Example 72: Preparation of Compound 162

### Step 1: (8R)-13-(2,6-Dimethylphenyl)-6-methyl-10-oxa-17λ⁶-thia-3,6,14,16,23-pentaazatetracyclo[16.3.1.111,15.03,8]tricosa-1(21),11,13,15(23),18(22),19-hexaene-2,17,17-trione (Compound 162)

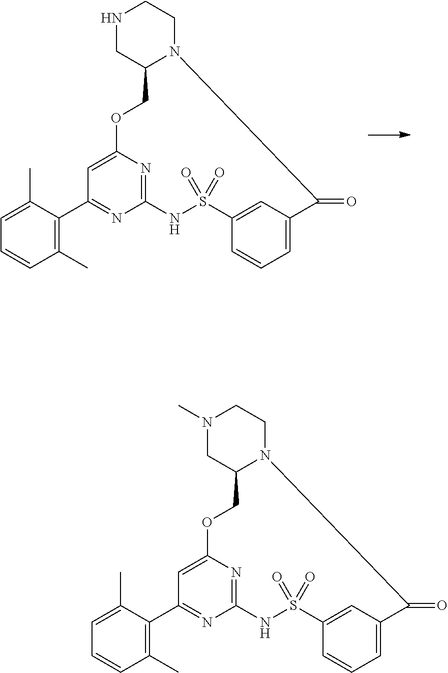 [SMILES: Cc1cccc(C)c1-c1cc2nc(n1)NS(=O)(=O)c1cccc(c1)C(=O)N1CCNC[C@@H]1CO2 ; Cc1cccc(C)c1-c1cc2nc(n1)NS(=O)(=O)c1cccc(c1)C(=O)N1CCN(C)C[C@@H]1CO2]

In a 3-mL vial, (8R)-13-(2,6-dimethylphenyl)-10-oxa-17λ⁶-thia-3,6,14,16,23-pentaazatetracyclo[16.3.1.111,15.03,8]tricosa-1(21),11,13,15(23),18(22),19-hexaene-2,17,17-trione (hydrochloride salt) (14 mg, 0.02713 mmol) was dissolved in formic acid (400 μL) and 37% aqueous formaldehyde (400 μL, 14.52 mmol). This solution was stirred at 70° C. for 4 h. The reaction mixture was then cooled to room temperature, diluted with methanol, filtered, and purified by reverse phase HPLC (1-40% ACN in water, with HCl modifier, 15 min run), and concentrated to give as a white powder, (8R)-13-(2,6-dimethylphenyl)-6-methyl-10-oxa-17λ⁶-thia-3,6,14,16,23-pentaazatetracyclo[16.3.1.111,15.03,8]tricosa-1(21),11,13,15(23),18(22),19-hexaene-2,17,17-trione (hydrochloride salt) (5.1 mg, 35%) ESI-MS m/z calc. 493.17838, found 494.4 (M+1)⁺; Retention time: 0.8 minutes, LC method A.

### Example 73: Preparation of Compound 163

### Step 1: (8R)-13-(2,6-Dimethylphenyl)-6-{2-[1-(trifluoromethyl)cyclopropyl]ethyl}-10-oxa-17λ⁶-thia-3,6,14,16,23-pentaazatetracyclo[16.3.1.111,15.03,8]tricosa-1(22),11(23),12,14,18,20-hexaene-2,17,17-trione (Compound 163)

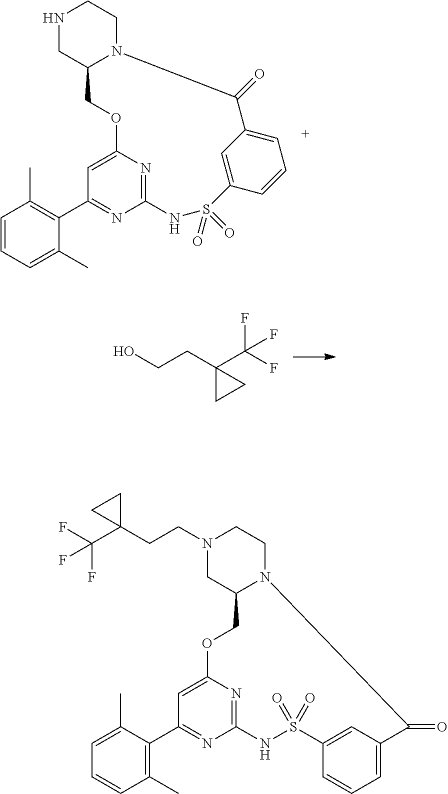 [SMILES: Cc1cccc(C)c1-c1cc2nc(n1)NS(=O)(=O)c1cccc(c1)C(=O)N1CCNC[C@@H]1CO2 ; OCCC1(C(F)(F)F)CC1 ; Cc1cccc(C)c1-c1cc2nc(n1)NS(=O)(=O)c1cccc(c1)C(=O)N1CCN(CCC3(C(F)(F)F)CC3)C[C@@H]1CO2]

2-[1-(Trifluoromethyl)cyclopropyl]ethanol (225 mg, 1.460 mmol) and Dess-martin periodinane (615 mg, 1.450 mmol) were combined in dichloromethane (3 mL) and stirred at room temperature for 1 hour, then the contents of the vial were added to a separate vial containing (8R)-13-(2,6-dimethylphenyl)-10-oxa-17λ⁶-thia-3,6,14,16,23-pentaazatetracyclo[16.3.1.111,15.03,8]tricosa-1(21),11,13,15(23),18(22),19-hexaene-2,17,17-trione (hydrochloride salt) (125 mg, 0.2422 mmol) dissolved in acetic acid (3 mL). After stirring for 30 minutes at room temperature, sodium triacetoxyborohydride (412 mg, 1.944 mmol) was added. The reaction mixture was stirred for an additional 20 minutes, then concentrated, dissolved in DMSO and methanol, filtered, and purified by reverse phase HPLC (1-70% ACN in water, 15 min run) to give (8R)-13-(2,6-dimethylphenyl)-6-{2-[1-(trifluoromethyl)cyclopropyl]ethyl}-10-oxa-17λ⁶-thia-3,6,14,16,23-pentaazatetracyclo[16.3.1.111,15.03,8]tricosa-1(22),11(23),12,14,18,20-hexaene-2,17,17-trione (hydrochloride salt) (60 mg, 37%). ¹H NMR (400 MHz, DMSO) δ 11.21 (s, 1H), 8.53 (s, 1H), 7.96 (d, J=7.1 Hz, 1H), 7.71 (d, J=7.8 Hz, 2H), 7.27 (t, J=7.5 Hz, 1H), 7.13 (d, J=7.7 Hz, 2H), 6.30 (s, 1H), 5.27 (s, 1H), 5.14 (s, 1H), 4.63 (d, J=14.7 Hz, 1H), 4.13 (s, 1H), 3.57 (d, J=21.9 Hz, 2H), 3.25 (m, 3H), 3.04 (s, 2H), 2.19-1.96 (m, 8H), 0.98 (d, J=6.9 Hz, 2H), 0.86 (d, J 5.6 Hz, 2H). ESI-MS m/z calc. 615.2127, found 616.3 (M+1)⁺; Retention time: 1.24 minutes, LC method A.

### Example 74: Preparation of Compound 164

### Step 1: (8R)-6-Benzyl-13-(2,6-dimethylphenyl)-10-oxa-17λ⁶-thia-3,6,14,16,23-pentaazatetracyclo[16.3.1.111,15.03,8]tricosa-1(21),11,13,15(23),18(22),19-hexaene-2,17,17-trione (Compound 164)

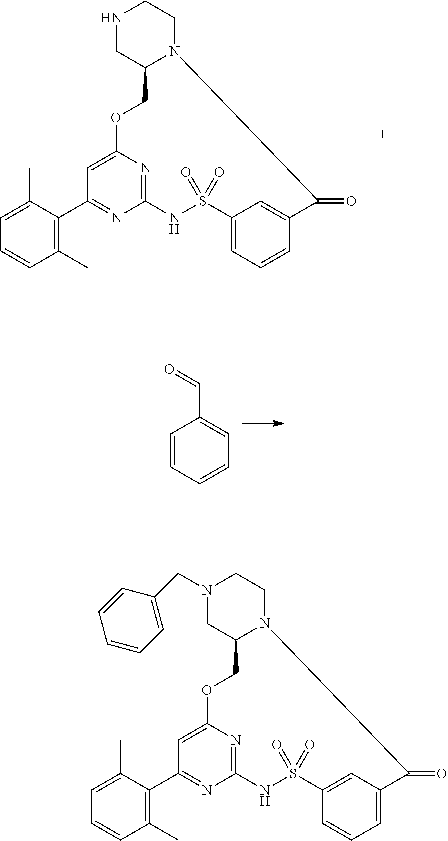 [SMILES: Cc1cccc(C)c1-c1cc2nc(n1)NS(=O)(=O)c1cccc(c1)C(=O)N1CCNC[C@@H]1CO2 ; O=Cc1ccccc1 ; Cc1cccc(C)c1-c1cc2nc(n1)NS(=O)(=O)c1cccc(c1)C(=O)N1CCN(Cc3ccccc3)C[C@@H]1CO2]

(8R)-13-(2,6-Dimethylphenyl)-10-oxa-17λ⁶-thia-3,6,14,16,23-pentaazatetracyclo[16.3.1.111,15.03,8]tricosa-1(21),11,13,15(23),18(22),19-hexaene-2,17,17-trione (12.000 mg, 0.02502 mmol), and the corresponding benzaldehyde (approximately 13.28 mg, 12.72 μL, 0.1251 mmol) were combined in acetic acid. After 5 minutes, Sodium triacetoxyborohydride (approximately 31.81 mg, 0.1501 mmol) was added and the reaction was stirred for an additional 30 minutes at room temperature. The reaction mixture was then diluted with methanol, filtered and purified by reverse phase HPLC (1-70% or 1-40% ACN in water, with HCl modifier, 15 min run) to give the corresponding (8R)-6-benzyl-13-(2,6-dimethylphenyl)-10-oxa-17λ⁶-thia-3,6,14,16,23-pentaazatetracyclo[16.3.1.111,15.03,8]tricosa-1(21),11,13,15(23),18(22),19-hexaene-2,17,17-trione (hydrochloride salt) (8 mg, 52%). ESI-MS m/z calc. 569.20966, found 570.4 (M+1)⁺; Retention time: 1.16 minutes; LC method A.

### Example 75: Preparation of Compound 165

### Step 1: (8R)-13-(2,6-Dimethylphenyl)-6-(propan-2-yl)-10-oxa-17λ⁶-thia-3,6,14,16,23-pentaazatetracyclo[16.3.1.111,15.03,8]tricosa-1(21),11,13,15(23),18(22),19-hexaene-2,17,17-trione (Compound 165)

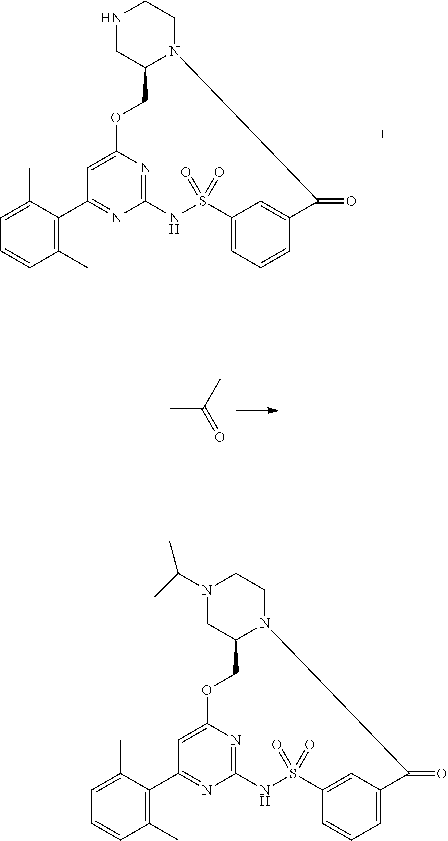

(8R)-13-(2,6-dimethylphenyl)-10-oxa-17λ⁶-thia-3,6,14,16,23-pentaazatetracyclo[16.3.1.111,15.03,8]tricosa-1(21),11,13,15(23),18(22),19-hexaene-2,17,17-trione (12.000 mg, 0.02502 mmol), and the corresponding acetone (approximately 7.266 mg, 9.186 μL, 0.1251 mmol) were combined in acetic acid. After 5 minutes, Sodium triacetoxyborohydride (approximately 31.81 mg, 0.1501 mmol) was added and the reaction was stirred for an additional 30 minutes at room temperature. The reaction mixture was then diluted with methanol, filtered and purified by reverse phase HPLC (1-70% or 1-40% ACN in water, with HCl modifier, 15 min run) to give the corresponding (8R)-13-(2,6-dimethylphenyl)-6-(propan-2-yl)-10-oxa-17λ⁶-thia-3,6,14,16,23-pentaazatetracyclo[16.3.1.111,15.03,8]tricosa-1(21),11,13,15(23),18(22),19-hexaene-2,17,17-trione (hydrochloride salt) (4.5 mg, 32%). ESI-MS m/z calc. 521.20966, found 522.4 (M+1)⁺; Retention time: 0.86 minutes; LC method A.

### Example 76: Preparation of Compound 166

### Step 1: (8R)—N-Cycloheptyl-13-(2,6-dimethylphenyl)-2,17,17-trioxo-10-oxa-17λ⁶-thia-3,6,14,16,23-pentaazatetracyclo[16.3.1.111,15.03,8]tricosa-1(21),11,13,15(23),18(22),19-hexaene-6-carboxamide (Compound 166)

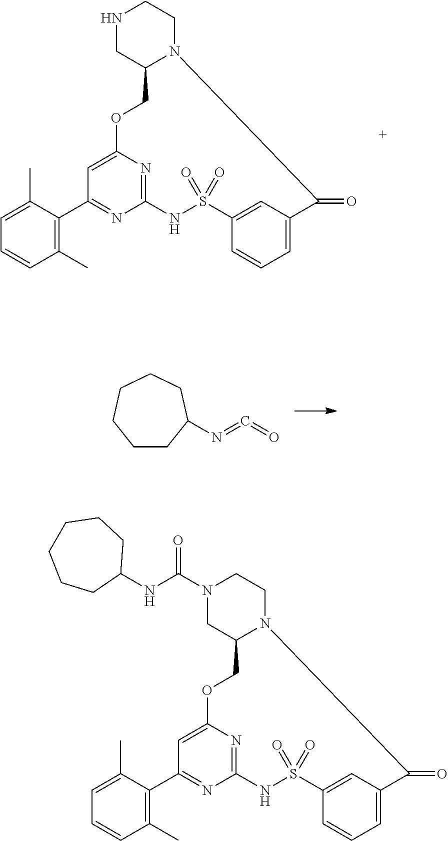 [SMILES: Cc1cccc(C)c1-c1cc2nc(n1)NS(=O)(=O)c1cccc(c1)C(=O)N1CCNC[C@@H]1CO2 ; O=C=NC1CCCCCC1 ; Cc1cccc(C)c1-c1cc2nc(n1)NS(=O)(=O)c1cccc(c1)C(=O)N1CCN(C(=O)NC3CCCCCC3)C[C@@H]1CO2]

(8R)-13-(2,6-dimethylphenyl)-10-oxa-17λ⁶-thia-3,6,14,16,23-pentaazatetracyclo[16.3.1.111,15.03,8]tricosa-1(21),11,13,15(23),18(22),19-hexaene-2,17,17-trione (hydrochloride salt) (12 mg, 0.02326 mmol) and isocyanatocycloheptane (approximately 4.857 mg, 4.621 μL, 0.03489 mmol) were dissolved in THE (0.5 mL), and diisopropylethylamine (approximately 12.02 mg, 16.20 μL, 0.09304 mmol) was added. After stirring at room temperature for 1 hour, the reaction mixture was diluted with methanol, filtered, and purified by reverse phase HPLC (1-70% ACN in water, HCl modifier, 15 minutes) to give (8R)—N-cycloheptyl-13-(2,6-dimethylphenyl)-2,17,17-trioxo-10-oxa-17λ⁶-thia-3,6,14,16,23-pentaazatetracyclo[16.3.1.111,15.03,8]tricosa-1(21),11,13,15(23),18(22),19-hexaene-6-carboxamide (8.1 mg, 56%). ESI-MS m/z calc. 618.26245, found 619.4 (M+1)⁺; Retention time: 1.6 minutes; LC method A.

### Example 77: Preparation of Compound 167

### Step 1: (8R)-13-(2,6-Dimethylphenyl)-2,17,17-trioxo-N,N-bis(propan-2-yl)-10-oxa-17λ⁶-thia-3,6,14,16,23-pentaazatetracyclo[16.3.1.111,15.03,8]tricosa-1(21),11,13,15(23),18(22),19-hexaene-6-carboxamide (Compound 167)

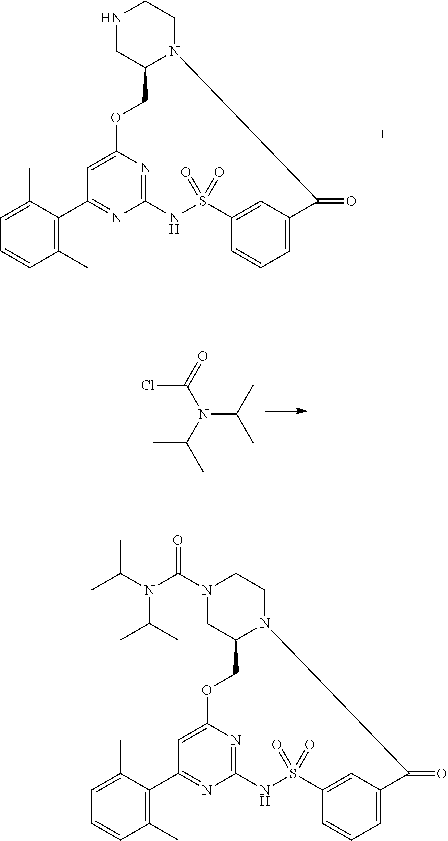

(8R)-13-(2,6-dimethylphenyl)-10-oxa-17λ⁶-thia-3,6,14,16,23-pentaazatetracyclo[16.3.1.111,15.03,8]tricosa-1(21),11,13,15(23),18(22),19-hexaene-2,17,17-trione (hydrochloride salt) (approximately 12.00 mg, 0.02326 mmol), and N,N-diisopropylcarbamoyl chloride (approximately 15.23 mg, 0.09304 mmol) were combined in dichloromethane (0.3 mL), and triethylamine (approximately 18.83 mg, 0.1861 mmol) was added. The reaction mixture was stirred at room temperature for 2 hours. The reaction mixture were then diluted with methanol, filtered, and purified by reverse phase HPLC (1-70% ACN in water, 15 min run, HCl modifier) to give upon concentration (8R)-13-(2,6-dimethylphenyl)-2,17,17-trioxo-N,N-bis(propan-2-yl)-10-oxa-17λ⁶-thia-3,6,14,16,23-pentaazatetracyclo[16.3.1.111,15.03,8]tricosa-1(21),11,13,15(23),18(22),19-hexaene-6-carboxamide (6.5 mg, 46%). ESI-MS m/z calc. 606.26245, found 607.4 (M+1)⁺; Retention time: 1.67 minutes; LC method A.

### Example 78: Preparation of Compound 168

### Step 1: (8R)-6-Cyclohexanecarbonyl-13-(2,6-dimethylphenyl)-10-oxa-17λ⁶-thia-3,6,14,16,23-pentaazatetracyclo[16.3.1.111,15.03,8]tricosa-1(21),11,13,15(23),18(22),19-hexaene-2,17,17-trione (Compound 168)

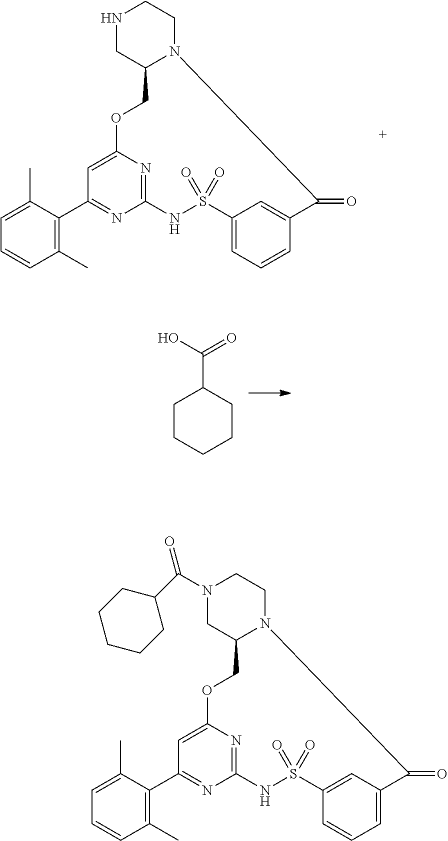 [SMILES: Cc1cccc(C)c1-c1cc2nc(n1)NS(=O)(=O)c1cccc(c1)C(=O)N1CCNC[C@@H]1CO2 ; O=C(O)C1CCCCC1 ; Cc1cccc(C)c1-c1cc2nc(n1)NS(=O)(=O)c1cccc(c1)C(=O)N1CCN(C(=O)C3CCCCC3)C[C@@H]1CO2]

(8R)-13-(2,6-Dimethylphenyl)-10-oxa-17λ⁶-thia-3,6,14,16,23-pentaazatetracyclo[16.3.1.111,15.03,8]tricosa-1(21),11,13,15(23),18(22),19-hexaene-2,17,17-trione (hydrochloride salt) (12 mg, 0.02326 mmol), the cyclohexanecarboxylic acid (approximately 3.876 mg, 0.03024 mmol), and HATU (approximately 11.50 mg, 0.03024 mmol) were combined in DMF (0.5 mL), and DIPEA (approximately 15.03 mg, 20.26 μL, 0.1163 mmol) was added. The reaction mixture was stirred at room temperature for one hour, then filtered and purified by reverse phase HPLC (1-70% ACN in water, with HCl) and dried to give the (8R)-6-cyclohexanecarbonyl-13-(2,6-dimethylphenyl)-10-oxa-17λ⁶-thia-3,6,14,16,23-pentaazatetracyclo[16.3.1.111,15.03,8]tricosa-1(21),11,13,15(23),18(22),19-hexaene-2,17,17-trione (9.7 mg, 71%). ESI-MS m/z calc. 589.2359, found 590.4 (M+1)⁺; Retention time: 1.59 minutes; LC method A.

### Example 79: Preparation of Compound 169

### Step 1: tert-Butyl 4-benzyl-3-(hydroxymethyl)piperazine-1-carboxylate

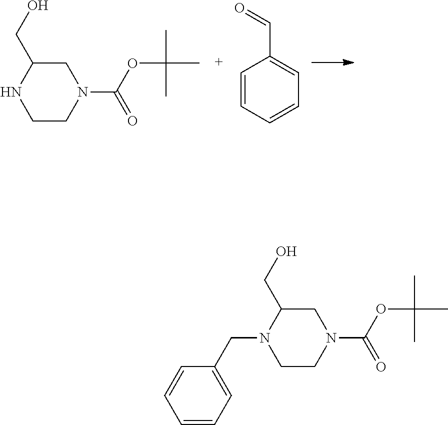

tert-Butyl 3-(hydroxymethyl)piperazine-1-carboxylate (7 g, 32.37 mmol) and benzaldehyde (3.9 mL, 38.37 mmol) were combined in DCE (100 mL) with acetic acid (2.5 mL, 43.96 mmol), and stirred for 30 minutes at room temperature. The reaction mixture was then cooled to 0° C., and sodium triacetoxyborohydride (9 g, 42.46 mmol) was added. The reaction mixture was returned to room temperature and stirred for 16 hours. The reaction mixture was poured into aqueous sodium bicarbonate and extracted 3× ethyl acetate. The combined organic layers were washed with brine, dried over sodium sulfate, and concentrated. The resulting crude was purified by chromatography on silica gel, eluting with a gradient of 0-100% ethyl acetate in dichloromethane. tert-butyl 4-benzyl-3-(hydroxymethyl)piperazine-1-carboxylate (5.1 g, 51%) ESI-MS m/z calc. 306.19434, found 307.3 (M+1)⁺; Retention time: 0.35 minutes, LC method D.

### Step 2: 3-[[4-[(1-Benzyl-4-tert-butoxycarbonyl-piperazin-2-yl)methoxy]-6-(2,6-dimethylphenyl)pyrimidin-2-yl]sulfamoyl]benzoic acid

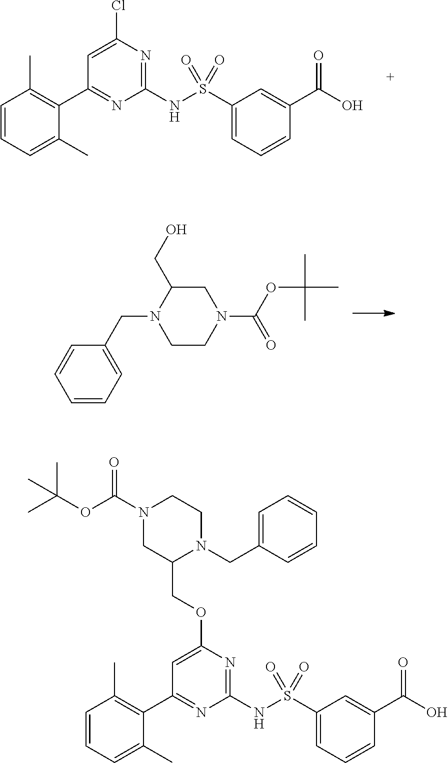

3-[[4-Chloro-6-(2,6-dimethylphenyl)pyrimidin-2-yl]sulfamoyl]benzoic acid (900 mg, 2.154 mmol) and tert-butyl 4-benzyl-3-(hydroxymethyl)piperazine-1-carboxylate (870 mg, 2.839 mmol) were combined in anhydrous NMP (12 mL). The reaction mixture was cooled to 0° C., and NaH (430 mg, 10.75 mmol) was slowly added in portions under a stream of nitrogen. The reaction mixture was then stirred at room temperature for 1 hour. After this time, the reaction mixture was slowly added to a flask containing aqueous ammonium chloride and ethyl acetate. The aqueous and organic layers were separated, and the aqueous was extracted an additional 4× ethyl acetate. The combined organics were washed with water, brine and dried over sodium sulfate. The resulting crude material was purified by chromatography on silica gel, eluting with 0-10% methanol in dichloromethane. Fractions containing product were combined and concentrated to give 3-[[4-[(1-benzyl-4-tert-butoxycarbonyl-piperazin-2-yl)methoxy]-6-(2,6-dimethylphenyl)pyrimidin-2-yl]sulfamoyl]benzoic acid (1.103 g, 74%) ESI-MS m/z calc. 687.27264, found 688.5 (M+1)⁺; Retention time: 0.54 minutes, LC method D.

### Step 3: 13-(2,6-Dimethylphenyl)-10-oxa-17λ⁶-thia-3,6,14,16,23-pentaazatetracyclo[16.3.1.111,15.03,8]tricosa-1(21),11,13,15(23),18(22),19-hexaene-2,17,17-trione

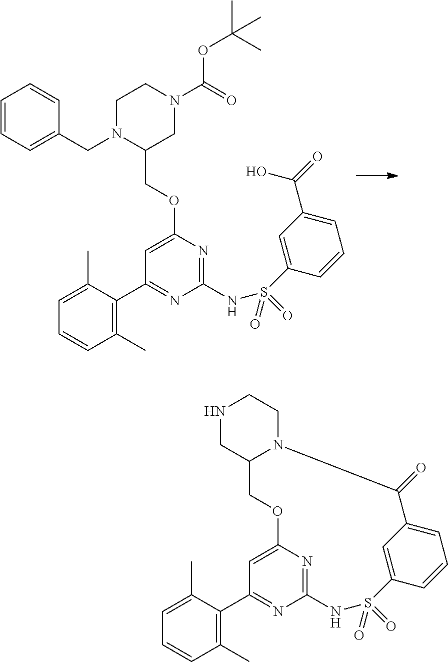 [SMILES: Cc1cccc(C)c1-c1cc(OCC2CN(C(=O)OC(C)(C)C)CCN2Cc2ccccc2)nc(NS(=O)(=O)c2cccc(C(=O)O)c2)n1 ; Cc1cccc(C)c1-c1cc2nc(n1)NS(=O)(=O)c1cccc(c1)C(=O)N1CCNCC1CO2]

Stage 1: 3-[[4-[(1-Benzyl-4-tert-butoxycarbonyl-piperazin-2-yl)methoxy]-6-(2,6-dimethylphenyl)pyrimidin-2-yl]sulfamoyl]benzoic acid (500 mg, 0.7270 mmol) was dissolved in methanol (10 mL) and dihydroxypalladium (225 mg, 0.3204 mmol) was added. The reaction vessel was purged with nitrogen, then hydrogen gas was bubbled through from a balloon for 15 minutes, after which the reaction was stirred at room temperature for 2 hours with the hydrogen balloon in place. The reaction mixture was then purged with nitrogen, filtered and concentrated to give a white solid 3-[[4-[(4-tert-butoxycarbonylpiperazin-2-yl)methoxy]-6-(2,6-dimethylphenyl)pyrimidin-2-yl]sulfamoyl]benzoic acid (411 mg, 95%) ESI-MS m/z calc. 597.2257, found 598.3 (M+1)⁺; Retention time: 0.46 minutes (LC method D).

Stage 2: The product from stage 1 was combined with HATU (360 mg, 0.9468 mmol) in DMF (50 mL), then DIPEA (650 μL, 3.732 mmol) was added and the reaction mixture was stirred at room temperature for 2 hours. The reaction mixture was then diluted with 0.3 M HCl and extracted 3× with ethyl acetate. The combined organics were then washed with water 2×, then brine, and dried over sodium sulfate. The resulting crude was purified by column chromatography on silica gel eluting with a solvent gradient of 0-10% methanol in dichloromethane, to give tert-butyl 13-(2,6-dimethylphenyl)-2,17,17-trioxo-10-oxa-17λ⁶-thia-3,6,14,16,23-pentaazatetracyclo[16.3.1.111,15.03,8]tricosa-1(21),11,13,15(23),18(22),19-hexaene-6-carboxylate (340 mg, 81%) ESI-MS m/z calc. 579.21515, found 580.3 (M+1)⁺; Retention time: 0.61 minutes, (LC method D).

Stage 3: The product from Stage 2 was dissolved in dichloromethane (4 mL), and HCl (4 mL of 4 M, 16.00 mmol) and stirred for 1 hour at room temperature. The reaction mixture was then concentrated to a solid residue under vacuum. Hexanes were added and the reaction mixture was again concentrated under vacuum to give a white solid, which was used in the next step without further purification: 13-(2,6-dimethylphenyl)-10-oxa-17λ⁶-thia-3,6,14,16,23-pentaazatetracyclo[16.3.1.111,15.03,8]tricosa-1(21),11,13,15(23),18(22),19-hexaene-2,17,17-trione (281 mg, 81%). ESI-MS m/z calc. 479.16272, found 480.3 (M+1)⁺; Retention time: 0.27 minutes (LC method D).

### Step 4: 6-Benzyl-13-(2,6-dimethylphenyl)-10-oxa-17λ⁶-thia-3,6,14,16,23-pentaazatetracyclo[16.3.1.111,15.03,8]tricosa-1(21),11,13,15(23),18(22),19-hexaene-2,17,17-trione (Compound 169)

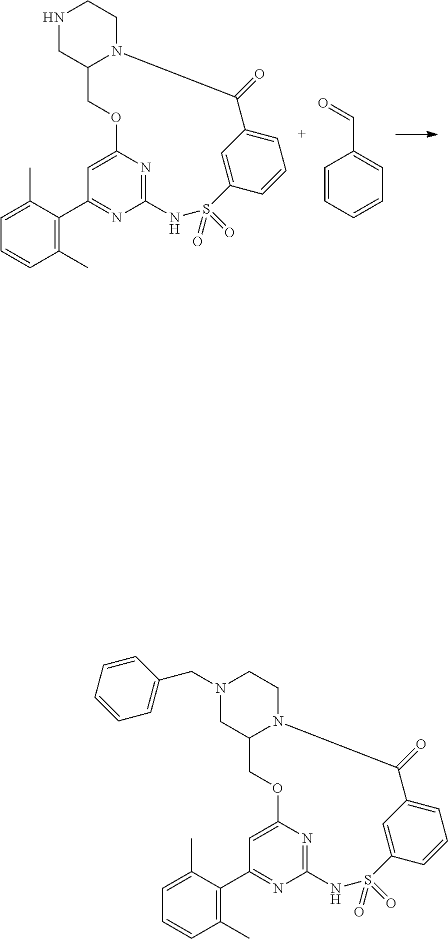

13-(2,6-Dimethylphenyl)-10-oxa-17λ⁶-thia-3,6,14,16,23-pentaazatetracyclo[16.3.1.111,15.03,8]tricosa-1(21),11,13,15(23),18(22),19-hexaene-2,17,17-trione (12 mg, 0.02502 mmol) was combined in acetic acid (0.5 mL) with benzaldehyde (13 mg, 0.1225 mmol). The reaction was stirred at room temperature for 5 minutes, then sodium triacetoxyborohydride (approximately 31.81 mg, 0.1501 mmol) was added and the reaction mixture was stirred at room temperature for 20 minutes. The reaction mixture was then diluted with 0.2 mL methanol, filtered, and purified by reverse phase HPLC (1-40% ACN or 1-70% ACN 15 min run with HCl modifier) to give 6-benzyl-13-(2,6-dimethylphenyl)-10-oxa-17λ⁶-thia-3,6,14,16,23-pentaazatetracyclo[16.3.1.111,15.03,8]tricosa-1(21),11,13,15(23),18(22),19-hexaene-2,17,17-trione (hydrochloride salt) (7 mg, 46%). ESI-MS m/z calc. 569.20966, found 570.3 (M+1)⁺; Retention time: 1.1 minutes; LC method A.

### Example 80: Preparation of Compound 170

### Step 1: tert-Butyl 2-(hydroxymethyl)-4-[2-[1-(trifluoromethyl)cyclopropyl]ethyl]piperazine-1-carboxylate

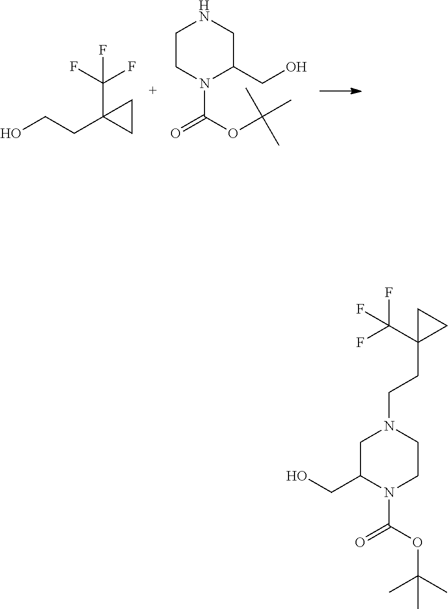

2-[1-(Trifluoromethyl)cyclopropyl]ethanol (approximately 427.6 mg, 2.774 mmol) was dissolved in 4 mL DCE and Dess-Martin Periodinane (approximately 1.236 g, 2.913 mmol) was added and the reaction was stirred at room temperature for one hour. This reaction mixture was then added to a vial containing tert-butyl 2-(hydroxymethyl)piperazine-1-carboxylate (300 mg, 1.387 mmol) in 2 mL DCE. acetic acid (500 μL, 8.792 mmol) was then added and the reaction mixture was stirred at room temperature for an additional two hours. At this point, sodium triacetoxyborohydride (1.3 g, 6.134 mmol) was added and the reaction mixture was allowed to stir an additional 16 hours at room temperature. The reaction mixture was then poured into aqueous sodium bicarbonate and extracted 3× ethyl acetate. The combined organics were washed with brine, dried over sodium sulfate, and concentrated. The resulting crude was purified by chromatography on silica gel, eluting with 0-100% ethyl acetate in dichloromethane (comes out fairly late in the run, not UV active but detected by ELSD), to give tert-butyl 2-(hydroxymethyl)-4-[2-[1-(trifluoromethyl)cyclopropyl]ethyl]piperazine-1-carboxylate (269 mg, 55%) as a colorless oil. ESI-MS m/z calc. 352.1974, found 353.4 (M+1)⁺; Retention time: 0.48 minutes; LC method D.

### Step 2: 13-(2,6-Dimethylphenyl)-6-{2-[1-(trifluoromethyl)cyclopropyl]ethyl}-10-oxa-17λ⁶-thia-3,6,14,16,23-pentaazatetracyclo[16.3.1.111,15.03,8]tricosa-1(22),11,13,15(23),18,20-hexaene-2,17,17-trione (Compound 170)

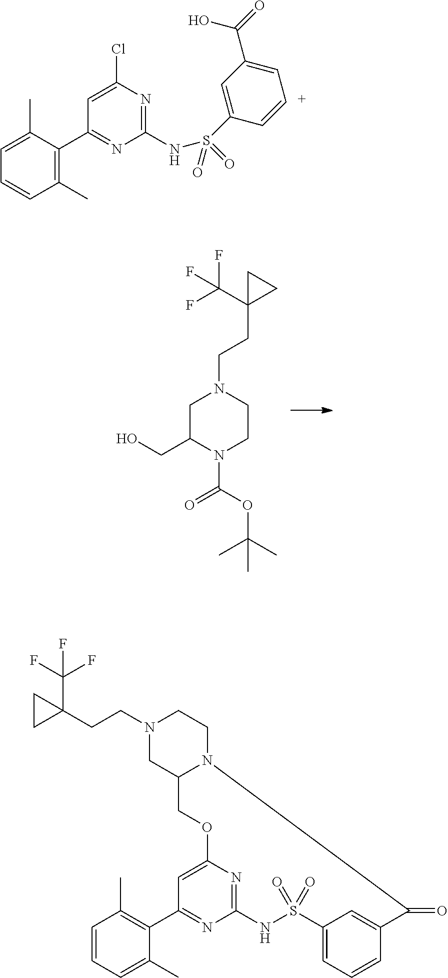 [SMILES: Cc1cccc(C)c1-c1cc(Cl)nc(NS(=O)(=O)C2=CC(C(=O)O)=C[C+]=C2)n1 ; CC(C)(C)OC(=O)N1CCN(CCC2(C(F)(F)F)CC2)CC1CO ; Cc1cccc(C)c1-c1cc2nc(n1)NS(=O)(=O)c1cccc(c1)C(=O)N1CCN(CCC3(C(F)(F)F)CC3)CC1CO2]

Stage 1: 3-[[4-Chloro-6-(2,6-dimethylphenyl)pyrimidin-2-yl]sulfamoyl]benzoic acid (30 mg, 0.07179 mmol) and tert-butyl 2-(hydroxymethyl)-4-[2-[1-(trifluoromethyl)cyclopropyl]ethyl]piperazine-1-carboxylate (50 mg, 0.1419 mmol) were combined with sodium tert-butoxide (30 mg, 0.3122 mmol) in anhydrous THE (0.5 mL) and stirred at room temperature. After 2 hours, additional sodium tert-butoxide (17 mg, 0.1769 mmol) was added and the reaction was allowed to stir for an additional 16 hours. The reaction mixture was quenched with acetic acid, diluted with methanol, filtered and purified by reverse phase HPLC (1-70% ACN, HCl modifier, 15 min run) to give 3-[[4-[[1-tert-butoxycarbonyl-4-[2-[1-(trifluoromethyl)cyclopropyl]ethyl]piperazin-2-yl]methoxy]-6-(2,6-dimethylphenyl)pyrimidin-2-yl]sulfamoyl]benzoic acid (hydrochloride salt) (8 mg, 14%) ESI-MS m/z calc. 733.2757, found 734.4 (M+1)⁺; Retention time: 0.56 minutes (LC method D).

Stage 2: The boc-protected product was dissolved in dichloromethane (0.5 mL) with HCl (500 μL of 4 M, 2.000 mmol) and stirred for 90 minutes at room temperature. The boc-deprotected material from the reverse phase HPLC from step 1 was then added dissolved in 1 mL dichloromethane, and the reaction mixture was concentrated to give 3-[[4-(2,6-dimethylphenyl)-6-[[4-[2-[1-(trifluoromethyl)cyclopropyl]ethyl]piperazin-2-yl]methoxy]pyrimidin-2-yl]sulfamoyl]benzoic acid (hydrochloride salt) (10 mg, 21%) ESI-MS m/z calc. 633.22327, found 634.4 (M+1)⁺; Retention time: 0.46 minutes (LC method D).

Stage 3: The product from stage 2 was combined with HATU (8 mg, 0.02104 mmol) in DMF (0.8 mL), and DIPEA (17 μL, 0.09760 mmol) was added. After stirring for 1 hour at room temperature, the reaction mixture was diluted with 0.3 mL methanol, filtered, and purified by reverse phase HPLC (1-70% ACN, HCl modifier, 15 min run), to give 13-(2,6-dimethylphenyl)-6-{2-[1-(trifluoromethyl)cyclopropyl]ethyl}-10-oxa-17λ⁶-thia-3,6,14,16,23-pentaazatetracyclo[16.3.1.111,15.03,8]tricosa-1(22),11,13,15(23),18,20-hexaene-2,17,17-trione (hydrochloride salt) (6.5 mg, 14%) ESI-MS m/z calc. 615.2127, found 616.3 (M+1)⁺; Retention time: 1.19 minutes (LC method A).

### Example 81: Preparation of Compound 171

### Step 1: tert-Butyl (3S)-4-[3-[[4-chloro-6-(2,6-dimethylphenyl)pyrimidin-2-yl]sulfamoyl]benzoyl]-3-(hydroxymethyl)piperazine-1-carboxylate (Compound 171)

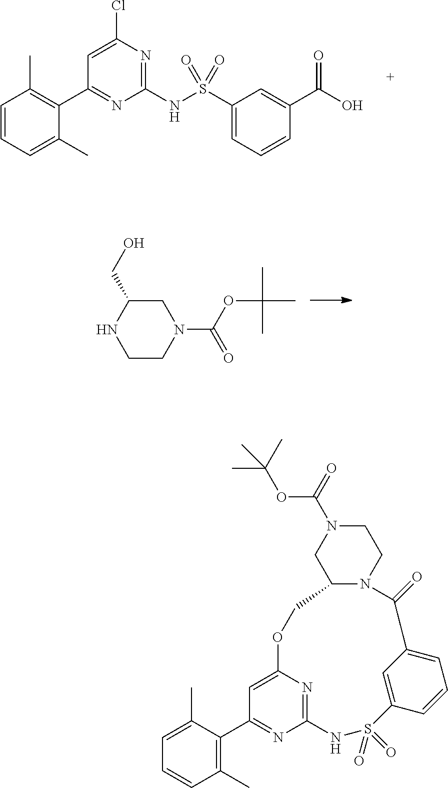 [SMILES: Cc1cccc(C)c1-c1cc(Cl)nc(NS(=O)(=O)c2cccc(C(=O)O)c2)n1 ; CC(C)(C)OC(=O)N1CCN[C@H](CO)C1 ; Cc1cccc(C)c1-c1cc2nc(n1)NS(=O)(=O)c1cccc(c1)C(=O)N1CCN(C(=O)OC(C)(C)C)C[C@H]1CO2]

3-[[4-Chloro-6-(2,6-dimethylphenyl)pyrimidin-2-yl]sulfamoyl]benzoic acid (100 mg, 0.2393 mmol), tert-butyl (3S)-3-(hydroxymethyl)piperazine-1-carboxylate (55 mg, 0.2543 mmol), and EDC (hydrochloride salt) (66 mg, 0.2893 mmol) were combined in anhydrous dichloromethane (1.2 mL) at room temperature and stirred for 20 minutes. The reaction mixture was then added to 20 mL ethyl acetate and 20 mL 0.5 M HCl. The layers were separated and the aqueous was extracted a second time with 20 mL ethyl acetate. The combined organics were washed with brine, dried over sodium sulfate and concentrated. The crude material was purified by chromatography on silica gel eluting with 0-100% ethyl acetate in hexanes. The fractions containing product were concentrated to give the intermediate, tert-butyl (3S)-4-[3-[[4-chloro-6-(2,6-dimethylphenyl)pyrimidin-2-yl]sulfamoyl]benzoyl]-3-(hydroxymethyl)piperazine-1-carboxylate (54 mg, 37%). ESI-MS m/z calc. 615.19183, found 616.2 (M+1)⁺; Retention time: 0.71 minutes; LC method D. The amide product was combined with NaH (30 mg, 0.7501 mmol) in anhydrous NMP (20 mL) in a nitrogen-purged vial and heated to 70° C. for 2 hours. The reaction mixture was then quenched with 1M HCl, and poured into 50 mL 0.5 M HCl, and 50 mL ethyl acetate. The layers were separated and the aqueous was extracted 2×30 mL ethyl acetate. The combined organics were washed 5 times with water, brine, dried over sodium sulfate and concentrated. The resulting crude was purified by column chromatography on silica gel using a gradient of 0-10% methanol in dichloromethane. The fractions containing product were combined and concentrated to give tert-butyl (8S)-13-(2,6-dimethylphenyl)-2,17,17-trioxo-10-oxa-17λ⁶-thia-3,6,14,16,23-pentaazatetracyclo[16.3.1.111,15.03,98]tricosa-1(22),11(23),12,14,18,20-hexaene-6-carboxylate (26 mg, 19%) ESI-MS m/z calc. 579.21515, found 580.2 (M+1)⁺; Retention time: 1.62 minutes (LC method A).

### Example 82: Preparation of Compound 172 and Compound 173

### Step 1: tert-Butyl 4-benzyl-3-[methoxy(methyl)carbamoyl]piperazine-1-carboxylate

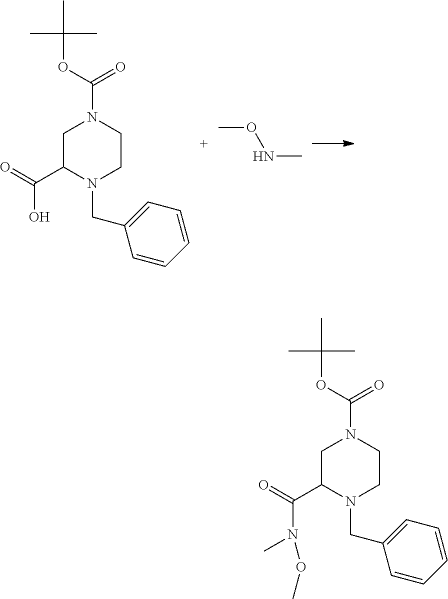

A DMF (10 mL) mixture of 1-benzyl-4-tert-butoxycarbonyl-piperazine-2-carboxylic acid (1.0234 g, 3.194 mmol), N-methoxymethanamine (hydrochloride salt) (521.5 mg, 5.346 mmol), DIPEA (1.7 mL, 9.760 mmol), EDCI (771.7 mg, 4.026 mmol), and HOBt (473.8 mg, 3.506 mmol) was stirred at room temperature for 4 hours and then quenched with a saturated aqueous solution of ammonium chloride (10 mL) and extracted with ethyl acetate (15 ml×2). The organic layer was washed with water (10 mL) followed by brine (10 mL), dried over anhydrous sodium sulfate, filtered, and concentrated in vacuo to give tert-butyl 4-benzyl-3-[methoxy(methyl)carbamoyl]piperazine-1-carboxylate (1.2667 g, 109%) ESI-MS m/z calc. 363.21582, found 364.2 (M+1)⁺; Retention time: 1.0 minutes, LC method A.

### Step 2: tert-Butyl 4-benzyl-3-(1-hydroxyethyl)piperazine-1-carboxylate

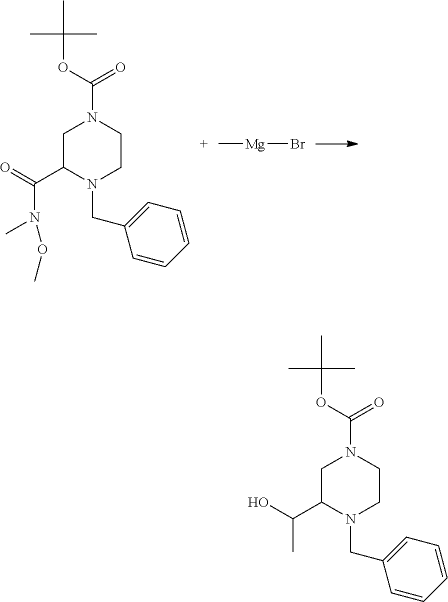

Stage 1: A THE (5 mL) mixture of tert-butyl 4-benzyl-3-[methoxy(methyl)carbamoyl]piperazine-1-carboxylate (400.6 mg, 1.102 mmol) was cooled to 0° C. and treated with bromo(methyl)magnesium (750 μL of 3 M, 2.250 mmol), added dropwise. The reaction mixture was warmed to room temperature and stirred for 16 h. The reaction mixture was treated with an aqueous solution of saturated ammonium chloride (10 mL) and extracted with ethyl acetate (2×10 mL). The organic layer was dried over anhydrous sodium sulfate, filtered, and concentrated in vacuo to give tert-butyl 3-acetyl-4-benzyl-piperazine-1-carboxylate (416.2 mg, 119%) ESI-MS m/z calc. 318.19434, found 319.3 (M+1)⁺; Retention time: 1.42 minutes (LC method A).

Stage 2: The product from above was taken up in MeOH (10 mL), cooled to 0° C. and treated with sodium borohydride (80.4 mg, 2.125 mmol) in portions. The reaction was warmed to room temperature and stirred for 1 hour and then treated with saturated ammonium chloride (10 mL). The product was extracted with ethyl acetate (15 ml×2), washed with water (10 mL), brine (10 mL), dried over anhydrous sodium sulfate, filtered, and concentrated in vacuo. The crude residue was purified by flash column chromatography (12 g silica). The compound was eluted with ethyl acetate/hexanes (0-70%) over 30 minutes to give tert-butyl 4-benzyl-3-(1-hydroxyethyl)piperazine-1-carboxylate (267.3 mg, 76%) ESI-MS m/z calc. 320.21, found 321.2 (M+1)⁺; Retention time: 0.92 minutes (LC method A).

### Step 3: 3-[[4-[1-(4-tert-Butoxycarbonylpiperazin-2-yl)ethoxy]-6-(2,6-dimethylphenyl)pyrimidin-2-yl]sulfamoyl]benzoic acid

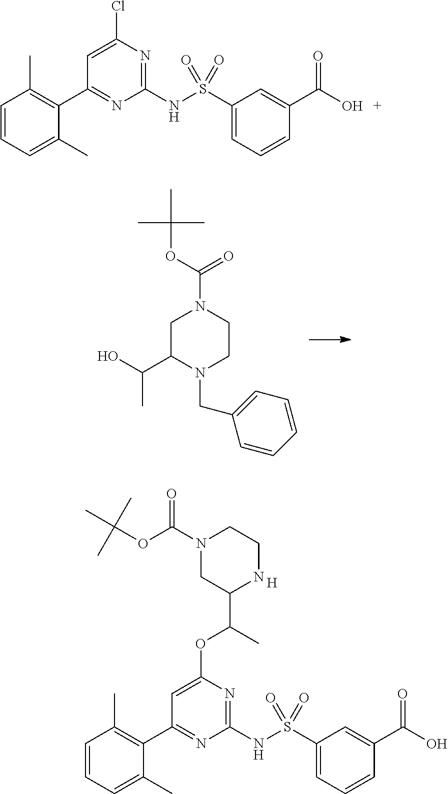

Stage 1: A THE (0.5 mL) mixture of tert-butyl 4-benzyl-3-(1-hydroxyethyl)piperazine-1-carboxylate (188.4 mg, 0.5880 mmol), 3-[[4-chloro-6-(2,6-dimethylphenyl)pyrimidin-2-yl]sulfamoyl]benzoic acid (163.8 mg, 0.3920 mmol), and sodium tert-butoxide (172.3 mg, 1.793 mmol) was stirred at room temperature for 16 h. The solutions were filtered and the filtrate dissolved in 1.4 mL MeOH, and injected (5×400 uL injections) onto a Reverse-phase Reverse phase HPLC for chromatography using a 15 min gradient of 1-99% MeCN in water (HCl modifier) to give 3-[[4-[1-(1-benzyl-4-tert-butoxycarbonyl-piperazin-2-yl)ethoxy]-6-(2,6-dimethylphenyl)pyrimidin-2-yl]sulfamoyl]benzoic acid (hydrochloride salt) (101.4 mg, 35%) ESI-MS m/z calc. 701.2883, found 702.2 (M+1)⁺; Retention time: 1.39 minutes (LC method A).

Stage 2: The product was taken up in MeOH (3 mL) and sparged with nitrogen for 3 minutes and then treated with Pd(OH)₂ (19.7 mg, 0.02806 mmol). The system was evacuated and purged with nitrogen (3×) and then stirred under an atmosphere of Hydrogen (balloon) at room temperature for 2 hours and then filtered over a Celite pad. The filtrate was concentrated in vacuo to give 3-[[4-[1-(4-tert-butoxycarbonylpiperazin-2-yl)ethoxy]-6-(2,6-dimethylphenyl)pyrimidin-2-yl]sulfamoyl]benzoic acid (hydrochloride salt) (90.2 mg, 36%). ESI-MS m/z calc. 611.2414, found 612.3 (M+1)⁺; Retention time: 1.04 minutes (LC method A).

### Step 4: tert-Butyl 13-(2,6-dimethylphenyl)-9-methyl-2,17,17-trioxo-10-oxa-17λ⁶-thia-3,6,14,16,23-pentaazatetracyclo[16.3.1.111,15.03,8]tricosa-1(22),11,13,15(23),18,20-hexaene-6-carboxylate (Compound 173), and 13-(2,6-dimethylphenyl)-9-methyl-10-oxa-17λ⁶-thia-3,6,14,16,23-pentaazatetracyclo[16.3.1.111,15.03,8]tricosa-1(22),11,13,15(23),18,20-hexaene-2,17,17-trione (Compound 172)

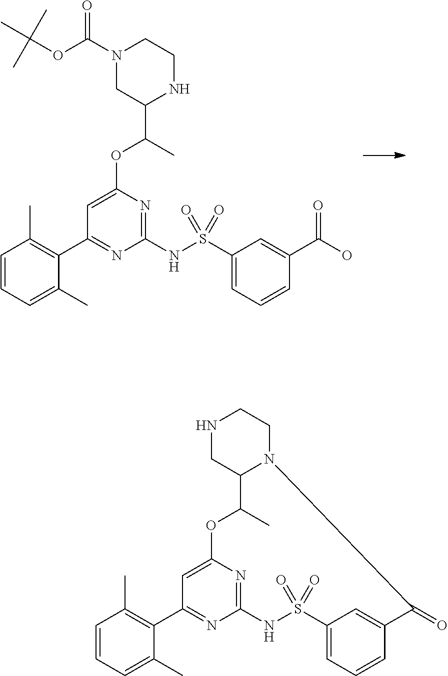 [SMILES: Cc1cccc(C)c1-c1cc(OC(C)C2CN(C(=O)OC(C)(C)C)CCN2)nc(NS(=O)(=O)c2cccc(C(=O)O)c2)n1 ; Cc1cccc(C)c1-c1cc2nc(n1)NS(=O)(=O)c1cccc(c1)C(=O)N1CCNCC1C(C)O2]

Stage 1: A DMF (5 mL) mixture of 3-[[4-[1-(4-tert-butoxycarbonylpiperazin-2-yl)ethoxy]-6-(2,6-dimethylphenyl)pyrimidin-2-yl]sulfamoyl]benzoic acid (hydrochloride salt) (35.2 mg, 0.05431 mmol) and HATU (34.2 mg, 0.08995 mmol) was stirred at room temperature and treated with DIPEA (50 μL, 0.2871 mmol). The mixture was stirred for 30 minutes and then quenched with 1 M HCl (10 mL) and diluted with ethyl acetate (40 mL). The organic layer was separated and washed with water (10 mL) and then brine (10 mL×2), dried over anhydrous sodium sulfate, filtered, and concentrated in vacuo. The crude residue was diluted with MeOH (1.5 mL) and purified by reverse phase HPLC using a 15 min gradient of 1-99% MeCN in water (HCl modifier) to give tert-butyl 13-(2,6-dimethylphenyl)-9-methyl-2,17,17-trioxo-10-oxa-17λ⁶-thia-3,6,14,16,23-pentaazatetracyclo[16.3.1.111,15.03,8]tricosa-1(22),11,13,15(23),18,20-hexaene-6-carboxylate (6.1 mg, 19%) ESI-MS m/z calc. 593.23083, found 594.3 (M+1)⁺; Retention time: 1.69 minutes (LC method A).

Stage 2: Half of the product from Stage 1 was treated with HCl (800 μL of 4 M, 3.200 mmol) (in dioxane) and stirred for 1 hour at room temperature. The mixture was concentrated in vacuo and dissolved in MeOH (1.5 mL) and purified by reverse phase HPLC using a 15 min gradient of 1-99% MeCN in water (HCl modifier) to give 13-(2,6-dimethylphenyl)-9-methyl-10-oxa-17λ⁶-thia-3,6,14,16,23-pentaazatetracyclo[16.3.1.111,15.03,8]tricosa-1(22),11,13,15(23),18,20-hexaene-2,17,17-trione (hydrochloride salt) (3.1 mg, 11%). ESI-MS m/z calc. 493.17838, found 494.4 (M+1)⁺; Retention time: 0.81 minutes (LC method A).

### Example 83: Preparation of Compound 174, Compound 175, and Compound 176

### Step 1: tert-Butyl 4-benzyl-3-(1-hydroxy-2-phenyl-ethyl)piperazine-1-carboxylate

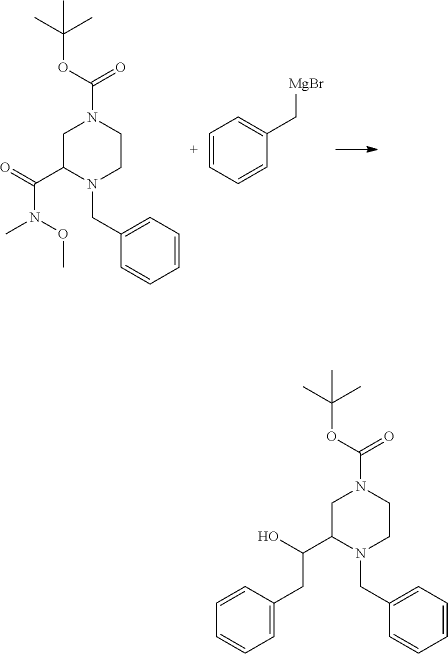

Stage 1: A THE (5 mL) mixture of tert-butyl 4-benzyl-3-[methoxy(methyl)carbamoyl]piperazine-1-carboxylate (404.3 mg, 1.112 mmol) was cooled to 0° C. and treated with a dropwise addition of benzylmagnesium bromide (2.5 mL of 0.9 M, 2.250 mmol). The reaction mixture was warmed to room temperature and stirred for 16 h. The reaction mixture was treated with an aqueous solution of saturated ammonium chloride (10 mL) and extracted with ethyl acetate (2×10 mL). The organic layer was dried over anhydrous sodium sulfate, filtered, and concentrated in vacuo to give tert-butyl 4-benzyl-3-(2-phenylacetyl)piperazine-1-carboxylate (139.6 mg, 32%) ESI-MS m/z calc. 394.22565, found 395.3 (M+1)⁺; Retention time: 1.49 minutes (LC method A).

Stage 2: The product from above was taken up in MeOH (10 mL), cooled to 0° C. and treated with sodium borohydride (64.7 mg, 1.710 mmol) in portions. The reaction was warmed to room temperature and stirred for 1 hour and then treated with saturated ammonium chloride (10 mL). The product was extracted with ethyl acetate (15 ml×2), washed with water (10 mL), brine (10 mL), dried over anhydrous sodium sulfate, filtered, and concentrated in vacuo. The crude residue was purified by flash column chromatography (12 g silica). The compound was eluted with ethyl acetate/hexanes (0-70%) over 30 minutes to give tert-butyl 4-benzyl-3-(1-hydroxy-2-phenyl-ethyl)piperazine-1-carboxylate (127.8 mg, 29%) ESI-MS m/z calc. 396.2413, found 397.3 (M+1)⁺; Retention time: 0.51 minutes (LC method A).

### Step 2: 3-[[4-[1-(4-tert-Butoxycarbonylpiperazin-2-yl)-2-phenyl-ethoxy]-6-(2,6-dimethylphenyl)pyrimidin-2-yl]sulfamoyl]benzoic acid

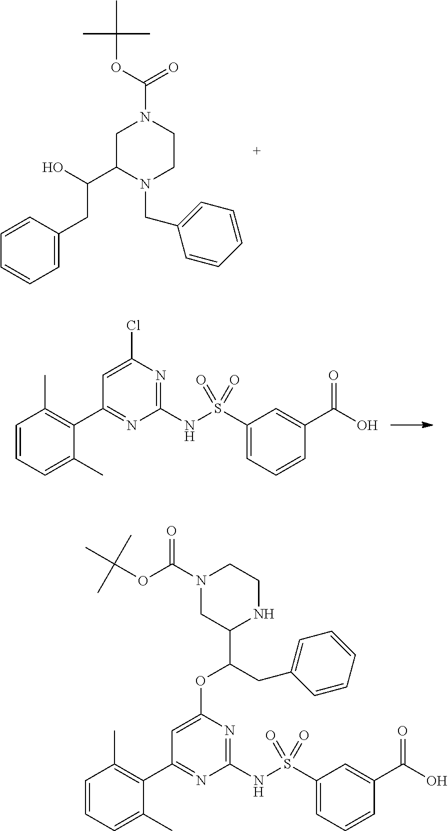 [SMILES: CC(C)(C)OC(=O)N1CCN(Cc2ccccc2)C(C(O)Cc2ccccc2)C1 ; Cc1cccc(C)c1-c1cc(Cl)nc(NS(=O)(=O)c2cccc(C(=O)O)c2)n1 ; Cc1cccc(C)c1-c1cc(OC(Cc2ccccc2)C2CN(C(=O)OC(C)(C)C)CCN2)nc(NS(=O)(=O)c2cccc(C(=O)O)c2)n1]

Stage 1: A THE (2 mL) mixture of tert-butyl 4-benzyl-3-(1-hydroxy-2-phenyl-ethyl)piperazine-1-carboxylate (143.4 mg, 0.3616 mmol), sodium tert-butoxide (157.2 mg, 1.636 mmol), and 3-[[4-chloro-6-(2,6-dimethylphenyl)pyrimidin-2-yl]sulfamoyl]benzoic acid (165.7 mg, 0.3965 mmol) was stirred at room temperature for 20 h. The solutions were filtered and the filtrate diluted with 0.5 mL MeOH, and purified by reverse phase HPLC using a 15 min gradient of 1-99% MeCN in water (HCl modifier) to give 3-[[4-[1-(1-benzyl-4-tert-butoxycarbonyl-piperazin-2-yl)-2-phenyl-ethoxy]-6-(2,6-dimethylphenyl)pyrimidin-2-yl]sulfamoyl]benzoic acid (hydrochloride salt) (192.1 mg, 65%) ESI-MS m/z calc. 777.31964, found 778.5 (M+1)⁺; Retention time: 1.68 minutes (LC method A).

Stage 2: The product was taken up in MeOH (5 mL) and purged with nitrogen for 5 minutes and then treated with dihydroxypalladium (99.3 mg, 0.1414 mmol). The system was evacuated and purged with nitrogen (3×) and then stirred under an atmosphere of hydrogen (balloon) for 4 h. The reaction mixture was filtered through a Celite bed and concentrated in vacuo to give 3-[[4-[1-(4-tert-butoxycarbonylpiperazin-2-yl)-2-phenyl-ethoxy]-6-(2,6-dimethylphenyl)pyrimidin-2-yl]sulfamoyl]benzoic acid (hydrochloride salt) (187.3 mg, 72%) ESI-MS m/z calc. 687.27264, found 688.4 (M+1)⁺; Retention time: 0.54 minutes (LC method D).

### Step 3: tert-butyl 9-benzyl-13-(2,6-dimethylphenyl)-2,17,17-trioxo-10-oxa-17λ⁶-thia-3,6,14,16,23-pentaazatetracyclo[16.3.1.111,15.03,8]tricosa-1(22),11,13,15(23),18,20-hexaene-6-carboxylate (Compound 175), 9-benzyl-13-(2,6-dimethylphenyl)-10-oxa-17λ⁶-thia-3,6,14,16,23-pentaazatetracyclo[16.3.1.111,15.03,8]tricosa-1(22),11,13,15(23),18,20-hexaene-2,17,17-trione (Compound 176), and 9-benzyl-13-(2,6-dimethylphenyl)-6-(propan-2-yl)-10-oxa-17λ⁶-thia-3,6,14,16,23-pentaazatetracyclo[16.3.1.111,15.03,8]tricosa-1(22),11,13,15(23),18,20-hexaene-2,17,17-trione (Compound 174)

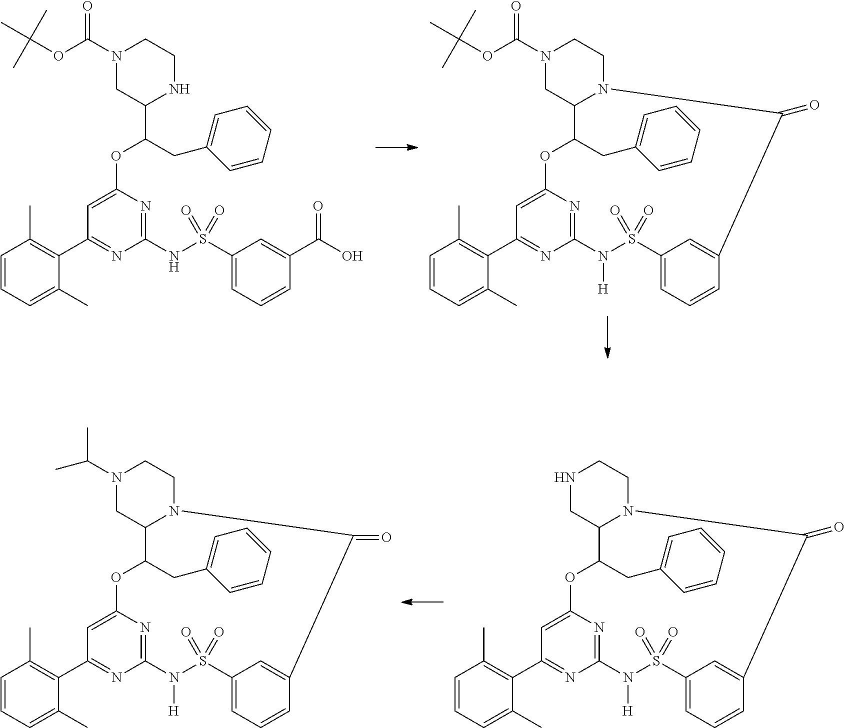 [SMILES: Cc1cccc(C)c1-c1cc(OC(Cc2ccccc2)C2CN(C(=O)OC(C)(C)C)CCN2)nc(NS(=O)(=O)c2cccc(C(=O)O)c2)n1 ; Cc1cccc(C)c1-c1cc2nc(n1)NS(=O)(=O)c1cccc(c1)C(=O)N1CCN(C(=O)OC(C)(C)C)CC1C(Cc1ccccc1)O2 ; Cc1cccc(C)c1-c1cc2nc(n1)NS(=O)(=O)c1cccc(c1)C(=O)N1CCN(C(C)C)CC1C(Cc1ccccc1)O2 ; Cc1cccc(C)c1-c1cc2nc(n1)NS(=O)(=O)c1cccc(c1)C(=O)N1CCNCC1C(Cc1ccccc1)O2]

Stage 1: A DMF (11 mL) solution of 3-[[4-[1-(4-tert-butoxycarbonylpiperazin-2-yl)-2-phenyl-ethoxy]-6-(2,6-dimethylphenyl)pyrimidin-2-yl]sulfamoyl]benzoic acid (hydrochloride salt) (86.4 mg, 0.1193 mmol), HATU (67.3 mg, 0.1770 mmol), and DIPEA (80 μL, 0.4593 mmol) was stirred at room temperature for 5 min and then concentrated in vacuo. The residue was taken up in MeOH (2 mL) and the solutions were filtered and the filtrate purified by reverse phase chromatography using a 15 min gradient of 1-99% MeCN in water (HCl modifier) to give tert-butyl 9-benzyl-13-(2,6-dimethylphenyl)-2,17,17-trioxo-10-oxa-17λ⁶-thia-3,6,14,16,23-pentaazatetracyclo[16.3.1.111,15.03,8]tricosa-1(22),11,13,15(23),18,20-hexaene-6-carboxylate (hydrochloride salt) (46.4 mg, 55%) ESI-MS m/z calc. 669.2621, found 670.4 (M+1)⁺; Retention time: 1.96 minutes (LC method A).

Stage 2: The product was taken up in HCl (3 mL of 4 M, 12.00 mmol) (in dioxane) and stirred at room temperature for 1 h and then concentrated in vacuo. The solution was filtered and the filtrate was dissolved in 1.8 mL MeOH, and purified by reverse phase HPLC using a 15 min gradient of 1-99% MeCN in water (HCl modifier) to give 9-benzyl-13-(2,6-dimethylphenyl)-10-oxa-17λ⁶-thia-3,6,14,16,23-pentaazatetracyclo[16.3.1.111,15.03,8]tricosa-1(22),11,13,15(23),18,20-hexaene-2,17,17-trione (hydrochloride salt) (4.9 mg, 7%) ESI-MS m/z calc. 569.20966, found 570.4 (M+1)⁺; Retention time: 1.14 minutes (LC method A).

Stage 3: The step 2 product was taken up in acetic acid (1 mL, 17.58 mmol) and treated with acetone (20 μL, 0.2724 mmol) followed by sodium triacetoxyborohydride (26.7 mg, 0.1260 mmol). The mixture was stirred at room temperature for 4 h. The solutions were filtered and the filtrate purified by reverse phase HPLC using a 15 min gradient of 1-99% MeCN in water (HCl modifier) to give 9-benzyl-13-(2,6-dimethylphenyl)-6-(propan-2-yl)-10-oxa-17λ⁶-thia-3,6,14,16,23-pentaazatetracyclo[16.3.1.111,15.03,8]tricosa-1(22),11,13,15(23),18,20-hexaene-2,17,17-trione (hydrochloride salt) (7.4 mg, 9%) ESI-MS m/z calc. 611.25665, found 612.4 (M+1)⁺; Retention time: 1.3 minutes (LC method A).

### Example 84: Preparation of Compound 177 and Compound 178

### Step 1: tert-Butyl 4-benzyl-3-(hydroxymethyl)piperazine-1-carboxylate

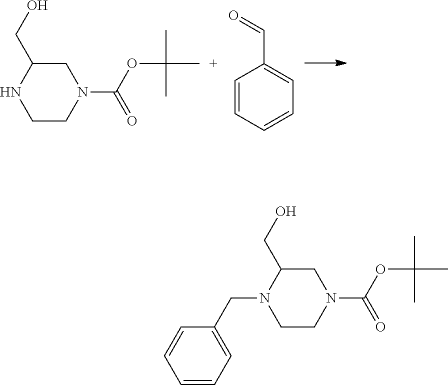

tert-butyl 3-(hydroxymethyl)piperazine-1-carboxylate (7 g, 32.37 mmol) and benzaldehyde (3.9 mL, 38.37 mmol) were combined in DCE (100 mL) with acetic acid (2.5 mL, 43.96 mmol), and stirred for 30 minutes at room temperature. The reaction mixture was then cooled to 0° C., and sodium triacetoxyborohydride (9 g, 42.46 mmol) was added. The reaction mixture was returned to room temperature and stirred for 16 hours. The reaction mixture was poured into aqueous sodium bicarbonate, and extracted 3× ethyl acetate. The combined organic layers were washed with brine, dried over sodium sulfate, and concentrated. The resulting crude was purified by chromatography on silica gel, eluting with a gradient of 0-100% ethyl acetate in dichloromethane to give tert-butyl 4-benzyl-3-(hydroxymethyl)piperazine-1-carboxylate (5.1 g, 51%) ESI-MS m/z calc. 306.19434, found 307.3 (M+1)⁺; Retention time: 0.35 minutes, LC method D.

### Step 2: 3-[[4-[(1-Benzyl-4-tert-butoxycarbonyl-piperazin-2-yl)methoxy]-6-(2,6-dimethylphenyl)pyrimidin-2-yl]sulfamoyl]benzoic acid

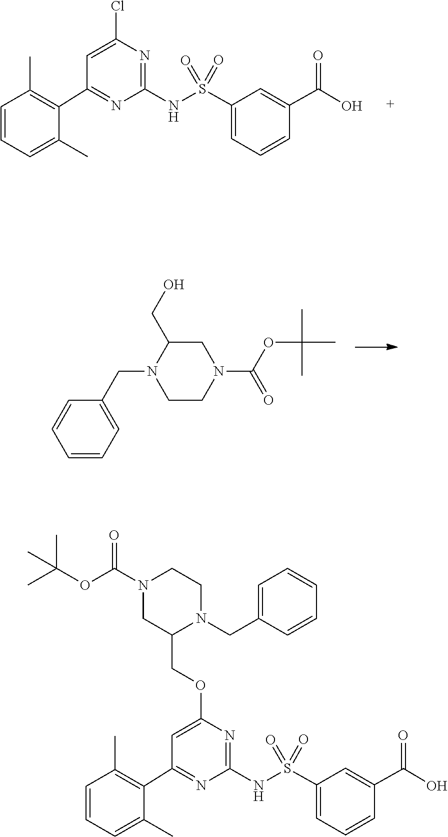

3-[[4-Chloro-6-(2,6-dimethylphenyl)pyrimidin-2-yl]sulfamoyl]benzoic acid (900 mg, 2.154 mmol) and tert-butyl 4-benzyl-3-(hydroxymethyl)piperazine-1-carboxylate (870 mg, 2.839 mmol) were combined in anhydrous NMP (12 mL). The reaction mixture was cooled to 0° C., and NaH (430 mg, 10.75 mmol) was slowly added in portions under a stream of nitrogen. The reaction mixture was then stirred at room temperature for 1 hour. After this time, the reaction mixture was slowly added to a flask containing aqueous ammonium chloride and ethyl acetate. The aqueous and organic layers were separated, and the aqueous was extracted an additional 4× ethyl acetate. The combined organics were washed with water, brine and dried over sodium sulfate. The resulting crude material was purified by chromatography on silica gel, eluting with 0-10% methanol in dichloromethane. Fractions containing product were combined and concentrated to give 3-[[4-[(1-benzyl-4-tert-butoxycarbonyl-piperazin-2-yl)methoxy]-6-(2,6-dimethylphenyl)pyrimidin-2-yl]sulfamoyl]benzoic acid (1.103 g, 74%) ESI-MS m/z calc. 687.27264, found 684.5 (M+1)⁺; Retention time: 0.54 minutes, LC method D.

### Step 3: 18-Benzyl-12-(2,6-dimethylphenyl)-15-oxa-8λ⁶-thia-1,9,11,18,22-pentaazatetracyclo[15.3.1.13,7.110,14]tricosa-3,5,7(23),10(22),11,13-hexaene-2,8,8-trione (Compound 178), and 12-(2,6-dimethylphenyl)-15-oxa-8λ⁶-thia-1,9,11,18,22-pentaazatetracyclo[15.3.1.13,7.110,14]tricosa-3(23),4,6,10(22),11,13-hexaene-2,8,8-trione

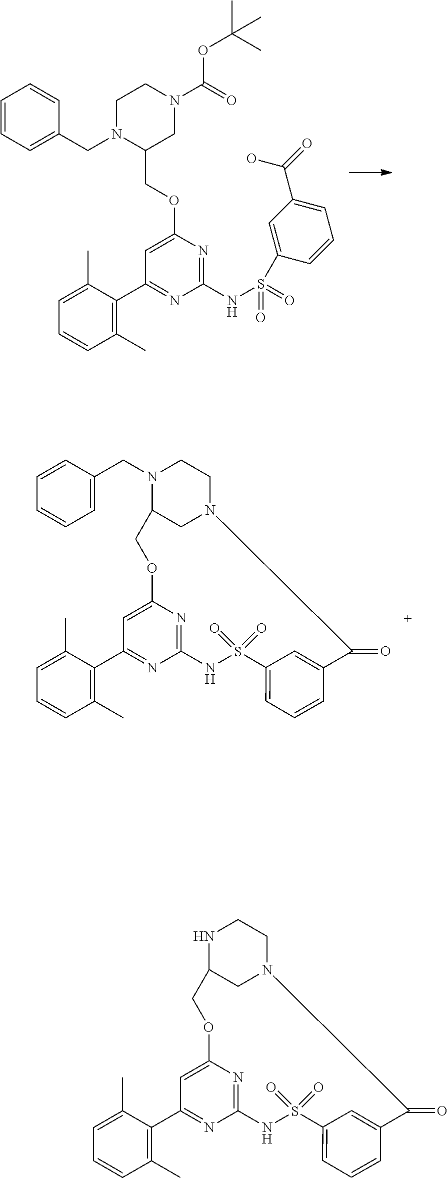 [SMILES: Cc1cccc(C)c1-c1cc(OCC2CN(C(=O)OC(C)(C)C)CCN2Cc2ccccc2)nc(NS(=O)(=O)c2cccc(C(=O)O)c2)n1 ; Cc1cccc(C)c1-c1cc2nc(n1)NS(=O)(=O)c1cccc(c1)C(=O)N1CCN(Cc3ccccc3)C(CO2)C1 ; Cc1cccc(C)c1-c1cc2nc(n1)NS(=O)(=O)c1cccc(c1)C(=O)N1CCNC(CO2)C1]

Stage 1: 3-[[4-[(1-benzyl-4-tert-butoxycarbonyl-piperazin-2-yl)methoxy]-6-(2,6-dimethylphenyl)pyrimidin-2-yl]sulfamoyl]benzoic acid (610 mg, 0.8869 mmol) was combined in dichloromethane (5 mL) with HCl (5 mL of 4 M, 20.00 mmol, in dioxane). The reaction mixture was stirred at room temperature for 90 minutes, then concentrated under vacuum. Hexanes were added, and the reaction mixture was concentrated a under vacuum a second time, to give 3-[[4-[(1-benzylpiperazin-2-yl)methoxy]-6-(2,6-dimethylphenyl)pyrimidin-2-yl]sulfamoyl]benzoic acid (Dihydrochloride salt) (590 mg, 101%) ESI-MS m/z calc. 587.2202, found 588.4 (M+1)⁺; Retention time: 0.46 minutes (LC method D).

Stage 2: The product was combined in DMF (70 mL) with HATU (440 mg, 1.157 mmol), and DIPEA (927 μL, 5.322 mmol) was added. The reaction mixture was stirred at room temperature for 1 hour, then diluted with water and ethyl acetate, and the layers were separated. The aqueous was extracted 2× with ethyl acetate, and the combined organics were then washed 2× with water, followed by brine. The organics were then dried over sodium sulfate, filtered, and concentrated. A significant amount of a dimeric side product was formed, and two silica gel columns (1-10% methanol in dichloromethane followed by 0-100% ethyl acetate in dichloromethane) to obtain 18-benzyl-12-(2,6-dimethylphenyl)-15-oxa-8λ⁶-thia-1,9,11,18,22-pentaazatetracyclo[15.3.1.13,7.110,14]tricosa-3(23),4,6,10(22),11,13-hexaene-2,8,8-trione (185 mg, 37%) ESI-MS m/z calc. 569.20966, found 570.3 (M+1)⁺; Retention time: 1.21 minutes (LC method A). A 12 mg portion of this product was further purified by reverse phase reverse phase HPLC (1-70% ACN in water, HCl modifier, 15 min run) to give 18-benzyl-12-(2,6-dimethylphenyl)-15-oxa-8λ⁶-thia-1,9,11,18,22-pentaazatetracyclo[15.3.1.13,7.110,14]tricosa-3,5,7(23),10(22),11,13-hexaene-2,8,8-trione (hydrochloride salt) (6 mg, 1%) ESI-MS m/z calc. 569.20966, found 570.3 (M+1)⁺; Retention time: 1.21 minutes (LC method A).

Stage 3: The main batch of product from stage 2 was dissolved in methanol (15 mL) in a nitrogen purged vial (sonication required). Dihydroxypalladium (80 mg, 0.1139 mmol) was added, and hydrogen gas was bubbled through the reaction mixture from a balloon for 15 minutes, after which the reaction was stirred at room temperature for 2 hours with the hydrogen balloon in place. The reaction vessel was then purged with nitrogen, filtered through Celite and concentrated to give as a white solid, 12-(2,6-dimethylphenyl)-15-oxa-8λ⁶-thia-1,9,11,18,22-pentaazatetracyclo[15.3.1.13,7.110,14]tricosa-3(23),4,6,10(22),11,13-hexaene-2,8,8-trione (94 mg, 22%) ESI-MS m/z calc. 479.16272, found 480.4 (M+1)⁺; Retention time: 0.32 minutes (LC method D).

### Step 4: 18-(3,3-Dimethylbutyl)-12-(2,6-dimethylphenyl)-15-oxa-8λ⁶-thia-1,9,11,18,22-pentaazatetracyclo[15.3.1.13,7.110,14]tricosa-3(23),4,6,10(22),11,13-hexaene-2,8,8-trione (Compound 177)

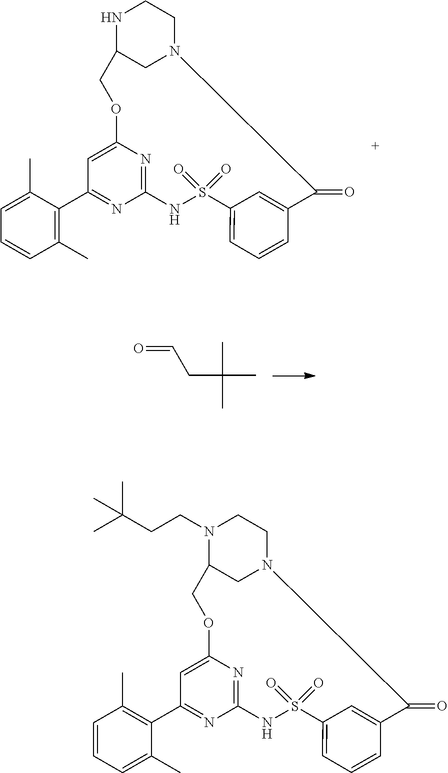

12-(2,6-Dimethylphenyl)-15-oxa-8λ⁶-thia-1,9,11,18,22-pentaazatetracyclo[15.3.1.13,7.110,14]tricosa-3(23),4,6,10(22),11,13-hexaene-2,8,8-trione (12 mg, 0.02502 mmol) was combined in acetic acid (0.5 mL) with 3,3-dimethylbutanal (13 mg, 0.1298 mmol). The reaction was stirred at room temperature for 5 minutes, then sodium triacetoxyborohydride (approximately 31.81 mg, 0.1501 mmol) was added and the reaction mixture was stirred at room temperature for 20 minutes. The reaction mixture was then diluted with 0.2 mL methanol, filtered, and purified by reverse phase HPLC (1-40% ACN or 1-70% ACN 15 min run with HCl modifier) to give 18-(3,3-dimethylbutyl)-12-(2,6-dimethylphenyl)-15-oxa-8λ⁶-thia-1,9,11,18,22-pentaazatetracyclo[15.3.1.13,7.110,14]tricosa-3(23),4,6,10(22),11,13-hexaene-2,8,8-trione (hydrochloride salt) (6 mg, 38%). ESI-MS m/z calc. 563.25665, found 564.4 (M+1)⁺; Retention time: 1.16 minutes; LC method A.

### Example 85: Preparation of Compound 179, Compound 180, and Compound 181

### Step 1: 2-[(4R)-2,2-Dimethyl-1,3-dioxolan-4-yl]acetaldehyde

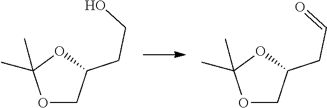

Pyridinium chlorochromate (16 g, 74.227 mmol) was added slowly to a suspension of activated molecular sieves (4 Å, 16 g) in dry dichloromethane (150 mL). The commercially available 2-[(4R)-2,2-dimethyl-1,3-dioxolan-4-yl]ethanol (3 g, 20.522 mmol) was dissolved in dichloromethane (15 mL) and added to the above mixture which was stirred at rt for 2 hours. The mixture was then diluted using diethylether (150 mL), filtered through silica gel and concentrated under reduced pressure, to give of the pure 2-[(4R)-2,2-dimethyl-1,3-dioxolan-4-yl]acetaldehyde (2.05 g, 69%) as a colorless oil; ¹H NMR (300 MHz, CDCl₃) δ 9.84-9.78 (m, 1H), 4.65-4.42 (m, 1H), 4.25-4.10 (m, 1H), 3.64-3.54 (m, 1H), 2.94-2.78 (m, 1H), 2.72-2.58 (m, 1H), 1.42 (s, 3H), 1.37 (s, 3H).

### Step 2: Methyl (E)-4-[(4R)-2,2-dimethyl-1,3-dioxolan-4-yl]but-2-enoate

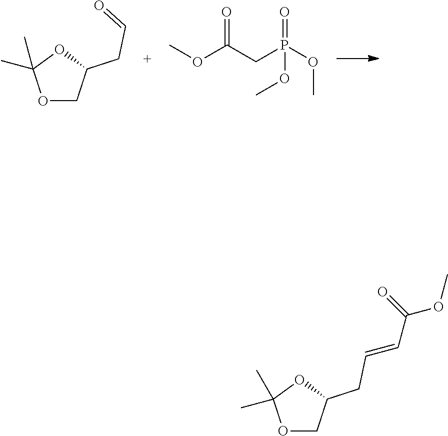

Methyl 2-dimethoxyphosphorylacetate (3.3750 g, 3 mL, 18.533 mmol) was added dropwise to a suspension of sodium hydride (830 mg, 20.752 mmol) in tetrahydrofuran (35 mL) at 0° C. under nitrogen, and the mixture of white slurry was stirred vigorously at 0° C. for 30 minutes. Then a solution of 2-[(4R)-2,2-dimethyl-1,3-dioxolan-4-yl]acetaldehyde (2.3 g, 15.954 mmol) in tetrahydrofuran (23 mL) was added dropwise at 0° C. and the whole was stirred for 2 hours at room temperature. The mixture was diluted with water (25 mL) and then was extracted with diethyl ether (50 mL). The resulting organic phase was washed brine (50 mL) and dried over sodium sulfate. The crude was purified by column chromatography (80 g, dichloromethane/diethyl ether: 3/7) to provide methyl (E)-4-[(4R)-2,2-dimethyl-1,3-dioxolan-4-yl]but-2-enoate (2.8 g, 88%) as a colorless oil; ¹H NMR (300 MHz, CDCl₃) δ 6.95 (dt, J=15.7, 7.1 Hz, 1H), 6.00-5.84 (m, 1H), 4.32-4.12 (m, 1H), 4.11-4.00 (m, 1H), 3.74 (s, 3H), 3.59 (dd, J=8.2, 6.8 Hz, 1H), 2.60-2.37 (m, 2H), 1.43 (s, 3H), 1.36 (s, 3H). ESI-MS m/z calc. 200.1049, found 201.2 (M+1)⁺; Retention time: 1.69 minutes, LC method K.

### Step 3: Methyl 4-[(4R)-2,2-dimethyl-1,3-dioxolan-4-yl]butanoate

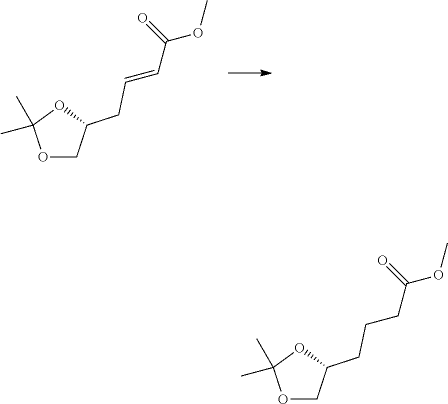

Methyl (E)-4-[(4R)-2,2-dimethyl-1,3-dioxolan-4-yl]but-2-enoate (3 g, 14.983 mmol) was dissolved in ethyl acetate (75 mL) and then 10% palladium on carbon (800 mg, 0.7517 mmol) was added. The resulting mixture was bubbled with hydrogen over 15 minutes and then was stirred at room temperature under balloon of hydrogen for 2 hours. LCMS showed complete conversion of starting material. The crude was filtered through Celite, washed with ethyl acetate (75 mL) and concentrated under reduced pressure to provide methyl 4-[(4R)-2,2-dimethyl-1,3-dioxolan-4-yl]butanoate (2.9 g, 96%) as a colorless oil; ¹H NMR (300 MHz, CDCl₃) δ 4.27-3.87 (m, 2H), 3.68 (s, 3H), 3.53 (t, J=6.9 Hz, 1H), 2.51-2.26 (m, 2H), 1.84-1.52 (m, 4H), 1.41 (s, 3H), 1.36 (s, 3H).

### Step 4: (6R)-6-(Hydroxymethyl)tetrahydropyran-2-one

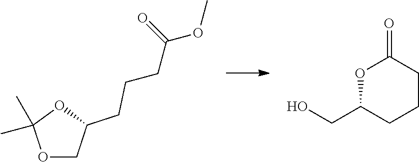

Activated 4 Å, molecular sieves (5 g), Amberlyst 15 Hydrogen resin (5 g) were added to a solution of methyl 4-[(4R)-2,2-dimethyl-1,3-dioxolan-4-yl]butanoate (3.5 g, 17.305 mmol) in acetonitrile (339.99 mL). The mixture was stirred vigorously at room temperature for 48 hours. The reaction was monitored by TLC. The mixture was then filtered and evaporated under reduced pressure. The residue was chromatographed on silica gel (40 g; eluting with EtOAc) to give (6R)-6-(hydroxymethyl)tetrahydropyran-2-one (1.15 g, 51%) as a colorless oil; ¹H NMR (300 MHz, CDCl₃) δ 4.49-4.36 (m, 1H), 3.89-3.75 (m, 1H), 3.73-3.62 (m, 1H), 2.71-2.56 (m, 1H), 2.55-2.35 (m, 1H), 2.17-2.06 (m, 1H), 2.05-1.85 (m, 3H), 1.80-1.65 (m, 1H).

### Step 5: [(2R)-6-Oxotetrahydropyran-2-yl]methyl methanesulfonate

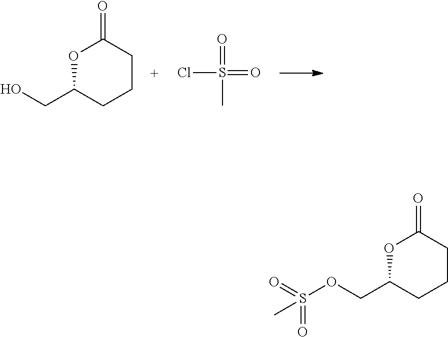

To a solution of (6R)-6-(hydroxymethyl)tetrahydropyran-2-one (1.1 g, 8.4523 mmol) in dichloromethane (33 mL) was added triethylamine (1.7424 g, 2.4 mL, 17.219 mmol) at room temperature and methanesulfonyl chloride (1.1840 g, 0.8 mL, 10.336 mmol) at room temperature and then the resulting mixture was stirred at that temperature for 1 hour. The resulting mixture was diluted with dichloromethane (50 mL) and quenched with a saturated aqueous solution of NaCl (50 mL). The aqueous layer was extracted with dichloromethane (2×50 mL). The combined organic layer was dried over sodium sulfate, filtered and concentrated under reduced pressure. Purification by silica gel column chromatography (40 g, heptanes/ethyl acetate=1:3) gave [(2R)-6-oxotetrahydropyran-2-yl]methyl methanesulfonate (1.5 g, 85%) as a colorless oil; ¹H NMR (300 MHz, CDCl₃) δ 4.71-4.51 (m, 1H), 4.44-4.26 (m, 2H), 3.11 (s, 3H), 2.74-2.41 (m, 2H), 2.11-1.89 (m, 3H), 1.84-1.66 (m, 1H).

### Step 6: (6R)-6-(Azidomethyl)tetrahydropyran-2-one

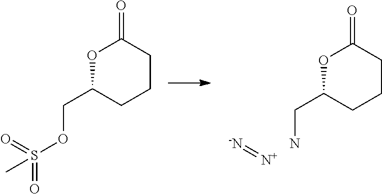

To a solution of [(2R)-6-oxotetrahydropyran-2-yl]methyl methanesulfonate (1.5 g, 7.2035 mmol) in dimethylformamide (30 mL) was added azidosodium (700 mg, 10.768 mmol) at room temperature, and then the resulting mixture was stirred at 90° C. for 2 hours. The reaction was quenched with water (50 mL) at 0° C. The aqueous layer was extracted with diethyl ether (2×50 mL). The combined organic layer was dried over sodium sulfate, filtered and concentrated under reduced pressure. Purification by silica gel column chromatography (40 g, Heptane/EtOAc=1:3) gave (6R)-6-(azidomethyl)tetrahydropyran-2-one (800 mg, 72%) as a colorless oil; ¹H NMR (300 MHz, CDCl₃) δ 4.53-4.38 (m, 1H), 3.59-3.40 (m, 2H), 2.73-2.57 (m, 1H), 2.56-2.35 (m, 1H), 2.08-1.83 (m, 3H), 1.81-1.62 (m, 1H).

### Step 7: (6R)-6-(Azidomethyl)tetrahydropyran-2-ol

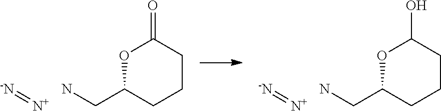

To a solution (−70° C.) of (6R)-6-(azidomethyl)tetrahydropyran-2-one (800 mg, 5.1561 mmol) in tetrahydrofuran (8 mL) was added slowly a solution of Diisobutylaluminum hydride (5.5 mL of 1 M, 5.5000 mmol) in hexanes. The mixture was stirred for 45 minutes at −70° C. An additional portion of a solution of Diisobutylaluminum hydride (1.6 mL of 1 M, 1.6000 mmol) in hexanes was added and the mixture was stirred at −60° C. for at least 6 hours. Then it was quenched by the addition of water (15 mL) at −60° C. with vigorous stirring. The mixture was allowed to reach room temperature and 0.5 M HCl (25 mL) and dichloromethane (75 mL) were added. The organic layer was separated, dried over sodium sulfate, filtered and evaporated. The residue was purified by column chromatography (40 g ethyl acetate) to provide (6R)-6-(azidomethyl)tetrahydropyran-2-ol (710 mg, 88%) as clear oil.

### Step 8: (3R)-7-Isobutylazepan-3-ol

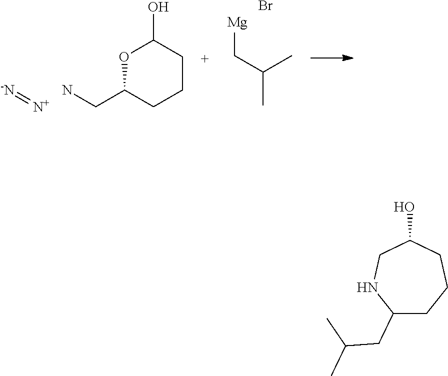

Trimethylphosphine (9 mL of 1 M, 9.0000 mmol) in toluene was added to a solution of the (6R)-6-(azidomethyl)tetrahydropyran-2-ol (700 mg, 4.4538 mmol) in methanol (14 mL) under nitrogen atmosphere. Upon consumption of the starting material (detected by TLC, eluent: heptane/EtOAc=1/1, v/v) and formation of the imine intermediate (detected by TLC, eluent: EtOAc/MeOH=10/1, v/v) the reaction mixture was concentrated under reduced pressure and the residue was co-evaporated twice with toluene (15 mL). The product was collected in a mixture of anhydrous tetrahydrofuran (14 mL) and toluene (14 mL) and then bromo(isobutyl)magnesium (12 mL of 2 M, 24.000 mmol) in diethyl ether was added at 0° C. under nitrogen atmosphere. Upon consumption of the imine intermediate the reaction was quenched with ammonium chloride saturated (50 mL). The aqueous layer was extracted with diethyl ether (3×50 mL), the combined organic phases were dried over sodium sulfate, filtered and concentrated in vacuo. The crude was dissolved in methanolic HCl (3 mL of 3 M, 9.0000 mmol) and then stirred for 2 hours and then concentrated under vacuum to gave (3R)-7-isobutylazepan-3-ol (hydrochloride salt) (800 mg, 86%) as brownish solid; ESI-MS m/z calc. 171.1623, found 172.2 (M+1)⁺; Retention time: 1.25 minutes, LC method K.

### Step 9: N-[4-Chloro-6-(2,6-dimethylphenyl)pyrimidin-2-yl]-3-[(6R)-6-hydroxy-2-isobutyl-azepane-1-carbonyl]benzenesulfonamide

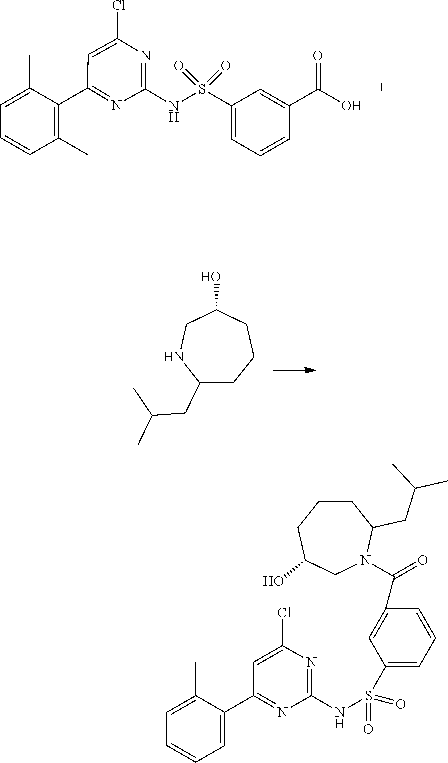 [SMILES: Cc1cccc(C)c1-c1cc(Cl)nc(NS(=O)(=O)c2cccc(C(=O)O)c2)n1 ; CC(C)CC1CCC[C@@H](O)CN1 ; Cc1ccccc1-c1cc(Cl)nc(NS(=O)(=O)c2cccc(C(=O)N3C[C@H](O)CCCC3CC(C)C)c2)n1]

3-[[4-chloro-6-(2,6-dimethylphenyl)pyrimidin-2-yl]sulfamoyl]benzoic acid (550 mg, 1.3162 mmol) was dissolved in dimethylformamide (22 mL). The mixture was bubbled with nitrogen for 15 min and then HATU (600 mg, 1.5780 mmol) and triethylamine (435.60 mg, 0.6 mL, 4.3048 mmol) were added followed by (3R)-7-isobutylazepan-3-ol (hydrochloride salt) (300 mg, 1.4441 mmol). The resulting mixture was stirred at room temperature overnight under nitrogen. The reaction mixture was diluted with diethyl ether (50 mL) and washed with aqueous HCl (1 M, 2×50 mL) and brine (2×50 mL). The organic layer was dried over sodium sulfate, filtered and concentrated under reduced pressure to give N-[4-chloro-6-(2,6-dimethylphenyl)pyrimidin-2-yl]-3-[(6R)-6-hydroxy-2-isobutyl-azepane-1-carbonyl]benzenesulfonamide (650 mg, 86%) as off-white foaming solid; ESI-MS m/z calc. 570.2068, found 571.2 (M+1)⁺; Retention time: 2.12 minutes, LC method K.

### Step 10: (16R)-12-(2,6-Dimethylphenyl)-20-(2-methylpropyl)-15-oxa-8λ⁶-thia-1,9,11,22-tetraazatetracyclo[14.4.1.13,7.110,14]tricosa-3(23),4,6,10(22),11,13-hexaene-2,8,8-trione (Compound 181)

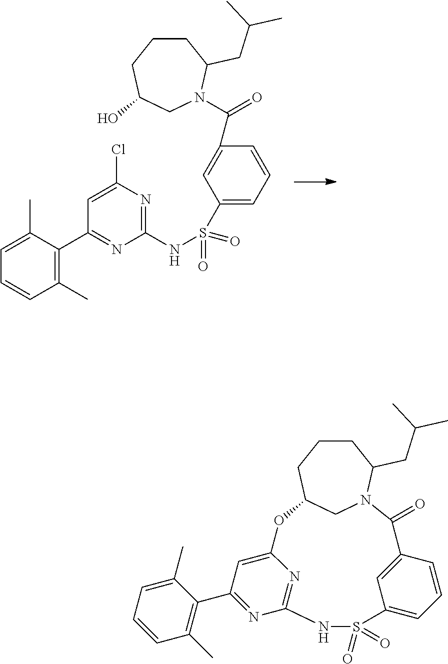 [SMILES: Cc1cccc(C)c1-c1cc(Cl)nc(NS(=O)(=O)c2cccc(C(=O)N3C[C@H](O)CCCC3CC(C)C)c2)n1 ; Cc1cccc(C)c1-c1cc2nc(n1)NS(=O)(=O)c1cccc(c1)C(=O)N1C[C@@H](CCCC1CC(C)C)O2]

N-[4-Chloro-6-(2,6-dimethylphenyl)pyrimidin-2-yl]-3-[(6R)-6-hydroxy-2-isobutyl-azepane-1-carbonyl]benzenesulfonamide (950 mg, 1.6634 mmol) was dissolved in tetrahydrofuran (95 mL) and then sodium tert-butoxide (960 mg, 9.9892 mmol) was added. The resulting mixture was stirred at room temperature under nitrogen for 24 hours. The reaction mixture was diluted with EtOAc (50 mL) and washed with aqueous HCl (1 M, 1×25 mL) and brine (2×25 mL). The organic layer was dried over sodium sulfate, filtered and concentrated under reduced pressure. The crude product was chromatographed on a 40 gram column silica gel, eluting with EtOAc/hexane (9/1) to provide 110 mg of desired product (87% purity by LCMS) and then the resulting product was purified by preparative HPLC to provide (16R)-12-(2,6-dimethylphenyl)-20-(2-methylpropyl)-15-oxa-8λ⁶-thia-1,9,11,22-tetraazatetracyclo[14.4.1.13,7.110,14]tricosa-3(23),4,6,10(22),11,13-hexaene-2,8,8-trione (40 mg, 4%) as white solid; ¹H NMR (300 MHz, DMSO-d₆) δ 8.40 (br. s., 1H), 7.82 (d, J=6.5 Hz, 1H), 7.60 (t, J=7.5 Hz, 1H), 7.53-7.42 (m, 1H), 7.31-7.19 (m, 1H), 7.16-7.06 (m, 2H), 6.29-6.03 (m, 1H), 5.60-5.32 (m, 1H), 4.65-4.38 (m, 1H), 3.28-3.17 (m, 1H), 3.10-2.95 (m, 1H), 2.33-2.20 (m, 2H), 2.13-1.92 (m, 6H), 1.91-1.70 (m, 2H), 1.67-1.28 (m, 6H), 0.93 (d, J 6.5 Hz, 6H). ESI-MS m/z calc. 534.2301, found 535.3 (M+1)⁺; Retention time: 3.34 minutes, LC method U.

### Step 11: (16R)-12-(2,6-Dimethylphenyl)-20-(2-methylpropyl)-15-oxa-8λ⁶-thia-1,9,11,22-tetraazatetracyclo[14.4.1.13,7.110,14]tricosa-3(23),4,6,10(22),11,13-hexaene-2,8,8-trione, diastereomer 1 (Compound 180), and (16R)-12-(2,6-dimethylphenyl)-20-(2-methylpropyl)-15-oxa-8λ⁶-thia-1,9,11,22-tetraazatetracyclo[14.4.1.13,7.110,14]tricosa-3(23),4,6,10(22),11,13-hexaene-2,8,8-trione, diastereomer 2 (Compound 179)

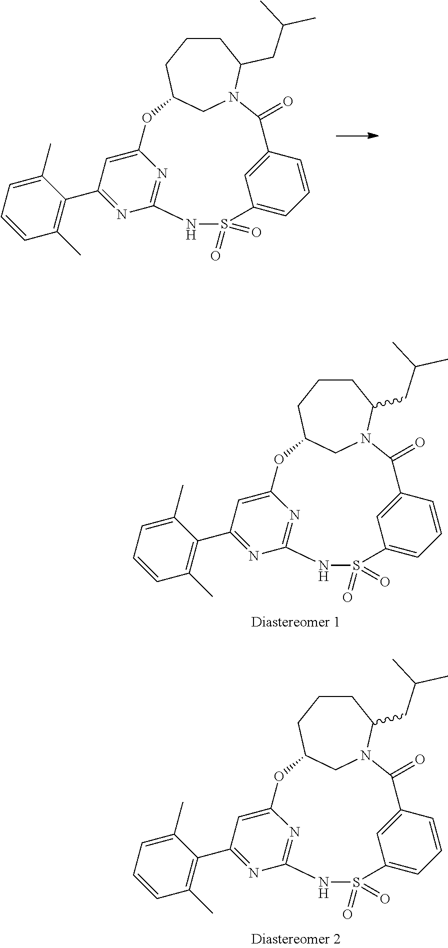 [SMILES: Cc1cccc(C)c1-c1cc2nc(n1)NS(=O)(=O)c1cccc(c1)C(=O)N1C[C@@H](CCCC1CC(C)C)O2 ; Cc1cccc(C)c1-c1cc2nc(n1)NS(=O)(=O)c1cccc(c1)C(=O)N1C[C@@H](CCCC1CC(C)C)O2 ; Cc1cccc(C)c1-c1cc2nc(n1)NS(=O)(=O)c1cccc(c1)C(=O)N1C[C@@H](CCCC1CC(C)C)O2]

(31R)-16-(2,6-Dimethylphenyl)-37-isobutyl-2-oxa-6-thia-7-aza-3(3,1)-azepana-1(4,2)-pyrimidina-5(1,3)-benzenacycloheptaphan-4-one 6,6-dioxide (36.1 mg, 0.06752 mmol) (mixture of diastereomers) was separated using a normal phase SFC-MS method using a AS-H column (250×21.2 mm, 5 μm particle size) sold by Chiral Technologies (pn: 20945), and a dual gradient run from 5-40% mobile phase B over 17.5 minutes. Mobile phase A=CO2. Mobile phase B=MeOH (20 mM NH₃). Flow rate=5-15% MeOH [20 mM NH3] 80 mL/min, 15-80% MeOH [20 mM NH3] 40 mL/min. injection volume=variable, and column temperature=40° C. to give: first to elute, diastereomer 1, (16R)-12-(2,6-dimethylphenyl)-20-(2-methylpropyl)-15-oxa-8λ⁶-thia-1,9,11,22-tetraazatetracyclo[14.4.1.13,7.110,14]tricosa-3(23),4,6,10(22),11,13-hexaene-2,8,8-trione (17.1 mg, 95%). ¹H NMR (500 MHz, DMSO-d₆) δ 12.90 (s, 1H), 8.42 (s, 1H), 7.82 (s, 1H), 7.61 (s, 1H), 7.49 (s, 1H), 7.26 (s, 1H), 7.11 (s, 2H), 6.16 (s, 1H), 5.50 (s, 1H), 4.52 (s, 1H), 3.26 (s, 1H), 3.05 (d, J=16.6 Hz, 1H), 2.25 (s, 2H), 2.03 (s, 6H), 1.77 (s, 1H), 1.61 (s, 2H), 1.45 (s, 2H), 1.36 (s, 2H), 1.20 (d, J=44.8 Hz, 1H), 0.94 (d, J=6.3 Hz, 6H). ESI-MS m/z calc. 534.2301, found 535.0 (M+1)⁺; Retention time: 1.95 minutes (LC method A); and second to elute, diastereomer 2,2, (16R)-12-(2,6-dimethylphenyl)-20-(2-methylpropyl)-15-oxa-8λ⁶-thia-1,9,11,22-tetraazatetracyclo[14.4.1.13,7.110,14]tricosa-3(23),4,6,10(22),11,13-hexaene-2,8,8-trione (6.5 mg, 36%) ¹H NMR (500 MHz, DMSO-d₆) δ 8.41 (s, 1H), 7.84 (s, 1H), 7.61 (s, 1H), 7.50 (s, 1H), 7.26 (s, 1H), 7.13 (s, 2H), 6.17 (s, 1H), 5.50 (s, 1H), 4.52 (s, 1H), 3.25 (s, 1H), 3.06 (s, 1H), 2.25 (s, 2H), 2.02 (s, 6H), 1.80 (s, 1H), 1.58 (s, 2H), 1.45 (s, 2H), 1.36 (s, 2H), 1.25 (s, 1H), 1.16 (s, 1H), 0.94 (s, 6H). ESI-MS m/z calc. 534.2301, found 535.0 (M+1)⁺; Retention time: 1.97 minutes (3 min run) (LC method A).

### Example 86: Preparation of Compound 182 and Compound 183

### Step 1: tert-Butyl 2-[(3-tert-butylphenyl)methylamino]acetate

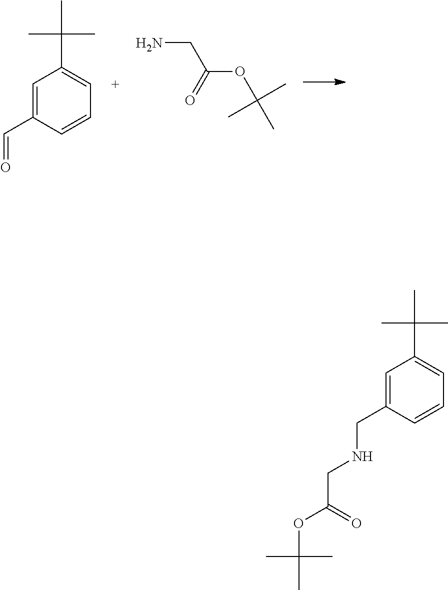

3-tert-Butylbenzaldehyde (3.1742 g, 19.566 mmol) was dissolved in DCE (100 mL), tert-butyl 2-aminoacetate (hydrochloride salt) (3.28 g, 19.566 mmol) and TEA (2.1779 g, 2.9999 mL, 21.523 mmol) were added and the mixture was stirred for 30 min at room temperature. Sodium triacetoxyborohydride (6.4275 g, 30.327 mmol) was added and the mixture was stirred at room temperature for 24 hours. The mixture was quenched with saturated potassium carbonate (50 mL) and layers were separated. The aqueous layer was washed with chloroform (2×50 mL), organic fractions were combined, dried over sodium sulfate and evaporated, the residue was purified by silica gel column chromatography using 0-20% hexanes-ethyl acetate to give tert-butyl 2-[(3-tert-butylphenyl)methylamino]acetate (3.753 g, 62%) as a yellow oil. ESI-MS m/z calc. 277.2042, found 278.2 (M+1)⁺; Retention time: 2.49 minutes, LC method T.

### Step 2: tert-Butyl 2-[(3-tert-butylphenyl)methyl-(2,2,2-trifluoroacetyl)amino]acetate

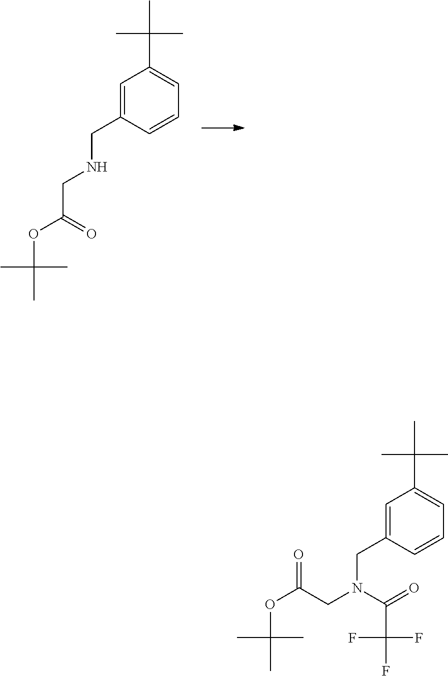 [SMILES: CC(C)(C)OC(=O)CNCc1cccc(C(C)(C)C)c1 ; CC(C)(C)OC(=O)CN(Cc1cccc(C(C)(C)C)c1)C(=O)C(F)(F)F]

To a stirring solution of tert-butyl 2-[(3-tert-butylphenyl)methylamino]acetate (3.753 g, 13.529 mmol) and TEA (2.7380 g, 3.7713 mL, 27.058 mmol) in DCM (50 mL) trifluoroacetic anhydride (3.1257 g, 2.0686 mL, 14.882 mmol) was added dropwise and the mixture was stirred at room temperature for 16 hours. Saturated ammonium chloride (50 mL) was added, aqueous phase was separated and extracted with chloroform (2×20 mL). The organic fractions were combined, dried over sodium sulfate and evaporated, and the residue was purified by silica gel column chromatography using 0-10% hexanes-ethyl acetate to give tert-butyl 2-[(3-tert-butylphenyl)methyl-(2,2,2-trifluoroacetyl)amino]acetate (4.82 g, 86%) as a colorless oil. ¹H NMR (250 MHz, CDCl₃) δ 7.48-7.12 (m, 3H), 7.03 (d, J=7.1 Hz, 1H), 4.72 (d, J=6.8 Hz, 2H), 4.01-3.71 (m, 2H), 1.46 (s, 9H), 1.32 (s, 9H).

### Step 3: 2-[(3-tert-Butylphenyl)methyl-(2,2,2-trifluoroacetyl)amino]acetic acid

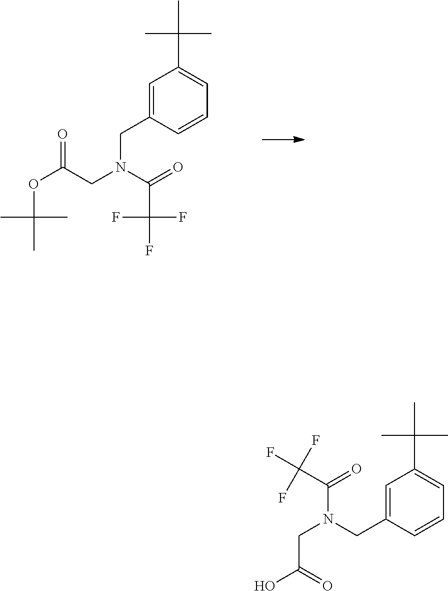 [SMILES: CC(C)(C)OC(=O)CN(Cc1cccc(C(C)(C)C)c1)C(=O)C(F)(F)F ; CC(C)(C)c1cccc(CN(CC(=O)O)C(=O)C(F)(F)F)c1]

tert-Butyl 2-[(3-tert-butylphenyl)methyl-(2,2,2-trifluoroacetyl)amino]acetate (4.82 g, 12.908 mmol) was dissolved in a mixture of TFA (37.000 g, 25 mL, 324.50 mmol) and DCM (25 mL) and the mixture was stirred at room temperature for 2 hours. Then it was evaporated to give 2-[(3-tert-butylphenyl)methyl-(2,2,2-trifluoroacetyl)amino]acetic acid (3.83 g, 85%) as a white solid. ESI-MS m/z calc. 317.1239, found 318.1 (M+1)⁺; Retention time: 3.14 minutes, LC method T.

### Step 4: 2-[(3-tert-Butylphenyl)methyl-(2,2,2-trifluoroacetyl)amino]acetyl chloride

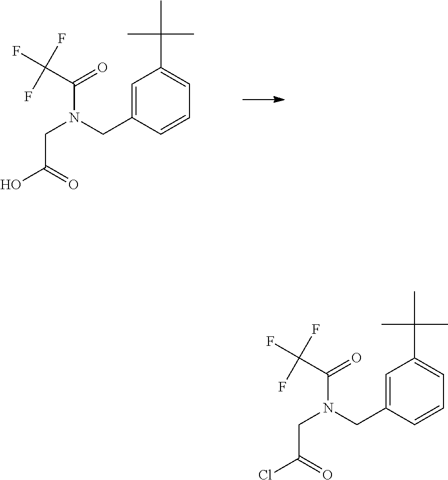 [SMILES: CC(C)(C)c1cccc(CN(CC(=O)O)C(=O)C(F)(F)F)c1 ; CC(C)(C)c1cccc(CN(CC(=O)Cl)C(=O)C(F)(F)F)c1]

2-[(3-tert-Butylphenyl)methyl-(2,2,2-trifluoroacetyl)amino]acetic acid (4.3 g, 13.552 mmol) was dissolved in DCM (80 mL) under argon and thionyl chloride (2.4184 g, 1.4746 mL, 20.328 mmol) was added. The mixture was refluxed for 5 hours and evaporated in vacuo to give crude 2-[(3-tert-butylphenyl)methyl-(2,2,2-trifluoroacetyl)amino]acetyl chloride (4.5 g, 89%) as an off-white solid. ¹H NMR (250 MHz, CDCl₃) δ 7.55-7.10 (m, 3H), 7.10-6.85 (m, 1H), 4.71 (s, 2H), 4.39 (s, 2H), 1.32 (s, 9H).

### Step 5: 7-tert-Butyl-2-(2,2,2-trifluoroacetyl)-1,3-dihydroisoquinolin-4-one

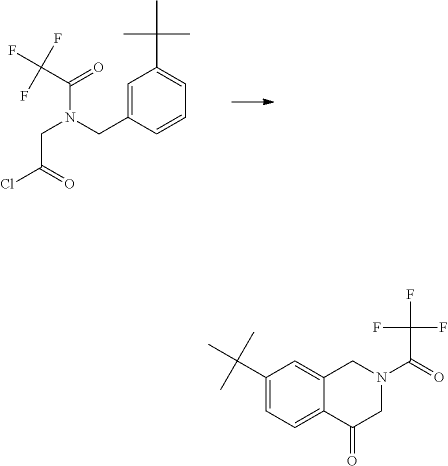 [SMILES: CC(C)(C)c1cccc(CN(CC(=O)Cl)C(=O)C(F)(F)F)c1 ; CC(C)(C)c1ccc2c(c1)CN(C(=O)C(F)(F)F)CC2=O]

2-[(3-tert-Butylphenyl)methyl-(2,2,2-trifluoroacetyl)amino]acetyl chloride (4.5 g, 13.403 mmol) was dissolved in DCM (150 mL) under argon, the solution was cooled to −78° C. and AlCl₃ (6.2550 g, 46.910 mmol) was added in one portion. The mixture was allowed to slowly warm up to −10° C., then cooled to −30° C. and HCl (100 mL of 3 M, 300.00 mmol) was added dropwise. The mixture was allowed to warm up to 0° C., the layers were separated and the aqueous layer was extracted with DCM (2×50 mL). The organic fractions were combined, dried over sodium sulfate and evaporated to give crude 7-tert-butyl-2-(2,2,2-trifluoroacetyl)-1,3-dihydroisoquinolin-4-one (4 g, 94%) that was used without further purification. ESI-MS m/z calc. 299.1133, found 300.0 (M+1)⁺; Retention time: 3.27 minutes, LC method T.

### Step 6: 7-tert-Butyl-1,2,3,4-tetrahydroisoquinolin-4-ol

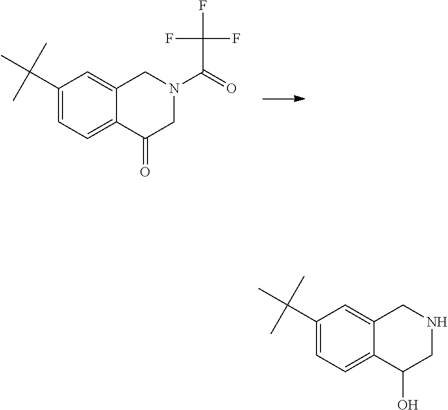 [SMILES: CC(C)(C)c1ccc2c(c1)CN(C(=O)C(F)(F)F)CC2=O ; CC(C)(C)c1ccc2c(c1)CNCC2O]

7-tert-Butyl-2-(2,2,2-trifluoroacetyl)-1,3-dihydroisoquinolin-4-one (4 g, 13.365 mmol) was dissolved in ethanol (100 mL) and sodium borohydride (1.5169 g, 1.6052 mL, 40.095 mmol) was added portionwise. The mixture was stirred at room temperature for 3 hours, saturated sodium bicarbonate (50 mL) was added and the mixture was extracted with chloroform (3×50 mL). The organic fractions were combined, evaporated and the residue was purified by silica gel column chromatography using 0-10% dichloromethane-methanol to give 7-tert-butyl-1,2,3,4-tetrahydroisoquinolin-4-ol (2.513 g, 87%) as a colorless oil. ESI-MS m/z calc. 205.1467, found 206.2 (M+1)⁺; Retention time: 1.93 minutes, LC method T.

### Step 7: tert-Butyl 7-tert-butyl-4-hydroxy-3,4-dihydro-1H-isoquinoline-2-carboxylate

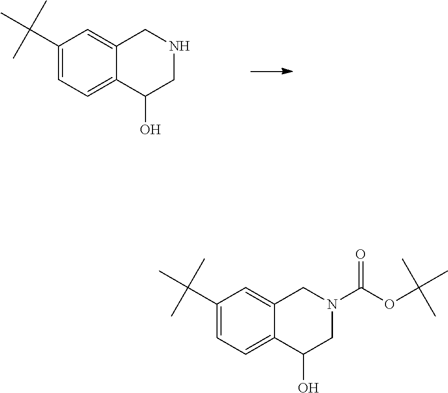 [SMILES: CC(C)(C)c1ccc2c(c1)CNCC2O ; CC(C)(C)OC(=O)N1Cc2cc(C(C)(C)C)ccc2C(O)C1]

7-tert-Butyl-1,2,3,4-tetrahydroisoquinolin-4-ol (2.513 g, 12.241 mmol) was dissolved in DCM (100 mL), NaOH (8.7412 g, 50 mL of 15% w/w, 32.782 mmol) was added followed by tert-butoxycarbonyl tert-butyl carbonate (2.6716 g, 2.8122 mL, 12.241 mmol). The mixture was vigorously stirred for 2 hours, phases were separated, and the aqueous layer was extracted with DCM (2×20 mL). The organic fractions were combined, dried over sodium sulfate end evaporated, the residue was purified by silica gel column chromatography using 0-20% hexanes-ethyl acetate to give tert-butyl 7-tert-butyl-4-hydroxy-3,4-dihydro-1H-isoquinoline-2-carboxylate (3.045 g, 78%). ESI-MS m/z calc. 305.1991, found 306.3 (M+1)⁺; Retention time: 2.65 minutes. ¹H NMR (250 MHz, DMSO-d₆) δ 7.35 (d, J=8.1 Hz, 1H), 7.26 (d, J=8.1 Hz, 1H), 7.17 (s, 1H), 5.41 (d, J=5.8 Hz, 1H), 4.52 (d, J=13.9 Hz, 3H), 3.72 (dd, J=12.8, 4.4 Hz, 1H), 1.43 (s, 9H), 1.27 (s, 9H). LC method T.

### Step 8: 20-tert-Butyl-12-(2,6-dimethylphenyl)-15-oxa-8λ⁶-thia-1,9,11,25-tetraazapentacyclo[14.7.1.13,7.110,14.017,22]hexacosa-3,5,7(26),10,12,14(25),17,19,21-nonaene-2,8,8-trione

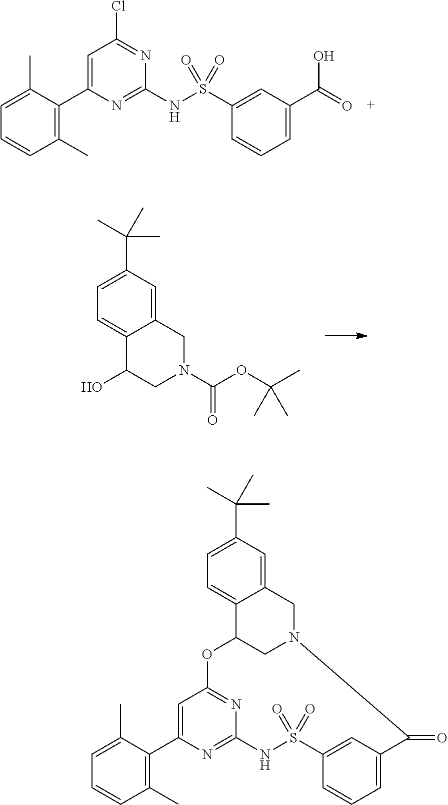 [SMILES: Cc1cccc(C)c1-c1cc(Cl)nc(NS(=O)(=O)c2cccc(C(=O)O)c2)n1 ; CC(C)(C)OC(=O)N1Cc2cc(C(C)(C)C)ccc2C(O)C1 ; Cc1cccc(C)c1-c1cc2nc(n1)NS(=O)(=O)c1cccc(c1)C(=O)N1Cc3cc(C(C)(C)C)ccc3C(C1)O2]

In a 20-mL vial, tert-butyl 7-tert-butyl-4-hydroxy-3,4-dihydro-1H-isoquinoline-2-carboxylate (353.8 mg, 1.111 mmol) was dissolved in dioxane (3.0 mL), to which a dioxane solution of HCl (3.0 mL of 4.0 M, 12.00 mmol) was added. This mixture was stirred at room temperature for 1 h, then at 70° C. for 1 h. This mixture was then cooled to room temperature, after which it was evaporated to dryness in vacuo to give a yellow solid. This intermediate was carried onto the next step without further purification. In a 20-mL vial, the product was mixed with THE (3.0 mL), to which NaOtBu (498.3 mg, 5.185 mmol) as added. This mixture was stirred at room temperature for 10 min, after which it was cooled to 0° C. Then, 3-[[4-chloro-6-(2,6-dimethylphenyl)pyrimidin-2-yl]sulfamoyl]benzoic acid (550 mg, 1.316 mmol) was added, and the resulting mixture was stirred at 0° C. for 30 min, and at room temperature for 30 min. In a separate 20-mL vial, a solution of HATU (845.2 mg, 2.223 mmol) in DMF (6.0 mL) was prepared. The above-prepared substrate mixture was added dropwise onto this HATU solution, and the resulting mixture was stirred at room temperature for 15 min. This mixture was then quenched with 1 N HCl solution (30 mL) and diluted with ethyl acetate (120 mL). The layers were separated, and the organic layer was washed with 1 N HCl solution (40 mL), water (40 mL) and saturated aqueous sodium chloride solution (40 mL), then dried over sodium sulfate, filtered, and evaporated in vacuo to give ˜600 mg of a yellow solid. Purification by silica gel chromatography (24 g of silica column) using a gradient eluent of 1 to 70% ethyl acetate in hexanes gave 20-tert-butyl-12-(2,6-dimethylphenyl)-15-oxa-8λ⁶-thia-1,9,11,25-tetraazapentacyclo[14.7.1.13,7.110,14.017,22]hexacosa-3,5,7(26),10,12,14(25),17,19,21-nonaene-2,8,8-trione (90.7 mg, 14%) ESI-MS m/z calc. 568.2144, found 569.5 (M+1)⁺; Retention time: 1.97 minutes, LC method A.

### Step 9: 20-tert-Butyl-12-(2,6-dimethylphenyl)-15-oxa-8λ⁶-thia-1,9,11,25-tetraazapentacyclo[14.7.1.13,7.110,14.017,22]hexacosa-3,5,7(26),10,12,14(25),17,19,21-nonaene-2,8,8-trione, peak 1 (Compound 182), and 20-tert-butyl-12-(2,6-dimethylphenyl)-15-oxa-8λ⁶-thia-1,9,11,25-tetraazapentacyclo[14.7.1.13,7.110,14.017,22]hexacosa-3,5,7(26),10,12,14(25),17,19,21-nonaene-2,8,8-trione, peak 2 (Compound 183)

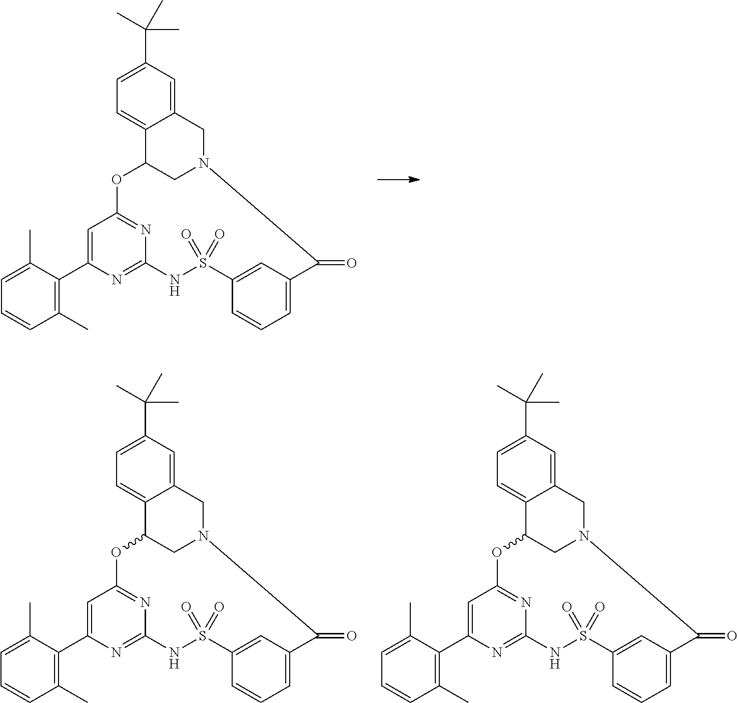

In a 3-mL vial, 20-tert-butyl-12-(2,6-dimethylphenyl)-15-oxa-8λ⁶-thia-1,9,11,25-tetraazapentacyclo[14.7.1.13,7.110,14.017,22]hexacosa-3,5,7(26),10,12,14(25),17,19,21-nonaene-2,8,8-trione (90.7 mg, 0.1595 mmol) was dissolved in a mixture of MeCN (2.5 mL) and DMSO (2.5 mL) to achieve a concentration of ca. 20 mg/mL. Separation of the enantiomers was achieved with an SFC purification method using a (R,R)-Whelk-O column (250×10 mm, 5 μm particle size) at 40° C., with a mobile phase of 34% MeCN:MeOH (+20 mM NH₃)⁺66% CO₂, a flow rate of 70 mL/min, an injection volume of 500 μL, and a pressure of 100 bar. The collected batches were labeled “Peak 1” (27 mg) and “Peak 2” (18 mg). These products were separately re-purified by reverse phase preparative chromatography using a C₁₈ column and a gradient eluent of 1 to 99% acetonitrile in water containing 5 mM hydrochloric acid to give: Peak 1, 20-tert-butyl-12-(2,6-dimethylphenyl)-15-oxa-8λ⁶-thia-1,9,11,25-tetraazapentacyclo[14.7.1.13,7.110,14.017,22]hexacosa-3,5,7(26),10,12,14(25),17,19,21-nonaene-2,8,8-trione (19.6 mg, 22%) ESI-MS m/z calc. 568.2144, found 569.5 (M+1)⁺; Retention time: 1.97 minutes (LC method A); 3.04 minutes (chiral RR 5-min method); 2.08 minutes (chiral AS-3 5-min method); 2.79 minutes (chiral LUX-4 5-min method); ¹H NMR (400 MHz, DMSO-d₆) δ 13.45-11.78 (broad d, 1H), 8.58 (s, 1H), 8.05-7.92 (m, 1H), 7.87-7.62 (m, 2H), 7.53 (d, J=8.3 Hz, 1H), 7.43 (d, J=1.9 Hz, 1H), 7.39 (dd, J=8.2, 2.0 Hz, 1H), 7.27 (t, J=7.6 Hz, 1H), 7.14 (d, J=7.6 Hz, 2H), 6.68-6.32 (m, 2H), 5.28 (d, J=17.3 Hz, 1H), 4.44 (d, J=17.3 Hz, 1H), 4.09 (dd, J=13.0, 4.4 Hz, 1H), 3.13 (dd, J=13.0, 10.7 Hz, 1H), 2.25-1.99 (bs, 6H), 1.31 (s, 9H); and peak 2, 20-tert-butyl-12-(2,6-dimethylphenyl)-15-oxa-8λ⁶-thia-1,9,11,25-tetraazapentacyclo[14.7.1.13,7.110,14.017,22]hexacosa-3,5,7(26),10,12,14(25),17,19,21-nonaene-2,8,8-trione (12.4 mg, 14%). ESI-MS m/z calc. 568.2144, found 569.5 (M+1)⁺; Retention time: 1.97 minutes (LC method A); 3.25 minutes (chiral RR 5-min method); 2.33 minutes (chiral AS-3 5-min method); 3.12 minutes (chiral LUX-4 5-min method); ¹H NMR (400 MHz, DMSO-d₆) δ 13.71-11.61 (broad d, 1H), 8.58 (s, 1H), 8.05-7.92 (m, 1H), 7.86-7.65 (m, 2H), 7.53 (d, J=8.2 Hz, 1H), 7.43 (d, J=1.9 Hz, 1H), 7.39 (dd, J=8.2, 2.0 Hz, 1H), 7.27 (t, J=7.6 Hz, 1H), 7.14 (d, J=7.6 Hz, 2H), 6.65-6.31 (m, 2H), 5.28 (d, J=17.4 Hz, 1H), 4.44 (d, J=17.3 Hz, 1H), 4.09 (dd, J=13.2, 4.6 Hz, 1H), 3.13 (dd, J=13.0, 10.7 Hz, 1H), 2.25-1.99 (bs, 6H), 1.31 (s, 9H).

### Example 87: Preparation of Compound 184 and Compound 185

### Step 1: 1-Bromo-4-tert-butyl-2-iodo-benzene

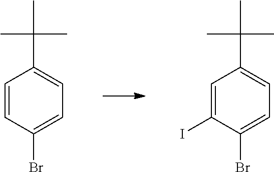

To a solution of 1-bromo-4-tert-butyl-benzene (100 g, 469.23 mmol) in TFA (1.5000 L) was added NIS (110.85 g, 492.69 mmol) portion-wise at room temperature. The reaction was allowed to stir for 4 hours before the volatiles were removed under reduced pressure. The crude residue was diluted with water (500 mL) and EtOAc (500 mL). The aqueous layer was extracted three times with EtOAc (3×1 L). The combined organic layers were washed with sodium bicarbonate (1 L), water (1 L), and brine (1 L), then dried over sodium sulfate and concentrated under vacuum. This crude residue was dissolved in hexanes and passed through a pad of silica gel. The pad of silica gel was washed three times with hexanes (3×500 mL), this residue was concentrated to give 1-bromo-4-tert-butyl-2-iodo-benzene (158.86 g, 98%). ¹H NMR (250 MHz, CDCl₃) δ 7.84 (s, 1H), 7.58-7.40 (m, 1H), 7.29-7.08 (m, 1H), 1.28 (s, 9H).

### Step 2: tert-Butyl N-[(1R)-1-[methoxy(methyl)carbamoyl]-3-methyl-butyl]carbamate

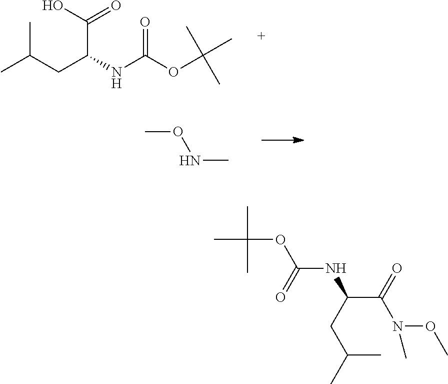

(2R)-2-(tert-Butoxycarbonylamino)-4-methyl-pentanoic acid (20 g, 86.472 mmol) EDC (24.866 g, 129.71 mmol) and HOBt (17.527 g, 129.71 mmol) were dissolved in DCM (200 mL) and cooled to 0° C. Next, N-methoxymethanamine (7.9231 g, 129.71 mmol) and DIPEA (22.351 g, 30.123 mL, 172.94 mmol) were added. The reaction was allowed to stir at room temperature overnight and was quenched with water (100 mL). The layers were separated and the aqueous layer was extracted three times with DCM (50 mL) and the combined organic layers were dried over sodium sulfate and concentrated. The residue was purified by flash column chromatography using 0-50% hexanes:diethylether as an eluent to give tert-butyl N-[(1R)-1-[methoxy(methyl)carbamoyl]-3-methyl-butyl]carbamate (15.62 g, 63%) as a colorless oil. ESI-MS m/z calc. 274.1893, found 275.3 (M+1)⁺; Retention time: 3.04 minutes, LC method T.

### Step 3: 2-Bromo-5-tert-butyl-benzaldehyde

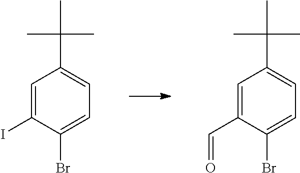

To a solution of 1-bromo-4-tert-butyl-2-iodo-benzene (235.93 g, 695.94 mmol) in dry THE (2 L) was added i-PrMgBr (1.6 L of 1 M, 1.6000 mol) dropwise at −78° C. The solution was stirred at this temperature for 4.5 hours. DMF (203.48 g, 215.55 mL, 2.7838 mol) was added to the solution at −78° C. and the reaction was stirred for 2 hours at this temperature before being allowed to warm to room temperature overnight. The solution was quenched with water (1 L) and the aqueous layer separated and extracted with diethyl ether (3×1 L). The organic layers were washed with brine (1 L) and dried over sodium sulfate. The organic residue was purified by silica gel chromatography eluting 0-2% hexanes-diethyl ether to give 2-bromo-5-tert-butyl-benzaldehyde (122.09 g, 73%). ¹H NMR (250 MHz, CDCl₃) δ 10.36 (s, 1H), 7.94 (d, J=2.5 Hz, 1H), 7.57 (d, J=8.4 Hz, 1H), 7.50 (d, J=2.5 Hz, 1H), 1.33 (s, 9H). ESI-MS m/z calc. 240.01498, Retention time: 3.34 minutes; LC method T.

### Step 4: tert-Butyl N-[(1R)-1-formyl-3-methyl-butyl]carbamate

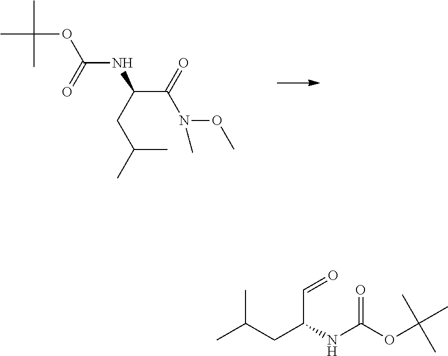 [SMILES: CON(C)C(=O)[C@@H](CC(C)C)NC(=O)OC(C)(C)C ; CC(C)C[C@H](C=O)NC(=O)OC(C)(C)C]

tert-Butyl N-[(1R)-1-[methoxy(methyl)carbamoyl]-3-methyl-butyl]carbamate (31.881 g, 116.20 mmol) was dissolved in THE (750 mL) and cooled to 0° C. Next, LAH (2.6462 g, 69.720 mmol) was added slowly. The reaction was stirred at 0° C. for 2 hours then quenched with 200 mL saturated Rochelle's salt solution. The reaction was allowed to stir overnight until the mixture became biphasic and the aqueous layer was slightly cloudy. The layers were separated and the aqueous layer extracted twice with diethylether (200 mL). The combined organic layers were dried over sodium sulfate and concentrated. The crude residue was dry loaded on silica gel and purified by flash column chromatography (0-15% hexanes:diethylether). Analysis of the fractions by TLC (KMnO4 stain) revealed the appropriate factions to collect to give tert-butyl N-[(1R)-1-formyl-3-methyl-butyl]carbamate (15 g, 48%) as a colorless oil. ¹H NMR (250 MHz, CDCl₃) δ 9.58 (s, 1H), 5.02-4.87 (m, 1H), 4.33-4.12 (m, 1H), 1.92-1.54 (m, 3H), 1.44 (s, 9H), 0.96 (dd, J=6.5, 1.5 Hz, 6H).

### Step 5: 2-(2-Bromo-5-tert-butyl-phenyl)-1,3-dioxolane

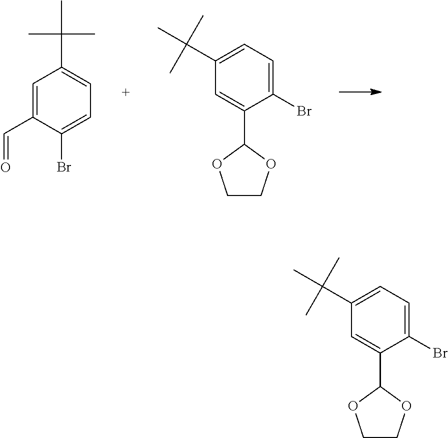 [SMILES: CC(C)(C)c1ccc(Br)c(C=O)c1 ; CC(C)(C)c1ccc(Br)c(C2OCCO2)c1 ; CC(C)(C)c1ccc(Br)c(C2OCCO2)c1]

To a solution of 2-bromo-5-tert-butyl-benzaldehyde (49.29 g, 204.42 mmol) in EtOH (492.90 mL) was added sodium borohydride (9.2803 g, 9.8204 mL, 245.30 mmol) at 0° C. The reaction was stirred at this temperature for 1 hour before being quenched with slow addition of water. The solution was concentrated in vacuum to remove solvent before being extracted with DCM (3×300 mL). The combined organic layers were washed with brine (500 mL) before being dried over sodium sulfate and concentrated. The organic residue was purified by silica gel chromatography eluting 0-4% hexanes-diethyl ether to give 2-(2-bromo-5-tert-butyl-phenyl)-1,3-dioxolane (26.68 g, 46%) as a yellow oil ESI-MS m/z calc. 284.0412, found 285.0 (M+1)⁺; Retention time: 3.32 minutes. ¹H NMR (250 MHz, CDCl₃) δ 7.61 (d, J=2.6 Hz, 1H), 7.48 (d, J=8.4 Hz, 1H), 7.35-7.09 (m, 1H), 6.07 (s, 1H), 4.38-4.13 (m, 2H), 4.13-3.99 (m, 2H), 1.31 (s, 9H). ESI-MS m/z calc. 284.0412, found 285.0 (M+1)⁺; Retention time: 3.32 minutes; LC method T.

### Step 6: tert-butyl N-[(1R)-1-[(R)-[4-tert-butyl-2-(1,3-dioxolan-2-yl)phenyl]-hydroxy-methyl]-3-methyl-butyl]carbamate, and tert-butyl N-[(1R)-1-[(S)-[4-tert-butyl-2-(1,3-dioxolan-2-yl)phenyl]-hydroxy-methyl]-3-methyl-butyl]carbamate

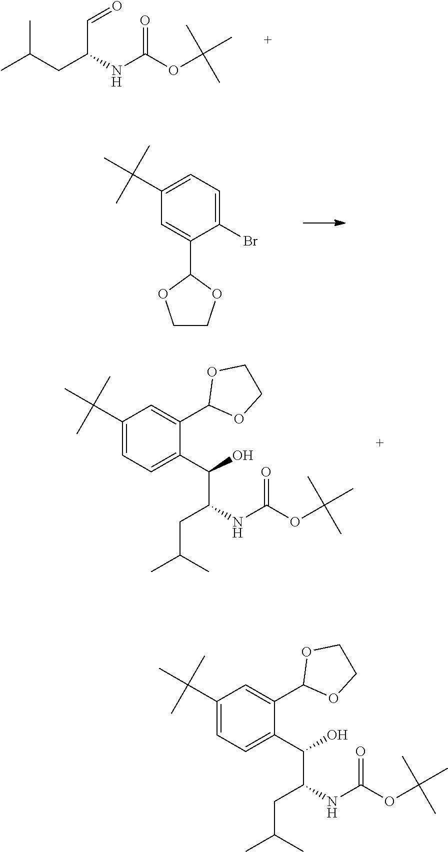

2-(2-Bromo-5-tert-butyl-phenyl)-1,3-dioxolane (26.6 g, 93.275 mmol) was dissolved in THE (80 mL) and magnesium (2.7205 g, 111.93 mmol) was added. The reaction was refluxed for 5 hours. The reaction was then cooled to room temperature and stirred overnight. The dark brown mixture was cooled to 0° C. and cannulated into a solution of tert-butyl N-[(1R)-1-formyl-3-methyl-butyl]carbamate (8.0324 g, 37.31 mmol) in THE (80 mL) which was cooled to 0° C. The reaction was allowed to stir for 2 hours and was cooled to 0° C. and quenched with ammonium chloride (150 mL). The layers were separated, and the aqueous layer was extracted twice with diethylether (100 mL). The combined organic layers were washed with water (50 mL) and brine (50 mL), dried over magnesium sulfate and concentrated. The crude residue was dry loaded on to silica gel and purified by flash column chromatography using 0-50% hexanes:diethylether as an eluent to give two products: tert-Butyl N-[(1R)-1-[(R)-[4-tert-butyl-2-(1,3-dioxolan-2-yl)phenyl]-hydroxy-methyl]-3-methyl-butyl]carbamate (3.57 g, 20%) was collected as a yellow foam. ¹H NMR (250 MHz, CDCl₃) δ 7.63-7.33 (m, 3H), 5.96 (s, 1H), 4.99 (s, 1H), 4.78 (d, J=9.5 Hz, 1H), 4.23-3.94 (m, 4H), 1.62-1.44 (m, 2H), 1.30 (s, 18H), 0.97-0.84 (m, 6H) LCMS retention time: 3.90 minutes (LC method T) and tert-butyl N-[(1R)-1-[(S)-[4-tert-butyl-2-(1,3-dioxolan-2-yl)phenyl]-hydroxy-methyl]-3-methyl-butyl]carbamate (3.2 g, 17%), which was collected a yellow oil. ¹H NMR (250 MHz, CDCl₃) δ 7.60-7.38 (m, 3H), 6.01 (s, 1H), 5.20 (s, 1H), 5.03 (d, J=5.7 Hz, 1H), 4.83 (d, J=9.3 Hz, 1H), 4.26-3.95 (m, 4H), 1.65-1.43 (m, 2H), 1.30 (s, 18H), 1.03-0.78 (m, 6H), LCMS retention time: 3.90 minutes (LC method T).

### Step 7: tert-Butyl (3R,4S)-7-tert-butyl-4-hydroxy-3-isobutyl-3,4-dihydro-1H-isoquinoline-2-carboxylate

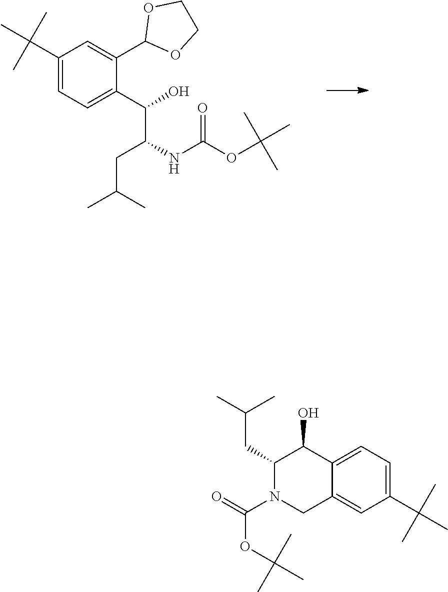

tert-Butyl N-[(1R)-1-[(S)-[4-tert-butyl-2-(1,3-dioxolan-2-yl)phenyl]-hydroxy-methyl]-3-methyl-butyl]carbamate (3.788 g, 8.9855 mmol) was dissolved in HCl (4M in dioxanes) (22.5 mL of 4 M, 90.000 mmol) and stirred for 1 h. The volatiles were removed in vacuo. The crude residue was dissolved in EtOH (70 mL) and cooled to 0° C. Then sodium triacetoxyborohydride (3.82 g, 18.024 mmol) was added to the reaction in portions. After 1 hour the volatiles were removed, and the reaction was diluted with ammonium chloride and EtOAc. The aqueous phase was extracted three times with EtOAc, dried over sodium sulfate and concentrated. The crude residue was dissolved in THE (70 mL) and 3.75M NaOH solution (70 mL). Boc anhydride (3.95 g, 18.099 mmol) was added and the reaction stirred for 1 h. The layers were separated, and the aqueous layer was extracted three times with EtOAc (20 mL). The combined organic layers were dried over sodium sulfate and concentrated. The crude residue was dry loaded on to silica gel and purified by flash column chromatography using 0-40% hexanes:diethylether as an eluent (220 nm monitor). The appropriate fractions (visualized by TLC/KMnO₄ stain) were collected to give tert-butyl (3R,4S)-7-tert-butyl-4-hydroxy-3-isobutyl-3,4-dihydro-1H-isoquinoline-2-carboxylate (668 mg, 20%) as a light yellow oil. ESI-MS m/z calc. 361.2617, found 362.4 (M+1)⁺; Retention time: 3.33 minutes, (LC method W). ¹H NMR (500 MHz, DMSO-d₆) δ 7.41 (d, J=8.0 Hz, 1H), 7.24 (d, J=8.2 Hz, 1H), 7.12 (d, J=9.7 Hz, 1H), 5.63 (d, J=5.4 Hz, 1H), 4.76 (dd, J=24.6, 17.5 Hz, 1H), 4.68-4.59 (m, 1H), 4.53-4.33 (m, 1H), 4.05 (dd, J=60.7, 17.6 Hz, 1H), 1.42 (s, 9H), 1.39 (dd, J=8.1, 4.5 Hz, 1H), 1.25 (s, 9H), 1.09-0.95 (m, 2H), 0.95-0.78 (m, 6H)

### Step 8: (16S,24R)-20-tert-Butyl-12-(2,6-dimethylphenyl)-24-(2-methylpropyl)-15-oxa-8λ⁶-thia-1,9,11,25-tetraazapentacyclo[14.7.1.13,7.110,14.017,22]hexacosa-3,5,7(26),10,12,14(25),17,19,21-nonaene-2,8,8-trione (Compound 184), and (16R,24R)-20-tert-butyl-12-(2,6-dimethylphenyl)-24-(2-methylpropyl)-15-oxa-8λ⁶-thia-1,9,11,25-tetraazapentacyclo[14.7.1.13,7.110,14.017,22]hexacosa-3,5,7(26),10,12,14(25),17,19,21-nonaene-2,8,8-trione (Compound 185)

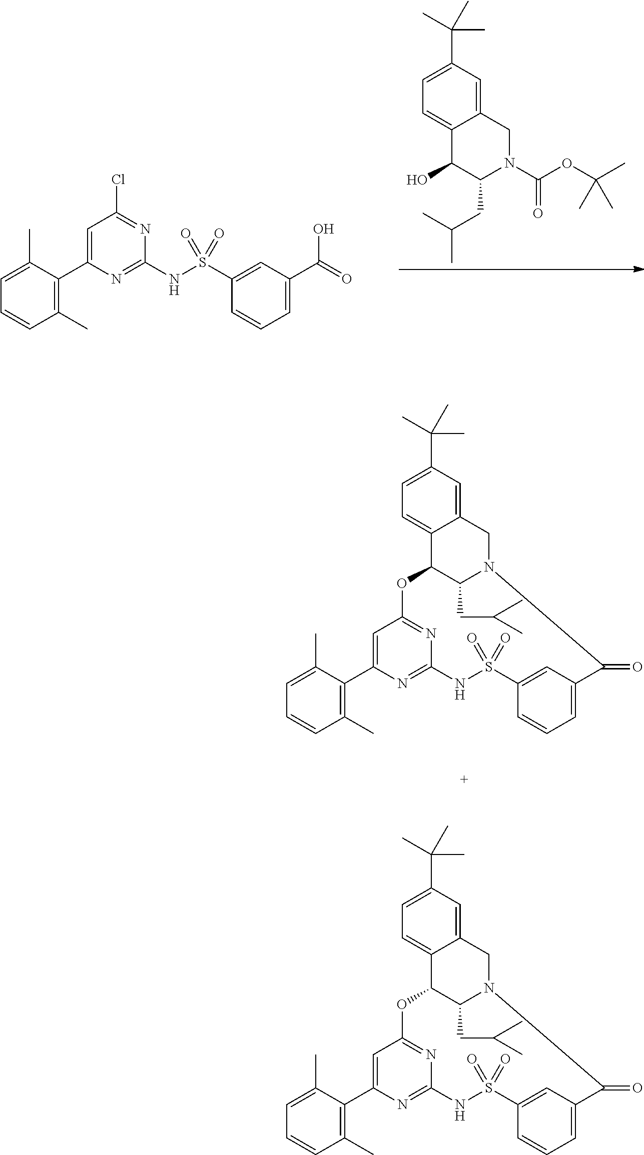 [SMILES: Cc1cccc(C)c1-c1cc(Cl)nc(NS(=O)(=O)c2cccc(C(=O)O)c2)n1 ; CC(C)C[C@@H]1[C@@H](O)c2ccc(C(C)(C)C)cc2CN1C(=O)OC(C)(C)C ; Cc1cccc(C)c1-c1cc2nc(n1)NS(=O)(=O)c1cccc(c1)C(=O)N1Cc3cc(C(C)(C)C)ccc3[C@H](O2)[C@H]1CC(C)C ; Cc1cccc(C)c1-c1cc2nc(n1)NS(=O)(=O)c1cccc(c1)C(=O)N1Cc3cc(C(C)(C)C)ccc3[C@@H](O2)[C@H]1CC(C)C]

In a 20-mL vial, tert-butyl (3R,4S)-7-tert-butyl-4-hydroxy-3-isobutyl-3,4-dihydro-1H-isoquinoline-2-carboxylate (260.7 mg, 0.7211 mmol) was dissolved in dioxane (3.0 mL), to which a dioxane solution of HCl (3.0 mL of 4.0 M, 12.00 mmol) was added. This mixture was stirred at room temperature for 4.5 h. This mixture was then evaporated to dryness in vacuo to give 224.6 mg (>100% yield) of a yellow solid. In a 20-mL vial, the product was mixed with THE (3.0 mL), to which NaOtBu (512.2 mg, 5.330 mmol) as added. This mixture was stirred at room temperature for 10 min, after which it was cooled to 0° C. Then, 3-[[4-chloro-6-(2,6-dimethylphenyl)pyrimidin-2-yl]sulfamoyl]benzoic acid (349.1 mg, 0.8354 mmol) was added, and this mixture was stirred at 0° C. for 1 h, and at room temperature for 1 h. In a separate 20-mL vial, a solution of HATU (845.2 mg, 2.223 mmol) in DMF (6.0 mL) was prepared. The above-prepared reaction mixture was added dropwise onto this HATU solution, and the resulting mixture was stirred at room temperature for 15 min. This mixture was then quenched with 1 N HCl solution (30 mL) and diluted with ethyl acetate (120 mL). The layers were separated, and the organic layer was washed with 1 N HCl solution (40 mL), water (40 mL) and saturated aqueous sodium chloride solution (40 mL), then dried over sodium sulfate, filtered, and evaporated in vacuo to give a yellow solid. Purification by silica gel chromatography (24 g of silica column) using a gradient eluent of 1 to 70% ethyl acetate in hexanes gave 2 batches of product (80 mg of 70% pure material and 125 mg of 60% pure material). These were dissolved separately in warm DMSO (2 mL each) and purified by reverse phase preparative chromatography using a C₁₈ column and a gradient eluent of 1 to 99% acetonitrile in water containing 5 mM hydrochloric acid to give: major product, (16S,24R)-20-tert-butyl-12-(2,6-dimethylphenyl)-24-(2-methylpropyl)-15-oxa-8λ⁶-thia-1,9,11,25-tetraazapentacyclo[14.7.1.13,7.110,14.017,22]hexacosa-3,5,7(26),10,12,14(25),17,19,21-nonaene-2,8,8-trione (122.5 mg, 27%)¹H NMR (400 MHz, DMSO-d₆) δ 12.17-11.55 (bs, 1H), 8.44 (t, J=1.8 Hz, 1H), 7.89 (d, J=7.7 Hz, 1H), 7.69 (d, J=7.6 Hz, 1H), 7.64 (t, J=7.6 Hz, 1H), 7.58 (d, J=8.1 Hz, 1H), 7.45 (d, J=2.0 Hz, 1H), 7.44-7.39 (m, 1H), 7.41 (s, 1H), 7.25 (t, J=7.6 Hz, 1H), 7.13 (d, J=7.6 Hz, 2H), 5.53 (s, 1H), 5.48 (d, J=18.1 Hz, 1H), 4.25 (d, J=18.1 Hz, 1H), 3.48-3.30 (m, 1H, hidden under water peak), 2.08-1.72 (bs, 6H), 1.44-1.19 (m, 3H), 1.33 (s, 9H), 0.56 (d, J=6.3 Hz, 3H), 0.44 (d, J=6.3 Hz, 3H) ESI-MS m/z calc. 624.27704, found 625.5 (M+1)⁺; Retention time: 2.13 minutes (LC method A); and minor product, (16R,24R)-20-tert-butyl-12-(2,6-dimethylphenyl)-24-(2-methylpropyl)-15-oxa-8λ⁶-thia-1,9,11,25-tetraazapentacyclo[14.7.1.13,7.110,14.017,22]hexacosa-3,5,7(26),10,12,14(25),17,19,21-nonaene-2,8,8-trione (5.9 mg, 1%)¹H NMR (400 MHz, DMSO-d₆) δ 12.22-11.61 (bs, 1H), 8.45 (s, 1H), 7.89 (d, J=7.7 Hz, 1H), 7.73-7.58 (m, 3H), 7.50 (dd, J=8.2, 2.1 Hz, 1H), 7.48 (s, 1H), 7.25 (t, J=7.6 Hz, 1H), 7.13 (d, J=7.6 Hz, 2H), 5.52 (s, 1H), 5.40 (d, J=18.1 Hz, 1H), 4.22 (d, J=18.1 Hz, 1H), 3.48-3.30 (m, 1H, hidden under water peak), 2.11-1.81 (bs, 6H), 1.43-1.15 (m, 3H), 1.30 (s, 9H), 0.55 (d, J=6.2 Hz, 3H), 0.44 (d, J=6.3 Hz, 3H) ESI-MS m/z calc. 624.27704, found 625.5 (M+1)⁺; Retention time: 2.1 minutes (LC method A); and side product, (16S,24R)-20-tert-butyl-4-(2,6-dimethylphenyl)-24-(2-methylpropyl)-15-oxa-8λ⁶-thia-1,5,7,26-tetraazapentacyclo[14.7.1.12,6.19,13.017,22]hexacosa-2(26),3,5,9(25),10,12,17,19,21-nonaene-8,8,14-trione (16.3 mg, 4%) ESI-MS m/z calc. 624.27704, found 625.5 (M+1)⁺; Retention time: 2.22 minutes (LC method A).

### Example 88: Preparation of Compound 186 and Compound 187

### Step 1: 2-[2-Bromo-5-(trifluoromethyl)phenyl]-1,3-dioxolane

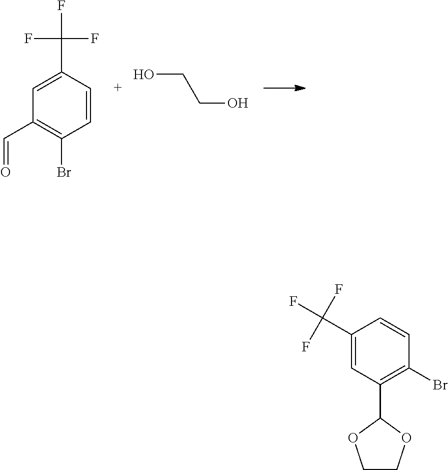

2-Bromo-5-(trifluoromethyl)benzaldehyde (25 g, 93.868 mmol) was dissolved in benzene (450 mL). ethylene glycol (5.8425 g, 5.3 mL, 93.190 mmol) was added, followed by pTSA hydrate (893 mg, 4.6242 mmol). The mixture was heated in a round bottom flask equipped with a Dean-Stark set up and stirred under nitrogen balloon in a 100° C. oil bath for 16 h. The reaction was cooled to rt and diluted with water. The layers were separated, and the organic layer was washed with saturated aqueous sodium bicarbonate, dried over sodium sulfate, filtered and concentrated. The residue was purified by silica gel chromatography using 0 to 40% EtOAc in Hexanes to afford 2-[2-Bromo-5-(trifluoromethyl)phenyl]-1,3-dioxolane as a colorless oil. ESI-MS m/z calc. 295.966, found 295.2 (M−1)−; Retention time: 4.08 minutes, LC method T.

### Step 2: tert-Butyl N-[(1R)-1-[2-(1,3-dioxolan-2-yl)-4-(trifluoromethyl)benzoyl]-3-methyl-butyl]carbamate

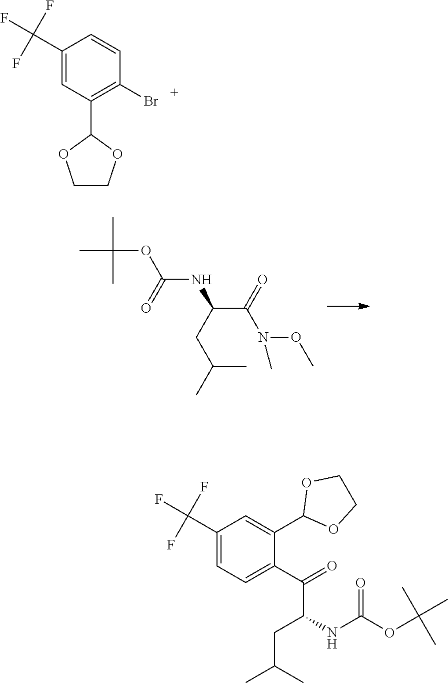

2-[2-Bromo-5-(trifluoromethyl)phenyl]-1,3-dioxolane (14.62 g, 46.753 mmol) was dissolved in toluene (55 mL), cooled in ice water bath under nitrogen balloon and stirred 15 min. nBuLi (19 mL of 2.5 M, 47.500 mmol) was added quickly dropwise. The mixture became difficult to stir. More toluene (30 ml total) was added along the wall of the reaction flask. The mixture still needed some manual swirling. Mixture was kept at 0° C. for 45 min. A tert-butyl N-[(1R)-1-[methoxy(methyl)carbamoyl]-3-methyl-butyl]carbamate (5.4 g, 18.698 mmol) solution in toluene (20 ml plus 5 ml rinse) was added by syringe. The resulted mixture was allowed to stir with ice bath removed for 45 min. ammonium chloride (20 ml. saturated aqueous) was added. The mixture was extracted with EtOAc (50 ml×2). The combined organics were washed with brine, dried over anhydrous sodium sulfate, filtered and concentrated. The residue was purified by silica gel chromatography, using 0-40% EtOAc in Hexanes (Rt: 0.6 (1/3 EtOAc/Hexanes) to give (5.8 g, 68%) as a colorless oil. ESI-MS m/z calc. 431.192, found 432.7 (M+1)⁺; Retention time: 4.29 minutes, LC method T.

### Step 3: tert-Butyl N-[(1R)-1-[[2-(1,3-dioxolan-2-yl)-4-(trifluoromethyl)phenyl]-hydroxy-methyl]-3-methyl-butyl]carbamate

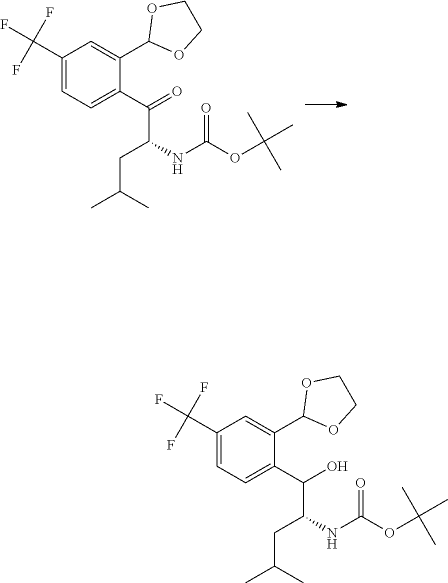 [SMILES: CC(C)C[C@@H](NC(=O)OC(C)(C)C)C(=O)c1ccc(C(F)(F)F)cc1C1OCCO1 ; CC(C)C[C@@H](NC(=O)OC(C)(C)C)C(O)c1ccc(C(F)(F)F)cc1C1OCCO1]

tert-Butyl N-[(1R)-1-[2-(1,3-dioxolan-2-yl)-4-(trifluoromethyl)benzoyl]-3-methyl-butyl]carbamate (4 g, 8.8076 mmol) was dissolved in EtOH (50 mL) and cooled in ice water bath. Sodium borohydride (740 mg, 19.169 mmol) was added in small portions. The mixture was stirred for 10 min. MeOH (5 mL) was then added. The reaction was continued for 30 min. ammonium chloride (20 ml, saturated aqueous) was added. The mixture was concentrated to remove most volatiles and the residue was partitioned between water (40 mL) and DCM (50 mL). Layers were separated and the DCM solution was dried over anhydrous sodium sulfate, filtered and concentrated to afford tert-butyl N-[(1R)-1-[[2-(1,3-dioxolan-2-yl)-4-(trifluoromethyl)phenyl]-hydroxy-methyl]-3-methyl-butyl]carbamate (4 g, 100%) as a mixture of isomers. ESI-MS m/z calc. 433.2076, found 434.7 (M+1)⁺; Retention time: 4.1 minutes, LC method T.

### Step 4: tert-Butyl (3R)-4-hydroxy-3-isobutyl-7-(trifluoromethyl)-3,4-dihydro-1H-isoquinoline-2-carboxylate

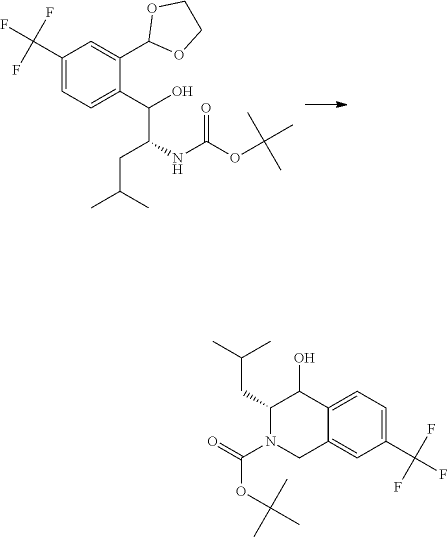

tert-Butyl N-[(1R)-1-[[2-(1,3-dioxolan-2-yl)-4-(trifluoromethyl)phenyl]-hydroxy-methyl]-3-methyl-butyl]carbamate (4 g, 9.1357 mmol) was dissolved in HCl (30 mL of 4 M, 120.00 mmol) and stirred at RT for 4 h. It was then concentrated. The crude residue (imine) was taken into EtOH (20 mL) and cooled in ice water bath. Sodium borohydride (520 mg, 0.5503 mL, 13.745 mmol) was added in small portions. The mixture was stirred at RT for 1 hour and then ammonium chloride (20 ml, saturated aqueous) was added. The mixture was concentrated under vacuum to remove most EtOH. The residue was partitioned between water (˜30 mL) and DCM (40 mL). Layers were separated and the aqueous layer was further extracted with DCM (20 mL). The combined DCM solution was cooled in ice water bath. TEA (1.4520 g, 2 mL, 14.349 mmol) was added, followed by Boc anhydride (3 g, 3.1579 mL, 13.746 mmol). The mixture was then stirred at RT under nitrogen for 15 h. Water (40 mL) was added. The separated DCM layer was further washed with HCl (20 ml, 1 N aqueous), saturated sodium bicarbonate and brine. It was then dried over anhydrous sodium sulfate, filtered and concentrated. The residue was purified by silica gel chromatography (80 g column), using 5-40% EtOAc in Hexanes over 90 min to give tert-butyl (3R)-4-hydroxy-3-isobutyl-7-(trifluoromethyl)-3,4-dihydro-1H-isoquinoline-2-carboxylate (1.62 g, 47%) ESI-MS m/z calc. 373.1865, found 374.4 (M+1)⁺; Retention time: 4.22 minutes. ¹H NMR (250 MHz, DMSO-d₆) δ 7.72-7.45 (m, 3H), 5.45 (d, J=5.3 Hz, 1H), 4.93 (t, J=15.6 Hz, 1H), 4.58-4.31 (m, 2H), 4.28-4.01 (m, 1H), 1.44 (s, 9H), 1.16-0.63 (m, 9H). LC method W.

### Step 5: (16S,24R)-12-(2,6-Dimethylphenyl)-24-(2-methylpropyl)-20-(trifluoromethyl)-15-oxa-8λ⁶-thia-1,9,11,25-tetraazapentacyclo[14.7.1.13,7.110,14.017,22]hexacosa-3,5,7(26),10,12,14(25),17,19,21-nonaene-2,8,8-trione (Compound 186) and (16R,24R)-12-(2,6-dimethylphenyl)-24-(2-methylpropyl)-20-(trifluoromethyl)-15-oxa-8λ⁶-thia-1,9,11,25-tetraazapentacyclo[14.7.1.13,7.110,14.017,22]hexacosa-3,5,7(26),10,12,14(25),17,19,21-nonaene-2,8,8-trione (Compound 187)

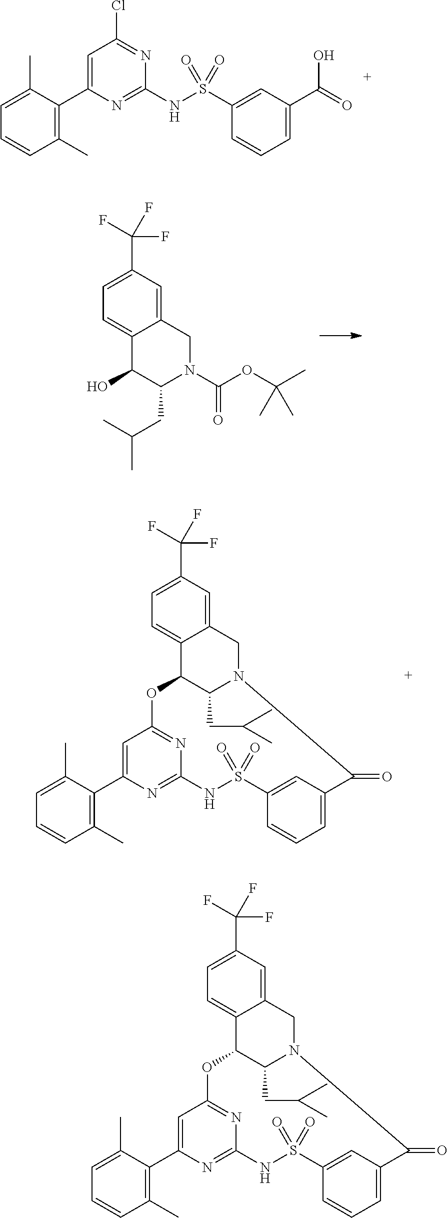 [SMILES: Cc1cccc(C)c1-c1cc(Cl)nc(NS(=O)(=O)c2cccc(C(=O)O)c2)n1 ; CC(C)C[C@@H]1[C@@H](O)c2ccc(C(F)(F)F)cc2CN1C(=O)OC(C)(C)C ; Cc1cccc(C)c1-c1cc2nc(n1)NS(=O)(=O)c1cccc(c1)C(=O)N1Cc3cc(C(F)(F)F)ccc3[C@H](O2)[C@H]1CC(C)C ; Cc1cccc(C)c1-c1cc2nc(n1)NS(=O)(=O)c1cccc(c1)C(=O)N1Cc3cc(C(F)(F)F)ccc3[C@@H](O2)[C@H]1CC(C)C]

In a 100-mL round-bottomed flask, tert-butyl (3R,4S)-4-hydroxy-3-isobutyl-7-(trifluoromethyl)-3,4-dihydro-1H-isoquinoline-2-carboxylate (801.2 mg, 2.146 mmol) was dissolved in dioxane (10 mL), to which a dioxane solution of HCl (10 mL of 4.0 M, 40.00 mmol) was added. This mixture was stirred at room temperature for 6.5 h. This mixture was then evaporated to dryness in vacuo to give 1.4 g (>100% yield) of a light-yellow solid. In a 100-mL round-bottomed flask, the product 1 was dissolved in THE (10 mL), to which NaOtBu (1.5128 g, 15.74 mmol) as added. This mixture was stirred at room temperature for 10 min, after which it was cooled to 0° C. Then, 3-[[4-chloro-6-(2,6-dimethylphenyl)pyrimidin-2-yl]sulfamoyl]benzoic acid (965.4 mg, 2.310 mmol) was added, and this mixture was stirred at 0° C. for 2 h. In a separate 100-mL round-bottomed flask, a solution of HATU (2.5088 g, 6.598 mmol) in DMF (20 mL) was prepared. The above-prepared reaction mixture was added dropwise onto this HATU solution, and the resulting mixture was stirred at room temperature for 15 min. This mixture was then quenched with 1 N HCl solution (60 mL) and diluted with ethyl acetate (250 mL). The layers were separated, and the organic layer was washed with 1 N HCl solution (100 mL), water (100 mL) and saturated aqueous sodium chloride solution (100 mL), then dried over sodium sulfate, filtered, and evaporated in vacuo to give a yellow slurry. Purification by silica gel chromatography (solid loading with 10 g Celite; 40 g of silica column) using a gradient eluent of 1 to 70% ethyl acetate in hexanes gave 3 batches of product (a minor diastereomer batch, a mixed batch, and a mostly pure major diastereomer batch). These were dissolved separately in 1:1 DMSO:MeOH and purified by reverse phase preparative chromatography using a C₁₈ column and a gradient eluent of 1 to 99% acetonitrile in water containing 5 mM hydrochloric acid to give some mixed batches, as well as pure products: Major product, (16S,24R)-12-(2,6-dimethylphenyl)-24-(2-methylpropyl)-20-(trifluoromethyl)-15-oxa-8λ⁶-thia-1,9,11,25-tetraazapentacyclo[14.7.1.13,7.110,14.017,22]hexacosa-3,5,7(26),10,12,14(25),17,19,21-nonaene-2,8,8-trione (70.6 mg, 5%)¹H NMR (400 MHz, DMSO-d₆) δ 12.40-11.38 (bs, 1H), 8.45 (t, J=1.8 Hz, 1H), 7.96-7.88 (m, 3H), 7.78-7.70 (m, 2H), 7.66 (t, J=7.7 Hz, 1H), 7.49 (s, 1H), 7.25 (t, J=7.6 Hz, 1H), 7.13 (d, J=7.7 Hz, 2H), 5.63 (s, 1H), 5.60 (d, J=18.6 Hz, 1H), 4.35 (d, J=18.6 Hz, 1H), 3.41 (t, J=7.2 Hz, 1H), 2.11-1.79 (bs, 6H), 1.45-1.28 (m, 2H), 1.28-1.17 (m, 1H), 0.56 (d, J=6.2 Hz, 3H), 0.41 (d, J=6.2 Hz, 3H) ESI-MS m/z calc. 636.2018, found 637.3 (M+1)⁺; Retention time: 1.96 minutes (LC method A); and a minor product, (16R,24R)-12-(2,6-dimethylphenyl)-24-(2-methylpropyl)-20-(trifluoromethyl)-15-oxa-8λ⁶-thia-1,9,11,25-tetraazapentacyclo[14.7.1.13,7.110,14.017,22]hexacosa-3,5,7(26),10,12,14(25),17,19,21-nonaene-2,8,8-trione (34.8 mg, 3%)¹H NMR (400 MHz, DMSO-d₆) δ 13.75-11.52 (broad d, 1H), 8.45 (s, 1H), 8.03-7.91 (m, 1H), 7.84 (s, 1H), 7.81-7.56 (m, 4H), 7.35-7.21 (m, 2H), 7.21-7.08 (m, 2H), 6.59 (s, 1H), 5.70 (d, J=17.2 Hz, 1H), 4.62 (d, J=17.1 Hz, 1H), 3.85 (td, J 10.6, 5.8 Hz, 1H), 2.29-1.85 (bs, 6H), 1.42-1.29 (m, 1H), 0.59 (d, J=6.5 Hz, 3H), 0.41 (d, J=6.4 Hz, 3H), 0.41-0.33 (m, 1H), 0.19-0.01 (m, 1H) ESI-MS m/z calc. 636.2018, found 637.3 (M+1)⁺; Retention time: 2.03 minutes, LC method A.

### Example 89: Preparation of Compound 188 and Compound 189

### Step 1: (2R)-2-(tert-Butoxycarbonylamino)-4,4-dimethyl-pentanoic acid

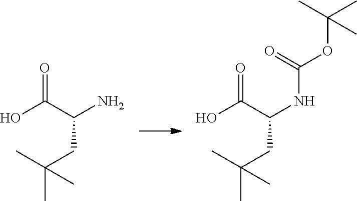

To a solution of (2R)-2-amino-4,4-dimethyl-pentanoic acid (10 g, 68.871 mmol) in water (70 mL) was added NaOH (3.0301 g, 75.758 mmol) followed by a solution of tert-butoxycarbonyl tert-butyl carbonate (15.8 g, 16.632 mL, 72.395 mmol) in THE (70 mL). The cloudy mixture, which gradually became clear and then cloudy again, was stirred at room temperature overnight. Most of the THF was evaporated and the residue was acidified with 1N HCl (76 mL) and extracted with DCM (3×100 mL. The combined organic phases were dried, filtered and evaporated to give (2R)-2-(tert-butoxycarbonylamino)-4,4-dimethyl-pentanoic acid (16.8 g, 99%) as a white solid. ¹H NMR (300 MHz, CDCl₃): δ 12.40 (s, 1H), 7.07 (d, J=8.5 Hz, 1H), 3.98-3.90 (m, 1H), 1.54 (d, J=6.7 Hz, 2H), 1.37 (s, 9H), 0.88 (s, 9H). ESI-MS m/z calc. 245.1627, found 268.2 (M+Na)⁺; Retention time: 1.89 minutes, LC method K.

### Step 2: tert-Butyl N-[(1R)-1-[methoxy(methyl)carbamoyl]-3,3-dimethyl-butyl]carbamate

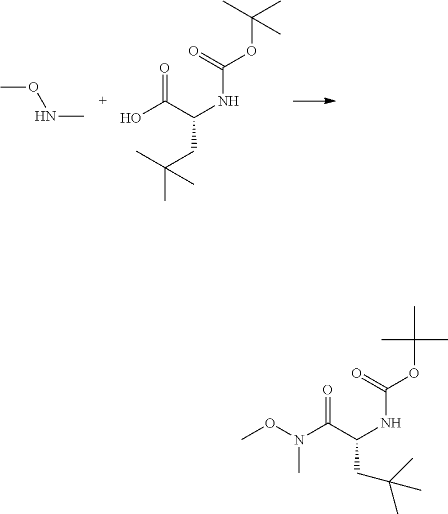

A solution of (2R)-2-(tert-butoxycarbonylamino)-4,4-dimethyl-pentanoic acid (16.8 g, 68.483 mmol) and N-methoxymethanamine (hydrochloride salt) (8 g, 82.014 mmol) in DMF (120 mL) was cooled in an ice bath and treated with HATU (28.6 g, 75.218 mmol). After about 10-15 minutes, DIPEA (19.515 g, 26.3 mL, 150.99 mmol) was added, the ice bath was removed and the reaction was left to stir at room temperature overnight. The reaction was cooled with ice-water bath. Water (350 mL) was added. The mixture was vigorously stirred for 30 minutes and filtered. The solid was dissolved in EtOAc (300 mL). The solution was washed with brine (40 mL). The organic layer was dried over sodium sulfate, filtered and concentrated under reduced pressure to afford crude tert-butyl N-[(1R)-1-[methoxy(methyl)carbamoyl]-3,3-dimethyl-butyl]carbamate (15.8 g, 80%) as a white solid, which was used in the following step without further purification. ¹H NMR (300 MHz, CDCl₃): δ 5.06-4.94 (m, 1H), 4.84-4.70 (m, 1H), 3.79 (s, 3H), 3.19 (s, 3H), 1.54-1.33 (m, 11H), 0.97 (s, 9H). (M+Na)⁺=311.2, retention time: 1.99 minutes, LC method K.

### Step 3: tert-Butyl N-[(1R)-1-[4-tert-butyl-2-(1,3-dioxolan-2-yl)benzoyl]-3,3-dimethyl-butyl]carbamate

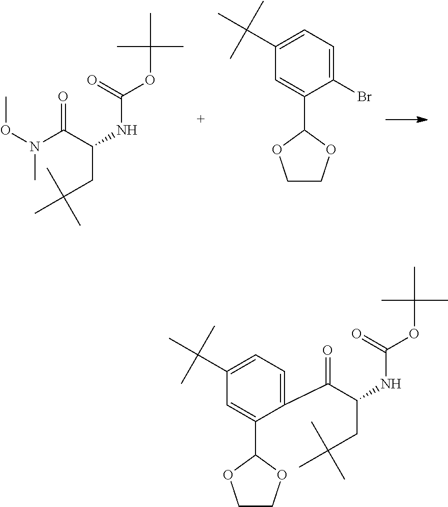 [SMILES: CON(C)C(=O)[C@@H](CC(C)(C)C)NC(=O)OC(C)(C)C ; CC(C)(C)c1ccc(Br)c(C2OCCO2)c1 ; CC(C)(C)C[C@@H](NC(=O)OC(C)(C)C)C(=O)c1ccc(C(C)(C)C)cc1C1OCCO1]

n-BuLi (7.2 mL of 2.5 M, 18.00 mmol) (in hexanes) was added to a toluene (18 mL) solution of 2-(2-bromo-5-tert-butyl-phenyl)-1,3-dioxolane (5.14 g, 18.02 mmol) at 0° C. and it was stirred for 30 minutes. A solution of tert-butyl N-[(1R)-1-[methoxy(methyl)carbamoyl]-3,3-dimethyl-butyl]carbamate (2.07 g, 7.178 mmol) in anhydrous toluene (9 mL) was added to the reaction mixture at −10 to 0° C. The reaction was stirred at this temperature for 1 hour, then it was quenched with saturated ammonium chloride aqueous solution (50 mL) at 0° C. Two layers were separated, and the aqueous layer was extracted with diethyl ether (2×50 mL). The combined organic layers were washed with brine (50 mL), dried over anhydrous sodium sulfate and concentrated under vacuum. The residue was purified by silica gel chromatography using 0 to 30% diethyl ether in hexanes (120 g column) to give tert-butyl N-[(1R)-1-[4-tert-butyl-2-(1,3-dioxolan-2-yl)benzoyl]-3,3-dimethyl-butyl]carbamate (1.32 g, 42%) ESI-MS m/z calc. 433.28284, found 378.4 (M-tBu)−; Retention time: 2.34 minutes, LC method A.

### Step 4: tert-Butyl N-[(1R)-1-[(S)-[4-tert-butyl-2-(1,3-dioxolan-2-yl)phenyl]-hydroxy-methyl]-3,3-dimethyl-butyl]carbamate

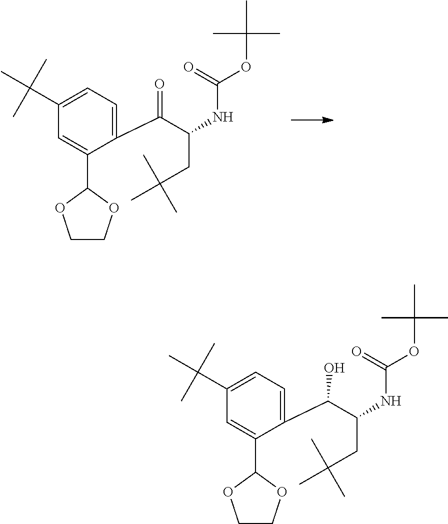

A EtOH (12 mL) solution of tert-butyl N-[(1R)-1-[4-tert-butyl-2-(1,3-dioxolan-2-yl)benzoyl]-3,3-dimethyl-butyl]carbamate (1.32 g, 3.044 mmol) was treated with sodium borohydride (117.2 mg, 3.098 mmol) at room temperature and stirred for 50 minutes. The reaction was carefully quenched with saturated aqueous ammonium chloride (NOTE: gas evolution, 15 mL) and diluted with ethyl acetate (30 mL). The organic layer was separated, dried over anhydrous sodium sulfate, filtered, and concentrated in vacuo to give tert-butyl N-[(1R)-1-[(S)-[4-tert-butyl-2-(1,3-dioxolan-2-yl)phenyl]-hydroxy-methyl]-3,3-dimethyl-butyl]carbamate (1.27 g, 96%) ESI-MS m/z calc. 435.29846, found 374.4 (M+1)⁺; Retention time: 2.15 minutes, LC method A.

### Step 5: (3R,4S)-7-tert-Butyl-3-(2,2-dimethylpropyl)-1,2,3,4-tetrahydroisoquinolin-4-ol

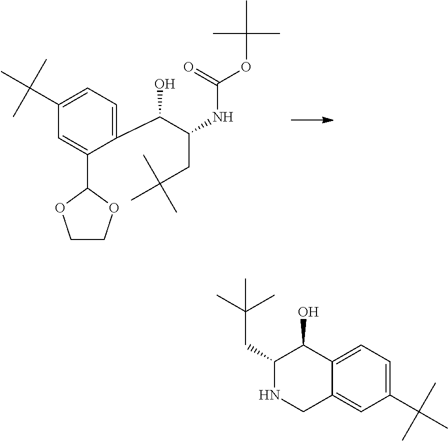

tert-Butyl N-[(1R)-1-[[4-tert-butyl-2-(1,3-dioxolan-2-yl)phenyl]-hydroxy-methyl]-3,3-dimethyl-butyl]carbamate (1.27 g, 2.916 mmol) was treated with hydrogen chloride (10 mL of 4 M, 40.00 mmol) at room temperature, stirred for 50 minutes, and then concentrated in vacuo. The crude residue was dissolved in EtOH (15 mL) and cooled to 0° C. and treated with sodium borohydride (165.8 mg, 4.295 mmol) portionwise. The reaction was stirred for 2 hours and concentrated in vacuo. The residue was diluted with ethyl acetate (50 mL) and carefully quenched with a saturated solution of aqueous ammonium chloride (30 mL). The aqueous layer was extracted with EtOAc (20 mL) and the combined organic layers were dried over sodium sulfate and concentrated in vacuo. The crude residue was dissolved in THE (15 mL) and treated with Boc anhydride (994.4 mg, 4.556 mmol) followed by triethylamine (900 μL, 6.457 mmol) and the reaction mixture was stirred for 16 hours and then diluted with a saturated solution of aqueous ammonium chloride (40 mL) followed by ethyl acetate (40 mL). The organic layer was separated, dried over anhydrous sodium sulfate, filtered, and concentrated in vacuo. The residue was loaded on to silica gel (80 g) and purified by flash column chromatography using 0-5% MeOH/DCM as an eluent over 35 min. The appropriate fractions were collected to give tert-butyl (3R,4S)-7-tert-butyl-3-(2,2-dimethylpropyl)-4-hydroxy-3,4-dihydro-1H-isoquinoline-2-carboxylate ESI-MS m/z calc. 375.27734, found 302.4 (M-OtBu)+; Retention time: 0.88 minutes (LC method D). The product from above was treated with HCl (16 mL of 4 M, 64.00 mmol) and stirred at room temperature for 1 hour and then concentrated in vacuo to give (3R,4S)-7-tert-butyl-3-(2,2-dimethylpropyl)-1,2,3,4-tetrahydroisoquinolin-4-ol (hydrochloride salt) ESI-MS m/z calc. 275.2249, found 276.3 (M+1)⁺; Retention time: 0.5 minutes (LC method D).

### Step 6: (16S,24R)-20-tert-Butyl-12-(2,6-dimethylphenyl)-24-(2,2-dimethylpropyl)-15-oxa-8λ⁶-thia-1,9,11,25-tetraazapentacyclo[14.7.1.13,7.110,14.017,22]hexacosa-3(26),4,6,10(25),11,13,17,19,21-nonaene-2,8,8-trione (Compound 188) and (16R,24R)-20-tert-butyl-12-(2,6-dimethylphenyl)-24-(2,2-dimethylpropyl)-15-oxa-8λ⁶-thia-1,9,11,25-tetraazapentacyclo[14.7.1.13,7.110,14.017,22]hexacosa-3(26),4,6,10(25),11,13,17,19,21-nonaene-2,8,8-trione (Compound 189)

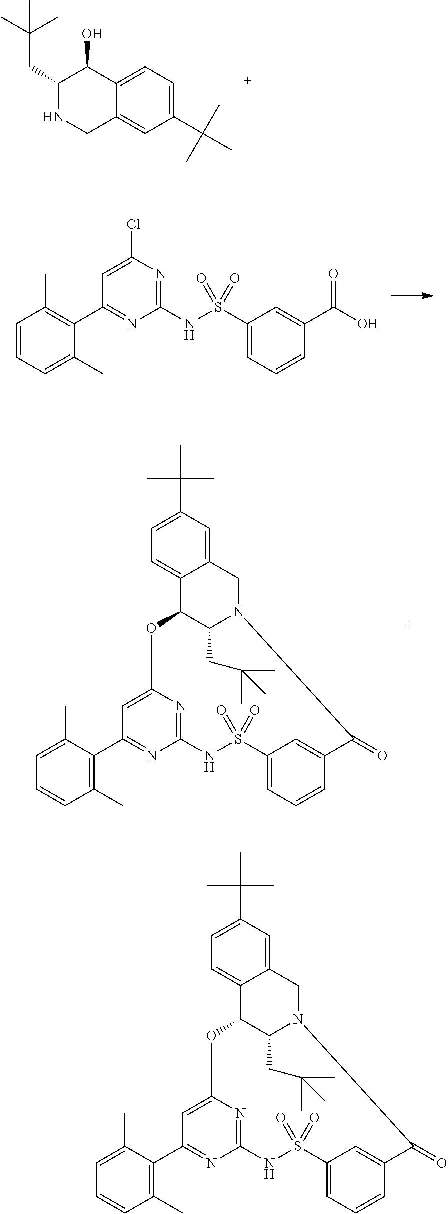 [SMILES: CC(C)(C)C[C@H]1NCc2cc(C(C)(C)C)ccc2[C@@H]1O ; Cc1cccc(C)c1-c1cc(Cl)nc(NS(=O)(=O)c2cccc(C(=O)O)c2)n1 ; Cc1cccc(C)c1-c1cc2nc(n1)NS(=O)(=O)c1cccc(c1)C(=O)N1Cc3cc(C(C)(C)C)ccc3[C@H](O2)[C@H]1CC(C)(C)C ; Cc1cccc(C)c1-c1cc2nc(n1)NS(=O)(=O)c1cccc(c1)C(=O)N1Cc3cc(C(C)(C)C)ccc3[C@@H](O2)[C@H]1CC(C)(C)C]

In a 20-mL vial, (3R,4S)-7-tert-butyl-3-(2,2-dimethylpropyl)-1,2,3,4-tetrahydroisoquinolin-4-ol (307 mg, 1.115 mmol) was mixed with THE (4.0 mL), to which sodium tert-butoxide (746.7 mg, 7.770 mmol) was added. This mixture was stirred at room temperature for 10 min, after which it was cooled to 0° C. Then, 3-[[4-chloro-6-(2,6-dimethylphenyl)pyrimidin-2-yl]sulfamoyl]benzoic acid (540.4 mg, 1.293 mmol) was added, and this mixture was stirred at 0° C. for 1 h, and at room temperature for 1 h. The reaction mixture was then transferred dropwise using a pipette into a DMF (9 mL) solution of HATU (1.2713 g, 3.344 mmol) at room temperature and the reaction was stirred for 15 minutes. This reaction mixture was then quenched with 1 N HCl solution (30 mL) and diluted with ethyl acetate (120 mL). The layers were separated, and the organic layer was washed with 1 N HCl solution (40 mL), water (40 mL) and saturated aqueous sodium chloride solution (40 mL), then dried over sodium sulfate, filtered, and evaporated in vacuo to give a yellow solid. Purification by silica gel chromatography (24 g of silica column) using a gradient eluent of 1 to 70% ethyl acetate in hexanes gave 2 batches of semi pure products. These were dissolved separately in hot DMSO (2 mL each) and purified by reverse phase preparative chromatography using a C₁₈ column and a gradient eluent of 1 to 99% acetonitrile in water containing 5 mM hydrochloric acid to give (16S,24R)-20-tert-butyl-12-(2,6-dimethylphenyl)-24-(2,2-dimethylpropyl)-15-oxa-8λ⁶-thia-1,9,11,25-tetraazapentacyclo[14.7.1.13,7.110,14.017,22]hexacosa-3(26),4,6,10(25),11,13,17,19,21-nonaene-2,8,8-trione (19.8 mg, 3%) ESI-MS m/z calc. 638.29266, found 639.5 (M+1)⁺; Retention time: 2.29 minutes (major diastereomer) and (16R,24R)-20-tert-butyl-12-(2,6-dimethylphenyl)-24-(2,2-dimethylpropyl)-15-oxa-8λ⁶-thia-1,9,11,25-tetraazapentacyclo[14.7.1.13,7.110,14.017,22]hexacosa-3(26),4,6,10(25),11,13,17,19,21-nonaene-2,8,8-trione (2.5 mg, 0%) ESI-MS m/z calc. 638.29266, found 639.5 (M+1)⁺; Retention time: 2.37 minutes (minor diastereomer), LC method A.

### Example 90: Preparation of Compound 190

### Step 1: tert-Butyl 2-[(3-methyl-1-phenyl-butyl)amino]acetate

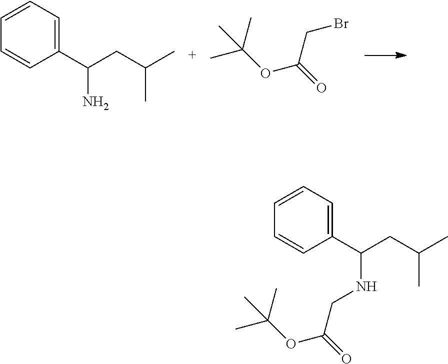

To a solution of 3-methyl-1-phenyl-butan-1-amine (hydrochloride salt) (5 g, 25.035 mmol) in DMF (100 mL) at 0° C. was added potassium carbonate (7.3 g, 52.820 mmol), followed by tert-butyl 2-bromoacetate (5.03 g, 25.788 mmol) dropwise. The mixture was stirred at 0° C. for 4 hours and allowed to slowly warm to rt and stirred overnight. Ice-water (200 g) was added. The mixture was extracted with DCM (200 mL) and dried with sodium sulfate. Flash chromatography (120 g silica gel, DCM/MeOH 0-5%) afforded tert-butyl 2-[(3-methyl-1-phenyl-butyl)amino]acetate (5.78 g, 83%) as light yellow oil. ESI-MS m/z calc. 277.2042, found 278.3 (M+1)⁺; Retention time: 1.64 minutes. ¹H NMR (300 MHz, CDCl₃) δ 7.44-7.12 (m, 5H), 3.72-3.56 (m, 1H), 3.20-2.99 (m, 2H), 1.85 (br. s., 1H), 1.66-1.45 (m, 3H), 1.43 (s, 9H), 0.89 (dd, J=8.5, 6.5 Hz, 6H), LC method K.

### Step 2: tert-Butyl 2-[(3-methyl-1-phenyl-butyl)-(2,2,2-trifluoroacetyl)amino]acetate

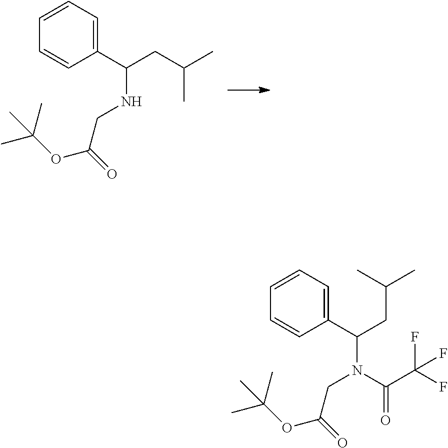 [SMILES: CC(C)CC(NCC(=O)OC(C)(C)C)c1ccccc1 ; CC(C)CC(c1ccccc1)N(CC(=O)OC(C)(C)C)C(=O)C(F)(F)F]

To a solution of tert-butyl 2-[(3-methyl-1-phenyl-butyl)amino]acetate (3.02 g, 10.887 mmol) in DCM (20 mL) at 0° C. was added TEA (5.0094 g, 6.9 mL, 49.505 mmol), followed by a solution of TFAA (3.48 g, 2.3031 mL, 16.569 mmol) in DCM (10 mL) dropwise. The mixture was stirred at rt for 1.5 h. HPLC (1) showed the reaction was complete. The mixture was cooled with ice-water bath. 60 mL of 5% aqueous sodium bicarbonate was added slowly. The mixture was extracted with DCM (100 mL) and dried with sodium sulfate. Flash chromatography (24 g silica gel, heptanes/EtOAc 0-35%) afforded tert-butyl 2-[(3-methyl-1-phenyl-butyl)-(2,2,2-trifluoroacetyl)amino]acetate (3.91 g, 96%) as light yellow oil. ESI-MS m/z calc. 373.1865, found 396.2 (M+Na)⁺; Retention time: 2.4 minutes ¹H NMR (300 MHz, CDCl₃) δ 7.44-7.28 (m, 5H), 5.89 (t, J=7.8 Hz, 0.5H), 5.22 (dd, J=10.4, 4.6 Hz, 0.5H), 3.83 (dd, J=4.6, 1.6 Hz, 1H), 3.76 (s, 1H), 2.13 (ddd, J=13.3, 10.1, 3.7 Hz, 1H), 1.89-1.57 (m, 2H), 1.32 (s, 5H), 1.29 (s, 4H), 1.00-0.87 (m, 6H). ¹⁹19F NMR (282 MHz, CDCl₃) δ −67.47 (s, 1.6F), −69.20 (s, 1.4F). LC method K.

### Step 3: 2-[(3-Methyl-1-phenyl-butyl)-(2,2,2-trifluoroacetyl)amino]acetic acid

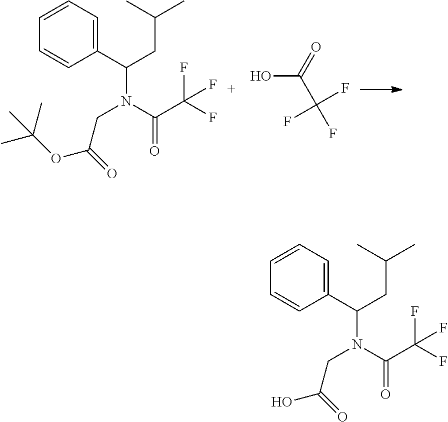

To a solution of tert-butyl 2-[(3-methyl-1-phenyl-butyl)-(2,2,2-trifluoroacetyl)amino]acetate (3.91 g, 10.471 mmol) in DCM (50 mL) at 0° C. was added TFA (37.000 g, 25 mL, 324.50 mmol). The mixture was allowed to warm to rt slowly and stirred at rt overnight. HPLC (1) showed the reaction was complete. The mixture was concentrated and co-evaporated with 1,2-dichloroethane three times to give 2-[(3-methyl-1-phenyl-butyl)-(2,2,2-trifluoroacetyl)amino]acetic acid (3.75 g, 99%) as light yellow oil, which was contaminated by 5% of 1,2-dichloroethane and 7% of TFA. ESI-MS m/z calc. 317.1239, found 316.1 (M−1); Retention time: 2.03 minutes. ¹H NMR (300 MHz, CDCl₃) δ 8.15 (br. s., 1H), 7.47-7.27 (m, 5H), 5.91 (t, J=7.8 Hz, 0.3H), 5.25 (dd, J=10.4, 4.3 Hz, 0.7H), 4.04-3.76 (m, 2H), 2.24-2.04 (m, 1H), 1.90-1.51 (m, 2H), 1.05-0.82 (m, 6H). ¹⁹19F NMR (282 MHz, CDCl₃) δ −67.47 (s, 2.1F), −69.26 (s, 0.9F), LC method K.

### Step 4: 1-Isobutyl-2-(2,2,2-trifluoroacetyl)-1,3-dihydroisoquinolin-4-one

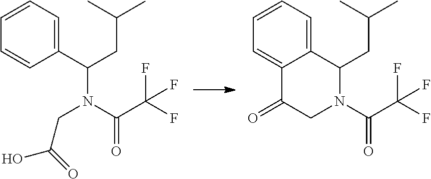

To a solution of 2-[(3-methyl-1-phenyl-butyl)-(2,2,2-trifluoroacetyl)amino]acetic acid (3.55 g, 9.8454 mmol) and DMF (100 mg, 0.1059 mL, 1.3681 mmol) in DCM (50 mL) at 0° C. was added oxalyl chloride (4.26 g, 2.9278 mL, 33.563 mmol) dropwise. The mixture was stirred at rt overnight, concentrated and co-evaporated with 1,2-dichloroethane twice. The residue was dissolved in 1,2-dichloroethane (50 mL), cooled to 0° C. and AlCl3 (3 g, 22.499 mmol) was added. The mixture was stirred at rt for 30 min and at 35° C. for 1.5 h. LCMS (1) showed the reaction was complete. The mixture was slowly added a 2 N aqueous HCl (80 mL) at 0° C. The resulting mixture was extracted with DCM, washed brine and dried with sodium sulfate. Flash chromatography (120 g silica gel, heptanes/EtOAc 0-15%) afforded 1-isobutyl-2-(2,2,2-trifluoroacetyl)-1,3-dihydroisoquinolin-4-one (2.53 g, 86%) as a light-yellow oil. ESI-MS m/z calc. 299.1133, found 300.1 (M+1)⁺; Retention time: 2.14 minutes. ¹H NMR (300 MHz, CDCl₃) δ 8.02 (d, J=7.9 Hz, 1H), 7.67-7.56 (m, 1H), 7.52-7.39 (m, 1H), 7.35-7.21 (m, 1H), 5.87 (dd, J=10.7, 3.7 Hz, 0.7H), 5.23-5.10 (m, 0.6H), 4.64 (dd, J=18.8, 1.2 Hz, 0.7H), 4.27 (d, J=18.8 Hz, 0.7H), 4.05 (d, J=19.7 Hz, 0.3H), 1.97-1.81 (m, 0.7H), 1.79-1.69 (m, 0.6H), 1.66-1.47 (m, 1.7H), 1.10-0.90 (m, 6H). ¹⁹19F NMR (282 MHz, CDCl₃) δ −68.42 (br. s., 0.9F), −68.75 (s, 2.1F), LC method K.

### Step 5: 2,2,2-Trifluoro-1-(4-hydroxy-1-isobutyl-3,4-dihydro-1H-isoquinolin-2-yl)ethanone

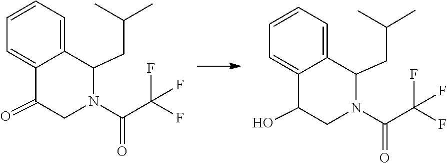 [SMILES: CC(C)CC1c2ccccc2C(=O)CN1C(=O)C(F)(F)F ; CC(C)CC1c2ccccc2C(O)CN1C(=O)C(F)(F)F]

To a solution of 1-isobutyl-2-(2,2,2-trifluoroacetyl)-1,3-dihydroisoquinolin-4-one (2.53 g, 8.4534 mmol) in EtOH (50 mL) at 0° C. was added a solution of sodium borohydride (307 mg, 8.1147 mmol) in EtOH (15 mL) dropwise. The mixture was stirred at 0° C. for 1 h. The mixture was treated with 8.5 mL of 1 N aqueous HCl and concentrated. The residue was partitioned between water and EtOAc. The organic phase was dried with sodium sulfate, filtered and concentrated to give 2,2,2-trifluoro-1-(4-hydroxy-1-isobutyl-3,4-dihydro-1H-isoquinolin-2-yl)ethanone (2.54 g, 100%) as light yellow oil. ESI-MS m/z calc. 301.129, found 302.2 (M+1)⁺; Retention time: 2.03 minutes, LC method K.

### Step 6: 1-Isobutyl-1,2,3,4-tetrahydroisoquinolin-4-ol, major diastereomer 1, and 1-isobutyl-1,2,3,4-tetrahydroisoquinolin-4-ol, minor diastereomer 2

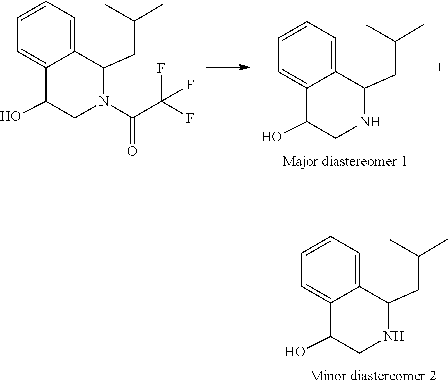 [SMILES: CC(C)CC1c2ccccc2C(O)CN1C(=O)C(F)(F)F ; CC(C)CC1NCC(O)c2ccccc21 ; CC(C)CC1NCC(O)c2ccccc21]

A mixture of 2,2,2-trifluoro-1-(4-hydroxy-1-isobutyl-3,4-dihydro-1H-isoquinolin-2-yl)ethanone (2.54 g, 8.4300 mmol) and ammonia (30 mL of 7 M, 210.00 mmol) in MeOH was stirred at 50° C. overnight. The mixture was concentrated and the residue was purified by flash chromatography (80 g silica gel, DCM (1% NH₃)/MeOH 0-8%) to afford the major diastereomer 1 (less polar on TLC) 1-isobutyl-1,2,3,4-tetrahydroisoquinolin-4-ol (1.173 g, 66%) as a white solid. ESI-MS m/z calc. 205.1467, found 206.2 (M+1)⁺; Retention time: 1.59 minutes. ¹H NMR (300 MHz, CDCl₃) δ 7.40-7.16 (m, 4H), 4.53 (t, J=2.5 Hz, 1H), 3.97 (dd, J=10.0, 3.2 Hz, 1H), 3.26 (dd, J=12.5, 3.1 Hz, 1H), 3.02 (dd, J=12.6, 2.3 Hz, 1H), 2.24 (br. s., 2H), 1.98-1.82 (m, 1H), 1.81-1.69 (m, 1H), 1.67-1.54 (m, 1H), 1.02 (d, J=6.5 Hz, 3H), 0.97 (d, J=6.5 Hz, 3H), and the minor diastereomer 2 (more polar on TLC) 1-isobutyl-1,2,3,4-tetrahydroisoquinolin-4-ol (204 mg, 11%) as off-white solid. ESI-MS m/z calc. 205.1467, found 206.2 (M+1)⁺; Retention time: 1.72 minutes. ¹H NMR (300 MHz, CDCl₃) δ 7.42-7.31 (m, 1H), 7.26-7.17 (m, 2H), 7.10-6.99 (m, 1H), 4.51 (t, J=3.2 Hz, 1H), 3.88 (dd, J=10.9, 3.2 Hz, 1H), 3.26 (dd, J=13.1, 3.1 Hz, 1H), 2.97 (dd, J=13.1, 3.4 Hz, 1H), 2.27 (br. s., 2H), 1.88-1.67 (m, 2H), 1.30 (ddd, J=14.0, 10.3, 3.2 Hz, 1H), 1.00 (d, J=6.5 Hz, 3H), 0.96 (d, J=6.5 Hz, 3H), (LC method G).

### Step 7: 3-[[4-(2,6-Dimethylphenyl)-6-[(1-isobutyl-1,2,3,4-tetrahydroisoquinolin-4-yl)oxy]pyrimidin-2-yl]sulfamoyl]benzoic acid, major diastereomer 1

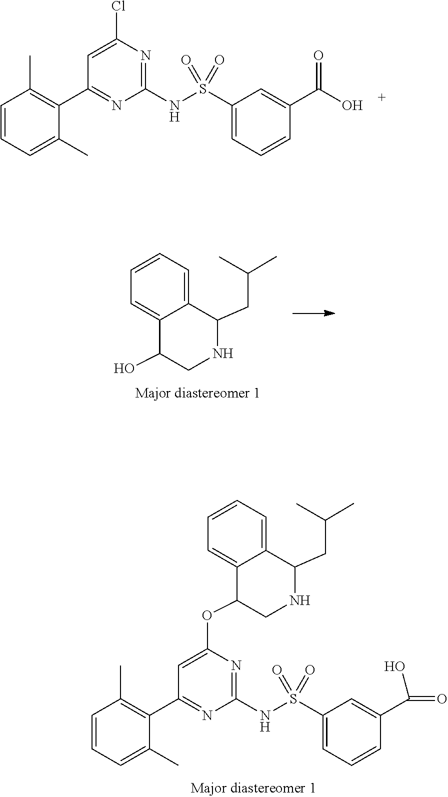 [SMILES: Cc1cccc(C)c1-c1cc(Cl)nc(NS(=O)(=O)c2cccc(C(=O)O)c2)n1 ; CC(C)CC1NCC(O)c2ccccc21 ; Cc1cccc(C)c1-c1cc(OC2CNC(CC(C)C)c3ccccc32)nc(NS(=O)(=O)c2cccc(C(=O)O)c2)n1]

To a solution of 3-[[4-chloro-6-(2,6-dimethylphenyl)pyrimidin-2-yl]sulfamoyl]benzoic acid (104 mg, 0.2489 mmol) and 1-isobutyl-1,2,3,4-tetrahydroisoquinolin-4-ol, major diastereomer 1 (60 mg, 0.2923 mmol) in THE (1 mL) was added sodium tert-butoxide (125 mg, 1.301 mmol) and the reaction mixture was stirred for 16 hours. The solvent was evaporated, the residue was taken up in 1:1 DMSO: MeOH, filtered and purified by HPLC (1-99% ACN in Water (HCl modifier)) to give 3-[[4-(2,6-dimethylphenyl)-6-[(1-isobutyl-1,2,3,4-tetrahydroisoquinolin-4-yl)oxy]pyrimidin-2-yl]sulfamoyl]benzoic acid, major diastereomer 1 (hydrochloride salt)(80 mg, 52%). ESI-MS m/z calc. 586.225, found 587.3 (M+1)⁺; Retention time: 0.49 minutes (LC method D).

### Step 8: 12-(2,6-Dimethylphenyl)-23-(2-methylpropyl)-15-oxa-8λ⁶-thia-1,9,11,25-tetraazapentacyclo[14.7.1.13,7.110,14.017,22]hexacosa-3(26),4,6,10(25),11,13,17,19,21-nonaene-2,8,8-trione, major diastereomer 1 (Compound 190)

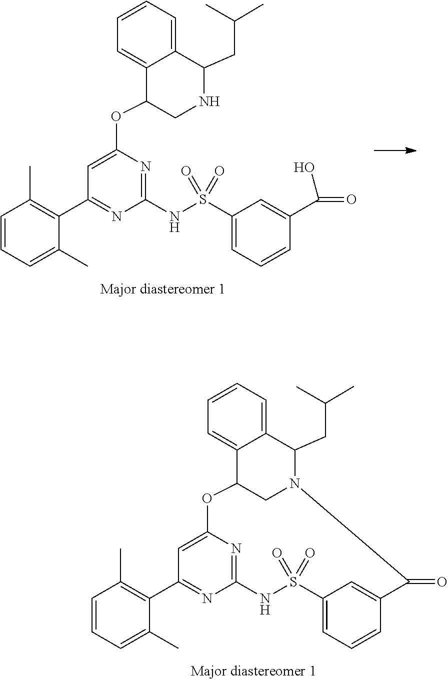 [SMILES: Cc1cccc(C)c1-c1cc(OC2CNC(CC(C)C)c3ccccc32)nc(NS(=O)(=O)c2cccc(C(=O)O)c2)n1 ; Cc1cccc(C)c1-c1cc2nc(n1)NS(=O)(=O)c1cccc(c1)C(=O)N1CC(O2)c2ccccc2C1CC(C)C]

To a solution of 3-[[4-(2,6-dimethylphenyl)-6-[(1-isobutyl-1,2,3,4-tetrahydroisoquinolin-4-yl)oxy]pyrimidin-2-yl]sulfamoyl]benzoic acid, major diastereomer 1 (hydrochloride salt) (27 mg, 0.04602 mmol) and HATU (19.25 mg, 0.05063 mmol) in DMF (0.4 mL) was added DiPEA (40 μL, 0.2296 mmol) and the reaction mixture was stirred at room temperature for 1 hour. The reaction mixture was diluted with MeOH, filtered and purification by HPLC (1-99% ACN in water (HCl modifier)) gave 12-(2,6-dimethylphenyl)-23-(2-methylpropyl)-15-oxa-8λ⁶-thia-1,9,11,25-tetraazapentacyclo[14.7.1.13,7.110,14.017,22]hexacosa-3(26),4,6,10(25),11,13,17,19,21-nonaene-2,8,8-trione, major diastereomer (13.8 mg, 53%). ESI-MS m/z calc. 568.2144, found 569.4 (M+1)⁺; Retention time: 2.0 minutes, LC method A.

### Example 91: Preparation of Compound 191

### Step 1: 3-[[4-(2,6-Dimethylphenyl)-6-[(1-isobutyl-1,2,3,4-tetrahydroisoquinolin-4-yl)oxy]pyrimidin-2-yl]sulfamoyl]benzoic acid, minor diastereomer 2

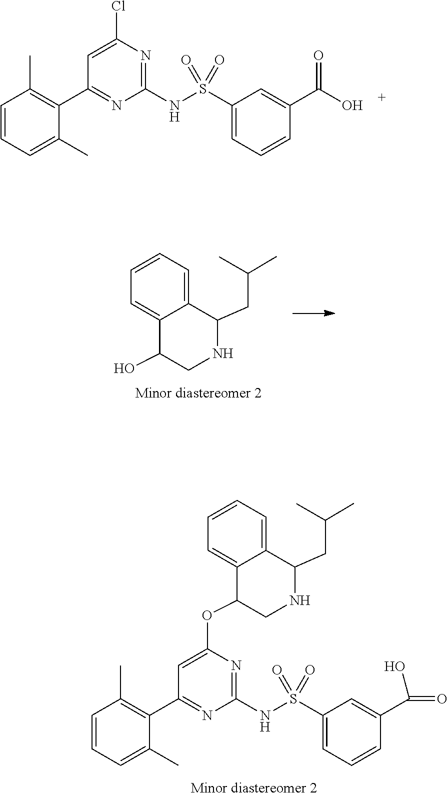 [SMILES: Cc1cccc(C)c1-c1cc(Cl)nc(NS(=O)(=O)c2cccc(C(=O)O)c2)n1 ; CC(C)CC1NCC(O)c2ccccc21 ; Cc1cccc(C)c1-c1cc(OC2CNC(CC(C)C)c3ccccc32)nc(NS(=O)(=O)c2cccc(C(=O)O)c2)n1]

To a solution of 3-[[4-chloro-6-(2,6-dimethylphenyl)pyrimidin-2-yl]sulfamoyl]benzoic acid (104 mg, 0.2489 mmol) and 1-isobutyl-1,2,3,4-tetrahydroisoquinolin-4-ol, minor diastereomer 2 (60 mg, 0.2923 mmol) in THE (1 mL) was added sodium tert-butoxide (125 mg, 1.301 mmol) and the reaction mixture was stirred for 16 hours. The solvent was evaporated, the residue was taken up in DMSO:MeOH (1:1, v:v), filtered, and purified by HPLC (1-99% ACN in water (HCl modifier)) to give 3-[[4-(2,6-dimethylphenyl)-6-[(1-isobutyl-1,2,3,4-tetrahydroisoquinolin-4-yl)oxy]pyrimidin-2-yl]sulfamoyl]benzoic acid (hydrochloride salt), minor diastereomer 2 (80 mg, 52%). ESI-MS m/z calc. 586.225, found 587.3 (M+1)⁺; Retention time: 0.49 minutes, LC method D.

### Step 2: 12-(2,6-Dimethylphenyl)-23-(2-methylpropyl)-15-oxa-8λ⁶-thia-1,9,11,25-tetraazapentacyclo[14.7.1.13,7.110,14.017,22]hexacosa-3(26),4,6,10(25),11,13,17,19,21-nonaene-2,8,8-trione, minor diastereomer 2 (Compound 191)

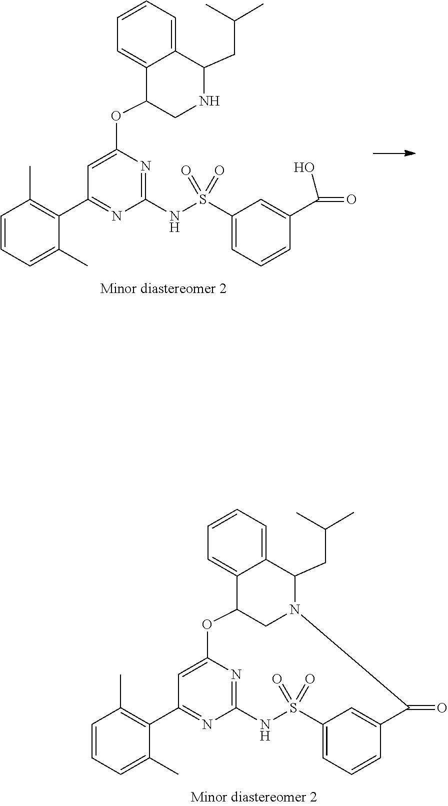 [SMILES: Cc1cccc(C)c1-c1cc(OC2CNC(CC(C)C)c3ccccc32)nc(NS(=O)(=O)c2cccc(C(=O)O)c2)n1 ; Cc1cccc(C)c1-c1cc2nc(n1)NS(=O)(=O)c1cccc(c1)C(=O)N1CC(O2)c2ccccc2C1CC(C)C]

To a solution of 3-[[4-(2,6-dimethylphenyl)-6-[(1-isobutyl-1,2,3,4-tetrahydroisoquinolin-4-yl)oxy]pyrimidin-2-yl]sulfamoyl]benzoic acid (hydrochloride salt) (15 mg, 0.02557 mmol) and HATU (10.70 mg, 0.02814 mmol) in DMF (0.1 mL) was added DiPEA (23 μL, 0.1320 mmol) and the reaction mixture was stirred at room temperature for 1 hour. The reaction mixture was diluted with MeOH, filtered and purification by HPLC (1-99% ACN in water (HCl modifier)) gave 12-(2,6-dimethylphenyl)-23-(2-methylpropyl)-15-oxa-8λ⁶-thia-1,9,11,25-tetraazapentacyclo[14.7.1.13,7.110,14.017,22]hexacosa-3(26),4,6,10(25),11,13,17,19,21-nonaene-2,8,8-trione, minor diastereomer (1.1 mg, 8%). ESI-MS m/z calc. 568.2144, found 569.3 (M+1)⁺; Retention time: 1.94 minutes, LC method A.

### Example 92: Preparation of Compound 192

### Step 1: tert-Butyl N-[(1R)-1-[2-(1,3-dioxolan-2-yl)benzoyl]-3-methyl-butyl]carbamate

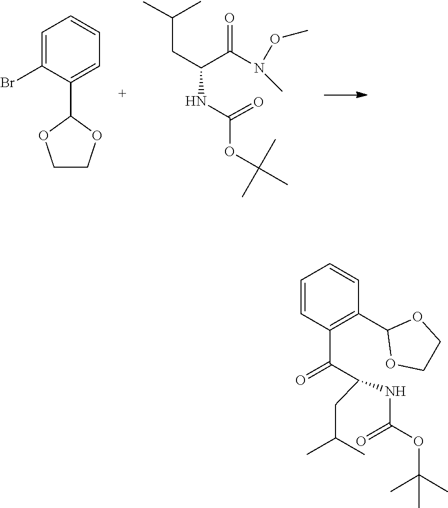 [SMILES: Brc1ccccc1C1OCCO1 ; CON(C)C(=O)[C@@H](CC(C)C)NC(=O)OC(C)(C)C ; CC(C)C[C@@H](NC(=O)OC(C)(C)C)C(=O)c1ccccc1C1OCCO1]

To a nitrogen sparged round bottom flask was added 2-(2-bromophenyl)-1,3-dioxolane (10.6 g, 46.27 mmol) and anhydrous toluene (35 mL). The solution was cooled to 0° C. prior to the dropwise addition of a solution of n-BuLi (18.2 mL of 2.5 M, 45.50 mmol). The reaction mixture was allowed to stir at 0° C. for 15 min prior to addition of tert-butyl N-[(1R)-1-[methoxy(methyl)carbamoyl]-3-methyl-butyl]carbamate (4.24 g, 15.45 mmol) in a toluene solution. The reaction solution was stirred at 0° C. and then at room temp for 2 h. The reaction solution was quenched with aqueous ammonium chloride and diluted with EtOAc. The partitioned EtOAc fraction was dried over sodium sulfate, filtered, concentrated in vacuo and chromatographed on a silica gel column using a gradient of 100% Hexanes to 50% EA/Hexanes over 17 min to afford tert-butyl N-[(1R)-1-[2-(1,3-dioxolan-2-yl)benzoyl]-3-methyl-butyl]carbamate (4.1 g, 73%). ¹H NMR (400 MHz, DMSO-d₆) δ 7.63 (dd, J=7.2, 1.6 Hz, 2H), 7.52 (dtd, J=18.4, 7.3, 1.6 Hz, 2H), 7.23 (d, J=8.1 Hz, 1H), 4.71 (ddd, J=11.3, 8.2, 3.6 Hz, 1H), 4.10-3.99 (m, 2H), 3.99-3.85 (m, 3H), 1.65 (tq, J=10.6, 6.6 Hz, 1H), 1.48 (ddd, J=13.8, 10.9, 4.3 Hz, 1H), 1.34 (s, 10H), 0.83 (t, J=6.9 Hz, 6H).

### Step 2: tert-Butyl N-[(1R)-1-[(R)-[2-(1,3-dioxolan-2-yl)phenyl]-hydroxy-methyl]-3-methyl-butyl]carbamate, and tert-butyl N-[(1R)-1-[(S)-[2-(1,3-dioxolan-2-yl)phenyl]-hydroxy-methyl]-3-methyl-butyl]carbamate

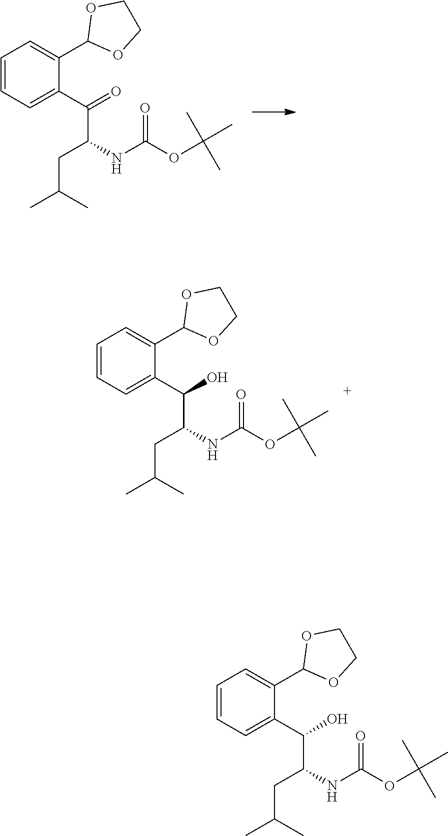 [SMILES: CC(C)C[C@@H](NC(=O)OC(C)(C)C)C(=O)c1ccccc1C1OCCO1 ; CC(C)C[C@@H](NC(=O)OC(C)(C)C)[C@H](O)c1ccccc1C1OCCO1 ; CC(C)C[C@@H](NC(=O)OC(C)(C)C)[C@@H](O)c1ccccc1C1OCCO1]

Into a solution of tert-butyl N-[(1R)-1-[2-(1,3-dioxolan-2-yl)benzoyl]-3-methyl-butyl]carbamate (7.47 g, 20.553 mmol) in anhydrous EtOH (74 mL) was added sodium borohydride (971.95 mg, 25.691 mmol) in several portions at 0° C. The reaction was stirred in an ice bath for 1 hour, then it was quenched with saturated ammonium chloride aqueous solution (70 mL) slowly. The reaction mixture was stirred at room temperature for 15 minutes then it was concentrated under vacuum to remove ethanol. The aqueous residue was extracted with ethyl acetate (3×70 mL). The combined organic phases were washed with brine (70 mL), dried over anhydrous sodium sulfate and concentrated under vacuum. The residue was purified by silica gel chromatography using 0 to 30% hexane-ethyl acetate to give two stereoisomers: tert-Butyl N-[(1R)-1-[(R)-[2-(1,3-dioxolan-2-yl)phenyl]-hydroxy-methyl]-3-methyl-butyl]carbamate (4.804 g, 63%) as a white solid. ¹H NMR (250 MHz, CDCl₃) δ 7.70-7.46 (m, 2H), 7.45-7.20 (m, 2H), 6.06 (s, 1H), 5.07 (s, 1H), 4.84 (d, J=9.5 Hz, 1H), 4.27-3.99 (m, 4H), 1.63 (s, 1H), 1.46-1.19 (m, 11H), 0.86 (dd, J=10.3, 6.5 Hz, 6H), and tert-butyl N-[(1R)-1-[(S)-[2-(1,3-dioxolan-2-yl)phenyl]-hydroxy-methyl]-3-methyl-butyl]carbamate (1.111 g, 15%) as a white solid. ¹H NMR (250 MHz, CDCl₃) δ 7.55 (d, J=7.6 Hz, 2H), 7.46-7.14 (m, 2H), 5.98 (s, 1H), 5.10-4.94 (m, 1H), 4.78 (d, J=9.4 Hz, 1H), 4.20-3.77 (m, 4H), 1.73-1.57 (m, 1H), 1.57-1.47 (m, 1H), 1.32 (s, 8H), 1.12 (s, 2H), 0.90 (dd, J=6.6, 3.8 Hz, 6H).

### Step 3: tert-Butyl (3R,4R)-4-hydroxy-3-isobutyl-3,4-dihydro-1H-isoquinoline-2-carboxylate

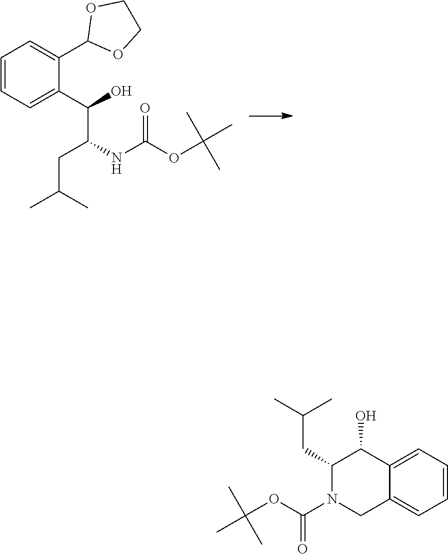 [SMILES: CC(C)C[C@@H](NC(=O)OC(C)(C)C)[C@H](O)c1ccccc1C1OCCO1 ; CC(C)C[C@@H]1[C@H](O)c2ccccc2CN1C(=O)OC(C)(C)C]

tert-Butyl N-[(1R)-1-[(R)-[2-(1,3-dioxolan-2-yl)phenyl]-hydroxy-methyl]-3-methyl-butyl]carbamate (12.45 g, 34.066 mmol) was dissolved in a 4 M solution of HCl in dioxane (85.165 mL). The reaction was stirred at room temperature for 1 h. The solvent was removed under vacuum. The residue was dissolved in EtOH (200 mL). Sodium borohydride (2.5776 g, 68.132 mmol) was added to the reaction mixture at 0° C. The reaction mixture was stirred at the same temperature for 1 hour. The solvent was removed under vacuum. The residue was dissolved in THE (200 mL). To the reaction mixture was added Boc anhydride (14.870 g, 68.132 mmol). The reaction was stirred at room temperature for 2 days. The reaction was quenched with saturated ammonium chloride (100 mL). THE was removed under vacuum. The aqueous solution was extracted with ethyl acetate (3×200 mL). The combined organic phases were washed with brine (100 mL), dried over anhydrous sodium sulfate and concentrated under vacuum. The residue was purified by silica gel chromatography using 0 to 20% hexane-ethyl acetate. The fractions were combined and concentrated. The crude material was further purified by prep-HPLC using 40 to 90% water-acetonitrile (buffered with 0.1% ammonium hydroxide, column XBridge, C18, 10 μm, 5 cm×25 cm, flow rate 50 mL/min, over 40 minutes, 220 nm) to furnish tert-butyl (3R,4R)-4-hydroxy-3-isobutyl-3,4-dihydro-1H-isoquinoline-2-carboxylate (5.35 g, 50%) as a light yellow half solid. ESI-MS m/z calc. 305.1991, found 306.3 (M+1)⁺; Retention time: 2.6 minutes. ¹H NMR (250 MHz, DMSO-d₆) δ 7.54-6.89 (m, 4H), 5.22 (d, J=4.9 Hz, 1H), 4.81 (t, J=17.2 Hz, 1H), 4.41 (d, J=30.4 Hz, 2H), 4.09 (dd, J=29.7, 17.7 Hz, 1H), 1.44 (s, 10H), 0.89 (dd, J=17.8, 6.6 Hz, 8H), LC method W.

### Step 4: di-tert-Butyl (4-chloro-6-(o-tolyl)pyrimidin-2-yl)di-carbamate

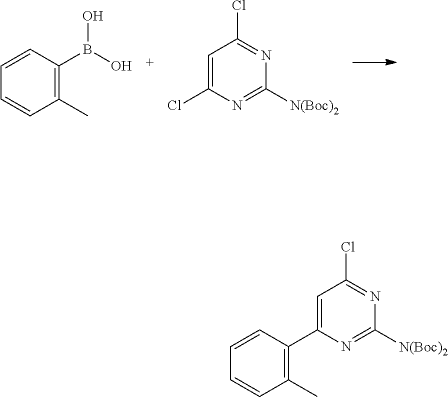

A solution of di-tert-butyl (4,6-dichloropyrimidin-2-yl)di-carbamate (215 g, 0.592 mol) in a mixture of 1,2-dimethoxyethane (1.74 L) and water (455 mL) was degassed for 10 minutes. o-Tolylboronic acid (80.5 g, 0.592 mol), cesium carbonate (482.21 g, 1.48 mol), and Pd(dppf)Cl₂ (21.7 g, 0.0296 mol) were added sequentially to the solution. The reaction was stirred at 65° C. for 1 hour and diluted with water (1 L). The organic layer was separated, and the aqueous layer was extracted with ethyl acetate (2×300 mL). The combined organic layers were dried over sodium sulfate and concentrated to give di-tert-butyl (4-chloro-6-(o-tolyl)pyrimidin-2-yl)di-carbamate (265 g, 106%) as a dark brown viscous oil. ESI-MS m/z calc. 419.16, found 420.5 (M+1)⁺. Retention time: 4.26 minutes.

### Step 5: 4-Chloro-6-(o-tolyl)pyrimidin-2-amine

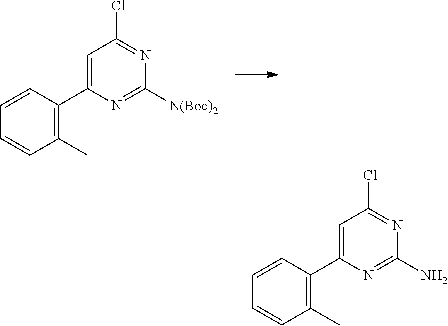

To a solution of di-tert-butyl (4-chloro-6-(o-tolyl)pyrimidin-2-yl)di-carbamate (265 g, 0.632 mol) in dichloromethane (1.8 L) was added slowly 4M hydrogen chloride solution in dioxane (790 mL) and the reaction mixture was allowed to stir for 16 hours at room temperature. The reaction mixture was concentrated and the residue was triturated with 1:1 mixture of dichloromethane:hexanes. The formed solid was collected by filtration, washed with 1:1 mixture of dichloromethane:hexanes and hexanes to give 4-chloro-6-(o-tolyl)pyrimidin-2-amine (hydrochloride salt) (96 g, 63%) as an off white powder. ¹H NMR (250 MHz, DMSO-d₆) δ (ppm): 7.16-7.51 (m, 5H) 6.82 (d, J=1.10 Hz, 1H) 2.37 (s, 3H). ESI-MS m/z calc. 219.06, found 220.3 (M+1)⁺, Retention time: 2.7 minutes.

### Step 6: Methyl 3-(N-(4-chloro-6-(o-tolyl)pyrimidin-2-yl)sulfamoyl)benzoate

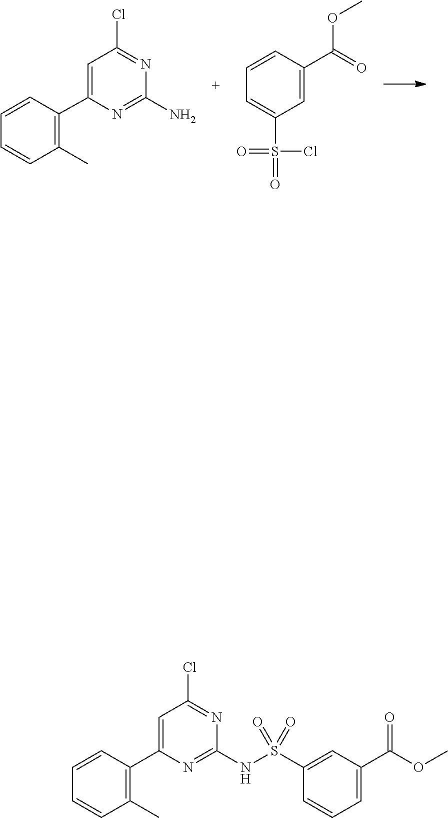 [SMILES: Cc1ccccc1-c1cc(Cl)nc(N)n1 ; COC(=O)c1cccc(S(=O)(=O)Cl)c1 ; COC(=O)c1cccc(S(=O)(=O)Nc2nc(Cl)cc(-c3ccccc3C)n2)c1]

To a mixture of sodium hydride (60% in mineral oil, 3.58 g, 89.50 mmol) in tetrahydrofuran (90 mL) was added a solution of previously freebased 4-chloro-6-(o-tolyl)pyrimidin-2-amine (7.85 g, 35.8 mmol) in tetrahydrofuran (30 mL) at 0° C. This mixture was stirred at room temperature for 1 hour. A solution of methyl 3-(chlorosulfonyl)benzoate (10.09 g, 43.01 mmol) in tetrahydrofuran (30 mL) was slowly added to the mixture at 0° C. and the resulting reaction mixture was stirred at room temperature for 2 hours. The reaction was quenched with 1M aqueous hydrochloric acid to pH=1. The layers were separated, and the aqueous layer was extracted with ethyl acetate (3×100 mL). The combined organic layers were dried over sodium sulfate and reduced to give methyl 3-(N-(4-chloro-6-(o-tolyl)pyrimidin-2-yl)sulfamoyl)benzoate (17.95 g, 119%) as a light brown solid. ESI-MS m/z calc. 417.06 found 418.5 (M+1)⁺, Retention time: 3.44 minutes.

### Step 7: 3-(N-(4-Chloro-6-(o-tolyl)pyrimidin-2-yl)sulfamoyl)benzoic acid

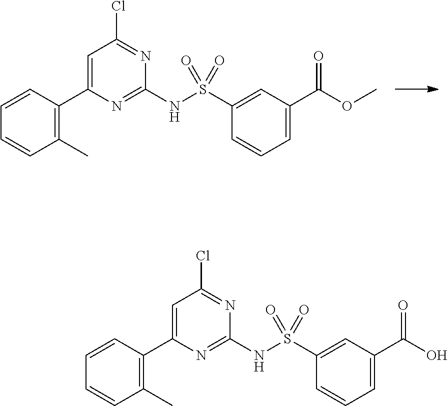 [SMILES: COC(=O)c1cccc(S(=O)(=O)Nc2nc(Cl)cc(-c3ccccc3C)n2)c1 ; Cc1ccccc1-c1cc(Cl)nc(NS(=O)(=O)c2cccc(C(=O)O)c2)n1]

Methyl 3-(N-(4-chloro-6-(o-tolyl)pyrimidin-2-yl)sulfamoyl)benzoate (10.2 g, 24.46 mmol) was dissolved in a mixture of tetrahydrofuran (200 mL) and water (200 mL). Lithium hydroxide (2.93 g, 122.5 mmol) was added and the reaction was stirred to 45° C. for 45 minutes. The reaction was quenched by addition of 1M hydrochloric acid until the pH reached 1. The layers were separated, and the aqueous layer was extracted with ethyl acetate (3×100 mL). The combined organic layers were dried over sodium sulfate and reduced. The residue was purified by reverse phase column chromatography using 50-85% water-acetonitrile (0.1% TFA) to give 3-(N-(4-chloro-6-(o-tolyl)pyrimidin-2-yl)sulfamoyl)benzoic acid (5.12 g, 52%) as a white powder. ¹H NMR (250 MHz, DMSO-d₆) δ (ppm): 8.51 (d, J=1.43 Hz, 1H) 8.13-8.26 (m, 2H) 7.72 (t, J=7.42 Hz, 1H) 7.35-7.45 (m, 2H) 7.22-7.33 (m, 3H) 2.26 (s, 3H). ESI-MS m/z calc. 403.04, found 404.2 (M+1)⁺, Retention time: 2.99 minutes.

### Step 8: (16R,24R)-12-(2-Methylphenyl)-24-(2-methylpropyl)-15-oxa-8λ⁶-thia-1,9,11,25-tetraazapentacyclo[14.7.1.13,7.110,14.017,22]hexacosa-3(26),4,6,10,12,14(25),17,19,21-nonaene-2,8,8-trione (Compound 192)

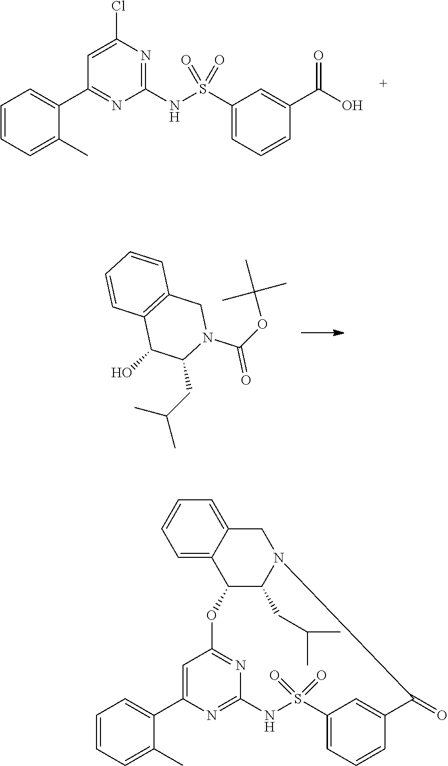 [SMILES: Cc1ccccc1-c1cc(Cl)nc(NS(=O)(=O)c2cccc(C(=O)O)c2)n1 ; CC(C)C[C@@H]1[C@H](O)c2ccccc2CN1C(=O)OC(C)(C)C ; Cc1ccccc1-c1cc2nc(n1)NS(=O)(=O)c1cccc(c1)C(=O)N1Cc3ccccc3[C@@H](O2)[C@H]1CC(C)C]

3-[[4-Chloro-6-(o-tolyl)pyrimidin-2-yl]sulfamoyl]benzoic acid (267 mg, 0.6612 mmol) and tert-butyl (3R,4R)-4-hydroxy-3-isobutyl-3,4-dihydro-1H-isoquinoline-2-carboxylate (200 mg, 0.6549 mmol) were combined and dissolved in tetrahydrofuran (5 mL). Sodium tert-butoxide (248 mg, 2.581 mmol) was added. The reaction mixture was allowed to stir at 50° C. for 2 hours. The reaction mixture was cooled down to room temperature, filtered, and purified by reverse phase preparative chromatography using a C₁₈ column and a 15 min. gradient eluent of 10 to 60% acetonitrile in water containing 5 mM hydrochloric acid to give 3-[[4-[[(3R,4R)-2-tert-butoxycarbonyl-3-isobutyl-3,4-dihydro-1H-isoquinolin-4-yl]oxy]-6-(o-tolyl)pyrimidin-2-yl]sulfamoyl]benzoic acid (286 mg, 64%) ESI-MS m/z calc. 672.2618, found 673.0 (M+1)⁺; Retention time: 1.97 minutes (LC method A).

3-[[4-[[(3R,4R)-2-tert-Butoxycarbonyl-3-isobutyl-3,4-dihydro-1H-isoquinolin-4-yl]oxy]-6-(o-tolyl)pyrimidin-2-yl]sulfamoyl]benzoic acid (286 mg, 64%) in HCl (1 mL of 4 M, 4.000 mmol) was stirred at room temperature for 90 min. The reaction mixture was concentrated to remove solvents. The excess acid was azeotroped with toluene (3×1 mL) to give 3-[[4-[[(3R,4R)-3-isobutyl-1,2,3,4-tetrahydroisoquinolin-4-yl]oxy]-6-(o-tolyl)pyrimidin-2-yl]sulfamoyl]benzoic acid (243 mg, 61%) ESI-MS m/z calc. 572.20935, found 573.0 (M+1)⁺; Retention time: 0.47 minutes (LC method D).

3-[[4-[[(3R,4R)-3-Isobutyl-1,2,3,4-tetrahydroisoquinolin-4-yl]oxy]-6-(o-tolyl)pyrimidin-2-yl]sulfamoyl]benzoic acid (243 mg, 61%), HATU (260 mg, 0.6838 mmol), DIEA (450 μL, 2.584 mmol) and DMF (1 mL) were stirred at room temperature for 30 min. The crude was filtered and purified by reverse phase preparative chromatography using a C₁₈ column and a 15 min gradient eluent of 25 to 75% acetonitrile in water containing 5 mM hydrochloric acid to give (16R,24R)-12-(2-methylphenyl)-24-(2-methylpropyl)-15-oxa-8λ⁶-thia-1,9,11,25-tetraazapentacyclo[14.7.1.13,7.110,14.017,22]hexacosa-3(26),4,6,10,12,14(25),17,19,21-nonaene-2,8,8-trione (31.5 mg, 9%). ¹H NMR (400 MHz, DMSO-d₆) δ 11.82 (s, 1H), 8.45 (s, 1H), 7.90 (d, J=7.8 Hz, 1H), 7.71 (d, J=7.5 Hz, 2H), 7.65 (t, J=7.7 Hz, 1H), 7.55 (s, 1H), 7.45 (s, 2H), 7.39 (d, J=7.0 Hz, 2H), 7.32 (d, J=5.3 Hz, 3H), 5.60 (s, 1H), 5.45 (d, J=18.2 Hz, 1H), 4.28 (d, J=18.1 Hz, 1H), 2.27 (s, 3H), 1.44 (dt, J=13.1, 6.7 Hz, 1H), 1.28 (s, 2H), 0.60 (d, J=6.4 Hz, 3H), 0.35 (d, J=6.4 Hz, 3H). ESI-MS m/z calc. 554.1988, found 555.0 (M+1)⁺; Retention time: 1.78 minutes (LC method A).

### Example 93: Preparation of Compound 193

### Step 1: tert-Butyl (3R,4S)-4-hydroxy-3-isobutyl-3,4-dihydro-1H-isoquinoline-2-carboxylate

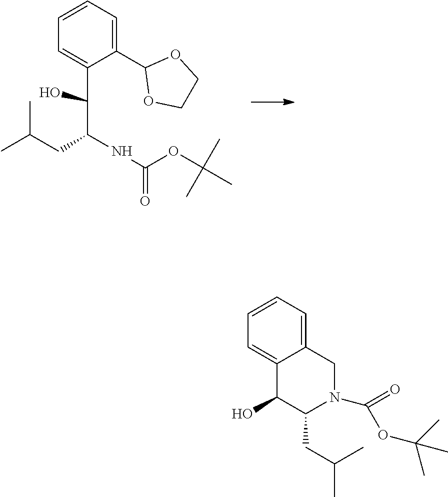 [SMILES: CC(C)C[C@@H](NC(=O)OC(C)(C)C)[C@H](O)c1ccccc1C1OCCO1 ; CC(C)C[C@@H]1[C@@H](O)c2ccccc2CN1C(=O)OC(C)(C)C]

tert-Butyl N-[(1R)-1-[(S)-[2-(1,3-dioxolan-2-yl)phenyl]-hydroxy-methyl]-3-methyl-butyl]carbamate (2.1 g, 5.746 mmol) was treated with dioxane HCl (9.6 mL of 6 M, 57.60 mmol) and stirred at RT for 1 h. After the reaction was complete the mixture was evaporated in vacuo to afford (3R,4S)-3-isobutyl-3,4-dihydroisoquinolin-4-ol (1,150 mg, 98%) ESI-MS m/z calc. 203.13101, found 204.12 (M+1)⁺; Retention time: 0.29 minutes (LC method D). To a solution of the imine in EtOH (30 mL) at 0° C. was added sodium borohydride (435 mg, 11.50 mmol) and the mixture was stirred at RT for 1 h (UPLC control). After completion, the mixture was evaporated in vacuo to afford crude (3R,4S)-3-isobutyl-1,2,3,4-tetrahydroisoquinolin-4-ol (800 mg, 68%) ESI-MS m/z calc. 205.14667, found 206.14 (M+1)⁺; Retention time: 0.32 minutes, (LC method A). The crude amine intermediate was suspended in THE (30 mL) and treated with Boc anhydride (2.5 g, 11.45 mmol). The reaction was stirred at RT overnight, quenched with saturated ammonium chloride and extracted with EtOAc. Organic extract was dried over sodium sulfate, evaporated and purified by Silica Gel chromatography using 40 g column (eluent Hexanes-EtOAc 100-0% to 70-30%) to afford tert-butyl (3R,4S)-4-hydroxy-3-isobutyl-3,4-dihydro-1H-isoquinoline-2-carboxylate (825 mg, 47%)¹H NMR (400 MHz, Chloroform-d) δ 7.35-7.23 (m, 3H), 7.15 (d, J=7.4 Hz, 1H), 5.00 (dd, J=54.0, 17.7 Hz, 1H), 4.81-4.38 (m, 2H), 4.22-4.08 (m, 1H), 1.90 (d, J=8.5 Hz, 1H), 1.51 (s, 9H), 1.26 (t, J=7.1 Hz, 1H), 1.04 (dd, J=9.0, 4.8 Hz, 1H), 0.97 (d, J=6.5 Hz, 3H), 0.89 (d, J=6.7 Hz, 3H). ESI-MS m/z calc. 305.1991, found 232.11 (M+1)⁺; Retention time: 0.71 minutes, LC method D.

### Step 2: (16S,24R)-12-(2-Methylphenyl)-24-(2-methylpropyl)-15-oxa-8λ⁶-thia-1,9,11,25-tetraazapentacyclo[14.7.1.13,7.110,14.017,22]hexacosa-3(26),4,6,10,12,14(25),17,19,21-nonaene-2,8,8-trione (Compound 193)

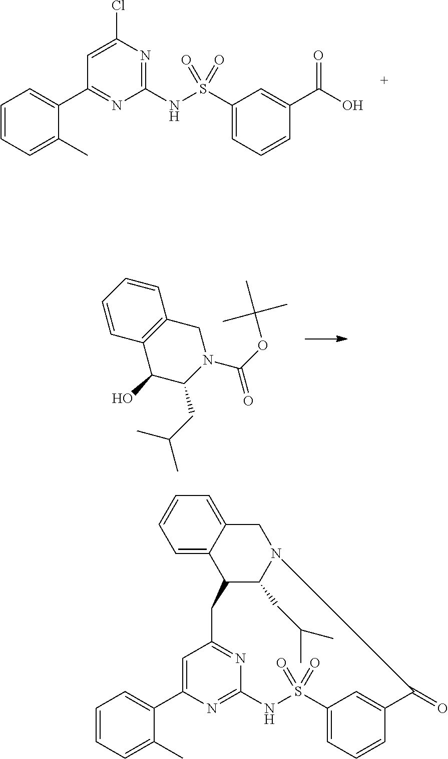

3-[[4-Chloro-6-(o-tolyl)pyrimidin-2-yl]sulfamoyl]benzoic acid (265 mg, 0.6562 mmol) and tert-butyl (3R,4S)-4-hydroxy-3-isobutyl-3,4-dihydro-1H-isoquinoline-2-carboxylate (200 mg, 0.6549 mmol) were combined and dissolved in tetrahydrofuran (5 mL). Sodium tert-butoxide (247 mg, 2.570 mmol) was added, and the reaction mixture was allowed to stir at 50° C. for 2 hours. The reaction mixture was cooled down to room temperature, filtered, and purified by reverse phase preparative chromatography using a C₁₈ column and a 15 min. gradient eluent of 10 to 60 acetonitrile in water containing 5 mM hydrochloric acid to give 3-[[4-[[(3R,4S)-2-tert-butoxycarbonyl-3-isobutyl-3,4-dihydro-1H-isoquinolin-4-yl]oxy]-6-(o-tolyl)pyrimidin-2-yl]sulfamoyl]benzoic acid (256.4 mg, 58%) ¹H NMR (400 MHz, DMSO-d₆) δ 13.25 (s, 1H), 8.33 (s, 1H), 8.03 (d, J=7.4 Hz, 1H), 7.92 (s, 1H), 7.32 (d, J=42.7 Hz, 7H), 7.13 (s, 1H), 6.96 (s, 1H), 6.68 (s, 1H), 6.14 (s, 1H), 4.93 (d, J=17.4 Hz, 1H), 4.68 (s, 1H), 4.20 (s, 1H), 2.33 (s, 3H), 1.50 (s, 10H), 1.21 (s, 2H), 0.90-0.79 (m, 6H). ESI-MS m/z calc. 672.2618, found 673.0 (M+1)⁺; Retention time: 2.19 minutes (LC method A).

3-[[4-[[(3R,4S)-2-tert-Butoxycarbonyl-3-isobutyl-3,4-dihydro-1H-isoquinolin-4-yl]oxy]-6-(o-tolyl)pyrimidin-2-yl]sulfamoyl]benzoic acid (256.4 mg, 58%) in HCl (1 mL of 4 M, 4.000 mmol) was stirred at room temperature for 30 min. The reaction mixture was concentrated and the excess acid was azeotroped with toluene (3×1 mL) to give 3-[[4-[[(3R,4S)-3-isobutyl-1,2,3,4-tetrahydroisoquinolin-4-yl]oxy]-6-(o-tolyl)pyrimidin-2-yl]sulfamoyl]benzoic acid (218.2 mg, 57%) ESI-MS m/z calc. 572.20935, found 573.0 (M+1)⁺; Retention time: 1.07 minutes (LC method A).

The produce, HATU (236.3 mg, 0.6215 mmol), DIEA (450 μL, 2.584 mmol) and DMF (2 mL) were stirred at room temperature for 30 min. The crude was filtered and purified by reverse phase preparative chromatography using a C₁₈ column and a 15 min. gradient eluent of 25 to 75% acetonitrile in water containing 5 mM hydrochloric acid to give (16S,24R)-12-(2-methylphenyl)-24-(2-methylpropyl)-15-oxa-8λ⁶-thia-1,9,11,25-tetraazapentacyclo[14.7.1.13,7.110,14.017,22]hexacosa-3(26),4,6,10,12,14(25),17,19,21-nonaene-2,8,8-trione (64.4 mg, 18%). ¹H NMR (400 MHz, DMSO-d₆) δ 12.22 (s, 1H), 8.60 (s, 1H), 7.99 (d, J=7.7 Hz, 1H), 7.91-7.70 (m, 2H), 7.60 (d, J=6.6 Hz, 1H), 7.54-7.35 (m, 5H), 7.35-7.25 (m, 1H), 6.72 (s, 1H), 6.57 (s, 1H), 5.35-5.18 (m, 1H), 4.56-4.24 (m, 3H), 2.31 (s, 3H), 1.45-1.14 (m, 2H), 0.97-0.79 (m, 1H), 0.66 (d, J=6.5 Hz, 3H), 0.42-0.07 (m, 3H). ESI-MS m/z calc. 554.1988, found 555.0 (M+1)⁺; Retention time: 1.9 minutes, LC method A.

### Example 94: Preparation of Compound 194

### Step 1: tert-Butyl 3,4-dihydro-1H-2,7-naphthyridine-2-carboxylate

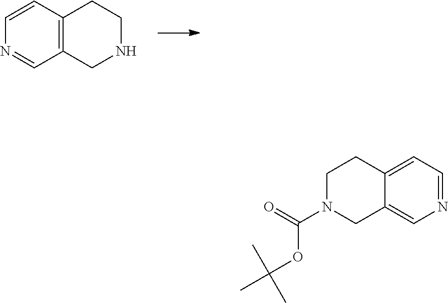 [SMILES: c1cc2c(cn1)CNCC2 ; CC(C)(C)OC(=O)N1CCc2ccncc2C1]

To a suspension of 1,2,3,4-tetrahydro-2,7-naphthyridine (dihydrochloride salt) (6 g, 29.0 mmol) and sodium bicarbonate (7.3 g, 86.9 mmol) in THF/MeOH (100 mL/100 mL) was added di-tert-butyl dicarbonate (7 g, 32.1 mmol) at 0° C. The reaction was stirred at room temperature overnight, and then concentrated in vacuo. The residue was dissolved in water (100 mL), and then extracted with ethyl acetate (100 mL×2). The combined organic phases were washed with brine, dried over anhydrous sodium sulfate, filtered and concentrated in vacuo to give crude tert-butyl 3,4-dihydro-1H-2,7-naphthyridine-2-carboxylate (6.5 g, 96%) as a yellow oil, which was directly used in the next step. ESI-MS m/z calc. 234.1368, found 235.2 (M+1)⁺; Retention time: 1.13 minutes, LC method C.

### Step 2: tert-Butyl 7-oxido-3,4-dihydro-1H-2,7-naphthyridin-7-ium-2-carboxylate

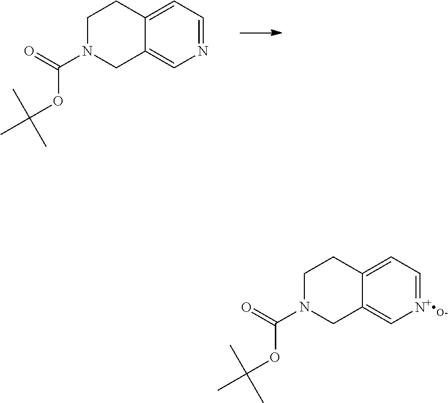

To a solution of tert-butyl 3,4-dihydro-1H-2,7-naphthyridine-2-carboxylate (6.5 g, 27.7 mmol) in dichloromethane (100 mL) was added 3-Chloroperbenzoic acid (7.46 g, 33.29 mmol) at 0° C. The reaction was stirred at room temperature for 2 h, then diluted with dichloromethane (100 mL). The mixture was washed with 10% NaS2O3 (50 mL), 5% aqueous sodium carbonate (100 mL), brine (50 mL), dried over sodium sulfate, filtered and evaporated under reduced pressure. The residue was purified by flash chromatography on silica gel, eluting with mixtures of 0% to 10% methanol in dichloromethane to afford tert-butyl 7-oxido-3,4-dihydro-1H-2,7-naphthyridin-7-ium-2-carboxylate (5.4 g, 78%) as a light-yellow solid. ESI-MS m/z calc. 250.1317, found 251.2 (M+1)⁺; Retention time: 1.38 minutes, LC method C.

### Step 3: tert-Butyl 4-acetoxy-3,4-dihydro-1H-2,7-naphthyridine-2-carboxylate

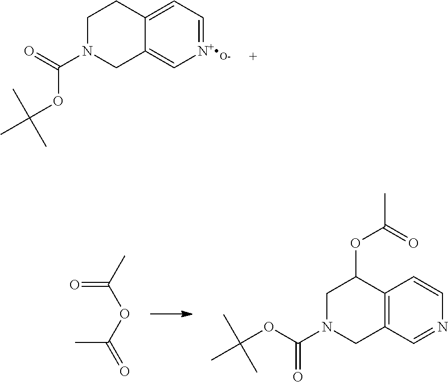

A solution of tert-butyl 7-oxido-3,4-dihydro-1H-2,7-naphthyridin-7-ium-2-carboxylate (5.4 g, 21.6 mmol) in acetic anhydride (44 mL, 466.3 mmol) was heated at 40° C. for 3 h under nitrogen atmosphere. The reaction was cooled. Acetic anhydride was removed by evaporation under reduced pressure. The residue was diluted with EtOAc (150 mL). The mixture was washed with a solution of 5% sodium bicarbonate until basic, then dried over anhydrous sodium sulfate, filtered and evaporated under reduced pressure. The residue was purified by silica gel chromatography, eluting with 0% to 60% ethyl acetate in heptanes to afford tert-butyl 4-acetoxy-3,4-dihydro-1H-2,7-naphthyridine-2-carboxylate (2.0 g, 32%) as a pale-yellow oil. ESI-MS m/z calc. 292.1423, found 293.2 (M+1)⁺; Retention time: 1.46 minutes, LC method C.

### Step 4: tert-Butyl 4-hydroxy-3,4-dihydro-1H-2,7-naphthyridine-2-carboxylate

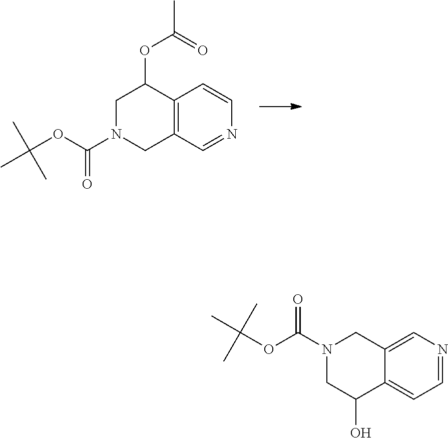 [SMILES: CC(=O)OC1CN(C(=O)OC(C)(C)C)Cc2cnccc21 ; CC(C)(C)OC(=O)N1Cc2cnccc2C(O)C1]

To a solution of tert-butyl 4-acetoxy-3,4-dihydro-1H-2,7-naphthyridine-2-carboxylate (2 g, 6.84 mmol) in methanol (30 mL) was added sodium methoxide solution 25 wt. % in methanol (10.5 mL, 48.6 mmol) at 0° C. The reaction mixture was stirred at room temperature for 2 hours. Most of the solvent was evaporated under reduced pressure at 35° C. The residue was diluted with water (50 mL) and dichloromethane (50 mL). The mixture was stirred for 10 min at room temperature. The two layers were separated. The aqueous layer was extracted with dichloromethane (20 mL). The combined organic layers were washed with water (30 mL), dried over sodium sulfate, filtered and evaporated under reduced pressure. The residue was purified by silica gel chromatography using 30-100% ethyl acetate in heptanes to afford tert-butyl 4-hydroxy-3,4-dihydro-1H-2,7-naphthyridine-2-carboxylate (1.225 g, 71% yield) as a yellow semi solid. ESI-MS m/z calc. 250.1317, found 251.2 (M+1)⁺; Retention time: 1.16 minutes. ¹H NMR (300 MHz, CDCl₃) ppm 1.50 (s, 9H), 3.56 (dd, J=13.2, 6.8 Hz, 1H), 3.91 (dd, J=12.9, 2.6 Hz, 1H), 4.53-4.68 (m, 2H), 4.70-4.81 (m, 1H), 7.44 (d, J=5.0 Hz, 1H), 8.42 (s, 1H), 8.48 (d, J=5.3 Hz, 1H). LC method H.

### Step 5: 12-(2,6-Dimethylphenyl)-15-oxa-8λ⁶-thia-1,9,11,20,25-pentaazapentacyclo[14.7.1.13,7.110,14.017,22]hexacosa-3(26),4,6,10,12,14(25),17,19,21-nonaene-2,8,8-trione (Compound 194)

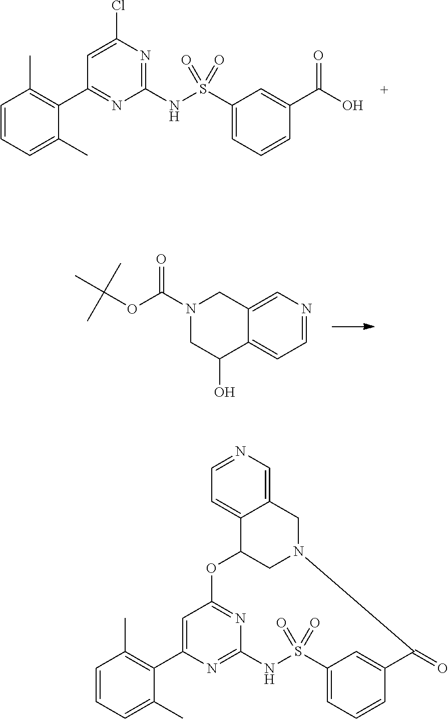 [SMILES: Cc1cccc(C)c1-c1cc(Cl)nc(NS(=O)(=O)c2cccc(C(=O)O)c2)n1 ; CC(C)(C)OC(=O)N1Cc2cnccc2C(O)C1 ; Cc1cccc(C)c1-c1cc2nc(n1)NS(=O)(=O)c1cccc(c1)C(=O)N1Cc3cnccc3C(C1)O2]

3-[[4-Chloro-6-(2,6-dimethylphenyl)pyrimidin-2-yl]sulfamoyl]benzoic acid (200 mg, 0.4786 mmol) and tert-butyl 4-hydroxy-3,4-dihydro-1H-2,7-naphthyridine-2-carboxylate (120 mg, 0.4794 mmol) were combined and dissolved in tetrahydrofuran (1.5 mL). Sodium tert-butoxide (162 mg, 1.686 mmol) was added. The reaction mixture was allowed to stir at 50° C. for 2 hours. The reaction mixture was cooled down to room temperature, filtered, and purified by reverse phase preparative chromatography using a C₁₈ column and a 15 min. gradient eluent of 10 to 60% acetonitrile in water containing 5 mM hydrochloric acid to give 3-[[4-[(2-tert-butoxycarbonyl-3,4-dihydro-1H-2,7-naphthyridin-4-yl)oxy]-6-(2,6-dimethylphenyl)pyrimidin-2-yl]sulfamoyl]benzoic acid (12.1 mg, 4%). ¹H NMR (400 MHz, DMSO-d₆) δ 13.36 (s, 1H), 8.68 (s, 1H), 8.57 (d, J=14.5 Hz, 1H), 8.46 (d, J=3.6 Hz, 1H), 8.13 (s, 2H), 7.56 (d, J=5.2 Hz, 2H), 7.25 (td, J=7.6, 4.9 Hz, 1H), 7.12 (dd, J=7.7, 3.6 Hz, 2H), 6.38 (d, J=24.8 Hz, 1H), 4.47 (d, J=16.0 Hz, 1H), 4.36 (s, 1H), 3.99 (d, J=13.9 Hz, 1H), 2.03 (s, 6H), 1.41 (s, 1H), 1.13 (d, J=20.3 Hz, 5H).

3-[[4-[(2-tert-Butoxycarbonyl-3,4-dihydro-1H-2,7-naphthyridin-4-yl)oxy]-6-(2,6-dimethylphenyl)pyrimidin-2-yl]sulfamoyl]benzoic acid (12.1 mg, 4%) in HCl (1 mL of 4 M, 4.000 mmol) was stirred at room temperature for 30 min. The reaction mixture was concentrated to remove solvents. The excess acid was azeotroped with toluene (3×1 mL) to give 3-(N-(4-(2,6-dimethylphenyl)-6-((1,2,3,4-tetrahydro-2,7-naphthyridin-4-yl)oxy)pyrimidin-2-yl)sulfamoyl)benzoic acid. This product was combined with HATU (160 mg, 0.4208 mmol), DIEA (300 μL, 1.722 mmol) and DMF (1 mL) and the mixture was stirred at room temperature for 30 min. The crude reaction was filtered and purified by reverse phase preparative chromatography using a C₁₈ column and a 15 min. gradient eluent of 1 to 50% acetonitrile in water containing 5 mM hydrochloric acid to give 12-(2,6-dimethylphenyl)-15-oxa-8λ⁶-thia-1,9,11,20,25-pentaazapentacyclo[14.7.1.13,7.110,14.017,22]hexacosa-3(26),4,6,10,12,14(25),17,19,21-nonaene-2,8,8-trione (trifluoroacetate salt) (4.2 mg, 1%)¹H NMR (400 MHz, DMSO-d₆) δ 12.82 (s, 1H), 8.75 (s, 1H), 8.71-8.59 (m, 1H), 7.99 (d, J=7.8 Hz, 1H), 7.85-7.57 (m, 2H), 7.27 (t, J=7.5 Hz, 1H), 7.15 (d, J=7.9 Hz, 2H), 6.51 (d, J=13.9 Hz, 2H), 5.37 (d, J=17.7 Hz, 1H), 4.49 (d, J=17.7 Hz, 1H), 4.13 (dd, J=13.3, 5.0 Hz, 1H), 3.22-3.14 (m, 1H), 2.67 (s, 2H), 2.33 (s, 1H), 2.26-1.93 (m, 5H). ESI-MS m/z calc. 513.1471, found 514.0 (M+1)⁺; Retention time: 0.91 minutes, LC method A.

### Example 95: Preparation of Compound 195 and Compound 196

### Step 1: tert-Butyl 7,8-dihydro-5H-1,6-naphthyridine-6-carboxylate

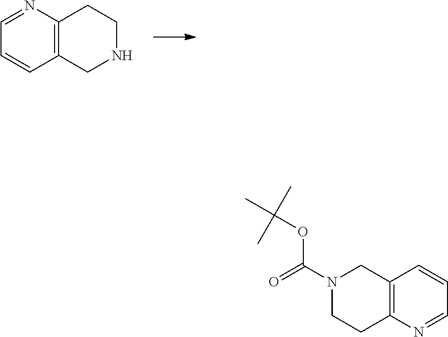 [SMILES: c1cnc2c(c1)CNCC2 ; CC(C)(C)OC(=O)N1CCc2ncccc2C1]

5,6,7,8-Tetrahydro-1,6-naphthyridine (dihydrochloride salt) (10 g, 48.286 mmol) was mixed in THF/MeOH (200 mL/200 mL) at 0° C. Sodium bicarbonate (12.169 g, 144.86 mmol) was added, followed di-tert-butyl dicarbonate (11.065 g, 50.7 mmol). The reaction was stirred at RT overnight, and then concentrated in vacuo. The residue was dissolved in water (˜200 mL), and then extracted with ethyl acetate (150 mL×3). The combined organic phases were washed with brine (100 mL), dried over anhydrous sodium sulfate, filtered and concentrated in vacuo to give tert-butyl 7,8-dihydro-5H-1,6-naphthyridine-6-carboxylate (1.8 g, 106%) as a colorless oil. ESI-MS m/z calc. 234.1368, found 235.4 (M+1)⁺; Retention time: 1.78 minutes, LC method S.

### Step 2: tert-Butyl 1-oxido-7,8-dihydro-5H-1,6-naphthyridin-1-ium-6-carboxylate

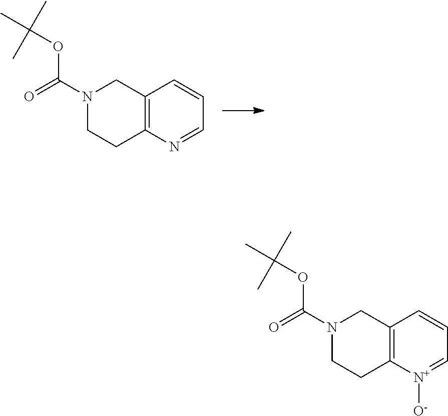 [SMILES: CC(C)(C)OC(=O)N1CCc2ncccc2C1 ; CC(C)(C)OC(=O)N1CCc2c(ccc[n+]2[O-])C1]

tert-Butyl 7,8-dihydro-5H-1,6-naphthyridine-6-carboxylate (12.3 g, 52.498 mmol) was dissolved in dichloromethane (200 mL) and cooled in an ice water bath. m-CPBA (14.495 g, 75% w/w, 62.998 mmol) was added in small portions over 1 min. The reaction was stirred at rt for 5 h, then diluted with dichloromethane (250 mL). The mixture was washed with saturated aqueous sodium carbonate (100 mL×2) followed by brine (100 mL), dried over anhydrous sodium sulfate, filtered and concentrated in vacuo to give tert-butyl 1-oxido-7,8-dihydro-5H-1,6-naphthyridin-1-ium-6-carboxylate (13.5 g, 100.68%) as a white solid. ESI-MS m/z calc. 250.1317, found 251.3 (M+1)⁺; Retention time: 1.74 minutes. ¹H NMR (250 MHz, CDCl₃) ppm 1.48 (s, 9H), 3.05 (t, J=6.0 Hz, 2H), 3.75 (t, J=6.2 Hz, 2H), 4.59 (s, 2H), 6.98-7.09 (m, 1H), 7.10-7.21 (m, 1H), 8.20 (d, J=6.5 Hz, 1H). LC method T.

### Step 3: tert-Butyl 8-hydroxy-7,8-dihydro-5H-1,6-naphthyridine-6-carboxylate

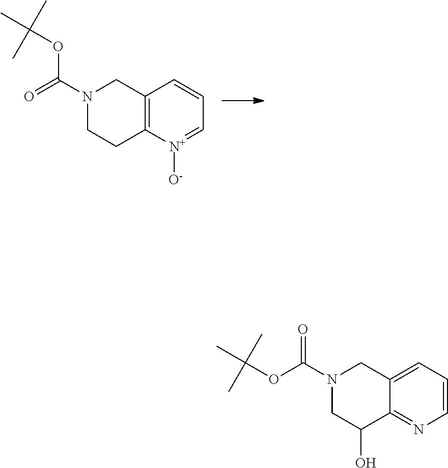 [SMILES: CC(C)(C)OC(=O)N1CCc2c(ccc[n+]2[O-])C1 ; CC(C)(C)OC(=O)N1Cc2cccnc2C(O)C1]

A suspension of tert-butyl 1-oxido-7,8-dihydro-5H-1,6-naphthyridin-1-ium-6-carboxylate (13.5 g, 53.937 mmol) and trifluoracetic anhydride (17.352 g, 11.6 mL, 81.790 mmol) in DCM (175 mL) was stirred at room temperature overnight. Then the reaction mixture was stirred with a solution of sodium hydroxide (1N, 150 mL) for 30 minutes. The resulted mixture was extracted with DCM (125 mL×2). The combined organic phases were washed with brine (100 mL), dried over anhydrous Na2SO4, filtered and concentrated. The residual light brown oil was purified by silica gel chromatography, using 30% to 95% EtOAc in hexanes to afford tert-butyl 8-hydroxy-7,8-dihydro-5H-1,6-naphthyridine-6-carboxylate (9.5 g, 67%) as a light-yellow solid. ¹H NMR (250 MHz, DMSO-d₆) δ 8.46 (d, J=4.1 Hz, 1H), 7.65 (d, J=8.2 Hz, 1H), 7.31 (dd, =7.7,4.7 Hz, 1H), 5.49 (d, J=4.7 Hz, 1H), 4.78-4.30 (m, 3H), 3.77 (dd, J=13.5, 5.5 Hz, 1H), 3.54 (d, J=13.8 Hz, 1H), 1.44 (s, 9H). ESI-MS m/z calc. 250.13174, found 251.1 (M+1)⁺; Retention time: 1.04 minutes; LC method W.

### Step 4: 3-[[4-(2,6-Dimethylphenyl)-6-(5,6,7,8-tetrahydro-1,6-naphthyridin-8-yloxy)pyrimidin-2-yl]sulfamoyl]benzoic acid

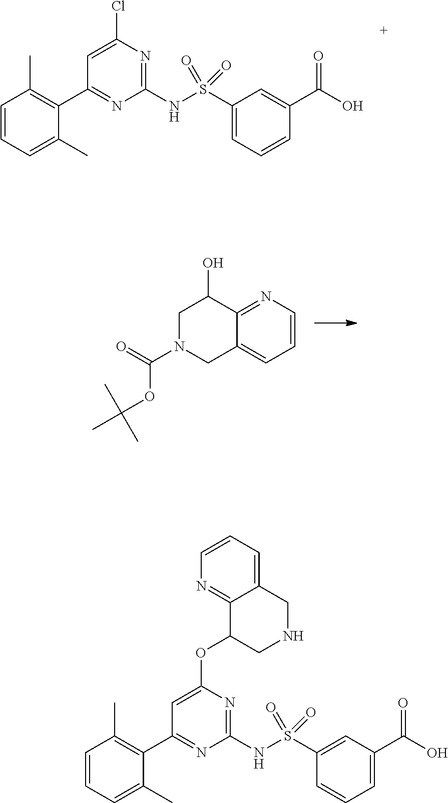 [SMILES: Cc1cccc(C)c1-c1cc(Cl)nc(NS(=O)(=O)c2cccc(C(=O)O)c2)n1 ; CC(C)(C)OC(=O)N1Cc2cccnc2C(O)C1 ; Cc1cccc(C)c1-c1cc(OC2CNCc3cccnc32)nc(NS(=O)(=O)c2cccc(C(=O)O)c2)n1]

To a glass vial was added 3-[[4-chloro-6-(2,6-dimethylphenyl)pyrimidin-2-yl]sulfamoyl]benzoic acid (95 mg, 0.2273 mmol), sodium tert-butoxide (103 mg, 1.072 mmol), and a THE (1 mL) solution of tert-butyl 8-hydroxy-7,8-dihydro-5H-1,6-naphthyridine-6-carboxylate (120 mg, 0.4794 mmol). The reaction was stirred at room temperature for 2 h. The reaction mixture was partitioned between ethyl acetate and a 1M HCl solution. The organics were separated, washed with brine, dried over sodium sulfate and evaporated. to afford crude 3-[[4-[(6-tert-butoxycarbonyl-7,8-dihydro-5H-1,6-naphthyridin-8-yl)oxy]-6-(2,6-dimethylphenyl)pyrimidin-2-yl]sulfamoyl]benzoic acid (83 mg, 58%) ESI-MS m/z calc. 631.2101, found 632.32 (M+1)⁺; Retention time: 0.58 minutes (LC method D).

The product was dissolved in 4M HCl in dioxane (2.1 mL of 4 M, 8.400 mmol) and stirred for 30 min. The reaction mixture was evaporated and the crude material 3-[[4-(2,6-dimethylphenyl)-6-(5,6,7,8-tetrahydro-1,6-naphthyridin-8-yloxy)pyrimidin-2-yl]sulfamoyl]benzoic acid (hydrochloride salt) (85 mg, 66%) ESI-MS m/z calc. 531.15765, found 532.32 (M+1)⁺; Retention time: 0.38 minutes, LC method D.

### Step 5: 12-(2,6-Dimethylphenyl)-15-oxa-8λ⁶-thia-1,9,11,18,25-pentaazapentacyclo[14.7.1.13,7.110,14.017,22]hexacosa-3,5,7(26),10(25),11,13,17,19,21-nonaene-2,8,8-trione

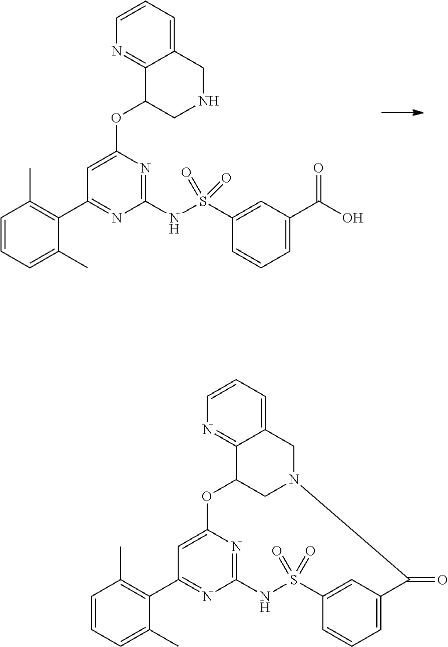 [SMILES: Cc1cccc(C)c1-c1cc(OC2CNCc3cccnc32)nc(NS(=O)(=O)c2cccc(C(=O)O)c2)n1 ; Cc1cccc(C)c1-c1cc2nc(n1)NS(=O)(=O)c1cccc(c1)C(=O)N1Cc3cccnc3C(C1)O2]

To a round bottom flask was added 3-[[4-(2,6-dimethylphenyl)-6-(5,6,7,8-tetrahydro-1,6-naphthyridin-8-yloxy)pyrimidin-2-yl]sulfamoyl]benzoic acid (85 mg, 0.1599 mmol) in DMF (20 mL). To the reaction solution was added [dimethylamino(triazolo[4,5-b]pyridin-3-yloxy)methylene]-dimethyl-ammonium (Phosphorus Hexafluoride Ion) (100 mg, 0.2630 mmol) and DIEA (110 μL, 0.6315 mmol). The reaction was allowed to stir at room temp for 1 hour. The reaction solution was filtered and injected directly onto a Reverse phase HPLC column using a gradient of 1% MeCN in water to 70% MeCN over 15 min to afford purified fractions of 12-(2,6-dimethylphenyl)-15-oxa-8λ⁶-thia-1,9,11,18,25-pentaazapentacyclo[14.7.1.13,7.110,14.017,22]hexacosa-3,5,7(26),10(25),11,13,17,19,21-nonaene-2,8,8-trione (85 mg, 76%) ESI-MS m/z calc. 513.1471, found 514.0 (M+1)⁺; Retention time: 0.43 minutes, LC method D.

### Step 6: 12-(2,6-Dimethylphenyl)-15-oxa-8λ⁶-thia-1,9,11,18,25-pentaazapentacyclo[14.7.1.13,7.110,14.017,22]hexacosa-3,5,7(26),10(25),11,13,17,19,21-nonaene-2,8,8-trione, SFC peak 1 (Compound 196), and 12-(2,6-dimethylphenyl)-15-oxa-8λ⁶-thia-1,9,11,18,25-pentaazapentacyclo[14.7.1.13,7.110,14.017,22]hexacosa-3,5,7(26),10(25),11,13,17,19,21-nonaene-2,8,8-trione, SFC peak 2 (Compound 195)

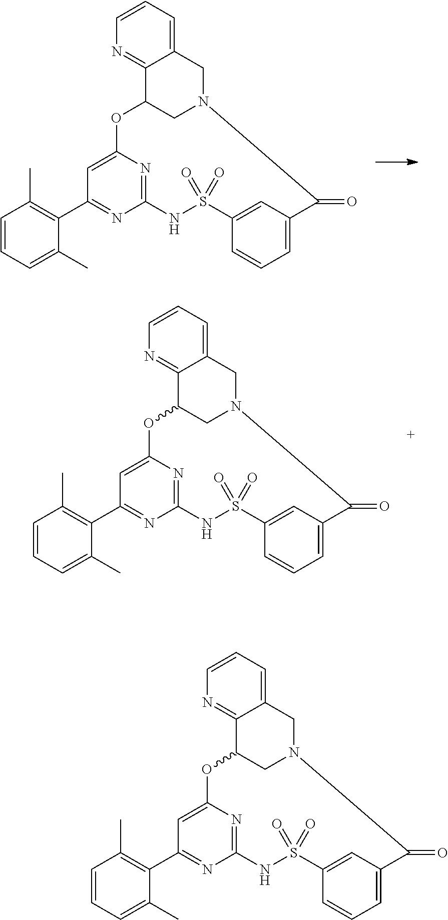 [SMILES: Cc1cccc(C)c1-c1cc2nc(n1)NS(=O)(=O)c1cccc(c1)C(=O)N1Cc3cccnc3C(C1)O2 ; Cc1cccc(C)c1-c1cc2nc(n1)NS(=O)(=O)c1cccc(c1)C(=O)N1Cc3cccnc3C(C1)O2 ; Cc1cccc(C)c1-c1cc2nc(n1)NS(=O)(=O)c1cccc(c1)C(=O)N1Cc3cccnc3C(C1)O2]

Racemic 12-(2,6-dimethylphenyl)-15-oxa-8λ⁶-thia-1,9,11,18,25-pentaazapentacyclo[14.7.1.13,7.110,14.017,22]hexacosa-3,5,7(26),10(25),11,13,17,19,21-nonaene-2,8,8-trione (84 mg, 0.1636 mmol) was dissolved in 1 mL of DMSO and subjected to preparative chiral SFC using a AS column to separate two enantiomers: SFC peak 1, 12-(2,6-dimethylphenyl)-15-oxa-8λ⁶-thia-1,9,11,18,25-pentaazapentacyclo[14.7.1.13,7.110,14.017,22]hexacosa-3,5,7(26),10(25),11,13,17,19,21-nonaene-2,8,8-trione (19 mg, 45%), ¹H NMR (400 MHz, DMSO-d₆) δ 8.65 (s, 1H), 8.58 (dd, J=4.7, 1.6 Hz, 1H), 7.97 (d, J=7.6 Hz, 1H), 7.87 (dd, J=7.9, 1.6 Hz, 1H), 7.78-7.64 (m, 2H), 7.44 (dd, J=7.8, 4.7 Hz, 1H), 7.26 (t, J=7.6 Hz, 1H), 7.13 (d, J=7.6 Hz, 2H), 6.62-6.33 (m, 2H), 5.30 (d, J=17.3 Hz, 1H), 4.61-4.35 (m, 1H), 4.23-4.04 (m, 2H), 2.28-1.83 (m, 6H). ESI-MS m/z calc. 513.1471, found 514.1 (M+1)⁺; Retention time: 1.08 minutes (LC method A); and peak 2, 12-(2,6-dimethylphenyl)-15-oxa-8λ⁶-thia-1,9,11,18,25-pentaazapentacyclo[14.7.1.13,7.110,14.017,22]hexacosa-3,5,7(26),10(25),11,13,17,19,21-nonaene-2,8,8-trione (20 mg, 47%). ESI-MS m/z calc. 513.1471, found 514.1 (M+1)⁺; Retention time: 1.08 minutes, LC method A.

### Example 96: Preparation of Compound 197

### Step 1: Ethyl 4-methylpyridine-3-carboxylate

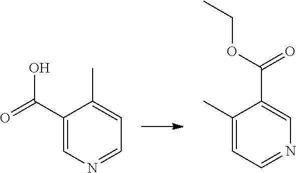

4-Methylpyridine-3-carboxylic acid (30 g, 218.76 mmol) was suspended in ethanol (90 mL) and concentrated sulfuric acid (64.400 g, 35 mL, 656.61 mmol) was added dropwise at room temperature. The solution was heated at reflux for 4 hours before being cooling to rt and poured into ice-water. The solution was basified to pH 9 with NH₃ (aqueous), extracted with diethyl ether (2×100 mL), dried with sodium sulfate and concentrated under reduced pressure to give ethyl 4-methylpyridine-3-carboxylate (25 g, 69%); ¹H NMR (300 MHz, CDCl₃) ppm 1.40 (t, J=7.0 Hz, 3H), 2.61 (s, 3H), 4.38 (q, J=7.0 Hz, 2H), 7.16 (d, J=4.7 Hz, 1H), 8.53 (d, J=5.3 Hz, 1H), 9.05 (s, 1H). ESI-MS m/z calc. 165.079, found 166.2 (M+1)⁺+; Retention time: 1.19 minutes, LC method C.

### Step 2: Ethyl 4-methyl-1-oxido-pyridin-1-ium-3-carboxylate

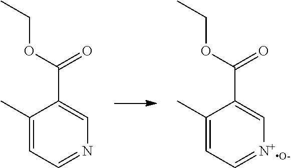

3-Chloroperbenzoic acid (95 g, 423.90 mmol) was added while stirring to a cooled (ice-water bath) solution of ethyl 4-methylpyridine-3-carboxylate (35 g, 211.88 mmol) and the mixture was stirred at room temperature for overnight. chloroform (150 mL) and potassium carbonate (118 g, 853.80 mmol) were added and stirring was continued for 10 min. The mixture was filtered and the filtrate was dried sodium sulfate, filtered, and the solvent was removed in vacuo to give ethyl 4-methyl-1-oxido-pyridin-1-ium-3-carboxylate (31 g, 81%) as a colorless solid; ¹H NMR (300 MHz, CDCl₃) ppm 1.38 (t, J=7.2 Hz, 3H), 2.59 (s, 3H), 4.37 (q, J=7.0 Hz, 2H), 7.15 (d, J=6.5 Hz, 1H), 8.17 (dd, J=6.6, 1.9 Hz, 1H), 8.72 (d, J=1.8 Hz, 1H); ESI-MS m/z calc. 181.0739, found 182.2 (M+1)⁺; Retention time: 1.18 minutes, LC method C.

### Step 3: Ethyl 4-(chloromethyl)pyridine-3-carboxylate

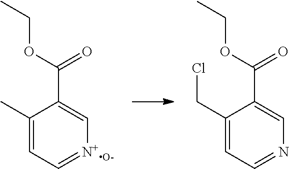

p-Toluenesulfonyl chloride (35 g, 183.59 mmol) was added to a solution of ethyl 4-methyl-1-oxido-pyridin-1-ium-3-carboxylate (15 g, 82.786 mmol) in dioxane (150 mL), the mixture was stirred for 1.5 hours under reflux and then acidified with hydrochloric acid to pH 1. The aqueous layer was washed with diethyl ether (2×75 mL) and the organic extracts were discarded. Saturated aqueous sodium bicarbonate solution (100 mL) and 100 g of sodium bicarbonate were added to the aqueous layer to adjust to pH 8. The aqueous phase was then extracted with diethyl ether (3×75 mL) and the combined organic extracts were dried (sodium sulfate), filtered, and the solvent was evaporated. After column chromatography (120; dichloromethane/ethyl acetate=3:2) to provide ethyl 4-(chloromethyl)pyridine-3-carboxylate (7.7 g, 47%) as a yellow oil; ¹H NMR (300 MHz, CDCl₃) δ 1.42 (t, J=7.0 Hz, 3H), 4.42 (d, J=7.0 Hz, 2H), 5.04 (s, 2H), 7.59 (d, J=5.0 Hz, 1H), 8.74 (d, J=5.0 Hz, 1H), 9.15 (s, 1H). ESI-MS m/z calc. 199.04, found 200.1 (M+1)⁺+; Retention time: 1.68 minutes, LC method C.

### Step 4: tert-Butyl 2-(benzylamino)acetate

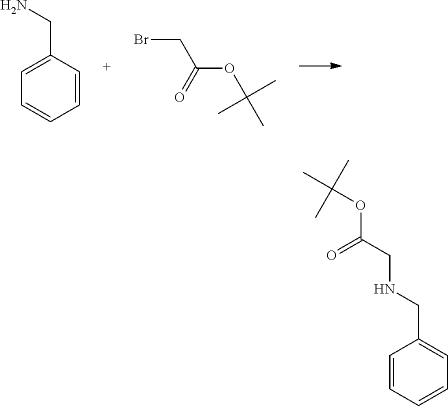

tert-Butyl 2-bromoacetate (30 mL, 194.98 mmol) was added dropwise to a solution of benzylamine (85 mL, 770.42 mmol) in toluene (150 mL) then the mixture was heated at 72° C. for 1 hour. The mixture was poured in 1N sodium hydroxide solution (200 mL) and extracted with ethyl acetate (2×200 mL). The organic phases were combined, washed with water (200 mL) and brine (200 mL), dried over anhydrous sodium sulfate, filtered and concentrated under reduced pressure. The residue was purified by silica-gel column chromatography on a 330 g column, eluting from 0% to 40% of ethyl acetate in hexanes to afford tert-butyl 2-(benzylamino)acetate (39.9 g, 90%). ESI-MS m/z calc. 221.14, found 222.6 (M+1)⁺; Retention time: 1.93 minutes, LC method T.

### Step 5: Ethyl 4-[[benzyl-(2-tert-butoxy-2-oxo-ethyl)amino]methyl]pyridine-3-carboxylate

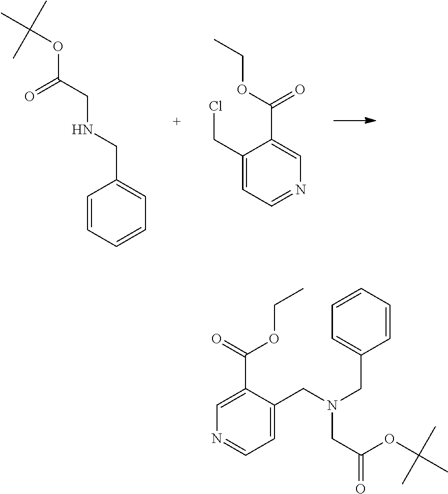 [SMILES: CC(C)(C)OC(=O)CNCc1ccccc1 ; CCOC(=O)c1cnccc1CCl ; CCOC(=O)c1cnccc1CN(CC(=O)OC(C)(C)C)Cc1ccccc1]

tertBbutyl 2-(benzylamino)acetate (3 g, 13.557 mmol), ethyl 4-(chloromethyl)pyridine-3-carboxylate (3 g, 15.028 mmol), DIEA (5.9360 g, 8 mL, 45.929 mmol) and acetonitrile (60 mL) were mixed together at 0° C. The resulting mixture was refluxed 48 hours. Once the reaction was completed the solvent was concentrated under vacuum and the residue was dissolved in DCM (100 mL). The organic phase was washed by saturated sodium bicarbonate (100 mL). The aqueous phase was extracted by DCM (100 mL×2). The organic phase was collected, dried with sodium sulfate and the solvent removed under vacuum to afford a residue which was purified by flash chromatography (120 g, dichloromethane/ethyl acetate: 0 to 20%) to obtain pure ethyl 4-[[benzyl-(2-tert-butoxy-2-oxo-ethyl)amino]methyl]pyridine-3-carboxylate (4 g, 69%) as brownish oil; ¹H NMR (300 MHz, CDCl₃) δ 1.40 (t, J=7.0 Hz, 3H), 1.46 (s, 9H), 3.19 (s, 2H), 3.78 (s, 2H), 4.21 (s, 2H), 4.37 (q, J=7.1 Hz, 2H), 7.20-7.36 (m, 5H), 7.76 (d, J=5.3 Hz, 1H), 8.65 (d, J=5.0 Hz, 1H), 8.98 (s, 1H). ESI-MS m/z calc. 384.2049, found 385.3 (M+1)⁺+; Retention time: 2.23 minutes, LC method C.

### Step 6: tert-Butyl 2-benzyl-4-oxo-1,3-dihydro-2,6-naphthyridine-3-carboxylate

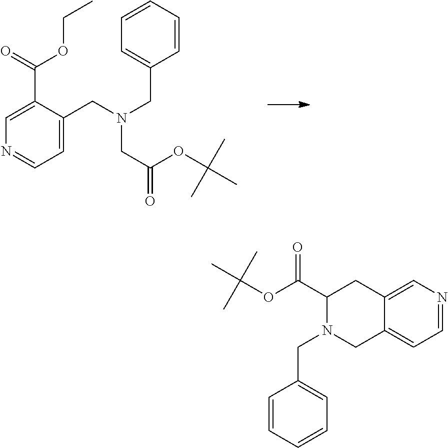 [SMILES: CCOC(=O)c1cnccc1CN(CC(=O)OC(C)(C)C)Cc1ccccc1 ; CC(C)(C)OC(=O)C1Cc2cnccc2CN1Cc1ccccc1]

Potassium tert-butoxide (7 g, 7.7605 mL, 62.382 mmol) in tetrahydrofuran (57 mL) was added dropwise over 5 minutes to a solution of ethyl 4-[[benzyl-(2-tert-butoxy-2-oxo-ethyl)amino]methyl]pyridine-3-carboxylate (7.8 g, 20.288 mmol) in tetrahydrofuran (60 mL) at −78° C. The mixture was stirred at −78° C. for 3 hours. saturated sodium bicarbonate solution (200 mL) was added and the mixture was extracted with ethyl acetate (3×100 mL). The organic phases were combined, washed with brine (200 mL), dried on anhydrous sodium sulfate, filtered and concentrated under reduced pressure to afford tert-butyl 2-benzyl-4-oxo-1,3-dihydro-2,6-naphthyridine-3-carboxylate (6.6 g, 96%) as brownish solid; ¹H NMR (300 MHz, CDCl₃) δ 1.63 (s, 9H), 3.67 (s, 2H), 3.84 (s, 2H), 6.98 (d, J=5.0 Hz, 1H), 7.27-7.42 (m, 5H), 8.59 (d, J=5.0 Hz, 1H), 8.91 (s, 1H), 11.70 (s, 1H). ESI-MS m/z calc. 338.163, found 339.2 (M+1)⁺; Retention time: 2.09 minutes, LC method C.

### Step 7: 2-Benzyl-1,3-dihydro-2,6-naphthyridin-4-one

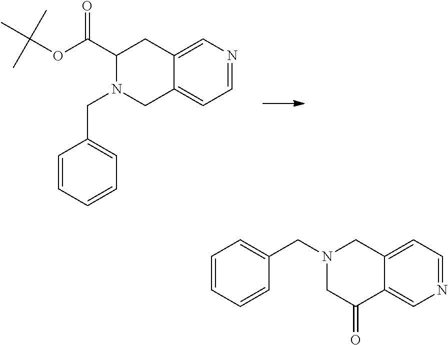 [SMILES: CC(C)(C)OC(=O)C1Cc2cnccc2CN1Cc1ccccc1 ; O=C1CN(Cc2ccccc2)Cc2ccncc21]

Trifluoroacetic acid (66 mL) was added slowly to a solution of tert-butyl 2-benzyl-4-oxo-1,3-dihydro-2,6-naphthyridine-3-carboxylate (6.6 g, 19.504 mmol) in dichloromethane (66 mL) at 0° C. The mixture was stirred at room temperature for 4 hours then poured in saturated sodium bicarbonate solution (300 mL) and then sodium bicarbonate (100 g) was added. The aqueous mixture was extracted with dichloromethane (2×150 mL). The organic phases were combined, dried on anhydrous sodium sulfate, filtered and concentrated under reduced pressure. The residue was purified by silica-gel column chromatography on a 80 g column, eluting from 0% to 70% of ethyl acetate in heptanes to afford 2-benzyl-1,3-dihydro-2,6-naphthyridin-4-one (1.95 g, 42%) as yellow solid; ¹H NMR (300 MHz, CDCl₃) δ 3.43 (d, J=0.9 Hz, 2H), 3.76 (s, 2H), 3.78 (s, 2H), 7.14 (d, J=5.0 Hz, 1H), 7.28-7.41 (m, 5H), 8.66 (d, J=5.3 Hz, 1H), 9.18 (s, 1H). ESI-MS m/z calc. 238.1106, found 239.2 (M+1)⁺+; Retention time: 1.42 minutes, LC method C.

### Step 8: 2-Benzyl-3,4-dihydro-1H-2,6-naphthyridin-4-ol

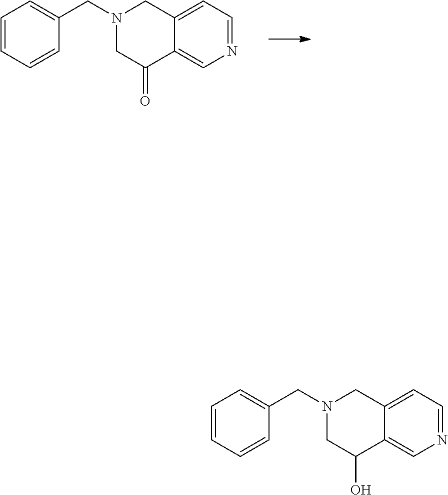 [SMILES: O=C1CN(Cc2ccccc2)Cc2ccncc21 ; OC1CN(Cc2ccccc2)Cc2ccncc21]

Sodium borohydride (930 mg, 24.582 mmol) was added to a solution of 2-benzyl-1,3-dihydro-2,6-naphthyridin-4-one (1.95 g, 8.1835 mmol) in methanol (39 mL) at 0° C. The mixture was stirred at 0° C. for 2 h. Acetone was added and the reaction was concentrated under reduced pressure. The residue was taken with ethyl acetate (50 mL) and saturated sodium bicarbonate solution (75 mL). The phases were separated, and the aqueous phase was extracted with ethyl acetate (2×50 mL). The organic phases were combined, washed with brine (100 mL), dried on anhydrous sodium sulfate, filtered and concentrated under reduced pressure to afford 2-benzyl-3,4-dihydro-1H-2,6-naphthyridin-4-ol (1.8 g, 92%) as brownish oil; ¹H NMR (300 MHz, CDCl₃) δ 2.61-2.73 (m, 1H), 3.06-3.18 (m, 1H), 3.38 (d, J=16.4 Hz, 1H), 3.70-3.87 (m, 3H), 4.69 (br. s., 1H), 6.93 (d, J=5.0 Hz, 1H), 7.28-7.44 (m, 5H), 8.39 (d, J=5.3 Hz, 1H), 8.64 (s, 1H). ESI-MS m/z calc. 240.1263, found 241.2 (M+1)⁺; Retention time: 0.5 minutes, LC method C.

### Step 9: 1,2,3,4-Tetrahydro-2,6-naphthyridin-4-ol

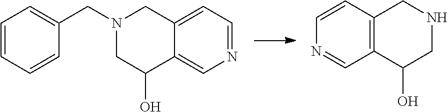 [SMILES: OC1CN(Cc2ccccc2)Cc2ccncc21 ; OC1CNCc2ccncc21]

Palladium on carbon (800 mg, 10% w/w, 0.7517 mmol) was added to a solution of 2-benzyl-3,4-dihydro-1H-2,6-naphthyridin-4-ol (1.8 g, 7.4906 mmol) and hydrochloric acid (16 mL of 10% w/v, 43.883 mmol) in ethanol (54 mL). The mixture was placed under hydrogen atmosphere and stirred for 3 hours. Hydrogen was removed and the mixture was filtered on Celite and the filtrate was concentrated under reduced pressure to afford 1,2,3,4-tetrahydro-2,6-naphthyridin-4-ol (hydrochloride salt) (1.5 g, 97%) as pale-yellow solid; ¹H NMR (300 MHz, DMSO-d₆) δ 3.09-3.66 (m, 2H), 4.20-4.82 (m, 2H), 5.14 (br. s., 1H), 7.93 (d, J=5.3 Hz, 1H), 8.82 (d, J=5.0 Hz, 1H), 9.00 (s, 1H), 9.97 (br. s., 1H), 10.72 (br. s., 1H). ESI-MS m/z calc. 150.0793, found 151.2 (M+1)⁺+; Retention time: 0.26 minutes, LC method C.

### Step 10: 12-(2,6-Dimethylphenyl)-15-oxa-8λ⁶-thia-1,9,11,19,25-pentaazapentacyclo[14.7.1.13,7.110,14.017,22]hexacosa-3(26),4,6,10,12,14(25),17,19,21-nonaene-2,8,8-trione (Compound 197)

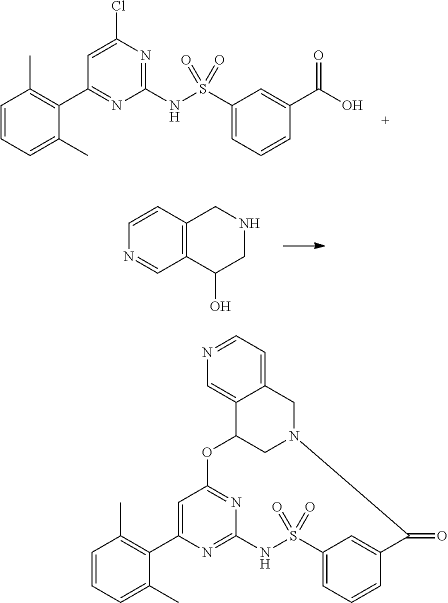

3-[[4-Chloro-6-(2,6-dimethylphenyl)pyrimidin-2-yl]sulfamoyl]benzoic acid (113 mg, 0.2704 mmol) and 1,2,3,4-tetrahydro-2,6-naphthyridin-4-ol (hydrochloride salt) (54 mg, 0.2893 mmol) were combined and dissolved in tetrahydrofuran (1 mL). Sodium tert-butoxide (105 mg, 1.093 mmol) was added. The reaction mixture was allowed to stir at 50° C. for 2 hours. The reaction mixture was cooled down to room temperature, filtered, and purified by reverse phase preparative chromatography using a C₁₈ column and a 15 min. gradient eluent of 10 to 60% acetonitrile in water containing 5 mM hydrochloric acid to give 3-[[4-(2,6-dimethylphenyl)-6-(1,2,3,4-tetrahydro-2,6-naphthyridin-4-yloxy)pyrimidin-2-yl]sulfamoyl]benzoic acid (12.5 mg, 8%). ¹H NMR (400 MHz, DMSO-d₆) δ 13.16 (s, 1H), 10.51 (s, 1H), 9.46 (s, 1H), 8.91 (s, 1H), 8.70 (s, 1H), 8.47 (s, 1H), 8.16 (s, 2H), 7.64 (d, J=31.6 Hz, 2H), 7.27 (s, 1H), 7.11 (s, 2H), 6.28 (d, J=17.2 Hz, 2H), 4.48 (t, J=19.2 Hz, 2H), 4.08 (s, 1H), 2.02 (s, 6H). ESI-MS m/z calc. 531.15765, found 532.0 (M+1)⁺; Retention time: 0.75 minutes (LC method A).

3-[[4-(2,6-Dimethylphenyl)-6-(1,2,3,4-tetrahydro-2,6-naphthyridin-4-yloxy)pyrimidin-2-yl]sulfamoyl]benzoic acid (12.5 mg, 8%), diisopropyl ethyl amine (50 μL, 0.2871 mmol), HATU (105 mg, 0.2761 mmol), and DMF (1 mL) were stirred at room temperature for 30 min. The crude was filtered and purified by reverse phase preparative chromatography using a C₁₈ column and a 15 min. gradient eluent of 1 to 50% acetonitrile in water containing 5 mM hydrochloric acid to give 12-(2,6-dimethylphenyl)-15-oxa-8λ⁶-thia-1,9,11,19,25-pentaazapentacyclo[14.7.1.13,7.110,14.017,22]hexacosa-3(26),4,6,10,12,14(25),17,19,21-nonaene-2,8,8-trione (4.6 mg, 3%) ESI-MS m/z calc. 513.1471, found 514.0 (M+1)⁺; Retention time: 0.92 minutes, LC method A.

### Example 97: Preparation of Compound 198

### Step 1: Ethyl 2-(bromomethyl)pyridine-3-carboxylate

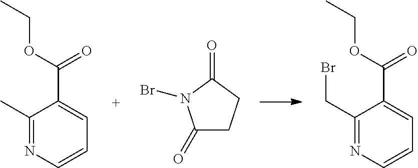

To a solution of ethyl 2-methylpyridine-3-carboxylate (25 g, 151.34 mmol) and AIBN (550 mg, 3.35 mmol) in carbon tetrachloride (300 mL) and acetic acid (8.7 mL) was added N-bromosuccinimide (52 g, 292.16 mmol) at room temperature. The mixture was stirred in an oil bath at 80° C. for 8 hours. The solution was cooled and poured in saturated sodium bicarbonate (600 mL) and extracted with dichloromethane (3×400 mL). The organic phase was washed with brine (800 mL), dried over anhydrous sodium sulfate and concentrated. The residue was purified by silica gel column chromatography using 0-50% hexane-ethyl acetate to afford ethyl 2-(bromomethyl)pyridine-3-carboxylate (40.42 g, 93%). ESI-MS m/z calc. 243.0, found 244.3 (M+1)⁺; Retention time: 2.43 minutes, LC method T.

### Step 2: Ethyl 2-[[benzyl-(2-tert-butoxy-2-oxo-ethyl)amino]methyl]pyridine-3-carboxylate

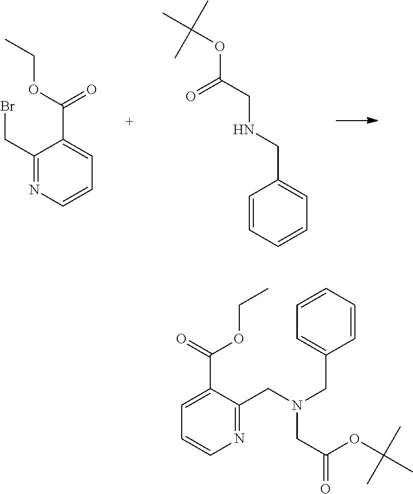

tert-Butyl 2-(benzylamino)acetate (26.4 g, 118.82 mmol) was added to a solution of ethyl 2-(bromomethyl)pyridine-3-carboxylate (28 g, 108.98 mmol) and triethylamine (50 mL, 358.73 mmol) in tetrahydrofuran (300 mL) and the mixture was stirred overnight at room temperature. Water (1500 mL) was added and was extracted with ethyl acetate (2×1000 mL). The organic phases were combined, washed with brine (800 mL), dried over anhydrous sodium sulfate, filtered and concentrated under reduced pressure. The residue was purified by silica-gel column chromatography, eluting from 0% to 30% of ethyl acetate in hexanes to afford ethyl 2-[[benzyl-(2-tert-butoxy-2-oxo-ethyl)amino]methyl]pyridine-3-carboxylate (34.55 g, 70%) as an orange oil. ESI-MS m/z calc. 384.20, found 385.6 (M+1)⁺; Retention time: 2.77 minutes, LC method T.

### Step 3: tert-Butyl 7-benzyl-5-oxo-6,8-dihydro-1,7-naphthyridine-6-carboxylate

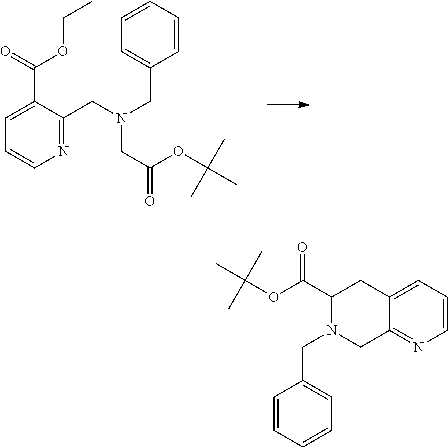 [SMILES: CCOC(=O)c1cccnc1CN(CC(=O)OC(C)(C)C)Cc1ccccc1 ; CC(C)(C)OC(=O)C1Cc2cccnc2CN1Cc1ccccc1]

Potassium tert-butoxide (20 g, 178.23 mmol) in tetrahydrofuran (200 mL) was added dropwise over 30 minutes to a solution of ethyl 2-[[benzyl-(2-tert-butoxy-2-oxo-ethyl)amino]methyl]pyridine-3-carboxylate (34.55 g, 76.38 mmol) in tetrahydrofuran (350 mL) at −78° C. The mixture was stirred at −78° C. for 2 hours and then kept in a freezer overnight. Saturated sodium bicarbonate solution (900 mL) was added and the mixture was extracted with ethyl acetate (3×600 mL). The organic phases were combined, washed with brine (800 mL), dried on anhydrous sodium sulfate, filtered and concentrated under reduced pressure. The residue was purified by silica-gel column chromatography, eluting from 0% to 30% of ethyl acetate in hexane to afford tert-butyl 7-benzyl-5-oxo-6,8-dihydro-1,7-naphthyridine-6-carboxylate (19.76 g, 75%) ESI-MS m/z calc. 338.163, found 339.5 (M+1)⁺; Retention time: 2.97 minutes, LC method T.

### Step 4: 7-Benzyl-6,8-dihydro-1,7-naphthyridin-5-one

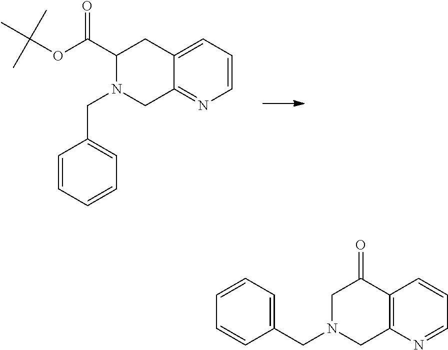 [SMILES: CC(C)(C)OC(=O)C1Cc2cccnc2CN1Cc1ccccc1 ; O=C1CN(Cc2ccccc2)Cc2ncccc21]

Trifluoroacetic acid (150 mL) was added slowly to a solution of tert-butyl 7-benzyl-5-oxo-6,8-dihydro-1,7-naphthyridine-6-carboxylate (19.26 g, 55.78 mmol) in dichloromethane (80 mL) at 0° C. The mixture was stirred at room temperature for 3 hours. This TFA reaction solution was basified to PH-14 by 2N sodium hydroxide. The aqueous phase was extracted with dichloromethane (3×200 mL). The organic phase was washed with brine (500 mL), dried over sodium sulfate and concentrated. The crude product of 7-benzyl-6,8-dihydro-1,7-naphthyridin-5-one (14.2 g, 78%) ESI-MS m/z calc. 238.1106, found 239.3 (M+1)⁺; Retention time: 1.87 minutes, LC method S.

### Step 5: 7-Benzyl-6,8-dihydro-5H-1,7-naphthyridin-5-ol

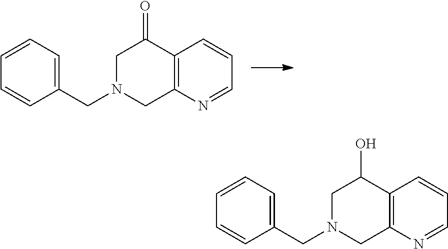 [SMILES: O=C1CN(Cc2ccccc2)Cc2ncccc21 ; OC1CN(Cc2ccccc2)Cc2ncccc21]

Sodium borohydride (3.5 g, 90.66 mmol) was added to a solution of 7-benzyl-6,8-dihydro-1,7-naphthyridin-5-one (14.2 g, 43.50 mmol) in methanol (100 mL) at 0° C. The mixture was stirred at room temperature for 30 minutes. Add acetone and concentrate under reduced pressure. The residue was purified by silica-gel column chromatography eluting from 0% to 50% of 7-benzyl-6,8-dihydro-5H-1,7-naphthyridin-5-ol (5.78 g purity 85%+7.65 g purity 95%, 99%). ESI-MS m/z calc. 240.13, found 241.4 (M+1)⁺; Retention time: 1.39 minutes, LC method S.

### Step 6: 5,6,7,8-Tetrahydro-1,7-naphthyridin-5-ol

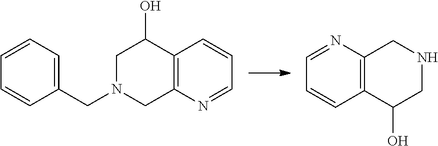 [SMILES: OC1CN(Cc2ccccc2)Cc2ncccc21 ; OC1CNCc2ncccc21]

To a solution of 7-benzyl-6,8-dihydro-5H-1,7-naphthyridin-5-ol (1.18 g, 4.52 mmol) in methanol (30 mL) and acetic acid (1.5 mL) was added 10% palladium on carbon (400 mg). The mixture was stirred under hydrogen atmosphere at 50 psi for 3 hours. The reaction mixture was filtered through Celite pad. The filtrate was concentrated to give 5,6,7,8-tetrahydro-1,7-naphthyridin-5-ol (735 mg, 103%) ESI-MS m/z calc. 150.0793, found 151.3 (M+1)⁺; Retention time: 0.61 minutes, LC method S.

### Step 7: tert-Butyl 5-hydroxy-6,8-dihydro-5H-1,7-naphthyridine-7-carboxylate

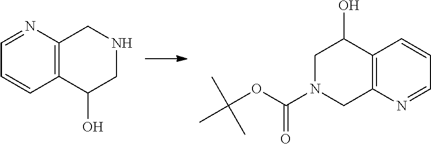 [SMILES: OC1CNCc2ncccc21 ; CC(C)(C)OC(=O)N1Cc2ncccc2C(O)C1]

To a solution of 5,6,7,8-tetrahydro-1,7-naphthyridin-5-ol (700 mg, 4.44 mmol) in tetrahydrofuran (20 mL) and triethylamine (3.5 mL, 25.11 mmol) was added tert-butoxycarbonyl tert-butyl carbonate (1.5 g, 6.87 mmol) at 0° C. The reaction mixture was stirred at room temperature for 2.5 hours. The reaction mixture were treated with ethyl acetate (2×100 mL) and saturated sodium bicarbonate (150 mL). After work-up, the residue was purified by silica gel column chromatography using 0-10% methanol in dichloromethane to afford tert-butyl 5-hydroxy-6,8-dihydro-5H-1,7-naphthyridine-7-carboxylate (1.12 g, 92%) ESI-MS m/z calc. 250.1317, found 251.1 (M+1)⁺; Retention time: 1.97 minutes, LC method S.

### Step 8: 3-[[4-[(7-tert-Butoxycarbonyl-6,8-dihydro-5H-1,7-naphthyridin-5-yl)oxy]-6-(2,6-dimethylphenyl)pyrimidin-2-yl]sulfamoyl]benzoic acid

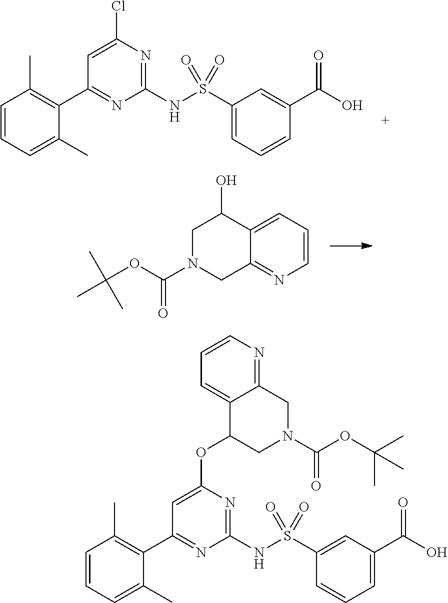

To a solution of 3-[[4-chloro-6-(2,6-dimethylphenyl)pyrimidin-2-yl]sulfamoyl]benzoic acid (125 mg, 0.2991 mmol) and tert-butyl 5-hydroxy-6,8-dihydro-5H-1,7-naphthyridine-7-carboxylate (65 mg, 0.2597 mmol) in NMP (0.75 mL) was added NaH (44 mg of 60% w/w, 1.100 mmol) and the reaction mixture was stirred at 50° C. for 16 hours, then at 100° C. for 10 min. The reaction mixture was cooled, poured into water, the pH brought to 6 with 1N HCl and then extracted with EtOAc (2×). The organics were combined, dried over sodium sulfate and evaporated to dryness. Purification by column chromatography (16 g silica; 0-50% EtOAc in hexanes) gave 3-[[4-[(7-tert-butoxycarbonyl-6,8-dihydro-5H-1,7-naphthyridin-5-yl)oxy]-6-(2,6-dimethylphenyl)pyrimidin-2-yl]sulfamoyl]benzoic acid (60 mg, 37%) as a red glass. ESI-MS m/z calc. 631.2101, found 632.4 (M+1)⁺; Retention time: 0.57 minutes, LC method D.

### Step 9: 3-[[4-(2,6-Dimethylphenyl)-6-(5,6,7,8-tetrahydro-1,7-naphthyridin-5-yloxy)pyrimidin-2-yl]sulfamoyl]benzoic acid

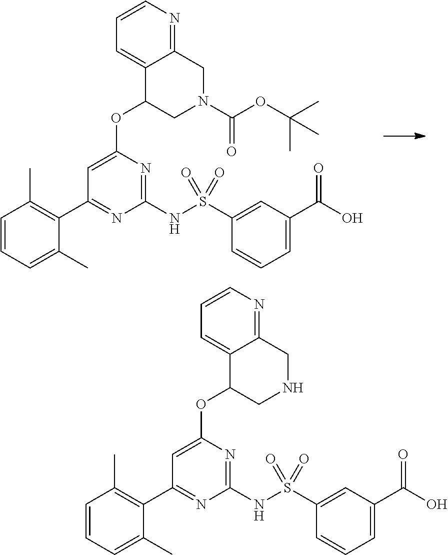

To a solution of 3-[[4-[(7-tert-butoxycarbonyl-6,8-dihydro-5H-1,7-naphthyridin-5-yl)oxy]-6-(2,6-dimethylphenyl)pyrimidin-2-yl]sulfamoyl]benzoic acid (60 mg, 0.09498 mmol) in MeOH (1 mL) was added HCl (4M in dioxane) (1 mL of 4 M, 4.000 mmol) and the reaction mixture was stirred at room temperature for 1 h then evaporated to dryness to give 3-[[4-(2,6-dimethylphenyl)-6-(5,6,7,8-tetrahydro-1,7-naphthyridin-5-yloxy)pyrimidin-2-yl]sulfamoyl]benzoic acid (hydrochloride salt) (34 mg, 63%) as a white solid. ESI-MS m/z calc. 531.15765, found 532.3 (M+1)⁺; Retention time: 0.38 minutes, LC method D.

### Step 10: 12-(2,6-Dimethylphenyl)-15-oxa-8λ⁶-thia-1,9,11,21,25-pentaazapentacyclo[14.7.1.13,7.110,14.017,22]hexacosa-3,5,7(26),10(25),11,13,17,19,21-nonaene-2,8,8-trione (Compound 198)

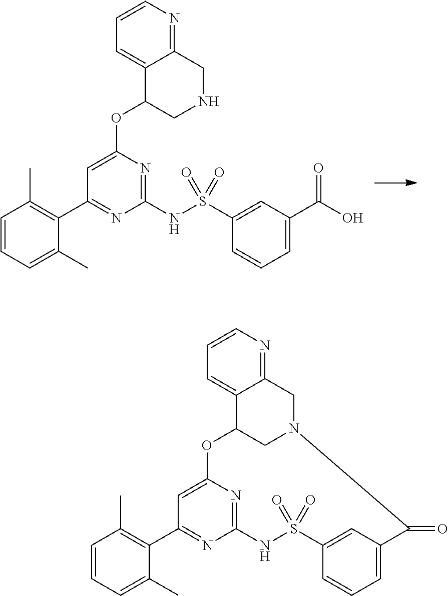 [SMILES: Cc1cccc(C)c1-c1cc(OC2CNCc3ncccc32)nc(NS(=O)(=O)c2cccc(C(=O)O)c2)n1 ; Cc1cccc(C)c1-c1cc2nc(n1)NS(=O)(=O)c1cccc(c1)C(=O)N1Cc3ncccc3C(C1)O2]

To a solution of 3-[[4-(2,6-dimethylphenyl)-6-(5,6,7,8-tetrahydro-1,7-naphthyridin-5-yloxy)pyrimidin-2-yl]sulfamoyl]benzoic acid (hydrochloride salt) (37.6 mg, 0.06619 mmol) in NMP (0.5 mL) was added HATU (35 mg, 0.09205 mmol) followed by DiPEA (47 μL, 0.2698 mmol) and the reaction mixture stirred at room temperature for 1 hour. The reaction mixture was diluted with MeOH, filtered and purification by HPLC (1-99% ACN in water (HCl modifier)) gave 12-(2,6-dimethylphenyl)-15-oxa-8λ⁶-thia-1,9,11,21,25-pentaazapentacyclo[14.7.1.13,7.110,14.017,22]hexacosa-3,5,7(26),10(25),11,13,17,19,21-nonaene-2,8,8-trione (hydrochloride salt) (15 mg, 44%) as an off white solid. ESI-MS m/z calc. 513.1471, found 514.2 (M+1)⁺; Retention time: 1.19 minutes, LC method A.

### Example 98: Preparation of Compound 199 and Compound 200

### Step 1: tert-Butyl 8-hydroxy-1-methyl-2,3,5,7,8,8a-hexahydro-1,6-naphthyridine-6-carboxylate, and tert-butyl 8-hydroxy-1-methyl-2,4a,5,7,8,8a-hexahydro-1,6-naphthyridine-6-carboxylate

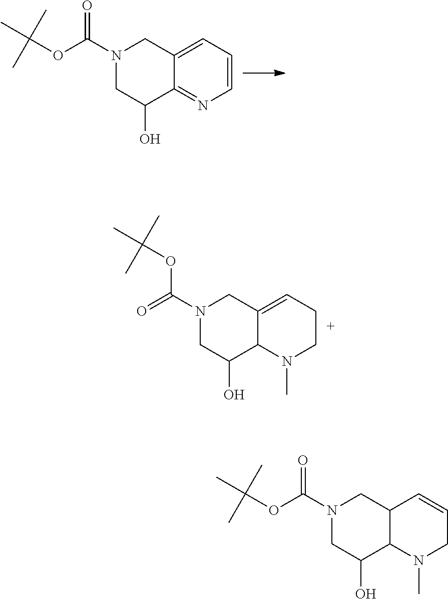 [SMILES: CC(C)(C)OC(=O)N1Cc2cccnc2C(O)C1 ; CN1CCC=C2CN(C(=O)OC(C)(C)C)CC(O)C21 ; CN1CC=CC2CN(C(=O)OC(C)(C)C)CC(O)C21]

tert-Butyl 8-hydroxy-7,8-dihydro-5H-1,6-naphthyridine-6-carboxylate (Iodomethane (1)) (5.3 g, 13.512 mmol) was dissolved in MeOH (50 mL) and the mixture was cooled to −20° C. Sodium borohydride (1.5336 g, 1.6229 mL, 40.536 mmol) was added in small portions periodically over 20 min. The mixture was stirred at this temperature for 1 hour before saturated Sodium bicarbonate was added (20 mL). The mixture was partitioned between DCM (100 mL) and water (60 mL). Layers were separated. The aqueous layer was extracted one more time (50 mL DCM). The combined organics was washed with brine, dried over anhydrous sodium sulfate, filtered and concentrated. tert-Butyl 8-hydroxy-1-methyl-2,3,5,7,8,8a-hexahydro-1,6-naphthyridine-6-carboxylate (3.5 g, 96.52%) was isolated as a mixture of isomers. ESI-MS m/z calc. 268.1787, found 269.6 (M+1)⁺; Retention time: 1.93 minutes, LC method W.

### Step 2: tert-Butyl 8-hydroxy-1-methyl-2,3,4,4a,5,7,8,8a-octahydro-1,6-naphthyridine-6-carboxylate

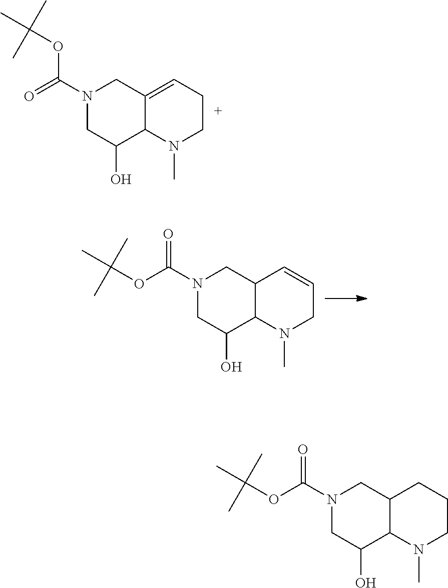 [SMILES: CN1CCC=C2CN(C(=O)OC(C)(C)C)CC(O)C21 ; CN1CC=CC2CN(C(=O)OC(C)(C)C)CC(O)C21 ; CN1CCCC2CN(C(=O)OC(C)(C)C)CC(O)C21]

tert-Butyl 8-hydroxy-1-methyl-2,3,5,7,8,8a-hexahydro-1,6-naphthyridine-6-carboxylate (3.5 g, 12.390 mmol) was dissolved in a solvent mixture of Ethyl acetate and MeOH (40 ml/10 mL). Platinum oxide hydrate (55 mg, 0.2199 mmol) was added to a vial and mixed with 0.5 mL water and transferred to the reaction flask by pipette. The mixture was vacuumed and flushed with nitrogen (3 times). It was vacuumed one more time before a hydrogen balloon was placed and the mixture was stirred at this pressure for 15 hours (balloons was refilled twice during the course of reaction). The mixture was vacuumed and flushed with nitrogen and filtered through a pad of Celite, washed with MeOH (3×30 mL). The filtrate was concentrated. The residue was dissolved in acetonitrile/water (20 mL/20 mL), lyophilized to afford tert-butyl 8-hydroxy-1-methyl-2,3,4,4a,5,7,8,8a-octahydro-1,6-naphthyridine-6-carboxylate (3.2 g, 79%) as a light brown solid. ESI-MS m/z calc. 270.1943, found 271.5 (M+1)⁺; Retention time: 1.27 minutes. ¹H NMR (250 MHz, DMSO (d6)) δ 5.02 (s, 1H), 3.76 (s, 1H), 3.55-3.05 (m, 6H), 2.91 (s, 1H), 2.36 (s, 3H), 2.22 (d, J=12.7 Hz, 1H), 1.66 (s, 3H), 1.39 (d, J=1.1 Hz, 13H). LC method W.

### Step 3: 3-[[4-(2,6-Dimethylphenyl)-6-[(1-methyl-3,4,4a,5,6,7,8,8a-octahydro-2H-1,6-naphthyridin-8-yl)oxy]pyrimidin-2-yl]sulfamoyl]benzoic acid

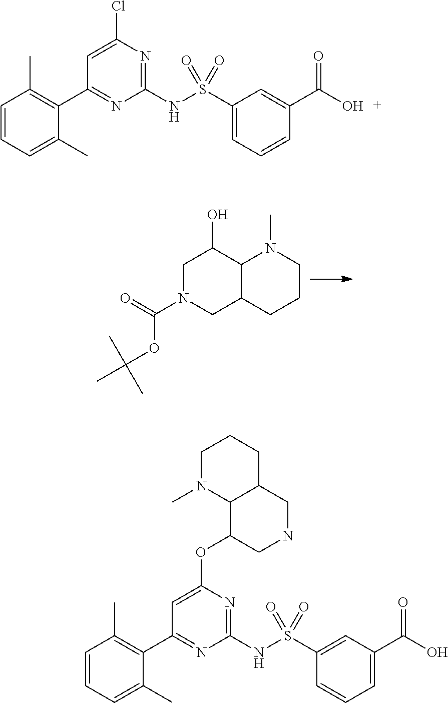 [SMILES: Cc1cccc(C)c1-c1cc(Cl)nc(NS(=O)(=O)c2cccc(C(=O)O)c2)n1 ; CN1CCCC2CN(C(=O)OC(C)(C)C)CC(O)C21 ; Cc1cccc(C)c1-c1cc(OC2CNCC3CCCN(C)C32)nc(NS(=O)(=O)c2cccc(C(=O)O)c2)n1]

To a glass vial was added 3-[[4-chloro-6-(2,6-dimethylphenyl)pyrimidin-2-yl]sulfamoyl]benzoic acid (95 mg, 0.2273 mmol), sodium tert-butoxide (103 mg, 1.072 mmol), and a THE solution of tert-butyl 8-hydroxy-1-methyl-2,3,4,4a,5,7,8,8a-octahydro-1,6-naphthyridine-6-carboxylate (133 mg, 0.4919 mmol). The reaction was stirred at room temperature for 2 h. The reaction mixture was partitioned between ethyl acetate and a 1M HCl solution. The organics were separated, washed with brine, dried over sodium sulfate and evaporated. to afford 3-[[4-[(6-tert-butoxycarbonyl-1-methyl-2,3,4,4a,5,7,8,8a-octahydro-1,6-naphthyridin-8-yl)oxy]-6-(2,6-dimethylphenyl)pyrimidin-2-yl]sulfamoyl]benzoic acid (133 mg, 90%). ESI-MS m/z calc. 651.27264, found 652.38 (M+1)⁺; Retention time: 0.5 minutes (LC method D).

The product was dissolved in 4M HCl in dioxane (2.1 mL of 4 M, 8.400 mmol) and stirred for 30 min. The reaction mixture was evaporated and the crude material 3-[[4-(2,6-dimethylphenyl)-6-[(1-methyl-3,4,4a,5,6,7,8,8a-octahydro-2H-1,6-naphthyridin-8-yl)oxy]pyrimidin-2-yl]sulfamoyl]benzoic acid (hydrochloride salt) (136 mg, 102%) ESI-MS m/z calc. 551.2202, found 552.38 (M+1)⁺; Retention time: 0.32 minutes, LC method D.

### Step 4: 12-(2,6-dimethylphenyl)-18-methyl-15-oxa-8λ⁶-thia-1,9,11,18,25-pentaazapentacyclo[14.7.1.13,7.110,14.017,22]hexacosa-3,5,7(26),10(25),11,13-hexaene-2,8,8-trione

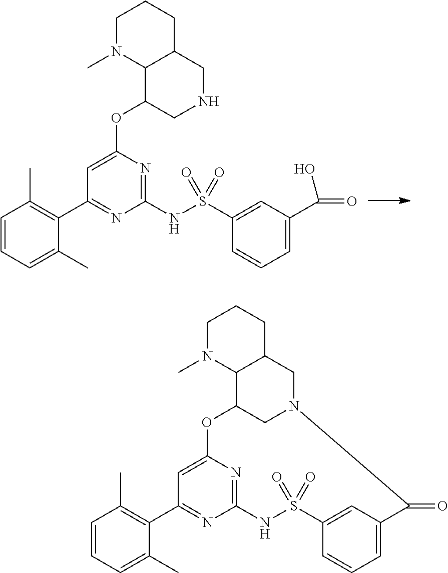

To a round bottom flask was added 3-[[4-(2,6-dimethylphenyl)-6-[(1-methyl-3,4,4a,5,6,7,8,8a-octahydro-2H-1,6-naphthyridin-8-yl)oxy]pyrimidin-2-yl]sulfamoyl]benzoic acid (Dihydrochloride salt) (136 mg, 0.2177 mmol). To the reaction solution was added [dimethylamino(triazolo[4,5-b]pyridin-3-yloxy)methylene]-dimethyl-ammonium (Phosphorus Hexafluoride Ion) (100 mg, 0.2630 mmol) and DIEA (110 μL, 0.6315 mmol). The reaction was allowed to stir at room temp for 1 hour. The reaction solution was filtered and injected directly onto a Reverse-phase Reverse phase HPLC column using a gradient of 1% MeCN in water to 70% MeCN over 15 min to afford purified fractions of 12-(2,6-dimethylphenyl)-18-methyl-15-oxa-8λ⁶-thia-1,9,11,18,25-pentaazapentacyclo[14.7.1.13,7.110,14.017,22]hexacosa-3,5,7(26),10(25),11,13-hexaene-2,8,8-trione (22 mg, 19%) ESI-MS m/z calc. 533.20966, found 534.4 (M+1)⁺; Retention time: 2.25 minutes (LC method A).

### Step 5: 12-(2,6-dimethylphenyl)-18-methyl-15-oxa-8λ⁶-thia-1,9,11,18,25-pentaazapentacyclo[14.7.1.13,7.110,14.017,22]hexacosa-3,5,7(26),10(25),11,13-hexaene-2,8,8-trione, SFC peak 1 (Compound 199), and 12-(2,6-dimethylphenyl)-18-methyl-15-oxa-8λ⁶-thia-1,9,11,18,25-pentaazapentacyclo[14.7.1.13,7.110,14.017,22]hexacosa-3,5,7(26),10(25),11,13-hexaene-2,8,8-trione, SFC peak 2 (Compound 200)

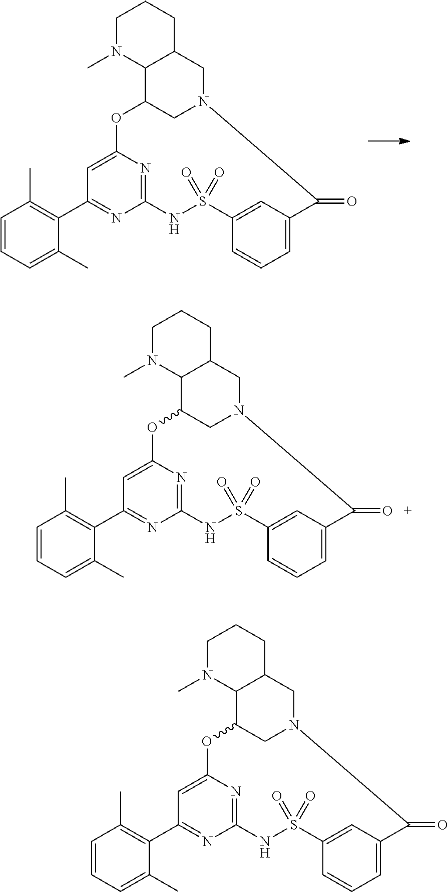

12-(2,6-Dimethylphenyl)-18-methyl-15-oxa-8λ⁶-thia-1,9,11,18,25-pentaazapentacyclo[14.7.1.13,7.110,14.017,22]hexacosa-3,5,7(26),10(25),11,13-hexaene-2,8,8-trione (34 mg, 0.06371 mmol) was dissolved in 1 mL of DMSO and purified by chiral preparative SFC using an AS column, resulting in the separation of two enantiomers: SFC peak 1, 12-(2,6-dimethylphenyl)-18-methyl-15-oxa-8λ⁶-thia-1,9,11,18,25-pentaazapentacyclo[14.7.1.13,7.110,14.017,22]hexacosa-3,5,7(26),10(25),11,13-hexaene-2,8,8-trione (11 mg, 64%) ¹H NMR (400 MHz, DMSO-d₆) δ 11.17 (s, 1H), 8.49 (s, 1H), 7.93 (d, J=7.6 Hz, 1H), 7.70 (dt, J=15.1, 7.5 Hz, 2H), 7.27 (t, J=7.6 Hz, 1H), 7.14 (d, J=7.6 Hz, 2H), 6.62 (s, 1H), 5.47 (dd, J=9.8, 5.7 Hz, 1H), 4.23 (dd, J=13.6, 4.5 Hz, 1H), 4.07 (d, J=9.6 Hz, 1H), 3.83-3.67 (m, 3H), 3.20 (d, J=11.3 Hz, 1H), 2.98 (d, J=4.3 Hz, 3H), 2.77-2.63 (m, 1H), 2.54 (s, 4H), 2.07 (s, 7H), 1.83 (d, J=13.1 Hz, 1H), 1.71 (d, J=13.5 Hz, 2H). ESI-MS m/z calc. 533.20966, found 534.34 (M+1)⁺; Retention time: 0.83 minutes (LC method A); and SFC peak 2, 12-(2,6-dimethylphenyl)-18-methyl-15-oxa-8λ⁶-thia-1,9,11,18,25-pentaazapentacyclo[14.7.1.13,7.110,14.017,22]hexacosa-3,5,7(26),10(25),11,13-hexaene-2,8,8-trione (13 mg, 76%), ESI-MS m/z calc. 533.20966, found 534.37 (M+1)⁺; Retention time: 0.83 minutes, LC method A.

### Example 99: Preparation of Compound 201

### Step 1: tert-Butyl 5-hydroxy-1-methyl-2,3,4,4a,5,6,8,8a-octahydro-1,7-naphthyridine-7-carboxylate, diastereomer 1 and 2

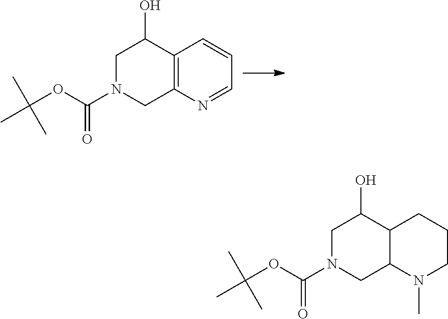 [SMILES: CC(C)(C)OC(=O)N1Cc2ncccc2C(O)C1 ; CN1CCCC2C(O)CN(C(=O)OC(C)(C)C)CC21]

To a solution of tert-butyl 5-hydroxy-6,8-dihydro-5H-1,7-naphthyridine-7-carboxylate (3.22 g, 8.36 mmol) in Methanol (35 mL) and ethyl acetate (10 mL) was added platinum oxide monohydrate (70 mg, 0.31 mmol). The reaction was set in a Parr shaker at 55 psi of hydrogen for 2 hours, at which time the mixture was filtered and concentrated. The crude residue was purified by preparative HPLC (column: Varian C₁₈ 10 um 5×30 cm; flow rate: 60 mL/min.; mobile phase A: water+0.1% TFA; mobile phase B: acetonitrile+0.1% TFA; method:0-45% B in 60 minutes). The combined fractions were basified by 2 N sodium hydroxide, then acetonitrile was removed. The product was extracted with dichloromethane, the organic layer was washed with brine, dried over sodium sulfate and concentrated to give two isomers of tert-butyl 5-hydroxy-1-methyl-2,3,4,4a,5,6,8,8a-octahydro-1,7-naphthyridine-7-carboxylate as yellow Gels: Diastereomer 1 (purified a second time, 502 mg) ESI-MS m/z calc. 270.19, found 271.5 (M+1)⁺; Retention time: 1.71 minutes (LC method T). ¹H NMR (250 MHz, DMSO-d₆) δ 4.78 (d, J=5.3 Hz, 1H), 4.26-3.84 (m, 2H), 3.74 (tt, J=10.1, 5.2 Hz, 1H), 2.72 (d, J=14.5 Hz, 2H), 2.42-2.26 (m, 1H), 2.15 (s, 3H), 1.97 (d, J=18.5 Hz, 3H), 1.76-1.52 (m, 1H), 1.39 (s, 10H), 1.27-1.09 (m, 1H), and diastereomer 2 (1.62 g), ESI-MS m/z calc. 270.19, found 271.5 (M+1)⁺; Retention time: 1.74 minutes (LC method T), ¹H NMR (250 MHz, DMSO-d6) δ 5.02 (d, J=6.6 Hz, 1H), 3.53 (d, J=5.5 Hz, 1H), 3.42 (s, 2H), 3.01 (s, 1H), 2.39 (s, 1H), 2.35-2.20 (m, 4H), 2.14 (d, J=5.5 Hz, 1H), 2.00-1.70 (m, 2H), 1.66-1.43 (m, 2H), 1.39 (s, 11H).

### Step 2: 3-[[4-(2,6-Dimethylphenyl)-6-[(1-methyl-3,4,4a,5,6,7,8,8a-octahydro-2H-1,7-naphthyridin-5-yl)oxy]pyrimidin-2-yl]sulfamoyl]benzoic acid, diastereomer 1

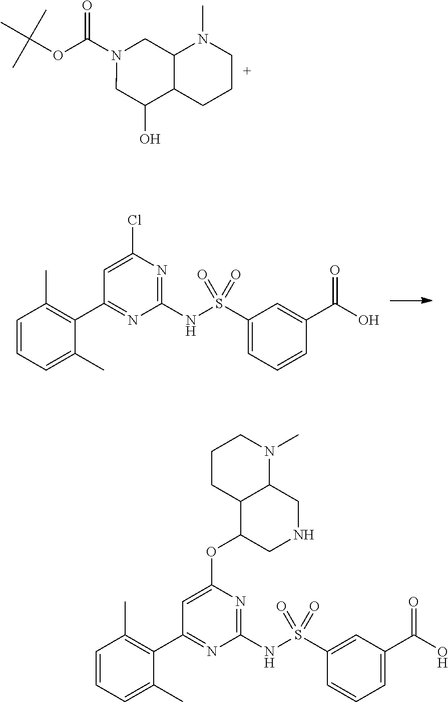 [SMILES: CN1CCCC2C(O)CN(C(=O)OC(C)(C)C)CC21 ; Cc1cccc(C)c1-c1cc(Cl)nc(NS(=O)(=O)c2cccc(C(=O)O)c2)n1 ; Cc1cccc(C)c1-c1cc(OC2CNCC3C2CCCN3C)nc(NS(=O)(=O)c2cccc(C(=O)O)c2)n1]

To a round bottom flask was added 3-[[4-chloro-6-(2,6-dimethylphenyl)pyrimidin-2-yl]sulfamoyl]benzoic acid (110 mg, 0.2632 mmol), tert-butyl 5-hydroxy-1-methyl-2,3,4,4a,5,6,8,8a-octahydro-1,7-naphthyridine-7-carboxylate (trifluoroacetate salt) (diastereomer 1,150 mg, 0.3902 mmol), anhydrous THF (2 mL), and sodium tert-butoxide (100 mg, 1.041 mmol). The reaction solution was allowed to stir at 23° C. for 1 h. To the reaction solution was added NaH (60 mg of 30% w/w, 0.7501 mmol) and the reaction solution was allowed to stir at 50° C. overnight. To the reaction solution was added HCl (3 mL of 4 M, 12.00 mmol) and the solution was stirred 1 hour at room temperature. The reaction solution was filtered and purified by reverse phase HPLC using a gradient of 1% MeCN in water to 70% MeCN over 15 min to afford purified fractions of 3-[[4-(2,6-dimethylphenyl)-6-[(1-methyl-3,4,4a,5,6,7,8,8a-octahydro-2H-1,7-naphthyridin-5-yl)oxy]pyrimidin-2-yl]sulfamoyl]benzoic acid, diastereomer 1 (45 mg, 31%) ESI-MS m/z calc. 551.2202, found 552.38 (M+1)⁺; Retention time: 0.3 minutes, LC method D.

### Step 3: 12-(2,6-Dimethylphenyl)-21-methyl-15-oxa-8λ⁶-thia-1,9,11,21,25-pentaazapentacyclo[14.7.1.13,7.110,14.017,22]hexacosa-3,5,7(26),10(25),11,13-hexaene-2,8,8-trione, diastereomer 1 (Compound 201)

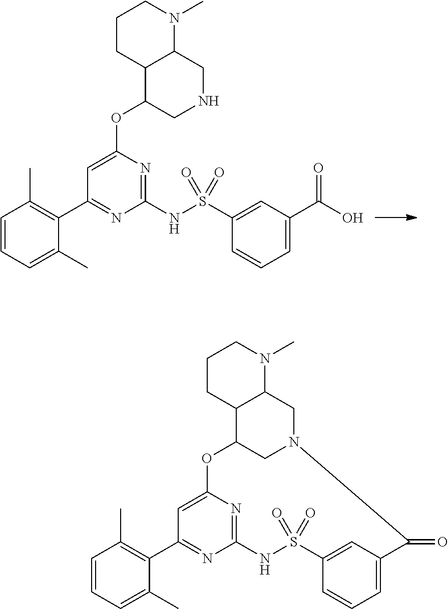 [SMILES: Cc1cccc(C)c1-c1cc(OC2CNCC3C2CCCN3C)nc(NS(=O)(=O)c2cccc(C(=O)O)c2)n1 ; Cc1cccc(C)c1-c1cc2nc(n1)NS(=O)(=O)c1cccc(c1)C(=O)N1CC(O2)C2CCCN(C)C2C1]

To a nitrogen sparged round bottom flask was added 3-[[4-(2,6-dimethylphenyl)-6-[(1-methyl-3,4,4a,5,6,7,8,8a-octahydro-2H-1,7-naphthyridin-5-yl)oxy]pyrimidin-2-yl]sulfamoyl]benzoic acid, diastereomer 1 (34 mg, 0.06163 mmol), HATU (28 mg, 0.07364 mmol), DMF (3 mL), and DIEA (24 mg, 0.1857 mmol). The reaction solution was stirred at room temperature for 1 hr. The reaction mixture was concentrated in vacuo and the crude residue was purified by reverse phase HPLC using a gradient of 1% MeCN in water to 99% MeCN over 15 min to afford 12-(2,6-dimethylphenyl)-21-methyl-15-oxa-8λ⁶-thia-1,9,11,21,25-pentaazapentacyclo[14.7.1.13,7.110,14.017,22]hexacosa-3,5,7(26),10(25),11,13-hexaene-2,8,8-trione, diastereomer 1 (18 mg, 54%) ESI-MS m/z calc. 533.20966, found 534.34 (M+1)⁺; Retention time: 0.97 minutes, LC method A.

### Example 100: Preparation of Compound 202

### Step 1: 3-[[4-(2,6-Dimethylphenyl)-6-[(1-methyl-3,4,4a,5,6,7,8,8a-octahydro-2H-1,7-naphthyridin-5-yl)oxy]pyrimidin-2-yl]sulfamoyl]benzoic acid, diastereomer 2

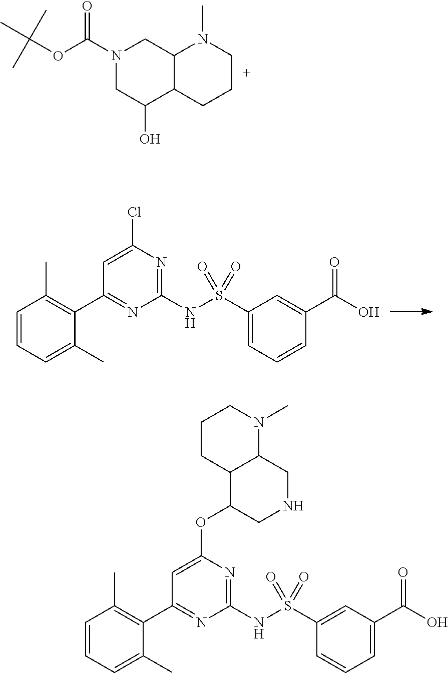

To a glass vial containing tert-butyl 5-hydroxy-1-methyl-2,3,4,4a,5,6,8,8a-octahydro-1,7-naphthyridine-7-carboxylate (trifluoroacetate salt), diastereomer 2 (160 mg, 0.4162 mmol) was added 3-[[4-chloro-6-(2,6-dimethylphenyl)pyrimidin-2-yl]sulfamoyl]benzoic acid (145 mg, 0.3470 mmol), sodium tert-butoxide (83 mg, 0.8637 mmol) and anhydrous THE (2 mL). The reaction was allowed to stir at room temp for 1 hour. LCMS found 1:1 mixture of product M+H=552 plus SM., and To the reaction solution was added NaH (28 mg of 60% w/w, 0.7001 mmol), and the mixture was stirred another 1 hour at room temperature, then 1 hour at 50° C. The reaction solution was quenched by addition of 1 mL MeOH. The reaction mixture was filtered and purified by reverse phase HPLC using a gradient of 1% MeCN in water to 70% MeCN over 15 min to afford 3-[[4-(2,6-dimethylphenyl)-6-[(1-methyl-3,4,4a,5,6,7,8,8a-octahydro-2H-1,7-naphthyridin-5-yl)oxy]pyrimidin-2-yl]sulfamoyl]benzoic acid, diastereomer 2 (60 mg, 31%) ESI-MS m/z calc. 551.2202, found 552.34 (M+1)⁺; Retention time: 0.9 minutes, LC method A.

### Step 2: 12-(2,6-Dimethylphenyl)-21-methyl-15-oxa-8λ⁶-thia-1,9,11,21,25-pentaazapentacyclo[14.7.1.13,7.110,14.017,22]hexacosa-3,5,7(26),10(25),11,13-hexaene-2,8,8-trione, diastereomer 2 (Compound 202)

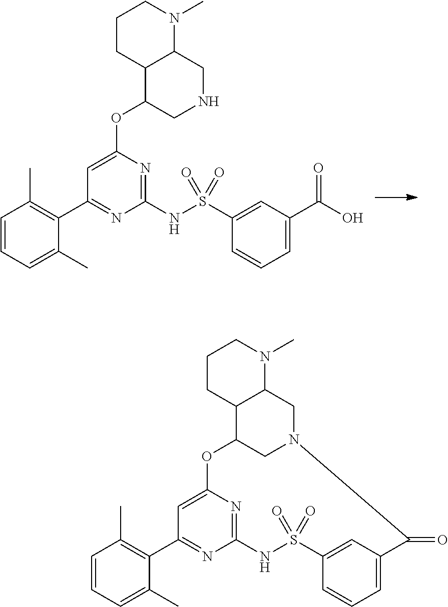 [SMILES: Cc1cccc(C)c1-c1cc(OC2CNCC3C2CCCN3C)nc(NS(=O)(=O)c2cccc(C(=O)O)c2)n1 ; Cc1cccc(C)c1-c1cc2nc(n1)NS(=O)(=O)c1cccc(c1)C(=O)N1CC(O2)C2CCCN(C)C2C1]

To a nitrogen sparged round bottom flask was added 3-[[4-(2,6-dimethylphenyl)-6-[(1-methyl-3,4,4a,5,6,7,8,8a-octahydro-2H-1,7-naphthyridin-5-yl)oxy]pyrimidin-2-yl]sulfamoyl]benzoic acid, diastereomer 2 (60 mg, 0.1088 mmol), HATU (51 mg, 0.1341 mmol), DMF (4 mL) and DIEA (60 μL, 0.3445 mmol). The reaction solution was stirred at room temperature for 40 min. The reaction mixture was concentrated in vacuo and the crude residue purified by reverse phase HPLC using a gradient of 1% MeCN in water to 99% MeCN over 15 min to afford of 12-(2,6-dimethylphenyl)-21-methyl-15-oxa-8λ⁶-thia-1,9,11,21,25-pentaazapentacyclo[14.7.1.13,7.110,14.017,22]hexacosa-3,5,7(26),10(25),11,13-hexaene-2,8,8-trione (6 mg, 10%) ESI-MS m/z calc. 533.20966, found 534.34 (M+1)⁺; Retention time: 0.67 minutes, LC method A.

### Example 101: Preparation of Compound 203 and Compound 204

### Step 1: 12-(2,6-dimethylphenyl)-21-methyl-15-oxa-8λ⁶-thia-1,9,11,21,25-pentaazapentacyclo[14.7.1.13,7.110,14.017,22]hexacosa-3,5,7(26),10(25),11,13-hexaene-2,8,8-trione, diastereomer 3 (Compound 203), and 12-(2,6-dimethylphenyl)-21-methyl-15-oxa-8λ⁶-thia-1,9,11,21,25-pentaazapentacyclo[14.7.1.13,7.110,14.017,22]hexacosa-3,5,7(26),10(25),11,13-hexaene-2,8,8-trione, diastereomer 4 (Compound 204)

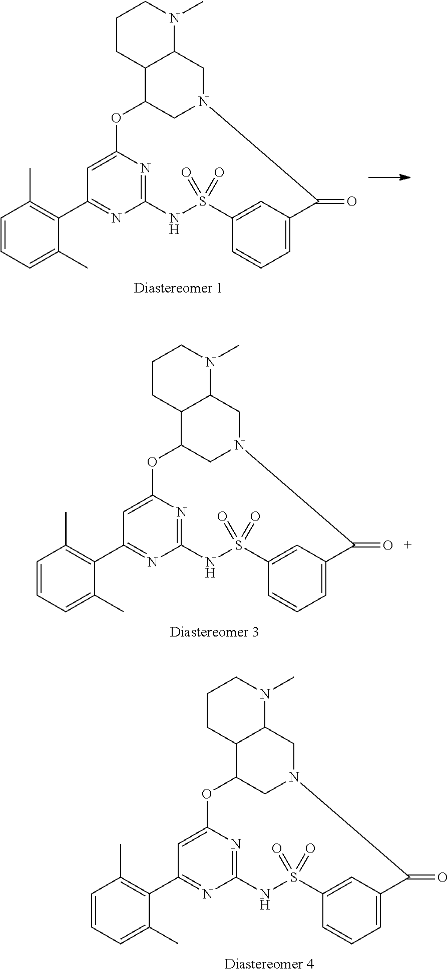 [SMILES: Cc1cccc(C)c1-c1cc2nc(n1)NS(=O)(=O)c1cccc(c1)C(=O)N1CC(O2)C2CCCN(C)C2C1 ; Cc1cccc(C)c1-c1cc2nc(n1)NS(=O)(=O)c1cccc(c1)C(=O)N1CC(O2)C2CCCN(C)C2C1 ; Cc1cccc(C)c1-c1cc2nc(n1)NS(=O)(=O)c1cccc(c1)C(=O)N1CC(O2)C2CCCN(C)C2C1]

12-(2,6-Dimethylphenyl)-21-methyl-15-oxa-8λ⁶-thia-1,9,11,21,25-pentaazapentacyclo[14.7.1.13,7.110,14.017,22]hexacosa-3,5,7(26),10(25),11,13-hexaene-2,8,8-trione, diastereomer 1, (17 mg, 0.03186 mmol) was dissolved in DMF (1.5 mL) and purified by chiral preparative SFC using an AS column to give two isomers: 12-(2,6-Dimethylphenyl)-21-methyl-15-oxa-8λ⁶-thia-1,9,11,21,25-pentaazapentacyclo[14.7.1.13,7.110,14.017,22]hexacosa-3,5,7(26),10(25),11,13-hexaene-2,8,8-trione, diastereomer 3 (4.3 mg, 50%) ESI-MS m/z calc. 533.20966, found 534.37 (M+1)⁺; Retention time: 0.98 minutes (LC method A); and 12-(2,6-dimethylphenyl)-21-methyl-15-oxa-8λ⁶-thia-1,9,11,21,25-pentaazapentacyclo[14.7.1.13,7.110,14.017,22]hexacosa-3,5,7(26),10(25),11,13-hexaene-2,8,8-trione, diastereomer 4 (4.7 mg, 55%), ESI-MS m/z calc. 533.20966, found 534.37 (M+1)⁺; Retention time: 0.98 minutes, LC method A.

### Example 102: Preparation of (3R,7R)-19-(2,6-Dimethylphenyl)-5-{spiro[3.5]nonan-2-yl}-8-{2-[1-(trifluoromethyl)cyclopropyl]ethyl}-2-oxa-15λ⁶-thia-5,8,16,18,21-pentaazatetracyclo[15.3.1.110,14.03,7]docosa-1(20),10(22),11,13,17(21),18-hexaene-9,15,15-trione

### Step 1: 3-[[4-[(3R,4R)-4-Amino-1-tert-butoxycarbonyl-pyrrolidin-3-yl]oxy-6-(2,6-dimethylphenyl)pyrimidin-2-yl]sulfamoyl]benzoic acid

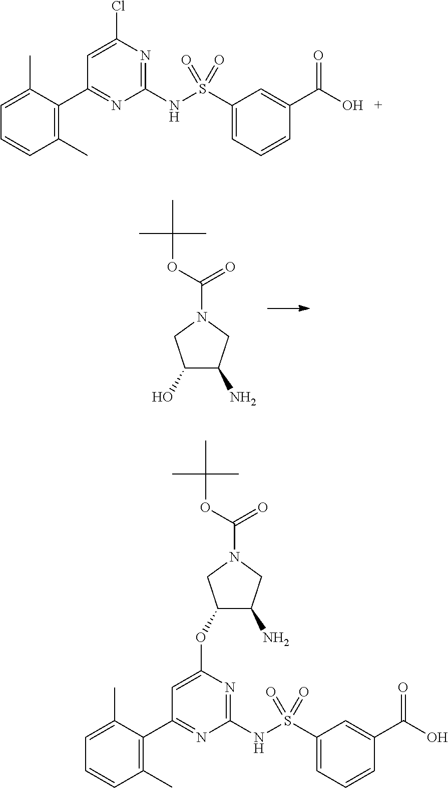

A solution of 3-[[4-chloro-6-(2,6-dimethylphenyl)pyrimidin-2-yl]sulfamoyl]benzoic acid (10.6 g, 25.37 mmol), tert-butyl (3R,4R)-3-amino-4-hydroxy-pyrrolidine-1-carboxylate (5.2 g, 25.71 mmol), and sodium t-butoxide (7.3 g, 75.96 mmol) in THE (0.13 L) was stirred for 18 hours. The reaction was acidified with 1 M citric acid, diluted with water, and extracted with ethyl acetate. The combined extracts were washed with brine, dried over sodium sulfate, and evaporated under vacuum to give a tan oil. The oil was stirred with diethyl ether to give a colorless solid. The solid was filtered, washed with diethyl ether, and dried under vacuum to give 3-[[4-[(3R,4R)-4-amino-1-tert-butoxycarbonyl-pyrrolidin-3-yl]oxy-6-(2,6-dimethylphenyl)pyrimidin-2-yl]sulfamoyl]benzoic acid (15.2 g, 103%) ESI-MS m/z calc. 583.2101, found 584.3 (M+1)⁺; Retention time: 0.49 minutes as a colorless solid, LC method D.

### Step 2: 3-[[4-[(3R,4R)-1-tert-Butoxycarbonyl-4-[2-[1-(trifluoromethyl)cyclopropyl]ethylamino]pyrrolidin-3-yl]oxy-6-(2,6-dimethylphenyl)pyrimidin-2-yl]sulfamoyl]benzoic acid

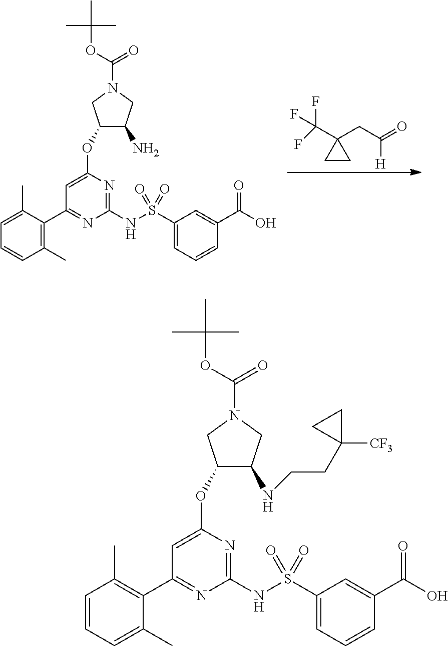 [SMILES: Cc1cccc(C)c1-c1cc(O[C@@H]2CN(C(=O)OC(C)(C)C)C[C@H]2N)nc(NS(=O)(=O)c2cccc(C(=O)O)c2)n1 ; O=CCC1(C(F)(F)F)CC1 ; Cc1cccc(C)c1-c1cc(O[C@@H]2CN(C(=O)OC(C)(C)C)C[C@H]2NCCC2(C(F)(F)F)CC2)nc(NS(=O)(=O)c2cccc(C(=O)O)c2)n1]

A solution of 3-[[4-[(3R,4R)-4-amino-1-tert-butoxycarbonyl-pyrrolidin-3-yl]oxy-6-(2,6-dimethylphenyl)pyrimidin-2-yl]sulfamoyl]benzoic acid (291.8 mg, 0.5 mmol), 2-[1-(trifluoromethyl)cyclopropyl]acetaldehyde (approximately 83.66 mg, 0.5500 mmol), and sodium triacetoxyborohydride (approximately 211.9 mg, 1.000 mmol) in dichloromethane (2.500 mL) was stirred for 18 hours. More 2-[1-(trifluoromethyl)cyclopropyl]acetaldehyde (approximately 83.66 mg, 0.5500 mmol) and sodium triacetoxyborohydride (approximately 211.9 mg, 1.000 mmol) were again added, and the reactions were stirred for 22 hours. The solvent was evaporated, and the residue was diluted with water, acidified with 1 M citric acid, and extracted with ethyl acetate. The residue was purified by silica gel column chromatography with 0-8% methanol in dichloromethane to give 3-[[4-[(3R,4R)-1-tert-butoxycarbonyl-4-[2-[1-(trifluoromethyl)cyclopropyl]ethylamino]pyrrolidin-3-yl]oxy-6-(2,6-dimethylphenyl)pyrimidin-2-yl]sulfamoyl]benzoic acid (0.19 g, 53%). ESI-MS m/z calc. 719.2601, found 720.3 (M+1)⁺; Retention time: 0.57 minutes; LC method D.

### Step 3: tert-Butyl (3R,7R)-19-(2,6-dimethylphenyl)-9,15,15-trioxo-8-{2-[1-(trifluoromethyl)cyclopropyl]ethyl}-2-oxa-15λ⁶-thia-5,8,16,18,21-pentaazatetracyclo[15.3.1.110,14.03,7]docosa-1(20),10(22),11,13,17(21),18-hexaene-5-carboxylate

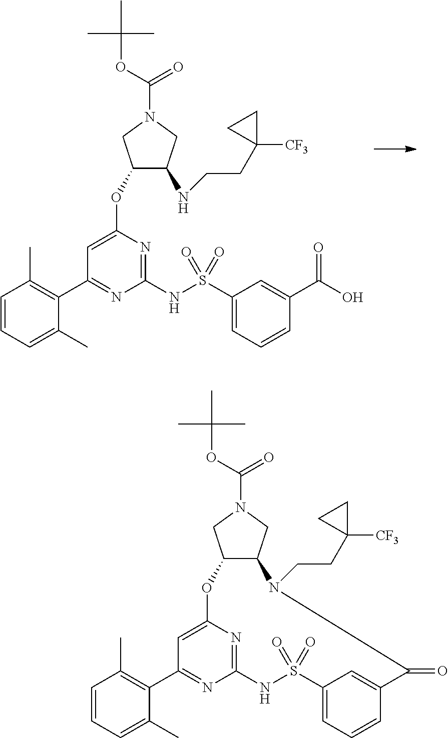 [SMILES: Cc1cccc(C)c1-c1cc(O[C@@H]2CN(C(=O)OC(C)(C)C)C[C@H]2NCCC2(C(F)(F)F)CC2)nc(NS(=O)(=O)c2cccc(C(=O)O)c2)n1 ; Cc1cccc(C)c1-c1cc2nc(n1)NS(=O)(=O)c1cccc(c1)C(=O)N(CCC1(C(F)(F)F)CC1)[C@@H]1CN(C(=O)OC(C)(C)C)C[C@H]1O2]

A solution of 3-[[4-[(3R,4R)-1-tert-butoxycarbonyl-4-[2-[1-(trifluoromethyl)cyclopropyl]ethylamino]pyrrolidin-3-yl]oxy-6-(2,6-dimethylphenyl)pyrimidin-2-yl]sulfamoyl]benzoic acid (0.19 g, 0.2640 mmol), [[(E)-(1-cyano-2-ethoxy-2-oxo-ethylidene)amino]oxy-tetrahydropyran-4-yl-methylene]-dimethyl-ammonium (Phosphorus Hexafluoride Ion) (0.17 g, 0.3979 mmol), and DIEA (0.14 mL, 0.8038 mmol) in DMF (25 mL) was stirred for three days. The reaction was acidified with 1 M citric acid, diluted with water, and extracted with ethyl acetate. The combined extracts were washed with brine, dried over sodium sulfate, and evaporated under vacuum. The residue was purified by silica gel column chromatography with 0-8% methanol in dichloromethane to give tert-butyl (3R,7R)-19-(2,6-dimethylphenyl)-9,15,15-trioxo-8-{2-[1-(trifluoromethyl)cyclopropyl]ethyl}-2-oxa-15λ⁶-thia-5,8,16,18,21-pentaazatetracyclo[15.3.1.110,14.03,7]docosa-1(20),10(22),11,13,17(21),18-hexaene-5-carboxylate (0.17 g, 92%) as an orange solid. ESI-MS m/z calc. 701.2495, found 702.3 (M+1)⁺; Retention time: 0.75 minutes, LC method D.

### Step 4: (3R,7R)-19-(2,6-Dimethylphenyl)-8-{2-[1-(trifluoromethyl)cyclopropyl]ethyl}-2-oxa-15λ⁶-thia-5,8,16,18,21-pentaazatetracyclo[15.3.1.110,14.03,7]docosa-1(20),10(22),11,13,17(21),18-hexaene-9,15,15-trione

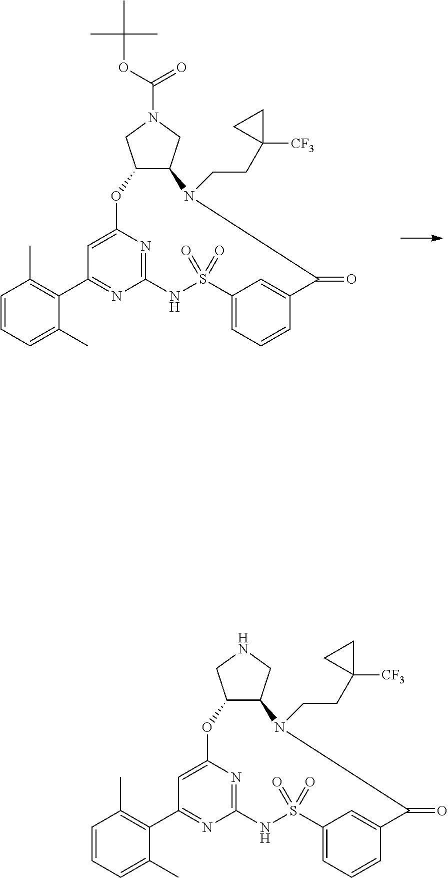 [SMILES: Cc1cccc(C)c1-c1cc2nc(n1)NS(=O)(=O)c1cccc(c1)C(=O)N(CCC1(C(F)(F)F)CC1)[C@@H]1CN(C(=O)OC(C)(C)C)C[C@H]1O2 ; Cc1cccc(C)c1-c1cc2nc(n1)NS(=O)(=O)c1cccc(c1)C(=O)N(CCC1(C(F)(F)F)CC1)[C@@H]1CNC[C@H]1O2]

A solution of tert-butyl (3R,7R)-19-(2,6-dimethylphenyl)-9,15,15-trioxo-8-{2-[1-(trifluoromethyl)cyclopropyl]ethyl}-2-oxa-15λ⁶-thia-5,8,16,18,21-pentaazatetracyclo[15.3.1.110,14.03,7]docosa-1(20),10(22),11,13,17(21),18-hexaene-5-carboxylate (0.17 g, 0.2422 mmol) in HCl (4 mL of 4 M, 16.00 mmol) (in dioxane) was stirred for 30 minutes, and the solvent was removed under vacuum. The solids were triturated with diethyl ether and dried under vacuum to give (3R,7R)-19-(2,6-dimethylphenyl)-8-{2-[1-(trifluoromethyl)cyclopropyl]ethyl}-2-oxa-15λ⁶-thia-5,8,16,18,21-pentaazatetracyclo[15.3.1.110,14.03,7]docosa-1(20),10(22),11,13,17(21),18-hexaene-9,15,15-trione (hydrochloride salt) (0.14 g, 91%) as a colorless solid. ESI-MS m/z calc. 601.1971, found 602.3 (M+1)⁺; Retention time: 0.4 minutes, LC method D.

### Step 5: (3R,7R)-19-(2,6-Dimethylphenyl)-5-{spiro[3.5]nonan-2-yl}-8-{2-[1-(trifluoromethyl)cyclopropyl]ethyl}-2-oxa-15λ⁶-thia-5,8,16,18,21-pentaazatetracyclo[15.3.1.110,14.03,7]docosa-1(20),10(22),11,13,17(21),18-hexaene-9,15,15-trione

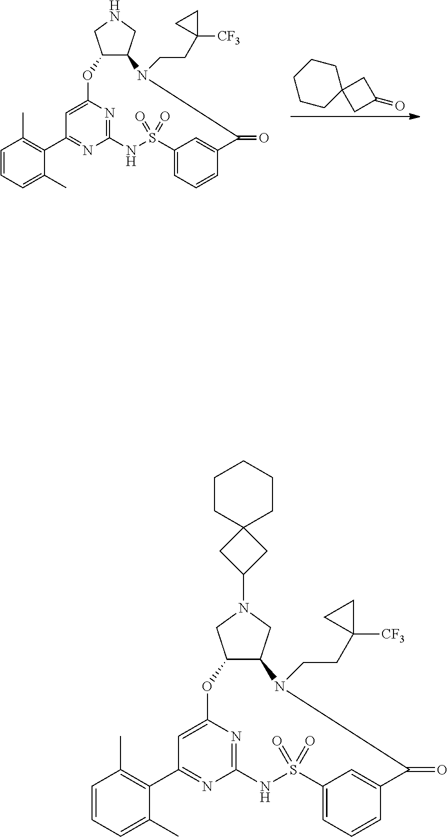 [SMILES: Cc1cccc(C)c1-c1cc2nc(n1)NS(=O)(=O)c1cccc(c1)C(=O)N(CCC1(C(F)(F)F)CC1)[C@@H]1CNC[C@H]1O2 ; O=C1CC2(CCCCC2)C1 ; Cc1cccc(C)c1-c1cc2nc(n1)NS(=O)(=O)c1cccc(c1)C(=O)N(CCC1(C(F)(F)F)CC1)[C@@H]1CN(C3CC4(CCCCC4)C3)C[C@H]1O2]

A solution of (3R,7R)-19-(2,6-dimethylphenyl)-8-{2-[1-(trifluoromethyl)cyclopropyl]ethyl}-2-oxa-15λ⁶-thia-5,8,16,18,21-pentaazatetracyclo[15.3.1.110,14.03,7]docosa-1(20),10(22),11,13,17(21),18-hexaene-9,15,15-trione (hydrochloride salt) (40 mg, 0.06269 mmol), spiro[3.5]nonan-2-one (18 mg, 0.1302 mmol), and sodium triacetoxyborohydride (41 mg, 0.1935 mmol) in dichloromethane (0.3 mL) was stirred for four hours. More spiro[3.5]nonan-2-one (18 mg, 0.1302 mmol) and sodium triacetoxyborohydride (41 mg, 0.1935 mmol) were added, and the reaction was stirred for four days. The reaction was stirred with methanol, and the solvents were removed under vacuum. The residue was purified by reverse-phase HPLC-MS (1%-99% acetonitrile/water (5 mM HCl)) to give (3R,7R)-19-(2,6-dimethylphenyl)-5-{spiro[3.5]nonan-2-yl}-8-{2-[1-(trifluoromethyl)cyclopropyl]ethyl}-2-oxa-15λ⁶-thia-5,8,16,18,21-pentaazatetracyclo[15.3.1.110,14.03,7]docosa-1(20),10(22),11,13,17(21),18-hexaene-9,15,15-trione (hydrochloride salt) (17.8 mg, 37%) as a light yellow solid. ESI-MS m/z calc. 723.30664, found 724.5 (M+1)⁺; Retention time: 1.61 minutes, LC method A.

### Example 103: Preparation of Compound 205

### Step 1: 2-nitro-N-[3-[(2-nitrophenyl)sulfonylamino]propyl]benzenesulfonamide

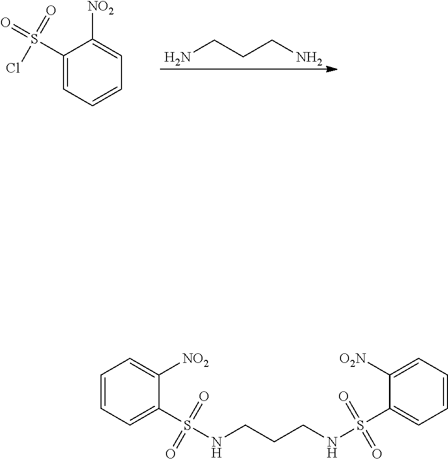 [SMILES: O=[N+]([O-])c1ccccc1S(=O)(=O)Cl ; NCCCN ; O=[N+]([O-])c1ccccc1S(=O)(=O)NCCCNS(=O)(=O)c1ccccc1[N+](=O)[O-]]

Propane-1,3-diamine (2 mL, 23.43 mmol) was added to a solution of potassium carbonate (6.5 g, 47.0 mmol) in water (20 mL). A solution of 2-nitro-benzenesulfonyl chloride (10.7 g, 46.8 mmol) in THE (40 mL) was added to the solution over a period of 1 hour. The reaction was stirred at rt for 2 hours. THE was removed and the residue was poured into ice-water, filtered, washed with cold EtOH and dried under vacuum to get 4-methyl-N-[3-(p-tolylsulfonylamino)propyl]benzenesulfonamide (9.3 g, 85%) as white solid. ¹H NMR (250 MHz, CDCl₃) δ 8.24-8.09 (m, 2H), 7.94-7.68 (m, 6H), 5.60 (t, J=6.5 Hz, 2H), 3.24 (q, J=6.4 Hz, 4H), 1.79 (q, J=6.2 Hz, 2H). ESI-MS m/z calc. 444.0, found 445.4 (M+1)⁺; Retention time: 2.57 minutes (LC method T).

### Step 2: 1,5-bis[(2-Nitrophenyl)sulfonyl]-1,5-diazocan-3-ol

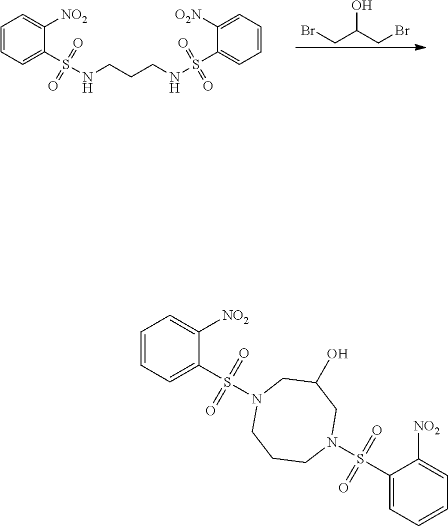 [SMILES: O=[N+]([O-])c1ccccc1S(=O)(=O)NCCCNS(=O)(=O)c1ccccc1[N+](=O)[O-] ; OC(CBr)CBr ; O=[N+]([O-])c1ccccc1S(=O)(=O)N1CCCN(S(=O)(=O)c2ccccc2[N+](=O)[O-])CC(O)C1]

A solution of NaOMe (27 mL, 30% in MeOH, 125 mmol) was added to a solution of 2-nitro-N-[3-[(2-nitrophenyl)sulfonylamino]propyl]benzenesulfonamide (18 g, 38.5 mmol) in anhydrous methanol (500 mL) slowly. The solution was heated to reflux for one hour. The solvent was removed under reduce pressure and the residue was dissolved into ethanol (700 mL). After 1,3-dibromopropan-2-ol (6.8 ml, 60 mmol) was added, the system was heated refluxed for 14 hours. The system was cooled to rt., filtrated to give [3-hydroxy-5-(2-nitrophenyl) sulfonyl-1,5-diazocan-1-yl]-(2-nitrothiopyran-1-yl)methanedione (15.9 g, 83%) as white solid. ESI-MS m/z calc. 500.1, found 501.4 (M+1)⁺; Retention time: 4.54 minutes, LC method S.

### Step 3: tert-Butyl 3-hydroxy-5-(2-nitrophenyl)sulfonyl-1,5-diazocane-1-carboxylate

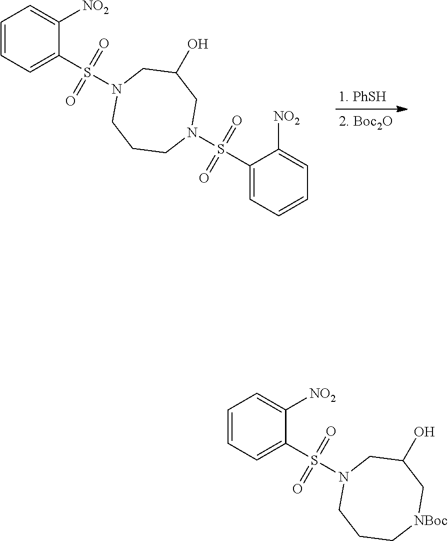 [SMILES: O=[N+]([O-])c1ccccc1S(=O)(=O)N1CCCN(S(=O)(=O)c2ccccc2[N+](=O)[O-])CC(O)C1 ; CC(C)(C)OC(=O)N1CCCN(S(=O)(=O)c2ccccc2[N+](=O)[O-])CC(O)C1]

To a solution of 1,5-bis[(2-nitrophenyl)sulfonyl]-1,5-diazocan-3-ol (17 g, 32.3 mmol), potassium carbonate (7.1 g, 51.4 mmol) in DMF (150 mL) was added thiophenol (4.4 mL, 43 mmol). The system was stirred at rt for 14 hours. The reaction mixture poured into water (120 mL) and washed with EtOAc (3×80 mL). Water of aqueous phase was removed under reduce pressure. To the solution was added Boc anhydride (8.5 g, 38.9 mmol), and TEA (8 mL, 57.4 mmol). The reaction was stirred at rt for 2 hours. The reaction was quenched with saturated aqueous ammonium chloride (50 mL) and extracted with EtOAc (3×70 mL). The combined organic layer was washed with water (3×50 mL), brine, dried over anhydrous sodium sulfate, filtered, and concentrated to afford crude tert-butyl 3-hydroxy-5-(2-nitrophenyl)sulfonyl-1,5-diazocane-1-carboxylate (10 g, 67%) as white solid without further purification. ESI-MS m/z calc. 415.1, found 415.8 (M+1)⁺; Retention time: 2.8 minutes, LC method T.

### Step 4: tert-Butyl 3-hydroxy-1,5-diazocane-1-carboxylate

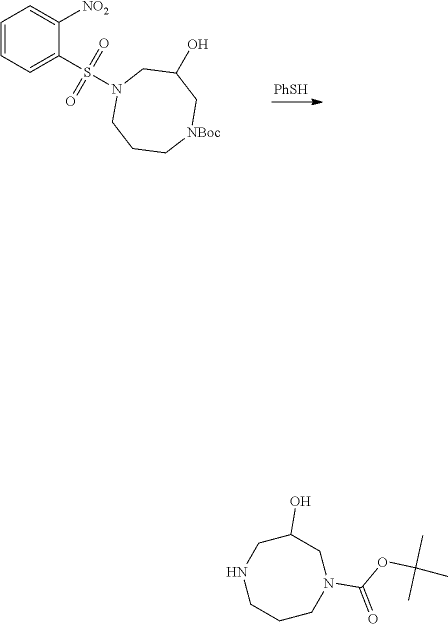 [SMILES: CC(C)(C)OC(=O)N1CCCN(S(=O)(=O)c2ccccc2[N+](=O)[O-])CC(O)C1 ; "PhSH" ; CC(C)(C)OC(=O)N1CCCNCC(O)C1]

To a solution of tert-butyl 3-hydroxy-5-(2-nitrophenyl)sulfonyl-1,5-diazocane-1-carboxylate (6 g, 13 mmol) potassium carbonate (3.5 g, 25.3 mmol) in DMF (50 mL) was added thiophenol (2 mL, 19.6 mmol). The system was stirred at rt for 14 hours. The reaction mixture poured into water (40 mL) and washed with EtOAc (3×20 mL). The solvents of aqueous phase were removed under reduce pressure. The residue was purified by silica gel column chromatography using 0-30% methanol/DCM to afford tert-butyl 3-hydroxy-1,5-diazocane-1-carboxylate (1.3 g, 41%) as a colorless oil. ESI-MS m/z calc. 230.163, found 231.4 (M+1)⁺; Retention time: 1.79 minutes, LC method T.

### Step 5: tert-Butyl 5-[3-[[4-chloro-6-(2,6-dimethylphenyl)pyrimidin-2-yl]sulfamoyl]benzoyl]-3-hydroxy-1,5-diazocane-1-carboxylate

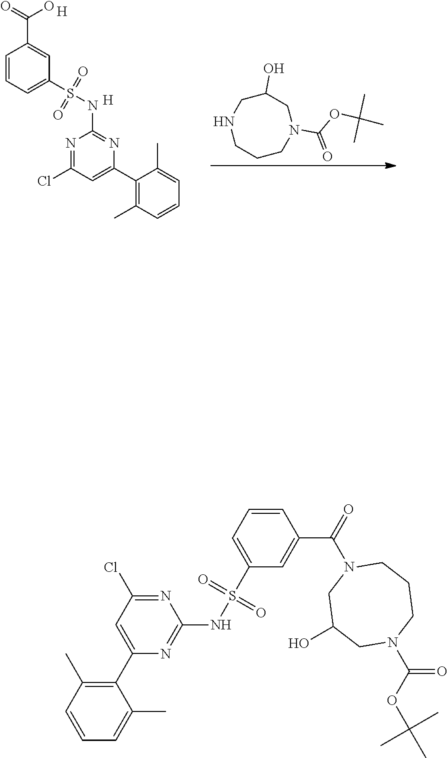

To a solution of 3-[4-chloro-6-(2,6-dimethyl-phenyl)-pyrimidin-2-ylsulfamoyl]-benzoic acid (1.25 g, 4.54 mmol) and tert-butyl 3-hydroxy-1,5-diazocane-1-carboxylate (1.06 g, 4.6 mmol) in DMF (30 mL) and DIEA (8.0 mL, 45.9 mmol) was added HATU (2.33 g, 6.07 mmol) at 0° C. The reaction mixture was stirred for 10 minutes under ice-salt bath. Then reaction was quenched with 10% aqueous citric acid solution (20 mL). The two layers were separated. The aqueous layer was extracted with EtOAc (3×50 mL) and the combined organic layers were washed with brine (50 mL), dried over anhydrous sodium sulfate and concentrated. The residue was purified by silica gel column chromatography using 0-80% hexanes-acetone to furnish tert-butyl 5-[3-[[4-chloro-6-(2,6-dimethylphenyl)pyrimidin-2-yl]sulfamoyl]benzoyl]-3-hydroxy-1,5-diazocane-1-carboxylate (1.25 g, 46%) as a foam solid. ¹H NMR (400 MHz, DMSO-d₆) δ 8.31 (s, 1H), 7.88 (s, 1H), 7.63 (d, J=8.0 Hz, 2H), 7.24 (s, 1H), 7.10 (s, 2H), 6.27 (s, 1H), 5.74 (s, 1H), 4.45 (s, 1H), 4.06 (d, J=14.0 Hz, 1H), 3.90 (d, J=13.8 Hz, 1H), 3.70-3.41 (m, 2H), 3.17 (s, 1H), 3.03 (d, J=43.8 Hz, 1H), 2.01 (s, 6H), 1.80 (q, J=13.0, 12.2 Hz, 1H), 1.71-1.56 (m, 1H), 1.42 (d, J=9.8 Hz, 9H). ESI-MS m/z calc. 593.23083, found 594.0 (M+1)⁺; Retention time: 1.52 minutes, LC method A.

### Step 6: tert-Butyl 12-(2,6-dimethylphenyl)-2,8,8-trioxo-15-oxa-8λ⁶-thia-1,9,11,18,23-pentaazatetracyclo[14.5.1.13,7.110,14]tetracosa-3(24),4,6,10,12,14(23)-hexaene-18-carboxylate

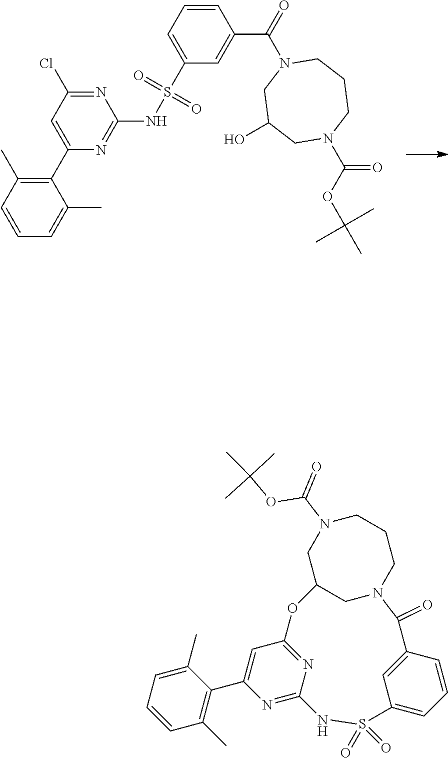 [SMILES: Cc1cccc(C)c1-c1cc(Cl)nc(NS(=O)(=O)c2cccc(C(=O)N3CCCN(C(=O)OC(C)(C)C)CC(O)C3)c2)n1 ; Cc1cccc(C)c1-c1cc2nc(n1)NS(=O)(=O)c1cccc(c1)C(=O)N1CCCN(C(=O)OC(C)(C)C)CC(C1)O2]

To a solution of tert-butyl 5-[3-[[4-chloro-6-(2,6-dimethylphenyl)pyrimidin-2-yl]sulfamoyl]benzoyl]-3-hydroxy-1,5-diazocane-1-carboxylate (1.25 g, 1.88 mmol) in anhydrous dimethylformamide (10 mL) was added 60% suspension sodium hydride in mineral oil (700 mg, 17.5 mmol) in several portions at OC. The reaction mixture was stirred at room temperature for 4 hours and then quenched with 10% aqueous citric acid solution (20 mL). Extracted with ethyl acetate (3×30 mL) and the combined organic layers were washed with brine (3×50 mL), dried over anhydrous sodium sulfate and concentrated. The residue was purified by HPLC to get tert-butyl 12-(2,6-dimethylphenyl)-2,8,8-trioxo-15-oxa-8λ⁶-thia-1,9,11,18,23-pentaazatetracyclo[14.5.1.13,7.110,14]tetracosa-3(24),4,6,10,12,14(23)-hexaene-18-carboxylate (760 mg, 68%) as white solid. ¹H NMR (250 MHz, CDCl₃) δ 8.51 (s, 1H), 7.68 (s, 2H), 7.48 (s, 1H), 7.29-7.14 (m, 1H), 7.05 (s, 2H), 6.40 (s, 1H), 6.08 (d, J=10.6 Hz, 1H), 4.76 (d, J=14.0 Hz, 1H), 4.43-4.05 (m, 2H), 3.78 (dd, J=41.7, 14.1 Hz, 1H), 3.47-2.68 (m, 5H), 2.41-2.08 (m, 1H), 2.02 (s, 6H), 1.74 (dd, J=27.6, 14.7 Hz, 1H), 1.49 (s, 9H). ESI-MS m/z calc. 593.2, found 594.5 (M+1)⁺; Retention time: 2.24 minutes, LC method T.

### Step 7: 12-(2,6-Dimethylphenyl)-15-oxa-8λ⁶-thia-1,9,11,18,23-pentaazatetracyclo[14.5.1.13,7.110,14]tetracosa-3(24),4,6,10,12,14(23)-hexaene-2,8,8-trione

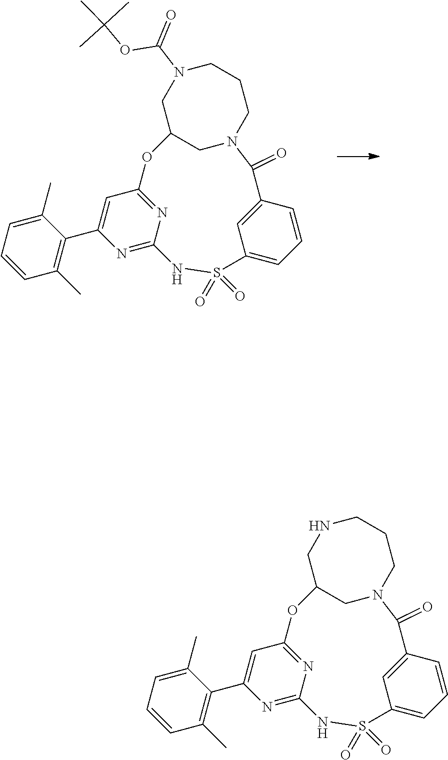 [SMILES: Cc1cccc(C)c1-c1cc2nc(n1)NS(=O)(=O)c1cccc(c1)C(=O)N1CCCN(C(=O)OC(C)(C)C)CC(C1)O2 ; Cc1cccc(C)c1-c1cc2nc(n1)NS(=O)(=O)c1cccc(c1)C(=O)N1CCCNCC(C1)O2]

To a solution of tert-butyl 12-(2,6-dimethylphenyl)-2,8,8-trioxo-15-oxa-8λ⁶-thia-1,9,11,18,23-pentaazatetracyclo[14.5.1.13,7.110,14]tetracosa-3(24),4,6,10,12,14(23)-hexaene-18-carboxylate (8.27 g, 14.1 mmol) in DCM (10 mL) was added 4M HCl in dioxane (30 mL) at 0° C. and the reaction mixture was stirred at room temperature for 1 hour. The solvents were removed under reduce pressure and then ether (15 mL) was added to the white solid. It was filtered to give 12-(2,6-dimethylphenyl)-15-oxa-8λ⁶-thia-1,9,11,18,23-pentaazatetracyclo[14.5.1.13,7.110,14]tetracosa-3(24),4,6,10,12,14(23)-hexaene-2,8,8-trione (hydrochloride salt) (640 mg, 99%) as a white solid. ¹H NMR (250 MHz, DMSO) δ 10.33 (s, 1H), 9.28 (s, 1H), 8.80 (s, 1H), 7.91 (d, J=6.8 Hz, 1H), 7.64 (d, J=6.2 Hz, 2H), 7.36-7.22 (m, 1H), 7.13 (d, J=7.5 Hz, 2H), 6.34 (s, 1H), 5.79 (d, J=10.8 Hz, 1H), 4.37 (d, J=12.9 Hz, 1H), 3.91-3.52 (m, 4H), 3.48-3.01 (m, 4H), 2.05 (m, 8H). ESI-MS m/z calc. 493.2, found 494.5 (M+1)⁺; Retention time: 1.31 minutes, LC method T.

### Step 8: 18-Benzyl-12-(2,6-dimethylphenyl)-15-oxa-8λ⁶-thia-1,9,11,18,23-pentaazatetracyclo[14.5.1.13,7.110,14]tetracosa-3,5,7(24),10,12,14(23)-hexaene-2,8,8-trione (Compound 205)

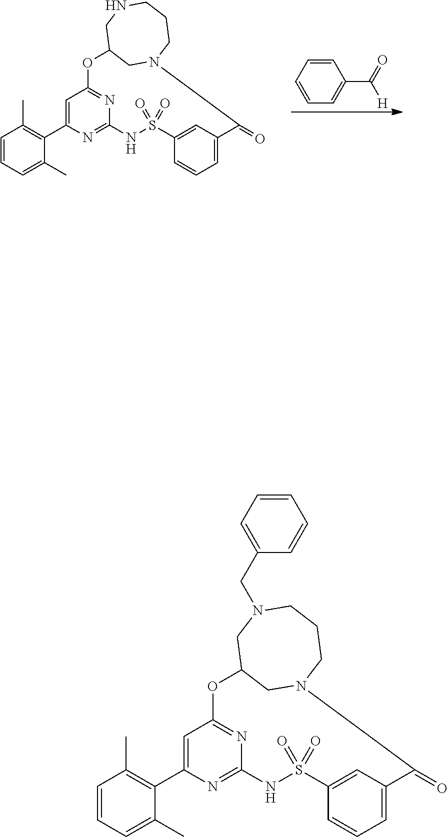 [SMILES: Cc1cccc(C)c1-c1cc2nc(n1)NS(=O)(=O)c1cccc(c1)C(=O)N1CCCNCC(C1)O2 ; O=Cc1ccccc1 ; Cc1cccc(C)c1-c1cc2nc(n1)NS(=O)(=O)c1cccc(c1)C(=O)N1CCCN(Cc3ccccc3)CC(C1)O2]

12-(2,6-dimethylphenyl)-8-imino-15-oxa-8λ⁶-thia-1,9,11,18,22-pentaazatetracyclo[14.4.1.13,7.110,14]tricosa-3,5,7(23),10,12,14(22)-hexaene-2,8-dione (100 mg, 0.2024 mmol) (first diastereomer) was combined with benzaldehyde (42 μL, 0.4132 mmol), acetic acid (60 μL, 1.055 mmol), and sodium triacetoxyborohydride (250 mg, 1.180 mmol) in DCE (2 mL), and stirred for 1 hour at room temperature. was then added and the reaction was stirred for an additional hour at room temperature, then diluted with methanol, filtered, and purified by reverse phase HPLC (10-60% ACN in water, with HCl, 15 min run) to give 18-benzyl-12-(2,6-dimethylphenyl)-15-oxa-8λ⁶-thia-1,9,11,18,23-pentaazatetracyclo[14.5.1.13,7.110,14]tetracosa-3,5,7(24),10,12,14(23)-hexaene-2,8,8-trione (69.3 mg, 59%)¹H NMR (400 MHz, DMSO-d₆) δ 12.95 (s, 1H), 10.56 (s, 1H), 8.50-8.42 (m, 1H), 7.87 (s, 1H), 7.63 (s, 4H), 7.43 (s, 2H), 7.29 (s, 1H), 7.23-7.17 (m, 1H), 7.10-7.04 (m, 2H), 6.30 (s, 1H), 6.15-5.83 (m, 1H), 5.71 (s, 1H), 5.55 (s, 1H), 4.56 (s, 2H), 4.37 (s, 1H), 4.07 (s, 1H), 2.12-1.86 (m, 11H). ESI-MS m/z calc. 583.22534, found 584.0 (M+1)⁺; Retention time: 1.03 minutes (3 min run), LC method A.

### Example 104: Preparation of Compound 206

### Step 1: 18-(3,3-Dimethylbutyl)-12-(2,6-dimethylphenyl)-15-oxa-8λ⁶-thia-1,9,11,18,23-pentaazatetracyclo[14.5.1.13,7.110,14]tetracosa-3,5,7(24),10,12,14(23)-hexaene-2,8,8-trione (Compound 206)

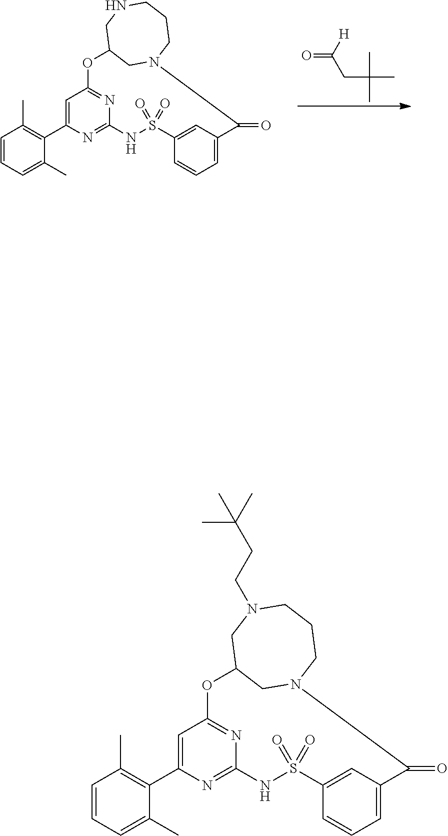

12-(2,6-Dimethylphenyl)-8-imino-15-oxa-8λ⁶-thia-1,9,11,18,22-pentaazatetracyclo[14.4.1.13,7.110,14]tricosa-3,5,7(23),10,12,14(22)-hexaene-2,8-dione (hydrochloride salt) (50 mg, 0.09424 mmol) 3,3-dimethylbutanal (20 μL, 0.1593 mmol), acetic acid (25 μL, 0.4396 mmol), in DCE (1 mL), and stirred for 1 hour at room temperature. Sodium cyanoborohydride (30 mg, 0.4774 mmol) was then added and the reaction was stirred for an additional hour at room temperature. The reaction was repeated and sodium triacetoxyborohydride (100 mg, 0.4718 mmol) was used. The reaction was then diluted with methanol, filtered, and purified by reverse phase HPLC (10-60% ACN in water, with HCl, 15 min run) to give 18-(3,3-dimethylbutyl)-12-(2,6-dimethylphenyl)-15-oxa-8λ⁶-thia-1,9,11,18,23-pentaazatetracyclo[14.5.1.13,7.110,14]tetracosa-3,5,7(24),10,12,14(23)-hexaene-2,8,8-trione (1.7 mg, 3%). ¹H NMR (400 MHz, DMSO-d₆) δ 10.07 (s, 1H), 8.60 (s, 1H), 7.94 (s, 1H), 7.69 (s, 2H), 7.27 (t, J=7.7 Hz, 1H), 7.18-7.11 (m, 2H), 6.37 (s, 1H), 5.79 (s, 1H), 4.45-4.36 (m, 1H), 4.07-3.99 (m, 1H), 3.90-3.74 (m, 3H), 3.28-3.15 (m, 2H), 2.20-2.12 (m, 2H), 2.05 (s, 7H), 1.71-1.52 (m, 3H), 0.96 (s, 8H), 0.90 (s, 2H). ESI-MS m/z calc. 577.2723, found 578.0 (M+1)⁺; Retention time: 1.12 minutes. LC method A.

### Example 105: Preparation of (3R,7R)-19-(2,6-Dimethylphenyl)-8-[2-(oxan-4-yl)ethyl]-5-{spiro[3.4]octan-2-yl}-2-oxa-15λ⁶-thia-5,8,16,18,21-pentaazatetracyclo[15.3.1.110,14.03,7]docosa-1(20),10(22),11,13,17(21),18-hexaene-9,15,15-trione

### Step 1: 3-[[4-[(3R,4R)-1-tert-Butoxycarbonyl-4-(2-tetrahydropyran-4-ylethylamino)pyrrolidin-3-yl]oxy-6-(2,6-dimethylphenyl)pyrimidin-2-yl]sulfamoyl]benzoic acid

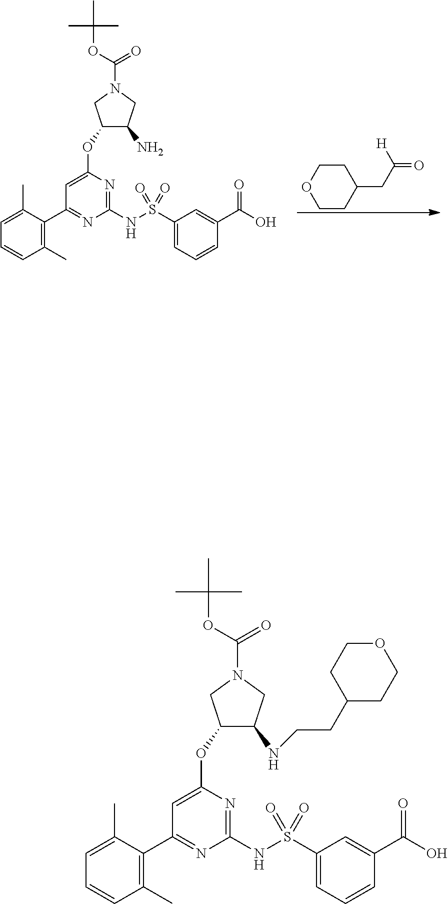 [SMILES: Cc1cccc(C)c1-c1cc(O[C@@H]2CN(C(=O)OC(C)(C)C)C[C@H]2N)nc(NS(=O)(=O)c2cccc(C(=O)O)c2)n1 ; O=CCC1CCOCC1 ; Cc1cccc(C)c1-c1cc(O[C@@H]2CN(C(=O)OC(C)(C)C)C[C@H]2NCCC2CCOCC2)nc(NS(=O)(=O)c2cccc(C(=O)O)c2)n1]

A solution of 3-[[4-[(3R,4R)-4-amino-1-tert-butoxycarbonyl-pyrrolidin-3-yl]oxy-6-(2,6-dimethylphenyl)pyrimidin-2-yl]sulfamoyl]benzoic acid (0.12 g, 0.2056 mmol), 2-tetrahydropyran-4-ylacetaldehyde (52 mg, 0.4057 mmol), and sodium triacetoxyborohydride (0.13 g, 0.6134 mmol) in dichloromethane (1 mL) was stirred for 19 hours. The reaction was stirred with methanol, the volatiles were removed under vacuum, and the residue was purified by reverse-phase HPLC-MS (1%-99% acetonitrile/water (5 mM HCl)) to give a mixture containing product and deprotected product. The mixture was re-purified by reverse-phase HPLC-MS (1%-99% acetonitrile/water) to give 3-[[4-[(3R,4R)-1-tert-butoxycarbonyl-4-(2-tetrahydropyran-4-ylethylamino)pyrrolidin-3-yl]oxy-6-(2,6-dimethylphenyl)pyrimidin-2-yl]sulfamoyl]benzoic acid (16 mg, 11%) as a colorless solid. ESI-MS m/z calc. 695.2989, found 696.4 (M+1)⁺; Retention time: 0.52 minutes, LC method D.

### Step 2: (3R,7R)-19-(2,6-dimethylphenyl)-8-[2-(oxan-4-yl)ethyl]-2-oxa-15λ⁶-thia-5,8,16,18,21-pentaazatetracyclo[15.3.1.110,14.03,7]docosa-1(20),10(22),11,13,17(21),18-hexaene-9,15,15-trione

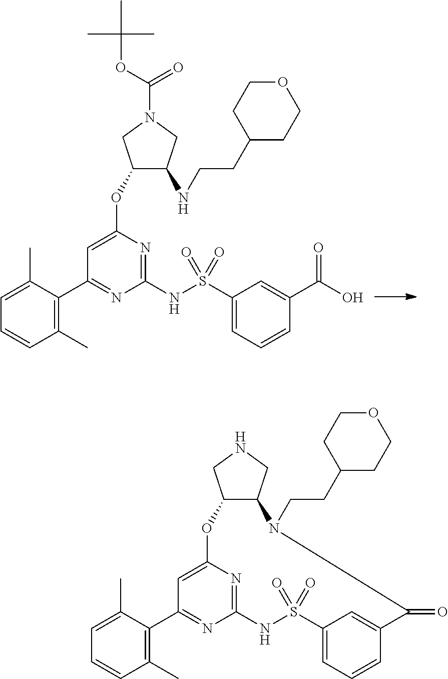 [SMILES: Cc1cccc(C)c1-c1cc(O[C@@H]2CN(C(=O)OC(C)(C)C)C[C@H]2NCCC2CCOCC2)nc(NS(=O)(=O)c2cccc(C(=O)O)c2)n1 ; Cc1cccc(C)c1-c1cc2nc(n1)NS(=O)(=O)c1cccc(c1)C(=O)N(CCC1CCOCC1)[C@@H]1CNC[C@H]1O2]

A solution of 3-[[4-[(3R,4R)-1-tert-butoxycarbonyl-4-(2-tetrahydropyran-4-ylethylamino)pyrrolidin-3-yl]oxy-6-(2,6-dimethylphenyl)pyrimidin-2-yl]sulfamoyl]benzoic acid (16 mg, 0.02299 mmol), HATU (14 mg, 0.03682 mmol), and DIEA (13 μL, 0.07463 mmol) in DMF (2 mL) was stirred for 17 hours. The reaction was diluted with water, acidified with 1 M citric acid, and extracted with ethyl acetate. The combined extracts were washed with brine, dried over sodium sulfate, and evaporated under vacuum. The residue was stirred with HCl (3 mL of 4 M, 12.00 mmol) (in dioxane) for one hour. The solvent was evaporated under vacuum, and the residue was purified by reverse-phase HPLC-MS (1%-99% acetonitrile/water (5 mM HCl)) to give (3R,7R)-19-(2,6-dimethylphenyl)-8-[2-(oxan-4-yl)ethyl]-2-oxa-15λ⁶-thia-5,8,16,18,21-pentaazatetracyclo[15.3.1.110,14.03,7]docosa-1(20),10(22),11,13,17(21),18-hexaene-9,15,15-trione (hydrochloride salt) (7 mg, 50%) as a colorless solid. ESI-MS m/z calc. 577.2359, found 578.3 (M+1)⁺; Retention time: 0.34 minutes, LC method D.

### Step 3: (3R,7R)-19-(2,6-Dimethylphenyl)-8-[2-(oxan-4-yl)ethyl]-5-{spiro[3.4]octan-2-yl}-2-oxa-15λ⁶-thia-5,8,16,18,21-pentaazatetracyclo[15.3.1.110,14.03,7]docosa-1(20),10(22),11,13,17(21),18-hexaene-9,15,15-trione

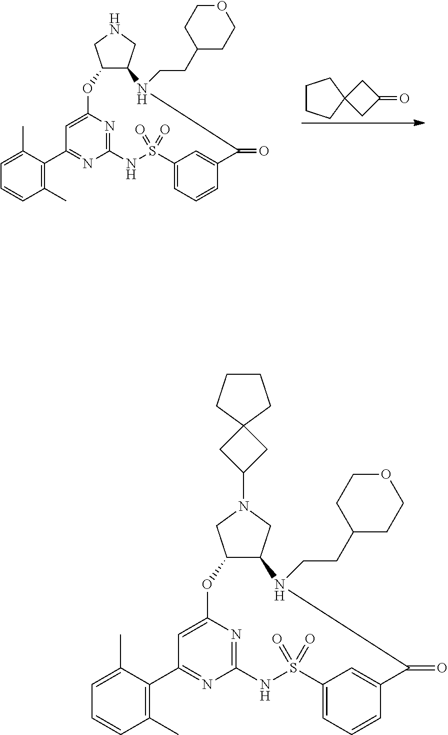 [SMILES: Cc1cccc(C)c1-c1cc2nc(n1)NS(=O)(=O)c1cccc(c1)C(=O)N(CCC1CCOCC1)[C@@H]1CNC[C@H]1O2 ; O=C1CC2(CCCC2)C1 ; Cc1cccc(C)c1-c1cc2nc(n1)NS(=O)(=O)c1cccc(c1)C(=O)N(CCC1CCOCC1)[C@@H]1CN(C3CC4(CCCC4)C3)C[C@H]1O2]

A solution of (3R,7R)-19-(2,6-dimethylphenyl)-8-[2-(oxan-4-yl)ethyl]-2-oxa-15λ⁶-thia-5,8,16,18,21-pentaazatetracyclo[15.3.1.110,14.03,7]docosa-1(20),10(22),11,13,17(21),18-hexaene-9,15,15-trione (hydrochloride salt) (7 mg, 0.01140 mmol), spiro[3.4]octan-2-one (5 μL, 0.04026 mmol), and sodium triacetoxyborohydride (10 mg, 0.04718 mmol) in dichloromethane (0.2 mL) was stirred for two hours. The reaction was stirred with methanol, the volatiles were removed under vacuum, and the residue was purified by reverse-phase HPLC-MS (20%-80% acetonitrile/water (5 mM HCl)) to give (3R,7R)-19-(2,6-dimethylphenyl)-8-[2-(oxan-4-yl)ethyl]-5-{spiro[3.4]octan-2-yl}-2-oxa-15λ⁶-thia-5,8,16,18,21-pentaazatetracyclo[15.3.1.110,14.03,7]docosa-1(20),10(22),11,13,17(21),18-hexaene-9,15,15-trione (hydrochloride salt) (7 mg, 84%) as a colorless solid. ESI-MS m/z calc. 685.3298, found 686.5 (M+1)⁺; Retention time: 1.33 minutes, LC method A.

### Example 106: Preparation of (3R,7R)-19-(2,6-Dimethylphenyl)-8-ethyl-5-{spiro[3.4]octan-2-yl}-2-oxa-15λ⁶-thia-5,8,16,18,21-pentaazatetracyclo[15.3.1.110,14.03,7]docosa-1(20),10(22),11,13,17(21),18-hexaene-9,15,15-trione

### Step 1: 3-[[4-[(3R,4R)-1-tert-Butoxycarbonyl-4-(ethylamino)pyrrolidin-3-yl]oxy-6-(2,6-dimethylphenyl)pyrimidin-2-yl]sulfamoyl]benzoic acid

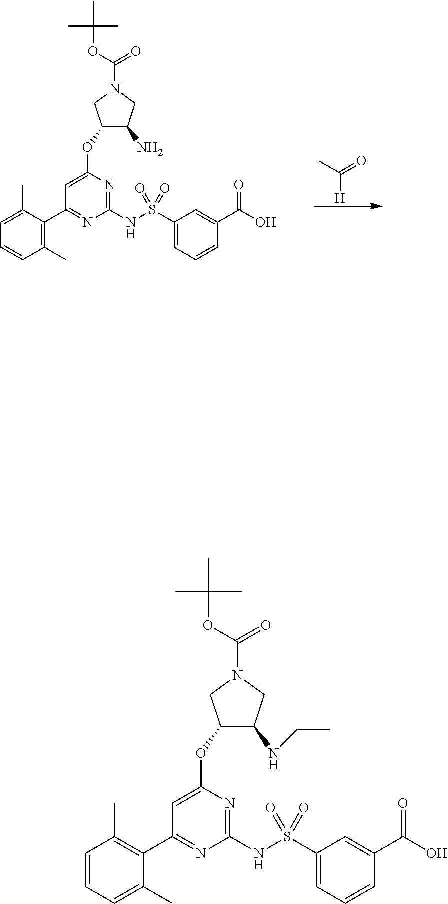

A solution of 3-[[4-[(3R,4R)-4-amino-1-tert-butoxycarbonyl-pyrrolidin-3-yl]oxy-6-(2,6-dimethylphenyl)pyrimidin-2-yl]sulfamoyl]benzoic acid (291.8 mg, 0.5 mmol), acetaldehyde (approximately 24.23 mg, 30.87 μL, 0.5500 mmol), and sodium triacetoxyborohydride (approximately 211.9 mg, 1.000 mmol) in dichloromethane (2.500 mL) was stirred for 18 hours. More acetaldehyde (approximately 24.23 mg, 30.87 μL, 0.5500 mmol) and sodium triacetoxyborohydride (approximately 211.9 mg, 1.000 mmol) were again added, and the reactions were stirred for 22 hours. The solvent was evaporated, and the residue was diluted with water, acidified with 1 M citric acid, and extracted with ethyl acetate. The residue was purified by silica gel column chromatography with 0-8% methanol in dichloromethane to give 3-[[4-[(3R,4R)-1-tert-butoxycarbonyl-4-(ethylamino)pyrrolidin-3-yl]oxy-6-(2,6-dimethylphenyl)pyrimidin-2-yl]sulfamoyl]benzoic acid (44 mg, 14%). ESI-MS m/z calc. 611.2414, found 612.2 (M+1)⁺; Retention time: 0.5 minutes; LC method D.

### Step 2: (3R,7R)-19-(2,6-Dimethylphenyl)-8-ethyl-2-oxa-15λ⁶-thia-5,8,16,18,21-pentaazatetracyclo[15.3.1.110,14.03,7]docosa-1(20),10(22),11,13,17(21),18-hexaene-9,15,15-trione

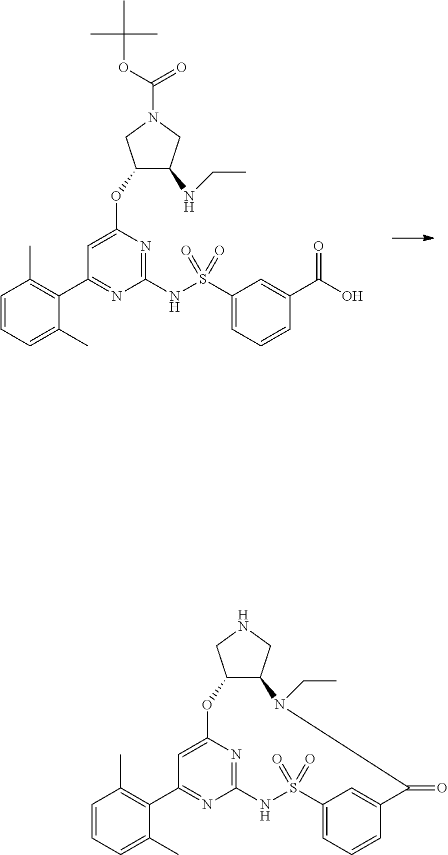 [SMILES: CCN[C@@H]1CN(C(=O)OC(C)(C)C)C[C@H]1Oc1cc(-c2c(C)cccc2C)nc(NS(=O)(=O)c2cccc(C(=O)O)c2)n1 ; CCN1C(=O)c2cccc(c2)S(=O)(=O)Nc2nc(cc(-c3c(C)cccc3C)n2)O[C@@H]2CNC[C@H]21]

A solution of 3-[[4-[(3R,4R)-1-tert-butoxycarbonyl-4-(ethylamino)pyrrolidin-3-yl]oxy-6-(2,6-dimethylphenyl)pyrimidin-2-yl]sulfamoyl]benzoic acid (44 mg, 0.07193 mmol), [[(E)-(1-cyano-2-ethoxy-2-oxo-ethylidene)amino]oxy-tetrahydropyran-4-yl-methylene]-dimethyl-ammonium (Phosphorus Hexafluoride Ion) (49 mg, 0.1147 mmol), and DIEA (38 μL, 0.2182 mmol) in DMF (5 mL) was stirred for three days. The reaction was acidified with 1 M citric acid, diluted with water, and extracted with ethyl acetate. The combined extracts were washed with brine, dried over sodium sulfate, and evaporated under vacuum. The residue was purified by reverse-phase HPLC-MS (1%-99% acetonitrile/water (5 mM HCl)) to give intermediate containing some product. HCl (4 mL of 4 M, 16.00 mmol) (in dioxane) was added, and the reaction was stirred for three hours. The solvent was removed under vacuum to give (3R,7R)-19-(2,6-dimethylphenyl)-8-ethyl-2-oxa-15λ⁶-thia-5,8,16,18,21-pentaazatetracyclo[15.3.1.110,14.03,7]docosa-1(20),10(22),11,13,17(21),18-hexaene-9,15,15-trione (hydrochloride salt) (13 mg, 34%) as a tan solid. ESI-MS m/z calc. 493.17838, found 494.2 (M+1)⁺; Retention time: 0.3 minutes, LC method D.

### Step 3: (3R,7R)-19-(2,6-Dimethylphenyl)-8-ethyl-5-{spiro[3.4]octan-2-yl}-2-oxa-15)⁶-thia-5,8,16,18,21-pentaazatetracyclo[15.3.1.110,14.03,7]docosa-1(20),10(22),11,13,17(21),18-hexaene-9,15,15-trione

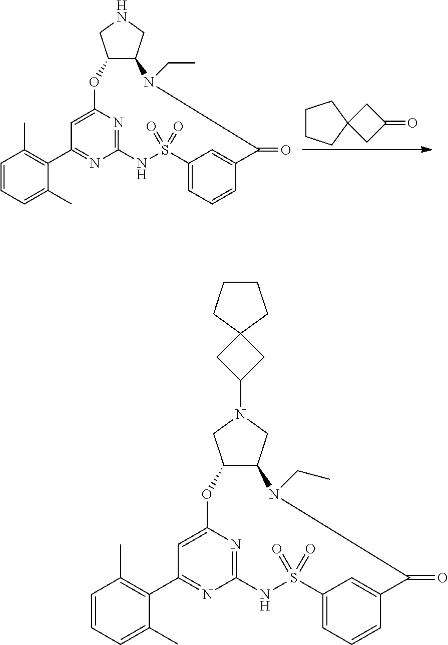

A solution of (3R,7R)-19-(2,6-dimethylphenyl)-8-ethyl-2-oxa-15λ⁶-thia-5,8,16,18,21-pentaazatetracyclo[15.3.1.110,14.03,7]docosa-1(20),10(22),11,13,17(21),18-hexaene-9,15,15-trione (hydrochloride salt) (12 mg, 0.02264 mmol), spiro[3.4]octan-2-one (approximately 8.434 mg, 0.06792 mmol), and sodium triacetoxyborohydride (approximately 19.19 mg, 0.09056 mmol) in dichloromethane (0.3 mL) was stirred for 17 hours. The reaction was stirred with methanol, and the solvent was evaporated. The residue was purified by reverse-phase HPLC-MS (1%-99% acetonitrile/water (5 mM HCl)) to give (3R,7R)-19-(2,6-dimethylphenyl)-8-ethyl-5-{spiro[3.4]octan-2-yl}-2-oxa-15λ⁶-thia-5,8,16,18,21-pentaazatetracyclo[15.3.1.110,14.03,7]docosa-1(20),10(22),11,13,17(21),18-hexaene-9,15,15-trione (hydrochloride salt) (8.4 mg, 61%). ESI-MS m/z calc. 601.2723, found 602.4 (M+1)⁺; Retention time: 1.23 minutes; LC method A.

### Example 108: Preparation of Compound 206

### Step 1: 18-(3,3-Dimethylbutyl)-12-(2,6-dimethylphenyl)-15-oxa-8λ⁶-thia-1,9,11,18,23-pentaazatetracyclo[14.5.1.13,7.110,14]tetracosa-3,5,7(24),10,12,14(23)-hexaene-2,8,8-trione (Compound 206)

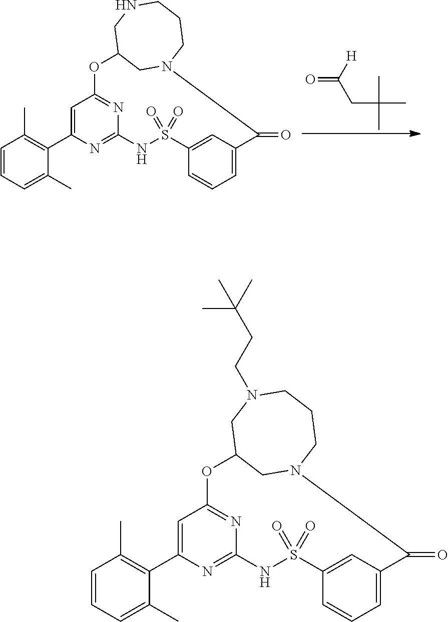

12-(2,6-Dimethylphenyl)-8-imino-15-oxa-8λ⁶-thia-1,9,11,18,22-pentaazatetracyclo[14.4.1.13,7.110,14]tricosa-3,5,7(23),10,12,14(22)-hexaene-2,8-dione (hydrochloride salt) (50 mg, 0.09424 mmol) 3,3-dimethylbutanal (20 μL, 0.1593 mmol), acetic acid (25 μL, 0.4396 mmol), in DCE (1 mL), and stirred for 1 hour at room temperature. Sodium cyanoborohydride (30 mg, 0.4774 mmol) was then added and the reaction was stirred for an additional hour at room temperature. The reaction was repeated and sodium triacetoxyborohydride (100 mg, 0.4718 mmol) was used. The reaction was then diluted with methanol, filtered, and purified by reverse phase HPLC (10-60% ACN in water, with HCl, 15 min run) to give 18-(3,3-dimethylbutyl)-12-(2,6-dimethylphenyl)-15-oxa-8λ⁶-thia-1,9,11,18,23-pentaazatetracyclo[14.5.1.13,7.110,14]tetracosa-3,5,7(24),10,12,14(23)-hexaene-2,8,8-trione (1.7 mg, 3%)¹H NMR (400 MHz, DMSO-d₆) δ 10.08 (s, 1H), 8.61 (s, 1H), 7.94 (s, 1H), 7.67 (s, 2H), 7.26 (s, 1H), 7.13 (s, 2H), 6.37 (s, 1H), 5.81 (s, 1H), 4.42 (s, 1H), 4.05 (s, 1H), 3.83 (s, 2H), 3.22 (s, 1H), 2.15 (d, J=6.1 Hz, 2H), 2.05 (s, 7H), 1.68-1.56 (m, 2H), 0.96 (s, 9H), 0.90 (s, 2H). ESI-MS m/z calc. 577.2723, found 578.0 (M+1)⁺; Retention time: 1.12 minutes. LC method A.

### Example 109: Preparation of Compound 207

### Step 1: tert-Butyl 4-[2-chloro-6-(2,6-dimethylphenyl)pyrimidin-4-yl]oxy-3,4-dihydro-1H-isoquinoline-2-carboxylate

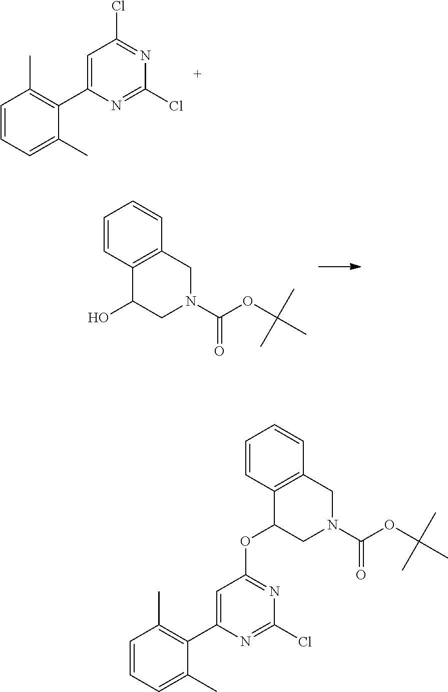

A solution of tert-butyl 4-hydroxy-3,4-dihydro-1H-isoquinoline-2-carboxylate (1.25 g, 5.014 mmol) in DMF (25 mL) was cooled in an ice bath, and sodium hydride (0.20 g of 60% w/w, 5.000 mmol) was added. After 15 minutes 2,4-dichloro-6-(2,6-dimethylphenyl)pyrimidine (1.26 g, 4.978 mmol) was added, and the reaction was allowed to slowly warm to room temperature and stir for three days. The reaction was diluted with water and extracted with ethyl acetate. The combined extracts were washed with brine and water, dried over sodium sulfate, and evaporated. The residue was purified by silica gel column chromatography with 0-20% ethyl acetate in hexanes to give tert-butyl 4-[2-chloro-6-(2,6-dimethylphenyl)pyrimidin-4-yl]oxy-3,4-dihydro-1H-isoquinoline-2-carboxylate (1.56 g, 67%) as a colorless solid. ESI-MS m/z calc. 465.18192, found 466.3 (M+1)⁺; Retention time: 0.86 minutes, LC method D.

### Step 2: 4-[2-Chloro-6-(2,6-dimethylphenyl)pyrimidin-4-yl]oxy-1,2,3,4-tetrahydroisoquinoline

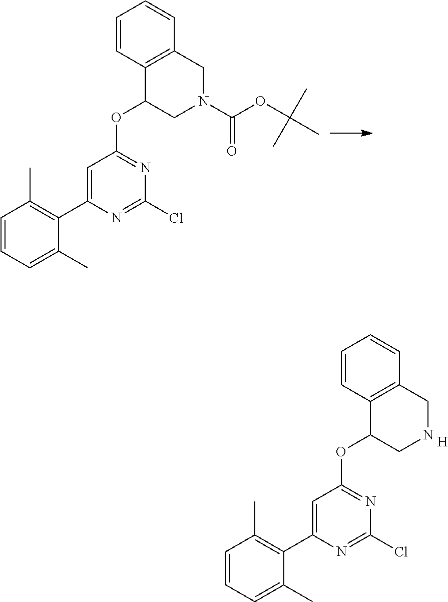

A solution of tert-butyl 4-[2-chloro-6-(2,6-dimethylphenyl)pyrimidin-4-yl]oxy-3,4-dihydro-1H-isoquinoline-2-carboxylate (2.26 g, 4.850 mmol) in HCl (16 mL of 4 M, 64.00 mmol) (in dioxane) was stirred for 15 hours. The solvent was evaporated and the resulting solid was triturated with diethyl ether and dried under vacuum to give 4-[2-chloro-6-(2,6-dimethylphenyl)pyrimidin-4-yl]oxy-1,2,3,4-tetrahydroisoquinoline (hydrochloride salt) (1.76 g, 90%) as a light-yellow solid. ESI-MS m/z calc. 365.1295, found 366.2 (M+1)⁺; Retention time: 0.49 minutes, LC method D.

### Step 3: 5-[4-[2-Chloro-6-(2,6-dimethylphenyl)pyrimidin-4-yl]oxy-3,4-dihydro-1H-isoquinoline-2-carbonyl]-1-methyl-pyrrole-3-sulfonamide

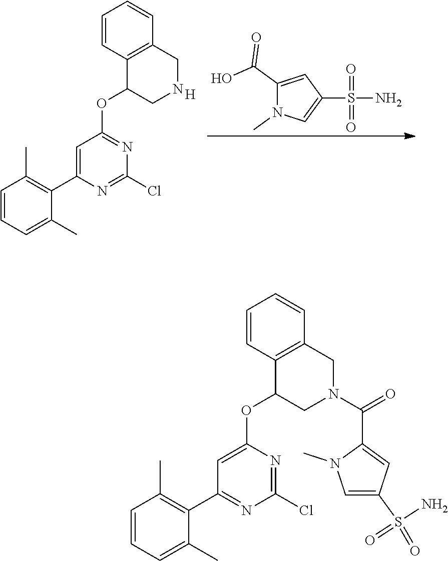

A solution of 1-methyl-4-sulfamoyl-pyrrole-2-carboxylic acid (50 mg, 0.2449 mmol), N,N′-diisopropylcarbodiimide (38 μL, 0.2427 mmol), and sodium bicarbonate (approximately 84.01 mg, 1.000 mmol) in dichloromethane (1.207 mL) was stirred for two hours, and 4-[2-chloro-6-(2,6-dimethylphenyl)pyrimidin-4-yl]oxy-1,2,3,4-tetrahydroisoquinoline (hydrochloride salt) (80.46 mg, 0.2 mmol) was added. The reaction was stirred for 18 hours, diluted with water, and extracted with ethyl acetate. The combined extracts were washed with water, dried over sodium sulfate, and evaporated. The residue was purified by silica gel column chromatography with 0-4% methanol in dichloromethane to give 5-[4-[2-chloro-6-(2,6-dimethylphenyl)pyrimidin-4-yl]oxy-3,4-dihydro-1H-isoquinoline-2-carbonyl]-1-methyl-pyrrole-3-sulfonamide (87 mg, 79%). ESI-MS m/z calc. 551.1394, found 552.3 (M+1)⁺; Retention time: 0.73 minutes; LC method D.

### Step 4: 11-(2,6-Dimethylphenyl)-4-methyl-14-oxa-7λ⁶-thia-1,4,8,10,24-pentaazapentacyclo[13.7.1.13,6.19,13.016,21]pentacosa-3(25),5,9(24),10,12,16(21),17,19-octaene-2,7,7-trione (Compound 207)

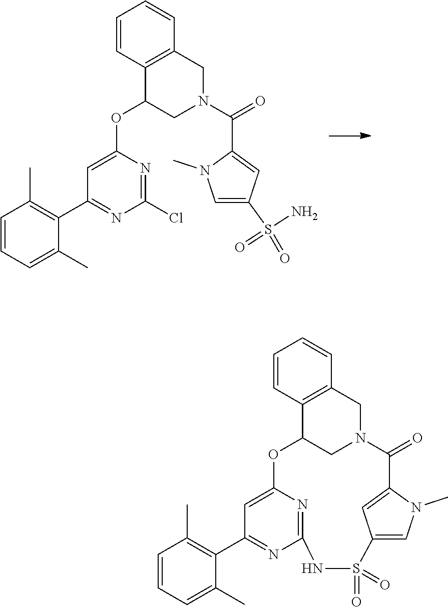 [SMILES: Cc1cccc(C)c1-c1cc(OC2CN(C(=O)c3cc(S(N)(=O)=O)cn3C)Cc3ccccc32)nc(Cl)n1 ; Cc1cccc(C)c1-c1cc2nc(n1)NS(=O)(=O)c1cc(n(C)c1)C(=O)N1Cc3ccccc3C(C1)O2]

A solution of 5-[4-[2-chloro-6-(2,6-dimethylphenyl)pyrimidin-4-yl]oxy-3,4-dihydro-1H-isoquinoline-2-carbonyl]-1-methyl-pyrrole-3-sulfonamide (87 mg, 0.1576 mmol), Palladium (II) acetate (8 mg, 0.03563 mmol), 4,5-bis(diphenylphosphino)-9,9-dimethylxanthene (19 mg, 0.03284 mmol), and cesium carbonate (0.11 g, 0.3376 mmol) in dioxane (8 mL) was degassed with a stream of nitrogen and stirred at 100° C. for 16 hours. The reaction was filtered and purified using a reverse phase HPLC-MS method using a Luna C₁₈ (2) column (75×30 mm, 5 μm particle size) sold by Phenomenex (pn: 00C-4252-U0-AX), and a dual gradient run from 15-75% mobile phase B over 15.0 minutes. Mobile phase A=H₂O (5 mM HCl). Mobile phase B=CH₃CN. Flow rate=50 mL/min, and column temperature=25° C.; to give 11-(2,6-dimethylphenyl)-4-methyl-14-oxa-7λ⁶-thia-1,4,8,10,24-pentaazapentacyclo[13.7.1.13,6.19,13.016,21]pentacosa-3(25),5,9(24),10,12,16(21),17,19-octaene-2,7,7-trione (25 mg, 30%), obtained as a colorless solid. ¹H NMR (400 MHz, DMSO-d₆) δ 7.65-7.55 (m, 2H), 7.42-7.28 (m, 4H), 7.28-7.19 (m, 1H), 7.12 (d, J=7.6 Hz, 2H), 6.47 (s, 1H), 6.19 (dd, J=10.6, 5.0 Hz, 1H), 5.16 (d, J=17.1 Hz, 1H), 4.39 (d, J=17.1 Hz, 1H), 3.76-3.64 (m, 4H), 3.05 (dd, J=12.8, 10.6 Hz, 1H), 2.07 (s, 7H). ESI-MS m/z calc. 515.1627, found 516.3 (M+1)⁺; Retention time: 1.61 minutes; (LC method A).

### Example 110: Preparation of Compound 208

### Step 1: 3-[[4-[2-Chloro-6-(2,6-dimethylphenyl)pyrimidin-4-yl]oxy-3,4-dihydro-1H-isoquinolin-2-yl]methyl]benzenesulfonamide

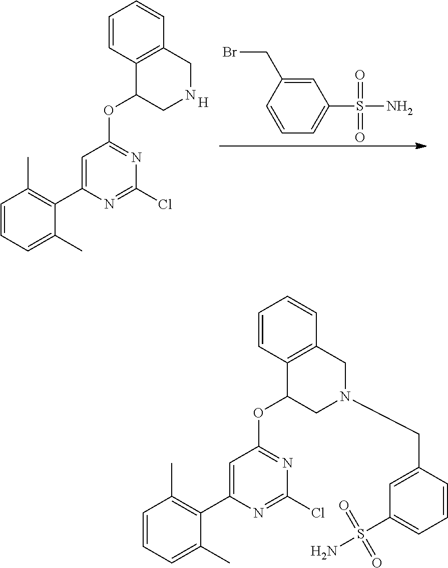

A mixture of 4-[2-chloro-6-(2,6-dimethylphenyl)pyrimidin-4-yl]oxy-1,2,3,4-tetrahydroisoquinoline (hydrochloride salt) (0.27 g, 0.6711 mmol), 3-(bromomethyl)benzenesulfonamide (0.17 g, 0.6797 mmol), and sodium bicarbonate (0.28 g, 3.333 mmol) in DMF (3 mL) was stirred for 19 hours. The reaction was diluted with water and extracted with ethyl acetate. The combined extracts were washed with brine and water, dried over sodium sulfate, and evaporated. The residue was purified by silica gel column chromatography with 0-4% methanol in dichloromethane to give 3-[[4-[2-chloro-6-(2,6-dimethylphenyl)pyrimidin-4-yl]oxy-3,4-dihydro-1H-isoquinolin-2-yl]methyl]benzenesulfonamide (0.29 g, 81%) as a colorless solid. ESI-MS m/z calc. 534.14923, found 535.3 (M+1)⁺; Retention time: 0.59 minutes, LC method D.

### Step 2: 12-(2,6-Dimethylphenyl)-15-oxa-8λ⁶-thia-1,9,11,25-tetraazapentacyclo[14.7.1.13,7.110,14.017,22]hexacosa-3(26),4,6,10,12,14(25),17(22),18,20-nonaene-8,8-dione (Compound 208)

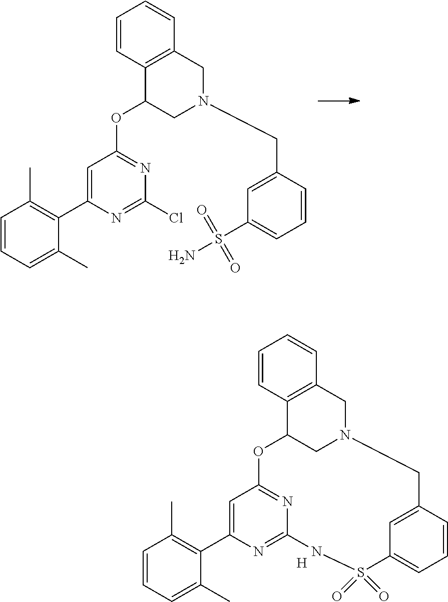

A solution of 3-[[4-[2-chloro-6-(2,6-dimethylphenyl)pyrimidin-4-yl]oxy-3,4-dihydro-1H-isoquinolin-2-yl]methyl]benzenesulfonamide (68 mg, 0.1271 mmol), Palladium (II) acetate (6 mg, 0.02672 mmol), 4,5-bis(diphenylphosphino)-9,9-dimethylxanthene (15 mg, 0.02592 mmol), and cesium carbonate (0.12 g, 0.3683 mmol) in dioxane (7 mL) was degassed with a stream of nitrogen and stirred in a sealed vial at 100° C. for 15 hours. The reaction was cooled to room temperature, filtered, and concentrated. The residue was purified using a reverse phase HPLC-MS method using a Luna C₁₈ (2) column (75×30 mm, 5 μm particle size) sold by Phenomenex (pn: 00C-4252-U0-AX), and a dual gradient run from 15-75% mobile phase B over 15.0 minutes. Mobile phase A=H₂O (5 mM HCl). Mobile phase B=CH₃CN. Flow rate=50 mL/min, and column temperature=25° C. to give 12-(2,6-dimethylphenyl)-15-oxa-8λ⁶-thia-1,9,11,25-tetraazapentacyclo[14.7.1.13,7.110,14.017,22]hexacosa-3(26),4,6,10,12,14(25),17(22),18,20-nonaene-8,8-dione (hydrochloride salt) (32 mg, 47%) obtained as a colorless solid. ESI-MS m/z calc. 498.17255, found 499.3 (M+1)⁺; Retention time: 1.12 minutes, LC method A.

### Example 111: Preparation of Compound 209

### Step 1: tert-Butyl (3R)-3-[methoxy(methyl)carbamoyl]morpholine-4-carboxylate

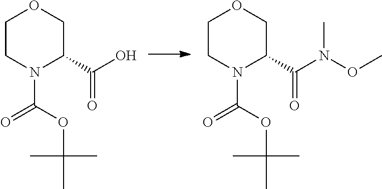 [SMILES: CC(C)(C)OC(=O)N1CCOC[C@@H]1C(=O)O ; CON(C)C(=O)[C@H]1COCCN1C(=O)OC(C)(C)C]

To a solution of (3R)-4-tert-butoxycarbonylmorpholine-3-carboxylic acid (3.01 g, 13.02 mmol), N-methoxymethanamine (hydrochloride salt) (1.59 g, 16.30 mmol) and 1-hydroxybenzotriazole (2.2 g, 16.28 mmol) in DMF (46 mL) was added 3-(ethyliminomethyleneamino)-N,N-dimethyl-propan-1-amine (hydrochloride salt) (3.3 g, 17.21 mmol) followed by 4-methylmorpholine (5.75 mL, 52.30 mmol) and the reaction mixture was stirred at room temperature for 16 hours. The reaction mixture was poured into water, the pH was adjusted to 4-5 with 1N HCl and extracted with EtOAc (2×). The combined organic phase was washed with 0.1N HCl (2×), saturated aqueous sodium carbonate, water, brine, dried over sodium sulfate and evaporated to dryness. Purification by column chromatography (120 g Silica; 0 to 50% EtOAc in hexanes) gave tert-butyl (3R)-3-[methoxy(methyl)carbamoyl]morpholine-4-carboxylate (2.5 g, 70%) as a white solid. ESI-MS m/z calc. 274.15286, found 275.0 (M+1)⁺; Retention time: 0.45 minutes, LC method D.

### Step 2: tert-Butyl (3R)-3-(1-hydroxy-2-phenyl-ethyl)morpholine-4-carboxylate

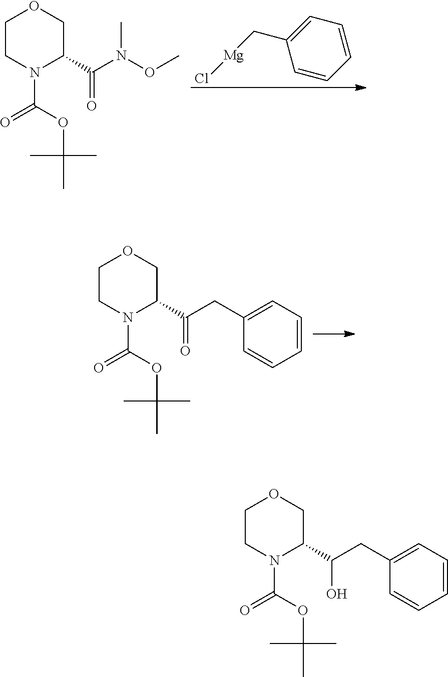

To a solution of tert-butyl (3R)-3-[methoxy(methyl)carbamoyl]morpholine-4-carboxylate (270 mg, 0.9843 mmol) in THE (3 mL) at 0° C. was added benzyl(chloro)magnesium (2 M in THF) (550 μL of 2 M, 1.100 mmol) dropwise, the cooling bath was removed and the reaction mixture was stirred at room temperature for 2 hours. The reaction mixture was quenched with ice, poured into water, pH adjusted to ˜5 with 1M HCl and extracted with EtOAc (3×). Organics were combined, washed with brine, dried over sodium sulfate, and evaporated to dryness. The residue was taken up in MeOH (5 mL) and the solution cooled to 0° C. Sodium borohydride (42 mg, 1.110 mmol) was then added and the reaction mixture stirred at room temperature for 90 min. The reaction mixture was poured into water and extracted with EtOAc (3×). Organics were combined, washed with brine, dried over sodium sulfate and evaporated to dryness. Purification by column chromatography (24 g silica; 0-40% EtOAc in hexanes) gave tert-butyl (3R)-3-(1-hydroxy-2-phenyl-ethyl)morpholine-4-carboxylate (200 mg, 66%) as a foam. ESI-MS m/z calc. 307.17834, found 308.1 (M+1)⁺; Retention time: 0.58 minutes, LC method D.

### Step 3: 1-[(3R)-4-Benzylmorpholin-3-yl]-2-phenyl-ethanol

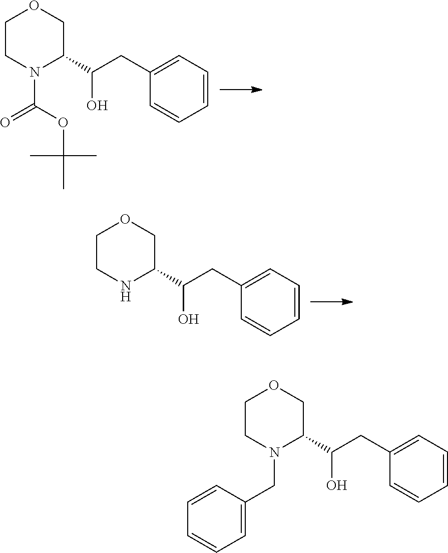 [SMILES: CC(C)(C)OC(=O)N1CCOC[C@@H]1C(O)Cc1ccccc1 ; OC(Cc1ccccc1)[C@H]1COCCN1 ; OC(Cc1ccccc1)[C@H]1COCCN1Cc1ccccc1]

To a solution of tert-butyl (3R)-3-(1-hydroxy-2-phenyl-ethyl)morpholine-4-carboxylate (200 mg, 0.6507 mmol) in DCM (4 mL) was added TFA (250 μL, 3.245 mmol) and the reaction mixture stirred at room temperature for 3 hours. More TFA (250 μL, 3.245 mmol) was added and stirred at room temperature for 1 hour. The reaction mixture was the evaporated and co-evaporated with EtOH (3×). The residue was taken up in EtOH (2 mL) and water (2 mL). To this solution was added potassium carbonate (205 mg, 1.483 mmol) followed by benzyl bromide (85 μL, 0.7147 mmol) and the reaction mixture was stirred at room temperature for 16 hours. The reaction mixture was poured into water and extracted with EtOAc (3×). Organics were combined, washed with brine and evaporated to dryness. Purification by flash chromatography (12 g 0-40% EtOAC in hexanes) gave 1-[(3R)-4-benzylmorpholin-3-yl]-2-phenyl-ethanol (75 mg, 39%) as a clear oil. ESI-MS m/z calc. 297.17288, found 298.2 (M+1)⁺; Retention time: 0.37 minutes, LC method D.

### Step 4: 3-[[4-[1-[(3R)-4-Benzylmorpholin-3-yl]-2-phenyl-ethoxy]-6-(2,6-dimethylphenyl)pyrimidin-2-yl]sulfamoyl]benzoic acid

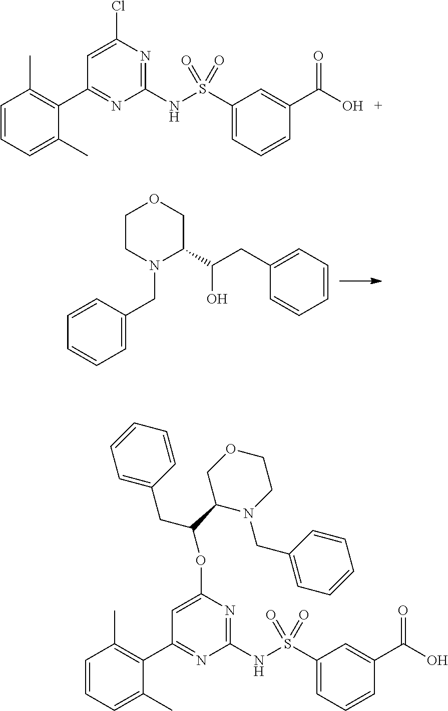

To a solution of 1-[(3R)-4-benzylmorpholin-3-yl]-2-phenyl-ethanol (75 mg, 0.2522 mmol) and 3-[[4-chloro-6-(2,6-dimethylphenyl)pyrimidin-2-yl]sulfamoyl]benzoic acid (153 mg, 0.3661 mmol) in THE (2 mL) at 0° C. was added sodium tert-butoxide (101 mg, 1.051 mmol) and the reaction mixture was slowly allowed to warm to room temperature over 16 hours. At this point, more sodium tert-butoxide (50 mg, 0.5203 mmol) was added and the reaction mixture stirred for 2 hours. The reaction was diluted with 1:1 DMSO:MeOH and a few drops of water, filtered and purification by HPLC (1-99% ACN in water (HCl modifier)) gave 3-[[4-[1-[(3R)-4-benzylmorpholin-3-yl]-2-phenyl-ethoxy]-6-(2,6-dimethylphenyl)pyrimidin-2-yl]sulfamoyl]benzoic acid (hydrochloride salt) (137 mg, 17%). ESI-MS m/z calc. 678.2512, found 679.5 (M+1)⁺; Retention time: 0.55 minutes, LC method D.

### Step 5: 3-[[4-(2,6-Dimethylphenyl)-6-[1-[(3R)-morpholin-3-yl]-2-phenyl-ethoxy]pyrimidin-2-yl]sulfamoyl]benzoic acid

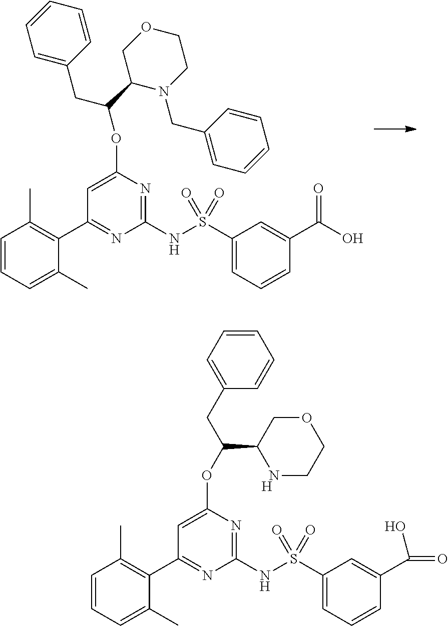

To 3-[[4-[1-[(3R)-4-benzylmorpholin-3-yl]-2-phenyl-ethoxy]-6-(2,6-dimethylphenyl)pyrimidin-2-yl]sulfamoyl]benzoic acid (hydrochloride salt) (180 mg, 0.1183 mmol) and palladium (42 mg of 10% w/w, 0.03947 mmol) was added MeOH (1 mL) and aqueous HCl (58 μL of 37% w/v, 0.5886 mmol) and the reaction mixture stirred under a balloon of hydrogen for 1 hours. The reaction mixture was filtered, solvents evaporated to dryness to give 3-[[4-(2,6-dimethylphenyl)-6-[1-[(3R)-morpholin-3-yl]-2-phenyl-ethoxy]pyrimidin-2-yl]sulfamoyl]benzoic acid (hydrochloride salt) (110 mg, 77%) as an off white solid. ESI-MS m/z calc. 588.2043, found 589.4 (M+1)⁺; Retention time: 0.48 minutes, LC method D.

### Step 6: (8R)-9-Benzyl-13-(2,6-dimethylphenyl)-6,10-dioxa-17λ⁶-thia-3,14,16,23-tetraazatetracyclo[16.3.1.111,15.03,8]tricosa-1(22),11,13,15(23),18,20-hexaene-2,17,17-trione (Compound 209)

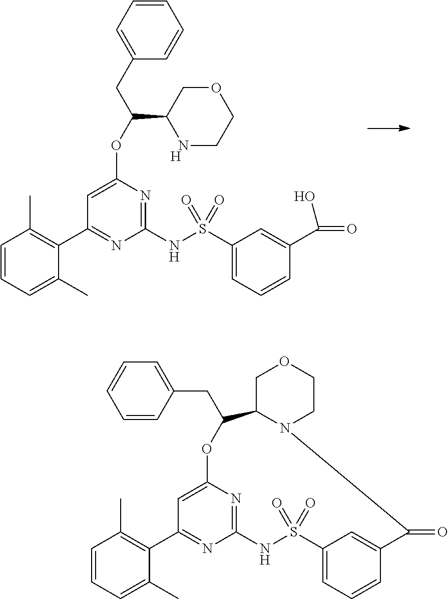

To a solution of 3-[[4-(2,6-dimethylphenyl)-6-[1-[(3R)-morpholin-3-yl]-2-phenyl-ethoxy]pyrimidin-2-yl]sulfamoyl]benzoic acid (hydrochloride salt) (110 mg, 0.09150 mmol) in DMF (1.5 mL) was added HATU (42 mg, 0.1105 mmol) followed by DiPEA (80 μL, 0.4593 mmol) and the reaction mixture was stirred at room temperature for 90 min. The reaction mixture was diluted with 1:1 DMSO:MeOH with a few drops of water, filtered and purification by HPLC (1-99% ACN in water (HCl modifier)) gave (8R)-9-benzyl-13-(2,6-dimethylphenyl)-6,10-dioxa-17λ⁶-thia-3,14,16,23-tetraazatetracyclo[16.3.1.111,15.03,8]tricosa-1(22),11,13,15(23),18,20-hexaene-2,17,17-trione (38.8 mg, 74%) as a white solid. ESI-MS m/z calc. 570.19366, found 571.3 (M+1)⁺; Retention time: 1.63 minutes, LC method A.

### Example 112: Preparation of Compound 210

### Step 1; 3,3-Dimethoxypyrrolidine-2,5-dione

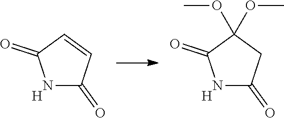

Bromine (12.409 g, 4.0 mL, 77.649 mmol) was added dropwise to a solution of pyrrole-2,5-dione (5 g, 51.508 mmol) in MeOH (200 mL) at 0° C. The reaction mixture was stirred for 16 hours at room temperature, then concentrated under reduced pressure. The crude material was dissolved in MeOH (75 mL) and added dropwise to a solution of sodium metal (4.74 g, 206.18 mmol) in MeOH (200 mL). After a further 20 hours the reaction mixture was concentrated under reduced pressure, then ethyl acetate (100 mL) was added. The mixture was neutralized by slow addition of 6M HCl, then separated between water (100 mL) and ethyl acetate (100 mL). The aqueous layer was washed with ethyl acetate (2×200 mL), then the combined organic layers were washed with brine (50 mL), dried over magnesium sulfate and concentrates under reduced pressure to afford 3,3-dimethoxypyrrolidine-2,5-dione (5.49 g, 67%) as a white solid. 1H NMR (300 MHz, METHANOL-d₄) δ 2.84 (s, 2H), 3.39 (s, 6H), 4.86 (s, 1H). ESI-MS m/z calc. 159.0532, found 182.1 (M+Na)⁺; Retention time: 0.85 minutes.

### Step 2: 3-Methoxypyrrole-2,5-dione

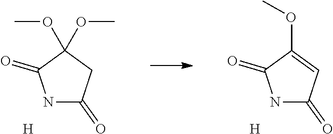

Methoxypyrrolidine-2,5-dione (5.49 g, 34.498 mmol) and p-toluenesulfonic acid hydrate (590 mg, 0.5514 mL, 3.1017 mmol) were dissolved in toluene (160 mL) and heated at reflux for 6 h. No condenser was fitted during this process, instead fresh toluene was dripped into the reaction flask at such a rate as to maintain a constant volume of solvent. Once cooled to room temperature, the reaction mixture was concentrated under reduced pressure. The residue was purified by silica gel chromatography using 0% to 40% of EtOAc in dichloromethane to afford a mixture of starting material and desired compound (˜3:1). The crude solid was dissolved in toluene (100 mL) and p-toluenesulfonic acid hydrate (260 mg, 0.2430 mL, 1.3669 mmol) was added. The reaction mixture was heated at reflux for 6 hours without condenser. Once cooled to room temperature, the reaction mixture was concentrated under reduced pressure. The residue was purified by silica gel chromatography using 0% to 60% of EtOAc in heptanes to afford 3-methoxypyrrole-2,5-dione (3.43 g, 78%) as yellow solid. ¹H NMR (300 MHz, CDCl₃) δ 3.94 (s, 3H), 5.43 (s, 1H), 7.20 (br. s., 1H). ESI-MS m/z calc. 127.0269, found (no ionization); Retention time: 0.57 minutes, LC method K.

### Step 3: 5-Benzyl-3a-methoxy-6,6a-dihydro-4H-pyrrolo[3,4-c]pyrrole-1,3-dione

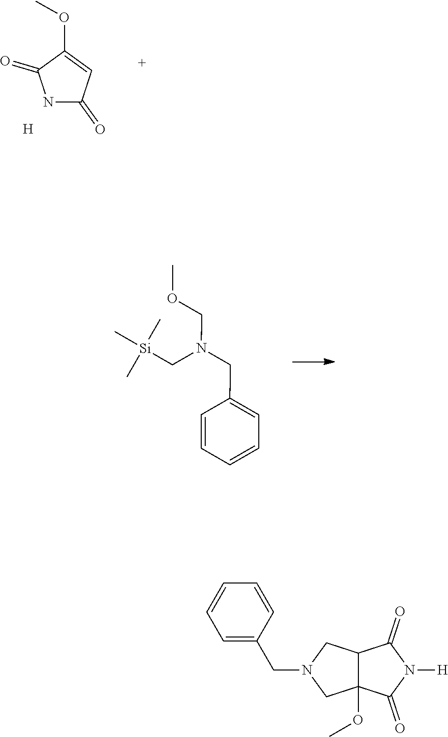

A solution of N-(methoxymethyl)-1-phenyl-N-(trimethylsilylmethyl)methanamine (10.208 g, 11 mL, 42.997 mmol) in dichloromethane (90 mL) was slowly added to a cold solution of 3-methoxypyrrole-2,5-dione (3.43 g, 26.987 mmol) and trifluoroacetic acid (370.00 mg, 0.25 mL, 3.2450 mmol) in dichloromethane (210 mL) keeping the internal reaction temperature below 2° C. The reaction mixture was allowed to warm to room temperature and stirred overnight. The reaction mixture was cooled to 0° C. and a solution of N-(methoxymethyl)-1-phenyl-N-(trimethylsilylmethyl)methanamine (3.2480 g, 3.5 mL, 13.681 mmol) in dichloromethane (5 mL) was added dropwise. After the addition, the reaction mixture was warmed to room temperature and stirred for 3 hours. The reaction mixture was washed with saturated aqueous sodium bicarbonate (100 mL). The organic layer was dried over magnesium sulfate and concentrated under reduced pressure. The residue was purified by silica gel chromatography using 0% to 30% of EtOAc in heptanes to afford 5-benzyl-3a-methoxy-6,6a-dihydro-4H-pyrrolo[3,4-c]pyrrole-1,3-dione (7.24 g, contains impurity). The compound was used for next step without any further purification. ESI-MS m/z calc. 260.1161, found 261.2 (M+1)⁺+; Retention time: 1.2 minutes (LC method C).

### Step 4: 5-Benzyl-3a-hydroxy-6,6a-dihydro-4H-pyrrolo[3,4-c]pyrrole-1,3-dione

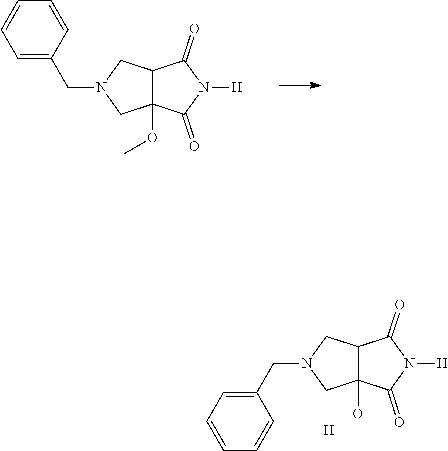

Boron tribromide (3.8 mL of 1 M, 3.8000 mmol) was slowly added to a −78° C. solution of 5-benzyl-3a-methoxy-6,6a-dihydro-4H-pyrrolo[3,4-c]pyrrole-1,3-dione (200 mg, 0.7684 mmol) in dichloromethane (10 mL). After stirring 1 hour at −78° C., the cooling bath was warmed to 0° C. for another hour and the reaction was stirred at room temperature overnight. The reaction mixture was cooled back to 0° C. and methanol was added until gas evolution was completed. The reaction mixture was concentrated under reduced pressure and co-evaporated twice with methanol to afford crude 5-benzyl-3a-hydroxy-6,6a-dihydro-4H-pyrrolo[3,4-c]pyrrole-1,3-dione (220 mg) as light orange oil. ESI-MS m/z calc. 246.1004, found 247.2 (M+1)⁺+; Retention time: 0.75 minutes, LC method K.

### Step 5: 5-Benzyl-1,2,3,4,6,6a-hexahydropyrrolo[3,4-c]pyrrol-3a-ol

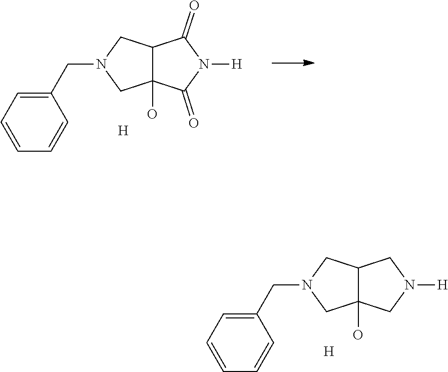

Lithium aluminum hydride (3.9 mL of 1 M in tetrahydrofuran, 3.9000 mmol) was added to a solution of 5-benzyl-3a-hydroxy-6,6a-dihydro-4H-pyrrolo[3,4-c]pyrrole-1,3-dione (190 mg, 0.7715 mmol) in tetrahydrofuran (4 mL) under nitrogen. The reaction mixture was stirred at room temperature overnight. The reaction mixture was cooled to 0° C. and quenched with water (0.15 mL), 2N aqueous sodium hydroxide (0.15 mL) and water (0.45 mL). After warming to room temperature, some magnesium sulfate was added and mixture was stirred for 30 minutes, filtrated over Celite and washed with ethyl acetate. The filtrate was concentrated under reduced pressure to afford crude 5-benzyl-1,2,3,4,6,6a-hexahydropyrrolo[3,4-c]pyrrol-3a-ol (116 mg, 69%) as clear oil. The crude material was used for next step without any further purification.

### Step 6: 3-(2-Benzyl-3a-hydroxy-3,4,6,6a-tetrahydro-1H-pyrrolo[3,4-c]pyrrole-5-carbonyl)-N-[4-chloro-6-(2,6-dimethylphenyl)pyrimidin-2-yl]benzenesulfonamide

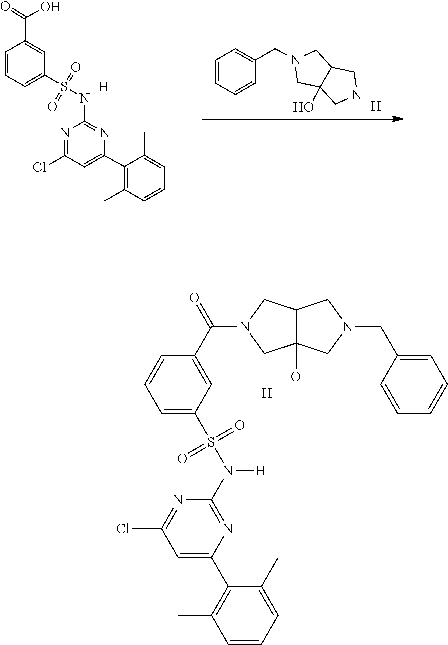

Into a solution of 3-[[4-chloro-6-(2,6-dimethylphenyl)pyrimidin-2-yl]sulfamoyl]benzoic acid (29.543 mg, 0.0707 mmol) in DMF (1 mL) was added 5-benzyl-1,2,3,4,6,6a-hexahydropyrrolo[3,4-c]pyrrol-3a-ol (hydrochloride salt) (15 mg, 0.0589 mmol), and TEA (17.880 mg, 0.0246 mL, 0.1767 mmol). HATU (33.574 mg, 0.0883 mmol) was added to the reaction mixture at 0° C. The reaction was stirred at 0° C. for 30 minutes, and then it was quenched with 10% citric acid (10 mL). The reaction mixture was extracted with DCM (3×10 mL). The combined organic layers were washed with brine (10 mL), dried over anhydrous sodium sulfate and concentrated under vacuum. The residue was purified by silica gel chromatography using 0 to 10% dichloromethane-methanol to furnish 3-(2-benzyl-3a-hydroxy-3,4,6,6a-tetrahydro-1H-pyrrolo[3,4-c]pyrrole-5-carbonyl)-N-[4-chloro-6-(2,6-dimethylphenyl)pyrimidin-2-yl]benzenesulfonamide (30 mg, 47%) as a white solid. ESI-MS m/z calc. 617.1864, found 618.2 (M+1)⁺; Retention time: 4.5 minutes, LC method S.

### Step 7: 20-Benzyl-5-(2,6-dimethylphenyl)-2-oxa-9λ⁶-thia-6,8,16,20,24-pentaazapentacyclo[14.5.1.13,7.110,14.01,18]tetracosa-3(24),4,6,10,12,14(23)-hexaene-9,9,15-trione (Compound 210)

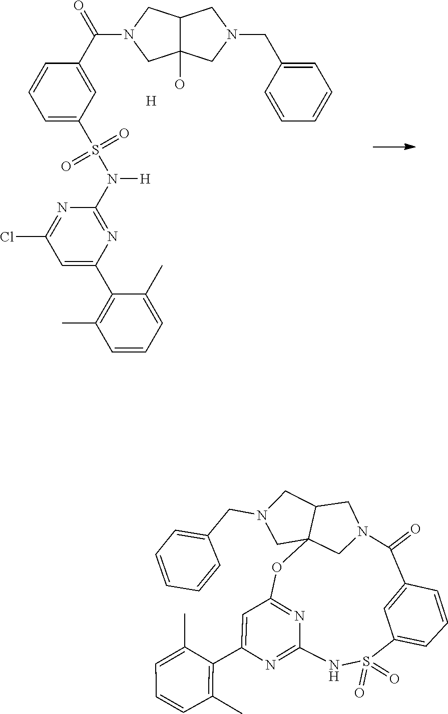

Into a solution of 3-(2-benzyl-3a-hydroxy-3,4,6,6a-tetrahydro-1H-pyrrolo[3,4-c]pyrrole-5-carbonyl)-N-[4-chloro-6-(2,6-dimethylphenyl)pyrimidin-2-yl]benzenesulfonamide (104 mg, 0.1656 mmol) in anhydrous dimethylformamide (5 mL) was added sodium hydride (66.234 mg, 60% w/w, 1.6560 mmol) at 0° C. The reaction mixture was stirred at room temperature for 24 hours. The reaction was quenched with saturated ammonium chloride (10 mL), and extracted with ethyl acetate (3×20 mL). The combined organic layers were washed with brine (3×15 mL). The organic layers were dried over anhydrous sodium sulfate and concentrated under vacuum. The residue was purified by silica gel chromatography using 0 to 5% dichloromethane-methanol to furnish 20-benzyl-5-(2,6-dimethylphenyl)-2-oxa-9λ⁶-thia-6,8,16,20,24-pentaazapentacyclo[14.5.1.13,7.110,14.01,18]tetracosa-3(24),4,6,10,12,14(23)-hexaene-9,9,15-trione (26 mg, 26%) as an off-white solid. ¹H NMR (250 MHz, DMSO-d₆) δ 8.74 (s, 1H), 7.95 (d, J=6.7 Hz, 1H), 7.70 (d, J=6.1 Hz, 2H), 7.35-7.16 (m, 6H), 7.11 (d, J=8.1 Hz, 2H), 6.73 (s, 1H), 4.51 (t, J=9.9 Hz, 1H), 3.72 (d, J=14.4 Hz, 1H), 3.61 (s, 2H), 3.08-2.68 (m, 7H), 1.93 (s, 6H). ESI-MS m/z calc. 581.2097, found 582.4 (M+1)⁺; Retention time: 1.77 minutes, LC method W.

### Example 113: Preparation of Compound 211

### Step 1: N-[4-Chloro-6-(2,6-Dimethylphenyl)pyrimidin-2-yl]-3-[(3R)-3-(hydroxymethyl)-1,2,3,4-tetrahydroisoquinoline-2-carbonyl]benzene-1-sulfonamide

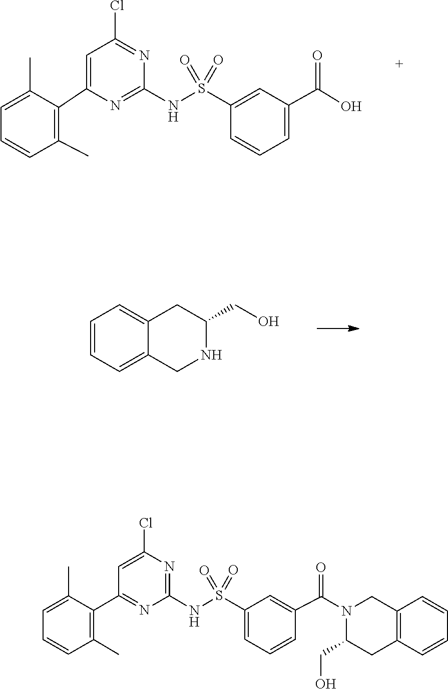 [SMILES: Cc1cccc(C)c1-c1cc(Cl)nc(NS(=O)(=O)c2cccc(C(=O)O)c2)n1 ; OC[C@H]1Cc2ccccc2CN1 ; Cc1cccc(C)c1-c1cc(Cl)nc(NS(=O)(=O)c2cccc(C(=O)N3Cc4ccccc4C[C@@H]3CO)c2)n1]

To DIC (approximately 20.02 mg, 24.84 μL, 0.1586 mmol) was added 3-[[4-chloro-6-(2,6-dimethylphenyl)pyrimidin-2-yl]sulfamoyl]benzoic acid (51 mg, 0.1220 mmol) dissolved in dichloromethane (0.5 mL) followed by solid sodium bicarbonate (approximately 51.24 mg, 0.6100 mmol). The suspension was allowed to stir at room temperature for 15 minutes. [(3R)-1,2,3,4-tetrahydroisoquinolin-3-yl]methanol (approximately 21.90 mg, 0.1342 mmol) was added after. The final reaction mixture was stirred at room temperature for 2 hours. It was then diluted with DMSO and purified by reverse phase HPLC method using a Luna C₁₈ (2) column (50×21.2 mm, 5 μm particle size) sold by Phenomenex (pn: 00B-4252-P0-AX), and a dual gradient run from 10-99% mobile phase B over 15.0 minutes. Mobile phase A=water (5 mM HCl acid modifier). Mobile phase B=acetonitrile. Flow rate=35 mL/min, injection volume=950 μL, and column temperature=25° C. The UV trace at 254 nm was used to collect fractions. N-[4-Chloro-6-(2,6-Dimethylphenyl)pyrimidin-2-yl]-3-[(3R)-3-(hydroxymethyl)-1,2,3,4-tetrahydroisoquinoline-2-carbonyl]benzene-1-sulfonamide (18.7 mg, 27%) was obtained.

### Step 2: (12R)-17-(2,6-Dimethylphenyl)-14-oxa-21λ⁶-thia-3,18,20,27-tetraazapentacyclo[20.3.1.115,19.03,12.05,10]heptacosa-1(26),5(10),6,8,15(27),16,18,22,24-nonaene-2,21,21-trione (Compound 211)

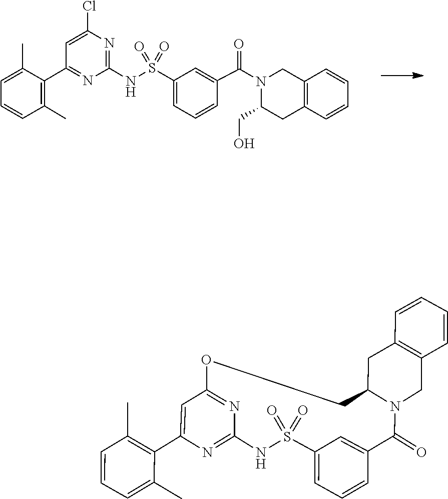 [SMILES: Cc1cccc(C)c1-c1cc(Cl)nc(NS(=O)(=O)c2cccc(C(=O)N3Cc4ccccc4C[C@@H]3CO)c2)n1 ; Cc1cccc(C)c1-c1cc2nc(n1)NS(=O)(=O)c1cccc(c1)C(=O)N1Cc3ccccc3C[C@@H]1CO2]

A solution of N-[4-chloro-6-(2,6-Dimethylphenyl)pyrimidin-2-yl]-3-[(3R)-3-(hydroxymethyl)-1,2,3,4-tetrahydroisoquinoline-2-carbonyl]benzene-1-sulfonamide (17.6 mg, 0.03126 mmol) in 1-methyl-pyrrolidin-2-one (5 mL) was added to sodium hydride (approximately 12.50 mg, 13.89 μL, 0.3126 mmol) (60 wt % dispersion in mineral oil). The reaction mixture was sealed and allowed to stir at 70° C. for 2 hours. The sample was purified by reverse phase HPLC method using a Luna C₁₈ (2) column (50×21.2 mm, 5 μm particle size) sold by Phenomenex (pn: 00B-4252-P0-AX), and a dual gradient run from 10-99% mobile phase B over 15.0 minutes. Mobile phase A=water (5 mM acid modifier). Mobile phase B=acetonitrile. Flow rate=35 mL/min, injection volume=950 μL, and column temperature=25° C. The UV trace at 254 nm was used to collect fractions. (12R)-17-(2,6-dimethylphenyl)-14-oxa-21λ⁶-thia-3,18,20,27-tetraazapentacyclo[20.3.1.115,19.03,12.05,10]heptacosa-1(26),5(10),6,8,15(27),16,18,22,24-nonaene-2,21,21-trione (6.8 mg, 41%) was obtained. ESI-MS m/z calc. 526.1675, found 527.3 (M+1)⁺; Retention time: 1.73 minutes; LC method A.

### Example 114: Preparation of Compound 212

### Step 1: N-[4-Chloro-6-(2,6-dimethylphenyl)pyrimidin-2-yl]-3-[(3S)-3-(hydroxymethyl)-1,2,3,4-tetrahydroisoquinoline-2-carbonyl]benzene-1-sulfonamide

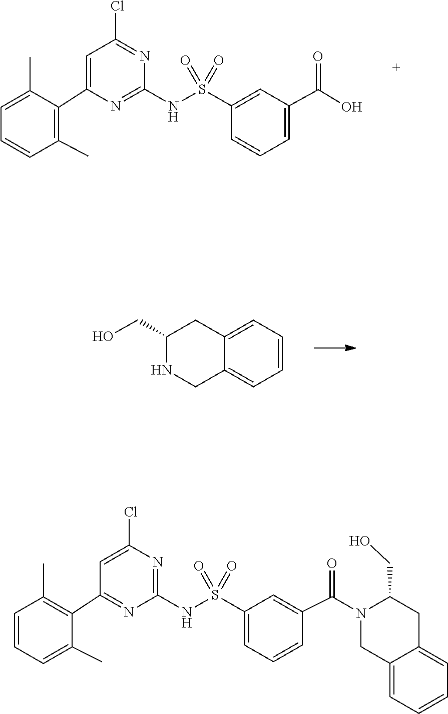 [SMILES: Cc1cccc(C)c1-c1cc(Cl)nc(NS(=O)(=O)c2cccc(C(=O)O)c2)n1 ; OC[C@@H]1Cc2ccccc2CN1 ; Cc1cccc(C)c1-c1cc(Cl)nc(NS(=O)(=O)c2cccc(C(=O)N3Cc4ccccc4C[C@H]3CO)c2)n1]

3-[[4-chloro-6-(2,6-dimethylphenyl)pyrimidin-2-yl]sulfamoyl]benzoic acid (60 mg, 0.1436 mmol) was dissolved in dichloromethane and added to N,N′-diisopropylcarbodiimide. The mixture was allowed to stir at room temperature for 30 minutes. The obtained suspension was then added to [(3S)-1,2,3,4-tetrahydroisoquinolin-3-yl]methanol (approximately 25.79 mg, 0.1580 mmol). Solid sodium bicarbonate was added last. The reaction mixture was allowed to stir overnight at room temperature. The product purified using a reverse phase HPLC method using a Luna C₁₈ (2) column (50×21.2 mm, 5 μm particle size) sold by Phenomenex (pn: 00B-4252-P0-AX), and a dual gradient run from 10-99% mobile phase B over 15.0 minutes. Mobile phase A=water (5 mM acid modifier). Mobile phase B=acetonitrile. Flow rate=35 mL/min, injection volume=950 μL, and column temperature=25° C. The UV trace at 254 nm was used to collect fractions. N-[4-Chloro-6-(2,6-dimethylphenyl)pyrimidin-2-yl]-3-[(3S)-3-(hydroxymethyl)-1,2,3,4-tetrahydroisoquinoline-2-carbonyl]benzene-1-sulfonamide (26.2 mg, 32%) was obtained. ESI-MS m/z calc. 562.14417, found 563.3 (M+1)⁺; Retention time: 1.75 minutes; LC method A.

### Step 2: (12S)-17-(2,6-Dimethylphenyl)-14-oxa-21λ⁶-thia-3,18,20,27-tetraazapentacyclo[20.3.1.115,19.03,12.05,10]heptacosa-1(26),5(10),6,8,15(27),16,18,22,24-nonaene-2,21,21-trione (Compound 212)

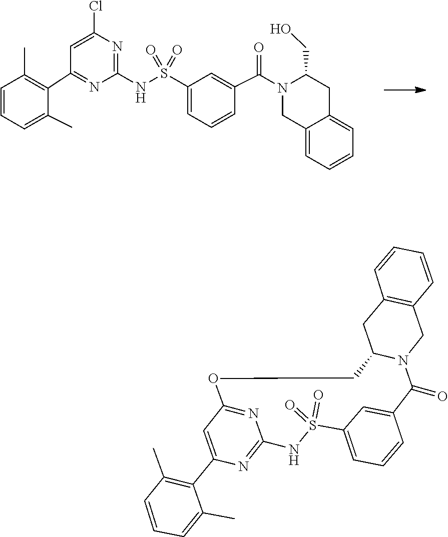 [SMILES: Cc1cccc(C)c1-c1cc(Cl)nc(NS(=O)(=O)c2cccc(C(=O)N3Cc4ccccc4C[C@H]3CO)c2)n1 ; Cc1cccc(C)c1-c1cc2nc(n1)NS(=O)(=O)c1cccc(c1)C(=O)N1Cc3ccccc3C[C@H]1CO2]

A solution of N-[4-Chloro-6-(2,6-dimethylphenyl)pyrimidin-2-yl]-3-[(3S)-3-(hydroxymethyl)-1,2,3,4-tetrahydroisoquinoline-2-carbonyl]benzene-1-sulfonamide (26.2 mg, 0.04653 mmol) in 1-methyl-pyrrolidin-2-one (5 mL) was added to sodium hydride (approximately 18.61 mg, 20.68 μL, 0.4653 mmol) (60 wt % dispersion in mineral oil). The reaction mixture was sealed and allowed to stir at 70° C. for 2 hours. The product was purified by reverse phase HPLC using a Luna C₁₈ (2) column (50×21.2 mm, 5 μm particle size) sold by Phenomenex (pn: 00B-4252-P0-AX), and a dual gradient run from 10-99% mobile phase B over 15.0 minutes. Mobile phase A=water (5 mM acid modifier). Mobile phase B=acetonitrile. Flow rate=35 mL/min, injection volume=950 μL, and column temperature=25° C. The UV trace at 254 nm was used to collect fractions. (12S)-17-(2,6-dimethylphenyl)-14-oxa-21λ⁶-thia-3,18,20,27-tetraazapentacyclo[20.3.1.115,19.03,12.05,10]heptacosa-1(26),5(10),6,8,15(27),16,18,22,24-nonaene-2,21,21-trione (8 mg, 33%) was obtained. ESI-MS m/z calc. 526.1675, found 527.3 (M+1)⁺; Retention time: 1.73 minutes; LC method A.

### Example 115: Preparation of Compound 213

### Step 1: 3-{[4-(2,6-Dimethylphenyl)-6-[(3S)-pyrrolidin-3-yloxy]pyrimidin-2-yl]sulfamoyl}benzoic acid

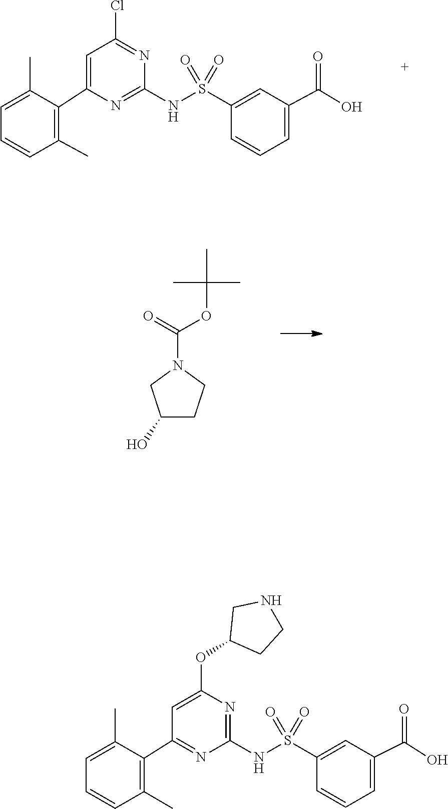 [SMILES: Cc1cccc(C)c1-c1cc(Cl)nc(NS(=O)(=O)c2cccc(C(=O)O)c2)n1 ; CC(C)(C)OC(=O)N1CC[C@H](O)C1 ; Cc1cccc(C)c1-c1cc(O[C@H]2CCNC2)nc(NS(=O)(=O)c2cccc(C(=O)O)c2)n1]

3-[[4-Chloro-6-(2,6-dimethylphenyl)pyrimidin-2-yl]sulfamoyl]benzoic acid (50 mg, 0.1197 mmol), tert-butyl (3S)-3-hydroxypyrrolidine-1-carboxylate (approximately 44.82 mg, 0.2394 mmol), and sodium tert-butoxide (approximately 46.01 mg, 0.4788 mmol) were combined in THE (1 mL) and stirred at room temperature for 16 h. The reaction mixture was partitioned between ethyl acetate and a 1M solution of HCl. The organics were separated, washed with brine, dried over sodium sulfate and evaporated. The crude material was dissolved in 4M HCl in dioxane (2 mL of 4 M, 8.000 mmol) and stirred for 30 min. The reaction was evaporated and the resulting material was purified by LC/MS utilizing a gradient of 1-99% acetonitrile in 5 mM aqueous HCl to yield 3-{[4-(2,6-dimethylphenyl)-6-[(3S)-pyrrolidin-3-yloxy]pyrimidin-2-yl]sulfamoyl}benzoic acid (hydrochloride salt) (34.5 mg, 57%). ESI-MS m/z calc. 468.14673, found 469.3 (M+1)⁺; Retention time: 0.36 minutes; (LC method D).

### Step 2: (3S)-17-(2,6-Dimethylphenyl)-2-oxa-13λ⁶-thia-6,14,16,19-tetraazatetracyclo[13.3.1.13,6.18,12]henicosa-1(19),8(20),9,11,15,17-hexaene-7,13,13-trione (Compound 213)

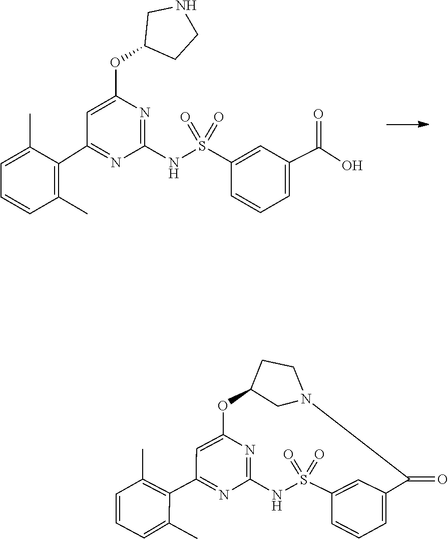 [SMILES: Cc1cccc(C)c1-c1cc(O[C@H]2CCNC2)nc(NS(=O)(=O)c2cccc(C(=O)O)c2)n1 ; Cc1cccc(C)c1-c1cc2nc(n1)NS(=O)(=O)c1cccc(c1)C(=O)N1CC[C@@H](C1)O2]

3-{[4-(2,6-Dimethylphenyl)-6-[(3S)-pyrrolidin-3-yloxy]pyrimidin-2-yl]sulfamoyl}benzoic acid (hydrochloride salt) (27.1 mg, 0.05366 mmol), HATU (approximately 20.40 mg, 0.05366 mmol), and triethylamine (approximately 16.29 mg, 22.44 μL, 0.1610 mmol) were combined in DMF (1 mL) and stirred at room temperature for 1 h. The reaction mixture was filtered and purified by LC/MS utilizing a gradient of 1-99% acetonitrile in 5 mM aqueous HCl to yield (3S)-17-(2,6-dimethylphenyl)-2-oxa-13λ⁶-thia-6,14,16,19-tetraazatetracyclo[13.3.1.13,6.18,12]henicosa-1(19),8(20),9,11,15,17-hexaene-7,13,13-trione (4.8 mg, 20%). ESI-MS m/z calc. 450.13617, found 451.2 (M+1)⁺; Retention time: 1.26 minutes; (LC method A).

### Example 116: Preparation of Compound 214

### Step 1: 3-{[4-(2,6-dimethylphenyl)-6-[(3R)-pyrrolidin-3-yloxy]pyrimidin-2-yl]sulfamoyl}benzoic acid

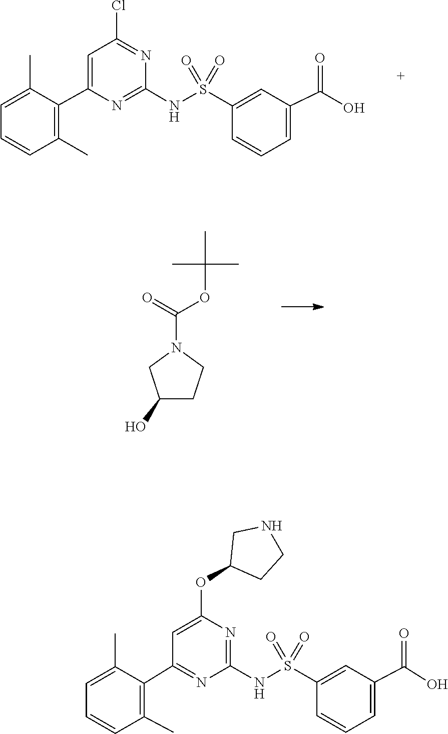 [SMILES: Cc1cccc(C)c1-c1cc(Cl)nc(NS(=O)(=O)c2cccc(C(=O)O)c2)n1 ; CC(C)(C)OC(=O)N1CC[C@@H](O)C1 ; Cc1cccc(C)c1-c1cc(O[C@@H]2CCNC2)nc(NS(=O)(=O)c2cccc(C(=O)O)c2)n1]

3-[[4-Chloro-6-(2,6-dimethylphenyl)pyrimidin-2-yl]sulfamoyl]benzoic acid (50 mg, 0.1197 mmol), tert-butyl (3R)-3-hydroxypyrrolidine-1-carboxylate (approximately 44.82 mg, 0.2394 mmol), and sodium tert-butoxide (approximately 46.01 mg, 0.4788 mmol) were combined in THE (1 mL) and stirred at room temperature for 16 h. The reaction mixture was partitioned between ethyl acetate and a 1M solution of HCl. The organics were separated, washed with brine, dried over sodium sulfate and evaporated. The crude material was dissolved in 4M HCl in dioxane (2 mL of 4 M, 8.000 mmol) and stirred for 30 min. The reaction was evaporated and the resulting material was purified by LC/MS utilizing a gradient of 1-99% acetonitrile in 5 mM aqueous HCl to yield 3-{[4-(2,6-dimethylphenyl)-6-[(3R)-pyrrolidin-3-yloxy]pyrimidin-2-yl]sulfamoyl}benzoic acid (hydrochloride salt) (35.5 mg, 59%). ESI-MS m/z calc. 468.14673, found 469.3 (M+1)⁺; Retention time: 0.36 minutes; (LC method D).

### Step 2: (3R)-17-(2,6-Dimethylphenyl)-2-oxa-13λ⁶-thia-6,14,16,19-tetraazatetracyclo[13.3.1.13,6.18,12]henicosa-1(19),8(20),9,11,15,17-hexaene-7,13,13-trione (Compound 214)

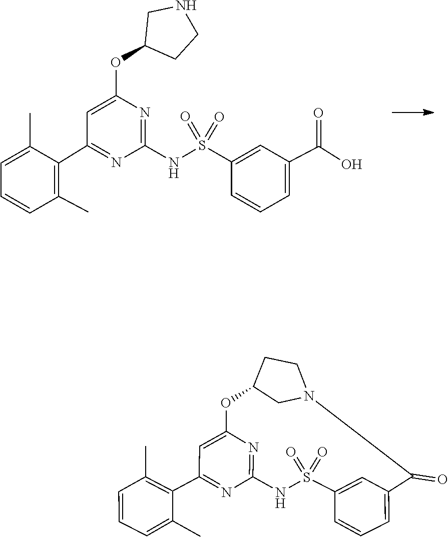 [SMILES: Cc1cccc(C)c1-c1cc(O[C@@H]2CCNC2)nc(NS(=O)(=O)c2cccc(C(=O)O)c2)n1 ; Cc1cccc(C)c1-c1cc2nc(n1)NS(=O)(=O)c1cccc(c1)C(=O)N1CC[C@H](C1)O2]

3-{[4-(2,6-Dimethylphenyl)-6-[(3R)-pyrrolidin-3-yloxy]pyrimidin-2-yl]sulfamoyl}benzoic acid (hydrochloride salt) (28.7 mg, 0.05683 mmol), HATU (approximately 21.61 mg, 0.05683 mmol), and triethylamine (approximately 17.25 mg, 23.76 μL, 0.1705 mmol) were combined in DMF (1 mL) and stirred at room temperature for 1 h. The reaction mixture was filtered and purified by LC/MS utilizing a gradient of 1-99% acetonitrile in 5 mM aqueous HCl to yield (3R)-17-(2,6-Dimethylphenyl)-2-oxa-13λ⁶-thia-6,14,16,19-tetraazatetracyclo[13.3.1.13,6.18,12]henicosa-1(19),8(20),9,11,15,17-hexaene-7,13,13-trione (4.5 mg, 17%). ESI-MS m/z calc. 450.13617, found 451.3 (M+1)⁺; Retention time: 1.26 minutes; LC method A.

### Example 117: Preparation of Compound 215, Compound 216, and Compound 217

### Step 1: 2-Allyl-benzoic acid methyl ester

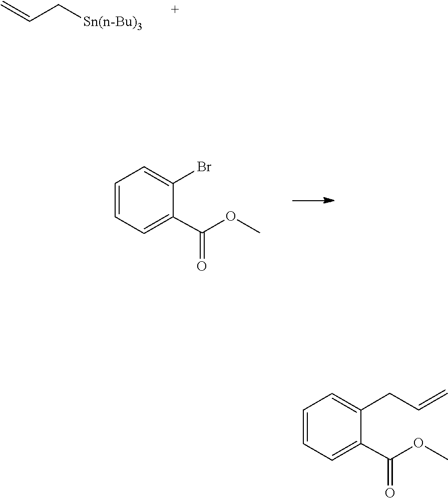

2-Bromo-benzoic acid methyl ester (23.0 g, 0.107 mol), allyl-tributyl-stannane (42.5 g, 0.128 mmol) and tetrakis(triphenylphosphine)palladium(0) (2.47 g, 2.14 mmol) were dissolved in anhydrous benzene (40 mL) in a sealed tube. The reaction solution was purged with argon for 5 minutes, then heated at 100° C. for 20 hours. The reaction solution was filtered through a pad of Si gel and washed with hexane. The filtrate was concentrated, and the residue was distilled under the reduced pressure (85 to 87° C./3 mmHg) to afford colorless liquid which was contaminated with 14% of the stannane impurity. The impure product was further purified by silica gel column chromatography using 0-50% hexanes-dichloromethane to afford 2-allyl-benzoic acid methyl ester (12.3 g, 65%) as a colorless liquid. ¹H NMR (250 MHz, CDCl₃) δ (ppm): 7.90 (m, 1H), 7.43 (m, 1H), 7.26 (m, 2H), 6.00 (m, 1H), 5.06-4.98 (m, 2H), 3.89 (s, 3H), 3.76 (d, J=6.3 Hz, 2H). ESI-MS m/z calc. 176.08, found 177.8 (M+1)⁺. Retention time: 5.13 minutes.

### Step 2: 3-Iodomethyl-isochroman-1-one

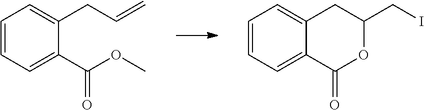

To a solution of 2-allyl-benzoic acid methyl ester (11.9 g, 67.3 mmol) in acetonitrile (70 mL) was added iodine (34.2 g, 0.135 mol) and the reaction solution was stirred at ambient temperature for 1 hour. The mixture was diluted with ethyl acetate (900 mL). The organic solution was washed with saturated sodium bisulfite solution, dried over magnesium sulfate and concentrated. The residue obtained was purified by silica gel chromatography using 0-30% ethyl acetate in hexanes to afford 3-iodomethyl-isochroman-1-one (17.5 g, 87%) as a pale-yellow liquid. ¹H NMR (250 MHz, CDCl₃) δ (ppm): 8.10 (d, J=7.5 Hz, 1H), 7.58 (t, J=7.5 Hz, 1H), 7.42 (t, J=7.5 Hz, 1H), 7.29 (d, J=7.5 Hz, 1H), 4.56 (m, 1H), 3.45 (m, 2H), 3.19 (m, 2H). ESI-MS m/z calc. 287.98, found 289.0 (M+1)⁺. Retention time: 4.32 minutes.

### Step 3: 4-Hydroxy-2,3,4,5-tetrahydro-benzo[c]azepin-1-one

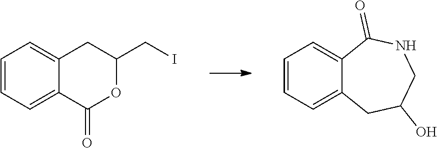 [SMILES: O=C1OC(CI)Cc2ccccc21 ; O=C1NCC(O)Cc2ccccc21]

To a solution of 7 N ammonia in methanol (1170 mL) was added a solution of 3-iodomethyl-isochroman-1-one (16.9 g, 58.7 mmol) in anhydrous methanol (120 mL) dropwise at 0° C. The resulting solution was stirred at ambient temperature for 67 hours. All solvent was removed under reduced pressure. The residue obtained was dissolved in 7 N ammonia in methanol (10 mL) and silica gel was added. The solvent was removed, and the silica gel subjected to column chromatography using 0-13% dichloromethane-methanol (1% ammonia) to afford 4-hydroxy-2,3,4,5-tetrahydro-benzo[c]azepin-1-one (5.72 g, 55%) as a white solid. ¹H NMR (250 MHz, DMSO) δ (ppm): 8.09 (t, J=5.5 Hz, 1H), 7.52 (dd, J=1.5, 7.5 Hz, 1H), 7.38 (m, 2H), 7.24 (d, J=7.5 Hz, 1H), 5.06 (d, J=4.0 Hz, 1H), 4.11 (m, 1H), 2.96 (m, 2H), 2.62 (m, 2H). ESI-MS m/z calc. 177.08, found 178.1 (M+1)⁺. Retention time: 1.50 minutes.

### Step 4: 2,3,4,5-Tetrahydro-1H-benzo[c]azepin-4-ol

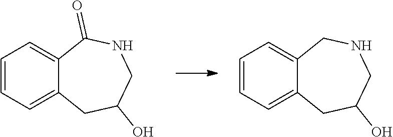 [SMILES: O=C1NCC(O)Cc2ccccc21 ; OC1CNCc2ccccc2C1]

4-Hydroxy-2,3,4,5-tetrahydro-benzo[c]azepin-1-one (5.72 g, 32.3 mmol) was dissolved in a 1 N borane-tetrahydrofuran solution (200 mL). The resulting solution was heated at 60° C. for 63 hours, then cooled to 0° C. 6 N Aqueous hydrogen chloride solution was added dropwise until no further bubbling was observed (pH=2). The solvent was removed under the reduced pressure and the aqueous solution was washed with diethyl ether (2×100 mL), then basified with 6 N aqueous sodium hydroxide solution to pH=10. The aqueous layer was extracted with 2-methyl tetrahydrofuran (5×150 mL) and the combined organic layer was dried over sodium sulfate and concentrated to afford 2,3,4,5-tetrahydro-1H-benzo[c]azepin-4-ol (5.20 g, 99%) as a white solid, which was directly used in next step without further purification. ESI-MS m/z calc. 163.10, found 163.7 (M+1)⁺. Retention time: 1.17 minutes.

### Step 5: 4-Hydroxy-1,3,4,5-tetrahydro-benzo[c]azepine-2-carboxylic acid tert-butyl ester

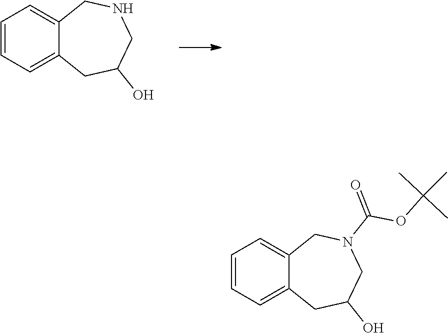 [SMILES: OC1CNCc2ccccc2C1 ; CC(C)(C)OC(=O)N1Cc2ccccc2CC(O)C1]

To a solution of 2,3,4,5-tetrahydro-1H-benzo[c]azepin-4-ol (5.20 g, 31.88 mmol) in anhydrous dichloromethane (320 mL) (cloudy solution) was added triethyl amine (3.87 g, 38.26 mmol) followed by di-tert-butyl dicarbonate (6.61 g, 30.29 mmol). The resulting solution was stirred at ambient temperature for 30 minutes and then diluted with dichloromethane (500 mL). The organic solution was washed with brine, dried over sodium sulfate and concentrated. The residue was purified by silica gel column chromatography using 0-60% hexanes-ethyl acetate to afford 4-hydroxy-1,3,4,5-tetrahydro-benzo[c]azepine-2-carboxylic acid tert-butyl ester (6.93 g, 83%) as a white solid. ¹H NMR (250 MHz, CDCl₃) δ (ppm): 4.45 (d, J=15.0 Hz, 1H), 4.34 (d, J=15.0 Hz, 1H), 3.98 (m, 1H), 3.75 (m, 2H), 3.11 (d, J=5.8 Hz, 1H), 1.40 (s, 9H). ESI-MS m/z calc. 263.17, found 264.2 (M+1)⁺. Retention time: 2.09 minutes.

### Step 6: 12-(2,6-Dimethylphenyl)-15-oxa-8λ⁶-thia-1,9,11,26-tetraazapentacyclo[14.8.1.13,7.110,14.018,23]heptacosa-3(27),4,6,10,12,14(26),18,20,22-nonaene-2,8,8-trione, racemic mixture (Compound 215), 12-(2,6-dimethylphenyl)-15-oxa-8λ⁶-thia-1,9,11,26-tetraazapentacyclo[14.8.1.13,7.110,14.018,23]heptacosa-3(27),4,6,10,12,14(26),18,20,22-nonaene-2,8,8-trione, enantiomer 1 (Compound 216), and 12-(2,6-dimethylphenyl)-15-oxa-8λ⁶-thia-1,9,11,26-tetraazapentacyclo[14.8.1.13,7.110,14.018,23]heptacosa-3(27),4,6,10,12,14(26),18,20,22-nonaene-2,8,8-trione, enantiomer 2 (Compound 217)

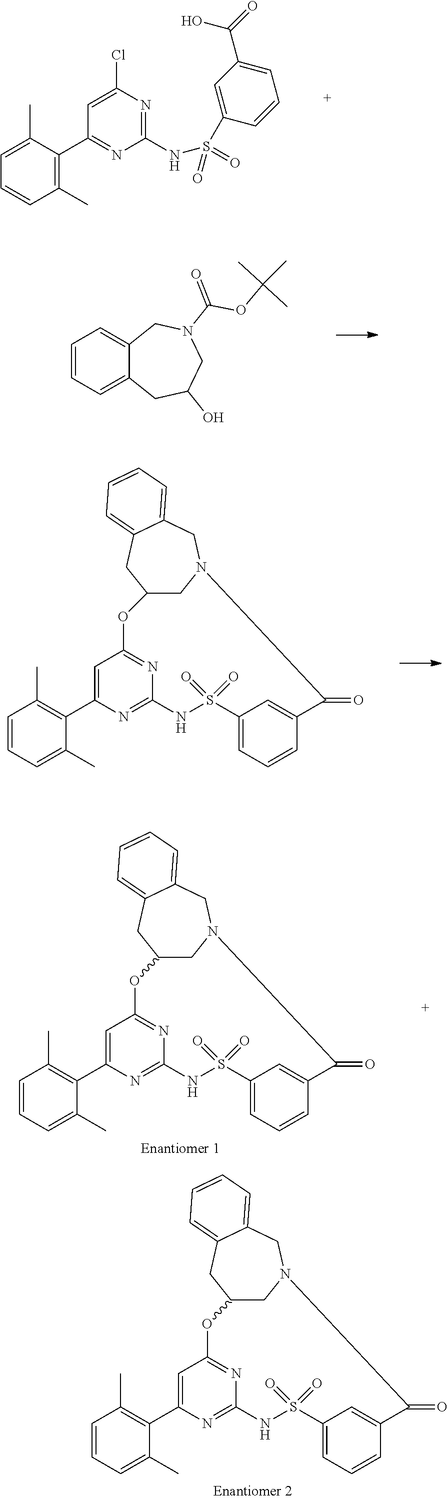 [SMILES: Cc1cccc(C)c1-c1cc(Cl)nc(NS(=O)(=O)c2cccc(C(=O)O)c2)n1 ; CC(C)(C)OC(=O)N1Cc2ccccc2CC(O)C1 ; Cc1cccc(C)c1-c1cc2nc(n1)NS(=O)(=O)c1cccc(c1)C(=O)N1Cc3ccccc3CC(C1)O2 ; Cc1cccc(C)c1-c1cc2nc(n1)NS(=O)(=O)c1cccc(c1)C(=O)N1Cc3ccccc3CC(C1)O2 ; Cc1cccc(C)c1-c1cc2nc(n1)NS(=O)(=O)c1cccc(c1)C(=O)N1Cc3ccccc3CC(C1)O2]

3-[[4-chloro-6-(2,6-dimethylphenyl)pyrimidin-2-yl]sulfamoyl]benzoic acid (125 mg, 0.2991 mmol), tert-butyl 4-hydroxy-1,3,4,5-tetrahydro-2-benzazepine-2-carboxylate (103 mg, 0.3911 mmol), and NaH (70 mg, 1.750 mmol) were combined in anhydrous NMP (1 mL), and stirred for 1 hour at room temperature. The reaction mixture was then quenched with 0.2 mL acetic acid, diluted with methanol, filtered, and purified by reverse phase HPLC (1-70% ACN HCl modifier, 15 min run), to give 3-[[4-[(2-tert-butoxycarbonyl-1,3,4,5-tetrahydro-2-benzazepin-4-yl)oxy]-6-(2,6-dimethylphenyl)pyrimidin-2-yl]sulfamoyl]benzoic acid (95 mg, 49%) ESI-MS m/z calc. 644.23047, found 645.4 (M+1)⁺; Retention time: 0.71 minutes (LC method D).

The product was dissolved in dichloromethane (2 mL), and HCl (2 mL of 4 M, 8.000 mmol) in dioxane was added. After stirring at room temperature for 1 hour, the reaction mixture was concentrated under vacuum to give 3-[[4-(2,6-dimethylphenyl)-6-(2,3,4,5-tetrahydro-1H-2-benzazepin-4-yloxy)pyrimidin-2-yl]sulfamoyl]benzoic acid (hydrochloride salt) (88 mg, 51%) ESI-MS m/z calc. 544.17804, found 545.4 (M+1)⁺; Retention time: 0.42 minutes (LC method D).

The product was combined with HATU (75 mg, 0.1972 mmol) in dichloromethane (10 mL), and DIPEA (160 μL, 0.9186 mmol) was added. The reaction mixture was stirred for 2 hours at room temperature then concentrated by rotary evaporation. The resulting crude material was dissolved in 1:1 DMSO/methanol, filtered, and purified by reverse phase HPLC (1-70% ACN in water with HCl 15 min run), to give racemic 12-(2,6-dimethylphenyl)-15-oxa-8λ⁶-thia-1,9,11,26-tetraazapentacyclo[14.8.1.13,7.110,14.018,23]heptacosa-3(27),4,6,10,12,14(26),18,20,22-nonaene-2,8,8-trione (45 mg, 29%) ESI-MS m/z calc. 526.1675, found 527.3 (M+1)⁺; Retention time: 1.65 minutes (LC method A). This material was subjected to chiral SFC using a ChiralCel OJ-H (250×10 mm, 5 m) column, a mobile phase comprised of 18% MeOH (no modifier) and 82% CO₂, with a flow of 10 mL/min, at a concentration of 16 mg/mL in MeOH:DMSO (88:12), and with an injection volume of 70 μL, at a pressure of 100 bar and utilizing a 220 nm wavelength to give two enantiomers: Enantiomer 1, peak 1 12-(2,6-dimethylphenyl)-15-oxa-8λ⁶-thia-1,9,11,26-tetraazapentacyclo[14.8.1.13,7.110,14.018,23]heptacosa-3(27),4,6,10,12,14(26),18,20,22-nonaene-2,8,8-trione (5.1 mg, 3%) ESI-MS m/z calc. 526.1675, found 527.4 (M+1)⁺; Retention time: 1.64 minutes (LC method A); and enantiomer 2, peak 2 12-(2,6-dimethylphenyl)-15-oxa-8λ⁶-thia-1,9,11,26-tetraazapentacyclo[14.8.1.13,7.110,14.018,23]heptacosa-3(27),4,6,10,12,14(26),18,20,22-nonaene-2,8,8-trione (4.4 mg, 3%) ESI-MS m/z calc. 526.1675, found 527.4 (M+1)⁺; Retention time: 1.64 minutes (LC method A).

### Example 118: Preparation of Compound 218 and Compound 219

### Step 1: (3-tert-Butylphenyl)methanamine

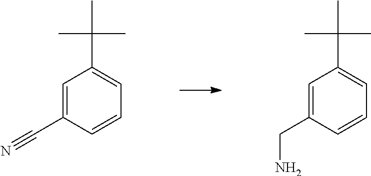

To a solution of 3-tert-butylbenzonitrile (4.58 g, 28.764 mmol) in MeOH (135 mL) was added Raney nickel 2400 (7.2 g, 36.801 mmol) with water. The vessel was evacuated and filled with hydrogen (three cycles). NH₃ (13 mL of 28% w/v, 213.73 mmol) in water was added by syringe. The mixture was stirred at rt under hydrogen (balloon) for 18 h. The mixture was decanted and filtered through diatomaceous earth and washed with MeOH. The filtrate was concentrated. The residue was extracted with DCM and dried with sodium sulfate. Flash chromatography on flash chromatography (120 g silica gel, DCM (1% NH₃)/MeOH 0-8%) afforded (3-tert-butylphenyl)methanamine (4.51 g, 96%) as pale-yellow oil. ESI-MS m/z calc. 163.1361, found 164.2 (M+1)⁺; Retention time: 1.45 minutes. ESI-MS m/z calc. 163.1361, found 164.2 (M+1)⁺; Retention time: 1.45 minutes; LC method K.

### Step 2: Ethyl 7-tert-butyl-3-methyl-isoquinoline-4-carboxylate

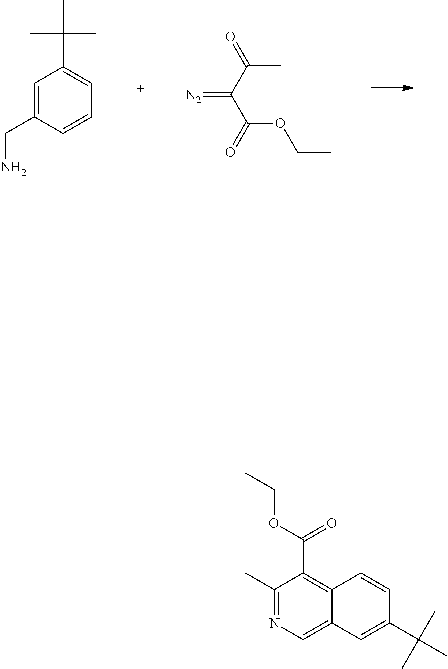

A 1 L flask with a condenser was charged with (3-tert-butylphenyl)methanamine (4.75 g, 29.095 mmol), ethyl 2-diazo-3-oxo-butanoate (6.21 g, 35.795 mmol), 2,2,2-trifluoroethanol (250 mL), pentamethylcyclopentadienylrhodium(III) chloride dimer (452 mg, 0.7313 mmol) and silver acetate (974 mg, 5.8354 mmol). The mixture opened to air was stirred at 72° C. overnight. The mixture was diluted with EtOAc, filtered through diatomaceous earth and washed with EtOAc. The filtrate was concentrated on silica gel (40 g) and purified by flash chromatography (120 g silica gel, heptanes/EtOAc 0-20%) to afford ethyl 7-tert-butyl-3-methyl-isoquinoline-4-carboxylate (5.55 g, 70%) as pale-yellow oil. ¹H NMR (300 MHz, CDCl₃) δ 9.18 (s, 1H), 7.93-7.74 (m, 3H), 4.54 (q, J=7.0 Hz, 2H), 2.73 (s, 3H), 1.47 (t, J=7.2 Hz, 3H), 1.41 (s, 9H). ESI-MS m/z calc. 271.15723, found 272.2 (M+1)⁺; Retention time: 2.06 minutes; LC method K.

### Step 3: Ethyl 7-tert-butyl-3-methyl-1,2,3,4-tetrahydroisoquinoline-4-carboxylate

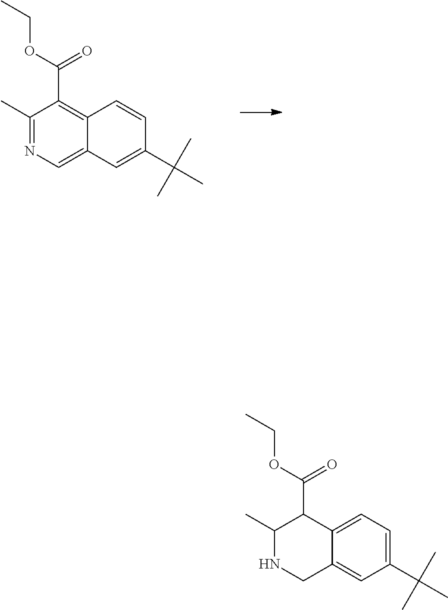 [SMILES: CCOC(=O)c1c(C)ncc2cc(C(C)(C)C)ccc12 ; CCOC(=O)C1c2ccc(C(C)(C)C)cc2CNC1C]

To a solution of ethyl 7-tert-butyl-3-methyl-isoquinoline-4-carboxylate (5.19 g, 19.126 mmol) in MeOH (90 mL) was added nickel(II) chloride hexahydrate (9.1 g, 38.285 mmol). The mixture was stirred at rt for 10 min and cooled with an ice-water cooling bath. sodium borohydride (14.5 g, 383.27 mmol) was added in portions over 1 h, keeping the inner temperature at 28-33° C. The mixture was stirred at 25° C. for 10 min. The mixture was concentrated to remove MeOH. The residue was treated with 3 N aq. HCl (180 mL) at 0° C. pH=1. The mixture was stirred at rt for 30 min and neutralized with 28% aq. NH₃ (200 mL) at 0° C. The mixture was extracted with DCM, dried over sodium sulfate. The mixture was concentrated, and the residue was purified by flash chromatography (120 g silica, DCM/MeOH 0-8%) to afford ethyl 7-tert-butyl-3-methyl-1,2,3,4-tetrahydroisoquinoline-4-carboxylate (2.49 g, 47%) as light yellow oil. ¹H NMR (300 MHz, CDCl₃) δ 7.22-7.04 (m, 3H), 4.28-4.11 (m, 2H), 4.08 (s, 2H), 3.57 (d, J=4.1 Hz, 1H), 3.12 (qd, J=6.8, 4.1 Hz, 1H), 2.36-2.00 (m, 1H), 1.29 (s, 15H). ESI-MS m/z calc. 275.18854, found 276.2 (M+1)⁺; Retention time: 1.62 minutes; LC method K.

### Step 4: (7-tert-Butyl-3-methyl-1,2,3,4-tetrahydroisoquinolin-4-yl)methanol diastereomer 1 and 2

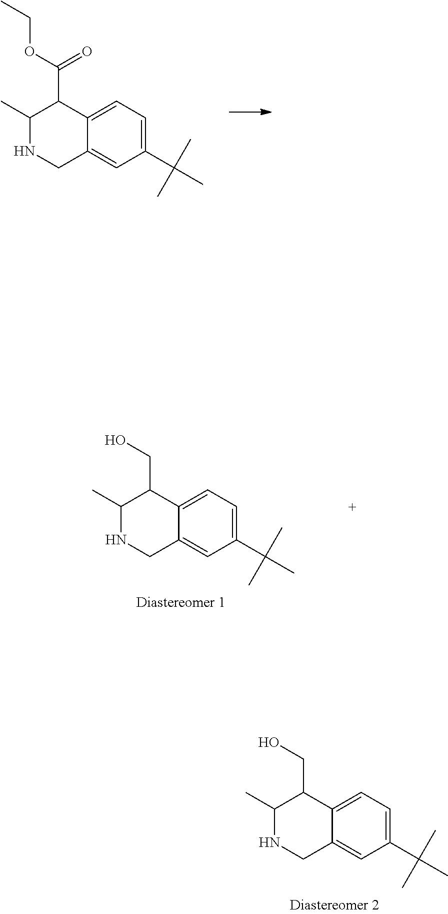 [SMILES: CCOC(=O)C1c2ccc(C(C)(C)C)cc2CNC1C ; CC1NCc2cc(C(C)(C)C)ccc2C1CO ; CC1NCc2cc(C(C)(C)C)ccc2C1CO]

To a suspension of LAH (1.48 g, 38.994 mmol) in THF (50 mL) at 0° C. was added a solution of ethyl 7-tert-butyl-3-methyl-1,2,3,4-tetrahydroisoquinoline-4-carboxylate (2.68 g, 9.7318 mmol) in THE (50 mL). The mixture was stirred at rt for 2 h. The mixture was cooled to 0° C., diluted with THE (100 mL), treated with a solution of 1.5 mL of water in THE (20 mL), 1.5 g of 25% aq. NaOH and 4.5 g of water respectively. The mixture was stirred at rt for 30 min, filtered through diatomaceous earth and washed with THF. The filtrate was dried with sodium sulfate and purified by flash chromatography (80 g silica gel, DCM (1% NH₃)/MeOH 0-10%) to afford the less polar product, diastereomer 1 (7-tert-butyl-3-methyl-1,2,3,4-tetrahydroisoquinolin-4-yl)methanol (1.98 g, 87%) as light yellow oil. ESI-MS m/z calc. 233.178, found 234.2 (M+1)⁺; Retention time: 1.87 minutes (LC method U). ¹H NMR (300 MHz, CDCl₃) δ 7.29-7.23 (m, 1H), 7.18-7.12 (m, 1H), 7.07 (d, J=1.5 Hz, 1H), 4.35 (dd, J=10.4, 2.5 Hz, 1H), 4.22-4.03 (m, 2H), 3.85-3.74 (m, 1H), 3.32-3.19 (m, 1H), 2.54 (d, J=2.3 Hz, 1H), 1.41 (d, J=6.5 Hz, 3H), 1.30 (s, 9H); and as the more polar product, diastereomer 2 (7-tert-butyl-3-methyl-1,2,3,4-tetrahydroisoquinolin-4-yl)methanol (0.29 g, 13%) as light yellow oil. ESI-MS m/z calc. 233.178, found 234.2 (M+1)⁺; Retention time: 1.87 minutes (LC method U). ¹H NMR (300 MHz, CDCl₃) δ 7.26-7.20 (m, 1H), 7.19-7.11 (m, 1H), 7.04 (d, J=1.5 Hz, 1H), 4.13-4.00 (m, 2H), 3.98-3.86 (m, 2H), 3.71-3.64 (m, 1H), 2.56 (q, J=2.9 Hz, 1H), 1.68 (dt, J=6.0, 2.9 Hz, 1H), 1.29 (s, 9H), 1.19 (d, J=6.5 Hz, 3H). TLC (DCM-MeOH 10:1, two drops of 28% aq. NH₃): The major product (Rf=0.5) is much less polar than the minor product (R_{f}=0.25).

### Step 5: (7-tert-Butyl-3-methyl-1,2,3,4-tetrahydroisoquinolin-4-yl)methanol, diastereomer 2, salt formation

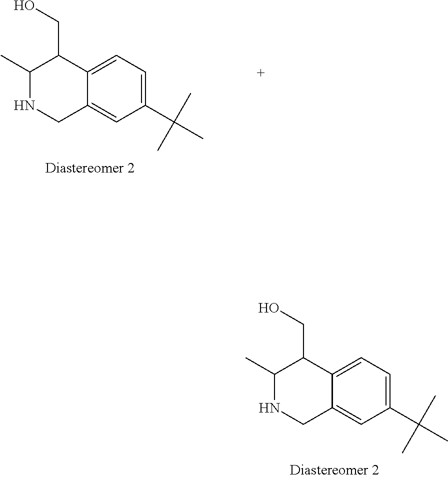 [SMILES: CC1NCc2cc(C(C)(C)C)ccc2C1CO ; CC1NCc2cc(C(C)(C)C)ccc2C1CO]

To a solution of (7-tert-butyl-3-methyl-1,2,3,4-tetrahydroisoquinolin-4-yl)methanol, diastereomer 2 (0.3 g, 1.2856 mmol) in DCM (6 mL) at 0° C. was added HCl (0.4 mL of 4 M, 1.6000 mmol) in 1,4-dioxane. The mixture was stirred at rt for 10 min. Heptanes (50 mL) was added slowly. The mixture was concentrated and freeze dried to give (7-tert-butyl-3-methyl-1,2,3,4-tetrahydroisoquinolin-4-yl)methanol (hydrochloride salt), diastereomer 2 (326 mg, 87%) as a sticky oil. ¹H NMR (300 MHz, DMSO-d₆) δ 9.49 (br. s., 1H), 9.38 (br. s., 1H), 7.51-7.02 (m, 3H), 6.74 (br. s., 1H), 4.32-4.00 (m, 2H), 3.83-3.60 (m, 2H), 3.48-3.32 (m, 1H), 2.82 (q, J=5.0 Hz, 1H), 1.49-1.34 (m, 3H), 1.26 (s, 9H). ESI-MS m/z calc. 233.17796, found 234.2 (M+1)⁺; Retention time: 1.9 minutes; LC method U.

### Step 6: 21-tert-Butyl-12-(2,6-dimethylphenyl)-25-methyl-15-oxa-8λ⁶-thia-1,9,11,26-tetraazapentacyclo[15.7.1.13,7.110,14.018,23]heptacosa-3,5,7(27),10,12,14(26),18,20,22-nonaene-2,8,8-trione, enantiomer 1 (Compound 218), and 21-tert-butyl-12-(2,6-dimethylphenyl)-25-methyl-15-oxa-8λ⁶-thia-1,9,11,26-tetraazapentacyclo[15.7.1.13,7.110,14.018,23]heptacosa-3,5,7(27),10,12,14(26),18,20,22-nonaene-2,8,8-trione, enantiomer 2 (Compound 219)

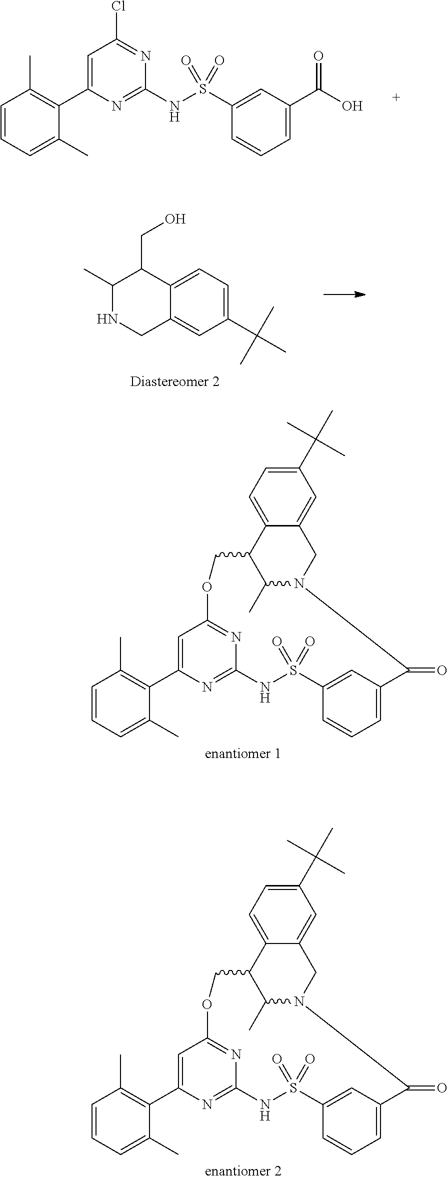 [SMILES: Cc1cccc(C)c1-c1cc(Cl)nc(NS(=O)(=O)c2cccc(C(=O)O)c2)n1 ; CC1NCc2cc(C(C)(C)C)ccc2C1CO ; Cc1cccc(C)c1-c1cc2nc(n1)NS(=O)(=O)c1cccc(c1)C(=O)N1Cc3cc(C(C)(C)C)ccc3C(CO2)C1C ; Cc1cccc(C)c1-c1cc2nc(n1)NS(=O)(=O)c1cccc(c1)C(=O)N1Cc3cc(C(C)(C)C)ccc3C(CO2)C1C]

To a solution of 3-[[4-chloro-6-(2,6-dimethylphenyl)pyrimidin-2-yl]sulfamoyl]benzoic acid (approximately 633.9 mg, 1.517 mmol) and (7-tert-butyl-3-methyl-1,2,3,4-tetrahydroisoquinolin-4-yl)methanol (hydrochloride salt) diastereomer 2 (315 mg, 1.167 mmol) (minor diastereomeric product from previous reaction) in THE (11.67 mL) was added potassium tert-butoxide (approximately 654.8 mg, 725.9 μL, 5.835 mmol). The reaction was stirred for 2 hours and quenched with the addition of hydrochloric acid (approximately 534.9 μL of 12 M, 6.419 mmol). The sample was purified by reverse phase HPLC (Waters Sunfire Cis column (100×50 mm, 10 m particle size), gradient: 1-99% acetonitrile in water (5 mM HCl) over 15.0 minutes) which afforded 3-[[4-[(7-tert-butyl-3-methyl-1,2,3,4-tetrahydroisoquinolin-4-yl)methoxy]-6-(2,6-dimethylphenyl)pyrimidin-2-yl]sulfamoyl]benzoic acid (hydrochloride salt) (175 mg, 23%) ESI-MS m/z calc. 614.2563, Retention time: 0.56 minutes (LC method D). To a solution of the product in DMF (6 mL) was added HATU (approximately 576.8 mg, 1.517 mmol). The reaction was heated to 50° C. for 10 min. triethylamine (approximately 354.3 mg, 488.0 μL, 3.501 mmol) was added and the reaction was further stirred at this temperature for 20 min. The sample was purified by reverse phase HPLC (Waters Sunfire C₁₈ column (100×50 mm, 10 m particle size), gradient: 1-99% acetonitrile in water (5 mM HCl) over 15.0 minutes). The lyophilized fractions were further separated by semi-preparative chiral SFC (1 to 99% methanol in carbon dioxide, column: Daicel Chiralpak RR, 250×20 mm, 5 μM) which afforded enantiomer 1,21-tert-butyl-12-(2,6-dimethylphenyl)-25-methyl-15-oxa-8λ⁶-thia-1,9,11,26-tetraazapentacyclo[15.7.1.13,7.110,14.018,23]heptacosa-3,5,7(27),10,12,14(26),18,20,22-nonaene-2,8,8-trione (5.7 mg, 2%) ESI-MS m/z calc. 596.2457, found 597.51 (M+1)⁺; Retention time: 0.82 minutes (LC method D); and enantiomer 2, 21-tert-butyl-12-(2,6-dimethylphenyl)-25-methyl-15-oxa-8λ⁶-thia-1,9,11,26-tetraazapentacyclo[15.7.1.13,7.110,14.018,23]heptacosa-3,5,7(27),10,12,14(26),18,20,22-nonaene-2,8,8-trione (4.8 mg, 1%) ESI-MS m/z calc. 596.2457, found 597.51 (M+1)⁺; Retention time: 0.82 minutes (LC method D).

### Example 119: Preparation of Compound 220

### Step 1: Methyl 2-(trifluoromethylsulfonylamino)acetate

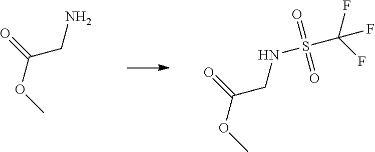

Triethylamine (22.6 mL, 163.2 mmol) was added to a solution of methyl 2-aminoacetate hydrochloride (10.0 g, 79.6 mmol) in dichloromethane (468 mL) at room temperature and the solution was stirred for 15 minutes at room temperature and then cooled to −78 C. Triflic anhydride (14 mL, 83.6 mmol) was added slowly and the solution was allowed to slowly warm to room temperature. The reaction was stirred for three days. The organic layer was separated and washed with 4 M hydrochloric acid (2×200 mL), aqueous saturated sodium chloride solution (200 mL), dried over sodium sulfate and concentrated to afford methyl 2-(trifluoromethylsulfonamido)acetate (10.6 g, 47.8 mmol) as a white powder. ¹H NMR (250 MHz, CDCl₃) δ 5.67 (bs, 1H), 4.07 (s, 2H), 3.83 (s, 3H).

### Step 2: Methyl 2-[2-phenylethyl(trifluoromethylsulfonyl)amino]acetate

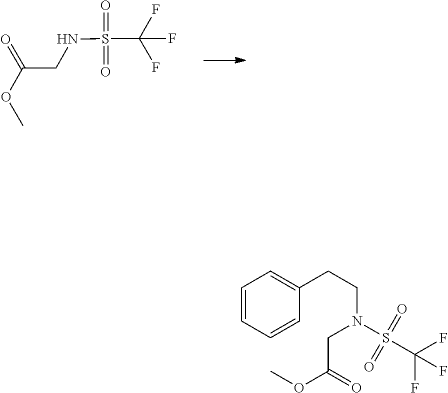 [SMILES: COC(=O)CNS(=O)(=O)C(F)(F)F ; COC(=O)CN(CCc1ccccc1)S(=O)(=O)C(F)(F)F]

To a solution of methyl 2-(trifluoromethylsulfonamido)acetate (1.0 g, 4.52 mmol) and 2-phenylethanol (0.54 mL, 4.52 mmol) in tetrahydrofuran (18.8 mL) at 0 C was added triphenylphosphine (1.42 g, 5.42 mmol) and allowed to stir for 15 minutes. At 0° C., diisopropyl azodicarboxylate (1.07 mL, 5.42 mmol) was added dropwise and the reaction mixture was stirred for 18 hours at room temperature. The volatiles were removed under vacuum and the crude residue was purified by silica gel column chromatography using 0-70% hexanes-ethyl acetate to give methyl 2-(1,1,1-trifluoro-N-phenethylmethylsulfonamido)acetate (1.06 g, 81%) as a yellow oil. ¹H NMR (250 MHz, CDCl₃) δ 6.96-7.63 (m, 5H) 4.01 (br. s., 2H) 3.67-3.87 (m, 5H) 2.95 (t, J=7.75 Hz, 2H). ESI-MS m/z calc. 325.05957, found 326.3 (M+1)⁺; Retention time: 3.56 minutes.

### Step 3: 2-[2-Phenylethyl(trifluoromethylsulfonyl)amino]acetic acid

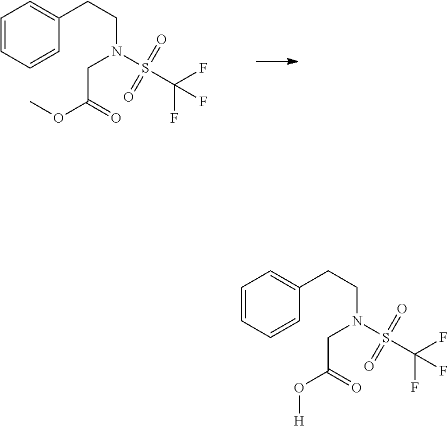 [SMILES: COC(=O)CN(CCc1ccccc1)S(=O)(=O)C(F)(F)F ; O=C(O)CN(CCc1ccccc1)S(=O)(=O)C(F)(F)F]

A solution of methyl 2-(1,1,1-trifluoro-N-phenethylmethylsulfonamido)acetate (assumed 76 mmol) in a mixture of tetrahydrofuran (266 mL) and water (114 mL) was prepared. Lithium hydroxide (14.5 g, 604 mmol) was added and the reaction was allowed to stir for 16 hours at room temperature. The reaction was quenched with 2 M aqueous hydrochloric acid and the layers separated. The aqueous layer was extracted three times with dichloromethane (3×100 mL). The combined organic layer was dried over sodium sulfate and concentrated to give 2-(1,1,1-trifluoro-N-phenethylmethylsulfonamido)acetic acid (12 g, 51%) as a white solid. ¹H NMR (250 MHz, CDCl₃) δ 7.02-7.46 (m, 5H) 5.08 (br. s., 1H) 4.06 (br. s., 2H) 3.73 (br. s., 2H) 2.96 (t, J=7.8 Hz, 2H). ESI-MS m/z calc. 311.0439, found 312.2 (M+1)⁺; Retention time: 3.4 minutes.

### Step 4: 3-(Trifluoromethylsulfonyl)-2,4-dihydro-1H-3-benzazepin-5-one

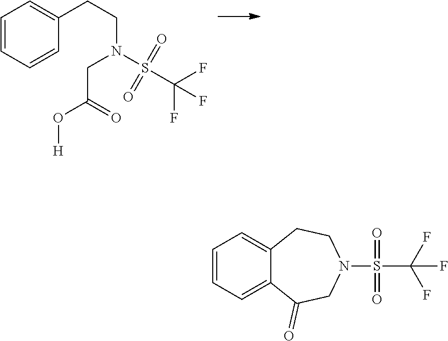 [SMILES: O=C(O)CN(CCc1ccccc1)S(=O)(=O)C(F)(F)F ; O=C1CN(S(=O)(=O)C(F)(F)F)CCc2ccccc21]

A solution of 2-(1,1,1-trifluoro-N-phenethylmethylsulfonamido)acetic acid (12 g, 38.6 mmol) in 1,2-dichloroethane (386 mL) was cooled to 0° C. in an ice bath. Phosphorous pentoxide (54.8 g, 193 mmol) was added and the reaction mixture was allowed to warm to room temperature and stirred for 18 hours. The reaction as quenched by the addition of 2 M aqueous sodium hydroxide. The layers were separated, and the aqueous layer was extracted with dichloromethane (3×80 mL). The combined organic layers were dried over sodium sulfate and concentrated. The crude residue was purified by silica gel column chromatography using 0-30% hexanes-ethyl acetate to give 3-((trifluoromethyl)sulfonyl)-2,3,4,5-tetrahydro-1H-benzo[d]azepin-1-one (6 g, 50%) as a white solid. ¹H NMR (250 MHz, CDCl₃) δ 7.79 (d, J=8.2 Hz, 1H) 7.58 (m, J 7.5 Hz, 1H) 7.40-7.51 (m, 1H) 7.23-7.31 (m, 1H) 4.34 (s, 2H) 3.84 (br. s., 2H) 3.13 (t, J=6.7 Hz, 2H). ESI-MS m/z calc. 293.03336, found 294.4 (M+1)⁺; Retention time: 2.92 minutes; LC method B.

### Step 5: 2,3,4,5-Tetrahydro-1H-3-benzazepin-5-ol

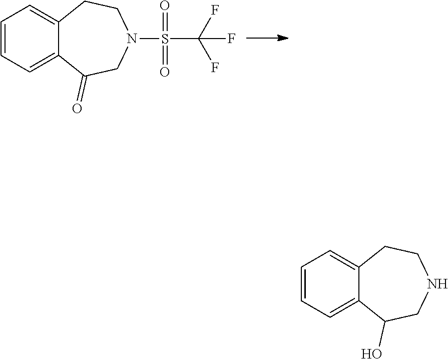 [SMILES: O=C1CN(S(=O)(=O)C(F)(F)F)CCc2ccccc21 ; OC1CNCCc2ccccc21]

A solution of 3-((trifluoromethyl)sulfonyl)-2,3,4,5-tetrahydro-1H-benzo[d]azepin-1-one (5.8 g, 30.05 mmol) in a mixture of toluene (100 mL) and tetrahydrofuran (50 mL) was cooled to 0° C. and lithium aluminum hydride powder (5.7 g, 150.24 mmol) was added slowly. The reaction mixture was stirred at 70° C. for 17 hours. The reaction was cooled to 0° C. and an aqueous saturated sodium sulfate solution (150 mL) was added over 1 hour. The formed solids were filtered off and washed with some chloroform and methanol. The filtrate was concentrated to give 2,3,4,5-tetrahydro-1H-benzo[d]azepin-1-ol (2.74 g, 85%) as a white solid. ¹H NMR (250 MHz, CDCl₃) δ 7.02-7.36 (m, 4H) 4.62 (d, J=6.3 Hz, 1H) 3.14-3.40 (m, 3H) 2.62-2.98 (m, 3H). ESI-MS m/z calc. 163.09972, found 164.6 (M+1)⁺; Retention time: 1.19 minutes; LC method B.

### Step 6: tert-Butyl 5-hydroxy-1,2,4,5-tetrahydro-3-benzazepine-3-carboxylate

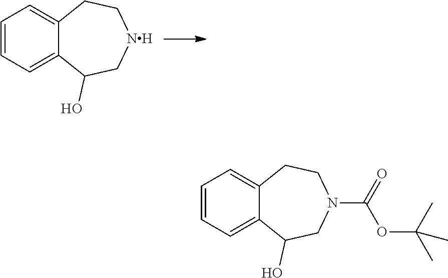

To a mixture of 2,3,4,5-tetrahydro-1H-benzo[d]azepin-1-ol (2.25 g, 13.8 mmol) in a mixture of tetrahydrofuran (34.5 mL) and 1 M aqueous sodium hydroxide (34.5 mL) was added di-tert-butyl dicarbonate (3.32 g, 15.2 mmol) and the reaction mixture was stirred for 2 hours at room temperature. The reaction was diluted with water (200 mL) and ethyl acetate (200 mL). The layers were separated, and the aqueous layer was extracted with ethyl acetate (3×50 mL). The combined organic layer was dried over sodium sulfate and concentrated. The crude residue was purified by silica gel column chromatography using 0-35% hexanes-ethyl acetate to give tert-butyl 1-hydroxy-4,5-dihydro-1H-benzo[d]azepine-3(2H)-carboxylate (3.14 g, 71%) as a white solid. ¹H NMR (250 MHz, CDCl₃) δ 7.30-7.41 (m, 1H) 7.16-7.26 (m, 2H) 7.07-7.16 (m, 1H) 4.85 (br. s., 1H) 3.95-4.20 (m, 1H) 3.85 (dd, J=11.8, 7.0 Hz, 2H), 3.39-3.64 (m, 1H), 3.11-3.63 (m, 2H) 2.68-2.86 (m, 1H) 1.47 (s, 9H). ESI-MS m/z calc. 263.15213, found 264.4 (M+1)⁺; Retention time: 2.71 minutes; LC method B.

### Step 7: 12-(2,6-Dimethylphenyl)-15-oxa-8λ⁶-thia-1,9,11,26-tetraazapentacyclo[14.8.1.13,7.110,14.017,22]heptacosa-3(27),4,6,10(26),11,13,17,19,21-nonaene-2,8,8-trione (Compound 220)

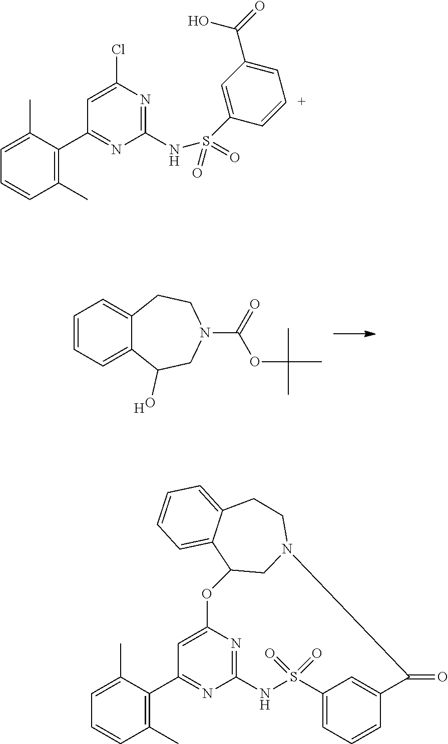

3-[[4-Chloro-6-(2,6-dimethylphenyl)pyrimidin-2-yl]sulfamoyl]benzoic acid (125 mg, 0.2991 mmol), tert-butyl 5-hydroxy-1,2,4,5-tetrahydro-3-benzazepine-3-carboxylate (103.4 mg), and NaH (70 mg, 1.750 mmol) were combined in anhydrous NMP (2 mL), and stirred for 1 hour at room temperature. The reaction mixture was then quenched with 0.2 mL acetic acid, diluted with methanol, filtered, and purified by reverse phase HPLC (1-70% ACN HCl modifier, 15 min run), to give 3-[[4-[(3-tert-butoxycarbonyl-1,2,4,5-tetrahydro-3-benzazepin-5-yl)oxy]-6-(2,6-dimethylphenyl)pyrimidin-2-yl]sulfamoyl]benzoic acid (113 mg, 59%) ESI-MS m/z calc. 644.23047, found 645.4 (M+1)⁺; Retention time: 0.71 minutes (LC method D). The product was dissolved in dichloromethane (2 mL), and HCl (2 mL of 4 M, 8.000 mmol) in dioxane was added. After stirring at room temperature for 1 hour, the reaction mixture was concentrated under vacuum to give 3-[[4-(2,6-dimethylphenyl)-6-(2,3,4,5-tetrahydro-1H-3-benzazepin-5-yloxy)pyrimidin-2-yl]sulfamoyl]benzoic acid (hydrochloride salt) (104 mg, 60%). This product was combined with HATU (90 mg, 0.2367 mmol) in DMF (12 mL), and DIPEA (156 μL, 0.8956 mmol) was added. After stirring for one hour at room temperature, the reaction mixture was diluted with ethyl acetate and 0.5 M HCl, and the layers were separated. The aqueous layer was extracted an additional time with ethyl acetate, then the combined ethyl acetate layers were washed with water, brine and dried over sodium sulfate then concentrated. This crude material was then purified by chromatography on silica gel 0-10% methanol, dichloromethane to give a white solid 12-(2,6-dimethylphenyl)-15-oxa-8λ⁶-thia-1,9,11,26-tetraazapentacyclo[14.8.1.13,7.110,14.017,22]heptacosa-3(27),4,6,10(26),11,13,17,19,21-nonaene-2,8,8-trione (75 mg, 48%) ESI-MS m/z calc. 526.1675, found 527.3 (M+1)⁺; Retention time: 1.63 minutes. 10 mg of this material was further purified by reverse phase HPLC (1-70% ACN in water, HCl modifier 15 min run) to give 12-(2,6-dimethylphenyl)-15-oxa-8λ⁶-thia-1,9,11,26-tetraazapentacyclo[14.8.1.13,7.110,14.017,22]heptacosa-3(27),4,6,10(26),11,13,17,19,21-nonaene-2,8,8-trione (8.6 mg, 5%) ESI-MS m/z calc. 526.1675, found 527.4 (M+1)⁺; Retention time: 1.63 minutes (LC method A).

### Example 120: Preparation of Compound 221 and Compound 222

### Step 1: 12-(2,6-dimethylphenyl)-15-oxa-8λ⁶-thia-1,9,11,26-tetraazapentacyclo[14.8.1.13,7.110,14.017,22]heptacosa-3(27),4,6,10(26),11,13,17,19,21-nonaene-2,8,8-trione, enantiomer 1 (Compound 222), and 12-(2,6-dimethylphenyl)-15-oxa-8λ⁶-thia-1,9,11,26-tetraazapentacyclo[14.8.1.13,7.110,14.017,22]heptacosa-3(27),4,6,10(26),11,13,17,19,21-nonaene-2,8,8-trione, enantiomer 2 (Compound 221)

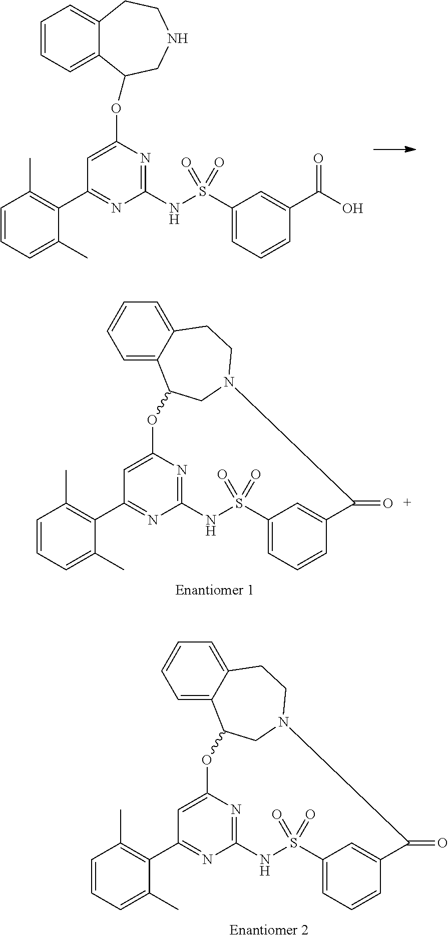

A solution of 3-[[4-(2,6-dimethylphenyl)-6-(2,3,4,5-tetrahydro-1H-3-benzazepin-5-yloxy)pyrimidin-2-yl]sulfamoyl]benzoic acid (hydrochloride salt) (105 mg, 0.1807 mmol) and HATU (103 mg, 0.2709 mmol) in DMF (4.5 mL) was cooled in an ice bath. DIPEA (100 μL, 0.5741 mmol) was added and the mixture was stirred at RT for 1 h, filtered and purified by preparative reverse phase HPLC (Cis): 1-99% ACN in water/HCl modifier (15 min) to afford 12-(2,6-dimethylphenyl)-15-oxa-8λ⁶-thia-1,9,11,26-tetraazapentacyclo[14.8.1.13,7.110,14.017,22]heptacosa-3(27),4,6,10(26),11,13,17,19,21-nonaene-2,8,8-trione (56.2 mg, 59%) ESI-MS m/z calc. 526.1675, found 527.3 (M+1)⁺; Retention time: 1.64 minutes (LC method A). The mixture was subjected to SFC using a chiral AD column to give enantiomer 1, peak 1, 12-(2,6-dimethylphenyl)-15-oxa-8λ⁶-thia-1,9,11,26-tetraazapentacyclo[14.8.1.13,7.110,14.017,22]heptacosa-3(27),4,6,10(26),11,13,17,19,21-nonaene-2,8,8-trione (5.9 mg, 6%) ESI-MS m/z calc. 526.1675, found 527.3 (M+1)⁺; Retention time: 1.64 minutes (LC method A); and enantiomer 2, peak 2, 12-(2,6-dimethylphenyl)-15-oxa-8λ⁶-thia-1,9,11,26-tetraazapentacyclo[14.8.1.13,7.110,14.017,22]heptacosa-3(27),4,6,10(26),11,13,17,19,21-nonaene-2,8,8-trione (7.1 mg, 7%) ESI-MS m/z calc. 526.1675, found 527.41 (M+1)⁺; Retention time: 1.64 minutes (LC method A).

### Example 121: Preparation of Compound 223 and Compound 224

### Step 1: tert-Butyl 3-oxo-5-phenyl-piperidine-1-carboxylate

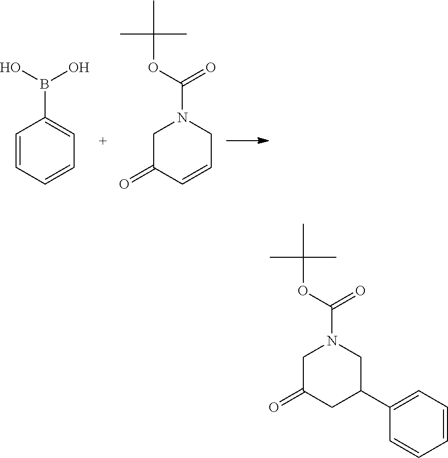

A solution of tert-butyl 3-oxo-2,6-dihydropyridine-1-carboxylate (1.58 g, 8.0109 mmol), phenylboronic acid (1.96 g, 16.075 mmol) and 2-(4,4-dimethyl-4,5-dihydro-2-oxazolyl)pyridine (174 mg, 0.9874 mmol) in dichloroethane (35 mL) was purged with nitrogen for 5 minutes. Palladium(II) trifluoroacetate (269 mg, 0.8091 mmol) was added, the tube was sealed and reaction mixture was heated at 60° C. overnight. Once cooled to room temperature, the reaction mixture was concentrated under reduced pressure. The residue was purified by silica gel chromatography using 0% to 50% EtOAc in heptanes to afford tert-butyl 3-oxo-5-phenyl-piperidine-1-carboxylate (1.35 g, 61%) as a white solid. ¹H NMR (300 MHz, CDCl₃) δ 1.43 (br. s., 9H), 2.56-2.91 (m, 2H), 3.17-3.61 (m, 2H), 3.79-4.42 (m, 3H), 7.04-7.49 (m, 5H). ESI-MS m/z calc. 275.1521, found 220.1 (M-C₄H₈+1)⁺; Retention time: 2.2 minutes (LC method O).

### Step 2: tert-Butyl 3-hydroxy-5-phenyl-piperidine-1-carboxylate

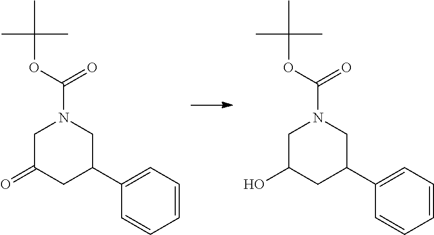 [SMILES: CC(C)(C)OC(=O)N1CC(=O)CC(c2ccccc2)C1 ; CC(C)(C)OC(=O)N1CC(O)CC(c2ccccc2)C1]

Sodium borohydride (270 mg, 7.1367 mmol) was added to a solution of tert-butyl 3-oxo-5-phenyl-piperidine-1-carboxylate (1.94 g, 7.0458 mmol) in methanol (20 mL) at 0° C. and reaction mixture was stirred at same temperature for 1 hour. Reaction mixture was quenched with aqueous saturated ammonium chloride (100 mL) and aqueous layer was extracted using ethyl acetate (3×100 mL). Organic layers were combined, dried over sodium sulfate and concentrated under reduced pressure. The residue was purified by silica gel chromatography using 0% to 30% of EtOAc in heptanes to afford tert-butyl 3-hydroxy-5-phenyl-piperidine-1-carboxylate (1.4 g, 69%) as white solid. ¹H NMR (300 MHz, CDCl₃) δ 1.47 (s, 9H), 1.55-1.72 (m, 2H), 2.23-2.37 (m, 1H), 2.45-2.86 (m, 3H), 3.70-3.88 (m, 1H), 4.04-4.46 (m, 2H), 7.18-7.41 (m, 5H). ESI-MS m/z calc. 277.1678, found 300.2 (M+Na)⁺; Retention time: 4.18 minutes (LC method G).

### Step 3: tert-Butyl 3-(4-nitrobenzoyl)oxy-5-phenyl-piperidine-1-carboxylate

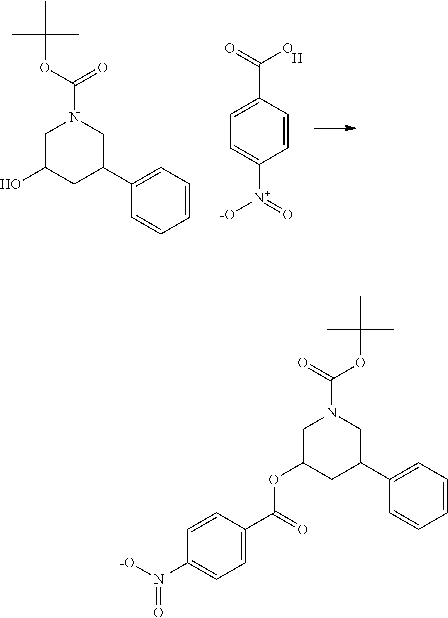

A solution of diisopropyl azodicarboxylate (92.430 mg, 0.09 mL, 0.4571 mmol) in tetrahydrofuran (1 mL) was slowly added to a solution of tert-butyl 3-hydroxy-5-phenyl-piperidine-1-carboxylate (100 mg, 0.3605 mmol), triphenylphosphine (114 mg, 0.1007 mL, 0.4346 mmol) and 4-nitrobenzoic acid (73 mg, 0.4368 mmol) in tetrahydrofuran (5 mL) and reaction mixture was stirred at room temperature overnight. The reaction mixture was diluted with water (2 mL) and concentrated under reduced pressure. The residue was diluted with water (10 mL) and extracted using diethyl ether (3×10 mL). Organic layers were combined, washed with water (10 mL) and brine (10 mL), dried over sodium sulfate and concentrated under reduced pressure. The residue was purified by silica gel chromatography using 0% to 15% of EtOAc in heptanes to afford tert-butyl 3-(4-nitrobenzoyl)oxy-5-phenyl-piperidine-1-carboxylate (106 mg, 69%) as a white solid. ¹H NMR (300 MHz, CDCl₃) δ 1.12-1.34 (m, 10H), 1.94-2.14 (m, 1H), 2.38 (d, J=14.1 Hz, 1H), 2.99-3.36 (m, 2H), 4.19-4.69 (m, 2H), 5.22-5.42 (m, 1H), 7.20-7.42 (m, 5H), 8.15-8.39 (m, 4H). ESI-MS m/z calc. 426.1791, found 371.1 (M-C₄H₈+1)⁺+; Retention time: 2.51 minutes (LC method C).

### Step 4: tert-Butyl 3-hydroxy-5-phenyl-piperidine-1-carboxylate

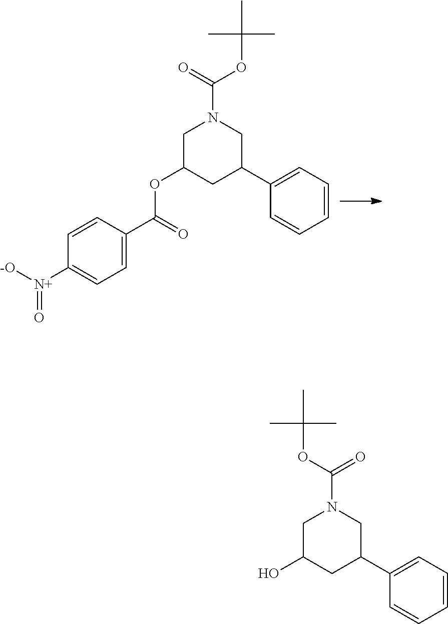

Lithium hydroxide (0.5 mL of 1 M, 0.5000 mmol) was added to a solution of tert-butyl 3-(4-nitrobenzoyl)oxy-5-phenyl-piperidine-1-carboxylate (106 mg, 0.2486 mmol) in tetrahydrofuran (3 mL) and the reaction mixture was stirred at room temperature overnight. The reaction mixture was diluted with water (20 mL) and extracted using ethyl acetate (3×10 mL). Organic layers were combined, dried over sodium sulfate and concentrated under reduced pressure to afford tert-butyl 3-hydroxy-5-phenyl-piperidine-1-carboxylate (59 mg, 86%) as a white solid. ¹H NMR (400 MHz, CDCl₃) δ 1.42 (s, 9H), 1.82-1.90 (m, 2H), 2.87-2.99 (m, 1H), 3.05 (dd, J=13.3, 2.1 Hz, 1H), 3.07-3.21 (m, 1H), 3.78-3.84 (m, 1H), 3.87 (dq, J=6.1, 3.1 Hz, 1H), 3.93 (dd, J=13.0, 3.7 Hz, 1H), 4.33-4.45 (m, 1H), 7.16-7.40 (m, 5H). ESI-MS m/z calc. 277.1678, found 222.2 (M-C₄H₈+1)⁺+; Retention time: 1.97 minutes (LC method C).

### Step 5: 18-(2,6-dimethylphenyl)-5-phenyl-2-oxa-14λ⁶-thia-7,15,17,20-tetraazatetracyclo[14.3.1.13,7.19,13]docosa-1(19),9(21),10,12,16(20),17-hexaene-8,14,14-trione, diastereomer 1 (Compound 223), and 18-(2,6-dimethylphenyl)-5-phenyl-2-oxa-14λ⁶-thia-7,15,17,20-tetraazatetracyclo[14.3.1.13,7.19,13]docosa-1(19),9(21),10,12,16(20),17-hexaene-8,14,14-trione, diastereomer 2 (Compound 224)

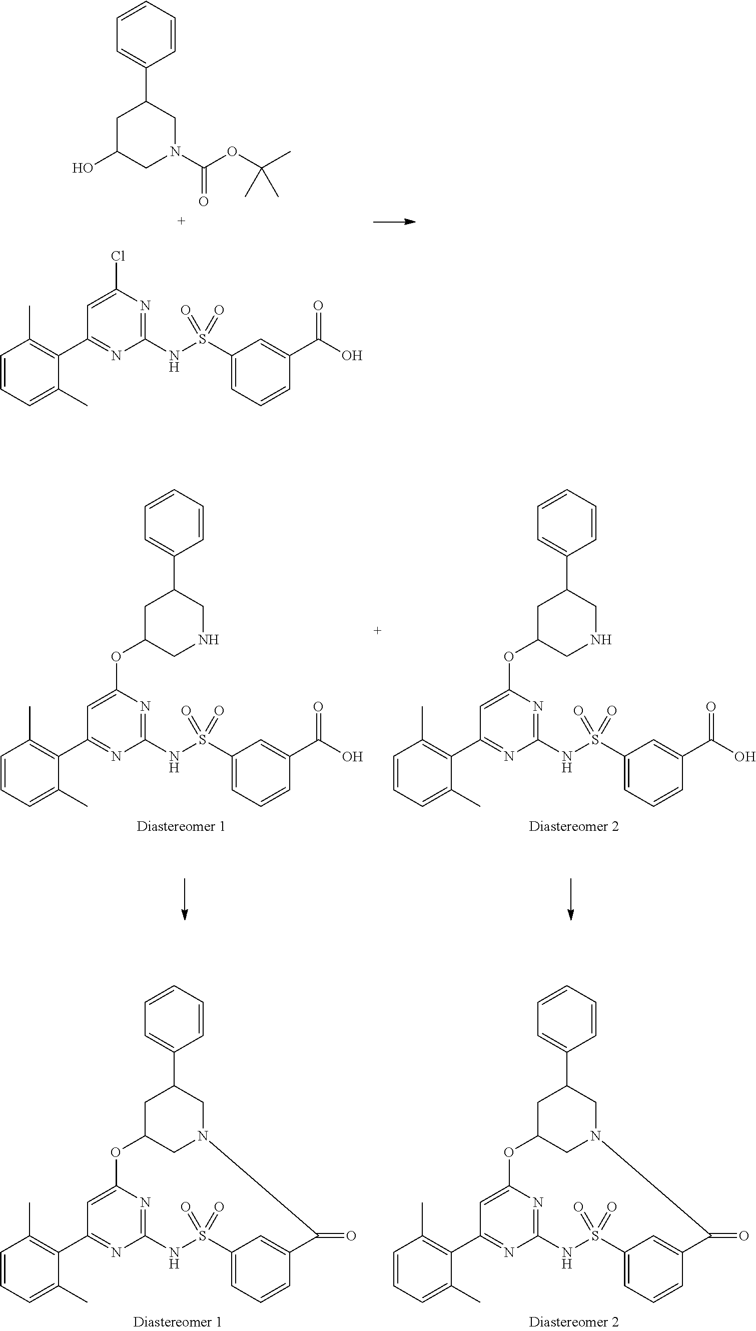

In a 3-mL vial, tert-butyl 3-hydroxy-5-phenyl-piperidine-1-carboxylate (60.1 mg, 0.2080 mmol) and 3-[[4-chloro-6-(2,6-dimethylphenyl)pyrimidin-2-yl]sulfamoyl]benzoic acid (80 mg, 0.1914 mmol) were dissolved in NMP (1.0 mL), to which NaH (35 mg of 60% w/w, 0.8751 mmol) was added. This mixture was stirred at room temperature for 1.5 h, after which it was quenched dropwise with 1N HCl (1.0 mL) and extracted with ethyl acetate (3×2 mL). The combined organic extracts were washed with water (4 mL) and saturated aqueous sodium chloride solution (4 mL), then dried over sodium sulfate, filtered, and evaporated in vacuo. The crude product, 3-[[4-[(1-tert-butoxycarbonyl-5-phenyl-3-piperidyl)oxy]-6-(2,6-dimethylphenyl)pyrimidin-2-yl]sulfamoyl]benzoic acid (ca. 200 mg), was taken onto the next step without further purification. In a 3-mL vial, the crude product from above was dissolved in dioxane (0.75 mL) and treated with a dioxane solution of HCl (0.25 mL of 4.0 M, 1.000 mmol). This mixture was stirred at room temperature for 15 min then at 50° C. for 1.5 h. It was then cooled to room temperature, diluted with 1:1 MeOH:DMSO (1 mL), filtered and purified by reverse phase HPLC (1-50% acetonitrile in water using HCl as a modifier) to give two separable diastereomers: Diastereomer 1, 3-[[4-(2,6-dimethylphenyl)-6-[(5-phenyl-3-piperidyl)oxy]pyrimidin-2-yl]sulfamoyl]benzoic acid (hydrochloride salt) (4.5 mg, 4%) ESI-MS m/z calc. 558.19366, found 559.4 (M+1)⁺; Retention time: 1.14 minutes (LC method A); and diastereomer 2, 3-[[4-(2,6-dimethylphenyl)-6-[(5-phenyl-3-piperidyl)oxy]pyrimidin-2-yl]sulfamoyl]benzoic acid (hydrochloride salt) (73.7 mg, 65%) ESI-MS m/z calc. 558.19366, found 559.4 (M+1)⁺; Retention time: 1.25 minutes (LC method A). Each diastereomer was macrocyclized separately.

In a 3-mL vial, diastereomer 1 was dissolved in DMF (200 μL), and treated with DIPEA (25 μL, 0.14 mmol) and HATU (7.5 mg, 0.020 mmol). This mixture was stirred at room temperature for 5 min, after which it was diluted with MeOH (200 μL), filtered and purified by reverse phase HPLC (1-70% acetonitrile in water using HCl as a modifier) to give “diastereomer 1”, 18-(2,6-dimethylphenyl)-5-phenyl-2-oxa-14λ⁶-thia-7,15,17,20-tetraazatetracyclo[14.3.1.13,7.19,13]docosa-1(19),9(21),10,12,16(20),17-hexaene-8,14,14-trione (0.9 mg, 1%) ESI-MS m/z calc. 540.1831, found 541.4 (M+1)⁺; Retention time: 1.64 minutes (LC method A).

In a 3-mL vial, diastereomer 2 from was dissolved in DMF (2.0 mL), and treated with DIPEA (0.25 mL, 1.435 mmol) and HATU (75 mg, 0.1972 mmol). This mixture was stirred at room temperature for 5 min, after which it was diluted with MeOH (0.3 mL), filtered and purified by reverse phase HPLC (1-70% acetonitrile in water using HCl as a modifier) to give “diastereomer 2”, 18-(2,6-dimethylphenyl)-5-phenyl-2-oxa-14λ⁶-thia-7,15,17,20-tetraazatetracyclo[14.3.1.13,7.19,13]docosa-1(19),9(21),10,12,16(20),17-hexaene-8,14,14-trione (32.5 mg, 31%) ¹H NMR (400 MHz, dimethylsulfoxide-d₆) δ 13.46-11.55 (bs, 1H), 8.59 (s, 1H), 8.03-7.88 (m, 1H), 7.81-7.62 (m, 2H), 7.48-7.42 (m, 2H), 7.41-7.36 (m, 2H), 7.33-7.28 (m, 1H), 7.26 (t, J=7.7 Hz, 1H), 7.13 (d, J=7.7 Hz, 2H), 6.38 (s, 1H), 5.45-5.32 (m, 1H), 4.51 (dd, J=12.6, 4.3 Hz, 1H), 3.95 (d, J=12.6 Hz, 1H), 3.33-3.24 (m, 1H), 3.03-2.87 (m, 2H), 2.47-2.38 (m, 1H), 2.22 (q, J=11.9 Hz, 1H), 2.15-1.99 (bs, 6H) ESI-MS m/z calc. 540.1831, found 541.1 (M+1)⁺; Retention time: 1.76 minutes (LC method A).

### Example 122: Preparation of Compound 225

### Step 1: (E)-N-Allyl-1-phenyl-methanimine

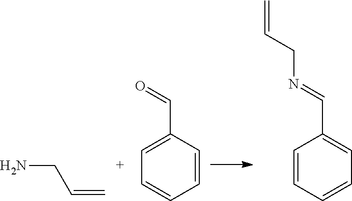

To a solution of benzaldehyde (10 g, 94.231 mmol) in dichloromethane (140 mL) was added prop-2-en-1-amine (6.4685 g, 8.5 mL, 113.29 mmol) and anhydrous magnesium sulfate (9.4 g, 78.094 mmol). The resulting suspension was stirred at room temperature overnight. The reaction mixture was filtered on Celite, washed several times with methyl tert-butyl ether. The filtrate was concentrated under reduced pressure to give crude (E)-N-allyl-1-phenyl-methanimine (12.93 g, 95%) as yellow oil. ¹H NMR (300 MHz, CDCl₃) δ 4.28 (dq, J=5.8, 1.5 Hz, 2H), 5.08-5.34 (m, 2H), 6.09 (ddt, J=17.2, 10.2, 5.7 Hz, 1H), 7.36-7.50 (m, 3H), 7.70-7.83 (m, 2H), 8.24-8.37 (m, 1H).

### Step 2: N-Allyl-1-phenyl-but-3-en-1-amine

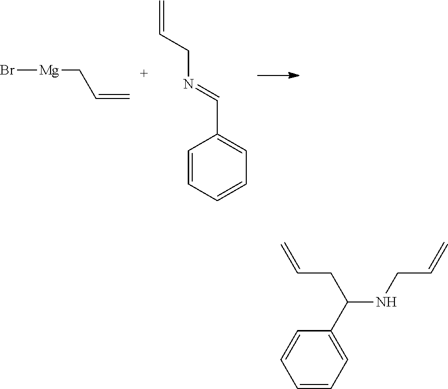

To a solution of (E)-N-allyl-1-phenyl-methanimine (12.93 g, 89.049 mmol) in dichloromethane (140 mL) was added dropwise a solution of allyl(bromo)magnesium in diethyl ether (178 mL of 1 M, 178.00 mmol) and the reaction mixture was stirred at room temperature for 2 days. Saturated aqueous ammonium chloride (50 mL) was slowly added, stirred vigorously and water (150 mL) was added to dissolve the solid. The organic layer was decanted, and the aqueous layer was extracted with methyl tert-butyl ether (2×300 mL). The combined organic layers were washed with brine, dried over sodium sulfate and concentrated under reduced pressure to give crude N-allyl-1-phenyl-but-3-en-1-amine (18.15 g, 109%) as a brown oil. ESI-MS m/z calc. 187.1361, found 188.2 (M+1)⁺; Retention time: 1.3 minutes; LC method P.

### Step 3: tert-Butyl N-allyl-N-(1-phenylbut-3-enyl)carbamate

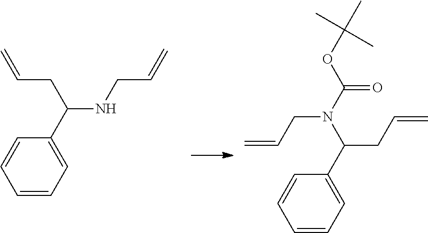 [SMILES: C=CCNC(CC=C)c1ccccc1 ; C=CCC(c1ccccc1)N(CC=C)C(=O)OC(C)(C)C]

To a solution of N-allyl-1-phenyl-but-3-en-1-amine (16.677 g, 89.049 mmol) in dichloromethane (500 mL) was added triethylamine (11.979 g, 16.5 mL, 118.38 mmol) followed by Boc anhydride (31.54 g, 33.200 mL, 144.52 mmol). The resulting mixture was stirred at room temperature overnight. Saturated aqueous ammonium chloride (150 mL) was added and the phases were decanted. The aqueous layer was extracted with methyl tert-butyl ether (150 mL). The combined organic layers were dried over sodium sulfate and concentrated under reduced pressure. The crude was pre-purified on a pad of silica gel eluting with 100% heptanes, then 5% ethyl acetate. One of the fractions was purified on silica gel chromatography, eluting from 0% to 5% ethyl acetate in heptanes to give impure tert-butyl N-allyl-N-(1-phenylbut-3-enyl)carbamate (6.167 g, 24%) as light yellow oil. ESI-MS m/z calc. 287.1885, found 232.2 (M-C₄H₈+1)⁺; Retention time: 2.35 minutes (LC method P).

### Step 4: tert-Butyl 2-phenyl-3,4-dihydro-2H-pyridine-1-carboxylate

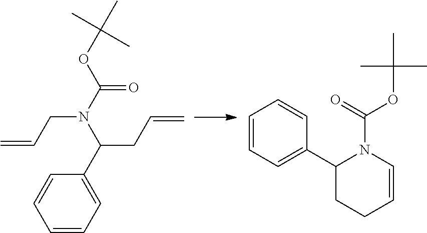 [SMILES: C=CCC(c1ccccc1)N(CC=C)C(=O)OC(C)(C)C ; CC(C)(C)OC(=O)N1C=CCCC1c1ccccc1]

In a sealed tube with a septum, a solution of tert-butyl N-allyl-N-(1-phenylbut-3-enyl)carbamate (404 mg, 1.4057 mmol) in toluene (12 mL) was bubbled through with nitrogen for 10 min. Then Grubbs Catalyst 2^{nd} generation (32.1 mg, 0.0378 mmol) was added and the reaction mixture was heated at 80° C. for 2 h. Then, ground sodium hydroxide (86 mg, 2.1502 mmol) was added to the reaction mixture, the sealed tube was sealed and heated to 110° C. for overnight. Once cooled to room temperature, water (50 mL) was added and the phases were separated. The aqueous layer was extracted with methyl tert-butyl ether (2×50 mL). The combined organic layers were washed with brine, dried over anhydrous sodium sulfate, filtered and concentrated under reduced pressure. The residue was purified on silica gel chromatography, eluting from 0% to 5% ethyl acetate in heptanes to give tert-butyl 2-phenyl-3,4-dihydro-2H-pyridine-1-carboxylate (94 mg, 26%) as a white solid. ESI-MS m/z calc. 259.1572, found 204.2 (M-C₄H₈+1)⁺; Retention time: 2.34 minutes (LC method C) and tert-butyl 2-phenyl-3,6-dihydro-2H-pyridine-1-carboxylate (151 mg, 41%) was also obtained as yellow-brown oil. ESI-MS m/z calc. 259.1572, found 204.2.0 (M-C₄H₈+1)⁺+; Retention time: 2.25 minutes (LC method C).

### Step 5: tert-Butyl 5-hydroxy-2-phenyl-piperidine-1-carboxylate, diastereomer 1 and 2

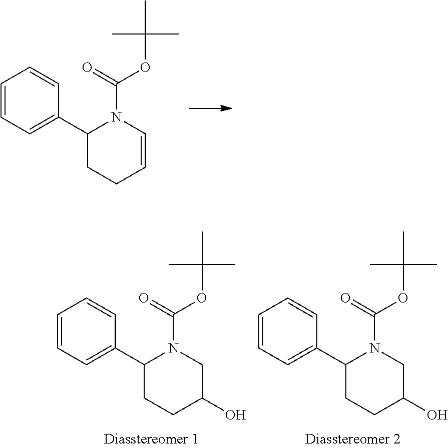 [SMILES: CC(C)(C)OC(=O)N1C=CCCC1c1ccccc1 ; CC(C)(C)OC(=O)N1CC(O)CCC1c1ccccc1 ; CC(C)(C)OC(=O)N1CC(O)CCC1c1ccccc1]

To a solution of tert-butyl 2-phenyl-3,4-dihydro-2H-pyridine-1-carboxylate (94 mg, 0.3625 mmol) in tetrahydrofuran (3 mL), at −78° C., was added borane dimethyl sulfide (40.050 mg, 0.05 mL, 0.5272 mmol). The reaction mixture was stirred at −78° C. for 1 h, then stirred at room temperature overnight. The reaction mixture was cooled to 0° C., aqueous sodium hydroxide solution (0.8 mL of 2 M, 1.6000 mmol) and hydrogen peroxide (0.5 mL of 30% w/v, 4.4099 mmol) were added successively and stirred at room temperature for 1 h. Water (25 mL) was added and extracted with ethyl acetate (2×40 mL). The combined organic layers were washed with brine, dried over anhydrous sodium sulfate, filtered and concentrated under reduced pressure. The residue was purified on silica gel chromatography, eluting from 0% to 30% ethyl acetate in heptanes to give tert-butyl 5-hydroxy-2-phenyl-piperidine-1-carboxylate (24 mg, 24%) as colorless viscous oil (diastereomer 1), ¹H NMR (300 MHz, CDCl₃) δ 1.48 (s, 9H), 1.58-1.80 (m, 3H), 2.13 (dd, J=14.4, 2.9 Hz, 1H), 2.23-2.43 (m, 1H), 2.91 (d, J=14.1 Hz, 1H), 3.87 (br. s., 1H), 4.09 (d, J=13.8 Hz, 1H), 5.44 (br. s., 1H), 7.16-7.44 (m, 5H). ESI-MS m/z calc. 277.1678, found 300.2 (M+Na)⁺; Retention time: 1.91 minutes (LC method C); and tert-butyl 5-hydroxy-2-phenyl-piperidine-1-carboxylate (43 mg, 43%) as colorless viscous oil (diastereomer 2). ¹H NMR (300 MHz, CDCl₃) δ 1.49 (s, 9H), 1.57-1.86 (m, 3H), 2.14 (dq, J=14.3, 3.3 Hz, 1H), 2.24-2.44 (m, 1H), 2.92 (dd, J=14.4, 1.8 Hz, 1H), 3.88 (br. s., 1H), 4.04-4.18 (m, 1H), 5.46 (br. s., 1H), 7.20-7.44 (m, 5H). ESI-MS m/z calc. 277.1678, found 300.2 (M+Na)⁺+; Retention time: 1.90 minutes (LC method C).

### Step 6: 18-(2,6-dimethylphenyl)-6-phenyl-2-oxa-14λ⁶-thia-7,15,17,20-tetraazatetracyclo[14.3.1.13,7.19,13]docosa-1(19),9(21),10,12,16(20),17-hexaene-8,14,14-trione, diastereomer 1 (Compound 225)

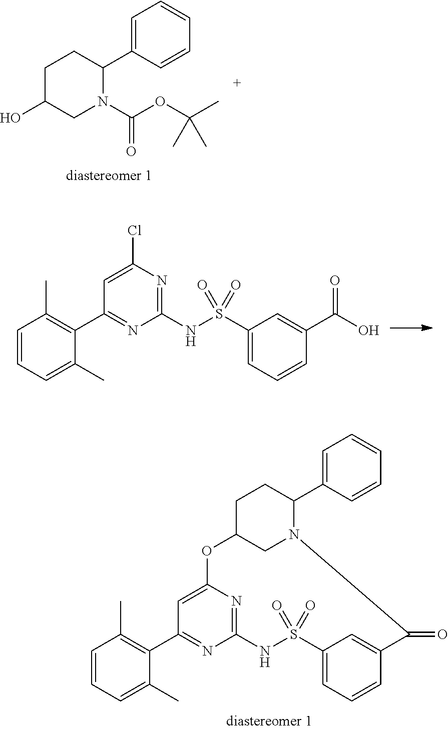 [SMILES: CC(C)(C)OC(=O)N1CC(O)CCC1c1ccccc1 ; Cc1cccc(C)c1-c1cc(Cl)nc(NS(=O)(=O)c2cccc(C(=O)O)c2)n1 ; Cc1cccc(C)c1-c1cc2nc(n1)NS(=O)(=O)c1cccc(c1)C(=O)N1CC(CCC1c1ccccc1)O2]

In a 3-mL vial, tert-butyl 5-hydroxy-2-phenyl-piperidine-1-carboxylate (diastereomer 1) (50 mg, 0.1687 mmol) and 3-[[4-chloro-6-(2,6-dimethylphenyl)pyrimidin-2-yl]sulfamoyl]benzoic acid (70 mg, 0.1675 mmol) were dissolved in NMP (1.0 mL), to which NaH (35 mg of 60% w/w, 0.8751 mmol) was added. This mixture was stirred at room temperature for 2.5 h, after which it was quenched dropwise with 1N HCl (1.0 mL) and extracted with ethyl acetate (3×2 mL). The combined organic extracts were washed with water (4 mL) and saturated aqueous sodium chloride solution (4 mL), then dried over sodium sulfate, filtered, and evaporated in vacuo. The crude product, 3-[[4-[(1-tert-butoxycarbonyl-6-phenyl-3-piperidyl)oxy]-6-(2,6-dimethylphenyl)pyrimidin-2-yl]sulfamoyl]benzoic acid (ca. 150 mg), was taken onto the next step without further purification. In a 3-mL vial, the crude product from above was dissolved in dioxane (0.75 mL) and treated with a dioxane solution of HCl (0.25 mL of 4.0 M, 1.000 mmol). This mixture was stirred at room temperature for 15 min then at 50° C. for 1 h. It was then cooled to room temperature, diluted with 1:1 MeOH:DMSO (1 mL), filtered and purified by reverse phase HPLC (1-50% acetonitrile in water using HCl as a modifier) to give 3-[[4-(2,6-dimethylphenyl)-6-[(6-phenyl-3-piperidyl)oxy]pyrimidin-2-yl]sulfamoyl]benzoic acid (hydrochloride salt) (43.2 mg, 43%) ESI-MS m/z calc. 558.19366, found 559.4 (M+1)⁺; Retention time: 1.17 minutes (LC method A).

In a 3-mL vial, the product from above was dissolved in DMF (0.6 mL), and treated with DIPEA (50 μL, 0.2871 mmol) and Ph₂P(O)—OC₆F₅ (58.2 mg, 0.1515 mmol). This mixture was stirred at room temperature for 10 min, after which it was diluted with MeOH (0.3 mL), filtered and purified by reverse phase HPLC (1-70% acetonitrile in water using HCl as a modifier) to give (2,3,4,5,6-pentafluorophenyl) 3-[[4-(2,6-dimethylphenyl)-6-[(6-phenyl-3-piperidyl)oxy]pyrimidin-2-yl]sulfamoyl]benzoate (26.3 mg, 22%) ESI-MS m/z calc. 724.17786, found 725.4 (M+1)⁺; Retention time: 1.7 minutes (LC method A).

In a 3-mL vial, the product from above was dissolved in NMP (1.0 mL), and heated at 90° C. for 30 min then at 130° C. for 30 min. This mixture was then cooled to room temperature, filtered and purified by reverse phase HPLC (1-70% acetonitrile in water using HCl as a modifier) to give 18-(2,6-dimethylphenyl)-6-phenyl-2-oxa-14λ⁶-thia-7,15,17,20-tetraazatetracyclo[14.3.1.13,7.19,13]docosa-1(19),9(21),10,12,16(20),17-hexaene-8,14,14-trione (6 mg, 7%) ESI-MS m/z calc. 540.1831, found 541.4 (M+1)⁺; Retention time: 1.72 minutes (LC method A).

### Example 123: Preparation of Compound 226

### Step 1: tert-Butyl 5-hydroxy-2-phenyl-piperidine-1-carboxylate diastereomer 1 and 2

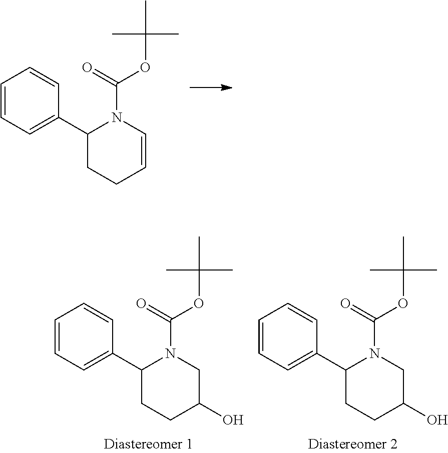 [SMILES: CC(C)(C)OC(=O)N1C=CCCC1c1ccccc1 ; CC(C)(C)OC(=O)N1CC(O)CCC1c1ccccc1 ; CC(C)(C)OC(=O)N1CC(O)CCC1c1ccccc1]

To a solution of tert-butyl 2-phenyl-3,4-dihydro-2H-pyridine-1-carboxylate (1.17 g, 4.5114 mmol) in tetrahydrofuran (35 mL), at −78° C., was added borane dimethyl sulfide (480.60 mg, 0.6 mL, 6.3263 mmol). The reaction mixture was stirred at −78° C. for 1 h, then stirred at room temperature overnight. The reaction mixture was cooled to 0° C., aqueous sodium hydroxide solution (10 mL of 2 M, 20.000 mmol) and hydrogen peroxide (6.5 mL of 30% w/v, 57.328 mmol) were slowly added successively. The reaction mixture was stirred at 0° C. for 5 min, then stirred at room temperature for 1 h. Water (80 mL) was added and extracted with ethyl acetate (2×100 mL). The combined organic layers were washed with brine, dried over anhydrous sodium sulfate, filtered and concentrated under reduced pressure. The residue was purified on silica gel chromatography, eluting from 0% to 30% ethyl acetate in heptanes to give tert-butyl 5-hydroxy-2-phenyl-piperidine-1-carboxylate (314 mg, 25%) as a viscous colorless oil (diastereoisomer 1). ¹H NMR (300 MHz, CDCl₃) δ 1.49 (s, 9H), 1.57-1.86 (m, 3H), 2.14 (dq, J=14.3, 3.3 Hz, 1H), 2.24-2.44 (m, 1H), 2.92 (dd, J=14.4, 1.8 Hz, 1H), 3.88 (br. s., 1H), 4.04-4.18 (m, 1H), 5.46 (br. s., 1H), 7.20-7.44 (m, 5H). ESI-MS m/z calc. 277.1678, found 300.2 (M+Na)⁺+; Retention time: 4.11 minutes (LC method G); and tert-butyl 5-hydroxy-2-phenyl-piperidine-1-carboxylate (119 mg, 9%) was also obtained as colorless viscous oil (diastereomer 2), ¹H NMR (300 MHz, CDCl₃) δ 1.31-1.44 (m, 2H), 1.49 (s, 9H), 1.85-2.02 (m, 2H), 2.35-2.47 (m, 1H), 2.52 (dd, J=12.8, 10.7 Hz, 1H), 3.73 (td, J=10.4, 5.3 Hz, 1H), 4.22 (dd, J=12.9, 5.0 Hz, 1H), 5.40 (br. s., 1H), 7.15-7.42 (m, 5H). ESI-MS m/z calc. 277.1678, found 300.2 (M+Na)⁺+; Retention time: 4.11 minutes (LC method G).

### Step 2: 18-(2,6-Dimethylphenyl)-6-phenyl-2-oxa-14λ⁶-thia-7,15,17,20-tetraazatetracyclo[14.3.1.13,7.19,13]docosa-1(19),9(21),10,12,16(20),17-hexaene-8,14,14-trione, diastereomer 2 (Compound 226)

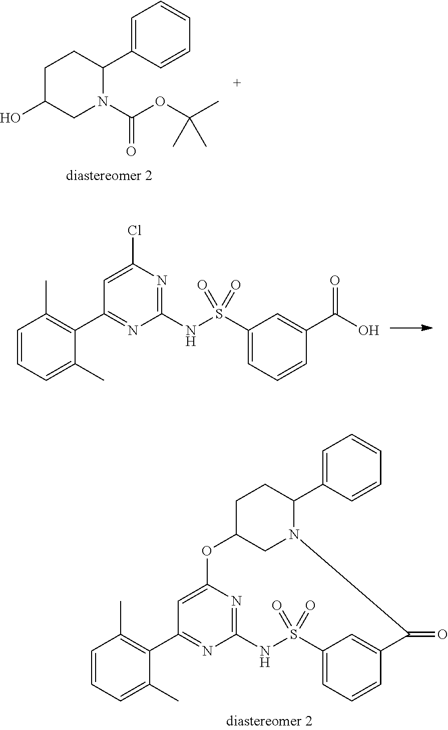 [SMILES: CC(C)(C)OC(=O)N1CC(O)CCC1c1ccccc1 ; Cc1cccc(C)c1-c1cc(Cl)nc(NS(=O)(=O)c2cccc(C(=O)O)c2)n1 ; Cc1cccc(C)c1-c1cc2nc(n1)NS(=O)(=O)c1cccc(c1)C(=O)N1CC(CCC1c1ccccc1)O2]

In a 3-mL vial, tert-butyl 5-hydroxy-2-phenyl-piperidine-1-carboxylate (50 mg, 0.1713 mmol) and 3-[[4-chloro-6-(2,6-dimethylphenyl)pyrimidin-2-yl]sulfamoyl]benzoic acid (70 mg, 0.1675 mmol) were dissolved in NMP (1.0 mL), to which NaH (35 mg of 60% w/w, 0.8751 mmol) was added. This mixture was stirred at room temperature for 2.5 h, after which it was quenched dropwise with 1 N HCl (1.0 mL) and extracted with ethyl acetate (3×2 mL). The combined organic extracts was washed with water (4 mL) and saturated aqueous sodium chloride solution (4 mL), then dried over sodium sulfate, filtered, and evaporated in vacuo. The crude product, 3-[[4-[(1-tert-butoxycarbonyl-6-phenyl-3-piperidyl)oxy]-6-(2,6-dimethylphenyl)pyrimidin-2-yl]sulfamoyl]benzoic acid (ca. 150 mg), was taken onto the next step without further purification. In a 3-mL vial, the crude product was dissolved in dioxane (0.75 mL) and treated with a dioxane solution of HCl (0.25 mL of 4.0 M, 1.000 mmol). This mixture was stirred at room temperature for 15 min then at 50° C. for 1 h. It was then cooled to room temperature, diluted with 1:1 MeOH:DMSO (1 mL), filtered and purified by reverse phase HPLC (1-50% acetonitrile in water using HCl as a modifier) to give 3-[[4-(2,6-dimethylphenyl)-6-[(6-phenyl-3-piperidyl)oxy]pyrimidin-2-yl]sulfamoyl]benzoic acid (hydrochloride salt) (55.7 mg, 56%) ESI-MS m/z calc. 558.19366, found 559.4 (M+1)⁺; Retention time: 1.14 minutes (LC method A).

In a 3-mL vial, the product from above was dissolved in DMF (0.6 mL), and treated with DIPEA (50 μL, 0.2871 mmol) and HATU (50 mg, 0.1315 mmol). This mixture was stirred at room temperature for 5 min, after which it was diluted with MeOH (0.3 mL), filtered and purified by reverse phase HPLC (1-99% acetonitrile in water using HCl as a modifier) to give 18-(2,6-dimethylphenyl)-6-phenyl-2-oxa-14λ⁶-thia-7,15,17,20-tetraazatetracyclo[14.3.1.13,7.19,13]docosa-1(19),9(21),10,12,16(20),17-hexaene-8,14,14-trione (33.1 mg, 37%) ¹H NMR (400 MHz, dimethylsulfoxide-d₆) δ 13.37-11.56 (bs, 1H), 8.63 (s, 1H), 8.02-7.91 (m, 1H), 7.90-7.77 (m, 1H), 7.76-7.64 (m, 1H), 7.46-7.36 (m, 4H), 7.33-7.28 (m, 1H), 7.24 (t, J=7.5 Hz, 1H), 7.10 (d, J=7.6 Hz, 2H), 6.24 (s, 1H), 5.81 (d, J=5.2 Hz, 1H), 5.40-5.28 (m, 1H), 3.80 (d, J=13.3 Hz, 1H), 2.71 (d, J=14.5 Hz, 1H), 2.55 (dd, J=14.5, 10.8 Hz, 1H), 2.41-2.29 (m, 1H), 2.27-2.20 (m, 1H), 2.16-1.87 (bs, 6H), 1.81-1.69 (m, 1H) ESI-MS m/z calc. 540.1831, found 541.4 (M+1)⁺; Retention time: 1.8 minutes (LC method A).

### Example 124: Preparation of Compound 227

### Step 1: 3-[Benzyl-(2,2,2-trifluoroacetyl)amino]propanoic acid

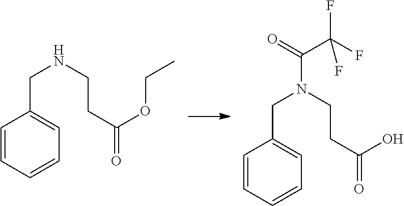 [SMILES: CCOC(=O)CCNCc1ccccc1 ; O=C(O)CCN(Cc1ccccc1)C(=O)C(F)(F)F]

To a solution of ethyl 3-(benzylamino)propanoate (25 g, 120.8 mmol) in ethanol (200 mL) was added potassium hydroxide (8.1 g, 144.6 mmol) and the mixture was stirred at room temperature for 5 hours. The reaction mixture was neutralized with trifluoroacetic acid and concentrated. To the residue trifluoroacetic anhydride (100 mL, 708 mmol) was added and the mixture was stirred at room temperature for 16 hours. The mixture was concentrated, and the residue was partitioned between chloroform (200 mL) and water (200 mL). The organic layer was separated, and the aqueous layer was extracted with chloroform (2×50 mL). The combined organic layer was dried over sodium sulfate and concentrated to give crude 3-(N-benzyl-2,2,2-trifluoroacetamido) propionic acid (30.9 g, 93%) as a colorless oil. ESI-MS m/z calc. 275.07693, found 276.2 (M+1)⁺; Retention time: 2.52 minutes; LC method B.

### Step 2: 2-(2,2,2-Trifluoroacetyl)-3,4-dihydro-1H-2-benzazepin-5-one

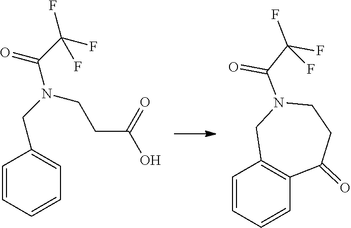 [SMILES: O=C(O)CCN(Cc1ccccc1)C(=O)C(F)(F)F ; O=C1CCN(C(=O)C(F)(F)F)Cc2ccccc21]

A mixture of 3-(N-benzyl-2,2,2-trifluoroacetamido)propionic acid (30.9 g, 112.4 mmol) and thionyl chloride (150 mL, 2.06 mol) was stirred at room temperature for 16 hours and concentrated. The residue was dissolved in 1,2-dichloroethane (350 mL) and anhydrous aluminum chloride (31 g, 233 mmol) was added. The mixture was stirred at 60 C for 1 hour and, then poured into ice-water (500 mL). The organic layer was separated, and the aqueous layer was extracted with chloroform (2×100 mL). The combined organic layer was dried over sodium sulfate, concentrated and the residue was purified by silica gel column chromatography using 0-20% hexanes-ethyl acetate to give 2-(2,2,2-trifluoroacetyl)-1,2,3,4-tetrahydrobenzo[c]azepin-5-one (10.83 g, 37%) as a colorless oil. ESI-MS m/z calc. 257.06638, found 258.3 (M+1)⁺; Retention time: 2.51 minutes; LC method B.

### Step 3: tert-Butyl 5-hydroxy-1,3,4,5-tetrahydro-2-benzazepine-2-carboxylate

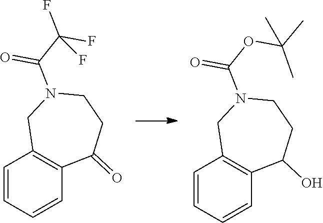 [SMILES: O=C1CCN(C(=O)C(F)(F)F)Cc2ccccc21 ; CC(C)(C)OC(=O)N1CCC(O)c2ccccc2C1]

To a solution of 2-(2,2,2-trifluoroacetyl)-1,2,3,4-tetrahydrobenzo[c]azepin-5-one (10.83 g, 42.1 mmol) in ethanol (200 mL) was added sodium borohydride (3.12 g, 82.1 mmol) portionwise and the mixture was stirred at room temperature for 1 hour. 3M Aqueous potassium carbonate solution (200 mL) and chloroform (200 mL) was added followed by addition di-tert-butyl dicarbonate (10 g, 45.87 mmol). The mixture was stirred at room temperature for 3 hours. The organic layer was separated, and the aqueous layer was extracted with chloroform (2×100 mL). The combined organic layer was dried over sodium sulfate and concentrated. The residue was purified by silica gel column chromatography using 0-20% hexanes-ethyl acetate to give tert-butyl 5-hydroxy-4,5-dihydro-1H-benzo[c]azepine-2(3H)-carboxylate (10.57 g, 95%) as a white solid. ¹H NMR (250 MHz, DMSO) δ 7.41 (d, J=7 Hz, 1H), 7.26-7.16 (m, 3H), 5.42 (d, J=4 Hz, 1H), 4.87 (br. s, 1H), 4.51 (d, J=14.75 Hz, 1H), 4.24 (d, J=14.75 Hz, 1H), 3.89-3.73 (m, 1H), 3.65-3.43 (m, 1H), 1.90-1.59 (m, 2H), 1.29 (s, 9H). ESI-MS m/z calc. 263.15213, found 264.0 (M+1)⁺; Retention time: 2.03 minutes; LC method B.

### Step 4: 12-(2,6-dimethylphenyl)-15-oxa-8λ⁶-thia-1,9,11,26-tetraazapentacyclo[14.7.2.13,7.110,14.017,22]heptacosa-3(27),4,6,10(26),11,13,17,19,21-nonaene-2,8,8-trione (Compound 227)

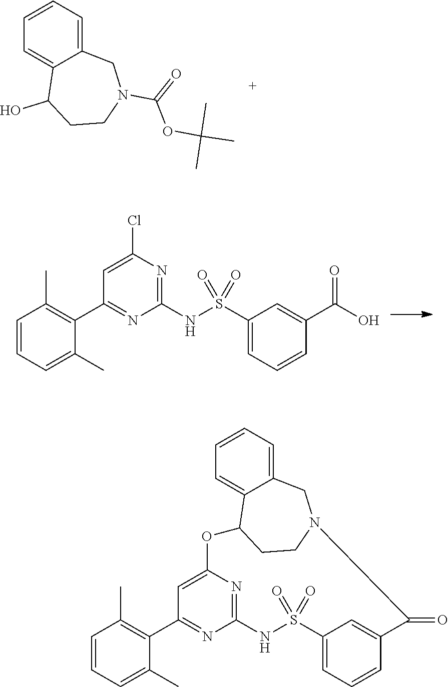 [SMILES: CC(C)(C)OC(=O)N1CCC(O)c2ccccc2C1 ; Cc1cccc(C)c1-c1cc(Cl)nc(NS(=O)(=O)c2cccc(C(=O)O)c2)n1 ; Cc1cccc(C)c1-c1cc2nc(n1)NS(=O)(=O)c1cccc(c1)C(=O)N1CCC(O2)c2ccccc2C1]

In a 3-mL vial, tert-butyl 5-hydroxy-1,3,4,5-tetrahydro-2-benzazepine-2-carboxylate (60 mg, 0.2228 mmol) and 3-[[4-chloro-6-(2,6-dimethylphenyl)pyrimidin-2-yl]sulfamoyl]benzoic acid (80 mg, 0.1914 mmol) were dissolved in NMP (1.0 mL), to which NaH (35 mg of 60% w/w, 0.8751 mmol) was added. This mixture was stirred at room temperature for 1.5 h, after which it was quenched dropwise with 1N HCl (1.0 mL) and extracted with ethyl acetate (3×2 mL). The combined organic extracts were washed with water (4 mL) and saturated aqueous sodium chloride solution (4 mL), then dried over sodium sulfate, filtered, and evaporated in vacuo. The crude product (ca. 160 mg) was taken onto the next step without further purification. In a 3-mL vial, the crude product was dissolved in dioxane (1.5 mL) and treated with a dioxane solution of HCl (0.5 mL of 4.0 M, 2.000 mmol). This mixture was stirred at room temperature for 15 min then at 50° C. for 1.5 h. It was then cooled to room temperature, diluted with 1:1 MeOH:DMSO (1 mL), filtered and purified by reverse phase HPLC (1-50% acetonitrile in water using HCl as a modifier) to give 3-[[4-(2,6-dimethylphenyl)-6-(2,3,4,5-tetrahydro-1H-2-benzazepin-5-yloxy)pyrimidin-2-yl]sulfamoyl]benzoic acid (hydrochloride salt) (49.2 mg, 41%) ESI-MS m/z calc. 544.17804, found 545.4 (M+1)⁺; Retention time: 1.08 minutes (LC method A).

In a 20-mL vial, the product from above (49.2 mg, 93% purity, 0.0787 mmol) was dissolved in DMF (6.0 mL), and treated with DIPEA (0.3 mL, 1.722 mmol) and HATU (75 mg, 0.1972 mmol). This mixture was stirred at room temperature for 5 min, after which it was diluted with MeOH (1.0 mL), filtered and purified by reverse phase HPLC (1-70% acetonitrile in water using HCl as a modifier) to give 12-(2,6-dimethylphenyl)-15-oxa-8λ⁶-thia-1,9,11,26-tetraazapentacyclo[14.7.2.13,7.110,14.017,22]heptacosa-3(27),4,6,10(26),11,13,17,19,21-nonaene-2,8,8-trione (12.2 mg, 12%)¹H NMR (400 MHz, dimethylsulfoxide-d₆) δ 13.42-11.57 (bs, 1H), 8.25-8.03 (bs, 1H), 7.96-7.85 (m, 1H), 7.82-7.64 (m, 2H), 7.62-7.52 (m, 1H), 7.47-7.41 (m, 1H), 7.39-7.32 (m, 2H), 7.28 (t, J=7.5 Hz, 1H), 7.15 (d, J=7.6 Hz, 2H), 6.77 (d, J 10.7 Hz, 1H), 6.73-6.47 (bs, 1H), 5.49 (d, J=16.4 Hz, 1H), 4.47 (d, J=16.3 Hz, 1H), 3.41-3.29 (m, 2H, hidden under the water peak), 2.30-1.91 (m, 8H) ESI-MS m/z calc. 526.1675, found 527.4 (M+1)⁺; Retention time: 1.65 minutes (LC method A).

### Example 125: Preparation of Compound 228

### Step 1: 3-(4-Methoxyphenoxy)-4,5-dihydro-1H-benzo[b]azepin-2(3H)-one

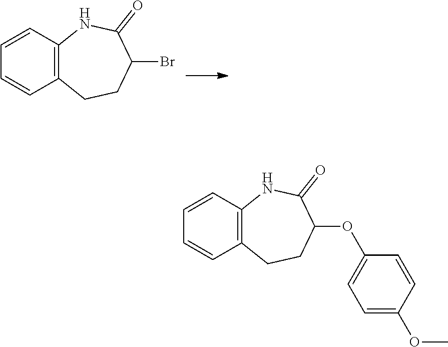 [SMILES: O=C1Nc2ccccc2CCC1Br ; COc1ccc(OC2CCc3ccccc3NC2=O)cc1]

A mixture of 3-bromo-4,5-dihydro-1H-benzo[b]azepin-2(3H)-one (20.0 g, 83.7 mmol) and 4-methoxyphenol (25.95 g, 209.2 mmol) in acetone (349 mL) was cooled to 0° C. Cesium carbonate (68.16 g, 209.2 mmol) was added in one portion and the reaction was stirred at 0° C. for five hours, then allowed to warm to room temperature and stirred for 16 hours. The inorganic solids were filtered off and the filter cake was rinsed with acetone. The solids were dissolved in water (50 mL) and extracted with ethyl acetate (3×75 mL). The combined organic layers were concentrated and dissolved in ethyl acetate (150 mL). he solution was washed with water (50 mL), 1 M aqueous sodium hydroxide solution (50 mL), and brine (50 mL), then dried over sodium sulfate and concentrated to give 3-(4-methoxyphenoxy)-4,5-dihydro-1H-benzo[b]azepin-2(3H)-one (21.1 g, 90%) as a white solid. ESI-MS m/z calc. 283.12, found 284.5 (M+1)⁺. Retention time: 2.74 minutes. ¹H NMR (250 MHz, CDCl₃) δ 7.60 (s, 1H) 7.10-7.33 (m, 3H) 6.93-7.03 (m, 1H) 6.66-6.83 (m, 4H) 4.63 (t, J=8.46 Hz, 1H) 3.72 (s, 3H) 2.91-3.08 (m, 1H) 2.62-2.88 (m, 2H) 2.42-2.59 (m, 1H).

### Step 2: 3-(4-Methoxyphenoxy)-2,3,4,5-tetrahydro-1H-benzo[b]azepine

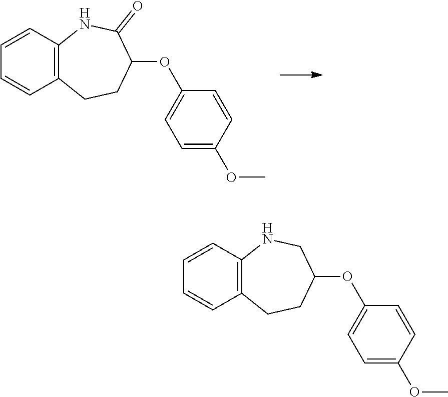 [SMILES: COc1ccc(OC2CCc3ccccc3NC2=O)cc1 ; COc1ccc(OC2CCc3ccccc3NC2)cc1]

To a solution of 3-(4-methoxyphenoxy)-4,5-dihydro-1H-benzo[b]azepin-2(3H)-one (24.03 g, 83.7 mmol) in tetrahydrofuran (175 mL) was added carefully 2 M borane dimethyl sulfide complex in tetrahydrofuran (87.88 mL, 175.76 mmol). Once the bubbling had ceased, the reaction was heated to reflux for 3.5 hours. The reaction was cooled to 0° C. and quenched carefully with water (100 mL). The volatiles were removed under vacuum and the aqueous layer was extracted with ethyl acetate (3×100 mL). The combined organic layers were dried over sodium sulfate and concentrated. The residue was triturated with ethanol and the formed solid was collected by filtration to give 3-(4-methoxyphenoxy)-2,3,4,5-tetrahydro-1H-benzo[b]azepine (15.55 g, 69% over two steps) as a white crystalline powder. ESI-MS m/z calc. 269.14, found 270.3 (M+1)⁺. Retention time: 2.18 minutes. ¹H NMR (250 MHz, CDCl₃) δ7.02-7.15 (m, 2H) 6.80-6.96 (m, 5H) 6.74 (d, J=7.80 Hz, 1H) 4.40 (tt, J=7.95, 3.80 Hz, 1H) 3.79 (s, 3H) 3.37-3.55 (m, 1H) 2.89-3.19 (m, 2H) 2.70 (dd, J=14.23, 11.26 Hz, 2H) 2.08-2.31 (m, 1H) 1.71-1.94 (m, 1H).

### Step 3: Benzyl 3-(4-methoxyphenoxy)-2,3,4,5-tetrahydro-1H-benzo[b]azepine-1-carboxylate

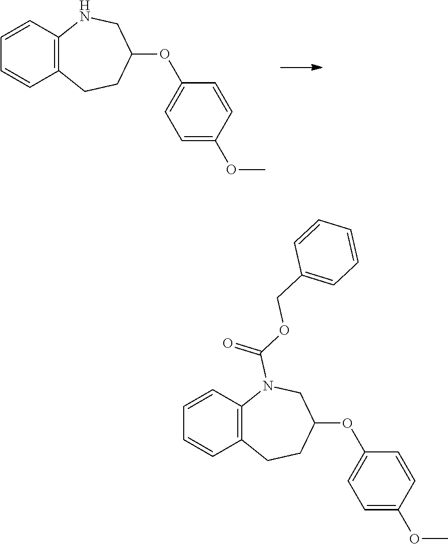 [SMILES: COc1ccc(OC2CCc3ccccc3NC2)cc1 ; COc1ccc(OC2CCc3ccccc3N(C(=O)OCc3ccccc3)C2)cc1]

To a mixture of 3-(4-methoxyphenoxy)-2,3,4,5-tetrahydro-1H-benzo[b]azepine (11.17 g, 41.5 mmol) in tetrahydrofuran (170 mL) and aqueous saturated sodium carbonate (170 mL) was added slowly a 33% benzyl chloroformate solution in toluene (250 mL, 81.74 mmol) and the biphasic mixture was stirred at room temperature for one hour. The layers were separated, and the aqueous layer was extracted with ethyl acetate (3×75 mL). The combined organic layers were washed with water (50 mL), brine (50 mL), dried over sodium sulfate and reduced to give benzyl 3-(4-methoxyphenoxy)-2,3,4,5-tetrahydro-1H-benzo[b]azepine-1-carboxylate as a yellow oil, which was used without further purification. ESI-MS m/z calc. 403.18, found 404.5 (M+1)⁺. Retention time: 3.95 minutes.

### Step 4: Benzyl 3-hydroxy-2,3,4,5-tetrahydro-1H-benzo[b]azepine-1-carboxylate

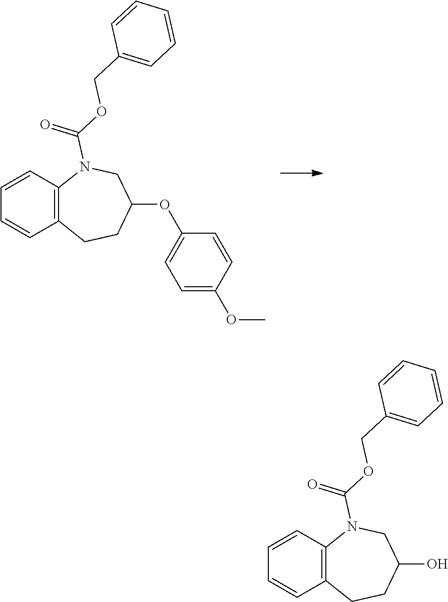 [SMILES: COc1ccc(OC2CCc3ccccc3N(C(=O)OCc3ccccc3)C2)cc1 ; O=C(OCc1ccccc1)N1CC(O)CCc2ccccc21]

A solution of benzyl 3-(4-methoxyphenoxy)-2,3,4,5-tetrahydro-1H-benzo[b]azepine-1-carboxylate (16.73 g, 41.5 mmol) in a 4:1 mixture of acetonitrile (140 mL) and water (35 mL) was cooled to 0° C. Ceric ammonium nitrate (63.7 g, 116.2 mmol) was added portionwise and the reaction mixture was stirred at 0° C. for 10 minutes. The mixture was concentrated, and the residue was dissolved in water (75 mL) and ethyl acetate (75 mL). The layers were separated, and the aqueous layer was extracted with ethyl acetate (3×50 mL). The combined organic layers were washed with water (50 mL), brine (50 mL), dried over sodium sulfate and concentrated. The crude residue was purified by silica gel column chromatography using 0-30% dichloromethane-ethyl acetate to give benzyl 3-hydroxy-2,3,4,5-tetrahydro-1H-benzo[b]azepine-1-carboxylate (6.4 g, 52% over two steps) as an orange oil. ¹H NMR (250 MHz, DMSO) δ 7.84-6.59 (m, 9H), 5.11 (dd, J=24.0, 11.6 Hz, 3H), 4.32 (d, J=13.3 Hz, 1H), 3.72 (s, 1H), 2.86-2.52 (m, 2H), 2.36 (d, J=12.4 Hz, 1H), 2.09 (s, 1H), 1.48-0.89 (m, 1H). ESI-MS m/z calc. 297.14, found 298.6 (M+1)⁺. Retention time: 2.84 minutes.

### Step 5: 2,3,4,5-Tetrahydro-1H-1-benzazepin-3-ol

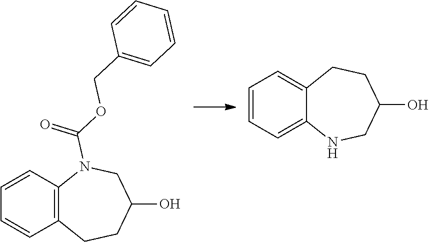 [SMILES: O=C(OCc1ccccc1)N1CC(O)CCc2ccccc21 ; OC1CCc2ccccc2NC1]

To a stirring solution of benzyl 3-hydroxy-2,3,4,5-tetrahydro-1-benzazepine-1-carboxylate (940 mg, 3.1613 mmol) in ethanol (35 mL) at room temperature was added palladium on carbon (220 mg, 10% w/w, 0.2067 mmol) and the reaction mixture was stirred under the atmosphere of hydrogen (1 atm) for 16 hours. The reaction mixture was filtered through a pad of Celite and concentrated under vacuum to afford 2,3,4,5-tetrahydro-1H-1-benzazepin-3-ol (535 mg, 99%) as red oil. The product was carried to the next step without further purification. ESI-MS m/z calc. 163.09972, found 164.6 (M+1)⁺; Retention time: 1.48 minutes; LC method S.

### Step 6: N-[4-Chloro-6-(2,6-dimethylphenyl)pyrimidin-2-yl]-3-(3-hydroxy-2,3,4,5-tetrahydro-1-benzazepine-1-carbonyl)benzenesulfonamide

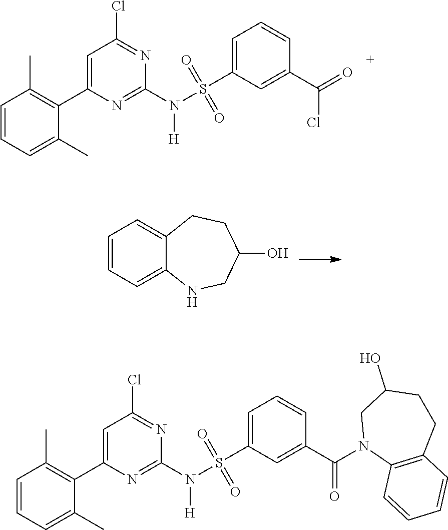 [SMILES: Cc1cccc(C)c1-c1cc(Cl)nc(NS(=O)(=O)c2cccc(C(=O)Cl)c2)n1 ; OC1CCc2ccccc2NC1 ; Cc1cccc(C)c1-c1cc(Cl)nc(NS(=O)(=O)c2cccc(C(=O)N3CC(O)CCc4ccccc43)c2)n1]

To a stirring solution of 2,3,4,5-tetrahydro-1H-1-benzazepin-3-ol (515 mg, 2.9976 mmol) and TEA (1.8150 g, 2.5 mL, 17.937 mmol) in DCM (20 mL) at room temperature was dropwise added a solution of 3-[[4-chloro-6-(2,6-dimethylphenyl)pyrimidin-2-yl]sulfamoyl]benzoyl chloride (1.1 g, 2.1430 mmol) in DCM (20 mL). After the addition was complete, the reaction mixture was stirred for 1 hour. After completion, the reaction was quenched with 1 M aqueous hydrochloric acid (10 mL). Two layers were separated, and the aqueous layer was extracted with DCM (2×25 mL). The combined organic layers were washed with brine (20 mL), dried over anhydrous sodium sulfate and concentrated. The product was purified by silica gel chromatography using 0-65% hexanes-ethyl acetate to afford N-[4-chloro-6-(2,6-dimethylphenyl)pyrimidin-2-yl]-3-(3-hydroxy-2,3,4,5-tetrahydro-1-benzazepine-1-carbonyl)benzenesulfonamide (440 mg, 27%) as white solid. ESI-MS m/z calc. 562.14417, found 563.4 (M+1)⁺; Retention time: 5.51 minutes (LC method S).

### Step 7: 5-(2,6-Dimethylphenyl)-2-oxa-9λ⁶-thia-6,8,16,27-tetraazapentacyclo[14.8.1.13,7.110,14.017,22]heptacosa-3(27),4,6,10,12,14(26),17(22),18,20-nonaene-9,9,15-trione (Compound 228)

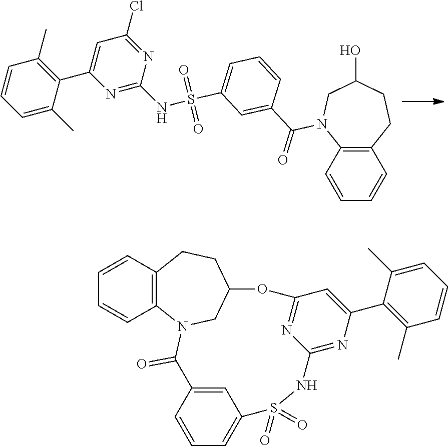 [SMILES: Cc1cccc(C)c1-c1cc(Cl)nc(NS(=O)(=O)c2cccc(C(=O)N3CC(O)CCc4ccccc43)c2)n1 ; Cc1cccc(C)c1-c1cc2nc(n1)NS(=O)(=O)c1cccc(c1)C(=O)N1CC(CCc3ccccc31)O2]

To a stirring solution of N-[4-chloro-6-(2,6-dimethylphenyl)pyrimidin-2-yl]-3-(3-hydroxy-2,3,4,5-tetrahydro-1-benzazepine-1-carbonyl)benzenesulfonamide (370 mg, 0.6571 mmol) in anhydrous DMF (30 mL) at room temperature under nitrogen was added sodium hydride (320 mg, 60% w/w, 8.0008 mmol) and the reaction mixture was stirred for 48 hours. After completion, the reaction mixture was cooled to 0° C. and quenched with 10% aqueous citric acid (25 mL) and water (50 mL). The product was extracted with ethyl acetate (3×50 mL). The combined organic layers were washed with brine (2×40 mL), dried over anhydrous sodium sulfate and concentrated. The product was purified by silica gel chromatography using 0-65% hexanes-ethyl acetate, followed by reverse phase HPLC using water (5 mM HCl buffer)-acetonitrile gradient method (Cis Higgins Analytical column, 35-70% acetonitrile, 25 mL/min) to afford 5-(2,6-dimethylphenyl)-2-oxa-9λ⁶-thia-6,8,16,27-tetraazapentacyclo[14.8.1.13,7.110,14.017,22]heptacosa-3(27),4,6,10,12,14(26),17(22),18,20-nonaene-9,9,15-trione (52 mg, 15%) as white solid. ESI-MS m/z calc. 526.1675, found 527.2 (M+1)⁺; Retention time: 2.34 minutes; LC method T. ¹H NMR (250 MHz, DMSO-d₆) δ 8.68 (s, 1H), 7.95 (d, J=7.6 Hz, 1H), 7.87-7.63 (m, 2H), 7.54-7.19 (m, 6H), 7.12 (d, J=7.6 Hz, 2H), 6.26 (s, 1H), 5.89-5.55 (m, 1H), 3.83 (d, J=13.4 Hz, 1H), 3.12 (t, J=13.5 Hz, 2H), 2.87 (t, J=12.6 Hz, 2H), 2.23-1.88 (m, 6H), 1.59 (q, J=12.3 Hz, 1H).

### Example 126: Preparation of Compound 229

### Step 1: N-[(1-Allylcyclopentyl)methyl]-4-nitro-benzenesulfonamide

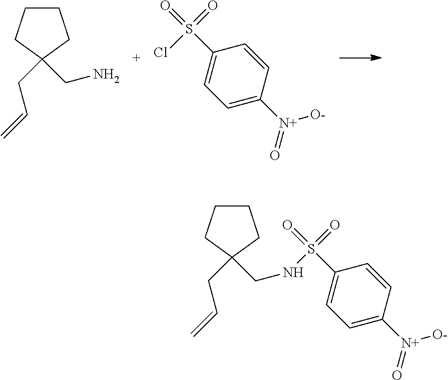

A solution of (1-allylcyclopentyl)methanamine (0.20 g, 1.436 mmol), triethylamine (0.40 mL, 2.870 mmol), and 4-nitrobenzenesulfonyl chloride (0.32 g, 1.444 mmol) in dichloromethane (8 mL) was stirred for 18 hours. The reaction was diluted with dichloromethane and sequentially washed with water, 1 M HCl, and water. The organic solution was dried over sodium sulfate and evaporated. The residue was purified by silica gel column chromatography with 0-30% ethyl acetate in hexanes to give N-[(1-allylcyclopentyl)methyl]-4-nitro-benzenesulfonamide (0.45 g, 97%). ESI-MS m/z calc. 324.11438, found 325.1 (M+1)⁺; Retention time: 0.69 minutes; LC method D. ¹H NMR (400 MHz, Chloroform-d) δ 8.37 (d, J=8.9 Hz, 2H), 8.04 (d, J=8.9 Hz, 2H), 5.82-5.62 (m, 1H), 5.09-5.03 (m, 1H), 5.02-4.99 (m, 1H), 4.77 (t, J=6.5 Hz, 1H), 2.83 (d, J=6.5 Hz, 2H), 2.09 (d, J=7.4 Hz, 2H), 1.69-1.52 (m, 4H), 1.50-1.32 (m, 4H).

### Step 2: [7-(4-Nitrophenyl)sulfonyl-7-azaspiro[4.5]decan-9-yl]2,2,2-trifluoroacetate

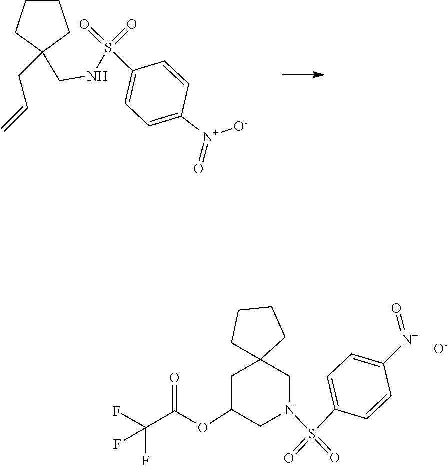

A solution of N-[(1-allylcyclopentyl)methyl]-4-nitro-benzenesulfonamide (0.13 g, 0.4007 mmol), (acetyloxy)(phenyl)-λ³-iodanyl acetate (0.16 g, 0.4967 mmol), and TFA (0.37 mL, 4.803 mmol) in dichloromethane (4 mL) was stirred for 18 hours. The reaction was diluted with dichloromethane and made basic with saturated aqueous sodium bicarbonate. The organics were separated, and the aqueous layer was further extracted with dichloromethane. The combined extracts were washed with water, dried over sodium sulfate, and evaporated. The residue was purified by silica gel column chromatography with 0-30% ethyl acetate in hexanes to give mostly [7-(4-nitrophenyl)sulfonyl-7-azaspiro[4.5]decan-9-yl]2,2,2-trifluoroacetate (0.13 g, 74%) with some hydrolyzed product already present. This was stirred in methanol (4 mL) with potassium carbonate (0.11 g, 0.7959 mmol) for an hour, and the solution was passed through a plug of silica gel, eluting with ethyl acetate. The solution was evaporated under vacuum to give 7-(4-nitrophenyl)sulfonyl-7-azaspiro[4.5]decan-9-ol (0.10 g, 73%) ESI-MS m/z calc. 340.10928, found 341.1 (M+1)⁺; Retention time: 0.57 minutes as a colorless solid (LC method A).

### Step 3: 7-Azaspiro[4.5]decan-9-ol

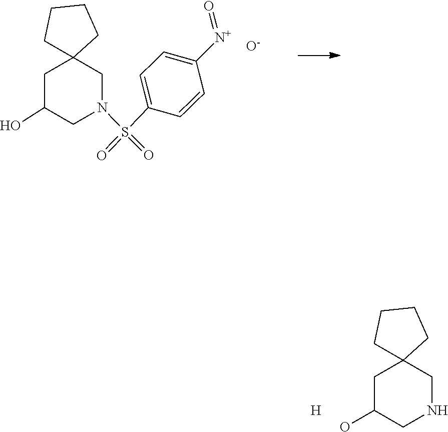

A solution of 7-(4-nitrophenyl)sulfonyl-7-azaspiro[4.5]decan-9-ol (0.16 g, 0.4700 mmol), mercaptoacetic acid (82 μL, 1.179 mmol), and potassium carbonate (0.33 g, 2.388 mmol) in methanol (5 mL) was stirred for 17 hours. The reaction was diluted with water and extracted with ethyl acetate. The combined extracts were dried over sodium sulfate and evaporated. To the residue were added dichloromethane (5 mL), tert-butoxycarbonyl tert-butyl carbonate (0.13 g, 0.5957 mmol), and triethylamine (0.13 mL, 0.9327 mmol), and the reaction was stirred for 19 hours. The reaction was washed with water, dried over sodium sulfate, and evaporated under vacuum. The residue was purified using a reverse phase HPLC-MS method using a Luna C₁₈ (2) column (75×30 mm, 5 μm particle size) sold by Phenomenex (pn: 00C-4252-U0-AX), and a dual gradient run from 1-99% mobile phase B over 15.0 minutes. Mobile phase A=H₂O (5 mM HCl). Mobile phase B=CH₃CN. Flow rate=50 mL/min, and column temperature=25° C. to give tert-butyl 9-hydroxy-7-azaspiro[4.5]decane-7-carboxylate (21 mg, 18%) ESI-MS m/z calc. 255.18344, found 256.2 (M+1)⁺; Retention time: 0.6 minutes, obtained as a colorless oil (LC method D).

### Step 4: 3-[[4-(7-Azaspiro[4.5]decan-9-yloxy)-6-(2,6-dimethylphenyl)pyrimidin-2-yl]sulfamoyl]benzoic acid

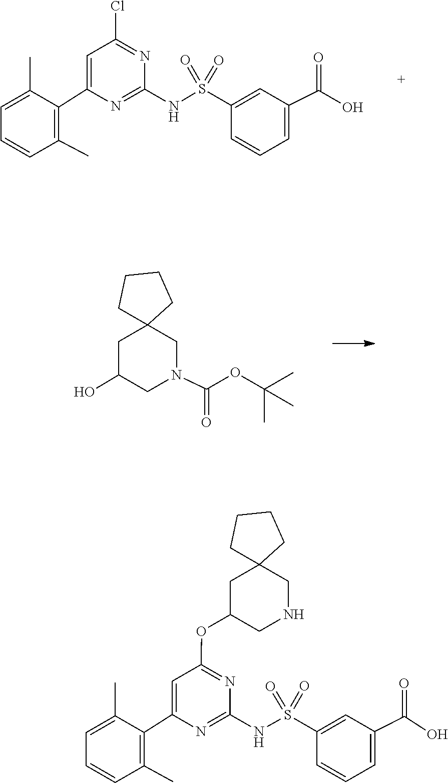 [SMILES: Cc1cccc(C)c1-c1cc(Cl)nc(NS(=O)(=O)c2cccc(C(=O)O)c2)n1 ; CC(C)(C)OC(=O)N1CC(O)CC2(CCCC2)C1 ; Cc1cccc(C)c1-c1cc(OC2CNCC3(CCCC3)C2)nc(NS(=O)(=O)c2cccc(C(=O)O)c2)n1]

A solution of 3-[[4-chloro-6-(2,6-dimethylphenyl)pyrimidin-2-yl]sulfamoyl]benzoic acid (35 mg, 0.08376 mmol), tert-butyl 9-hydroxy-7-azaspiro[4.5]decane-7-carboxylate (21 mg, 0.08224 mmol), and sodium t-butoxide (33 mg, 0.3434 mmol) in THE (1 mL) was stirred for 21 hours. The reaction was quenched with 1 M citric acid, diluted with water, and extracted with ethyl acetate. The combined extracts were washed with brine, dried over sodium sulfate, and evaporated. The residue was purified using a reverse phase HPLC-MS method using a Luna C₁₈ (2) column (75×30 mm, 5 μm particle size) sold by Phenomenex (pn: 00C-4252-U0-AX), and a dual gradient run from 1-99% mobile phase B over 15.0 minutes. Mobile phase A=H₂O (5 mM HCl). Mobile phase B=CH₃CN. Flow rate=50 mL/min, and column temperature=25° C. The resulting colorless solid was stirred with HCl (2 mL of 4 M, 8.000 mmol) (in dioxane) for an hour, and the solvent was evaporated to give as a colorless solid 3-[[4-(7-azaspiro[4.5]decan-9-yloxy)-6-(2,6-dimethylphenyl)pyrimidin-2-yl]sulfamoyl]benzoic acid (hydrochloride salt) (28 mg, 59%) ESI-MS m/z calc. 536.20935, found 537.3 (M+1)⁺; Retention time: 0.45 minutes (LC method D).

### Step 5: 18′-(2,6-Dimethylphenyl)-2′-oxa-14′λ⁶-thia-7′,15′,17′,20′-tetraazaspiro[cyclopentane-1,5′-tetracyclo[14.3.1.13,7.19,13]docosane]-1′(20′),9′,11′,13′(21′),16′,18′-hexaene-8′,14′,14′-trione (Compound 229)

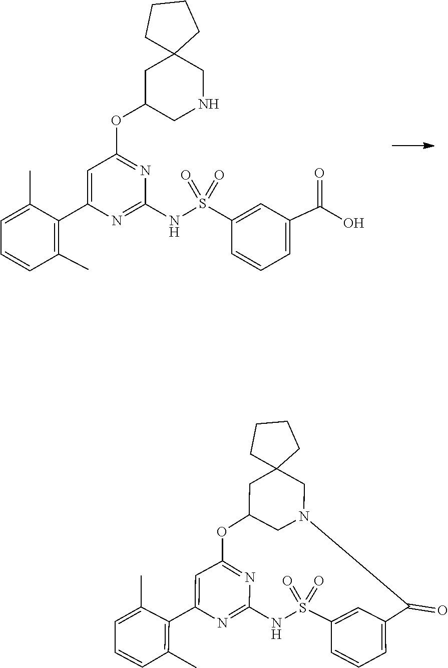 [SMILES: Cc1cccc(C)c1-c1cc(OC2CNCC3(CCCC3)C2)nc(NS(=O)(=O)c2cccc(C(=O)O)c2)n1 ; Cc1cccc(C)c1-c1cc2nc(n1)NS(=O)(=O)c1cccc(c1)C(=O)N1CC(CC3(CCCC3)C1)O2]

A solution of 3-[[4-(7-azaspiro[4.5]decan-9-yloxy)-6-(2,6-dimethylphenyl)pyrimidin-2-yl]sulfamoyl]benzoic acid (hydrochloride salt) (28 mg, 0.04886 mmol), HATU (23 mg, 0.06049 mmol), and triethylamine (28 μL, 0.2009 mmol) in DMF (3 mL) was stirred for 17 hours. The reaction was diluted with water, acidified with 1 M citric acid, and extracted with ethyl acetate. The combined extracts were washed with brine, dried over sodium sulfate, and evaporated under vacuum. The residue was purified using a reverse phase HPLC-MS method using a Luna C₁₈ (2) column (75×30 mm, 5 μm particle size) sold by Phenomenex (pn: 00C-4252-U0-AX), and a dual gradient run from 1-99% mobile phase B over 15.0 minutes. Mobile phase A=H₂O (5 mM HCl). Mobile phase B=CH₃CN. Flow rate=50 mL/min, and column temperature=25° C. to give 18′-(2,6-dimethylphenyl)-2′-oxa-14′λ⁶-thia-7′,15′,17,20′-tetraazaspiro[cyclopentane-1,5′-tetracyclo[14.3.1.13,7.19,13]docosane]-1′(20′),9′,11′,13′(21′),16′,18′-hexaene-8′,14′,14′-trione (13 mg, 47%), obtained as a light-yellow solid. ESI-MS m/z calc. 518.1988, found 519.3 (M+1)⁺; Retention time: 1.69 minutes; LC method A. ¹H NMR (400 MHz, DMSO-d₆) δ 8.39 (s, 1H), 7.93 (s, 1H), 7.68 (s, 2H), 7.32-7.21 (m, 1H), 7.18-7.07 (m, 2H), 6.36 (s, 1H), 5.22 (s, 1H), 4.25 (d, J=12.6 Hz, 1H), 3.93-3.80 (m, 1H), 2.81 (dd, J=13.0, 10.7 Hz, 1H), 2.65 (d, J=12.6 Hz, 1H), 2.16-1.99 (m, 7H), 1.93 (t, J=11.9 Hz, 1H), 1.76-1.47 (m, 8H),

### Example 127: Preparation of Compound 230

### Step 1: 7-Methyloct-4-enoic acid (E/Z mixture)

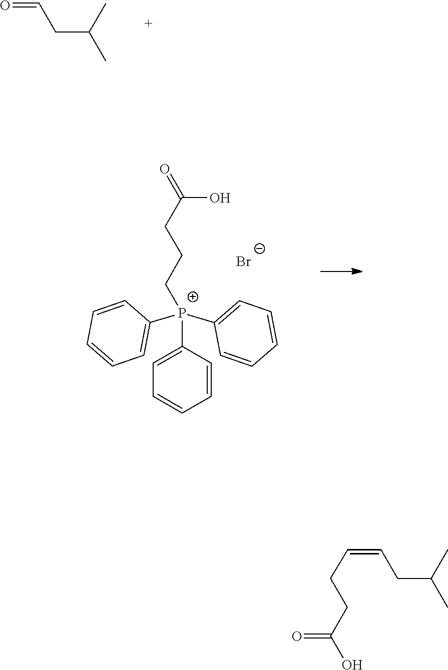

A suspension of (3-carboxypropyl)triphenylphosphoium bromide (47.222 g, 110.00 mmol) in anhydrous THE (10 mL) under nitrogen was cooled to −10° C. and LiHMDS (220.00 mL of 1 M, 220.00 mmol) was added dropwise. The reaction mixture was stirred for 30 minutes and then cooled to −78° C. 3-Methylbutanal (8.6132 g, 100 mmol) was added dropwise and the reaction mixture was allowed to slowly warm up to room temperature overnight. The reaction was quenched with water (500 mL) and the volatiles were removed under vacuum. The residual aqueous layer was washed with diethyl ether (2×250 mL), acidified with aqueous hydrochloric acid (1 M) to pH ˜2. and the product was extracted with ethyl acetate (3×250 mL). Combined organic layers were washed with brine (150 mL), dried over anhydrous sodium sulfate and concentrated to afford a mixture of Z- and E-isomers of 7-methyloct-4-enoic acid (13.93 g, 89%) as amber oil. The product was taken forward to the next step without further purification. ¹H NMR (250 MHz, CDCl₃) δ 5.58-5.28 (m, 2H), 2.50-2.21 (m, 4H), 1.90 (dt, J=18.1, 6.4 Hz, 2H), 1.59 (dq, J=13.5, 7.0 Hz, 1H), 0.87 (dd, J=8.2, 6.6 Hz, 6H).

### Step 2: 5-(1-Hydroxy-3-methyl-butyl)tetrahydrofuran-2-one, diastereomer 1 and 2

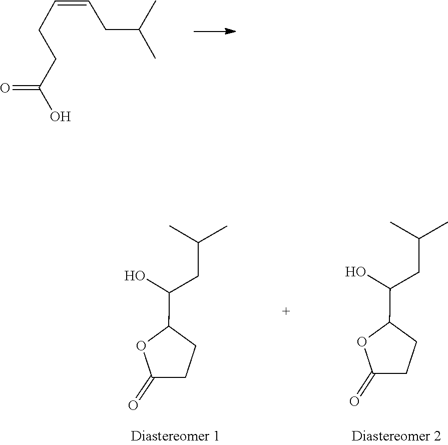

To a stirring suspension of methylrhenium(VII) trioxide (1.3068 g, 5.2431 mmol) in chloroform (100 mL) at room temperature was added aqueous hydrogen peroxide (8.2071 g, 50% w/w, 120.64 mmol), followed by the addition of a solution of (Z/E)-7-methyloct-4-enoic acid (13.93 g, 89.168 mmol) in acetonitrile (100 mL). The obtained reaction mixture was stirred for 18 hours. After completion, the reaction was quenched with 1 M aqueous sodium carbonate (30 mL) and water (30 mL). Volatiles were removed under vacuum and the product was extracted with ethyl acetate (3×100 mL). Combined organic layers were washed with brine (40 mL), dried over anhydrous sodium sulfate and concentrated. The crude product was purified by silica gel chromatography using 0-30% hexanes-ethyl acetate to afford 2 isomers: diastereomer 1 (7.29 g, 47.5%) as white solid and diastereomer 2 (4.02 g, 26.2%) as yellow oil. Diastereomer: ¹H NMR (250 MHz, CDCl₃) δ 4.38 (td, J=7.4, 4.5 Hz, 1H), 3.65 (dt, J=10.0, 4.1 Hz, 1H), 2.68-2.43 (m, 2H), 2.33-2.01 (m, 2H), 1.98-1.76 (m, 2H), 1.53 (ddd, J=14.5, 10.0, 4.9 Hz, 1H), 1.23 (ddd, J=13.4, 7.2, 3.4 Hz, 1H), 0.94 (td, J=6.5, 2.0 Hz, 6H). Diastereomer 2: ¹H NMR (250 MHz, CDCl₃) δ 4.41 (td, J=7.3, 3.0 Hz, 1H), 4.03 (dt, J=9.8, 3.3 Hz, 1H), 2.69-2.40 (m, 2H), 2.38-1.98 (m, 3H), 1.82 (dddd, J=13.3, 11.6, 9.0, 6.6 Hz, 1H), 1.38 (ddd, J=14.6, 9.8, 5.0 Hz, 1H), 1.14 (ddd, J=13.8, 9.1, 3.6 Hz, 1H), 0.94 (dt, J=9.1, 5.5 Hz, 6H).

### Step 3: [3-Methyl-1-(5-oxotetrahydrofuran-2-yl)butyl]methanesulfonate, diastereomer 2

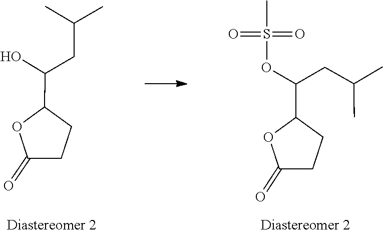 [SMILES: CC(C)CC(O)C1CCC(=O)O1 ; CC(C)CC(OS(C)(=O)=O)C1CCC(=O)O1]

To a stirring solution of 5-(1-hydroxy-3-methyl-butyl)tetrahydrofuran-2-one, diastereomer 2 (4.02 g, 23.342 mmol) and triethylamine (3.5430 g, 35.013 mmol) in anhydrous DCM (25 mL) at 0° C. under nitrogen was dropwise added methanesulfonyl chloride (3.2086 g, 28.010 mmol) and the reaction mixture was stirred for 1 hour. After completion, the reaction was quenched cold with saturated aqueous sodium bicarbonate (20 mL). Two layers were separated, and the aqueous layer was extracted with DCM (2×25 mL). Combined organic layers were washed with brine (20 mL), dried over anhydrous sodium sulfate and concentrated to afford [3-methyl-1-(5-oxotetrahydrofuran-2-yl)butyl]methanesulfonate, diastereomer 2 (5.98 g, 96%) as yellow oil. ¹H NMR (250 MHz, CDCl₃) δ 5.06-4.91 (m, 1H), 4.60 (td, J=7.5, 2.9 Hz, 1H), 3.05 (s, 3H), 2.68-2.48 (m, 2H), 2.36-2.17 (m, 2H), 1.90-1.54 (m, 2H), 1.45-1.25 (m, 1H), 1.06-0.87 (m, 6H).

### Step 4: 5-(1-Azido-3-methyl-butyl)tetrahydrofuran-2-one, diastereomer 2

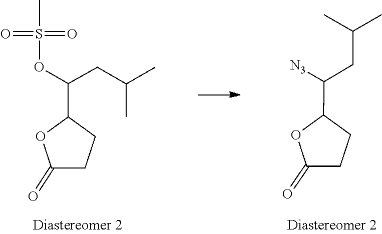 [SMILES: CC(C)CC(OS(C)(=O)=O)C1CCC(=O)O1 ; CC(C)CC(N=[N+]=[N-])C1CCC(=O)O1]

To a stirring solution of [3-methyl-1-(5-oxotetrahydrofuran-2-yl)butyl]methanesulfonate, diastereomer 2 (5.98 g, 23.890 mmol) in anhydrous DMF (25 mL) under nitrogen was added sodium azide (1.8637 g, 28.668 mmol) in one portion and the reaction mixture was heated to 80° C. for 8 hours. After completion, the reaction mixture was cooled to room temperature and diluted with water (400 mL). The product was extracted with ethyl acetate (3×150 mL). Combined organic layers were washed with water (200 mL), brine (100 mL), dried over anhydrous sodium sulfate and concentrated to afford 5-(1-azido-3-methyl-butyl)tetrahydrofuran-2-one, diastereomer 2 (4.33 g, 87%) as yellow oil. The product was carried to the next step without further purification. ¹H NMR (250 MHz, CDCl₃) δ 4.50 (m, 1H), 3.46-3.29 (m, 1H), 2.75-2.43 (m, 2H), 2.41-2.21 (m, 1H), 2.20-2.00 (m, 1H), 1.96-1.74 (m, 1H), 1.73-1.55 (m, 1H), 1.48-1.28 (m, 1H), 1.08-0.84 (m, 6H).

### Step 5: 5-Hydroxy-6-isobutyl-piperidin-2-one, diastereomer 2

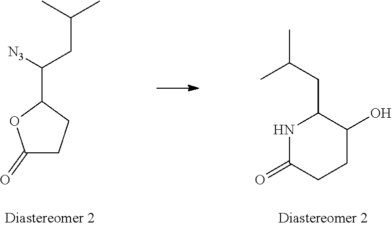

To a stirring solution of 5-(1-azido-3-methyl-butyl)tetrahydrofuran-2-one, diastereomer 2 (4.33 g, 21.954 mmol) in anhydrous methanol (80 mL) was added palladium hydroxide (1 g, 20% w/w, 1.4241 mmol). The reaction mixture was stirred under hydrogen (1 atm) at room temperature for 48 hours. The reaction mixture was filtered through a pad of Celite and concentrated under vacuum to afford 5-hydroxy-6-isobutyl-piperidin-2-one, diastereomer 2 (3.9 g, 93%) as yellow oil. The product was used in the next step without further purification. ¹H NMR (250 MHz, CDCl₃) δ 5.83 (s, 1H), 4.11-3.84 (m, 1H), 3.55-3.31 (m, 1H), 3.01-2.40 (m, 2H), 2.40-2.18 (m, 1H), 2.18-1.97 (m, 1H), 1.97-1.59 (m, 2H), 1.59-1.26 (m, 2H), 1.04-0.73 (m, 6H). ESI-MS m/z calc. 171.12593, found 172.6 (M+1)⁺; Retention time: 1.89 minutes; LC method S.

### Step 6: 2-Isobutylpiperidin-3-ol, diastereomer 2

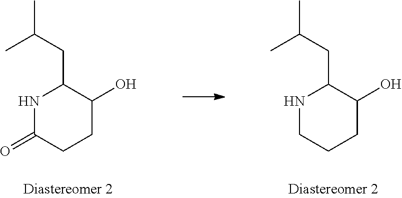

To a stirring solution of 5-hydroxy-6-isobutyl-piperidin-2-one, diastereomer 2 (3.9 g, 22.775 mmol) in anhydrous THE (150 mL) at room temperature under nitrogen was dropwise added boron dimethyl sulfide (34.163 mL of 2 M, 68.325 mmol). After the addition was complete, the reaction mixture was heated to 70° C. for 2 hours. The reaction mixture was cooled to 0° C. and slowly quenched with water (75 mL). Volatiles were removed under vacuum and the aqueous layer was basified to pH ˜11 with 1 M aqueous sodium hydroxide. The product was extracted with chloroform (3×120 mL), combined organic layers were washed with brine (30 mL), dried over anhydrous sodium sulfate and concentrated to afford 2-isobutylpiperidin-3-ol, diastereomer 2 (3.52 g, 88%) as white wax. The product was taken to the next step without further purification. ¹H NMR (250 MHz, CDCl₃) δ 3.87-3.44 (m, 1H), 2.87-2.36 (m, 2H), 2.27-1.06 (m, 10H), 1.05-0.71 (m, 6H). ESI-MS m/z calc. 157.14667, found 158.4 (M+1)⁺; Retention time: 1.24 minutes; LC method S.

### Step 7: 3-[[4-Chloro-6-(2,6-dimethylphenyl)pyrimidin-2-yl]sulfamoyl]benzoyl chloride

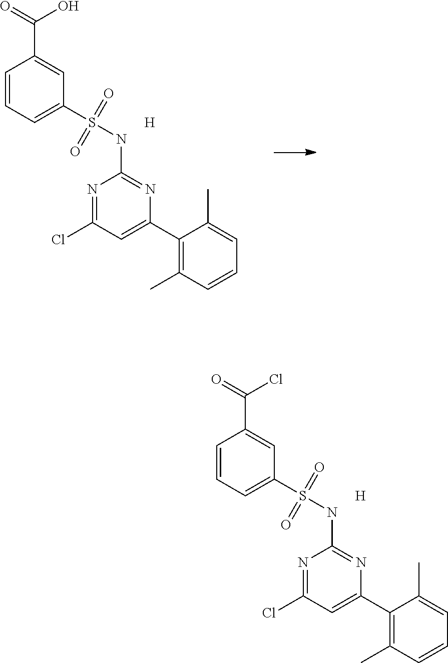

To a stirring suspension of 3-[[4-chloro-6-(2,6-dimethylphenyl)pyrimidin-2-yl]sulfamoyl]benzoic acid (420 mg, 1.0051 mmol) in thionyl chloride (8.1550 g, 5 mL, 68.546 mmol) at room temperature was added DMF (9.4400 mg, 10 μL, 0.1291 mmol) and the resulting mixture was heated to 45° C. for 18 hours. After completion, the volatiles were removed under vacuum to afford crude 3-[[4-chloro-6-(2,6-dimethylphenyl)pyrimidin-2-yl]sulfamoyl]benzoyl chloride (478 mg, 32%) as yellow solid that was taken to the next step without further purification. ESI-MS m/z calc. 435.02112, found 436.4 (M+1)⁺; Retention time: 6.56 minutes; (LC method S).

### Step 8: N-[4-Chloro-6-(2,6-dimethylphenyl)pyrimidin-2-yl]-3-(3-hydroxy-2-isobutyl-piperidine-1-carbonyl)benzenesulfonamide, diastereomer 2

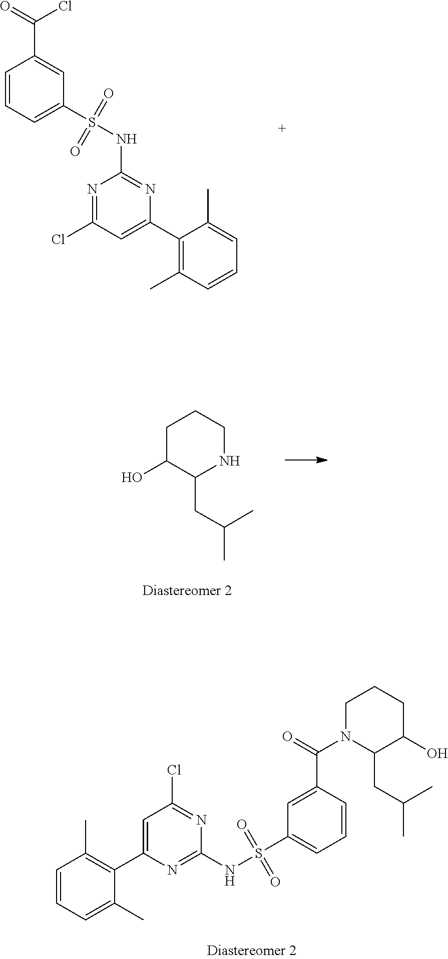 [SMILES: Cc1cccc(C)c1-c1cc(Cl)nc(NS(=O)(=O)c2cccc(C(=O)Cl)c2)n1 ; CC(C)CC1NCCCC1O ; Cc1cccc(C)c1-c1cc(Cl)nc(NS(=O)(=O)c2cccc(C(=O)N3CCCC(O)C3CC(C)C)c2)n1]

To a stirring solution of crude 3-[[4-chloro-6-(2,6-dimethylphenyl)pyrimidin-2-yl]sulfamoyl]benzoyl chloride (478 mg, 0.3177 mmol) and TEA (726.00 mg, 1 mL, 7.1746 mmol) in DCM (5 mL) at 0° C. was dropwise added a solution of 2-isobutylpiperidin-3-ol, diastereomer 2 (110 mg, 0.6995 mmol) in DCM (5 mL). After the addition was complete, the reaction mixture was allowed to warm up to room temperature and stirred for 1 hour. After completion, the reaction was quenched with 1 M aqueous hydrochloric acid (10 mL). Two layers were separated, and the aqueous layer was extracted with DCM (2×25 mL). Combined organic layers were washed with brine (20 mL), dried over anhydrous sodium sulfate and concentrated. The product was purified by silica gel chromatography using 0-65% hexanes-ethyl acetate to afford N-[4-chloro-6-(2,6-dimethylphenyl)pyrimidin-2-yl]-3-(3-hydroxy-2-isobutyl-piperidine-1-carbonyl)benzenesulfonamide, diastereomer 2 (120 mg, 58%) as a white solid. ESI-MS m/z calc. 556.1911, found 557.6 (M+1)⁺; Retention time: 5.71 minutes; LC method S.

### Step 9: 18-(2,6-Dimethylphenyl)-22-(2-methylpropyl)-2-oxa-14λ⁶-thia-7,15,17,20-tetraazatetracyclo[14.3.1.13,7.19,13]docosa-1(20),9(21),10,12,16,18-hexaene-8,14,14-trione (Compound 230)

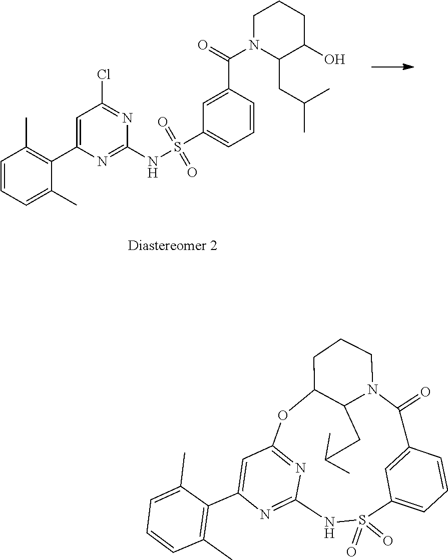 [SMILES: Cc1cccc(C)c1-c1cc(Cl)nc(NS(=O)(=O)c2cccc(C(=O)N3CCCC(O)C3CC(C)C)c2)n1 ; Cc1cccc(C)c1-c1cc2nc(n1)NS(=O)(=O)c1cccc(c1)C(=O)N1CCCC(O2)C1CC(C)C]

To a stirring solution of N-[4-chloro-6-(2,6-dimethylphenyl)pyrimidin-2-yl]-3-(3-hydroxy-2-isobutyl-piperidine-1-carbonyl)benzenesulfonamide diastereomer 2 (120 mg, 0.1831 mmol) in anhydrous DMF (10 mL) at room temperature under nitrogen was added sodium hydride (100 mg, 60% w/w, 2.5002 mmol) and the reaction mixture was stirred for 24 hours. After completion, the reaction mixture was cooled to 0° C. and quenched with 10% aqueous citric acid (10 mL) and water (50 mL). The product was extracted with ethyl acetate (3×50 mL). Combined organic layers were washed with brine (2×40 mL), dried over anhydrous sodium sulfate and concentrated. The product was purified by reverse phase HPLC using water (5 mM HCl buffer)-acetonitrile gradient method (C18 Higgins Analytical column, 20-80% acetonitrile, 40 mL/min) to afford 18-(2,6-dimethylphenyl)-22-(2-methylpropyl)-2-oxa-14λ⁶-thia-7,15,17,20-tetraazatetracyclo[14.3.1.13,7.19,13]docosa-1(20),9(21),10,12,16,18-hexaene-8,14,14-trione (23 mg, 24%) as white solid. ¹H NMR (250 MHz, DMSO(d6)) δ 8.46 (s, 1H), 7.93 (s, 1H), 7.67 (s, 2H), 7.27 (t, J=7.5 Hz, 1H), 7.13 (d, J=7.6 Hz, 2H), 6.41 (s, 1H), 5.32 (s, 1H), 4.32 (d, J=13.5 Hz, 1H), 4.16 (s, 1H), 2.90 (d, J=10.8 Hz, 1H), 2.23-1.64 (m, 11H), 1.12 (s, 1H), 0.97-0.78 (m, 1H), 0.69 (d, J=6.5 Hz, 3H), 0.02 (d, J=6.3 Hz, 3H). ESI-MS m/z calc. 520.2144, found 521.5 (M+1)⁺; Retention time: 2.44 minutes; LC method T.

### Example 128: Preparation of Compound 231

### Step 1: 18-(2,6-Dimethylphenyl)-22-(2-methylpropyl)-2-oxa-14λ⁶-thia-7,15,17,20-tetraazatetracyclo[14.3.1.13,7.19,13]docosa-1(20),9(21),10,12,16,18-hexaene-8,14,14-trione (Compound 231)

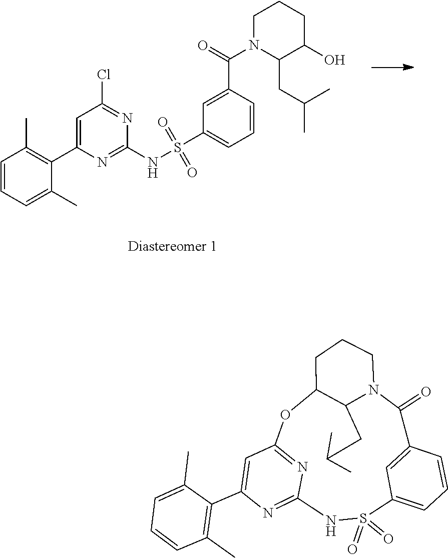 [SMILES: Cc1cccc(C)c1-c1cc(Cl)nc(NS(=O)(=O)c2cccc(C(=O)N3CCCC(O)C3CC(C)C)c2)n1 ; Cc1cccc(C)c1-c1cc2nc(n1)NS(=O)(=O)c1cccc(c1)C(=O)N1CCCC(O2)C1CC(C)C]

To a stirring solution of N-[4-chloro-6-(2,6-dimethylphenyl)pyrimidin-2-yl]-3-(3-hydroxy-2-isobutyl-piperidine-1-carbonyl)benzenesulfonamide (diastereomer 1, prepared in a manner analogous to that described above for diastereomer 2, 480 mg, 0.8616 mmol) in anhydrous DMF (50 mL) at room temperature under nitrogen was added sodium hydride (344.61 mg, 60% w/w, 8.6160 mmol) in several portions and the reaction mixture was stirred for 40 hours. After completion, the reaction mixture was cooled to 0° C. and quenched with 10% aqueous citric acid (40 mL) and water (100 mL). The product was extracted with ethyl acetate (3×100 mL). Combined organic layers were washed with brine (2×75 mL), dried over anhydrous sodium sulfate and concentrated. The crude was purified by silica gel chromatography using 0-65% hexanes-ethyl acetate, followed by reverse phase HPLC using water (5 mM HCl buffer)-acetonitrile gradient method (Cis Higgins Analytical column, 30-60% acetonitrile, 25 mL/min) to afford 18-(2,6-dimethylphenyl)-22-(2-methylpropyl)-2-oxa-14λ⁶-thia-7,15,17,20-tetraazatetracyclo[14.3.1.13,7.19,13]docosa-1(20),9(21),10,12,16,18-hexaene-8,14,14-trione (76 mg, 17%) as white solid. ¹H NMR (250 MHz, DMSO(d6)) δ 8.46 (s, 1H), 7.94-7.75 (m, 1H), 7.59 (d, J=5.5 Hz, 2H), 7.29-7.16 (m, 1H), 7.10 (d, J=7.6 Hz, 2H), 6.99 (s, 1H), 4.61 (s, 1H), 4.48 (d, J=13.2 Hz, 1H), 3.05 (dd, J=9.3, 4.4 Hz, 1H), 2.97-2.78 (m, 1H), 2.06 (d, J=6.2 Hz, 2H), 1.91 (s, 7H), 1.65 (dd, J=17.5, 9.0 Hz, 2H), 1.43-1.08 (m, 2H), 0.62 (d, J=5.6 Hz, 3H), 0.30 (d, J=5.5 Hz, 3H). ESI-MS m/z calc. 520.2144, found 521.5 (M+1)⁺; Retention time: 2.41 minutes; LC method T.

### Example 129: Preparation of Compound 232

### Step 1: 4-Hydroxy-2,2-dimethyl-butanoic acid

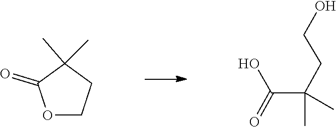

To a solution of 3,3-dimethyltetrahydrofuran-2-one (7.87 g, 68.949 mmol) in mixture of MeOH (34 mL) and water (34 mL) was added NaOH (3.17 g, 79.256 mmol). The resulting solution was heated at 45° C. for 16 hours. All solvents were removed under reduced pressure. The obtained solid was treated with toluene (50 mL) and the toluene was removed under reduced pressure. Then, the obtained solid was dissolved in water (60 mL) and cooled to 0° C. Aqueous hydrochloric acid (6N) was added slowly until pH value reached 4. Aqueous solution was extracted with 2-methyl THE (6×80 mL). Combined organic layer was washed with brine (30 mL) and dried over sodium sulfate, filtered and concentrated under the reduced pressure to 4-hydroxy-2,2-dimethyl-butanoic acid (6.11 g, 63%) as a pale-yellow oil. ¹H NMR (250 MHz, CDCl₃) δ 3.74 (t, J=6.6 Hz, 1H), 1.86 (t, J=6.6 Hz, 1H), 1.25 (s, 6H).

### Step 2: 5-Hydroxy-3,3-dimethyl-tetrahydrofuran-2-one

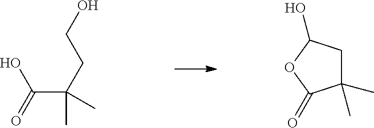

To a solution of 4-hydroxy-2,2-dimethyl-butanoic acid (6.11 g, 43.459 mmol) in anhydrous DCM (435 mL) at ambient temperature was added Dess-Martin periodinane (20.001 g, 45.741 mmol). The resulting solution was stirred at ambient temperature for 100 minutes, filtered and washed with DCM (100 mL). Filtrate was concentrated under the reduced pressure. The residue obtained was purified by flash chromatography (loaded in DCM) (220 g silica gel, eluting 0 to 40% EtOAc/hexanes), product fractions were combined and concentrated in vacuo to afford 5-hydroxy-3,3-dimethyl-tetrahydrofuran-2-one (3.318 g, 59%) as a white solid. ¹H NMR (250 MHz, CDCl₃) δ 5.87 (t, J=4.6 Hz, 1H), 4.44 (s, 1H), 2.38-2.23 (m, 1H), 2.12-1.98 (m, 1H), 1.38 (s, 3H), 1.27 (s, 3H).

### Step 3: (E)-2,2,7-Trimethyloct-4-enoic acid

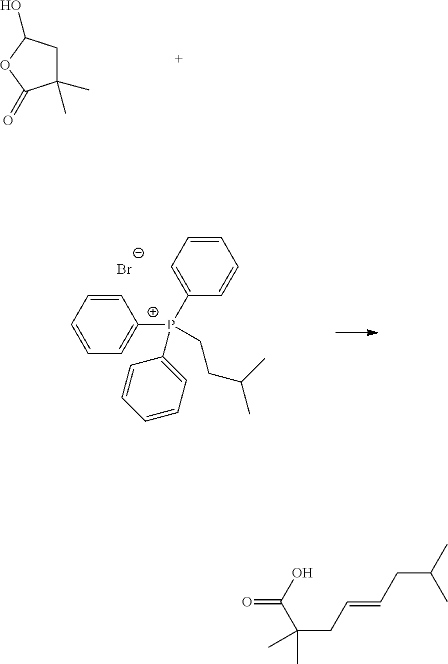

To a stirring suspension of isoamyltriphenylphosphonium bromide (42.060 g, 101.76 mmol) in anhydrous THE (120 mL) at −78° C. under nitrogen was dropwise added n-BuLi (40.704 mL of 2.5 M in hexanes, 101.76 mmol). After the addition was complete, the reaction mixture was warmed up to 0° C. and stirred for 1 hour. After cooling to −78° C., a solution of 5-hydroxy-3,3-dimethyl-tetrahydrofuran-2-one (3.311 g, 25.441 mmol) in anhydrous THE (60 mL) was added dropwise. After the addition was complete, the reaction mixture was allowed to warm up to room temperature and stirred for 48 hours. The reaction was quenched with water (80 mL) and 1 M aqueous hydrochloric acid (20 mL). Volatiles were removed under vacuum and the aqueous layer was extracted with ethyl acetate (3×120 mL). Combined organic layers were washed with brine (30 mL), dried over anhydrous sodium sulfate and concentrated. The crude was purified by silica gel chromatography using 0-25% hexanes-ethyl acetate to afford (E)-2,2,7-trimethyloct-4-enoic acid (3.94 g, 80%) as a pale-yellow oil. ¹H NMR (250 MHz, CDCl₃) δ 5.63-5.27 (m, 2H), 2.38-2.18 (m, 2H), 2.00-1.82 (m, 2H), 1.72-1.48 (m, 1H), 1.19 (s, 6H), 0.87 (d, J=6.5 Hz, 6H).

### Step 4: 5-(1-Hydroxy-3-methyl-butyl)-3,3-dimethyl-tetrahydrofuran-2-one diastereomer 1 and 2

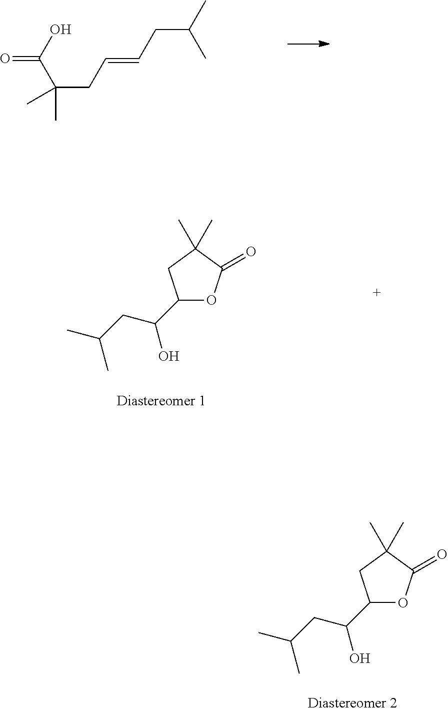

To a stirring suspension of methylrhenium(VII) trioxide (303.75 mg, 1.2187 mmol) in chloroform (25 mL) at room temperature was added hydrogen peroxide (1.9346 g, 1.7429 mL of 50% w/w, 28.437 mmol), followed by the addition of a solution of (E)-2,2,7-trimethyloct-4-enoic acid (3.94 g, 20.312 mmol) in acetonitrile (25 mL). The reaction mixture was stirred for 18 hours. The reaction was quenched with 1 M aqueous Na₂CO₃ (20 mL) and water (40 mL). Volatiles were removed under vacuum and the product was extracted with ethyl acetate (3×100 mL). Combined organic layers were washed with brine (40 mL), dried over anhydrous sodium sulfate and concentrated. The product was purified by silica gel chromatography using 0-30% hexanes-diethyl ether to afford 5-(1-hydroxy-3-methyl-butyl)-3,3-dimethyl-tetrahydrofuran-2-one (diastereomer 1, 3.05 g, 72%) as colorless oil and 5-(1-hydroxy-3-methyl-butyl)-3,3-dimethyl-tetrahydrofuran-2-one (diastereomer 2, 760 mg, 18%) as colorless oil. Diastereomer 1: ¹H NMR (250 MHz, CDCl₃) δ 4.37-4.18 (m, 1H), 3.70-3.50 (m, 1H), 2.09-1.80 (m, 4H), 1.59-1.43 (m, 1H), 1.28 (d, J=3.0 Hz, 6H), 1.23-1.10 (m, 1H), 0.94 (dd, 6H). Diastereomer 2: ¹H NMR (250 MHz, CDCl₃) δ 4.43-4.26 (m, 1H), 4.15-3.97 (m, 1H), 2.24-2.08 (m, 1H), 2.01-1.72 (m, 3H), 1.45-1.34 (m, 1H), 1.28 (d, 6H), 1.22-1.05 (m, 1H), 0.95 (dd, 6H).

### Step 5: [1-(4,4-Dimethyl-5-oxo-tetrahydrofuran-2-yl)-3-methyl-butyl]methanesulfonate, diastereomer 1

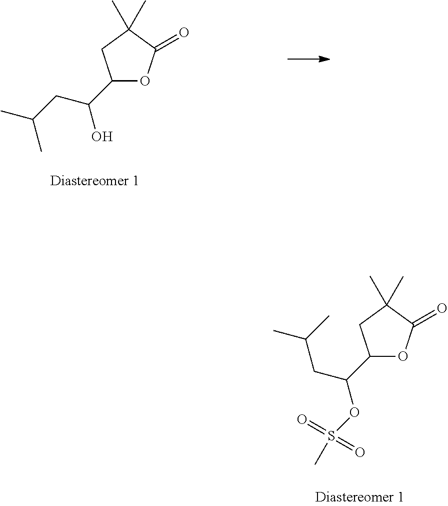 [SMILES: CC(C)CC(O)C1CC(C)(C)C(=O)O1 ; CC(C)CC(OS(C)(=O)=O)C1CC(C)(C)C(=O)O1]

To a stirring solution of 5-(1-hydroxy-3-methyl-butyl)-3,3-dimethyl-tetrahydrofuran-2-one, diastereomer 1 (3.05 g, 15.229 mmol) and TEA (2.3115 g, 3.1839 mL, 22.843 mmol) in anhydrous DCM (20 mL) at 0° C. under nitrogen was added dropwise methanesulfonyl chloride (2.0934 g, 1.4145 mL, 18.275 mmol). After the addition was complete, the reaction mixture was stirred for 1 hour. The reaction was quenched saturated aqueous sodium bicarbonate (20 mL), and two layers were separated. The aqueous layers were extracted with DCM (2×20 mL). Combined organic layers were washed with brine (15 mL), dried over anhydrous sodium sulfate and concentrated to afford [1-(4,4-dimethyl-5-oxo-tetrahydrofuran-2-yl)-3-methyl-butyl]methanesulfonate diastereomer 1 (4.196 g, 93%) as yellow oil. The product was carried to the next step without further purification. ¹H NMR (250 MHz, CDCl₃) δ 4.79-4.65 (m, 1H), 4.55-4.38 (m, 1H), 3.15 (s, 3H), 2.19-2.04 (m, 1H), 1.97-1.76 (m, 2H), 1.75-1.56 (m, 2H), 1.28 (d, 6H), 0.97 (dd, J=6.6, 3.5 Hz, 6H).

### Step 6: 5-(1-Azido-3-methyl-butyl)-3,3-dimethyl-tetrahydrofuran-2-one diastereomer 1

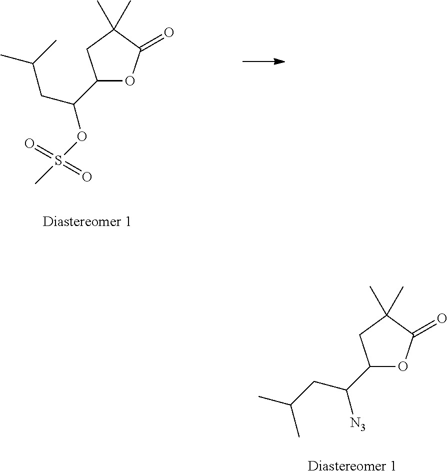 [SMILES: CC(C)CC(OS(C)(=O)=O)C1CC(C)(C)C(=O)O1 ; CC(C)CC(N=[N+]=[N-])C1CC(C)(C)C(=O)O1]

To a stirring solution of [1-(4,4-dimethyl-5-oxo-tetrahydrofuran-2-yl)-3-methyl-butyl]methanesulfonate, diastereomer 1 (4.19 g, 15.052 mmol) in anhydrous DMF (20 mL) under nitrogen was added sodium azide (1.1742 g, 3.5336 mL, 18.062 mmol) and the reaction mixture was heated to 80° C. for 16 hours. After cooling to room temperature, the reaction mixture was diluted with water (50 mL) and brine (30 mL) and the product was extracted with ethyl acetate (3×120 mL). Combined organic layers were washed with brine (2×60 mL), dried over anhydrous sodium sulfate and concentrated to afford 5-(1-azido-3-methyl-butyl)-3,3-dimethyl-tetrahydrofuran-2-one, diastereomer 1 (3.05 g, 85%) as an amber oil. The product was carried to the next step without further purification. ¹H NMR (250 MHz, CDCl₃) δ 4.49-4.24 (m, 1H), 3.82-3.62 (m, 1H), 2.14-1.93 (m, 2H), 1.92-1.70 (m, 1H), 1.46-1.17 (m, 8H), 1.06-0.84 (m, 6H).

### Step 7: 5-Hydroxy-6-isobutyl-3,3-dimethyl-piperidin-2-one, diastereomer 1

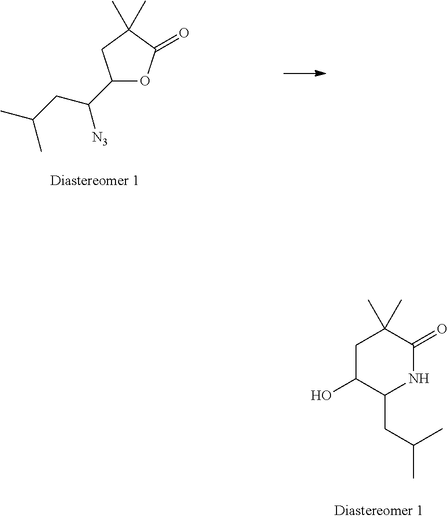 [SMILES: CC(C)CC(N=[N+]=[N-])C1CC(C)(C)C(=O)O1 ; CC(C)CC1NC(=O)C(C)(C)CC1O]

To a stirring solution of 5-(1-azido-3-methyl-butyl)-3,3-dimethyl-tetrahydrofuran-2-one, diastereomer 1 (3.05 g, 13.538 mmol) in anhydrous methanol (50 mL) was added palladium hydroxide (570.38 mg, 20% w/w, 0.8123 mmol) and the reaction mixture was kept under hydrogen (1 atm) at room temperature for 24 hours. The reaction mixture was filtered through a pad of Celite and concentrated. The crude was purified by silica gel chromatography using 0-40% hexanes-ethyl acetate to afford 5-hydroxy-6-isobutyl-3,3-dimethyl-piperidin-2-one, diastereomer 1 (2.21 g, 73%) as pale-yellow oil. ¹H NMR (250 MHz, CDCl₃) δ 4.59-4.18 (m, 1H), 3.31-3.06 (m, 1H), 2.20-1.54 (m, 5H), 1.47-1.09 (m, 8H), 1.06-0.75 (m, 6H). ESI-MS m/z calc. 199.15723, found 200.7 (M+1)⁺; Retention time: 2.41 minutes; LC method S.

### Step 8: 2-Isobutyl-5,5-dimethyl-piperidin-3-ol, diastereomer 1

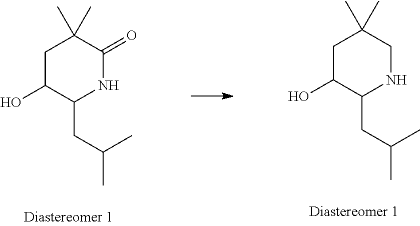

To a stirring solution of 5-hydroxy-6-isobutyl-3,3-dimethyl-piperidin-2-one, diastereomer 1 (325 mg, 1.6308 mmol) in anhydrous THE (10 mL) at room temperature under nitrogen was dropwise added boron dimethyl sulfide (2.4462 mL of 2 M, 4.8924 mmol). After the addition was complete, the reaction mixture was heated to 70° C. for 2 hours. After cooling to 0° C., the reaction was quenched with water (10 mL). Volatiles were removed under vacuum and the aqueous layer was basified with 1 M aqueous sodium hydroxide to pH 11. The product was extracted with chloroform (3×30 mL). Combined organic layers were washed with brine, dried over anhydrous sodium sulfate and concentrated. The crude was purified by reverse phase HPLC using water (0.1% TFA buffer)-acetonitrile (0.1% TFA buffer) gradient method (C₁₈ Varian column, 5-55% acetonitrile, 40 mL/min) to afford 2-isobutyl-5,5-dimethyl-piperidin-3-ol, diastereomer 1 (18 mg, 6%) as white solid. ESI-MS m/z calc. 185.17796, found 186.4 (M+1)⁺; Retention time: 0.86 minutes; LC method T.

### Step 9: N-[4-Chloro-6-(2,6-dimethylphenyl)pyrimidin-2-yl]-3-(3-hydroxy-2-isobutyl-5,5-dimethyl-piperidine-1-carbonyl)benzenesulfonamide, diastereomer 1

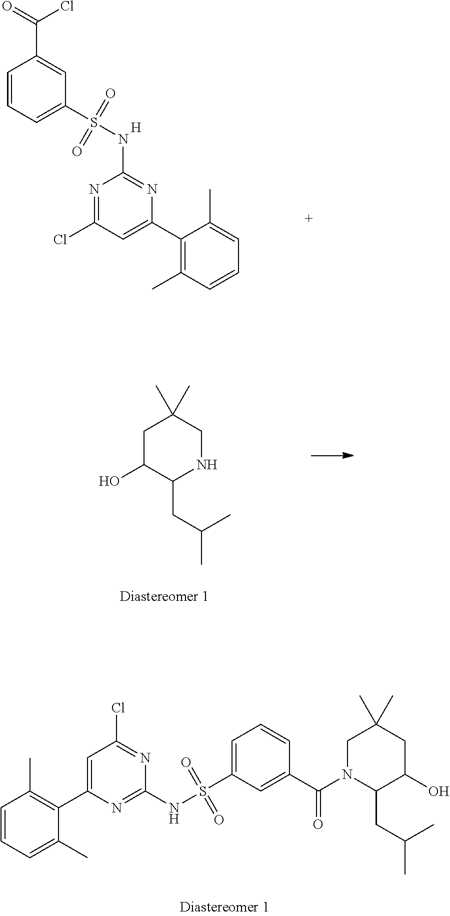

To a stirring solution of 2-isobutyl-5,5-dimethyl-piperidin-3-ol, diastereomer 1(18 mg, 0.0971 mmol) and TEA (145.20 mg, 200 μL, 1.4349 mmol) in DCM (2 mL) at 0° C. was dropwise added a solution of 3-[[4-chloro-6-(2,6-dimethylphenyl)pyrimidin-2-yl]sulfamoyl]benzoyl chloride (55 mg, 0.1008 mmol) in DCM (2 mL). After the addition was complete, the reaction mixture was stirred for 1 hour. The reaction was quenched with 1 M aqueous hydrochloric acid (2 mL) and water (10 mL). 5 mL of DCM was added, and two layers were separated. The aqueous layer was extracted with DCM (2×20 mL). Combined organic layers were washed with brine (10 mL), dried over anhydrous sodium sulfate and concentrated. The crude was purified by silica gel chromatography using 0-40% hexanes-acetone to afford N-[4-chloro-6-(2,6-dimethylphenyl)pyrimidin-2-yl]-3-(3-hydroxy-2-isobutyl-5,5-dimethyl-piperidine-1-carbonyl)benzenesulfonamide (48 mg, 80%) as white solid. ESI-MS m/z calc. 584.2224, found 585.6 (M+1)⁺; Retention time: 6.3 minutes; LC method S.

### Step 10: 18-(2,6-Dimethylphenyl)-5,5-dimethyl-22-(2-methylpropyl)-2-oxa-14λ⁶-thia-7,15,17,20-tetraazatetracyclo[14.3.1.13,7.19,13]docosa-1(20),9(21),10,12,16,18-hexaene-8,14,14-trione (Compound 232)

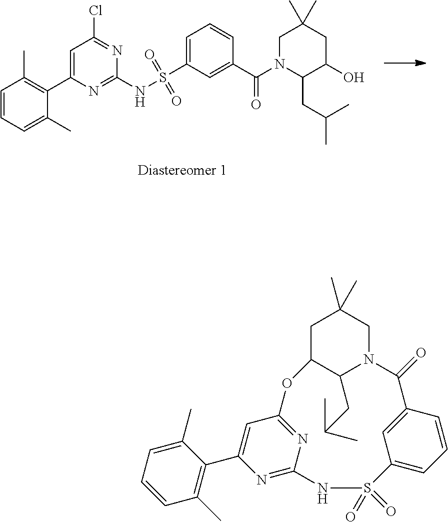 [SMILES: Cc1cccc(C)c1-c1cc(Cl)nc(NS(=O)(=O)c2cccc(C(=O)N3CC(C)(C)CC(O)C3CC(C)C)c2)n1 ; Cc1cccc(C)c1-c1cc2nc(n1)NS(=O)(=O)c1cccc(c1)C(=O)N1CC(C)(C)CC(O2)C1CC(C)C]

To a stirring solution of N-[4-chloro-6-(2,6-dimethylphenyl)pyrimidin-2-yl]-3-(3-hydroxy-2-isobutyl-5,5-dimethyl-piperidine-1-carbonyl)benzenesulfonamide, diastereomer 1 (48 mg, 0.0820 mmol) in anhydrous DMF (5 mL) at room temperature under nitrogen was added sodium hydride (32.797 mg, 60% w/w, 0.8200 mmol) in one portion. The reaction mixture was stirred for 16 hours. After cooling to 0° C., the reaction was quenched with 10% aqueous citric acid (5 mL). Water was added (60 mL) and the product was extracted with ethyl acetate (3×40 mL). Combined organic layers were washed with brine (2×20 mL), dried over anhydrous sodium sulfate and concentrated. The crude was purified by silica gel chromatography using 0-40% hexanes-acetone to afford 18-(2,6-Dimethylphenyl)-5,5-dimethyl-22-(2-methylpropyl)-2-oxa-14λ⁶-thia-7,15,17,20-tetraazatetracyclo[14.3.1.13,7.19,13]docosa-1(20),9(21),10,12,16,18-hexaene-8,14,14-trione (11 mg, 23%) as white solid. ¹H NMR (250 MHz, DMSO-d₆) δ 8.45 (s, 1H), 7.85 (s, 1H), 7.60 (s, 2H), 7.28-7.16 (m, 1H), 7.15-7.04 (m, 2H), 6.96 (s, 1H), 4.63 (s, 1H), 4.22 (d, J=13.1 Hz, 1H), 3.13-2.97 (m, 1H), 2.69 (d, J=13.3 Hz, 1H), 2.01-1.77 (m, 7H), 1.53-1.49 (m, 1H), 1.29-1.18 (m, 6H), 1.03 (s, 3H), 0.58 (d, J=5.2 Hz, 3H), 0.26 (d, J=5.2 Hz, 3H). ESI-MS m/z calc. 548.2457, found 549.4 (M+1)⁺; Retention time: 2.68 minutes; LC method S.

### Example 130: Preparation of Compound 233

### Step 1: 18-(2,6-dimethylphenyl)-5,5-dimethyl-22-(2-methylpropyl)-2-oxa-14λ⁶-thia-7,15,17,20-tetraazatetracyclo[14.3.1.13,7.19,13]docosa-1(20),9(21),10,12,16,18-hexaene-8,14,14-trione (Compound 233)

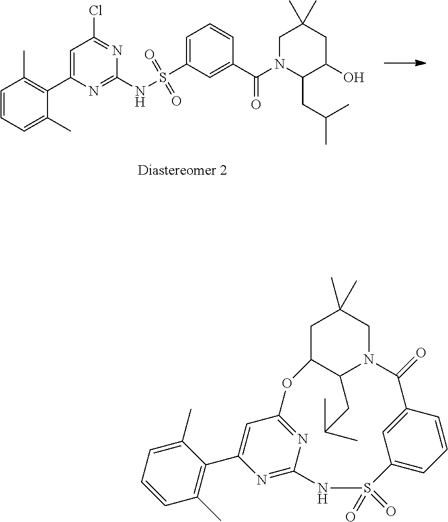 [SMILES: Cc1cccc(C)c1-c1cc(Cl)nc(NS(=O)(=O)c2cccc(C(=O)N3CC(C)(C)CC(O)C3CC(C)C)c2)n1 ; Cc1cccc(C)c1-c1cc2nc(n1)NS(=O)(=O)c1cccc(c1)C(=O)N1CC(C)(C)CC(O2)C1CC(C)C]

To a stirring solution of N-[4-chloro-6-(2,6-dimethylphenyl)pyrimidin-2-yl]-3-(3-hydroxy-2-isobutyl-5,5-dimethyl-piperidine-1-carbonyl)benzenesulfonamide, diastereomer 2, prepared in a manner analogous to that described above for diastereomer 1 (104 mg, 0.1777 mmol) in anhydrous DMF (10 mL) at room temperature under nitrogen was added sodium hydride (71.073 mg, 60% w/w, 1.7770 mmol) in one portion. The reaction mixture was stirred for 8 hours. After cooling to 0° C., the reaction was quenched with 10% aqueous citric acid (20 mL). Water was added (50 mL) and the product was extracted with ethyl acetate (3×50 mL). The combined organic layers were washed with brine (2×25 mL), dried over anhydrous sodium sulfate and concentrated. The crude was purified by silica gel chromatography using 0-35% hexanes-acetone to afford 18-(2,6-dimethylphenyl)-5,5-dimethyl-22-(2-methylpropyl)-2-oxa-14λ⁶-thia-7,15,17,20-tetraazatetracyclo[14.3.1.13,7.19,13]docosa-1(20),9(21),10,12,16,18-hexaene-8,14,14-trione (72 mg, 72%) as white solid. ESI-MS m/z calc. 548.2457, found 549.4 (M+1)⁺; Retention time: 2.76 minutes; LC method T. ¹H NMR (250 MHz, DMSO-d₆) δ 8.38 (s, 1H), 8.00-7.90 (m, 1H), 7.75-7.63 (m, 2H), 7.32-7.19 (m, 1H), 7.18-7.07 (m, 2H), 6.40 (s, 1H), 5.53-5.37 (m, 1H), 4.22-4.08 (m, 1H), 4.07-3.97 (m, 1H), 2.77 (d, J=13.6 Hz, 1H), 2.14-1.66 (m, 9H), 1.12 (m, 7H), 0.92-0.78 (m, 1H), 0.68 (d, J=6.5 Hz, 3H), 0.04 (d, J=6.5 Hz, 3H).

### Example 131: Preparation of Compound 234

### Step 1: [(2R)-5-Oxotetrahydrofuran-2-yl]methyl methanesulfonate

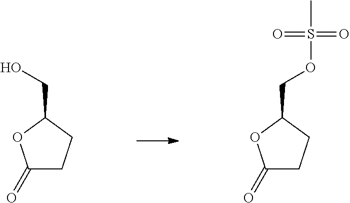

To a solution of (5R)-5-(hydroxymethyl)tetrahydrofuran-2-one (3 g, 25.836 mmol) in dichloromethane (75 mL) was added triethylamine (5.2272 g, 7.2 mL, 51.657 mmol) at room temperature and methanesulfonyl chloride (3.8480 g, 2.6 mL, 33.592 mmol) at room temperature and then the resulting mixture was stirred at that temperature for 1 hour. The resulting mixture was diluted with dichloromethane (25 mL) and quenched with a saturated aqueous solution of NaCl (50 mL). The aqueous layer was extracted two times with dichloromethane (2×50 mL). The combined organic layer was dried over sodium sulfate, filtered and concentrated under reduced pressure. Purification by silica gel column chromatography (40 g, heptanes/ethyl acetate=1:3) gave [(2R)-5-oxotetrahydrofuran-2-yl]methyl methanesulfonate (3.6 g, 72%) as a colorless oil; ¹H NMR (300 MHz, CDCl₃) δ 4.85-4.70 (m, 1H), 4.50-4.38 (m, 1H), 4.35-4.27 (m, 1H), 3.08 (s, 3H), 2.72-2.51 (m, 2H), 2.48-2.32 (m, 1H), 2.23-2.07 (m, 1H).

### Step 2: (5R)-5-(Azidomethyl)tetrahydrofuran-2-one

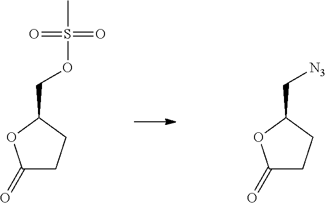

To a solution of [(2R)-5-oxotetrahydrofuran-2-yl]methyl methanesulfonate (3.6 g, 13.888 mmol) in dimethylformamide (69 mL) was added sodium azide (1.4 g, 21.535 mmol) at room temperature, and then the resulting mixture was stirred at 90° C. for 2 hours. The reaction was quenched with water (50 mL) at 0° C. The aqueous layer was extracted with diethyl ether (2×50 mL). The combined organic layer was dried over sodium sulfate, filtered and concentrated under reduced pressure. Purification by silica gel column chromatography (40 g, Heptane/EtOAc=1:3) gave (5R)-5-(azidomethyl)tetrahydrofuran-2-one (1.35 g, 69%) as clear oil; ¹H NMR (300 MHz, CDCl₃) δ 4.74-4.55 (m, 1H), 3.66-3.56 (m, 1H), 3.51-3.42 (m, 1H), 2.74-2.45 (m, 2H), 2.40-2.22 (m, 1H), 2.17-1.97 (m, 1H).

### Step 3: (5R)-5-(Azidomethyl)tetrahydrofuran-2-ol

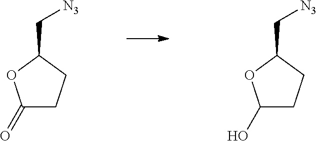 [SMILES: [N-]=[N+]=NC[C@H]1CCC(=O)O1 ; [N-]=[N+]=NC[C@H]1CCC(O)O1]

To a solution (−70° C.) of (5R)-5-(azidomethyl)tetrahydrofuran-2-one (1.8 g, 12.754 mmol) in tetrahydrofuran (5.4 mL) was added slowly a solution of diisobutylaluminum hydride (13 mL of 1 M, 13.000 mmol) in hexanes. The mixture was stirred for 45 minutes at −70° C. An additional portion of a solution of diisobutylaluminum hydride (3.9 mL of 1 M, 3.9000 mmol) in hexanes was added and the mixture was stirred at −60° C. for at least 6 hours. Then it was quenched by the addition of water (5 mL) at −60° C. with vigorous stirring. The mixture was allowed to reach room temperature and 0.5 M HCl (75 mL) and dichloromethane (200 mL) were added. The organic layer was separated, dried over sodium sulfate, filtered and evaporated. The residue was purified by column chromatography (40 g ethyl acetate) to provide (5R)-5-(azidomethyl)tetrahydrofuran-2-ol (1.02 g, 56%) as clear oil.

### Step 4: (3R)-6-Isobutylpiperidin-3-ol

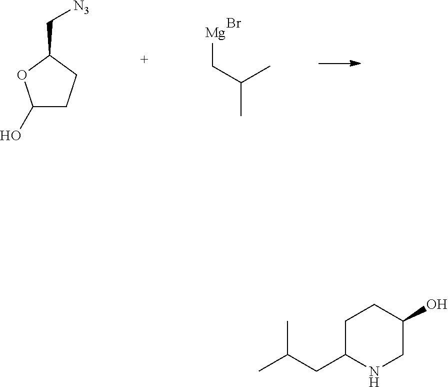

Trimethylphosphine (19 mL of 1 M, 19.000 mmol) in toluene was added to a solution of (5R)-5-(azidomethyl)tetrahydrofuran-2-ol (1.3 g, 9.0818 mmol) in methanol (26 mL) under nitrogen atmosphere. Upon consumption of the starting material (detected by TLC, eluent: heptane/EtOAc=1/1, v/v) and formation of the imine intermediate (detected by TLC, eluent: EtOAc/MeOH=10/1, v/v) the reaction mixture was concentrated under reduced pressure and the residue was co-evaporated twice with toluene (10 mL). The product was collected in a mixture of anhydrous tetrahydrofuran (26 mL) and toluene (26 mL) and then Isobutyl magnesium bromide (23 mL of 2 M, 46.000 mmol) in diethyl ether was added at 0° C. under nitrogen atmosphere. Upon consumption of the imine intermediate the reaction was quenched with NH₄Cl saturated (75 mL). The aqueous layer was extracted with ethyl acetate (3×50 mL), the combined organic phases were dried over sodium sulfate, filtered and concentrated in vacuo. The crude was dissolved in Methanolic HCl (5 mL of 3 M, 15.000 mmol) and then stirred for 2 hours and then concentrated under vacuum followed by purification by reverse phase chromatography (100 g, Initial 100% H₂O (0.1% formic Acid) to 50% CH₃CN (0.1% FA)) to give (3R)-6-isobutylpiperidin-3-ol (hydrochloride salt) (160 mg, 9%) as brownish solid; ESI-MS m/z calc. 157.14667, found 158.2 (M+1)⁺; Retention time: 0.74 minutes; LC method U.

### Step 5: (3R)-6-Isobutylpiperidin-3-ol

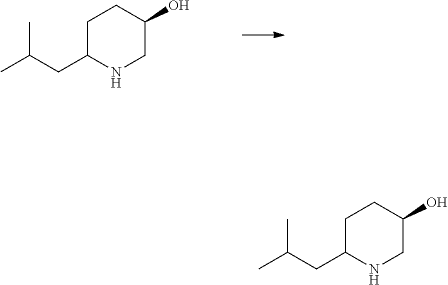

Combined two batches of (3R)-6-isobutylpiperidin-3-ol (hydrochloride salt) (212 mg, 1.0944 mmol) were dissolved in methanol (5 mL). The solvent was removed and concentrated under reduced pressure. The resulting brownish oil was dried under high vacuum overnight to provide (3R)-6-isobutylpiperidin-3-ol (hydrochloride salt) (190 mg, 87%) as a brownish oil. ¹H NMR (300 MHz, DMSO-d₆) δ 8.34 (br. s., 1H), 3.86 (br. s., 1H), 3.01-2.83 (m, 3H), 1.84-1.51 (m, 5H), 1.49-1.17 (m, 3H), 0.87 (d, J=6.8 Hz, 3H), 0.84 (d, J=6.5 Hz, 3H). ESI-MS m/z calc. 157.14667, found 158.2 (M+1)⁺; Retention time: 0.75 minutes; LC method U.

### Step 6: N-[4-Chloro-6-(2,6-dimethylphenyl)pyrimidin-2-yl]-3-[(5R)-5-hydroxy-2-isobutyl-piperidine-1-carbonyl]benzenesulfonamide

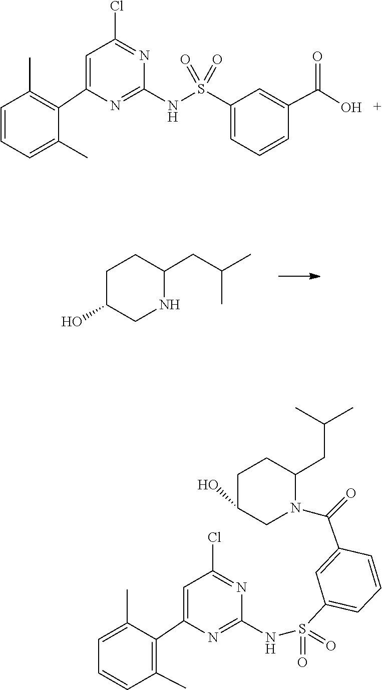 [SMILES: Cc1cccc(C)c1-c1cc(Cl)nc(NS(=O)(=O)c2cccc(C(=O)O)c2)n1 ; CC(C)CC1CC[C@@H](O)CN1 ; Cc1cccc(C)c1-c1cc(Cl)nc(NS(=O)(=O)c2cccc(C(=O)N3C[C@H](O)CCC3CC(C)C)c2)n1]

Into a solution of 3-[[4-chloro-6-(2,6-dimethylphenyl)pyrimidin-2-yl]sulfamoyl]benzoic acid (378 mg, 0.9046 mmol) in anhydrous DCM (2 mL) was added thionyl chloride (6.5240 g, 4 mL, 54.837 mmol). The reaction was stirred at 45° C. for 40 hours. LCMS indicated full conversion of the starting material. The volatiles were removed under vacuum. The residue was dissolved in anhydrous DCM (5 mL), which was added to a solution of (3R)-6-isobutylpiperidin-3-ol (hydrochloride salt) (190 mg, 0.9024 mmol) and triethylamine (290.40 mg, 0.4 mL, 2.8698 mmol) in anhydrous DCM (5 mL) at 0° C. dropwise. The reaction was stirred at room temperature for 2 hours. The reaction was quenched with 10% citric acid (15 mL) and extracted with ethyl acetate (3×15 mL). The combined organic layers were washed with brine (15 mL), dried over anhydrous sodium sulfate and concentrated under vacuum. The residue was purified by silica gel chromatography using 0 to 50% acetone in hexane to furnish N-[4-chloro-6-(2,6-dimethylphenyl)pyrimidin-2-yl]-3-[(5R)-5-hydroxy-2-isobutyl-piperidine-1-carbonyl]benzenesulfonamide (359 mg, 26%) as a clear oil. ESI-MS m/z calc. 556.1911, found 557.2 (M+1)⁺; Retention time: 6.0 minutes (LC method S).

### Step 7: (3R)-18-(2,6-Dimethylphenyl)-6-(2-methylpropyl)-2-oxa-14λ⁶-thia-7,15,17,20-tetraazatetracyclo[14.3.1.13,7.19,13]docosa-1(20),9(21),10,12,16,18-hexaene-8,14,14-trione (Compound 234)

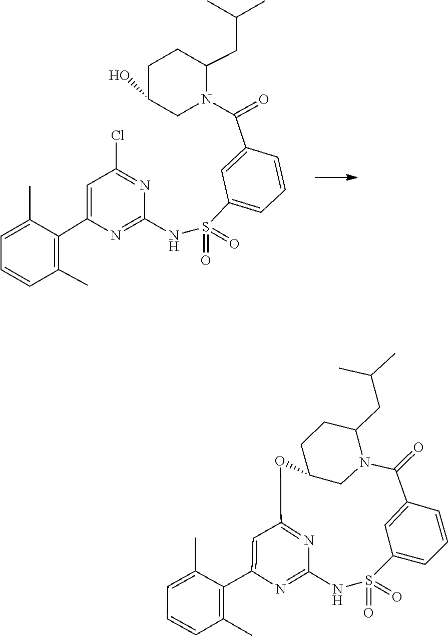 [SMILES: Cc1cccc(C)c1-c1cc(Cl)nc(NS(=O)(=O)c2cccc(C(=O)N3C[C@H](O)CCC3CC(C)C)c2)n1 ; Cc1cccc(C)c1-c1cc2nc(n1)NS(=O)(=O)c1cccc(c1)C(=O)N1C[C@@H](CCC1CC(C)C)O2]

Into a solution of N-[4-chloro-6-(2,6-dimethylphenyl)pyrimidin-2-yl]-3-[(5R)-5-hydroxy-2-isobutyl-piperidine-1-carbonyl]benzenesulfonamide (359 mg, 0.2320 mmol) in anhydrous DMF (20 mL) was added NaH (198 mg, 60% w/w, 4.9505 mmol). The reaction mixture was stirred at room temperature overnight. The reaction was quenched with 10% citric acid (30 mL). The aqueous solution was extracted with ethyl acetate (3×30 mL). The combined organic layers were washed with brine (30 mL), dried over anhydrous sodium sulfate and concentrated under vacuum. The residue was purified by silica gel chromatography using 0 to 50% acetone in hexane to furnish (3R)-18-(2,6-dimethylphenyl)-6-(2-methylpropyl)-2-oxa-14λ⁶-thia-7,15,17,20-tetraazatetracyclo[14.3.1.13,7.19,13]docosa-1(20),9(21),10,12,16,18-hexaene-8,14,14-trione (47.4 mg, 38%) as a white powder. ESI-MS m/z calc. 520.2144, found 521.2 (M+1)⁺; Retention time: 2.68 minutes; LC method W. ¹H NMR (500 MHz, DMSO-d₆) δ 8.48 (s, 1H), 7.91 (s, 1H), 7.64 (s, 2H), 7.26 (t, J=7.7 Hz, 1H), 7.13 (d, J=7.6 Hz, 2H), 6.33 (s, 1H), 5.17 (s, 1H), 4.80-4.65 (m, 1H), 3.68 (d, J=12.2 Hz, 1H), 2.95-2.86 (m, 1H), 2.16-1.96 (m, 9H), 1.80 (ddd, J=14.0, 9.6, 5.2 Hz, 2H), 1.50 (dq, J=13.1, 6.6 Hz, 1H), 1.43-1.32 (m, 1H), 1.02 (d, J=6.5 Hz, 3H), 0.94 (d, J=6.6 Hz, 3H).

### Example 132: Preparation of Compound 235 and Compound 236

### Step 1: Methyl 6-bromoisoquinoline-1-carboxylate

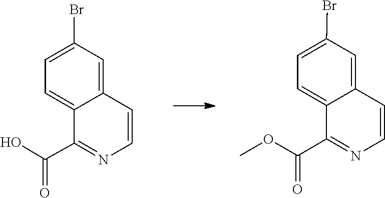

To a mixture of 6-bromoisoquinoline-1-carboxylic acid (10 g, 39.672 mmol), hydrogen chloride (33 mL of 3 M, 99.000 mmol) and MeOH (134.47 g, 170 mL, 4.1967 mol) was added sulfuric acid (3.3 mL of 18 M, 59.400 mmol) and the reaction was refluxed overnight. The reaction mixture was concentrated in vacuo. Ethyl acetate (275 mL) was added to the residue and the mixture was washed twice (50 mL) with saturated sodium bicarbonate then brine (50 mL). The organic phase was dried over sodium sulfate, filtered then concentrated to give methyl 6-bromoisoquinoline-1-carboxylate (8.54 g, 81%) as a white solid. ESI-MS m/z calc. 264.97385, found 266.0 (M+1)⁺; Retention time: 1.87 minutes; LC method K.

### Step 2: (6-Bromo-1-isoquinolyl)methanol

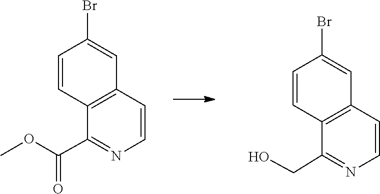

Sodium borohydride (940 mg, 24.846 mmol) was added portionwise to a solution of methyl 6-bromoisoquinoline-1-carboxylate (3 g, 11.274 mmol) in methanol (75 mL) at 0° C. under nitrogen and the reaction was stirred at room temperature overnight. The reaction was not completed after overnight. Add more sodium borohydride (430 mg, 11.366 mmol) at 0° C. then stirred at room temperature 2.5 hours. The reaction was quenched by the addition of acetone (15 mL) and the reaction was stirred for 15 minutes. The solvent was evaporated, and the residue was partitioned between water (75 mL) and ethyl acetate (200 mL). The aqueous layer was extracted with ethyl acetate (100 ml) and the combined organics were washed with brine (75 mL), dried over sodium sulfate, filtered and the solvent was evaporated. The crude material was purified by flash chromatography on silica gel, 40 g, eluted with EtOAc-Heptane 40% to 80% to give (6-bromo-1-isoquinolyl)methanol (1.49 g, 56%) as a clear oil. ESI-MS m/z calc. 236.97893, found 238.0 (M+1)⁺; Retention time: 1.2 minutes; LC method K.

### Step 3: (6-Bromo-1-isoquinolyl)methoxy-tert-butyl-dimethyl-silane

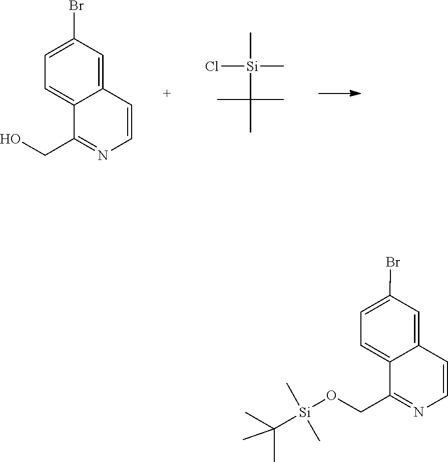

A solution of (6-bromo-1-isoquinolyl)methanol (4.55 g, 19.111 mmol) in DMF (150 mL) was treated with imidazole (1.6 g, 23.503 mmol) and tert-butyldimethylsilyl chloride (3.17 g, 21.032 mmol). The reaction was stirred at 25° C. overnight. Water (300 mL) and ethyl acetate (200 mL) was added. The organic phase was separated, and the aqueous phase was extracted with ethyl acetate (2×200 mL). The combined organic phase was washed with brine (150 mL) and dried with sodium sulfate. After filtration, the solvent was evaporated and co-evaporated with heptane to remove trace of DMF. The crude material was purified by chromatography on silica gel, 80 g, eluted with EtOAc-Heptane 10% to 30% to give (6-bromo-1-isoquinolyl)methoxy-tert-butyl-dimethyl-silane (6.73 g, 100%) as a clear oil. ¹H NMR (300 MHz, CDCl₃) δ 8.44 (d, J=5.9 Hz, 1H), 8.36 (d, J=9.1 Hz, 1H), 8.00 (d, J=2.1 Hz, 1H), 7.69 (dd, J=9.1, 2.1 Hz, 1H), 7.51 (d, J=5.6 Hz, 1H), 5.23 (s, 2H), 0.87 (s, 9H), 0.06 (s, 6H). ESI-MS m/z calc. 351.0654, found 352.1 (M+1)⁺; Retention time: 2.25 minutes; LC method K.

### Step 4: tert-Butyl-[(6-tert-butyl-1-isoquinolyl)methoxy]-dimethyl-silane

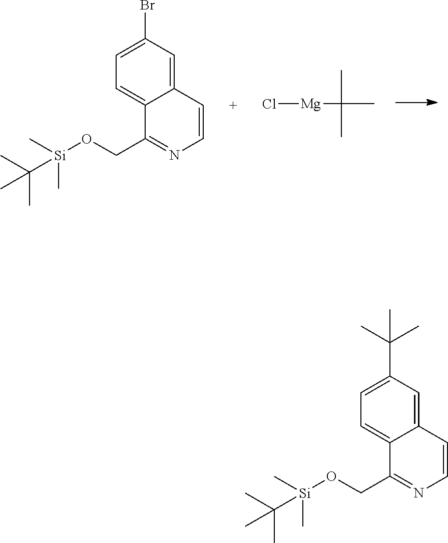

A solution of nickel chloride dihydrate (132 mg, 0.7970 mmol), 1,3-Dicyclohexylimidazolium tetrafluoroborate (200 mg, 0.6247 mmol), and (6-bromo-1-isoquinolyl)methoxy-tert-butyl-dimethyl-silane (1.4 g, 3.9734 mmol) in tetrahydrofuran (20 mL) was purged with nitrogen. The solution is cooled at 0° C. and tert-butyl(chloro)magnesium (12 mL of 1 M, 12.000 mmol) in THE was added dropwise. The solution is stirred in the cooling bath over 1 hours. The solution is poured into a stirred mixture of EtOAc (120 mL) and aqueous NH₄C₁ saturated (40 mL) and water (20 mL) and the resulting mixture is stirred for 5 minutes. The organic phase was isolated, and the aqueous phase was extracted with EtOAc (120 mL). The combine organic phases were washed with brine (60 mL) and dried over sodium sulfate, filtrated and concentrated. Give 1.8 g of crude material as a brown oil. The crude was purified by chromatography on silica gel, 80 g, eluted with EtOAc-heptane 10% to 30% to give tert-butyl-[(6-tert-butyl-1-isoquinolyl)methoxy]-dimethyl-silane (275 mg, 21%) as a clear oil. ESI-MS m/z calc. 329.2175, found 330.3 (M+1)⁺; Retention time: 2.09 minutes; LC method K.

### Step 5: tert-Butyl-[(6-tert-butyl-1,2,3,4-tetrahydroisoquinolin-1-yl)methoxy]-dimethyl-silane

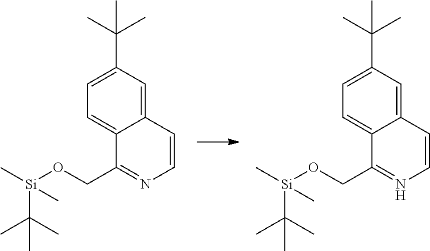 [SMILES: CC(C)(C)c1ccc2c(CO[Si](C)(C)C(C)(C)C)nccc2c1 ; CC(C)(C)c1ccc2c(CO[Si](C)(C)C(C)(C)C)nccc2c1]

To a solution of tert-butyl-[(6-tert-butyl-1-isoquinolyl)methoxy]-dimethyl-silane (750 mg, 2.2758 mmol) in ethanol (40 mL) was added platinum oxide (330 mg, 1.4532 mmol) in portions under nitrogen atmosphere. The solution was purged with hydrogen then the reaction was stirred at rt overnight. The reaction was purged with nitrogen then filtrated over Celite and concentrated. The crude material was purified by chromatography on silica gel, 40 g, eluted with EtOAc-heptane 30% to 100% to give tert-butyl-[(6-tert-butyl-1,2,3,4-tetrahydroisoquinolin-1-yl)methoxy]-dimethyl-silane (520 mg, 68%) as a clear oil. ¹H NMR (300 MHz, CDCl₃) δ 7.22-7.14 (m, 1H), 7.14-7.09 (m, 1H), 7.04 (d, J=7.9 Hz, 1H), 4.13 (q, J=7.2 Hz, 1H), 4.04 (dd, J=9.0, 3.7 Hz, 1H), 3.93-3.81 (m, 1H), 3.81-3.68 (m, 1H), 3.27-3.13 (m, 1H), 3.04-2.92 (m, 1H), 2.91-2.72 (m, 2H), 2.06 (s, 1H), 1.30 (s, 9H), 0.91 (s, 9H), 0.08 (d, J=8.5 Hz, 6H). ESI-MS m/z calc. 333.24878, found 334.3 (M+1)⁺; Retention time: 1.94 minutes; LC method K.

### Step 6: (6-tert-Butyl-1,2,3,4-tetrahydroisoquinolin-1-yl)methanol

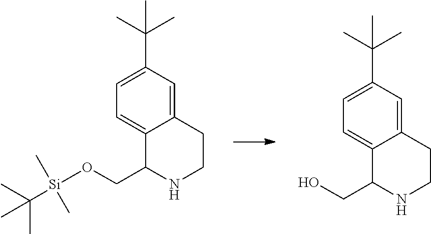 [SMILES: CC(C)(C)c1ccc2c(c1)CCNC2CO[Si](C)(C)C(C)(C)C ; CC(C)(C)c1ccc2c(c1)CCNC2CO]

To a solution of tert-butyl-[(6-tert-butyl-1,2,3,4-tetrahydroisoquinolin-1-yl)methoxy]-dimethyl-silane (720 mg, 2.1584 mmol) in THE (60 mL) was added a solution of TBAF (3.2 mL of 1 M, 3.2000 mmol) in THF. The solution was stirred at room temperature overnight. The reaction was concentrated then diluted in EtOAc (120 mL). Add water (60 mL). The organic phase was separated then the aqueous phase was extracted twice with EtOAc (15 mL). The organic phase was combined and dried over sodium sulfate. The solution was filtrated and concentrated to give 590 mg of crude (6-tert-butyl-1,2,3,4-tetrahydroisoquinolin-1-yl)methanol as a pale brown oil. ESI-MS m/z calc. 219.16231, found 220.2 (M+1)⁺; Retention time: 1.56 minutes; LC method K.

### Step 7: tert-Butyl 6-tert-butyl-1-(hydroxymethyl)-3,4-dihydro-1H-isoquinoline-2-carboxylate

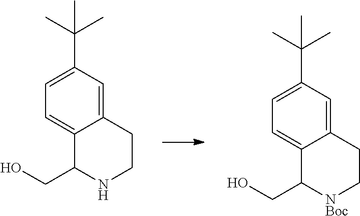 [SMILES: CC(C)(C)c1ccc2c(c1)CCNC2CO ; CC(C)(C)OC(=O)N1CCc2cc(C(C)(C)C)ccc2C1CO]

To a solution of crude (6-tert-butyl-1,2,3,4-tetrahydroisoquinolin-1-yl)methanol (200 mg, 0.9119 mmol) in dichloromethane (10 mL) was added triethylamine (217.80 mg, 0.3 mL, 2.1524 mmol) and the solution was cooled in an ice-bath. Di-tert-butyl dicarbonate (310 mg, 1.4204 mmol) was added and the mixture was stirred at room temperature overnight. The reaction was diluted with 5% aqueous sodium bicarbonate (50 mL) and the aqueous layer was extracted with dichloromethane (2×50 mL). The combined organic layers were dried over sodium sulfate, filtered and concentrated under reduced pressure. The crude was purified by chromatography on silica gel, 40 g, eluted with EtOAc-heptane 10% to 50% to gave tert-butyl 6-tert-butyl-1-(hydroxymethyl)-3,4-dihydro-1H-isoquinoline-2-carboxylate (77 mg, 26%) as a beige solid. ¹H NMR (300 MHz, CDCl₃) δ 7.23 (d, J=1.8 Hz, 1H), 7.19-7.08 (m, 2H), 5.26 (br. s., 1H), 3.86 (br. s., 3H), 3.44 (br. s., 1H), 3.00-2.67 (m, 3H), 1.49 (s, 9H), 1.30 (s, 9H). ESI-MS m/z calc. 319.21475, found 264.2 (M−56+1)⁺; Retention time: 2.17 minutes; LC method K.

### Step 8: 3-[[4-[(2-tert-Butoxycarbonyl-6-tert-butyl-3,4-dihydro-1H-isoquinolin-1-yl)methoxy]-6-(2,6-dimethylphenyl)pyrimidin-2-yl]sulfamoyl]benzoic acid

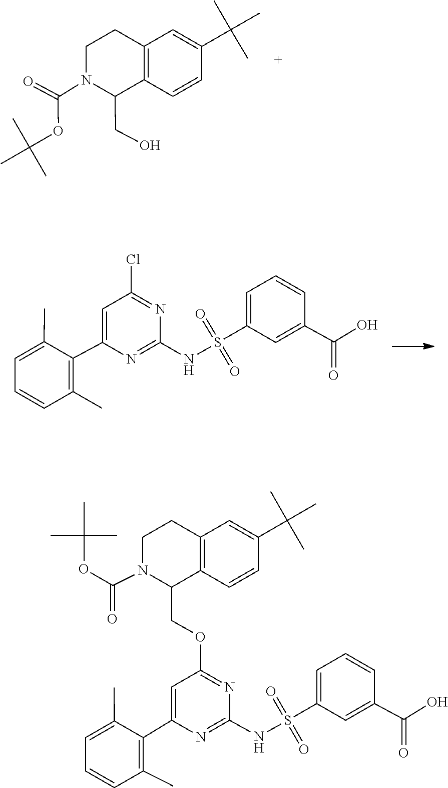

tert-Butyl 6-tert-butyl-1-(hydroxymethyl)-3,4-dihydro-1H-isoquinoline-2-carboxylate (77 mg, 0.2410 mmol) and 3-[[4-chloro-6-(2,6-dimethylphenyl)pyrimidin-2-yl]sulfamoyl]benzoic acid (110 mg, 0.2632 mmol) were dissolved in THE (2 mL). Sodium tert-butoxide (70 mg, 0.7284 mmol) were added and the reaction was stirred at room temperature overnight. Add saturated ammonium chloride solution (25 mL) and extract with EtOAc (2×30 mL). The organic phases was combined and wash with water (25 mL) and brine (15 mL) and dried over sodium sulfate. The residue was filtered and concentrated then purified by chromatography on silica gel, 12 g, eluted with EtOAc-heptane 20% to 50% to 100% follow by an other chromatography, 12 g, eluted with EtOAc-heptane 40% to 100% to gave 3-[[4-[(2-tert-butoxycarbonyl-6-tert-butyl-3,4-dihydro-1H-isoquinolin-1-yl)methoxy]-6-(2,6-dimethylphenyl)pyrimidin-2-yl]sulfamoyl]benzoic acid (23 mg, 14%) as an off-white solid. ESI-MS m/z calc. 700.2931, found 701.3 (M+1)⁺; Retention time: 2.33 minutes; LC method K. ¹H NMR (300 MHz, CDCl₃) δ 8.85-8.59 (m, 2H), 8.20 (br. s., 1H), 8.09 (br. s., 1H), 7.40-7.28 (m, 2H), 7.24-7.14 (m, 2H), 7.09 (t, J=8.4 Hz, 3H), 6.13 (d, J=6.2 Hz, 1H), 5.55 (d, J=15.9 Hz, 1H), 4.61 (br. s., 1H), 4.42-4.21 (m, 1H), 4.00 (d, J=13.2 Hz, 1H), 3.51-3.30 (m, 1H), 3.28-3.10 (m, 1H), 3.02-2.85 (m, 1H), 2.82-2.70 (m, 1H), 2.15-2.02 (m, 6H), 1.50-1.35 (m, 9H), 1.32 (s, 9H).

### Step 9: 3-[[4-[(6-tert-Butyl-1,2,3,4-tetrahydroisoquinolin-1-yl)methoxy]-6-(2,6-dimethylphenyl)pyrimidin-2-yl]sulfamoyl]benzoic acid

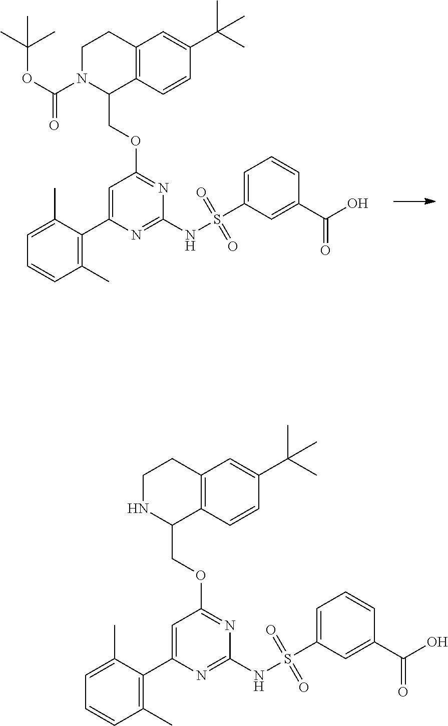 [SMILES: Cc1cccc(C)c1-c1cc(OCC2c3ccc(C(C)(C)C)cc3CCN2C(=O)OC(C)(C)C)nc(NS(=O)(=O)c2cccc(C(=O)O)c2)n1 ; Cc1cccc(C)c1-c1cc(OCC2NCCc3cc(C(C)(C)C)ccc32)nc(NS(=O)(=O)c2cccc(C(=O)O)c2)n1]

3-[[4-[(2-tert-Butoxycarbonyl-6-tert-butyl-3,4-dihydro-1H-isoquinolin-1-yl)methoxy]-6-(2,6-dimethylphenyl)pyrimidin-2-yl]sulfamoyl]benzoic acid (23 mg, 0.0328 mmol) was dissolved in dioxane (2 mL). The solution was treated with hydrogen chloride (0.2 mL of 4 M, 0.8000 mmol) and the reaction was stirred at room temperature overnight. Not completed. Add hydrogen chloride (0.5 mL of 4 M, 2.0000 mmol) and stirred at room temperature 2 hours. Reaction progress. Stirred 2 hours again. No progress. Add hydrogen chloride (0.5 mL of 4 M, 2.0000 mmol). Stirred at room temperature 30 minutes. The reaction was concentrated to give crude 3-[[4-[(6-tert-butyl-1,2,3,4-tetrahydroisoquinolin-1-yl)methoxy]-6-(2,6-dimethylphenyl)pyrimidin-2-yl]sulfamoyl]benzoic acid (hydrochloride salt) (20.9 mg, 100%) as a white solid. ESI-MS m/z calc. 600.2406, found 601.3 (M+1)⁺; Retention time: 1.82 minutes. LC method K.

### Step 10: 8-tert-Butyl-17-(2,6-dimethylphenyl)-14-oxa-21λ⁶-thia-3,18,20,27-tetraazapentacyclo[20.3.1.115,19.03,12.06,11]heptacosa-1(26),6(11),7,9,15(27),16,18,22,24-nonaene-2,21,21-trione

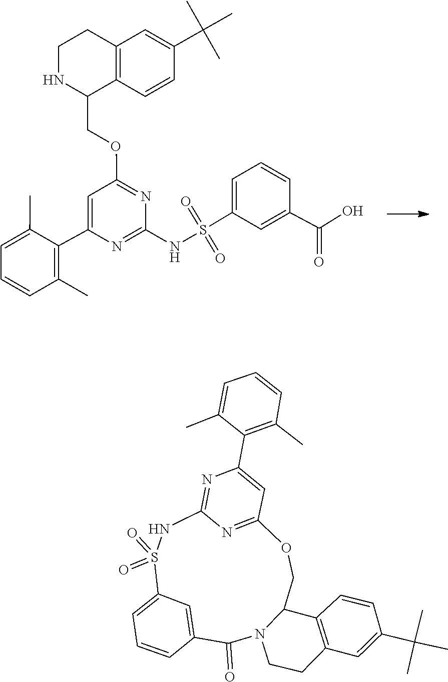 [SMILES: Cc1cccc(C)c1-c1cc(OCC2NCCc3cc(C(C)(C)C)ccc32)nc(NS(=O)(=O)c2cccc(C(=O)O)c2)n1 ; Cc1cccc(C)c1-c1cc2nc(n1)NS(=O)(=O)c1cccc(c1)C(=O)N1CCc3cc(C(C)(C)C)ccc3C1CO2]

The crude 3-[[4-[(6-tert-butyl-1,2,3,4-tetrahydroisoquinolin-1-yl)methoxy]-6-(2,6-dimethylphenyl)pyrimidin-2-yl]sulfamoyl]benzoic acid (hydrochloride salt) (21 mg, 0.0330 mmol) was dissolved in DMF (2 mL). Add triethylamine (36.300 mg, 50 μL, 0.3587 mmol) and HATU (14 mg, 0.0368 mmol). Stir at room temperature overnight. The reaction was diluted with water (25 mL) and EtOAc (30 mL). The organic phase was separated, and the aqueous phase was extracted with EtOAc (25 mL). The organic phases was combined and washed with water (20 mL) and brine (15 mL). The organic phase was dried over sodium sulfate, filtered and concentrated. The residue was purified by chromatography on silica gel, 4 g, eluted with EtOAc-Heptane 30% to 70% to gave racemic 8-tert-butyl-17-(2,6-dimethylphenyl)-14-oxa-21λ⁶-thia-3,18,20,27-tetraazapentacyclo[20.3.1.115,19.03,12.06,11]heptacosa-1(26),6(11),7,9,15(27),16,18,22,24-nonaene-2,21,21-trione (7.24 mg, 34%) as an off white solid. ESI-MS m/z calc. 582.2301, found 583.2 (M+1)⁺; Retention time: 3.41 minutes; LC method U. ¹H NMR (300 MHz, CDCl₃) δ 8.77 (s, 1H), 7.86 (d, J=7.6 Hz, 1H), 7.74 (d, J=7.6 Hz, 1H), 7.56 (t, J=7.8 Hz, 1H), 7.25-7.18 (m, 2H), 7.11 (d, J=7.6 Hz, 2H), 7.03 (d, J=8.5 Hz, 1H), 6.48 (d, J=8.2 Hz, 1H), 6.35 (s, 1H), 5.62 (dd, J=10.7, 4.0 Hz, 1H), 4.81-4.73 (m, 1H), 4.69 (dd, J=12.2, 4.6 Hz, 1H), 4.37 (t, J=10.9 Hz, 1H), 3.83-3.66 (m, 1H), 3.44-3.31 (m, 1H), 3.29-3.15 (m, 1H), 2.91 (d, J=16.1 Hz, 1H), 2.14 (s, 6H), 1.30-1.23 (s, 9H).

### Step 11: 8-tert-Butyl-17-(2,6-dimethylphenyl)-14-oxa-21λ⁶-thia-3,18,20,27-tetraazapentacyclo[20.3.1.115,19.03,12.06,11]heptacosa-1(26),6(11),7,9,15(27),16,18,22,24-nonaene-2,21,21-trione, enantiomer 1 (Compound 236), and 8-tert-butyl-17-(2,6-dimethylphenyl)-14-oxa-21λ⁶-thia-3,18,20,27-tetraazapentacyclo[20.3.1.115,19.03,12.06,11]heptacosa-1(26),6(11),7,9,15(27),16,18,22,24-nonaene-2,21,21-trione, enantiomer 2 (Compound 235)

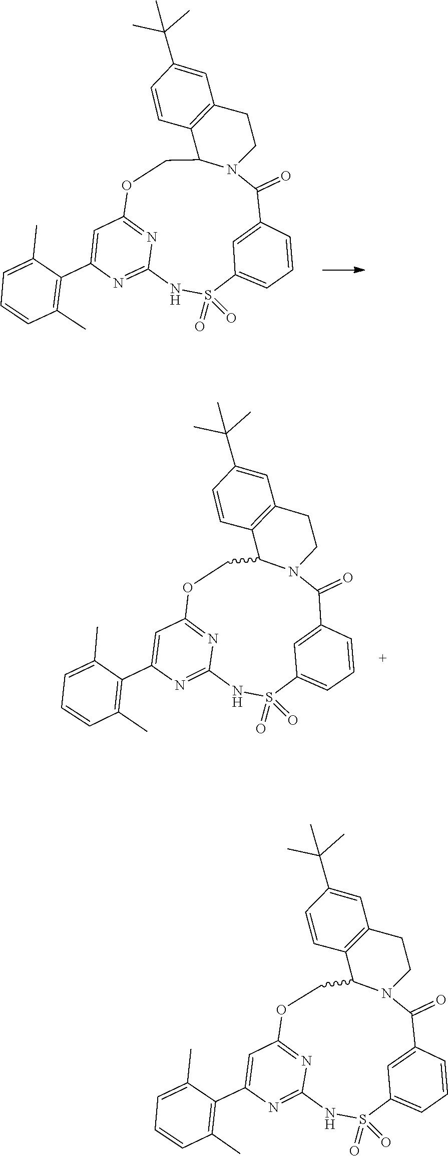 [SMILES: Cc1cccc(C)c1-c1cc2nc(n1)NS(=O)(=O)c1cccc(c1)C(=O)N1CCc3cc(C(C)(C)C)ccc3C1CO2 ; Cc1cccc(C)c1-c1cc2nc(n1)NS(=O)(=O)c1cccc(c1)C(=O)N1CCc3cc(C(C)(C)C)ccc3C1CO2 ; Cc1cccc(C)c1-c1cc2nc(n1)NS(=O)(=O)c1cccc(c1)C(=O)N1CCc3cc(C(C)(C)C)ccc3C1CO2]

Racemic 8-tert-Butyl-17-(2,6-dimethylphenyl)-14-oxa-21λ⁶-thia-3,18,20,27-tetraazapentacyclo[20.3.1.115,19.03,12.06,11]heptacosa-1(26),6(11),7,9,15(27),16,18,22,24-nonaene-2,21,21-trione (7.2 mg, 0.01236 mmol) was purified using a normal phase SFC-MS method using a AS-H column (250×21.2 mm, 5 μm particle size) sold by Chiral Technologies (pn: 20945), and a dual gradient run from 5-40% mobile phase B over 17.5 minutes. Mobile phase A=CO₂. Mobile phase B=MeOH (20 mM NH₃). Flow rate=5-15% MeOH [20 mM NH₃] 80 mL/min, 15-80% MeOH [20 mM NH₃] 40 mL/min. injection volume=variable, and column temperature=40° C. to give enantiomer 1, 8-tert-butyl-17-(2,6-dimethylphenyl)-14-oxa-21λ⁶-thia-3,18,20,27-tetraazapentacyclo[20.3.1.115,19.03,12.06,11]heptacosa-1(26),6(11),7,9,15(27),16,18,22,24-nonaene-2,21,21-trione (2.7 mg, 75%) ESI-MS m/z calc. 582.2301, found 583.0 (M+1)⁺; Retention time: 2.79 minutes; LCMS LC method I, and enantiomer 2, 8-tert-butyl-17-(2,6-dimethylphenyl)-14-oxa-21λ⁶-thia-3,18,20,27-tetraazapentacyclo[20.3.1.115,19.03,12.06,11]heptacosa-1(26),6(11),7,9,15(27),16,18,22,24-nonaene-2,21,21-trione (2.8 mg, 77%) ESI-MS m/z calc. 582.2301, found 583.0 (M+1)⁺; Retention time: 2.79 minutes; LC method I.

### Example 133: Preparation of Compound 237

### Step 1: (2R)-4-Methyl-2-(p-tolylsulfonylamino)pentanoic acid

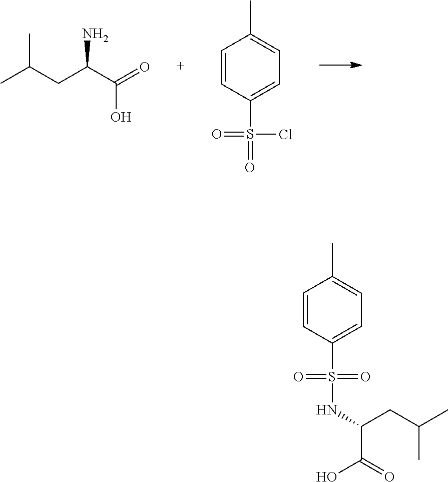

(2R)-2-Amino-4-methyl-pentanoic acid (18 g, 137.22 mmol) was dissolved in water (280 mL) and NaOH (16.6 g, 415.03 mmol) was added. Once all the solids were dissolved, 4-methylbenzenesulfonyl chloride (31.5 g, 165.23 mmol) was added and the reaction warmed to 60° C. for 3 days. The reaction became a clear solution. After three days, the reaction was cooled to 0° C. and concentrated HCl was added until pH 1 was reached. The reaction was allowed to stir for 30 minutes at 0° C. and the precipitate was filtered to give (2R)-4-methyl-2-(p-tolylsulfonylamino)pentanoic acid (21 g, 51%) as a white solid. ¹H NMR (250 MHz, CDCl₃) δ 7.74 (d, J=7.9 Hz, 2H), 7.28 (d, J=7.9 Hz, 2H), 5.18-5.01 (m, 1H), 4.00-3.80 (m, 1H), 2.41 (s, 3H), 1.83-1.66 (m, 1H), 1.59-1.43 (m, 2H), 0.85 (dd, J=18.6, 6.5 Hz, 6H).

### Step 2: (2R)—N-Methoxy-N,4-dimethyl-2-(p-tolylsulfonylamino)pentanamide

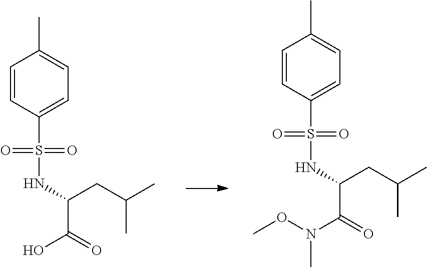 [SMILES: Cc1ccc(S(=O)(=O)N[C@H](CC(C)C)C(=O)O)cc1 ; CON(C)C(=O)[C@@H](CC(C)C)NS(=O)(=O)c1ccc(C)cc1]

(2R)-4-Methyl-2-(p-tolylsulfonylamino)pentanoic acid (21 g, 73.592 mmol) N-methoxymethanamine (hydrochloride salt) (10.9 g, 111.74 mmol) and DIPEA (38.1 g, 51.348 mL, 294.79 mmol) were dissolved in DMF (370 mL) and HATU (56.01 g, 147.31 mmol) was added. This was stirred for 15 minutes then water (800 mL) was added. The organic layer was extracted with EtOAc (350 mL) then the combined organic layers were washed with brine (5×100 mL) and dried over sodium sulfate then concentrated. The crude residue was dry loaded on to silica gel and purified by flash column chromatography using 0-40% hexanes:EtOAc as an eluent. The appropriate fractions were collected to give (2R)—N-methoxy-N,4-dimethyl-2-(p-tolylsulfonylamino)pentanamide (18.6 g, 73%) as a white solid. ESI-MS m/z calc. 328.1457, found 329.5 (M+1)⁺; Retention time: 2.96 minutes; LC method T.

### Step 3: tert-Butyl N-[(1R)-1-[methoxy(methyl)carbamoyl]-3methyl-butyl]-N-(p-tolylsulfonyl)carbamate

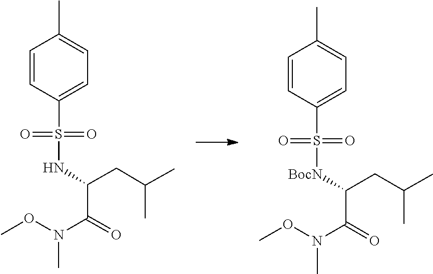 [SMILES: CON(C)C(=O)[C@@H](CC(C)C)NS(=O)(=O)c1ccc(C)cc1 ; CON(C)C(=O)[C@@H](CC(C)C)N(C(=O)OC(C)(C)C)S(=O)(=O)c1ccc(C)cc1]

Into a solution of (2R)—N-methoxy-N,4-dimethyl-2-(p-tolylsulfonylamino)pentanamide (5.07 g, 15.437 mmol) in ACN (50 mL) was added tert-butoxycarbonyl tert-butyl carbonate (5.2 g, 23.826 mmol) and DMAP (220 mg, 1.8008 mmol). The reaction was stirred at room temperature for 1 hour, then it was concentrated under vacuum. The residue was purified by silica gel chromatography using 0 to 50% ethyl acetate in hexane (120 g column) to furnish tert-butyl N-[(1R)-1-[methoxy(methyl)carbamoyl]-3-methyl-butyl]-N-(p-tolylsulfonyl)carbamate (6.75 g, 100%) as a clear oil. ESI-MS m/z calc. 428.19812, found 429.2 (M+1)⁺; Retention time: 5.83 minutes; LC method S.

### Step 4: tert-Butyl N-[(1R)-1-formyl-3-methyl-butyl]-N-(p-tolylsulfonyl)carbamate

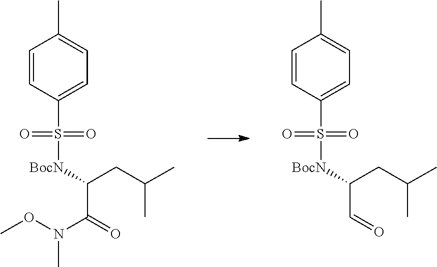 [SMILES: CON(C)C(=O)[C@@H](CC(C)C)N(C(=O)OC(C)(C)C)S(=O)(=O)c1ccc(C)cc1 ; Cc1ccc(S(=O)(=O)N(C(=O)OC(C)(C)C)[C@@H](C=O)CC(C)C)cc1]

Into a solution of tert-butyl N-[(1R)-1-[methoxy(methyl)carbamoyl]-3-methyl-butyl]-N-(p-tolylsulfonyl)carbamate (8.98 g, 20.955 mmol) in anhydrous DCM (100 mL) was added DIBAL-H (42 mL of 1 M, 42.000 mmol) in toluene at −78° C. The reaction was stirred at the same temperature for 1 minutes, then it was quenched with methanol (10 mL). The reaction was warmed up to room temperature. Saturated potassium sodium tartrate (100 mL) was added. The reaction mixture was stirred for 1 hour until both layers became clear. Two layers were separated. The aqueous layer was extracted with DCM (2×100 mL). The combined organic layers were washed with brine (10 mL), dried over anhydrous sodium sulfate and concentrated under vacuum to furnish tert-butyl N-[(1R)-1-formyl-3-methyl-butyl]-N-(p-tolylsulfonyl)carbamate (8.94 g, 88%) as a white solid. ESI-MS m/z calc. 369.16098, found 370.2 (M+1)⁺; Retention time: 5.44 minutes; LC method S.

### Step 5: 1-Bromo-4-tert-butyl-2-iodo-benzene

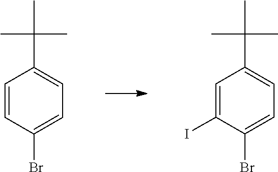

To a solution of 1-bromo-4-tert-butyl-benzene (150 g, 703.85 mmol) in TFA (1.5000 L) was added NIS (166.27 g, 739.04 mmol) portion-wise at room temperature. The reaction was allowed to stir for 6 hours before the volatiles were removed by evaporation. The crude residue was diluted with water (800 mL) and EtOAc (800 mL). The aqueous layer was extracted three times with EtOAc (3×1 L). The combined organic layers were washed with sodium bicarbonate (1 L), water (1 L), and brine (1 L), then dried over sodium sulfate and concentrated under vacuum. This crude residue was dissolved in hexanes and passed through a pad of silica gel. The pad of silica gel was washed three times with hexanes (3×800 mL), this residue was concentrated to give 1-bromo-4-tert-butyl-2-iodo-benzene (235.93 g, 99%). ¹H NMR (250 MHz, CDCl₃) δ 7.84 (d, J=2.2 Hz, 1H), 7.68-7.44 (m, 1H), 7.23-7.07 (m, 1H), 1.27 (s, 9H). ESI-MS m/z calc. 337.91672, no ionization observed, Retention time: 3.92 minutes; LC method T.

### Step 6: 1-Bromo-4-tert-butyl-2-vinyl-benzene

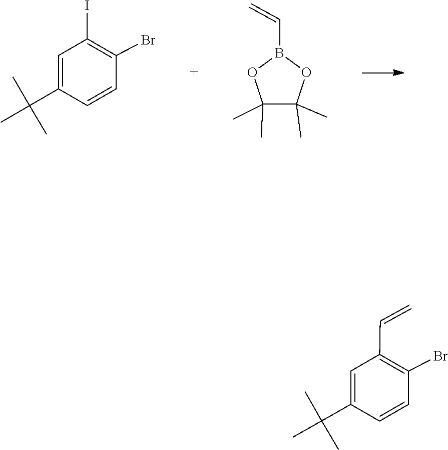

1-Bromo-4-tert-butyl-2-iodo-benzene (53.7 g, 158.40 mmol) and vinylboronic acid pinacol ester (29.36 g, 190.63 mmol) were dissolved in DMF (735 mL) and potassium carbonate (65.87 g, 476.61 mmol) was added. The solution was degassed for 10 minutes then Pd(dppf)Cl₂ (5.85 g, 7.9950 mmol) was added and the reaction warmed to 60° C. overnight. The reaction was quenched with water (150 mL) then concentrated to a volume of ˜300 mL. More water was added (700 mL) and the aqueous layer was extracted with EtOAc (3×200 mL). The combined organic layers were washed with brine (5×100 mL) then dried over sodium sulfate and concentrated. The crude residue was purified by a silica plug eluting with 5% hexane:EtOAc to give 1-bromo-4-tert-butyl-2-vinyl-benzene (30.63 g, 77%) as a yellow oil. ¹H NMR (250 MHz, CDCl₃) δ 7.57 (s, 1H), 7.47 (d, J=8.5 Hz, 1H), 7.17 (d, J=8.5 Hz, 1H), 7.06 (dd, J=17.5, 11.0 Hz, 1H), 5.71 (d, J=17.5 Hz, 1H), 5.36 (d, J=11.0 Hz, 1H), 1.33 (s, 9H).

### Step 7: 2-(2-Bromo-5-tert-butyl-phenyl)ethanol

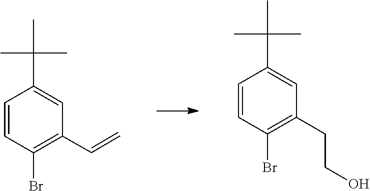

1-Bromo-4-tert-butyl-2-vinyl-benzene (30.63 g, 96.058 mmol) was dissolved in THE (100 mL) and cooled to 0° C. Borane dimethyl sulfide complex (48.000 mL of 2 M, 96.000 mmol) was added slowly and the reaction was allowed to stir overnight. The reaction was cooled to 0° C. then water (75 mL) was added slowly. NaOH (58 mL of 2 M, 116.00 mmol) and hydrogen peroxide (110 g, 30% w/w, 970.17 mmol) were then added and the reaction was warmed to room temperature and stirred for 1 h 45 minutes. The layers were separated, and the aqueous layer was extracted three times with DCM (50 mL). The organic layers were dried over sodium sulfate and concentrated. The crude residue was dry loaded on to silica gel and purified by flash column chromatography using 0-30% hexane:EtOAc as an eluent to give 2-(2-bromo-5-tert-butyl-phenyl)ethanol (16.31 g, 63%) as a colorless oil. ¹H NMR (250 MHz, CDCl₃) δ 7.47 (d, J=8.4 Hz, 1H), 7.32-7.23 (m, 1H), 7.19-7.07 (m, 1H), 3.89 (t, J=6.9 Hz, 2H), 3.03 (t, J=6.9 Hz, 2H), 1.30 (s, 9H).

### Step 8: 2-(2-Bromo-5-tert-butyl-phenyl)ethoxy-tert-butyl-dimethyl-silane

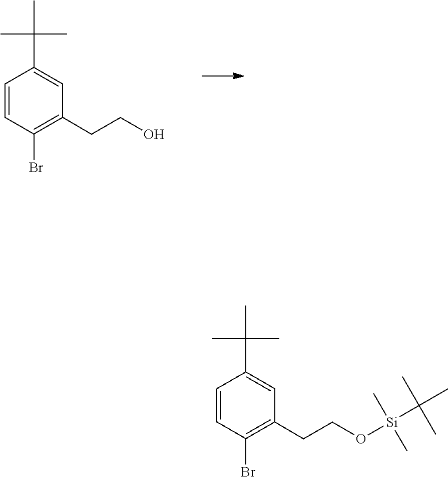 [SMILES: CC(C)(C)c1ccc(Br)c(CCO)c1 ; CC(C)(C)c1ccc(Br)c(CCO[Si](C)(C)C(C)(C)C)c1]

2-(2-Bromo-5-tert-butyl-phenyl)ethanol (16.31 g, 63.422 mmol) was dissolved in DMF (250 mL) then imidazole (8.69 g, 127.65 mmol) and TBSCl (19.07 g, 126.52 mmol) were added and the reaction was stirred at room temperature overnight. The reaction was concentrated to ˜50 mL of solvent, then diluted with 500 mL of water. The organic layer was extracted three times with EtOAc (125 mL) then the organic layer was washed three times with brine (50 mL). The organic lay was dried over sodium sulfate and concentrated. The crude residue was dry loaded on to silica gel and purified by flash column chromatography using 0-20% hexanes:EtOAc as an eluent to give 2-(2-bromo-5-tert-butyl-phenyl)ethoxy-tert-butyl-dimethyl-silane (18.45 g, 74%) as a colorless oil. ¹H NMR (250 MHz, CDCl₃) δ 7.43 (d, J=8.4 Hz, 1H), 7.27 (s, 1H), 7.10 (dd, J=8.4, 2.5 Hz, 1H), 3.84 (t, J=7.0 Hz, 1H), 2.96 (t, J=7.0 Hz, 1H), 1.29 (s, 9H), 0.87 (s, 9H), −0.02 (s, 6H).

### Step 9: tert-Butyl N-[(1R)-1-[[4-tert-butyl-2-[2-[tert-butyl(dimethyl)silyl]oxyethyl]phenyl]-hydroxy-methyl]-3-methyl-butyl]-N-(p-tolylsulfonyl)carbamate

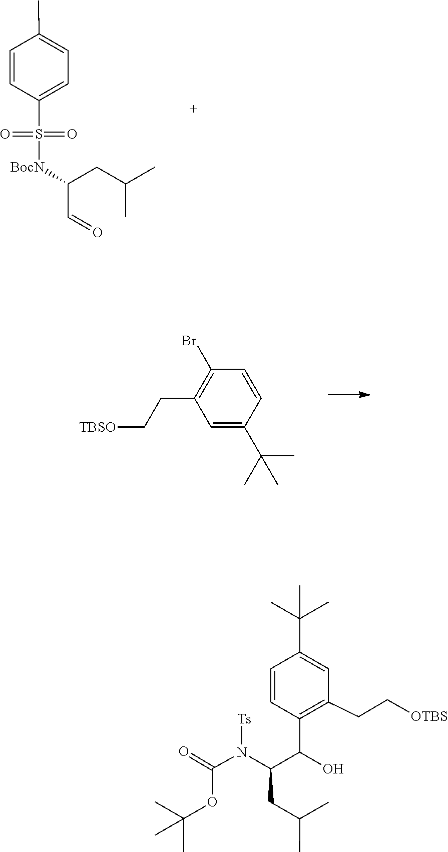 [SMILES: Cc1ccc(S(=O)(=O)N(C(=O)OC(C)(C)C)[C@@H](C=O)CC(C)C)cc1 ; CC(C)(C)c1ccc(Br)c(CCO[Si](C)(C)C(C)(C)C)c1 ; Cc1ccc(S(=O)(=O)N(C(=O)OC(C)(C)C)[C@H](CC(C)C)C(O)c2ccc(C(C)(C)C)cc2CCO[Si](C)(C)C(C)(C)C)cc1]

Into a solution of 2-(2-bromo-5-tert-butyl-phenyl)ethoxy-tert-butyl-dimethyl-silane (8.24 g, 22.185 mmol) in anhydrous THE (100 mL) was added nBuLi (9 mL of 2.5 M, 22.500 mmol) in hexane drop-wise at −78° C. The reaction was stirred at the same temperature for 1 hour. A solution of tert-butyl N-[(1R)-1-formyl-3-methyl-butyl]-N-(p-tolylsulfonyl)carbamate (8.94 g, 18.389 mmol) in anhydrous THE (50 mL) was added to the reaction mixture drop-wise at −78° C. The reaction mixture was stirred at the same temperature for 0.5 hour. The reaction was quenched with saturated ammonium chloride (150 mL) at −78° C. After warmed up to room temperature, saturated ammonium chloride (150 mL) were added. The two layers were separated, and the aqueous layer was extracted with ethyl acetate (2×150 mL). The combined organic layers were washed with brine (150 mL), dried over anhydrous sodium sulfate and concentrated under vacuum. The residue was purified by silica gel chromatography using 0 to 30% ethyl acetate in hexane to furnish tert-butyl N-[(1R)-1-[[4-tert-butyl-2-[2-[tert-butyl(dimethyl)silyl]oxyethyl]phenyl]-hydroxy-methyl]-3-methyl-butyl]-N-(p-tolylsulfonyl)carbamate (10.34 g, 79%) as a white foamy solid. ESI-MS m/z calc. 661.38324, found 662.4 (M+1)⁺; Retention time: 8.71 minutes; LC method S.

### Step 10: [(2R)-2-[tert-Butoxycarbonyl(p-tolylsulfonyl)amino]-1-[4-tert-butyl-2-[2-[tert-butyl(dimethyl)silyl]oxyethyl]phenyl]-4-methyl-pentyl]acetate

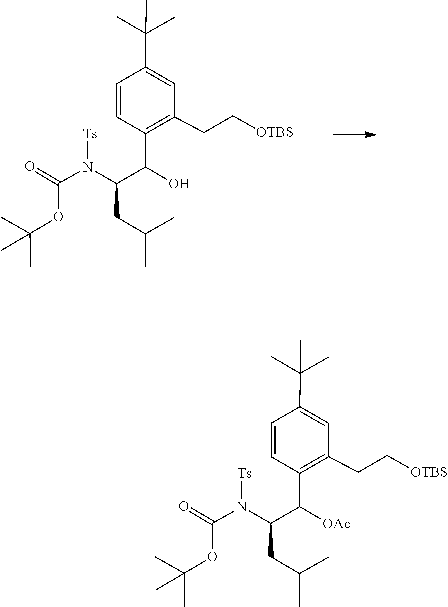 [SMILES: Cc1ccc(S(=O)(=O)N(C(=O)OC(C)(C)C)[C@H](CC(C)C)C(O)c2ccc(C(C)(C)C)cc2CCO[Si](C)(C)C(C)(C)C)cc1 ; CC(=O)OC(c1ccc(C(C)(C)C)cc1CCO[Si](C)(C)C(C)(C)C)[C@@H](CC(C)C)N(C(=O)OC(C)(C)C)S(=O)(=O)c1ccc(C)cc1]

Into a solution of tert-butyl N-[(1R)-1-[[4-tert-butyl-2-[2-[tert-butyl(dimethyl)silyl]oxyethyl]phenyl]-hydroxy-methyl]-3-methyl-butyl]-N-(p-tolylsulfonyl)carbamate (9.75 g, 14.728 mmol) in anhydrous DMF (100 mL) was added acetyl chloride (3.5328 g, 3.2 mL, 45.005 mmol). NaH (5.9 g, 60% w/w, 147.51 mmol) was added to the reaction mixture at 0° C. The reaction was stirred at room temperature for 2 hours. The reaction was quenched with saturated ammonium chloride (200 mL) and diluted with diethyl ether (200 mL). Two layers were separated, and the aqueous layer was extracted with diethyl ether (2×200 mL). The combined ether layers were washed with brine (3×100 mL), dried over anhydrous sodium sulfate and concentrated under vacuum. The residue was purified by silica gel chromatography using 0 to 50% diethyl ether in hexane to furnish [(2R)-2-[tert-butoxycarbonyl(p-tolylsulfonyl)amino]-1-[4-tert-butyl-2-[2-[tert-butyl(dimethyl)silyl]oxyethyl]phenyl]-4-methyl-pentyl]acetate (1.97 g, 19%) as a clear gel. ESI-MS m/z calc. 703.3938, found 721.5 (M+18)⁺; Retention time: 8.99 minutes (mixture of diastereomers) (LC method S). A significant amount of starting material tert-butyl N-[(1R)-1-[[4-tert-butyl-2-[2-[tert-butyl(dimethyl)silyl]oxyethyl]phenyl]-hydroxy-methyl]-3-methyl-butyl]-N-(p-tolylsulfonyl)carbamate (7.21 g, 74%) was recovered.

### Step 11: [(2R)-1-[4-tert-Butyl-2-(2-hydroxyethyl)phenyl]-4-methyl-2-(p-tolylsulfonylamino)pentyl]acetate, diastereomer 1 and diastereomer 2

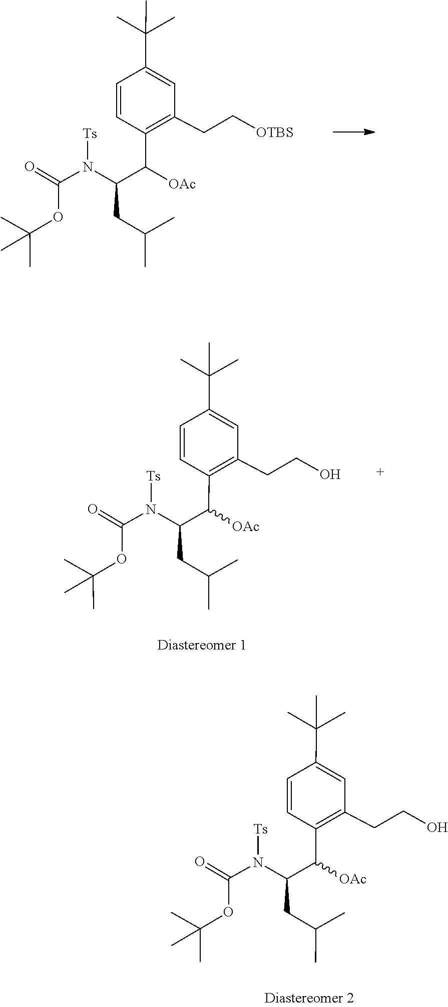 [SMILES: CC(=O)OC(c1ccc(C(C)(C)C)cc1CCO[Si](C)(C)C(C)(C)C)[C@@H](CC(C)C)N(C(=O)OC(C)(C)C)S(=O)(=O)c1ccc(C)cc1 ; CC(=O)OC(c1ccc(C(C)(C)C)cc1CCO)[C@@H](CC(C)C)N(C(=O)OC(C)(C)C)S(=O)(=O)c1ccc(C)cc1 ; CC(=O)OC(c1ccc(C(C)(C)C)cc1CCO)[C@@H](CC(C)C)N(C(=O)OC(C)(C)C)S(=O)(=O)c1ccc(C)cc1]

[(2R)-2-[tert-Butoxycarbonyl(p-tolylsulfonyl)amino]-1-[4-tert-butyl-2-[2-[tert-butyl(dimethyl)silyl]oxyethyl]phenyl]-4-methyl-pentyl]acetate (1.97 g, 2.7981 mmol) was dissolved into a solution of HCl (20 mL of 4 M, 80.000 mmol) in dioxane at 0° C. The reaction was stirred at room temperature for 1.5 hours. The reaction was quenched with saturated sodium bicarbonate (100 mL) and extracted with diethyl ether (3×100 mL). The combined ether layer was washed with brine (100 mL), dried over anhydrous sodium sulfate and concentrated under vacuum. The residue was purified by silica gel chromatography using 50 to 100% diethyl ether in hexane (40 g column) to furnish isomer A (less polar), diastereomer 1 [(2R)-1-[4-tert-butyl-2-(2-hydroxyethyl)phenyl]-4-methyl-2-(p-tolylsulfonylamino)pentyl]acetate (0.13 g, 9%) as a clear oil, and isomer B (more polar), diastereomer 2 [(2R)-1-[4-tert-butyl-2-(2-hydroxyethyl)phenyl]-4-methyl-2-(p-tolylsulfonylamino)pentyl]acetate (0.71 g, 52%) as a white solid. Isomer A, diastereomer 1: ESI-MS m/z calc. 489.2549, found 507.2 (M+18)⁺; Retention time: 6.05 minutes (LC method S), ¹H NMR (250 MHz, Chloroform-d) δ 7.79-7.66 (m, 2H), 7.34-7.21 (m, 2H), 7.19-7.08 (m, 2H), 7.05 (d, J=8.8 Hz, 1H), 5.99 (d, J=3.7 Hz, 1H), 5.27 (d, J=8.7 Hz, 1H), 3.94-3.79 (m, 1H), 3.79-3.73 (m, 1H), 3.72-3.58 (m, 1H), 2.86 (h, J=7.3 Hz, 2H), 2.42 (s, 3H), 2.02 (s, 3H), 1.59-1.47 (m, 1H), 1.39-1.30 (m, 2H), 1.27 (s, 9H), 0.81 (d, J=6.6 Hz, 3H), 0.53 (d, J=6.5 Hz, 3H); and isomer B, diastereomer 2: ESI-MS m/z calc. 489.2549, found 507.6 (M+18)⁺; Retention time: 5.98 minutes (LC method S), ¹H NMR (250 MHz, CDCl₃) δ 7.61 (d, J=8.3 Hz, 2H), 7.24-7.12 (m, 5H), 6.01 (d, J=5.5 Hz, 1H), 4.61 (d, J=9.3 Hz, 1H), 3.83 (m, 3H), 3.04-2.66 (m, 2H), 2.39 (s, 3H), 1.96 (s, 3H), 1.54-1.44 (m, 1H), 1.30 (s, 9H), 1.25 (dd, J=7.0, 1.8 Hz, 2H), 0.81 (d, J=6.6 Hz, 3H), 0.71 (d, J=6.4 Hz, 3H).

### Step 12: [(2R)-7-tert-Butyl-2-isobutyl-3-(p-tolylsulfonyl)-1,2,4,5-tetrahydro-3-benzazepin-1-yl]acetate, diastereomer 2

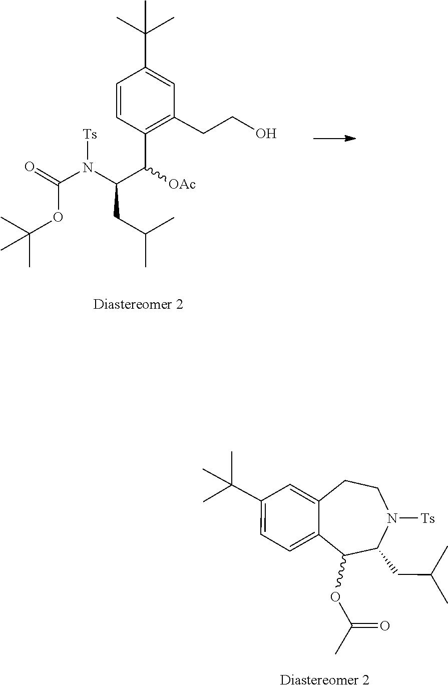 [SMILES: CC(=O)OC(c1ccc(C(C)(C)C)cc1CCO)[C@@H](CC(C)C)N(C(=O)OC(C)(C)C)S(=O)(=O)c1ccc(C)cc1 ; CC(=O)OC1c2ccc(C(C)(C)C)cc2CCN(S(=O)(=O)c2ccc(C)cc2)[C@@H]1CC(C)C]

Into a solution of [(2R)-1-[4-tert-butyl-2-(2-hydroxyethyl)phenyl]-4-methyl-2-(p-tolylsulfonylamino)pentyl]acetate, diastereomer 2 (1.472 g, 3.0061 mmol) and triphenylphosphine (1.59 g, 6.0621 mmol) in anhydrous THE (15 mL) was added DIAD (908.28 mg, 0.87 mL, 4.4918 mmol) drop-wise at 0° C. The reaction was stirred at room temperature for 1 hours. The solvent was removed under vacuum. The residue was purified by silica gel chromatography using 0 to 20% ethyl acetate in hexane (40 g silica gel column) to furnish [(2R)-7-tert-butyl-2-isobutyl-3-(p-tolylsulfonyl)-1,2,4,5-tetrahydro-3-benzazepin-1-yl]acetate, diastereomer 2 (1.323 g, 93%) as a white solid. ESI-MS m/z calc. 471.24432, found 472.3 (M+1)⁺; Retention time: 7.15 minutes; LC method S.

### Step 13: (2R)-7-tert-Butyl-2-isobutyl-3-(p-tolylsulfonyl)-1,2,4,5-tetrahydro-3-benzazepin-1-ol, diastereomer 2

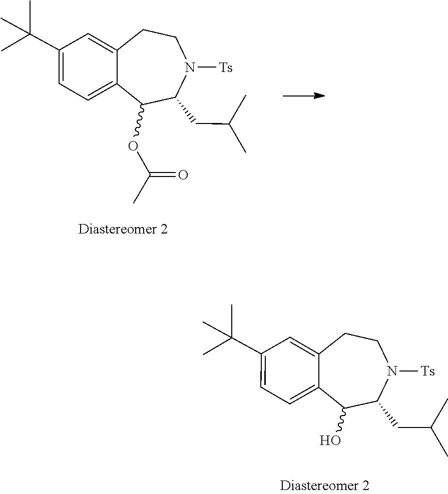 [SMILES: CC(=O)OC1c2ccc(C(C)(C)C)cc2CCN(S(=O)(=O)c2ccc(C)cc2)[C@@H]1CC(C)C ; Cc1ccc(S(=O)(=O)N2CCc3cc(C(C)(C)C)ccc3C(O)[C@H]2CC(C)C)cc1]

Into a solution of [(2R)-7-tert-butyl-2-isobutyl-3-(p-tolylsulfonyl)-1,2,4,5-tetrahydro-3-benzazepin-1-yl]acetate, diastereomer 2 (1.323 g, 2.8050 mmol) in methanol (50 mL) was added potassium carbonate (577 mg, 4.1749 mmol). The reaction was stirred at room temperature for an hour. The reaction concentrated under vacuum, and then it was diluted with diethyl ether (100 mL) and 1 N HCl (aq.) (100 mL). Two layers were separated. The aqueous layer was extracted with diethyl ether (50 mL). The combined organic layers were washed with brine (50 mL), dried over anhydrous magnesium sulfate and concentrated under vacuum to furnish (2R)-7-tert-butyl-2-isobutyl-3-(p-tolylsulfonyl)-1,2,4,5-tetrahydro-3-benzazepin-1-ol, diastereomer 2 (1.086 g, 90%) as a white solid, which was used in the next step reaction without purification. ESI-MS m/z calc. 429.23376, found 430.5 (M+1)⁺; Retention time: 6.62 minutes; LC method S.

### Step 14: (2R)-7-tert-Butyl-2-isobutyl-2,3,4,5-tetrahydro-1H-3-benzazepin-1-ol, diastereomer 2

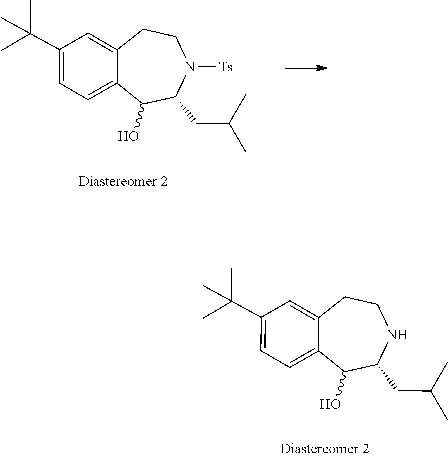 [SMILES: Cc1ccc(S(=O)(=O)N2CCc3cc(C(C)(C)C)ccc3C(O)[C@H]2CC(C)C)cc1 ; CC(C)C[C@H]1NCCc2cc(C(C)(C)C)ccc2C1O]

Into a solution of (2R)-7-tert-butyl-2-isobutyl-3-(p-tolylsulfonyl)-1,2,4,5-tetrahydro-3-benzazepin-1-ol, diastereomer 2 (1.086 g, 2.5278 mmol) in anhydrous methanol (50 mL) was added Mg (1.54 g, 63.361 mmol) powder (50 mesh). The reaction mixture was sonicated for 30 minutes. The unreacted Mg was filtered off through a pad of Celite and washed with methanol (10 mL). Saturated ammonium chloride aqueous solution (50 mL) was added to the filtrate, and it was extracted with dichloromethane (3×50 mL). The combined organic layers were dried over anhydrous sodium sulfate and concentrated under reduced pressure. The residue was purified by silica gel chromatography using 0 to 10% methanol in DCM (buffered with 0.2% ammonium hydroxide) to furnish (2R)-7-tert-butyl-2-isobutyl-2,3,4,5-tetrahydro-1H-3-benzazepin-1-ol, diastereomer 2 (502 mg, 70%) as a white solid. ¹H NMR (500 MHz, Chloroform-d) δ 7.18 (dd, J=7.8, 1.8 Hz, 1H), 7.14-7.09 (m, 2H), 4.39 (s, 1H), 3.41-3.30 (m, 2H), 2.91 (t, J=6.9 Hz, 1H), 2.87 (t, J=12.1 Hz, 1H), 2.70 (dd, J=15.2, 5.4 Hz, 1H), 1.73 (dq, J=13.4, 6.7 Hz, 1H), 1.57 (dt, J=14.0, 7.0 Hz, 1H), 1.43 (dt, J=13.9, 7.1 Hz, 1H), 1.32 (s, 9H), 0.94 (dd, J=6.5, 4.6 Hz, 6H). ESI-MS m/z calc. 275.2249, found 276.3 (M+1)⁺; Retention time: 1.81 minutes; LC method W.

### Step 14: (25R)-20-tert-Butyl-12-(2,6-dimethylphenyl)-25-(2-methylpropyl)-15-oxa-8λ⁶-thia-1,9,11,26-tetraazapentacyclo[14.8.1.13,7.110,14.017,22]heptacosa-3(27),4,6,10(26),11,13,17(22),18,20-nonaene-2,8,8-trione (Compound 237)

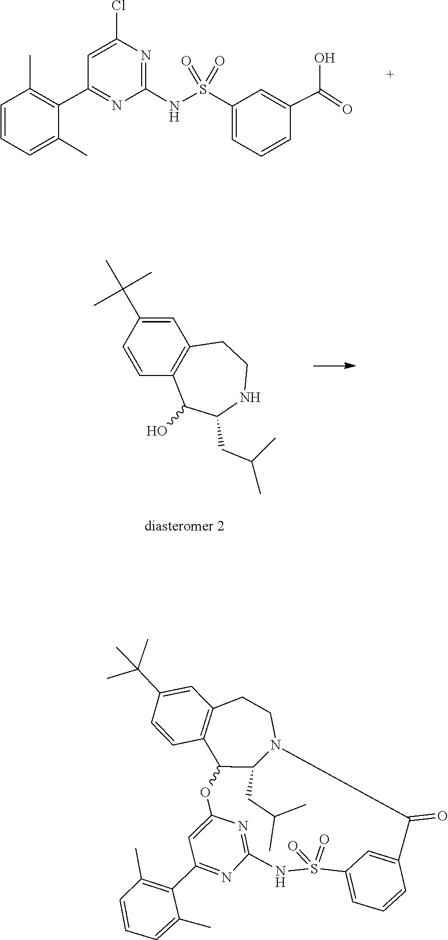

In a 3-mL vial, 3-[[4-chloro-6-(2,6-dimethylphenyl)pyrimidin-2-yl]sulfamoyl]benzoic acid (42.0 mg, 0.09579 mmol), (2R)-7-tert-butyl-2-isobutyl-2,3,4,5-tetrahydro-1H-3-benzazepin-1-ol, diastereomer 2 (28.0 mg, 0.09841 mmol) and NaOtBu (60 mg, 0.6243 mmol) were dissolved in THE (1.0 mL). The resulting mixture was stirred at room temperature for 1 h. In a separate 20-mL vial, a solution of HATU (90 mg, 0.2367 mmol) in DMF (4.0 mL) was prepared and cooled to 0° C. Then, the reaction mixture in the 3-mL vial was added dropwise (over 2 min) into this 20-mL vial. The resulting solution was stirred at 0° C. for 5 min, after which it was quenched by adding 1 N HCl solution (5 mL). The mixture was extracted with ethyl acetate (3×5 mL). The combined organic extracts were washed with water (10 mL) and saturated aqueous sodium chloride solution (10 mL), then dried over sodium sulfate, filtered, and evaporated in vacuo to give 130 mg of a yellow oil. Purification by silica gel chromatography (4 g of silica) using a gradient eluent of 1 to 80% ethyl acetate in hexanes gave 35 mg of a white solid that was not entirely pure. Further purification by reverse phase preparative chromatography using a C₁₈ column and a gradient eluent of 1 to 99% acetonitrile in water containing 5 mM hydrochloric acid gave a white solid, (25R)-20-tert-butyl-12-(2,6-dimethylphenyl)-25-(2-methylpropyl)-15-oxa-8λ⁶-thia-1,9,11,26-tetraazapentacyclo[14.8.1.13,7.110,14.017,22]heptacosa-3(27),4,6,10(26),11,13,17(22),18,20-nonaene-2,8,8-trione (11.1 mg, 18%)¹H NMR (500 MHz, DMSO-d₆) δ 13.59-12.02 (broad d, 1H), 8.61 (s, 1H), 7.90 (s, 1H), 7.68 (s, 2H), 7.66 (s, 1H), 7.38-7.26 (m, 3H), 7.15 (d, J=7.7 Hz, 2H), 6.63 (s, 1H), 6.50 (d, J=3.4 Hz, 1H), 4.81 (dd, J=13.8, 5.7 Hz, 1H), 4.07 (d, J=9.7 Hz, 1H), 3.15 (t, J=13.1 Hz, 1H), 3.07 (dd, J=15.2, 5.9 Hz, 1H), 2.79 (dd, J=13.5, 11.8 Hz, 1H), 2.27-1.89 (m, 6H), 1.37-1.22 (m, 2H), 1.31 (s, 9H), 1.03-0.93 (m, 1H), 0.69 (d, J=6.6 Hz, 3H), 0.28 (d, J=6.3 Hz, 3H). ESI-MS m/z calc. 638.29266, found 639.5 (M+1)⁺; Retention time: 2.31 minutes; LC method A.

### Example 134: Preparation of Compound 238

### Step 1: 4-Isoquinolylmethanol

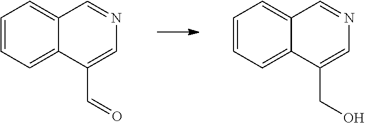 [SMILES: O=Cc1cncc2ccccc12 ; OCc1cncc2ccccc12]

To a solution of isoquinoline-4-carbaldehyde (5 g, 31.813 mmol) in ethanol (150 mL) at 0° C. was added sodium borohydride (1.35 g, 35.684 mmol). The mixture was stirred at 0° C. for 1.5 hours and at room temperature for 1.0 h. The reaction was quenched with a aqueous solution of ammonium acetate 25% (200 mL). Ethanol was removed under reduced pressure and the resultant mixture was extracted with ethyl acetate (3×100 mL). The combined organic extracts were washed with brine (100 mL), dried over sodium sulfate, filtered and concentrated under reduced pressure. The crude was evaporated 3 times with MeOH (50 mL) to remove residual ethyl acetate and dried under high vacuum to give 4-isoquinolylmethanol (4.81 g, 95%) as a brown oil. ¹H NMR (300 MHz, CDCl₃) δ 9.19 (s, 1H), 8.48 (s, 1H), 8.17 (d, J=8.2 Hz, 1H), 8.00 (d, J=8.2 Hz, 1H), 7.78 (ddd, J=8.2, 7.0, 1.2 Hz, 1H), 7.70-7.58 (m, 1H), 5.19-5.05 (m, 2H), 2.97 (br. s., 1H). ESI-MS m/z calc. 159.06842, found 160.1 (M+1)⁺; Retention time: 0.42 minutes; LC method K.

### Step 2: 1,2,3,4-Tetrahydroisoquinolin-4-ylmethanol

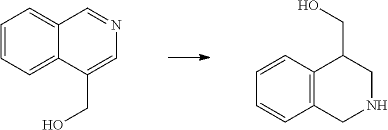 [SMILES: OCc1cncc2ccccc12 ; OCC1CNCc2ccccc21]

4-Isoquinolylmethanol (4.81 g, 30.216 mmol) was mixed with platinum dioxide (700 mg, 3.0826 mmol) and hydrochloric acid (32 mL of 1 M, 32.000 mmol) under nitrogen atmosphere in solution in methanol (500 mL). The solution was shaken for 4 hours at room temperature under hydrogen atmosphere at 50 psi in a Parr apparatus. The reaction mixture was filtered, and the filtrate was concentrated to dryness by rotary evaporation then high vacuum overnight. The crude mixture (5.7 g) was diluted in water (16 mL) and divided in four parts. Each part was purified by reverse phase chromatography. The fractions were combined and concentrated by rotary evaporation. The residue was lyophilized to give 1,2,3,4-tetrahydroisoquinolin-4-ylmethanol (hydrochloride salt) (855 mg, 14%) as a white solid. ¹H NMR (300 MHz, DMSO-d₆) δ 9.74 (br. s., 1H), 9.07 (br. s., 1H), 7.43-7.17 (m, 4H), 4.22 (t, J=4.3 Hz, 2H), 3.79 (dd, J=11.7, 5.0 Hz, 1H), 3.67 (dd, J=10.7, 7.8 Hz, 1H), 3.44-3.35 (m, 2H), 3.20-3.07 (m, 1H). ESI-MS m/z calc. 163.09972, found 164.2 (M+1)⁺; Retention time: 0.42 minutes; LC method U.

### Step 3: 12-(2,6-Dimethylphenyl)-15-oxa-8λ⁶-thia-1,9,11,26-tetraazapentacyclo[15.7.1.13,7.110,14.018,23]heptacosa-3,5,7(27),10(26),11,13,18,20,22-nonaene-2,8,8-trione (Compound 238)

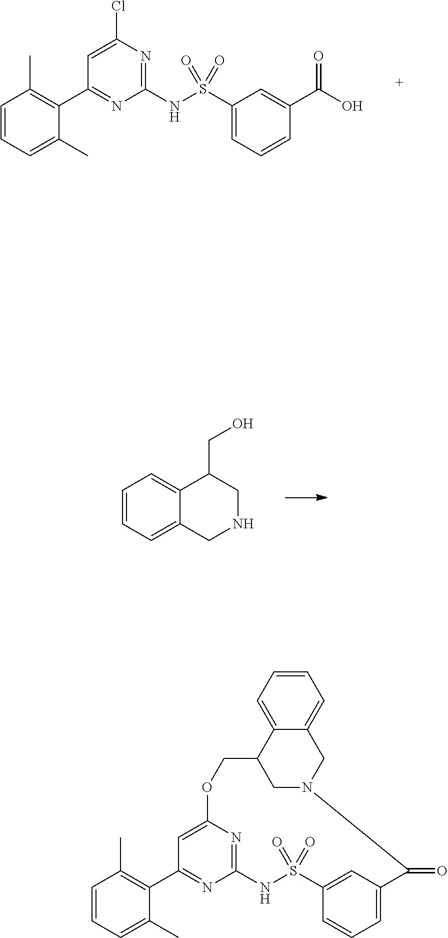 [SMILES: Cc1cccc(C)c1-c1cc(Cl)nc(NS(=O)(=O)c2cccc(C(=O)O)c2)n1 ; OCC1CNCc2ccccc21 ; Cc1cccc(C)c1-c1cc2nc(n1)NS(=O)(=O)c1cccc(c1)C(=O)N1Cc3ccccc3C(CO2)C1]

To a solution of 3-[[4-chloro-6-(2,6-dimethylphenyl)pyrimidin-2-yl]sulfamoyl]benzoic acid (114 mg, 0.2728 mmol) and 1,2,3,4-tetrahydroisoquinolin-4-ylmethanol (hydrochloride salt) (63 mg, 0.3155 mmol) in THE (1 mL) was added sodium tert-butoxide (135 mg, 1.405 mmol) and the reaction mixture was stirred at room temperature for 2 hours. The reaction mixture was then added dropwise to a solution of HATU (215 mg, 0.5654 mmol) in DMF (1 mL). To this solution was added DiPEA (143 μL, 0.8210 mmol) and stirred at room temperature for 16 hours. The solvent was evaporated then taken up in 1:1 DMSO MeOH, filtered and purification by HPLC (1-99% ACN in water (HCl modifier)) gave 12-(2,6-dimethylphenyl)-15-oxa-8λ⁶-thia-1,9,11,26-tetraazapentacyclo[15.7.1.13,7.110,14.018,23]heptacosa-3,5,7(27),10(26),11,13,18,20,22-nonaene-2,8,8-trione (7.9 mg, 5%). ESI-MS m/z calc. 526.1675, found 527.3 (M+1)⁺; Retention time: 1.58 minutes; LC method A.

### Example 135: Preparation of Compound 239 and Compound 240

### Step 1: Methyl 3-[(3-methoxycarbonylphenyl)disulfanyl]benzoate

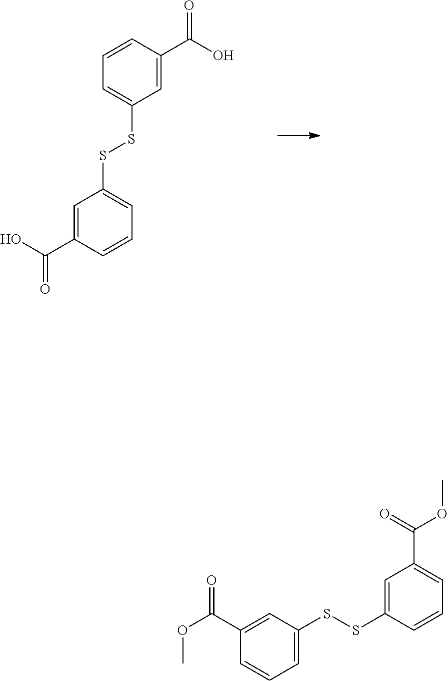 [SMILES: O=C(O)c1cccc(SSc2cccc(C(=O)O)c2)c1 ; COC(=O)c1cccc(SSc2cccc(C(=O)OC)c2)c1]

A suspension of 3-[(3-carboxyphenyl)disulfanyl]benzoic acid (4.49 g, 14.7 mmol) in methanol (140 mL) and sulfuric acid (2.6 mL, 47 mmol) was refluxed for 17 hours. Once cooled, the crude mixture (now a solution) was concentrated under reduced pressure to remove most of the methanol. The residue was then transferred to a 1.0-L separatory funnel with 5% aqueous sodium bicarbonate (250 mL) and the aqueous layer was extracted with ethyl acetate (2×200 mL). The combined organic layers were washed with brine (100 mL), dried over sodium sulfate, filtered and concentrated under reduced pressure to afford crude methyl 3-[(3-methoxycarbonylphenyl)disulfanyl]benzoate (4.915 g, 97.0% purity, 97% yield) as an amber oil. ESI-MS m/z calc. 334.03336, found 335.1 (M+1)⁺; Retention time: 2.38 minutes; LC method C.

### Step 2: Methyl 3-[[4-chloro-6-(2,6-dimethylphenyl)pyrimidin-2-yl]amino]sulfanylbenzoate

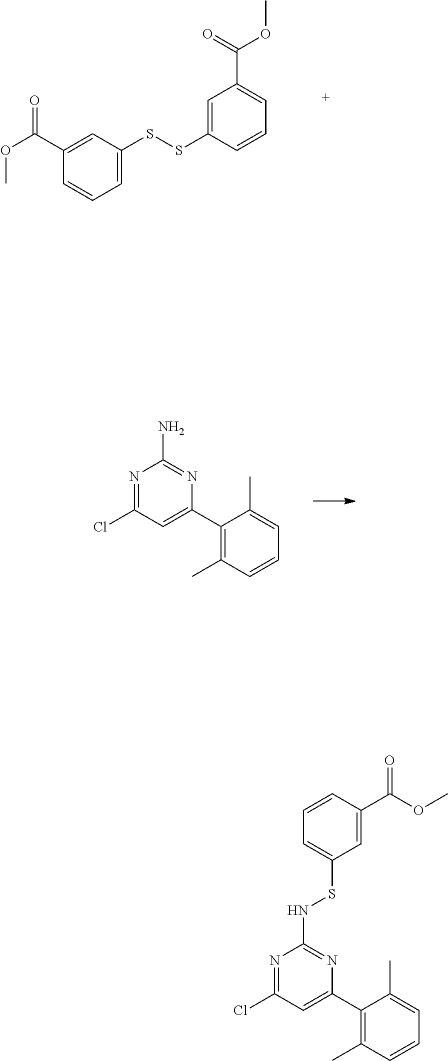 [SMILES: COC(=O)c1cccc(SSc2cccc(C(=O)OC)c2)c1 ; Cc1cccc(C)c1-c1cc(Cl)nc(N)n1 ; COC(=O)c1cccc(SNc2nc(Cl)cc(-c3c(C)cccc3C)n2)c1]

A solution of methyl 3-[(3-methoxycarbonylphenyl)disulfanyl]benzoate (3.983 g, 11.91 mmol) in dichloromethane (25 mL) containing nine drops of pyridine was treated with sulfuryl chloride (0.96 mL, 11.8 mmol) and stirred at room temperature for 10 minutes (orange color appears). In a separate flask, 4-chloro-6-(2,6-dimethylphenyl)pyrimidin-2-amine hydrochloride (4.60 g, 17.0 mmol) was suspended in dichloromethane (95 mL) and triethylamine (9.7 mL, 70 mmol) was added (produced a solution). This solution was cooled in an ice-bath and the solution containing the sulfenyl chloride was added dropwise over about 10 minutes. The flask was kept in the ice-bath and left to gradually warm to room temperature for 5 hours. The crude mixture was transferred to a 500-mL separatory funnel with 5% sodium bicarbonate (250 mL) and brine (25 mL) and the aqueous layer was extracted with dichloromethane (2×100 mL). The combined organic layers were dried over sodium sulfate, filtered and concentrated under reduced pressure. The residue was purified by silica gel chromatography on a 220-g column, eluting from 0% to 30% ethyl acetate in heptanes. Pure fractions were set aside, and the mixed fractions were purified a second time by silica gel chromatography on a 220-g column, eluting from 0% to 30% ethyl acetate in heptanes. Both set of pure fractions were combined together to afford methyl 3-[[4-chloro-6-(2,6-dimethylphenyl)pyrimidin-2-yl]amino]sulfanylbenzoate (3.591 g, 98.1% purity, 52%) as a white solid. ESI-MS m/z calc. 399.0808, found 400.1 (M+1)⁺; Retention time: 2.37 minutes; LC method C.

### Step 3: Methyl 3-[4-chloro-6-(2,6-dimethylphenyl)pyrimidin-2-yl]sulfinamoylbenzoate

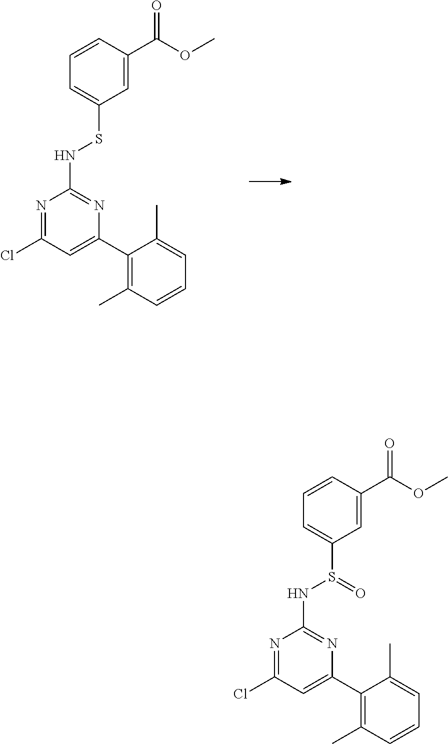 [SMILES: COC(=O)c1cccc(SNc2nc(Cl)cc(-c3c(C)cccc3C)n2)c1 ; COC(=O)c1cccc(S(=O)Nc2nc(Cl)cc(-c3c(C)cccc3C)n2)c1]

A solution of methyl 3-[[4-chloro-6-(2,6-dimethylphenyl)pyrimidin-2-yl]amino]sulfanylbenzoate (4.293 g, 10.74 mmol) in dichloromethane (100 mL) was cooled in an ice-bath and treated with m-CPBA (2.57 g, 77% purity, 11.5 mmol) and the reaction was left to stir in the ice-bath for 1 hour. The reaction was then quenched with 10% sodium thiosulfate (30 mL) and the crude mixture was transferred to a 500-mL separatory funnel with 5% aqueous sodium bicarbonate (200 mL) and extracted with dichloromethane (2×100 mL). The combined organic layers were dried over sodium sulfate, filtered and concentrated under reduced pressure. The residue was purified by silica gel chromatography on a 220-g column, eluting from 0% to 40% ethyl acetate in heptanes, to afford methyl 3-[4-chloro-6-(2,6-dimethylphenyl)pyrimidin-2-yl]sulfinamoylbenzoate (2.49 g, 99.2% purity, 55%) as a white solid. ESI-MS m/z calc. 415.0757, found 416.1 (M+1)⁺; Retention time: 2.22 minutes (LC method C). Additional fractions containing product were concentrated under reduced pressure and triturated, triturated in ethyl acetate (about 20 mL), filtered and concentrated under reduced pressure to afford a second lot of methyl 3-[4-chloro-6-(2,6-dimethylphenyl)pyrimidin-2-yl]sulfinamoylbenzoate (0.962 g, 99.3% purity, 21%) as a white solid. ESI-MS m/z calc. 415.0757, found 416.2 (M+1)⁺; Retention time: 2.20 minutes (LC method C).

### Step 4: Methyl 3-[[[4-chloro-6-(2,6-dimethylphenyl)pyrimidin-2-yl]amino]sulfonimidoyl]benzoate

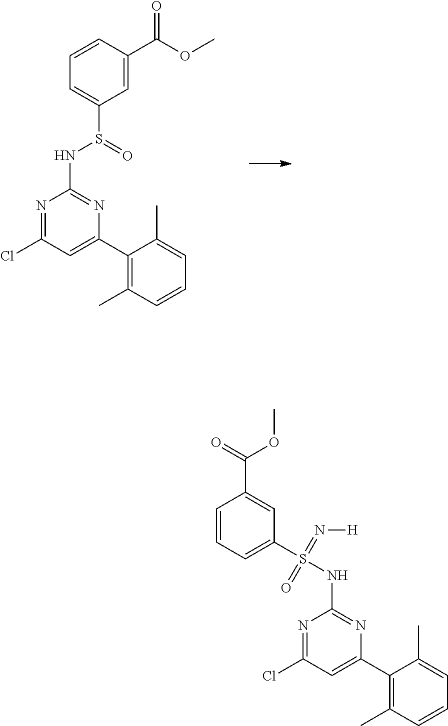 [SMILES: COC(=O)c1cccc(S(=O)Nc2nc(Cl)cc(-c3c(C)cccc3C)n2)c1 ; COC(=O)c1cccc(S(=N)(=O)Nc2nc(Cl)cc(-c3c(C)cccc3C)n2)c1]

A solution of methyl 3-[4-chloro-6-(2,6-dimethylphenyl)pyrimidin-2-yl]sulfinamoylbenzoate (2.29 g, 5.51 mmol) in dichloromethane (85 mL) at room temperature was treated with 1-chloropyrrolidine-2,5-dione (1.03 g, 7.71 mmol) and the solution was stirred at room temperature for 7 hours. The solution was then cooled in an ice-bath and treated with a solution of ammonia (100 mL of 0.5 M in dioxane, 50 mmol) over a period of 10-15 minutes using an addition funnel. After the addition, the reaction was removed from the ice-bath and stirred for 16 hours at room temperature. The crude mixture was transferred to a 1.0-L separatory funnel with 5% aqueous sodium bicarbonate (600 mL) and brine (100 mL) and the aqueous layer was extracted with dichloromethane (200 mL+2×150 mL). The combined organic layers were washed with brine (150 mL), dried over sodium sulfate, filtered and concentrated under reduced pressure. The residue was purified by silica gel chromatography on a 120-g column, eluting from 0% to 50% ethyl acetate in heptanes, to afford methyl 3-[[[4-chloro-6-(2,6-dimethylphenyl)pyrimidin-2-yl]amino]sulfonimidoyl]benzoate (2.21 g, 93%) as a white solid. ESI-MS m/z calc. 430.08664, found 431.1 (M+1)⁺; Retention time: 2.01 minutes; LC method C.

### Step 5: 3-[[[4-Chloro-6-(2,6-dimethylphenyl)pyrimidin-2-yl]amino]sulfonimidoyl]benzoic acid

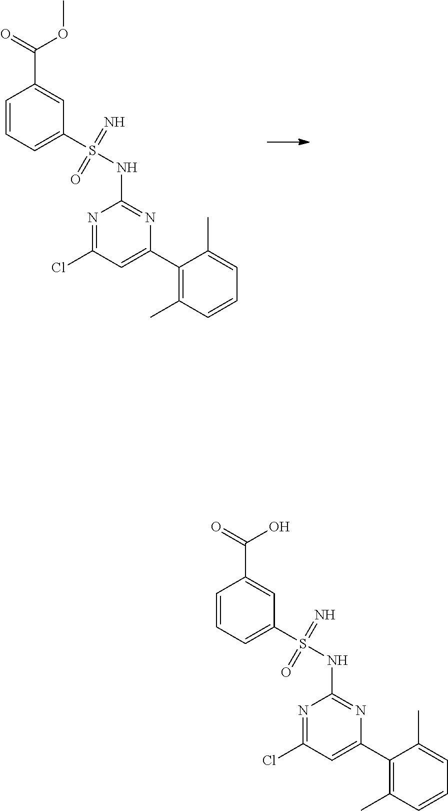 [SMILES: COC(=O)c1cccc(S(=N)(=O)Nc2nc(Cl)cc(-c3c(C)cccc3C)n2)c1 ; Cc1cccc(C)c1-c1cc(Cl)nc(NS(=N)(=O)c2cccc(C(=O)O)c2)n1]

A mixture of methyl 3-[[[4-chloro-6-(2,6-dimethylphenyl)pyrimidin-2-yl]amino]sulfonimidoyl]benzoate (2.57 g, 5.96 mmol) in THE (65 mL) and water (65 mL) was treated with lithium hydroxide monohydrate (500 mg, 11.9 mmol) and the reaction was stirred vigorously at room temperature for 4.5 hours. Another portion of lithium hydroxide monohydrate (250 mg, 5.96 mmol) was added and stirring was continued for another 2 hours. Most of the THF was removed under reduced pressure, and the aqueous layer was acidified to a pH of about 4 with solid citric acid. The reaction mixture was transferred to a 500-mL separatory funnel and the aqueous layer was extracted with ethyl acetate (3×100 mL). The combined organic layers were washed with water (2×50 mL), brine (50 mL), dried over sodium sulfate, filtered and concentrated under reduced pressure to afford 3-[[[4-chloro-6-(2,6-dimethylphenyl)pyrimidin-2-yl]amino]sulfonimidoyl]benzoic acid (2.493 g, 95.4% purity, 96%) as an off-white solid. ¹H NMR (300 MHz, DMSO-d₆) δ 1.75 (br. s., 6H), 6.91 (s, 1H), 7.03 (d, J=7.6 Hz, 2H), 7.12-7.22 (m, 1H), 7.64 (t, J=7.9 Hz, 1H), 7.84 (s, 2H), 8.06 (d, J=7.9 Hz, 1H), 8.11 (d, J=7.6 Hz, 1H), 8.39 (t, J=1.6 Hz, 1H), 13.35 (br. s., 1H). ESI-MS m/z calc. 416.07098, found 417.1 (M+1)⁺; Retention time: 2.59 minutes (LC method B).

### Step 6: tert-Butyl 4-[3-[[[4-chloro-6-(2,6-dimethylphenyl)pyrimidin-2-yl]amino]sulfonimidoyl]benzoyl]-6-hydroxy-1,4-diazepane-1-carboxylate

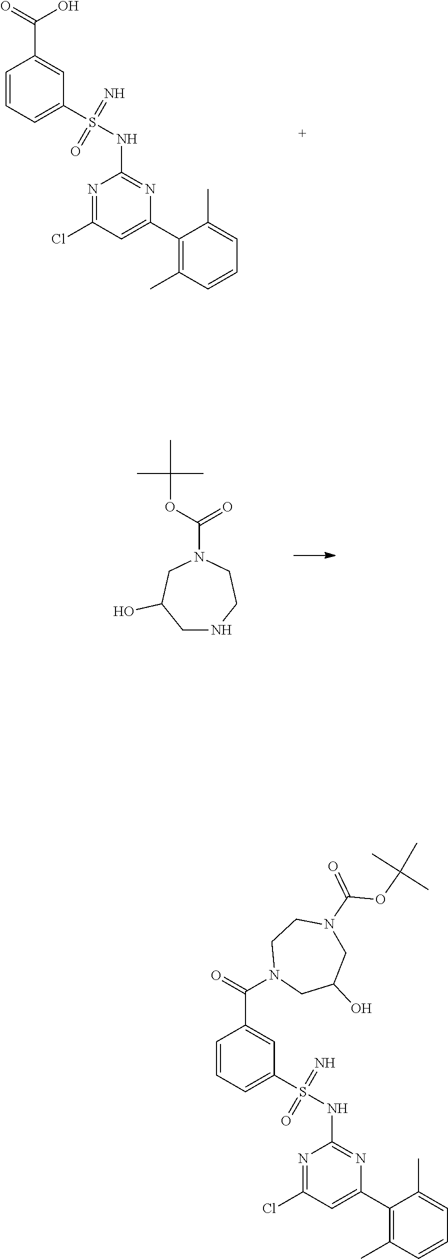 [SMILES: Cc1cccc(C)c1-c1cc(Cl)nc(NS(=N)(=O)c2cccc(C(=O)O)c2)n1 ; CC(C)(C)OC(=O)N1CCNCC(O)C1 ; Cc1cccc(C)c1-c1cc(Cl)nc(NS(=N)(=O)c2cccc(C(=O)N3CCN(C(=O)OC(C)(C)C)CC(O)C3)c2)n1]

To a solution of 3-[[[4-chloro-6-(2,6-dimethylphenyl)pyrimidin-2-yl]amino]sulfonimidoyl]benzoic acid (2.13 g, 4.87 mmol) and 6-hydroxy-[1,4]diazepane-1-carboxylic acid tert-butyl ester (1.32 g, 5.80 mmol) in dimethylformamide (40 mL) and DIEA (10 mL, 57.41 mmol) was added HATU (1.94 g, 5.10 mmol) at 0° C. The reaction mixture was stirred for 10 minutes in an ice-salt bath. Then reaction was quenched with 10% aqueous citric acid solution (100 mL). This aqueous phase was extracted with ethyl acetate (3×100 mL) and the combined organic layers were washed with brine (200 mL), dried over anhydrous sodium sulfate and concentrated. The residue was purified by silica gel column chromatography using 0-80% hexanes-acetone to furnish tert-butyl 4-[3-[[[4-chloro-6-(2,6-dimethylphenyl)pyrimidin-2-yl]amino]sulfonimidoyl]benzoyl]-6-hydroxy-1,4-diazepane-1-carboxylate (2.4 g, 78%). ESI-MS m/z calc. 614.2078, found 615.5 (M+1)⁺; Retention time: 3.02 minutes; (LC method T).

### Step 7: tert-Butyl 12-(2,6-dimethylphenyl)-8-imino-2,8-dioxo-15-oxa-8λ⁶-thia-1,9,11,18,22-pentaazatetracyclo[14.4.1.13,7.110,14]tricosa-3,5,7(23),10,12,14(22)-hexaene-18-carboxylate, diastereomeric pair 1 and 2

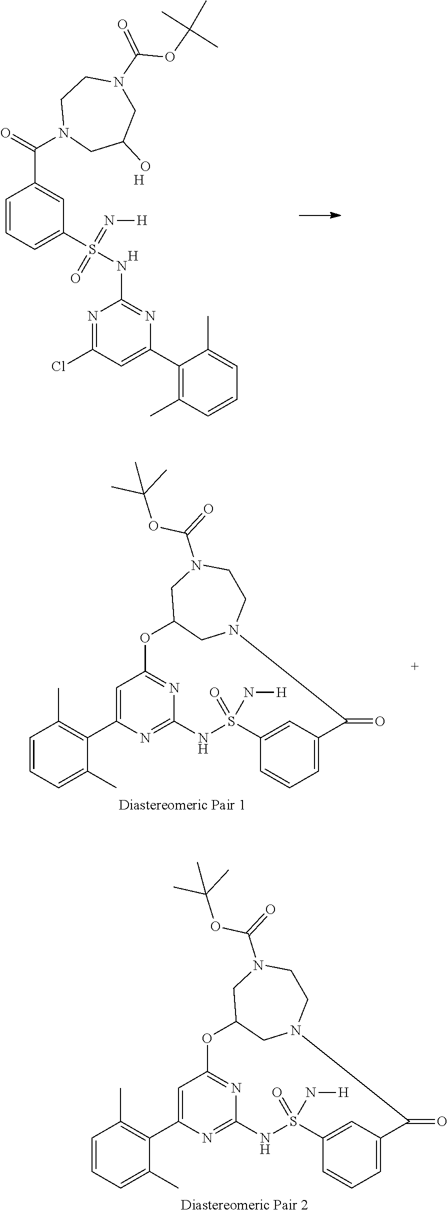

To a solution of tert-butyl 4-[3-[[[4-chloro-6-(2,6-dimethylphenyl)pyrimidin-2-yl]amino]sulfonimidoyl]benzoyl]-6-hydroxy-1,4-diazepane-1-carboxylate (2.4 g, 3.82 mmol) in anhydrous dimethylformamide (250 mL) was added 60% suspension sodium hydride in mineral oil (1.4 g, 35.0 mmol) in two portions. The reaction mixture was stirred at 0° C. to room temperature for 1.5 hours and then quenched with 10% aqueous citric acid solution (200 mL). The reaction mixture was extracted with ethyl acetate (3×200 mL) and the combined organic layers were washed with brine (3×500 mL), dried over anhydrous sodium sulfate and concentrated. The residue was purified by silica gel column chromatography using 0-70% hexanes-acetone to afford two partially separated isomers of tert-butyl 16-(2,6-dimethylphenyl)-4-oxo-2-oxa-6-sulfonimidoyl-7-aza-3(6,1)-diazepana-1(4,2)-pyrimidina-5(1,3)-benzenacycloheptaphane-34-carboxylate: Diastereomeric pair 1, tert-butyl 12-(2,6-dimethylphenyl)-8-imino-2,8-dioxo-15-oxa-8λ⁶-thia-1,9,11,18,22-pentaazatetracyclo[14.4.1.13,7.110,14]tricosa-3,5,7(23),10,12,14(22)-hexaene-18-carboxylate (532 mg, 23%), ESI-MS m/z calc. 578.23, found 579.4 (M+1)⁺; Retention time: 1.86 minutes, (LC method W); ¹H NMR (250 MHz, DMSO-d₆) δ 8.47 (s, 1H), 8.05 (d, J=25.0 Hz, 3H), 7.69 (s, 2H), 7.11 (d, J=20.3 Hz, 3H), 6.15 (s, 1H), 5.45 (s, 1H), 4.44 (s, 1H), 4.30-3.71 (m, 2H), 3.56 (s, 2H), 3.22 (s, 3H), 1.97 (s, 6H), 1.59-1.12 (m, 10H); and diastereomeric pair 2: tert-butyl 12-(2,6-dimethylphenyl)-8-imino-2,8-dioxo-15-oxa-8λ⁶-thia-1,9,11,18,22-pentaazatetracyclo[14.4.1.13,7.110,14]tricosa-3,5,7(23),10,12,14(22)-hexaene-18-carboxylate (727 mg, 31%) ESI-MS m/z calc. 578.23, found 579.4 (M+1)⁺; Retention time: 1.92 minutes (LC method W), ¹H NMR (250 MHz, DMSO-d₆) δ 8.30 (d, J=16.0 Hz, 1H), 8.08 (d, J=8.1 Hz, 3H), 7.79-7.57 (m, 2H), 7.13 (d, J=23.0 Hz, 3H), 5.63-5.23 (m, 1H), 4.71-4.31 (m, 1H), 4.23-3.76 (m, 3H), 3.28-3.00 (m, 5H), 1.98 (s, 8H), 1.45 (d, J=10.6 Hz, 10H).

### Step 8: tert-Butyl 12-(2,6-dimethylphenyl)-8-imino-2,8-dioxo-15-oxa-8λ⁶-thia-1,9,11,18,22-pentaazatetracyclo[14.4.1.13,7.110,14]tricosa-3,5,7(23),10,12,14(22)-hexaene-18-carboxylate (Diastereomeric Pair 1) (Compound 239), and tert-butyl 12-(2,6-dimethylphenyl)-8-imino-2,8-dioxo-15-oxa-8λ⁶-thia-1,9,11,18,22-pentaazatetracyclo[14.4.1.13,7.110,14]tricosa-3,5,7(23),10,12,14(22)-hexaene-18-carboxylate, (Diastereomeric Pair 2) (Compound 240)

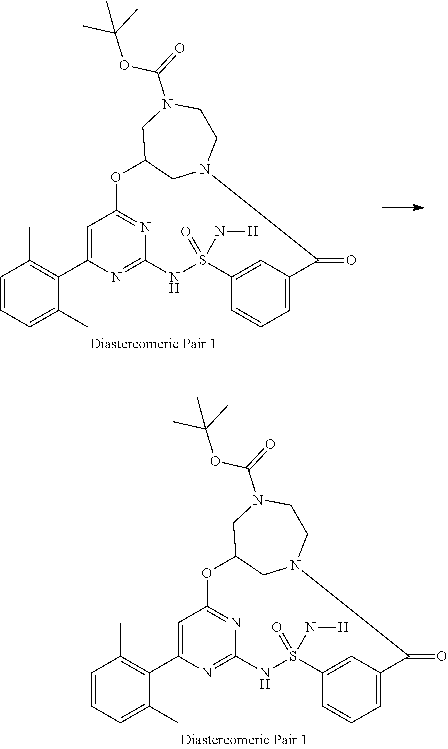

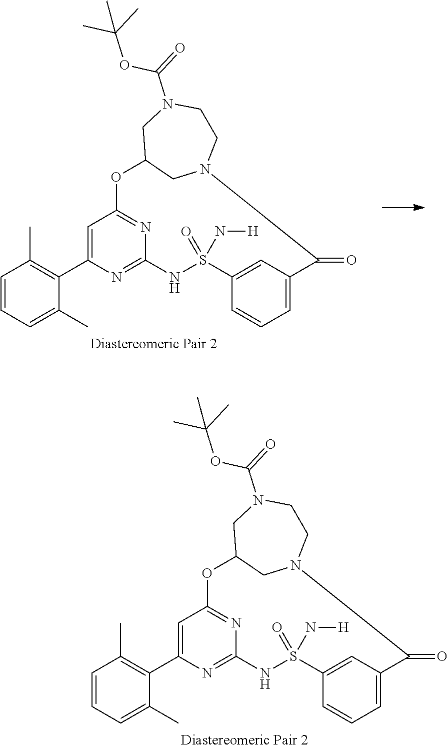

Partially separated tert-butyl 12-(2,6-dimethylphenyl)-8-imino-2,8-dioxo-15-oxa-8λ⁶-thia-1,9,11,18,22-pentaazatetracyclo[14.4.1.13,7.110,14]tricosa-3,5,7(23),10,12,14(22)-hexaene-18-carboxylate (532 mg, 0.9193 mmol)(Diastereomer 1) and tert-butyl 12-(2,6-dimethylphenyl)-8-imino-2,8-dioxo-15-oxa-8λ⁶-thia-1,9,11,18,22-pentaazatetracyclo[14.4.1.13,7.110,14]tricosa-3,5,7(23),10,12,14(22)-hexaene-18-carboxylate (727 mg, 1.256 mmol) (Diastereomer 2) were purified by reverse phase preparative chromatography using a C₁₈ column and a 15 min. gradient eluent of 10 to 400% acetonitrile in water containing 5 mM hydrochloric acid to give: First diastereomeric pair (came out earlier on HPLC)—Diastereomeric Pair, tert-butyl 12-(2,6-dimethylphenyl)-8-imino-2,8-dioxo-15-oxa-8λ⁶-thia-1,9,11,18,22-pentaazatetracyclo[14.4.1.13,7.110,14]tricosa-3,5,7(23),10,12,14(22)-hexaene-18-carboxylate (383 mg, 720%), ¹H NMR (400 MHz, DMSO-d₆) δ 8.83 (s, 1H), 8.48 (d, J=26.7 Hz, 1H), 8.06 (s, 1H), 7.76 (s, 2H), 7.19 (d, J=49.5 Hz, 3H), 6.57 (s, 1H), 5.43 (s, 1H), 4.46 (s, 1H), 4.12 (dd, J=49.4, 15.5 Hz, 1H), 3.89 (d, J=25.7 Hz, 1H), 3.66 (s, 1H), 3.20 (s, 2H), 2.04 (s, 6H), 1.43 (s, 9H). ESI-MS m/z calc. 578.23114, found 579.0 (M+1)⁺; Retention time: 1.18 minutes (LC method A); and second diastereomeric pair (came out later on HPLC)—Diastereomeric Pair 2, tert-butyl 12-(2,6-dimethylphenyl)-8-imino-2,8-dioxo-15-oxa-8λ⁶-thia-1,9,11,18,22-pentaazatetracyclo[14.4.1.13,7.110,14]tricosa-3,5,7(23),10,12,14(22)-hexaene-18-carboxylate (90 mg, 17%)¹H NMR (400 MHz, DMSO-d₆) δ 8.23 (d, J=25.1 Hz, 1H), 8.06 (s, 1H), 7.67 (d, J 26.8 Hz, 2H), 7.11 (d, J=48.3 Hz, 3H), 6.27 (s, 1H), 5.42 (s, 1H), 4.41 (s, 1H), 3.99 (d, J=48.4 Hz, 3H), 3.07 (s, 2H), 1.94 (s, 6H), 1.37 (d, J=17.6 Hz, 9H). ESI-MS m/z calc. 578.23114, found 579.0 (M+1)⁺; Retention time: 1.27 minutes (LC method A).

### Example 136: Preparation of Compound 241

### Step 1: 12-(2,6-Dimethylphenyl)-8-imino-15-oxa-8λ⁶-thia-1,9,11,18,22-pentaazatetracyclo[14.4.1.13,7.110,14]tricosa-3,5,7(23),10,12,14(22)-hexaene-2,8-dione, diastereomeric pair 1 (Compound 241)

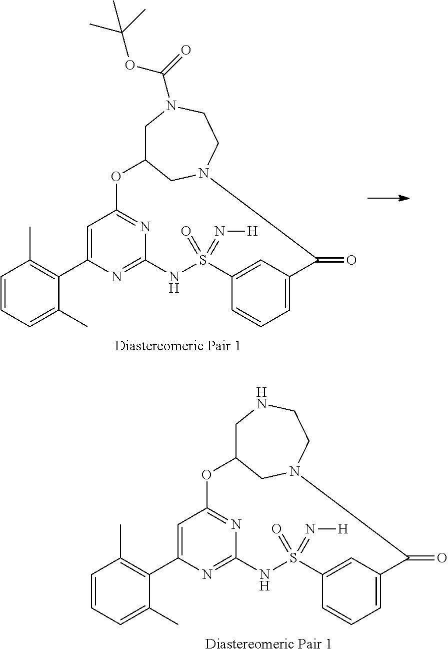 [SMILES: Cc1cccc(C)c1-c1cc2nc(n1)NS(=N)(=O)c1cccc(c1)C(=O)N1CCN(C(=O)OC(C)(C)C)CC(C1)O2 ; Cc1cccc(C)c1-c1cc2nc(n1)NS(=N)(=O)c1cccc(c1)C(=O)N1CCNCC(C1)O2]

tert-Butyl 12-(2,6-dimethylphenyl)-8-imino-2,8-dioxo-15-oxa-8λ⁶-thia-1,9,11,18,22-pentaazatetracyclo[14.4.1.13,7.110,14]tricosa-3,5,7(23),10,12,14(22)-hexaene-18-carboxylate (383 mg, 0.6585 mmol) (Diastereomeric Pair 1) in HCl (5 mL of 4 M, 20.00 mmol) was stirred for 1 h. Volatiles were removed and the crude was azeotroped with toluene three times. A small amount was purified by reverse phase preparative chromatography using a C₁₈ column and a 15 min. gradient eluent of 1 to 50% acetonitrile in water containing 5 mM hydrochloric acid to give 12-(2,6-dimethylphenyl)-8-imino-15-oxa-8λ⁶-thia-1,9,11,18,22-pentaazatetracyclo[14.4.1.13,7.110,14]tricosa-3,5,7(23),10,12,14(22)-hexaene-2,8-dione, diastereomeric pair 1(hydrochloride salt) (354 mg, 104%)¹H NMR (400 MHz, DMSO-d₆) δ 10.35 (s, 1H), 10.12 (s, 1H), 8.92 (s, 3H), 8.09 (d, J=10.8 Hz, 1H), 7.85-7.73 (m, 2H), 7.32 (t, J=7.6 Hz, 1H), 7.17 (d, J=7.5 Hz, 2H), 6.66 (s, 1H), 6.01 (s, 1H), 4.54 (s, 1H), 3.80 (d, J=17.7 Hz, 1H), 3.72 (d, J=8.1 Hz, 1H), 3.62 (d, J=14.7 Hz, 1H), 3.47 (d, J=9.2 Hz, 4H), 3.27 (d, J=14.3 Hz, 2H), 2.54 (s, 2H), 2.09 (s, 6H). ESI-MS m/z calc. 478.1787, found 478.0 (M+1)⁺; Retention time: 0.65 minutes (LC method A).

### Example 137: Preparation of Compound 242

### Step 1: 12-(2,6-Dimethylphenyl)-8-imino-15-oxa-8λ⁶-thia-1,9,11,18,22-pentaazatetracyclo[14.4.1.13,7.110,14]tricosa-3,5,7(23),10,12,14(22)-hexaene-2,8-dione (Compound 242)

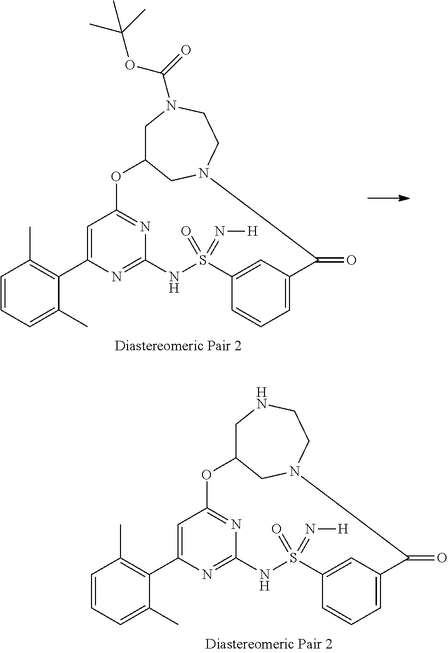 [SMILES: Cc1cccc(C)c1-c1cc2nc(n1)NS(=N)(=O)c1cccc(c1)C(=O)N1CCN(C(=O)OC(C)(C)C)CC(C1)O2 ; Cc1cccc(C)c1-c1cc2nc(n1)NS(=N)(=O)c1cccc(c1)C(=O)N1CCNCC(C1)O2]

tert-Butyl 12-(2,6-dimethylphenyl)-8-imino-2,8-dioxo-15-oxa-8λ⁶-thia-1,9,11,18,22-pentaazatetracyclo[14.4.1.13,7.110,14]tricosa-3,5,7(23),10,12,14(22)-hexaene-18-carboxylate (10 mg, 0.01719 mmol) (diastereomeric pair 2) in HCl (500 μL of 4 M, 2.000 mmol) was stirred for 1 h. Solvents were removed and the crude was filtered, and purified by reverse phase preparative chromatography using a C₁₈ column and a 15 min. gradient eluent of 1 to 50% acetonitrile in water containing 5 mM hydrochloric acid to give 12-(2,6-dimethylphenyl)-8-imino-15-oxa-8λ⁶-thia-1,9,11,18,22-pentaazatetracyclo[14.4.1.13,7.110,14]tricosa-3,5,7(23),10,12,14(22)-hexaene-2,8-dione, diastereomeric pair 2 (hydrochloride salt) (1.8 mg, 20%) ESI-MS m/z calc. 478.1787, found 479.0 (M+1)⁺; Retention time: 0.62 minutes; LC method A.

### Example 138: Preparation of Compound 243

### Step 1: 18-(3,3-Dimethylbutyl)-12-(2,6-dimethylphenyl)-8-imino-15-oxa-8λ⁶-thia-1,9,11,18,22-pentaazatetracyclo[14.4.1.13,7.110,14]tricosa-3,5,7(23),10,12,14(22)-hexaene-2,8-dione (Compound 243)

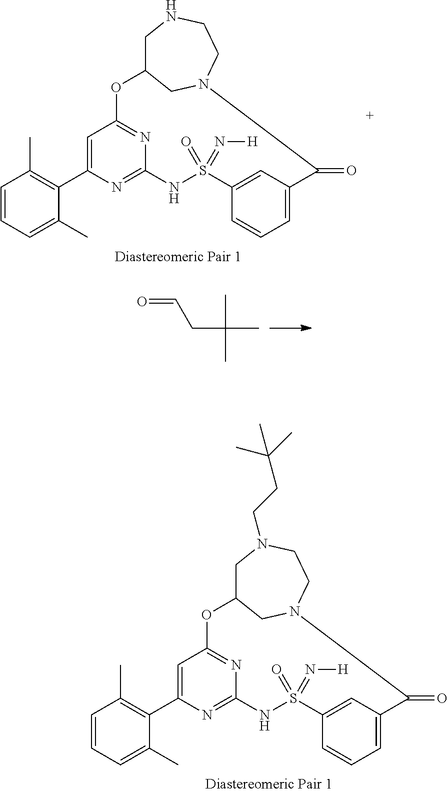

12-(2,6-Dimethylphenyl)-8-imino-15-oxa-8Δ⁶-thia-1,9,11,18,22-pentaazatetracyclo[14.4.1.13,7.110,14]tricosa-3,5,7(23),10,12,14(22)-hexaene-2,8-dione (hydrochloride salt) (54.5 mg, 0.1058 mmol) (Diastereomeric Pair 1) was combined with 3,3-dimethylbutanal (20 μL, 0.1593 mmol), acetic acid (30 μL, 0.5275 mmol), in DCE (2 mL), and stirred for 1 hour at room temperature. sodium cyanoborohydride (32 mg, 0.5092 mmol) was then added and the reaction was stirred for an additional hour at room temperature, then diluted with methanol, filtered, and purified by preparative HPLC (1-50% ACN in water, with HCl, 15 minute run) to give 18-(3,3-dimethylbutyl)-12-(2,6-dimethylphenyl)-8-imino-15-oxa-8λ⁶-thia-1,9,11,18,22-pentaazatetracyclo[14.4.1.13,7.110,14]tricosa-3,5,7(23),10,12,14(22)-hexaene-2,8-dione (26.1 mg, 44%) ¹H NMR (400 MHz, DMSO-d₆) δ 12.04 (s, 1H), 9.17 (s, 1H), 8.88 (s, 1H), 8.08 (d, J=7.2 Hz, 1H), 7.80 (s, 2H), 7.33 (t, J=7.6 Hz, 1H), 7.19 (s, 2H), 6.69 (s, 1H), 6.37 (s, 1H), 4.55 (d, J=19.1 Hz, 1H), 4.04 (s, 1H), 3.87 (d, J=17.2 Hz, 1H), 3.76 (d, J=13.6 Hz, 1H), 3.31 (s, 3H), 2.12 (s, 6H), 1.81 (s, 2H), 0.94 (s, 9H). ESI-MS m/z calc. 562.2726, found 563.0 (M+1)⁺; Retention time: 0.95 minutes (LC method A).

### Example 139: Preparation of Compound 244 and Compound 245

### Step 1: 18-(3,3-dimethylbutyl)-12-(2,6-dimethylphenyl)-8-imino-15-oxa-8λ⁶-thia-1,9,11,18,22-pentaazatetracyclo[14.4.1.13,7.110,14]tricosa-3,5,7(23),10,12,14(22)-hexaene-2,8-dione (Diastereomer 1a) (Compound 244), and 18-(3,3-dimethylbutyl)-12-(2,6-dimethylphenyl)-8-imino-15-oxa-8λ⁶-thia-1,9,11,18,22-pentaazatetracyclo[14.4.1.13,7.110,14]tricosa-3,5,7(23),10,12,14(22)-hexaene-2,8-dione (Diastereomer 1b) (Compound 245)

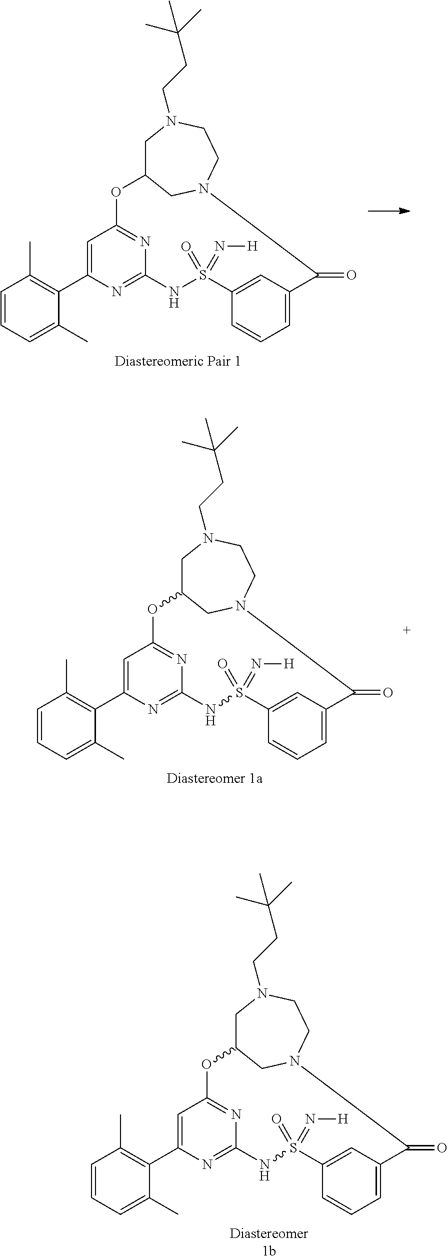 [SMILES: Cc1cccc(C)c1-c1cc2nc(n1)NS(=N)(=O)c1cccc(c1)C(=O)N1CCN(CCC(C)(C)C)CC(C1)O2 ; Cc1cccc(C)c1-c1cc2nc(n1)NS(=N)(=O)c1cccc(c1)C(=O)N1CCN(CCC(C)(C)C)CC(C1)O2 ; Cc1cccc(C)c1-c1cc2nc(n1)NS(=N)(=O)c1cccc(c1)C(=O)N1CCN(CCC(C)(C)C)CC(C1)O2]

18-(3,3-Dimethylbutyl)-12-(2,6-dimethylphenyl)-8-imino-15-oxa-8λ⁶-thia-1,9,11,18,22-pentaazatetracyclo[14.4.1.13,7.110,14]tricosa-3,5,7(23),10,12,14(22)-hexaene-2,8-dione diastereomeric pair 1 (25 mg, 0.04438 mmol) was purified on a preparative LUX-3 Chiral SFC column using a gradient from 50-80% methanol (NH₃) to give: Diastereomer 1a (earlier eluting SFC peak on LUX-3 chiral column), 18-(3,3-dimethylbutyl)-12-(2,6-dimethylphenyl)-8-imino-15-oxa-8λ⁶-thia-1,9,11,18,22-pentaazatetracyclo[14.4.1.13,7.110,14]tricosa-3,5,7(23),10,12,14(22)-hexaene-2,8-dione (5.6 mg, 45%), ¹H NMR (400 MHz, DMSO-d₆) δ 8.45 (s, 1H), 7.94 (s, 1H), 7.64 (s, 2H), 7.25-7.13 (m, 1H), 7.05 (d, J=7.6 Hz, 2H), 6.05 (s, 1H), 5.56 (s, 1H), 4.16 (s, 1H), 3.21 (s, 2H), 3.09 (s, 2H), 2.82 (s, 3H), 2.67 (s, 3H), 1.96 (s, 6H), 1.43 (s, 2H), 1.24 (s, 2H), 0.91 (s, 9H). ESI-MS m/z calc. 562.2726, found 563.0 (M+1)⁺; Retention time: 1.29 minutes (LC method I); and diastereomer 1b (later eluting SFC peak on LUX-3 chiral column), 18-(3,3-dimethylbutyl)-12-(2,6-dimethylphenyl)-8-imino-15-oxa-8λ⁶-thia-1,9,11,18,22-pentaazatetracyclo[14.4.1.13,7.110,14]tricosa-3,5,7(23),10,12,14(22)-hexaene-2,8-dione (5.7 mg, 460%) H NMR (400 MHz, DMSO-d₆) δ 8.45 (s, 1H), 7.94 (s, 1H), 7.65 (s, 2H), 7.13 (s, 1H), 7.05 (d, J=7.6 Hz, 2H), 6.05 (s, 1H), 3.46 (d, J=13.3 Hz, 1H), 3.19 (s, 2H), 3.09 (s, 2H), 2.85 (s, 2H), 2.66 (s, 2H), 1.96 (s, 6H), 1.47-1.39 (m, 2H), 1.23 (s, 2H), 0.91 (s, 9H). ESI-MS m/z calc. 562.2726, found 563.0 (M+1)⁺; Retention time: 1.29 minutes (LC method I).

### Example 140: Characterization of Compounds 246-248

The compounds in the following tables were prepared in a manner analogous to that described above using commercially available reagents and intermediates described herein.

TABLE 9

| Cmpd | | LC Rt | Calc. | | LCMS | |
|---|---|---|---|---|---|---|
| number | Structure | (min) | Mass | M + 1 | Met. | NMR |
| 246, diast. 2 | 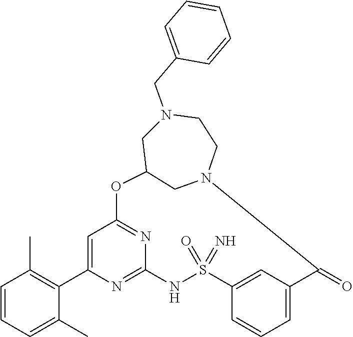 [SMILES: Cc1cccc(C)c1-c1cc2nc(n1)NS(=N)(=O)c1cccc(c1)C(=O)N1CCN(Cc3ccccc3)CC(C1)O2] | 1 | 568.226 | 569 | A | ¹H NMR (400 MHz, DMSO-d₆) δ 11.10 (s, 1H), 8.86 (s, 1H), 8.64 (s, 2H), 8.16 (s, 1H), 7.76 (s, 4H), 7.53 (s, 3H), 7.29 (s, 1H), 7.14 (s, 2H), 6.57 (s, 1H), 5.87 (s, 1H), 4.62 (s, 2H), 4.45 (s, 3H), 2.05 (s, 7H). |
| 247, diast. 2 | 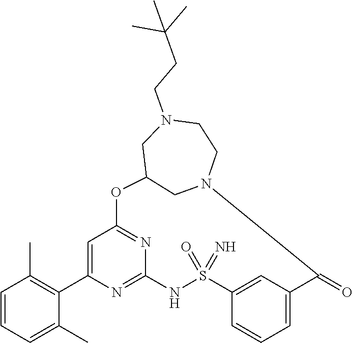 [SMILES: Cc1cccc(C)c1-c1cc2nc(n1)NS(=N)(=O)c1cccc(c1)C(=O)N1CCN(CCC(C)(C)C)CC(C1)O2] | 1.96 | 562.273 | 563 | A | ¹H NMR (400 MHz, DMSO-d₆) δ 10.88 (s, 1H), 8.91 (s, 1H), 8.60 (s, 2H), 8.18 (d, J = 7.8 Hz, 1H), 7.86-7.68 (m, 2H), 7.29 (s, 1H), 7.15 (d, J = 7.5 Hz, 2H), 6.54 (s, 1H), 5.85 (s, 1H), 4.47 (s, 1H), 3.82 (s, 2H), 3.69 (s, 1H), 3.55 (s, 4H), 2.05 (s, 7H), 1.73 (d, J = 7.5 Hz, 2H), 0.95 (s, 10H). |
| 248, diast. 1 | 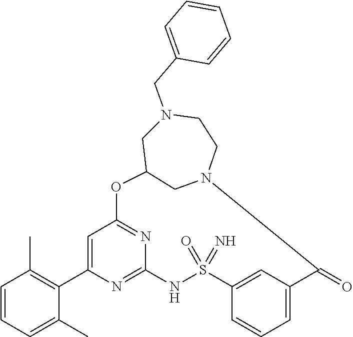 [SMILES: Cc1cccc(C)c1-c1cc2nc(n1)NS(=N)(=O)c1cccc(c1)C(=O)N1CCN(Cc3ccccc3)CC(C1)O2] | 0.98 | 568.226 | 569 | A | ¹H NMR (400 MHz, DMSO-d₆) δ 12.43 (s, 1H), 9.12 (s, 1H), 8.93 (s, 2H), 8.08 (d, J = 8.7 Hz, 1H), 7.78 (d, J = 4.6 Hz, 4H), 7.51 (s, 3H), 7.33 (t, J = 7.6 Hz, 1H), 7.18 (s, 2H), 6.69 (s, 1H), 6.43 (s, 1H), 4.57 (s, 1H), 4.51 (s, 1H), 3.88 (s, 2H), 3.53 (s, 2H), 3.37 (s, 1H), 3.17 (s, 2H), 2.09 (d, J = 27.5 Hz, 6H). |

### Example 141: Characterization of Compounds 249-371

TABLE 10

| | | LCMS | | | |
|---|---|---|---|---|---|
| Compound | | Rt | Calc. | | LCMS |
| number | Structure | (min) | Mass | M + 1 | Method |
| 249 | 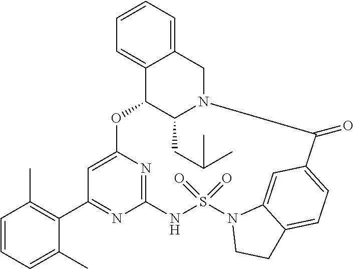 [SMILES: Cc1cccc(C)c1-c1cc2nc(n1)NS(=O)(=O)N1CCc3ccc(cc31)C(=O)N1Cc3ccccc3[C@@H](O2)[C@H]1CC(C)C] | 1.95 | 609.241 | 610.39 | A |
| 250 | 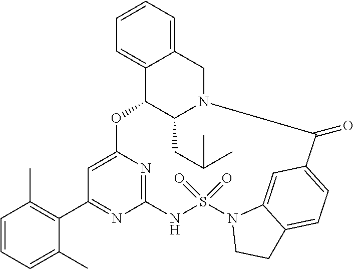 [SMILES: Cc1cccc(C)c1-c1cc2nc(n1)NS(=O)(=O)N1CCc3ccc(cc31)C(=O)N1Cc3ccccc3[C@@H](O2)[C@H]1CC(C)C] | 2.12 | 609.241 | 610.32 | A |
| 251 | 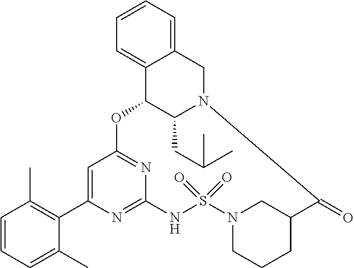 [SMILES: Cc1cccc(C)c1-c1cc2nc(n1)NS(=O)(=O)N1CCCC(C1)C(=O)N1Cc3ccccc3[C@@H](O2)[C@H]1CC(C)C] | 1.44 | 575.257 | 576.1 | A |
| 252 | 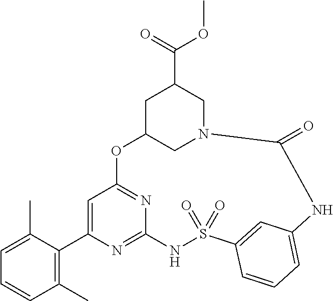 [SMILES: COC(=O)C1CC2CN(C1)C(=O)Nc1cccc(c1)S(=O)(=O)Nc1nc(cc(-c3c(C)cccc3C)n1)O2] | 1.4 | 537.168 | 538.3 | A |
| 253 | 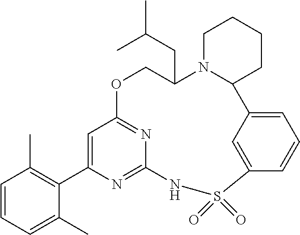 [SMILES: Cc1cccc(C)c1-c1cc2nc(n1)NS(=O)(=O)c1cccc(c1)C1CCCCN1C(CC(C)C)CO2] | 1.14 | 520.251 | 521.5 | A |
| 254 | 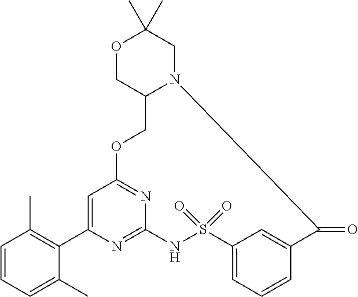 [SMILES: Cc1cccc(C)c1-c1cc2nc(n1)NS(=O)(=O)c1cccc(c1)C(=O)N1CC(C)(C)OCC1CO2] | 1.46 | 508.178 | 509.4 | A |
| 255 | 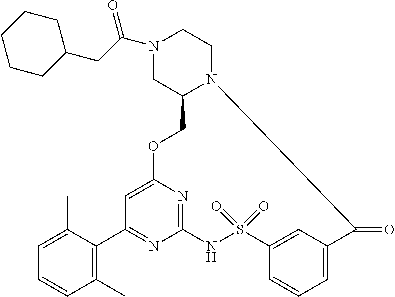 [SMILES: Cc1cccc(C)c1-c1cc2nc(n1)NS(=O)(=O)c1cccc(c1)C(=O)N1CCN(C(=O)CC3CCCCC3)C[C@@H]1CO2] | 1.7 | 603.252 | 604.4 | A |
| 256 | 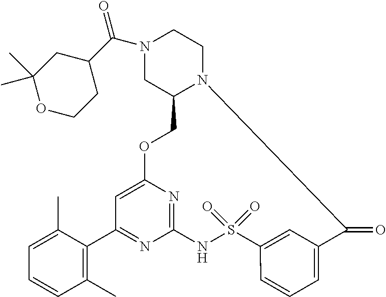 [SMILES: Cc1cccc(C)c1-c1cc2nc(n1)NS(=O)(=O)c1cccc(c1)C(=O)N1CCN(C(=O)C3CCOC(C)(C)C3)C[C@@H]1CO2] | 1.1 | 605.267 | 606.4 | A |
| 257 | 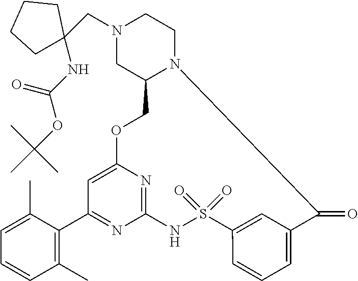 [SMILES: Cc1cccc(C)c1-c1cc2nc(n1)NS(=O)(=O)c1cccc(c1)C(=O)N1CCN(CC3(NC(=O)OC(C)(C)C)CCCC3)C[C@@H]1CO2] | 1.48 | 676.304 | 677.5 | A |
| 258 | 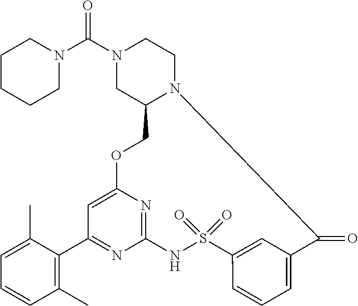 [SMILES: Cc1cccc(C)c1-c1cc2nc(n1)NS(=O)(=O)c1cccc(c1)C(=O)N1CCN(C(=O)N3CCCCC3)C[C@@H]1CO2] | 1.47 | 590.231 | 591.3 | A |
| 259 | 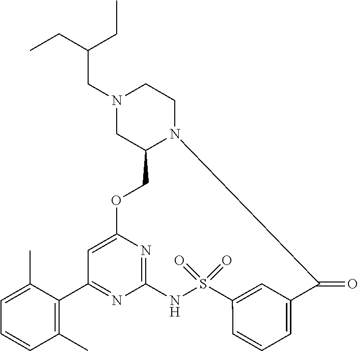 [SMILES: CCC(CC)CN1CCN2C(=O)c3cccc(c3)S(=O)(=O)Nc3nc(cc(-c4c(C)cccc4C)n3)OC[C@H]2C1] | 1.25 | 563.257 | 564.32 | A |
| 260 | 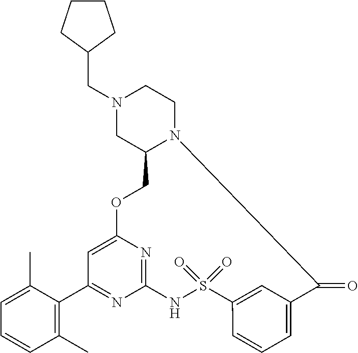 [SMILES: Cc1cccc(C)c1-c1cc2nc(n1)NS(=O)(=O)c1cccc(c1)C(=O)N1CCN(CC3CCCC3)C[C@@H]1CO2] | 1.17 | 561.241 | 562.24 | A |
| 261 | 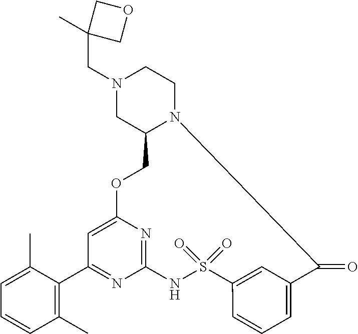 [SMILES: Cc1cccc(C)c1-c1cc2nc(n1)NS(=O)(=O)c1cccc(c1)C(=O)N1CCN(CC3(C)COC3)C[C@@H]1CO2] | 1 | 563.22 | 564.26 | A |
| 262 | 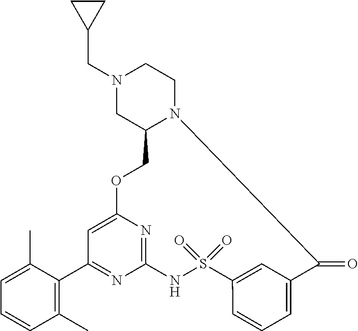 [SMILES: Cc1cccc(C)c1-c1cc2nc(n1)NS(=O)(=O)c1cccc(c1)C(=O)N1CCN(CC3CC3)C[C@@H]1CO2] | 1 | 533.21 | 534.28 | A |
| 263 | 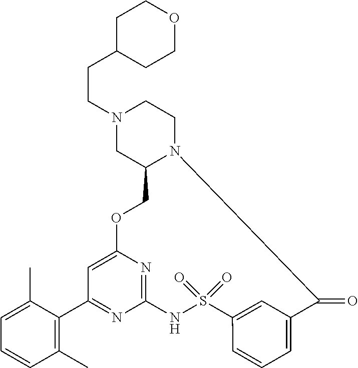 [SMILES: Cc1cccc(C)c1-c1cc2nc(n1)NS(=O)(=O)c1cccc(c1)C(=O)N1CCN(CCC3CCOCC3)C[C@@H]1CO2] | 1 | 591.252 | 592.28 | A |
| 264 | 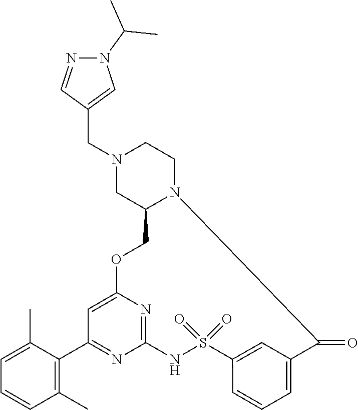 [SMILES: Cc1cccc(C)c1-c1cc2nc(n1)NS(=O)(=O)c1cccc(c1)C(=O)N1CCN(Cc3cnn(C(C)C)c3)C[C@@H]1CO2] | 1.03 | 601.247 | 602.3 | A |
| 265 | 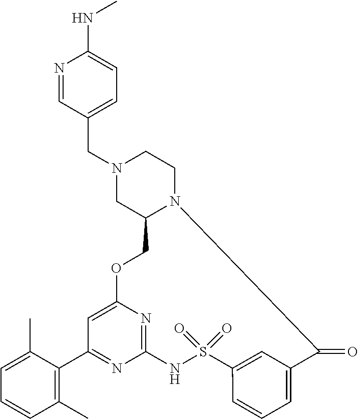 [SMILES: CNc1ccc(CN2CCN3C(=O)c4cccc(c4)S(=O)(=O)Nc4nc(cc(-c5c(C)cccc5C)n4)OC[C@H]3C2)cn1] | 0.89 | 599.231 | 600.28 | A |
| 266 | 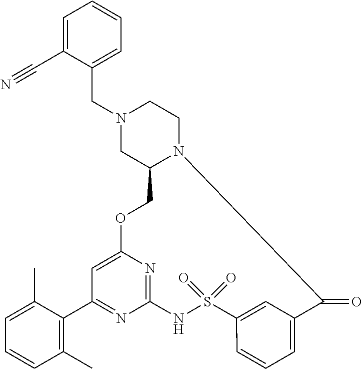 [SMILES: Cc1cccc(C)c1-c1cc2nc(n1)NS(=O)(=O)c1cccc(c1)C(=O)N1CCN(Cc3ccccc3C#N)C[C@@H]1CO2] | 1.465 | 594.205 | 595.285 | A |
| 267 | 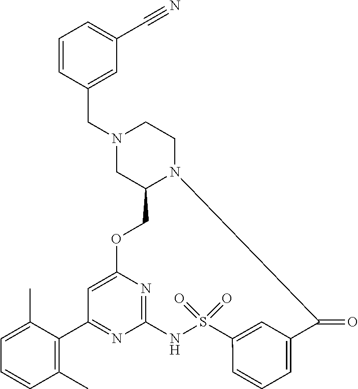 [SMILES: Cc1cccc(C)c1-c1cc2nc(n1)NS(=O)(=O)c1cccc(c1)C(=O)N1CCN(Cc3cccc(C#N)c3)C[C@@H]1CO2] | 1.28 | 594.205 | 595.275 | A |
| 268 | 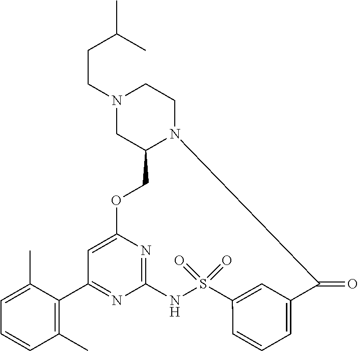 [SMILES: Cc1cccc(C)c1-c1cc2nc(n1)NS(=O)(=O)c1cccc(c1)C(=O)N1CCN(CCC(C)C)C[C@@H]1CO2] | 1.16 | 549.241 | 550.27 | A |
| 269 | 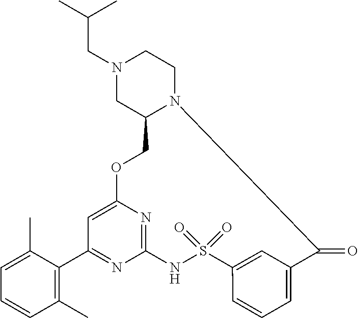 [SMILES: Cc1cccc(C)c1-c1cc2nc(n1)NS(=O)(=O)c1cccc(c1)C(=O)N1CCN(CC(C)C)C[C@@H]1CO2] | 1.06 | 535.225 | 536.29 | A |
| 270 | 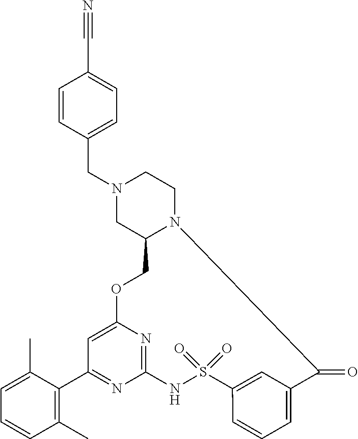 [SMILES: Cc1cccc(C)c1-c1cc2nc(n1)NS(=O)(=O)c1cccc(c1)C(=O)N1CCN(Cc3ccc(C#N)cc3)C[C@@H]1CO2] | 1.28 | 594.205 | 595.275 | A |
| 271 | 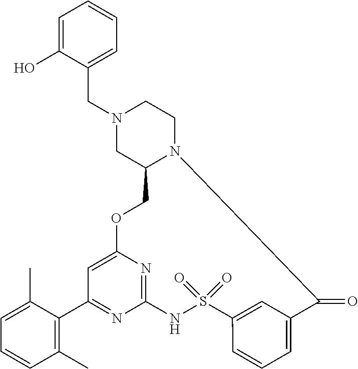 [SMILES: Cc1cccc(C)c1-c1cc2nc(n1)NS(=O)(=O)c1cccc(c1)C(=O)N1CCN(Cc3ccccc3O)C[C@@H]1CO2] | 1.1 | 585.205 | 586.23 | A |
| 272 | 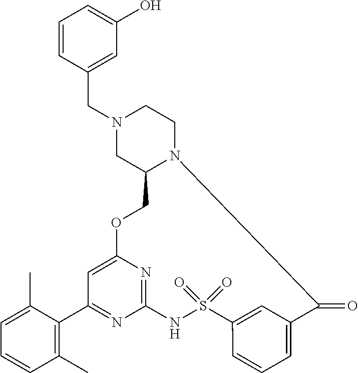 [SMILES: Cc1cccc(C)c1-c1cc2nc(n1)NS(=O)(=O)c1cccc(c1)C(=O)N1CCN(Cc3cccc(O)c3)C[C@@H]1CO2] | 1.04 | 585.205 | 586.23 | A |
| 273 | 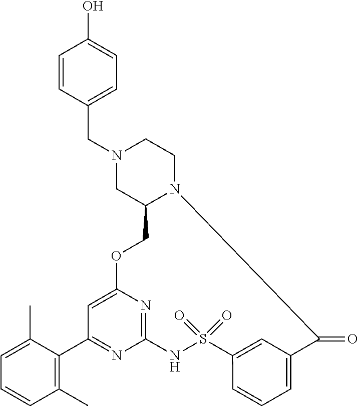 [SMILES: Cc1cccc(C)c1-c1cc2nc(n1)NS(=O)(=O)c1cccc(c1)C(=O)N1CCN(Cc3ccc(O)cc3)C[C@@H]1CO2] | 1 | 585.205 | 586.23 | A |
| 274 | 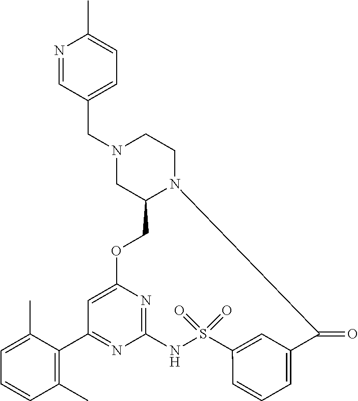 [SMILES: Cc1ccc(CN2CCN3C(=O)c4cccc(c4)S(=O)(=O)Nc4nc(cc(-c5c(C)cccc5C)n4)OC[C@H]3C2)cn1] | 0.92 | 584.221 | 585.5 | A |
| 275 | 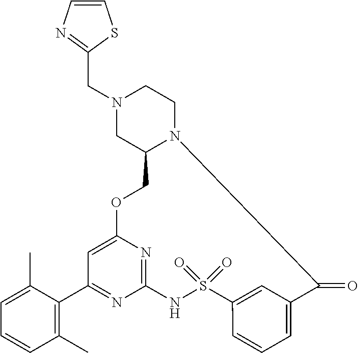 [SMILES: Cc1cccc(C)c1-c1cc2nc(n1)NS(=O)(=O)c1cccc(c1)C(=O)N1CCN(Cc3nccs3)C[C@@H]1CO2] | 1.22 | 576.161 | 577.16 | A |
| 276 | 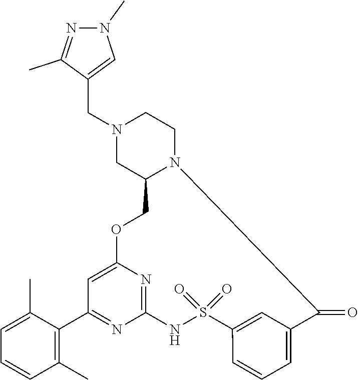 [SMILES: Cc1cccc(C)c1-c1cc2nc(n1)NS(=O)(=O)c1cccc(c1)C(=O)N1CCN(Cc3cn(C)nc3C)C[C@@H]1CO2] | 0.97 | 587.231 | 588.25 | A |
| 277 | 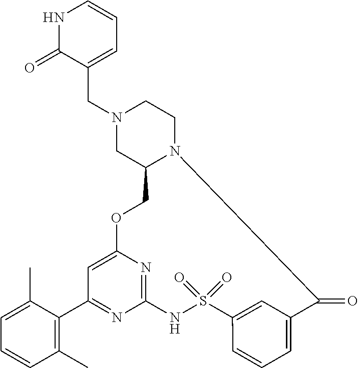 [SMILES: Cc1cccc(C)c1-c1cc2nc(n1)NS(=O)(=O)c1cccc(c1)C(=O)N1CCN(Cc3ccc[nH]c3=O)C[C@@H]1CO2] | 0.88 | 586.2 | 587.24 | A |
| 278 | 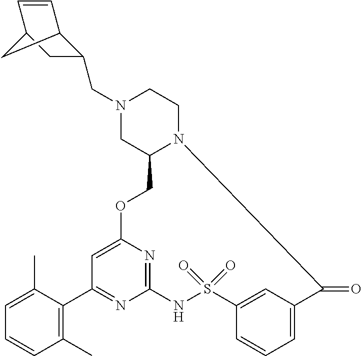 [SMILES: Cc1cccc(C)c1-c1cc2nc(n1)NS(=O)(=O)c1cccc(c1)C(=O)N1CCN(CC3CC4C=CC3C4)C[C@@H]1CO2] | 1.23 | 585.241 | 586.3 | A |
| 279 | 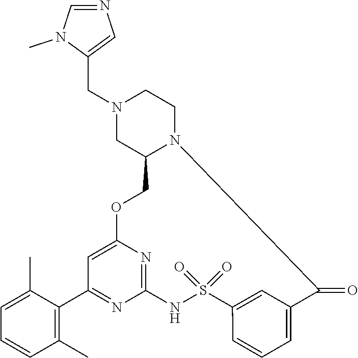 [SMILES: Cc1cccc(C)c1-c1cc2nc(n1)NS(=O)(=O)c1cccc(c1)C(=O)N1CCN(Cc3cncn3C)C[C@@H]1CO2] | 0.96 | 573.216 | 574.27 | A |
| 280 | 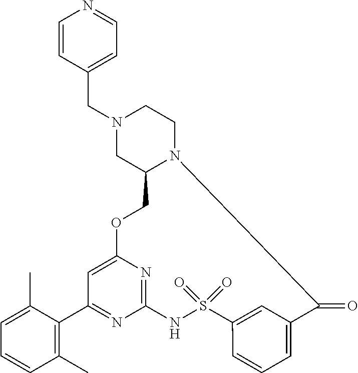 [SMILES: Cc1cccc(C)c1-c1cc2nc(n1)NS(=O)(=O)c1cccc(c1)C(=O)N1CCN(Cc3ccncc3)C[C@@H]1CO2] | 0.995 | 570.205 | 571.09 | A |
| 281 | 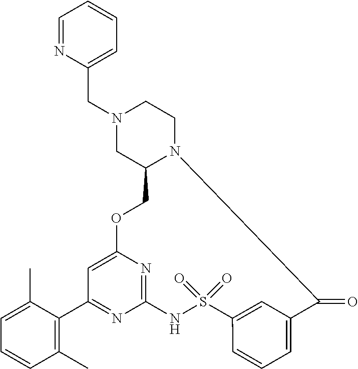 [SMILES: Cc1cccc(C)c1-c1cc2nc(n1)NS(=O)(=O)c1cccc(c1)C(=O)N1CCN(Cc3ccccn3)C[C@@H]1CO2] | 1 | 570.205 | 571.25 | A |
| 282 | 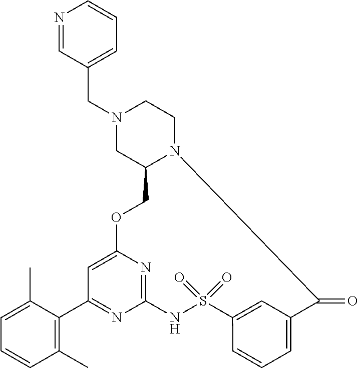 [SMILES: Cc1cccc(C)c1-c1cc2nc(n1)NS(=O)(=O)c1cccc(c1)C(=O)N1CCN(Cc3cccnc3)C[C@@H]1CO2] | 0.995 | 570.205 | 571.125 | A |
| 283 | 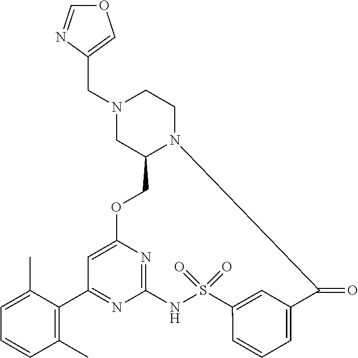 [SMILES: Cc1cccc(C)c1-c1cc2nc(n1)NS(=O)(=O)c1cccc(c1)C(=O)N1CCN(Cc3cocn3)C[C@@H]1CO2] | 0.93 | 560.184 | 561.23 | A |
| 284 | 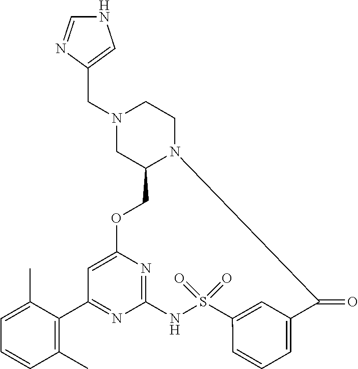 [SMILES: Cc1cccc(C)c1-c1cc2nc(n1)NS(=O)(=O)c1cccc(c1)C(=O)N1CCN(Cc3c[nH]cn3)C[C@@H]1CO2] | 0.89 | 559.2 | 560.22 | A |
| 285 | 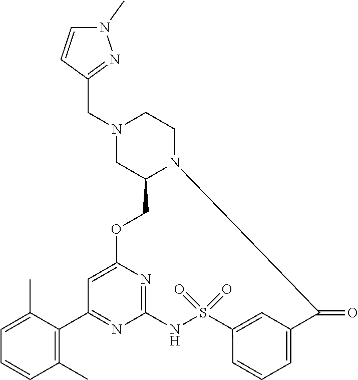 [SMILES: Cc1cccc(C)c1-c1cc2nc(n1)NS(=O)(=O)c1cccc(c1)C(=O)N1CCN(Cc3ccn(C)n3)C[C@@H]1CO2] | 0.96 | 573.216 | 574.27 | A |
| 286 | 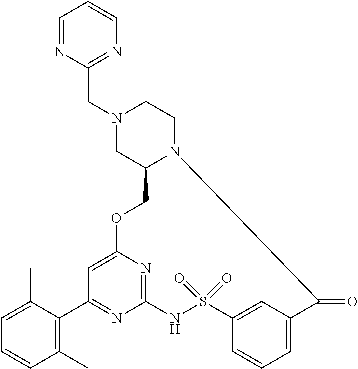 [SMILES: Cc1cccc(C)c1-c1cc2nc(n1)NS(=O)(=O)c1cccc(c1)C(=O)N1CCN(Cc3ncccn3)C[C@@H]1CO2] | 0.95 | 571.2 | 572.25 | A |
| 287 | 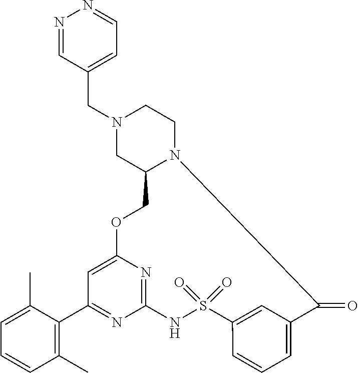 [SMILES: Cc1cccc(C)c1-c1cc2nc(n1)NS(=O)(=O)c1cccc(c1)C(=O)N1CCN(Cc3ccnnc3)C[C@@H]1CO2] | 1.07 | 571.2 | 572.25 | A |
| 288 | 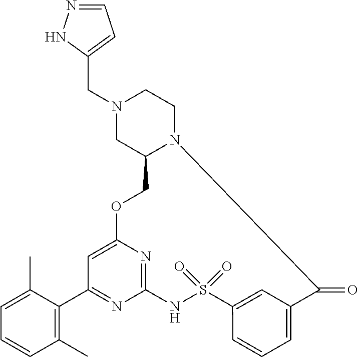 [SMILES: Cc1cccc(C)c1-c1cc2nc(n1)NS(=O)(=O)c1cccc(c1)C(=O)N1CCN(Cc3ccn[nH]3)C[C@@H]1CO2] | 0.9 | 559.2 | 560.22 | A |
| 289 | 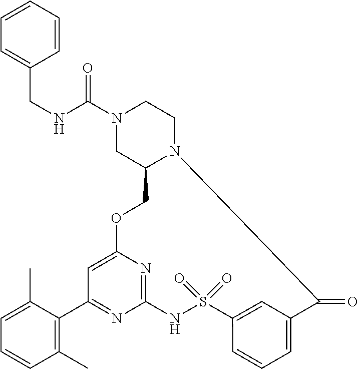 [SMILES: Cc1cccc(C)c1-c1cc2nc(n1)NS(=O)(=O)c1cccc(c1)C(=O)N1CCN(C(=O)NCc3ccccc3)C[C@@H]1CO2] | 1.41 | 612.215 | 613.3 | A |
| 290 | 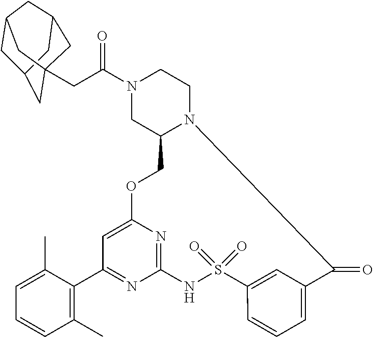 [SMILES: Cc1cccc(C)c1-c1cc2nc(n1)NS(=O)(=O)c1cccc(c1)C(=O)N1CCN(C(=O)CC34CC5CC(CC(C5)C3)C4)C[C@@H]1CO2] | 1.8 | 656.278 | 657.4 | A |
| 291 | 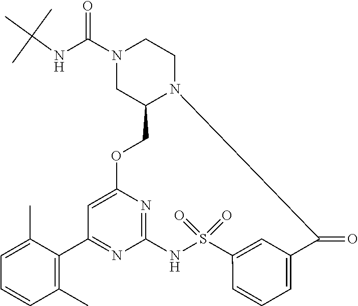 [SMILES: Cc1cccc(C)c1-c1cc2nc(n1)NS(=O)(=O)c1cccc(c1)C(=O)N1CCN(C(=O)NC(C)(C)C)C[C@@H]1CO2] | 1.44 | 578.231 | 579.4 | A |
| 292 | 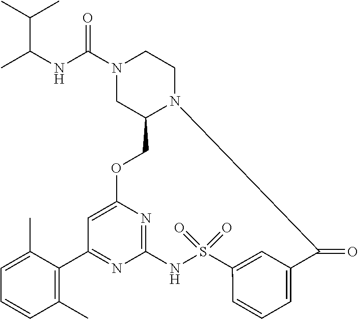 [SMILES: Cc1cccc(C)c1-c1cc2nc(n1)NS(=O)(=O)c1cccc(c1)C(=O)N1CCN(C(=O)NC(C)C(C)C)C[C@@H]1CO2] | 1.46 | 592.247 | 593.4 | A |
| 293 | 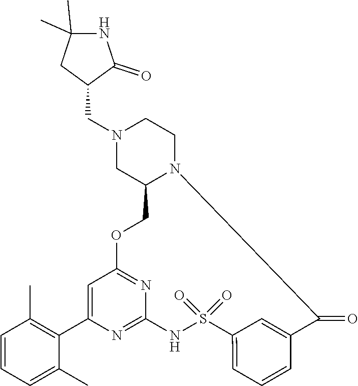 [SMILES: Cc1cccc(C)c1-c1cc2nc(n1)NS(=O)(=O)c1cccc(c1)C(=O)N1CCN(C[C@@H]3CC(C)(C)NC3=O)C[C@@H]1CO2] | 0.94 | 604.247 | 605.4 | A |
| 294 | 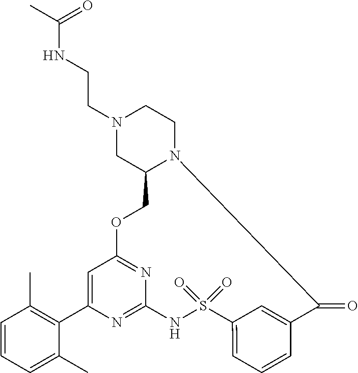 [SMILES: CC(=O)NCCN1CCN2C(=O)c3cccc(c3)S(=O)(=O)Nc3nc(cc(-c4c(C)cccc4C)n3)OC[C@H]2C1] | 0.8 | 564.215 | 565.4 | A |
| 295 | 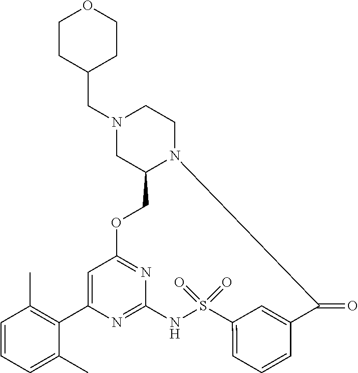 [SMILES: Cc1cccc(C)c1-c1cc2nc(n1)NS(=O)(=O)c1cccc(c1)C(=O)N1CCN(CC3CCOCC3)C[C@@H]1CO2] | 0.92 | 577.236 | 578.4 | A |
| 296 | 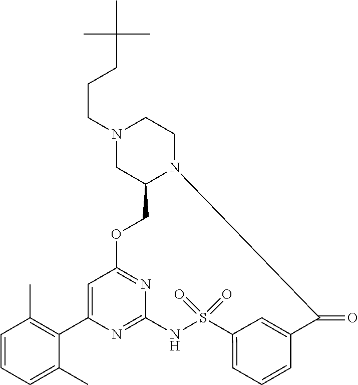 [SMILES: Cc1cccc(C)c1-c1cc2nc(n1)NS(=O)(=O)c1cccc(c1)C(=O)N1CCN(CCCC(C)(C)C)C[C@@H]1CO2] | 1.3 | 577.272 | 578.4 | A |
| 297 | 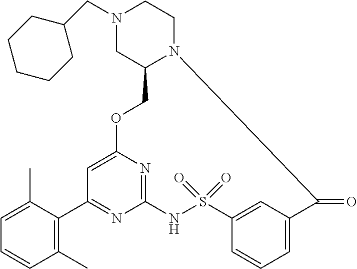 [SMILES: Cc1cccc(C)c1-c1cc2nc(n1)NS(=O)(=O)c1cccc(c1)C(=O)N1CCN(CC3CCCCC3)C[C@@H]1CO2] | 1.25 | 575.257 | 576.4 | A |
| 298 | 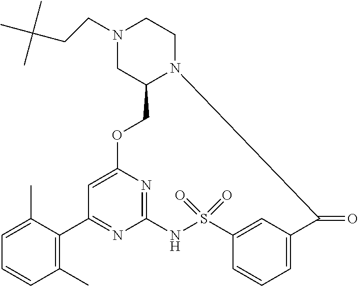 [SMILES: Cc1cccc(C)c1-c1cc2nc(n1)NS(=O)(=O)c1cccc(c1)C(=O)N1CCN(CCC(C)(C)C)C[C@@H]1CO2] | 1.14 | 563.257 | 564.35 | A |
| 299 | 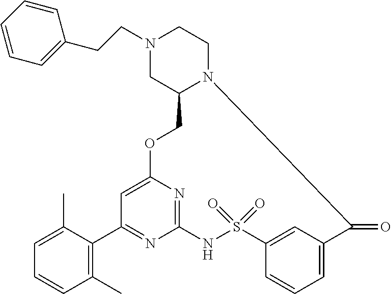 [SMILES: Cc1cccc(C)c1-c1cc2nc(n1)NS(=O)(=O)c1cccc(c1)C(=O)N1CCN(CCc3ccccc3)C[C@@H]1CO2] | 1.17 | 583.225 | 584.4 | A |
| 300 | 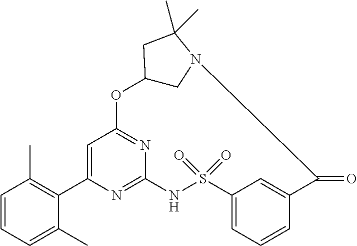 [SMILES: Cc1cccc(C)c1-c1cc2nc(n1)NS(=O)(=O)c1cccc(c1)C(=O)N1CC(CC1(C)C)O2] | 1.51 | 478.167 | 479.3 | A |
| 301 | 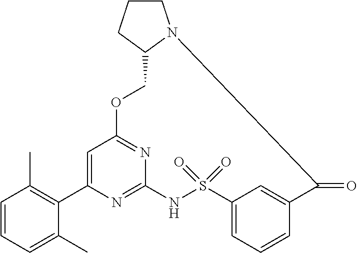 | 1.31 | 464.152 | 465.3 | A |
| 302 | 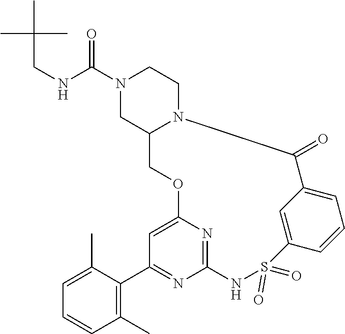 [SMILES: Cc1cccc(C)c1-c1cc2nc(n1)NS(=O)(=O)c1cccc(c1)C(=O)N1CCN(C(=O)NCC(C)(C)C)CC1CO2] | 1.41 | 592.247 | 593.4 | A |
| 303 | 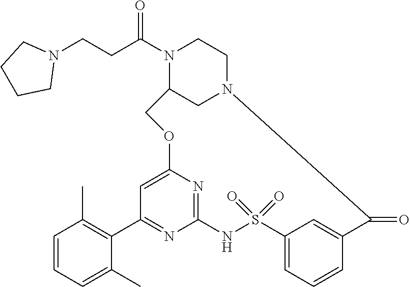 [SMILES: Cc1cccc(C)c1-c1cc2nc(n1)NS(=O)(=O)c1cccc(c1)C(=O)N1CCN(C(=O)CCN3CCCC3)C(CO2)C1] | 0.83 | 604.247 | 605.4 | A |
| 304 | 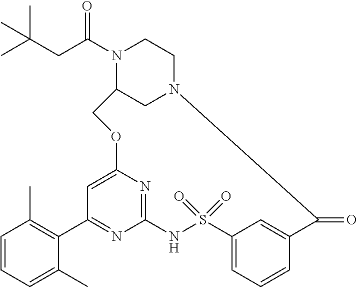 [SMILES: Cc1cccc(C)c1-c1cc2nc(n1)NS(=O)(=O)c1cccc(c1)C(=O)N1CCN(C(=O)CC(C)(C)C)C(CO2)C1] | 1.51 | 577.236 | 578.4 | A |
| 305 | 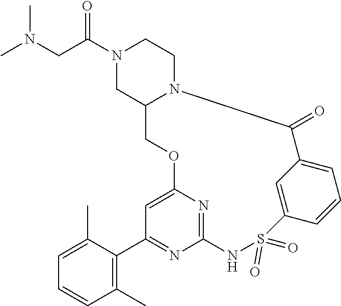 [SMILES: Cc1cccc(C)c1-c1cc2nc(n1)NS(=O)(=O)c1cccc(c1)C(=O)N1CCN(C(=O)CN(C)C)CC1CO2] | 0.72 | 564.215 | 565.3 | A |
| 306 | 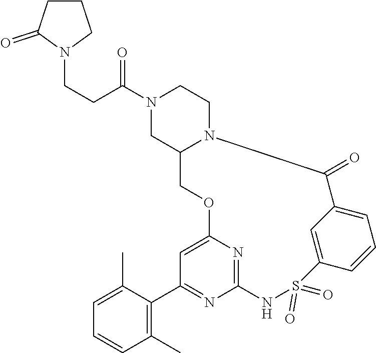 [SMILES: Cc1cccc(C)c1-c1cc2nc(n1)NS(=O)(=O)c1cccc(c1)C(=O)N1CCN(C(=O)CCN3CCCC3=O)CC1CO2] | 1.02 | 618.226 | 619.3 | A |
| 307 | 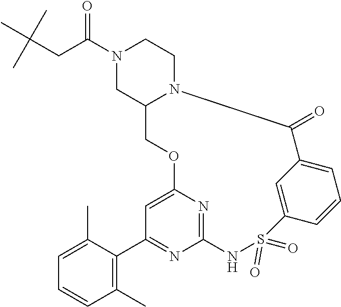 [SMILES: Cc1cccc(C)c1-c1cc2nc(n1)NS(=O)(=O)c1cccc(c1)C(=O)N1CCN(C(=O)CC(C)(C)C)CC1CO2] | 1.48 | 577.236 | 578.4 | A |
| 308 | 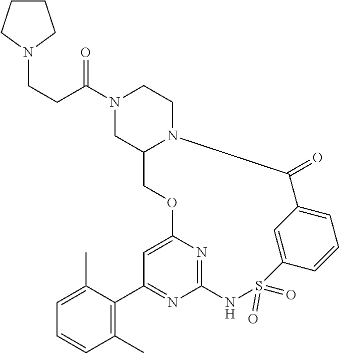 [SMILES: Cc1cccc(C)c1-c1cc2nc(n1)NS(=O)(=O)c1cccc(c1)C(=O)N1CCN(C(=O)CCN3CCCC3)CC1CO2] | 0.8 | 604.247 | 605.4 | A |
| 309 | 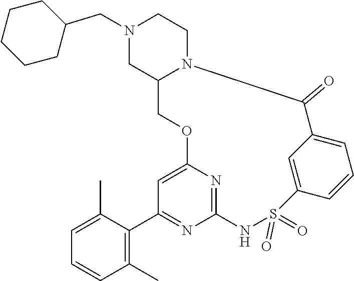 [SMILES: Cc1cccc(C)c1-c1cc2nc(n1)NS(=O)(=O)c1cccc(c1)C(=O)N1CCN(CC3CCCCC3)CC1CO2] | 1.18 | 575.257 | 576.3 | A |
| 310 | 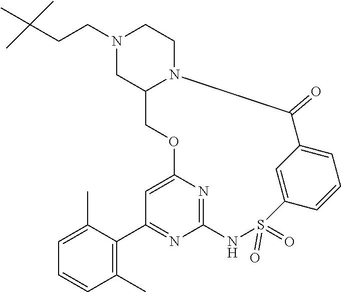 [SMILES: Cc1cccc(C)c1-c1cc2nc(n1)NS(=O)(=O)c1cccc(c1)C(=O)N1CCN(CCC(C)(C)C)CC1CO2] | 1.12 | 563.257 | 564.4 | A |
| 311 | 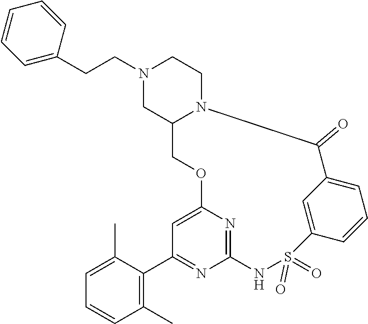 [SMILES: Cc1cccc(C)c1-c1cc2nc(n1)NS(=O)(=O)c1cccc(c1)C(=O)N1CCN(CCc3ccccc3)CC1CO2] | 1.11 | 583.225 | 584.3 | A |
| 312 | 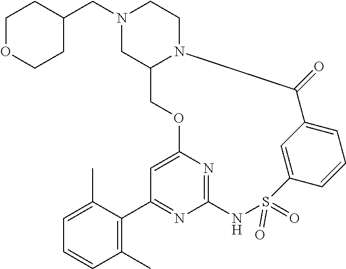 [SMILES: Cc1cccc(C)c1-c1cc2nc(n1)NS(=O)(=O)c1cccc(c1)C(=O)N1CCN(CC3CCOCC3)CC1CO2] | 0.87 | 577.236 | 578.4 | A |
| 313 | 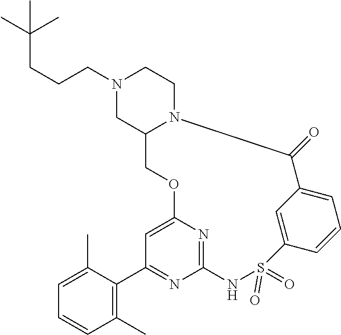 [SMILES: Cc1cccc(C)c1-c1cc2nc(n1)NS(=O)(=O)c1cccc(c1)C(=O)N1CCN(CCCC(C)(C)C)CC1CO2] | 1.23 | 577.272 | 578.4 | A |
| 314 | 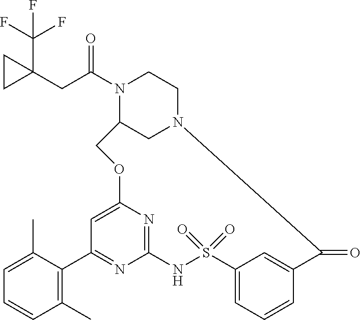 [SMILES: Cc1cccc(C)c1-c1cc2nc(n1)NS(=O)(=O)c1cccc(c1)C(=O)N1CCN(C(=O)CC3(C(F)(F)F)CC3)C(CO2)C1] | 1.24 | 615.213 | 616.4 | A |
| 315 | 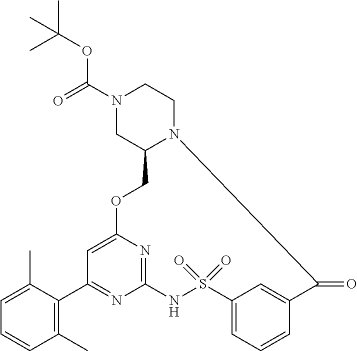 [SMILES: Cc1cccc(C)c1-c1cc2nc(n1)NS(=O)(=O)c1cccc(c1)C(=O)N1CCN(C(=O)OC(C)(C)C)C[C@@H]1CO2] | 1.72 | 579.215 | 580.3 | A |
| 316 | 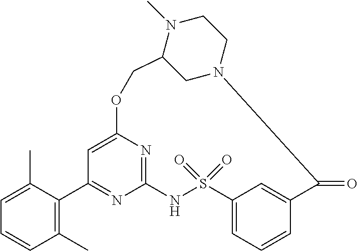 [SMILES: Cc1cccc(C)c1-c1cc2nc(n1)NS(=O)(=O)c1cccc(c1)C(=O)N1CCN(C)C(CO2)C1] | 0.98 | 493.178 | 494.4 | A |
| 317 | 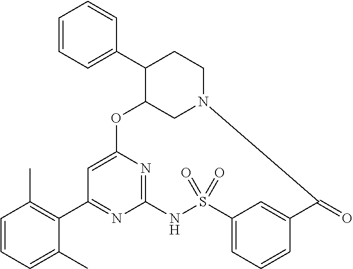 [SMILES: Cc1cccc(C)c1-c1cc2nc(n1)NS(=O)(=O)c1cccc(c1)C(=O)N1CCC(c3ccccc3)C(C1)O2] | 1.69 | 540.183 | 541.3 | A |
| 318 | 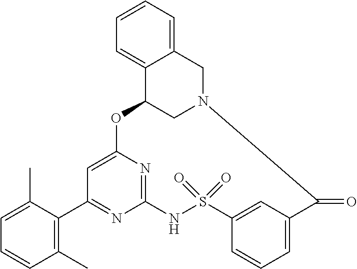 [SMILES: Cc1cccc(C)c1-c1cc2nc(n1)NS(=O)(=O)c1cccc(c1)C(=O)N1Cc3ccccc3[C@@H](C1)O2] | 2.095 | 512.152 | 513.5 | A |
| 319 | 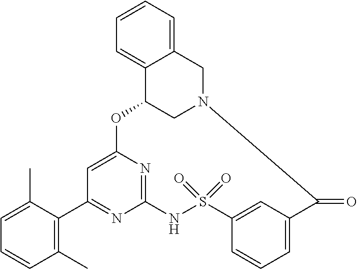 [SMILES: Cc1cccc(C)c1-c1cc2nc(n1)NS(=O)(=O)c1cccc(c1)C(=O)N1Cc3ccccc3[C@H](C1)O2] | 1.71 | 512.152 | 513.3 | A |
| 320 | 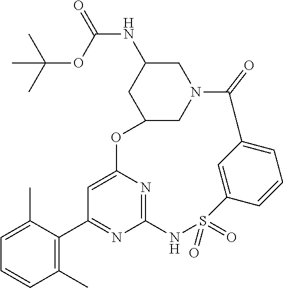 [SMILES: Cc1cccc(C)c1-c1cc2nc(n1)NS(=O)(=O)c1cccc(c1)C(=O)N1CC(NC(=O)OC(C)(C)C)CC(C1)O2] | 1.56 | 579.215 | 580.3 | A |
| 321 | 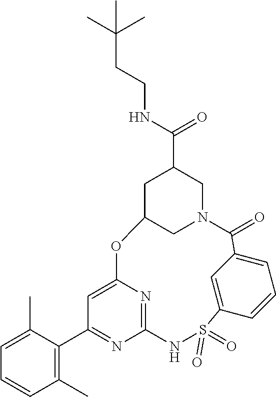 [SMILES: Cc1cccc(C)c1-c1cc2nc(n1)NS(=O)(=O)c1cccc(c1)C(=O)N1CC(CC(C(=O)NCCC(C)(C)C)C1)O2] | 1.66 | 591.252 | 592.3 | A |
| 322 | 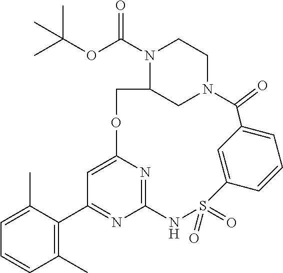 [SMILES: Cc1cccc(C)c1-c1cc2nc(n1)NS(=O)(=O)c1cccc(c1)C(=O)N1CCN(C(=O)OC(C)(C)C)C(CO2)C1] | 1.64 | 579.215 | 580.3 | A |
| 323 | 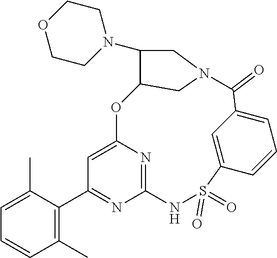 [SMILES: Cc1cccc(C)c1-c1cc2nc(n1)NS(=O)(=O)c1cccc(c1)C(=O)N1CC(O2)C(N2CCOCC2)C1] | 0.95 | 535.189 | 536.2 | A |
| 324 | 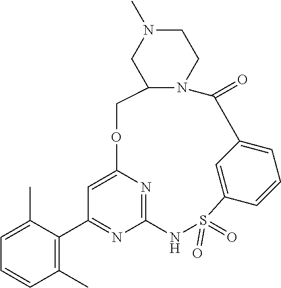 [SMILES: Cc1cccc(C)c1-c1cc2nc(n1)NS(=O)(=O)c1cccc(c1)C(=O)N1CCN(C)CC1CO2] | 0.77 | 493.178 | 494.3 | A |
| 325 | 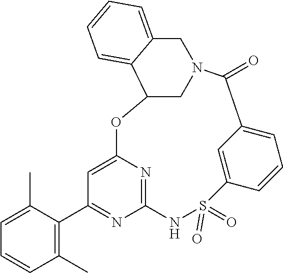 [SMILES: Cc1cccc(C)c1-c1cc2nc(n1)NS(=O)(=O)c1cccc(c1)C(=O)N1Cc3ccccc3C(C1)O2] | 1.62 | 512.152 | 513.2 | A |
| 326 | 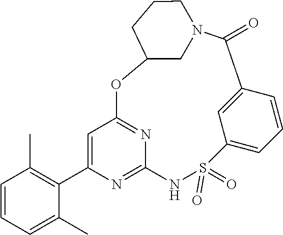 [SMILES: Cc1cccc(C)c1-c1cc2nc(n1)NS(=O)(=O)c1cccc(c1)C(=O)N1CCCC(C1)O2] | 1.41 | 464.152 | 465.2 | A |
| 327 | 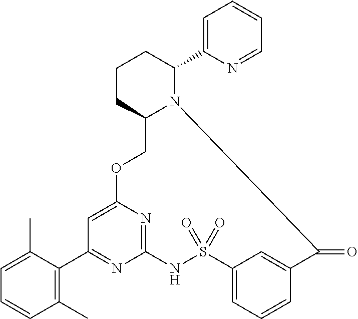 [SMILES: Cc1cccc(C)c1-c1cc2nc(n1)NS(=O)(=O)c1cccc(c1)C(=O)N1[C@H](CCC[C@@H]1c1ccccn1)CO2] | 1.1 | 555.194 | 556.4 | A |
| 328 | 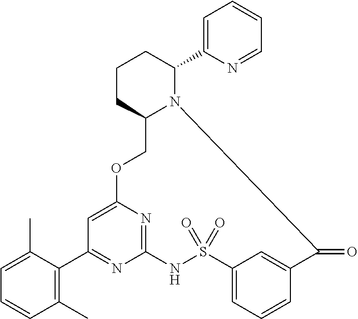 [SMILES: Cc1cccc(C)c1-c1cc2nc(n1)NS(=O)(=O)c1cccc(c1)C(=O)N1[C@H](CCC[C@@H]1c1ccccn1)CO2] | 1.32 | 555.194 | 556.4 | A |
| 329 | 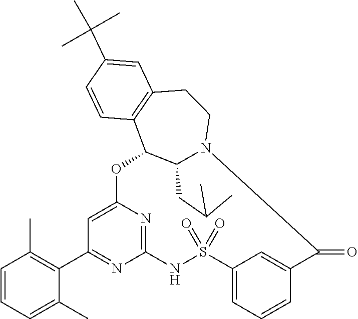 [SMILES: Cc1cccc(C)c1-c1cc2nc(n1)NS(=O)(=O)c1cccc(c1)C(=O)N1CCc3cc(C(C)(C)C)ccc3[C@@H](O2)[C@H]1CC(C)C] | 2.2 | 638.293 | 639.5 | A |
| 330 | 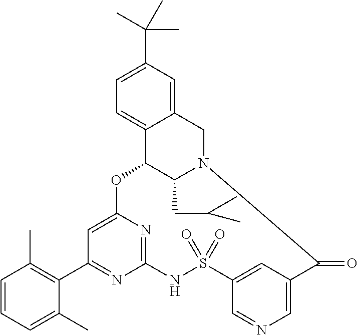 [SMILES: Cc1cccc(C)c1-c1cc2nc(n1)NS(=O)(=O)c1cncc(c1)C(=O)N1Cc3cc(C(C)(C)C)ccc3[C@@H](O2)[C@H]1CC(C)C] | 2.13 | 625.272 | 626.3 | A |
| 331 | 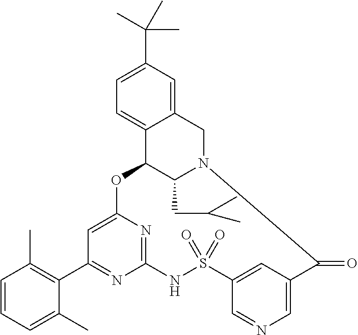 [SMILES: Cc1cccc(C)c1-c1cc2nc(n1)NS(=O)(=O)c1cncc(c1)C(=O)N1Cc3cc(C(C)(C)C)ccc3[C@H](O2)[C@H]1CC(C)C] | 2.065 | 625.272 | 626.35 | A |
| 332 | 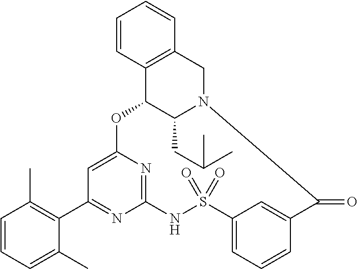 [SMILES: Cc1cccc(C)c1-c1cc2nc(n1)NS(=O)(=O)c1cccc(c1)C(=O)N1Cc3ccccc3[C@@H](O2)[C@H]1CC(C)C] | 1.95 | 568.214 | 569.4 | A |
| 333 | 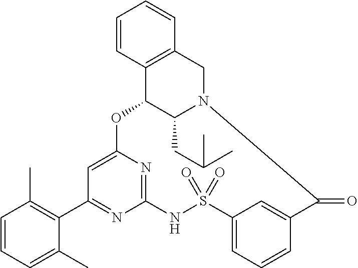 [SMILES: Cc1cccc(C)c1-c1cc2nc(n1)NS(=O)(=O)c1cccc(c1)C(=O)N1Cc3ccccc3[C@@H](O2)[C@H]1CC(C)C] | 1.84 | 568.214 | 569.4 | A |
| 334 | 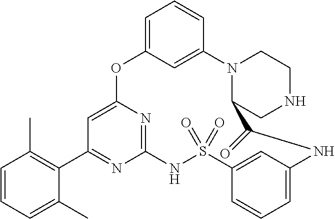 [SMILES: Cc1cccc(C)c1-c1cc2nc(n1)NS(=O)(=O)c1cccc(c1)NC(=O)[C@@H]1CNCCN1c1cccc(c1)O2] | 1.06 | 556.189 | 557.3 | A |
| 335 | 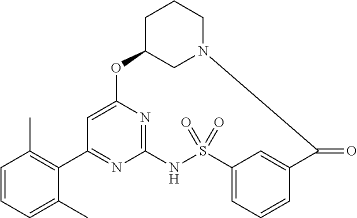 [SMILES: Cc1cccc(C)c1-c1cc2nc(n1)NS(=O)(=O)c1cccc(c1)C(=O)N1CCC[C@@H](C1)O2] | 1.36 | 464.152 | 465.1 | A |
| 336 | 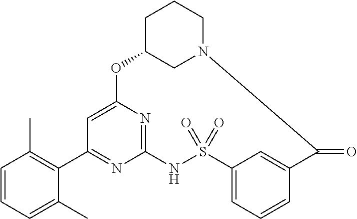 [SMILES: Cc1cccc(C)c1-c1cc2nc(n1)NS(=O)(=O)c1cccc(c1)C(=O)N1CCC[C@H](C1)O2] | 1.36 | 464.152 | 465.1 | A |
| 337 | 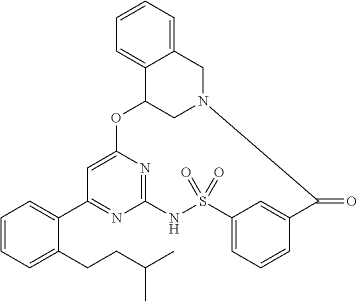 [SMILES: CC(C)CCc1ccccc1-c1cc2nc(n1)NS(=O)(=O)c1cccc(c1)C(=O)N1Cc3ccccc3C(C1)O2] | 2.04 | 554.199 | 555.3 | A |
| 338 | 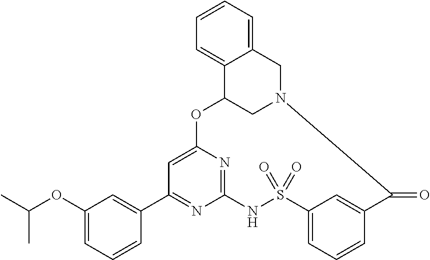 [SMILES: CC(C)Oc1cccc(-c2cc3nc(n2)NS(=O)(=O)c2cccc(c2)C(=O)N2Cc4ccccc4C(C2)O3)c1] | 1.94 | 542.162 | 543.3 | A |
| 339 | 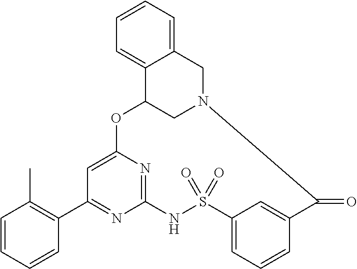 [SMILES: Cc1ccccc1-c1cc2nc(n1)NS(=O)(=O)c1cccc(c1)C(=O)N1Cc3ccccc3C(C1)O2] | 1.64 | 498.136 | 499.2 | A |
| 340 | 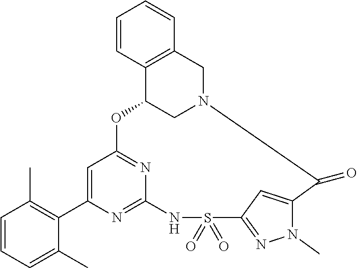 [SMILES: Cc1cccc(C)c1-c1cc2nc(n1)NS(=O)(=O)c1cc(n(C)n1)C(=O)N1Cc3ccccc3[C@H](C1)O2] | 1.47 | 516.158 | 517.3 | A |
| 341 | 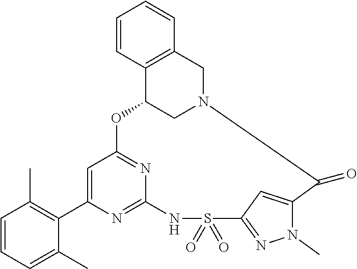 [SMILES: Cc1cccc(C)c1-c1cc2nc(n1)NS(=O)(=O)c1cc(n(C)n1)C(=O)N1Cc3ccccc3[C@H](C1)O2] | 1.47 | 516.158 | 517.3 | A |
| 342 | 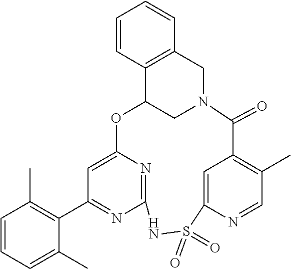 [SMILES: Cc1cnc2cc1C(=O)N1Cc3ccccc3C(C1)Oc1cc(-c3c(C)cccc3C)nc(n1)NS2(=O)=O] | 1.52 | 527.163 | 528.3 | A |
| 343 | 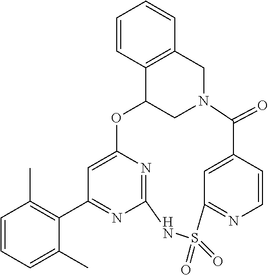 [SMILES: Cc1cccc(C)c1-c1cc2nc(n1)NS(=O)(=O)c1cc(ccn1)C(=O)N1Cc3ccccc3C(C1)O2] | 1.52 | 513.147 | 514.2 | A |
| 344 | 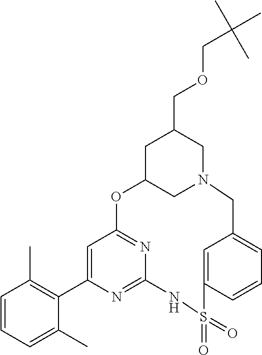 [SMILES: Cc1cccc(C)c1-c1cc2nc(n1)NS(=O)(=O)c1cccc(c1)CN1CC(COCC(C)(C)C)CC(C1)O2] | 1.53 | 550.261 | 551.4 | A |
| 345 | 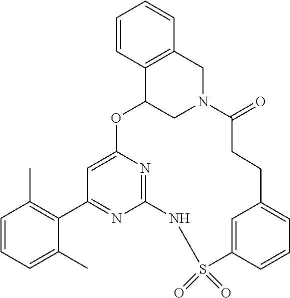 [SMILES: Cc1cccc(C)c1-c1cc2nc(n1)NS(=O)(=O)c1cccc(c1)CCC(=O)N1Cc3ccccc3C(C1)O2] | 1.68 | 540.183 | 541.4 | A |
| 346 | 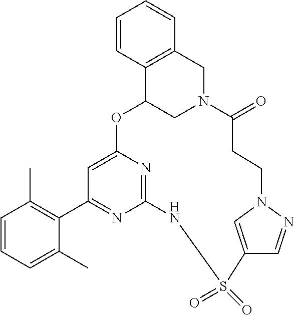 [SMILES: Cc1cccc(C)c1-c1cc2nc(n1)NS(=O)(=O)c1cnn(c1)CCC(=O)N1Cc3ccccc3C(C1)O2] | 1.51 | 530.174 | 531.3 | A |
| 347 | 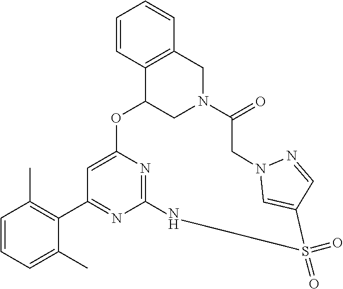 [SMILES: Cc1cccc(C)c1-c1cc2nc(n1)NS(=O)(=O)c1cnn(c1)CC(=O)N1Cc3ccccc3C(C1)O2] | 1.52 | 516.158 | 517.3 | A |
| 348 | 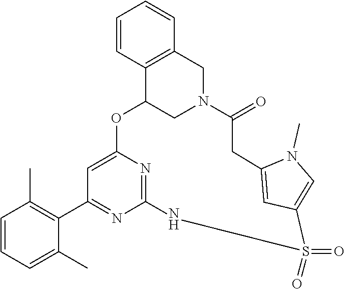 [SMILES: Cc1cccc(C)c1-c1cc2nc(n1)NS(=O)(=O)c1cc(n(C)c1)CC(=O)N1Cc3ccccc3C(C1)O2] | 1.69 | 529.178 | 530.3 | A |
| 349 | 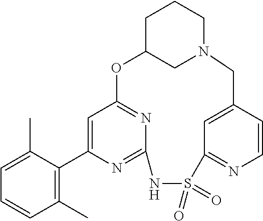 [SMILES: Cc1cccc(C)c1-c1cc2nc(n1)NS(=O)(=O)c1cc(ccn1)CN1CCCC(C1)O2] | 0.8 | 450.173 | 451.3 | A |
| 350 | 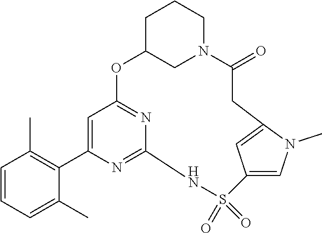 [SMILES: Cc1cccc(C)c1-c1cc2nc(n1)NS(=O)(=O)c1cc(n(C)c1)CC(=O)N1CCCC(C1)O2] | 1.26 | 481.178 | 482.3 | A |
| 351 | 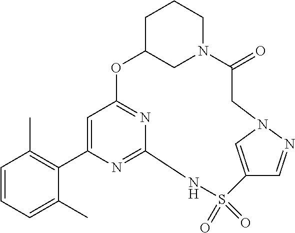 [SMILES: Cc1cccc(C)c1-c1cc2nc(n1)NS(=O)(=O)c1cnn(c1)CC(=O)N1CCCC(C1)O2] | 1.09 | 482.174 | 483.3 | A |
| 352 | 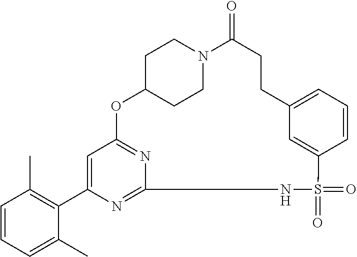 [SMILES: Cc1cccc(C)c1-c1cc2nc(n1)NS(=O)(=O)c1cccc(c1)CCC(=O)N1CCC(CC1)O2] | 1.25 | 492.183 | 493.3 | A |
| 353 | 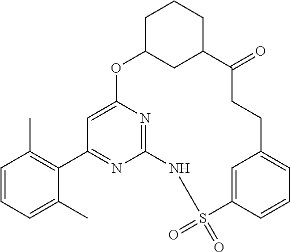 [SMILES: Cc1cccc(C)c1-c1cc2nc(n1)NS(=O)(=O)c1cccc(c1)CCC(=O)C1CCCC(C1)O2] | 1.32 | 492.183 | 493.3 | A |
| 354 | 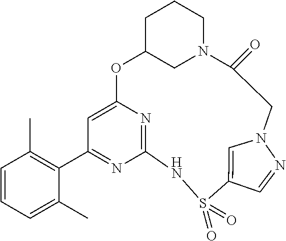 [SMILES: Cc1cccc(C)c1-c1cc2nc(n1)NS(=O)(=O)c1cnn(c1)CC(=O)N1CCCC(C1)O2] | 1.13 | 468.158 | 469.3 | Q |
| 355 | 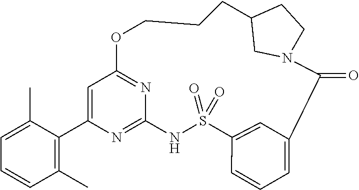 [SMILES: Cc1cccc(C)c1-c1cc2nc(n1)NS(=O)(=O)c1cccc(c1)C(=O)N1CCC(CCCO2)C1] | 1.42 | 492.183 | 493.3 | A |
| 356 | 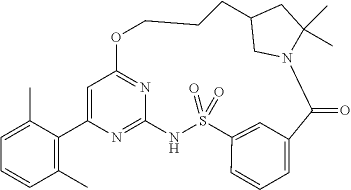 [SMILES: Cc1cccc(C)c1-c1cc2nc(n1)NS(=O)(=O)c1cccc(c1)C(=O)N1CC(CCCO2)CC1(C)C] | 1.69 | 520.214 | 521.2 | A |
| 357 | 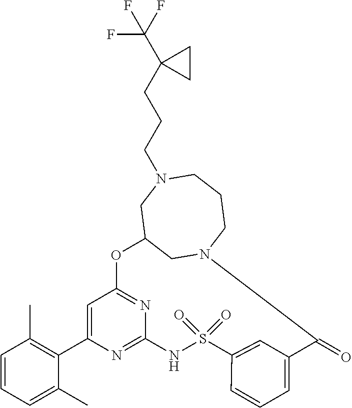 [SMILES: Cc1cccc(C)c1-c1cc2nc(n1)NS(=O)(=O)c1cccc(c1)C(=O)N1CCCN(CCCC3(C(F)(F)F)CC3)CC(C1)O2] | 1.16 | 643.244 | 644 | A |
| 358 | 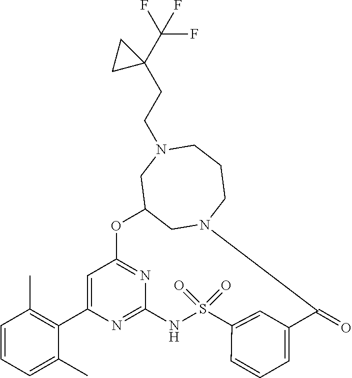 [SMILES: Cc1cccc(C)c1-c1cc2nc(n1)NS(=O)(=O)c1cccc(c1)C(=O)N1CCCN(CCC3(C(F)(F)F)CC3)CC(C1)O2] | 1.15 | 629.228 | 630 | A |
| 359 | 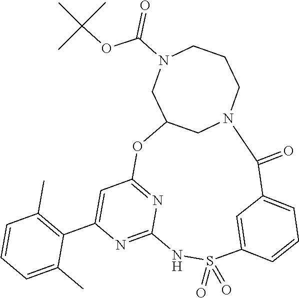 [SMILES: Cc1cccc(C)c1-c1cc2nc(n1)NS(=O)(=O)c1cccc(c1)C(=O)N1CCCN(C(=O)OC(C)(C)C)CC(C1)O2] | 1.52 | 593.231 | 594 | A |
| 360 | 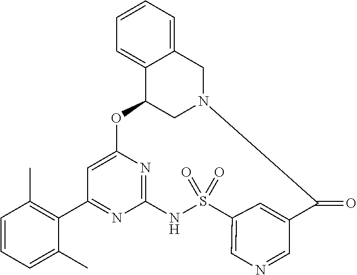 [SMILES: Cc1cccc(C)c1-c1cc2nc(n1)NS(=O)(=O)c1cncc(c1)C(=O)N1Cc3ccccc3[C@@H](C1)O2] | 2.07 | 513.147 | 514 | I |
| 361 | 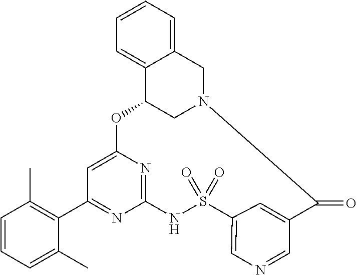 [SMILES: Cc1cccc(C)c1-c1cc2nc(n1)NS(=O)(=O)c1cncc(c1)C(=O)N1Cc3ccccc3[C@H](C1)O2] | 2.07 | 513.147 | 514 | I |
| 362 | 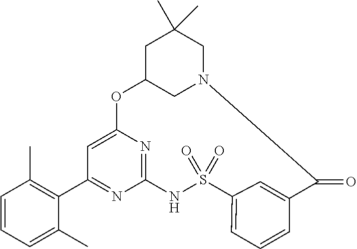 [SMILES: Cc1cccc(C)c1-c1cc2nc(n1)NS(=O)(=O)c1cccc(c1)C(=O)N1CC(CC(C)(C)C1)O2] | 1.52 | 492.183 | 493.4 | A |
| 363 | 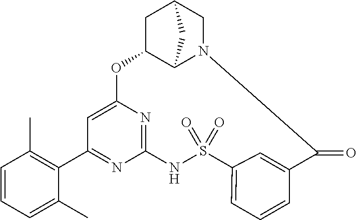 [SMILES: Cc1cccc(C)c1-c1cc2nc(n1)NS(=O)(=O)c1cccc(c1)C(=O)N1C[C@H]3C[C@@H]1[C@@H](C3)O2] | 1.41 | 476.152 | 477.3 | A |
| 364 | 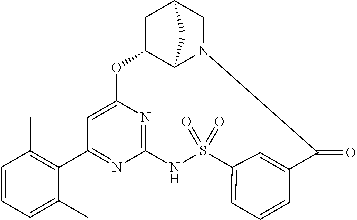 [SMILES: Cc1cccc(C)c1-c1cc2nc(n1)NS(=O)(=O)c1cccc(c1)C(=O)N1C[C@H]3C[C@@H]1[C@@H](C3)O2] | 1.41 | 476.152 | 477.3 | A |
| 365 | 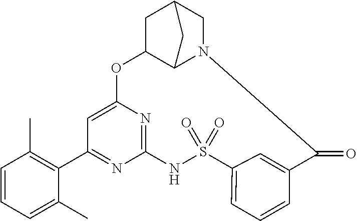 [SMILES: Cc1cccc(C)c1-c1cc2nc(n1)NS(=O)(=O)c1cccc(c1)C(=O)N1CC3CC(O2)C1C3] | 1.39 | 476.152 | 477.3 | A |
| 366 | 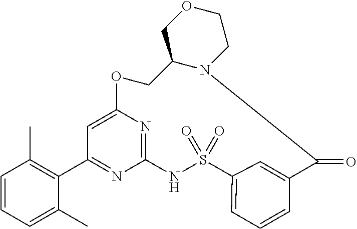 [SMILES: Cc1cccc(C)c1-c1cc2nc(n1)NS(=O)(=O)c1cccc(c1)C(=O)N1CCOC[C@@H]1CO2] | 1.22 | 480.147 | 481.3 | A |
| 367 | 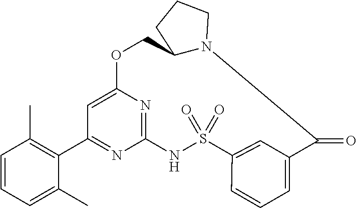 [SMILES: Cc1cccc(C)c1-c1cc2nc(n1)NS(=O)(=O)c1cccc(c1)C(=O)N1CCC[C@@H]1CO2] | 1.31 | 464.152 | 465.3 | A |
| 368 | 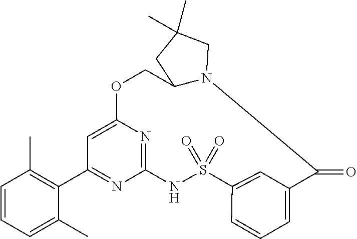 [SMILES: Cc1cccc(C)c1-c1cc2nc(n1)NS(=O)(=O)c1cccc(c1)C(=O)N1CC(C)(C)CC1CO2] | 1.55 | 492.183 | 493.4 | A |
| 369 | 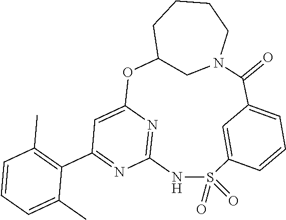 [SMILES: Cc1cccc(C)c1-c1cc2nc(n1)NS(=O)(=O)c1cccc(c1)C(=O)N1CCCCC(C1)O2] | 1.4 | 478.167 | 479.3 | A |
| 370 | 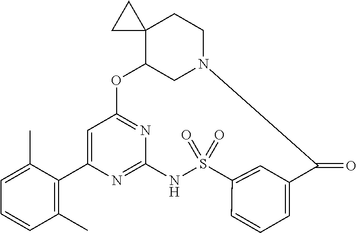 [SMILES: Cc1cccc(C)c1-c1cc2nc(n1)NS(=O)(=O)c1cccc(c1)C(=O)N1CCC3(CC3)C(C1)O2] | 1.61 | 490.167 | 491.3 | A |
| 371 | 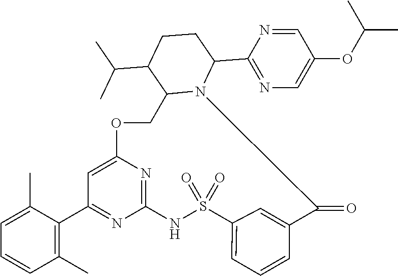 [SMILES: Cc1cccc(C)c1-c1cc2nc(n1)NS(=O)(=O)c1cccc(c1)C(=O)N1C(c3ncc(OC(C)C)cn3)CCC(C(C)C)C1CO2] | 1.97 | 656.278 | 657.5 | A |

TABLE 11

| Com- | |
|---|---|
| pound | |
| number | NMR |
| 253 | ¹H NMR (400 MHZ, DMSO) δ 12.14 (s, 1H), 10.83 (s, 1H), 8.68 (s, |
| | 1H), 8.36 (d, J = 7.6 Hz, 1H), 8.02 (d, J = 7.7 Hz, 1H), 7.80 (t, J = 7.4 |
| | Hz, 1H), 7.22 (t, J = 7.6 Hz, 1H), 7.10 (d, J = 7.6 Hz, 2H), 6.33 (s, 1H), |
| | 5.31 (dd, J = 11.0, 4.4 Hz, 1H), 4.92 (t, J = 10.2 Hz, 1H), 4.33 (t, J = |
| | 11.4 Hz, 1H), 3.62 (d, J = 11.9 Hz, 1H), 3.17 (q, J = 11.5 Hz, 1H), 2.78- |
| | 2.65 (m, 1H), 2.28 (d, J = 14.4 Hz, 1H), 2.17-2.07 (m, 1H), 1.92 (s, |
| | 7H), 1.82-1.62 (m, 3H), 1.36 (q, J = 8.4, 7.8 Hz, 1H), 0.57 (d, J = 6.4 |
| | Hz, 3H), 0.21 (d, J = 6.5 Hz, 3H). (Two protons expected in the |
| | aliphatic region likely overlap with DMSO or water) |
| 298 | ¹H NMR (400 MHZ, DMSO) δ 10.91 (s, 1H), 8.53 (s, 1H), 7.96 (d, J = |
| | 7.0 Hz, 1H), 7.71 (d, J = 9.2 Hz, 2H), 7.27 (t, J = 7.6 Hz, 1H), 7.13 (d, |
| | J = 7.6 Hz, 2H), 6.32 (s, 1H), 5.28 (d, J = 8.4 Hz, 1H), 5.17 (s, 1H), |
| | 4.65 (d, J = 14.8 Hz, 1H), 4.14 (s, 1H), 3.60 (d, J = 11.7 Hz, 1H), 3.35 |
| | (d, J = 13.1 Hz, 2H), 3.20-2.91 (m, 4H), 2.17-2.01 (m, 6H), 1.63 (t, |
| | J = 8.7 Hz, 2H), 0.90 (s, 9H). |
| 318 | ¹H NMR (400 MHZ, DMSO-d₆) δ 8.61 (s, 1H), 7.99 (d, J = 7.5 Hz, 1H), |
| | 7.76 (d, J = 18.5 Hz, 2H), 7.63 (d, J = 7.3 Hz, 1H), 7.45-7.32 (m, 3H), |
| | 7.28 (t, J = 7.6 Hz, 1H), 7.15 (d, J = 7.6 Hz, 2H), 6.48 (d, J = 10.1 Hz, |
| | 2H), 5.28 (d, J = 17.4 Hz, 1H), 4.51-4.42 (m, 1H), 4.12 (dd, J = 13.0, |
| | 4.7 Hz, 1H), 3.18 (s, 1H), 3.16 (dd, J = 13.1, 10.7 Hz, 1H), 2.10 (s, 6H). |
| 329 | ¹H NMR (500 MHZ, DMSO-d₆) δ 13.46-11.57 (broad d, 1H), 7.81 (s, |
| | 1H), 7.71 (d, J = 8.2 Hz, 1H), 7.68-7.43 (m, 3H), 7.31 (dd, J = 8.3, |
| | 2.1 Hz, 1H), 7.29-7.20 (m, 1H), 7.24 (d, J = 2.0 Hz, 1H), 7.14 (d, J = |
| | 7.5 Hz, 2H), 6.82-6.44 (broad s, 1H), 5.83 (s, 1H), 5.18-4.73 (broad |
| | s, 1H), 4.53 (dt, J = 13.6, 7.2 Hz, 1H), 3.34-3.24 (m, 1H), 3.23-3.07 |
| | (m, 2H), 2.32-1.89 (broad s, 6H), 1.89-1.75 (m, 1H), 1.27 (s, 9H), |
| | 1.26-1.07 (m, 2H), 0.69 (d, J = 6.3 Hz, 3H),-0.15 (d, J = 4.4 Hz, 3H) |
| 331 | ¹H NMR (499 MHz, dimethylsulfoxide-d₆) δ 13.41-11.69 (bs, 1H), |
| | 9.00 (s, 1H), 8.89 (s, 1H), 8.76 (s, 1H), 7.60 (d, J = 8.2 Hz, 1H), 7.46 |
| | (d, J = 1.9 Hz, 1H), 7.44 (s, 1H), 7.42 (dd, J = 8.1, 2.0 Hz, 1H), 7.28 (t, |
| | J = 7.6 Hz, 1H), 7.16 (d, J = 7.6 Hz, 2H), 5.53 (s, 1H), 5.49 (d, J = 18.0 |
| | Hz, 1H), 4.28 (d, J = 17.9 Hz, 1H), 3.41-3.31 (m, 1H, hidden under |
| | water peak), 2.13-1.82 (bs, 6H), 1.45-1.32 (m, 2H), 1.32 (s, 9H), 1.29- |
| | 1.18 (m, 1H), 0.57 (d, J = 5.8 Hz, 3H), 0.44 (d, J = 5.9 Hz, 3H) |
| 332 | ¹H NMR (400 MHZ, dimethylsulfoxide-d₆) δ 13.90-11.50 (bs, 1H), |
| | 8.69-8.50 (bs, 1H), 8.04-7.93 (m, 1H), 7.85-7.67 (m, 2H), 7.61 (d, |
| | J = 6.6 Hz, 1H), 7.42-7.33 (m, 3H), 7.27 (t, J = 7.5 Hz, 1H), 7.14 (d, |
| | J = 7.5 Hz, 2H), 6.72-6.44 (bs, 1H), 6.57 (d, J = 5.4 Hz, 1H), 5.23 (d, |
| | J = 18.2 Hz, 1H), 4.46-4.29 (m, 1H), 4.37 (d, J = 18.1 Hz, 1H), 2.24- |
| | 1.88 (bs, 6H), 1.32 (ddd, J = 13.8, 10.2, 3.3 Hz, 1H), 1.28-1.18 (m, |
| | 1H), 0.83 (ddd, J = 13.2, 10.1, 2.4 Hz, 1H), 0.64 (d, J = 6.6 Hz, 3H), |
| | 0.12 (d, J = 6.2 Hz, 3H) |
| 343 | ¹H NMR (400 MHZ, DMSO-d₆) δ 8.87 (d, J = 4.8 Hz, 1H), 8.71 (s, |
| | 1H), 7.80-7.66 (m, 1H), 7.59 (d, J = 7.3 Hz, 1H), 7.45-7.32 (m, 3H), |
| | 7.31-7.22 (m, 1H), 7.14 (d, J = 7.6 Hz, 2H), 6.49 (s, 1H), 6.41 (dd, J = |
| | 10.7, 4.8 Hz, 1H), 5.30 (d, J = 17.3 Hz, 1H), 4.45 (d, J = 17.2 Hz, 1H), |
| | 3.74 (dd, J = 13.3, 4.8 Hz, 1H), 3.14 (dd, J = 13.3, 10.7 Hz, 1H), 2.10 |
| | (s, 6H) |
| 344 | ¹H NMR (400 MHZ, DMSO-d₆) δ 12.18 (s, 1H), 8.76 (s, 1H), 7.93- |
| | 7.82 (m, 2H), 7.73-7.61 (m, 1H), 7.31-7.21 (m, 1H), 7.12 (d, J = 7.6 |
| | Hz, 2H), 6.29 (s, 1H), 5.72-5.55 (m, 1H), 4.73 (t, J = 10.8 Hz, 1H), |
| | 4.55 (d, J = 11.9 Hz, 1H), 3.44 (d, J = 5.7 Hz, 2H), 3.40-3.29 (m, 1H), |
| | 3.19-3.06 (m, 3H), 3.05-2.91 (m, 1H), 2.70-2.58 (m, 1H), 2.43 (d, J = |
| | 12.4 Hz, 1H), 2.26 (d, J = 11.9 Hz, 1H), 2.05 (s, 6H), 1.66 (q, J = |
| | 11.9 Hz, 1H), 0.92 (s, 9H) |
| 350 | ¹H NMR (400 MHZ, DMSO-d₆) δ 7.39 (d, J = 1.8 Hz, 1H), 7.20 (dd, |
| | J = 8.1, 7.0 Hz, 1H), 7.16-7.06 (m, 2H), 6.45 (d, J = 1.8 Hz, 1H), 6.29 |
| | (s, 1H), 5.48-5.33 (m, 1H), 4.55 (d, J = 12.9 Hz, 1H), 3.90 (d, J = 16.0 |
| | Hz, 1H), 3.70-3.60 (m, 1H), 3.54 (s, 3H), 3.49 (d, J = 15.7 Hz, 1H), |
| | 2.97-2.86 (m, 1H), 2.58-2.52 (m, 1H), 2.11 (s, 1H), 2.01 (s, 6H), 1.94- |
| | 1.83 (m, 1H), 1.78-1.63 (m, 1H), 1.60-1.41 (m, 1H) |
| 355 | ¹H NMR (400 MHZ, DMSO-d₆) δ 8.04 (t, J = 1.8 Hz, 1H), 7.80 (dt, |
| | J = 6.9, 1.9 Hz, 1H), 7.69-7.54 (m, 2H), 7.31-7.21 (m, 1H), 7.13 (d, J = |
| | 7.6 Hz, 2H), 6.35 (s, 1H), 4.40 (s, 1H), 4.14 (td, J = 10.0, 6.9 Hz, |
| | 1H), 3.94 (td, J = 10.0, 4.7 Hz, 1H), 3.75 (ddd, J = 11.8, 8.3, 6.1 Hz, |
| | 1H), 3.49 (dd, J = 11.1, 5.2 Hz, 1H), 3.31 (ddd, J = 12.0, 8.6, 5.5 Hz, |
| | 1H), 3.13 (dd, J = 11.2, 3.9 Hz, 1H), 2.05 (s, 7H), 1.83-1.73 (m, 1H), |
| | 1.66-1.53 (m, 2H), 1.41 (dd, J = 15.4, 6.1 Hz, 1H), 1.05 (dq, J = 17.3, |
| | 11.4, 9.2 Hz, 1H). |
| 356 | ¹H NMR (400 MHZ, DMSO-d₆) δ 12.48 (d, J = 493.5 Hz, 1H), 8.04 (s, |
| | 1H), 7.80 (s, 1H), 7.69-7.50 (m, 2H), 7.23 (d, J = 7.8 Hz, 1H), 7.12 |
| | (d, J = 7.6 Hz, 2H), 6.41 (s, 1H), 4.28 (s, 1H), 4.07 (dt, J = 13.8, 6.6 |
| | Hz, 1H), 3.53 (t, J = 8.6 Hz, 1H), 3.25 (t, J = 10.0 Hz, 1H), 2.15 (s, |
| | 1H), 2.05 (d, J = 22.3 Hz, 6H), 1.94 (dd, J = 12.2, 6.1 Hz, 1H), 1.90- |
| | 1.82 (m, 1H), 1.78 (dd, J = 12.8, 9.8 Hz, 1H), 1.63 (d, J = 11.6 Hz, 1H), |
| | 1.59 (s, 3H), 1.54 (d, J = 12.8 Hz, 1H), 1.48 (s, 3H), 1.30-1.20 (m, 1H). |
| 357 | ¹H NMR (400 MHZ, DMSO-d₆) δ 10.65 (d, J = 101.7 Hz, 1H), 8.59 (d, |
| | J = 39.5 Hz, 1H), 7.95 (s, 1H), 7.67 (dd, J = 13.5, 5.9 Hz, 2H), 7.29 (s, |
| | 1H), 7.15 (s, 2H), 6.27 (d, J = 98.0 Hz, 1H), 5.80 (s, 1H), 3.95 (d, J = |
| | 98.6 Hz, 12H), 3.48-3.17 (m, 6H), 2.10 (d, J = 42.1 Hz, 8H), 1.85 (s, |
| | 2H), 1.49 (s, 2H), 0.87 (d, J = 55.8 Hz, 4H). |
| 358 | ¹H NMR (400 MHZ, DMSO-d₆) δ 13.05 (s, 1H), 10.23 (s, 1H), 8.61 (s, |
| | 1H), 7.94 (s, 1H), 7.69 (s, 2H), 7.28 (s, 1H), 7.14 (d, J = 7.5 Hz, 2H), |
| | 6.35 (s, 1H), 5.77 (s, 1H), 4.39 (s, 1H), 4.03 (s, 1H), 3.90-3.74 (m, |
| | 2H), 2.19-1.96 (m, 10H), 0.97 (d, J = 23.4 Hz, 4H). |
| 359 | ¹H NMR (400 MHZ, DMSO-d₆) δ 8.31 (s, 1H), 7.88 (s, 1H), 7.63 (d, J = |
| | 8.0 Hz, 2H), 7.24 (s, 1H), 7.10 (s, 2H), 6.27 (s, 1H), 5.74 (s, 1H), 4.45 |
| | (s, 1H), 4.06 (d, J = 14.0 Hz, 1H), 3.90 (d, J = 13.8 Hz, 1H), 3.70-3.41 |
| | (m, 2H), 3.17 (s, 1H), 3.03 (d, J = 43.8 Hz, 1H), 2.01 (s, 6H), 1.80 (q, |
| | J = 13.0, 12.2 Hz, 1H), 1.71-1.56 (m, 1H), 1.42 (d, J = 9.8 Hz, 9H). |

### Example 142: Compounds 372 to 385

Compounds 372 to 385, depicted in the following table, can be prepared following the procedures described above for compounds 1-371 and CFTR modulating activity can be assessed using one or more of the assays outlined below.

TABLE 12

| Compound Number | Structure |
|---|---|
| 372 | 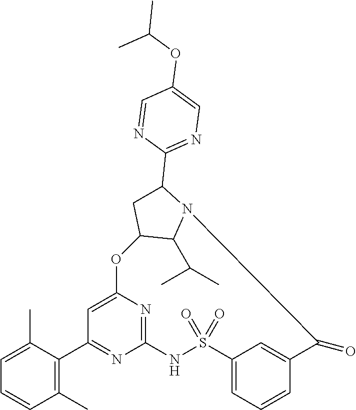 [SMILES: Cc1cccc(C)c1-c1cc2nc(n1)NS(=O)(=O)c1cccc(c1)C(=O)N1C(c3ncc(OC(C)C)cn3)CC(O2)C1C(C)C] |
| 373 | 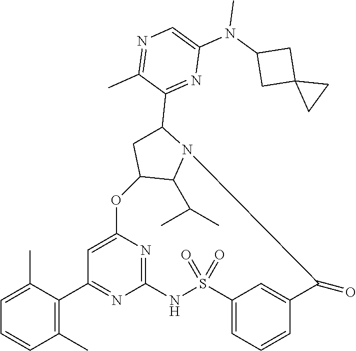 [SMILES: Cc1cccc(C)c1-c1cc2nc(n1)NS(=O)(=O)c1cccc(c1)C(=O)N1C(c3nc(N(C)C4CC5(CC5)C4)cnc3C)CC(O2)C1C(C)C] |
| 374 | 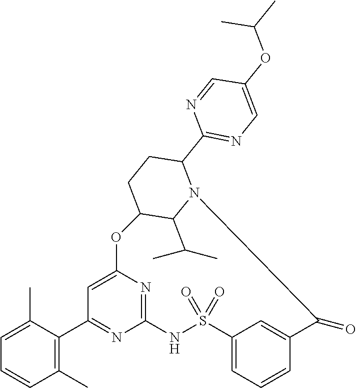 [SMILES: Cc1cccc(C)c1-c1cc2nc(n1)NS(=O)(=O)c1cccc(c1)C(=O)N1C(c3ncc(OC(C)C)cn3)CCC(O2)C1C(C)C] |
| 375 | 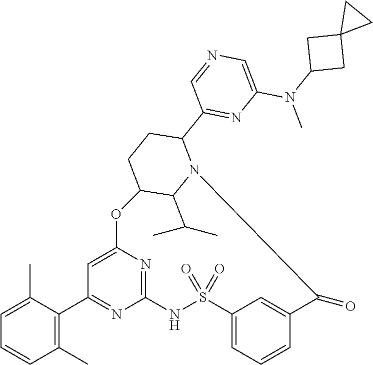 [SMILES: Cc1cccc(C)c1-c1cc2nc(n1)NS(=O)(=O)c1cccc(c1)C(=O)N1C(c3cncc(N(C)C4CC5(CC5)C4)n3)CCC(O2)C1C(C)C] |
| 376 | 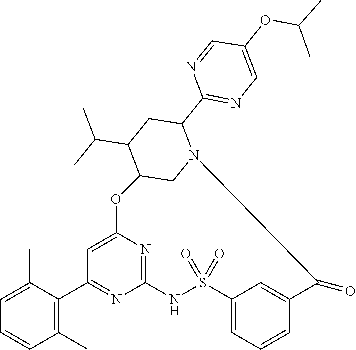 [SMILES: Cc1cccc(C)c1-c1cc2nc(n1)NS(=O)(=O)c1cccc(c1)C(=O)N1CC(O2)C(C(C)C)CC1c1ncc(OC(C)C)cn1] |
| 377 | 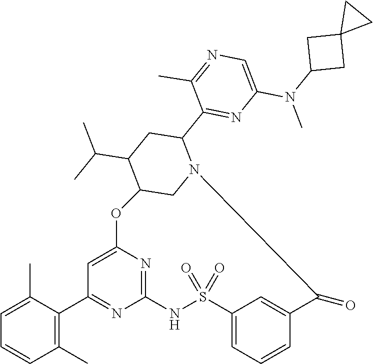 [SMILES: Cc1cccc(C)c1-c1cc2nc(n1)NS(=O)(=O)c1cccc(c1)C(=O)N1CC(O2)C(C(C)C)CC1c1nc(N(C)C2CC3(CC3)C2)cnc1C] |
| 378 | 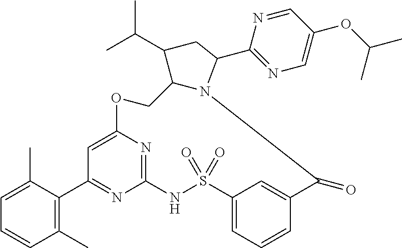 [SMILES: Cc1cccc(C)c1-c1cc2nc(n1)NS(=O)(=O)c1cccc(c1)C(=O)N1C(c3ncc(OC(C)C)cn3)CC(C(C)C)C1CO2] |
| 379 | 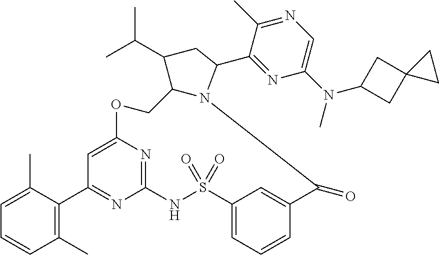 [SMILES: Cc1cccc(C)c1-c1cc2nc(n1)NS(=O)(=O)c1cccc(c1)C(=O)N1C(c3nc(N(C)C4CC5(CC5)C4)cnc3C)CC(C(C)C)C1CO2] |
| 380 | 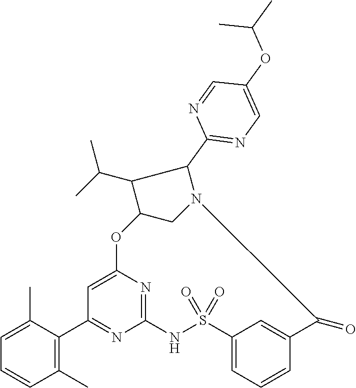 [SMILES: Cc1cccc(C)c1-c1cc2nc(n1)NS(=O)(=O)c1cccc(c1)C(=O)N1CC(O2)C(C(C)C)C1c1ncc(OC(C)C)cn1] |
| 381 | 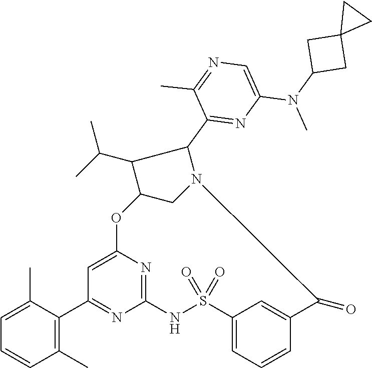 [SMILES: Cc1cccc(C)c1-c1cc2nc(n1)NS(=O)(=O)c1cccc(c1)C(=O)N1CC(O2)C(C(C)C)C1c1nc(N(C)C2CC3(CC3)C2)cnc1C] |
| 382 | 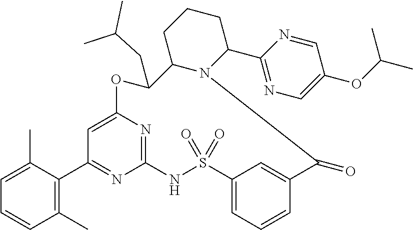 [SMILES: Cc1cccc(C)c1-c1cc2nc(n1)NS(=O)(=O)c1cccc(c1)C(=O)N1C(c3ncc(OC(C)C)cn3)CCCC1C(CC(C)C)O2] |
| 383 | 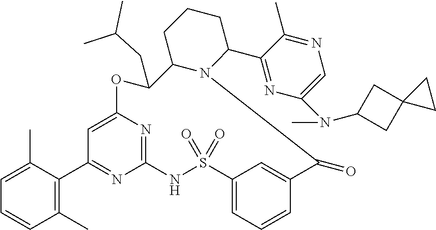 [SMILES: Cc1cccc(C)c1-c1cc2nc(n1)NS(=O)(=O)c1cccc(c1)C(=O)N1C(c3nc(N(C)C4CC5(CC5)C4)cnc3C)CCCC1C(CC(C)C)O2] |
| 384 | 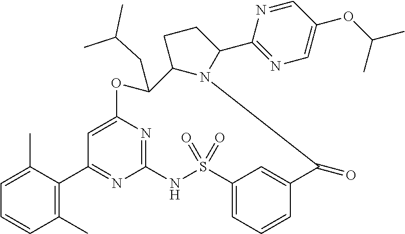 [SMILES: Cc1cccc(C)c1-c1cc2nc(n1)NS(=O)(=O)c1cccc(c1)C(=O)N1C(c3ncc(OC(C)C)cn3)CCC1C(CC(C)C)O2] |
| 385 | 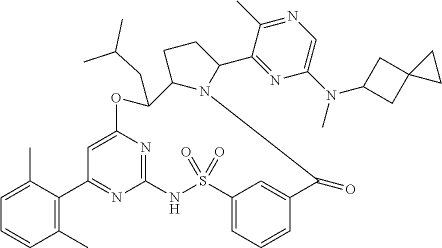 [SMILES: Cc1cccc(C)c1-c1cc2nc(n1)NS(=O)(=O)c1cccc(c1)C(=O)N1C(c3nc(N(C)C4CC5(CC5)C4)cnc3C)CCC1C(CC(C)C)O2] |

### VI. Biological Activity of Compounds

A. 3t3 Assay

1. Membrane Potential Optical Methods for Assaying F508del Modulation Properties of Compounds

The assay utilizes fluorescent voltage sensing dyes to measure changes in membrane potential using a fluorescent plate reader (e.g., FLTPR III, Molecular Devices, Inc.) as a readout for increase in functional F508del in NIH 3T3 cells. The driving force for the response is the creation of a chloride ion gradient in conjunction with channel activation by a single liquid addition step after the cells have previously been treated with compounds and subsequently loaded with a voltage sensing dye.

2. Identification of Corrector Compounds

To identify correctors of F508del, a single-addition HTS assay format was developed. This HTS assay utilizes fluorescent voltage sensing dyes to measure changes in membrane potential on the FLIPR III as a measurement for increase in gating (conductance) of F508del in F508del NIH 3T3 cells. The F508del NIH 3T3 cell cultures were incubated with the corrector compounds at a range of concentrations for 18-24 hours at 37° C., and subsequently loaded with a redistribution dye. The driving force for the response is a Cl⁻ ion gradient in conjunction with channel activation with forskolin in a single liquid addition step using a fluorescent plate reader such as FLIPR III. The efficacy and potency of the putative F508del correctors was compared to that of the known corrector, lumacaftor, in combination with acutely added 300 nM Ivacaftor.

3. Solutions

Bath Solution #1: (in mM) NaCl 160, KCl 4.5, CaCl₂ 2, MgCl₂ 1, HEPES 10, pH 7.4 with NaOH.

Chloride-free bath solution: Chloride salts in Bath Solution #1 (above) are substituted with gluconate salts.

4. Cell Culture

NIH3T3 mouse fibroblasts stably expressing F508del are used for optical measurements of membrane potential. The cells are maintained at 37° C. in 5% CO₂ and 90% humidity in Dulbecco's modified Eagle's medium supplemented with 2 mM glutamine, 10% fetal bovine serum, 1×NEAA, b-ME, 1× pen/strep, and 25 mM HEPES in 175 cm² culture flasks. For all optical assays, the cells were seeded at ˜20,000/well in 384-well Matrigel-coated plates. For the correction assays, the cells are cultured at 37° C. with and without compounds for 16-24 hours.

B. Enteroid Assay

1. Solutions

Base medium (ADF+++) consisted of Advanced DMEM/Ham's F12, 2 mM Glutamax, 10 mM HEPES, 1 μg/mL penicillin/streptomycin.

Intestinal enteroid maintenance medium (IEMM) consisted of ADF+++, 1× B27 supplement, 1× N2 supplement, 1.25 mM N-acetyl cysteine, 10 mM Nicotinamide, 50 ng/mL hEGF, 10 nM Gastrin, 1 μg/mL hR-spondin-1, 100 ng/mL hNoggin, TGF-b type 1 inhibitor A-83-01, 100 μg/mL Primocin, 10 μM P38 MAPK inhibitor SB202190.

Bath 1 Buffer consisted of 1 mM MgCl₂, 160 mM NaCl, 4.5 mM KCl, 10 mM HEPES, 10 mM Glucose, 2 mM CaCl₂.

Chloride Free Buffer consisted of 1 mM Magnesium Gluconate, 2 mM Calcium Gluconate, 4.5 mM Potassium Gluconate, 160 mM Sodium Gluconate, 10 mM HEPES, 10 mM Glucose.

Bath1 Dye Solution consisted of Bath 1 Buffer, 0.04% Pluronic F127, 20 μM Methyl Oxonol, 30 μM CaCCinh-A01, 30 μM Chicago Sky Blue.

Chloride Free Dye Solution consisted of Chloride Free Buffer, 0.04% Pluronic F127, 20 μM Methyl Oxonol, 30 μM CaCCinh-A01, 30 μM Chicago Sky Blue.

Chloride Free Dye Stimulation Solution consisted of Chloride Free Dye Solution, 10 μM forskolin, 100 μM IBMX, and 300 nM Compound III.

2. Cell Culture

Human intestinal epithelial enteroid cells were obtained from the Hubrecht Institute for Developmental Biology and Stem Cell Research, Utrecht, The Netherlands and expanded in T-Flasks as previously described (Dekkers J F, Wiegerinck C L, de Jonge H R, Bronsveld I, Janssens H M, de Winter-de Groot K M, Brandsma A M, de Jong N W M, Bijvelds M J C, Scholte B J, Nieuwenhuis E E S, van den Brink S, Clevers H, van der Ent C K, Middendorp S and M Beekman J M. A functional CFTR assay using primary cystic fibrosis intestinal organoids. Nat Med. 2013 July; 19(7):939-45.).

3. Enteroid Cell Harvesting and Seeding

Cells were recovered in cell recovery solution, collected by centrifugation at 650 rpm for 5 min at 4° C., resuspended in TryPLE and incubated for 5 min at 37° C. Cells were then collected by centrifugation at 650 rpm for 5 min at 4° C. and resuspended in IEMM containing 10 μM ROCK inhibitor (RI). The cell suspension was passed through a 40 μm cell strainer and resuspended at 1×106 cells/mL in IEMM containing 10 μM RI. Cells were seeded at 5000 cells/well into multi-well plates and incubated for overnight at 37° C., 95% humidity and 5% CO₂ prior to assay.

4. Membrane Potential Dye, Enteroid Assay A

Enteroid cells were incubated with test compound in IEMM for 18-24 hours at 37° C., 95% humidity and 5% CO₂. Following compound incubations, a membrane potential dye assay was employed using a FLIPR Tetra to directly measure the potency and efficacy of the test compound on CFTR-mediated chloride transport following acute addition of 10 μM forskolin and 300 nM N-[2,4-bis(1,1-dimethylethyl)-5-hydroxyphenyl]-1,4-dihydro-4-oxoquinoline-3-carboxamide. Briefly, cells were washed 5 times in Bath 1 Buffer. Bath 1 Dye Solution was added, and the cells were incubated for 25 min at room temperature. Following dye incubation, cells were washed 3 times in Chloride Free Dye Solution. Chloride transport was initiated by addition of Chloride Free Dye Stimulation Solution and the fluorescence signal was read for 15 min. The CFTR-mediated chloride transport for each condition was determined from the AUC of the fluorescence response to acute forskolin and 300 nM N-[2,4-bis(1,1-dimethylethyl)-5-hydroxyphenyl]-1,4-dihydro-4-oxoquinoline-3-carboxamide stimulation. Chloride transport was then expressed as a percentage of the chloride transport following treatment with 3 μM N-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide, 3 μM (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide and 300 nM acute N-[2,4-bis(1,1-dimethylethyl)-5-hydroxyphenyl]-1,4-dihydro-4-oxoquinoline-3-carboxamide triple combination control (% Activity).

5. Membrane Potential Dye, Enteroid Assay B

Enteroid cells were incubated with test compound in IEMM for 18-24 hours at 37° C., 95% humidity and 5% CO₂. Following compound incubations, a membrane potential dye assay was employed using a FLIPR Tetra to directly measure the potency and efficacy of the test compound on CFTR-mediated chloride transport following acute addition of 10 μM forskolin and 300 nM N-[2,4-bis(1,1-dimethylethyl)-5-hydroxyphenyl]-1,4-dihydro-4-oxoquinoline-3-carboxamide. Briefly, cells were washed 5 times in Bath 1 Buffer. Bath 1 Dye Solution was added and the cells were incubated for 25 min at room temperature. Following dye incubation, cells were washed 3 times in Chloride Free Dye Solution. Chloride transport was initiated by addition of Chloride Free Dye Stimulation Solution and the fluorescence signal was read for 15 min. The CFTR-mediated chloride transport for each condition was determined from the AUC of the fluorescence response to acute forskolin and 300 nM N-[2,4-bis(1,1-dimethylethyl)-5-hydroxyphenyl]-1,4-dihydro-4-oxoquinoline-3-carboxamide stimulation. Chloride transport was then expressed as a percentage of the chloride transport following treatment with 1 μM (14S)-8-[3-(2-{Dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, 3 μM (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide and 300 nM acute N-[2,4-bis(1,1-dimethylethyl)-5-hydroxyphenyl]-1,4-dihydro-4-oxoquinoline-3-carboxamide triple combination control (% Activity).

C. HBE Assay

1. Ussing Chamber Assay of CFTR-Mediated Short-Circuit Currents

Ussing chamber experiments were performed using human bronchial epithelial (HBE) cells derived from CF subjects heterozygous for F508del and a minimal function CFTR mutation (F508del/MF-HBE) and cultured as previously described (Neuberger T, Burton B, Clark H, Van Goor F Methods Mol Biol 2011:741:39-54). After four days the apical media was removed, and the cells were grown at an air liquid interface for >14 days prior to use. This resulted in a monolayer of fully differentiated columnar cells that were ciliated, features that are characteristic of human bronchial airway epithelia.

To isolate the CFTR-mediated short-circuit (I_{SC}) current, F508del/MF-HBE grown on Costar® Snapwell™ cell culture inserts were mounted in an Ussing chamber and the transepithelial I_{SC} was measured under voltage-clamp recording conditions (V_{hold}=0 mV) at 37° C. The basolateral solution contained (in mM) 145 NaCl, 0.83 K₂HPO₄, 3.3 KH₂PO₄, 1.2 MgCl₂, 1.2 CaCl₂), 10 Glucose, 10 HEPES (pH adjusted to 7.4 with NaOH) and the apical solution contained (in mM) 145 NaGluconate, 1.2 MgCl₂, 1.2 CaCl₂, 10 glucose, 10 HEPES (pH adjusted to 7.4 with NaOH) and 30 μM amiloride to block the epithelial sodium channel. Forskolin (20 μM) was added to the apical surface to activate CFTR, followed by apical addition of a CFTR inhibitor cocktail consisting of BPO, GlyH-101, and CFTR inhibitor 172 (each at 20 μM final assay concentration) to specifically isolate CFTR currents. The CFTR-mediated I_{SC} (μA/cm²) for each condition was determined from the peak forskolin response to the steady-state current following inhibition.

2. Identification of Corrector Compounds

The activity of the CFTR corrector compounds on the CFTR-mediated I_{SC} was determined in Ussing chamber studies as described above. The F508del/MF-HBE cell cultures were either incubated with the corrector compounds at a range of concentrations in combination with 1 μM Ivacaftor or were incubated with the corrector compounds at a single fixed concentration of 10 μM in combination with 1 μM Ivacaftor for 18-24 hours at 37° C. and in the presence of 20% human serum. The concentration of corrector compounds with 1 μM Ivacaftor during the 18-24 hours incubations was kept constant throughout the Ussing chamber measurement of the CFTR-mediated I_{SC} to ensure compounds were present throughout the entire experiment. The efficacy and potency of the putative F508del correctors was compared to that of the known Vertex corrector, (14S)-8-[3-(2-{Dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-tri one, in combination with 18 M Tezacaftor and 1 μM Ivacaftor.

D. Biological Activity Data Tables

The following table represent CFTR modulating activity for representative compounds of the disclosure generated using one or more of the assays disclosed herein (EC₅₀: +++ is <1 μM; ++ is 1-<3 μM; + is 3-<30 μM; and ND is “not detected in this assay.” 0 Activity: +++ is >60%; ++ is 30-60%; + is <300%).

TABLE 13

| | | | Ent. A | | Ent. B |
|---|---|---|---|---|---|
| | | Ent. A | Max | Ent. B | Max |
| Cmpd | | EC₅₀ | Activity | EC₅₀ | Activity |
| Number | Structure | (μM) | (%) | (μM) | (%) |
| 334 | 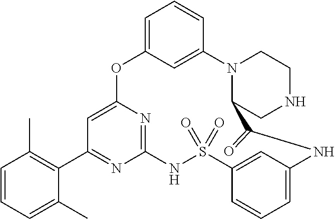 [SMILES: Cc1cccc(C)c1-c1cc2nc(n1)NS(=O)(=O)c1cccc(c1)NC(=O)[C@@H]1CNCCN1c1cccc(c1)O2] | ++ | +++ | | |
| 252 | 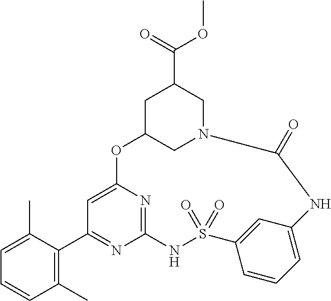 [SMILES: COC(=O)C1CC2CN(C1)C(=O)Nc1cccc(c1)S(=O)(=O)Nc1nc(cc(-c3c(C)cccc3C)n1)O2] | ++ | ++ | | |
| 326 | 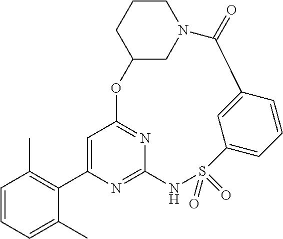 [SMILES: Cc1cccc(C)c1-c1cc2nc(n1)NS(=O)(=O)c1cccc(c1)C(=O)N1CCCC(C1)O2] | + | + | | |
| 324 | 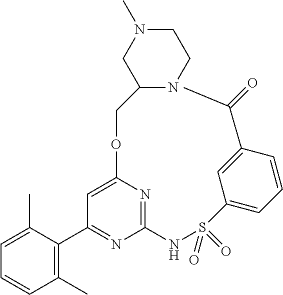 [SMILES: CC(=O)c1cccc(S(=O)(=O)Nc2nc(OCC3CN(C)CCN3)cc(-c3c(C)cccc3C)n2)c1] | + | ++ | | |
| 354 | 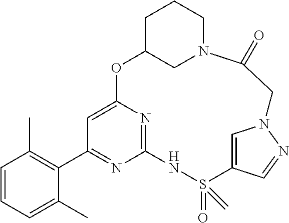 [SMILES: C=S1(=O)Nc2nc(cc(-c3c(C)cccc3C)n2)OC2CCCN(C2)C(=O)Cn2cc1cn2] | ND | + | | |
| 171 | 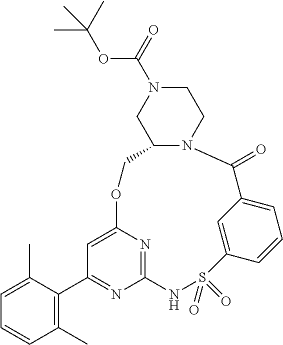 [SMILES: Cc1cccc(C)c1-c1cc2nc(n1)NS(=O)(=O)c1cccc(c1)C(=O)N1CCN(C(=O)OC(C)(C)C)C[C@H]1CO2] | ND | + | | |
| 323 | 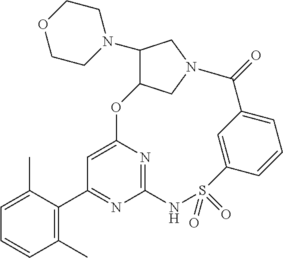 [SMILES: Cc1cccc(C)c1-c1cc2nc(n1)NS(=O)(=O)c1cccc(c1)C(=O)N1CC(O2)C(N2CCOCC2)C1] | + | ++ | | |
| 353 | 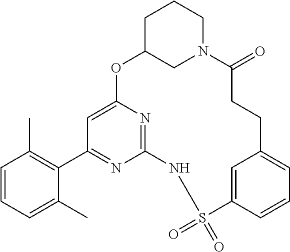 [SMILES: Cc1cccc(C)c1-c1cc2nc(n1)NS(=O)(=O)c1cccc(c1)CCC(=O)N1CCCC(C1)O2] | ND | + | | |
| 352 | 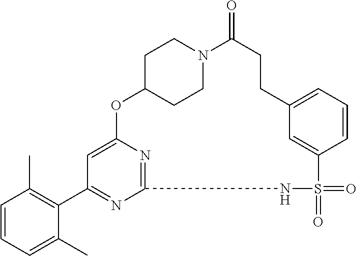 | ND | + | | |
| 351 | 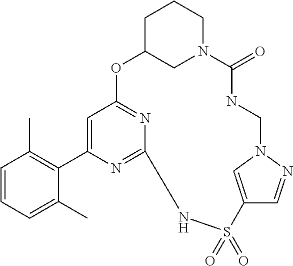 [SMILES: Cc1cccc(C)c1-c1cc2nc(n1)NS(=O)(=O)c1cnn(c1)CNC(=O)N1CCCC(C1)O2] | ND | + | | |
| 350 | 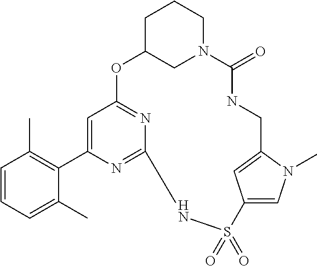 [SMILES: Cc1cccc(C)c1-c1cc2nc(n1)NS(=O)(=O)c1cc(n(C)c1)CNC(=O)N1CCCC(C1)O2] | ND | + | | |
| 160 | 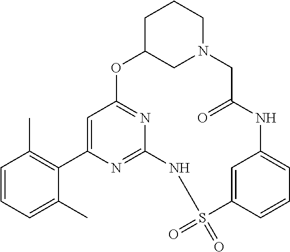 [SMILES: Cc1cccc(C)c1-c1cc2nc(n1)NS(=O)(=O)c1cccc(c1)NC(=O)CN1CCCC(C1)O2] | ++ | + | | |
| 349 | 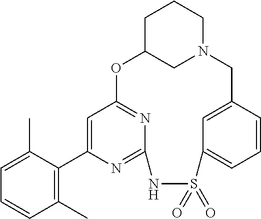 [SMILES: Cc1cccc(C)c1-c1cc2nc(n1)NS(=O)(=O)c1cccc(c1)CN1CCCC(C1)O2] | ND | + | | |
| 320 | 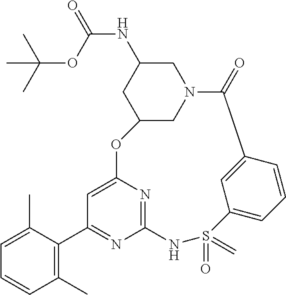 [SMILES: C=S1(=O)Nc2nc(cc(-c3c(C)cccc3C)n2)OC2CC(NC(=O)OC(C)(C)C)CN(C2)C(=O)c2cccc1c2] | ++ | ++ | | |
| 321 | 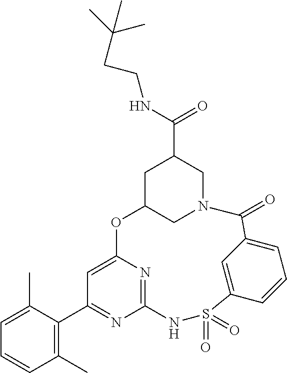 [SMILES: Cc1cccc(C)c1-c1cc2nc(n1)NS(=O)(=O)c1cccc(c1)C(=O)N1CC(CC(C(=O)NCCC(C)(C)C)C1)O2] | +++ | ++ | | |
| 322 | 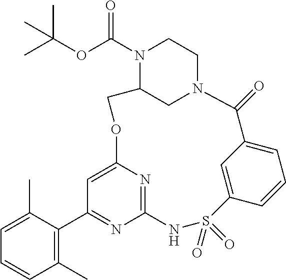 [SMILES: Cc1cccc(C)c1-c1cc2nc(n1)NS(=O)(=O)c1cccc(c1)C(=O)N1CCN(C(=O)OC(C)(C)C)C(CO2)C1] | +++ | ++ | | |
| 325 | 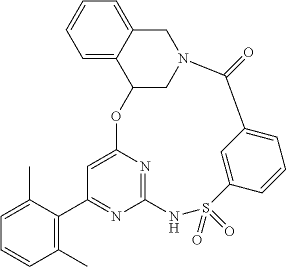 [SMILES: Cc1cccc(C)c1-c1cc2nc(n1)NS(=O)(=O)c1cccc(c1)C(=O)N1Cc3ccccc3C(C1)O2] | +++ | +++ | | |
| 212 | 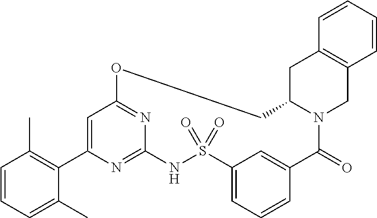 [SMILES: Cc1cccc(C)c1-c1cc2nc(n1)NS(=O)(=O)c1cccc(c1)C(=O)N1Cc3ccccc3C[C@H]1CO2] | +++ | ++ | | |
| 211 | 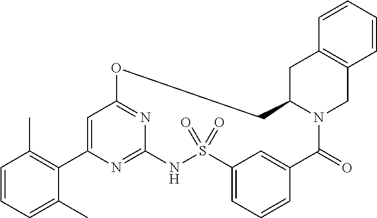 [SMILES: Cc1cccc(C)c1-c1cc2nc(n1)NS(=O)(=O)c1cccc(c1)C(=O)N1Cc3ccccc3C[C@@H]1CO2] | +++ | +++ | | |
| 208 | 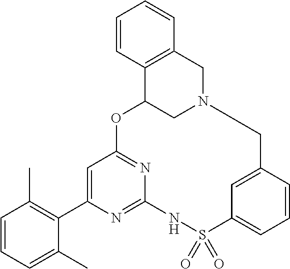 [SMILES: Cc1cccc(C)c1-c1cc2nc(n1)NS(=O)(=O)c1cccc(c1)CN1Cc3ccccc3C(C1)O2] | +++ | ++ | | |
| 348 | 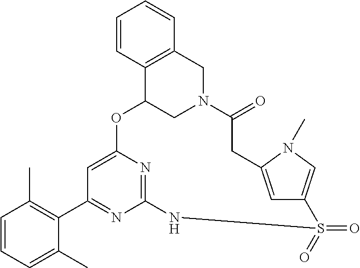 [SMILES: Cc1cccc(C)c1-c1cc2nc(n1)NS(=O)(=O)c1cc(n(C)c1)CC(=O)N1Cc3ccccc3C(C1)O2] | ++ | ++ | | |
| 347 | 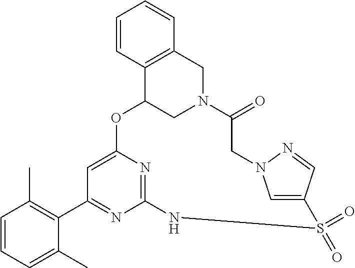 [SMILES: Cc1cccc(C)c1-c1cc2nc(n1)NS(=O)(=O)c1cnn(c1)CC(=O)N1Cc3ccccc3C(C1)O2] | ND | + | | |
| 346 | 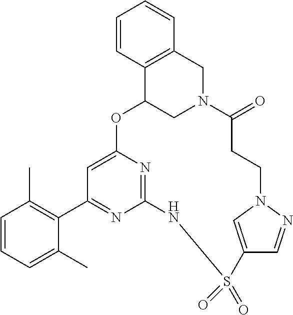 [SMILES: Cc1cccc(C)c1-c1cc2nc(n1)NS(=O)(=O)c1cnn(c1)CCC(=O)N1Cc3ccccc3C(C1)O2] | ND | + | | |
| 345 | 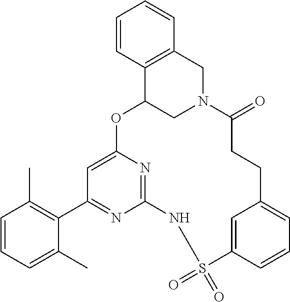 [SMILES: Cc1cccc(C)c1-c1cc2nc(n1)NS(=O)(=O)c1cccc(c1)CCC(=O)N1Cc3ccccc3C(C1)O2] | ND | + | | |
| 344 | 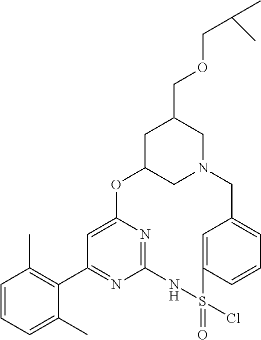 | +++ | ++ | | |
| 318 | 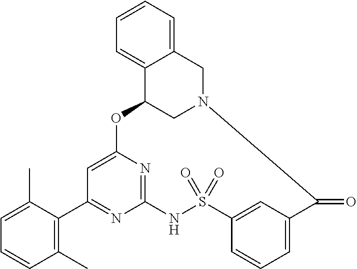 [SMILES: Cc1cccc(C)c1-c1cc2nc(n1)NS(=O)(=O)c1cccc(c1)C(=O)N1Cc3ccccc3[C@@H](C1)O2] | ND | + | | |
| 319 | 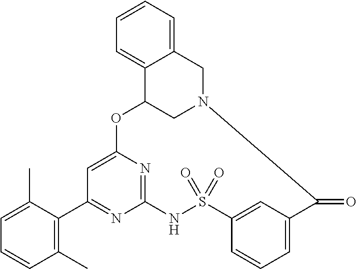 [SMILES: Cc1cccc(C)c1-c1cc2nc(n1)NS(=O)(=O)c1cccc(c1)C(=O)N1Cc3ccccc3C(C1)O2] | +++ | +++ | +++ | +++ |
| 317 | 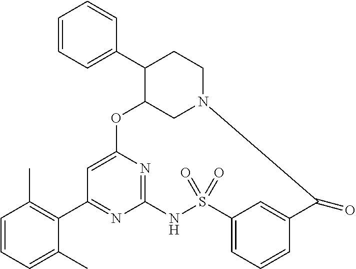 [SMILES: Cc1cccc(C)c1-c1cc2nc(n1)NS(=O)(=O)c1cccc(c1)C(=O)N1CCC(c3ccccc3)C(C1)O2] | +++ | +++ | | |
| 370 | 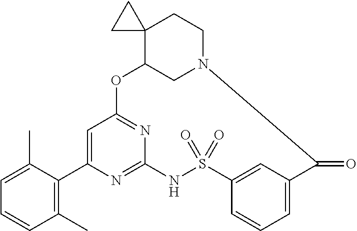 [SMILES: Cc1cccc(C)c1-c1cc2nc(n1)NS(=O)(=O)c1cccc(c1)C(=O)N1CCC3(CC3)C(C1)O2] | +++ | +++ | | |
| 315 | 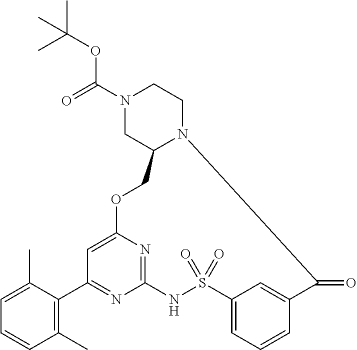 [SMILES: Cc1cccc(C)c1-c1cc2nc(n1)NS(=O)(=O)c1cccc(c1)C(=O)N1CCN(C(=O)OC(C)(C)C)C[C@@H]1CO2] | +++ | ++ | | |
| 316 | 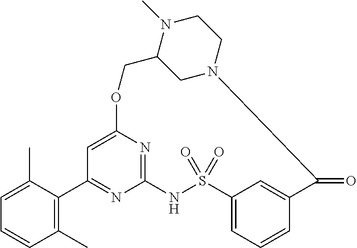 [SMILES: Cc1cccc(C)c1-c1cc2nc(n1)NS(=O)(=O)c1cccc(c1)C(=O)N1CCN(C)C(CO2)C1] | ND | + | | |
| 207 | 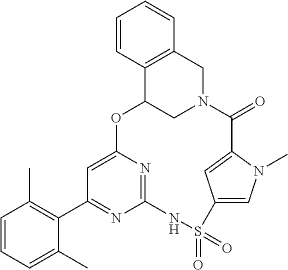 [SMILES: Cc1cccc(C)c1-c1cc2nc(n1)NS(=O)(=O)c1cc(n(C)c1)C(=O)N1Cc3ccccc3C(C1)O2] | +++ | ++ | | |
| 343 | 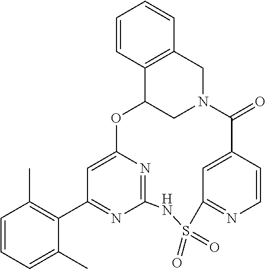 [SMILES: Cc1cccc(C)c1-c1cc2nc(n1)NS(=O)(=O)c1cc(ccn1)C(=O)N1Cc3ccccc3C(C1)O2] | ++ | ++ | | |
| 170 | 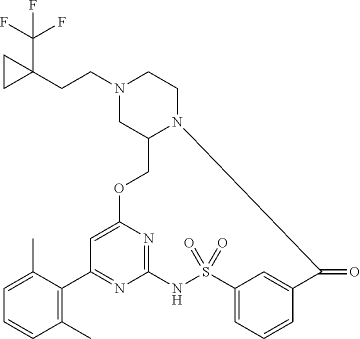 [SMILES: Cc1cccc(C)c1-c1cc2nc(n1)NS(=O)(=O)c1cccc(c1)C(=O)N1CCN(CCC3(C(F)(F)F)CC3)CC1CO2] | +++ | ++ | | |
| 314 | 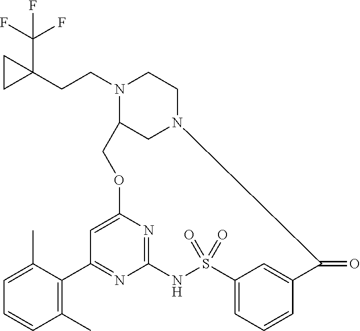 [SMILES: Cc1cccc(C)c1-c1cc2nc(n1)NS(=O)(=O)c1cccc(c1)C(=O)N1CCN(CCC3(C(F)(F)F)CC3)C(CO2)C1] | +++ | +++ | | |
| 342 | 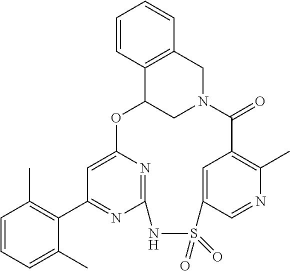 [SMILES: Cc1cccc(C)c1-c1cc2nc(n1)NS(=O)(=O)c1cnc(C)c(c1)C(=O)N1Cc3ccccc3C(C1)O2] | ++ | + | | |
| 178 | 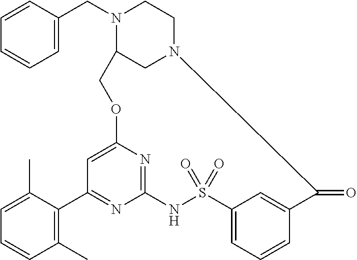 [SMILES: Cc1cccc(C)c1-c1cc2nc(n1)NS(=O)(=O)c1cccc(c1)C(=O)N1CCN(Cc3ccccc3)C(CO2)C1] | +++ | ++ | | |
| 313 | 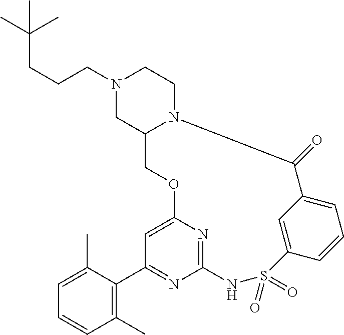 [SMILES: Cc1cccc(C)c1-c1cc2nc(n1)NS(=O)(=O)c1cccc(c1)C(=O)N1CCN(CCCC(C)(C)C)CC1CO2] | +++ | +++ | | |
| 312 | 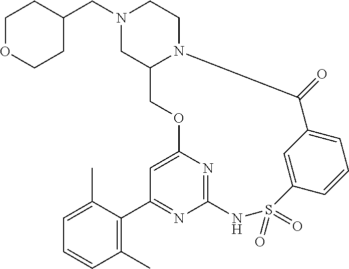 [SMILES: Cc1cccc(C)c1-c1cc2nc(n1)NS(=O)(=O)c1cccc(c1)C(=O)N1CCN(CC3CCOCC3)CC1CO2] | ++ | ++ | | |
| 311 | 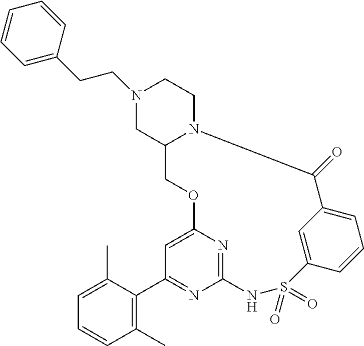 [SMILES: Cc1cccc(C)c1-c1cc2nc(n1)NS(=O)(=O)c1cccc(c1)C(=O)N1CCN(CCc3ccccc3)CC1CO2] | +++ | ++ | | |
| 310 | 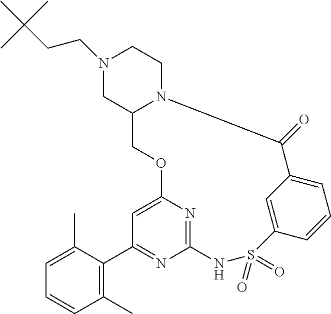 [SMILES: Cc1cccc(C)c1-c1cc2nc(n1)NS(=O)(=O)c1cccc(c1)C(=O)N1CCN(CCC(C)(C)C)CC1CO2] | +++ | ++ | | |
| 169 | 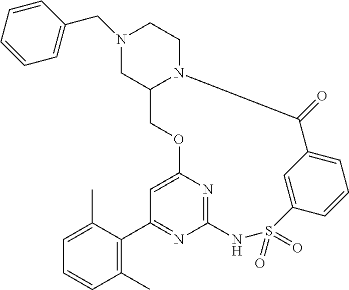 [SMILES: Cc1cccc(C)c1-c1cc2nc(n1)NS(=O)(=O)c1cccc(c1)C(=O)N1CCN(Cc3ccccc3)CC1CO2] | +++ | +++ | | |
| 177 | 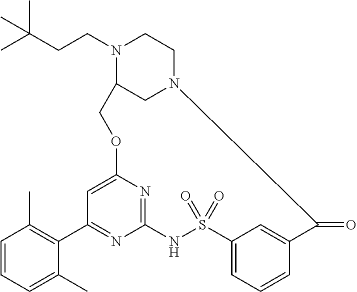 [SMILES: Cc1cccc(C)c1-c1cc2nc(n1)NS(=O)(=O)c1cccc(c1)C(=O)N1CCN(CCC(C)(C)C)C(CO2)C1] | +++ | ++ | | |
| 309 | 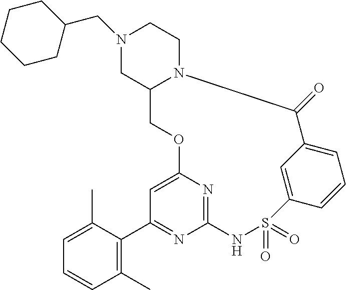 [SMILES: Cc1cccc(C)c1-c1cc2nc(n1)NS(=O)(=O)c1cccc(c1)C(=O)N1CCN(CC3CCCCC3)CC1CO2] | +++ | ++ | | |
| 308 | 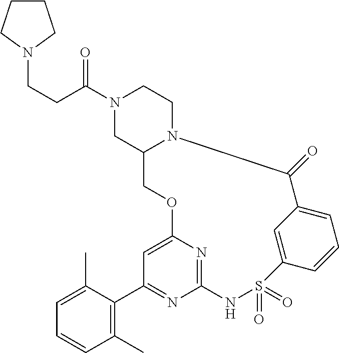 [SMILES: Cc1cccc(C)c1-c1cc2nc(n1)NS(=O)(=O)c1cccc(c1)C(=O)N1CCN(C(=O)CCN3CCCC3)CC1CO2] | ND | + | | |
| 307 | 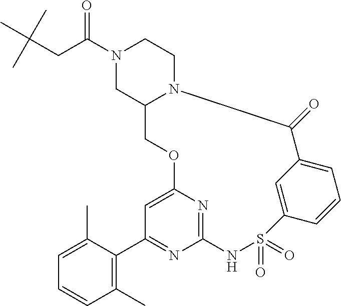 [SMILES: Cc1cccc(C)c1-c1cc2nc(n1)NS(=O)(=O)c1cccc(c1)C(=O)N1CCN(C(=O)CC(C)(C)C)CC1CO2] | + | ++ | | |
| 306 | 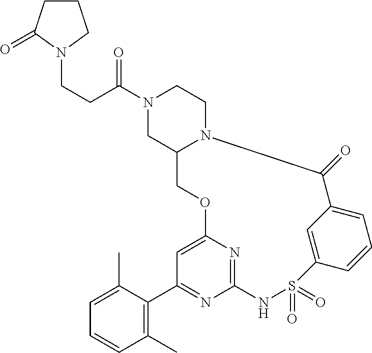 [SMILES: Cc1cccc(C)c1-c1cc2nc(n1)NS(=O)(=O)c1cccc(c1)C(=O)N1CCN(C(=O)CCN3CCCC3=O)CC1CO2] | ND | + | | |
| 305 | 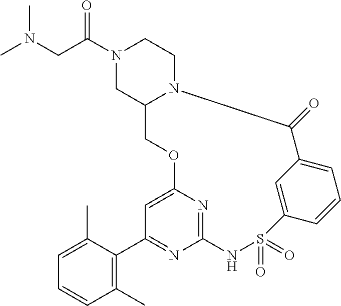 [SMILES: Cc1cccc(C)c1-c1cc2nc(n1)NS(=O)(=O)c1cccc(c1)C(=O)N1CCN(C(=O)CN(C)C)CC1CO2] | ND | + | | |
| 304 | 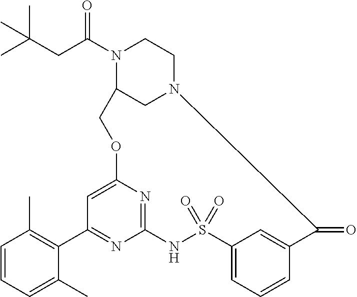 [SMILES: Cc1cccc(C)c1-c1cc2nc(n1)NS(=O)(=O)c1cccc(c1)C(=O)N1CCN(C(=O)CC(C)(C)C)C(CO2)C1] | ND | ++ | | |
| 303 | 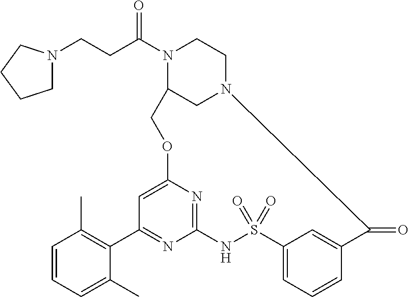 [SMILES: Cc1cccc(C)c1-c1cc2nc(n1)NS(=O)(=O)c1cccc(c1)C(=O)N1CCN(C(=O)CCN3CCCC3)C(CO2)C1] | ND | + | | |
| 302 | 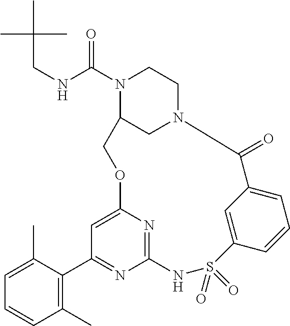 [SMILES: Cc1cccc(C)c1-c1cc2nc(n1)NS(=O)(=O)c1cccc(c1)C(=O)N1CCN(C(=O)NCC(C)(C)C)C(CO2)C1] | ND | + | | |
| 369 | 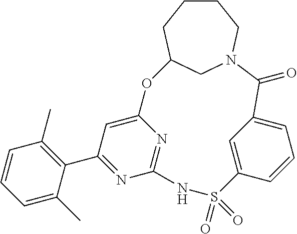 [SMILES: Cc1cccc(C)c1-c1cc2nc(n1)NS(=O)(=O)c1cccc(c1)C(=O)N1CCCCC(C1)O2] | ND | + | | |
| 214 | 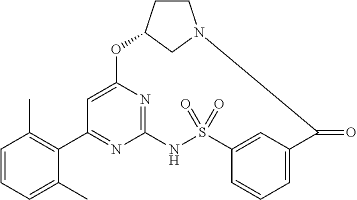 [SMILES: Cc1cccc(C)c1-c1cc2nc(n1)NS(=O)(=O)c1cccc(c1)C(=O)N1CC[C@H](C1)O2] | ND | + | | |
| 213 | 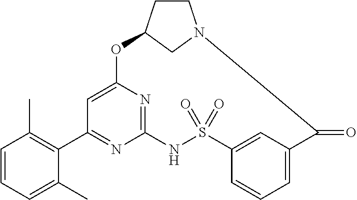 [SMILES: Cc1cccc(C)c1-c1cc2nc(n1)NS(=O)(=O)c1cccc(c1)C(=O)N1CC[C@@H](C1)O2] | + | +++ | | |
| 339 | 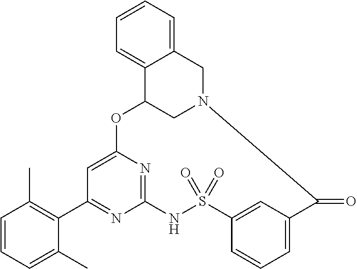 [SMILES: Cc1cccc(C)c1-c1cc2nc(n1)NS(=O)(=O)c1cccc(c1)C(=O)N1Cc3ccccc3C(C1)O2] | +++ | ++ | | |
| 338 | 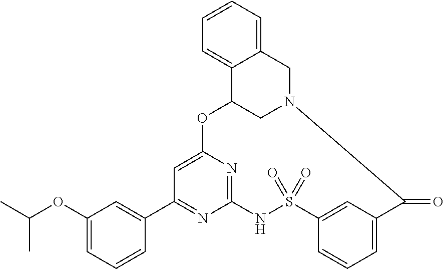 [SMILES: CC(C)Oc1cccc(-c2cc3nc(n2)NS(=O)(=O)c2cccc(c2)C(=O)N2Cc4ccccc4C(C2)O3)c1] | ND | + | | |
| 337 | 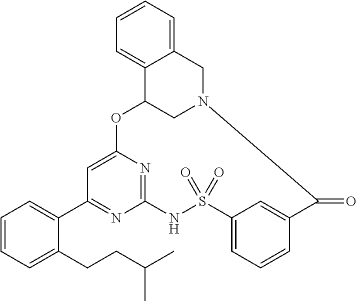 [SMILES: CC(C)CCc1ccccc1-c1cc2nc(n1)NS(=O)(=O)c1cccc(c1)C(=O)N1Cc3ccccc3C(C1)O2] | +++ | ++ | | |
| 367 | 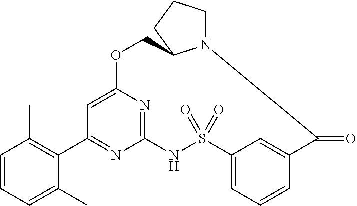 [SMILES: Cc1cccc(C)c1-c1cc2nc(n1)NS(=O)(=O)c1cccc(c1)C(=O)N1CCC[C@@H]1CO2] | + | +++ | | |
| 301 | 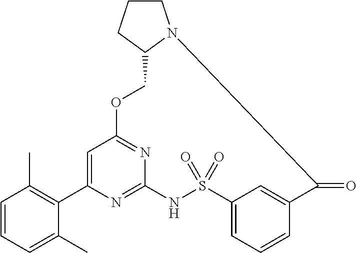 | ND | + | | |
| 300 | 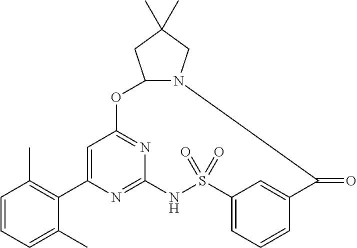 [SMILES: Cc1cccc(C)c1-c1cc2nc(n1)NS(=O)(=O)c1cccc(c1)C(=O)N1CC(C)(C)CC1O2] | + | ++ | | |
| 368 | 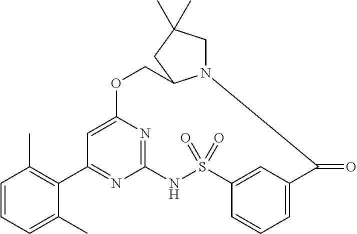 [SMILES: Cc1cccc(C)c1-c1cc2nc(n1)NS(=O)(=O)c1cccc(c1)C(=O)N1CC(C)(C)CC1CO2] | ++ | ++ | | |
| 336 | 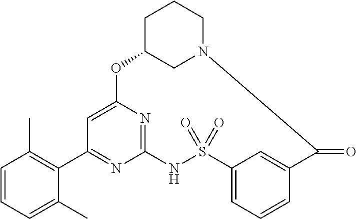 [SMILES: Cc1cccc(C)c1-c1cc2nc(n1)NS(=O)(=O)c1cccc(c1)C(=O)N1CCC[C@H](C1)O2] | ND | + | | |
| 335 | 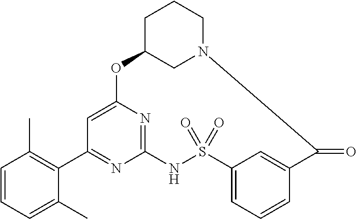 [SMILES: Cc1cccc(C)c1-c1cc2nc(n1)NS(=O)(=O)c1cccc(c1)C(=O)N1CCC[C@@H](C1)O2] | ND | + | | |
| 366 | 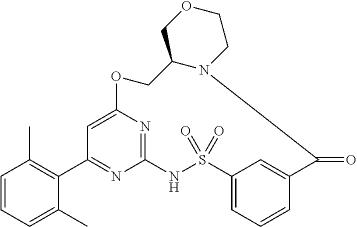 [SMILES: Cc1cccc(C)c1-c1cc2nc(n1)NS(=O)(=O)c1cccc(c1)C(=O)N1CCOC[C@@H]1CO2] | + | ++ | | |
| 229 | 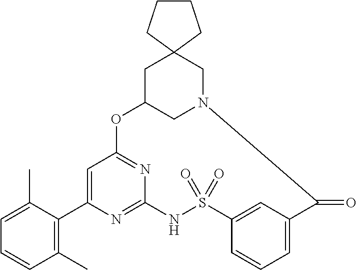 [SMILES: Cc1cccc(C)c1-c1cc2nc(n1)NS(=O)(=O)c1cccc(c1)C(=O)N1CC(CC3(CCCC3)C1)O2] | +++ | ++ | | |
| 43 | 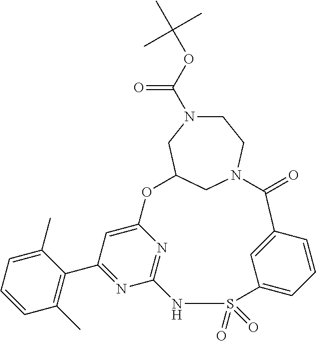 [SMILES: Cc1cccc(C)c1-c1cc2nc(n1)NS(=O)(=O)c1cccc(c1)C(=O)N1CCN(C(=O)OC(C)(C)C)CC(C1)O2] | +++ | +++ | | |
| 42 | 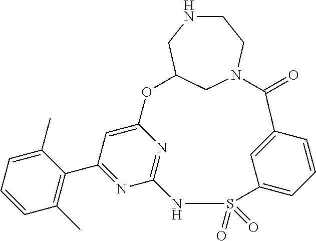 [SMILES: Cc1cccc(C)c1-c1cc2nc(n1)NS(=O)(=O)c1cccc(c1)C(=O)N1CCNCC(C1)O2] | ND | ++ | | |
| 220 | 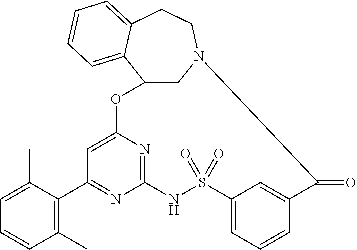 [SMILES: Cc1cccc(C)c1-c1cc2nc(n1)NS(=O)(=O)c1cccc(c1)C(=O)N1CCc3ccccc3C(C1)O2] | +++ | +++ | | |
| 161 | 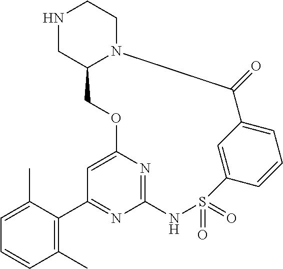 [SMILES: Cc1cccc(C)c1-c1cc2nc(n1)NS(=O)(=O)c1cccc(c1)C(=O)N1CCNC[C@@H]1CO2] | + | ++ | | |
| 299 | 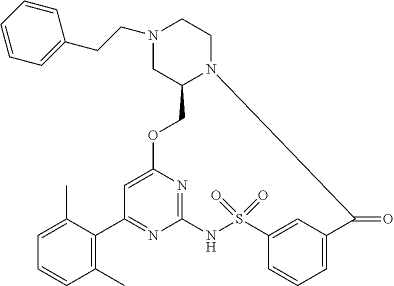 [SMILES: Cc1cccc(C)c1-c1cc2nc(n1)NS(=O)(=O)c1cccc(c1)C(=O)N1CCN(CCc3ccccc3)C[C@@H]1CO2] | +++ | ++ | | |
| 298 | 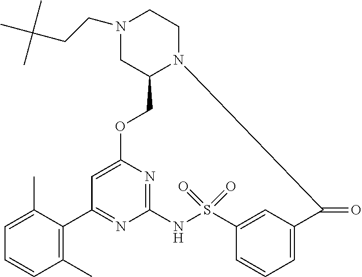 [SMILES: Cc1cccc(C)c1-c1cc2nc(n1)NS(=O)(=O)c1cccc(c1)C(=O)N1CCN(CCC(C)(C)C)C[C@@H]1CO2] | +++ | +++ | +++ | +++ |
| 297 | 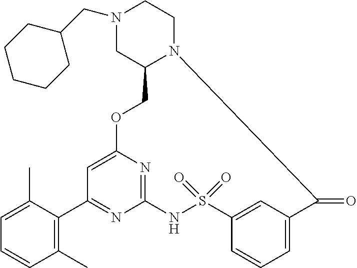 [SMILES: Cc1cccc(C)c1-c1cc2nc(n1)NS(=O)(=O)c1cccc(c1)C(=O)N1CCN(CC3CCCCC3)C[C@@H]1CO2] | +++ | +++ | | |
| 164 | 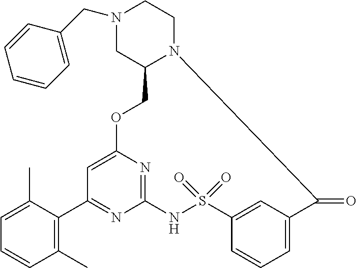 [SMILES: Cc1cccc(C)c1-c1cc2nc(n1)NS(=O)(=O)c1cccc(c1)C(=O)N1CCN(Cc3ccccc3)C[C@@H]1CO2] | +++ | +++ | | |
| 165 | 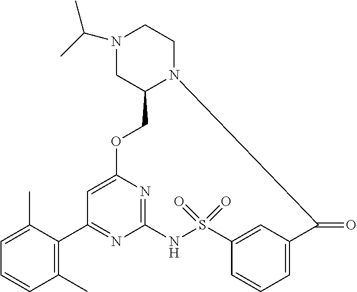 [SMILES: Cc1cccc(C)c1-c1cc2nc(n1)NS(=O)(=O)c1cccc(c1)C(=O)N1CCN(C(C)C)C[C@@H]1CO2] | + | +++ | | |
| 163 | 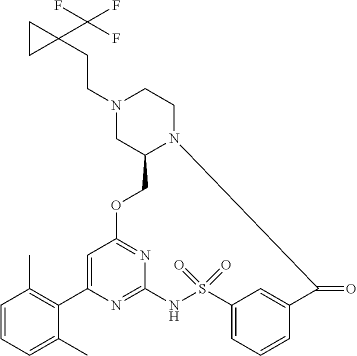 [SMILES: Cc1cccc(C)c1-c1cc2nc(n1)NS(=O)(=O)c1cccc(c1)C(=O)N1CCN(CCC3(C(F)(F)F)CC3)C[C@@H]1CO2] | +++ | +++ | | |
| 296 | 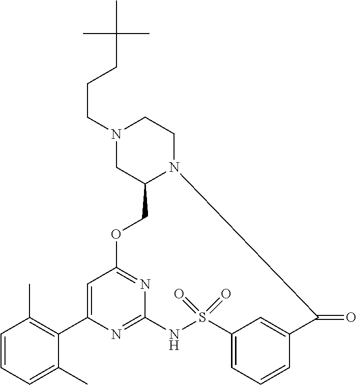 [SMILES: Cc1cccc(C)c1-c1cc2nc(n1)NS(=O)(=O)c1cccc(c1)C(=O)N1CCN(CCCC(C)(C)C)C[C@@H]1CO2] | +++ | +++ | | |
| 295 | 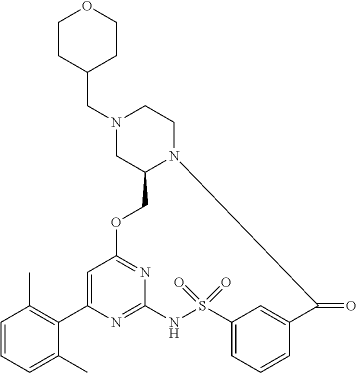 [SMILES: Cc1cccc(C)c1-c1cc2nc(n1)NS(=O)(=O)c1cccc(c1)C(=O)N1CCN(CC3CCOCC3)C[C@@H]1CO2] | ++ | +++ | | |
| 294 | 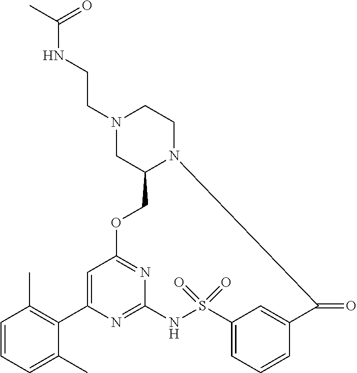 [SMILES: CC(=O)NCCN1CCN2C(=O)c3cccc(c3)S(=O)(=O)Nc3nc(cc(-c4c(C)cccc4C)n3)OC[C@H]2C1] | + | ++ | | |
| 293 | 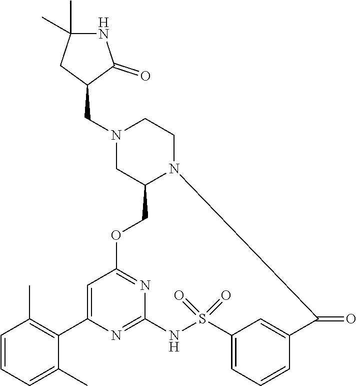 [SMILES: Cc1cccc(C)c1-c1cc2nc(n1)NS(=O)(=O)c1cccc(c1)C(=O)N1CCN(C[C@H]3CC(C)(C)NC3=O)C[C@@H]1CO2] | ND | + | | |
| 63 | 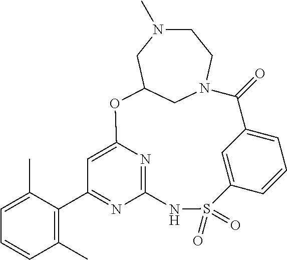 [SMILES: Cc1cccc(C)c1-c1cc2nc(n1)NS(=O)(=O)c1cccc(c1)C(=O)N1CCN(C)CC(C1)O2] | + | +++ | | |
| 44 | 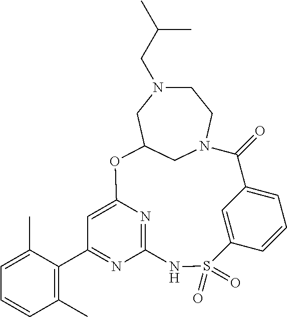 [SMILES: Cc1cccc(C)c1-c1cc2nc(n1)NS(=O)(=O)c1cccc(c1)C(=O)N1CCN(CC(C)C)CC(C1)O2] | +++ | +++ | | |
| 62 | 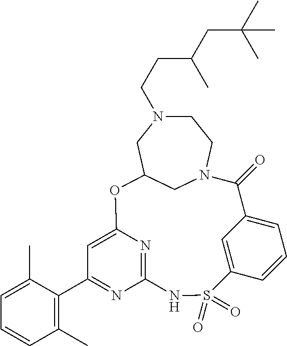 [SMILES: Cc1cccc(C)c1-c1cc2nc(n1)NS(=O)(=O)c1cccc(c1)C(=O)N1CCN(CCC(C)CC(C)(C)C)CC(C1)O2] | +++ | +++ | | |
| 61 | 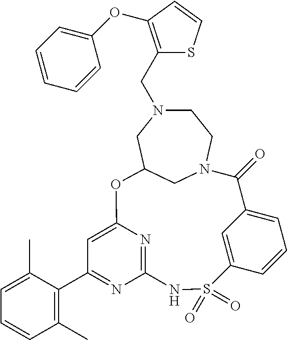 [SMILES: Cc1cccc(C)c1-c1cc2nc(n1)NS(=O)(=O)c1cccc(c1)C(=O)N1CCN(Cc3sccc3Oc3ccccc3)CC(C1)O2] | +++ | +++ | | |
| 60 | 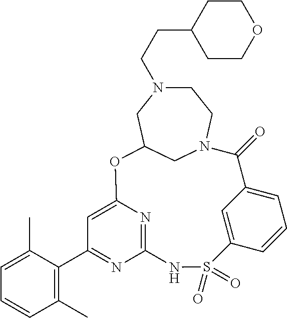 [SMILES: Cc1cccc(C)c1-c1cc2nc(n1)NS(=O)(=O)c1cccc(c1)C(=O)N1CCN(CCC3CCOCC3)CC(C1)O2] | + | +++ | | |
| 58 | 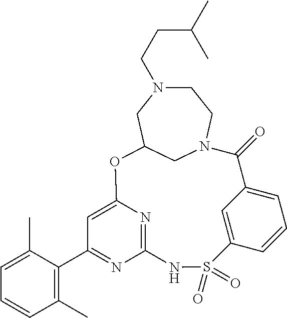 [SMILES: Cc1cccc(C)c1-c1cc2nc(n1)NS(=O)(=O)c1cccc(c1)C(=O)N1CCN(CCC(C)C)CC(C1)O2] | +++ | +++ | | |
| 215 | 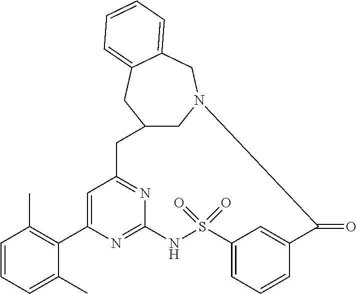 [SMILES: Cc1cccc(C)c1-c1cc2nc(n1)NS(=O)(=O)c1cccc(c1)C(=O)N1Cc3ccccc3CC(C2)C1] | +++ | ++ | | |
| 166 | 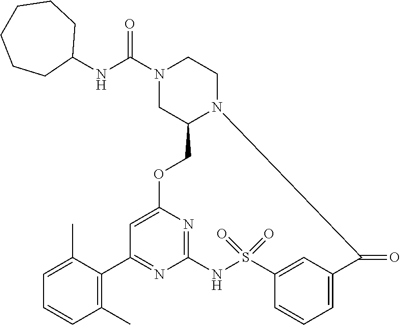 [SMILES: Cc1cccc(C)c1-c1cc2nc(n1)NS(=O)(=O)c1cccc(c1)C(=O)N1CCN(C(=O)NC3CCCCCC3)C[C@@H]1CO2] | + | +++ | | |
| 292 | 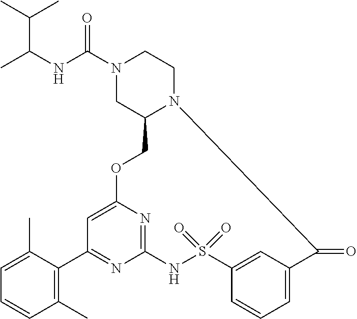 [SMILES: Cc1cccc(C)c1-c1cc2nc(n1)NS(=O)(=O)c1cccc(c1)C(=O)N1CCN(C(=O)NC(C)C(C)C)C[C@@H]1CO2] | ND | ++ | | |
| 291 | 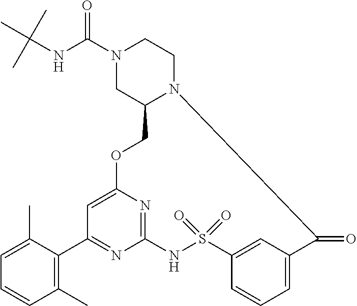 [SMILES: Cc1cccc(C)c1-c1cc2nc(n1)NS(=O)(=O)c1cccc(c1)C(=O)N1CCN(C(=O)NC(C)(C)C)C[C@@H]1CO2] | ND | +++ | | |
| 290 | 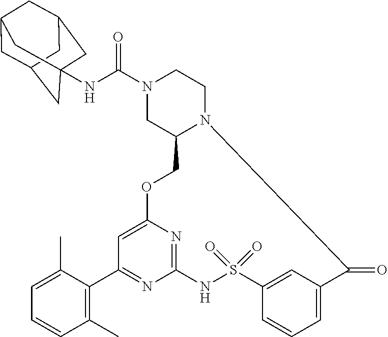 [SMILES: Cc1cccc(C)c1-c1cc2nc(n1)NS(=O)(=O)c1cccc(c1)C(=O)N1CCN(C(=O)NC34CC5CC(CC(C5)C3)C4)C[C@@H]1CO2] | ++ | ++ | | |
| 289 | 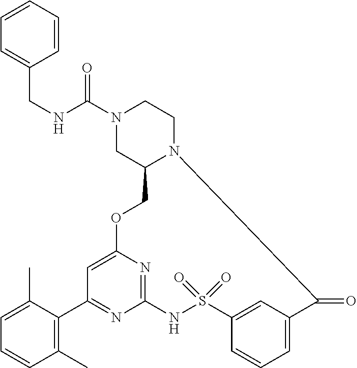 [SMILES: Cc1cccc(C)c1-c1cc2nc(n1)NS(=O)(=O)c1cccc(c1)C(=O)N1CCN(C(=O)NCc3ccccc3)C[C@@H]1CO2] | ND | + | | |
| 167 | 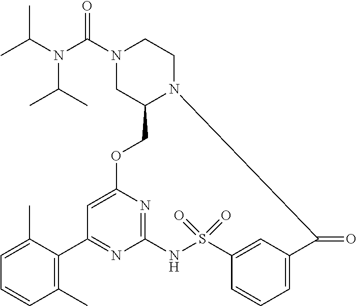 [SMILES: Cc1cccc(C)c1-c1cc2nc(n1)NS(=O)(=O)c1cccc(c1)C(=O)N1CCN(C(=O)N(C(C)C)C(C)C)C[C@@H]1CO2] | ++ | +++ | | |
| 258 | 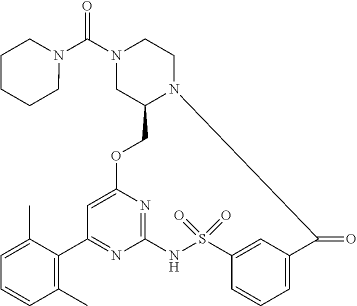 [SMILES: Cc1cccc(C)c1-c1cc2nc(n1)NS(=O)(=O)c1cccc(c1)C(=O)N1CCN(C(=O)N3CCCCC3)C[C@@H]1CO2] | + | ++ | | |
| 59 | 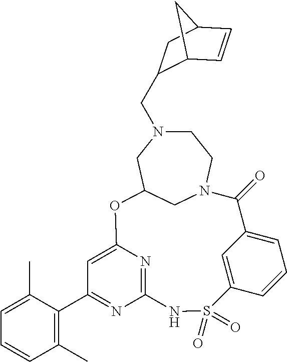 [SMILES: Cc1cccc(C)c1-c1cc2nc(n1)NS(=O)(=O)c1cccc(c1)C(=O)N1CCN(CC3CC4C=CC3C4)CC(C1)O2] | +++ | +++ | | |
| 55 | 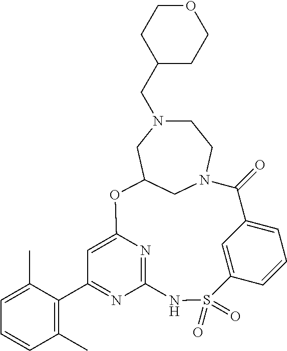 [SMILES: Cc1cccc(C)c1-c1cc2nc(n1)NS(=O)(=O)c1cccc(c1)C(=O)N1CCN(CC3CCOCC3)CC(C1)O2] | + | +++ | | |
| 54 | 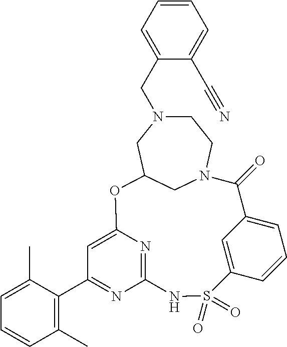 [SMILES: Cc1cccc(C)c1-c1cc2nc(n1)NS(=O)(=O)c1cccc(c1)C(=O)N1CCN(Cc3ccccc3C#N)CC(C1)O2] | +++ | +++ | | |
| 51 | 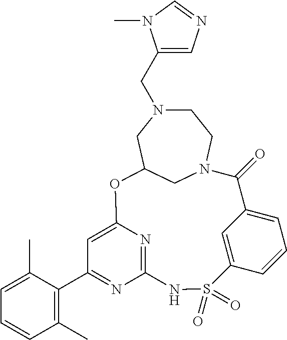 [SMILES: Cc1cccc(C)c1-c1cc2nc(n1)NS(=O)(=O)c1cccc(c1)C(=O)N1CCN(Cc3cncn3C)CC(C1)O2] | ND | + | | |
| 50 | 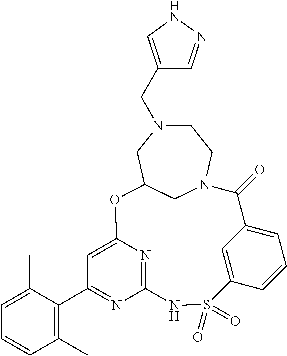 [SMILES: Cc1cccc(C)c1-c1cc2nc(n1)NS(=O)(=O)c1cccc(c1)C(=O)N1CCN(Cc3cn[nH]c3)CC(C1)O2] | ND | + | | |
| 288 | 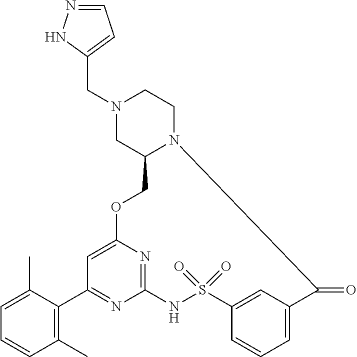 [SMILES: Cc1cccc(C)c1-c1cc2nc(n1)NS(=O)(=O)c1cccc(c1)C(=O)N1CCN(Cc3ccn[nH]3)C[C@@H]1CO2] | ND | + | | |
| 284 | 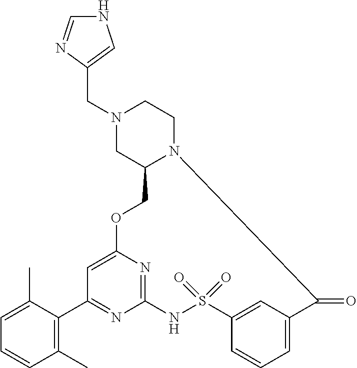 [SMILES: Cc1cccc(C)c1-c1cc2nc(n1)NS(=O)(=O)c1cccc(c1)C(=O)N1CCN(Cc3c[nH]cn3)C[C@@H]1CO2] | ND | + | | |
| 283 | 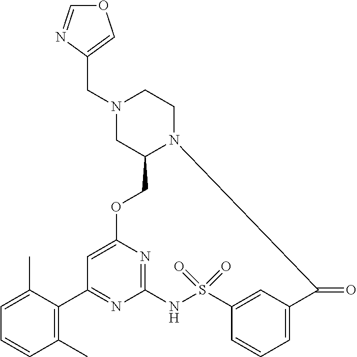 [SMILES: Cc1cccc(C)c1-c1cc2nc(n1)NS(=O)(=O)c1cccc(c1)C(=O)N1CCN(Cc3cocn3)C[C@@H]1CO2] | ND | ++ | | |
| 282 | 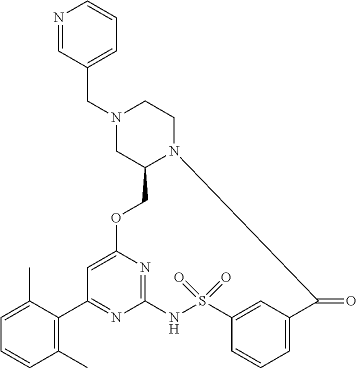 [SMILES: Cc1cccc(C)c1-c1cc2nc(n1)NS(=O)(=O)c1cccc(c1)C(=O)N1CCN(Cc3cccnc3)C[C@@H]1CO2] | + | ++ | | |
| 281 | 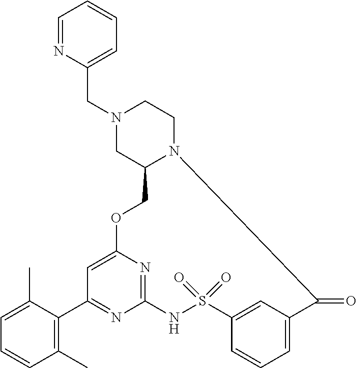 [SMILES: Cc1cccc(C)c1-c1cc2nc(n1)NS(=O)(=O)c1cccc(c1)C(=O)N1CCN(Cc3ccccn3)C[C@@H]1CO2] | ND | ++ | | |
| 280 | 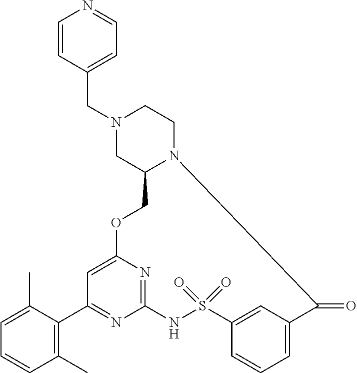 [SMILES: Cc1cccc(C)c1-c1cc2nc(n1)NS(=O)(=O)c1cccc(c1)C(=O)N1CCN(Cc3ccncc3)C[C@@H]1CO2] | ND | ++ | | |
| 287 | 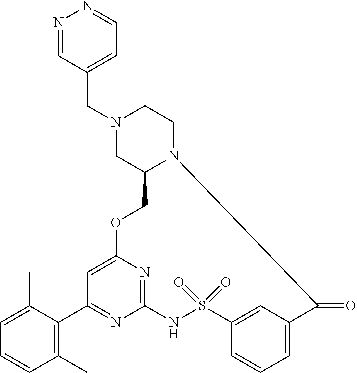 [SMILES: Cc1cccc(C)c1-c1cc2nc(n1)NS(=O)(=O)c1cccc(c1)C(=O)N1CCN(Cc3ccnnc3)C[C@@H]1CO2] | ND | + | | |
| 286 | 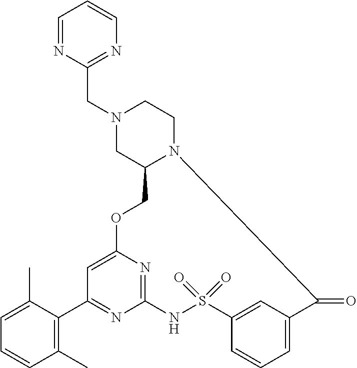 [SMILES: Cc1cccc(C)c1-c1cc2nc(n1)NS(=O)(=O)c1cccc(c1)C(=O)N1CCN(Cc3ncccn3)C[C@@H]1CO2] | ND | + | | |
| 285 | 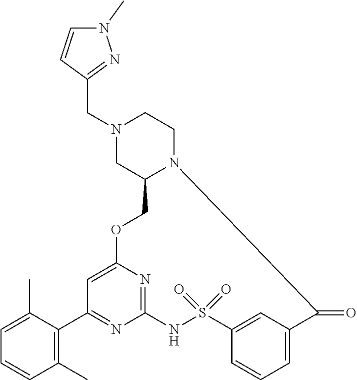 [SMILES: Cc1cccc(C)c1-c1cc2nc(n1)NS(=O)(=O)c1cccc(c1)C(=O)N1CCN(Cc3ccn(C)n3)C[C@@H]1CO2] | ND | ++ | | |
| 279 | 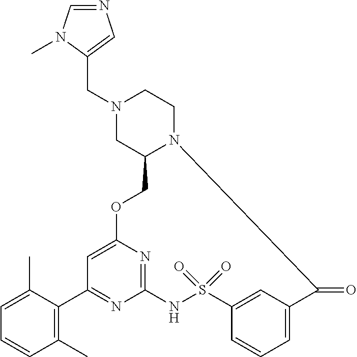 [SMILES: Cc1cccc(C)c1-c1cc2nc(n1)NS(=O)(=O)c1cccc(c1)C(=O)N1CCN(Cc3cncn3C)C[C@@H]1CO2] | ND | + | | |
| 275 | 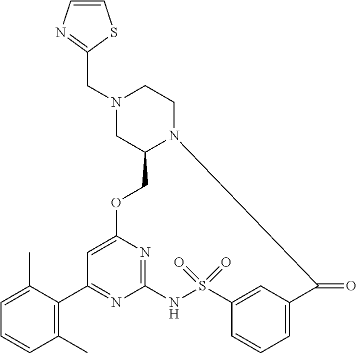 [SMILES: Cc1cccc(C)c1-c1cc2nc(n1)NS(=O)(=O)c1cccc(c1)C(=O)N1CCN(Cc3nccs3)C[C@@H]1CO2] | + | +++ | | |
| 274 | 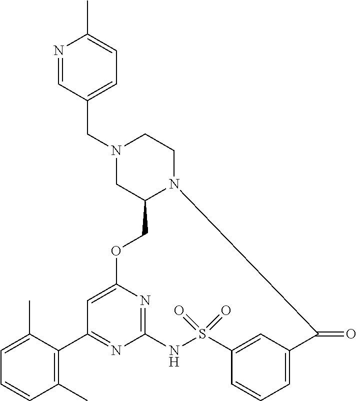 [SMILES: Cc1ccc(CN2CCN3C(=O)c4cccc(c4)S(=O)(=O)Nc4nc(cc(-c5c(C)cccc5C)n4)OC[C@H]3C2)cn1] | + | +++ | | |
| 273 | 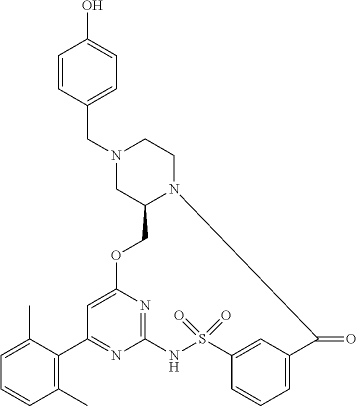 [SMILES: Cc1cccc(C)c1-c1cc2nc(n1)NS(=O)(=O)c1cccc(c1)C(=O)N1CCN(Cc3ccc(O)cc3)C[C@@H]1CO2] | ++ | ++ | | |
| 272 | 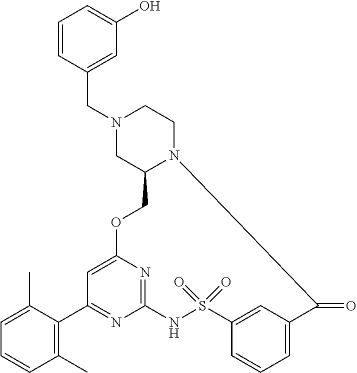 [SMILES: Cc1cccc(C)c1-c1cc2nc(n1)NS(=O)(=O)c1cccc(c1)C(=O)N1CCN(Cc3cccc(O)c3)C[C@@H]1CO2] | + | +++ | | |
| 271 | 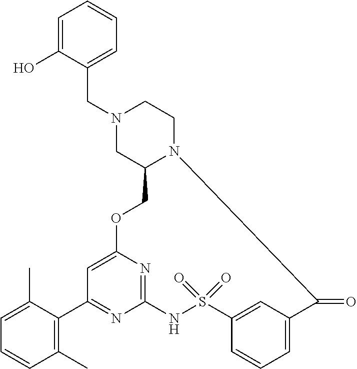 [SMILES: Cc1cccc(C)c1-c1cc2nc(n1)NS(=O)(=O)c1cccc(c1)C(=O)N1CCN(Cc3ccccc3O)C[C@@H]1CO2] | +++ | +++ | | |
| 278 | 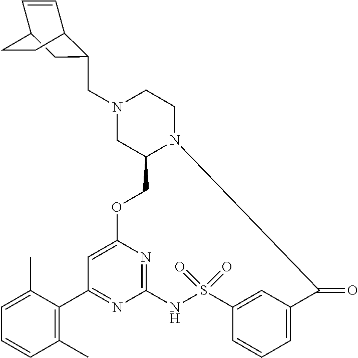 [SMILES: Cc1cccc(C)c1-c1cc2nc(n1)NS(=O)(=O)c1cccc(c1)C(=O)N1CCN(CC3CC4C=CC3CC4)C[C@@H]1CO2] | +++ | ++ | | |
| 277 | 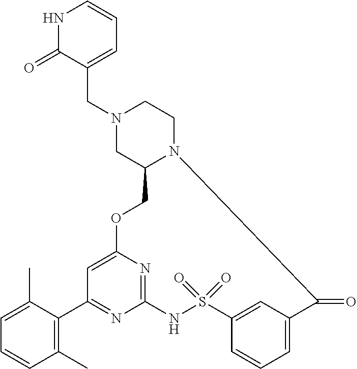 [SMILES: Cc1cccc(C)c1-c1cc2nc(n1)NS(=O)(=O)c1cccc(c1)C(=O)N1CCN(Cc3ccc[nH]c3=O)C[C@@H]1CO2] | ND | + | | |
| 276 | 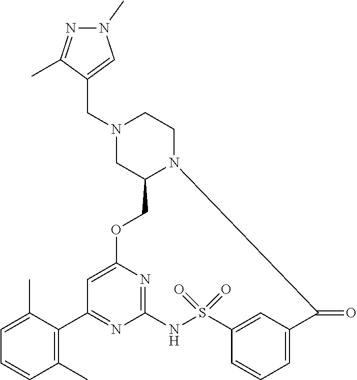 [SMILES: Cc1cccc(C)c1-c1cc2nc(n1)NS(=O)(=O)c1cccc(c1)C(=O)N1CCN(Cc3cn(C)nc3C)C[C@@H]1CO2] | ND | ++ | | |
| 270 | 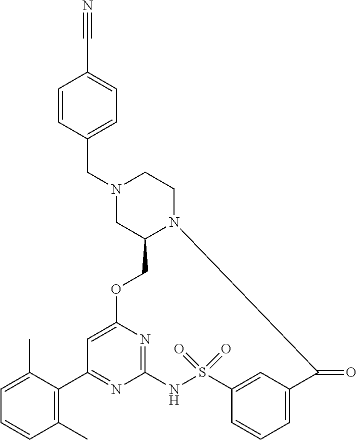 [SMILES: Cc1cccc(C)c1-c1cc2nc(n1)NS(=O)(=O)c1cccc(c1)C(=O)N1CCN(Cc3ccc(C#N)cc3)C[C@@H]1CO2] | ++ | +++ | | |
| 267 | 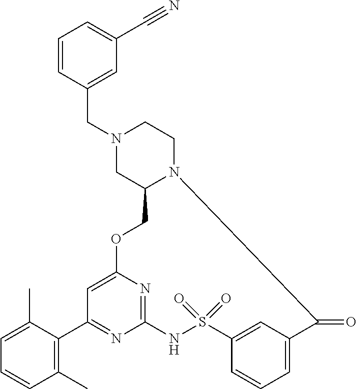 [SMILES: Cc1cccc(C)c1-c1cc2nc(n1)NS(=O)(=O)c1cccc(c1)C(=O)N1CCN(Cc3cccc(C#N)c3)C[C@@H]1CO2] | ++ | +++ | | |
| 266 | 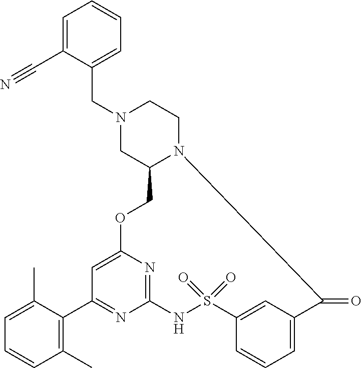 [SMILES: Cc1cccc(C)c1-c1cc2nc(n1)NS(=O)(=O)c1cccc(c1)C(=O)N1CCN(Cc3ccccc3C#N)C[C@@H]1CO2] | + | +++ | | |
| 265 | 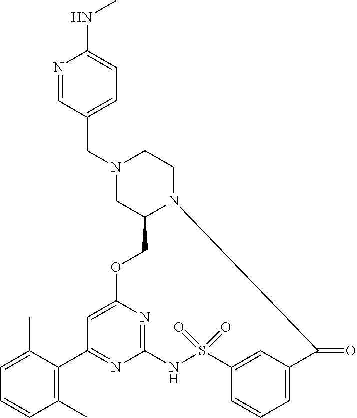 [SMILES: CNc1ccc(CN2CCN3C(=O)c4cccc(c4)S(=O)(=O)Nc4nc(cc(-c5c(C)cccc5C)n4)OC[C@H]3C2)cn1] | + | ++ | | |
| 264 | 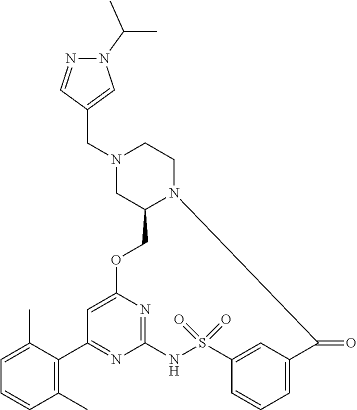 [SMILES: Cc1cccc(C)c1-c1cc2nc(n1)NS(=O)(=O)c1cccc(c1)C(=O)N1CCN(Cc3cnn(C(C)C)c3)C[C@@H]1CO2] | ND | ++ | | |
| 263 | 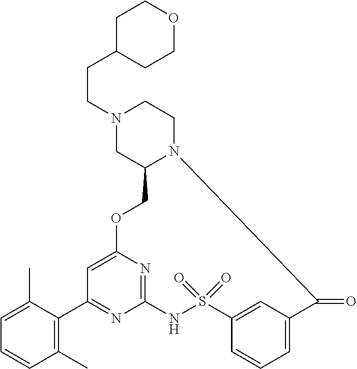 [SMILES: Cc1cccc(C)c1-c1cc2nc(n1)NS(=O)(=O)c1cccc(c1)C(=O)N1CCN(CCC3CCOCC3)C[C@@H]1CO2] | + | ++ | | |
| 262 | 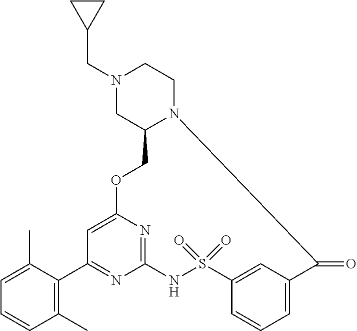 [SMILES: Cc1cccc(C)c1-c1cc2nc(n1)NS(=O)(=O)c1cccc(c1)C(=O)N1CCN(CC3CC3)C[C@@H]1CO2] | + | +++ | | |
| 269 | 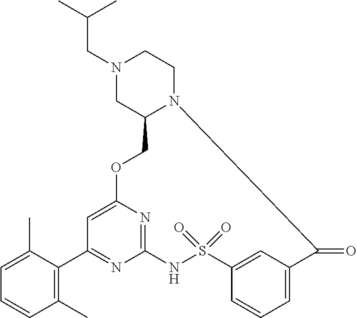 [SMILES: Cc1cccc(C)c1-c1cc2nc(n1)NS(=O)(=O)c1cccc(c1)C(=O)N1CCN(CC(C)C)C[C@@H]1CO2] | +++ | +++ | | |
| 268 | 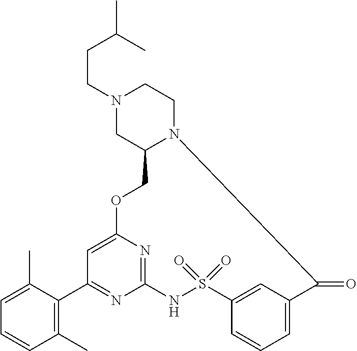 [SMILES: Cc1cccc(C)c1-c1cc2nc(n1)NS(=O)(=O)c1cccc(c1)C(=O)N1CCN(CCC(C)C)C[C@@H]1CO2] | ++ | +++ | | |
| 261 | 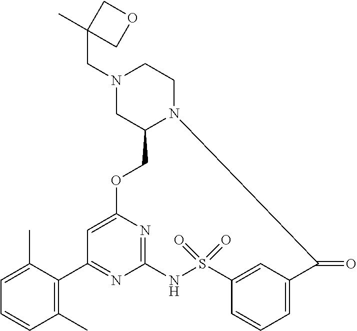 [SMILES: Cc1cccc(C)c1-c1cc2nc(n1)NS(=O)(=O)c1cccc(c1)C(=O)N1CCN(CC3(C)COC3)C[C@@H]1CO2] | ND | + | | |
| 260 | 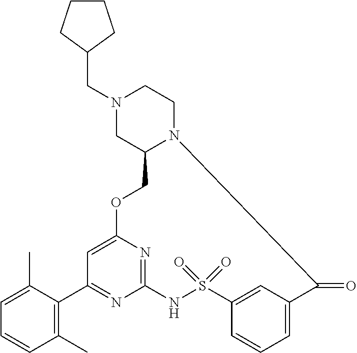 [SMILES: Cc1cccc(C)c1-c1cc2nc(n1)NS(=O)(=O)c1cccc(c1)C(=O)N1CCN(CC3CCCC3)C[C@@H]1CO2] | +++ | +++ | | |
| 259 | 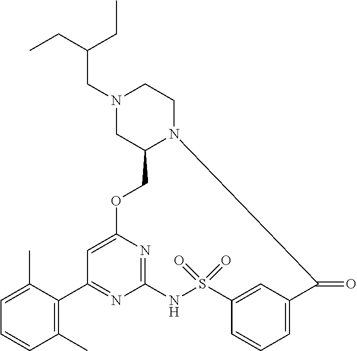 [SMILES: CCC(CC)CN1CCN2C(=O)c3cccc(c3)S(=O)(=O)Nc3nc(cc(-c4c(C)cccc4C)n3)OC[C@H]2C1] | +++ | +++ | | |
| 365 | 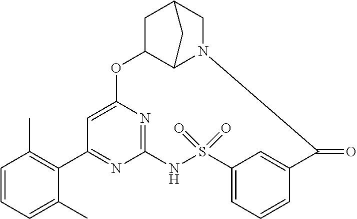 [SMILES: Cc1cccc(C)c1-c1cc2nc(n1)NS(=O)(=O)c1cccc(c1)C(=O)N1CC3CC(O2)C1C3] | + | ++ | | |
| 47 | 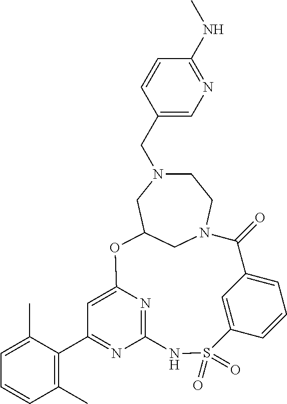 [SMILES: CNc1ccc(CN2CCN3CC(C2)Oc2cc(-c4c(C)cccc4C)nc(n2)NS(=O)(=O)c2cccc(c2)C3=O)cn1] | + | +++ | | |
| 53 | 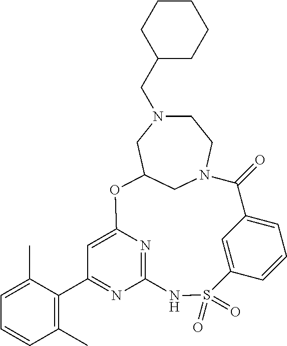 [SMILES: Cc1cccc(C)c1-c1cc2nc(n1)NS(=O)(=O)c1cccc(c1)C(=O)N1CCN(CC3CCCCC3)CC(C1)O2] | +++ | ++ | | |
| 57 | 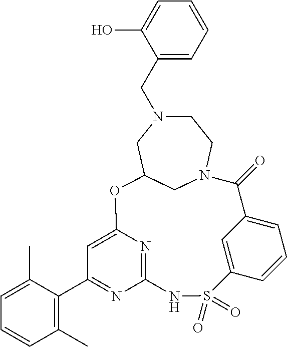 [SMILES: Cc1cccc(C)c1-c1cc2nc(n1)NS(=O)(=O)c1cccc(c1)C(=O)N1CCN(Cc3ccccc3O)CC(C1)O2] | +++ | +++ | | |
| 56 | 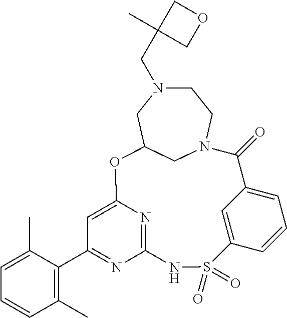 [SMILES: Cc1cccc(C)c1-c1cc2nc(n1)NS(=O)(=O)c1cccc(c1)C(=O)N1CCN(CC3(C)COC3)CC(C1)O2] | + | ++ | | |
| 66 | 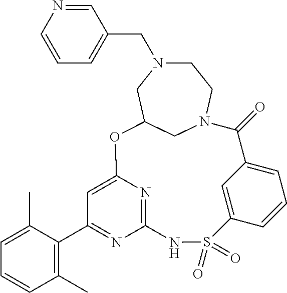 [SMILES: Cc1cccc(C)c1-c1cc2nc(n1)NS(=O)(=O)c1cccc(c1)C(=O)N1CCN(Cc3cccnc3)CC(C1)O2] | + | ++ | | |
| 162 | 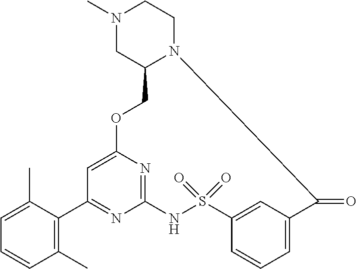 [SMILES: Cc1cccc(C)c1-c1cc2nc(n1)NS(=O)(=O)c1cccc(c1)C(=O)N1CCN(C)C[C@@H]1CO2] | + | ++ | | |
| 256 | 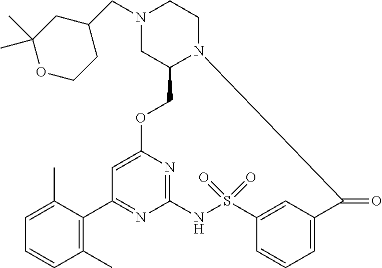 [SMILES: Cc1cccc(C)c1-c1cc2nc(n1)NS(=O)(=O)c1cccc(c1)C(=O)N1CCN(CC3CCOC(C)(C)C3)C[C@@H]1CO2] | +++ | +++ | | |
| 168 | 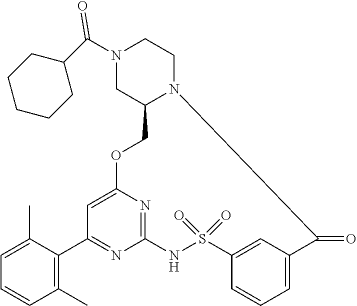 [SMILES: Cc1cccc(C)c1-c1cc2nc(n1)NS(=O)(=O)c1cccc(c1)C(=O)N1CCN(C(=O)C3CCCCC3)C[C@@H]1CO2] | +++ | +++ | | |
| 255 | 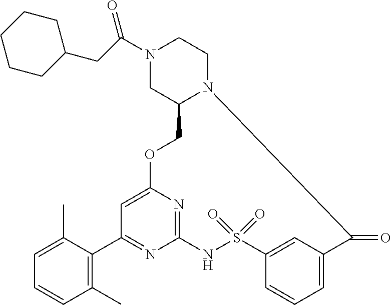 [SMILES: Cc1cccc(C)c1-c1cc2nc(n1)NS(=O)(=O)c1cccc(c1)C(=O)N1CCN(C(=O)CC3CCCCC3)C[C@@H]1CO2] | +++ | ++ | | |
| 257 | 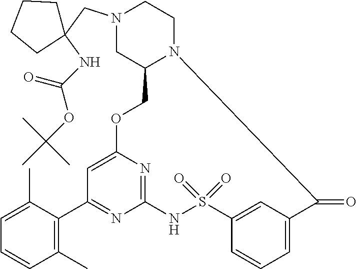 [SMILES: Cc1cccc(C)c1-c1cc2nc(n1)NS(=O)(=O)c1cccc(c1)C(=O)N1CCN(CC3(NC(=O)OC(C)(C)C)CCCC3)C[C@@H]1CO2] | +++ | +++ | | |
| 363 | 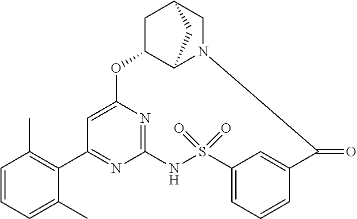 [SMILES: Cc1cccc(C)c1-c1cc2nc(n1)NS(=O)(=O)c1cccc(c1)C(=O)N1C[C@H]3C[C@@H]1[C@@H](C3)O2] | + | +++ | | |
| 364 | 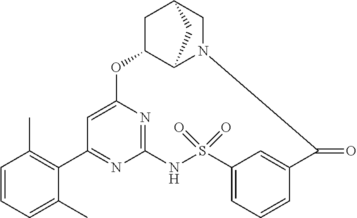 [SMILES: Cc1cccc(C)c1-c1cc2nc(n1)NS(=O)(=O)c1cccc(c1)C(=O)N1C[C@H]3C[C@@H]1[C@@H](C3)O2] | ND | ++ | | |
| 52 | 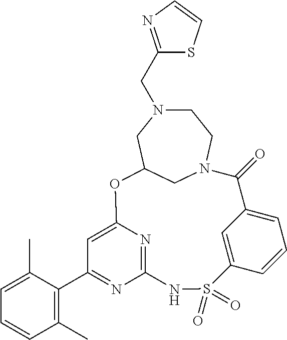 [SMILES: Cc1cccc(C)c1-c1cc2nc(n1)NS(=O)(=O)c1cccc(c1)C(=O)N1CCN(Cc3nccs3)CC(C1)O2] | ++ | ++ | | |
| 64 | 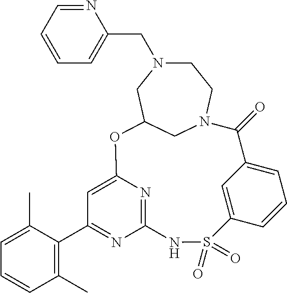 [SMILES: Cc1cccc(C)c1-c1cc2nc(n1)NS(=O)(=O)c1cccc(c1)C(=O)N1CCN(Cc3ccccn3)CC(C1)O2] | ++ | ++ | | |
| 65 | 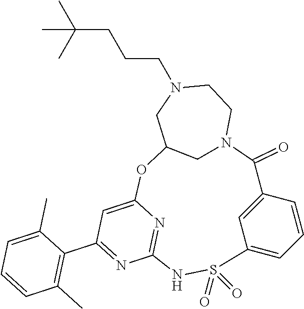 [SMILES: Cc1cccc(C)c1-c1cc2nc(n1)NS(=O)(=O)c1cccc(c1)C(=O)N1CCN(CCCC(C)(C)C)CC(C1)O2] | +++ | ++ | | |
| 216 | 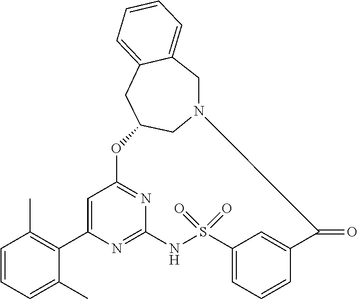 [SMILES: Cc1cccc(C)c1-c1cc2nc(n1)NS(=O)(=O)c1cccc(c1)C(=O)N1Cc3ccccc3C[C@H](C1)O2] | ++ | +++ | | |
| 217 | 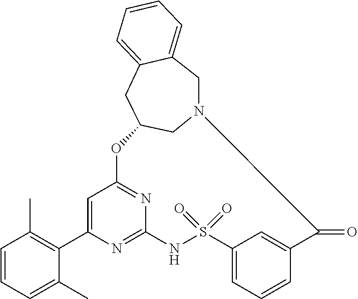 [SMILES: Cc1cccc(C)c1-c1cc2nc(n1)NS(=O)(=O)c1cccc(c1)C(=O)N1Cc3ccccc3C[C@H](C1)O2] | ND | + | | |
| 254 | 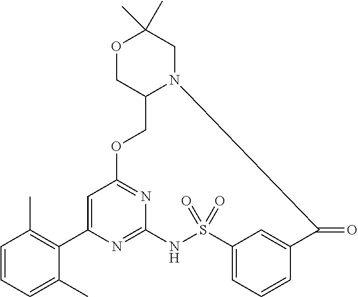 [SMILES: Cc1cccc(C)c1-c1cc2nc(n1)NS(=O)(=O)c1cccc(c1)C(=O)N1CC(C)(C)OCC1CO2] | ++ | ++ | | |
| 67 | 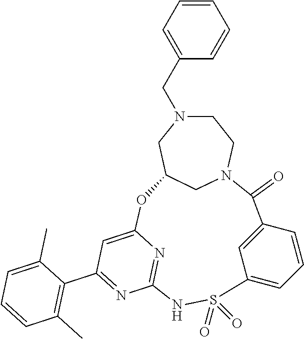 [SMILES: Cc1cccc(C)c1-c1cc2nc(n1)NS(=O)(=O)c1cccc(c1)C(=O)N1CCN(Cc3ccccc3)C[C@H](C1)O2] | +++ | +++ | | |
| 68 | 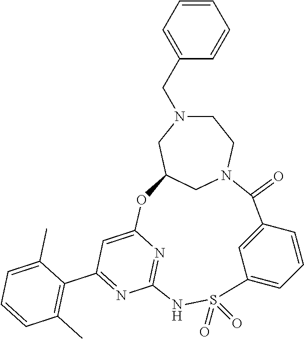 [SMILES: Cc1cccc(C)c1-c1cc2nc(n1)NS(=O)(=O)c1cccc(c1)C(=O)N1CCN(Cc3ccccc3)C[C@@H](C1)O2] | ++ | + | | |
| 45 | 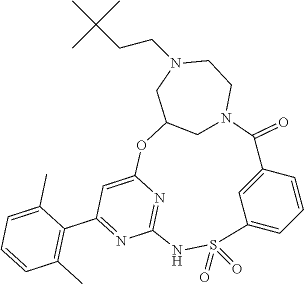 [SMILES: Cc1cccc(C)c1-c1cc2nc(n1)NS(=O)(=O)c1cccc(c1)C(=O)N1CCN(CCC(C)(C)C)CC(C1)O2] | +++ | +++ | | |
| 49 | 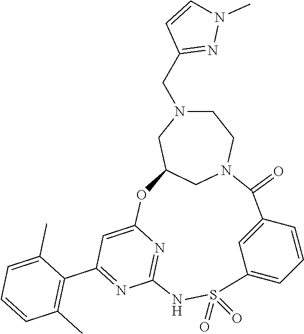 [SMILES: Cc1cccc(C)c1-c1cc2nc(n1)NS(=O)(=O)c1cccc(c1)C(=O)N1CCN(Cc3ccn(C)n3)C[C@@H](C1)O2] | + | +++ | | |
| 48 | 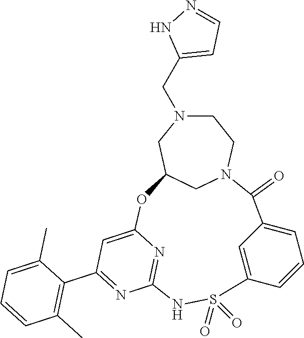 [SMILES: Cc1cccc(C)c1-c1cc2nc(n1)NS(=O)(=O)c1cccc(c1)C(=O)N1CCN(Cc3ccn[nH]3)C[C@@H](C1)O2] | + | ++ | | |
| 226 | 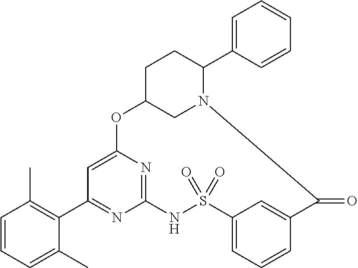 [SMILES: Cc1cccc(C)c1-c1cc2nc(n1)NS(=O)(=O)c1cccc(c1)C(=O)N1CC(CCC1c1ccccc1)O2] | +++ | ++ | | |
| 223 | 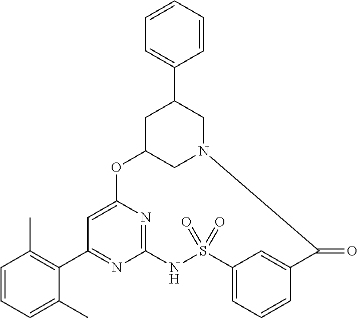 [SMILES: Cc1cccc(C)c1-c1cc2nc(n1)NS(=O)(=O)c1cccc(c1)C(=O)N1CC(CC(c3ccccc3)C1)O2] | +++ | ++ | | |
| 224 | 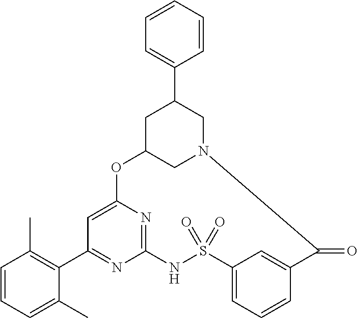 [SMILES: Cc1cccc(C)c1-c1cc2nc(n1)NS(=O)(=O)c1cccc(c1)C(=O)N1CC(CC(c3ccccc3)C1)O2] | +++ | ++ | | |
| 227 | 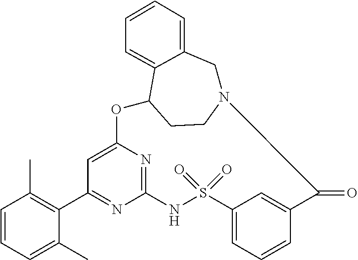 [SMILES: Cc1cccc(C)c1-c1cc2nc(n1)NS(=O)(=O)c1cccc(c1)C(=O)N1CCC(O2)c2ccccc2C1] | +++ | ++ | | |
| 225 | 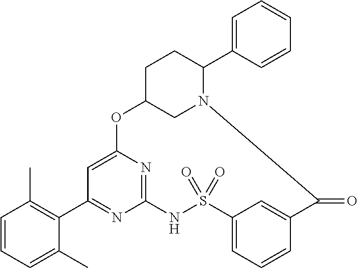 [SMILES: Cc1cccc(C)c1-c1cc2nc(n1)NS(=O)(=O)c1cccc(c1)C(=O)N1CC(CCC1c1ccccc1)O2] | +++ | ++ | | |
| 332 | 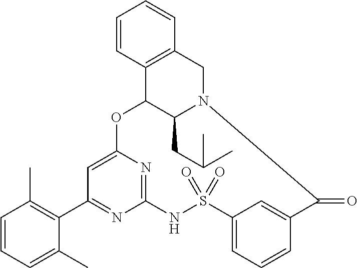 [SMILES: Cc1cccc(C)c1-c1cc2nc(n1)NS(=O)(=O)c1cccc(c1)C(=O)N1Cc3ccccc3C(O2)[C@@H]1CC(C)C] | +++ | +++ | | |
| 333 | 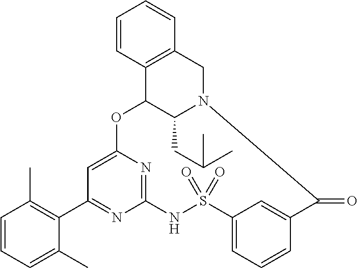 [SMILES: Cc1cccc(C)c1-c1cc2nc(n1)NS(=O)(=O)c1cccc(c1)C(=O)N1Cc3ccccc3C(O2)[C@H]1CC(C)C] | +++ | +++ | | |
| 1 | 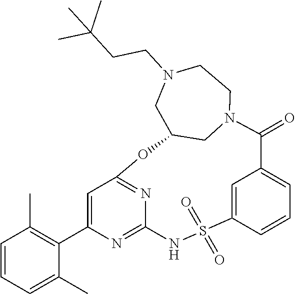 [SMILES: Cc1cccc(C)c1-c1cc2nc(n1)NS(=O)(=O)c1cccc(c1)C(=O)N1CCN(CCC(C)(C)C)C[C@H](C1)O2] | +++ | +++ | +++ | +++ |
| 360 | 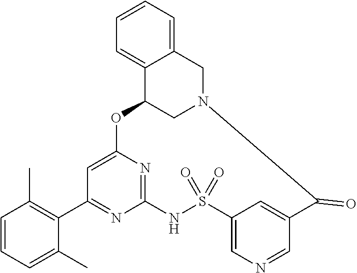 [SMILES: Cc1cccc(C)c1-c1cc2nc(n1)NS(=O)(=O)c1cncc(c1)C(=O)N1Cc3ccccc3[C@@H](C1)O2] | ND | + | | |
| 361 | 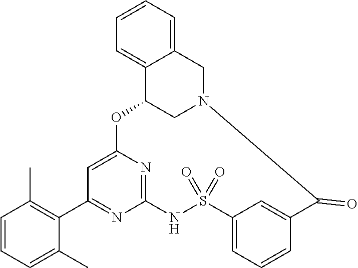 [SMILES: Cc1cccc(C)c1-c1cc2nc(n1)NS(=O)(=O)c1cccc(c1)C(=O)N1Cc3ccccc3[C@H](C1)O2] | ND | + | | |
| 198 | 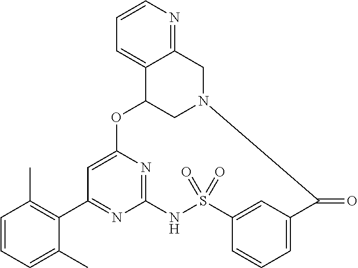 [SMILES: Cc1cccc(C)c1-c1cc2nc(n1)NS(=O)(=O)c1cccc(c1)C(=O)N1Cc3ncccc3C(C1)O2] | ++ | ++ | | |
| 2 | 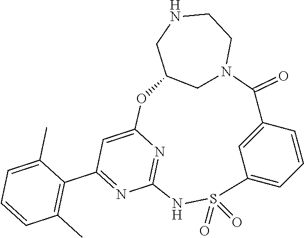 [SMILES: Cc1cccc(C)c1-c1cc2nc(n1)NS(=O)(=O)c1cccc(c1)C(=O)N1CCNC[C@H](C1)O2] | + | +++ | | |
| 197 | 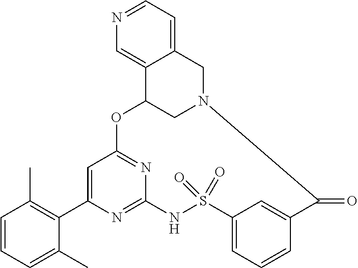 [SMILES: Cc1cccc(C)c1-c1cc2nc(n1)NS(=O)(=O)c1cccc(c1)C(=O)N1Cc3ccncc3C(C1)O2] | | | | |
| 231 | 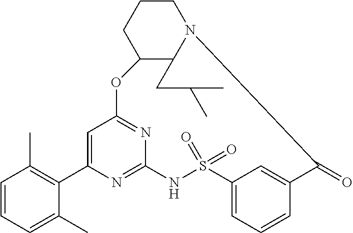 [SMILES: Cc1cccc(C)c1-c1cc2nc(n1)NS(=O)(=O)c1cccc(c1)C(=O)N1CCCC(O2)C1CC(C)C] | +++ | +++ | | |
| 196 | 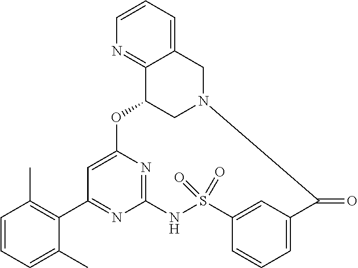 [SMILES: Cc1cccc(C)c1-c1cc2nc(n1)NS(=O)(=O)c1cccc(c1)C(=O)N1Cc3cccnc3[C@H](C1)O2] | ND | + | | |
| 195 | 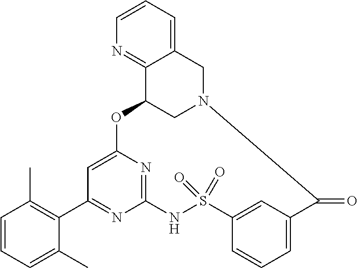 [SMILES: Cc1cccc(C)c1-c1cc2nc(n1)NS(=O)(=O)c1cccc(c1)C(=O)N1Cc3cccnc3[C@@H](C1)O2] | + | ++ | | |
| 35 | 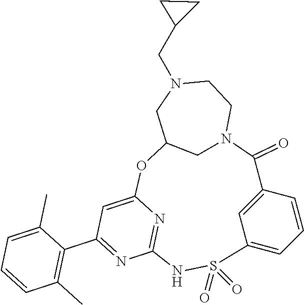 [SMILES: Cc1cccc(C)c1-c1cc2nc(n1)NS(=O)(=O)c1cccc(c1)C(=O)N1CCN(CC3CC3)CC(C1)O2] | +++ | +++ | | |
| 230 | 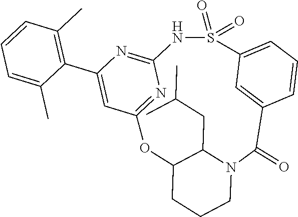 [SMILES: Cc1cccc(C)c1-c1cc2nc(n1)NS(=O)(=O)c1cccc(c1)C(=O)N1CCCC(O2)C1CC(C)C] | +++ | +++ | | |
| 199 | 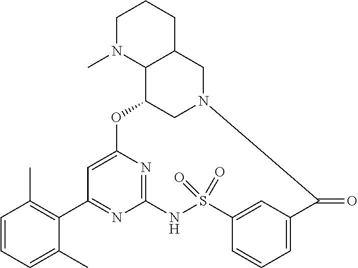 [SMILES: Cc1cccc(C)c1-c1cc2nc(n1)NS(=O)(=O)c1cccc(c1)C(=O)N1CC3CCCN(C)C3[C@H](C1)O2] | +++ | +++ | | |
| 200 | 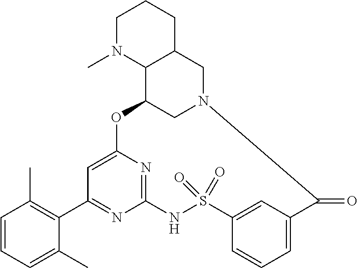 [SMILES: Cc1cccc(C)c1-c1cc2nc(n1)NS(=O)(=O)c1cccc(c1)C(=O)N1CC3CCCN(C)C3[C@@H](C1)O2] | ND | + | | |
| 194 | 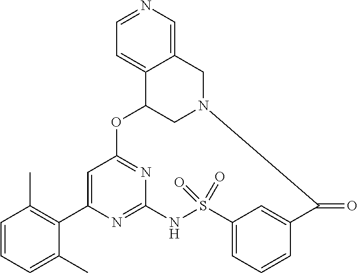 [SMILES: Cc1cccc(C)c1-c1cc2nc(n1)NS(=O)(=O)c1cccc(c1)C(=O)N1Cc3cnccc3C(C1)O2] | + | ++ | | |
| 209 | 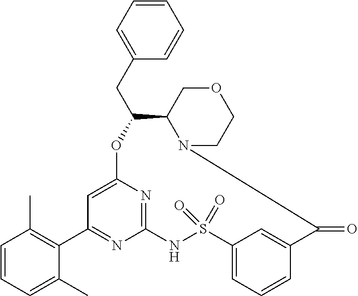 [SMILES: Cc1cccc(C)c1-c1cc2nc(n1)NS(=O)(=O)c1cccc(c1)C(=O)N1CCOC[C@@H]1[C@@H](Cc1ccccc1)O2] | +++ | +++ | | |
| 173 | 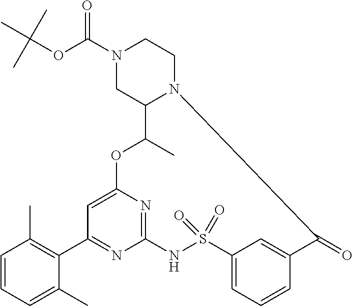 [SMILES: Cc1cccc(C)c1-c1cc2nc(n1)NS(=O)(=O)c1cccc(c1)C(=O)N1CCN(C(=O)OC(C)(C)C)CC1C(C)O2] | ++ | ++ | | |
| 172 | 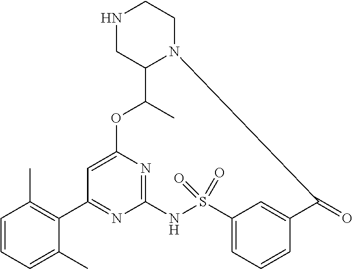 [SMILES: Cc1cccc(C)c1-c1cc2nc(n1)NS(=O)(=O)c1cccc(c1)C(=O)N1CCNCC1C(C)O2] | ND | + | | |
| 253 | 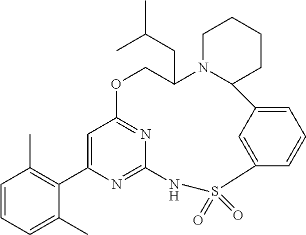 [SMILES: Cc1cccc(C)c1-c1cc2nc(n1)NS(=O)(=O)c1cccc(c1)C1CCCCN1C(CC(C)C)CO2] | ND | + | | |
| 340 | 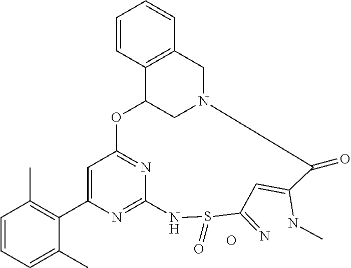 | + | + | | |
| 341 | 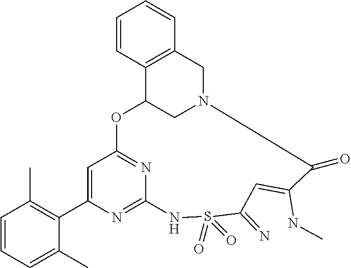 [SMILES: CN/C1=C\C(=N)S(=O)(=O)Nc2nc(cc(-c3c(C)cccc3C)n2)OC2CN(Cc3ccccc32)C1=O] | + | ++ | | |

The following table represent CFTR modulating activity for representative compounds of the disclosure generated using one or more of the assays disclosed herein (EC₅₀: +++ is <1 μM; ++ is 1-<3 μM; + is 3-<30 μM; and ND is “not detected in this assay.” % Activity: +++ is >600%; ++ is 30-60%; + is <30%).

TABLE 14

| | | Ent. | Ent. A | Ent. | Ent. B |
|---|---|---|---|---|---|
| | | A | Max | B | Max |
| Cmpd | | EC₅₀ | Activity | EC₅₀ | Activity |
| Number | Structure | (μM) | (%) | (μM) | (%) |
| 228 | 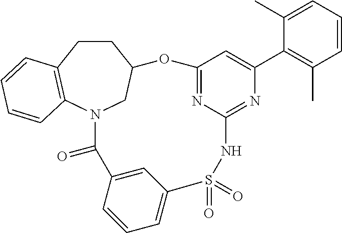 [SMILES: Cc1cccc(C)c1-c1cc2nc(n1)NS(=O)(=O)c1cccc(c1)C(=O)N1CC(CCc3ccccc31)O2] | ++ | ++ | | |
| 175 | 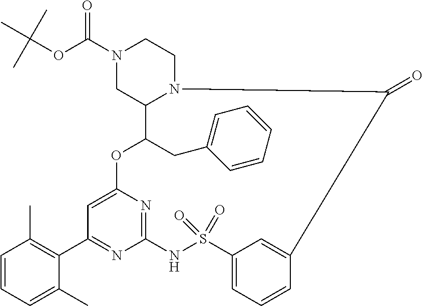 [SMILES: Cc1cccc(C)c1-c1cc2nc(n1)NS(=O)(=O)c1cccc(c1)C(=O)N1CCN(C(=O)OC(C)(C)C)CC1C(Cc1ccccc1)O2] | +++ | ++ | | |
| 176 | 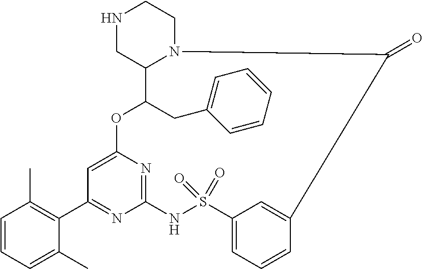 [SMILES: Cc1cccc(C)c1-c1cc2nc(n1)NS(=O)(=O)c1cccc(c1)C(=O)N1CCNCC1C(Cc1ccccc1)O2] | + | + | | |
| 174 | 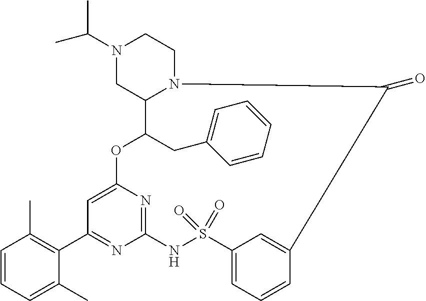 [SMILES: Cc1cccc(C)c1-c1cc2nc(n1)NS(=O)(=O)c1cccc(c1)C(=O)N1CCN(C(C)C)CC1C(Cc1ccccc1)O2] | +++ | +++ | | |
| 201 | 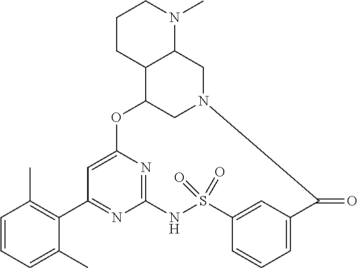 [SMILES: Cc1cccc(C)c1-c1cc2nc(n1)NS(=O)(=O)c1cccc(c1)C(=O)N1CC(O2)C2CCCN(C)C2C1] | + | ++ | | |
| 202 | 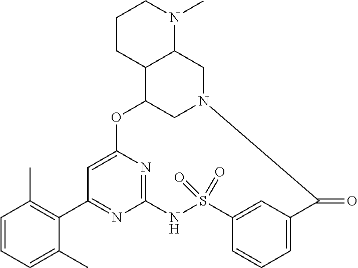 [SMILES: Cc1cccc(C)c1-c1cc2nc(n1)NS(=O)(=O)c1cccc(c1)C(=O)N1CC(O2)C2CCCN(C)C2C1] | ND | + | | |
| 94 | 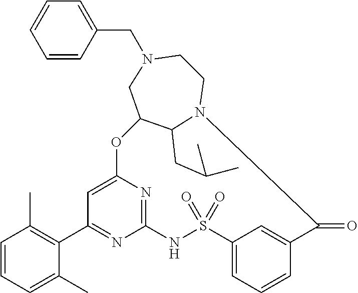 [SMILES: Cc1cccc(C)c1-c1cc2nc(n1)NS(=O)(=O)c1cccc(c1)C(=O)N1CCN(Cc3ccccc3)CC(O2)C1CC(C)C] | +++ | +++ | | |
| 90 | 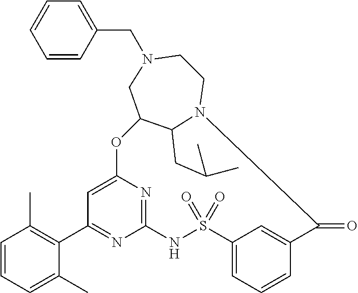 [SMILES: Cc1cccc(C)c1-c1cc2nc(n1)NS(=O)(=O)c1cccc(c1)C(=O)N1CCN(Cc3ccccc3)CC(O2)C1CC(C)C] | +++ | +++ | | |
| 93 | 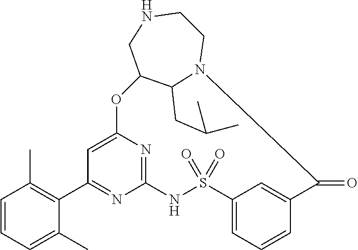 [SMILES: Cc1cccc(C)c1-c1cc2nc(n1)NS(=O)(=O)c1cccc(c1)C(=O)N1CCNCC(O2)C1CC(C)C] | ++ | +++ | | |
| 91 | 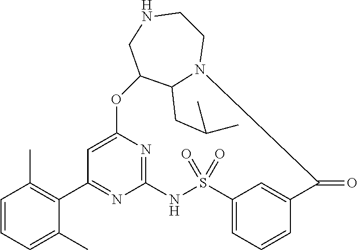 [SMILES: Cc1cccc(C)c1-c1cc2nc(n1)NS(=O)(=O)c1cccc(c1)C(=O)N1CCNCC(O2)C1CC(C)C] | ++ | +++ | | |
| 69 | 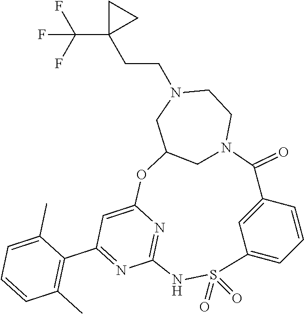 [SMILES: Cc1cccc(C)c1-c1cc2nc(n1)NS(=O)(=O)c1cccc(c1)C(=O)N1CCN(CCC3(C(F)(F)F)CC3)CC(C1)O2] | +++ | +++ | | |
| 251 | 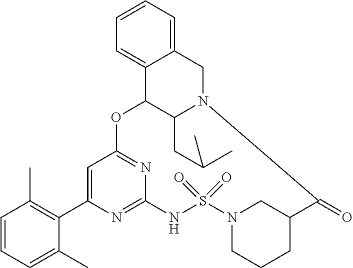 [SMILES: Cc1cccc(C)c1-c1cc2nc(n1)NS(=O)(=O)N1CCCC(C1)C(=O)N1Cc3ccccc3C(O2)C1CC(C)C] | ND | ND | | |
| 203 | 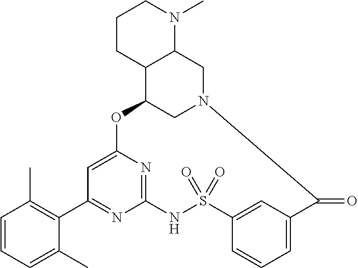 [SMILES: Cc1cccc(C)c1-c1cc2nc(n1)NS(=O)(=O)c1cccc(c1)C(=O)N1CC3C(CCCN3C)[C@@H](C1)O2] | + | ++ | | |
| 204 | 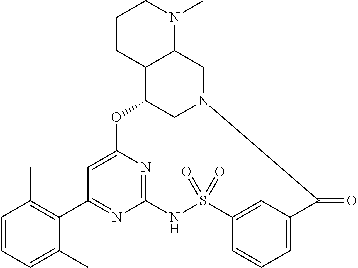 [SMILES: Cc1cccc(C)c1-c1cc2nc(n1)NS(=O)(=O)c1cccc(c1)C(=O)N1CC3C(CCCN3C)[C@H](C1)O2] | ++ | +++ | | |
| 92 | 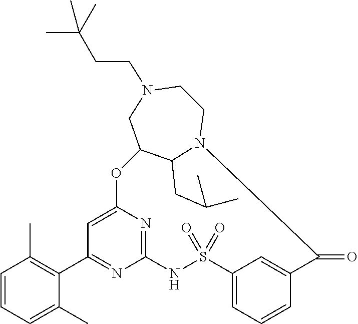 [SMILES: Cc1cccc(C)c1-c1cc2nc(n1)NS(=O)(=O)c1cccc(c1)C(=O)N1CCN(CCC(C)(C)C)CC(O2)C1CC(C)C] | +++ | +++ | | |
| 88 | 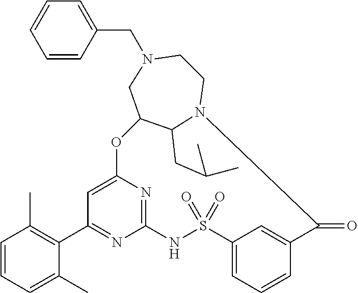 [SMILES: Cc1cccc(C)c1-c1cc2nc(n1)NS(=O)(=O)c1cccc(c1)C(=O)N1CCN(Cc3ccccc3)CC(O2)C1CC(C)C] | +++ | ++ | | |
| 89 | 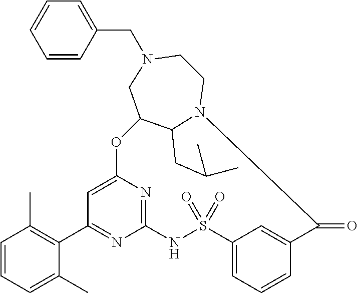 [SMILES: Cc1cccc(C)c1-c1cc2nc(n1)NS(=O)(=O)c1cccc(c1)C(=O)N1CCN(Cc3ccccc3)CC(O2)C1CC(C)C] | +++ | +++ | | |
| 193 | 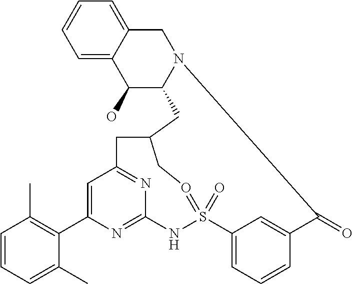 | +++ | +++ | | |
| 192 | 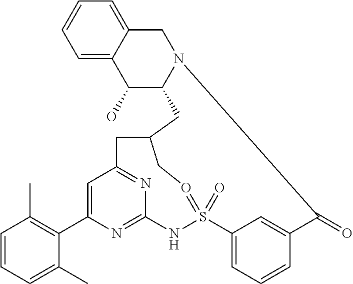 | +++ | +++ | | |
| 70 | 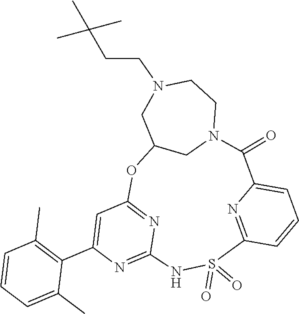 [SMILES: Cc1cccc(C)c1-c1cc2nc(n1)NS(=O)(=O)c1cccc(n1)C(=O)N1CCN(CCC(C)(C)C)CC(C1)O2] | ND | + | | |
| 249 | 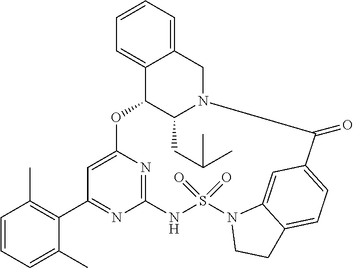 [SMILES: Cc1cccc(C)c1-c1cc2nc(n1)NS(=O)(=O)N1CCc3ccc(cc31)C(=O)N1Cc3ccccc3[C@@H](O2)[C@H]1CC(C)C] | +++ | ++ | | |
| 250 | 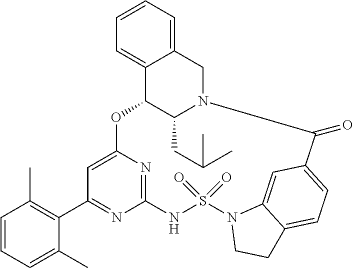 [SMILES: Cc1cccc(C)c1-c1cc2nc(n1)NS(=O)(=O)N1CCc3ccc(cc31)C(=O)N1Cc3ccccc3[C@@H](O2)[C@H]1CC(C)C] | ++ | ++ | | |
| 355 | 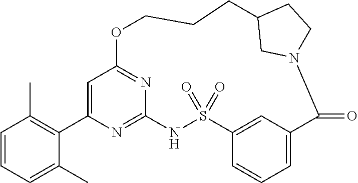 [SMILES: Cc1cccc(C)c1-c1cc2nc(n1)NS(=O)(=O)c1cccc(c1)C(=O)N1CCC(CCCO2)C1] | + | ++ | | |
| 356 | 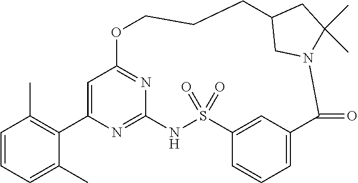 [SMILES: Cc1cccc(C)c1-c1cc2nc(n1)NS(=O)(=O)c1cccc(c1)C(=O)N1CC(CCCO2)CC1(C)C] | + | ++ | | |
| 243 | 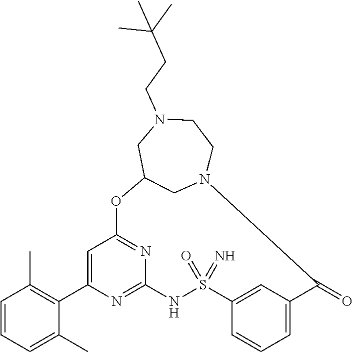 [SMILES: Cc1cccc(C)c1-c1cc2nc(n1)NS(=N)(=O)c1cccc(c1)C(=O)N1CCN(CCC(C)(C)C)CC(C1)O2] | +++ | +++ | +++ | +++ |
| 239 | 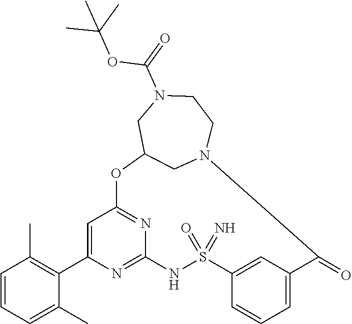 [SMILES: Cc1cccc(C)c1-c1cc2nc(n1)NS(=N)(=O)c1cccc(c1)C(=O)N1CCN(C(=O)OC(C)(C)C)CC(C1)O2] | ++ | +++ | ++ | +++ |
| 240 | 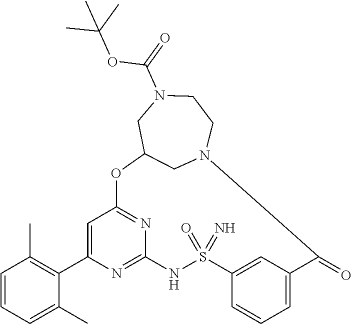 [SMILES: Cc1cccc(C)c1-c1cc2nc(n1)NS(=N)(=O)c1cccc(c1)C(=O)N1CCN(C(=O)OC(C)(C)C)CC(C1)O2] | + | ++ | | |
| 241 | 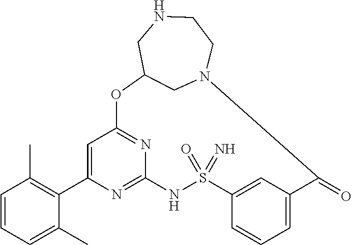 [SMILES: Cc1cccc(C)c1-c1cc2nc(n1)NS(=N)(=O)c1cccc(c1)C(=O)N1CCNCC(C1)O2] | ND | + | | |
| 242 | 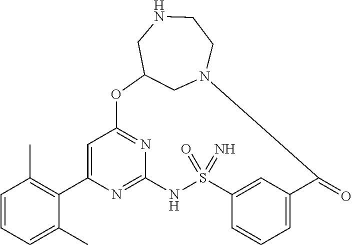 [SMILES: Cc1cccc(C)c1-c1cc2nc(n1)NS(=N)(=O)c1cccc(c1)C(=O)N1CCNCC(C1)O2] | ND | + | | |
| 222 | 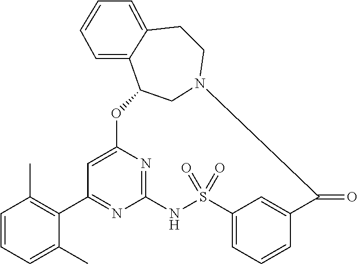 [SMILES: Cc1cccc(C)c1-c1cc2nc(n1)NS(=O)(=O)c1cccc(c1)C(=O)N1CCc3ccccc3[C@H](C1)O2] | | | | |
| 221 | 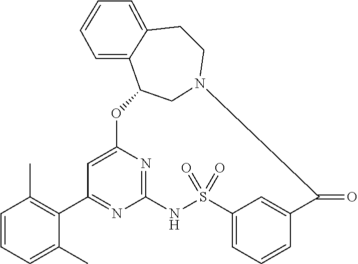 [SMILES: Cc1cccc(C)c1-c1cc2nc(n1)NS(=O)(=O)c1cccc(c1)C(=O)N1CCc3ccccc3[C@H](C1)O2] | +++ | +++ | | |
| 244 | 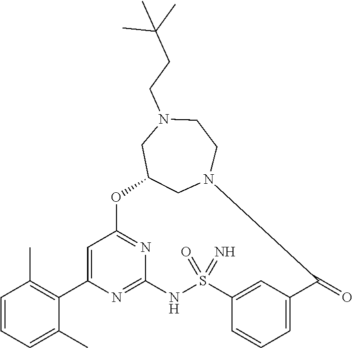 [SMILES: Cc1cccc(C)c1-c1cc2nc(n1)NS(=N)(=O)c1cccc(c1)C(=O)N1CCN(CCC(C)(C)C)C[C@H](C1)O2] | ND | + | | |
| 245 | 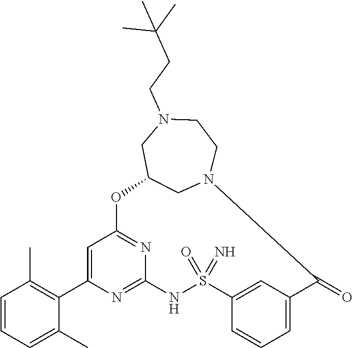 [SMILES: Cc1cccc(C)c1-c1cc2nc(n1)NS(=N)(=O)c1cccc(c1)C(=O)N1CCN(CCC(C)(C)C)C[C@H](C1)O2] | +++ | ++ | +++ | +++ |
| 232 | 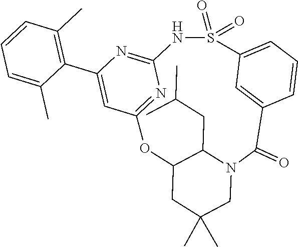 [SMILES: Cc1cccc(C)c1-c1cc2nc(n1)NS(=O)(=O)c1cccc(c1)C(=O)N1CC(C)(C)CC(O2)C1CC(C)C] | +++ | +++ | | |
| 248 | 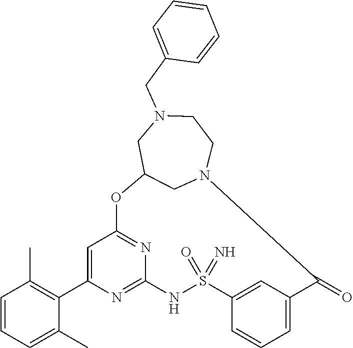 [SMILES: Cc1cccc(C)c1-c1cc2nc(n1)NS(=N)(=O)c1cccc(c1)C(=O)N1CCN(Cc3ccccc3)CC(C1)O2] | ++ | ++ | +++ | +++ |
| 84 | 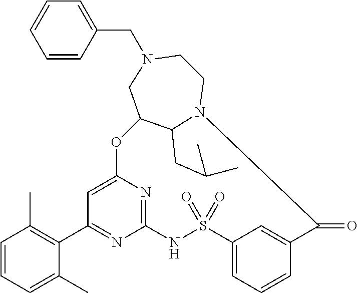 [SMILES: Cc1cccc(C)c1-c1cc2nc(n1)NS(=O)(=O)c1cccc(c1)C(=O)N1CCN(Cc3ccccc3)CC(O2)C1CC(C)C] | +++ | +++ | | |
| 87 | 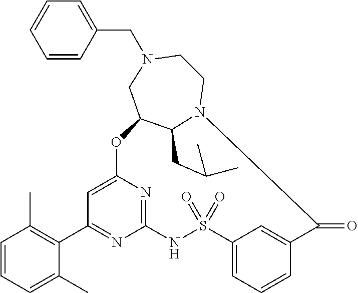 [SMILES: Cc1cccc(C)c1-c1cc2nc(n1)NS(=O)(=O)c1cccc(c1)C(=O)N1CCN(Cc3ccccc3)C[C@H](O2)[C@@H]1CC(C)C] | +++ | ++ | | |
| 247 | 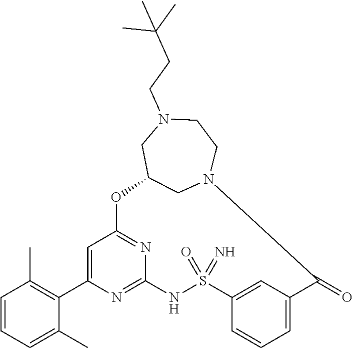 [SMILES: Cc1cccc(C)c1-c1cc2nc(n1)NS(=N)(=O)c1cccc(c1)C(=O)N1CCN(CCC(C)(C)C)C[C@H](C1)O2] | ++ | + | | |
| 246 | 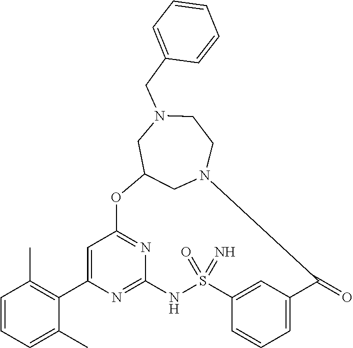 [SMILES: Cc1cccc(C)c1-c1cc2nc(n1)NS(=N)(=O)c1cccc(c1)C(=O)N1CCN(Cc3ccccc3)CC(C1)O2] | ++ | ++ | | |
| 233 | 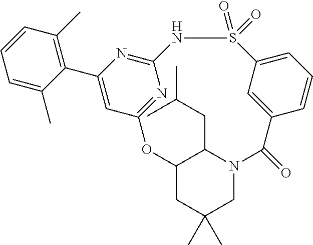 [SMILES: Cc1cccc(C)c1-c1cc2nc(n1)NS(=O)(=O)c1cccc(c1)C(=O)N1CC(C)(C)CC(O2)C1CC(C)C] | +++ | +++ | | |
| 19 | 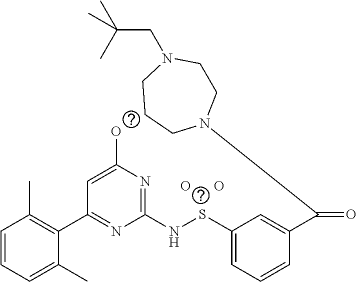 | +++ | +++ | | |
| 18 | 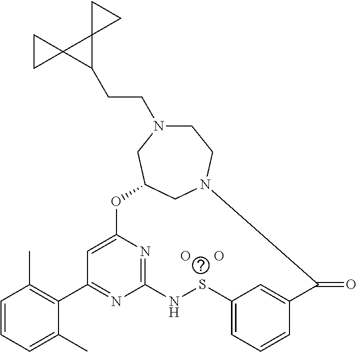 | ++ | + | | |
| 17 | 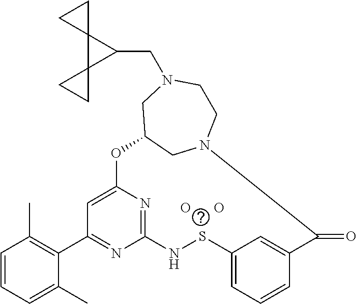 | +++ | +++ | | |
| 16 | 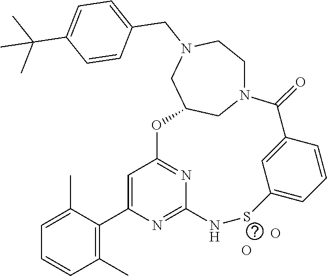 | +++ | +++ | | |
| 5 | 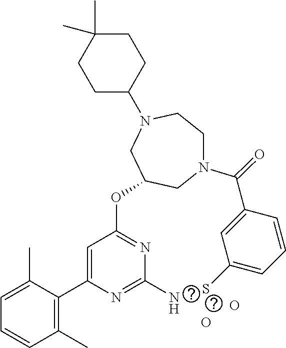 | +++ | +++ | | |
| 74 | 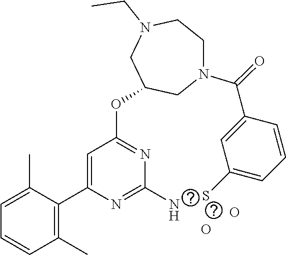 | ++ | +++ | | |
| 359 | 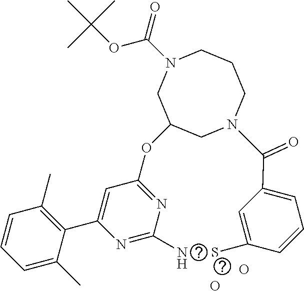 | ++ | ++ | | |
| 206 | 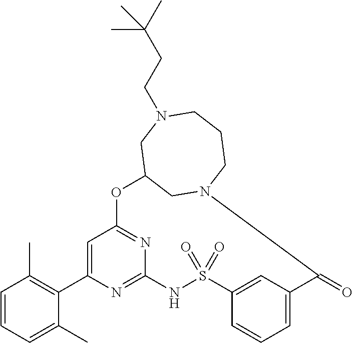 [SMILES: Cc1cccc(C)c1-c1cc2nc(n1)NS(=O)(=O)c1cccc(c1)C(=O)N1CCCN(CCC(C)(C)C)CC(C1)O2] | +++ | ++ | | |
| 205 | 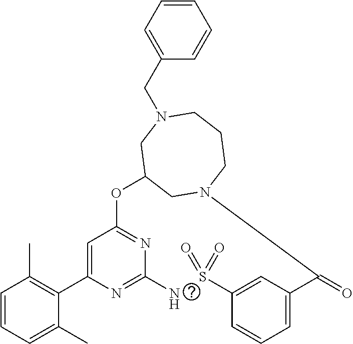 | +++ | ++ | | |
| 358 | 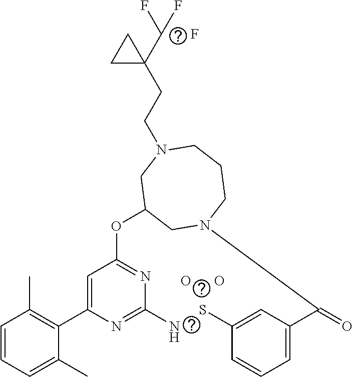 | +++ | ++ | | |
| 357 | 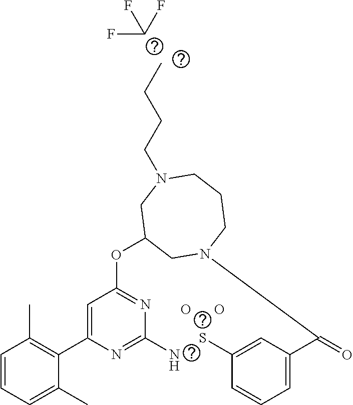 | +++ | ++ | | |
| 75 | 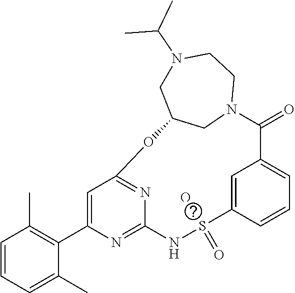 | +++ | +++ | | |
| 34 | 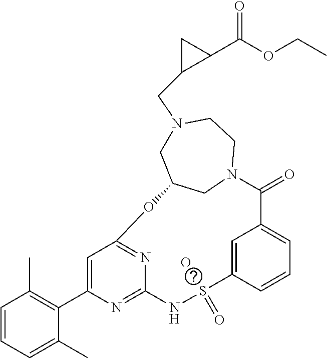 | +++ | +++ | | |
| 33 | 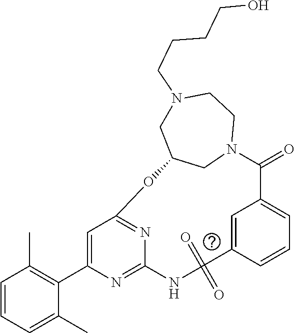 | + | +++ | | |
| 31 | 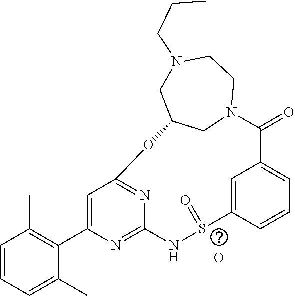 | +++ | +++ | | |
| 30 | 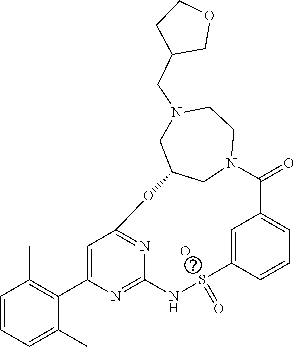 | ++ | +++ | | |
| 29 | 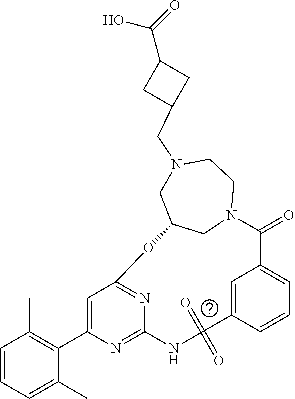 | + | +++ | | |
| 6 | 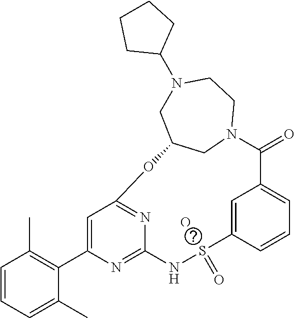 | +++ | +++ | | |
| 28 | 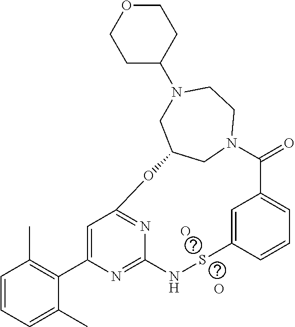 | + | +++ | | |
| 4 | 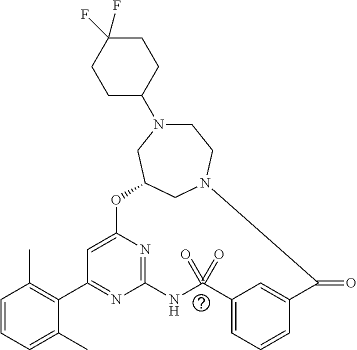 | +++ | +++ | | |
| 32 | 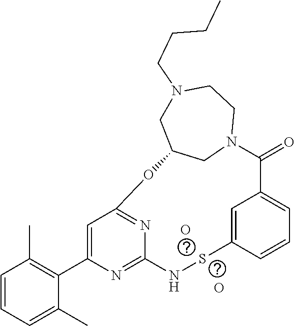 | +++ | +++ | | |
| 362 | 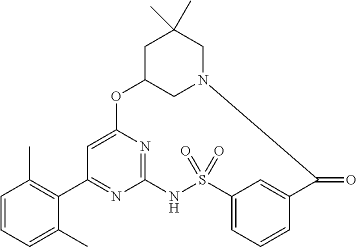 [SMILES: Cc1cccc(C)c1-c1cc2nc(n1)NS(=O)(=O)c1cccc(c1)C(=O)N1CC(CC(C)(C)C1)O2] | +++ | ++ | | |
| 182 | 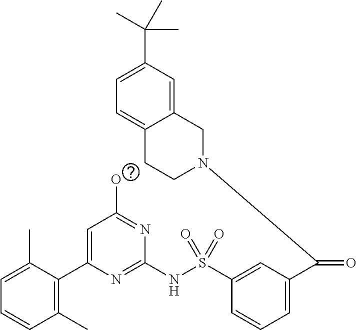 [SMILES: Cc1cccc(C)c1-c1cc(O)nc(NS(=O)(=O)c2cccc(C(=O)N3CCc4ccc(C(C)(C)C)cc4C3)c2)n1] | ND | + | | |
| 183 | 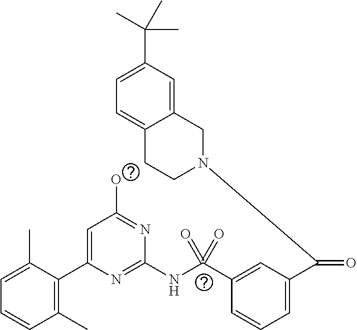 | +++ | +++ | +++ | +++ |
| 86 | 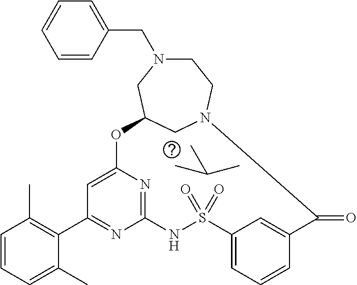 | +++ | +++ | | |
| 76 | 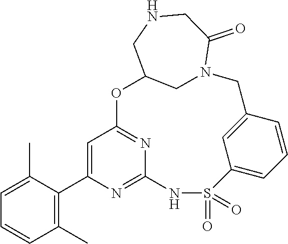 [SMILES: Cc1cccc(C)c1-c1cc2nc(n1)NS(=O)(=O)c1cccc(c1)CN1CC(CNCC1=O)O2] | ND | + | | |
| 78 | 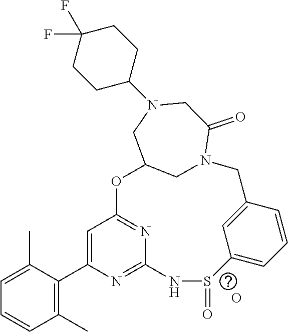 | +++ | ++ | | |
| 145 | 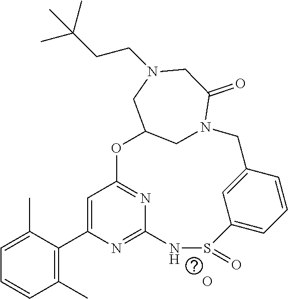 | +++ | ++ | | |
| 144 | 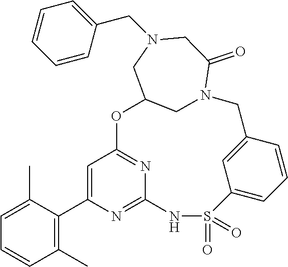 [SMILES: Cc1cccc(C)c1-c1cc2nc(n1)NS(=O)(=O)c1cccc(c1)CN1CC(CN(Cc3ccccc3)CC1=O)O2] | +++ | ++ | | |
| 77 | 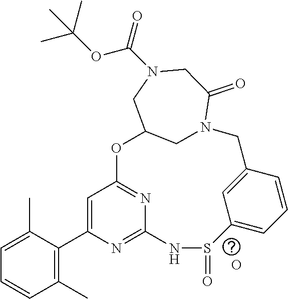 | +++ | ++ | | |
| 184 | 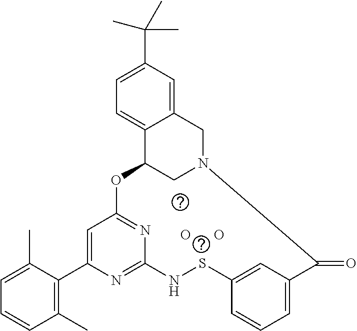 | +++ | +++ | | |
| 185 | 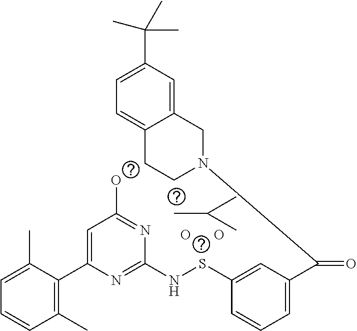 | +++ | +++ | | |
| 27 | 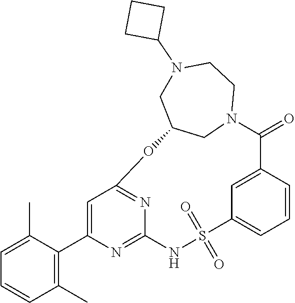 [SMILES: Cc1cccc(C)c1-c1cc2nc(n1)NS(=O)(=O)c1cccc(c1)C(=O)N1CCN(C3CCC3)C[C@H](C1)O2] | +++ | ++ | | |
| 26 | 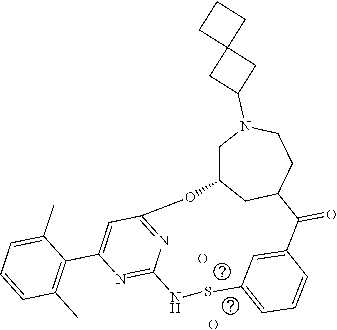 | +++ | +++ | | |
| 25 | 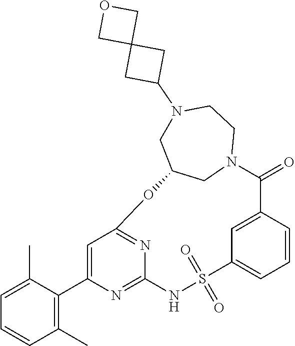 [SMILES: Cc1cccc(C)c1-c1cc2nc(n1)NS(=O)(=O)c1cccc(c1)C(=O)N1CCN(C3CC4(COC4)C3)C[C@H](C1)O2] | + | ++ | | |
| 24 | 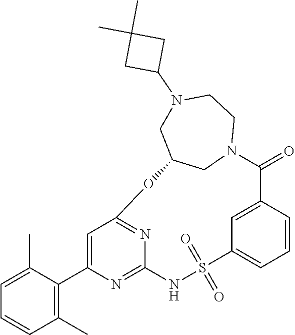 [SMILES: Cc1cccc(C)c1-c1cc2nc(n1)NS(=O)(=O)c1cccc(c1)C(=O)N1CCN(C3CC(C)(C)C3)C[C@H](C1)O2] | +++ | ++ | | |
| 8 | 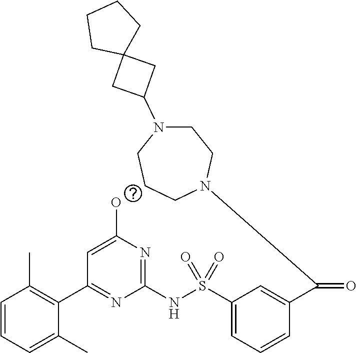 [SMILES: Cc1cccc(C)c1-c1cc(O)nc(NS(=O)(=O)c2cccc(C(=O)N3CCCN(C4CC5(CCCC5)C4)CC3)c2)n1] | +++ | +++ | | |
| 3 | 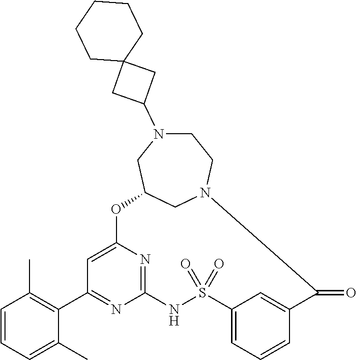 [SMILES: Cc1cccc(C)c1-c1cc2nc(n1)NS(=O)(=O)c1cccc(c1)C(=O)N1CCN(C3CC4(CCCCC4)C3)C[C@H](C1)O2] | +++ | +++ | | |
| 23 | 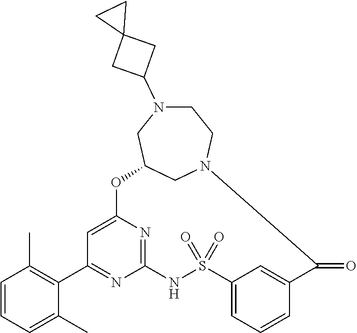 [SMILES: Cc1cccc(C)c1-c1cc2nc(n1)NS(=O)(=O)c1cccc(c1)C(=O)N1CCN(C3CC4(CC4)C3)C[C@H](C1)O2] | +++ | +++ | | |
| 210 | 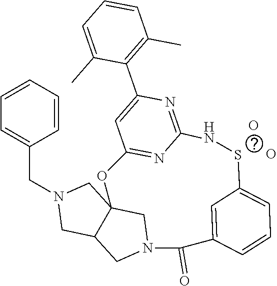 | +++ | +++ | | |
| 82 | 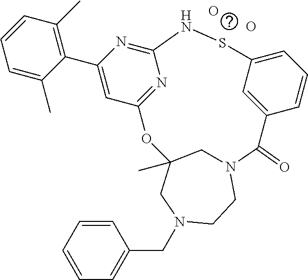 | ++ | +++ | | |
| 81 | 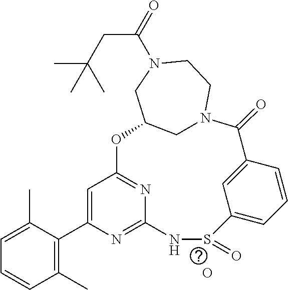 | ++ | +++ | | |
| 80 | 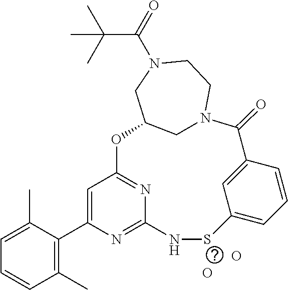 | + | +++ | | |
| 79 | 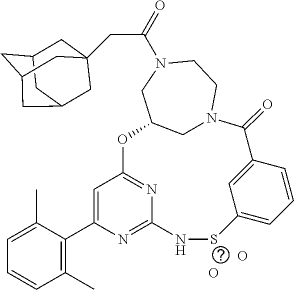 | +++ | +++ | | |
| 22 | 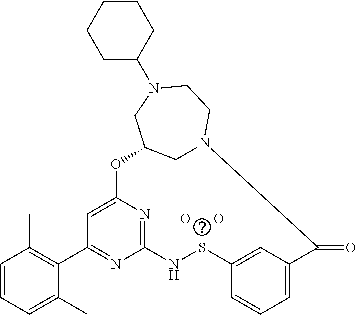 | +++ | +++ | | |
| 11 | 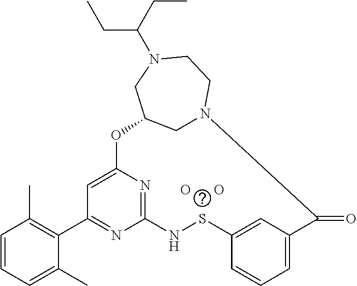 | +++ | +++ | | |
| 159 | 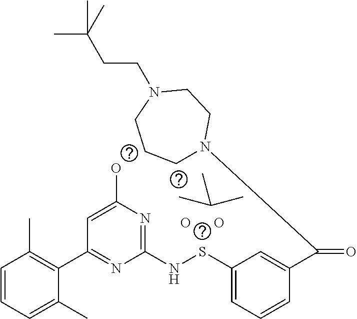 | +++ | +++ | | |
| 158 | 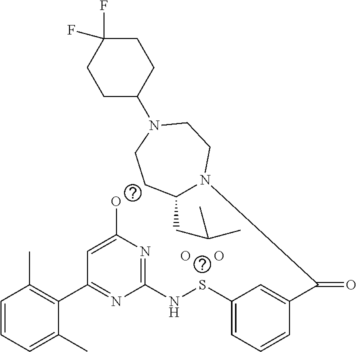 | +++ | +++ | | |
| 157 | 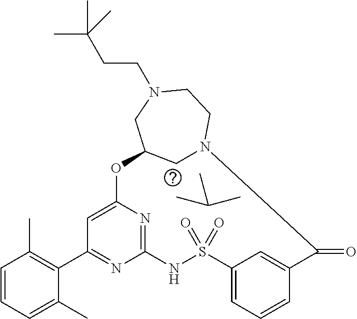 | +++ | +++ | | |
| 85 | 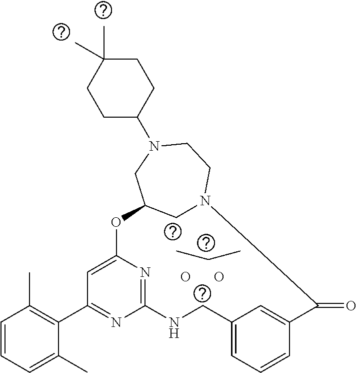 | +++ | +++ | | |
| 20 | 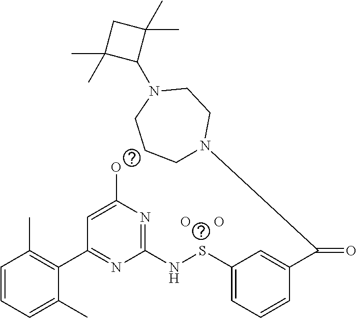 | +++ | +++ | | |
| 21 | 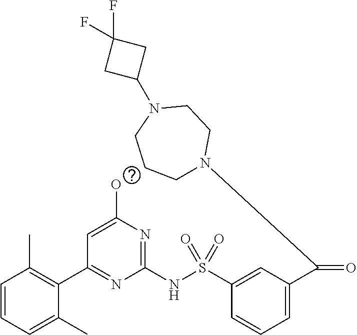 [SMILES: Cc1cccc(C)c1-c1cc(O)nc(NS(=O)(=O)c2cccc(C(=O)N3CCCN(C4CC(F)(F)C4)CC3)c2)n1] | +++ | +++ | | |
| 9 | 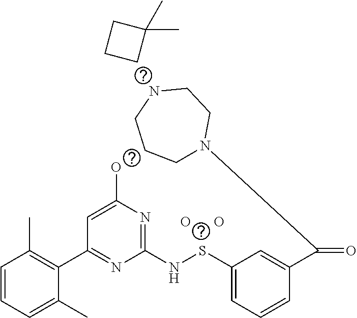 | +++ | +++ | | |
| 10 | 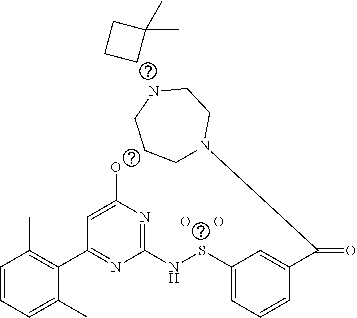 | +++ | +++ | | |
| 36 | 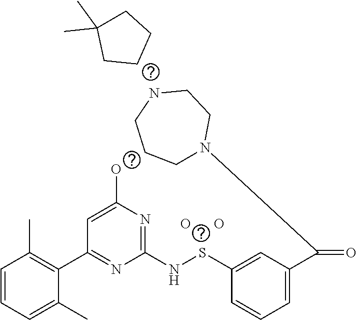 | +++ | +++ | | |
| 37 | 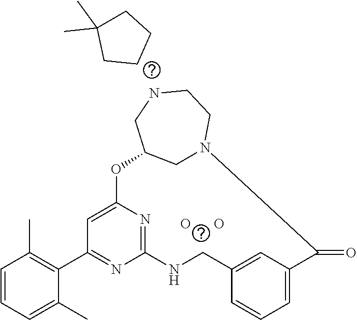 | +++ | +++ | | |
| 109 | 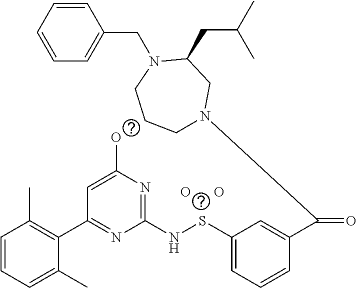 | +++ | +++ | | |
| 107 | 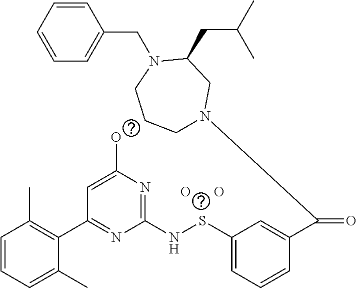 | +++ | +++ | | |
| 46 | 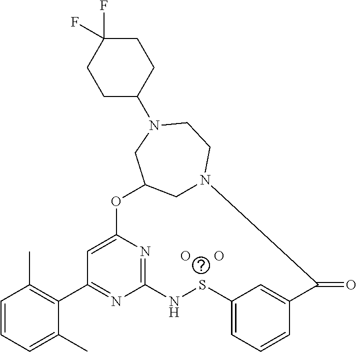 | +++ | +++ | | |
| 100 | 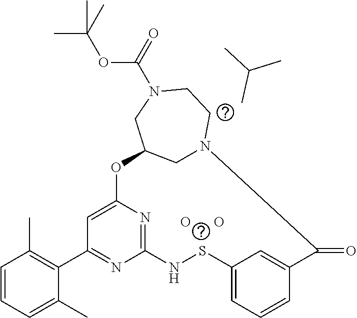 | ND | + | | |
| 96 | 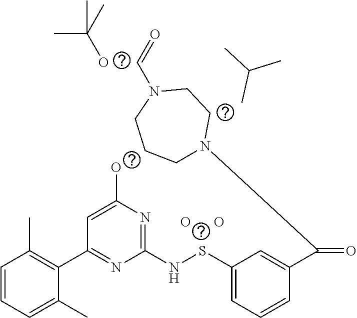 | +++ | ++ | | |
| 83 | 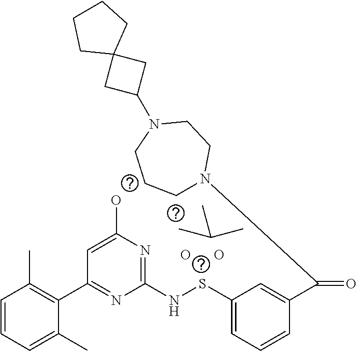 | +++ | +++ | | |
| 186 | 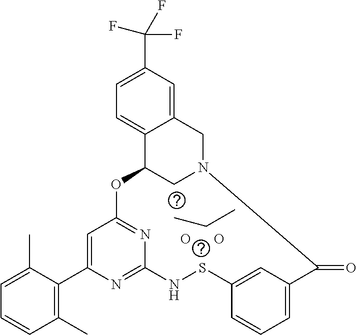 | +++ | +++ | +++ | +++ |
| 187 | 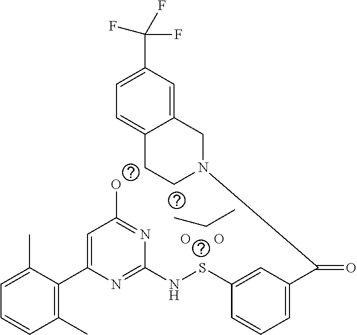 | +++ | +++ | | |
| 108 | 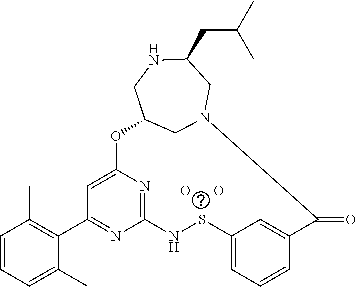 | +++ | +++ | | |
| 110 | 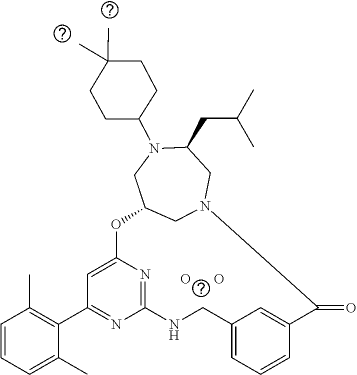 | +++ | +++ | | |
| 156 | 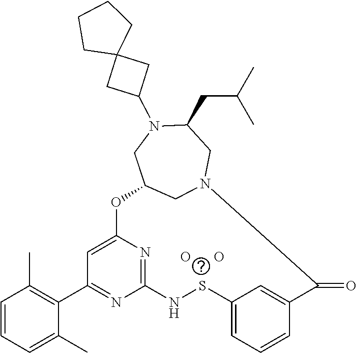 | +++ | +++ | | |
| 155 | 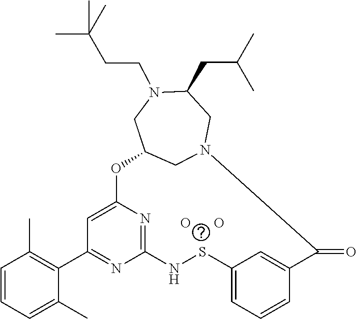 | +++ | +++ | | |
| 111 | 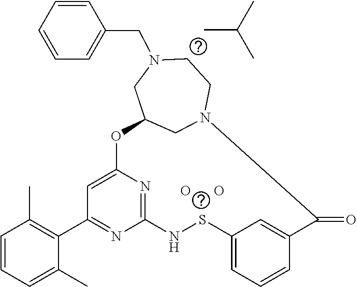 | ND | + | | |
| 106 | 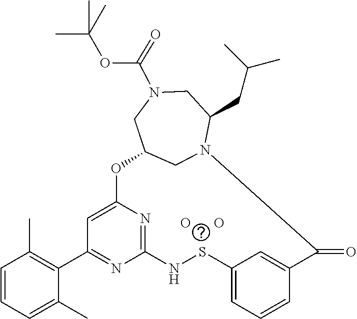 | ND | + | | |
| 102 | 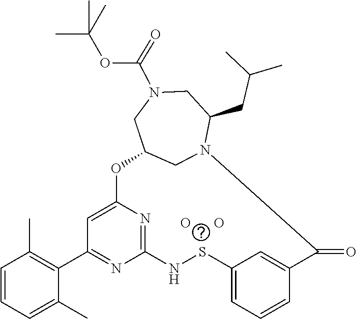 | +++ | ++ | | |
| 15 | 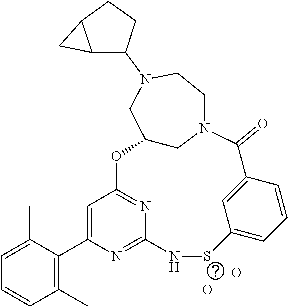 | +++ | +++ | | |
| 40 | 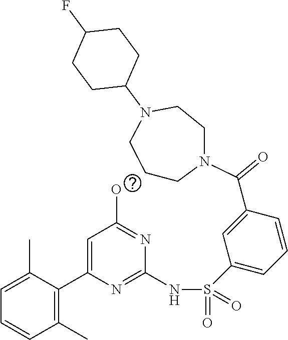 [SMILES: Cc1cccc(C)c1-c1cc(O)nc(NS(=O)(=O)c2cccc(C(=O)N3CCCN(C4CCC(F)CC4)CC3)c2)n1] | +++ | +++ | | |
| 154 | 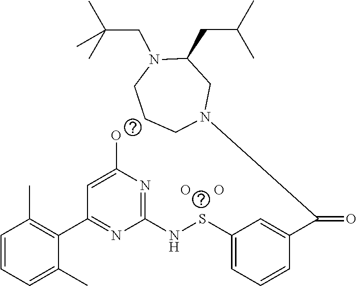 | +++ | +++ | | |
| 95 | 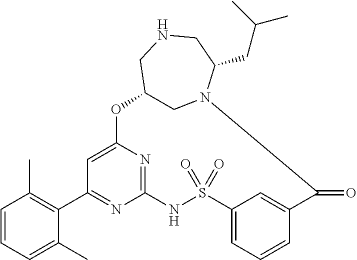 [SMILES: Cc1cccc(C)c1-c1cc2nc(n1)NS(=O)(=O)c1cccc(c1)C(=O)N1C[C@@H](CNC[C@@H]1CC(C)C)O2] | +++ | ++ | | |
| 97 | 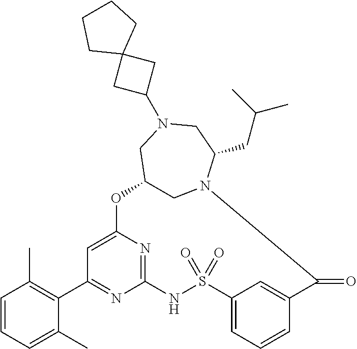 [SMILES: Cc1cccc(C)c1-c1cc2nc(n1)NS(=O)(=O)c1cccc(c1)C(=O)N1C[C@@H](CN(C3CC4(CCCC4)C3)C[C@@H]1CC(C)C)O2] | +++ | ++ | | |
| 153 | 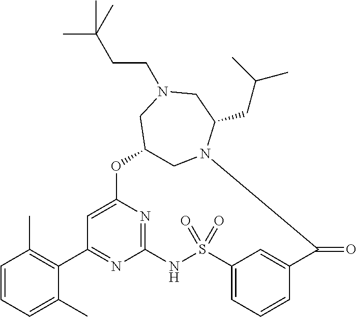 [SMILES: Cc1cccc(C)c1-c1cc2nc(n1)NS(=O)(=O)c1cccc(c1)C(=O)N1C[C@@H](CN(CCC(C)(C)C)C[C@@H]1CC(C)C)O2] | +++ | ++ | | |
| 152 | 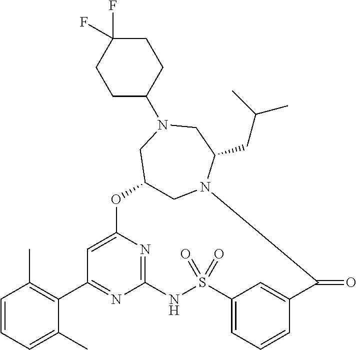 [SMILES: Cc1cccc(C)c1-c1cc2nc(n1)NS(=O)(=O)c1cccc(c1)C(=O)N1C[C@@H](CN(C3CCC(F)(F)CC3)C[C@@H]1CC(C)C)O2] | +++ | ++ | | |
| 151 | 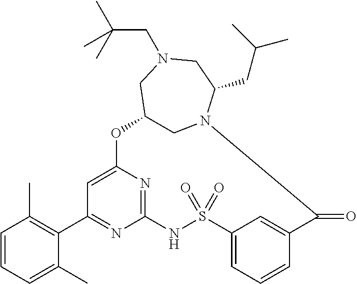 [SMILES: Cc1cccc(C)c1-c1cc2nc(n1)NS(=O)(=O)c1cccc(c1)C(=O)N1C[C@@H](CN(CC(C)(C)C)C[C@@H]1CC(C)C)O2] | +++ | ++ | | |
| 72 | 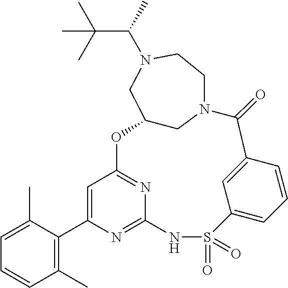 [SMILES: Cc1cccc(C)c1-c1cc2nc(n1)NS(=O)(=O)c1cccc(c1)C(=O)N1CCN([C@@H](C)C(C)(C)C)C[C@H](C1)O2] | +++ | +++ | | |
| 73 | 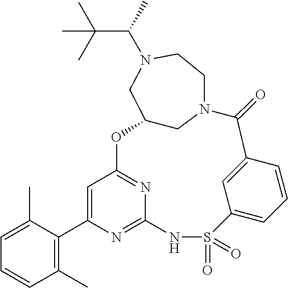 [SMILES: Cc1cccc(C)c1-c1cc2nc(n1)NS(=O)(=O)c1cccc(c1)C(=O)N1CCN([C@@H](C)C(C)(C)C)C[C@H](C1)O2] | +++ | +++ | | |
| 14 | 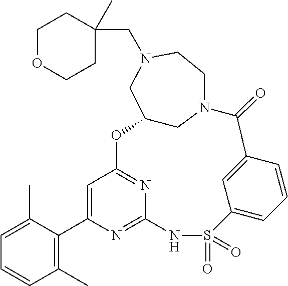 [SMILES: Cc1cccc(C)c1-c1cc2nc(n1)NS(=O)(=O)c1cccc(c1)C(=O)N1CCN(CC3(C)CCOCC3)C[C@H](C1)O2] | +++ | +++ | | |
| 188 | 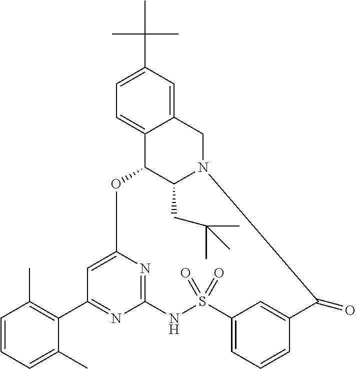 [SMILES: Cc1cccc(C)c1-c1cc2nc(n1)NS(=O)(=O)c1cccc(c1)C(=O)N1Cc3cc(C(C)(C)C)ccc3[C@@H](O2)[C@H]1CC(C)(C)C] | +++ | +++ | +++ | +++ |
| 189 | 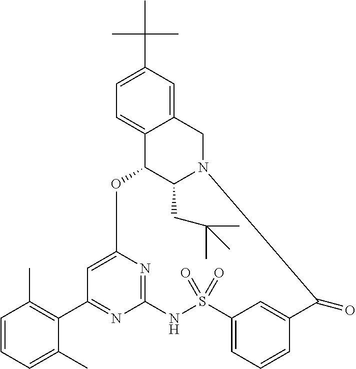 [SMILES: Cc1cccc(C)c1-c1cc2nc(n1)NS(=O)(=O)c1cccc(c1)C(=O)N1Cc3cc(C(C)(C)C)ccc3[C@@H](O2)[C@H]1CC(C)(C)C] | +++ | +++ | +++ | +++ |
| 104 | 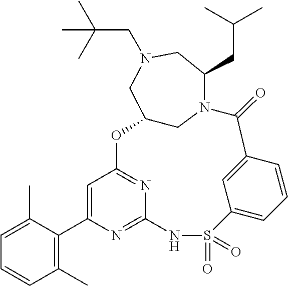 [SMILES: Cc1cccc(C)c1-c1cc2nc(n1)NS(=O)(=O)c1cccc(c1)C(=O)N1C[C@@H](CN(CC(C)(C)C)C[C@H]1CC(C)C)O2] | +++ | +++ | | |
| 143 | 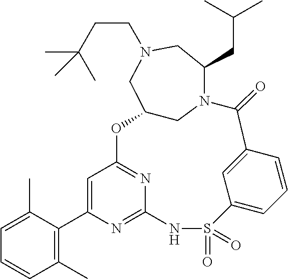 [SMILES: Cc1cccc(C)c1-c1cc2nc(n1)NS(=O)(=O)c1cccc(c1)C(=O)N1C[C@@H](CN(CCC(C)(C)C)C[C@H]1CC(C)C)O2] | +++ | +++ | | |
| 142 | 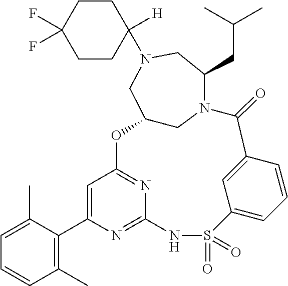 [SMILES: Cc1cccc(C)c1-c1cc2nc(n1)NS(=O)(=O)c1cccc(c1)C(=O)N1C[C@@H](CN(C3CCC(F)(F)CC3)C[C@H]1CC(C)C)O2] | +++ | +++ | | |
| 141 | 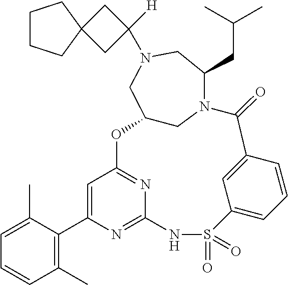 [SMILES: Cc1cccc(C)c1-c1cc2nc(n1)NS(=O)(=O)c1cccc(c1)C(=O)N1C[C@@H](CN(C3CC4(CCCC4)C3)C[C@H]1CC(C)C)O2] | +++ | +++ | | |
| 140 | 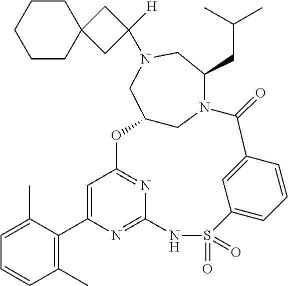 [SMILES: Cc1cccc(C)c1-c1cc2nc(n1)NS(=O)(=O)c1cccc(c1)C(=O)N1C[C@@H](CN(C3CC4(CCCCC4)C3)C[C@H]1CC(C)C)O2] | +++ | +++ | | |
| 139 | 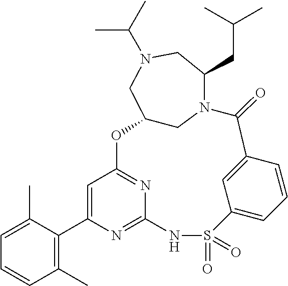 [SMILES: Cc1cccc(C)c1-c1cc2nc(n1)NS(=O)(=O)c1cccc(c1)C(=O)N1C[C@@H](CN(C(C)C)C[C@H]1CC(C)C)O2] | +++ | +++ | | |
| 138 | 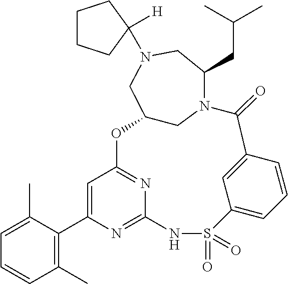 [SMILES: Cc1cccc(C)c1-c1cc2nc(n1)NS(=O)(=O)c1cccc(c1)C(=O)N1C[C@@H](CN(C3CCCC3)C[C@H]1CC(C)C)O2] | +++ | ++ | | |
| 101 | 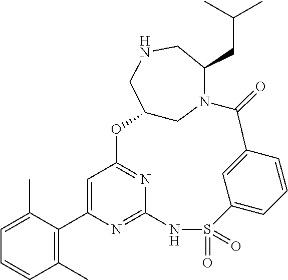 [SMILES: Cc1cccc(C)c1-c1cc2nc(n1)NS(=O)(=O)c1cccc(c1)C(=O)N1C[C@@H](CNC[C@H]1CC(C)C)O2] | +++ | ++ | | |
| 112 | 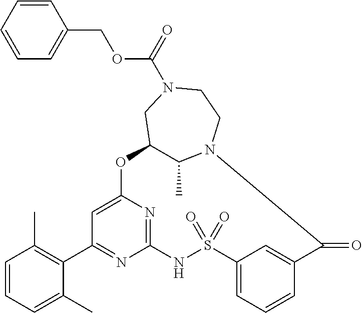 [SMILES: Cc1cccc(C)c1-c1cc2nc(n1)NS(=O)(=O)c1cccc(c1)C(=O)N1CCN(C(=O)OCc3ccccc3)C[C@H](O2)[C@H]1C] | +++ | +++ | | |
| 113 | 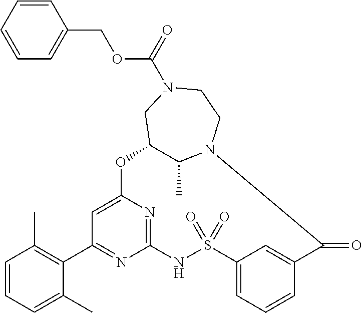 [SMILES: Cc1cccc(C)c1-c1cc2nc(n1)NS(=O)(=O)c1cccc(c1)C(=O)N1CCN(C(=O)OCc3ccccc3)C[C@@H](O2)[C@H]1C] | + | + | | |
| 105 | 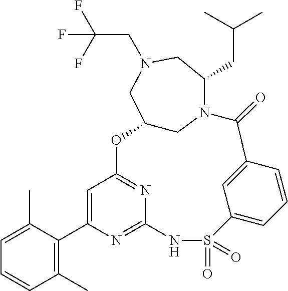 [SMILES: Cc1cccc(C)c1-c1cc2nc(n1)NS(=O)(=O)c1cccc(c1)C(=O)N1C[C@@H](CN(CC(F)(F)F)C[C@@H]1CC(C)C)O2] | ND | + | | |
| 114 | 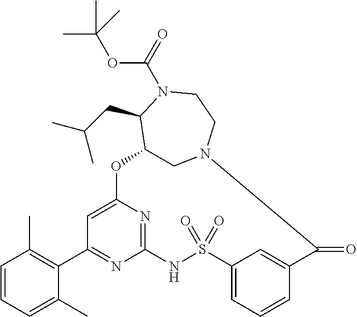 [SMILES: Cc1cccc(C)c1-c1cc2nc(n1)NS(=O)(=O)c1cccc(c1)C(=O)N1CCN(C(=O)OC(C)(C)C)[C@H](CC(C)C)[C@H](C1)O2] | +++ | +++ | | |
| 115 | 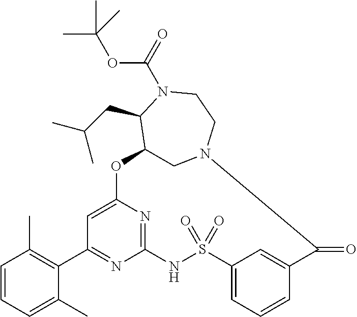 [SMILES: Cc1cccc(C)c1-c1cc2nc(n1)NS(=O)(=O)c1cccc(c1)C(=O)N1CCN(C(=O)OC(C)(C)C)[C@H](CC(C)C)[C@@H](C1)O2] | +++ | ++ | | |
| 150 | 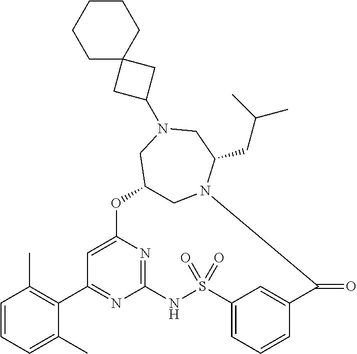 [SMILES: Cc1cccc(C)c1-c1cc2nc(n1)NS(=O)(=O)c1cccc(c1)C(=O)N1C[C@@H](CN(C3CC4(CCCCC4)C3)C[C@@H]1CC(C)C)O2] | +++ | ++ | | |
| 98 | 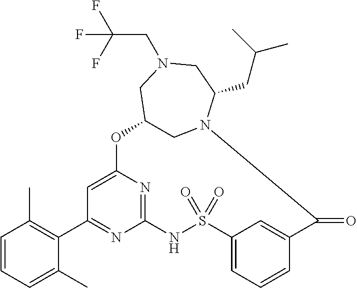 [SMILES: Cc1cccc(C)c1-c1cc2nc(n1)NS(=O)(=O)c1cccc(c1)C(=O)N1C[C@@H](CN(CC(F)(F)F)C[C@@H]1CC(C)C)O2] | ND | + | | |
| 331 | 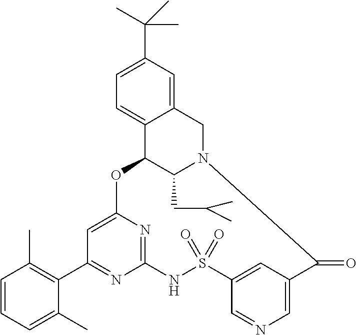 [SMILES: Cc1cccc(C)c1-c1cc2nc(n1)NS(=O)(=O)c1cncc(c1)C(=O)N1Cc3cc(C(C)(C)C)ccc3[C@H](O2)[C@H]1CC(C)C] | +++ | +++ | +++ | +++ |
| 13 | 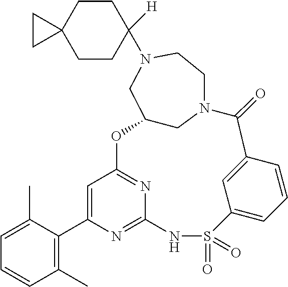 [SMILES: Cc1cccc(C)c1-c1cc2nc(n1)NS(=O)(=O)c1cccc(c1)C(=O)N1CCN(C3CCC4(CC3)CC4)C[C@H](C1)O2] | +++ | +++ | | |
| 12 | 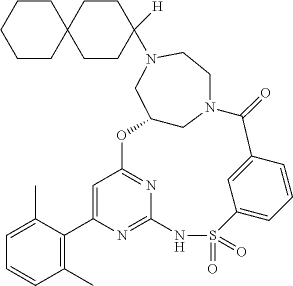 [SMILES: Cc1cccc(C)c1-c1cc2nc(n1)NS(=O)(=O)c1cccc(c1)C(=O)N1CCN(C3CCC4(CCCCC4)CC3)C[C@H](C1)O2] | +++ | +++ | | |
| 41 | 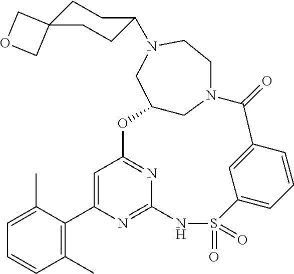 [SMILES: Cc1cccc(C)c1-c1cc2nc(n1)NS(=O)(=O)c1cccc(c1)C(=O)N1CCN(C3CCC4(CC3)COC4)C[C@H](C1)O2] | +++ | +++ | | |
| 116 | 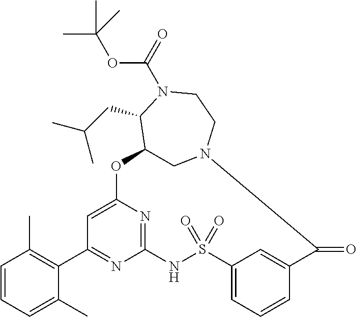 [SMILES: Cc1cccc(C)c1-c1cc2nc(n1)NS(=O)(=O)c1cccc(c1)C(=O)N1CCN(C(=O)OC(C)(C)C)[C@@H](CC(C)C)[C@@H](C1)O2] | +++ | ++ | | |
| 117 | 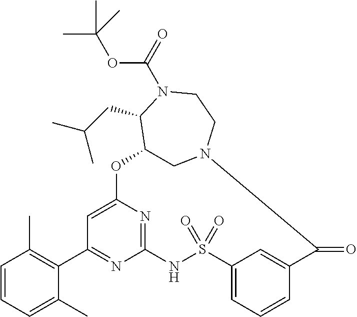 [SMILES: Cc1cccc(C)c1-c1cc2nc(n1)NS(=O)(=O)c1cccc(c1)C(=O)N1CCN(C(=O)OC(C)(C)C)[C@@H](CC(C)C)[C@H](C1)O2] | +++ | +++ | | |
| 71 | 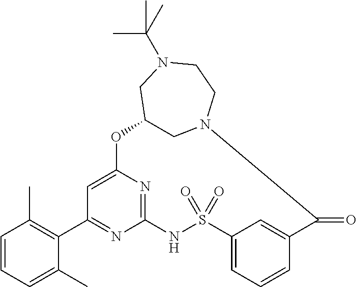 [SMILES: Cc1cccc(C)c1-c1cc2nc(n1)NS(=O)(=O)c1cccc(c1)C(=O)N1CCN(C(C)(C)C)C[C@H](C1)O2] | +++ | +++ | | |
| 38 | 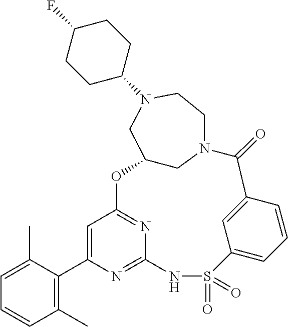 [SMILES: Cc1cccc(C)c1-c1cc2nc(n1)NS(=O)(=O)c1cccc(c1)C(=O)N1CCN([C@H]3CC[C@@H](F)CC3)C[C@H](C1)O2] | +++ | +++ | | |
| 39 | 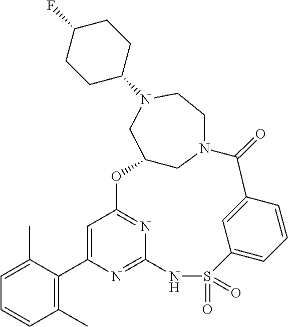 [SMILES: Cc1cccc(C)c1-c1cc2nc(n1)NS(=O)(=O)c1cccc(c1)C(=O)N1CCN([C@H]3CC[C@@H](F)CC3)C[C@H](C1)O2] | ++ | +++ | | |
| 124 | 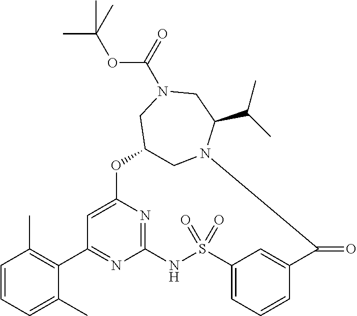 [SMILES: Cc1cccc(C)c1-c1cc2nc(n1)NS(=O)(=O)c1cccc(c1)C(=O)N1C[C@@H](CN(C(=O)OC(C)(C)C)C[C@H]1C(C)C)O2] | +++ | +++ | | |
| 123 | 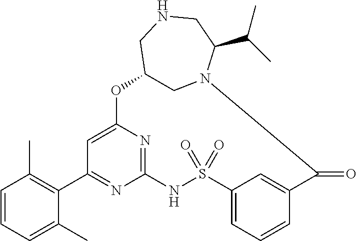 [SMILES: Cc1cccc(C)c1-c1cc2nc(n1)NS(=O)(=O)c1cccc(c1)C(=O)N1C[C@@H](CNC[C@H]1C(C)C)O2] | ++ | ++ | | |
| 125 | 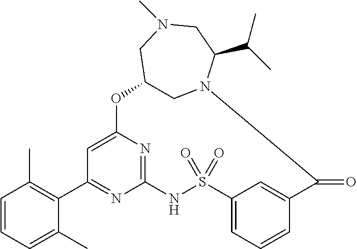 [SMILES: Cc1cccc(C)c1-c1cc2nc(n1)NS(=O)(=O)c1cccc(c1)C(=O)N1C[C@@H](CN(C)C[C@H]1C(C)C)O2] | +++ | +++ | | |
| 103 | 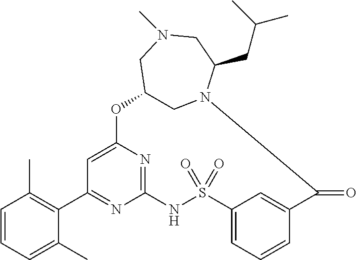 [SMILES: Cc1cccc(C)c1-c1cc2nc(n1)NS(=O)(=O)c1cccc(c1)C(=O)N1C[C@@H](CN(C)C[C@H]1CC(C)C)O2] | +++ | +++ | | |
| 99 | 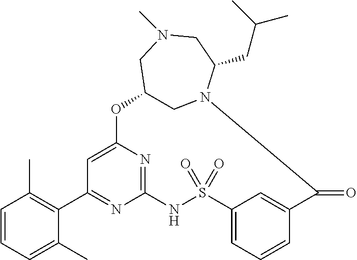 [SMILES: Cc1cccc(C)c1-c1cc2nc(n1)NS(=O)(=O)c1cccc(c1)C(=O)N1C[C@@H](CN(C)C[C@@H]1CC(C)C)O2] | +++ | ++ | | |
| 129 | 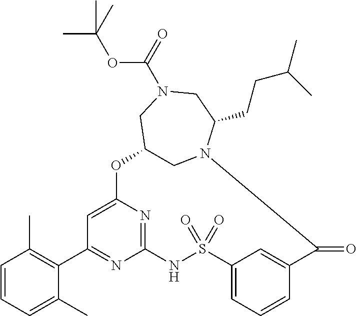 [SMILES: Cc1cccc(C)c1-c1cc2nc(n1)NS(=O)(=O)c1cccc(c1)C(=O)N1C[C@@H](CN(C(=O)OC(C)(C)C)C[C@@H]1CCC(C)C)O2] | +++ | ++ | | |
| 130 | 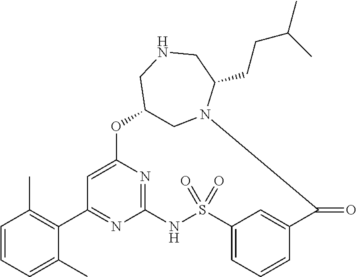 [SMILES: Cc1cccc(C)c1-c1cc2nc(n1)NS(=O)(=O)c1cccc(c1)C(=O)N1C[C@@H](CNC[C@@H]1CCC(C)C)O2] | +++ | +++ | | |
| 126 | 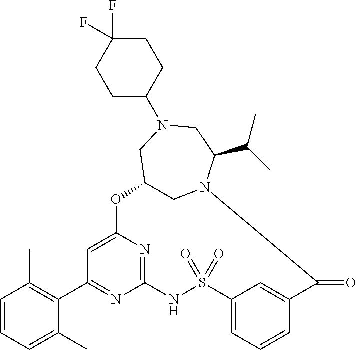 [SMILES: Cc1cccc(C)c1-c1cc2nc(n1)NS(=O)(=O)c1cccc(c1)C(=O)N1C[C@@H](CN(C3CCC(F)(F)CC3)C[C@H]1C(C)C)O2] | +++ | +++ | | |
| 127 | 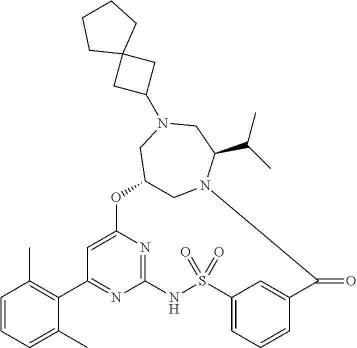 [SMILES: Cc1cccc(C)c1-c1cc2nc(n1)NS(=O)(=O)c1cccc(c1)C(=O)N1C[C@@H](CN(C3CC4(CCCC4)C3)C[C@H]1C(C)C)O2] | +++ | ++ | | |
| 128 | 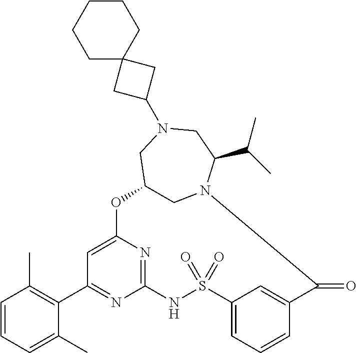 [SMILES: Cc1cccc(C)c1-c1cc2nc(n1)NS(=O)(=O)c1cccc(c1)C(=O)N1C[C@@H](CN(C3CC4(CCCCC4)C3)C[C@H]1C(C)C)O2] | +++ | ++ | | |
| 131 | 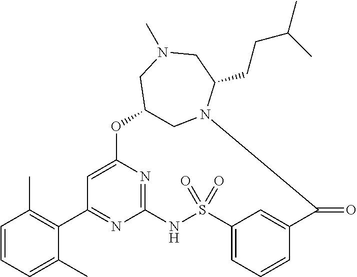 [SMILES: Cc1cccc(C)c1-c1cc2nc(n1)NS(=O)(=O)c1cccc(c1)C(=O)N1C[C@@H](CN(C)C[C@@H]1CCC(C)C)O2] | +++ | ++ | | |
| 132 | 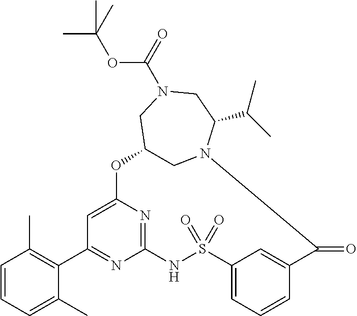 [SMILES: Cc1cccc(C)c1-c1cc2nc(n1)NS(=O)(=O)c1cccc(c1)C(=O)N1C[C@@H](CN(C(=O)OC(C)(C)C)C[C@@H]1C(C)C)O2] | +++ | ++ | | |
| 133 | 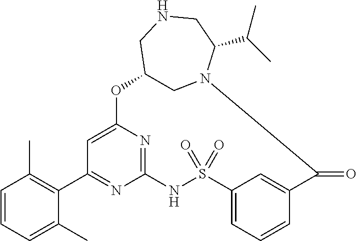 [SMILES: Cc1cccc(C)c1-c1cc2nc(n1)NS(=O)(=O)c1cccc(c1)C(=O)N1C[C@@H](CNC[C@@H]1C(C)C)O2] | +++ | +++ | | |
| 149 | 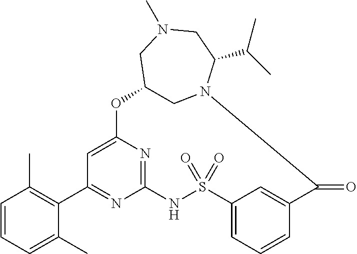 [SMILES: Cc1cccc(C)c1-c1cc2nc(n1)NS(=O)(=O)c1cccc(c1)C(=O)N1C[C@@H](CN(C)C[C@@H]1C(C)C)O2] | +++ | +++ | | |
| 146 | 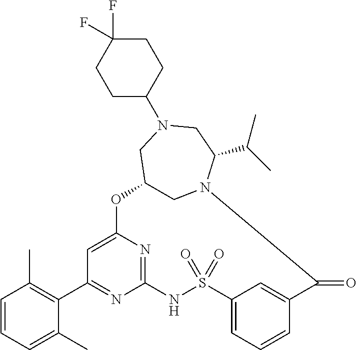 [SMILES: Cc1cccc(C)c1-c1cc2nc(n1)NS(=O)(=O)c1cccc(c1)C(=O)N1C[C@@H](CN(C3CCC(F)(F)CC3)C[C@@H]1C(C)C)O2] | +++ | +++ | | |
| 147 | 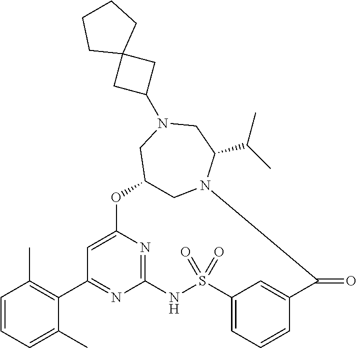 [SMILES: Cc1cccc(C)c1-c1cc2nc(n1)NS(=O)(=O)c1cccc(c1)C(=O)N1C[C@@H](CN(C3CC4(CCCC4)C3)C[C@@H]1C(C)C)O2] | +++ | +++ | | |
| 148 | 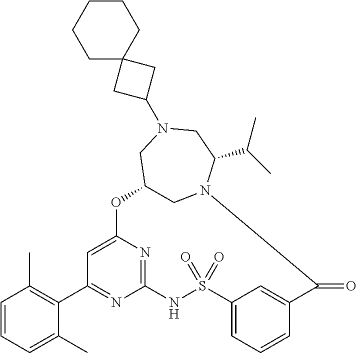 [SMILES: Cc1cccc(C)c1-c1cc2nc(n1)NS(=O)(=O)c1cccc(c1)C(=O)N1C[C@@H](CN(C3CC4(CCCCC4)C3)C[C@@H]1C(C)C)O2] | +++ | +++ | | |
| 134 | 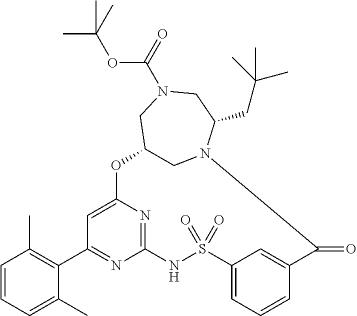 [SMILES: Cc1cccc(C)c1-c1cc2nc(n1)NS(=O)(=O)c1cccc(c1)C(=O)N1C[C@@H](CN(C(=O)OC(C)(C)C)C[C@@H]1CC(C)(C)C)O2] | +++ | ++ | | |
| 135 | 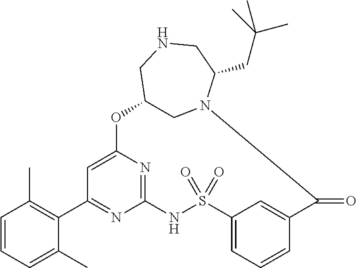 [SMILES: Cc1cccc(C)c1-c1cc2nc(n1)NS(=O)(=O)c1cccc(c1)C(=O)N1C[C@@H](CNC[C@@H]1CC(C)(C)C)O2] | +++ | ++ | | |
| 136 | 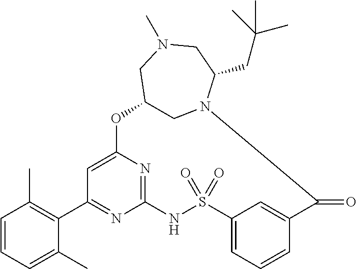 [SMILES: Cc1cccc(C)c1-c1cc2nc(n1)NS(=O)(=O)c1cccc(c1)C(=O)N1C[C@@H](CN(C)C[C@@H]1CC(C)(C)C)O2] | +++ | +++ | | |
| 238 | 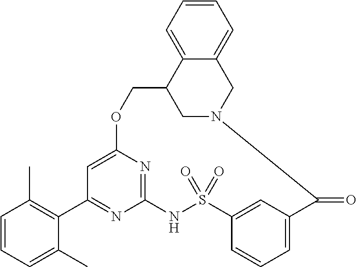 [SMILES: Cc1cccc(C)c1-c1cc2nc(n1)NS(=O)(=O)c1cccc(c1)C(=O)N1Cc3ccccc3C(CO2)C1] | | | +++ | +++ |
| 118 | 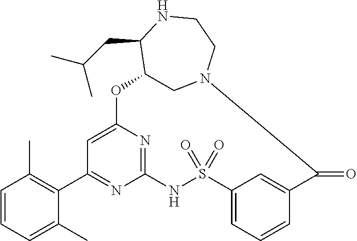 [SMILES: Cc1cccc(C)c1-c1cc2nc(n1)NS(=O)(=O)c1cccc(c1)C(=O)N1CCN[C@H](CC(C)C)[C@H](C1)O2] | | | +++ | +++ |
| 119 | 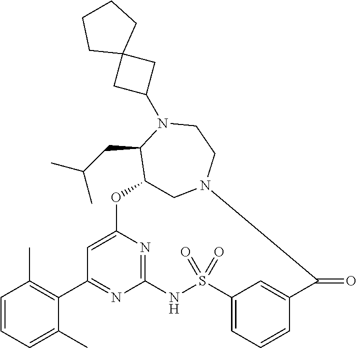 [SMILES: Cc1cccc(C)c1-c1cc2nc(n1)NS(=O)(=O)c1cccc(c1)C(=O)N1CCN(C3CC4(CCCC4)C3)[C@H](CC(C)C)[C@H](C1)O2] | | | +++ | +++ |
| 120 | 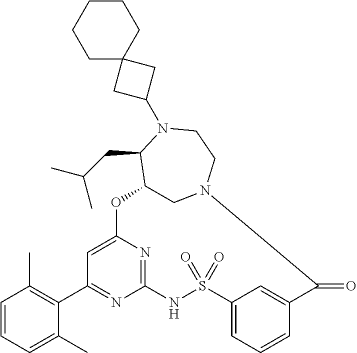 [SMILES: Cc1cccc(C)c1-c1cc2nc(n1)NS(=O)(=O)c1cccc(c1)C(=O)N1CCN(C3CC4(CCCCC4)C3)[C@H](CC(C)C)[C@H](C1)O2] | | | +++ | +++ |
| 121 | 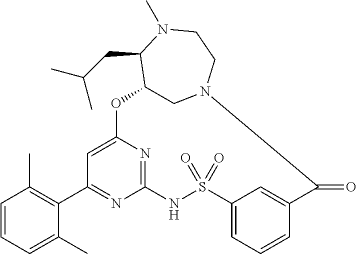 [SMILES: Cc1cccc(C)c1-c1cc2nc(n1)NS(=O)(=O)c1cccc(c1)C(=O)N1CCN(C)[C@H](CC(C)C)[C@H](C1)O2] | | | +++ | +++ |
| 122 | 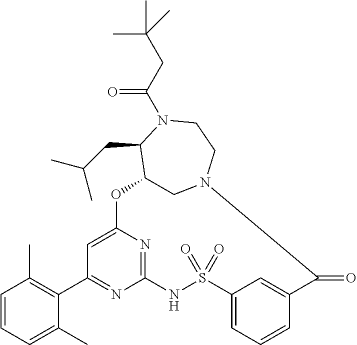 [SMILES: Cc1cccc(C)c1-c1cc2nc(n1)NS(=O)(=O)c1cccc(c1)C(=O)N1CCN(C(=O)CC(C)(C)C)[C@H](CC(C)C)[C@H](C1)O2] | | | +++ | +++ |
| 190 | 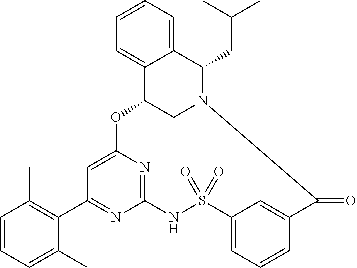 [SMILES: Cc1cccc(C)c1-c1cc2nc(n1)NS(=O)(=O)c1cccc(c1)C(=O)N1C[C@H](O2)c2ccccc2[C@@H]1CC(C)C] | | | +++ | ++ |
| 191 | 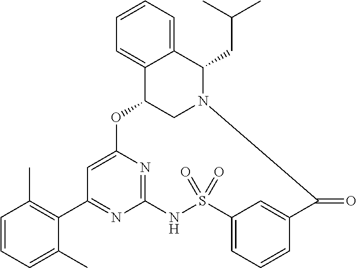 [SMILES: Cc1cccc(C)c1-c1cc2nc(n1)NS(=O)(=O)c1cccc(c1)C(=O)N1C[C@H](O2)c2ccccc2[C@@H]1CC(C)C] | | | +++ | ++ |
| 218 | 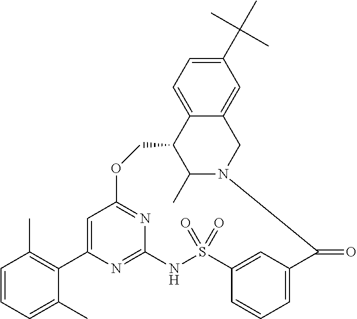 [SMILES: Cc1cccc(C)c1-c1cc2nc(n1)NS(=O)(=O)c1cccc(c1)C(=O)N1Cc3cc(C(C)(C)C)ccc3[C@@H](CO2)C1C] | | | ND | + |
| 219 | 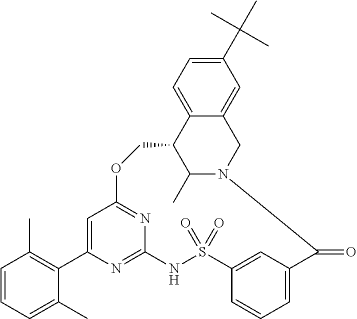 [SMILES: Cc1cccc(C)c1-c1cc2nc(n1)NS(=O)(=O)c1cccc(c1)C(=O)N1Cc3cc(C(C)(C)C)ccc3[C@@H](CO2)C1C] | | | +++ | +++ |
| 330 | 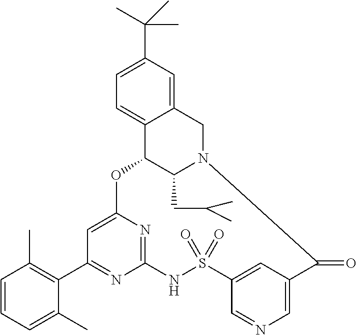 [SMILES: Cc1cccc(C)c1-c1cc2nc(n1)NS(=O)(=O)c1cncc(c1)C(=O)N1Cc3cc(C(C)(C)C)ccc3[C@@H](O2)[C@H]1CC(C)C] | | | | |
| 137 | 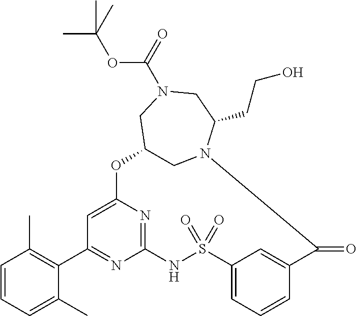 [SMILES: Cc1cccc(C)c1-c1cc2nc(n1)NS(=O)(=O)c1cccc(c1)C(=O)N1C[C@@H](CN(C(=O)OC(C)(C)C)C[C@@H]1CCO)O2] | | | ++ | +++ |
| 181 | 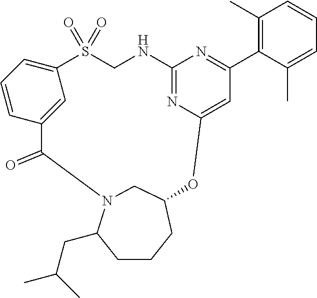 [SMILES: Cc1cccc(C)c1-c1cc2nc(n1)NCS(=O)(=O)c1cccc(c1)C(=O)N1C[C@@H](CCCC1CC(C)C)O2] | | | +++ | ++ |
| 180 | 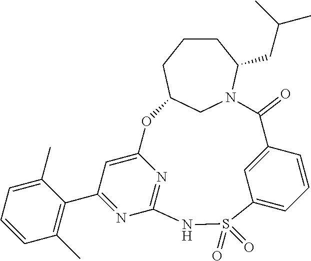 [SMILES: Cc1cccc(C)c1-c1cc2nc(n1)NS(=O)(=O)c1cccc(c1)C(=O)N1C[C@@H](CCC[C@@H]1CC(C)C)O2] | | | +++ | ++ |
| 179 | 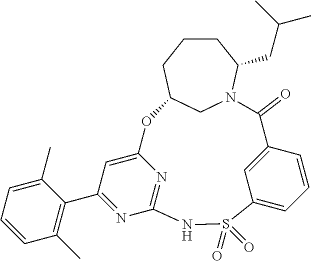 [SMILES: Cc1cccc(C)c1-c1cc2nc(n1)NS(=O)(=O)c1cccc(c1)C(=O)N1C[C@@H](CCC[C@@H]1CC(C)C)O2] | | | ND | + |
| 7 | 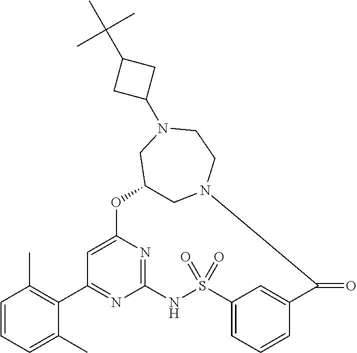 [SMILES: Cc1cccc(C)c1-c1cc2nc(n1)NS(=O)(=O)c1cccc(c1)C(=O)N1CCN(C3CC(C(C)(C)C)C3)C[C@H](C1)O2] | | | | |
| 237 | 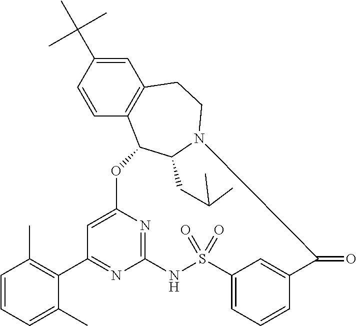 [SMILES: Cc1cccc(C)c1-c1cc2nc(n1)NS(=O)(=O)c1cccc(c1)C(=O)N1CCc3cc(C(C)(C)C)ccc3[C@@H](O2)[C@H]1CC(C)C] | | | +++ | +++ |
| 329 | 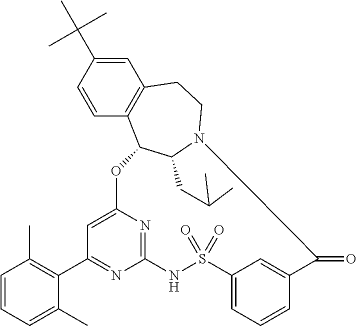 [SMILES: Cc1cccc(C)c1-c1cc2nc(n1)NS(=O)(=O)c1cccc(c1)C(=O)N1CCc3cc(C(C)(C)C)ccc3[C@@H](O2)[C@H]1CC(C)C] | | | +++ | +++ |
| 236 | 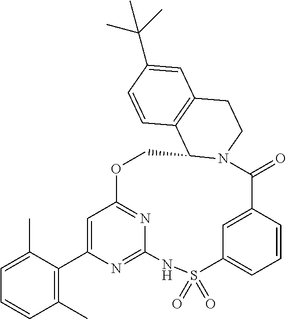 [SMILES: Cc1cccc(C)c1-c1cc2nc(n1)NS(=O)(=O)c1cccc(c1)C(=O)N1CCc3cc(C(C)(C)C)ccc3[C@H]1CO2] | | | +++ | +++ |
| 235 | 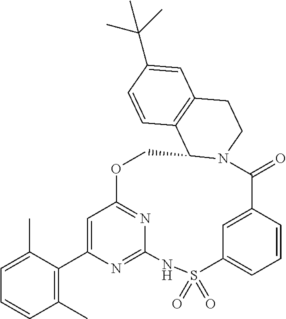 [SMILES: Cc1cccc(C)c1-c1cc2nc(n1)NS(=O)(=O)c1cccc(c1)C(=O)N1CCc3cc(C(C)(C)C)ccc3[C@H]1CO2] | | | +++ | ++ |
| 234 | 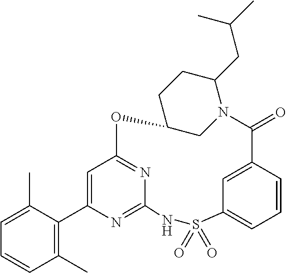 [SMILES: Cc1cccc(C)c1-c1cc2nc(n1)NS(=O)(=O)c1cccc(c1)C(=O)N1C[C@@H](CCC1CC(C)C)O2] | | | +++ | +++ |

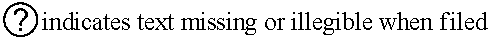

The following tables represents CFTR modulating activity for representative compounds of the disclosure generated using one or more of the assays disclosed herein (EC₅₀: +++ is <1 μM; ++ is 1-<3 μM; + is 3-<30 μM; and ND is “not detected in this assay.” 0 Activity: +++ is >60%; ++ is 30-60%; + is <30%).

TABLE 15

| Cmpd | | HBE | HBE Max |
|---|---|---|---|
| Number | Structure | EC₅₀ (μM) | Activity (%) |
| 328 | 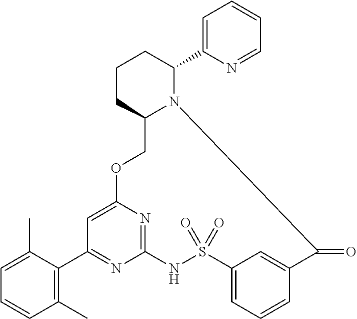 [SMILES: Cc1cccc(C)c1-c1cc2nc(n1)NS(=O)(=O)c1cccc(c1)C(=O)N1[C@H](CCC[C@@H]1c1ccccn1)CO2] | ++ | ++ |
| 327 | 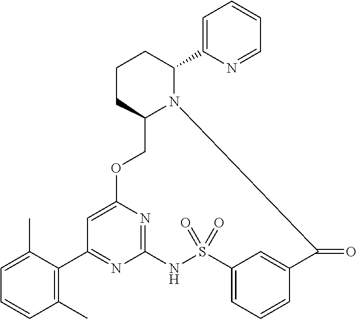 [SMILES: Cc1cccc(C)c1-c1cc2nc(n1)NS(=O)(=O)c1cccc(c1)C(=O)N1[C@H](CCC[C@@H]1c1ccccn1)CO2] | + | ++ |
| 371 | 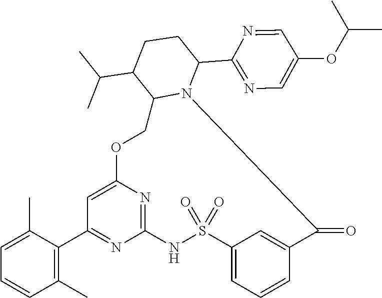 [SMILES: Cc1cccc(C)c1-c1cc2nc(n1)NS(=O)(=O)c1cccc(c1)C(=O)N1C(c3ncc(OC(C)C)cn3)CCC(C(C)C)C1CO2] | +++ | +++ |

### VI. Synthesis of Compounds 386-426

General UPLC/HPLC Analytical Methods

LC method X: Luna C₁₈(2) 50×3 mm, 3 μm. run: 2.5 min. Mobile phase: Initial 95% H₂O 0.1% FA/5% MeCN 0.1% FA, linear grad to 95% MeCN 0.1% FA over 1.3 min, hold 1.2 min 95% CH₃CN 0.1% FA, T: 45° C., Flow: 1.5 mL/min.

LC method Y: Luna C₁₈(2) 50×3 mm, 3 μm. Temperature: 45° C., Flow: 1.5 mL/min, Run time: 3.5 min. Mobile phase conditions: Initial 95% H₂O 0.1% FA/5% MeCN 0.1% FA, linear gradient to 95% MeCN 0.1% FA over 1.3 min then hold for 2.2 minute at 95% CH₃CN 0.1% FA. MSD: ESI Positive.

LCMS method Z: SunFire C₁₈ 4.6×75 mm 5 μM, 6 min run, 50-95% ACN/Water (0.1% FA modifier), 1.5 min equilibration, gradient over 3 min, hold 3 min. 1.5 mL/min.

LCMS method 1A: C₁₈ SunFire 4.6×75 mm 5 m, 10 min run, 50-95% ACN/Water (0.1% FA modifier), 1.5 min equilibration, gradient over 3 min, hold 7 min. 1.5 mL/min.

LC method 1B: XBridge C₁₈ 4.6×75 mm 5 μm, Initial Gradient at 95% NH₄HCO₃/5% MeCN 6 min run with 1 min equilibration Gradient 0 to 3 min at 95% MeCN and hold for 3 minutes. Flow 1.5 mL/min.

### Example 143: Preparation of Compound 386

### Step 1: 3,3-Dimethylpiperidine-2-carboxylic acid

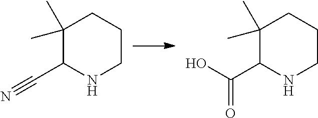

In a round bottom flask 3,3-dimethylpiperidine-2-carbonitrile (8.51 g, 49.443 mmol) and HCl (in water) (83 mL of 12 M, 996.00 mmol) were introduced. The mixture was then refluxed for 3 days. The crude was evaporated to dryness. The residue was solubilized in MeOH (150 mL) then Lewatit® MP-62 free base was added until pH=8. The mixture was then filtered, and the filtrate was evaporated under reduced pressure. The residue was triturated in CH₂Cl₂ giving the desired 3,3-dimethylpiperidine-2-carboxylic acid (7.895 g, 101%) to precipitate as an off-white solid. ¹H NMR (400 MHz, METHANOL-d₄) δ 3.37-3.21 (m, 2H, overlap with solvent), 3.02-2.80 (m, 1H), 1.91-1.47 (m, 4H), 1.27-1.17 (m, 3H), 1.07-0.99 (m, 3H), (2H missing, labile protons). ESI-MS m/z calc. 157.1103, found 156.2 (M−1); Retention time: 0.41 minutes. LC method X.

### Step 2: 1-tert-Butoxycarbonyl-3,3-dimethyl-piperidine-2-carboxylic acid

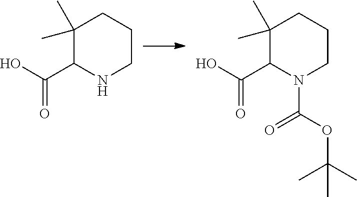 [SMILES: CC1(C)CCCNC1C(=O)O ; CC(C)(C)OC(=O)N1CCCC(C)(C)C1C(=O)O]

3,3-Dimethylpiperidine-2-carboxylic acid (11.16 g, 64.599 mmol) was dissolved in water (100 mL) with sodium carbonate (30 g, 283.05 mmol) then tert-butoxycarbonyl tert-butyl carbonate (19.000 g, 20 mL, 87.057 mmol) was added dropwise dissolved in dioxane (150 mL) and the reaction mixture was stirred at room temperature overnight. More tert-butoxycarbonyl tert-butyl carbonate (19.000 g, 20 mL, 87.057 mmol) and sodium carbonate (17 g, 160.40 mmol) were added, and the reaction was stirred 3 days at 25-30° C. The reaction mixture was diluted with water until all solid were dissolved and the mixture was stirred at room temperature for 1 h. The resulting solution was washed heptanes (200 mL) to remove underacted excess BOC₂O. Ethyl acetate (100 mL) was added to the aqueous phase which was acidified at 0° C. until pH=3-4 with 6 N aqueous HCl. The aqueous phase was separated and washed with ethyl acetate (2×200 mL). The organic phases were combined, dried over anhydrous magnesium sulfate, filtered and concentrated under reduced pressure to provide crude 1-tert-butoxycarbonyl-3,3-dimethyl-piperidine-2-carboxylic acid (14.7 g, 78%) as a clear yellow oil. ¹H NMR (400 MHz, CDCl₃) δ 4.52-4.24 (m, 1H), 4.10-3.88 (m, 1H), 3.26 (br. s., 1H), 1.76-1.64 (m, 2H), 1.50-1.43 (m, 10H), 1.34-1.28 (m, 1H), 1.07 (s, 6H), (exchangeable proton COOH is missing). ESI-MS m/z calc. 257.1627, found 202.2 (M−55)⁺; Retention time: 1.76 minutes; LC method X.

### Step 3: tert-Butyl 2-(hydroxymethyl)-3,3-dimethyl-piperidine-1-carboxylate

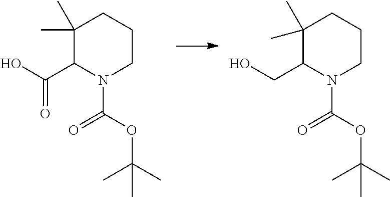 [SMILES: CC(C)(C)OC(=O)N1CCCC(C)(C)C1C(=O)O ; CC(C)(C)OC(=O)N1CCCC(C)(C)C1CO]

1-tert-Butoxycarbonyl-3,3-dimethyl-piperidine-2-carboxylic acid (7.83 g, 22.426 mmol) was dissolved in THE (80 mL) and cooled to 0° C. Borane-THF complex (42.7 mL of 1 M, 42.700 mmol) was then added dropwise at 0° C. and the resultant solution was stirred for 1 h. The reaction mixture was then warmed to 25° C. and stirred overnight. Upon completion, the reaction contents were cooled to 0° C., saturated aqueous sodium bicarbonate (5 mL) was added slowly to quench excess reagent, and water (10 mL) was added to dissolve the precipitated salts. The crude reaction contents were then extracted with CH₂Cl₂ (4×10 mL), and the combined organic extracts were washed with saturated aqueous sodium bicarbonate (3×20 mL) and water (3×20 mL), dried (Na₂SO₄), filtered, and concentrated to give tert-butyl 2-(hydroxymethyl)-3,3-dimethyl-piperidine-1-carboxylate (7 g, 128%) as a colorless oil. ESI-MS m/z calc. 243.18344, found null (M+)⁺; 188.2 (M−55)⁺; Retention time: 1.74 minutes; LC method X.

### Step 4: tert-Butyl 2-[[tert-butyl(diphenyl)silyl]oxymethyl]-3,3-dimethyl-piperidine-1-carboxylate

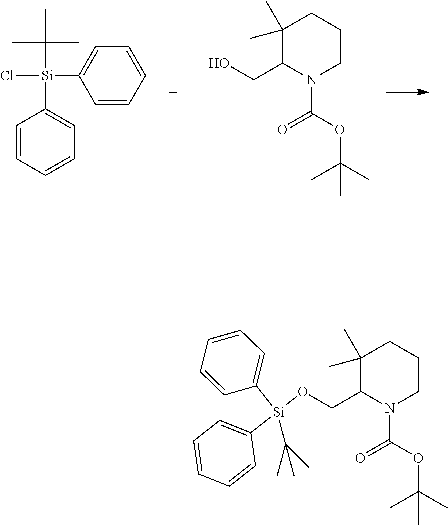 [SMILES: CC(C)(C)[Si](Cl)(c1ccccc1)c1ccccc1 ; CC(C)(C)OC(=O)N1CCCC(C)(C)C1CO ; CC(C)(C)OC(=O)N1CCCC(C)(C)C1CO[Si](c1ccccc1)(c1ccccc1)C(C)(C)C]

Crude tert-butyl 2-(hydroxymethyl)-3,3-dimethyl-piperidine-1-carboxylate (7 g, 28.766 mmol) was dissolved in dry DCM (147 mL) and treated with imidazole (4.12 g, 60.519 mmol), tert-butyl-chloro-diphenyl-silane (11.891 g, 11.25 mL, 43.262 mmol) and 4-Dimethylaminopyridine (175 mg, 1.4325 mmol) at 0° C. The mixture was stirred at 0° C. for 20 min then overnight at room temperature, diluted with DCM, washed with water, dried over Na₂SO₄, and solvent was evaporated under reduced pressure. The resulting brown oil was filtered through a pad of silica gel (Hexane/EtOAc 8:2). The filtrate was evaporated and purified by reverse phase chromatography on a 80 g C₁₈ Gold cartridge using a gradient of MeCN (65% for 3.5 CV then to 100% over 10 CV then 100% for 15 CV) in acidic water (formic acid=0.1% w/w) to give the pure tert-butyl 2-[[tert-butyl(diphenyl)silyl]oxymethyl]-3,3-dimethyl-piperidine-1-carboxylate as a colorless tacky oil. ESI-MS m/z calc. 481.3012, found 382.2 (M−99)⁺; Retention time: 2.79 minutes; LC method Y.

### Step 5: tert-Butyl 2-[[tert-butyl(diphenyl)silyl]oxymethyl]-3,3-dimethyl-6-oxo-piperidine-1-carboxylate

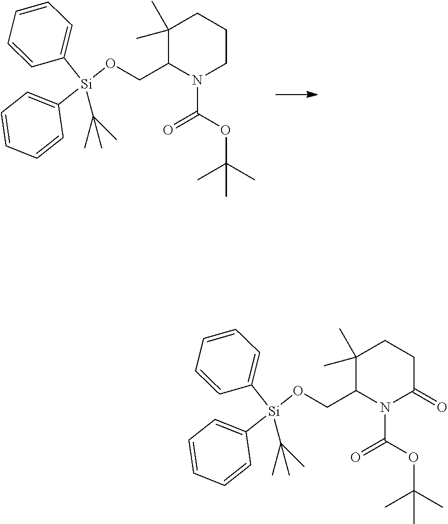 [SMILES: CC(C)(C)OC(=O)N1CCCC(C)(C)C1CO[Si](c1ccccc1)(c1ccccc1)C(C)(C)C ; CC(C)(C)OC(=O)N1C(=O)CCC(C)(C)C1CO[Si](c1ccccc1)(c1ccccc1)C(C)(C)C]

In 2 L flask equipped with a mechanical stirrer and an internal temperature sensor, was introduced sodium periodate (56 g, 261.81 mmol) and water (565 mL) followed by ruthenium(IV) oxide hydrate (1.2 g, 7.9426 mmol). The mixture was stirred for 10 min. Then a solution of tert-butyl 2-[[tert-butyl(diphenyl)silyl]oxymethyl]-3,3-dimethyl-piperidine-1-carboxylate (25.4 g, 48.982 mmol) in EtOAc (255 mL) was added to the mixture and stirred vigorously. After 1 hour reaction time the phases were separated, the aqueous phase was washed with EtOAc (3×300 mL). To the combined organic phases was added iPr-OH (10 mL) and the mixture was stirred for 45 min then filtered over a pad of Celite. The filtrate was dried over sodium sulfate, filtered then evaporated. The crude was then purified by dry column vacuum chromatography on silica gel using 5, 10 and 15% EtOAc in heptanes to provide tert-butyl 2-[[tert-butyl(diphenyl)silyl]oxymethyl]-3,3-dimethyl-6-oxo-piperidine-1-carboxylate (13.9 g, 57%) as a clear yellow oil. ¹H NMR (400 MHz, CDCl₃) δ 7.71-7.61 (m, 4H), 7.45-7.36 (m, 6H), 3.90-3.84 (m, 1H), 3.77 (dd, J=11.0, 2.0 Hz, 1H), 3.75-3.71 (m, 1H), 2.67-2.51 (m, 2H), 2.37 (td, J=12.0, 7.6 Hz, 1H), 1.49 (s, 10H), 1.13 (s, 3H), 1.05 (s, 3H), 1.03 (s, 9H). ESI-MS m/z calc. 495.2805, found 396.3 (M−99)⁺; Retention time: 4.49 minutes; LC method Z.

### Step 6: tert-Butyl 2-[[tert-butyl(diphenyl)silyl]oxymethyl]-3,3-dimethyl-6-(trifluoromethylsulfonyloxy)-2,4-dihydropyridine-1-carboxylate

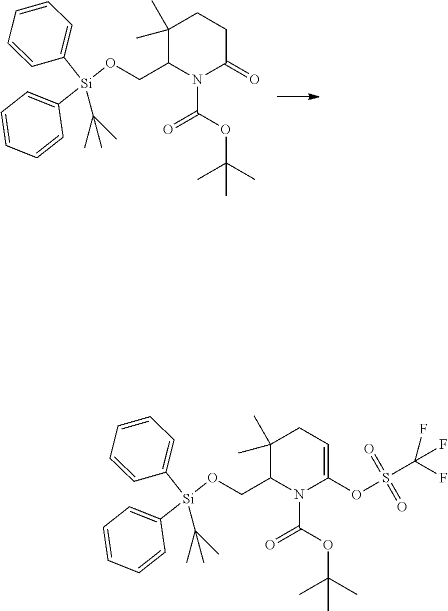 [SMILES: CC(C)(C)OC(=O)N1C(=O)CCC(C)(C)C1CO[Si](c1ccccc1)(c1ccccc1)C(C)(C)C ; CC(C)(C)OC(=O)N1C(OS(=O)(=O)C(F)(F)F)=CCC(C)(C)C1CO[Si](c1ccccc1)(c1ccccc1)C(C)(C)C]

A solution of tert-butyl 2-[[tert-butyl(diphenyl)silyl]oxymethyl]-3,3-dimethyl-6-oxo-piperidine-1-carboxylate (2.0 g, 3.9820 mmol) in anhydrous THE (44 mL) was cooled at −78° C. under nitrogen. Lithium bis(trimethylsilyl)amide (in THF) (7 mL of 1 M, 7.0000 mmol) was added dropwise over 10 min at −78° C. (dry ice/acetone). The reaction was stirred at the same temperature for 0.5 hour. A solution of N-(5-chloro-2-pyridyl)-1,1,1-trifluoro-N-(trifluoromethylsulfonyl)methanesulfonamide (2.22 g, 5.6534 mmol) in anhydrous THE (4.4 mL) was added to the reaction mixture dropwise over 10 min. The reaction was then stirred at −78° C. for 1 hour. The reaction mixture was then warmed up to −40° C. (dry ice+MeOH/H₂O (1:1)) and stirred for 15 min. The reaction was quenched with saturated sodium bicarbonate (15 mL) at −40° C. and then slowly raised to rt. Water (100 mL) was added to the mixture and the aqueous phase was extracted with MTBE (3×150 mL). The combined organic layers were washed with brine (2×50 mL) mL), dried over anhydrous Na₂SO₄, filtered and concentrated under vacuum. The crude product was purified by reverse phase chromatography on a 15.5 g Cis Gold cartridge using a gradient of MeCN (75% for 3.5 CV then to 100% over 10 CV then 100% for 10 CV) in acidic water (formic acid=0.1% w/w). The collected fractions containing the product were combined, evaporated to dryness under reduced pressure and filtered through a pad of silica gel (40 g, n-Hex/EtOAc 95:5) to provide the pure tert-butyl 2-[[tert-butyl(diphenyl)silyl]oxymethyl]-3,3-dimethyl-6-(trifluoromethylsulfonyloxy)-2,4-dihydropyridine-1-carboxylate (2.47 g, 99%) as white solid. ¹H NMR (400 MHz, CDCl₃) δ 7.76-7.66 (m, 4H), 7.46-7.35 (m, 6H), 5.12 (t, J=3.8 Hz, 1H), 4.25-4.12 (m, 1H), 3.75-3.66 (m, 1H), 3.64-3.58 (m, 1H), 1.87 (d, J=3.7 Hz, 2H), 1.53 (s, 9H), 1.04 (s, 9H), 0.92 (s, 3H), 0.82 (s, 3H). 19F NMR (377 MHz, CDCl₃) δ −74.28 (s, 3F). ESI-MS m/z calc. 627.2298, found 650.2 (M+23)⁺; Retention time: 5.3 minutes; LC method Z.

### Step 7: tert-Butyl 2-[[tert-butyl(diphenyl)silyl]oxymethyl]-3,3-dimethyl-6-(4,4,5,5-tetramethyl-1,3,2-dioxaborolan-2-yl)-2,4-dihydropyridine-1-carboxylate

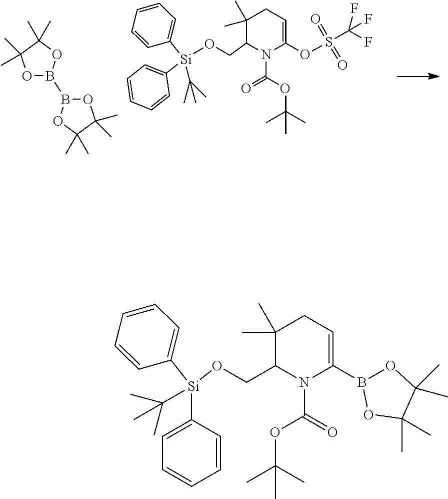

A 100 mL Schlenk tube was charged with tert-butyl 2-[[tert-butyl(diphenyl)silyl]oxymethyl]-3,3-dimethyl-6-(trifluoromethylsulfonyloxy)-2,4-dihydropyridine-1-carboxylate (3 g, 4.7739 mmol), 4,4,5,5-tetramethyl-2-(4,4,5,5-tetramethyl-1,3,2-dioxaborolan-2-yl)-1,3,2-dioxaborolane (1.94 g, 7.6396 mmol), finely ground potassium carbonate (1.72 g, 12.445 mmol), triphenylphosphine (252 mg, 0.9608 mmol), bis(triphenylphosphine)palladium(II)chloride (336 mg, 0.4787 mmol) under nitrogen atmosphere. A pre-degassed and dry 1,4-dioxane (48 mL) was added to the reaction flask. The mixture was sonicated for 20 seconds then heated at 85° C. overnight. The reaction was cooled to room temperature and the crude was filtered over a pad of celite. The filtrate was then evaporated. The residue was subsequently passed through a pad of silica gel (n-Hex/EtOAc 95:5) to give the pure tert-butyl 2-[[tert-butyl(diphenyl)silyl]oxymethyl]-3,3-dimethyl-6-(4,4,5,5-tetramethyl-1,3,2-dioxaborolan-2-yl)-2,4-dihydropyridine-1-carboxylate (2.8 g, 94%) as a colorless tacky oil. ESI-MS m/z calc. 605.3708, found 506.3 (M−99)⁺; Retention time: 6.38 minutes; LC method 1A.

### Step 8: 2-Bromo-5-isopropoxy-pyrimidine

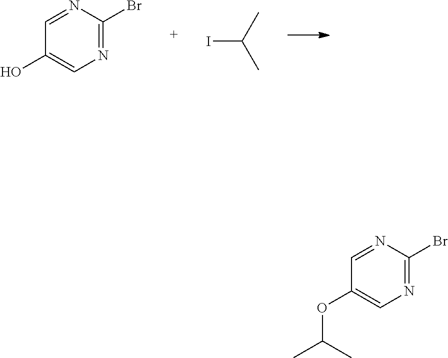

2-Bromopyrimidin-5-ol (3.56 g, 20.34 mmol) and potassium carbonate (5.6 g, 40.52 mmol) were combined in a flask followed by the addition of DMF (35 mL) and then 2-iodopropane (3 mL, 30.05 mmol). The reaction mixture was heated at 50° C. for 1 hour under a nitrogen atmosphere. The reaction mixture was cooled to room temperature and filtered. The filtrate was diluted with ethyl acetate and saturated aqueous sodium chloride solution was added. The layers were separated, and the organic layer was washed four times with saturated aqueous sodium chloride solution. The organic layer was dried over sodium sulfate, filtered, and concentrated to a yellow oil under reduced pressure. The crude material was purified by silica gel column chromatography using a gradient of 0-20% ethyl acetate in hexanes to provide 2-bromo-5-isopropoxy-pyrimidine (4.14 g, 94%) as a white solid. ¹H NMR (400 MHz, DMSO-d₆) δ 8.48 (s, 2H), 4.79 (p, J=6.0 Hz, 1H), 1.30 (d, J=6.0 Hz, 6H). ESI-MS m/z calc. 215.98982, found 216.97 (M+1)⁺; Retention time: 0.47 minutes; LC method D.

### Step 9: tert-Butyl 2-[[tert-butyl(diphenyl)silyl]oxymethyl]-6-(5-isopropoxypyrimidin-2-yl)-3,3-dimethyl-2,4-dihydropyridine-1-carboxylate

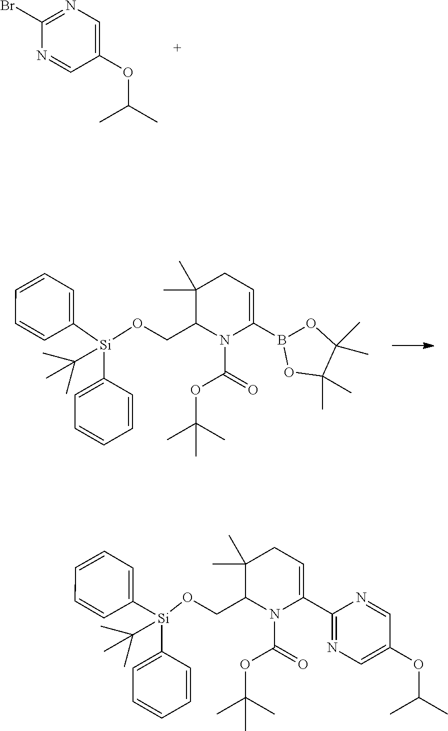 [SMILES: CC(C)Oc1cnc(Br)nc1 ; CC(C)(C)OC(=O)N1C(B2OC(C)(C)C(C)(C)O2)=CCC(C)(C)C1CO[Si](c1ccccc1)(c1ccccc1)C(C)(C)C ; CC(C)Oc1cnc(C2=CCC(C)(C)C(CO[Si](c3ccccc3)(c3ccccc3)C(C)(C)C)N2C(=O)OC(C)(C)C)nc1]

A 20 mL tube was charged with tert-butyl 2-[[tert-butyl(diphenyl)silyl]oxymethyl]-3,3-dimethyl-6-(4,4,5,5-tetramethyl-1,3,2-dioxaborolan-2-yl)-2,4-dihydropyridine-1-carboxylate (500 mg, 0.8247 mmol), 2-bromo-5-isopropoxy-pyrimidine (269 mg, 1.2393 mmol) and cesium hydroxide hydrate (277 mg, 1.6495 mmol) followed by dry toluene (5.5000 mL) and dry 1,4-dioxane (5.5000 mL). The mixture was bubbled with N2 for 15 min under stirring. Then tetrakis(triphenylphosphine)palladium (95.6 mg, 0.0827 mmol) was added and the mixture was bubbled for 5 min. The tube was sealed, and the mixture was heated at 110° C. overnight. The reaction was cooled to room temperature, diluted with EtOAc and filtered over a pad of Celite and silica gel (EtOAc). The filtrate was evaporated and the residue was subsequently purified by flash chromatography on a 40 g Cis Gold cartridge using a gradient of EtOAc in n-hexane (5% for 3.5 CV then to 25% over 20 CV) to provide the pure tert-butyl 2-[[tert-butyl(diphenyl)silyl]oxymethyl]-6-(5-isopropoxypyrimidin-2-yl)-3,3-dimethyl-2,4-dihydropyridine-1-carboxylate (406 mg, 80%) as a colorless tacky oil. ESI-MS m/z calc. 615.3492, found 616.3 (M+1)⁺; Retention time: 5.28 minutes; LC method 1A.

### Step 10: tert-Butyl-[[6-(5-isopropoxypyrimidin-2-yl)-3,3-dimethyl-2,4-dihydro-1H-pyridin-2-yl]methoxy]-diphenyl-silane

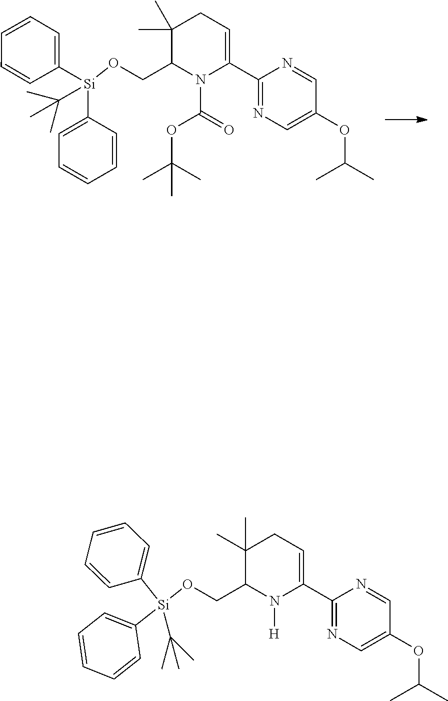 [SMILES: CC(C)Oc1cnc(C2=CCC(C)(C)C(CO[Si](c3ccccc3)(c3ccccc3)C(C)(C)C)N2C(=O)OC(C)(C)C)nc1 ; CC(C)Oc1cnc(C2=CCC(C)(C)C(CO[Si](c3ccccc3)(c3ccccc3)C(C)(C)C)N2)nc1]

In a 25 mL flask was introduced tert-butyl 2-[[tert-butyl(diphenyl)silyl]oxymethyl]-6-(5-isopropoxypyrimidin-2-yl)-3,3-dimethyl-2,4-dihydropyridine-1-carboxylate (838 mg, 1.3593 mmol) and DCM (8.4 mL). Then trifluoroacetic acid (4.6620 g, 3.15 mL, 40.886 mmol) was added dropwise at room temperature. The reaction was stirred overnight at room temperature. The crude was evaporated to dryness to give tert-butyl-[[6-(5-isopropoxypyrimidin-2-yl)-3,3-dimethyl-2,4-dihydro-1H-pyridin-2-yl]methoxy]-diphenyl-silane (trifluoroacetate salt) (1.1 g, 99%) as a yellow tacky oil. ESI-MS m/z calc. 515.2968, found 516.3 (M+1)⁺; Retention time: 1.84 minutes. LC method X.

### Step 11: tert-Butyl-[[6-(5-isopropoxypyrimidin-2-yl)-3,3-dimethyl-2-piperidyl]methoxy]-diphenyl-silane

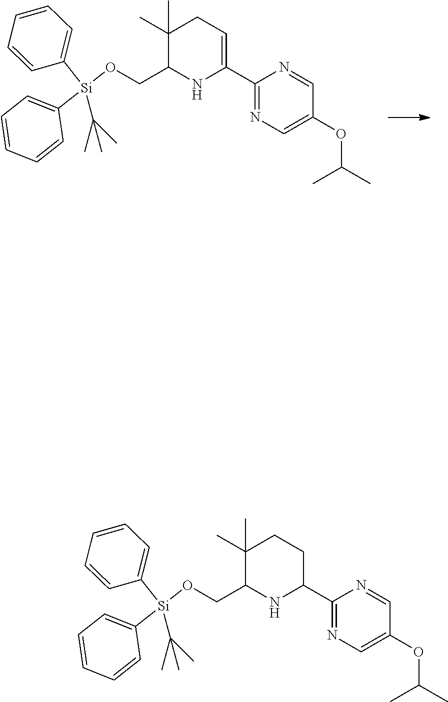 [SMILES: CC(C)Oc1cnc(C2=CCC(C)(C)C(CO[Si](c3ccccc3)(c3ccccc3)C(C)(C)C)N2)nc1 ; CC(C)Oc1cnc(C2CCC(C)(C)C(CO[Si](c3ccccc3)(c3ccccc3)C(C)(C)C)N2)nc1]

In a 25 mL round bottom flask was tert-butyl-[[6-(5-isopropoxypyrimidin-2-yl)-3,3-dimethyl-2,4-dihydro-1H-pyridin-2-yl]methoxy]-diphenyl-silane (trifluoroacetate salt) (415 mg, 0.6590 mmol) solubilized in THE (10.5 mL) then triethylamine (163.35 mg, 0.225 mL, 1.6143 mmol) was added to the mixture. After 5 min stirring sodium triacetoxyborohydride (682 mg, 3.2179 mmol) was added to the mixture at room temperature and the mixture was stirred overnight. The reaction was quench by addition of H₂O (7.5 mL) and AcOH (2 mL). The crude mixture was then washed with EtOAC (3×10 mL). The combined organic phases were dried over Na₂SO₄, filtered, and concentrated under reduced pressure. The crude was purified by reverse phase chromatography on a 30 g Cis Gold cartridge using a gradient of MeCN (40% for 3.5 CV then to 100% over 13CV then 100% for 15 CV) in basic water (NH₄OH buffer, pH=10) to give the pure tert-butyl-[[6-(5-isopropoxypyrimidin-2-yl)-3,3-dimethyl-2-piperidyl]methoxy]-diphenyl-silane (211 mg, 59%). ESI-MS m/z calc. 517.31244, found 518.3 (M+1)⁺; Retention time: 5.24 minutes; LC method 1B.

### Step 12: tert-Butyl-[[6-(5-isopropoxypyrimidin-2-yl)-3,3-dimethyl-2-piperidyl]methoxy]-diphenyl-silane, diastereomer 1 and tert-butyl-[[6-(5-isopropoxypyrimidin-2-yl)-3,3-dimethyl-2-piperidyl]methoxy]-diphenyl-silane, diastereomer 2

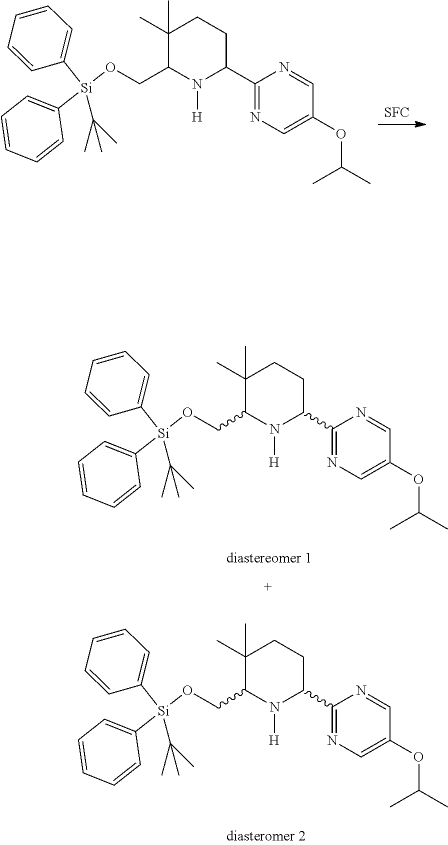

Two different batches of purified tert-butyl-[[6-(5-isopropoxypyrimidin-2-yl)-3,3-dimethyl-2-piperidyl]methoxy]-diphenyl-silane (70 mg, 0.1313 mmol), tert-butyl-[[6-(5-isopropoxypyrimidin-2-yl)-3,3-dimethyl-2-piperidyl]methoxy]-diphenyl-silane (100 mg, 0.1738 mmol) and tert-butyl-[[6-(5-isopropoxypyrimidin-2-yl)-3,3-dimethyl-2-piperidyl]methoxy]-diphenyl-silane (211 mg, 0.3900 mmol) were taken in MeCN and combined. After evaporation under reduced pressure the new batch was purified by reverse phase chromatography on a 30 g C₁₈ Gold cartridge using a gradient of MeCN (40% for 3.5 CV then to 100% over 13CV then 100% for 15 CV) in basic water (NH₄OH buffer, pH=10) to give the pure tert-butyl-[[6-(5-isopropoxypyrimidin-2-yl)-3,3-dimethyl-2-piperidyl]methoxy]-diphenyl-silane (150 mg, 80%) as a yellow tacky oil. The residue was subsequently purified by SFC (Column Lux 5 m, Cellulose 4, 250×21.2 mm, 39.1 mg/injection, concentration 48.9 mg/mL, Column T=40° C., Flow rate 75 mL/min, 25% MeOH) to give tert-butyl-[[6-(5-isopropoxypyrimidin-2-yl)-3,3-dimethyl-2-piperidyl]methoxy]-diphenyl-silane (150 mg, 80%) (Diastereomer 1) as a glassy tacky oil (ESI-MS m/z calc. 517.3125, found 518.3 (M+1)⁺; Retention time: 5.26 minutes) and tert-butyl-[[6-(5-isopropoxypyrimidin-2-yl)-3,3-dimethyl-2-piperidyl]methoxy]-diphenyl-silane (153 mg, 85%) (Diastereomer 2) as a glassy tacky oil (ESI-MS m/z calc. 517.3125, found 518.3 (M+1)⁺; Retention time: 5.26 minutes; LC method 1B.

### Step 13: [6-(5-Isopropoxypyrimidin-2-yl)-3,3-dimethyl-2-piperidyl]methanol, diastereomer 1

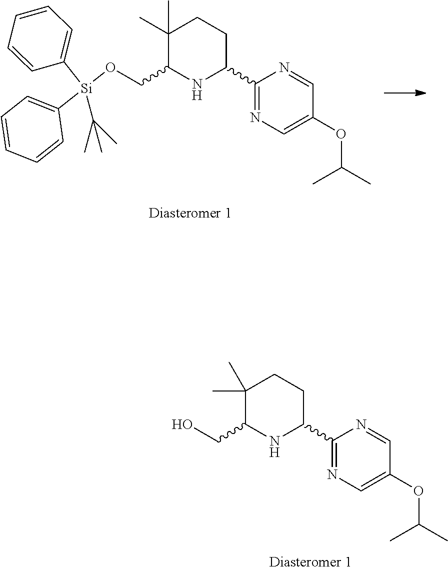 [SMILES: CC(C)Oc1cnc(C2CCC(C)(C)C(CO[Si](c3ccccc3)(c3ccccc3)C(C)(C)C)N2)nc1 ; CC(C)Oc1cnc(C2CCC(C)(C)C(CO)N2)nc1]

In a 50 mL flask was introduced tert-butyl-[[6-(5-isopropoxypyrimidin-2-yl)-3,3-dimethyl-2-piperidyl]methoxy]-diphenyl-silane diastereomer 1 (150 mg, 0.2784 mmol) (SFC peak1) and THE (2.25 mL). The mixture was stirred for 10 min then TBAF (1.11 mL of 1 M, 1.1100 mmol) was added at room temperature. After addition the mixture was warmed up to 40° C. and stirred at this temperature overnight. The reaction was cooled down to room temperature and the volatiles were removed by evaporation under reduced pressure. The crude was purified by reverse-phase chromatography on Cis (column: 30 g HP Gold Cis; gradient: 30 to 100% acetonitrile in water containing 0.1% of formic acid, 15 CV) to afford [6-(5-isopropoxypyrimidin-2-yl)-3,3-dimethyl-2-piperidyl]methanol diastereomer 1 (formate salt) (90 mg, 99%) as a light yellow oil. ESI-MS m/z calc. 279.19467, found 280.2 (M+1)⁺; Retention time: 1.19 minutes; LC method X.

### Step 14: 3-[[4-(2,6-Dimethylphenyl)-6-[[6-(5-isopropoxypyrimidin-2-yl)-3,3-dimethyl-2-piperidyl]methoxy]pyrimidin-2-yl]sulfamoyl]benzoic acid, diastereomer 1

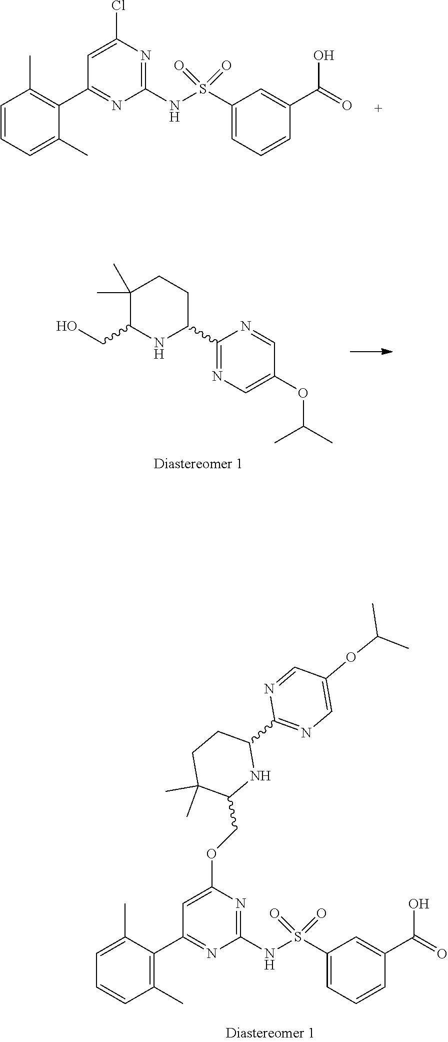 [SMILES: Cc1cccc(C)c1-c1cc(Cl)nc(NS(=O)(=O)c2cccc(C(=O)O)c2)n1 ; CC(C)Oc1cnc(C2CCC(C)(C)C(CO)N2)nc1 ; Cc1cccc(C)c1-c1cc(OCC2NC(c3ncc(OC(C)C)cn3)CCC2(C)C)nc(NS(=O)(=O)c2cccc(C(=O)O)c2)n1]

A solution of [6-(5-isopropoxypyrimidin-2-yl)-3,3-dimethyl-2-piperidyl]methanol diastereomer 1(90 mg, 0.3218 mmol) in anhydrous N,N-dimethylformamide (0.45 mL) was added to a solution of 3-[[4-chloro-6-(2,6-dimethylphenyl)pyrimidin-2-yl]sulfamoyl]benzoic acid (150 mg, 0.3590 mmol) in 2-methyltetrahydrofuran (2.25 mL). The mixture was cooled down to 10-15° C. and then sodium tert-butoxide (200 mg, 2.0811 mmol) was added. The reaction was stirred at 10-15° C. for 2 hours, then stirred overnight at room temperature. The mixture was warmed up to 40° C. for 1 h. The reaction was brought back to rt and sodium tert-butoxide (78 mg, 0.8116 mmol) was added to the mixture and stirred for 1 hour then another sodium tert-butoxide (77.315 mg, 0.8045 mmol) was added and the mixture was stirred for 1 more hour. The mixture was then cooled down to 0° C. and quenched by the addition of an aqueous solution of 1N HCl (5 mL). The biphasic mixture was stirred for 30 minutes. The layers were then separated, and the aqueous layer was extracted with 2-methyltetrahydrofuran (3×15 mL). The combined organic layers were dried over sodium sulfate, filtered, and concentrated in vacuo. The crude mixture was purified by reverse phase chromatography on a 50 g Cis GOLD cartridge, eluting with a gradient of 20 to 100% of MeOH in acidic water (0.1% of hydrochloric acid in water) to afford after evaporation 3-[[4-(2,6-dimethylphenyl)-6-[[6-(5-isopropoxypyrimidin-2-yl)-3,3-dimethyl-2-piperidyl]methoxy]pyrimidin-2-yl]sulfamoyl]benzoic acid (hydrochloride salt) (120 mg, 53%) as a white solid. ESI-MS m/z calc. 660.273, found 661.2 (M+1)⁺; Retention time: 1.44 minutes; LC method X.

### Step 15: 13-(2,6-dimethylphenyl)-4-(5-isopropoxypyrimidin-2-yl)-7,7-dimethyl-17,17-dioxo-10-oxa-17λ⁶-thia-3,14,16,23-tetrazatetracyclo[16.3.1.111,15.03,8]tricosa-1(21),11,13,15(23),18(22),19-hexaen-2-one (Compound 386)

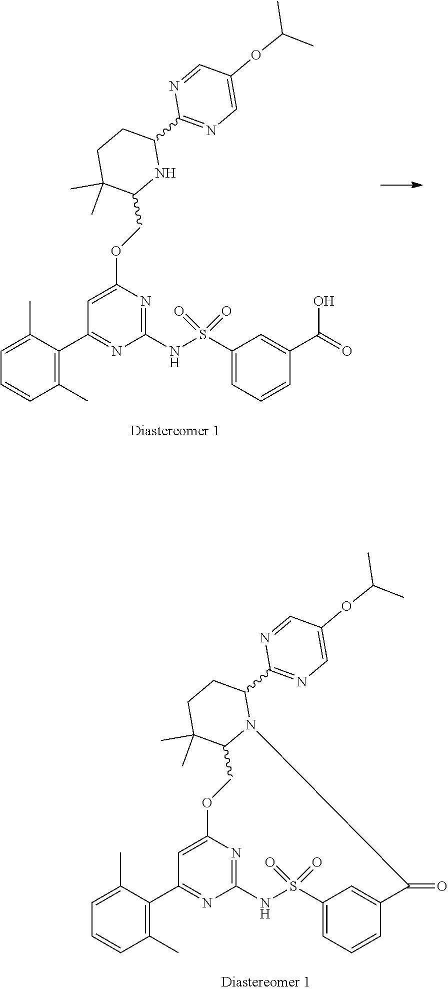 [SMILES: Cc1cccc(C)c1-c1cc(OCC2NC(c3ncc(OC(C)C)cn3)CCC2(C)C)nc(NS(=O)(=O)c2cccc(C(=O)O)c2)n1 ; Cc1cccc(C)c1-c1cc2nc(n1)NS(=O)(=O)c1cccc(c1)C(=O)N1C(c3ncc(OC(C)C)cn3)CCC(C)(C)C1CO2]

To a stirred solution of N-methylmorpholine (97.520 mg, 106 μL, 0.9641 mmol) in dimethylformamide (DMF) (19 mL) at 0° C. was added 2-chloro-4,6-dimethoxy-1,3,5-triazine (48 mg, 0.2734 mmol) followed by 3-[[4-(2,6-dimethylphenyl)-6-[[6-(5-isopropoxypyrimidin-2-yl)-3,3-dimethyl-2-piperidyl]methoxy]pyrimidin-2-yl]sulfamoyl]benzoic acid (hydrochloride salt) (90 mg, 0.1291 mmol) in dimethylformamide (DMF) (2 mL). The reaction mixture was stirred at 0° C. for 5 minutes. Then the reaction was warmed up to room temperature and stirred at room temperature for 24 hours. The reaction mixture was kept and combined with other reaction mixtures prepared separately

Crude mixtures of 13-(2,6-dimethylphenyl)-4-(5-isopropoxypyrimidin-2-yl)-7,7-dimethyl-17,17-dioxo-10-oxa-17λ⁶-thia-3,14,16,23-tetrazatetracyclo[16.3.1.111,15.03,8]tricosa-1(21),11,13,15(23),18(22),19-hexaen-2-one (92.9 mg, 0.1445 mmol) and 13-(2,6-dimethylphenyl)-4-(5-isopropoxypyrimidin-2-yl)-7,7-dimethyl-17,17-dioxo-10-oxa-17λ⁶-thia-3,14,16,23-tetrazatetracyclo[16.3.1.111,15.03,8]tricosa-1(21),11,13,15(23),18(22),19-hexaen-2-one (13.8 mg, 0.0215 mmol) were combined, evaporated under reduced pressure. The residue was purified by reverse phase chromatography on a 40 g Cis Gold cartridge using a gradient of MeCN (5% for 3.5 CV then to 100% over 21CV) in acidic water (formic acid=0.1% w/w). The fractions containing the product were combined and evaporated to dryness. The white solid was dissolved in CH₃CN/water and lyophilized to give the pure 13-(2,6-dimethylphenyl)-4-(5-isopropoxypyrimidin-2-yl)-7,7-dimethyl-17,17-dioxo-10-oxa-17λ⁶-thia-3,14,16,23-tetrazatetracyclo[16.3.1.111,15.03,8]tricosa-1(21),11,13,15(23),18(22),19-hexaen-2-one (40.9 mg, 37%) as a white solid. ¹H NMR (400 MHz, DMSO-d₆) δ 13.19-12.73 (m, 1H), 8.63 (s, 2H), 8.44 (br. s., 1H), 7.89 (br. s., 1H), 7.69 (br. s., 2H), 7.29-7.19 (m, 1H), 7.10 (d, J=7.1 Hz, 2H), 6.13 (d, J=4.6 Hz, 2H), 4.85 (quin, J 6.0 Hz, 1H), 4.69 (d, J=9.3 Hz, 1H), 3.48-3.35 (m, 2H), 2.71-2.63 (m, 1H, overlap with DMSO-D6 satellite), 2.14-1.92 (m, 8H), 1.33 (m, 7H), 0.84 (s, 3H), 0.79 (s, 3H). ESI-MS m/z calc. 642.26245, found 643.3 (M+1)⁺; Retention time: 3.38 minutes; LC method 1B.

### Example 144: Preparation of Compound 387

### Step 1: [6-(5-Isopropoxypyrimidin-2-yl)-3,3-dimethyl-2-piperidyl]methanol, diastereomer 2

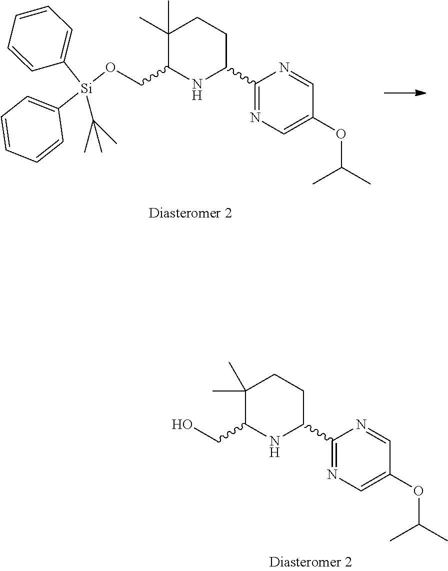 [SMILES: CC(C)Oc1cnc(C2CCC(C)(C)C(CO[Si](c3ccccc3)(c3ccccc3)C(C)(C)C)N2)nc1 ; CC(C)Oc1cnc(C2CCC(C)(C)C(CO)N2)nc1]

In a 50 mL flask was introduced tert-butyl-[[6-(5-isopropoxypyrimidin-2-yl)-3,3-dimethyl-2-piperidyl]methoxy]-diphenyl-silane diastereomer 2 (210 mg, 0.3853 mmol) and THE (3.1500 mL). The mixture was stirred for 10 min then TBAF (in THF) (1.16 mL of 1 M, 1.1600 mmol) was added at room temperature. After addition the mixture was warmed up to 40° C. and stirred at this temperature overnight. The reaction was cooled down to room temperature and the volatiles were removed by evaporation under reduced pressure. The crude was purified by reverse-phase chromatography on Cis (column: 30 g HP Gold Cis; gradient: 30 to 100% acetonitrile in water containing 0.1% of formic acid, 15 CV) to afford [6-(5-isopropoxypyrimidin-2-yl)-3,3-dimethyl-2-piperidyl]methanol (30 mg, 28%) as a light yellow oil. ESI-MS m/z calc. 279.19467, found 280.2 (M+1)⁺; Retention time: 1.2 minutes; LC method X.

### Step 2: 3-[[4-(2,6-Dimethylphenyl)-6-[[6-(5-isopropoxypyrimidin-2-yl)-3,3-dimethyl-2-piperidyl]methoxy]pyrimidin-2-yl]sulfamoyl]benzoic acid, diastereomer 2

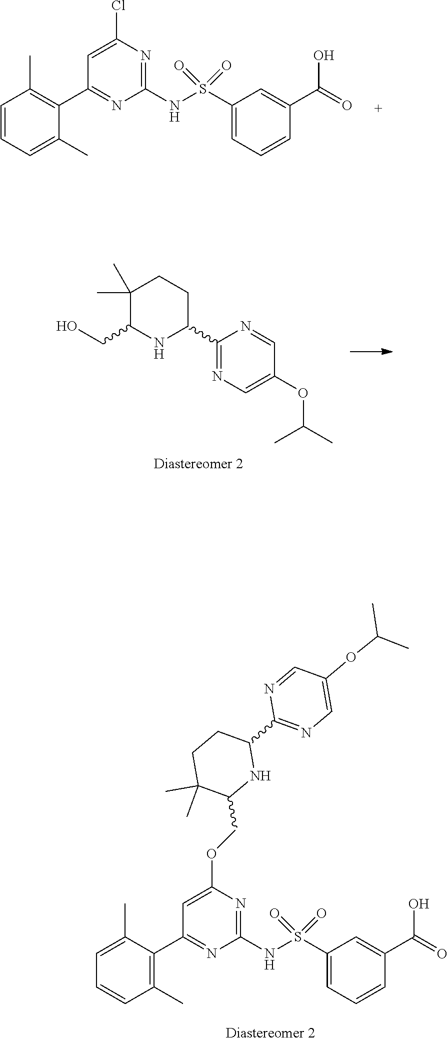 [SMILES: Cc1cccc(C)c1-c1cc(Cl)nc(NS(=O)(=O)c2cccc(C(=O)O)c2)n1 ; CC(C)Oc1cnc(C2CCC(C)(C)C(CO)N2)nc1 ; Cc1cccc(C)c1-c1cc(OCC2NC(c3ncc(OC(C)C)cn3)CCC2(C)C)nc(NS(=O)(=O)c2cccc(C(=O)O)c2)n1]

A solution of [6-(5-isopropoxypyrimidin-2-yl)-3,3-dimethyl-2-piperidyl]methanol (25 mg, 0.0895 mmol) in anhydrous N,N-dimethylformamide (125 μL) was added to a solution of 3-[[4-chloro-6-(2,6-dimethylphenyl)pyrimidin-2-yl]sulfamoyl]benzoic acid (41 mg, 0.0981 mmol) in 2-methyltetrahydrofuran (0.625 mL). The mixture was cooled down to 10-15° C. and then sodium tert-butoxide (56 mg, 0.5827 mmol) was added. The reaction was stirred at 10-15° C. for 2 hours and sodium tert-butoxide (56 mg, 0.5827 mmol) was added to the mixture and stirred for 1 hour. The mixture was then cooled down to 0° C. and quenched by the addition of an aqueous solution of 1N HCl (0.5 mL). The biphasic mixture was stirred for 30 minutes. The layers were then separated, and the aqueous layer was extracted with 2-methyltetrahydrofuran (3×15 mL). The layers were then separated and the aqueous layer was extracted with 2-methyltetrahydrofuran (3×15 mL). The combined organic layers were combined with another crude batch reaction for purification.

Crude 3-[[4-(2,6-dimethylphenyl)-6-[[6-(5-isopropoxypyrimidin-2-yl)-3,3-dimethyl-2-piperidyl]methoxy]pyrimidin-2-yl]sulfamoyl]benzoic acid (hydrochloride salt) (62.4 mg, 0.0895 mmol) and crude 3-[[4-(2,6-dimethylphenyl)-6-[[6-(5-isopropoxypyrimidin-2-yl)-3,3-dimethyl-2-piperidyl]methoxy]pyrimidin-2-yl]sulfamoyl]benzoic acid (hydrochloride salt) (12.48 mg, 0.0179 mmol) were combined to be purified by reverse flash chromatography on a 1.5 g C₁₈ Gold cartridge using a gradient of MeCN (20% for 3.5 CV then to 100% over 10 CV then 100% for 10 CV) in acidic water (HCl=0.1% w/w) to provide the pure 3-[[4-(2,6-dimethylphenyl)-6-[[6-(5-isopropoxypyrimidin-2-yl)-3,3-dimethyl-2-piperidyl]methoxy]pyrimidin-2-yl]sulfamoyl]benzoic acid diastereomer 2 (hydrochloride salt) (32 mg, 40%). ESI-MS m/z calc. 660.273, found 661.2 (M+1)⁺; Retention time: 1.44 minutes; LC method X.

### Step 3: 13-(2,6-Dimethylphenyl)-4-(5-isopropoxypyrimidin-2-yl)-7,7-dimethyl-17,17-dioxo-10-oxa-17λ⁶-thia-3,14,16,23-tetrazatetracyclo[16.3.1.111,15.03,8]tricosa-1(21),11,13,15(23),18(22),19-hexaen-2-one (Compound 387)

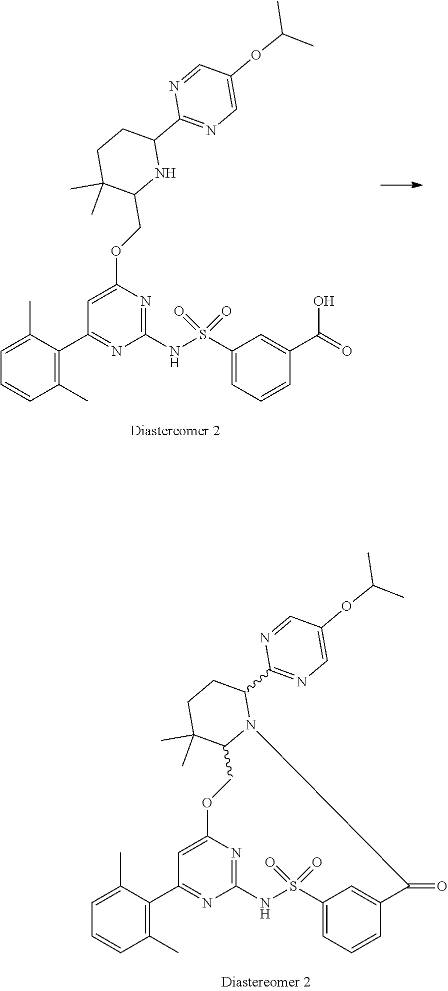 [SMILES: Cc1cccc(C)c1-c1cc(OCC2NC(c3ncc(OC(C)C)cn3)CCC2(C)C)nc(NS(=O)(=O)c2cccc(C(=O)O)c2)n1 ; Cc1cccc(C)c1-c1cc2nc(n1)NS(=O)(=O)c1cccc(c1)C(=O)N1C(c3ncc(OC(C)C)cn3)CCC(C)(C)C1CO2]

To a stirred solution of N-methylmorpholine (34.960 mg, 38 μL, 0.3456 mmol) in dimethylformamide (DMF) (6.4 mL) at 0° C. was added 2-chloro-4,6-dimethoxy-1,3,5-triazine (17 mg, 0.0968 mmol) followed by 3-[[4-(2,6-dimethylphenyl)-6-[[6-(5-isopropoxypyrimidin-2-yl)-3,3-dimethyl-2-piperidyl]methoxy]pyrimidin-2-yl]sulfamoyl]benzoic acid diastereomer 2 (hydrochloride salt) (32 mg, 0.0459 mmol) in dimethylformamide (DMF) (0.64 mL). The reaction mixture was stirred at 0° C. for 5 minutes. Then the reaction was warmed up to room temperature and stirred at room temperature for 24 hours. The volatiles were removed under reduced pressure and the residue was subsequently purified by reverse flash chromatography on a 15.5 g C₁₈ Gold cartridge using a gradient of MeCN (10% for 3.5 CV then to 100% over 20 CV then 100% for 3.5 CV) in acidic water (formic acid=0.1% w/w) to provide the pure 13-(2,6-dimethylphenyl)-4-(5-isopropoxypyrimidin-2-yl)-7,7-dimethyl-17,17-dioxo-10-oxa-17λ⁶-thia-3,14,16,23-tetrazatetracyclo[16.3.1.111,15.03,8]tricosa-1(21),11,13,15(23),18(22),19-hexaen-2-one diastereomer 2 (5.6 mg, 18%) as a white solid. ¹H NMR (400 MHz, DMSO-d₆) δ 12.95 (br. s, 1H), 8.63 (s, 2H), 8.44 (s, 1H), 7.89 (br. s, 1H), 7.69 (br. s, 2H), 7.28-7.20 (m, 1H), 7.10 (d, J=7.3 Hz, 2H), 6.13 (d, J=4.4 Hz, 2H), 4.85 (spt, J=6.0 Hz, 1H), 4.69 (d, J=9.0 Hz, 1H), 3.47-3.35 (m, 2H), 2.71-2.64 (m, 1H), 2.17-1.86 (m, 8H), 1.40-1.29 (m, 7H), 0.84 (s, 3H), 0.79 (s, 3H). ESI-MS m/z calc. 642.26245, found 643.2 (M+1)⁺; Retention time: 4.61 minutes; LC method Z.

### Example 145: Preparation of Compound 388 and Compound 389

### Step 1: 01-tert-Butyl 06-methyl 3,4-dihydro-2H-pyridine-1,6-dicarboxylate

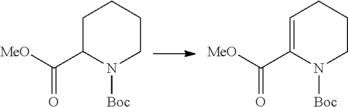 [SMILES: COC(=O)C1CCCCN1C(=O)OC(C)(C)C ; COC(=O)C1=CCCCN1C(=O)OC(C)(C)C]

Into a solution of 01-tert-butyl 02-methyl piperidine-1,2-dicarboxylate (65 g, 259.15 mmol) in anhydrous THE (500 mL) was added 1.0 M LiHMDS in THE (400 mL of 1 M, 400.00 mmol) at −78° C. The reaction mixture was stirred at the same temperature for 30 minutes. To a stirred solution of phenylselenyl chloride (150 g, 783.22 mmol) in anhydrous THE (1000 mL) was added the solution of lithium enolate prepared above at −78° C. through a cannular. The reaction was slowly raised to rt and stirred overnight. The solvent was removed under vacuum. The residue was diluted with saturated ammonium chloride (500 mL) and DCM (500 mL). After phase separation, the aqueous layer was extracted with DCM (2×400 mL). The combined organic layers were washed with brine (300 mL), dried over anhydrous sodium sulfate and concentrated under vacuum. The residue was purified by silica gel chromatography using 0 to 15% ethyl acetate in hexane to furnish 01-tert-butyl 06-methyl 3,4-dihydro-2H-pyridine-1,6-dicarboxylate (39.2 g, 60%) as a yellow oil, which solidified upon standing. ESI-MS m/z calc. 241.13141, found 242.0 (M+1)⁺; Retention time: 2.83 minutes; LC method T.

### Step 2: 01-tert-Butyl 02-methyl 3-isopropylpiperidine-1,2-dicarboxylate

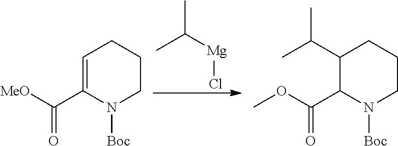

An oven dried 3 neck flask attached with a thermometer (to monitor internal temperature) was charged with CuI (32 g, 168.02 mmol) under argon. Anhydrous THE (95.2 mL) was added to it and the resulting suspension was cooled to −78° C. (dry ice acetone bath). chloro(isopropyl)magnesium in THE (167.00 mL of 2 M, 334.00 mmol) in THE was added to the reaction mixture at −78° C. (dry ice acetone bath) over 45 min. After 10 min, the reaction mixture was transferred to −35° C. bath (acetone in controlled amount of dry ice and monitored using thermometer) and was stirred for 30 minutes. The temperature was brought back down to −78° C. bath and stirred for 10 min. A solution of O1-tert-butyl O6-methyl 3,4-dihydro-2H-pyridine-1,6-dicarboxylate (8 g, 31.498 mmol) in anhydrous THE (95.2 mL) was added (at −78° C.) to the reaction mixture dropwise over 1 h 10 min. The reaction was then stirred at −78° C. for 1 h and then the temperature was gradually raised to −10° C. and stirred for another 30 min. The SM was completely consumed at this time by LCMS, a peak corresponding to product mass (TIC only, no UV) was observed in LCMS and TLC (5:1 hexanes: EtOAc and staining with ninhydrin, product appears as deep blue color (other impurities/side products shows purple and yellow color)). The reaction was quenched with saturated ammonium chloride (300 mL) at −10° C. and diluted with EtOAc (300 ml). The insoluble material was filtered off through a pad of Celite washing thoroughly with EtOAc. The aqueous layer was extracted with ethyl acetate (2×300 mL). The combined organic layers were washed with brine (2×250 ml). The crude product was purified by flash chromatography (Combiflash, loaded onto dry silica in 330 g cartridge using 0-15% diethyl ether in hexanes as eluent over 60 min) to provide 01-tert-butyl 02-methyl 3-isopropylpiperidine-1,2-dicarboxylate (5.9 g, 62%) as light yellow oil. ¹H NMR (500 MHz, Chloroform-d) δ 5.01-4.79 (m, 1H), 4.12-3.79 (m, 1H), 3.73 (s, 3H), 3.14-2.75 (m, 1H), 1.86-1.62 (m, 3H), 1.63-1.52 (m, 1H), 1.45 (s, 9H), 1.39-1.28 (m, 2H), 1.03 (d, J=5.8 Hz, 3H), 0.92 (d, J=6.1 Hz, 3H). ESI-MS m/z calc. 285.194, found 286.5 (M+1)⁺; Retention time: 6.16 minutes; LC method S.

### Step 3: tert-Butyl 2-(hydroxymethyl)-3-isopropyl-piperidine-1-carboxylate

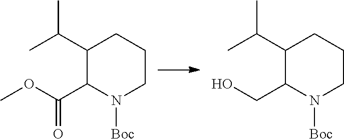 [SMILES: COC(=O)C1C(C(C)C)CCCN1C(=O)OC(C)(C)C ; CC(C)C1CCCN(C(=O)OC(C)(C)C)C1CO]

Into a solution of 01-tert-butyl 02-methyl 3-isopropylpiperidine-1,2-dicarboxylate (5.84 g, 19.441 mmol) in anhydrous toluene (117 mL) was added 1.0 M DIBAL-H in toluene (52 mL of 1 M, 52.000 mmol) dropwise over 15 min at 0° C. under nitrogen. The reaction was stirred at the same temperature for another 45 min. The starting material was fully consumed at this time by LCMS. The reaction was quenched with sat Na,K-tartrate (aq.) (250 mL) at 0° C. and diluted with ethyl acetate (200 mL). The reaction mixture was then brought to rt and stirred for 30 min. The solid residue was filtered a small pad of celite washing thoroughly with EtOAc. The aqueous layer was extracted with ethyl acetate (2×100 mL). The combined organic layers were washed with brine (250 mL), dried over anhydrous sodium sulfate and concentrated under vacuum to furnish crude tert-butyl 2-(hydroxymethyl)-3-isopropyl-piperidine-1-carboxylate (4.9 g, 88%) as a clear liquid which was used in the next Step without further purification. ¹H NMR (500 MHz, Chloroform-d) δ 4.45-4.17 (m, 1H), 3.91-3.79 (m, 1H), 3.59 (dd, J=11.5, 5.1 Hz, 1H), 3.02-2.72 (m, 1H), 1.82-1.69 (m, 2H), 1.69-1.53 (m, 3H), 1.46 (s, 9H), 1.44-1.33 (m, 1H), 1.18-1.10 (m, 1H), 1.00 (dd, J=6.5, 1.21 Hz, 3H), 0.89 (dd, J=6.7, 1.1 Hz, 3H). ESI-MS m/z calc. 257.1991, found 258.3 (M+1)⁺; Retention time: 4.78 minutes; LC method S.

### Step 4: tert-Butyl 2-[[tert-butyl(diphenyl)silyl]oxymethyl]-3-isopropyl-piperidine-1-carboxylate

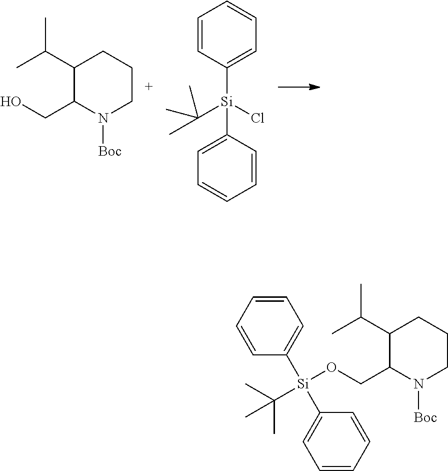

A solution of tert-butyl 2-(hydroxymethyl)-3-isopropyl-piperidine-1-carboxylate (3.5 g, 12.511 mmol) in anhydrous DCM (80 mL) was cooled to 0° C. under nitrogen. imidazole (2.2 g, 32.316 mmol) was added to the solution. After 5 min, tert-butyl 2-(hydroxymethyl)-3-isopropyl-piperidine-1-carboxylate (3.5 g, 12.511 mmol) was added dropwise at 0° C. to the reaction mixture. The reaction was stirred at 0° C. for 30 min followed by stirring at room temperature for 1 hour. The reaction was diluted with DCM (150 mL) and brine (150 mL). The aqueous layer was then extracted with DCM (2×150 ml). The combined organic solution was dried over anhydrous sodium sulfate, filtered, and concentrated under vacuum. The crude product was purified by flash chromatography (Combiflash, loaded onto dry silica in 120 g cartridge using 0-15% diethyl ether in hexanes as eluent) and the solvent was removed to provide tert-butyl 2-[[tert-butyl(diphenyl)silyl]oxymethyl]-3-isopropyl-piperidine-1-carboxylate (5.3 g, 81%) as colorless gel. ESI-MS m/z calc. 495.31686, found 496.6 (M+1)⁺; Retention time: 9.74 minutes; LC method S.

### Step 5: tert-Butyl 2-[[tert-butyl(diphenyl)silyl]oxymethyl]-3-isopropyl-6-oxo-piperidine-1-carboxylate

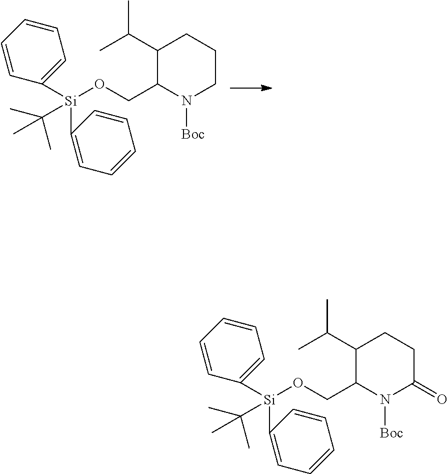 [SMILES: CC(C)C1CCCN(C(=O)OC(C)(C)C)C1CO[Si](c1ccccc1)(c1ccccc1)C(C)(C)C ; CC(C)C1CCC(=O)N(C(=O)OC(C)(C)C)C1CO[Si](c1ccccc1)(c1ccccc1)C(C)(C)C]

Into a solution of tert-butyl 2-[[tert-butyl(diphenyl)silyl]oxymethyl]-3-isopropyl-piperidine-1-carboxylate (2.6 g, 5.2444 mmol) in a solvent mixture of CCl₄ (14.5 mL), ACN (14.5 mL) and water (19.3 mL) was added NaIO₄ (12 g, 56.103 mmol) and Ruthenium(III) chloride hydrate (120 mg, 0.5785 mmol) at 0° C. The reaction was stirred at 0° C. for 30 minutes, then raised to rt and stirred for 2 hours. The reaction was then diluted with ethyl acetate (50 mL). The solid was filtered through a pad of Celite washing thoroughly with EtOAc. The filtrate was concentrated under reduced pressure. The crude product was purified by flash chromatography (Combiflash, loaded onto dry silica in 80 g cartridge using 0-15% EtOAc in hexanes as eluent over 40 min) and the solvent was removed to provide tert-butyl 2-[[tert-butyl(diphenyl)silyl]oxymethyl]-3-isopropyl-6-oxo-piperidine-1-carboxylate (1.2 g, 44%) as colorless gel. ESI-MS m/z calc. 509.29614, found 510.5 (M+1)⁺; Retention time: 8.8 minutes; LC method S.

### Step 6: tert-Butyl 2-[[tert-butyl(diphenyl)silyl]oxymethyl]-3-isopropyl-6-(trifluoromethylsulfonyloxy)-3,4-dihydro-2H-pyridine-1-carboxylate

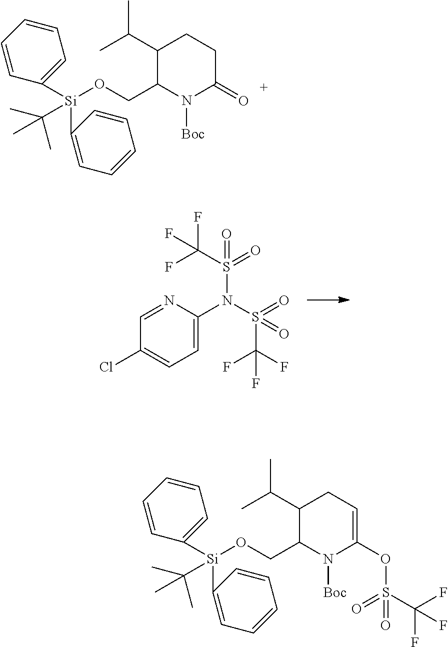 [SMILES: CC(C)C1CCC(=O)N(C(=O)OC(C)(C)C)C1CO[Si](c1ccccc1)(c1ccccc1)C(C)(C)C ; O=S(=O)(N(c1ccc(Cl)cn1)S(=O)(=O)C(F)(F)F)C(F)(F)F ; CC(C)C1CC=C(OS(=O)(=O)C(F)(F)F)N(C(=O)OC(C)(C)C)C1CO[Si](c1ccccc1)(c1ccccc1)C(C)(C)C]

A solution of tert-butyl 2-[[tert-butyl(diphenyl)silyl]oxymethyl]-3-isopropyl-6-oxo-piperidine-1-carboxylate (3.7 g, 6.8955 mmol) in anhydrous THE (74 mL) was cooled at −78° C. under nitrogen. 1.0 M LiHMDS in THE (8.5 mL of 1.0 M, 8.5000 mmol) was added dropwise over 25 min at −78° C. The reaction was stirred at the same temperature for 0.5 hour. A solution of N-(5-chloro-2-pyridyl)-1,1,1-trifluoro-N-(trifluoromethylsulfonyl)methanesulfonamide (3.3 g, 8.4037 mmol) in anhydrous THF (8.2 mL) was added to the reaction mixture dropwise over 15 min. The reaction was then stirred at −78° C. for 1 hour. The reaction mixture was then warmed up to −40° C. and stirred for 15 min. The reaction was quenched with saturated sodium bicarbonate (15 mL) at −40° C. and then slowly raised to rt. The aqueous solution was extracted with diethyl ether (3×50 mL). The combined organic layers were washed with brine (20 mL), dried over anhydrous magnesium sulfate, filtered, and concentrated under vacuum. The crude product was purified by flash chromatography (Combiflash, loaded onto dry silica in 4 g cartridge using 0-10% EtOAc in hexanes as eluent) to provide tert-butyl 2-[[tert-butyl(diphenyl)silyl]oxymethyl]-3-isopropyl-6-(trifluoromethylsulfonyloxy)-3,4-dihydro-2H-pyridine-1-carboxylate (223 mg, 79%) as colorless gel. ¹H NMR (500 MHz, Chloroform-d) δ 7.74-7.49 (m, 4H), 7.46-7.33 (m, 6H), 5.09 (t, J=4.3 Hz, 1H), 4.72-4.46 (m, 1H), 3.77 (dd, J=10.4, 6.9 Hz, 1H), 3.50 (dd, J=10.3, 7.6 Hz, 1H), 2.18-2.03 (m, 1H), 2.00 (dd, J=7.1, 4.6 Hz, 1H), 1.66-1.58 (m, 1H), 1.48 (s, 9H), 1.33-1.23 (m, 1H), 1.04 (s, 9H), 0.92 (dd, J=6.6, 1.3 Hz, 3H), 0.90 (d, J=6.6 Hz, 3H) ESI-MS m/z calc. 641.2454, found 642.7 (M+1)⁺; Retention time: 9.64 minutes; LC method S.

### Step 7: 2-Chloro-5-isopropoxy-pyrimidine

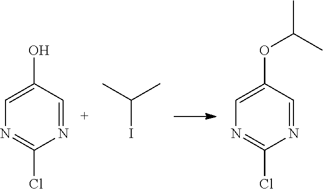

A stirred mixture of 2-chloropyrimidin-5-ol (40 g, 306.44 mmol), 2-iodopropane (80 g, 470.61 mmol) and potassium carbonate (100 g, 723.56 mmol) in DMF (400.00 mL) was heated at 80° C. for 2 h. The reaction mixture was allowed to cool to rt and then water (1 L) was added. The solids were filtered, washed with water and dissolved in EtOAc (1 L). The ethyl acetate layer was dried over sodium sulfate, filtered, and concentrated in vacuo to give 2-chloro-5-isopropoxy-pyrimidine (42 g, 75%) as a white solid. ESI-MS m/z calc. 172.04034, found 173.3 (M+1)⁺; Retention time: 3.35 minutes; LC method S.

### Step 7: tert-Butyl 2-[[tert-butyl(diphenyl)silyl]oxymethyl]-3-isopropyl-6-(4,4,5,5-tetramethyl-1,3,2-dioxaborolan-2-yl)-3,4-dihydro-2H-pyridine-1-carboxylate

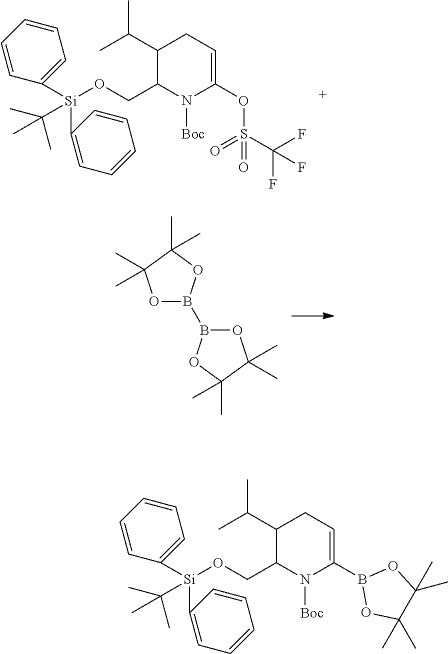

A 250 ml flask was charged with 4,4,5,5-tetramethyl-2-(4,4,5,5-tetramethyl-1,3,2-dioxaborolan-2-yl)-1,3,2-dioxaborolane (2.3 g, 9.0573 mmol), tert-butyl 2-[[tert-butyl(diphenyl)silyl]oxymethyl]-3-isopropyl-6-(trifluoromethylsulfonyloxy)-3,4-dihydro-2H-pyridine-1-carboxylate (3.9 g, 5.7727 mmol), potassium carbonate (2.1 g, 15.195 mmol), Pd(PPh3)2Cl2 (406 mg, 0.5784 mmol), triphenylphosphine (303 mg, 1.1552 mmol) under argon atmosphere. A pre-degassed 1,4-dioxane (62.5 mL) (bubbling argon for 2 h with stirring) was added to the reaction flask. The reaction mixture was further degassed with argon for 35 minutes. The reaction was then heated at 90° C. for 5 hours 20 min. The complete consumption of the starting material was observed by LCMS. The reaction was cooled to room temperature. The reaction was diluted with ethyl acetate (150 mL) and filtered through a pad of celite washing thoroughly with EtOAc. Brine (150 ml) was added to the filtrate. Two layers were separated, and the aqueous layer was extracted with ethyl acetate (2×100 mL). The combined organic layers were dried over anhydrous sodium sulfate and concentrated under vacuum. The crude product was purified by flash chromatography (Combiflash, loaded with minimum amount of benzene in 120 g cartridge using 0-15% EtOAc in hexanes as eluent) and the solvent was removed to provide tert-butyl 2-[[tert-butyl(diphenyl)silyl]oxymethyl]-3-isopropyl-6-(4,4,5,5-tetramethyl-1,3,2-dioxaborolan-2-yl)-3,4-dihydro-2H-pyridine-1-carboxylate (2.79 g, 74%) as a white foam. ¹H NMR (500 MHz, Chloroform-d) δ 7.70-7.50 (m, 4H), 7.45-7.32 (m, 6H), 5.00 (t, J=3.8 Hz, 1H), 4.27-4.17 (m, 1H), 3.62 (dd, J=9.8, 5.6 Hz, 1H), 3.56 (t, J=9.6 Hz, 1H), 2.15-1.90 (m, 2H), 1.82-1.71 (m, 1H), 1.57-1.53 (m, 1H), 1.41 (s, 9H), 1.24 (d, J=3.9 Hz, 12H), 1.04 (s, 9H), 0.91 (d, J=3.1 Hz, 3H), 0.90 (d, J=3.2 Hz, 3H). ESI-MS m/z calc. 619.3864, found 620.8 (M+1)⁺; Retention time: 4.55 minutes; LC method W.

### Step 8: tert-Butyl 2-[[tert-butyl(diphenyl)silyl]oxymethyl]-6-(5-isopropoxypyrimidin-2-yl)-3-isopropyl-3,4-dihydro-2H-pyridine-1-carboxylate

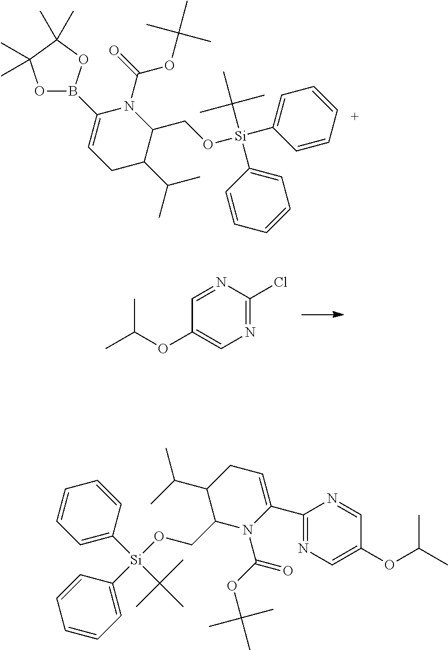 [SMILES: CC(C)C1CC=C(B2OC(C)(C)C(C)(C)O2)N(C(=O)OC(C)(C)C)C1CO[Si](c1ccccc1)(c1ccccc1)C(C)(C)C ; CC(C)Oc1cnc(Cl)nc1 ; CC(C)Oc1cnc(C2=CCC(C(C)C)C(CO[Si](c3ccccc3)(c3ccccc3)C(C)(C)C)N2C(=O)OC(C)(C)C)nc1]

To a vial was added tert-butyl 2-[[tert-butyl(diphenyl)silyl]oxymethyl]-3-isopropyl-6-(4,4,5,5-tetramethyl-1,3,2-dioxaborolan-2-yl)-3,4-dihydro-2H-pyridine-1-carboxylate (365 mg, 0.5890 mmol), 2-chloro-5-isopropoxy-pyrimidine (161 mg, 0.8861 mmol), diacetoxypalladium (26.5 mg, 0.1180 mmol), triphenylphosphane (61.8 mg, 0.2356 mmol), and phosphate (Potassium Ion (3)) (375 mg, 1.767 mmol). dioxane (7.3 mL) and water (730 μL) were added to the reaction mixture and the solution was sparged with nitrogen for 10 minutes. The vial was flushed with argon, sealed with a screw cap and heated at 1000 C for 5 hours. The reaction mixture was cooled to room temperature and partitioned between ethyl acetate and water. The layers were separated, and the organic layer was dried over sodium sulfate, filtered and concentrated under reduced pressure. The crude product was purified by silica gel column chromatography using a gradient of 0-20% ethyl acetate in hexanes to provide tert-butyl 2-[[tert-butyl(diphenyl)silyl]oxymethyl]-6-(5-isopropoxypyrimidin-2-yl)-3-isopropyl-3,4-dihydro-2H-pyridine-1-carboxylate (180 mg, 49%) as a white solid. ESI-MS m/z calc. 629.36487, found 630.9 (M+1)⁺; Retention time: 0.86 minutes; LC method D (50-99% gradient).

### Step 9: tert-Butyl-[[6-(5-isopropoxypyrimidin-2-yl)-3-isopropyl-2-piperidyl]methoxy]-diphenyl-silane

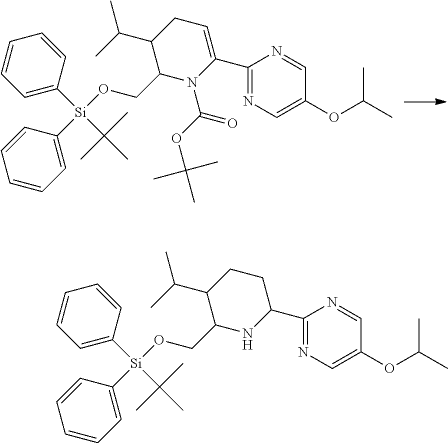 [SMILES: CC(C)Oc1cnc(C2=CCC(C(C)C)C(CO[Si](c3ccccc3)(c3ccccc3)C(C)(C)C)N2C(=O)OC(C)(C)C)nc1 ; CC(C)Oc1cnc(C2CCC(C(C)C)C(CO[Si](c3ccccc3)(c3ccccc3)C(C)(C)C)N2)nc1]

tert-Butyl 2-[[tert-butyl(diphenyl)silyl]oxymethyl]-6-(5-isopropoxypyrimidin-2-yl)-3-isopropyl-3,4-dihydro-2H-pyridine-1-carboxylate (145 mg, 0.2302 mmol) was dissolved in DCM (2 mL) and cooled in an ice water bath. TFA (850 μL, 11.03 mmol) was added to the reaction, the cooling bath was removed after 5 minutes and the reaction was warmed to room temperature and continued to stir at this temperature for 1 hour. The reaction was then concentrated under reduced pressure to provide intermediate tert-butyl-[[6-(5-isopropoxypyrimidin-2-yl)-3-isopropyl-1,2,3,4-tetrahydropyridin-2-yl]methoxy]-diphenyl-silane (trifluoroacetate salt). ESI-MS m/z calc. 529.31244, found 530.8 (M+1)⁺; Retention time: 0.75 minutes. The resulting intermediate was then dissolved in THE (4 mL) followed by the addition of triethylamine (65 μL, 0.4664 mmol) and then sodium triacetoxyborohydride (195 mg, 0.9201 mmol). The reaction mixture was stirred at room temperature for 16 hours. The reaction mixture was quenched with aqueous HCl (384 μL of 6 M, 2.304 mmol) and then purified by reverse phase HPLC using (10-99% ACN/water (5 mM HCl)). The desired factions were combined and concentrated under reduced pressure to provide tert-butyl-[[6-(5-isopropoxypyrimidin-2-yl)-3-isopropyl-2-piperidyl]methoxy]-diphenyl-silane (hydrochloride salt) (95.7 mg, 73%). ESI-MS m/z calc. 531.3281, found 532.9 (M+1)⁺; Retention time: 0.72 minutes; LC method E.

### Step 10: [6-(5-Isopropoxypyrimidin-2-yl)-3-isopropyl-2-piperidyl]methanol

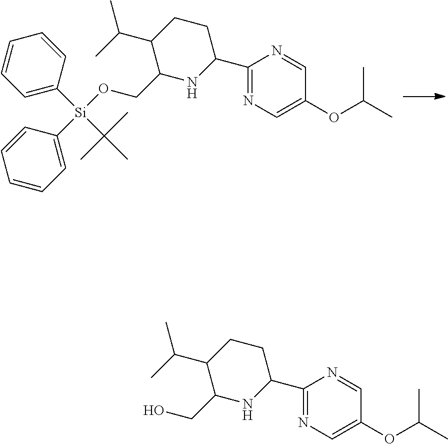

tert-Butyl-[[6-(5-isopropoxypyrimidin-2-yl)-3-isopropyl-2-piperidyl]methoxy]-diphenyl-silane (hydrochloride salt) (90 mg, 0.1584 mmol) was dissolved in dioxane (1.5 mL) followed by the addition of aqueous HCl (655 μL of 12.1 M, 7.926 mmol). The reaction mixture was heated at 100° C. for 1 h. The reaction mixture was cooled to room temperature, and purified by reverse phase HPLC using (1-99% ACN/water (5 mM HCl)) to provide [6-(5-isopropoxypyrimidin-2-yl)-3-isopropyl-2-piperidyl]methanol (hydrochloride salt) (39 mg, 75%). ESI-MS m/z calc. 293.21033, found 294.3 (M+1)⁺; Retention time: 0.4 minutes; LC method D.

### Step 11: 3-[[4-(2,6-Dimethylphenyl)-6-[[6-(5-isopropoxypyrimidin-2-yl)-3-isopropyl-2-piperidyl]methoxy]pyrimidin-2-yl]sulfamoyl]benzoic acid

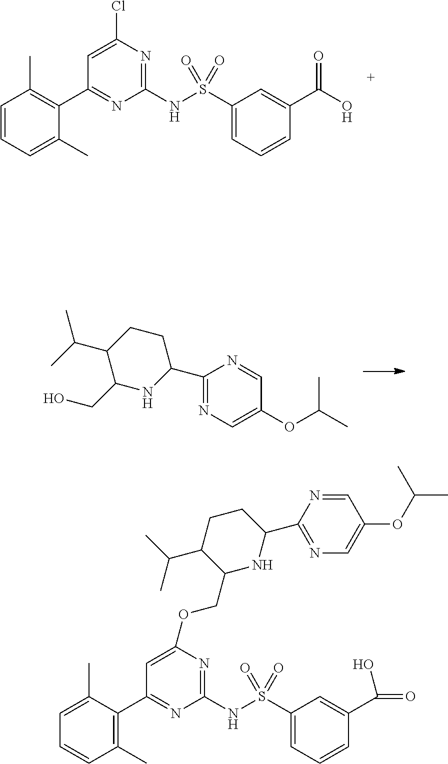 [SMILES: Cc1cccc(C)c1-c1cc(Cl)nc(NS(=O)(=O)c2cccc(C(=O)O)c2)n1 ; CC(C)Oc1cnc(C2CCC(C(C)C)C(CO)N2)nc1 ; Cc1cccc(C)c1-c1cc(OCC2NC(c3ncc(OC(C)C)cn3)CCC2C(C)C)nc(NS(=O)(=O)c2cccc(C(=O)O)c2)n1]

3-[[4-Chloro-6-(2,6-dimethylphenyl)pyrimidin-2-yl]sulfamoyl]benzoic acid (49 mg, 0.1173 mmol) was dissolved in THE (2 mL) followed by the addition of [6-(5-isopropoxypyrimidin-2-yl)-3-isopropyl-2-piperidyl]methanol (hydrochloride salt) (38.7 mg, 0.1173 mmol) and then sodium tert-butoxide (78.9 mg, 0.8210 mmol). The reaction was stirred at room temperature for 2 hours. The reaction mixture was cooled in an ice water bath and quenched with HCl (196 μL of 6 M, 1.176 mmol). The reaction mixture was warmed to room temperature, diluted with DMSO (1 mL), syringe filtered and purified by reverse phase HPLC using (1-70% ACN/water (5 mM HCl)) to provide 3-[[4-(2,6-dimethylphenyl)-6-[[6-(5-isopropoxypyrimidin-2-yl)-3-isopropyl-2-piperidyl]methoxy]pyrimidin-2-yl]sulfamoyl]benzoic acid (hydrochloride salt) (53.3 mg, 64%) as a white solid. ESI-MS m/z calc. 674.28864, found 675.6 (M+1)⁺; Retention time: 0.54 minutes; LC method D.

### Step 12: 13-(2,6-Dimethylphenyl)-4-(5-isopropoxypyrimidin-2-yl)-7-isopropyl-17,17-dioxo-10-oxa-17λ⁶-thia-3,14,16,23-tetrazatetracyclo[16.3.1.111,15.03,8]tricosa-1(21),11,13,15(23),18(22),19-hexaen-2-one

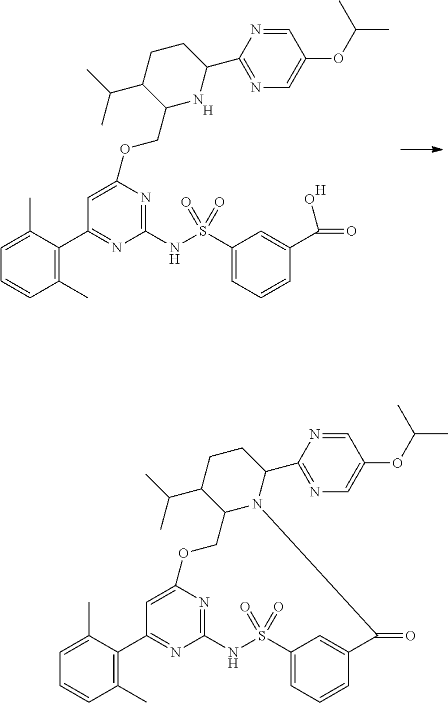

3-[[4-(2,6-Dimethylphenyl)-6-[[6-(5-isopropoxypyrimidin-2-yl)-3-isopropyl-2-piperidyl]methoxy]pyrimidin-2-yl]sulfamoyl]benzoic acid (hydrochloride salt) (48.3 mg, 0.06791 mmol) was dissolved in DMF (4.5 mL) followed by the addition of 2-chloro-4,6-dimethoxy-1,3,5-triazine (15.5 mg, 0.08828 mmol) and 4-methylmorpholine (52.3 μL, 0.4757 mmol). The reaction was stirred for 12 hours at room temperature. The reaction mixture was syringe filtered and purified by reverse phase HPLC using (10-99% ACN/water (5 mM HCl)) to provide 13-(2,6-dimethylphenyl)-4-(5-isopropoxypyrimidin-2-yl)-7-isopropyl-17,17-dioxo-10-oxa-17λ⁶-thia-3,14,16,23-tetrazatetracyclo[16.3.1.111,15.03,8]tricosa-1(21),11,13,15(23),18(22),19-hexaen-2-one (33.7 mg, 74%) as a white solid. ESI-MS m/z calc. 656.2781, found 657.5 (M+1)⁺; Retention time: 1.97 minutes; LC method A.

### Step 13: 13-(2,6-Dimethylphenyl)-4-(5-isopropoxypyrimidin-2-yl)-7-isopropyl-17,17-dioxo-10-oxa-17λ⁶-thia-3,14,16,23-tetrazatetracyclo[16.3.1.111,15.03,8]tricosa-1(21),11,13,15(23),18(22),19-hexaen-2-one, isomer 1 (Compound 388) and 13-(2,6-dimethylphenyl)-4-(5-isopropoxypyrimidin-2-yl)-7-isopropyl-17,17-dioxo-10-oxa-17λ⁶-thia-3,14,16,23-tetrazatetracyclo[16.3.1.111,15.03,8]tricosa-1(21),11,13,15(23),18(22),19-hexaen-2-one, isomer 2 (Compound 389)

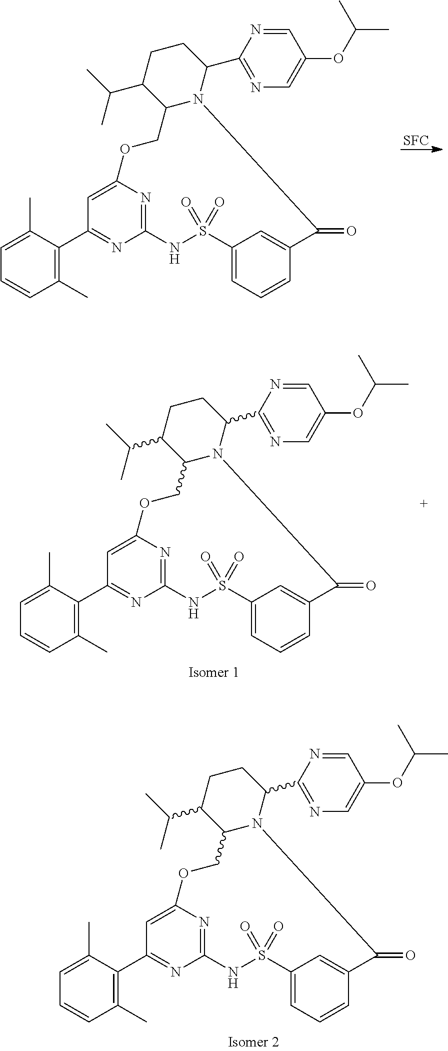 [SMILES: Cc1cccc(C)c1-c1cc2nc(n1)NS(=O)(=O)c1cccc(c1)C(=O)N1C(c3ncc(OC(C)C)cn3)CCC(C(C)C)C1CO2 ; "SFC" ; Cc1cccc(C)c1-c1cc2nc(n1)NS(=O)(=O)c1cccc(c1)C(=O)N1C(c3ncc(OC(C)C)cn3)CCC(C(C)C)C1CO2 ; Cc1cccc(C)c1-c1cc2nc(n1)NS(=O)(=O)c1cccc(c1)C(=O)N1C(c3ncc(OC(C)C)cn3)CCC(C(C)C)C1CO2]

13-(2,6-Dimethylphenyl)-4-(5-isopropoxypyrimidin-2-yl)-7-isopropyl-17,17-dioxo-10-oxa-17λ⁶-thia-3,14,16,23-tetrazatetracyclo[16.3.1.111,15.03,8]tricosa-1(21),11,13,15(23),18(22),19-hexaen-2-one (30 mg, 0.04522 mmol) was submitted for SFC separation of the isomers. The enantiomers were separated by chiral SFC using a ChiralPak IG column (250×10 mm; Sum) at 50° C. Mobile phase was a gradient 36 to 44% MeOH (w/20 mM NH₃), 64 to 56% CO₂ at a 10 mL/min flow. Concentration of the sample was 20.0 mg/mL in methanol (no modifier), injection volume 70 μL with an outlet pressure of 173 to 191 bar, detection wavelength of 210 nm to provide isomer 1, peak 1: 13-(2,6-dimethylphenyl)-4-(5-isopropoxypyrimidin-2-yl)-7-isopropyl-17,17-dioxo-10-oxa-17λ⁶-thia-3,14,16,23-tetrazatetracyclo[16.3.1.111,15.03,8]tricosa-1(21),11,13,15(23),18(22),19-hexaen-2-one (6 mg, 40%), ESI-MS m/z calc. 656.2781, found 657.5 (M+1)⁺; Retention time: 1.97 minutes, and isomer 2, peak 2: 13-(2,6-dimethylphenyl)-4-(5-isopropoxypyrimidin-2-yl)-7-isopropyl-17,17-dioxo-10-oxa-17λ⁶-thia-3,14,16,23-tetrazatetracyclo[16.3.1.111,15.03,8]tricosa-1(21),11,13,15(23),18(22),19-hexaen-2-one (5 mg, 33%), ESI-MS m/z calc. 656.2781, found 657.5 (M+1)⁺; Retention time: 1.97 minutes; LC method A.

### Example 146: Preparation of Compound 390 and Compound 391

### Step 1: 6-Bromo-N-methyl-N-spiro[2.3]hexan-5-yl-pyridin-2-amine

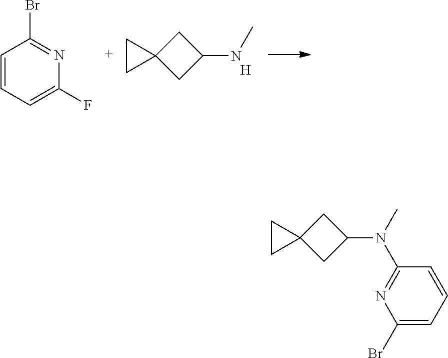

To a vial was added 2-bromo-6-fluoro-pyridine (984.6 mg, 5.595 mmol), N-methylspiro[2.3]hexan-5-amine (hydrochloride salt) (991.3 mg, 6.714 mmol), potassium carbonate (3.1 g, 22.43 mmol), followed by the addition of NMP (2 mL). The vial was sealed with a screw cap and the reaction mixture was stirred at 95° C. for 6 hours. The reaction mixture was cooled to room temperature and by reverse phase HPLC using (30-100% ACN/water (5 mM HCl)). The desired fractions were pooled, and partitioned between ethyl acetate and aqueous saturated sodium bicarbonate solution. The layers were separated and the organic layer was dried over sodium sulfate, filtered and concentrated under reduced pressure to provide 6-bromo-N-methyl-N-spiro[2.3]hexan-5-yl-pyridin-2-amine (1.35 g, 90%) as a clear oil. ¹H NMR (400 MHz, Chloroform-d) δ 7.25 (dd, J=8.4, 7.4 Hz, 1H), 6.70 (d, J=7.4 Hz, 1H), 6.43 (d, J=8.4 Hz, 1H), 4.97 (p, J=8.1 Hz, 1H), 3.04 (s, 3H), 2.47 (ddd, J=10.0, 8.2, 2.7 Hz, 2H), 2.23 (ddd, J=10.1, 8.1, 2.8 Hz, 2H), 0.58-0.49 (m, 2H), 0.47-0.38 (m, 2H). ESI-MS m/z calc. 266.04187, found 267.09 (M+1)⁺; Retention time: 0.8 minutes; LC method D.

### Step 2: tert-Butyl 2-[[tert-butyl(diphenyl)silyl]oxymethyl]-3-isopropyl-6-[6-[methyl(spiro[2.3]hexan-5-yl)amino]-2-pyridyl]-3,4-dihydro-2H-pyridine-1-carboxylate

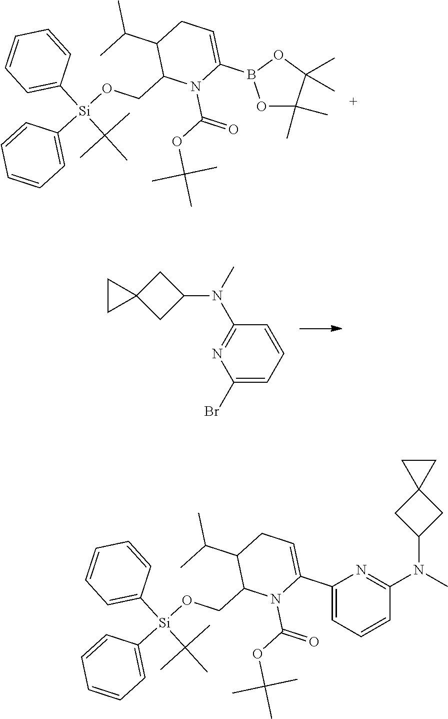 [SMILES: CC(C)C1CC=C(B2OC(C)(C)C(C)(C)O2)N(C(=O)OC(C)(C)C)C1CO[Si](c1ccccc1)(c1ccccc1)C(C)(C)C ; CN(c1cccc(Br)n1)C1CC2(CC2)C1 ; CC(C)C1CC=C(c2cccc(N(C)C3CC4(CC4)C3)n2)N(C(=O)OC(C)(C)C)C1CO[Si](c1ccccc1)(c1ccccc1)C(C)(C)C]

To a thick walled pressure vessel was added tert-butyl 2-[[tert-butyl(diphenyl)silyl]oxymethyl]-3-isopropyl-6-(4,4,5,5-tetramethyl-1,3,2-dioxaborolan-2-yl)-3,4-dihydro-2H-pyridine-1-carboxylate (1.62 g, 2.614 mmol), 6-bromo-N-methyl-N-spiro[2.3]hexan-5-yl-pyridin-2-amine (838 mg, 3.137 mmol), cesium; hydroxide; hydrate (878 mg, 5.228 mmol), tris(4-fluorophenyl)phosphane (149 mg, 0.4711 mmol) and Palladium (II) acetate (53 mg, 0.2361 mmol). The vial was sealed with a septum and placed under a nitrogen atmosphere. Toluene (16 mL) was added to the reaction mixture and the mixture was degassed by bubbling nitrogen through the solution for 10 minutes. The septum was removed, the reactor was flushed with argon and sealed with a Teflon screw cap. The reaction mixture was heated at 100° C. for 2 hours. The reaction mixture was cooled to room temperature and filtered through a pad of celite. The filtrate was concentrated to an oil under reduced pressure and then purified by silica gel column chromatography using a gradient of 0-8% EtOAc in hexanes to provide tert-butyl 2-[[tert-butyl(diphenyl)silyl]oxymethyl]-3-isopropyl-6-[6-[methyl(spiro[2.3]hexan-5-yl)amino]-2-pyridyl]-3,4-dihydro-2H-pyridine-1-carboxylate (1.64 g, 92%) as a sticky white foam. ¹H NMR (400 MHz, DMSO-d₆) δ 7.73-7.54 (m, 4H), 7.51-7.27 (m, 7H), 6.68 (d, J=7.4 Hz, 1H), 6.47 (d, J=8.4 Hz, 1H), 5.87 (d, J=107.9 Hz, 1H), 4.93 (p, J=8.1 Hz, 1H), 4.49 (s, 1H), 3.70 (s, 1H), 3.45 (s, 1H), 2.90 (s, 3H), 2.33 (dt, J=20.0, 10.2 Hz, 2H), 2.10 (dd, J=22.7, 10.2 Hz, 3H), 1.88 (d, J=17.4 Hz, 1H), 1.55 (d, J=7.8 Hz, 3H), 1.43-1.09 (m, 3H), 1.02 (d, J=3.3 Hz, 15H), 0.90-0.81 (m, 6H), 0.46-0.37 (m, 3H). ESI-MS m/z calc. 679.41693, found 680.6 (M+1)⁺; Retention time: 0.52 minutes; LC method D (50-99% gradient).

### Step 3: 6-[6-[[tert-Butyl(diphenyl)silyl]oxymethyl]-5-isopropyl-2-piperidyl]-N-methyl-N-spiro[2.3]hexan-5-yl-pyridin-2-amine

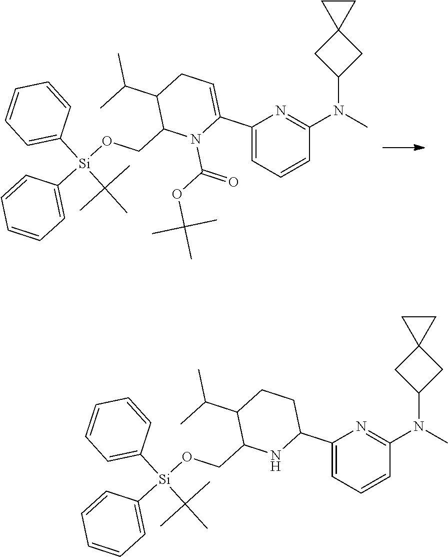 [SMILES: CC(C)C1CC=C(c2cccc(N(C)C3CC4(CC4)C3)n2)N(C(=O)OC(C)(C)C)C1CO[Si](c1ccccc1)(c1ccccc1)C(C)(C)C ; CC(C)C1CCC(c2cccc(N(C)C3CC4(CC4)C3)n2)NC1CO[Si](c1ccccc1)(c1ccccc1)C(C)(C)C]

tert-Butyl 2-[[tert-butyl(diphenyl)silyl]oxymethyl]-3-isopropyl-6-[6-[methyl(spiro[2.3]hexan-5-yl)amino]-2-pyridyl]-3,4-dihydro-2H-pyridine-1-carboxylate (800 mg, 1.176 mmol) was dissolved in DCM (11.2 mL) and cooled in an ice water bath. TFA (3.6 mL, 46.73 mmol) was added to the reaction, the cooling bath was removed after 5 minutes and the reaction was warmed to room temperature and continued to stir at this temperature for 1 hour. The reaction was then concentrated under reduced pressure to provide intermediate 6-[2-[[tert-butyl(diphenyl)silyl]oxymethyl]-3-isopropyl-1,2,3,4-tetrahydropyridin-6-yl]-N-methyl-N-spiro[2.3]hexan-5-yl-pyridin-2-amine (Trifluoroacetate salt) which was then dissolved in THE (21.6 mL) followed by the addition of triethylamine (328 μL, 2.353 mmol) and then sodium triacetoxyborohydride (997 mg, 4.704 mmol). The reaction mixture was stirred at room temperature for 16 hours. The reaction mixture was quenched with aqueous HCl (2 mL of 6 M, 12.00 mmol) and partitioned between ethyl acetate and saturated aqueous sodium chloride solution. The layers were separated, and the aqueous layer was extracted once more with ethyl acetate. The combined organics were dried over sodium sulfate, filtered and concentrated under reduced pressure. The crude material was purified by reverse phase HPLC using (10-99% ACN/water (5 mM HCl)). The desired factions were combined and partitioned between ethyl acetate and saturated aqueous sodium chloride solution. The layers were separated, and the aqueous layer was extracted once more with ethyl acetate. The combined organics were dried over sodium sulfate, filtered and concentrated under reduced pressure to provide 6-[6-[[tert-butyl(diphenyl)silyl]oxymethyl]-5-isopropyl-2-piperidyl]-N-methyl-N-spiro[2.3]hexan-5-yl-pyridin-2-amine (hydrochloride salt) (670 mg, 92%). ESI-MS m/z calc. 581.3801, found 582.9 (M+1)⁺; Retention time: 0.83 minutes; LC method D.

### Step 4: [3-Isopropyl-6-[6-[methyl(spiro[2.3]hexan-5-yl)amino]-2-pyridyl]-2-piperidyl]methanol

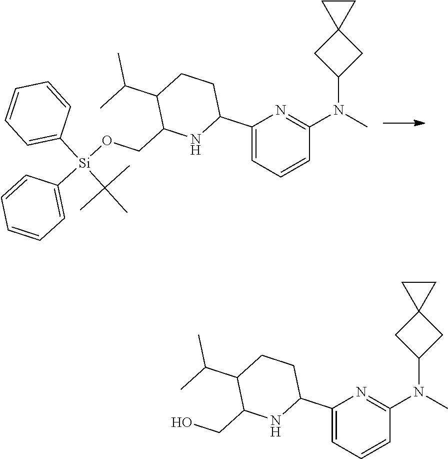

6-[6-[[tert-Butyl(diphenyl)silyl]oxymethyl]-5-isopropyl-2-piperidyl]-N-methyl-N-spiro[2.3]hexan-5-yl-pyridin-2-amine (hydrochloride salt) (530 mg, 0.8571 mmol) was dissolved in anhydrous THE (5.3 mL) under a nitrogen atmosphere. The reaction mixture was cooled in an ice water bath and then tetrabutylammonium fluoride (2.2 mL of 1 M, 2.200 mmol) as a solution in THF was added. The cooling bath was removed allowing the reaction mixture to warm to room temperature and continued to stir at this temperature for 20 hours. The solvent was removed under reduced pressure and the crude material was purified by silica gel column chromatography using a gradient of 0-8% MeOH in DCM to provide [3-isopropyl-6-[6-[methyl(spiro[2.3]hexan-5-yl)amino]-2-pyridyl]-2-piperidyl]methanol (288 mg, 98%). ESI-MS m/z calc. 343.26236, found 344.6 (M+1)⁺; Retention time: 0.53 minutes; LC method D.

### Step 5: 3-[[4-(2,6-Dimethylphenyl)-6-[[3-isopropyl-6-[6-[methyl(spiro[2.3]hexan-5-yl)amino]-2-pyridyl]-2-piperidyl]methoxy]pyrimidin-2-yl]sulfamoyl]benzoic acid

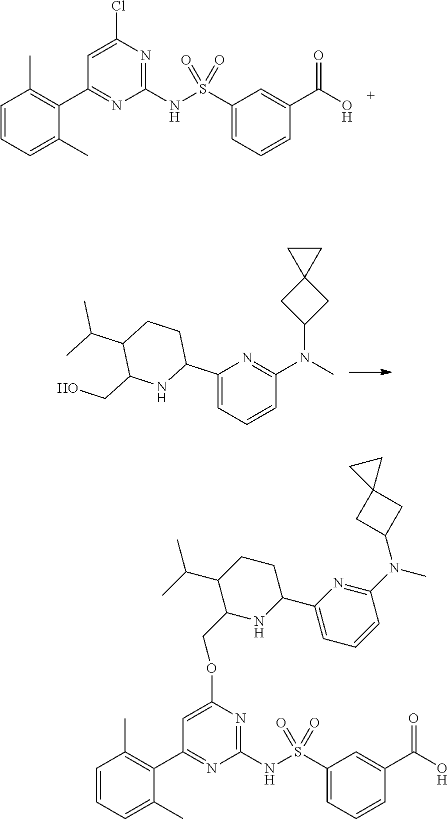 [SMILES: Cc1cccc(C)c1-c1cc(Cl)nc(NS(=O)(=O)c2cccc(C(=O)O)c2)n1 ; CC(C)C1CCC(c2cccc(N(C)C3CC4(CC4)C3)n2)NC1CO ; Cc1cccc(C)c1-c1cc(OCC2NC(c3cccc(N(C)C4CC5(CC5)C4)n3)CCC2C(C)C)nc(NS(=O)(=O)c2cccc(C(=O)O)c2)n1]

3-[[4-Chloro-6-(2,6-dimethylphenyl)pyrimidin-2-yl]sulfamoyl]benzoic acid (195 mg, 0.4667 mmol) and [3-isopropyl-6-[6-[methyl(spiro[2.3]hexan-5-yl)amino]-2-pyridyl]-2-piperidyl]methanol (160.3 mg, 0.4667 mmol) were combined in a flask and dissolved in THE (7.8 mL) followed by the addition of sodium tert-butoxide (269 mg, 2.799 mmol). The reaction mixture was stirred for 3 hours at room temperature. The reaction mixture was partitioned between DCM, 1 M aq. HCl and saturated aqueous sodium chloride solution. The layers were separated, and the organic layer was dried over sodium sulfate, filtered, and concentrated to a solid under reduced pressure. The crude material was purified by reverse phase HPLC using (1-60% ACN/water (5 mM HCl)). The desired fractions were combined and diluted with ethyl acetate and saturated aqueous sodium chloride solution. The layers were separated, and the aqueous layer was extracted once more with ethyl acetate. The combined organics were dried over sodium sulfate, filtered and concentrated under reduced pressure and further dried under high vacuum to provide 3-[[4-(2,6-dimethylphenyl)-6-[[3-isopropyl-6-[6-[methyl(spiro[2.3]hexan-5-yl)amino]-2-pyridyl]-2-piperidyl]methoxy]pyrimidin-2-yl]sulfamoyl]benzoic acid (hydrochloride salt) (220 mg, 62%) as a white solid. ESI-MS m/z calc. 724.3407, found 725.7 (M+1)⁺; Retention time: 0.64 minutes; LC method D.

### Step 6: 13-(2,6-dimethylphenyl)-7-isopropyl-4-[6-[methyl(spiro[2.3]hexan-5-yl)amino]-2-pyridyl]-17,17-dioxo-10-oxa-17λ⁶-thia-3,14,16,23-tetrazatetracyclo[16.3.1.111,15.03,8]tricosa-1(21),11,13,15(23),18(22),19-hexaen-2-one, isomer 1 (Compound 390) and 13-(2,6-dimethylphenyl)-7-isopropyl-4-[6-[methyl(spiro[2.3]hexan-5-yl)amino]-2-pyridyl]-17,17-dioxo-10-oxa-17λ⁶-thia-3,14,16,23-tetrazatetracyclo[16.3.1.111,15.03,8]tricosa-1(21),11,13,15(23),18(22),19-hexaen-2-one, isomer 2 (Compound 391)

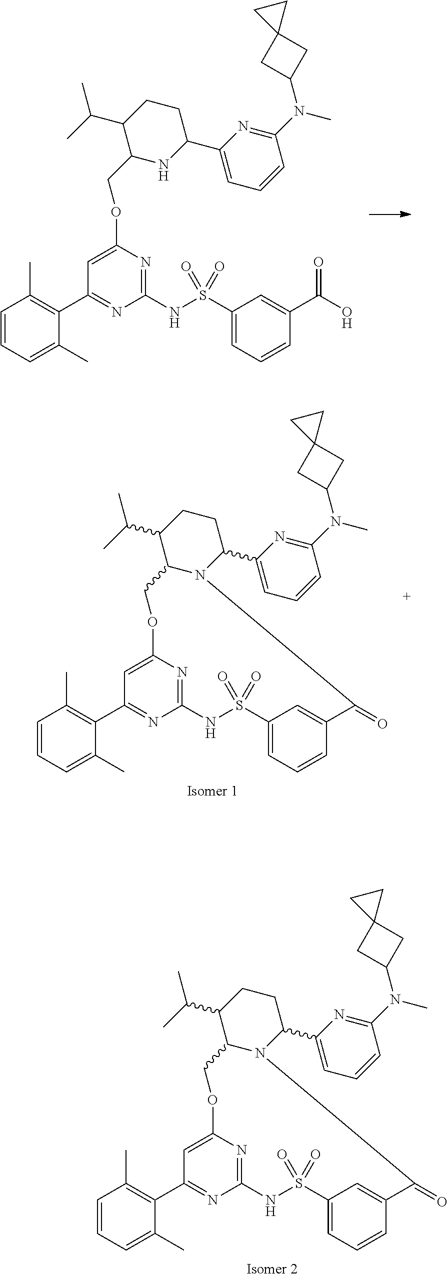 [SMILES: Cc1cccc(C)c1-c1cc(OCC2NC(c3cccc(N(C)C4CC5(CC5)C4)n3)CCC2C(C)C)nc(NS(=O)(=O)c2cccc(C(=O)O)c2)n1 ; Cc1cccc(C)c1-c1cc2nc(n1)NS(=O)(=O)c1cccc(c1)C(=O)N1C(c3cccc(N(C)C4CC5(CC5)C4)n3)CCC(C(C)C)C1CO2 ; Cc1cccc(C)c1-c1cc2nc(n1)NS(=O)(=O)c1cccc(c1)C(=O)N1C(c3cccc(N(C)C4CC5(CC5)C4)n3)CCC(C(C)C)C1CO2]

3-[[4-(2,6-dimethylphenyl)-6-[[3-isopropyl-6-[6-[methyl(spiro[2.3]hexan-5-yl)amino]-2-pyridyl]-2-piperidyl]methoxy]pyrimidin-2-yl]sulfamoyl]benzoic acid (hydrochloride salt) (220 mg, 0.2890 mmol) was dissolved in DMF (25 mL) and then cooled in an ice water bath. 2-chloro-4,6-dimethoxy-1,3,5-triazine (66 mg, 0.3759 mmol) was added to the reaction mixture followed by the addition of 4-methylmorpholine (205 mg, 2.027 mmol). After five minutes the cooling bath was removed allowing the reaction to warm to room temperature and continued to stir at this temperature for 12 hours. The reaction mixture was partitioned between EtOAc and 1 M aq. HCl. The layers were separated, and the organic layer was washed twice with saturated aqueous sodium chloride solution. The organic layer was dried over sodium sulfate, filtered, and concentrated under reduced pressure. The crude material was purified by reverse phase HPLC using (10-99% ACN/water (5 mM HCl)) to provide 13-(2,6-dimethylphenyl)-7-isopropyl-4-[6-[methyl(spiro[2.3]hexan-5-yl)amino]-2-pyridyl]-17,17-dioxo-10-oxa-17λ⁶-thia-3,14,16,23-tetrazatetracyclo[16.3.1.111,15.03,8]tricosa-1(21),11,13,15(23),18(22),19-hexaen-2-one as a racemic mixture (200 mg, 97%). ESI-MS m/z calc. 706.33014, found 707.8 (M+1)⁺; Retention time: 1.85 minutes. The enantiomers were separated by chiral SFC using a Phenomenex LUX-1 column (ChiralCel OD Equiv. 250×21.2 mm; 5 m) at 50° C. Mobile phase was 24% MeOH (w/20 mM NH₃), 76% CO₂ at a 70 mL/min flow. Concentration of the sample was ˜20.5 mg/mL in methanol (no modifier), injection volume 500 μL with an outlet pressure of 154 bar, detection wavelength of 210 nm to provide, isomer 1, Peak 1, 13-(2,6-dimethylphenyl)-7-isopropyl-4-[6-[methyl(spiro[2.3]hexan-5-yl)amino]-2-pyridyl]-17,17-dioxo-10-oxa-17λ⁶-thia-3,14,16,23-tetrazatetracyclo[16.3.1.111,15.03,8]tricosa-1(21),11,13,15(23),18(22),19-hexaen-2-one (57.7 mg, 56%) ESI-MS m/z calc. 706.33014, found 707.4 (M+1)⁺; Retention time: 1.84 minutes; and isomer 2, peak 2, 13-(2,6-dimethylphenyl)-7-isopropyl-4-[6-[methyl(spiro[2.3]hexan-5-yl)amino]-2-pyridyl]-17,17-dioxo-10-oxa-17λ⁶-thia-3,14,16,23-tetrazatetracyclo[16.3.1.111,15.03,8]tricosa-1(21),11,13,15(23),18(22),19-hexaen-2-one (69 mg, 67%) ESI-MS m/z calc. 706.33014, found 707.4 (M+1)⁺; Retention time: 1.84 minutes; LC method A.

### Example 147: Preparation of Compound 392 and Compound 393

### Step 1: 01-tert-Butyl 02-methyl 3-isobutylpiperidine-1,2-dicarboxylate

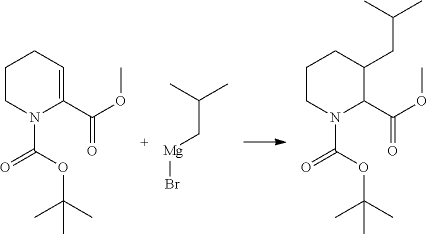

Isobutyl magnesium bromide (in THF) (380 mL of 1 M, 380.00 mmol) was added to copper(I) iodide (36 g, 189.03 mmol) in THE (140 mL) at −78° C. and then warmed up to −35° C. for 45 min. then cooled to −78° C. and 01-tert-butyl 06-methyl 3,4-dihydro-2H-pyridine-1,6-dicarboxylate (9.3 g, 38.544 mmol) was added dissolved in THE (140 mL). The reaction mixture was then left to warm-up to −10° C. and stirred at this temperature for 30 min. The reaction mixture was quenched with concentrated ammonium chloride (200 mL). It was left to warm-up, stirring at room temperature for 30 min. The aqueous phase was separated, then extracted with MeTHF (2×200 mL). The combined organic phases were dried with sodium sulfate, concentrated under reduced pressure and the resulting residue was purified on silica gel using 0% then 5% ethyl acetate in heptanes to provide 01-tert-butyl 02-methyl 3-isobutylpiperidine-1,2-dicarboxylate (7.2 g, 61%) as a clear oil ¹H NMR (400 MHz, CDCl₃) δ 4.82-4.48 (m, 1H), 4.18-3.86 (m, 1H), 3.74 (s, 3H), 3.09-2.79 (m, 1H), 2.34 (br. s., 1H), 1.69-1.57 (m, 2H), 1.50-1.34 (m, 13H), 1.26-1.19 (m, 1H), 0.93 (d, J=3.9 Hz, 3H), 0.91 (d, J=3.4 Hz, 3H). ESI-MS m/z calc. 299.2097, found 200.2 (M−99)⁺; Retention time: 2.71 minutes; LC method X.

### Step 2: tert-butyl 2-(hydroxymethyl)-3-isobutyl-piperidine-1-carboxylate

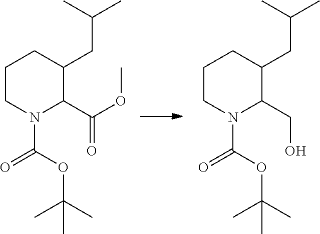 [SMILES: COC(=O)C1C(CC(C)C)CCCN1C(=O)OC(C)(C)C ; CC(C)CC1CCCN(C(=O)OC(C)(C)C)C1CO]

DIBAL (1M solution in tol.) (35 mL of 1 M, 35.000 mmol) was added dropwise to a solution of O1-tert-butyl 02-methyl 3-isobutylpiperidine-1,2-dicarboxylate (4.5 g, 15.030 mmol) in toluene (90 mL) maintained at 0° C. The reaction was kept at this temperature for 45 min then quenched with aqueous saturated Rochels salts solution (100 mL) at 0° C. and ethyl acetate (100 mL) was added and stirred a room temperature for 60 min until the phases could be separated. The aqueous phase was extracted with ethyl acetate (3×100 mL). The combined organic phases were dried over sodium sulfate, concentrated under reduced pressure and purified on silica gel to provide tert-butyl 2-(hydroxymethyl)-3-isobutyl-piperidine-1-carboxylate (3.9 g, 90%) as a clear oil. ¹H NMR (400 MHz, CDCl₃) δ 4.16-4.07 (m, 1H), 4.06-3.93 (m, 1H), 3.91-3.83 (m, 1H), 3.71-3.57 (m, 1H), 2.92-2.74 (m, 1H), 1.75-1.54 (m, 5H), 1.50-1.30 (m, 12H), 1.24-1.14 (m, 1H), 0.89 (d, J=6.6 Hz, 6H). ESI-MS m/z calc. 271.2147, found 294.2 (M+23)⁺; Retention time: 1.89 minutes; LC method X.

### Step 3: tert-Butyl 2-[[tert-butyl(diphenyl)silyl]oxymethyl]-3-isobutyl-piperidine-1-carboxylate

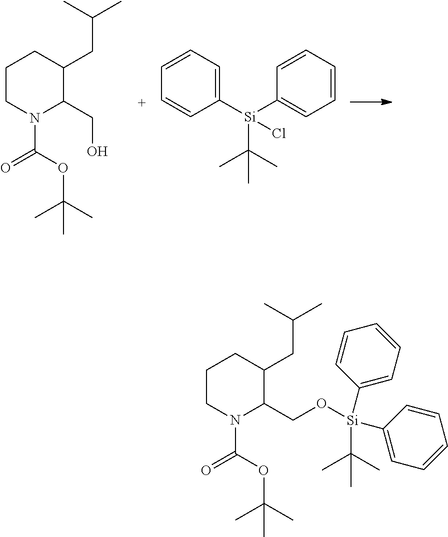 [SMILES: CC(C)CC1CCCN(C(=O)OC(C)(C)C)C1CO ; CC(C)(C)[Si](Cl)(c1ccccc1)c1ccccc1 ; CC(C)CC1CCCN(C(=O)OC(C)(C)C)C1CO[Si](c1ccccc1)(c1ccccc1)C(C)(C)C]

tert-Butyl-chloro-diphenyl-silane (1.2 g, 4.3658 mmol) was added to a solution of tert-butyl 2-(hydroxymethyl)-3-isobutyl-piperidine-1-carboxylate (800 mg, 2.9477 mmol) and imidazole (500 mg, 7.3446 mmol) in DCM (8 mL) at 0° C. The reaction mixture was left warm-up and stirred at room temperature for 1 h. The reaction was diluted with DCM (20 mL) and brine (10 mL). The aqueous layer was then extracted with DCM (2×20 ml). The combined organic solution was dried over anhydrous sodium sulfate, filtered and concentrated under vacuum to provide crude tert-butyl 2-[[tert-butyl(diphenyl)silyl]oxymethyl]-3-isobutyl-piperidine-1-carboxylate (1.4 g, 65%) as a yellow oil. This material was used in the next Step without further purification. An analytical sample was prepared by purification of a 100 mg crude sample by reverse phase chromatography on 30 g C₁₈ RediSep Rf gold column using a 5 the 60 to 100% gradient of acetonitrile in acidic water (0.1% formic acid content) to provide tert-butyl 2-[[tert-butyl(diphenyl)silyl]oxymethyl]-3-isobutyl-piperidine-1-carboxylate (71 mg, 5%) as a clear oil. ¹H NMR (400 MHz, CDCl₃) δ 7.76-7.63 (m, 4H), 7.49-7.36 (m, 6H), 4.34-4.11 (m, 1H), 4.09-3.80 (m, 1H), 3.78-3.68 (m, 2H), 2.72-2.52 (m, 1H), 1.97-1.85 (m, 1H), 1.76-1.64 (m, 1H), 1.62-1.53 (m, 2H), 1.45 (s, 9H), 1.44-1.26 (m, 3H), 1.17-1.08 (m, 1H), 1.06 (s, 9H), 0.97-0.85 (m, 6H). ESI-MS m/z calc. 509.3325, found 410.4 (M−99)⁺; Retention time: 3.09 minutes; LC method X.

### Step 4: tert-Butyl 2-[[tert-butyl(diphenyl)silyl]oxymethyl]-3-isobutyl-6-oxo-piperidine-1-carboxylate

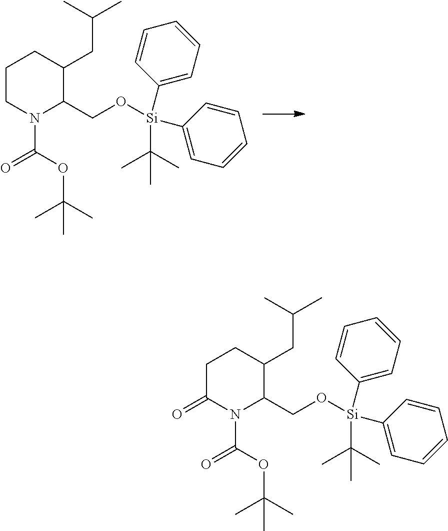 [SMILES: CC(C)CC1CCCN(C(=O)OC(C)(C)C)C1CO[Si](c1ccccc1)(c1ccccc1)C(C)(C)C ; CC(C)CC1CCC(=O)N(C(=O)OC(C)(C)C)C1CO[Si](c1ccccc1)(c1ccccc1)C(C)(C)C]

Sodium periodate (6.5 g, 30.389 mmol) and ruthenium(III) chloride hydrate (100 mg, 0.4436 mmol) were added to a solution of tert-butyl 2-[[tert-butyl(diphenyl)silyl]oxymethyl]-3-isobutyl-piperidine-1-carboxylate (1.4 g, 1.9223 mmol) in CCl4 (9 mL) and acetonitrile (9 mL) and water (11 mL) maintained at 0° C. for 15 min, then left to warm up to room temperature and stirred for 2 h. Ethyl acetate (50 mL) was added to the reaction mixture which was then filtered on silica gel. The filtrate was passed on a syringe filter, concentrated under reduced pressure and the resulting residue was purified by reverse phase chromatography on a C₁₈ column using a 5 then 70 to 100% gradient of acetonitrile in acidic water (0.1% formic acid content) to provide tert-butyl 2-[[tert-butyl(diphenyl)silyl]oxymethyl]-3-isobutyl-6-oxo-piperidine-1-carboxylate (537 mg, 53%) as a clear oil. ¹H NMR (400 MHz, CDCl₃) δ 7.69-7.60 (m, 4H), 7.48-7.34 (m, 6H), 4.04 (dt, J=6.5, 3.5 Hz, 1H), 3.75-3.68 (m, 1H), 3.67-3.61 (m, 1H), 2.59-2.47 (m, 1H), 2.45-2.34 (m, 1H), 2.29-2.21 (m, 1H), 2.00-1.90 (m, 1H), 1.72-1.61 (m, 1H), 1.51-1.40 (m, 10H), 1.35-1.27 (m, 2H), 1.07-1.01 (m, 9H), 0.96-0.90 (m, 6H). ESI-MS m/z calc. 523.3118, found 424.4 (M−99)⁺; Retention time: 2.55 minutes; LC method X.

### Step 5: tert-Butyl 2-[[tert-butyl(diphenyl)silyl]oxymethyl]-3-isobutyl-6-(trifluoromethylsulfonyloxy)-3,4-dihydro-2H-pyridine-1-carboxylate

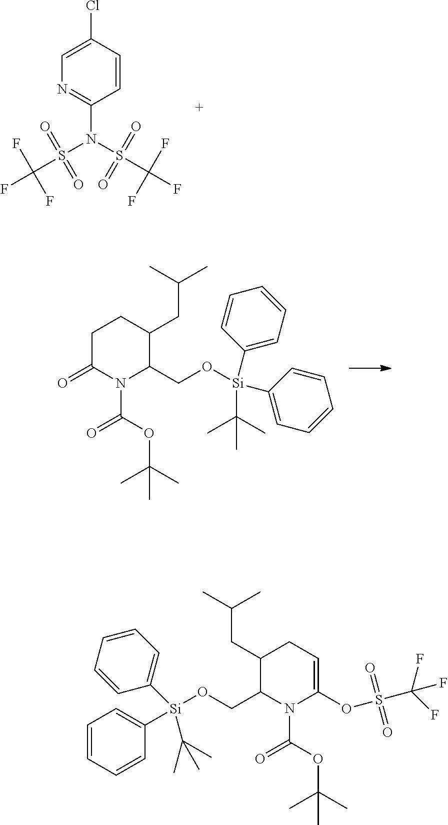 [SMILES: O=S(=O)(N(c1ccc(Cl)cn1)S(=O)(=O)C(F)(F)F)C(F)(F)F ; CC(C)CC1CCC(=O)N(C(=O)OC(C)(C)C)C1CO[Si](c1ccccc1)(c1ccccc1)C(C)(C)C ; CC(C)CC1CC=C(OS(=O)(=O)C(F)(F)F)N(C(=O)OC(C)(C)C)C1CO[Si](c1ccccc1)(c1ccccc1)C(C)(C)C]

In a 3 neck flask equipped with a magnetic stirrer and an internal temperature sensor, a solution of tert-butyl 2-[[tert-butyl(diphenyl)silyl]oxymethyl]-3-isobutyl-6-oxo-piperidine-1-carboxylate (500 mg, 0.9536 mmol) in anhydrous THF (10 mL) was cooled at −78° C. under nitrogen atmosphere. Lithium bis(trimethylsilyl)amide (1M in THF) (1.7 mL of 1 M, 1.7000 mmol) was added drop wise at −78° C. The reaction was stirred at the same temperature for 30 min. A solution of N-(5-chloro-2-pyridyl)-1,1,1-trifluoro-N-(trifluoromethylsulfonyl) methanesulfonamide (530 mg, 1.3497 mmol) in anhydrous THF (1 mL) was added to the reaction mixture drop wise. The reaction was then stirred at −78° C. for 1 hour. It was warmed up to −40° C. and stirred for 15 min. The reaction was quenched with saturated sodium bicarbonate (150 mL) at −40° C. and the temperature was then raised to rt. Water (300 mL) was added to the mixture and the aqueous phase was extracted with MTBE (3×150 mL). The combined organic layers were washed with brine (2×50 mL), dried over anhydrous Na₂SO₄, filtered and concentrated under vacuum. The resulting residue was purified on silica gel using 0 to 20% ethyl acetate in heptane to provide tert-butyl 2-[[tert-butyl(diphenyl)silyl]oxymethyl]-3-isobutyl-6-(trifluoromethylsulfonyloxy)-3,4-dihydro-2H-pyridine-1-carboxylate (550 mg, 87%) as a clear oil. ¹H NMR (400 MHz, CDCl₃) δ 7.71-7.60 (m, 4H), 7.50-7.33 (m, 6H), 5.04 (t, J=3.9 Hz, 1H), 4.52-4.41 (m, 1H), 3.81 (dd, J=10.3, 6.6 Hz, 1H), 3.52 (dd, J=10.3, 8.3 Hz, 1H), 2.20-2.11 (m, 1H), 2.10-2.02 (m, 1H), 1.83 (dd, J=18.6, 4.2 Hz, 1H), 1.74-1.60 (m, 1H), 1.49 (s, 9H), 1.16 (ddd, J=14.0, 7.6, 6.7 Hz, 1H), 1.08-0.97 (m, 10H), 0.88 (d, J=6.6 Hz, 6H). 19F NMR (377 MHz, CDCl₃) δ −74.17 (s, 3F). ESI-MS m/z calc. 655.2611, not found; Retention time: 3.02 minutes; LC method Y.

### Step 6: tert-Butyl 2-[[tert-butyl(diphenyl)silyl]oxymethyl]-3-isobutyl-6-(4,4,5,5-tetramethyl-1,3,2-dioxaborolan-2-yl)-3,4-dihydro-2H-pyridine-1-carboxylate

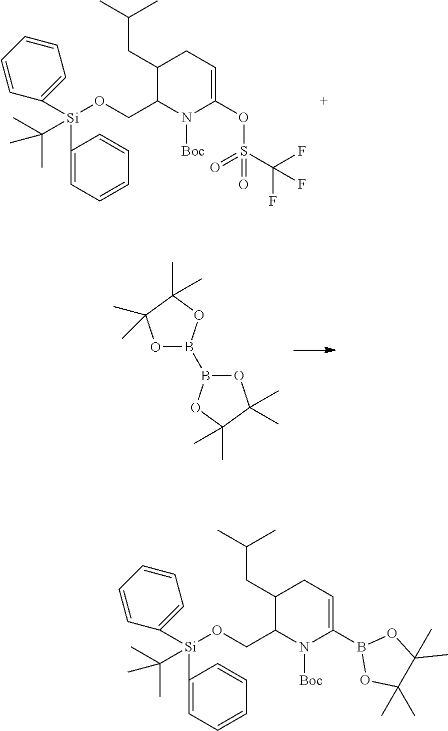

A 10 mL flask was charged with 4,4,5,5-tetramethyl-2-(4,4,5,5-tetramethyl-1,3,2-dioxaborolan-2-yl)-1,3,2-dioxaborolane (1 g, 3.9380 mmol), tert-butyl 2-[[tert-butyl(diphenyl)silyl]oxymethyl]-3-isobutyl-6-(trifluoromethylsulfonyloxy)-3,4-dihydro-2H-pyridine-1-carboxylate (1.7 g, 2.4625 mmol), potassium carbonate (920 mg, 6.6567 mmol), Pd(PPh3)₂Cl₂ (189 mg, 0.2693 mmol), triphenylphosphine (141 mg, 0.5376 mmol) under argon atmosphere. A pre-degassed 1,4-dioxane (30 mL) (bubbling argon for 2 h with stirring) was added to the reaction flask. The reaction mixture was further degassed with argon for 50 minutes. The reaction was then heated at 90° C. for 1 hours 30 min. The reaction was cooled to room temperature. The reaction was diluted with ethyl acetate (50 mL) and filtered through a pad of celite washing thoroughly with EtOAc. Brine (50 ml) was added to the filtrate. Two layers were separated, and the aqueous layer was extracted with ethyl acetate (2×50 mL). The combined organic layers were dried over anhydrous sodium sulfate and concentrated under vacuum. The crude product was purified by flash chromatography (Combiflash, loaded with minimum amount of benzene in 40 g cartridge using 0-15% diethyl ether in hexanes as eluent) and the solvent was removed to provide tert-butyl 2-[[tert-butyl(diphenyl)silyl]oxymethyl]-3-isobutyl-6-(4,4,5,5-tetramethyl-1,3,2-dioxaborolan-2-yl)-3,4-dihydro-2H-pyridine-1-carboxylate (1.1 g, 67%) as a colorless gel. ¹H NMR (500 MHz, Chloroform-d) δ 7.66-7.58 (m, 4H), 7.42-7.33 (m, 6H), 5.00 (d, J=5.5 Hz, 1H), 3.92 (t, J=7.6 Hz, 1H), 3.61 (dd, J=9.7, 5.3 Hz, 1H), 3.52 (t, J=9.8 Hz, 1H), 2.37 (d, J=7.1 Hz, 1H), 2.21-2.03 (m, 1H), 1.73 (dd, J=18.2, 5.8 Hz, 1H), 1.66-1.57 (m, 1H), 1.41 (s, 9H), 1.24 (d, J=4.4 Hz, 12H), 1.18-1.12 (m, 2H), 1.04 (s, 9H), 0.97-0.82 (m, 6H). ESI-MS m/z calc. 633.4021, found 634.6 (M+1)⁺; Retention time: 4.54 minutes; LC method W.

### Step 7: tert-Butyl 2-[[tert-butyl(diphenyl)silyl]oxymethyl]-3-isobutyl-6-(5-isopropoxypyrimidin-2-yl)-3,4-dihydro-2H-pyridine-1-carboxylate

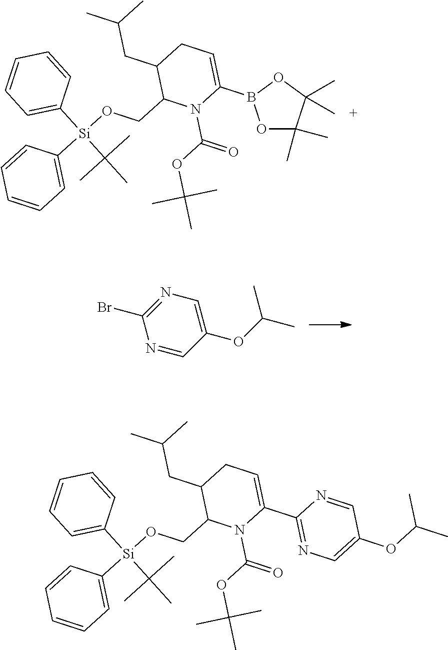 [SMILES: CC(C)CC1CC=C(B2OC(C)(C)C(C)(C)O2)N(C(=O)OC(C)(C)C)C1CO[Si](c1ccccc1)(c1ccccc1)C(C)(C)C ; CC(C)Oc1cnc(Br)nc1 ; CC(C)CC1CC=C(c2ncc(OC(C)C)cn2)N(C(=O)OC(C)(C)C)C1CO[Si](c1ccccc1)(c1ccccc1)C(C)(C)C]

To a thick walled pressure vessel was added tert-butyl 2-[[tert-butyl(diphenyl)silyl]oxymethyl]-3-isobutyl-6-(4,4,5,5-tetramethyl-1,3,2-dioxaborolan-2-yl)-3,4-dihydro-2H-pyridine-1-carboxylate (1 g, 1.578 mmol), 2-bromo-5-isopropoxy-pyrimidine (411.1 mg, 1.894 mmol), cesium; hydroxide; hydrate (530 mg, 3.156 mmol), tris(4-fluorophenyl)phosphane (99.81 mg, 0.3156 mmol) and diacetoxypalladium (35.4 mg, 0.1577 mmol). The vial was sealed with a septum and placed under a nitrogen atmosphere. Toluene (10 mL) was added to the reaction mixture and the mixture was degassed by bubbling nitrogen through the solution for 10 minutes. The septum was removed, the reactor was flushed with argon and sealed with a Teflon screw cap. The reaction mixture was heated at 100° C. for 2 hours. UPLC/MS analysis of the reaction mixture showed approximately 20% desired product formation and staring materials remaining. The reaction was charged with an additional tris(4-fluorophenyl)phosphane (99.81 mg, 0.3156 mmol) and diacetoxypalladium (35.4 mg, 0.1577 mmol) and continued to stir at 100° C. for an additional 2 hours. The reaction mixture was cooled to room temperature and filtered through a pad of celite. The filtrate was concentrated to an oil under reduced pressure and then purified by silica gel column chromatography using a gradient of 0-20% EtOAc in hexanes to provide tert-butyl 2-[[tert-butyl(diphenyl)silyl]oxymethyl]-3-isobutyl-6-(5-isopropoxypyrimidin-2-yl)-3,4-dihydro-2H-pyridine-1-carboxylate (505 mg, 50%) as a white solid. ESI-MS m/z calc. 643.38055, found 644.71 (M+1)⁺; Retention time: 0.87 minutes; LC method d (50-99% gradient).

### Step 8: tert-Butyl-[[3-isobutyl-6-(5-isopropoxypyrimidin-2-yl)-2-piperidyl]methoxy]-diphenyl-silane

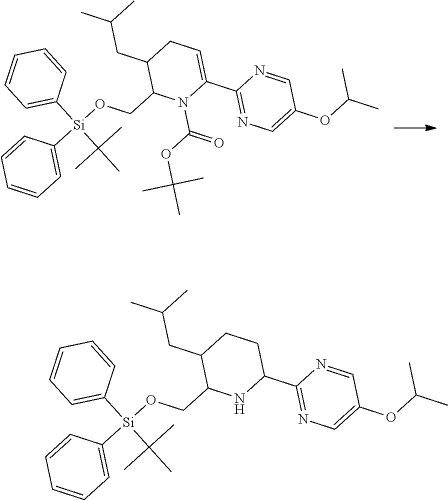 [SMILES: CC(C)CC1CC=C(c2ncc(OC(C)C)cn2)N(C(=O)OC(C)(C)C)C1CO[Si](c1ccccc1)(c1ccccc1)C(C)(C)C ; CC(C)CC1CCC(c2ncc(OC(C)C)cn2)NC1CO[Si](c1ccccc1)(c1ccccc1)C(C)(C)C]

tert-Butyl 2-[[tert-butyl(diphenyl)silyl]oxymethyl]-3-isobutyl-6-(5-isopropoxypyrimidin-2-yl)-3,4-dihydro-2H-pyridine-1-carboxylate (505 mg, 0.7842 mmol) was dissolved in DCM (7 mL) and cooled in an ice water bath. TFA (2.4 mL, 31.15 mmol) was added to the reaction, the cooling bath was removed after 5 minutes and the reaction was warmed to room temperature and continued to stir at this temperature for 2 hours. The reaction was then concentrated under reduced pressure to provide intermediate tert-butyl-[[3-isobutyl-6-(5-isopropoxypyrimidin-2-yl)-1,2,3,4-tetrahydropyridin-2-yl]methoxy]-diphenyl-silane (Trifluoroacetate salt) which was then dissolved in THE (14 mL) followed by the addition of triethylamine (220 μL, 1.578 mmol) and then sodium triacetoxyborohydride (665 mg, 3.138 mmol). The reaction mixture was stirred at room temperature for 16 hours. The reaction mixture was quenched with aqueous HCl (1.3 mL of 6 M, 7.800 mmol) and partitioned between ethyl acetate and saturated aqueous sodium chloride solution. The layers were separated, and the aqueous layer was extracted once more with ethyl acetate. The combined organics were dried over sodium sulfate, filtered, and concentrated under reduced pressure. The crude material was purified by reverse phase HPLC using (1-60% ACN/water (5 mM HCl)). The desired factions were combined and partitioned between ethyl acetate and saturated aqueous sodium chloride solution. The layers were separated, and the aqueous layer was extracted once more with ethyl acetate. The combined organics were dried over sodium sulfate, filtered and concentrated under reduced pressure to provide tert-butyl-[[3-isobutyl-6-(5-isopropoxypyrimidin-2-yl)-2-piperidyl]methoxy]-diphenyl-silane (hydrochloride salt) (300 mg, 66%). ESI-MS m/z calc. 545.34375, found 546.88 (M+1)⁺; Retention time: 0.75 minutes; LC method D.

### Step 9: [3-Isobutyl-6-(5-isopropoxypyrimidin-2-yl)-2-piperidyl]methanol

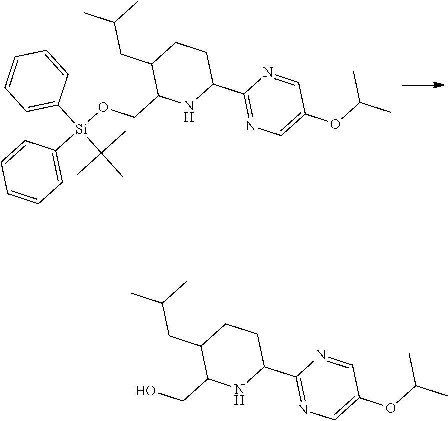

tert-Butyl-[[3-isobutyl-6-(5-isopropoxypyrimidin-2-yl)-2-piperidyl]methoxy]-diphenyl-silane (hydrochloride salt) (307.4 mg, 0.5279 mmol) was dissolved in dioxane (5 mL) followed by the addition of aqueous HCl (2.2 mL of 12 M, 26.40 mmol). The reaction mixture was heated at 100° C. for 1 hour. The reaction mixture was cooled to room temperature and the dioxane was removed under reduced pressure. The crude product was dissolved in 1 mL of methanol and 1 mL of DMSO and purified by reverse phase HPLC using a gradient of (1-40% ACN/water (5 mM HCl)). The desired fractions were combined and concentrated under reduced pressure and further dried under high vacuum to provide [3-isobutyl-6-(5-isopropoxypyrimidin-2-yl)-2-piperidyl]methanol (hydrochloride salt) (140 mg, 77%). ESI-MS m/z calc. 307.22598, found 308.6 (M+1)⁺; Retention time: 1.34 minutes; LC method I.

### Step 10: 3-[[4-(2,6-Dimethylphenyl)-6-[[3-isobutyl-6-(5-isopropoxypyrimidin-2-yl)-2-piperidyl]methoxy]pyrimidin-2-yl]sulfamoyl]benzoic acid

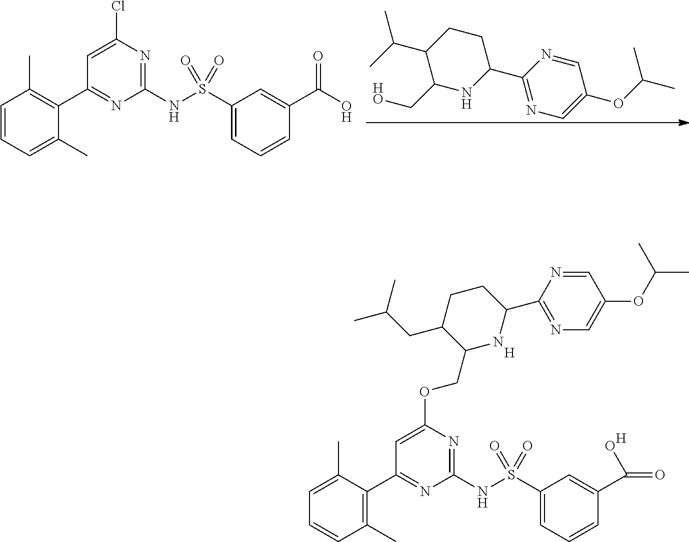

3-[[4-Chloro-6-(2,6-dimethylphenyl)pyrimidin-2-yl]sulfamoyl]benzoic acid (170 mg, 0.4068 mmol) and [3-isobutyl-6-(5-isopropoxypyrimidin-2-yl)-2-piperidyl]methanol (hydrochloride salt) (139.9 mg, 0.4068 mmol) were combined in a flask and dissolved in THF (6.8 mL) under a nitrogen atmosphere. sodium tert-butoxide (274 mg, 2.851 mmol) was added to the reaction mixture and continued to stir for 1 hour 20 minutes. The reaction mixture was cooled in and ice water bath and quenched with aqueous solution of HCl (700 μL of 6 M, 4.200 mmol). The reaction mixture was partitioned between ethyl acetate and saturated aqueous sodium chloride solution. The layers were separated, and the aqueous phase was extracted once more with ethyl acetate. The combined organics were dried over sodium sulfate, filtered and concentrated to dryness under reduced pressure. The crude material was purified by reverse phase HPLC using a gradient of (1-60% ACN/water (5 mM HCl)). The desired fractions were combined and the acetonitrile was removed under reduced pressure. The resulting aqueous solution was partitioned between ethyl acetate and saturated aqueous sodium chloride solution. The layers were separated, and the aqueous phase was extracted once more with ethyl acetate. The combined organics were dried over sodium sulfate, filtered and concentrated to dryness under reduced pressure and further dried under high vacuum to provide 3-[[4-(2,6-dimethylphenyl)-6-[[3-isobutyl-6-(5-isopropoxypyrimidin-2-yl)-2-piperidyl]methoxy]pyrimidin-2-yl]sulfamoyl]benzoic acid (hydrochloride salt) (155 mg, 53%) as a semi solid. ESI-MS m/z calc. 688.3043, found 689.8 (M+1)⁺; Retention time: 0.57 minutes; LC method D.

### Step 11: 13-(2,6-dimethylphenyl)-7-isobutyl-4-(5-isopropoxypyrimidin-2-yl)-17,17-dioxo-10-oxa-17λ⁶-thia-3,14,16,23-tetrazatetracyclo[16.3.1.111,15.03,8]tricosa-1(21),11,13,15(23),18(22),19-hexaen-2-one, isomer 1 (Compound 393), and 13-(2,6-dimethylphenyl)-7-isobutyl-4-(5-isopropoxypyrimidin-2-yl)-17,17-dioxo-10-oxa-17λ⁶-thia-3,14,16,23-tetrazatetracyclo[16.3.1.111,15.03,8]tricosa-1(21),11,13,15(23),18(22),19-hexaen-2-one, isomer 2 (Compound 392)

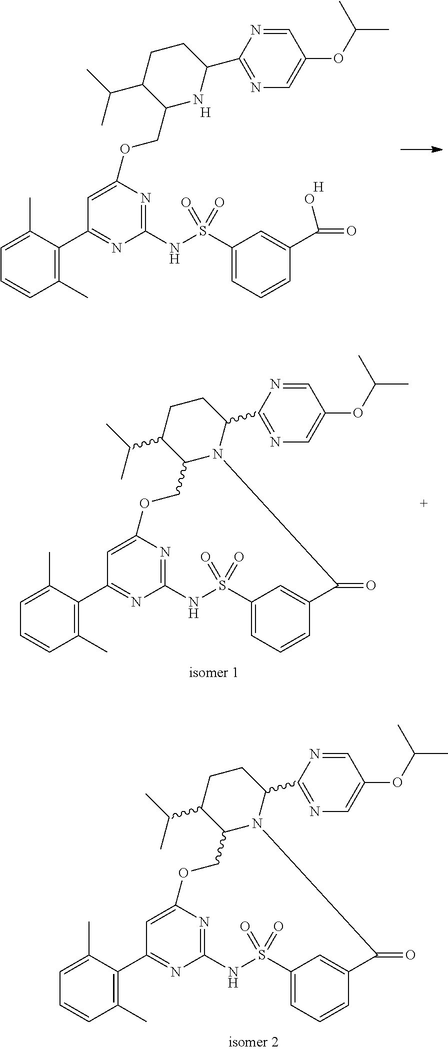

3-[[4-(2,6-Dimethylphenyl)-6-[[3-isobutyl-6-(5-isopropoxypyrimidin-2-yl)-2-piperidyl]methoxy]pyrimidin-2-yl]sulfamoyl]benzoic acid (hydrochloride salt) (155 mg, 0.2137 mmol) was dissolved in DMF (20 mL) and then cooled in an ice water bath. 2-chloro-4,6-dimethoxy-1,3,5-triazine (48.8 mg, 0.2779 mmol) was added to the reaction mixture followed by the addition of 4-methylmorpholine (165 μL, 1.501 mmol). After five minutes the cooling bath was removed allowing the reaction to warm to room temperature and continued to stir at this temperature for 12 hours. The reaction mixture was partitioned between EtOAc and 1 M aqueous HCl. The layers were separated, and the organic layer was washed twice with saturated aqueous sodium chloride solution. The organic layer was dried over sodium sulfate, filtered and concentrated under reduced pressure. The crude material was purified by reverse phase HPLC using a gradient of (10-99% ACN/water (5 mM HCl)). The desired fractions were combined and concentrated under reduced pressure. The product was precipitated with DCM/hexanes to provide 13-(2,6-dimethylphenyl)-7-isobutyl-4-(5-isopropoxypyrimidin-2-yl)-17,17-dioxo-10-oxa-17λ⁶-thia-3,14,16,23-tetrazatetracyclo[16.3.1.111,15.03,8]tricosa-1(21),11,13,15(23),18(22),19-hexaen-2-one (118 mg, 81%) as a white solid. ESI-MS m/z calc. 670.29376, found 671.3 (M+1)⁺; Retention time: 2.06 minutes. The diastereomers were separated by chiral SFC using a ChiralPak IG column (250×21.2 mm; 5 m) at 50° C. Mobile phase was 26% MeOH (20 mM NH₃), 74% CO₂ at a 70 mL/min flow. Concentration of the sample was 32.0 mg/mL in methanol (No Mod), injection volume 500 μL with an outlet pressure of 151 bar, detection wavelength of 210 nm to give isomer 1, SFC Peak 1, 13-(2,6-dimethylphenyl)-7-isobutyl-4-(5-isopropoxypyrimidin-2-yl)-17,17-dioxo-10-oxa-17λ⁶-thia-3,14,16,23-tetrazatetracyclo[16.3.1.111,15.03,8]tricosa-1(21),11,13,15(23),18(22),19-hexaen-2-one (28.5 mg, 39%), ¹H NMR (400 MHz, Chloroform-d) δ 8.70 (s, 1H), 8.42 (s, 2H), 7.71 (d, J=106.9 Hz, 2H), 7.07 (s, 1H), 6.91 (s, 2H), 6.16 (s, 1H), 5.90 (s, 1H), 5.14 (d, J=10.7 Hz, 1H), 4.65 (p, J=6.0 Hz, 1H), 3.68 (d, J=11.4 Hz, 1H), 3.50-3.30 (m, 1H), 2.54 (d, J=11.6 Hz, 1H), 1.99 (d, J=126.4 Hz, 11H), 1.40 (t, J=5.8 Hz, 7H), 1.03 (t, J=7.0 Hz, 1H), 0.93-0.82 (m, 1H), 0.67 (d, J=6.4 Hz, 3H), 0.57 (d, J=6.3 Hz, 3H). ESI-MS m/z calc. 670.29376, found 671.3 (M+1)⁺; Retention time: 2.05 minutes; and isomer 2, SFC Peak 2: 13-(2,6-dimethylphenyl)-7-isobutyl-4-(5-isopropoxypyrimidin-2-yl)-17,17-dioxo-10-oxa-17λ⁶-thia-3,14,16,23-tetrazatetracyclo[16.3.1.111,15.03,8]tricosa-1(21),11,13,15(23),18(22),19-hexaen-2-one (29.7 mg, 41%). ¹H NMR (400 MHz, Chloroform-d) δ 8.70 (s, 1H), 8.43 (s, 2H), 7.68 (d, J=98.5 Hz, 2H), 7.07 (s, 1H), 6.91 (s, 2H), 6.15 (d, J=5.9 Hz, 1H), 5.89 (s, 1H), 5.14 (d, J=10.6 Hz, 1H), 4.66 (h, J 6.0 Hz, 1H), 3.68 (d, J=11.3 Hz, 1H), 3.40 (d, J=6.5 Hz, 1H), 2.54 (d, J=11.4 Hz, 1H), 2.26-1.72 (m, 11H), 1.50-1.32 (m, 7H), 1.03 (dt, J=13.4, 6.6 Hz, 1H), 0.94-0.83 (m, 1H), 0.69 (d, J=6.3 Hz, 3H), 0.58 (d, J=6.3 Hz, 3H). ESI-MS m/z calc. 670.29376, found 671.3 (M+1)⁺; Retention time: 2.06 minutes; LC method A.

### Example 148: Preparation of Compound 394 and Compound 395

### Step 1: Ethyl 6-oxocyclohexene-1-carboxylate

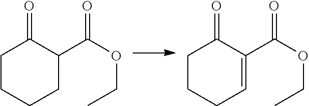

Phenylselenyl chloride (15 g, 78.322 mmol) was dissolved in DCM (400 mL). It is cooled to 0° C. and treated with pyridine (7.2372 g, 7.4 mL, 91.494 mmol) for 15 minutes. A solution of ethyl 2-oxocyclohexanecarboxylate (13 g, 76.378 mmol) in DCM (40 mL) was introduced. The reaction was stirred at 0° C. for 1.5 hours and allowed to warm to rt over 45 minutes. The solution was washed with 10% HCl (300 mL), saturated sodium bicarbonate (300 mL), and dried over anhydrous sodium sulfate. Evaporation of the solvent gave an orange oil. A round bottom flask was charged with the intermediate and DCM (200 mL). The solution was stirred at rt and a few drops of hydrogen peroxide was added to initiate the reaction. The solution was cooled to 0° C., and stirred vigorously while hydrogen peroxide (5.3280 g, 16 mL of 30% w/w, 46.991 mmol) was added dropwise over 20 minutes. After the addition, the reaction was stirred at rt for 15 minutes and at 0° C. for 15 minutes. The solid was filtered off and washed with DCM (50 mL). The combined filtrate was washed with 7% sodium bicarbonate (100 mL), dried over anhydrous sodium sulfate and concentrated under vacuum to furnish ethyl 6-oxocyclohexene-1-carboxylate (12.7 g, 94%) as an orange liquid. The crude material was used in the next step without purification. ¹H NMR (500 MHz, Chloroform-d) δ 7.65 (s, 1H), 4.26 (d, J=7.1 Hz, 2H), 2.65-2.40 (m, 4H), 2.15-1.92 (m, 2H), 1.31 (t, J=7.1 Hz, 3H).

### Step 2: Ethyl 2-isobutyl-6-oxo-cyclohexanecarboxylate

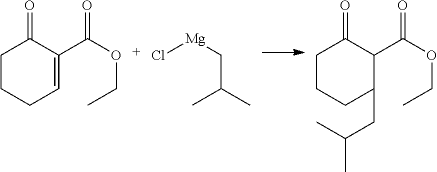

Into a suspension of CuI (38 g, 199.53 mmol) in anhydrous THE (67.000 mL) was added chloro(isobutyl)magnesium (2 M in THF) (200 mL of 2 M, 400.00 mmol) at −78° C. The reaction was stirred at −35° C. for 1 hour. The reaction was cooled to −78° C. A solution of ethyl 6-oxocyclohexene-1-carboxylate (6.7 g, 39.836 mmol) in anhydrous THE (67.000 mL) was added to the reaction mixture dropwise. The reaction was stirred at the same temperature for 30 minutes, then it was slowly warmed to 0° C. The reaction was quenched with sat ammonium chloride (50 mL) and diluted with ethyl acetate (300 mL). The solid was filtered off through a pad of Celite before two layers partitioned. The aqueous layer was extracted with ethyl acetate (2×300 mL). The combined organic layers were washed with brine (200 mL), dried over anhydrous sodium sulfate and concentrated under vacuum. The residue was purified by silica gel chromatography using 0 to 40% ether in hexane to furnish ethyl 2-isobutyl-6-oxo-cyclohexanecarboxylate (6.1 g, 66%) as a light yellow liquid. ¹H NMR (500 MHz, Chloroform-d) δ 12.74-12.35 (m, 0.7H), 4.42 (ddtt, J=17.9, 10.8, 7.1, 3.4 Hz, 2H), 3.26 (dd, J=10.4, 2.8 Hz, 0.3H), 2.84-2.72 (m, 1H), 2.46-2.27 (m, 3H), 2.01-1.76 (m, 3H), 1.64 (d, J=2.6 Hz, 1H), 1.50 (ddt, J=10.5, 7.3, 3.0 Hz, 5H), 1.30-0.96 (m, 6H).

### Step 3: Ethyl 3-isobutyl-7-oxo-azepane-2-carboxylate

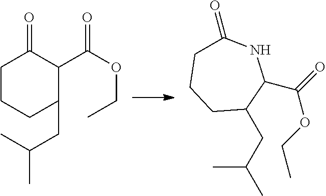 [SMILES: CCOC(=O)C1C(=O)CCCC1CC(C)C ; CCOC(=O)C1NC(=O)CCCC1CC(C)C]

Ethyl 2-isobutyl-6-oxo-cyclohexanecarboxylate (11 g, 48.605 mmol) was dissolved in chloroform (275 mL) and cooled to 0° C. Methanesulfonic acid (47.392 g, 32 mL, 493.12 mmol) was added followed by sodium azide (16 g, 246.12 mmol). The reaction was stirred at rt for 30 minutes. It was heated to reflux for 3 hours. Ice was added to the reaction mixture and stirred for 10 minutes; this was followed by addition of ammonium hydroxide until the reaction became basic. The reaction was extracted with DCM (3×100 mL). The combined organic layers were washed with brine (300 mL), and 2N Na₂CO₃ (300 mL), dried over anhydrous sodium sulfate and concentrated under vacuum. The residue was purified by silica gel chromatography using 0 to 35% acetone in hexane to furnish ethyl 3-isobutyl-7-oxo-azepane-2-carboxylate (11 g, 93%) as a yellow solid. ¹H NMR (500 MHz, Chloroform-d) δ 6.35-5.80 (m, 1H), 4.28 (dq, J=29.1, 7.1 Hz, 2H), 4.21-3.71 (m, 1H), 2.51-1.91 (m, 4H), 1.89-1.49 (m, 5H), 1.45-1.18 (m, 5H), 1.11-0.55 (m, 6H). ESI-MS m/z calc. 241.1678, found 242.3 (M+1)⁺; Retention time: 2.53 minutes; LC method T.

### Step 4: 7-(Hydroxymethyl)-6-isobutyl-azepan-2-one

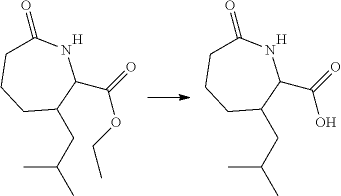

Into a solution of ethyl 3-isobutyl-7-oxo-azepane-2-carboxylate (2.21 g, 9.1577 mmol) in anhydrous DCM (25 mL) was added 2 M LiBH₄ in THE (5.1 mL of 2 M, 10.200 mmol) dropwise at 0° C. The reaction was stirred at rt for 16 hours. The reaction was quenched with 1N HCl (20 mL) at 0° C. The aqueous layer was extracted with ethyl acetate (5×20 mL). The combined organic layers were washed with brine (20 mL), dried over anhydrous magnesium sulfate and concentrated under vacuum to furnish 7-(hydroxymethyl)-6-isobutyl-azepan-2-one (1.76 g, 96%) as a clear gel. ¹H NMR (500 MHz, DMSO-d₆) δ 7.02-6.65 (m, 1H), 3.57-3.09 (m, 3H), 3.08-2.84 (m, 1H), 2.44-2.03 (m, 2H), 1.87-1.66 (m, 2H), 1.66-1.30 (m, 4H), 1.30-1.05 (m, 2H), 1.00-0.67 (m, 6H). ESI-MS m/z calc. 199.15723, found 200.2 (M+1)⁺; Retention time: 1.85 minutes; LC method T.

### Step 5: 7-[[tert-Butyl(dimethyl)silyl]oxymethyl]-6-isobutyl-azepan-2-one

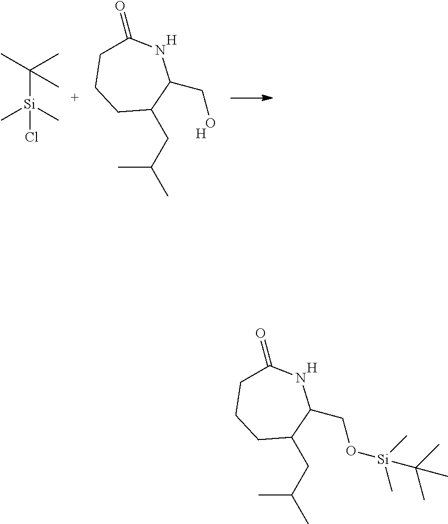

To a solution of 7-(hydroxymethyl)-6-isobutyl-azepan-2-one (5.1 g, 25.591 mmol) and tert-butyl-chloro-dimethyl-silane (11.6 g, 76.963 mmol) in anhydrous DMF (80 mL) was added imidazole (8.8 g, 129.26 mmol) at rt. The reaction was stirred for 3 h. The reaction was diluted with ethyl acetate (300 mL) and washed with brine (3×50 mL). The organic layer was dried over anhydrous sodium sulfate and concentrated under vacuum. The residue was purified by silica gel chromatography using 0 to 30% acetone in hexane to furnish 7-[[tert-butyl(dimethyl)silyl]oxymethyl]-6-isobutyl-azepan-2-one (6.19 g, 73%) as a clear liquid, which solidified upon standing. ¹H NMR (500 MHz, DMSO-d₆) δ 6.96-6.78 (m, 1H), 3.81-3.41 (m, 2H), 3.04-2.90 (m, 1H), 2.36-2.01 (m, 2H), 1.95-1.64 (m, 2H), 1.65-1.36 (m, 4H), 1.29-1.06 (m, 2H), 0.97-0.75 (m, 15H), 0.04 (t, J=4.2 Hz, 6H). ESI-MS m/z calc. 313.2437, found 314.0 (M+1)⁺; Retention time: 4.14 minutes; LC method T.

### Step 6: tert-Butyl 2-[[tert-butyl(dimethyl)silyl]oxymethyl]-3-isobutyl-7-oxo-azepane-1-carboxylate, isomer A, and tert-butyl 2-[[tert-butyl(dimethyl)silyl]oxymethyl]-3-isobutyl-7-oxo-azepane-1-carboxylate, isomer B

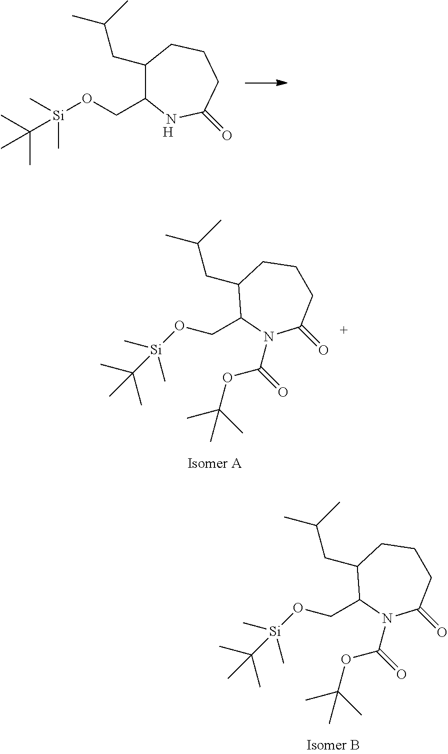

Into a solution of 7-[[tert-butyl(dimethyl)silyl]oxymethyl]-6-isobutyl-azepan-2-one (2.266 g, 7.2269 mmol) and Boc₂O (7.97 g, 8.3895 mL, 36.518 mmol) in anhydrous toluene (70 mL) was added DIEA (4.6746 g, 6.3 mL, 36.169 mmol) and DMAP (1.32 g, 10.805 mmol). The reaction was stirred at 110° C. for 2 hours. The volatiles were removed under vacuum. The residue was purified by silica gel chromatography (120 g column, Gradient: 0 to 10% ethyl acetate in hexane, 20 mL/min) to furnish isomer A (less polar) tert-butyl 2-[[tert-butyl(dimethyl)silyl]oxymethyl]-3-isobutyl-7-oxo-azepane-1-carboxylate (1.323 g, 44%) as a clear gel. ESI-MS m/z calc. 413.2961, found 414.4 (M+1)⁺; Retention time: 8.86 minutes and isomer B (more polar) tert-butyl 2-[[tert-butyl(dimethyl)silyl]oxymethyl]-3-isobutyl-7-oxo-azepane-1-carboxylate (0.875 g, 29%) as a clear gel. ESI-MS m/z calc. 413.2961, found 414.2 (M+1)⁺; Retention time: 8.75 minutes; LC method S.

### Step 7: tert-Butyl 2-[[tert-butyl(dimethyl)silyl]oxymethyl]-7-diphenylphosphoryloxy-3-isobutyl-2,3,4,5-tetrahydroazepine-1-carboxylate, isomer A

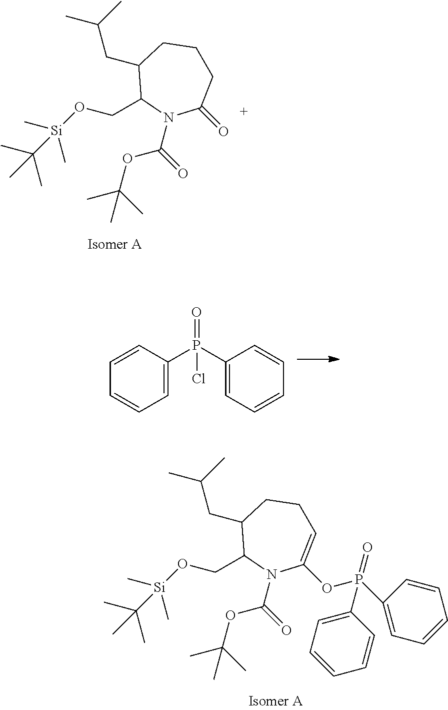 [SMILES: CC(C)CC1CCCC(=O)N(C(=O)OC(C)(C)C)C1CO[Si](C)(C)C(C)(C)C ; O=P(Cl)(c1ccccc1)c1ccccc1 ; CC(C)CC1CCC=C(OP(=O)(c2ccccc2)c2ccccc2)N(C(=O)OC(C)(C)C)C1CO[Si](C)(C)C(C)(C)C]

Into a dried flask was charged with tert-butyl 2-[[tert-butyl(dimethyl)silyl]oxymethyl]-3-isobutyl-7-oxo-azepane-1-carboxylate isomer A (1.323 g, 3.1982 mmol) in anhydrous THF (30 mL). 1.0 M NaHMDS in THE (4 mL of 1 M, 4.0000 mmol) was added to the reaction mixture at −78° C. dropwise. The reaction mixture was stirred at the same temperature for 1 hour. [Chloro(phenyl)phosphoryl]benzene (1.0788 g, 0.87 mL, 4.5589 mmol) was added to the reaction mixture dropwise at −78° C. The reaction mixture was stirred at the same temperature for another 2 hours. The reaction was warmed to rt and quenched with water (30 mL), and extracted with ethyl acetate (3×30 mL). The combined organic layers were washed with brine (30 mL), dried over anhydrous sodium sulfate and concentrated under vacuum. The residue was purified by silica gel chromatography using 0 to 40% ethyl acetate in hexane to furnish tert-butyl 2-[[tert-butyl(dimethyl)silyl]oxymethyl]-7-diphenylphosphoryloxy-3-isobutyl-2,3,4,5-tetrahydroazepine-1-carboxylate isomer A (1.801 g, 87%) as a clear gel. ESI-MS m/z calc. 613.33527, found 614.3 (M+1)⁺; Retention time: 9.41 minutes; LC method S.

### Step 8: Tributyl-(5-isopropoxypyrimidin-2-yl)stannane

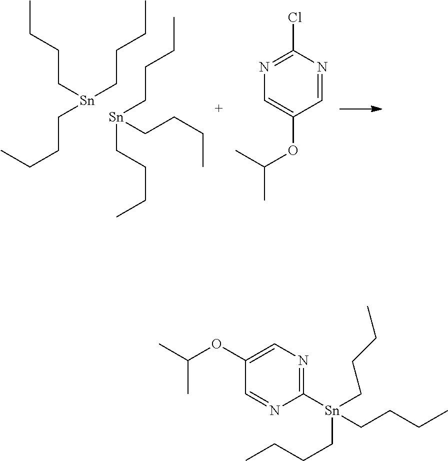

Into a round bottom flask was charged with 2-chloro-5-isopropoxy-pyrimidine (7 g, 40.553 mmol) and Pd(PPh3)₄ (4.7 g, 4.0673 mmol). Degassed xylenes (210 mL) was added to the reaction mixture, followed by tributyl(tributylstannyl)stannane (97.580 g, 85 mL, 168.21 mmol). The reaction was stirred at 135° C. for 16 hours. After cooled to rt, 1 N KF (aq.) (500 mL) was added. The resulting solution was stirred for 1 hour at rt. The resulting solution was filtered through a pad of celite, and the filter cake was washed with ethyl acetate (200 mL). The filtrate's two layers were separated. The organic layer was washed with brine (200 mL), dried over anhydrous sodium sulfate, and concentrated under vacuum. The residue was purified by silica gel chromatography in 10% ethyl acetate in hexane to furnish tributyl-(5-isopropoxypyrimidin-2-yl)stannane (5.12 g, 29%) as a clear liquid, ¹H NMR (500 MHz, DMSO-d6) δ 8.45 (s, 2H), 4.89-4.55 (m, 1H), 1.73-1.36 (m, 6H), 1.32-1.17 (m, 12H), 1.11-0.88 (m, 6H), 0.89-0.57 (m, 9H). ESI-MS m/z calc. 428.18497, found 429.2 (M+1)⁺; Retention time: 4.11 minutes; LC method T.

### Step 9: tert-Butyl 2-(hydroxymethyl)-3-isobutyl-7-(5-isopropoxypyrimidin-2-yl)-2,3,4,5-tetrahydroazepine-1-carboxylate isomer A

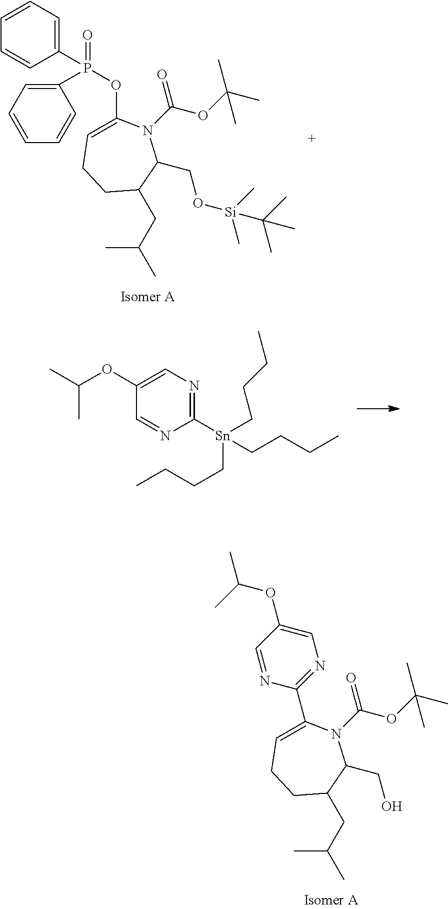

Into a microwavable vial was charged with tert-butyl 2-[[tert-butyl(dimethyl)silyl]oxymethyl]-7-diphenylphosphoryloxy-3-isobutyl-2,3,4,5-tetrahydroazepine-1-carboxylate isomer A (0.88 g, 1.4336 mmol), tributyl-(5-isopropoxypyrimidin-2-yl)stannane (1.1 g, 2.5748 mmol) and anhydrous LiCl (197 mg, 4.6469 mmol) in anhydrous dioxane (15 mL). The reaction mixture was purged with argon for 1 hour. CuI (54 mg, 0.2835 mmol) and Pd(dppf)Cl2 (124 mg, 0.1518 mmol) were added to the reaction mixture. The reaction was purged with argon for another 10 minutes, then the vial was sealed and heated at 125° C. in a microwave reactor for 8 hours. After cooled to rt, the reaction was diluted with 1 N KF (aq.) (15 mL) and stirred for 1 hour. The reaction mixture was filtered through a pad of Celite and washed with ethyl acetate (15 mL). The two layers of the filtrate was separated, and the aqueous layer was extracted with ethyl acetate (3×30 mL). The combined organic layers were washed with brine (15 mL), dried over anhydrous sodium sulfate and concentrated under vacuum. The crude material was dissolved in methanol (30 mL). 1 N HCl (1 mL of 1 M, 1.0000 mmol) was added to the reaction mixture. The reaction was stirred at rt for 1 hour. The reaction mixture was diluted with saturated sodium bicarbonate (50 mL) and ethyl acetate (50 mL). Two layers were separated, and the aqueous layer was extracted with ethyl acetate (2×50 mL). The combined organic layers were washed with brine (50 mL), dried over anhydrous sodium sulfate and concentrated under vacuum. The residue was purified by silica gel chromatography (Gradient: 0 to 15% acetone in hexane) to furnish tert-butyl 2-(hydroxymethyl)-3-isobutyl-7-(5-isopropoxypyrimidin-2-yl)-2,3,4,5-tetrahydroazepine-1-carboxylate isomer A (215 mg, 36%) as a yellow gel. ESI-MS m/z calc. 419.2784, found 420.3 (M+1)⁺; Retention time: 6.74 minutes; LC method S.

### Step 10: [3-Isobutyl-7-(5-isopropoxypyrimidin-2-yl)azepan-2-yl]methanol, isomer A

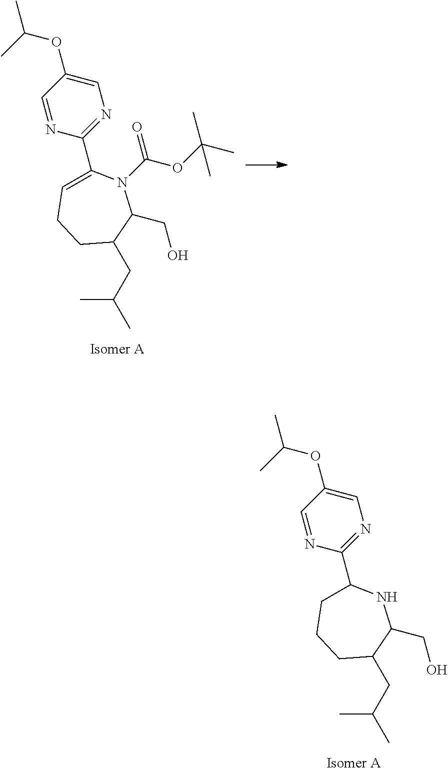 [SMILES: CC(C)CC1CCC=C(c2ncc(OC(C)C)cn2)N(C(=O)OC(C)(C)C)C1CO ; CC(C)CC1CCCC(c2ncc(OC(C)C)cn2)NC1CO]

Into a solution of tert-butyl 2-(hydroxymethyl)-3-isobutyl-7-(5-isopropoxypyrimidin-2-yl)-2,3,4,5-tetrahydroazepine-1-carboxylate isomer A (192 mg, 0.4576 mmol) in DCM (3 mL) was added TFA (2.2200 g, 1.5 mL, 19.470 mmol) dropwise at 0° C. The reaction was stirred at this temperature for 10 minutes and at rt for 1 hour. The solvent was removed in vacuo. The residue was dissolved in anhydrous THE (3 mL). TEA (94.380 mg, 0.13 mL, 0.9327 mmol) and sodium triacetoxyborohydride (398 mg, 1.8779 mmol) were added to the reaction mixture. The reaction was stirred at rt for 1.5 hours. The reaction was quenched with 2 N sodium carbonate (10 mL) and extracted with ethyl acetate (5×10 mL). The combined organic layers were dried over anhydrous sodium sulfate and concentrated under vacuum. The crude material was purified by silica gel chromatography using 0 to 10% methanol in DCM (buffered with 0.3% ammonium hydroxide) to furnish [3-isobutyl-7-(5-isopropoxypyrimidin-2-yl)azepan-2-yl]methanol isomer A (52 mg, 35%) as a yellow gel. ¹H NMR (500 MHz, Chloroform-d) δ 8.31 (s, 2H), 5.79 (s, 1H), 4.72-4.42 (m, 1H), 4.08 (dd, J=10.3, 4.2 Hz, 1H), 3.79 (dd, J=11.1, 4.1 Hz, 1H), 3.48 (t, J=10.4 Hz, 1H), 2.89-2.68 (m, 1H), 2.46-2.23 (m, 1H), 2.02-1.79 (m, 3H), 1.78-1.68 (m, 2H), 1.67-1.48 (m, 3H), 1.42-1.29 (m, 6H), 1.29-1.19 (m, 1H), 1.14-0.99 (m, 1H), 0.89 (d, J=6.5 Hz, 3H), 0.84 (d, J=6.5 Hz, 3H). ESI-MS m/z calc. 321.24164, found 322.3 (M+1)⁺; Retention time: 3.52 minutes; LC method S.

### Step 11: 14-(2,6-Dimethylphenyl)-8-isobutyl-4-(5-isopropoxypyrimidin-2-yl)-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one, isomer A

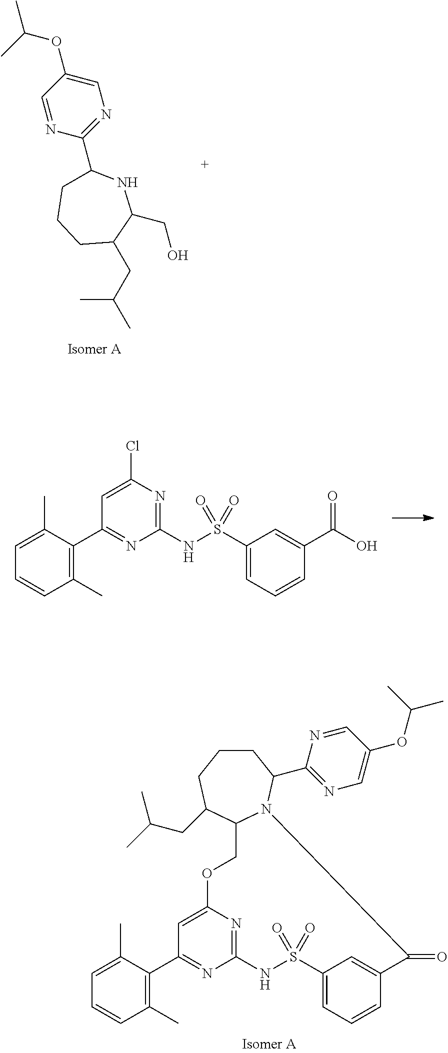 [SMILES: CC(C)CC1CCCC(c2ncc(OC(C)C)cn2)NC1CO ; Cc1cccc(C)c1-c1cc(Cl)nc(NS(=O)(=O)c2cccc(C(=O)O)c2)n1 ; Cc1cccc(C)c1-c1cc2nc(n1)NS(=O)(=O)c1cccc(c1)C(=O)N1C(c3ncc(OC(C)C)cn3)CCCC(CC(C)C)C1CO2]

Into a solution of [3-isobutyl-7-(5-isopropoxypyrimidin-2-yl)azepan-2-yl]methanol isomer A (52 mg, 0.1618 mmol) and 3-[[4-chloro-6-(2,6-dimethylphenyl)pyrimidin-2-yl]sulfamoyl]benzoic acid (80 mg, 0.1825 mmol) in anhydrous THE (1 mL) was added sodium tert-butoxide (80 mg, 0.8324 mmol) at 0° C. The reaction mixture was stirred at rt for 3 hours. The reaction was quenched with 1 N HCl (aq.) (15 mL) at 0° C. and extracted with ethyl acetate (3×15 mL). The combined organic layers were washed with brine (15 mL), dried over anhydrous sodium sulfate and concentrated under vacuum to furnish crude 3-[[4-(2,6-dimethylphenyl)-6-[[3-isobutyl-7-(5-isopropoxypyrimidin-2-yl)azepan-2-yl]methoxy]pyrimidin-2-yl]sulfamoyl]benzoic acid as a yellow gel. ESI-MS m/z calc. 702.32, found 703.3 (M+1)⁺; Retention time: 2.76 minutes. The crude acid was dissolved in anhydrous DMF (10 mL). NMM (92.000 mg, 0.1 mL, 0.9096 mmol) and CDMT (60 mg, 0.3417 mmol) were added to the reaction mixture in this order. The reaction was stirred at rt for 40 hours. The reaction was quenched with 10% citric acid aqueous solution (15 mL) and extracted with ethyl acetate (3×15 mL). The combined organic layers were washed with brine (15 mL), dried over anhydrous sodium sulfate and concentrated under vacuum. The residue was purified by silica gel chromatography using 0 to 50% ethyl acetate in hexane to furnish 14-(2,6-dimethylphenyl)-8-isobutyl-4-(5-isopropoxypyrimidin-2-yl)-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one isomer A (42 mg, 35%) as a white solid. ¹H NMR (500 MHz, Chloroform-d) δ 10.47 (s, 1H), 8.84 (s, 1H), 8.34 (s, 2H), 8.11 (d, J=8.0 Hz, 1H), 7.74 (d, J=7.8 Hz, 1H), 7.57 (t, J=7.7 Hz, 1H), 7.19 (t, J=7.7 Hz, 1H), 7.00 (d, J=7.6 Hz, 2H), 6.14 (s, 1H), 5.84 (dd, J=11.8, 5.2 Hz, 1H), 5.72 (dd, J=13.2, 4.4 Hz, 1H), 4.64 (t, J=11.9 Hz, 1H), 4.60-4.51 (m, 1H), 4.34-4.20 (m, 1H), 2.53 (dt, J=15.3, 5.1 Hz, 1H), 2.02-1.83 (m, 8H), 1.83-1.70 (m, 2H), 1.70-1.61 (m, 1H), 1.35 (d, J=6.0 Hz, 6H), 1.32-1.15 (m, 3H), 0.84-0.74 (m, 1H), 0.74-0.62 (m, 6H). ESI-MS m/z calc. 684.3094, found 685.9 (M+1)⁺; Retention time: 3.25 minutes; LC method W.

### Step 12: 14-(2,6-Dimethylphenyl)-8-isobutyl-4-(5-isopropoxypyrimidin-2-yl)-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one, isomer A, SFC peak 1 (Compound 394), and 14-(2,6-dimethylphenyl)-8-isobutyl-4-(5-isopropoxypyrimidin-2-yl)-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one, isomer A, SFC peak 2 (Compound 395)

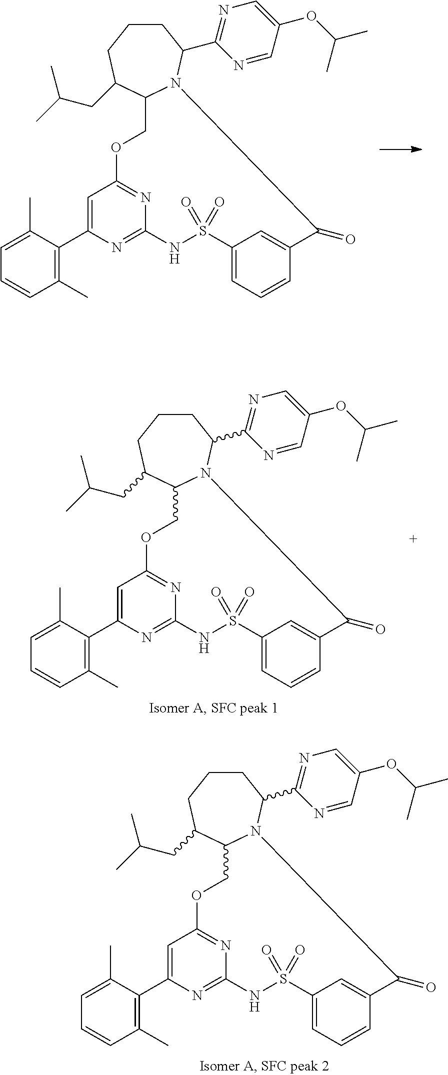 [SMILES: Cc1cccc(C)c1-c1cc2nc(n1)NS(=O)(=O)c1cccc(c1)C(=O)N1C(c3ncc(OC(C)C)cn3)CCCC(CC(C)C)C1CO2 ; Cc1cccc(C)c1-c1cc2nc(n1)NS(=O)(=O)c1cccc(c1)C(=O)N1C(c3ncc(OC(C)C)cn3)CCCC(CC(C)C)C1CO2 ; Cc1cccc(C)c1-c1cc2nc(n1)NS(=O)(=O)c1cccc(c1)C(=O)N1C(c3ncc(OC(C)C)cn3)CCCC(CC(C)C)C1CO2]

14-(2,6-dimethylphenyl)-8-isobutyl-4-(5-isopropoxypyrimidin-2-yl)-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one (42 mg, 0.06133 mmol) (single diastereomer, racemic mixture, from isomer A), was subjected to chiral SFC using a ChiralCel OD column (250×10 mm; Sum) at 50° C. The mobile phase was 22% MeOH (20 mM NH₃), 78% CO₂ at a 20 mL/min flow. Concentration of the sample was ˜21 mg/mL in methanol (no modifier), injection volume 100 μL with an outlet pressure of 214 bar, detection wavelength of 210 nm. For each resulting enantiomer, the solvents were evaporated and the residue was purified by flash chromatography on silica gel (12 g column) using a gradient of methanol (0 to 15% over 15 min) in dichloromethane. The product was triturated in DCM/hexanes and the solvents were evaporated to give as white solids: SFC peak 1, 14-(2,6-dimethylphenyl)-8-isobutyl-4-(5-isopropoxypyrimidin-2-yl)-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one (12 mg, 57%). ¹H NMR (400 MHz, CDCl₃) δ 8.86 (app t, J=1.8 Hz, 1H), 8.34 (s, 2H), 8.15 (d, J=7.9 Hz, 1H), 7.76 (app d, J=7.7 Hz, 1H), 7.63 (t, J=7.8 Hz, 1H), 7.19 (t, J=7.6 Hz, 1H), 7.01 (d, J=7.6 Hz, 2H), 6.17 (s, 1H), 5.84 (dd, J=11.8, 5.2 Hz, 1H), 5.73 (dd, J=13.2, 4.5 Hz, 1H), 4.65 (t, J=11.9 Hz, 1H), 4.57 (hept, J=6.0 Hz, 1H), 4.30 (dt, J=12.1, 4.8 Hz, 1H), 2.59-2.50 (m, 1H), 1.97 (s, 6H), 1.95-1.60 (m, 6H), 1.36 (d, J=6.0 Hz, 6H), 1.34-1.19 (m, 2H), 0.93-0.73 (m, 1H), 0.73-0.66 (m, 6H). ESI-MS m/z calc. 684.3094, found 685.64 (M+1)⁺; Retention time: 2.19 minutes; and SFC peak 2, 14-(2,6-dimethylphenyl)-8-isobutyl-4-(5-isopropoxypyrimidin-2-yl)-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one (11.7 mg, 55%). ¹H NMR (400 MHz, Chloroform-d) δ 8.87 (t, J=1.9 Hz, 1H), 8.34 (s, 2H), 8.13 (d, J=8.0 Hz, 1H), 7.77 (dt, J=7.7, 1.4 Hz, 1H), 7.62 (t, J=7.8 Hz, 1H), 7.19 (t, J=7.6 Hz, 1H), 7.02 (d, J=7.6 Hz, 2H), 6.19 (s, 1H), 5.83 (dd, J=11.7, 5.3 Hz, 1H), 5.73 (dd, J=13.2, 4.5 Hz, 1H), 4.69-4.61 (m, 1H), 4.60-4.53 (m, 1H), 4.35-4.22 (m, 1H), 2.60-2.48 (m, 1H), 2.03-1.84 (m, 9H), 1.78 (t, J=9.2 Hz, 2H), 1.68-1.63 (m, 1H), 1.36 (d, J=6.1, 1.1 Hz, 6H), 1.25-1.17 (m, 2H), 0.82-0.73 (m, 1H), 0.73-0.58 (m, 6H). ESI-MS m/z calc. 684.3094, found 685.81 (M+1)⁺; Retention time: 2.19 minutes; LC method A.

### Example 149: Preparation of Compound 396 and Compound 397

### Step 1: [3-Isobutyl-7-(5-isopropoxypyrimidin-2-yl)-2,3,4,5-tetrahydro-1H-azepin-2-yl]methanol isomer C

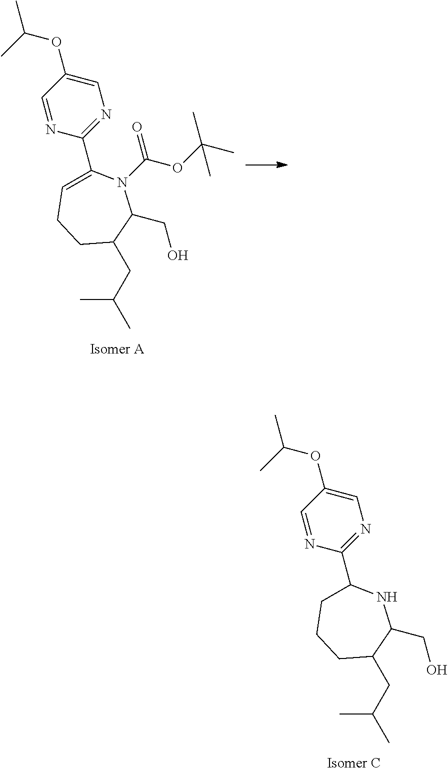 [SMILES: CC(C)CC1CCC=C(c2ncc(OC(C)C)cn2)N(C(=O)OC(C)(C)C)C1CO ; CC(C)CC1CCCC(c2ncc(OC(C)C)cn2)NC1CO]

To a solution of tert-butyl 2-(hydroxymethyl)-3-isobutyl-7-(5-isopropoxypyrimidin-2-yl)-2,3,4,5-tetrahydroazepine-1-carboxylate isomer A (176 mg, 0.4195 mmol) in anhydrous DCM (2.5 mL) was added 2 N HCl in ether (5 mL of 2 M, 10.000 mmol) at rt. The reaction mixture was stirred for 24 hours at rt. LCMS indicated incomplete reaction. Another portion of 2 N HCl in ether (5 mL of 2 M, 10.000 mmol) was added. The reaction was stirred for another 24 hours. The volatiles were moved under vacuum to yield the crude [3-isobutyl-7-(5-isopropoxypyrimidin-2-yl)-2,3,4,5-tetrahydro-1H-azepin-2-yl]methanol (hydrochloride salt) as a yellow solid. ESI-MS m/z calc. 319.226, found 320.1 (M+1)⁺; Retention time: 2.28 minutes. The crude material was dissolved in ethanol (5 mL). 10% Pd/C (100 mg, 10% w/w, 0.0940 mmol) was added to the reaction mixture. The reaction was stirred under hydrogen balloon for 24 hours. The catalyst was removed by filtration through a pad of Celite and washed with ethanol (10 mL). The combined filtrate was concentrated under vacuum. The residue was purified by silica gel chromatography using 0 to 10% methanol in DCM (buffered with 0.3% ammonium hydroxide) to furnish [3-isobutyl-7-(5-isopropoxypyrimidin-2-yl)azepan-2-yl]methanol isomer C (46 mg, 34%) as a yellow gel, which solidified upon standing. ¹H NMR (500 MHz, Chloroform-d) δ 8.31 (s, 2H), 4.73-4.50 (m, 1H), 4.11-3.93 (m, 1H), 3.88-3.49 (m, 3H), 3.38-3.10 (m, 1H), 2.68-2.49 (m, 1H), 2.33-2.08 (m, 1H), 1.90-1.79 (m, 1H), 1.78-1.69 (m, 1H), 1.67-1.59 (m, 2H), 1.56-1.44 (m, 2H), 1.36 (d, J=5.6 Hz, 6H), 1.28-1.25 (m, 1H), 1.03 (m, 1H), 0.97-0.66 (m, 6H). ESI-MS m/z calc. 321.24164, found 322.3 (M+1)⁺; Retention time: 2.21 minutes; LC method T.

### Step 2: 14-(2,6-Dimethylphenyl)-8-isobutyl-4-(5-isopropoxypyrimidin-2-yl)-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one isomer C

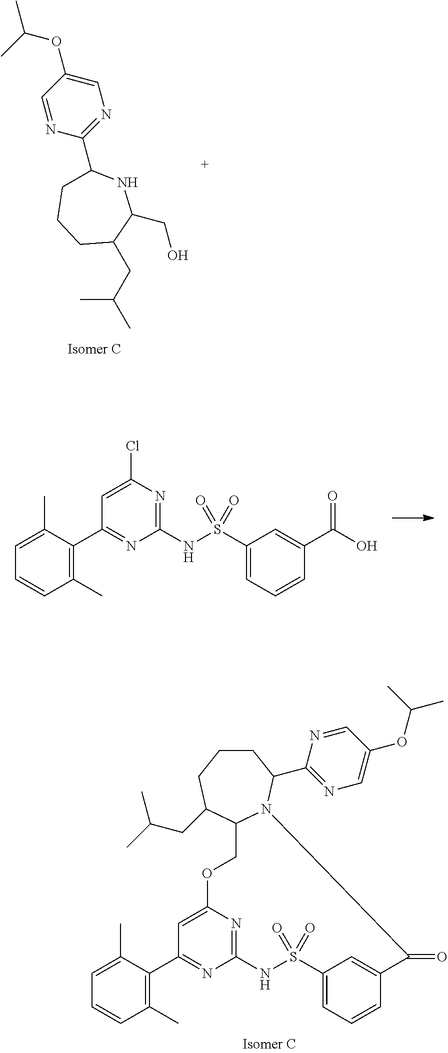 [SMILES: CC(C)CC1CCCC(c2ncc(OC(C)C)cn2)NC1CO ; Cc1cccc(C)c1-c1cc(Cl)nc(NS(=O)(=O)c2cccc(C(=O)O)c2)n1 ; Cc1cccc(C)c1-c1cc2nc(n1)NS(=O)(=O)c1cccc(c1)C(=O)N1C(c3ncc(OC(C)C)cn3)CCCC(CC(C)C)C1CO2]

To a solution of 3-[[4-chloro-6-(2,6-dimethylphenyl)pyrimidin-2-yl]sulfamoyl]benzoic acid (63 mg, 0.1508 mmol) and [3-isobutyl-7-(5-isopropoxypyrimidin-2-yl)azepan-2-yl]methanol isomer C (46 mg, 0.1431 mmol) in anhydrous THF (1 mL) was added sodium tert-butoxide (75 mg, 0.7804 mmol) at 0° C. The reaction mixture was stirred at rt for 3 hours. The reaction was quenched with 1 N HCl (aq.) (15 mL) at 0° C., and then it was extracted with ethyl acetate (3×15 mL). The combined organic layers were washed with brine (15 mL), dried over anhydrous sodium sulfate and concentrated under vacuum to furnish the crude 3-[[4-(2,6-dimethylphenyl)-6-[[3-isobutyl-7-(5-isopropoxypyrimidin-2-yl)azepan-2-yl]methoxy]pyrimidin-2-yl]sulfamoyl]benzoic acid (hydrochloride salt) as an off-white solid. ESI-MS m/z calc. 702.32, found 703.5 (M+1)⁺; Retention time: 2.69 minutes. The crude acid was dissolved in anhydrous DMF (10 mL). NMM (73.600 mg, 0.08 mL, 0.7277 mmol) and CDMT (60 mg, 0.3417 mmol) were added to the reaction mixture. The reaction was stirred at rt for 2 days. The reaction was diluted with 10% citric acid (aq.) (30 mL) and extracted with ethyl acetate (3×30 mL). The combined organic layers were washed with brine (3×30 mL), dried over anhydrous sodium sulfate and concentrated under vacuum. The residue was purified by silica gel chromatography using 25 to 75% ethyl acetate in hexane to furnish 14-(2,6-dimethylphenyl)-8-isobutyl-4-(5-isopropoxypyrimidin-2-yl)-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one (40.9 mg, 41%) (isomer C) as a white solid. ESI-MS m/z calc. 684.3094, found 685.6 (M+1)⁺; Retention time: 3.37 minutes; LC method W.

### Step 3: 14-(2,6-Dimethylphenyl)-8-isobutyl-4-(5-isopropoxypyrimidin-2-yl)-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one, isomer C, SFC peak 1 (Compound 396) and 14-(2,6-dimethylphenyl)-8-isobutyl-4-(5-isopropoxypyrimidin-2-yl)-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one, isomer C, SFC peak 2 (Compound 397)

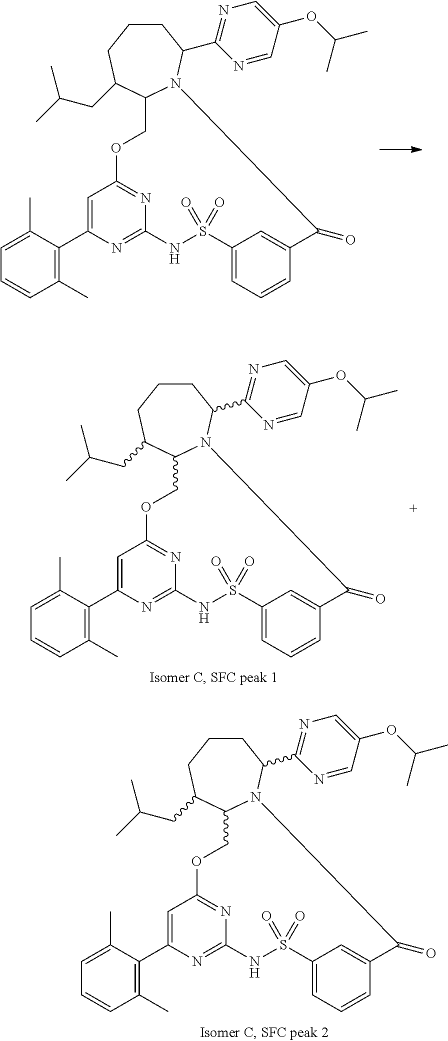 [SMILES: Cc1cccc(C)c1-c1cc2nc(n1)NS(=O)(=O)c1cccc(c1)C(=O)N1C(c3ncc(OC(C)C)cn3)CCCC(CC(C)C)C1CO2 ; Cc1cccc(C)c1-c1cc2nc(n1)NS(=O)(=O)c1cccc(c1)C(=O)N1C(c3ncc(OC(C)C)cn3)CCCC(CC(C)C)C1CO2 ; Cc1cccc(C)c1-c1cc2nc(n1)NS(=O)(=O)c1cccc(c1)C(=O)N1C(c3ncc(OC(C)C)cn3)CCCC(CC(C)C)C1CO2]

14-(2,6-Dimethylphenyl)-8-isobutyl-4-(5-isopropoxypyrimidin-2-yl)-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one (40.8 mg, 0.05958 mmol) (single diastereomer, racemic mixture, from isomer C), was subjected to chiral SFC using a ChiralPak IC column (250×21.2 mm, 5 μM) at 50° C. The mobile phase was 38% MeOH (20 mM NH₃), 62% CO₂ at a 70 mL/min flow in isocratic mode. Concentration of the sample was 12.7 mg/mL in methanol, injection volume 800 μL with an outlet pressure of 202 bar, detection wavelength of 210 nm. For each resulting enantiomer, the solvents were evaporated to give two products as white solids: Isomer C, SFC peak 1, 14-(2,6-dimethylphenyl)-8-isobutyl-4-(5-isopropoxypyrimidin-2-yl)-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one (15.8 mg, 77%), ¹H NMR (400 MHz, Chloroform-d) δ 8.82 (s, 1H), 8.34 (s, 2H), 8.13 (d, J=8.0 Hz, 1H), 7.71 (d, J=7.6 Hz, 1H), 7.53 (broad s, 1H), 7.14 (t, J=7.6 Hz, 1H), 6.96 (d, J=7.6 Hz, 2H), 6.09 (s, 1H), 5.85 (dd, J=11.7, 5.2 Hz, 1H), 5.71 (dd, J=13.1, 4.4 Hz, 1H), 4.73-4.50 (m, 2H), 4.29 (dt, J=12.1, 4.9 Hz, 1H), 2.52 (dt, J=14.3, 4.0 Hz, 1H), 2.02-1.84 (m, 9H), 1.81-1.71 (m, 2H), 1.70-1.59 (m, 1H), 1.36 (d, J=6.0 Hz, 6H), 1.30-1.18 (m, 2H), 0.82-0.75 (m, 1H), 0.74-0.65 (m, 6H). ESI-MS m/z calc. 684.3094, found 685.77 (M+1)⁺; Retention time: 2.17 minutes, and isomer C, SFC peak 2, 14-(2,6-dimethylphenyl)-8-isobutyl-4-(5-isopropoxypyrimidin-2-yl)-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one (15.5 mg, 76%). ¹H NMR (400 MHz, Chloroform-d) δ 8.84 (s, 1H), 8.33 (s, 2H), 8.19 (d, J=7.9 Hz, 1H), 7.74 (d, J=8.0 Hz, 1H), 7.62 (t, J=7.7 Hz, 1H), 7.17 (t, J=7.6 Hz, 1H), 6.98 (d, J=7.7 Hz, 2H), 6.12 (s, 1H), 5.85 (dd, J=11.7, 5.2 Hz, 1H), 5.71 (dd, J=13.2, 4.5 Hz, 1H), 4.64 (t, J=11.9 Hz, 1H), 4.57 (h, J 6.0 Hz, 1H), 4.29 (dt, J=12.1, 4.9 Hz, 1H), 2.53 (dt, J=15.2, 5.2 Hz, 1H), 2.07-1.83 (m, 9H), 1.80-1.71 (m, 2H), 1.67 (dd, J=11.7, 5.6 Hz, 1H), 1.36 (d, J=6.0 Hz, 6H), 1.28 (dt, J=14.5, 5.2 Hz, 2H), 0.85-0.76 (m, 1H), 0.75-0.67 (m, 6H). ESI-MS m/z calc. 684.3094, found 685.83 (M+1)⁺; Retention time: 2.17 minutes; LC method A.

### Example 150: Preparation of Compound 398 and Compound 399

### Step 1: tert-Butyl 2-[[tert-butyl(dimethyl)silyl]oxymethyl]-7-diphenyl phosphoryloxy-3-isobutyl-2,3,4,5-tetrahydroazepine-1-carboxylate isomer B

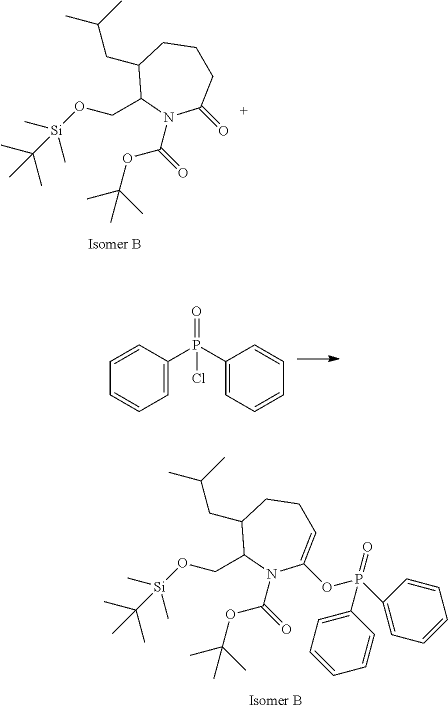 [SMILES: CC(C)CC1CCCC(=O)N(C(=O)OC(C)(C)C)C1CO[Si](C)(C)C(C)(C)C ; O=P(Cl)(c1ccccc1)c1ccccc1 ; CC(C)CC1CCC=C(OP(=O)(c2ccccc2)c2ccccc2)N(C(=O)OC(C)(C)C)C1CO[Si](C)(C)C(C)(C)C]

Into a dried reaction flask was charged with tert-butyl 2-[[tert-butyl(dimethyl)silyl]oxymethyl]-3-isobutyl-7-oxo-azepane-1-carboxylate isomer B (1.261 g, 3.0483 mmol) in anhydrous THF (30 mL). 1.0 M NaHMDS in THE (3.8 mL of 1 M, 3.8000 mmol) was added to the reaction mixture at −78° C. The reaction was stirred at the same temperature for 1 hour. [chloro(phenyl)phosphoryl]benzene (1.0292 g, 0.83 mL, 4.3493 mmol) was added to the reaction mixture dropwise. The reaction was stirred at the same temperature for another 2 hours. The reaction was warmed to rt and quenched with water (30 mL), and extracted with ethyl acetate (3×30 mL). The combined organic layers were washed with brine (30 mL), dried over anhydrous sodium sulfate and concentrated under vacuum. The residue was purified by silica gel chromatography using 0 to 40% ethyl acetate in hexane to furnish tert-butyl 2-[[tert-butyl(dimethyl)silyl]oxymethyl]-7-diphenylphosphoryloxy-3-isobutyl-2,3,4,5-tetrahydroazepine-1-carboxylate isomer B (1.736 g, 93%) as a clear gel. ¹H NMR (500 MHz, Chloroform-d) δ 8.04-7.80 (m, 4H), 7.64-7.33 (m, 6H), 5.38-5.15 (m, 1H), 4.51-4.22 (m, 1H), 3.96-3.79 (m, 1H), 3.76-3.57 (m, 1H), 2.05-1.85 (m, 2H), 1.82-1.64 (m, 2H), 1.56-1.28 (m, 10H), 1.24-1.11 (m, 1H), 1.10-1.00 (m, 1H), 0.91 (m, 6H), 0.89-0.75 (m, 9H), 0.78-0.64 (m, 1H), 0.21-0.00 (m, 6H). ESI-MS m/z calc. 613.33527, found 614.1 (M+1)⁺; Retention time: 9.45 minutes; LC method S.

### Step 2: tert-Butyl 2-(hydroxymethyl)-3-isobutyl-7-(5-isopropoxypyrimidin-2-yl)-2,3,4,5-tetrahydroazepine-1-carboxylate isomer B

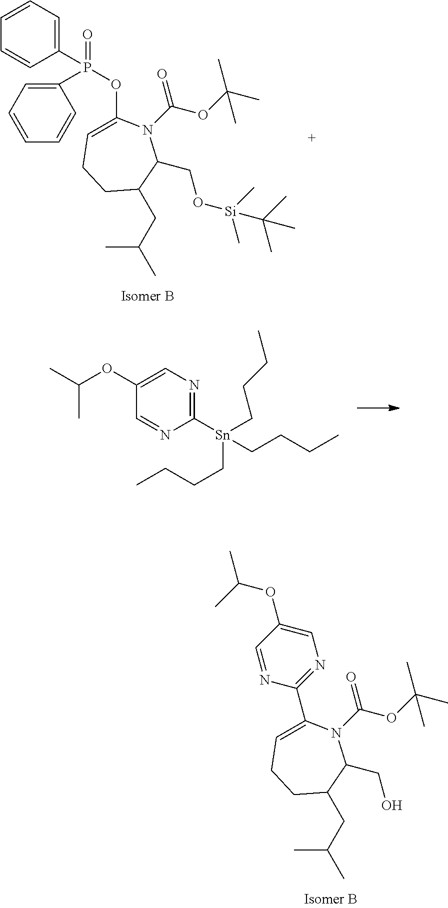

Into a microwavable vial was charged with tert-butyl 2-[[tert-butyl(dimethyl)silyl]oxymethyl]-7-diphenylphosphoryloxy-3-isobutyl-2,3,4,5-tetrahydroazepine-1-carboxylate (868 mg, 1.4140 mmol), tributyl-(5-isopropoxy pyrimidin-2-yl)stannane (1.195 g, 2.7972 mmol) and anhydrous LiCl (164 mg, 3.8685 mmol) in anhydrous dioxane (15 mL). The reaction mixture was purged with nitrogen for 1 hour. CuI (54 mg, 0.2835 mmol) and Pd(dppf)Cl₂ (119 mg, 0.1457 mmol) were added to the reaction mixture. The reaction was purged with argon for another 10 minutes, then the vial was sealed and heated at 125° C. in a microwave reactor for 8 hours. After cooled to rt, the reaction was diluted with 1 N KF (aq.) (15 mL) and stirred for 1 hour. The reaction mixture was filtered through a pad of Celite and washed with ethyl acetate (15 mL). The two layers of the filtrate were separated, and the aqueous layer was extracted with ethyl acetate (3×30 mL). The combined organic layers were washed with brine (15 mL), dried over anhydrous sodium sulfate and concentrated under vacuum. The crude material was dissolved in methanol (15 mL). 1 N HCl (1 mL of 1 M, 1.0000 mmol) was added to the reaction mixture. The reaction was stirred at rt for 1 hour. The reaction mixture was diluted with saturated sodium bicarbonate (50 mL) and ethyl acetate (50 mL). Two layers were separated and the aqueous layer was extracted with ethyl acetate (2×50 mL). The combined organic layers were washed with brine (50 mL), dried over anhydrous sodium sulfate and concentrated under vacuum. The residue was purified by silica gel chromatography (Gradient: 0 to 20% acetone in hexane) to furnish tert-butyl 2-(hydroxymethyl)-3-isobutyl-7-(5-isopropoxypyrimidin-2-yl)-2,3,4,5-tetrahydroazepine-1-carboxylate isomer B (202 mg, 27%) as a yellow gel. ESI-MS m/z calc. 419.2784, found 420.3 (M+1)⁺; Retention time: 3.81 minutes

### Step 3: [3-Isobutyl-7-(5-isopropoxypyrimidin-2-yl)azepan-2-yl]methanol isomer B

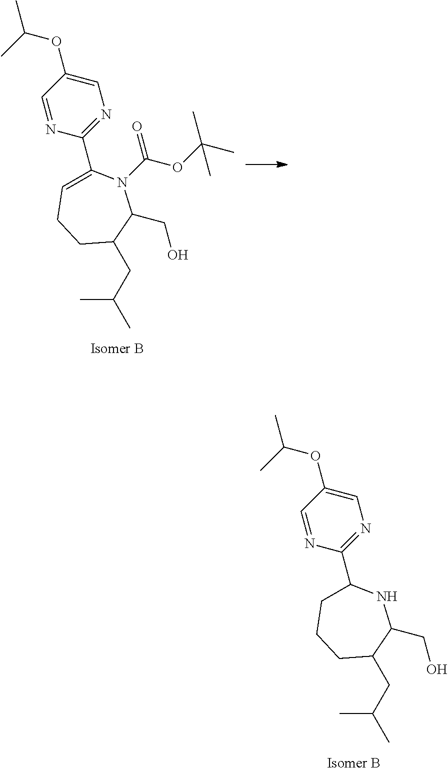 [SMILES: CC(C)CC1CCC=C(c2ncc(OC(C)C)cn2)N(C(=O)OC(C)(C)C)C1CO ; CC(C)CC1CCCC(c2ncc(OC(C)C)cn2)NC1CO]

Into a solution of tert-butyl 2-(hydroxymethyl)-3-isobutyl-7-(5-isopropoxypyrimidin-2-yl)-2,3,4,5-tetrahydroazepine-1-carboxylate isomer B (202 mg, 0.3852 mmol) in anhydrous DCM (3 mL) was added TFA (2.2200 g, 1.5 mL, 19.470 mmol) dropwise at 0° C. The reaction was stirred at rt for 1 hour. The solvent was removed in vacuo. To the residue was added 2 N HCl in ether (1.5 mL of 2 M, 3.0000 mmol). The volatiles were removed under vacuum overnight to furnish crude [3-isobutyl-7-(5-isopropoxypyrimidin-2-yl)-2,3,4,5-tetrahydro-1H-azepin-2-yl]methanol (hydrochloride salt) as a yellow oil. The crude material was dissolved in anhydrous THE (3 mL). TEA (79.860 mg, 0.11 mL, 0.7892 mmol) and sodium triacetoxyborohydride (408 mg, 1.9251 mmol) were added to the reaction mixture. The reaction was stirred at rt for 1.5 hours. The reaction was quenched with 2 N sodium carbonate (10 mL) and extracted with ethyl acetate (5×10 mL). The combined organic layers were dried over anhydrous sodium sulfate and concentrated under vacuum. The crude material was purified by silica gel chromatography using 0 to 10% methanol in DCM (buffered with 0.3% ammonium hydroxide) to furnish [3-isobutyl-7-(5-isopropoxypyrimidin-2-yl)azepan-2-yl]methanol isomer B (24 mg, 19%) as a yellow gel. ¹H NMR (500 MHz, Chloroform-d) δ 8.32 (s, 2H), 4.67-4.50 (m, 1H), 4.31-4.16 (m, 1H), 3.57-3.46 (m, 2H), 3.45 (s, 2H), 3.19-3.03 (m, 1H), 2.26-2.12 (m, 1H), 1.99-1.89 (m, 1H), 1.89-1.81 (m, 1H), 1.68-1.54 (m, 4H), 1.54-1.44 (m, 1H), 1.43-1.29 (m, 6H), 1.28-1.17 (m, 1H), 1.05 (ddd, J=13.2, 9.6, 3.5 Hz, 1H), 0.93 (dd, J=9.3, 6.6 Hz, 3H), 0.84 (dd, J=9.2, 6.5 Hz, 3H). ESI-MS m/z calc. 321.24164, found 322.4 (M+1)⁺; Retention time: 2.25 minutes; LC method T.

### Step 4: 14-(2,6-Dimethylphenyl)-8-isobutyl-4-(5-isopropoxypyrimidin-2-yl)-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one, isomer B

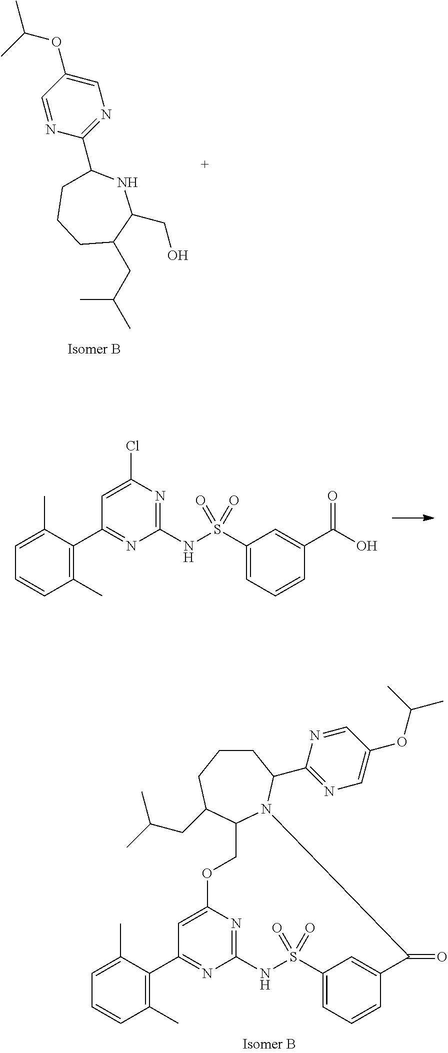 [SMILES: CC(C)CC1CCCC(c2ncc(OC(C)C)cn2)NC1CO ; Cc1cccc(C)c1-c1cc(Cl)nc(NS(=O)(=O)c2cccc(C(=O)O)c2)n1 ; Cc1cccc(C)c1-c1cc2nc(n1)NS(=O)(=O)c1cccc(c1)C(=O)N1C(c3ncc(OC(C)C)cn3)CCCC(CC(C)C)C1CO2]

Into a solution of 3-[[4-chloro-6-(2,6-dimethylphenyl)pyrimidin-2-yl]sulfamoyl]benzoic acid (65 mg, 0.1482 mmol) and [3-isobutyl-7-(5-isopropoxy pyrimidin-2-yl)azepan-2-yl]methanol isomer B (44 mg, 0.1369 mmol) in anhydrous THF (1 mL) was added sodium tert-butoxide (68 mg, 0.7076 mmol) at 0° C. The reaction was stirred at rt for 1.5 hours. The reaction was quenched with 1 N HCl (aq.) (15 mL) at 0° C. and extracted with ethyl acetate (3×15 mL). The combined organic layers were washed with brine (15 mL), dried over anhydrous sodium sulfate and concentrated under vacuum to furnish the crude 3-[[4-(2,6-dimethylphenyl)-6-[[3-isobutyl-7-(5-isopropoxypyrimidin-2-yl)azepan-2-yl]methoxy]pyrimidin-2-yl]sulfamoyl]benzoic acid (hydrochloride salt) as a yellow gel. ESI-MS m/z calc. 702.32, found 703.5 (M+1)⁺; Retention time: 2.76 minutes. The crude material was dissolved in anhydrous DMF (10 mL). NMM (69.000 mg, 0.075 mL, 0.6822 mmol) was added to the reaction mixture, followed by CDMT (48 mg, 0.2734 mmol). The reaction was stirred at rt for 2 days. The reaction was diluted with 10% citric acid (20 mL) and ethyl acetate (20 mL). Two layers were separated, and the aqueous layer was extracted with (2×30 mL). The combined organic layers were washed with brine (3×20 mL), dried over anhydrous sodium sulfate, and concentrated under vacuum. The residue was purified by silica gel chromatography using 0 to 80% ethyl acetate in hexane. The fractions containing the desired product were combined and concentrated under vacuum. The residue was further purified by reverse phase HPLC using 0 to 100% acetonitrile in water (buffered with 0.1% TFA) to furnish 14-(2,6-dimethylphenyl)-8-isobutyl-4-(5-isopropoxypyrimidin-2-yl)-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one (11.6 mg, 12%) (isomer B) as a white powder. ¹H NMR (500 MHz, DMSO-d₆) δ 8.57 (s, 1H), 8.44 (s, 2H), 7.87 (s, 1H), 7.61 (s, 2H), 7.25 (t, J=7.4, 7.4 Hz, 1H), 7.12 (d, J=7.9 Hz, 2H), 6.33 (s, 1H), 5.64 (dd, J=12.0, 4.8 Hz, 1H), 5.46 (dd, J=13.0, 4.6 Hz, 1H), 4.86-4.72 (m, 2H), 4.31 (dd, J=12.1, 5.1 Hz, 1H), 2.35-2.28 (m, 1H), 2.13-1.96 (m, 8H), 1.71-1.52 (m, 4H), 1.28 (dd, J=6.0, 1.6 Hz, 6H), 0.94 (dt, J=13.4, 5.7, 5.7 Hz, 1H), 0.79-0.64 (m, 2H), 0.52 (d, J=6.3 Hz, 3H), 0.42 (d, J=6.3 Hz, 3H). ESI-MS m/z calc. 684.3094, found 685.6 (M+1)⁺; Retention time: 3.31 minutes; LC method W.

### Step 5: 14-(2,6-Dimethylphenyl)-8-isobutyl-4-(5-isopropoxypyrimidin-2-yl)-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one, isomer B, SFC peak 1 (Compound 398), and 14-(2,6-dimethylphenyl)-8-isobutyl-4-(5-isopropoxypyrimidin-2-yl)-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one, isomer B, SFC peak 2 (Compound 399)

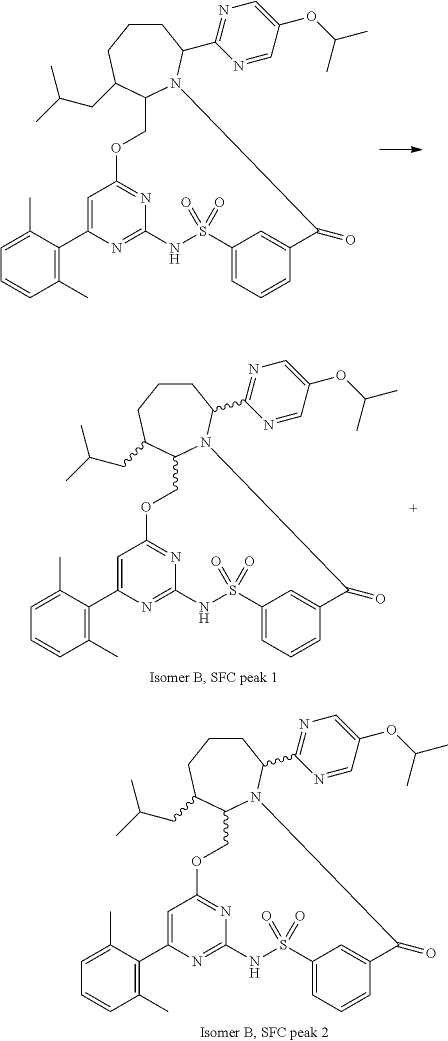 [SMILES: Cc1cccc(C)c1-c1cc2nc(n1)NS(=O)(=O)c1cccc(c1)C(=O)N1C(c3ncc(OC(C)C)cn3)CCCC(CC(C)C)C1CO2 ; Cc1cccc(C)c1-c1cc2nc(n1)NS(=O)(=O)c1cccc(c1)C(=O)N1C(c3ncc(OC(C)C)cn3)CCCC(CC(C)C)C1CO2 ; Cc1cccc(C)c1-c1cc2nc(n1)NS(=O)(=O)c1cccc(c1)C(=O)N1C(c3ncc(OC(C)C)cn3)CCCC(CC(C)C)C1CO2]

14-(2,6-Dimethylphenyl)-8-isobutyl-4-(5-isopropoxypyrimidin-2-yl)-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one (10.4 mg, 0.01519 mmol) (single diastereomer, racemic mixture, from isomer B), was subjected to chiral SFC using a ChiralCel OD column (250×10 mm; 5 m) at 50° C. The mobile phase was 22% MeOH (20 mM NH₃) at a 20 mL/min flow. Concentration of the sample was 14.9 mg/mL in methanol, injection volume 100 μL with a pressure of 181 bar, detection wavelength of 210 nm. For each resulting enantiomer, the solvents were evaporated to give as a colorless glass: isomer B, SFC peak 1, 14-(2,6-dimethylphenyl)-8-isobutyl-4-(5-isopropoxypyrimidin-2-yl)-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one (3.2 mg, 62%). ESI-MS m/z calc. 684.3094, found 685.88 (M+1)⁺; Retention time: 2.15 minutes, and isomer B, SFC peak 2, 14-(2,6-dimethylphenyl)-8-isobutyl-4-(5-isopropoxypyrimidin-2-yl)-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one (4 mg, 77%). ESI-MS m/z calc. 684.3094, found 685.99 (M+1)⁺; Retention time: 2.15 minutes; LC method A.

### Example 151: Preparation of Compound 400 and Compound 401

### Step 1: 6-Bromo-N-cyclobutyl-N-methyl-pyrazin-2-amine

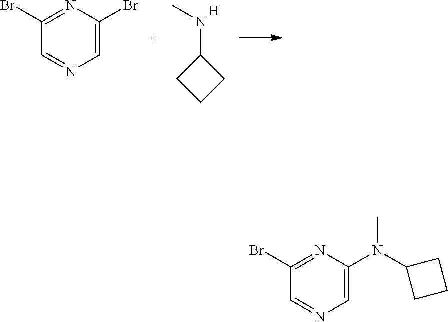

To a solution of 2,6-dibromopyrazine (136 mg, 0.5717 mmol) in anhydrous DMSO (1 mL) was added N-methylcyclobutanamine (hydrochloride salt) (91 mg, 0.7483 mmol) and DIPEA (185.50 mg, 0.25 mL, 1.4353 mmol). The reaction was stirred at rt for 16 hours. The reaction was diluted with ethyl acetate (30 mL) and washed with water (2×10 mL) and brine (10 mL). The organic layer was dried over anhydrous sodium sulfate and concentrated under reduced pressure. The residue was purified by silica gel chromatography using o to 20% ethyl acetate in hexane to furnish 6-bromo-N-cyclobutyl-N-methyl-pyrazin-2-amine (75 mg, 54%) as a clear liquid. ¹H NMR (500 MHz, Chloroform-d) δ 7.98-7.64 (m, 2H), 4.76-4.45 (m, 1H), 3.02 (s, 3H), 2.33-2.22 (m, 2H), 2.22-2.09 (m, 2H), 1.79-1.64 (m, 2H). ESI-MS m/z calc. 241.02145, found 242.2 (M+1)⁺; Retention time: 3.26 minutes; LC method T.

### Step 2: N-Cyclobutyl-N-methyl-6-tributylstannyl-pyrazin-2-amine

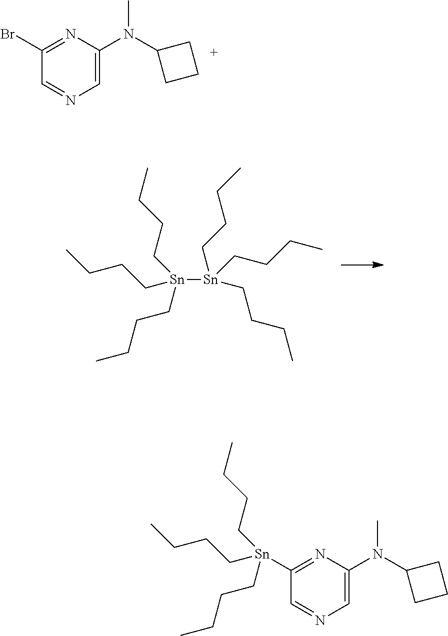 [SMILES: CN(c1cncc(Br)n1)C1CCC1 ; CCC[CH2][Sn]([CH2]CCC)([CH2]CCC)[Sn]([CH2]CCC)([CH2]CCC)[CH2]CCC ; CCC[CH2][Sn]([CH2]CCC)([CH2]CCC)[c]1cncc(N(C)C2CCC2)n1]

Into a sealed tube was charged with 6-bromo-N-cyclobutyl-N-methyl-pyrazin-2-amine (2.12 g, 8.7561 mmol), tributyl(tributylstannyl)stannane (25.256 g, 22 mL, 43.537 mmol), anhydrous LiCl (1.26 g, 29.721 mmol) and Pd(PPh₃)₄ (206 mg, 0.1783 mmol). Degassed dioxane (20 mL) was added to the reaction mixture. The tube was sealed and heated at 120° C. for 1 hour. After cooled to rt, 1 N KF (aq.) (25 mL of 1 M, 25.000 mmol) was added to the reaction mixture. The reaction was stirred at rt for 1 hour. The precipitate was filtrated off through a pad of Celite and washed with ethyl acetate (50 mL). The combined filtrate was washed with brine (2×20 mL), dried over anhydrous sodium sulfate and concentrated under vacuum. The residue was purified by silica gel chromatography (0 to 15% [10% 7N ammonia in methanol/Ethyl acetate] in hexane) to furnish N-cyclobutyl-N-methyl-6-tributylstannyl-pyrazin-2-amine (2.692 g, 68%) as a yellow liquid. ESI-MS m/z calc. 453.21658, found 454.2 (M+1)⁺; Retention time: 4.15 minutes; LC method T.

### Step 3: tert-Butyl 2-[[tert-butyl(dimethyl)silyl]oxymethyl]-7-[6-[cyclobutyl(methyl)amino]pyrazin-2-yl]-3-isobutyl-2,3,4,5-tetrahydroazepine-1-carboxylate isomer A/isomer C mixture

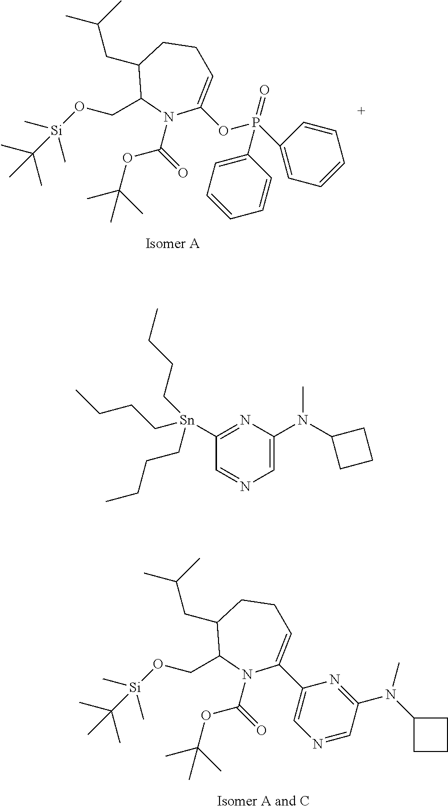 [SMILES: CC(C)CC1CCC=C(OP(=O)(c2ccccc2)c2ccccc2)N(C(=O)OC(C)(C)C)C1CO[Si](C)(C)C(C)(C)C ; CCC[CH2][Sn]([CH2]CCC)([CH2]CCC)[c]1cncc(N(C)C2CCC2)n1 ; CC(C)CC1CCC=C(c2cncc(N(C)C3CCC3)n2)N(C(=O)OC(C)(C)C)C1CO[Si](C)(C)C(C)(C)C]

Into a 20 mL microwavable vial was charged with tert-butyl 2-[[tert-butyl(dimethyl)silyl]oxymethyl]-7-diphenylphosphoryloxy-3-isobutyl-2,3,4,5-tetrahydroazepine-1-carboxylate isomer A (0.941 g, 1.4563 mmol), N-cyclobutyl-N-methyl-6-tributylstannyl-pyrazin-2-amine (987 mg, 2.1824 mmol), CuI (107 mg, 0.5618 mmol), LiCl (219 mg, 5.1658 mmol), Pd(dppf)Cl2 (130 mg, 0.1592 mmol) and degassed dioxane (15 mL). The vial was sealed and irradiated in a microwave reactor at 150° C. for 4 hours. LCMS indicated the starting material left. Another portion of N-cyclobutyl-N-methyl-6-tributylstannyl-pyrazin-2-amine (330 mg, 0.7297 mmol) was added. The reaction was purged with argon for 30 minutes. CuI (58 mg, 0.3045 mmol) and Pd(dppf)Cl2 (126 mg, 0.1543 mmol) were added. The reaction was heated in a microwave at 150° C. for another 3 hours. The reaction was cooled to rt and diluted with 1 N KF (aq.) (15 mL of 1 M, 15.000 mmol) and ethyl acetate (15 mL). The reaction mixture was stirred at rt for 1 hour. The precipitate was removed by filtration through a pad of Celite and washed with ethyl acetate (50 mL). The combined filtrate was washed with brine (2×15 mL), dried over anhydrous sodium sulfate and concentrated under vacuum. The residue was purified by silica gel chromatography using 0 to 30% ethyl acetate in hexane to furnish tert-butyl 2-[[tert-butyl(dimethyl)silyl]oxymethyl]-7-[6-[cyclobutyl(methyl) amino]pyrazin-2-yl]-3-isobutyl-2,3,4,5-tetrahydroazepine-1-carboxylate (mixture of isomer A and C) (89 mg, 10%) as a yellow liquid. ¹H NMR (500 MHz, Chloroform-d) δ 8.08-7.90 (m, 1H), 7.87-7.69 (m, 1H), 6.66-6.22 (m, 1H), 4.73-4.57 (m, 1H), 4.56-4.18 (m, 1H), 3.90-3.70 (m, 1H), 3.70-3.48 (m, 1H), 3.11-2.87 (m, 3H), 2.63-2.35 (m, 1H), 2.29-2.20 (m, 2H), 2.20-2.11 (m, 2H), 1.98-1.87 (m, 1H), 1.76-1.69 (m, 2H), 1.66-1.61 (m, 1H), 1.60-1.55 (m, 1H), 1.42 (s, 2H), 1.42-1.32 (m, 2H), 1.31-1.24 (m, 2H), 1.12 (s, 7H), 0.97-0.73 (m, 15H), 0.13-−0.08 (m, 6H). ESI-MS m/z calc. 558.39655, found 559.4 (M+1)⁺; Retention time: 4.84 minutes; LC method T.

### Step 4: [7-[6-[Cyclobutyl(methyl)amino]pyrazin-2-yl]-3-isobutyl-azepan-2-yl]methanol isomer A/isomer C mixture

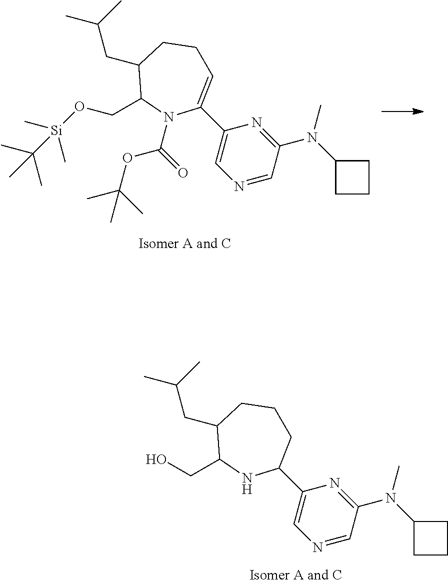 [SMILES: CC(C)CC1CCC=C(c2cncc(N(C)C3CCC3)n2)N(C(=O)OC(C)(C)C)C1CO[Si](C)(C)C(C)(C)C ; CC(C)CC1CCCC(c2cncc(N(C)C3CCC3)n2)NC1CO]

Into a reaction flask was charged with tert-butyl 2-[[tert-butyl(dimethyl)silyl]oxymethyl]-7-[6-[cyclobutyl(methyl)amino]pyrazin-2-yl]-3-isobutyl-2,3,4,5-tetrahydroazepine-1-carboxylate (isomer A and C mixture) (89 mg, 0.1513 mmol) and 4 N HCl in dioxane (3 mL of 4 M, 12.000 mmol). The reaction was stirred at rt overnight. The volatiles were removed under vacuum to furnish [7-[6-[cyclobutyl(methyl)amino]pyrazin-2-yl]-3-isobutyl-2,3,4,5-tetrahydro-1H-azepin-2-yl]methanol as a dark brown solid. ESI-MS m/z calc. 344.2576, found 345.2 (M+1)⁺; Retention time: 2.32 minutes The crude material was dissolved in anhydrous THE (3 mL). triethylamine (30.492 mg, 0.042 mL, 0.3013 mmol) and sodium triacetoxyborohydride (140 mg, 0.6606 mmol) were added to the reaction mixture. The reaction was stirred at rt for 2 hours. The reaction was quenched with 2 N sodium carbonate aqueous solution (10 mL), and extracted with ethyl acetate (5×10 mL). The combined organic layers were washed with brine (10 mL), dried over anhydrous sodium sulfate and concentrated under vacuum. The residue was purified by silica gel chromatography using 0 to 10% methanol in DCM (buffered with 0.3% ammonium hydroxide) to furnish [7-[6-[cyclobutyl(methyl) amino]pyrazin-2-yl]-3-isobutyl-azepan-2-yl]methanol (isomer A and C mixture) (40 mg, 76%) as a brown gel. ¹H NMR (500 MHz, Chloroform-d) δ 7.88-7.81 (m, 1H), 7.80-7.72 (m, 1H), 4.69-4.51 (m, 1H), 3.84-3.65 (m, 2H), 3.41-3.14 (m, 1H), 3.08-2.96 (m, 3H), 2.81-2.51 (m, 1H), 2.29-2.21 (m, 2H), 2.21-2.08 (m, 3H), 1.86-1.68 (m, 4H), 1.66-1.44 (m, 3H), 1.39-0.98 (m, 4H), 0.95-0.70 (m, 6H). ESI-MS m/z calc. 346.27325, found 347.3 (M+1)⁺; Retention time: 2.26 minutes; LC method T.

### Step 5: 4-[6-[Cyclobutyl(methyl)amino]pyrazin-2-yl]-14-(2,6-dimethylphenyl)-8-isobutyl-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one isomer A/isomer C mixture

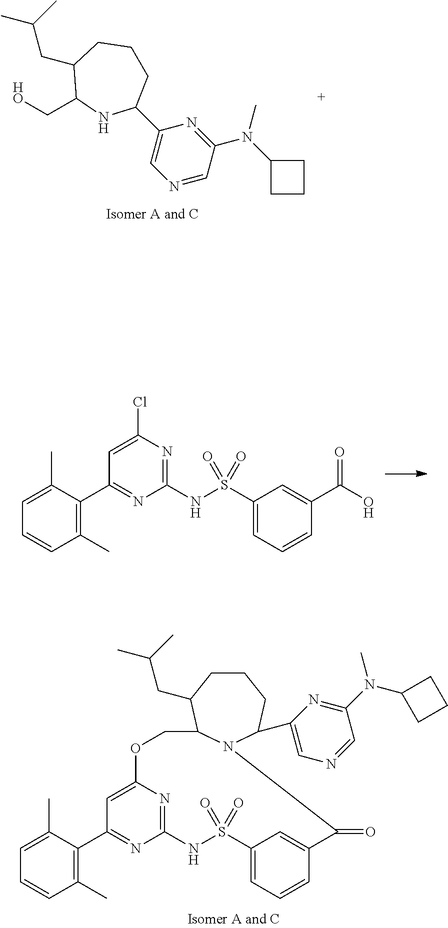 [SMILES: CC(C)CC1CCCC(c2cncc(N(C)C3CCC3)n2)NC1CO ; Cc1cccc(C)c1-c1cc(Cl)nc(NS(=O)(=O)c2cccc(C(=O)O)c2)n1 ; Cc1cccc(C)c1-c1cc2nc(n1)NS(=O)(=O)c1cccc(c1)C(=O)N1C(c3cncc(N(C)C4CCC4)n3)CCCC(CC(C)C)C1CO2]

Into a reaction vial was charged with [7-[6-[cyclobutyl(methyl)amino]pyrazin-2-yl]-3-isobutyl-azepan-2-yl]methanol isomer A (40 mg, 0.1154 mmol) and 3-[[4-chloro-6-(2,6-dimethylphenyl)pyrimidin-2-yl]sulfamoyl]benzoic acid (isomer A and C mixture) (56 mg, 0.1340 mmol) in anhydrous THF (0.5 mL). Sodium tert-butoxide (78 mg, 0.8116 mmol) was added to the reaction mixture at rt. The reaction was stirred at rt for 1 hour. The reaction was diluted with ethyl acetate (15 mL) and 1 N HCl (aq.) (15 mL). Two layers were separated, and the aqueous layer was extracted with ethyl acetate (5×15 mL). The combine organic layers were washed with brine (15 mL), dried over anhydrous sodium sulfate and concentrated under vacuum to furnish the crude 3-[[4-[[7-[6-[cyclobutyl(methyl)amino]pyrazin-2-yl]-3-isobutyl-azepan-2-yl]methoxy]-6-(2,6-dimethylphenyl)pyrimidin-2-yl]sulfamoyl]benzoic acid (hydrochloride salt) as a yellow solid. ESI-MS m/z calc. 727.3516, found 728.5 (M+1)⁺; Retention time: 2.66 minutes. The crude material was dissolved in anhydrous DMF (7.5 mL). CDMT (45 mg, 0.2563 mmol) and NMM (57.960 mg, 0.063 mL, 0.5730 mmol) was added to the reaction mixture. The reaction was stirred at rt for 2 hours. Another portion of CDMT (33 mg, 0.1880 mmol) and NMM (34.960 mg, 0.038 mL, 0.3456 mmol) was added to the reaction mixture. The reaction was stirred at rt for 2 days, then it was quenched with 10% citric acid (20 mL). The reaction was extracted with ethyl acetate (3×20 mL). The combined organic layers were washed with brine (3×20 mL), dried over anhydrous sodium sulfate and concentrated under vacuum. The crude material was combined with the crude material from another experiment run on 101 mg of starting reactant for further separation of isomer A and C. 4-[6-[cyclobutyl(methyl)amino]pyrazin-2-yl]-14-(2,6-dimethylphenyl)-8-isobutyl-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one (isomer A and C) ESI-MS m/z calc. 709.341, found 710.6 (M+1)⁺; Retention time: 5.99 minutes and 5.73 minutes; LC method T.

### Step 6: 4-[6-[Cyclobutyl(methyl)amino]pyrazin-2-yl]-14-(2,6-dimethylphenyl)-8-isobutyl-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one, isomer A and 4-[6-[cyclobutyl(methyl)amino]pyrazin-2-yl]-14-(2,6-dimethylphenyl)-8-isobutyl-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one (1:1 mixture of isomer A and C)

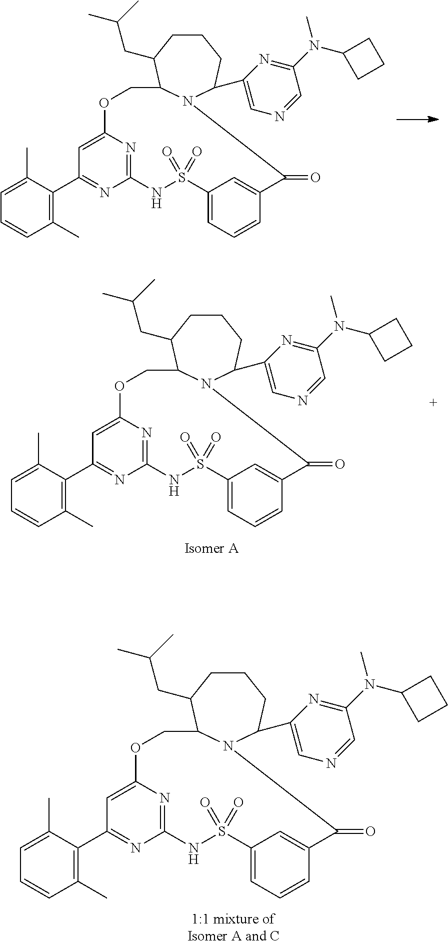 [SMILES: Cc1cccc(C)c1-c1cc2nc(n1)NS(=O)(=O)c1cccc(c1)C(=O)N1C(c3cncc(N(C)C4CCC4)n3)CCCC(CC(C)C)C1CO2 ; Cc1cccc(C)c1-c1cc2nc(n1)NS(=O)(=O)c1cccc(c1)C(=O)N1C(c3cncc(N(C)C4CCC4)n3)CCCC(CC(C)C)C1CO2 ; Cc1cccc(C)c1-c1cc2nc(n1)NS(=O)(=O)c1cccc(c1)C(=O)N1C(c3cncc(N(C)C4CCC4)n3)CCCC(CC(C)C)C1CO2]

Two crude materials 4-[6-[cyclobutyl(methyl)amino]pyrazin-2-yl]-14-(2,6-dimethylphenyl)-8-isobutyl-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one (prepared using 40 mg of starting reactant) and 4-[6-[cyclobutyl(methyl)amino]pyrazin-2-yl]-14-(2,6-dimethylphenyl)-8-isobutyl-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one (prepared using 101 mg of starting reactant) were combined and purified by silica gel chromatography using 0 to 80% ethyl acetate in hexane to furnish 4-[6-[cyclobutyl(methyl)amino]pyrazin-2-yl]-14-(2,6-dimethylphenyl)-8-isobutyl-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one (28 mg) as a yellow solid (pure isomer A) and 4-[6-[cyclobutyl(methyl)amino]pyrazin-2-yl]-14-(2,6-dimethylphenyl)-8-isobutyl-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one (58 mg) as an orange gel (1:1 mixture of isomer A and C). Isomer A: ESI-MS m/z calc. 709.341, found 710.8 (M+1)⁺; Retention time: 2.91 minutes. Isomer C ESI-MS m/z calc. 709.341, found 710.5 (M+1)⁺; Retention time: 2.8 minutes, 2.91 minutes; LC method W.

### Step 7: 4-[6-[Cyclobutyl(methyl)amino]pyrazin-2-yl]-14-(2,6-dimethylphenyl)-8-isobutyl-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one, isomer A, SFC peak 1 (Compound 400), and 4-[6-[cyclobutyl(methyl)amino]pyrazin-2-yl]-14-(2,6-dimethylphenyl)-8-isobutyl-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one, isomer A, SFC peak 2 (Compound 401)

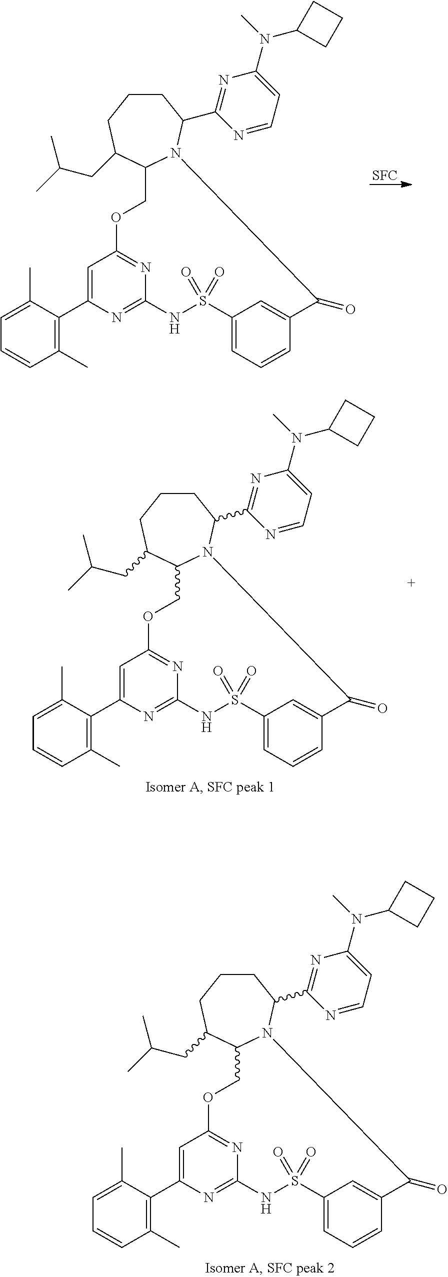 [SMILES: Cc1cccc(C)c1-c1cc2nc(n1)NS(=O)(=O)c1cccc(c1)C(=O)N1C(c3nccc(N(C)C4CCC4)n3)CCCC(CC(C)C)C1CO2 ; "SFC" ; Cc1cccc(C)c1-c1cc2nc(n1)NS(=O)(=O)c1cccc(c1)C(=O)N1C(c3nccc(N(C)C4CCC4)n3)CCCC(CC(C)C)C1CO2 ; Cc1cccc(C)c1-c1cc2nc(n1)NS(=O)(=O)c1cccc(c1)C(=O)N1C(c3nccc(N(C)C4CCC4)n3)CCCC(CC(C)C)C1CO2]

4-[6-[Cyclobutyl(methyl)amino]pyrazin-2-yl]-14-(2,6-dimethylphenyl)-8-isobutyl-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one (27 mg, 0.03803 mmol) (single diastereomer A, racemic mixture), was subjected to chiral SFC using a ChiralPak AS column (250×21.2 mm; Sum) at 50° C. The mobile phase was 44% MeOH (20 mM NH₃) at a 70 mL/min flow. Concentration of the sample was ˜18 mg/mL in methanol (no modifier), injection volume 500 L with an outlet pressure of 181 bar, detection wavelength of 210 nm. For each resulting enantiomer, the solvents were evaporated and the residue was purified by reverse phase HPLC (1-99% acetonitrile/5 mM aqueous HCl over 15 min) to give as yellow solids: diastereomer A, SFC peak 1, 4-[6-[cyclobutyl(methyl)amino]pyrazin-2-yl]-14-(2,6-dimethylphenyl)-8-isobutyl-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one (7.1 mg, 53%). ¹H NMR (400 MHz, Chloroform-d) δ 8.68-8.63 (m, 1H), 7.92 (d, J=7.9 Hz, 1H), 7.87 (s, 1H), 7.84 (s, 1H), 7.80 (dt, J=7.7, 1.4 Hz, 1H), 7.59 (t, J=7.8 Hz, 1H), 7.22 (t, J=7.6 Hz, 1H), 7.08 (d, J=7.6 Hz, 2H), 6.27 (s, 1H), 5.71 (dd, J=11.7, 5.4 Hz, 1H), 5.58-5.46 (m, 1H), 4.89 (t, J=11.8 Hz, 1H), 4.49 (p, J=8.4 Hz, 1H), 4.40 (dt, J=11.2, 5.0 Hz, 1H), 3.22 (s, 3H), 2.39-2.28 (m, 2H), 2.28-2.19 (m, 2H), 2.16-2.08 (m, 2H), 2.05 (s, 6H), 1.97-1.88 (m, 2H), 1.87-1.79 (m, 1H), 1.80-1.69 (m, 3H), 1.68-1.60 (m, 1H), 1.27-1.10 (m, 2H), 0.83-0.71 (m, 1H), 0.68-0.60 (m, 6H). ESI-MS m/z calc. 709.341, found 710.81 (M+1)⁺; Retention time: 2.08 minutes; and diastereomer B, SFC peak 2, 4-[6-[cyclobutyl(methyl) amino]pyrazin-2-yl]-14-(2,6-dimethylphenyl)-8-isobutyl-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one (8.5 mg, 63%), ¹H NMR (400 MHz, Chloroform-d) δ 8.66 (s, 1H), 7.93 (d, J=7.9 Hz, 1H), 7.87 (s, 1H), 7.83 (s, 1H), 7.80 (d, J=7.3 Hz, 1H), 7.59 (t, J=7.8 Hz, 1H), 7.22 (t, J=7.6 Hz, 1H), 7.08 (d, J=7.6 Hz, 2H), 6.28 (s, 1H), 5.71 (dd, J 11.6, 5.4 Hz, 1H), 5.57-5.46 (m, 1H), 4.89 (t, J=11.8 Hz, 1H), 4.49 (p, J=8.4 Hz, 1H), 4.44-4.32 (m, 1H), 3.22 (s, 3H), 2.38-2.29 (m, 2H), 2.28-2.19 (m, 2H), 2.17-2.08 (m, 2H), 2.05 (s, 6H), 1.97-1.88 (m, 2H), 1.88-1.81 (m, 1H), 1.80-1.69 (m, 3H), 1.68-1.58 (m, 1H), 1.25-1.12 (m, 2H), 0.84-0.72 (m, 1H), 0.70-0.57 (m, 6H). ESI-MS m/z calc. 709.341, found 710.75 (M+1)⁺; Retention time: 2.08 minutes; LC method A.

### Example 152: Preparation of Compound 402 and Compound 403

### Step 1: 4-[6-[Cyclobutyl(methyl)amino]pyrazin-2-yl]-14-(2,6-dimethylphenyl)-8-isobutyl-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one, isomer C, SFC peak 1 (Compound 402), and 4-[6-[cyclobutyl(methyl) amino]pyrazin-2-yl]-14-(2,6-dimethylphenyl)-8-isobutyl-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one, isomer C, SFC peak 2 (Compound 403)

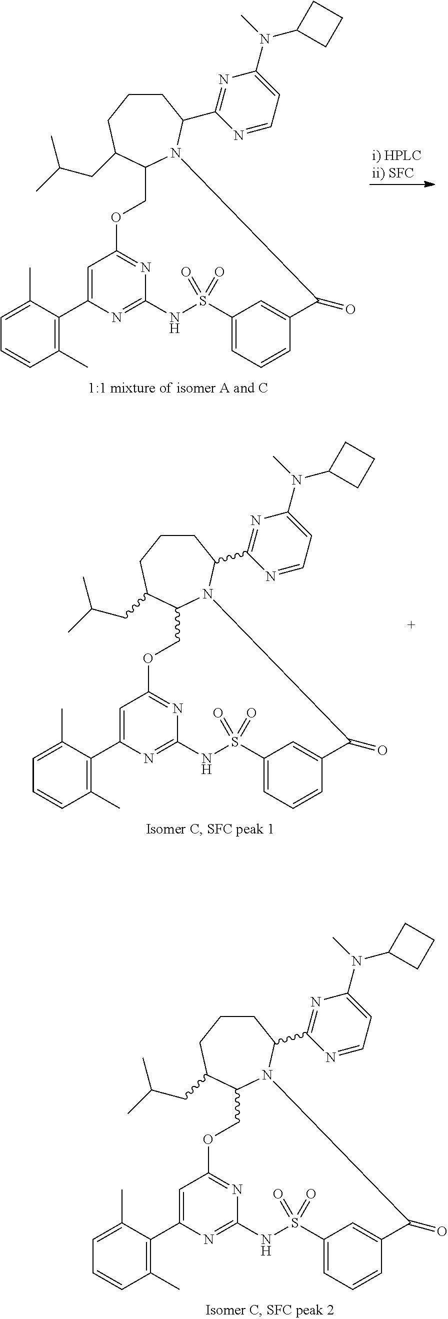

4-[6-[Cyclobutyl(methyl)amino]pyrazin-2-yl]-14-(2,6-dimethylphenyl)-8-isobutyl-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one (57 mg, 0.08029 mmol) (1:1 mixture of diastereomer A and C) was dissolved in DMSO (1 mL) and subjected to reverse phase HPLC (1-99% acetonitrile/5 mM aqueous HCl over 15 min, mixed fractions purified a second time) to give two racemic diastereomers: first to elute, more polar diastereomer C 4-[6-[cyclobutyl(methyl)amino]pyrazin-2-yl]-14-(2,6-dimethylphenyl)-8-isobutyl-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one (12.3 mg, 43%), ESI-MS m/z calc. 709.341, found 710.75 (M+1)⁺; Retention time: 1.96 minutes and second to elute, less polar diastereomer A 4-[6-[cyclobutyl(methyl)amino]pyrazin-2-yl]-14-(2,6-dimethylphenyl)-8-isobutyl-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one (14.7 mg, 52%), ESI-MS m/z calc. 709.341, found 710.81 (M+1)⁺; Retention time: 2.03 minutes. The more polar diastereomer C was subjected to chiral SFC using a ChiralPak AS column (250×21.2 mm; 5 um) at 40° C. The mobile phase was 44% MeOH (20 mM NH₃), at a 70 mL/min flow. Concentration of the sample was 18 mg/mL in methanol (no modifier), injection volume 200 μL with an outlet pressure of 235 bar, detection wavelength of 210 nm. For each resulting enantiomer, the solvents were evaporated and the residue was purified by reverse phase HPLC (1-99% acetonitrile/5 mM aqueous HCl over 15 min) to give diastereomer C, SFC peak 1, 4-[6-[cyclobutyl(methyl)amino]pyrazin-2-yl]-14-(2,6-dimethylphenyl)-8-isobutyl-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one (0.5 mg, 3%). ESI-MS m/z calc. 709.341, found 710.81 (M+1)⁺; Retention time: 1.98 minutes and diastereomer C, SFC peak 2, 4-[6-[cyclobutyl(methyl)amino]pyrazin-2-yl]-14-(2,6-dimethylphenyl)-8-isobutyl-18,18-dioxo-11-oxa-18λ⁶-thia-3,15,17,24-tetrazatetracyclo[17.3.1.112,16.03,9]tetracosa-1(22),12,14,16(24),19(23),20-hexaen-2-one (0.2 mg, 1%). ESI-MS m/z calc. 709.341, found 710.59 (M+1)⁺; Retention time: 1.98 minutes; LC method A.

### Example 153: Preparation of Compound 404 and Compound 405

### Step 1: tert-Butyl N-[(1R)-6-benzyloxy-2-hydroxy-1-isobutyl-hexyl]carbamate

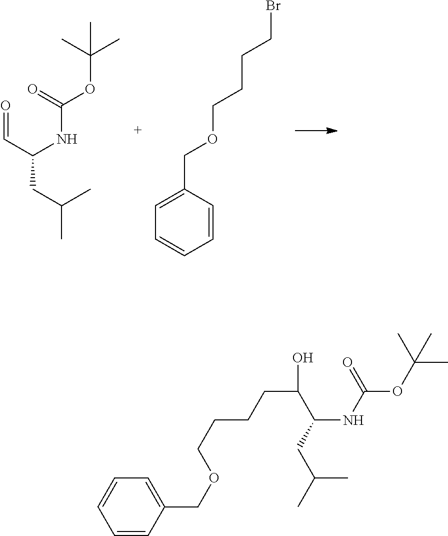 [SMILES: CC(C)C[C@H](C=O)NC(=O)OC(C)(C)C ; BrCCCCOCc1ccccc1 ; CC(C)C[C@@H](NC(=O)OC(C)(C)C)C(O)CCCCOCc1ccccc1]

Magnesium (4.5 g, 185.15 mmol) was placed in a dry flask under argon balloon. A tiny iodine crystal was added. The flask was briefly heated with a heating gun until liberation of purple iodine vapor was visible. The content was cooled to RT. Anhydrous THE (100 mL) was added, followed by few drops of 4-bromobutoxymethylbenzene (12.750 g, 10 mL, 52.439 mmol) in anhydrous THE (100 mL). 1,2-dibromoethane (868.00 mg, 0.4 mL, 4.6204 mmol) was then added. The mixture was stirred with periodical gentle heating until a clear mixture was observed. The remaining bromide was added dropwise. After complete addition, the mixture was placed in a 50° C. oil bath and stirred under an argon for 1 h. The reaction was then cooled to 0° C. and tert-butyl N-[(1R)-1-formyl-3-methyl-butyl]carbamate (10 g, 41.804 mmol) in anhydrous THE (100 mL) was added over 20 min. The reaction was stirred at 0° C. for 1 h. The reaction was then quenched with saturated ammonium chloride (150 ml), aqueous solution was extracted with Ethyl acetate (3×200 ml). The combined organic solution was then washed with brine (150 ml), dried over anhydrous sodium sulfate, filtered and concentrated. The crude product was purified by silica gel chromatography (loaded with DCM in 330 g cartridge using 0-40% ethyl acetate in hexanes as eluent) to provide tert-butyl N-[(1R)-6-benzyloxy-2-hydroxy-1-isobutyl-hexyl]carbamate (4.6 g, 28%) as light yellow oil. ESI-MS m/z calc. 379.27225, found 380.3 (M+1)⁺; Retention time: 6.0 minutes; LC method S.

### Step 2: tert-Butyl N-[(1R)-6-benzyloxy-2-[tert-butyl(dimethyl)silyl]oxy-1-isobutyl-hexyl]carbamate

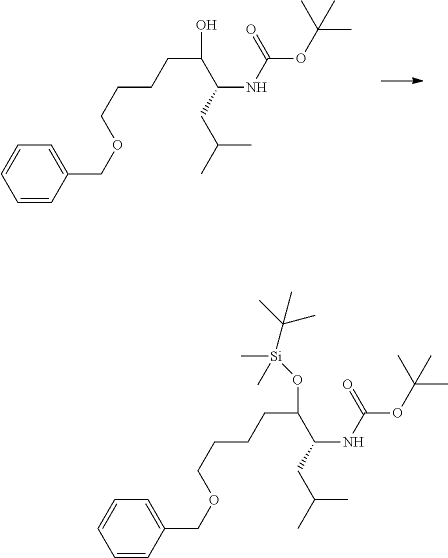 [SMILES: CC(C)C[C@@H](NC(=O)OC(C)(C)C)C(O)CCCCOCc1ccccc1 ; CC(C)C[C@@H](NC(=O)OC(C)(C)C)C(CCCCOCc1ccccc1)O[Si](C)(C)C(C)(C)C]

(1-Methyl-1-silyl-ethyl)N-[(1R)-6-benzyloxy-2-hydroxy-1-isobutyl-hexyl]carbamate (11 g, 26.415 mmol) was dissolved in anhydrous DCM (50 mL) under argon and the solution was cooled to 0° C. imidazole (6.8 g, 99.886 mmol) was added to the reaction and stirred for 5 min. TBSCl (10.9 g, 72.319 mmol) was then added and followed by DMAP (1.1 g, 9.0040 mmol). The reaction was then warmed up to room temperature after 5 min. Then the reaction mixture was then heated at 53° C. for 2 h. The reaction was then cooled to room temperature, diluted with DCM (100 mL) quenched with saturated ammonium chloride solution (150 mL). The aqueous layer was extracted with DCM (3×100 mL), washed with brine (100 mL) dried over anhydrous sodium sulfate, filtered and the solvent was removed under reduced pressure. The crude product was purified by silica gel chromatography (loaded with DCM in 330 g cartridge using 0-30% diethyl ether in hexanes as eluent) to provide tert-butyl N-[(1R)-6-benzyloxy-2-[tert-butyl(dimethyl)silyl]oxy-1-isobutyl-hexyl]carbamate (7.9 g, 59%) as light yellow oil. ¹H NMR (500 MHz, Chloroform-d) δ 7.40-7.31 (m, 4H), 7.29-7.27 (m, 1H), 4.59-4.54 (m, 1H), 4.54-4.47 (m, 2H), 3.73-3.65 (m, 1H), 3.62-3.56 (m, 1H), 3.51-3.43 (m, 2H), 1.67-1.58 (m, 4H), 1.44 (s, 9H), 1.32-1.23 (m, 2H), 0.99-0.82 (m, 18H), 0.11-0.01 (m, 6H). ESI-MS m/z calc. 493.35873, found 494.5 (M+1)⁺; Retention time: 9.5 minutes; LC method S.

### Step 3: tert-Butyl N-[(1R)-2-[tert-butyl(dimethyl)silyl]oxy-6-hydroxy-1-isobutyl-hexyl]carbamate

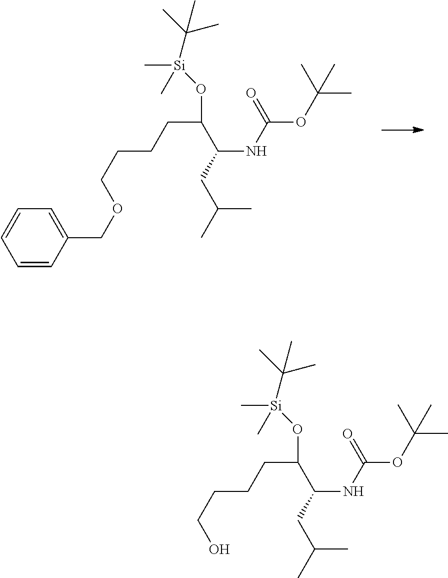 [SMILES: CC(C)C[C@@H](NC(=O)OC(C)(C)C)C(CCCCOCc1ccccc1)O[Si](C)(C)C(C)(C)C ; CC(C)C[C@@H](NC(=O)OC(C)(C)C)C(CCCCO)O[Si](C)(C)C(C)(C)C]

To a solution of tert-butyl N-[(1R)-6-benzyloxy-2-[tert-butyl(dimethyl) silyl]oxy-1-isobutyl-hexyl]carbamate (7.9 g, 15.999 mmol) in Ethanol (160 mL) was cooled to 0° C. and added Palladium on carbon (1.709 g, 10% w/w, 1.6059 mmol) under N2 gas. A H2-gas filled balloon was bubbled through the mixture at 0° C. for 3 h. The reaction was purged with nitrogen gas, warmed to room temperature, filtered through a pad of celite and washed with methanol (3×100 mL). The solvent was removed in vacuo, and the residual EtOH was removed under high vacuum at 45° C., to give tert-butyl N-[(1R)-2-[tert-butyl(dimethyl)silyl]oxy-6-hydroxy-1-isobutyl-hexyl]carbamate (5.30 g, 81%) as colorless oil as diastereomers ESI-MS m/z calc. 403.3118, found 404.5 (M+1)⁺; Retention time: 8.0 minutes; LC method S.

### Step 4: tert-Butyl N-[(1R)-2-[tert-butyl(dimethyl)silyl]oxy-1-isobutyl-6-oxo-hexyl]arbamate

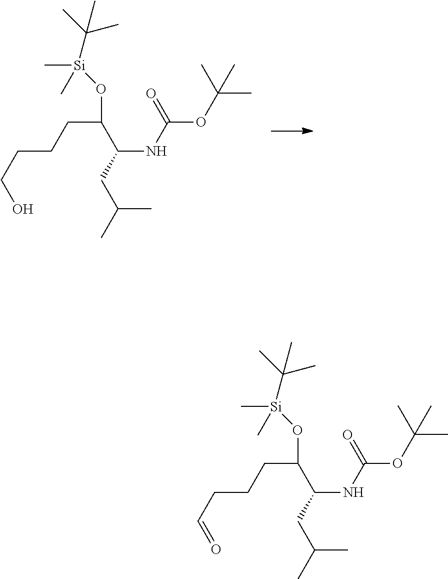 [SMILES: CC(C)C[C@@H](NC(=O)OC(C)(C)C)C(CCCCO)O[Si](C)(C)C(C)(C)C ; CC(C)C[C@@H](NC(=O)OC(C)(C)C)C(CCCC=O)O[Si](C)(C)C(C)(C)C]

tert-Butyl N-[(1R)-2-[tert-butyl(dimethyl)silyl]oxy-6-hydroxy-1-isobutyl-hexyl]carbamate (3.8 g, 8.9429 mmol) was dissolve in anhydrous DMSO (35 mL) at room temperature. IBX (4.3 g, 14.896 mmol) was added to the reaction mixture. The reaction was then stirred at room temperature for 2 h. The reaction was diluted with water (50 ml) and EtOAc (100 ml). The white solid (IBX byproducts) was filtered through a sintered funnel washing thoroughly with ethyl acetate. The aqueous solution was extracted with EtOAc (3×100 ml). The combined organic solution was washed with brine (100 ml), dried over anhydrous sodium sulfate, filtered and the solvent was removed under reduced pressure. The crude product was purified by flash chromatography (Combiflash, loaded with DCM in 40 g cartridge using 0-30% diethylether in hexanes (1% Et₃N as modifier) as eluent) to provide tert-butyl N-[(1R)-2-[tert-butyl(dimethyl)silyl]oxy-1-isobutyl-6-oxo-hexyl]carbamate (2.4 g, 66%) as colorless oil. ESI-MS m/z calc. 401.29614, found 402.2 (M+1)⁺; Retention time: 4.59 minutes; LC method T.

### Step 5: tert-Butyl N-[(1R)-2-[tert-butyl(dimethyl)silyl]oxy-6-hydroxy-1-isobutyl-6-(5-isopropoxypyrimidin-2-yl)hexyl]carbamate

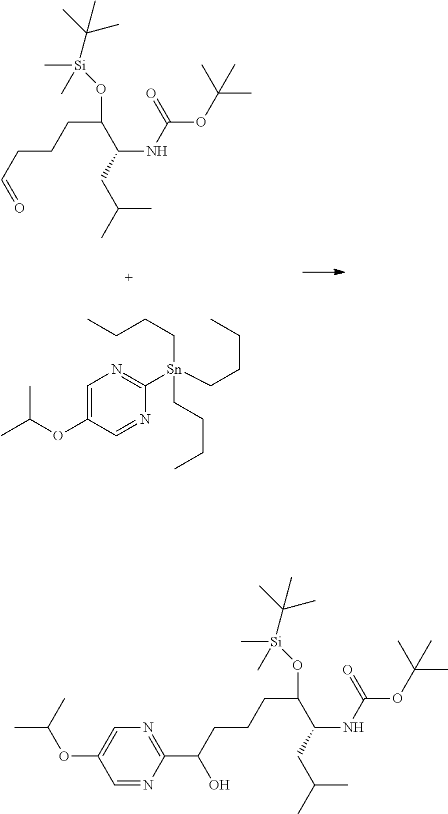 [SMILES: CC(C)C[C@@H](NC(=O)OC(C)(C)C)C(CCCC=O)O[Si](C)(C)C(C)(C)C ; CCC[CH2][Sn]([CH2]CCC)([CH2]CCC)[c]1ncc(OC(C)C)cn1 ; CC(C)C[C@@H](NC(=O)OC(C)(C)C)C(CCCC(O)c1ncc(OC(C)C)cn1)O[Si](C)(C)C(C)(C)C]

A solution of n-BuLi in hexanes (2.4 mL of 2.5 M, 6.00 mmol) at −90° C. to −95° C. was added tributyl-(5-isopropoxypyrimidin-2-yl)stannane (2.32 g, 2 mL, 5.4306 mmol) in THE (26.6 mL) dropwise over 26 minutes and the reaction was stirred at −90° C. to −95° C. for 45 minutes. To the pyrimidyl-lithium solution was added a solution of tert-butyl N-[(1R)-2-[tert-butyl(dimethyl)silyl]oxy-1-isobutyl-6-oxo-hexyl]carbamate (2 g, 4.9794 mmol) in THE (6.6 mL) at −95° C. dropwise over 5 minutes then the reaction was stirred at −95° C. for 15 minutes and allowed to warm to −20° C. over 2 h, quenched with saturated aqueous NH₄Cl (20 mL), diluted with water (10 mL), extracted with EtOAc (3×35 mL), washed with brine, dried over Na₂SO₄, filtered and concentrated in vacuo. The residue was purified by flash chromatography (Combiflash, loaded onto a 80 g SiO₂ cartridge with benzene, and eluted with 0-40% EtOAc in Hexanes over a 35 min gradient) to provide tert-butyl N-[(1R)-2-[tert-butyl(dimethyl)silyl]oxy-6-hydroxy-1-isobutyl-6-(5-isopropoxypyrimidin-2-yl)hexyl]carbamate (1.22 g, 45%) as a yellow oil, as diastereomers ESI-MS m/z calc. 539.3754, found 540.7 (M+1)⁺; Retention time: 8.08 minutes; LC method S.

### Step 6: [(6R)-6-(tert-Butoxycarbonylamino)-5-[tert-butyl(dimethyl)silyl]oxy-1-(5-isopropoxypyrimidin-2-yl)-8-methyl-nonyl]methanesulfonate

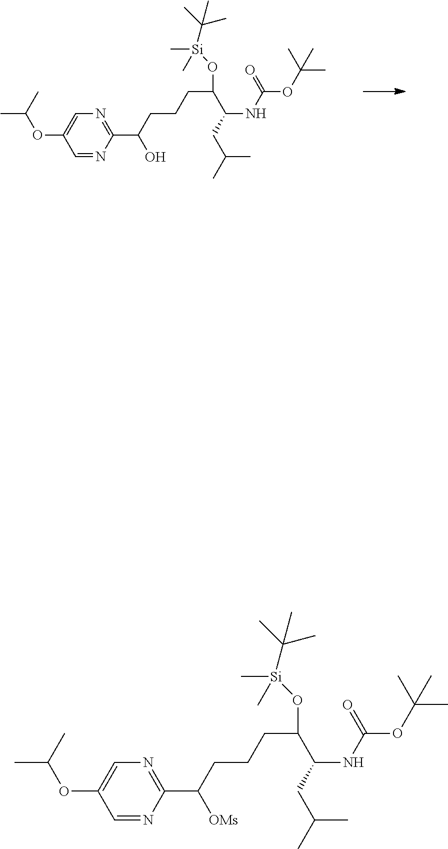

A solution of tert-butyl N-[(1R)-2-[tert-butyl(dimethyl)silyl]oxy-6-hydroxy-1-isobutyl-6-(5-isopropoxypyrimidin-2-yl)hexyl]carbamate (670 mg, 1.2399 mmol) in DCM (4.25 mL) was cooled to 0° C. then added Et3N (254.10 mg, 0.350 mL, 2.5111 mmol) followed by MsCl (148.00 mg, 0.10 mL, 1.2920 mmol). The reaction was warmed to room temperature and stirred 30 minutes then diluted with DCM (30 mL) washed with saturated aqueous NH₄Cl (20 mL), then extracted the aqueous with DCM (2×30 mL). The combined organics were washed with water (20 mL), dried over MgSO4, filtered and concentrated in vacuo to provide [(6R)-6-(tert-butoxycarbonylamino)-5-[tert-butyl(dimethyl)silyl]oxy-1-(5-isopropoxypyrimidin-2-yl)-8-methyl-nonyl]methanesulfonate (766 mg, 95%) as a pale yellow oil as diastereomers ESI-MS m/z calc. 617.353, found 618.7 (M+1)⁺; Retention time: 8.83 minutes, 8.94 minutes. LC method S.

### Step 7: tert-Butyl-[(2R)-2-isobutyl-7-(5-isopropoxypyrimidin-2-yl)azepan-3-yl]oxy-dimethyl-silane

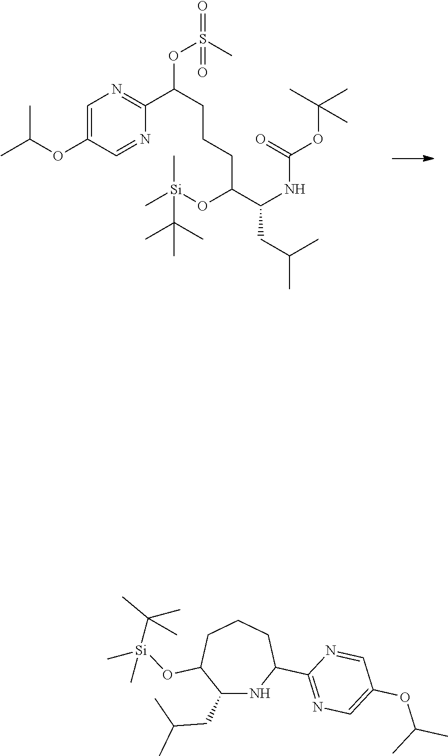

To a vial containing [(6R)-6-(tert-butoxycarbonylamino)-5-[tert-butyl(dimethyl)silyl]oxy-1-(5-isopropoxypyrimidin-2-yl)-8-methyl-nonyl]methanesulfonate (766.15 mg, 1.2387 mmol) was added TFA (5.0320 g, 3.4 mL, 44.131 mmol) at 0° C. and the mixture was stirred 5 minutes then warmed to room temperature and stirred for 10 minutes. The reaction was concentrated in vacuo to provide [(6R)-6-amino-5-[tert-butyl(dimethyl)silyl]oxy-1-(5-isopropoxypyrimidin-2-yl)-8-methyl-nonyl]methanesulfonate (trifluoroacetate salt) (782 mg, 100%) as a pale yellow oil, as diastereomers ESI-MS m/z calc. 517.3006, found 518.7 (M+1)⁺; Retention time: 5.1 minutes and 5.25 minutes. The oil residue was mixed with anhydrous DMF (11.2 mL) and Cs₂CO₃ (2.3 g, 7.0592 mmol) and heated for 14 h at 60° C. The reaction was cooled to room temperature and quenched with saturated NH₄Cl (30 mL), then extracted with EtOAc (3×30 mL). The combined organic layers were washed with a mixture of 1M NaOH (30 mL) and brine (60 mL), dried over anhydrous Na₂SO₄, filtered and concentrated in vacuo (436 mg crude). The residue was purified by flash chromatography (loaded onto a 12 g SiO₂ cartridge with benzene, and eluted with 0-5% MeOH containing 0.17 M NH₃, in DCM over a 30 CV gradient) to provide tert-butyl-[(2R)-2-isobutyl-7-(5-isopropoxypyrimidin-2-yl)azepan-3-yl]oxy-dimethyl-silane (330 mg, 51%) as a yellow oil as diastereomers ESI-MS m/z calc. 421.3125, found 422.6 (M+1)⁺; Retention time: 6.26 minutes and 6.54 minutes. LC method S.

### Step 8: (2R)-2-Isobutyl-7-(5-isopropoxypyrimidin-2-yl)azepan-3-ol

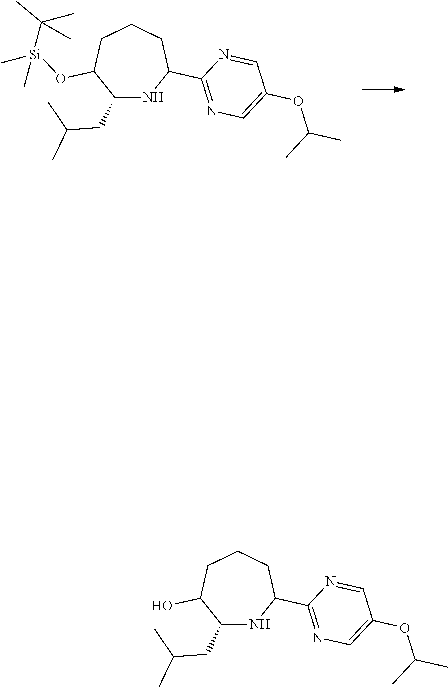

To a solution of tert-butyl-[(2R)-2-isobutyl-7-(5-isopropoxypyrimidin-2-yl)azepan-3-yl]oxy-dimethyl-silane (330 mg, 0.7826 mmol) in MeOH (11.8 mL) was added HCl (9.4 mL of 3 M, 28.200 mmol) and heated in a 65° C. oil bath for 18 h. The reaction was concentrated in vacuo (50° C.) to a light orange foam and the residue was diluted with EtOAc (20 mL) and added 2 M NaOH (10 mL) and solid NaCl until the aqueous was saturated. The aqueous was extracted with EtOAc (5×20 mL) and the organics were dried over Na₂SO₄, filtered and concentrated to provide crude (2R)-2-isobutyl-7-(5-isopropoxypyrimidin-2-yl)azepan-3-ol (255 mg, 90%) as a mixture of diastereomers ESI-MS m/z calc. 307.226, found 308.6 (M+1)⁺; Retention time: 3.51 minutes, 3.18 minutes, 2.99 minutes, 2.66 minutes. LC method S.

### Step 9: (2R)-2-Isobutyl-7-(5-isopropoxypyrimidin-2-yl)azepan-3-ol, isomer 1, and (2R)-2-isobutyl-7-(5-isopropoxypyrimidin-2-yl)azepan-3-ol, isomer 2, and (2R)-2-isobutyl-7-(5-isopropoxypyrimidin-2-yl)azepan-3-ol, isomer 3

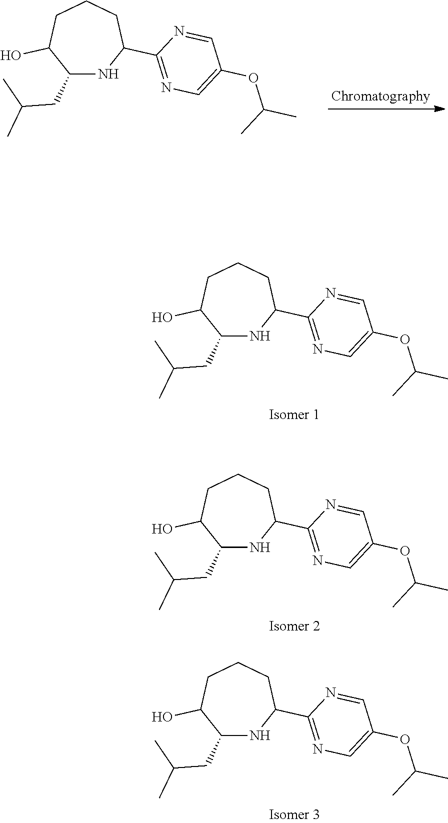

An isomeric mixture of (2R)-2-Isobutyl-7-(5-isopropoxypyrimidin-2-yl)azepan-3-ol (507 mg) was purified by flash chromatography (loaded onto a 24 g SiO₂ cartridge with DCM, and eluted with 0-5% MeOH in DCM over a 60 min gradient) to provide, separately, (2R)-2-isobutyl-7-(5-isopropoxypyrimidin-2-yl)azepan-3-ol, isomer 1 (107 mg, 25%) as a colorless oil, ESI-MS m/z calc. 307.226, found 308.4 (M+1)⁺; Retention time: 3.07 minutes, ¹H NMR (500 MHz, Chloroform-d) δ 8.34 (s, 2H), 4.64-4.52 (m, 1H), 4.25 (t, J=5.7 Hz, 1H), 3.70-3.65 (m, 1H), 2.94 (dd, J=8.4, 5.8 Hz, 1H), 2.19-2.12 (m, 1H), 2.12-2.04 (m, 1H), 1.88 (ddt, J 13.3,6.5, 3.3, 3.3 Hz, 1H), 1.82-1.67 (m, 2H), 1.62-1.54 (m, 1H), 1.53-1.41 (m, 3H), 1.39-1.34 (m, 3H), 0.98-0.80 (m, 6H); (2R)-2-isobutyl-7-(5-isopropoxypyrimidin-2-yl)azepan-3-ol, isomer 2 (30 mg, 7%) as a colorless oil, ESI-MS m/z calc. 307.226, found 308.4 (M+1)⁺; Retention time: 3.19 minutes, ¹H NMR (500 MHz, Chloroform-d) δ 8.32 (s, 2H), 4.59 (p, J=6.1 Hz, 1H), 4.19 (dd, J=7.8, 5.7 Hz, 1H), 3.60 (td, J=6.5, 6.5, 3.1 Hz, 1H), 3.04 (ddd, J=10.0, 6.2, 4.2 Hz, 1H), 2.93 (s, 2H), 2.17-2.08 (m, 1H), 2.08-1.99 (m, 1H), 1.93-1.78 (m, 3H), 1.65-1.55 (m, 2H), 1.54-1.47 (m, 1H), 1.38-1.35 (m, 6H), 1.36-1.32 (m, 1H), 0.89 (d, J 6.6 Hz, 3H), 0.77 (d, J=6.5 Hz, 3H); (2R)-2-isobutyl-7-(5-isopropoxypyrimidin-2-yl)azepan-3-ol, isomer 3 (7 mg, 2%) as a colorless oil, ESI-MS m/z calc. 307.226, found 308.4 (M+1)⁺; Retention time: 3.57 minutes; ¹H NMR (500 MHz, Chloroform-d) δ 8.32 (s, 1H), 4.61 (hept, J=6.1 Hz, 1H), 4.14 (dd, J=11.5, 6.7 Hz, 1H), 3.56 (t, J=3.1, 3.1 Hz, 1H), 3.12 (dd, J=7.9, 6.1 Hz, 1H), 2.86-2.43 (m, 2H), 2.34-2.22 (m, 1H), 2.00-1.89 (m, 1H), 1.88-1.79 (m, 1H), 1.73-1.63 (m, 1H), 1.40-1.35 (m, 6H), 1.35-1.33 (m, 1H), 1.34-1.26 (m, 2H), 0.98-0.84 (m, 1H), 0.79 (d, J=6.5 Hz, 3H), 0.67 (d, J=6.5 Hz, 3H); and (2R)-2-isobutyl-7-(5-isopropoxypyrimidin-2-yl)azepan-3-ol (mixture of isomer 1 and 3, 70 mg, 16%) as a colorless oil, ESI-MS m/z calc. 307.226, found 308.2 (M+1)⁺; Retention time: 3.02 minutes and 3.53 minutes; LC method S.

### Step 10: (21R)-12-(2,6-Dimethylphenyl)-21-isobutyl-20-(5-isopropoxypyrimidin-2-yl)-8,8-dioxo-15-oxa-8λ⁶-thia-1,9,11,22-tetrazatetracyclo[14.4.1.13,7.110,14]tricosa-3,5,7(23),10(22),11,13-hexaen-2-one, isomer 1

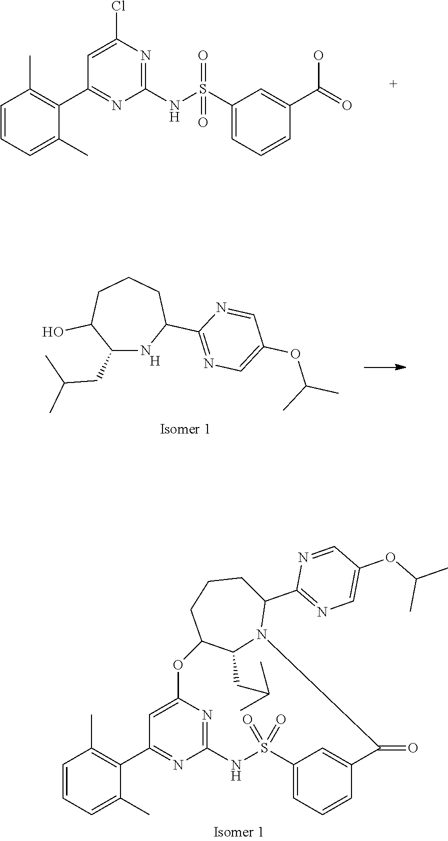

To a solution of (2R)-2-isobutyl-7-(5-isopropoxypyrimidin-2-yl)azepan-3-ol, isomer 1 (79 mg, 0.2570 mmol) in THE (1.58 mL) was added sodium t-butoxide (124 mg, 1.2903 mmol) at 0° C. The reaction was warmed to room temperature and stirred 10 minutes until a solution formed. Then the reaction was cooled to 0° C., stirred 5 minutes and 3-[[4-chloro-6-(2,6-dimethylphenyl)pyrimidin-2-yl]sulfamoyl]benzoic acid (128 mg, 0.3063 mmol) was added. The reaction was stirred for 15 minutes then warmed to room temperature and stirred for 4 h. The reaction was quenched with aqueous 1M HCl (2 mL), extracted with EtOAc (3×4 mL), washed with brine (4 mL) dried over Na₂SO₄, filtered and concentrated in vacuo to provide crude 3-[[4-(2,6-dimethylphenyl)-6-[(2R)-2-isobutyl-7-(5-isopropoxypyrimidin-2-yl)azepan-3-yl]oxy-pyrimidin-2-yl]sulfamoyl]benzoic acid (hydrochloride salt) (186 mg, 100%) as off-white solids ESI-MS m/z calc. 688.3043, found 689.7 (M+1)⁺; Retention time: 4.38 minutes. The residue was diluted in DMF (15.8 mL) and to the solution was added NMM (147.20 mg, 0.160 mL, 1.4553 mmol) then CDMT (95 mg, 0.5411 mmol) at room temperature and the mixture was stirred for 24 h. The reaction was quenched with 10% citric acid (40 mL) and extracted with EtOAc (3×40 mL). The combined organic layers were washed with brine (3×60 mL), dried over anhydrous Na₂SO₄, filtered and concentrated in vacuo. The residue was purified by flash chromatography (loaded onto a 12 g SiO₂ cartridge with benzene, and eluted with 20-70% EtOAc in Hexanes over a 40 min gradient) to provide (21R)-12-(2,6-dimethylphenyl)-21-isobutyl-20-(5-isopropoxypyrimidin-2-yl)-8,8-dioxo-15-oxa-8λ⁶-thia-1,9,11,22-tetrazatetracyclo[14.4.1.13,7.110,14]tricosa-3,5,7(23),10(22),11,13-hexaen-2-one, isomer 1 (41 mg, 23%) as a white solid. ¹H NMR (500 MHz, Chloroform-d) δ 8.80 (s, 1H), 8.63 (s, 1H), 8.31 (s, 2H), 8.04-7.95 (m, 1H), 7.65 (dt, J=7.6, 1.4, 1.4 Hz, 1H), 7.57 (t, J=7.8 Hz, 1H), 7.20 (t, J=7.6 Hz, 1H), 7.05 (d, J=7.7 Hz, 2H), 6.08 (s, 1H), 5.67 (dd, J=13.1, 6.3 Hz, 1H), 5.48 (dt, J 10.7, 4.9, 4.9 Hz, 1H), 4.60 (p, J=6.1 Hz, 1H), 4.15-4.08 (m, 1H), 2.87-2.78 (m, 1H), 2.25-2.16 (m, 1H), 2.12-2.04 (m, 1H), 2.01-1.89 (m, 7H), 1.87-1.74 (m, 2H), 1.73-1.62 (m, 2H), 1.41-1.35 (m, 6H), 1.30-1.22 (m, 1H), 0.63 (d, J=6.6 Hz, 3H), 0.06 (d, J=6.6 Hz, 3H). ESI-MS m/z calc. 670.29376, found 671.8 (M+1)⁺; Retention time: 3.13 minutes; LC method W.

### Step 11: 12-(2,6-Dimethylphenyl)-21-isobutyl-20-(5-isopropoxypyrimidin-2-yl)-8,8-dioxo-15-oxa-8λ⁶-thia-1,9,11,22-tetrazatetracyclo[14.4.1.13,7.110,14]tricosa-3,5,7(23),10(22),11,13-hexaen-2-one, isomer 1, SFC peak 1 (Compound 405), and 12-(2,6-dimethylphenyl)-21-isobutyl-20-(5-isopropoxypyrimidin-2-yl)-8,8-dioxo-15-oxa-8λ⁶-thia-1,9,11,22-tetrazatetracyclo[14.4.1.13,7.110,14]tricosa-3,5,7(23),10(22),11,13-hexaen-2-one, isomer 1, SFC peak 2 (Compound 404)

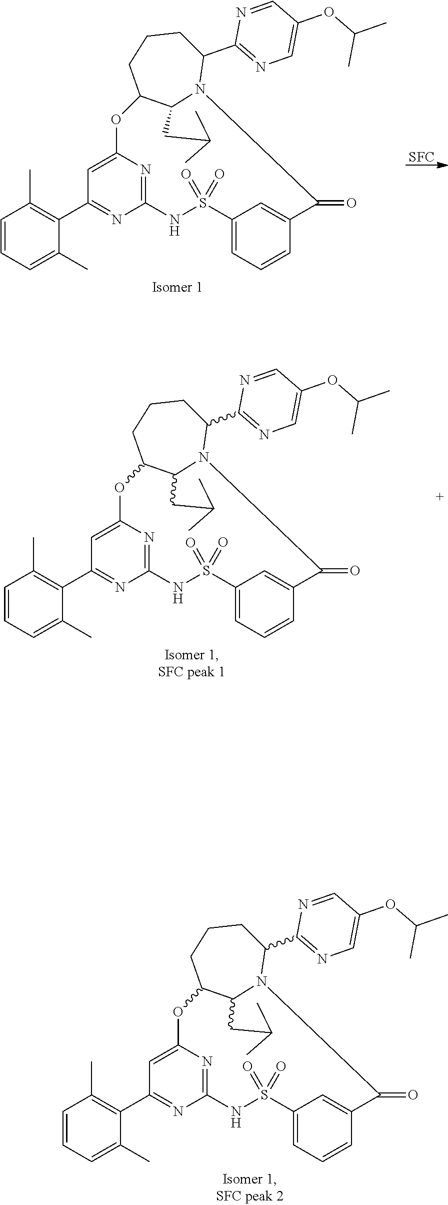 [SMILES: Cc1cccc(C)c1-c1cc2nc(n1)NS(=O)(=O)c1cccc(c1)C(=O)N1C(c3ncc(OC(C)C)cn3)CCCC(O2)[C@H]1CC(C)C ; "SFC" ; Cc1cccc(C)c1-c1cc2nc(n1)NS(=O)(=O)c1cccc(c1)C(=O)N1C(c3ncc(OC(C)C)cn3)CCCC(O2)C1CC(C)C ; Cc1cccc(C)c1-c1cc2nc(n1)NS(=O)(=O)c1cccc(c1)C(=O)N1C(c3ncc(OC(C)C)cn3)CCCC(O2)C1CC(C)C]

(21R)-12-(2,6-Dimethylphenyl)-21-isobutyl-20-(5-isopropoxypyrimidin-2-yl)-8,8-dioxo-15-oxa-8Δ⁶-thia-1,9,11,22-tetrazatetracyclo[14.4.1.13,7.110,14]tricosa-3,5,7(23),10(22),11,13-hexaen-2-one (41 mg, 0.06112 mmol)(isomer 1, racemic) was subjected to chiral SFC separation using a Phenomenex Lux-4 column (250×21.2 mm, 5 μM) at 50° C. The mobile phase was 44% MeOH (20 mM NH₃) at a 70 mL/min flow in isocratic mode. Concentration of the sample was 27.3 mg/mL in a mixture of methanol and DMSO (76:24, v:v), injection volume 700 μL with an outlet pressure of 175 bar, detection wavelength of 210 nm to give two isomers. For each isomer, the solvents were evaporated and the residue purified by reverse phase HPLC (1-99% acetonitrile/5 mM aqueous HCl over 15 min) to give as off-white solids: isomer 1, SFC peak 1, 12-(2,6-dimethylphenyl)-21-isobutyl-20-(5-isopropoxypyrimidin-2-yl)-8,8-dioxo-15-oxa-8λ⁶-thia-1,9,11,22-tetrazatetracyclo[14.4.1.13,7.110,14]tricosa-3,5,7(23),10(22),11,13-hexaen-2-one (10.8 mg, 53%), ¹H NMR (400 MHz, Chloroform-d) δ 9.04 (broad s, 1H), 8.66-8.58 (m, 1H), 8.31 (s, 2H), 8.06 (dt, J=7.8, 1.5 Hz, 1H), 7.68-7.62 (m, 1H), 7.58 (t, J=7.7 Hz, 1H), 7.19 (t, J=7.6 Hz, 1H), 7.04 (d, J=7.6 Hz, 2H), 6.07 (s, 1H), 5.68 (dd, J=13.1, 6.3 Hz, 1H), 5.53-5.39 (m, 1H), 4.60 (hept, J=5.9 Hz, 1H), 4.12 (dd, J=8.6, 4.8 Hz, 1H), 2.90-2.76 (m, 1H), 2.27-2.17 (m, 1H), 2.14-2.02 (m, 1H), 2.02-1.91 (m, 7H), 1.89-1.77 (m, 2H), 1.74-1.63 (m, 2H), 1.44-1.35 (m, 6H), 1.25 (dd, J=14.0, 10.5 Hz, 1H), 0.63 (d, J=6.6 Hz, 3H), 0.06 (d, J=6.5 Hz, 3H). ESI-MS m/z calc. 670.29376, found 671.74 (M+1)⁺; Retention time: 2.13 minutes, and isomer 1, SFC peak 2, 12-(2,6-dimethylphenyl)-21-isobutyl-20-(5-isopropoxypyrimidin-2-yl)-8,8-dioxo-15-oxa-8λ⁶-thia-1,9,11,22-tetrazatetracyclo[14.4.1.13,7.110,14]tricosa-3,5,7(23),10(22),11,13-hexaen-2-one (10.4 mg, 51%), ¹H NMR (400 MHz, Chloroform-d) δ 9.04 (broad s, 1H), 8.68-8.56 (m, 1H), 8.31 (s, 2H), 8.06 (dt, J=7.9, 1.6 Hz, 1H), 7.66 (dt, J=7.7, 1.5 Hz, 1H), 7.58 (t, J=7.7 Hz, 1H), 7.19 (t, J=7.6 Hz, 1H), 7.04 (d, J=7.6 Hz, 2H), 6.07 (s, 1H), 5.68 (dd, J=13.1, 6.4 Hz, 1H), 5.48 (dt, J=10.9, 5.1 Hz, 1H), 4.60 (hept, J=6.6 Hz, 1H), 4.12 (dd, J=8.7, 4.7 Hz, 1H), 2.83 (dt, J=14.8, 7.3 Hz, 1H), 2.30-2.16 (m, 1H), 2.14-2.02 (m, 1H), 2.00-1.88 (m, 7H), 1.88-1.75 (m, 2H), 1.71-1.60 (m, 2H), 1.42-1.33 (m, 6H), 1.25 (dd, J=13.9, 10.6 Hz, 1H), 0.63 (d, J=6.6 Hz, 3H), 0.06 (d, J=6.5 Hz, 3H). ESI-MS m/z calc. 670.29376, found 671.68 (M+1)⁺; Retention time: 2.13 minutes ESI-MS m/z calc. 670.29376, found 671.68 (M+1)⁺; Retention time: 2.13 minutes; LC method A.

### Example 154: Preparation of Compound 406 and Compound 407

### Step 1: (2R)-2-isobutyl-7-(5-isopropoxypyrimidin-2-yl)azepan-3-ol isomer 1 and (2R)-2-isobutyl-7-(5-isopropoxypyrimidin-2-yl)azepan-3-ol as a diastereomeric mixture

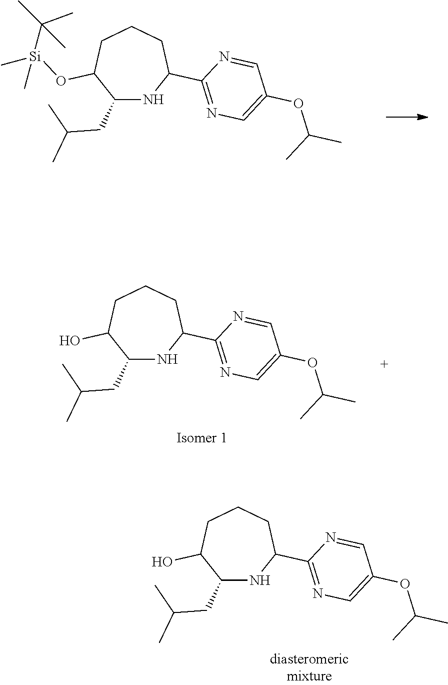

To a solution of tert-butyl-[(2R)-2-isobutyl-7-(5-isopropoxypyrimidin-2-yl)azepan-3-yl]oxy-dimethyl-silane (368 mg, 0.8727 mmol) in MeOH (13.25 mL) was added HCl (10.5 mL of 3 M, 31.500 mmol) and HCl (0.785 mL of 12 M, 9.4200 mmol) stirred 12 h at room temperature then heated in a 65° C. oil bath 18 h. The reaction was concentrated in vacuo at 50° C. to a light orange foam and the residue was diluted with EtOAc (20 mL) and added 2 M NaOH (10 mL) and solid NaCl until the aqueous was saturated. The aqueous was extracted with EtOAc (5×20 mL) and the organics were dried over Na₂SO₄, filtered and concentrated (270 mg crude). The residue was purified by flash chromatography (Combiflash, loaded onto a 12 g SiO₂ cartridge with benzene, and eluted with 0-5% MeOH in DCM over a 30 min gradient) to provide (2R)-2-isobutyl-7-(5-isopropoxypyrimidin-2-yl)azepan-3-ol, isomer 1 (28.3 mg, 11%) as a colorless oil as a single enantiomer ESI-MS m/z calc. 307.226, found 308.3 (M+1)⁺; Retention time: 2.94 minutes, ¹H NMR (500 MHz, Chloroform-d) δ 8.33 (s, 2H), 4.59 (hept, J=6.0 Hz, 1H), 4.24 (t, J=5.7, 1H), 3.67 (t, J=4.0 Hz, 1H), 2.94 (dd, J=8.4, 5.8 Hz, 1H), 2.20-2.02 (m, 2H), 1.92-1.83 (m, 1H), 1.82-1.65 (m, 2H), 1.63-1.38 (m, 4H), 1.39-1.34 (m, 6H), 0.97-0.90 (m, 6H) and a mixture of diastereomers containing isomer 2 (2R)-2-isobutyl-7-(5-isopropoxypyrimidin-2-yl)azepan-3-ol (95 mg, 35%) as a colorless oil. ESI-MS m/z calc. 307.226, found 308.4 (M+1)⁺; Retention time: 3.31 minutes 3.03 minutes, 2.73 minutes. LC method S.

### Step 2: 3-[[4-(2,6-Dimethylphenyl)-6-[(2R)-2-isobutyl-7-(5-isopropoxypyrimidin-2-yl)azepan-3-yl]oxy-pyrimidin-2-yl]sulfamoyl]benzoic acid diastereomeric mixture

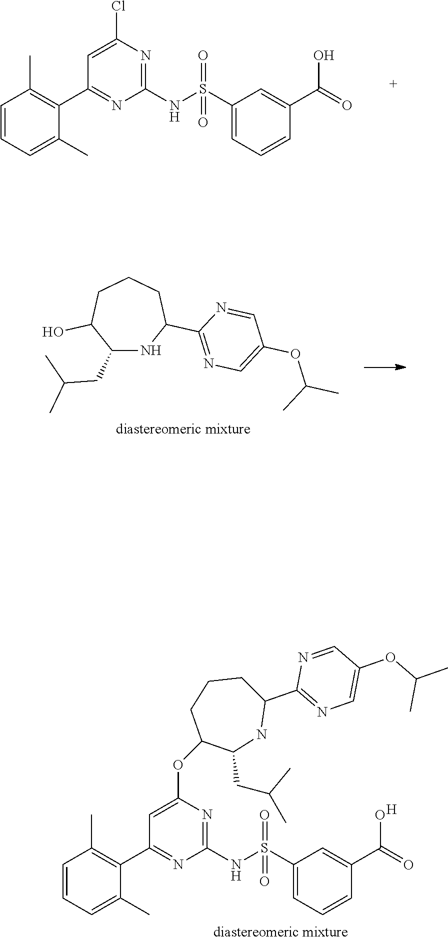

A solution of diastereomers from the previous step of (2R)-2-isobutyl-7-(5-isopropoxypyrimidin-2-yl)azepan-3-ol (95 mg, 0.3090 mmol) in THE (1.9 mL) was added sodium tert-butoxide (150 mg, 1.5608 mmol) at 0° C. The reaction was warmed to room temperature and stirred 10 minutes until a solution formed. Then the reaction was cooled to 0° C. stirred 5 minutes then added 3-[[4-chloro-6-(2,6-dimethylphenyl)pyrimidin-2-yl]sulfamoyl]benzoic acid (155.3 mg, 0.3717 mmol) stirred 15 minutes then warmed to room temperature and stirred for 4 h. The reaction was quenched with aqueous 1M HCl (3 mL), extracted with EtOAc (3×20 mL), washed with brine (20 mL) dried over Na₂SO₄, filtered, and concentrated in vacuo. The residue was triturated in EtOAc (20 mL) for 30 minutes and collected by filtration to provide 3-[[4-(2,6-dimethylphenyl)-6-[(2R)-2-isobutyl-7-(5-isopropoxypyrimidin-2-yl)azepan-3-yl]oxy-pyrimidin-2-yl]sulfamoyl]benzoic acid (65 mg, 30%) as a white solid and as a mixture of diastereomers ESI-MS m/z calc. 688.3043, found 689.7 (M+1)⁺; Retention time: 4.21 minutes and 4.50 minutes. LC method S.

### Step 3: (21R)-12-(2,6-Dimethylphenyl)-21-isobutyl-20-(5-isopropoxypyrimidin-2-yl)-8,8-dioxo-15-oxa-8λ⁶-thia-1,9,11,22-tetrazatetracyclo[14.4.1.13,7.110,14]tricosa-3,5,7(23),10(22),11,13-hexaen-2-one isomer 2

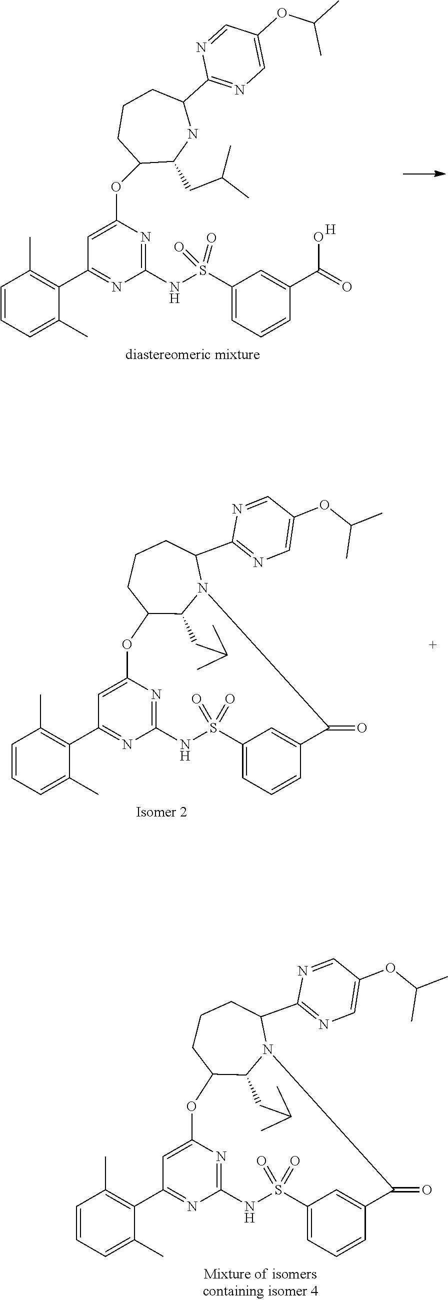

To a solution of 3-[[4-(2,6-dimethylphenyl)-6-[(2R)-2-isobutyl-7-(5-isopropoxypyrimidin-2-yl)azepan-3-yl]oxy-pyrimidin-2-yl]sulfamoyl]benzoic acid (diastereomeric mixture from the previous step, 65 mg, 0.0944 mmol) in DMF (13 mL) was added NMM (57.960 mg, 0.063 mL, 0.5730 mmol) followed by CDMT (33 mg, 0.1880 mmol) at room temperature and the mixture stirred for 60 h. The reaction was quenched with 10% citric acid (10 mL), extracted with EtOAc (3×20 mL), washed with brine (3×30 mL), dried over Na₂SO₄, filtered, and concentrated in vacuo. The residue was purified by flash chromatography (loaded onto a 12 g SiO₂ cartridge with benzene, and eluted with 0-70% EtOAc in Hexanes over a 60 min gradient) to provide (21R)-12-(2,6-dimethylphenyl)-21-isobutyl-20-(5-isopropoxypyrimidin-2-yl)-8,8-dioxo-15-oxa-8λ⁶-thia-1,9,11,22-tetrazatetracyclo[14.4.1.13,7.110,14]tricosa-3,5,7(23),10(22),11,13-hexaen-2-one, isomer 2 (17.1 mg, 27%) as a white solid. ESI-MS m/z calc. 670.2937, found 671.5 (M+1)⁺; Retention time: 3.18 minutes. ¹H NMR (500 MHz, Chloroform-d) δ 8.98 (s, 1H), 8.32 (s, 1H), 8.03 (d, J=7.9 Hz, 2H), 7.76-7.67 (m, 1H), 7.55 (t, J=7.8, 7.8 Hz, 1H), 7.20 (t, J=7.6, 7.6 Hz, 1H), 7.06 (d, J=7.6 Hz, 2H), 6.15 (s, 1H), 5.80 (dt, J=11.9, 4.6 Hz, 1H), 5.31 (t, J=4.9 Hz, 1H), 4.56 (p, J=6.0 Hz, 1H), 4.36-4.28 (m, 1H), 2.51 (dd, J=15.2, 4.5 Hz, 1H), 2.47-2.37 (m, 1H), 2.15-2.07 (m, 1H), 2.01 (s, 6H), 1.98-1.84 (m, 2H), 1.76-1.69 (m, 2H), 1.54 (m, 1H), 1.50-1.43 (m, 1H), 1.38-1.33 (m, 6H), 0.81 (d, J=6.5 Hz, 3H), 0.29 (d, J=6.4 Hz, 3H); LC method W.

A second compound comprised of a diastereomeric mixture containing isomer 4 also eluted to give (21R)-12-(2,6-dimethylphenyl)-21-isobutyl-20-(5-isopropoxy pyrimidin-2-yl)-8,8-dioxo-15-oxa-8λ⁶-thia-1,9,11,22-tetrazatetracyclo[14.4.1.13,7.110,14]tricosa-3,5,7(23),10(22),11,13-hexaen-2-one (9 mg, 14%) as a white solid. ESI-MS m/z calc. 670.2937, found 671.7 (M+1)⁺; Retention time: 6.56 minutes and 6.15 min. LC method S.

### Step 4: 12-(2,6-Dimethylphenyl)-21-isobutyl-20-(5-isopropoxypyrimidin-2-yl)-8,8-dioxo-15-oxa-8λ⁶-thia-1,9,11,22-tetrazatetracyclo[14.4.1.13,7.110,14]tricosa-3,5,7(23),10(22),11,13-hexaen-2-one, isomer 2, SFC peak 1 (Compound 406), and 12-(2,6-dimethylphenyl)-21-isobutyl-20-(5-isopropoxypyrimidin-2-yl)-8,8-dioxo-15-oxa-8λ⁶-thia-1,9,11,22-tetrazatetracyclo[14.4.1.13,7.110,14]tricosa-3,5,7(23),10(22),11,13-hexaen-2-one, isomer 2, SFC peak 2 (Compound 407)

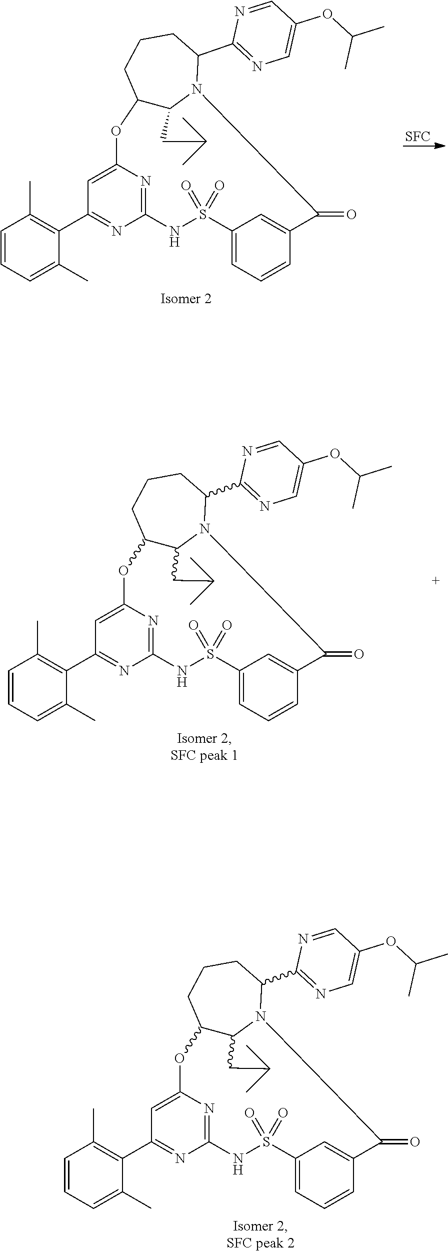 [SMILES: Cc1cccc(C)c1-c1cc2nc(n1)NS(=O)(=O)c1cccc(c1)C(=O)N1C(c3ncc(OC(C)C)cn3)CCCC(O2)[C@H]1CC(C)C ; "SFC" ; Cc1cccc(C)c1-c1cc2nc(n1)NS(=O)(=O)c1cccc(c1)C(=O)N1C(c3ncc(OC(C)C)cn3)CCCC(O2)C1CC(C)C ; Cc1cccc(C)c1-c1cc2nc(n1)NS(=O)(=O)c1cccc(c1)C(=O)N1C(c3ncc(OC(C)C)cn3)CCCC(O2)C1CC(C)C]

(21R)-12-(2,6-Dimethylphenyl)-21-isobutyl-20-(5-isopropoxypyrimidin-2-yl)-8,8-dioxo-15-oxa-8λ⁶-thia-1,9,11,22-tetrazatetracyclo[14.4.1.13,7.110,14]tricosa-3,5,7(23),10(22),11,13-hexaen-2-one (15.6 mg, 0.02326 mmol)(isomer 2, racemic) was subjected to chiral SFC separation using a Chiral Cel OD column (250×10 mm, 5 μM) at 50° C. The mobile phase was MeOH (20 mM NH₃) at a 20 mL/min flow in isocratic mode. Concentration of the sample was 5.6 mg/mL in a mixture of methanol, acetonitrile and DMSO (60:26:14), injection volume 400 μL with a pressure of 181 bar, detection wavelength of 210 nm. For each isomer, the solvents were evaporated to give isomer 2, SFC peak 1, 12-(2,6-dimethylphenyl)-21-isobutyl-20-(5-isopropoxypyrimidin-2-yl)-8,8-dioxo-15-oxa-8λ⁶-thia-1,9,11,22-tetrazatetracyclo[14.4.1.13,7.110,14]tricosa-3,5,7(23),10(22),11,13-hexaen-2-one (6.3 mg, 81%). ¹H NMR (400 MHz, Chloroform-d) δ 8.95 (s, 1H), 8.31 (s, 2H), 8.05 (d, J=7.4 Hz, 1H), 7.67 (broad s, 1H), 7.49 (br s, 1H), 7.17 (t, J=7.6 Hz, 1H), 7.01 (d, J=7.6 Hz, 2H), 6.06 (s, 1H), 5.81 (dt, J=11.6, 4.5 Hz, 1H), 5.29 (t, J=5.0 Hz, 1H), 4.56 (p, J=6.1 Hz, 1H), 4.39-4.24 (m, 1H), 2.54-2.31 (m, 2H), 2.15-2.05 (m, 1H), 1.97 (s, 6H), 1.94-1.82 (m, 2H), 1.77-1.65 (m, 2H), 1.61-1.51 (m, 1H), 1.51-1.38 (m, 1H), 1.39-1.31 (m, 6H), 0.81 (d, J=6.5 Hz, 3H), 0.32 (d, J=6.3 Hz, 3H). ESI-MS m/z calc. 670.29376, found 671.85 (M+1)⁺; Retention time: 2.03 minutes, and isomer 2, SFC peak 2, 12-(2,6-dimethylphenyl)-21-isobutyl-20-(5-isopropoxypyrimidin-2-yl)-8,8-dioxo-15-oxa-8λ⁶-thia-1,9,11,22-tetrazatetracyclo[14.4.1.13,7.110,14]tricosa-3,5,7(23),10(22),11,13-hexaen-2-one (6 mg, 73%). ¹H NMR (400 MHz, Chloroform-d) δ 8.94 (s, 1H), 8.31 (s, 2H), 8.03 (broad s, 1H), 7.64 (br s, 1H), 7.46 (br s, 1H), 7.15 (s, 1H), 7.00 (s, 2H), 6.05 (s, 1H), 5.86-5.71 (m, 1H), 5.34-5.19 (m, 1H), 4.56 (hept, J 6.1 Hz, 1H), 4.39-4.25 (m, 1H), 2.56-2.34 (m, 2H), 2.14-2.02 (m, 1H), 2.14-2.02 (m, 1H), 2.01-1.82 (m, 7H), 1.82-1.65 (m, 2H), 1.58-1.52 (m, 1H), 1.52-1.41 (m, 1H), 1.39-1.32 (m, 6H), 0.81 (d, J=6.4 Hz, 3H), 0.32 (d, J=6.2 Hz, 3H). ESI-MS m/z calc. 670.29376, found 671.63 (M+1)⁺; Retention time: 2.04 minutes; LC method A.

### Example 155: Preparation of Compound 408 and Compound 409

### Step 1: 12-(2,6-Dimethylphenyl)-21-isobutyl-20-(5-isopropoxypyrimidin-2-yl)-8,8-dioxo-15-oxa-8λ⁶-thia-1,9,11,22-tetrazatetracyclo[14.4.1.13,7.110,14]tricosa-3,5,7(23),10(22),11,13-hexaen-2-one, isomer 4, SFC peak 1 (Compound 408), and 12-(2,6-dimethylphenyl)-21-isobutyl-20-(5-isopropoxypyrimidin-2-yl)-8,8-dioxo-15-oxa-8λ⁶-thia-1,9,11,22-tetrazatetracyclo[14.4.1.13,7.110,14]tricosa-3,5,7(23),10(22),11,13-hexaen-2-one, isomer 4, SFC peak 2 (Compound 409)

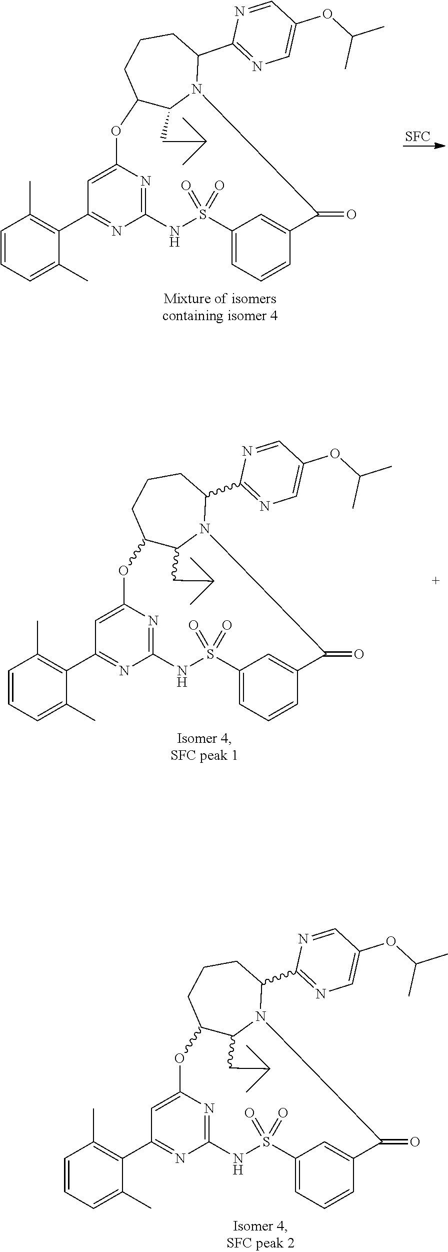 [SMILES: Cc1cccc(C)c1-c1cc2nc(n1)NS(=O)(=O)c1cccc(c1)C(=O)N1C(c3ncc(OC(C)C)cn3)CCCC(O2)[C@H]1CC(C)C ; "SFC" ; Cc1cccc(C)c1-c1cc2nc(n1)NS(=O)(=O)c1cccc(c1)C(=O)N1C(c3ncc(OC(C)C)cn3)CCCC(O2)C1CC(C)C ; Cc1cccc(C)c1-c1cc2nc(n1)NS(=O)(=O)c1cccc(c1)C(=O)N1C(c3ncc(OC(C)C)cn3)CCCC(O2)C1CC(C)C]

The batches containing mixed isomers of (21R)-12-(2,6-dimethylphenyl)-21-isobutyl-20-(5-isopropoxypyrimidin-2-yl)-8,8-dioxo-15-oxa-8λ⁶-thia-1,9,11,22-tetrazatetracyclo[14.4.1.13,7.110,14]tricosa-3,5,7(23),10(22),11,13-hexaen-2-one (25.8 mg, 0.0385 mmol) from several experiments were dissolved in CH₃CN (7 mL) and concentrated in vacuo to provide (21R)-12-(2,6-dimethylphenyl)-21-isobutyl-20-(5-isopropoxypyrimidin-2-yl)-8,8-dioxo-15-oxa-8λ⁶-thia-1,9,11,22-tetrazatetracyclo[14.4.1.13,7.110,14]tricosa-3,5,7(23),10(22),11,13-hexaen-2-one (24.3 mg, 94%) as a white solid as a 70:30 mixture of isomers containing isomer 4. ESI-MS m/z calc. 670.2937, found 671.5 (M+1)⁺; Retention time: 6.59 minutes and 6.18 minutes. LC method S.

(21R)-12-(2,6-Dimethylphenyl)-21-isobutyl-20-(5-isopropoxypyrimidin-2-yl)-8,8-dioxo-15-oxa-8λ⁶-thia-1,9,11,22-tetrazatetracyclo[14.4.1.13,7.110,14]tricosa-3,5,7(23),10(22),11,13-hexaen-2-one (24.3 mg, 0.03622 mmol)(isomer 4, racemic mixed with about 30% of an another unknown stereoisomer) was dissolved in DMSO (2 mL) and purified by reverse phase HPLC (1-99% acetonitrile/5 mM aqueous HCl over 15 min) to give 10.5 mg of white solid. This material was subjected to chiral SFC separation using a Phenomenex Lux-4 column (250×10 mm, 5 μM) at 50° C. The mobile phase was 44% MeOH (20 mM NH₃) at a 20 mL/min flow in isocratic mode. Concentration of the sample was 4.2 mg/mL in mixture of MeOH and DMSO (76:24, v:v). The injection volume was 200 μL with an outlet pressure of 174 bar and the detection wavelength was 210 nm. For each isomer, the solvents were evaporated. The product was dissolved in DMSO (1 mL) and purified by reverse phase HPLC (1-99% acetonitrile/5 mM aqueous HCl over 15 min) to give as colorless resins: isomer 4, SFC peak 1, 12-(2,6-dimethylphenyl)-21-isobutyl-20-(5-isopropoxypyrimidin-2-yl)-8,8-dioxo-15-oxa-8λ⁶-thia-1,9,11,22-tetrazatetracyclo[14.4.1.13,7.110,14]tricosa-3,5,7(23),10(22),11,13-hexaen-2-one (2.1 mg, 17%), ¹H NMR (400 MHz, Chloroform-d) δ 8.65-8.61 (m, 1H), 8.31 (s, 2H), 8.05 (dt, J 7.8, 1.5 Hz, 1H), 7.66 (dt, J=7.6, 1.4 Hz, 1H), 7.58 (t, J=7.7 Hz, 1H), 7.20 (t, J=7.6 Hz, 1H), 7.04 (d, J=7.6 Hz, 2H), 6.08 (s, 1H), 5.68 (dd, J=13.1, 6.4 Hz, 1H), 5.48 (dt, J=11.5, 5.0 Hz, 1H), 4.60 (hept, J=6.1 Hz, 1H), 4.12 (dd, J=8.6, 4.8 Hz, 1H), 2.83 (dt, J=14.7, 7.3 Hz, 1H), 2.27-2.16 (m, 1H), 2.15-2.03 (m, 1H), 2.02-1.88 (m, 7H), 1.88-1.77 (m, 2H), 1.75-1.63 (m, 2H), 1.45-1.34 (m, 6H), 1.32-1.19 (m, 1H), 0.63 (d, J=6.6 Hz, 3H), 0.06 (d, J=6.5 Hz, 3H). ESI-MS m/z calc. 670.29376, found 671.74 (M+1)⁺; Retention time: 2.11 minutes, and isomer 4, SFC peak 2, 12-(2,6-dimethylphenyl)-21-isobutyl-20-(5-isopropoxypyrimidin-2-yl)-8,8-dioxo-15-oxa-8λ⁶-thia-1,9,11,22-tetrazatetracyclo[14.4.1.13,7.110,14]tricosa-3,5,7(23),10(22),11,13-hexaen-2-one (3 mg, 25%), ¹H NMR (400 MHz, Chloroform-d) δ 8.63 (s, 1H), 8.32 (s, 2H), 8.06 (d, J=7.8 Hz, 1H), 7.67 (d, J=7.7 Hz, 1H), 7.59 (t, J=7.7 Hz, 1H), 7.20 (t, J=7.6 Hz, 1H), 7.05 (d, J=7.6 Hz, 2H), 6.11 (s, 1H), 5.68 (dd, J=13.1, 6.3 Hz, 1H), 5.49 (dt, J=11.7, 5.0 Hz, 1H), 4.60 (hept, J=6.0 Hz, 1H), 4.13 (dd, J=8.7, 4.8 Hz, 1H), 2.83 (dt, J=14.7, 7.2 Hz, 1H), 2.29-2.17 (m, 1H), 2.14-2.04 (m, 1H), 2.02-1.91 (m, 7H), 1.86-1.60 (m, 4H), 1.42-1.34 (m, 6H), 1.26 (dd, J=13.8, 10.6 Hz, 1H), 0.64 (d, J=6.6 Hz, 3H), 0.06 (s, 3H). ESI-MS m/z calc. 670.29376, found 671.68 (M+1)⁺; Retention time: 2.12 minutes; LC method A.

### Example 156: Preparation of Compound 410

### Step 1: (NE)-2-Methyl-N-(3-methylbutylidene)propane-2-sulfinamide

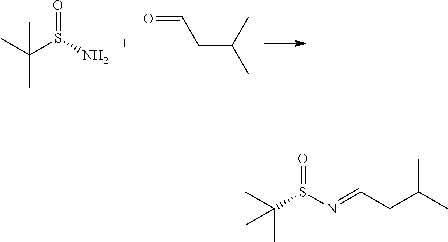

To a stirred solution of (S)-2-methylpropane-2-sulfinamide (10 g, 82.51 mmol) in anhydrous dichloromethane (200 mL) was added 3-methylbutanal (18 mL, 166.3 mmol) followed by addition of Copper (II) Sulfate (40 g, 250.6 mmol) at ambient temperature. The faint blue mixture was stirred at that temperature under nitrogen for 24 h. At the end, the mixture turned slightly blue. The solid was filtered over a short silica gel bed and washed with dichloromethane. The filtrates were concentrated under reduced pressure. Upon further drying under vacuum, desired (NE)-2-methyl-N-(3-methylbutylidene)propane-2-sulfinamide (11.64 g, 74%) was obtained as a light yellow oil. ¹H NMR (400 MHz, DMSO) δ 7.93 (t, J=5.0 Hz, 1H), 2.48-2.34 (m, 2H), 2.10-1.97 (m, 1H), 1.12 (s, 9H), 0.94 (d, J=4.2 Hz, 3H), 0.93 (d, J=4.2 Hz, 3H). ESI-MS m/z calc. 189.11873, found 190.1 (M+1)⁺; Retention time: 1.42 minutes; LC method A.

### Step 2: tert-Butyl-(2-furyloxy)-dimethyl-silane

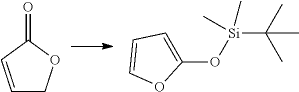

To a stirred solution of 2H-furan-5-one (8 g, 95.15 mmol) in anhydrous dichloromethane (125 mL) was added triethylamine (33 mL, 236.8 mmol) at 0-4° C. (ice-bath) under nitrogen. Then [tert-butyl(dimethyl)silyl]trifluoromethanesulfonate (30 mL, 130.6 mmol) was added slowly over 10 min. After 5 min, the bath was removed, and the reaction was allowed to warm to ambient temperature and continued stirring vigorously the tea-colored solution for 14 h (overnight). The reaction mixture was quenched with water (60 mL). The phases were separated, and organic phase was dried over sodium sulfate, filtered and concentrated under reduced pressure. The residue was passed through a short silica gel plug, to remove baseline materials, furnishing tert-butyl-(2-furyloxy)-dimethyl-silane (17.12 g, 91%) as an orange oil. ¹H NMR (400 MHz, DMSO) δ 6.79 (dd, J=2.2, 1.1 Hz, 1H), 6.06 (dd, J=3.2, 2.2 Hz, 1H), 5.00 (dd, J=3.2, 1.1 Hz, 1H), 0.71 (s, 9H), 0.00 (s, 6H). ESI-MS m/z calc. 198.1076, found 199.1 (M+1)⁺; Retention time: 0.29 minutes; LC method A.

### Step 3: (S)-2-methyl-N-[(1R)-3-methyl-1-[(2S)-5-oxo-2H-furan-2-yl]butyl]propane-2-sulfinamide

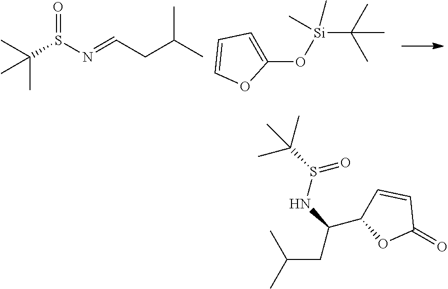

To a stirred solution of tert-butyl-(2-furyloxy)-dimethyl-silane (17.50 g, 88.23 mmol) and (NE)-2-methyl-N-(3-methylbutylidene)propane-2-sulfinamide (11.0 g, 58.10 mmol) in anhydrous dichloromethane (150 mL) was added trimethylsilyl trifluoromethanesulfonate (11 mL, 60.88 mmol) dropwise over 30 min at −78° C. under nitrogen. After stirring for 2 h at that temperature, the reaction was quenched by addition of a saturated aqueous sodium bicarbonate (30 mL) and allowed to slowly warm to about 5° C. The mixture was extracted with dichloromethane (3×50 mL). The combined organics were dried over sodium sulfate, filtered, and concentrated under reduced pressure to obtain an oily residue that was purified by flash chromatography on silica gel (3×330 g silica gel column, 40-60% ethyl acetate in hexanes over 30 min, peak came around 50% ethyl acetate) to furnish a pure fraction and another impure fraction. The front of the peak (fraction A) was discarded. Those two fractions were kept separated. The impure fraction had undesired cis-diastereomer albeit in minor amounts. Impure fraction (B) (mixture of two diastereomers): (S)-2-methyl-N-[(1R)-3-methyl-1-[(2S)-5-oxo-2H-furan-2-yl]butyl]propane-2-sulfinamide (5.61 g, 35%), white solid. ESI-MS m/z calc. 273.13986, found 274.2 (M+1)⁺; Retention time: 1.48 minutes. Fairly pure fraction (C): (S)-2-methyl-N-[(1R)-3-methyl-1-[(2S)-5-oxo-2H-furan-2-yl]butyl]propane-2-sulfinamide (5.07 g, 32%), white solid. ¹H NMR (400 MHz, DMSO) δ 7.70 (dd, J=5.8, 1.5 Hz, 1H), 6.28 (dd, J=5.8, 2.0 Hz, 1H), 5.09 (dt, J=3.9, 1.9 Hz, 1H), 4.90 (d, J=8.2 Hz, 1H), 3.46 (ddt, J=12.3, 8.0, 4.0 Hz, 1H), 1.96-1.84 (m, 1H), 1.59 (ddd, J=14.4, 10.5, 4.3 Hz, 1H), 1.29 (ddd, J=13.7, 9.8, 3.8 Hz, 1H), 1.06 (s, 9H), 0.91 (d, J=6.7 Hz, 3H), 0.87 (d, J=6.5 Hz, 3H). ESI-MS m/z calc. 273.13986, found 274.2 (M+1)⁺; Retention time: 1.48 minutes; LC method A (1-50% gradient).

### Step 4: (S)-2methyl-N-[(1R)-3-methyl-1-[(2S)-5-oxotetrahydrofuran-2-yl]butyl]propane-2-sulfinamide

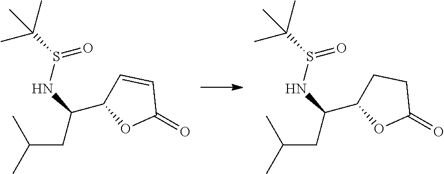 [SMILES: CC(C)C[C@@H](N[S@@](=O)C(C)(C)C)[C@@H]1C=CC(=O)O1 ; CC(C)C[C@@H](N[S@@](=O)C(C)(C)C)[C@@H]1CCC(=O)O1]

Reaction 1 (from impure fraction B): To a stirred solution of (S)-2-methyl-N-[(1R)-3-methyl-1-[(2S)-5-oxo-2H-furan-2-yl]butyl]propane-2-sulfinamide (5.02 g, 18.36 mmol) in ethyl acetate (100 mL) nitrogen gas was sparged for 5 min. Then palladium (3.00 g, 2.819 mmol) was added and continued sparging for another 5 min. The resulting dark suspension was allowed to stir under hydrogen (two balloons) for 40 h. Then the balloons were removed, and the flask was sparged with nitrogen for 10 min. The catalyst was removed over a pad of Celite, and the filter cake was washed with ethyl acetate. The filtrates were concentrated under reduced pressure and purified by flash chromatography on silica gel (isocratic 35% ethyl acetate in hexanes over 20 min) to give an impure lactone (S)-2-methyl-N-[(1R)-3-methyl-1-[(2S)-5-oxotetrahydrofuran-2-yl]butyl]propane-2-sulfinamide (1.55 g, 31%) (white solid, ESI-MS m/z calc. 275.15552, found 276.2 (M+1)⁺; Retention time: 1.53 minutes) as well as pure (S)-2-methyl-N-[(1R)-3-methyl-1-[(2S)-5-oxotetrahydrofuran-2-yl]butyl]propane-2-sulfinamide (3.12 g, 62%) as white solid. ESI-MS m/z calc. 275.15552, found 276.2 (M+1)⁺; Retention time: 1.51 minutes.

Reaction 2 (from pure fraction C): To a stirred solution of (S)-2-methyl-N-[(1R)-3-methyl-1-[(2S)-5-oxo-2H-furan-2-yl]butyl]propane-2-sulfinamide (5.02 g, 18.36 mmol) in ethyl acetate (100 mL), nitrogen gas was sparged for 5 min. Then palladium (3.00 g, 2.819 mmol) was added and continued sparging for another 5 min. The resulting dark suspension was allowed to stir under hydrogen (two balloons) for 40 h. Then the balloons were removed, and the flask was sparged with nitrogen for 10 min. The catalyst was removed over a pad of Celite and the filter cake was washed with ethyl acetate The filtrates were concentrated under reduced pressure and purified by flash chromatography on silica gel (isocratic 35% ethyl acetate in hexanes over 20 min) to give lactone (S)-2-methyl-N-[(1R)-3-methyl-1-[(2S)-5-oxotetrahydrofuran-2-yl]butyl]propane-2-sulfinamide (4.72 g, 93%) as a white solid. ¹H NMR (400 MHz, DMSO) δ 4.98 (d, J=8.0 Hz, 1H), 4.39 (td, J=7.5, 4.7 Hz, 1H), 3.37-3.30 (m, 1H), 2.49-2.45 (m, 1H), 2.38 (ddd, J=17.7, 9.7, 4.8 Hz, 1H), 2.16-2.00 (m, 2H), 1.94-1.80 (m, 1H), 1.47 (ddd, J=14.3, 10.1, 4.4 Hz, 1H), 1.20 (ddd, J=13.8, 9.7, 4.0 Hz, 1H), 1.10 (s, 9H), 0.90 (d, J=6.7 Hz, 3H), 0.87 (d, J=6.5 Hz, 3H). ESI-MS m/z calc. 275.15552, found 276.2 (M+1)⁺; Retention time: 1.53 minutes; LC method A (1-50% gradient).

### Step 5: (5S,6R)-5-Hydroxy-6-isobutyl-piperidin-2-one

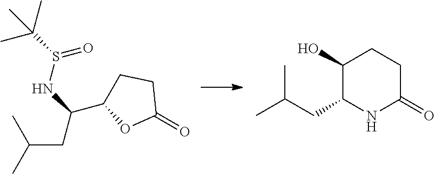 [SMILES: CC(C)C[C@@H](N[S@@](=O)C(C)(C)C)[C@@H]1CCC(=O)O1 ; CC(C)C[C@H]1NC(=O)CC[C@@H]1O]

To a stirred solution of (S)-2-methyl-N-[(1R)-3-methyl-1-[(2S)-5-oxotetrahydrofuran-2-yl]butyl]propane-2-sulfinamide (7.89 g, 28.65 mmol) in anhydrous methanol (300 mL) (mmol HCl to MeOH=1:1) was added hydrogen chloride (4 M in dioxane) (72 mL of 4 M, 288.0 mmol) at ambient temperature under nitrogen. The light yellow solution was allowed to stir at that temperature for 1.5 h. The volatiles were removed under reduced pressure to obtain crude as a brownish gum ((5S)-5-[(1R)-1-amino-3-methyl-butyl]tetrahydrofuran-2-one (hydrochloride salt) (5.990 g, 101%); ESI-MS m/z calc. 171.12593, found 172.1 (M+1)⁺; Retention time: 0.46 minutes). The above gum was taken up in anhydrous methanol (150 mL) and triethylamine (40 mL, 287.0 mmol) was added at 0-4° C. (ice-water bath). The bath was removed, and the reaction was allowed to warm to room temperature. After stirring for 15 h (overnight) at that temperature, the volatiles were removed under reduced pressure. The residue was taken up in dichloromethane (25 mL) and filtered over a pad of celite (to remove triethylamine-HCl salt). The filtrate was concentrated and purified by flash chromatography (2×330 g silica gel column, 0-15% methanol in dichloromethane over 35 min, desired peak (monitored by ELSD) came around 8% methanol) to furnish (5S,6R)-5-hydroxy-6-isobutyl-piperidin-2-one (3.45 g, 70%) as a white solid. ¹H NMR (400 MHz, DMSO) δ 7.26 (s, 1H), 4.92 (d, J=4.0 Hz, 1H), 3.56-3.46 (m, 1H), 3.11 (dtd, J=7.7, 4.8, 2.5 Hz, 1H), 2.22 (ddd, J=17.5, 8.5, 6.6 Hz, 1H), 2.05 (dt, J=17.5, 6.3 Hz, 1H), 1.86-1.71 (m, 2H), 1.64 (dq, J=13.1, 6.5 Hz, 1H), 1.31 (ddd, J=13.6, 8.5, 5.1 Hz, 1H), 1.22 (ddd, J=13.7, 8.2, 5.8 Hz, 1H), 0.87 (d, J=6.6 Hz, 3H), 0.84 (d, J=6.6 Hz, 3H). ESI-MS m/z calc. 171.12593, found 172.2 (M+1)⁺; Retention time: 0.84 minutes, LC method A (1-50% gradient).

### Step 6: (5S,6R)-5-[tert-Butyl(dimethyl)silyl]oxy-6-isobutyl-piperidin-2-one

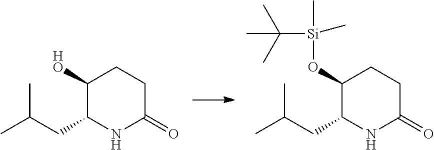 [SMILES: CC(C)C[C@H]1NC(=O)CC[C@@H]1O ; CC(C)C[C@H]1NC(=O)CC[C@@H]1O[Si](C)(C)C(C)(C)C]

To a stirred solution of (5S,6R)-5-hydroxy-6-isobutyl-piperidin-2-one (4 g, 23.36 mmol) in anhydrous N,N-dimethylformamide (50 mL) were added tert-butyl-chloro-dimethyl-silane (4.27 g, 28.33 mmol) and imidazole (4.79 g, 70.36 mmol), in that order at ambient temperature. The resulting clear solution was stirred at that temperature for 15 h (overnight). To the clear reaction water and brine (1:1, 40 mL) was added and extracted with ethyl acetate (3×30 mL). The combined organics were washed with brine (20 mL), dried over anhydrous sodium sulfate, filtered and concentrated under reduced pressure. The clear gum was subjected to flash chromatography (220 g, silica gel column, 30 min run) to furnish desired (5S,6R)-5-[tert-butyl(dimethyl)silyl]oxy-6-isobutyl-piperidin-2-one (5.39 g, 81%) as a clear oil. ¹H NMR (400 MHz, CDCl₃) δ 5.65 (s, 1H), 3.58 (ddd, J=7.8, 5.1, 3.0 Hz, 1H), 3.23-3.12 (m, 1H), 2.47 (ddd, J=17.8, 7.6, 6.3 Hz, 1H), 2.32-2.14 (m, 1H), 1.89-1.79 (m, 1H), 1.77-1.66 (m, 1H), 1.65-1.50 (m, 1H), 1.36 (ddd, J=13.5, 9.5, 4.0 Hz, 1H), 1.20 (ddd, J=14.0, 9.6, 5.0 Hz, 1H), 0.87 (d, J=6.5 Hz, 3H), 0.83 (d, J=6.5 Hz, 3H), 0.81 (s, 9H), 0.00 (s, 6H). ESI-MS m/z calc. 285.2124, found 286.3 (M+1)⁺; Retention time: 1.91 minutes; LC method A.

### Step 7: tert-Butyl (2R,3S)-3-[tert-butyl(dimethyl)silyl]oxy-2-isobutyl-6-oxo-piperidine-1-carboxylate

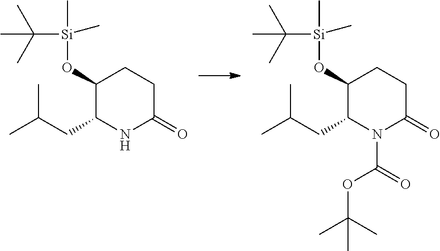 [SMILES: CC(C)C[C@H]1NC(=O)CC[C@@H]1O[Si](C)(C)C(C)(C)C ; CC(C)C[C@@H]1[C@@H](O[Si](C)(C)C(C)(C)C)CCC(=O)N1C(=O)OC(C)(C)C]

To a stirred solution of (5S,6R)-5-[tert-butyl(dimethyl)silyl]oxy-6-isobutyl-piperidin-2-one (2.32 g, 8.126 mmol) in anhydrous tetrahydrofuran (40 mL) was added tert-butoxycarbonyl tert-butyl carbonate (3.50 g, 16.04 mmol), followed by addition of DMAP (1.00 g, 8.185 mmol) at 0-4° C. (ice-water) under nitrogen. The bath was removed and the let the reaction warm to ambient temperature and stirred overnight (16 h). The mixture was concentrated under reduced pressure and the crude was purified flash chromatography (120 g silica gel column, 10-65% ethyl acetate in hexanes over 40 min) to furnish tert-butyl (2R,3S)-3-[tert-butyl(dimethyl)silyl]oxy-2-isobutyl-6-oxo-piperidine-1-carboxylate (2.42 g, 77%) as a colorless gel. ¹H NMR (400 MHz, CDCl₃) δ 4.21-4.14 (m, 1H), 3.91 (q, J=3.1 Hz, 1H), 2.63 (ddd, J=17.7, 10.6, 8.2 Hz, 1H), 2.31 (ddd, J=17.5, 7.7, 2.3 Hz, 1H), 1.97-1.86 (m, 1H), 1.71-1.63 (m, 1H), 1.55 (ddd, J=13.0, 8.4, 6.0 Hz, 1H), 1.42 (s, 9H), 1.35 (ddd, J=14.2, 8.4, 6.0 Hz, 1H), 1.19 (ddd, J=14.1, 8.6, 5.9 Hz, 1H), 0.87 (d, J=6.7 Hz, 3H), 0.84 (d, J=6.6 Hz, 3H), 0.78 (s, 9H), 0.00 (s, 3H), −0.01 (s, 3H). ESI-MS m/z calc. 385.26483, found 286.4 (M+1)⁺; Retention time: 2.28 minutes; LC method A.

### Step 8: tert-Butyl (2R,3S)-3-[tert-butyl(dimethyl)silyl]oxy-2-isobutyl-6-(trifluoromethylsulfonyloxy)-3,4-dihydro-2H-pyridine-1-carboxylate

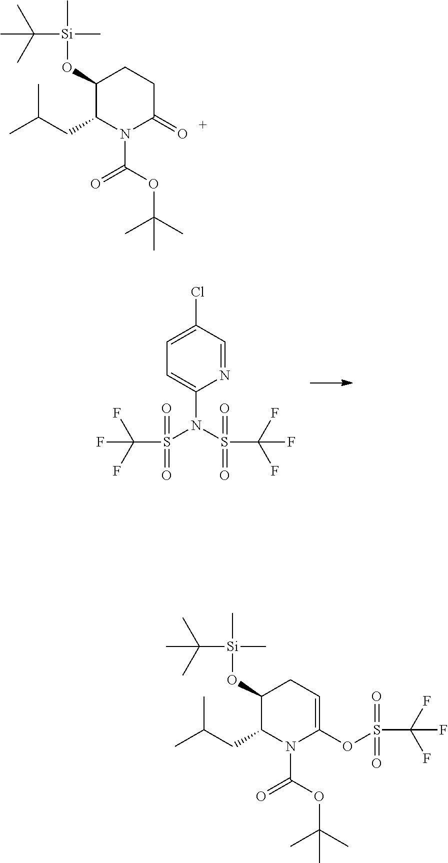 [SMILES: CC(C)C[C@@H]1[C@@H](O[Si](C)(C)C(C)(C)C)CCC(=O)N1C(=O)OC(C)(C)C ; O=S(=O)(N(c1ccc(Cl)cn1)S(=O)(=O)C(F)(F)F)C(F)(F)F ; CC(C)C[C@@H]1[C@@H](O[Si](C)(C)C(C)(C)C)CC=C(OS(=O)(=O)C(F)(F)F)N1C(=O)OC(C)(C)C]

To a solution of tert-butyl (2R,3S)-3-[tert-butyl(dimethyl)silyl]oxy-2-isobutyl-6-oxo-piperidine-1-carboxylate (2.6 g, 6.7425 mmol) in anhydrous THE (50 mL) was added 1.0 M LiHMDS in THE (8.5 mL of 1 M, 8.5000 mmol) dropwise at −78° C. The reaction was stirred at the same temperature for 0.5 hour. A solution of N-(5-chloro-2-pyridyl)-1,1,1-trifluoro-N-(trifluoromethylsulfonyl)methanesulfonamide (3.312 g, 8.4343 mmol) in anhydrous THE (10 mL) was added to the reaction mixture dropwise. The reaction was then stirred at −40° C. for 1 hour. The reaction was quenched with saturated ammonium chloride (50 mL) and then raised to rt. The aqueous solution was extracted with diethyl ether (3×50 mL). The combined organic layers were washed with brine (50 mL), dried over anhydrous sodium sulfate, and concentrated under vacuum. The residue was purified by silica gel chromatography using 0 to 15% diethyl ether in hexane to furnish tert-butyl (2R,3S)-3-[tert-butyl(dimethyl)silyl]oxy-2-isobutyl-6-(trifluoromethylsulfonyloxy)-3,4-dihydro-2H-pyridine-1-carboxylate (3.19 g, 91%) as a clear liquid. ¹H NMR (500 MHz, Chloroform-d) δ 5.12-5.06 (m, 1H), 4.46-4.34 (m, 1H), 3.82-3.77 (m, 1H), 2.31 (dt, J=18.9, 4.0, 4.0 Hz, 1H), 2.14 (dd, J=19.1, 4.2 Hz, 1H), 1.76-1.66 (m, 1H), 1.49 (s, 9H), 1.46-1.40 (m, 1H), 0.98 (d, J=6.3 Hz, 3H), 0.91 (d, J=6.7 Hz, 3H), 0.87 (s, 9H), 0.85-0.79 (m, 1H), 0.16-0.03 (m, 6H). ESI-MS m/z calc. 517.2141, found null (M+)⁺; 462.0 (M−55)⁺; Retention time: 9.26 minutes; LC method S.

### Step 9: tert-Butyl (2R,3S)-3-[tert-butyl(dimethyl)silyl]oxy-2-isobutyl-6-(4,4,5,5-tetramethyl-1,3,2-dioxaborolan-2-yl)-3,4-dihydro-2H-pyridine-1-carboxylate

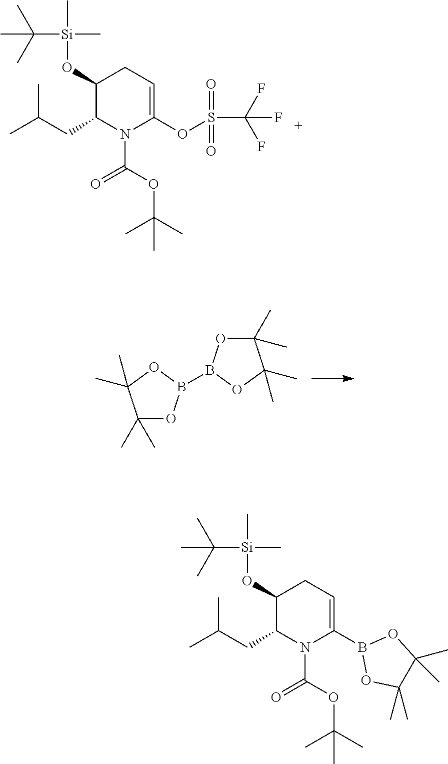

Into a sealed tube was charged with tert-butyl (2R,3S)-3-[tert-butyl(dimethyl)silyl]oxy-2-isobutyl-6-(trifluoromethylsulfonyloxy)-3,4-dihydro-2H-pyridine-1-carboxylate (3.19 g, 6.1622 mmol), 4,4,5,5-tetramethyl-2-(4,4,5,5-tetramethyl-1,3,2-dioxaborolan-2-yl)-1,3,2-dioxaborolane (2.35 g, 9.2542 mmol) and potassium carbonate (2.567 g, 18.574 mmol) in anhydrous dioxane (50 mL). The reaction mixture was purged with argon for 1 hour. Pd(PPh3)₂Cl₂ (433 mg, 0.6169 mmol) and triphenylphosphine (328 mg, 1.2505 mmol) were added to the reaction mixture. The vial was sealed and heated at 90° C. overnight. The reaction was cooled to rt. The solid was removed by filtration through a pad of Celite and washed with ether (50 mL). The combined filtrate was dried over anhydrous sodium sulfate and concentrated under vacuum. The residue was purified by silica gel chromatography using 0 to 15% diethyl ether in hexane to furnish tert-butyl (2R,3S)-3-[tert-butyl(dimethyl)silyl]oxy-2-isobutyl-6-(4,4,5,5-tetramethyl-1,3,2-dioxaborolan-2-yl)-3,4-dihydro-2H-pyridine-1-carboxylate (1.69 g, 55%) as a white solid. ESI-MS m/z calc. 495.35513, found 496.5 (M+1)⁺; Retention time: 5.02 minutes; LC method T.

### Step 10: tert-Butyl (2R,3S)-3-[tert-butyl(dimethyl)silyl]oxy-6-[6-[cyclobutyl(methyl)amino]pyrazin-2-yl]-2-isobutyl-3,4-dihydro-2H-pyridine-1-carboxylate

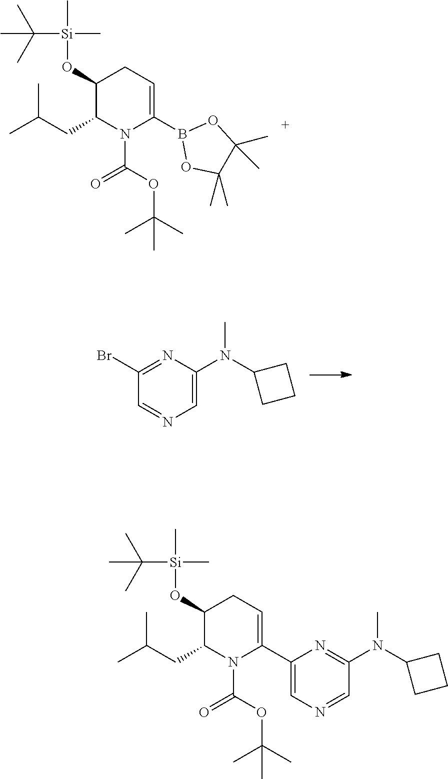 [SMILES: CC(C)C[C@@H]1[C@@H](O[Si](C)(C)C(C)(C)C)CC=C(B2OC(C)(C)C(C)(C)O2)N1C(=O)OC(C)(C)C ; CN(c1cncc(Br)n1)C1CCC1 ; CC(C)C[C@@H]1[C@@H](O[Si](C)(C)C(C)(C)C)CC=C(c2cncc(N(C)C3CCC3)n2)N1C(=O)OC(C)(C)C]

Into a sealed tube was charged with tert-butyl (2R,3S)-3-[tert-butyl(dimethyl)silyl]oxy-2-isobutyl-6-(4,4,5,5-tetramethyl-1,3,2-dioxaborolan-2-yl)-3,4-dihydro-2H-pyridine-1-carboxylate (1.69 g, 3.4102 mmol), 6-bromo-N-cyclobutyl-N-methyl-pyrazin-2-amine (1.25 g, 5.1628 mmol), cesium hydroxide hydrate (1.16 g, 6.9077 mmol). The reaction was purged with argon for 30 minutes. Pd(OAc)₂ (44 mg, 0.1960 mmol) and tris(4-fluorophenyl)phosphine (113 mg, 0.3573 mmol) were added. The tube was sealed. The reaction was heated at 100° C. for 2 hours. After cooled to rt, the reaction was diluted with water (30 mL). Two layers were separated, and the aqueous layer was extracted with ethyl acetate (2×30 mL). The combined organic layers were washed with brine (30 mL), dried over anhydrous sodium sulfate and concentrated under vacuum. The residue was purified by silica gel chromatography using 0 to 20% ethyl acetate in hexane to furnish tert-butyl (2R,3S)-3-[tert-butyl(dimethyl)silyl]oxy-6-[6-[cyclobutyl (methyl)amino]pyrazin-2-yl]-2-isobutyl-3,4-dihydro-2H-pyridine-1-carboxylate (1.55 g, 83%) as a yellow gel. ¹H NMR (500 MHz, Chloroform-d) δ 7.87 (s, 1H), 7.82 (s, 1H), 5.63-5.44 (m, 1H), 4.93-4.70 (m, 1H), 4.48 (d, J=8.5 Hz, 1H), 4.01-3.83 (m, 1H), 3.03 (s, 3H), 2.45-2.33 (m, 1H), 2.29-2.10 (m, 5H), 2.10-1.97 (m, 1H), 1.82-1.64 (m, 2H), 1.50-1.35 (m, 2H), 1.30-0.71 (m, 24H), 0.23-0.05 (m, 6H). ESI-MS m/z calc. 530.36523, found 531.8 (M+1)⁺; Retention time: 4.69 minutes; LC method T.

### Step 11: (2R,3S)-6-[6-[Cyclobutyl(methyl)amino]pyrazin-2-yl]-2-isobutyl-piperidin-3-ol isomer 1

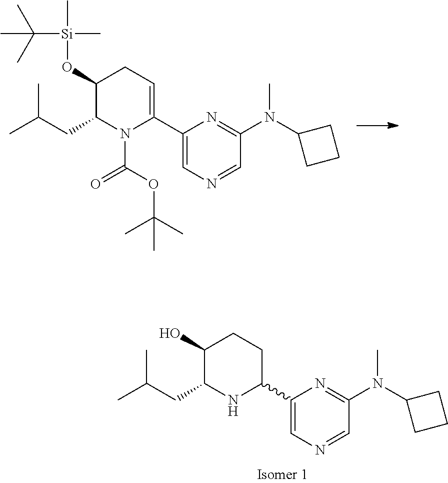 [SMILES: CC(C)C[C@@H]1[C@@H](O[Si](C)(C)C(C)(C)C)CC=C(c2cncc(N(C)C3CCC3)n2)N1C(=O)OC(C)(C)C ; CC(C)C[C@H]1NC(c2cncc(N(C)C3CCC3)n2)CC[C@@H]1O]

Into a reaction flask was charged with tert-butyl (2R,3S)-3-[tert-butyl(dimethyl)silyl]oxy-6-[6-[cyclobutyl(methyl)amino]pyrazin-2-yl]-2-isobutyl-3,4-dihydro-2H-pyridine-1-carboxylate (401 mg, 0.7313 mmol) and 4 N HCl in dioxane (8 mL of 4 M, 32.000 mmol). The reaction mixture was stirred at rt for 2 hours. All the volatiles were removed under vacuum to furnish the crude (2R,3S)-6-[6-[cyclobutyl(methyl)amino]pyrazin-2-yl]-2-isobutyl-1,2,3,4-tetrahydropyridin-3-ol (hydrochloride salt) as an orange gel. ESI-MS m/z calc. 316.2263, found 317.3 (M+1)⁺; Retention time: 2.06 minutes. The crude material was dissolved in anhydrous THE (10 mL). triethylamine (159.72 mg, 0.22 mL, 1.5784 mmol) and sodium triacetoxy borohydride (640 mg, 3.0197 mmol) were added to the reaction mixture at rt. The reaction was stirred at rt for 1.5 hours. The reaction was diluted with 2 N sodium carbonate (20 mL) and ethyl acetate (20 mL). Two layers were separated, and the aqueous layer was extracted with ethyl acetate (4×20 mL). The combined organic layers were dried over anhydrous sodium sulfate and concentrated under vacuum. The residue was purified by silica gel chromatography using 0 to 6% methanol in DCM (buffered with 0.3% ammonium hydroxide) to furnish (2R,3S)-6-[6-[cyclobutyl(methyl)amino]pyrazin-2-yl]-2-isobutyl-piperidin-3-ol, isomer 1 (212 mg, 91%) as a yellow gel. ESI-MS m/z calc. 318.242, found 319.3 (M+1)⁺; Retention time: 1.69 minutes. ESI-MS m/z calc. 316.22632, found 317.3 (M+1)⁺; Retention time: 2.06 minutes; LC method T.

### Step 12: N-[4-Chloro-6-(2,6-dimethylphenyl)pyrimidin-2-yl]-3-[(2R,3S)-6-[6-[cyclobutyl(methyl)amino]pyrazin-2-yl]-3-hydroxy-2-isobutyl-piperidine-1-carbonyl]benzenesulfonamide, isomer 1

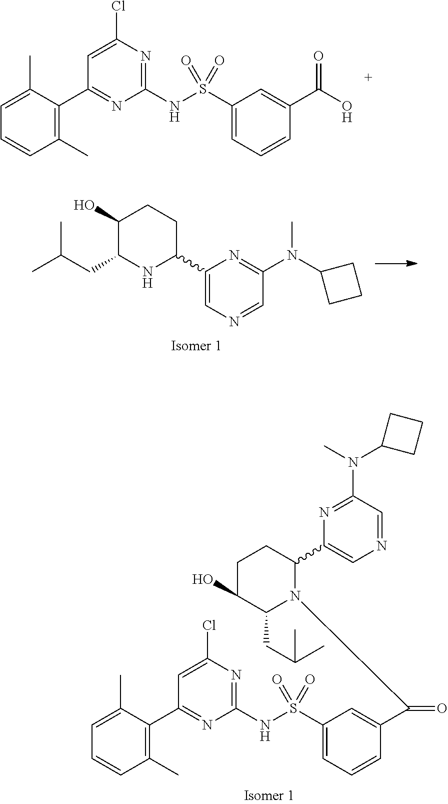 [SMILES: Cc1cccc(C)c1-c1cc(Cl)nc(NS(=O)(=O)c2cccc(C(=O)O)c2)n1 ; CC(C)C[C@H]1NC(c2cncc(N(C)C3CCC3)n2)CC[C@@H]1O ; Cc1cccc(C)c1-c1cc(Cl)nc(NS(=O)(=O)c2cccc(C(=O)N3C(c4cncc(N(C)C5CCC5)n4)CC[C@H](O)[C@H]3CC(C)C)c2)n1]

To a solution of 3-[[4-chloro-6-(2,6-dimethylphenyl)pyrimidin-2-yl]sulfamoyl]benzoic acid (270 mg, 0.6461 mmol) in anhydrous DCM (4 mL) was added thionyl chloride (6.5240 g, 4 mL, 54.837 mmol) The reaction was stirred at 45° C. for 2 days. The volatiles were removed under vacuum to furnish the acid chloride as an off-white foam. The crude acid chloride was dissolved in anhydrous DCM (4 mL). It was added to a solution of (2R,3S)-6-[6-[cyclobutyl(methyl)amino]pyrazin-2-yl]-2-isobutyl-piperidin-3-ol, isomer 1 (162 mg, 0.5087 mmol) and triethylamine (159.72 mg, 0.22 mL, 1.5784 mmol) in anhydrous DCM (4 mL) dropwise at 0° C. The reaction was stirred at rt for 2 hours. The reaction was quenched with 10% citric acid (30 mL) and extracted with ethyl acetate (3×30 mL). The combined organic layers were washed with brine (30 mL), dried over anhydrous sodium sulfate and concentrated under vacuum. The residue was purified by silica gel chromatography using 0 to 50% acetone in hexane to furnish N-[4-chloro-6-(2,6-dimethylphenyl)pyrimidin-2-yl]-3-[(2R,3S)-6-[6-[cyclobutyl(methyl) amino]pyrazin-2-yl]-3-hydroxy-2-isobutyl-piperidine-1-carbonyl]benzenesulfonamide, isomer 1 (133 mg, 33%) as a yellow gel. ¹H NMR (500 MHz, Chloroform-d) δ 8.52-8.35 (m, 1H), 8.21 (d, J=7.8 Hz, 1H), 8.17-8.06 (m, 1H), 7.99-7.85 (m, 1H), 7.80 (d, J=8.4 Hz, 1H), 7.53-7.34 (m, 1H), 7.23-7.14 (m, 1H), 7.10-7.00 (m, 2H), 6.87-6.76 (m, 1H), 6.08 (s, 1H), 5.00-4.75 (m, 1H), 4.70-4.52 (m, 1H), 3.99-3.81 (m, 1H), 3.72 (s, 1H), 3.10-2.84 (m, 3H), 2.49-2.39 (m, 1H), 2.16 (s, 6H), 1.98-1.90 (m, 2H), 1.83-1.62 (m, 4H), 1.22-1.12 (m, 1H), 1.00-0.82 (m, 2H), 0.72 (s, 1H), 0.67-0.56 (m, 1H), 0.56-0.45 (m, 1H), 0.40-0.03 (m, 6H). ESI-MS m/z calc. 717.2864, found 718.4 (M+1)⁺; Retention time: 3.5 minutes; LC method T.

### Step 13: (3S,22R)-6-[6-[Cyclobutyl(methyl)amino]pyrazin-2-yl]-18-(2,6-dimethylphenyl)-22-isobutyl-14,14-dioxo-2-oxa-14λ⁶-thia-7,15,17,20-tetrazatetracyclo[14.3.1.13,7.19,13]docosa-1(19),9,11,13(21),16(20),17-hexaen-8-one, isomer 1 (Compound 410)

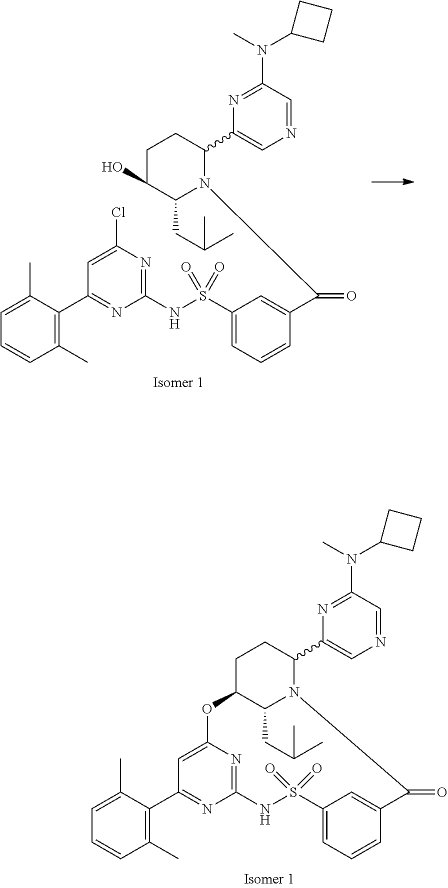 [SMILES: Cc1cccc(C)c1-c1cc(Cl)nc(NS(=O)(=O)c2cccc(C(=O)N3C(c4cncc(N(C)C5CCC5)n4)CC[C@H](O)[C@H]3CC(C)C)c2)n1 ; Cc1cccc(C)c1-c1cc2nc(n1)NS(=O)(=O)c1cccc(c1)C(=O)N1C(c3cncc(N(C)C4CCC4)n3)CC[C@H](O2)[C@H]1CC(C)C]

To a solution of N-[4-chloro-6-(2,6-dimethylphenyl)pyrimidin-2-yl]-3-[(2R,3S)-6-[6-[cyclobutyl(methyl)amino]pyrazin-2-yl]-3-hydroxy-2-isobutyl-piperidine-1-carbonyl]benzenesulfonamide (133 mg, 0.1852 mmol) in anhydrous DMF (8.6 mL) was added NaH (76 mg, 60% w/w, 1.9002 mmol) at 0° C. The reaction was stirred at rt for 24 hours. The reaction was diluted with 10% citric acid (aq.) (10 mL) and extracted with ethyl acetate (5×10 mL). The combined organic layers were washed with brine (10 mL), dried over anhydrous sodium sulfate and concentrated under vacuum. The residue was purified by silica gel chromatography using 0 to 100% ethyl acetate in hexane. The correct fractions were combined and concentrated under vacuum. The residue was purified again with reverse phase prep-HPLC using 0 to 100% acetonitrile-water (buffered with 0.1% TFA) to furnish (3S,22R)-6-[6-[cyclobutyl(methyl)amino]pyrazin-2-yl]-18-(2,6-dimethylphenyl)-22-isobutyl-14,14-dioxo-2-oxa-14λ⁶-thia-7,15,17,20-tetrazatetracyclo[14.3.1.13,7.19,13]docosa-1(19),9,11,13(21),16(20),17-hexaen-8-one (5.9 mg, 4%) as a yellow solid. ¹HNMR (500 MHz, Chloroform-d) δ 8.95 (t, J=1.8 Hz, 1H), 8.13 (s, 1H), 7.93 (s, 1H), 7.76 (d, J=7.9 Hz, 1H), 7.60 (d, J=7.6 Hz, 1H), 7.50 (t, J=7.8 Hz, 1H), 7.22 (t, J=7.6, 7.6 Hz, 1H), 7.06 (d, J=7.8 Hz, 2H), 6.45 (s, 1H), 6.08-5.95 (m, 1H), 4.63 (p, J=8.8, 8.8, 7.9, 7.9 Hz, 1H), 4.15 (s, 1H), 3.56 (dd, J=9.1, 5.9 Hz, 1H), 3.08 (s, 3H), 2.71-2.55 (m, 1H), 2.51-2.37 (m, 2H), 2.35-2.26 (m, 2H), 2.26-2.14 (m, 3H), 1.95 (s, 5H), 1.85-1.71 (m, 2H), 1.02-0.91 (m, 1H), 0.91-0.80 (m, 2H), 0.40 (d, J=6.5 Hz, 3H), −0.08 (d, J=6.5 Hz, 3H). ESI-MS m/z calc. 681.30975, found 682.6 (M+1)⁺; Retention time: 2.77 minutes; LC method W.

### Example 157: Preparation of Compound 411

### Step 1: (2R,3S)-6-[6-[Cyclobutyl(methyl)amino]pyrazin-2-yl]-2-isobutyl-piperidin-3-ol isomer 1 and isomer 2

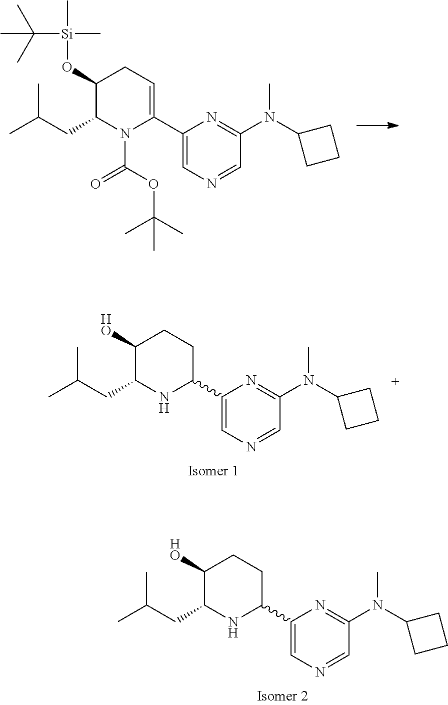 [SMILES: CC(C)C[C@@H]1[C@@H](O[Si](C)(C)C(C)(C)C)CC=C(c2cncc(N(C)C3CCC3)n2)N1C(=O)OC(C)(C)C ; CC(C)C[C@H]1NC(c2cncc(N(C)C3CCC3)n2)CC[C@@H]1O ; CC(C)C[C@H]1NC(c2cncc(N(C)C3CCC3)n2)CC[C@@H]1O]

To a solution of tert-butyl (2R,3S)-3-[tert-butyl(dimethyl)silyl]oxy-6-[6-[cyclobutyl(methyl)amino]pyrazin-2-yl]-2-isobutyl-3,4-dihydro-2H-pyridine-1-carboxylate (651 mg, 1.1872 mmol) in anhydrous DCM (10 mL) was added TFA (8.8800 g, 6 mL, 77.879 mmol) at 0° C. The reaction temperature was slowly raised to rt and stirred for 90 minutes. The volatiles were removed under vacuum to furnish a mixture of 6-[(2R,3S)-3-[tert-butyl(dimethyl)silyl]oxy-2-isobutyl-1,2,3,4-tetrahydropyridin-6-yl]-N-cyclobutyl-N-methyl-pyrazin-2-amine (trifluoroacetate salt) ESI-MS m/z calc. 430.3128, found 431.5 (M+1)⁺; Retention time: 3.48 minutes and (2R,3S)-6-[6-[cyclobutyl(methyl)amino]pyrazin-2-yl]-2-isobutyl-1,2,3,4-tetrahydropyridin-3-ol (trifluoroacetate salt) ESI-MS m/z calc. 316.2263, found 317.3 (M+1)⁺; Retention time: 2.09 minutes, as a red gel. The crude material was dissolved in methanol (10 mL). sodium borohydride (269 mg, 7.1103 mmol) was added to the reaction mixture at 0° C. The reaction was stirred at the same temperature for 10 minutes. The reaction was quenched with concentrated HCl (0.1 mL of 12 M, 1.2000 mmol) at 0° C. The reaction was stirred at rt overnight. Another portion of concentrated HCl (1 mL of 12 M, 12.000 mmol) was added to the reaction mixture. The reaction was stirred at rt for another 24 hours. The reaction was diluted with 2 N sodium carbonate aqueous solution (20 mL). The volatiles were removed under vacuum. The aqueous solution was extracted with ethyl acetate (5×20 mL). The combined organic layers were dried over anhydrous sodium sulfate and concentrated under vacuum. The residue was purified by silica gel chromatography using 0 to 8% methanol in DCM (buffered with 0.3% ammonium hydroxide) to furnish the following two diastereomers, both as yellow gel: Isomer 1: (2R,3S)-6-[6-[cyclobutyl(methyl)amino]pyrazin-2-yl]-2-isobutyl-piperidin-3-ol (146 mg, 37%) ESI-MS m/z calc. 318.242, found 319.1 (M+1)⁺; Retention time: 1.78 minutes; and isomer 2: (2R,3S)-6-[6-[cyclobutyl(methyl)amino]pyrazin-2-yl]-2-isobutyl-piperidin-3-ol (121 mg, 32%), ESI-MS m/z calc. 318.242, found 319.1 (M+1)⁺; Retention time: 1.88 minutes, ¹H NMR (500 MHz, Chloroform-d) δ 7.86 (s, 1H), 7.84 (s, 1H), 4.73-4.45 (m, 1H), 4.00-3.83 (m, 1H), 3.67-3.44 (m, 1H), 3.03 (s, 3H), 2.95-2.85 (m, 1H), 2.78 (s, 2H), 2.32-2.21 (m, 2H), 2.21-2.13 (m, 2H), 2.13-2.02 (m, 1H), 1.96-1.80 (m, 2H), 1.80-1.65 (m, 4H), 1.64-1.51 (m, 1H), 1.43-1.30 (m, 1H), 0.96 (d, J=6.7 Hz, 3H), 0.87 (d, J=6.8 Hz, 3H). LC method T.

### Step 2: (3S,22R)-6-[6-[Cyclobutyl(methyl)amino]pyrazin-2-yl]-18-(2,6-dimethylphenyl)-22-isobutyl-14,14-dioxo-2-oxa-14λ⁶-thia-7,15,17,20-tetrazatetracyclo[14.3.1.13,7.19,13]docosa-1(19),9,11,13(21),16(20),17-hexaen-8-one, isomer 2 (Compound 411)

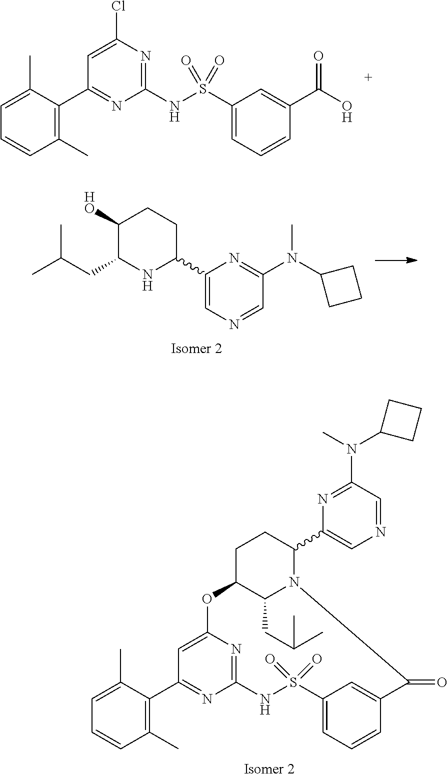 [SMILES: Cc1cccc(C)c1-c1cc(Cl)nc(NS(=O)(=O)c2cccc(C(=O)O)c2)n1 ; CC(C)C[C@H]1NC(c2cncc(N(C)C3CCC3)n2)CC[C@@H]1O ; Cc1cccc(C)c1-c1cc2nc(n1)NS(=O)(=O)c1cccc(c1)C(=O)N1C(c3cncc(N(C)C4CCC4)n3)CC[C@H](O2)[C@H]1CC(C)C]

Into a reaction vial was charged with (2R,3S)-6-[6-[cyclobutyl(methyl)amino]pyrazin-2-yl]-2-isobutyl-piperidin-3-ol, isomer 2 (107 mg, 0.3326 mmol) and 3-[[4-chloro-6-(2,6-dimethylphenyl)pyrimidin-2-yl]sulfamoyl]benzoic acid (157 mg, 0.3757 mmol) in anhydrous THE (2.5 mL). Sodium tert-butoxide (224 mg, 2.3308 mmol) was added to the reaction mixture at rt. The reaction was stirred at rt for 1 hour. The reaction was quenched with 1 N HCl (10 mL) and extracted with ethyl acetate (5×10 mL). The combined organic layers were washed with brine (10 mL), dried over anhydrous sodium sulfate and concentrated under vacuum to afford 3-[[4-[[(2R,3S)-6-[6-[cyclobutyl(methyl)amino]pyrazin-2-yl]-2-isobutyl-3-piperidyl]oxy]-6-(2,6-dimethylphenyl)pyrimidin-2-yl]sulfamoyl]benzoic acid (hydrochloride salt) as yellow solid. ESI-MS m/z calc. 699.3203, found 700.6 (M+1)⁺; Retention time: 2.55 minutes. The crude material was dissolved in anhydrous DMF (20 mL). CDMT (177 mg, 1.0081 mmol) and NMM (202.40 mg, 0.22 mL, 2.0010 mmol) were added to the reaction mixture. The reaction was stirred at rt for 2 days. The reaction was quenched with 10% citric acid (aq.) (30 mL) and extracted with ethyl acetate (3×40 mL). The combined organic layers were washed with brine (3×30 mL), dried over anhydrous sodium sulfate, and concentrated under vacuum. The residue was purified by silica gel chromatography using 30 to 80% ethyl acetate in hexane. The correct fractions were combined and concentrated under vacuum. The residue was further purified by reverse phase HPLC using 0 to 100% acetonitrile in water (buffered with 0.1% TFA) to furnish (3S,22R)-6-[6-[cyclobutyl(methyl)amino]pyrazin-2-yl]-18-(2,6-dimethylphenyl)-22-isobutyl-14,14-dioxo-2-oxa-14λ⁶-thia-7,15,17,20-tetrazatetracyclo[14.3.1.13,7.19,13]docosa-1(19),9,11,13(21),16(20),17-hexaen-8-one (71.6 mg, 310%) as a yellow solid. ¹H NM/R (500 MHz, DMSO-d₆) δ 8.52 (s, 1H), 8.04 (s, 1H), 7.94-7.88 (m, 2H), 7.71 (d, J=6.7 Hz, 1H), 7.66 (t, J=7.6 Hz, 1H), 7.23 (t, J=7.6 Hz, 1H), 7.10 (d, J=8.1 Hz, 2H), 6.33 (s, 1H), 6.00-5.90 (m, 2H), 4.70 (m, 1H), 3.22-3.17 (m, 1H), 3.02 (s, 3H), 2.73-2.66 (m, 1H), 2.36-2.22 (m, 3H), 2.24-2.11 (m, 6H), 1.95 (s, 6H), 1.73-1.60 (m, 2H), 1.08-0.97 (m, 1H), 0.15 (d, J=6.7 Hz, 3H), 0.01 (d, J=6.5 Hz, 3H). ESI-MS m/z calc. 681.30975, found 682.6 (M+1)⁺; Retention time: 2.89 minutes; LC method W.

### VIII. Characterization of Compounds 386-426

The compounds in Table 22 and Table 23 were prepared by procedures disclosed herein, or prepared by procedures analogous to those disclosed herein, and the analytical data were consistent with the reported structure.

TABLE 22
LCMS Data for New Compounds

| | | LCMS | | | |
|---|---|---|---|---|---|
| Compound | | Rt | Calc. | | LCMS |
| No. | Structure | (min) | mass | M + 1 | Method |
| 388 | 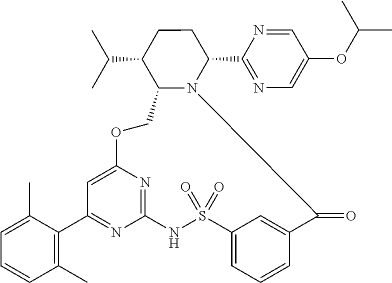 [SMILES: Cc1cccc(C)c1-c1cc2nc(n1)NS(=O)(=O)c1cccc(c1)C(=O)N1[C@@H](c3ncc(OC(C)C)cn3)CC[C@@H](C(C)C)[C@H]1CO2] | 1.97 | 656.278 | 657.5 | A |
| 389 | 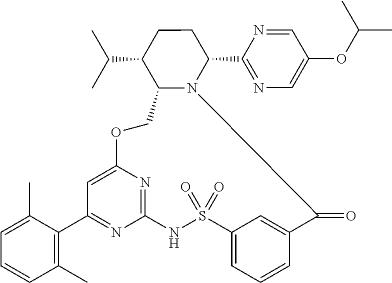 [SMILES: Cc1cccc(C)c1-c1cc2nc(n1)NS(=O)(=O)c1cccc(c1)C(=O)N1[C@@H](c3ncc(OC(C)C)cn3)CC[C@@H](C(C)C)[C@H]1CO2] | 1.97 | 656.278 | 657.5 | A |
| 413 | 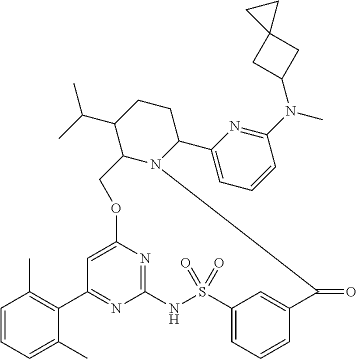 [SMILES: Cc1cccc(C)c1-c1cc2nc(n1)NS(=O)(=O)c1cccc(c1)C(=O)N1C(c3cccc(N(C)C4CC5(CC5)C4)n3)CCC(C(C)C)C1CO2] | 1.85 | 706.33 | 707.8 | A |
| 414 | 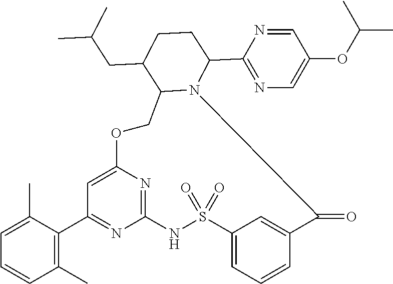 [SMILES: Cc1cccc(C)c1-c1cc2nc(n1)NS(=O)(=O)c1cccc(c1)C(=O)N1C(c3ncc(OC(C)C)cn3)CCC(CC(C)C)C1CO2] | 2.06 | 670.294 | 671.3 | A |
| 390 | 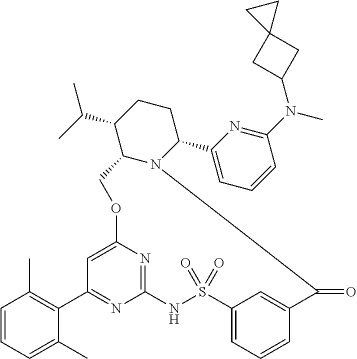 [SMILES: Cc1cccc(C)c1-c1cc2nc(n1)NS(=O)(=O)c1cccc(c1)C(=O)N1[C@@H](c3cccc(N(C)C4CC5(CC5)C4)n3)CC[C@@H](C(C)C)[C@H]1CO2] | 1.84 | 706.33 | 707.4 | A |
| 391 | 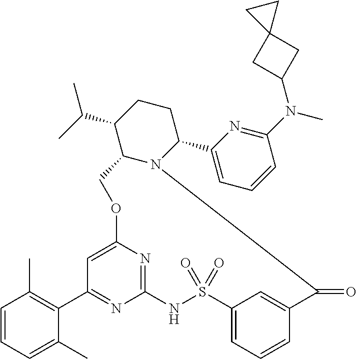 [SMILES: Cc1cccc(C)c1-c1cc2nc(n1)NS(=O)(=O)c1cccc(c1)C(=O)N1[C@@H](c3cccc(N(C)C4CC5(CC5)C4)n3)CC[C@@H](C(C)C)[C@H]1CO2] | 1.84 | 706.33 | 707.4 | A |
| 392 | 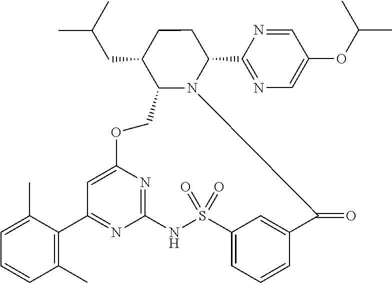 [SMILES: Cc1cccc(C)c1-c1cc2nc(n1)NS(=O)(=O)c1cccc(c1)C(=O)N1[C@@H](c3ncc(OC(C)C)cn3)CC[C@@H](CC(C)C)[C@H]1CO2] | 2.06 | 670.294 | 671.3 | A |
| 393 | 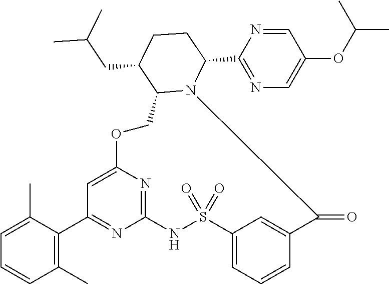 [SMILES: Cc1cccc(C)c1-c1cc2nc(n1)NS(=O)(=O)c1cccc(c1)C(=O)N1[C@@H](c3ncc(OC(C)C)cn3)CC[C@@H](CC(C)C)[C@H]1CO2] | 2.05 | 670.294 | 671.3 | A |
| 415 | 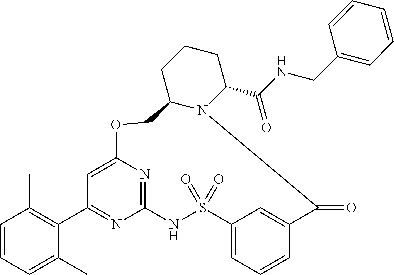 [SMILES: Cc1cccc(C)c1-c1cc2nc(n1)NS(=O)(=O)c1cccc(c1)C(=O)N1[C@H](CCC[C@@H]1C(=O)NCc1ccccc1)CO2] | 2.57 | 611.22 | 612.1 | W |
| 416 | 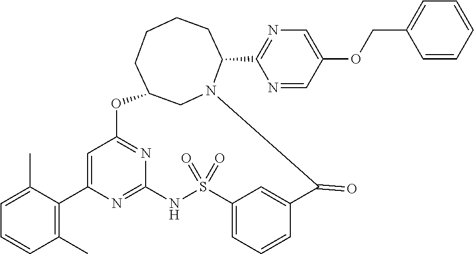 [SMILES: Cc1cccc(C)c1-c1cc2nc(n1)NS(=O)(=O)c1cccc(c1)C(=O)N1C[C@@H](CCCC[C@@H]1c1ncc(OCc3ccccc3)cn1)O2] | 2.01 | 676.247 | 677.78 | A |
| 417 | 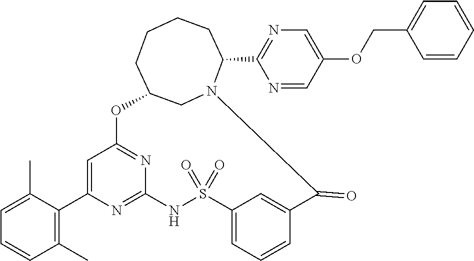 [SMILES: Cc1cccc(C)c1-c1cc2nc(n1)NS(=O)(=O)c1cccc(c1)C(=O)N1C[C@@H](CCCC[C@@H]1c1ncc(OCc3ccccc3)cn1)O2] | 2.01 | 676.247 | 677.83 | A |
| 418 | 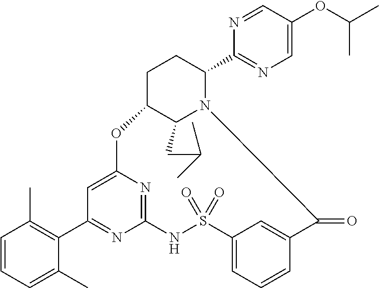 [SMILES: Cc1cccc(C)c1-c1cc2nc(n1)NS(=O)(=O)c1cccc(c1)C(=O)N1[C@@H](c3ncc(OC(C)C)cn3)CC[C@@H](O2)[C@H]1CC(C)C] | 1.22 | 656.278 | 657.008 | A (30-99% gradient) |
| 394 | 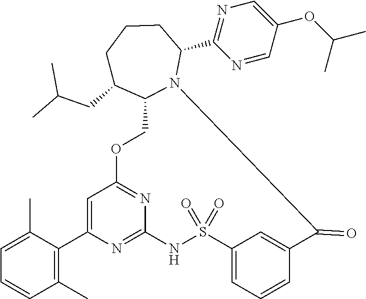 [SMILES: Cc1cccc(C)c1-c1cc2nc(n1)NS(=O)(=O)c1cccc(c1)C(=O)N1[C@@H](c3ncc(OC(C)C)cn3)CCC[C@@H](CC(C)C)[C@H]1CO2] | 2.19 | 684.309 | 685.64 | A |
| 395 | 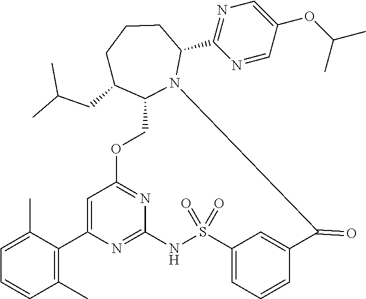 [SMILES: Cc1cccc(C)c1-c1cc2nc(n1)NS(=O)(=O)c1cccc(c1)C(=O)N1[C@@H](c3ncc(OC(C)C)cn3)CCC[C@@H](CC(C)C)[C@H]1CO2] | 2.19 | 684.309 | 685.81 | A |
| 419 | 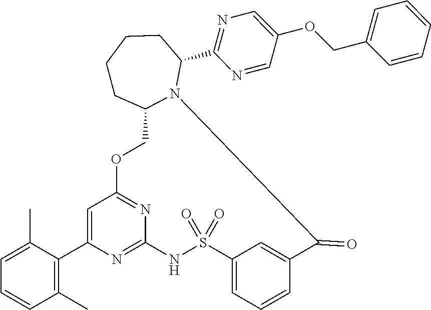 [SMILES: Cc1cccc(C)c1-c1cc2nc(n1)NS(=O)(=O)c1cccc(c1)C(=O)N1[C@@H](CCCC[C@@H]1c1ncc(OCc3ccccc3)cn1)CO2] | 2.05 | 676.247 | 677.38 | A |
| 420 | 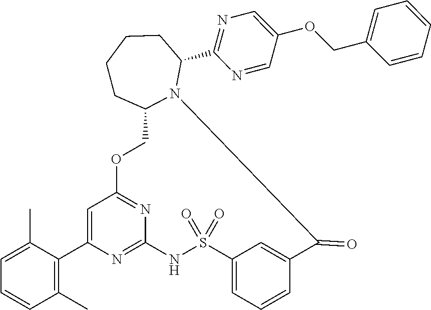 [SMILES: Cc1cccc(C)c1-c1cc2nc(n1)NS(=O)(=O)c1cccc(c1)C(=O)N1[C@@H](CCCC[C@@H]1c1ncc(OCc3ccccc3)cn1)CO2] | 2.07 | 676.247 | 677.53 | A |
| 404 | 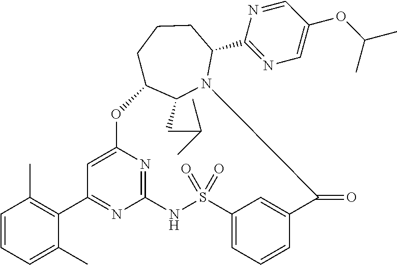 [SMILES: Cc1cccc(C)c1-c1cc2nc(n1)NS(=O)(=O)c1cccc(c1)C(=O)N1[C@@H](c3ncc(OC(C)C)cn3)CCC[C@@H](O2)[C@H]1CC(C)C] | 2.13 | 670.294 | 671.71 | A |
| 398 | 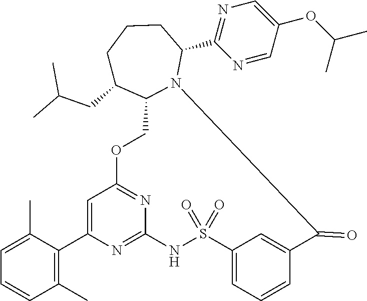 [SMILES: Cc1cccc(C)c1-c1cc2nc(n1)NS(=O)(=O)c1cccc(c1)C(=O)N1[C@@H](c3ncc(OC(C)C)cn3)CCC[C@@H](CC(C)C)[C@H]1CO2] | 2.15 | 684.309 | 685.88 | A |
| 399 | 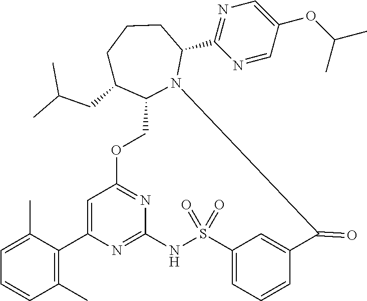 [SMILES: Cc1cccc(C)c1-c1cc2nc(n1)NS(=O)(=O)c1cccc(c1)C(=O)N1[C@@H](c3ncc(OC(C)C)cn3)CCC[C@@H](CC(C)C)[C@H]1CO2] | 2.15 | 684.309 | 685.99 | A |
| 396 | 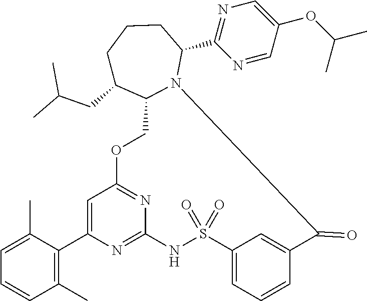 [SMILES: Cc1cccc(C)c1-c1cc2nc(n1)NS(=O)(=O)c1cccc(c1)C(=O)N1[C@@H](c3ncc(OC(C)C)cn3)CCC[C@@H](CC(C)C)[C@H]1CO2] | 2.17 | 684.309 | 685.77 | A |
| 397 | 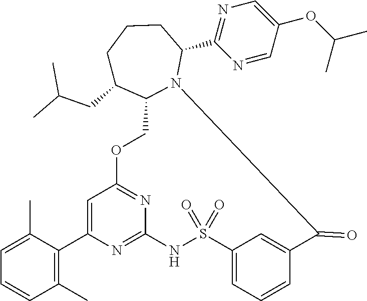 [SMILES: Cc1cccc(C)c1-c1cc2nc(n1)NS(=O)(=O)c1cccc(c1)C(=O)N1[C@@H](c3ncc(OC(C)C)cn3)CCC[C@@H](CC(C)C)[C@H]1CO2] | 2.17 | 684.309 | 685.83 | A |
| 406 | 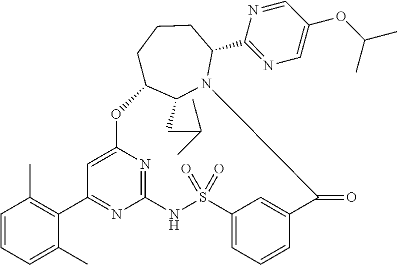 [SMILES: Cc1cccc(C)c1-c1cc2nc(n1)NS(=O)(=O)c1cccc(c1)C(=O)N1[C@@H](c3ncc(OC(C)C)cn3)CCC[C@@H](O2)[C@H]1CC(C)C] | 2.03 | 670.294 | 671.85 | A |
| 407 | 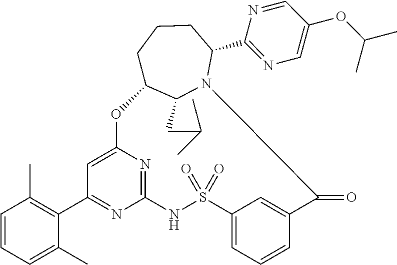 [SMILES: Cc1cccc(C)c1-c1cc2nc(n1)NS(=O)(=O)c1cccc(c1)C(=O)N1[C@@H](c3ncc(OC(C)C)cn3)CCC[C@@H](O2)[C@H]1CC(C)C] | 2.04 | 670.294 | 671.63 | A |
| 421 | 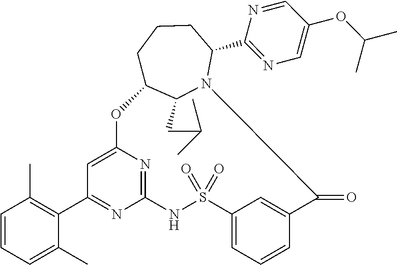 [SMILES: Cc1cccc(C)c1-c1cc2nc(n1)NS(=O)(=O)c1cccc(c1)C(=O)N1[C@@H](c3ncc(OC(C)C)cn3)CCC[C@@H](O2)[C@H]1CC(C)C] | 2.04 | 670.294 | 671.57 | A |
| 422 | 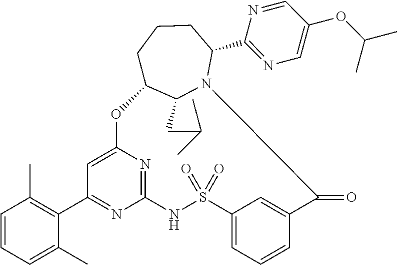 [SMILES: Cc1cccc(C)c1-c1cc2nc(n1)NS(=O)(=O)c1cccc(c1)C(=O)N1[C@@H](c3ncc(OC(C)C)cn3)CCC[C@@H](O2)[C@H]1CC(C)C] | 2.04 | 670.294 | 671.74 | A |
| 423 | 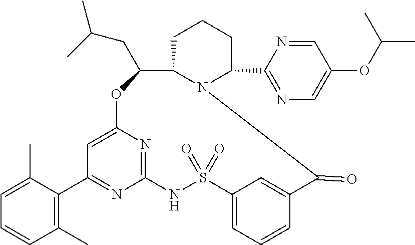 [SMILES: Cc1cccc(C)c1-c1cc2nc(n1)NS(=O)(=O)c1cccc(c1)C(=O)N1[C@@H](c3ncc(OC(C)C)cn3)CCC[C@H]1[C@H](CC(C)C)O2] | 2.1 | 670.294 | 671.68 | A |
| 424 | 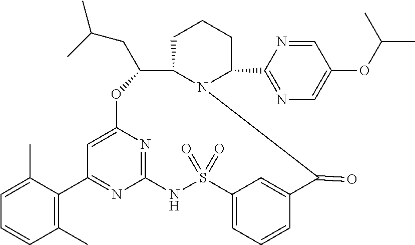 [SMILES: Cc1cccc(C)c1-c1cc2nc(n1)NS(=O)(=O)c1cccc(c1)C(=O)N1[C@@H](c3ncc(OC(C)C)cn3)CCC[C@H]1[C@@H](CC(C)C)O2] | 2.14 | 670.294 | 671.74 | A |
| 408 | 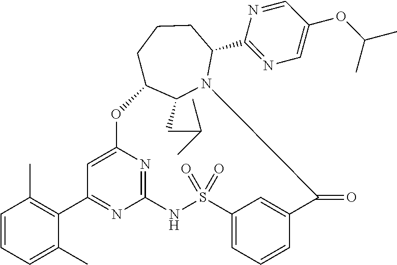 [SMILES: Cc1cccc(C)c1-c1cc2nc(n1)NS(=O)(=O)c1cccc(c1)C(=O)N1[C@@H](c3ncc(OC(C)C)cn3)CCC[C@@H](O2)[C@H]1CC(C)C] | 2.11 | 670.294 | 671.74 | A |
| 409 | 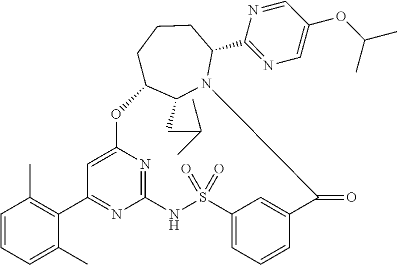 [SMILES: Cc1cccc(C)c1-c1cc2nc(n1)NS(=O)(=O)c1cccc(c1)C(=O)N1[C@@H](c3ncc(OC(C)C)cn3)CCC[C@@H](O2)[C@H]1CC(C)C] | 2.12 | 670.294 | 671.68 | A |
| 425 | 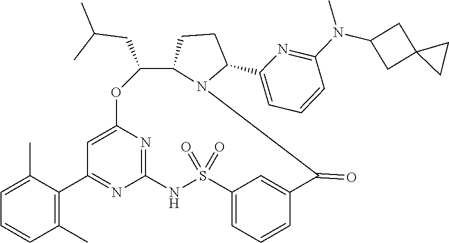 [SMILES: Cc1cccc(C)c1-c1cc2nc(n1)NS(=O)(=O)c1cccc(c1)C(=O)N1[C@@H](c3cccc(N(C)C4CC5(CC5)C4)n3)CC[C@H]1[C@@H](CC(C)C)O2] | 1.66 | 706.33 | 707.84 | A |
| 386 | 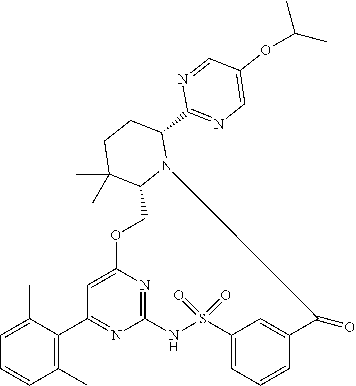 [SMILES: Cc1cccc(C)c1-c1cc2nc(n1)NS(=O)(=O)c1cccc(c1)C(=O)N1[C@@H](c3ncc(OC(C)C)cn3)CCC(C)(C)[C@H]1CO2] | 3.38 | 642.262 | 643.3 | 1B |
| 405 | 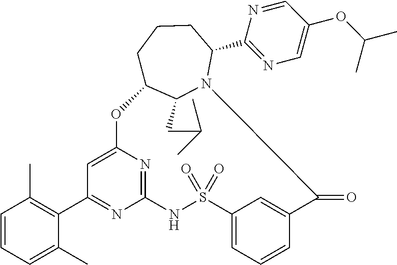 [SMILES: Cc1cccc(C)c1-c1cc2nc(n1)NS(=O)(=O)c1cccc(c1)C(=O)N1[C@@H](c3ncc(OC(C)C)cn3)CCC[C@@H](O2)[C@H]1CC(C)C] | 2.13 | 670.294 | 671.74 | A |
| 426 | 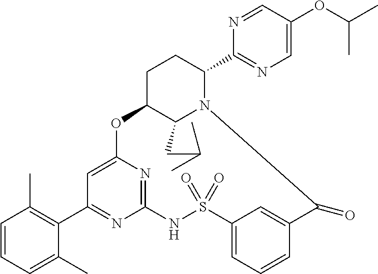 [SMILES: Cc1cccc(C)c1-c1cc2nc(n1)NS(=O)(=O)c1cccc(c1)C(=O)N1[C@@H](c3ncc(OC(C)C)cn3)CC[C@H](O2)[C@H]1CC(C)C] | 1.86 | 656.278 | 657.2 | A |
| 400 | 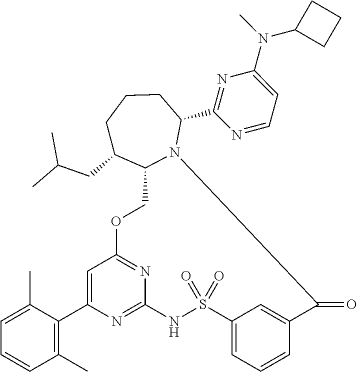 [SMILES: Cc1cccc(C)c1-c1cc2nc(n1)NS(=O)(=O)c1cccc(c1)C(=O)N1[C@@H](c3nccc(N(C)C4CCC4)n3)CCC[C@@H](CC(C)C)[C@H]1CO2] | 2.08 | 709.341 | 710.81 | A |
| 401 | 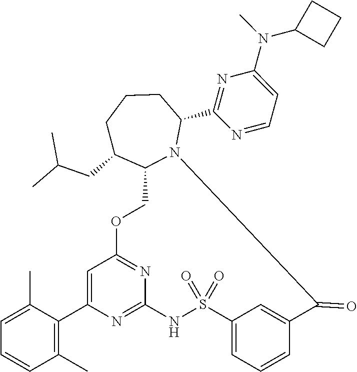 [SMILES: Cc1cccc(C)c1-c1cc2nc(n1)NS(=O)(=O)c1cccc(c1)C(=O)N1[C@@H](c3nccc(N(C)C4CCC4)n3)CCC[C@@H](CC(C)C)[C@H]1CO2] | 2.08 | 709.341 | 710.75 | A |
| 387 | 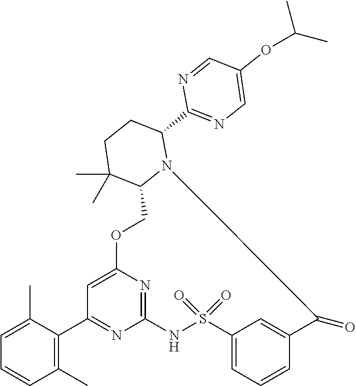 [SMILES: Cc1cccc(C)c1-c1cc2nc(n1)NS(=O)(=O)c1cccc(c1)C(=O)N1[C@@H](c3ncc(OC(C)C)cn3)CCC(C)(C)[C@H]1CO2] | 4.61 | 642.262 | 643.2 | Z |
| 412 | 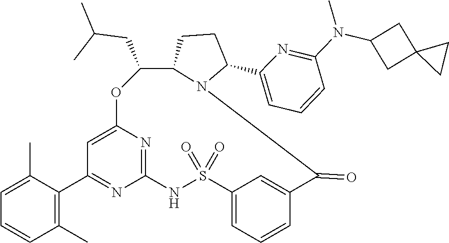 [SMILES: Cc1cccc(C)c1-c1cc2nc(n1)NS(=O)(=O)c1cccc(c1)C(=O)N1[C@@H](c3cccc(N(C)C4CC5(CC5)C4)n3)CC[C@H]1[C@@H](CC(C)C)O2] | 1.7 | 706.33 | 707.89 | A |
| 410 | 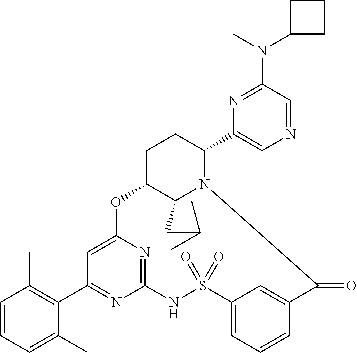 [SMILES: Cc1cccc(C)c1-c1cc2nc(n1)NS(=O)(=O)c1cccc(c1)C(=O)N1[C@@H](c3cncc(N(C)C4CCC4)n3)CC[C@@H](O2)[C@H]1CC(C)C] | 1.96 | 681.31 | 682.8 | A |
| 411 | 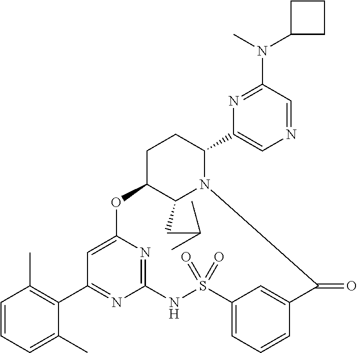 [SMILES: Cc1cccc(C)c1-c1cc2nc(n1)NS(=O)(=O)c1cccc(c1)C(=O)N1[C@@H](c3cncc(N(C)C4CCC4)n3)CC[C@H](O2)[C@H]1CC(C)C] | 1.95 | 681.31 | 682.8 | A |
| 402 | 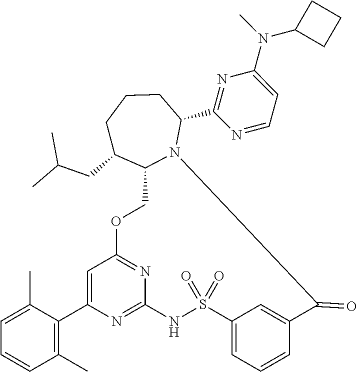 [SMILES: Cc1cccc(C)c1-c1cc2nc(n1)NS(=O)(=O)c1cccc(c1)C(=O)N1[C@@H](c3nccc(N(C)C4CCC4)n3)CCC[C@@H](CC(C)C)[C@H]1CO2] | 1.98 | 709.341 | 710.81 | A |
| 403 | 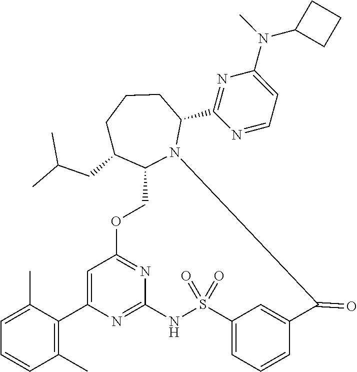 [SMILES: Cc1cccc(C)c1-c1cc2nc(n1)NS(=O)(=O)c1cccc(c1)C(=O)N1[C@@H](c3nccc(N(C)C4CCC4)n3)CCC[C@@H](CC(C)C)[C@H]1CO2] | 1.98 | 709.341 | 710.59 | A |

TABLE 23
NMR Data for New Compounds

| Com- | |
|---|---|
| pound | |
| No. | NMR |
| 392 | ¹H NMR (400 MHZ, Chloroform-d) δ 8.70 (s, 1H), |
| | 8.43 (s, 2H), 7.68 (d, J = 98.5 Hz, 2H), 7.07 (s, 1H), |
| | 6.91 (s, 2H), 6.15 (d, J = 5.9 Hz, 1H), 5.89 (s, 1H), |
| | 5.14 (d, J = 10.6 Hz, 1H), 4.66 (h, J = 6.0 Hz, 1H), |
| | 3.68 (d, J = 11.3 Hz, 1H), 3.40 (d, J = 6.5 Hz, 1H), |
| | 2.54 (d, J = 11.4 Hz, 1H), 2.26-1.72 (m, 11H), 1.50- |
| | 1.32 (m, 7H), 1.03 (dt, J = 13.4, 6.6 Hz, 1H), 0.94- |
| | 0.83 (m, 1H), 0.69 (d, J = 6.3 Hz, 3H), 0.58 (d, J = 6.3 |
| | Hz, 3H). |
| 393 | ¹H NMR (400 MHZ, Chloroform-d) δ 8.70 (s, 1H), |
| | 8.42 (s, 2H), 7.71 (d, J = 106.9 Hz, 2H), 7.07 (s, 1H), |
| | 6.91 (s, 2H), 6.16 (s, 1H), 5.90 (s, 1H), 5.14 (d, J = |
| | 10.7 Hz, 1H), 4.65 (p, J = 6.0 Hz, 1H), 3.68 (d, J = |
| | 11.4 Hz, 1H), 3.50-3.30 (m, 1H), 2.54 (d, J = 11.6 |
| | Hz, 1H), 1.99 (d, J = 126.4 Hz, 11H), 1.40 (t, J = 5.8 |
| | Hz, 7H), 1.03 (t, J = 7.0 Hz, 1H), 0.93-0.82 (m, 1H), |
| | 0.67 (d, J = 6.4 Hz, 3H), 0.57 (d, J = 6.3 Hz, 3H). |
| 415 | ¹H NMR (500 MHZ, DMSO-d₆) δ 8.60 (t, J = 6.1 Hz, |
| | 1H), 7.86 (s, 1H), 7.63 (s, 2H), 7.38-7.30 (m, 5H), |
| | 7.28-7.17 (m, 1H), 7.11 (s, 2H), 6.24 (s, 1H), 5.37 (s, |
| | 1H), 5.12 (s, 1H), 4.40 (dd, J = 14.9, 6.1 Hz, 1H), 4.31 |
| | (dd, J = 15.0, 5.9 Hz, 1H), 4.14 (t, J = 11.6 Hz, 1H), |
| | 3.81-3.72 (m, 1H), 2.30 (d, J = 12.9 Hz, 1H), 2.01 (s, |
| | 6H), 1.74-1.62 (m, 3H), 1.54-1.45 (m, 1H) ,1.41-1.30 |
| | (m, 1H). |
| 416 | ¹H NMR (400 MHZ, DMSO-d₆) δ 12.89 (s, 1H), 8.56 |
| | (s, 2H), 8.41 (s, 1H), 7.85 (d, J = 7.8 Hz, 1H), 7.61 (t, |
| | J = 7.8 Hz, 1H), 7.52-7.46 (m, 2H), 7.46-7.32 (m, |
| | 4H), 7.25 (t, J = 7.7 Hz, 1H), 7.11 (d, J = 7.6 Hz, 2H), |
| | 6.15 (s, 1H), 5.61 (s, 1H), 5.52 (t, J = 7.7 Hz, 1H), |
| | 5.26 (s, 2H), 3.59 (dd, J = 15.1, 11.3 Hz, 1H), 3.39- |
| | 3.28 (m, 1H), 2.31-2.21 (m, 2H), 2.21-1.66 (m, 12H). |
| 417 | ¹H NMR (400 MHZ, DMSO-d₆) δ 13.16-11.74 (broad |
| | m, 1H), 8.56 (s, 2H), 8.41 (s, 1H), 7.85 (d, J = 7.7 Hz, |
| | 1H), 7.62 (t, J = 8.2 Hz, 1H), 7.52-7.46 (m, 2H), 7.45- |
| | 7.33 (m, 4H), 7.24 (t, J = 7.9 Hz, 1H), 7.11 (d, J = |
| | 7.6 Hz, 2H), 6.15 (br s, 1H), 5.61 (s, 1H), 5.57-5.47 |
| | (m, 1H), 5.26 (s, 2H), 3.66-3.50 (m, 1H), 2.31-2.21 |
| | (m, 2H), 2.17-1.63 (m, 12H), one 1H signal masked |
| | under water peak. |
| 418 | ¹H NMR (400 MHZ, Chloroform-d) δ 8.75 (s, 1H), |
| | 8.42 (s, 2H), 8.05 (s, 1H), 7.84 (s, 1H), 7.64 (s, 1H), |
| | 7.21 (t, J = 7.4 Hz, 1H), 7.07 (d, J = 7.4 Hz, 2H), 6.28 |
| | (s, 1H), 6.20 (s, 1H), 6.00 (s, 1H), 4.64 (s, 1H), 3.35 (s, |
| | 1H), 2.92 (s, 1H), 2.31 (s, 3H), 2.03 (s, 6H), 1.39 (t, |
| | J = 4.2 Hz, 6H), 1.26 (s, 3H), 0.88 (s, 1H), 0.22 (d, J = |
| | 37.7 Hz, 6H). |
| 394 | ¹H NMR (400 MHZ, CDCl₃) δ 8.86 (app t, J = 1.8 Hz, |
| | 1H), 8.34 (s, 2H), 8.15 (d, J = 7.9 Hz, 1H), 7.76 (app |
| | d, J = 7.7 Hz, 1H), 7.63 (t, J = 7.8 Hz, 1H), 7.19 (t, |
| | J = 7.6 Hz, 1H), 7.01 (d, J = 7.6 Hz, 2H), 6.17 (s, 1H), |
| | 5.84 (dd, J = 11.8, 5.2 Hz, 1H), 5.73 (dd, J = 13.2, 4.5 |
| | Hz, 1H), 4.65 (t, J = 11.9 Hz, 1H), 4.57 (hept, J = 6.0 |
| | Hz, 1H), 4.30 (dt, J = 12.1, 4.8 Hz, 1H), 2.59-2.50 |
| | (m, 1H), 1.97 (s, 6H), 1.95-1.60 (m, 6H), 1.36 (d, J = |
| | 6.0 Hz, 6H), 1.34-1.19 (m, 2H), 0.93-0.73 (m, 1H), |
| | 0.73-0.66 (m, 6H). |
| 395 | ¹H NMR (400 MHZ, Chloroform-d) δ 8.87 (t, J = 1.9 |
| | Hz, 1H), 8.34 (s, 2H), 8.13 (d, J = 8.0 Hz, 1H), 7.77 |
| | (dt, J = 7.7, 1.4 Hz, 1H), 7.62 (t, J = 7.8 Hz, 1H), 7.19 |
| | (t, J = 7.6 Hz, 1H), 7.02 (d, J = 7.6 Hz, 2H), 6.19 (s, |
| | 1H), 5.83 (dd, J = 11.7, 5.3 Hz, 1H), 5.73 (dd, J = |
| | 13.2, 4.5 Hz, 1H), 4.69-4.61 (m, 1H), 4.60-4.53 (m, |
| | 1H), 4.35-4.22 (m, 1H), 2.60-2.48 (m, 1H), 2.03- |
| | 1.84 (m, 9H), 1.78 (t, J = 9.2 Hz, 2H), 1.68-1.63 (m, |
| | 1H), 1.36 (d, J = 6.1, 1.1 Hz, 6H), 1.25-1.17 (m, 2H), |
| | 0.82-0.73 (m, 1H), 0.73-0.58 (m, 6H). |
| 419 | ¹H NMR (400 MHZ, DMSO-d₆) δ 13.46-11.49 (broad |
| | m, 1H), 8.60 (s, 2H), 8.55 (br s, 1H), 7.85 (br s, 1H), |
| | 7.64 (br s, 2H), 7.51-7.45 (m, 2H), 7.44-7.38 (m, |
| | 2H), 7.38-7.31 (m, 1H), 7.25 (t, J = 7.7 Hz, 1H), 7.12 |
| | (d, J = 7.6 Hz, 2H), 6.25 (s, 1H), 5.70 (dd, J = 11.9, |
| | 5.1 Hz, 1H), 5.48 (d, J = 8.8 Hz, 1H), 5.28 (s, 2H), |
| | 4.37-4.18 (m, 2H), 2.40-2.26 (m, 1H), 2.20-1.93 |
| | (m, 8H), 1.84-1.74 (m, 1H), 1.68-1.55 (m, 3H), 1.54- |
| | 1.45 (m, 1H). ¹H NMR (400 MHZ, CDC13) δ 8.82 |
| | (app t, J = 1.7 Hz, 1H), 8.46 (s, 2H), 7.93 (d, J = 7.9 |
| | Hz, 1H), 7.77 (app d, J = 7.6, 1.4 Hz, 1H), 7.59 (t, J = |
| | 7.8 Hz, 1H), 7.46-7.32 (m, 5H), 7.22 (t, J = 7.6 Hz, |
| | 1H), 7.04 (d, J = 7.6 Hz, 2H), 6.17 (s, 1H), 5.85 (dd, |
| | J = 12.6, 4.8 Hz, 1H), 5.73 (dd, J = 11.0, 4.6 Hz, 1H), |
| | 5.13 (s, 2H), 4.56-4.47 (m, 1H), 4.42 (app q, J = 11.6 |
| | Hz, 1H), 2.61-2.51 (m, 1H), 2.14-2.05 (m, 1H), 2.05- |
| | 1.96 (m, 7H), 1.92-1.84 (m, 1H), 1.76-1.65 (m, 3H), |
| | 1.57-1.49 (m, 1H). |
| 420 | ¹H NMR (400 MHZ, DMSO-d₆) δ 13.44-11.72 (broad |
| | m, 1H), 8.60 (s, 2H), 8.55 (br s, 1H), 7.93-7.79 (br m, |
| | 1H), 7.64 (br s, 2H), 7.53-7.45 (m, 2H), 7.44-7.38 |
| | (m, 2H), 7.38-7.32 (m, 1H), 7.25 (t, J = 7.6 Hz, 1H), |
| | 7.12 (d, J = 7.6 Hz, 2H), 6.26 (br s, 1H), 5.70 (dd, J = |
| | 11.9, 5.1 Hz, 1H), 5.54-5.43 (m, 1H), 5.28 (s, 2H), |
| | 4.36-4.13 (m, 2H), 2.42-2.24 (m, 1H), 2.20-1.91 |
| | (m, 8H), 1.86-1.71 (m, 1H), 1.70-1.43 (m, 4H). |
| 404 | ¹H NMR (400 MHZ, Chloroform-d) δ 9.04 (broad s, |
| | 1H), 8.68-8.56 (m, 1H), 8.31 (s, 2H), 8.06 (dt, J = |
| | 7.9, 1.6 Hz, 1H), 7.66 (dt, J = 7.7, 1.5 Hz, 1H), 7.58 (t, |
| | J = 7.7 Hz, 1H), 7.19 (t, J = 7.6 Hz, 1H), 7.04 (d, J = |
| | 7.6 Hz, 2H), 6.07 (s, 1H), 5.68 (dd, J = 13.1, 6.4 Hz, |
| | 1H), 5.48 (dt, J = 10.9, 5.1 Hz, 1H), 4.60 (hept, J = |
| | 6.6 Hz, 1H), 4.12 (dd, J = 8.7, 4.7 Hz, 1H), 2.83 (dt, |
| | J = 14.8, 7.3 Hz, 1H), 2.30-2.16 (m, 1H), 2.14-2.02 |
| | (m, 1H), 2.00-1.88 (m, 7H), 1.88-1.75 (m, 2H), 1.71- |
| | 1.60 (m, 2H), 1.42-1.33 (m, 6H), 1.25 (dd, J = 13.9, |
| | 10.6 Hz, 1H), 0.63 (d, J = 6.6 Hz, 3H), 0.06 (d, J = 6.5 |
| | Hz, 3H). |

| Com- | |
|---|---|
| pound | |
| number | NMR |
| 396 | ¹H NMR (400 MHZ, Chloroform-d) δ 8.82 (s, 1H), |
| | 8.34 (s, 2H), 8.13 (d, J = 8.0 Hz, 1H), 7.71 (d, J = 7.6 |
| | Hz, 1H), 7.53 (broad s, 1H), 7.14 (t, J = 7.6 Hz, 1H), |
| | 6.96 (d, J = 7.6 Hz, 2H), 6.09 (s, 1H), 5.85 (dd, J = |
| | 11.7, 5.2 Hz, 1H), 5.71 (dd, J = 13.1, 4.4 Hz, 1H), |
| | 4.73-4.50 (m, 2H), 4.29 (dt, J = 12.1, 4.9 Hz, 1H), 2.52 |
| | (dt, J = 14.3, 4.0 Hz, 1H), 2.02-1.84 (m, 9H), 1.81- |
| | 1.71 (m, 2H), 1.70-1.59 (m, 1H), 1.36 (d, J = 6.0 Hz, |
| | 6H), 1.30-1.18 (m, 2H), 0.82-0.75 (m, 1H), 0.74-0.65 |
| | (m, 6H). |
| 397 | ¹H NMR (400 MHZ, Chloroform-d) δ 8.84 (s, 1H), |
| | 8.33 (s, 2H), 8.19 (d, J = 7.9 Hz, 1H), 7.74 (d, J = 8.0 |
| | Hz, 1H), 7.62 (t, J = 7.7 Hz, 1H), 7.17 (t, J = 7.6 Hz, |
| | 1H), 6.98 (d, J = 7.7 Hz, 2H), 6.12 (s, 1H), 5.85 (dd, |
| | J = 11.7, 5.2 Hz, 1H), 5.71 (dd, J = 13.2, 4.5 Hz, 1H), |
| | 4.64 (t, J = 11.9 Hz, 1H), 4.57 (h, J = 6.0 Hz, 1H), |
| | 4.29 (dt, J = 12.1, 4.9 Hz, 1H), 2.53 (dt, J = 15.2, 5.2 |
| | Hz, 1H), 2.07-1.83 (m, 9H), 1.80-1.71 (m, 2H), 1.67 |
| | (dd, J = 11.7, 5.6 Hz, 1H), 1.36 (d, J = 6.0 Hz, 6H), |
| | 1.28 (dt, J = 14.5, 5.2 Hz, 2H), 0.85-0.76 (m, 1H), |
| | 0.75-0.67 (m, 6H). |
| 406 | ¹H NMR (400 MHZ, Chloroform-d) δ 8.95 (s, 1H), |
| | 8.31 (s, 2H), 8.05 (d, J = 7.4 Hz, 1H), 7.67 (broad s, |
| | 1H), 7.49 (br s, 1H), 7.17 (t, J = 7.6 Hz, 1H), 7.01 (d, |
| | J = 7.6 Hz, 2H), 6.06 (s, 1H), 5.81 (dt, J = 11.6, 4.5 Hz, |
| | 1H), 5.29 (t, J = 5.0 Hz, 1H), 4.56 (p, J = 6.1 Hz, 1H), |
| | 4.39-4.24 (m, 1H), 2.54-2.31 (m, 2H), 2.15-2.05 |
| | (m, 1H), 1.97 (s, 6H), 1.94-1.82 (m, 2H), 1.77-1.65 |
| | (m, 2H), 1.61-1.51 (m, 1H), 1.51-1.38 (m, 1H), 1.39- |
| | 1.31 (m, 6H), 0.81 (d, J = 6.5 Hz, 3H), 0.32 (d, J = |
| | 6.3 Hz, 3H). |
| 407 | ¹H NMR (400 MHZ, Chloroform-d) δ 8.94 (s, 1H), |
| | 8.31 (s, 2H), 8.03 (broad s, 1H), 7.64 (br s, 1H), 7.46 |
| | (br s, 1H), 7.15 (s, 1H), 7.00 (s, 2H), 6.05 (s, 1H), 5.86- |
| | 5.71 (m, 1H), 5.34-5.19 (m, 1H), 4.56 (hept, J = 6.1 |
| | Hz, 1H), 4.39-4.25 (m, 1H), 2.56-2.34 (m, 2H), 2.14- |
| | 2.02 (m, 1H), 2.14-2.02 (m, 1H), 2.01-1.82 (m, 7H), |
| | 1.82-1.65 (m, 2H), 1.58-1.52 (m, 1H), 1.52-1.41 (m, |
| | 1H), 1.39-1.32 (m, 6H), 0.81 (d, J = 6.4 Hz, 3H), |
| | 0.32 (d, J = 6.2 Hz, 3H). |
| 421 | ¹H NMR (400 MHZ, Chloroform-d) δ 8.94 (s, 1H), |
| | 8.31 (s, 2H), 8.09-7.97 (broad m, 1H), 7.70-7.61 (br |
| | m, 1H), 7.45 (br s, 1H), 7.16 (t, J = 7.6 Hz, 1H), 7.00 |
| | (d, J = 7.6 Hz, 2H), 6.05 (s, 1H), 5.81 (dt, J = 11.8, |
| | 4.5 Hz, 1H), 5.28 (t, J = 4.9 Hz, 1H), 4.56 (hept, J = |
| | 6.1 Hz, 1H), 4.32 (dt, J = 7.8, 3.3 Hz, 1H), 2.54-2.32 |
| | (m, 2H), 2.14-2.03 (m, 1H), 1.96 (s, 6H), 1.92-1.81 |
| | (m, 2H), 1.77-1.67 (m, 2H), 1.61-1.49 (m, 1H), 1.50- |
| | 1.40 (m, 1H), 1.38-1.31 (m, 6H), 0.81 (d, J = 6.4 |
| | Hz, 3H), 0.32 (d, J = 6.3 Hz, 3H). |
| 422 | ¹H NMR (400 MHZ, Chloroform-d) δ 8.92 (s, 1H), |
| | 8.31 (s, 2H), 7.99 (broad s, 1H), 7.60 (br s, 1H), 7.40 |
| | (br s, 1H), 7.18-7.08 (m, 1H), 6.98 (d, J = 7.6 Hz, |
| | 2H), 6.02 (s, 1H), 5.80 (dt, J = 9.5, 4.3 Hz, 1H), 5.27 |
| | (t, J = 4.8 Hz, 1H), 4.56 (hept, J = 6.0 Hz, 1H), 4.37- |
| | 4.27 (m, 1H), 2.54-2.34 (m, 2H), 2.11-2.01 (m, 1H), |
| | 2.01-1.80 (m, 8H), 1.72 (s, 2H), 1.60-1.51 (m, 1H), |
| | 1.51-1.41 (m, 1H), 1.38-1.31 (m, 6H), 0.81 (d, J = |
| | 6.4 Hz, 3H), 0.32 (d, J = 6.2 Hz, 3H). |
| 423 | ¹H NMR (400 MHZ, CDC13) δ 8.74-8.68 (m, 1H), |
| | 8.43 (s, 2H), 7.74 (d, J = 7.0 Hz, 1H), 7.50 (t, J = 7.6 |
| | Hz, 1H) overlaps with 7.47-7.41 (m, 1H), 7.25 (t, J = |
| | 7.7 Hz, 1H), 7.09 (d, J = 7.6 Hz, 2H), 6.17 (t, J = 7.2 |
| | Hz, 1H), 6.07 (s, 1H), 5.95-5.74 (m, 1H), 4.63 (hept, |
| | J = 6.1 Hz, 1H), 4.00-3.81 (m, 1H), 2.55-2.41 (m, |
| | 1H), 2.35-2.23 (m, 1H), 2.06 (s, 6H), 2.04-1.94 (m, |
| | 1H), 1.95-1.83 (m, 1H), 1.82-1.69 (m, 1H), 1.68- |
| | 1.56 (m, 1H), 1.56-1.45 (m, 1H), 1.41 (d, J = 2.8 Hz, |
| | 3H), 1.40 (d, J = 2.7 Hz, 3H), 1.38-1.29 (m, 1H), 0.78 |
| | (d, J = 6.5 Hz, 3H), 0.65 (d, J = 6.6 Hz, 3H), 0.56-0.41 |
| | (m, 1H). |
| 424 | ¹H NMR (400 MHZ, Chloroform-d) δ 8.76 (s, 1H), |
| | 8.42 (s, 2H), 7.95 (d, J = 7.8 Hz, 1H), 7.80 (d, J = 7.9 |
| | Hz, 1H), 7.62 (t, J = 7.8 Hz, 1H), 7.21 (t, J = 7.6 Hz, |
| | 1H), 7.07 (d, J = 7.6 Hz, 2H), 6.44 (dd, J = 6.6, 2.6 |
| | Hz, 1H), 6.15 (s, 1H), 4.97 (dd, J = 11.3, 4.8 Hz, 1H), |
| | 4.64 (hept, J = 6.0 Hz, 1H), 4.49 (t, J = 11.3 Hz, 1H), |
| | 3.21-3.07 (m, 1H), 2.57-2.40 (m, 2H), 2.05 (s, 6H), |
| | 1.99-1.86 (m, 1H), 1.84-1.73 (m, 1H), 1.70-1.59 |
| | (m, 1H), 1.58-1.49 (m, 1H), 1.45-1.38 (m, 6H), 0.92- |
| | 0.79 (m, 2H), 0.74 (d, J = 6.1 Hz, 3H), 0.58 (d, J = |
| | 6.3 Hz, 3H). |
| 408 | ¹H NMR (400 MHZ, Chloroform-d) δ 8.65-8.61 (m, |
| | 1H), 8.31 (s, 2H), 8.05 (dt, J = 7.8, 1.5 Hz, 1H), 7.66 |
| | (dt, J = 7.6, 1.4 Hz, 1H), 7.58 (t, J = 7.7 Hz, 1H), 7.20 |
| | (t, J = 7.6 Hz, 1H), 7.04 (d, J = 7.6 Hz, 2H), 6.08 (s, |
| | 1H), 5.68 (dd, J = 13.1, 6.4 Hz, 1H), 5.48 (dt, J = |
| | 11.5, 5.0 Hz, 1H), 4.60 (hept, J = 6.1 Hz, 1H), 4.12 |
| | (dd, J = 8.6, 4.8 Hz, 1H), 2.83 (dt, J = 14.7, 7.3 Hz, |
| | 1H), 2.27-2.16 (m, 1H), 2.15-2.03 (m, 1H), 2.02- |
| | 1.88 (m, 7H), 1.88-1.77 (m, 2H), 1.75-1.63 (m, 2H), |
| | 1.45-1.34 (m, 6H), 1.32-1.19 (m, 1H), 0.63 (d, J = |
| | 6.6 Hz, 3H), 0.06 (d, J = 6.5 Hz, 3H). |
| 409 | ¹H NMR (400 MHZ, Chloroform-d) δ 8.63 (s, 1H), |
| | 8.32 (s, 2H), 8.06 (d, J = 7.8 Hz, 1H), 7.67 (d, J = 7.7 |
| | Hz, 1H), 7.59 (t, J = 7.7 Hz, 1H), 7.20 (t, J = 7.6 Hz, |
| | 1H), 7.05 (d, J = 7.6 Hz, 2H), 6.11 (s, 1H), 5.68 (dd, J = |
| | 13.1, 6.3 Hz, 1H), 5.49 (dt, J = 11.7, 5.0 Hz, 1H), |
| | 4.60 (hept, J = 6.0 Hz, 1H), 4.13 (dd, J = 8.7, 4.8 Hz, |
| | 1H), 2.83 (dt, J = 14.7, 7.2 Hz, 1H), 2.29-2.17 (m, |
| | 1H), 2.14-2.04 (m, 1H), 2.02-1.91 (m, 7H), 1.86- |
| | 1.60 (m, 4H), 1.42-1.34 (m, 6H), 1.26 (dd, J = 13.8, |
| | 10.6 Hz, 1H), 0.64 (d, J = 6.6 Hz, 3H), 0.06 (s, 3H). |
| 425 | ¹H NMR (400 MHZ, Chloroform-d) δ 8.97 (s, 1H), |
| | 7.90 (d, J = 7.4 Hz, 1H), 7.77 (t, J = 8.1 Hz, 1H), 7.65- |
| | 7.50 (m, 2H), 7.28 (d, J = 7.3 Hz, 1H), 7.12 (d, J = |
| | 7.6 Hz, 2H), 7.01-6.80 (m, 2H), 6.28 (s, 1H), 6.15 (t, |
| | J = 8.4 Hz, 1H), 5.03-4.83 (m, 2H), 4.36 (t, J = 10.7 |
| | Hz, 1H), 3.64-3.33 (m, 5H), 2.68-2.54 (m, 2H), 2.53- |
| | 2.38 (m, 2H), 2.25-2.03 (m, 8H), 2.02-1.87 (m, |
| | 2H), 1.77-1.56 (m, 1H), 1.04-0.95 (m, 3H), 0.95- |
| | 0.81 (m, 3H), 0.68-0.57 (m, 2H), 0.57-0.43 (m, 2H) |
| 386 | ¹H NMR (400 MHZ, DMSO-d₆) δ 13.19-12.73 (m, |
| | 1H), 8.63 (s, 2H), 8.44 (br. s., 1H), 7.89 (br. s., 1H), |
| | 7.69 (br. s., 2H), 7.29-7.19 (m, 1H), 7.10 (d, J = 7.1 |
| | Hz, 2H), 6.13 (d, J = 4.6 Hz, 2H), 4.85 (quin, J = 6.0 |
| | Hz, 1H), 4.69 (d, J = 9.3 Hz, 1H), 3.48-3.35 (m, 2H), |
| | 2.71-2.63 (m, 1H, overlap with DMSO-D6 satellite), |
| | 2.14-1.92 (m, 8H), 1.33 (m, 7H), 0.84 (s, 3H), 0.79 (s, |
| | 3H). |
| 405 | ¹H NMR (400 MHZ, Chloroform-d) δ 9.04 (broad s, |
| | 1H), 8.66-8.58 (m, 1H), 8.31 (s, 2H), 8.06 (dt, J = |
| | 7.8, 1.5 Hz, 1H), 7.68-7.62 (m, 1H), 7.58 (t, J = 7.7 |
| | Hz, 1H), 7.19 (t, J = 7.6 Hz, 1H), 7.04 (d, J = 7.6 Hz, |
| | 2H), 6.07 (s, 1H), 5.68 (dd, J = 13.1, 6.3 Hz, 1H), 5.53- |
| | 5.39 (m, 1H), 4.60 (hept, J = 5.9 Hz, 1H), 4.12 (dd, |
| | J = 8.6, 4.8 Hz, 1H), 2.90-2.76 (m, 1H), 2.27-2.17 (m, |
| | 1H), 2.14-2.02 (m, 1H), 2.02-1.91 (m, 7H), 1.89- |
| | 1.77 (m, 2H), 1.74-1.63 (m, 2H), 1.44-1.35 (m, 6H), |
| | 1.25 (dd, J = 14.0, 10.5 Hz, 1H), 0.63 (d, J = 6.6 Hz, |
| | 3H), 0.06 (d, J = 6.5 Hz, 3H). |
| 426 | ¹H NMR (400 MHZ, CDC13) δ 9.31 (s, 1H), 8.71 (t, |
| | J = 1.9 Hz, 1H), 8.42 (s, 2H), 7.80 (dt, J = 7.6, 1.4 Hz, |
| | 1H), 7.71 (d, J = 7.9 Hz, 1H), 7.55 (t, J = 7.8 Hz, 1H), |
| | 7.24 (t, J = 7.6 Hz, 1H), 7.07 (d, J = 7.6 Hz, 2H), 6.27 |
| | (d, J = 5.4 Hz, 1H), 6.15 (s, 1H), 6.04-5.92 (m, 1H), |
| | 4.63 (hept, J = 6.1 Hz, 1H), 3.37-3.24 (m, 1H), 2.97- |
| | 2.85 (m, 1H), 2.38-2.22 (m, 3H), 2.01 (s, 6H), 1.39 |
| | (d, J = 6.5 Hz, 3H), 1.37 (d, J = 6.5 Hz, 3H), 1.27- |
| | 1.14 (m, 1H), 0.36-0.28 (m, 1H), 0.26 (d, J = 6.6 Hz, |
| | 3H), 0.24-0.17 (m, 1H), 0.15 (d, J = 6.4 Hz, 3H). |
| 400 | ¹H NMR (400 MHZ, Chloroform-d) δ 8.68-8.63 (m, |
| | 1H), 7.92 (d, J = 7.9 Hz, 1H), 7.87 (s, 1H), 7.84 (s, |
| | 1H), 7.80 (dt, J = 7.7, 1.4 Hz, 1H), 7.59 (t, J = 7.8 Hz, |
| | 1H), 7.22 (t, J = 7.6 Hz, 1H), 7.08 (d, J = 7.6 Hz, 2H), |
| | 6.27 (s, 1H), 5.71 (dd, J = 11.7, 5.4 Hz, 1H), 5.58- |
| | 5.46 (m, 1H), 4.89 (t, J = 11.8 Hz, 1H), 4.49 (p, J = |
| | 8.4 Hz, 1H), 4.40 (dt, J = 11.2, 5.0 Hz, 1H), 3.22 (s, |
| | 3H), 2.39-2.28 (m, 2H), 2.28-2.19 (m, 2H), 2.16- |
| | 2.08 (m, 2H), 2.05 (s, 6H), 1.97-1.88 (m, 2H), 1.87- |
| | 1.79 (m, 1H), 1.80-1.69 (m, 3H), 1.68-1.60 (m, 1H), |
| | 1.27-1.10 (m, 2H), 0.83-0.71 (m, 1H), 0.68-0.60 |
| | (m, 6H). |
| 401 | ¹H NMR (400 MHZ, Chloroform-d) δ 8.66 (s, 1H), |
| | 7.93 (d, J = 7.9 Hz, 1H), 7.87 (s, 1H), 7.83 (s, 1H), |
| | 7.80 (d, J = 7.3 Hz, 1H), 7.59 (t, J = 7.8 Hz, 1H), 7.22 |
| | (t, J = 7.6 Hz, 1H), 7.08 (d, J = 7.6 Hz, 2H), 6.28 (s, |
| | 1H), 5.71 (dd, J = 11.6, 5.4 Hz, 1H), 5.57-5.46 (m, |
| | 1H), 4.89 (t, J = 11.8 Hz, 1H), 4.49 (p, J = 8.4 Hz, |
| | 1H), 4.44-4.32 (m, 1H), 3.22 (s, 3H), 2.38-2.29 (m, |
| | 2H), 2.28-2.19 (m, 2H), 2.17-2.08 (m, 2H), 2.05 (s, |
| | 6H), 1.97-1.88 (m, 2H), 1.88-1.81 (m, 1H), 1.80- |
| | 1.69 (m, 3H), 1.68-1.58 (m, 1H), 1.25-1.12 (m, 2H), |
| | 0.84-0.72 (m, 1H), 0.70-0.57 (m, 6H). |
| 387 | ¹H NMR (400 MHZ, DMSO-d₆) δ 12.95 (br. s, 1H), |
| | 8.63 (s, 2H), 8.44 (s, 1H), 7.89 (br. s, 1H), 7.69 (br. s, |
| | 2H), 7.28-7.20 (m, 1H), 7.10 (d, J = 7.3 Hz, 2H), |
| | 6.13 (d, J = 4.4 Hz, 2H), 4.85 (spt, J = 6.0 Hz, 1H), |
| | 4.69 (d, J = 9.0 Hz, 1H), 3.47-3.35 (m, 2H), 2.71- |
| | 2.64 (m, 1H), 2.17-1.86 (m, 8H), 1.40-1.29 (m, 7H), |
| | 0.84 (s, 3H), 0.79 (s, 3H). |
| 412 | ¹H NMR (400 MHZ, Chloroform-d) δ 14.48 (broad s, |
| | 1H), 10.57 (br s, 1H), 8.66 (s, 1H), 7.70 (d, J = 7.7 Hz, |
| | 2H), 7.42 (t, J = 7.8 Hz, 1H), 7.32 (t, J = 7.6 Hz, 1H), |
| | 7.14 (d, J = 7.6 Hz, 2H), 7.03 (br s, 1H), 6.85 (br s, |
| | 1H), 6.72 (br s, 1H), 6.28-5.67 (m, 3H), 5.05-4.86 |
| | (m, 1H), 3.84 (br s, 1H), 3.42 (br s, 3H), 2.61-2.49 |
| | (m, 2H), 2.42 (br s, 2H), 2.30-1.88 (m, 9H), 1.62 (br |
| | s, 2H), 1.47-1.30 (br m, 1H), 1.16-0.94 (m, 3H), |
| | 0.87 (br s, 3H), 0.64-0.53 (m, 2H), 0.53-0.34 (m, 2H). |
| 410 | ¹H NMR (400 MHZ, Chloroform-d) δ 8.97 (app t, 1H), |
| | 8.14 (s, 1H), 7.96-7.89 (m, 2H), 7.60 (d, 1H), 7.53 (t, |
| | 1H), 7.20 (t, 1H), 7.06 (d, 2H), 6.45 (s, 1H), 6.05- |
| | 6.01 (m, 1H), 4.65 (p, 1H), 4.18-4.14 (m, 1H), 3.58- |
| | 3.50 (m, 1H), 3.08 (s, 3H), 2.68-2.58 (m, 1H), 2.58- |
| | 2.34 (m, 3H), 2.32-2.15 (m, 4H), 1.95 (s, 6H), 1.85- |
| | 1.70 (m, 2H), 1.02-0.93 (m, 1H), 0.91-0.84 (m, 2H), |
| | 0.40 (d, 3H),-0.05 (d, 3H). |
| 411 | ¹H NMR (400 MHZ, Chloroform-d) δ 8.70 (app t, 1H), |
| | 7.99 (s, 1H), 7.95 (s, 1H), 7.76 (app d, 2H), 7.59 (t, |
| | 1H), 7.25 (t, 1H), 7.08 (d, 2H), 6.16 (s, 1H), 6.16- |
| | 6.12 (m, 1H), 6.04-5.93 (m, 1H), 4.64 (p, 1H), 3.34- |
| | 3.23 (m, 1H), 3.12 (s, 3H), 2.93-2.86 (m, 1H), 2.37- |
| | 2.19 (m, 7H), 2.02 (s, 6H), 1.86-1.75 (m, 2H), 1.12- |
| | 1.04 (m, 1H), 0.38-0.27 (m, 1H), 0.24 (d, 3H), 0.21- |
| | 0.13 (m, 1H), 0.12 (d, 3H). |

### IX. Biological Activity Data for Compounds 386-426

A. HBE assay

1. Ussing Chamber Assay of CFTR-Mediated Short-Circuit Currents

Ussing chamber experiments were performed using human bronchial epithelial (HBE) cells derived from CF subjects heterozygous for F508del and a minimal function CFTR mutation (F508del/MF-HBE) and cultured as previously described (Neuberger T, Burton B, Clark H, Van Goor F Methods Mol Biol 2011:741:39-54). After four days the apical media was removed, and the cells were grown at an air liquid interface for >14 days prior to use. This resulted in a monolayer of fully differentiated columnar cells that were ciliated, features that are characteristic of human bronchial airway epithelia.

To isolate the CFTR-mediated short-circuit (I_{SC}) current, F508del/MF-HBE grown on Costar® Snapwell™ cell culture inserts were mounted in an Ussing chamber and the transepithelial I_{SC} was measured under voltage-clamp recording conditions (V_{hold}=0 mV) at 37° C. The basolateral solution contained (in mM) 145 NaCl, 0.83 K₂HPO₄, 3.3 KH₂PO₄, 1.2 MgCl₂, 1.2 CaCl₂, 10 Glucose, 10 HEPES (pH adjusted to 7.4 with NaOH) and the apical solution contained (in mM) 145 NaGluconate, 1.2 MgCl₂, 1.2 CaCl₂, 10 glucose, 10 HEPES (pH adjusted to 7.4 with NaOH) and 30 μM amiloride to block the epithelial sodium channel. Forskolin (20 μM) was added to the apical surface to activate CFTR, followed by apical addition of a CFTR inhibitor cocktail consisting of BPO, GlyH-101, and CFTR inhibitor 172 (each at 20 μM final assay concentration) to specifically isolate CFTR currents. The CFTR-mediated I_{SC} (μA/cm²) for each condition was determined from the peak forskolin response to the steady-state current following inhibition.

2. Identification of Corrector Compounds

The activity of the CFTR corrector compounds on the CFTR-mediated I_{SC} was determined in Ussing chamber studies as described above. The F508del/MF-HBE cell cultures were either incubated with the corrector compounds at a range of concentrations in combination with 1 μM Ivacaftor or were incubated with the corrector compounds at a single fixed concentration of 10 μM in combination with 1 μM Ivacaftor for 18-24 hours at 37° C. and in the presence of 20% human serum. The concentration of corrector compounds with 1 μM Ivacaftor during the 18-24 hours incubations was kept constant throughout the Ussing chamber measurement of the CFTR-mediated I_{SC} to ensure compounds were present throughout the entire experiment. The efficacy and potency of the putative F508del correctors was compared to that of the known Vertex corrector, (14S)-8-[3-(2-{Dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, in combination with 18 μM Tezacaftor and 1 μM Ivacaftor.

B. HBE2 Assay

1. Ussing Chamber Assay of CFTR-Mediated Short-Circuit Currents

Ussing chamber experiments were performed using human bronchial epithelial (HBE) cells derived from CF subjects heterozygous for F508del and a minimal function CFTR mutation (F508del/MF-HBE) and cultured as previously described (Neuberger T, Burton B, Clark H, Van Goor F Methods Mol Biol 2011:741:39-54). After four days the apical media was removed, and the cells were grown at an air liquid interface for >14 days prior to use. This resulted in a monolayer of fully differentiated columnar cells that were ciliated, features that are characteristic of human bronchial airway epithelia.

To isolate the CFTR-mediated short-circuit (I_{SC}) current, F508del/MF-HBE grown on Costar® Snapwell™ cell culture inserts were mounted in an Ussing chamber and the transepithelial I_{SC} was measured under voltage-clamp recording conditions (V_{hold}=0 mV) at 37° C. The basolateral solution contained (in mM) 145 NaCl, 0.83 K₂HPO₄, 3.3 KH₂PO₄, 1.2 MgCl₂, 1.2 CaCl₂, 10 Glucose, 10 HEPES (pH adjusted to 7.4 with NaOH) and the apical solution contained (in mM) 145 NaGluconate, 1.2 MgCl₂, 1.2 CaCl₂, 10 glucose, 10 HEPES (pH adjusted to 7.4 with NaOH) and 30 μM amiloride to block the epithelial sodium channel. Forskolin (20 μM) was added to the apical surface to activate CFTR, followed by apical addition of a CFTR inhibitor cocktail consisting of BPO, GlyH-101, and CFTR inhibitor 172 (each at 20 μM final assay concentration) to specifically isolate CFTR currents. The CFTR-mediated I_{SC} (μA/cm²) for each condition was determined from the peak forskolin response to the steady-state current following inhibition.

2. Identification of Corrector Compounds

The activity of the CFTR corrector compounds on the CFTR-mediated I_{SC} was determined in Ussing chamber studies as described above. The F508del/MF-HBE cell cultures were either incubated with the corrector compounds at a range of concentrations in combination with 44 nM (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol or were incubated with the corrector compounds at a single fixed concentration of 1 and 3 μM in combination with 44 nM (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol for 18-24 hours at 37° C. and in the presence of 2000 human serum. The concentration of corrector compounds with 44 nM (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol during the 18-24 hours incubations was kept constant throughout the Ussing chamber measurement of the CFTR-mediated I_{SC} to ensure compounds were present throughout the entire experiment. The efficacy and potency of the putative F508del correctors was compared to that of the known Vertex corrector, (14S′)-8-[3-(2-{Dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, in combination with 18 M Tezacaftor and 1 μM Ivacaftor.

C. Biological Activity Data Table

Table 24 represents CFTR modulating activity for representative compounds of the invention generated using one or more of the assays disclosed herein (EC₅₀: +++ is <1 μM; ++ is 1-<3 μM; + is 3-<30 μM; and ND is “not detected in this assay.” % Activity: +++ is >60%; ++ is 30-60%; + is <30+).

TABLE 24
Biological Activity Data for Compounds 386-426

| | | | HBE | HBE | HBE2 |
|---|---|---|---|---|---|
| | | HBE | Max | Activity | Activity |
| Compound | | IC₅₀ | activity | at 10 | at 3 μM |
| No. | Structure | (μM) | (%) | μM (%) | (%) |
| 388 | 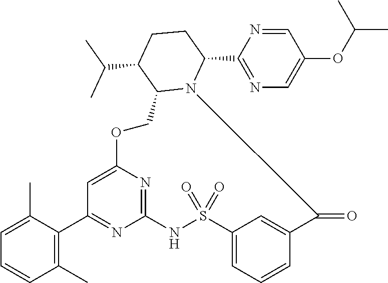 [SMILES: Cc1cccc(C)c1-c1cc2nc(n1)NS(=O)(=O)c1cccc(c1)C(=O)N1[C@@H](c3ncc(OC(C)C)cn3)CC[C@@H](C(C)C)[C@H]1CO2] | +++ | +++ | | |
| 389 | 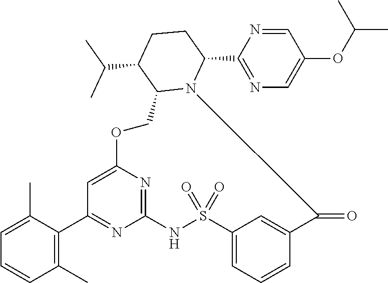 [SMILES: Cc1cccc(C)c1-c1cc2nc(n1)NS(=O)(=O)c1cccc(c1)C(=O)N1[C@@H](c3ncc(OC(C)C)cn3)CC[C@@H](C(C)C)[C@H]1CO2] | ++ | + | | |
| 413 | 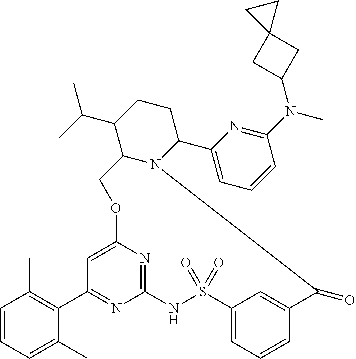 [SMILES: Cc1cccc(C)c1-c1cc2nc(n1)NS(=O)(=O)c1cccc(c1)C(=O)N1C(c3cccc(N(C)C4CC5(CC5)C4)n3)CCC(C(C)C)C1CO2] | | | | |
| 414 | 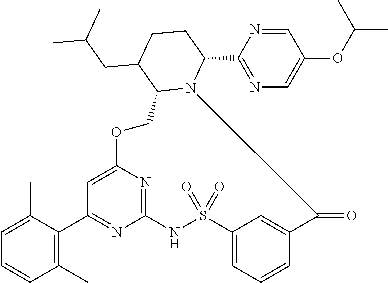 [SMILES: Cc1cccc(C)c1-c1cc2nc(n1)NS(=O)(=O)c1cccc(c1)C(=O)N1[C@@H](c3ncc(OC(C)C)cn3)CCC(CC(C)C)[C@H]1CO2] | | | +++ | |
| 390 | 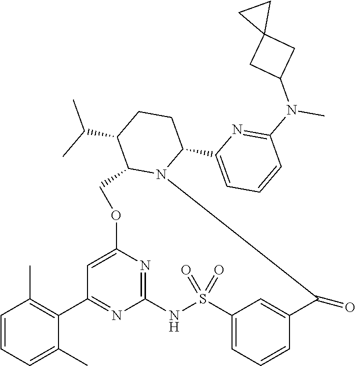 [SMILES: Cc1cccc(C)c1-c1cc2nc(n1)NS(=O)(=O)c1cccc(c1)C(=O)N1[C@@H](c3cccc(N(C)C4CC5(CC5)C4)n3)CC[C@@H](C(C)C)[C@H]1CO2] | | | +++ | |
| 391 | 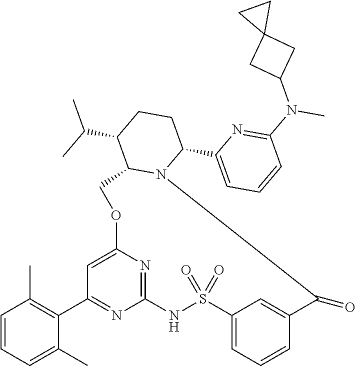 [SMILES: Cc1cccc(C)c1-c1cc2nc(n1)NS(=O)(=O)c1cccc(c1)C(=O)N1[C@@H](c3cccc(N(C)C4CC5(CC5)C4)n3)CC[C@@H](C(C)C)[C@H]1CO2] | | | ++ | |
| 392 | 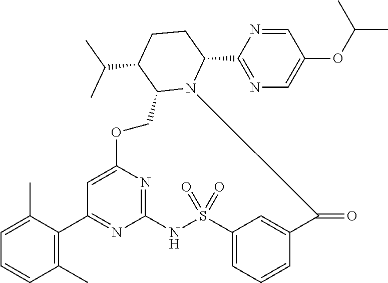 [SMILES: Cc1cccc(C)c1-c1cc2nc(n1)NS(=O)(=O)c1cccc(c1)C(=O)N1[C@@H](c3ncc(OC(C)C)cn3)CC[C@@H](C(C)C)[C@H]1CO2] | | | ++ | |
| 393 | 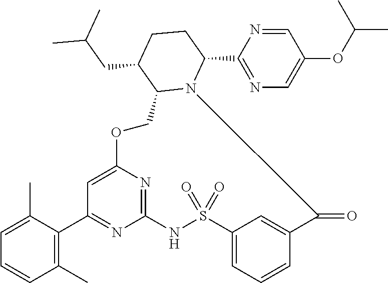 [SMILES: Cc1cccc(C)c1-c1cc2nc(n1)NS(=O)(=O)c1cccc(c1)C(=O)N1[C@@H](c3ncc(OC(C)C)cn3)CC[C@@H](CC(C)C)[C@H]1CO2] | | | +++ | +++ |
| 415 | 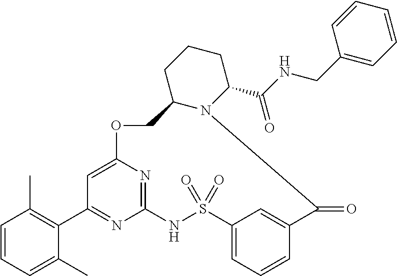 [SMILES: Cc1cccc(C)c1-c1cc2nc(n1)NS(=O)(=O)c1cccc(c1)C(=O)N1[C@H](CCC[C@@H]1C(=O)NCc1ccccc1)CO2] | | | | ++ |
| 416 | 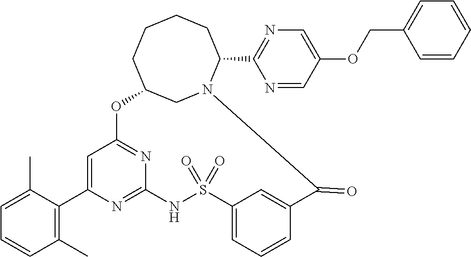 [SMILES: Cc1cccc(C)c1-c1cc2nc(n1)NS(=O)(=O)c1cccc(c1)C(=O)N1C[C@@H](CCCC[C@@H]1c1ncc(OCc3ccccc3)cn1)O2] | | | | ++ |
| 417 | 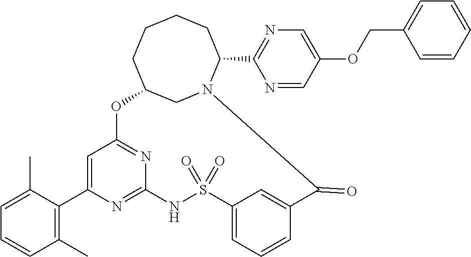 [SMILES: Cc1cccc(C)c1-c1cc2nc(n1)NS(=O)(=O)c1cccc(c1)C(=O)N1C[C@@H](CCCC[C@@H]1c1ncc(OCc3ccccc3)cn1)O2] | | | | + |
| 418 | 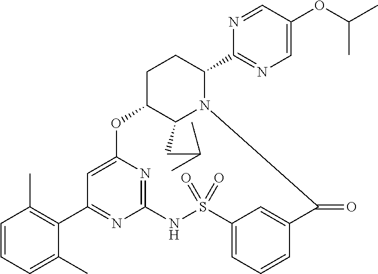 [SMILES: Cc1cccc(C)c1-c1cc2nc(n1)NS(=O)(=O)c1cccc(c1)C(=O)N1[C@@H](c3ncc(OC(C)C)cn3)CC[C@@H](O2)[C@H]1CC(C)C] | | | | + |
| 394 | 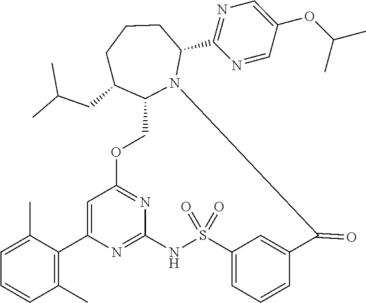 [SMILES: Cc1cccc(C)c1-c1cc2nc(n1)NS(=O)(=O)c1cccc(c1)C(=O)N1[C@@H](c3ncc(OC(C)C)cn3)CCC[C@@H](CC(C)C)[C@H]1CO2] | | | | +++ |
| 395 | 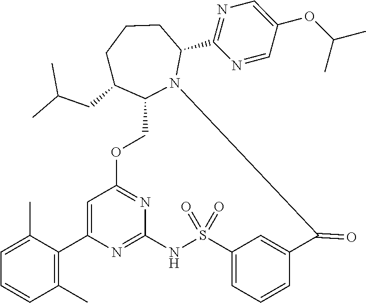 [SMILES: Cc1cccc(C)c1-c1cc2nc(n1)NS(=O)(=O)c1cccc(c1)C(=O)N1[C@@H](c3ncc(OC(C)C)cn3)CCC[C@@H](CC(C)C)[C@H]1CO2] | | | | ++ |
| 419 | 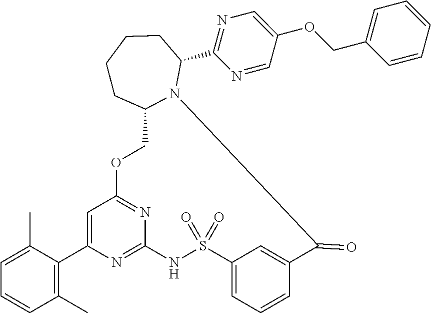 [SMILES: Cc1cccc(C)c1-c1cc2nc(n1)NS(=O)(=O)c1cccc(c1)C(=O)N1[C@@H](CCCC[C@@H]1c1ncc(OCc3ccccc3)cn1)CO2] | | | | ++ |
| 420 | 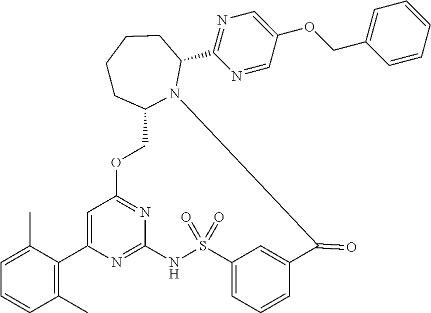 [SMILES: Cc1cccc(C)c1-c1cc2nc(n1)NS(=O)(=O)c1cccc(c1)C(=O)N1[C@@H](CCCC[C@@H]1c1ncc(OCc3ccccc3)cn1)CO2] | | | | + |
| 404 | 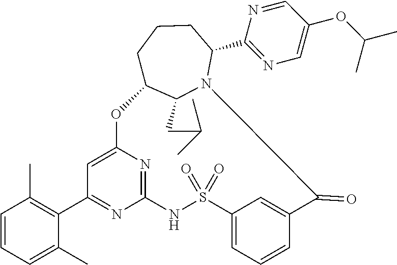 [SMILES: Cc1cccc(C)c1-c1cc2nc(n1)NS(=O)(=O)c1cccc(c1)C(=O)N1[C@@H](c3ncc(OC(C)C)cn3)CCC[C@@H](O2)[C@H]1CC(C)C] | | | | ++ |
| 398 | 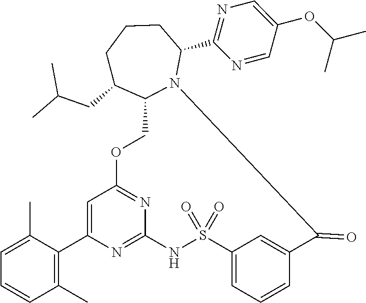 [SMILES: Cc1cccc(C)c1-c1cc2nc(n1)NS(=O)(=O)c1cccc(c1)C(=O)N1[C@@H](c3ncc(OC(C)C)cn3)CCC[C@@H](CC(C)C)[C@H]1CO2] | | | | +++ |
| 399 | 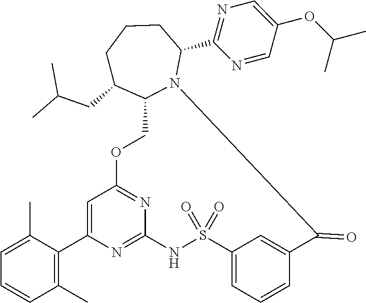 [SMILES: Cc1cccc(C)c1-c1cc2nc(n1)NS(=O)(=O)c1cccc(c1)C(=O)N1[C@@H](c3ncc(OC(C)C)cn3)CCC[C@@H](CC(C)C)[C@H]1CO2] | | | | + |
| 396 | 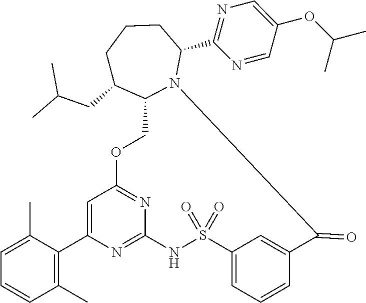 [SMILES: Cc1cccc(C)c1-c1cc2nc(n1)NS(=O)(=O)c1cccc(c1)C(=O)N1[C@@H](c3ncc(OC(C)C)cn3)CCC[C@@H](CC(C)C)[C@H]1CO2] | | | | + |
| 397 | 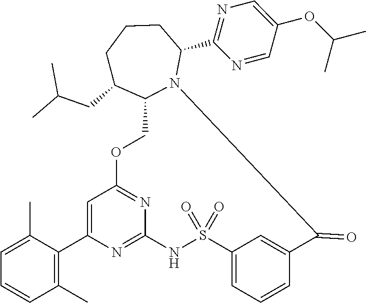 [SMILES: Cc1cccc(C)c1-c1cc2nc(n1)NS(=O)(=O)c1cccc(c1)C(=O)N1[C@@H](c3ncc(OC(C)C)cn3)CCC[C@@H](CC(C)C)[C@H]1CO2] | | | | +++ |
| 406 | 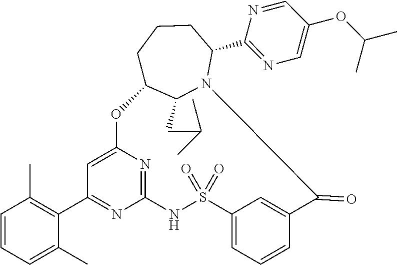 [SMILES: Cc1cccc(C)c1-c1cc2nc(n1)NS(=O)(=O)c1cccc(c1)C(=O)N1[C@@H](c3ncc(OC(C)C)cn3)CCC[C@@H](O2)[C@H]1CC(C)C] | | | | +++ |
| 407 | 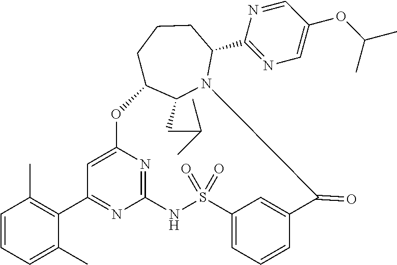 [SMILES: Cc1cccc(C)c1-c1cc2nc(n1)NS(=O)(=O)c1cccc(c1)C(=O)N1[C@@H](c3ncc(OC(C)C)cn3)CCC[C@@H](O2)[C@H]1CC(C)C] | | | | + |
| 421 | 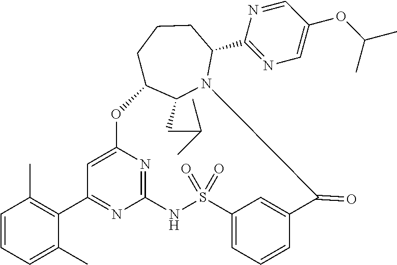 [SMILES: Cc1cccc(C)c1-c1cc2nc(n1)NS(=O)(=O)c1cccc(c1)C(=O)N1[C@@H](c3ncc(OC(C)C)cn3)CCC[C@@H](O2)[C@H]1CC(C)C] | | | | +++ |
| 422 | 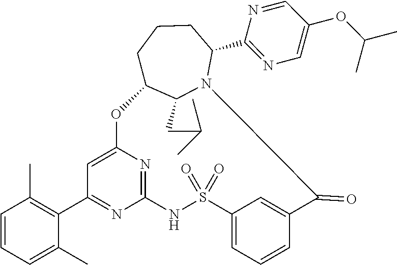 [SMILES: Cc1cccc(C)c1-c1cc2nc(n1)NS(=O)(=O)c1cccc(c1)C(=O)N1[C@@H](c3ncc(OC(C)C)cn3)CCC[C@@H](O2)[C@H]1CC(C)C] | | | | + |
| 423 | 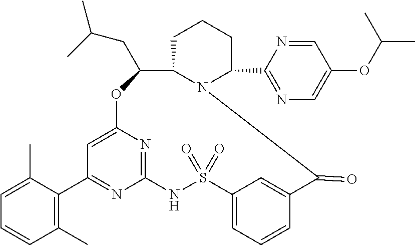 [SMILES: Cc1cccc(C)c1-c1cc2nc(n1)NS(=O)(=O)c1cccc(c1)C(=O)N1[C@@H](c3ncc(OC(C)C)cn3)CCC[C@H]1[C@H](CC(C)C)O2] | | | | ++ |
| 424 | 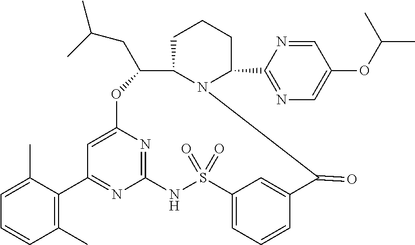 [SMILES: Cc1cccc(C)c1-c1cc2nc(n1)NS(=O)(=O)c1cccc(c1)C(=O)N1[C@@H](c3ncc(OC(C)C)cn3)CCC[C@H]1[C@@H](CC(C)C)O2] | | | | ++ |
| 408 | 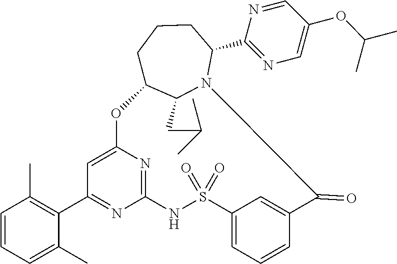 [SMILES: Cc1cccc(C)c1-c1cc2nc(n1)NS(=O)(=O)c1cccc(c1)C(=O)N1[C@@H](c3ncc(OC(C)C)cn3)CCC[C@@H](O2)[C@H]1CC(C)C] | | | | +++ |
| 409 | 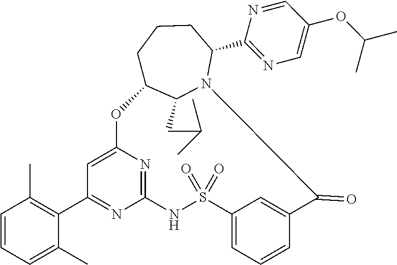 [SMILES: Cc1cccc(C)c1-c1cc2nc(n1)NS(=O)(=O)c1cccc(c1)C(=O)N1[C@@H](c3ncc(OC(C)C)cn3)CCC[C@@H](O2)[C@H]1CC(C)C] | | | | + |
| 425 | 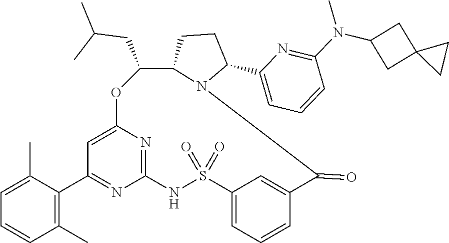 [SMILES: Cc1cccc(C)c1-c1cc2nc(n1)NS(=O)(=O)c1cccc(c1)C(=O)N1[C@@H](c3cccc(N(C)C4CC5(CC5)C4)n3)CC[C@H]1[C@@H](CC(C)C)O2] | | | | ++ |
| 386 | 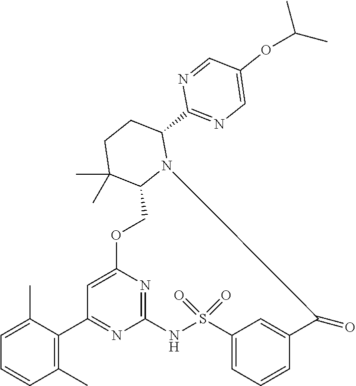 [SMILES: Cc1cccc(C)c1-c1cc2nc(n1)NS(=O)(=O)c1cccc(c1)C(=O)N1[C@@H](c3ncc(OC(C)C)cn3)CCC(C)(C)[C@H]1CO2] | | | | + |
| 405 | 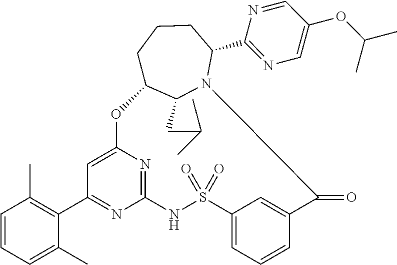 [SMILES: Cc1cccc(C)c1-c1cc2nc(n1)NS(=O)(=O)c1cccc(c1)C(=O)N1[C@@H](c3ncc(OC(C)C)cn3)CCC[C@@H](O2)[C@H]1CC(C)C] | | | | +++ |
| 426 | 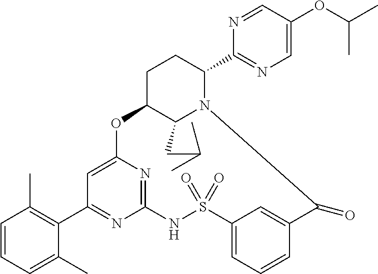 [SMILES: Cc1cccc(C)c1-c1cc2nc(n1)NS(=O)(=O)c1cccc(c1)C(=O)N1[C@@H](c3ncc(OC(C)C)cn3)CC[C@H](O2)[C@H]1CC(C)C] | | | | ++ |
| 400 | 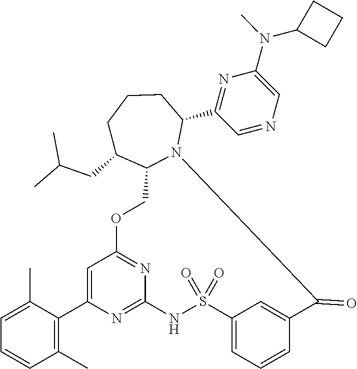 [SMILES: Cc1cccc(C)c1-c1cc2nc(n1)NS(=O)(=O)c1cccc(c1)C(=O)N1[C@@H](c3cncc(N(C)C4CCC4)n3)CCC[C@@H](CC(C)C)[C@H]1CO2] | | | | +++ |
| 401 | 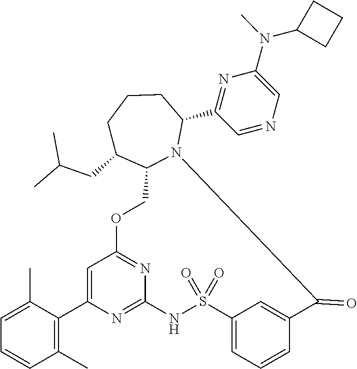 [SMILES: Cc1cccc(C)c1-c1cc2nc(n1)NS(=O)(=O)c1cccc(c1)C(=O)N1[C@@H](c3cncc(N(C)C4CCC4)n3)CCC[C@@H](CC(C)C)[C@H]1CO2] | | | | ++ |
| 387 | 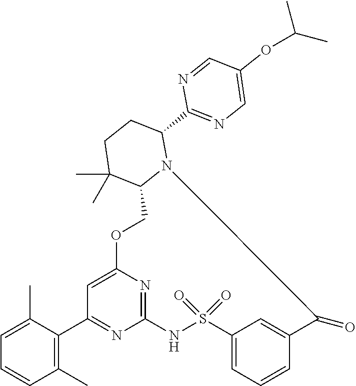 [SMILES: Cc1cccc(C)c1-c1cc2nc(n1)NS(=O)(=O)c1cccc(c1)C(=O)N1[C@@H](c3ncc(OC(C)C)cn3)CCC(C)(C)[C@H]1CO2] | | | | +++ |
| 412 | 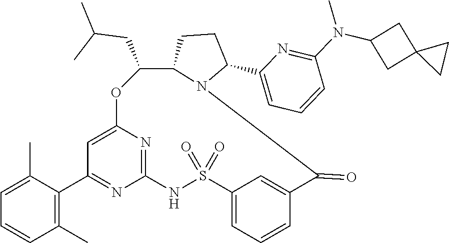 [SMILES: Cc1cccc(C)c1-c1cc2nc(n1)NS(=O)(=O)c1cccc(c1)C(=O)N1[C@@H](c3cccc(N(C)C4CC5(CC5)C4)n3)CC[C@H]1[C@@H](CC(C)C)O2] | | | | ++ |
| 410 | 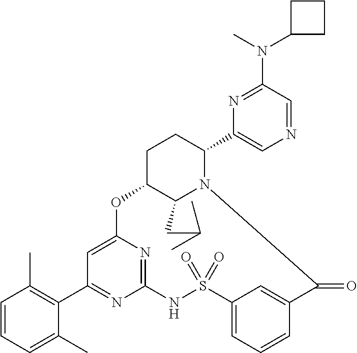 [SMILES: Cc1cccc(C)c1-c1cc2nc(n1)NS(=O)(=O)c1cccc(c1)C(=O)N1[C@@H](c3cncc(N(C)C4CCC4)n3)CC[C@@H](O2)[C@H]1CC(C)C] | | | | +++ |
| 411 | 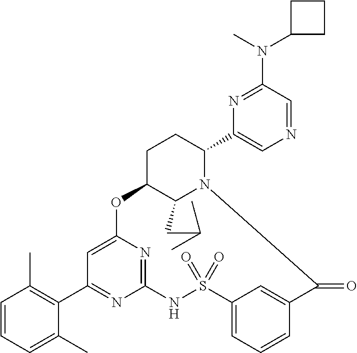 [SMILES: Cc1cccc(C)c1-c1cc2nc(n1)NS(=O)(=O)c1cccc(c1)C(=O)N1[C@@H](c3cncc(N(C)C4CCC4)n3)CC[C@H](O2)[C@H]1CC(C)C] | | | | ++ |
| 402 | 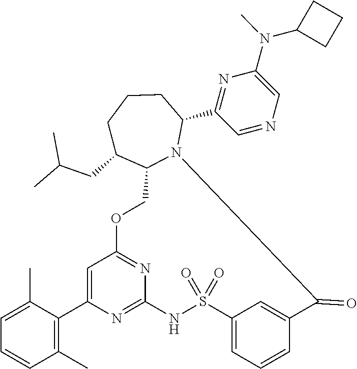 [SMILES: Cc1cccc(C)c1-c1cc2nc(n1)NS(=O)(=O)c1cccc(c1)C(=O)N1[C@@H](c3cncc(N(C)C4CCC4)n3)CCC[C@@H](CC(C)C)[C@H]1CO2] | | | | +++ |
| 403 | 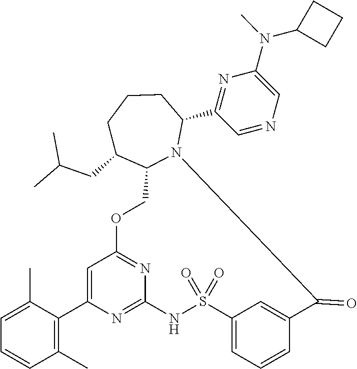 [SMILES: Cc1cccc(C)c1-c1cc2nc(n1)NS(=O)(=O)c1cccc(c1)C(=O)N1[C@@H](c3cncc(N(C)C4CCC4)n3)CCC[C@@H](CC(C)C)[C@H]1CO2] | | | | |

### VIII. Synthesis of (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol

### A. General Methods

Reagents and starting materials were obtained by commercial sources unless otherwise stated and were used without purification.

Proton and carbon NMR spectra were acquired on either a Bruker Biospin DRX 400 MHz FTNMR spectrometer operating at a ¹H and ¹³C resonant frequency of 400 and 100 MHz respectively, or on a 300 MHz NMR spectrometer. One dimensional proton and carbon spectra were acquired using a broadband observe (BBFO) probe with 20 Hz sample rotation at 0.1834 and 0.9083 Hz/Pt digital resolution respectively. All proton and carbon spectra were acquired with temperature control at 30° C. using standard, previously published pulse sequences and routine processing parameters.

NMR (1D & 2D) spectra were also recorded on a Bruker AVNEO 400 MHz spectrometer operating at 400 MHz and 100 MHz respectively equipped with a 5 mm multinuclear Iprobe.

NMR spectra were also recorded on a Varian Mercury NMR instrument at 300 MHz for ¹H using a 45 degree pulse angle, a spectral width of 4800 Hz and 28860 points of acquisition. FID were zero-filled to 32 k points and a line broadening of 0.3 Hz was applied before Fourier transform. ¹⁹F NMR spectra were recorded at 282 MHz using a 30 degree pulse angle, a spectral width of 100 kHz and 59202 points were acquired. FID were zero-filled to 64 k points and a line broadening of 0.5 Hz was applied before Fourier transform.

NMR spectra were also recorded on a Bruker Avance III HD NMR instrument at 400 MHz for ¹H using a 30 degree pulse angle, a spectral width of 8000 Hz and 128 k points of acquisition. FID were zero-filled to 256 k points and a line broadening of 0.3 Hz was applied before Fourier transform. ¹⁹F NMR spectra were recorded at 377 MHz using a 30 deg pulse angle, a spectral width of 89286 Hz and 128 k points were acquired. FID were zero-filled to 256 k points and a line broadening of 0.3 Hz was applied before Fourier transform.

NMR spectra were also recorded on a Bruker AC 250 MHz instrument equipped with a: 5 mm QNP(H1/C₁₃/F19/P31) probe (type: 250-SB, s #23055/0020) or on a Varian 500 MHz instrument equipped with a ID PFG, 5 mm, 50-202/500 MHz probe (model/part #99337300).

Unless stated to the contrary in the following examples, final purity of compounds was determined by reversed phase UPLC using an Acquity UPLC BEH C₁₈ column (50×2.1 mm, 1.7 m particle) made by Waters (pn: 186002350), and a dual gradient run from 1-99% mobile phase B over 3.0 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C. Final purity was calculated by averaging the area under the curve (AUC) of two UV traces (220 nm, 254 nm). Low-resolution mass spectra were reported as [M+1]⁺ species obtained using a single quadrupole mass spectrometer equipped with an electrospray ionization (ESI) source capable of achieving a mass accuracy of 0.1 Da and a minimum resolution of 1000 (no units on resolution) across the detection range.

Solid-state NMR (SSNMR) spectra were recorded on a Bruker-Biospin 400 MHz wide-bore spectrometer equipped with Bruker-Biospin 4 mm HFX probe. Samples were packed into 4 mm ZrO₂ rotors and spun under Magic Angle Spinning (MAS) condition with spinning speed typically set to 12.5 kHz. The proton relaxation time was measured using ¹H MAS Ti saturation recovery relaxation experiment in order to set up proper recycle delay of the ¹³C cross-polarization (CP) MAS experiment. The fluorine relaxation time was measured using ¹⁹F MAS Ti saturation recovery relaxation experiment in order to set up proper recycle delay of the ¹⁹F MAS experiment. The CP contact time of carbon CPMAS experiment was set to 2 ms. A CP proton pulse with linear ramp (from 50% to 100%) was employed. The carbon Hartmann-Hahn match was optimized on external reference sample (glycine). Both carbon and fluorine spectra were recorded with proton decoupling using TPPM15 decoupling sequence with the field strength of approximately 100 kHz.

### B. Procedures for the Synthesis of Intermediates

### Intermediate 1: Preparation of methyl 3-[bis(tert-butoxycarbonyl)amino]-6-bromo-5-(trifluoromethyl)pyridine-2-carboxylate

### Step 1: Methyl 3-(benzhydrylideneamino)-5-(trifluoromethyl)pyridine-2-carboxylate

A mixture of methyl 3-chloro-5-(trifluoromethyl)pyridine-2-carboxylate (47.3 g, 197.43 mmol), diphenylmethanimine (47 g, 259.33 mmol), Xantphos (9.07 g, 15.675 mmol), and cesium carbonate (131 g, 402.06 mmol) in dioxane (800 mL) was degassed with bubbling nitrogen for 30 minutes. Pd(OAc)₂ (3.52 g, 15.679 mmol) was added and the system was purged with nitrogen three times. The reaction mixture was heated at 100° C. for 18 hours. The reaction was cooled to room temperature and filtered on a pad of Celite. The cake was washed with EtOAc and solvents were evaporated under reduced pressure to give methyl 3-(benzhydrylideneamino)-5-(trifluoromethyl)pyridine-2-carboxylate (90 g, 84%) as yellow solid. ESI-MS m/z calc. 384.10855, found 385.1 (M+1)⁺; Retention time: 2.24 minutes. LCMS Method: Kinetex C₁₈ 4.6×50 mm 2.6 μM, 2.0 mL/min, 95% H₂O (0.1% formic acid)+5% acetonitrile (0.1% formic acid) to 95% acetonitrile (0.1% formic acid) gradient (2.0 min) then held at 95% acetonitrile (0.1% formic acid) for 1.0 minute.

### Step 2: Methyl 3-amino-5-(trifluoromethyl)pyridine-2-carboxylate

To a suspension of methyl 3-(benzhydrylideneamino)-5-(trifluoromethyl)pyridine-2-carboxylate (65 g, 124.30 mmol) in methanol (200 mL) was added HCl (3 M in methanol) (146 mL of 3 M, 438.00 mmol). The mixture was stirred at room temperature for 1.5 hours, then the solvent was removed under reduced pressure. The residue was taken up in ethyl acetate (2 L) and dichloromethane (500 mL). The organic phase was washed with 5% aqueous sodium bicarbonate solution (3×500 mL) and brine (2×500 mL), dried over anhydrous sodium sulfate, filtered and the solvent was removed under reduced pressure. The residue was triturated with heptanes (2×50 mL), and the mother liquors were discarded. The solid obtained was triturated with a mixture of dichloromethane and heptanes (1:1, 40 mL) and filtered to afford methyl 3-amino-5-(trifluoromethyl)pyridine-2-carboxylate (25.25 g, 91%) as yellow solid. ¹H NMR (300 MHz, CDCl₃) δ 8.24 (s, 1H), 7.28 (s, 1H), 5.98 (br. s, 2H), 4.00 (s, 3H) ppm. 19F NMR (282 MHz, CDCl₃) δ −63.23 (s, 3F) ppm. ESI-MS m/z calc. 220.046, found 221.1 (M+1)⁺; Retention time: 1.62 minutes. LCMS Method: Kinetex Polar C₁₈ 3.0×50 mm 2.6 μm, 3 min, 5-95% acetonitrile in H₂O (0.1% formic acid) 1.2 mL/min.

### Step 3: Methyl 3-amino-6-bromo-5-(trifluoromethyl)pyridine-2-carboxylate

To a solution of methyl 3-amino-5-(trifluoromethyl)pyridine-2-carboxylate (18.75 g, 80.91 mmol) in acetonitrile (300 mL) at 0° C. was added portion wise N-bromosuccinimide (18.7 g, 105.3 mmol). The mixture was stirred overnight at 25° C. Ethyl acetate (1000 mL) was added. The organic layer was washed with 10% sodium thiosulfate solution (3×200 mL) which was back extracted with ethyl acetate (2×200 mL). The combined organic extracts were washed with saturated sodium bicarbonate solution (3×200 mL), brine (200 mL), dried over sodium sulfate and concentrated in vacuo to provide methyl 3-amino-6-bromo-5-(trifluoromethyl)pyridine-2-carboxylate (25.46 g, 98%). ¹H NMR (300 MHz, CDCl₃) δ 3.93-4.03 (m, 3H), 6.01 (br. s., 2H), 7.37 (s, 1H) ppm. ¹⁹F NMR (282 MHz, CDCl₃) ppm −64.2 (s, 3F). ESI-MS m/z calc. 297.9565, found 299.0 (M+1)⁺; Retention time: 2.55 minutes. LCMS Method: Kinetex C₁₈ 4.6×50 mm 2.6 μM. Temp: 45° C., Flow: 2.0 mL/min, Run Time: 6 minutes. Mobile Phase: Initial 95% H₂O (0.1% formic acid) and 5% acetonitrile (0.1% formic acid) linear gradient to 95% acetonitrile (0.1% formic acid) for 4.0 minutes, then held at 95% acetonitrile (0.1% formic acid) for 2.0 minutes.

### Step 4: Methyl 3-[bis(tert-butoxycarbonyl)amino]-6-bromo-5-(trifluoro methyl)pyridine-2-carboxylate

A mixture of methyl 3-amino-6-bromo-5-(trifluoromethyl)pyridine-2-carboxylate (5 g, 15.549 mmol), (Boc)₂O (11 g, 11.579 mL, 50.402 mmol), DMAP (310 mg, 2.5375 mmol) and CH₂Cl₂ (150 mL) was stirred at room temperature overnight. The reaction mixture was concentrated under reduced pressure, and purification by silica gel chromatography (0-15% ethyl acetate in heptane) provided methyl 3-[bis(tert-butoxycarbonyl)amino]-6-bromo-5-(trifluoromethyl)pyridine-2-carboxylate (6.73 g, 87%) as light yellow solid. ¹H NMR (300 MHz, CDCl₃) δ 1.42 (s, 18H), 3.96 (s, 3H), 7.85 (s, 1H) ppm. ¹⁹F NMR (282 MHz, CDCl₃) δ −63.9 (s, 3F) ppm. ESI-MS m/z calc. 498.06134, Retention time: 2.34 minutes. LCMS Method: Kinetex C₁₈ 4.6×50 mm 2.6 μM. Temp: 45° C., Flow: 2.0 mL/min, Run Time: 3 minutes. Mobile Phase: Initial 95% H₂O (0.1% formic acid) and 5% acetonitrile (0.1% formic acid) linear gradient to 95% acetonitrile (0.1% formic acid) for 2.0 minutes, then held at 95% acetonitrile (0.1% formic acid) for 1.0 minute.

### Intermediate 2: Preparation of 6-bromo-3-(tert-butoxycarbonylamino)-5-(trifluoromethyl)pyridine-2-carboxylic acid

### Step 1: 6-Bromo-3-(tert-butoxycarbonylamino)-5-(trifluoromethyl)pyridine-2-carboxylic acid

To a mixture of methyl 3-[bis(tert-butoxycarbonyl)amino]-6-bromo-5-(trifluoromethyl)pyridine-2-carboxylate (247 g, 494.7 mmol) in THE (1.0 L) was added a solution of LiOH (47.2 g, 1.971 mol) in water (500 mL). The mixture was stirred at ambient temperature for 18 hours, affording a yellow slurry. The mixture was cooled with an ice-bath and slowly acidified with HCl (1000 mL of 2 M, 2.000 mol) keeping the reaction temperature<15° C. The mixture was diluted with heptane (1.5 L), mixed and the organic phase separated. The aqueous phase was extracted with heptane (500 mL). The combined organic phases were washed with brine, dried over MgSO4, filtered and concentrated in vacuo. The crude oil was dissolved in heptane (600 mL), seeded and stirred at ambient temperature for 18 hours, affording a thick slurry. The slurry was diluted with cold heptane (500 mL) and the precipitate collected using a medium frit. The filter cake was washed with cold heptane and air dried for 1 hour, then in vacuo at 45° C. for 48 hours to afford 6-bromo-3-(tert-butoxycarbonylamino)-5-(trifluoromethyl)pyridine-2-carboxylic acid (158.3 g, 83%). ¹H NMR (400 MHz, DMSO-d₆) δ 10.38 (s, 1H), 9.01 (s, 1H), 1.50 (s, 9H) ppm. ESI-MS m/z calc. 383.99326, found 384.9 (M+1)⁺; Retention time: 2.55 minutes. LCMS Method Detail: Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=H₂O (0.05% CF₃CO₂H). Mobile phase B=acetonitrile (0.035% CF₃CO₂H). Flow rate=1.2 mL/min, injection volume=1.5 μL, and column temperature=60° C.

### Intermediate 3: Preparation of 2-Benzyloxy-2-(trifluoromethyl)hex-5-enoic acid

### Step 1: Ethyl 2-hydroxy-2-(trifluoromethyl)hex-5-enoate

To a solution of ethyl 3,3,3-trifluoro-2-oxo-propanoate (25.15 g, 147.87 mmol) in Et20 (270 mL) at −78° C. was added bromo(but-3-enyl)magnesium in THE (190 mL of 0.817 M, 155.23 mmol) dropwise over a period of 1.5 hours (inner temperature −72° C. to −76° C.). The mixture was stirred at −78° C. for 20 minutes. The dry ice-acetone bath was removed. The mixture was slowly warm to 5° C. over 1 hour, added to a mixture of 1 N aqueous HCl (170 mL) and crushed ice (150 g) (pH=4). The two layers were separated. The organic layer was concentrated, and the residue was combined with aqueous phase and extracted with EtOAc (2×150 mL). The combined organic phase was washed with 5% aqueous NaHCO₃(50 mL) and brine (20 mL), and dried with Na₂SO₄. The mixture was filtered and concentrated, and co-evaporated with THE (2×40 mL) to give ethyl 2-hydroxy-2-(trifluoromethyl)hex-5-enoate (37.44 g, 96%) as colorless oil. ¹H NMR (300 MHz, CDCl₃) δ 5.77 (ddt, J=17.0, 10.4, 6.4 Hz, 1H), 5.15-4.93 (m, 2H), 4.49-4.28 (m, 2H), 3.88 (s, 1H), 2.35-2.19 (m, 1H), 2.17-1.89 (m, 3H), 1.34 (t, J=7.0 Hz, 3H) ppm. ¹⁹F NMR (282 MHz, CDCl₃) δ −78.74 (s, 3F) ppm.

### Step 2: Ethyl 2-benzyloxy-2-(trifluoromethyl)hex-5-enoate

To a solution of ethyl 2-hydroxy-2-(trifluoromethyl)hex-5-enoate (24.29 g, 87.6% purity, 94.070 mmol) in DMF (120 mL) at 0° C. was added NaH (60% in mineral oil, 5.64 g, 141.01 mmol) portion-wise. The mixture was stirred at 0° C. for 10 minutes. Benzyl bromide (24.13 g, 141.08 mmol) and TBAI (8.68 g, 23.500 mmol) were added. The mixture was stirred at room temperature overnight. NH₄Cl (3 g, 0.6 eq) was added. The mixture was stirred for 10 minutes. 30 mL of EtOAc was added, then ice-water was added (400 g). The mixture was extracted with CH₂Cl₂ and the combined organic layers were concentrated. Purification by silica gel chromatography (0-20% CH₂Cl₂ in heptanes) provided ethyl 2-benzyloxy-2-(trifluoromethyl)hex-5-enoate (26.05 g, 88%) as pink oil. ¹H NMR (300 MHz, CDCl₃) δ 1.34 (t, J=7.2 Hz, 3H), 2.00-2.19 (m, 3H), 2.22-2.38 (m, 1H), 4.33 (q, J=7.2 Hz, 2H), 4.64 (d, J=10.6 Hz, 1H), 4.84 (d, J=10.9 Hz, 1H), 4.91-5.11 (m, 2H), 5.62-5.90 (m, 1H), 7.36 (s, 5H) ppm. 19F NMR (282 MHz, CDCl₃) δ −70.5 (s, 3F) ppm. ESI-MS m/z calc. 316.12863, found 317.1 (M+1)⁺; Retention time: 2.47 minutes. LCMS Method: Kinetex C₁₈ 4.6×50 mm 2.6 μM. Temp: 45° C., Flow: 2.0 mL/min, Run Time: 3 minutes. Mobile Phase: Initial 95% H₂O (0.1% formic acid) and 5% acetonitrile (0.1% formic acid) linear gradient to 95% acetonitrile (0.1% formic acid) for 2.0 minutes, then held at 95% acetonitrile (0.1% formic acid) for 1.0 minute.

### Step 3: 2-Benzyloxy-2-(trifluoromethyl)hex-5-enoic acid

A solution of sodium hydroxide (7.86 g, 196.51 mmol) in water (60 mL) was added to a solution of ethyl 2-benzyloxy-2-(trifluoromethyl)hex-5-enoate (24.86 g, 78.593 mmol) in methanol (210 mL). The reaction was heated at 50° C. overnight. The reaction was concentrated to remove methanol, diluted with water (150 mL) and the carboxylate sodium salt was washed with heptane (1×100 mL). The aqueous solution was acidified to pH=2 with aqueous 3N solution of HCl. The carboxylic acid was extracted with dichloromethane (3×100 mL) and dried over sodium sulfate. The solution was filtered and concentrated to give 2-benzyloxy-2-(trifluoromethyl)hex-5-enoic acid (22.57 g, 97%) as pale yellow oil. ¹H NMR (300 MHz, DMSO-d₆) δ 14.31 (br. s., 1H), 7.55-7.20 (m, 5H), 5.93-5.70 (m, 1H), 5.17-4.91 (m, 2H), 4.85-4.68 (m, 1H), 4.67-4.55 (m, 1H), 2.32-1.94 (m, 4H) ppm. ¹⁹F NMR (282 MHz, DMSO-d₆) δ −70.29 (s, 3F) ppm. ESI-MS m/z calc. 288.09732, found 287.1 (M−1); Retention time: 3.1 minutes. LCMS Method: Kinetex Polar C₁₈ 3.0×50 mm 2.6 μm, 6 min, 5-95% acetonitrile in H₂O (0.1% formic acid) 1.2 mL/min.

### Intermediate 4: Preparation of (2R)-2-benzyloxy-2-(trifluoromethyl)hex-5-enoic acid

### Step-1: (2R)-2-Benzyloxy-2-(trifluoromethyl)hex-5-enoic acid; (R)-4-quinolyl-[(2S,4S)-5-vinylquinuclidin-2-yl]methanol

To a N₂ purged jacketed reactor set to 20° C. was added isopropyl acetate (IPAC, 100 L, 0.173 M, 20 Vols), followed by previously melted 2-benzyloxy-2-(trifluoromethyl)hex-5-enoic acid (5.00 kg, 17.345 mol) and cinchonidine (2.553 kg, 8.67 mol) made into a slurry with minor amount of the reaction solvent. The reactor was set to ramp internal temperature to 80° C. over 1 hour, with solids going in solution upon heating to set temperature, then the solution was held at temperature for at least 10 minutes, then cooled to 70° C. held and seeded with chiral salt (50 g, 1.0% by wt). The mixture was stirred for 10 minutes, then ramped to 20° C. internal temperature over 4 hours, then held overnight at 20° C. The mixture was filtered, cake washed with isopropyl acetate (10.0 L, 2.0 vols) and dried under vacuum. The cake was then dried in vacuo (50° C., vacuum) to afford 4.7 kg of salt. The resulting solid salt was returned to the reactor by making a slurry with a portion of isopropyl acetate (94 L, 20 vol based on current salt wt), and pumped into reactor and stirred. The mixture was then heated to 80° C. internal, stirred hot slurry for at least 10 minutes, then ramped to 20° C. over 4-6 hours, then stirred overnight at 20° C. The material was then filtered and the cake washed with isopropyl acetate (9.4 L, 2.0 vol), pulled dry, cake scooped out and dried in vacuo (50° C., vacuum) to afford 3.1 kg of solid. The solid (3.1 kg) and isopropyl acetate (62 L, 20 vol based on salt solid wt) was slurried and added to a reactor, stirred under N2 purge and heated to 80° C. and held at temperature at least 10 minutes, then ramped to 20° C. over 4-6 hours, then stirred overnight. The mixture was filtered, cake washed with isopropyl acetate (6.2 L, 2 vol), pulled dry, scooped out and dried in vacuo (50° C., vac) to afford 2.25 kg of solid salt. The solid (2.25 kg) and isopropyl acetate (45 L, 20 vol based on salt solid wt) was slurried and added to a reactor, stirred under N2 purge and heated to 80° C., held at temperature at least 10 minutes, then ramped to 20° C. over 4-6 hours, then stirred overnight. The mixture was filtered, cake washed with isopropyl acetate (4.5 L, 2 vol), pulled dry, scooped out and dried in vacuo (50° C. to afford (2R)-2-benzyloxy-2-(trifluoromethyl)hex-5-enoic acid; (R)-4-quinolyl-[(2S,4S)-5-vinylquinuclidin-2-yl]methanol (1.886 kg, >98.0% ee) as an off-white to tan solid. Chiral purity was determined by Agilent 1200 HPLC instrument using Phenomenex Lux i-Amylose-3 column (3 μm, 150×4.6 mm) and a dual, isocratic gradient run 30% to 70% mobile phase B over 20.0 minutes. Mobile phase A=H₂O (0.1% CF₃CO₂H). Mobile phase B=MeOH (0.1% CF₃CO₂H). Flow rate=1.0 mL/min, injection volume=2 μL, and column temperature=30° C., sample concentration: 1 mg/mL in 60% acetonitrile/40% water.

### Step 2: (2R)-2-Benzyloxy-2-(trifluoromethyl)hex-5-enoic acid

A suspension of (2R)-2-benzyloxy-2-(trifluoromethyl)hex-5-enoic acid; (R)-4-quinolyl-[(2S,4S)-5-vinylquinuclidin-2-yl]methanol (50 g, 87.931 mmol) in ethyl acetate (500.00 mL) was treated with an aqueous solution of hydrochloric acid (200 mL of 1 M, 200.00 mmol). After stirring for 15 minutes at room temperature, the two phases were separated. The aqueous phase was extracted twice with ethyl acetate (200 mL). The combined organic layer was washed with 1 N HCl (100 mL). The organic layer was dried over sodium sulfate, filtered and concentrated. The material was dried over high vacuum overnight to give (2R)-2-benzyloxy-2-(trifluoromethyl)hex-5-enoic acid (26.18 g, 96%) as pale brown oil. ¹H NMR (400 MHz, CDCl₃) δ 7.46-7.31 (m, 5H), 5.88-5.73 (m, 1H), 5.15-4.99 (m, 2H), 4.88 (d, J=10.3 Hz, 1H), 4.70 (d, J=10.3 Hz, 1H), 2.37-2.12 (m, 4H) ppm. ¹⁹F NMR (377 MHz, CDCl₃) δ −71.63 (br s, 3F) ppm. ESI-MS m/z calc. 288.0973, found 287.0 (M−1)⁻; Retention time: 2.15 minutes. LCMS Method: Kinetex Polar C₁₈ 3.0×50 mm 2.6 μm, 3 min, 5-95% acetonitrile in H₂O (0.1% formic acid) 1.2 mL/min.

### Intermediate 5: Preparation of (2R)-2-benzyloxy-2-(trifluoromethyl)hex-5-enehydrazide

### Step 1: tert-Butyl N-[[(2R)-2-benzyloxy-2-(trifluoromethyl)hex-5-enoyl]amino]carbamate

To a solution of (2R)-2-benzyloxy-2-(trifluoromethyl)hex-5-enoic acid (365 g, 1.266 mol) in DMF (2 L) was added HATU (612 g, 1.610 mol) and DIEA (450 mL, 2.584 mol) and the mixture was stirred at ambient temperature for 10 minutes. To the mixture was added tert-butyl N-aminocarbamate (200 g, 1.513 mol) (slight exotherm upon addition) and the mixture was stirred at ambient temperature for 16 hours. The reaction was poured into ice water (5 L). The resultant precipitate was collected by filtration and washed with water. The solid was dissolved in EtOAc (2 L) and washed with brine. The organic phase was dried over MgSO4, filtered, and concentrated in vacuo. The oil was diluted with EtOAc (500 mL) followed by heptane (3 L) and stirred at ambient temperature for several hours affording a thick slurry. The slurry was diluted with additional heptane and filtered to collect fluffy white solid (343 g). The filtrate was concentrated and purification by silica gel chromatography (0-40% EtOAc/hexanes) provided tert-butyl N-[[(2R)-2-benzyloxy-2-(trifluoromethyl)hex-5-enoyl]amino]carbamate (464 g, 91%, combined with product from crystallization). ESI-MS m/z calc. 402.17664, found 303.0 (M+1-Boc)⁺; Retention time: 2.68 minutes. Final purity was determined by reversed phase UPLC using an Acquity UPLC BEH C₁₈ column (50×2.1 mm, 1.7 m particle) made by Waters (pn: 186002350) and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### Step 2: (2R)-2-Benzyloxy-2-(trifluoromethyl)hex-5-enehydrazide

To a solution of tert-butyl N-[[(2R)-2-benzyloxy-2-(trifluoromethyl)hex-5-enoyl]amino]carbamate (464 g, 1.153 mol) in DCM (1.25 L) and was added HCl (925 mL of 4 M, 3.700 mol) and the mixture stirred at ambient temperature for 20 hours. The mixture was concentrated in vacuo removing most of the DCM. The mixture was diluted with isopropyl acetate (1 L) and basified to pH=6 with NaOH (140 g of 50% w/w, 1.750 mol) in 1 L of ice water. The organic phase was separated and washed with 1 L of brine and the combined aqueous phases were extracted with isopropyl acetate (1 L). The combined organic phases were dried over MgSO4, filtered and concentrated in vacuo affording a dark yellow oil of (2R)-2-benzyloxy-2-(trifluoromethyl)hex-5-enehydrazide (358 g, quant.). ¹H NMR (400 MHz, CDCl₃) δ 8.02 (s, 1H), 7.44-7.29 (m, 5H), 5.81 (ddt, J=16.8, 10.1, 6.4 Hz, 1H), 5.13-4.93 (m, 2H), 4.75 (dd, J=10.5, 1.5 Hz, 1H), 4.61 (d, J=10.5 Hz, 1H), 3.78 (s, 2H), 2.43 (ddd, J=14.3, 11.0, 5.9 Hz, 1H), 2.26-1.95 (m, 3H) ppm. ESI-MS m/z calc. 302.1242, found 303.0 (M+1)⁺; Retention time: 2.0 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### Intermediate 6: Preparation of tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate

### Step 1: tert-Butyl N-[2-[[[(2R)-2-benzyloxy-2-(trifluoromethyl)hex-5-enoyl]amino]carbamoyl]-6-bromo-5-(trifluoromethyl)-3-pyridyl]carbamate

To a mixture of 6-bromo-3-(tert-butoxycarbonylamino)-5-(trifluoromethyl)pyridine-2-carboxylic acid (304 g, 789.3 mmol) and (2R)-2-benzyloxy-2-(trifluoromethyl)hex-5-enehydrazide (270 g, 893.2 mmol) in EtOAc (2.25 L) at ambient temperature was added DIEA (425 mL, 2.440 mol). To the mixture was slowly added T3P (622 g of 50% w/w, 977.4 mmol) using an ice-water bath to keep the temperature<35° C. (temperature rose to 34° C.) and the reaction mixture was stirred at ambient temperature for 18 hours. Additional DIEA (100 mL, 574.1 mmol) and T3P (95 g, 298.6 mmol) were added and stirred at ambient temperature for 2 days. Starting material was still observed and additional T3P (252 g, 792 mmol) was added and stirred for 5 days. The reaction was quenched with the slow addition of water (2.5 L) and the mixture stirred for 30 minutes. The organic phase was separated, and the aqueous phase extracted with EtOAc (2 L). The combined organic phases were washed with brine, dried over MgSO4, filtered and concentrated in vacuo. The crude product was dissolved in MTBE (300 mL) and diluted with heptane (3 L), the mixture stirred at ambient temperature for 12 hours affording a light yellow slurry. The slurry was filtered, and the resultant solid was air dried for 2 hours, then in vacuo at 40° C. for 48 hours. The filtrate was concentrated in vacuo and purified by silica gel chromatography (0-20% EtOAc/hexanes) and combined with material obtained from crystallization providing tert-butyl N-[2-[[[(2R)-2-benzyloxy-2-(trifluoromethyl)hex-5-enoyl]amino]carbamoyl]-6-bromo-5-(trifluoromethyl)-3-pyridyl]carbamate (433 g, 82%). ¹H NMR (400 MHz, DMSO) δ 11.07 (s, 1H), 10.91 (s, 1H), 10.32 (s, 1H), 9.15 (s, 1H), 7.53-7.45 (m, 2H), 7.45-7.28 (m, 3H), 5.87 (ddt, J=17.0, 10.2, 5.1 Hz, 1H), 5.09 (dq, J=17.1, 1.3 Hz, 1H), 5.02 (dd, J=10.3, 1.9 Hz, 1H), 4.84 (q, J=11.3 Hz, 2H), 2.37-2.13 (m, 4H), 1.49 (s, 9H) ppm. ESI-MS m/z calc. 668.1069, found 669.0 (M+1)⁺; Retention time: 3.55 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### Step 2: tert-Butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate

To a solution of tert-butyl N-[2-[[[(2R)-2-benzyloxy-2-(trifluoromethyl)hex-5-enoyl]amino]carbamoyl]-6-bromo-5-(trifluoromethyl)-3-pyridyl]carbamate (240 g, 358.5 mmol) in anhydrous acetonitrile (1.5 L) under nitrogen was added DIEA (230 mL, 1.320 mol) and the orange solution heated to 70° C. To the mixture was added p-toluenesulfonyl chloride (80.5 g, 422.2 mmol) in 3 equal portions over 1 hour. The mixture was stirred at 70° C. for 9 hours then additional p-toluenesulfonyl chloride (6.5 g, 34.09 mmol) was added. The mixture was stirred for a total of 24 hours then allowed to cool to ambient temperature. Acetonitrile was removed in vacuo affording a dark orange oil which was diluted with EtOAc (1.5 L) and water (1.5 L). The organic phase was separated and washed with 500 mL of 1M HCl, 500 mL of brine, dried over MgSO4, filtered and concentrated in vacuo. Purification by silica gel chromatography (0-20% EtOAc/hexanes) provided tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate (200 g, 86%). ¹H NMR (400 MHz, DMSO) δ 10.11 (s, 1H), 9.10 (s, 1H), 7.55-7.48 (m, 2H), 7.47-7.28 (m, 3H), 5.87 (ddt, J=16.7, 10.2, 6.4 Hz, 1H), 5.11 (dt, J=17.2, 1.7 Hz, 1H), 5.01 (dt, J=10.2, 1.5 Hz, 1H), 4.74 (d, J=10.6 Hz, 1H), 4.65 (d, J=10.6 Hz, 1H), 2.55-2.42 (m, 2H), 2.30 (qd, J=11.3, 10.3, 6.9 Hz, 2H), 1.52 (s, 9H) ppm. ESI-MS m/z calc. 650.0963, found 650.0 (M+1)⁺; Retention time: 3.78 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### Intermediate 7: Preparation of tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate

### Step 1: tert-Butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate

To a solution of tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate (222 g, 340.8 mmol) in MTBE (1.333 L) was added DIPEA (65.3 mL, 374.9 mmol) followed DMAP (2.09 g, 17.11 mmol). A solution of di-tert-butyl dicarbonate (111.6 g, 511.3 mmol) in MTBE (250 mL) was added over approximately 8 minutes, and the resulting mixture was stirred for additional 30 minutes. 1 L of water was added and the layers separated. The organic layer was washed with KHSO₄ (886 mL of 0.5 M, 443.0 mmol), 300 mL brine, dried with MgSO4 and most (>95%) of the MTBE was evaporated by rotary evaporation at 45° C., leaving a thick oil. 1.125 L of heptane was added, spun in the 45° C. rotovap bath until dissolved, then evaporated out 325 mL of solvent by rotary evaporation. The rotovap bath temp was allowed to drop to room temperature and product started crystallizing out during the evaporation. Then the flask was placed in a −20° C. freezer overnight. The resultant solid was filtered and washed with cold heptane and dried at room temperature for 3 days to give tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate (240.8 g, 94%). ¹H NMR (400 MHz, Chloroform-d) δ 7.95 (s, 1H), 7.52-7.45 (m, 2H), 7.44-7.36 (m, 2H), 7.36-7.29 (m, 1H), 5.83-5.67 (m, 1H), 5.08-5.00 (m, 1H), 5.00-4.94 (m, 1H), 4.79 (d, J=10.4 Hz, 1H), 4.64 (d, J=10.4 Hz, 1H), 2.57-2.26 (m, 3H), 2.26-2.12 (m, 1H), 1.41 (s, 18H) ppm. ESI-MS m/z calc. 750.14874, found 751.1 (M+1)⁺; Retention time: 3.76 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### Intermediate 8: Preparation of tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate

### Step 1: tert-Butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate

tert-Butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate (280 g, 372.6 mmol) was dissolved in DMSO (1.82 L) (yellow solution) and treated with cesium acetate (215 g, 1.120 mol) under stirring at room temperature. The yellow suspension was heated at 80° C. for 5 hours. The reaction mixture was cooled to room temperature and added to a stirred cold emulsion of water (5.5 L) with 1 kg ammonium chloride dissolved in it and a 1:1 mixture of MTBE and heptane (2 L) (in 20 L). The phases were separated and the organic phase washed with water (3×3 L) and with brine (1×2.5 L). The organic phase was dried with MgSO4, filtered, and concentrated under reduced pressure. The resultant yellow solution was diluted with heptane (˜1 L) and seeded with tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate and stirred on the rotovap at 100 mbar pressure at room temperature for 1.5 hours. The solid mass was stirred mechanically for 2 hours at room temperature, resultant thick fine suspension was filtered, washed with dry ice cold heptane and dried under vacuum at 45° C. with a nitrogen bleed for 16 hours to give tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate (220 g, 85%) as an off white solid. ¹H NMR (400 MHz, DMSO-d₆) δ 13.28 (s, 1H), 8.43 (s, 1H), 7.58-7.26 (m, 5H), 5.85 (ddt, J=16.8, 10.3, 6.5 Hz, 1H), 5.10 (dq, J=17.2, 1.6 Hz, 1H), 5.01 (dq, J=10.2, 1.3 Hz, 1H), 4.76 (d, J=11.0 Hz, 1H), 4.65 (d, J=11.0 Hz, 1H), 2.55 (dd, J=9.6, 5.2 Hz, 2H), 2.23 (td, J=13.2, 10.0, 5.7 Hz, 2H), 1.27 (d, J=3.8 Hz, 18H) ppm. ESI-MS m/z calc. 688.23315, found 689.0 (M+1)⁺; Retention time: 3.32 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### C. Preparation of (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol

### Step 1: tert-Butyl N-[2-[5-[(1R)-1-benzyloxy-1-(trifluoromethyl)pent-4-enyl]-1,3,4-oxadiazol-2-yl]-6-[(1R)-1-methylbut-3-enoxy]-5-(trifluoromethyl)-3-pyridyl]-N-tert-butoxycarbonyl-carbamate

Dissolved tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate (159.3 g, 231.3 mmol) and triphenylphosphine (72.9 g, 277.9 mmol) in toluene (1 L), then added (2S)-pent-4-en-2-ol (28.7 mL, 278.9 mmol). Heated this mixture to 45° C., then added DIAD (58.3 mL, 296.1 mmol) (exotherm) slowly over 40 minutes. For the next approximately 2 hours, the mixture was cooled to room temperature. During this cooling period, after the first 10 minutes, triphenylphosphine (6.07 g, 23.14 mmol) was added. After a further 1 hour, additional triphenylphosphine (3.04 g, 11.59 mmol) was added. After a further 23 minutes, DIAD (2.24 mL, 11.57 mmol) was added. After the ˜2 hour cooling to room temperature period, the mixture was cooled to 15° C., and seed crystals of DIAD-triphenylphosphine oxide complex were added which caused precipitation to occur, then added 1000 mL heptane. Stored the mixture at −20° C. for 3 days. Filtered out and discarded the precipitate and concentrated the filtrate to give a red residue/oil. Dissolved the residue in 613 mL heptane at 45° C., then cooled to 0° C., seeded with DIAD-triphenylphosphine oxide complex, stirred at 0° C. for 30 minutes, then filtered the solution. The filtrate was concentrated to a smaller volume, then loaded onto a 1.5 kg silica gel column (column volume=2400 mL, flow rate=600 mL/min). Ran a gradient of 1% to 6% EtOAc in hexanes over 32 minutes (8 column volumes), then held at 6% EtOAc in hexanes until the product finished eluting which gave tert-butyl N-[2-[5-[(1R)-1-benzyloxy-1-(trifluoromethyl)pent-4-enyl]-1,3,4-oxadiazol-2-yl]-6-[(1R)-1-methylbut-3-enoxy]-5-(trifluoromethyl)-3-pyridyl]-N-tert-butoxycarbonyl-carbamate (163.5 g, 93%). ¹H NMR (400 MHz, Chloroform-d) δ 7.82 (s, 1H), 7.43-7.27 (m, 5H), 5.88-5.69 (m, 2H), 5.35 (h, J=6.2 Hz, 1H), 5.16-4.94 (m, 4H), 4.81 (d, J=10.7 Hz, 1H), 4.63 (d, J=10.7 Hz, 1H), 2.58-2.15 (m, 6H), 1.42 (s, 18H), 1.36 (d, J=6.2 Hz, 3H) ppm. ESI-MS m/z calc. 756.2958, found 757.3 (M+1)⁺; Retention time: 4.0 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=water (0.05% CF₃CO₂H). Mobile phase B=acetonitrile (0.035% CF₃CO₂H). Flow rate=1.2 mL/min, injection volume=1.5 μL, and column temperature=60° C.

### Step 2: tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]-N-tert-butoxycarbonyl-carbamate (E/Z mixture)

The following reaction was run, split equally between two, 12 L reaction flasks run in parallel. Mechanical stirring was employed, and reactions were subjected to a constant nitrogen gas purge using a coarse porosity gas dispersion tube. To each flask was added tert-butyl N-[2-[5-[(1R)-1-benzyloxy-1-(trifluoromethyl)pent-4-enyl]-1,3,4-oxadiazol-2-yl]-6-[(1R)-1-methylbut-3-enoxy]-5-(trifluoromethyl)-3-pyridyl]-N-tert-butoxycarbonyl-carbamate (54 g, 71.36 mmol in each flask) dissolved in DCE (8 L in each flask) and both flasks were strongly purged with nitrogen at room temperature. Both flasks were heated to 62° C. and Grubbs 1^{st} Generation Catalyst (9 g, 10.94 mmol in each flask) was added to each reaction and stirred at 400 rpm while setting an internal temperature control to 75° C. with strong nitrogen purging (both reactions reached ˜75° C. after approximately 20 min). After 5 hours, 15 minutes, the internal temperature control was set to 45° C. After approximately 2 hours, 2-sulfanylpyridine-3-carboxylic acid (11 g, 70.89 mmol in each flask) was added to each flask, followed by triethylamine (10 mL, 71.75 mmol in each flask). On completion of addition, the nitrogen purge was turned off and both reaction flasks were stirred at 45° C. open to air overnight. The reactions were then removed from heat and 130 g of silica gel was added to each reaction and each was stirred at room temperature. After approximately 2 hours, the green mixtures were combined and filtered over Celite then concentrated by rotary evaporation at 43° C. The obtained residue was dissolved in dichloromethane/heptane 1:1 (400 mL) and the formed orange solid was removed by filtration. The greenish mother liquor was evaporated to give 115.5 g of a green foam. Dissolved this material in 500 mL of 1:1 dichloromethane/hexanes then loaded onto a 3 kg silica gel column (column volume=4800 mL, flow rate=900 mL/min). Ran a gradient of 2% to 9% EtOAc in hexanes over 43 minutes (8 column volumes), then ran at 9% EtOAc until the product finished eluting giving 77.8 g of impure product. This material was co-evaporated with methanol (˜500 mL) then diluted with methanol (200 mL) to give 234.5 g of a methanolic solution, which was halved and each half was purified by reverse phase chromatography (3.8 kg C₁₈ column, column volume=3300 mL, flow rate=375 mL/min, loaded as solution in methanol). Ran the column at 55% acetonitrile for ˜5 minutes (0.5 column volumes), then at a gradient of 55% to 100% acetonitrile in water over ˜170 minutes (19-20 column volumes), then held at 100% acetonitrile until the product and impurities finished eluting. Clean product fractions from both columns were combined and concentrated by rotary evaporation then transferred with ethanol into 5 L flask, evaporated and carefully dried (becomes a foam) to give as a mixture of olefin isomers, tert-butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]-N-tert-butoxycarbonyl-carbamate (E/Z mixture) (55.5 g, 53%). ESI-MS m/z calc. 728.26447, found 729.0 (M+1)⁺; Retention time: 3.82 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=water (0.05% CF₃CO₂H). Mobile phase B=acetonitrile (0.035% CF₃CO₂H). Flow rate=1.2 mL/min, injection volume=1.5 μL, and column temperature=60° C.

### Step 3: tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate

tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]-N-tert-butoxycarbonyl-carbamate (E/Z mixture) (11.7 g, 16.06 mmol) was dissolved in stirring ethanol (230 mL) and cycled the flask 3 times vacuum/nitrogen and treated with 10% Pd/C (50% water wet, 2.2 g of 5% w/w, 1.034 mmol). The mixture was cycled 3 times between vacuum/nitrogen and 3 times between vacuum/hydrogen. The mixture was then stirred strongly under hydrogen (balloon) for 7.5 hours. The catalyst was removed by filtration, replaced with fresh 10% Pd/C (50% water wet, 2.2 g of 5% w/w, 1.034 mmol) and stirred vigorously under hydrogen (balloon) overnight. Then, the catalyst was removed again by filtration, the filtrate evaporated and the residue (11.3 g, 1 g set aside) was dissolved in ethanol (230 mL), charged with fresh 10% Pd/C (50% water wet, 2.2 g of 5% w/w, 1.034 mmol) and stirred vigorously under hydrogen (balloon) for 6 hours, recharged again with fresh 10% Pd/C (50% water wet, 2.2 g of 5% w/w, 1.034 mmol) and stirred vigorously under hydrogen (balloon) overnight. The catalyst was removed by filtration and the filtrate was evaporated (10 g of residue obtained). This crude material (10 g+1 g set aside above) was purified by silica gel chromatography (330 g column, liquid load in dichloromethane) with a linear gradient of 0% to 15% ethyl acetate in hexane until the product eluted followed by 15% to 100% ethyl acetate in hexane to giving, as a colorless foam, tert-butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate (9.1 g, 78%). ESI-MS m/z calc. 730.2801, found 731.0 (M+1)⁺; Retention time: 3.89 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=water (0.05% CF₃CO₂H). Mobile phase B=acetonitrile (0.035% CF₃CO₂H). Flow rate=1.2 mL/min, injection volume=1.5 μL, and column temperature=60° C.

### Step 4: (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol

tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate (8.6 g, 11.77 mmol) was dissolved in ethanol (172 mL) then the flask was cycled 3 times between vacuum/nitrogen. Treated the mixture with 10% Pd/C (50% water wet, 1.8 g of 5% w/w, 0.8457 mmol) then cycled 3 times between vacuum/nitrogen and 3 times between vacuum/hydrogen and then stirred vigorously under hydrogen (balloon) at room temperature for 18 hours. The mixture was cycled 3 times between vacuum/nitrogen, filtered over Celite, washing with ethanol, and then the filtrate was evaporated to give 7.3 g of tert-butyl N-tert-butoxycarbonyl-N-[(6R,12R)-6-hydroxy-12-methyl-6,15-bis(trifluoromethyl)-13,19-dioxa-3,4,18-triazatricyclo[12.3.1.12,5]nonadeca-1(18),2,4,14,16-pentaen-17-yl]carbamate an off-white solid. 1H NMR and MS confirmed the expected product. CFTR modulatory activity was confirmed using a standard Ussing Chamber Assay for CFTR potentiator activity.

### OTHER EMBODIMENTS

The foregoing discussion discloses and describes merely exemplary embodiments of this disclosure. One skilled in the art will readily recognize from such discussion and from the accompanying drawings and claims, that various changes, modifications and variations can be made therein without departing from the spirit and scope of this disclosure as defined in the following claims.

1. A compound of Formula I: 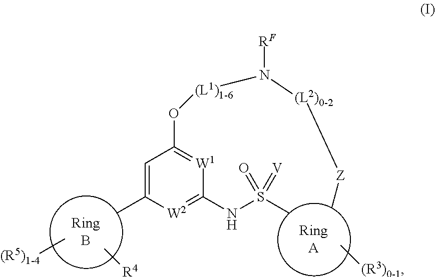
a tautomer thereof, a deuterated derivative of the compound or tautomer, or a pharmaceutically acceptable salt of any of the foregoing, wherein:
Ring A is selected from:
C₆-C₁₀ aryl,
C₃-C₁₀ cycloalkyl,
3- to 10-membered heterocyclyl, and
5- to 10-membered heteroaryl;
Ring B is selected from:
C₆-C₁₀ aryl,
C₃-C₁₀ cycloalkyl,
3- to 10-membered heterocyclyl, and
5- to 10-membered heteroaryl;
V is selected from O and NH;
W¹ is selected from N and CH;
W² is selected from N and CH; provided that at least one of W¹ and W² is N;
Z is selected from O, NR^{ZN}, and C(R^{ZC})₂, provided that when L² is absent, Z is C(R^{ZC})₂;
each L¹ is independently selected from C(R^{L1})₂;
each L² is independently selected from C(R^{L2})₂;
each R³ is independently selected from:
halogen,
C₁-C₆ alkyl,
C₁-C₆ alkoxy,
C₃-C₁₀ cycloalkyl,
C₆-C₁₀ aryl optionally substituted with 1-3 groups independently selected from C₁-C₆ alkyl, and
3- to 10-membered heterocyclyl;
R⁴ is selected from hydrogen and C₁-C₆ alkyl;
each R⁵ is independently selected from:
hydrogen,
halogen,
hydroxyl,
N(R^{N})₂,
—SO-Me,
—CH═C(R^{LC})₂, wherein both R^{LC} are taken together to form a C₃-C₁₀ cycloalkyl,
C₁-C₆ alkyl optionally substituted with 1-3 groups independently selected from:
hydroxyl,
C₁-C₆ alkoxy optionally substituted with 1-3 groups independently selected from C₁-C₆ alkoxy and C₆-C₁₀ aryl,
C₃-C₁₀ cycloalkyl,
—(O)₀₋₁—(C₆-C₁₀ aryl) optionally substituted with 1-3 groups independently selected from C₁-C₆ alkyl and C₁-C₆ alkoxy,
3- to 10-membered heterocyclyl, and
N(R^{N})₂,
C₁-C₆ alkoxy optionally substituted with 1-3 groups independently selected from:
halogen,
C₆-C₁₀ aryl, and
C₃-C₁₀ cycloalkyl optionally substituted with 1-3 groups independently selected from C₁-C₆ fluoroalkyl,
C₁-C₆ fluoroalkyl,
C₃-C₁₀ cycloalkyl,
C₆-C₁₀ aryl, and
3- to 10-membered heterocyclyl;
R^{ZN} is selected from:
hydrogen,
C₁-C₉ alkyl optionally substituted with 1-3 groups independently selected from:
hydroxyl,
oxo,
cyano,
C₁-C₆ alkoxy optionally substituted with 1-3 groups independently selected from halogen and C₁-C₆ alkoxy,
N(R^{N})₂,
SO₂Me,
C₃-C₁₀ cycloalkyl optionally substituted with 1-3 groups independently selected from:
hydroxyl,
C₁-C₆ alkyl optionally substituted with 1-3 groups independently selected from hydroxyl, oxo, C₁-C₆ alkoxy, C₆-C₁₀ aryl, and N(R^{N})₂,
C₁-C₆ fluoroalkyl,
C₁-C₆ alkoxy, and
COOH,
N(R^{N})₂,
C₆-C₁₀ aryl, and
3- to 10-membered heterocyclyl optionally substituted with 1-3 groups independently selected from oxo and C₁-C₆ alkyl,
C₆-C₁₀ aryl optionally substituted with 1-3 groups independently selected from:
halogen,
hydroxyl,
cyano,
SiMe₃,
SO₂Me,
SF₅,
N(R^{N})₂,
P(O)Me₂,
—(O)₀₋₁—(C₃-C₁₀ cycloalkyl) optionally substituted with 1-3 groups independently selected from C₁-C₆ fluoroalkyl,
C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
C₁-C₆ alkoxy optionally substituted with 1-3 groups independently selected from hydroxyl, oxo, N(R^{N})₂, and C₆-C₁₀ aryl,
C₁-C₆ fluoroalkyl,
3- to 10-membered heterocyclyl optionally substituted with 1-3 groups independently selected from C₁-C₆ alkyl,
—(O)₀₋₁—(C₆-C₁₀ aryl), and
—(O)₀₋₁-(5- to 10-heteroaryl) optionally substituted with hydroxyl, oxo, N(R^{N})₂, C₁-C₆ alkyl, C₁-C₆ alkoxy, C₁-C₆ fluoroalkyl, and C₃-C₁₀ cycloalkyl,
3- to 10-membered heterocyclyl optionally substituted with 1-4 groups independently selected from:
hydroxyl,
oxo,
N(R^{N})₂,
C₁-C₆ alkyl (optionally substituted with 1-3 groups independently selected from oxo and C₁-C₆ alkoxy),
C₁-C₆ alkoxy,
C₁-C₆ fluoroalkyl,
C₆-C₁₀ aryl optionally substituted with 1-3 groups independently selected from halogen, and
5- to 10-membered heteroaryl, and
5- to 10-membered heteroaryl optionally substituted with 1-3 groups independently selected from:
hydroxyl,
cyano,
oxo,
halogen,
B(OH)₂,
N(R^{N})₂,
C₁-C₆ alkyl optionally substituted with 1-3 groups independently selected from hydroxyl, oxo, C₁-C₆ alkoxy (optionally substituted with 1-3-SiMe₃), and N(R^{N})₂,
C₁-C₆ alkoxy optionally substituted with 1-3 groups independently selected from hydroxyl, oxo, C₁-C₆ alkoxy, N(R^{N})₂, and C₃-C₁₀ cycloalkyl,
C₁-C₆ fluoroalkyl,
—(O)₀₋₁—(C₃-C₁₀ cycloalkyl) optionally substituted with 1-3 groups independently selected from C₁-C₆ alkyl,
—(O)₀₋₁—(C₆-C₁₀ aryl),
—(O)₀₋₁-(3- to 10-membered heterocyclyl) optionally substituted with 1-4 groups independently selected from hydroxyl, oxo, halogen, cyano, N(R^{N})₂, C₁-C₆ alkyl (optionally substituted with 1-3 groups independently selected from hydroxyl, oxo, N(R^{N})₂, and C₁-C₆ alkoxy), C₁-C₆ alkoxy, C₁-C₆ fluoroalkyl, 3- to 10-membered heterocyclyl (optionally substituted with 1-3 groups independently selected from C₁-C₆ fluoroalkyl) and
5- to 10-membered heteroaryl optionally substituted with 1-4 groups independently selected from C₁-C₆ alkyl and C₃-C₁₀ cycloalkyl,
C₁-C₆ fluoroalkyl,
C₃-C₁₀ cycloalkyl optionally substituted with 1-3 groups independently selected from:
hydroxyl,
oxo,
halogen,
cyano,
N(R^{N})₂,
C₁-C₆ alkyl optionally substituted with 1-3 groups independently selected from:
hydroxyl,
oxo,
N(R^{N})₂,
C₁-C₆ alkoxy, and
C₆-C₁₀ aryl,
C₁-C₆ alkoxy optionally substituted with 1-3 groups independently selected from halogen, oxo, C₆-C₁₀ aryl, and N(R^{N})₂,
halogen,
C₃-C₁₀ cycloalkyl,
3- to 10-member heterocyclyl optionally substituted with 1-3 groups independently selected from C₁-C₆ alkyl, and
5- to 10-membered heteroaryl optionally substituted with 1-3 groups independently selected from:
hydroxyl,
cyano,
oxo,
halogen,
N(R^{N})₂,
C₁-C₆ alkyl optionally substituted with 1-3 groups independently selected from hydroxyl, oxo, C₁-C₆ alkoxy, and N(R^{N})₂,
C₁-C₆ alkoxy optionally substituted with 1-3 groups independently selected from hydroxyl, C₁-C₆ alkoxy, N(R^{N})₂, and C₃-C₁₀ cycloalkyl,
C₁-C₆ fluoroalkyl,
—(O)₀₋₁—(C₃-C₁₀ cycloalkyl) optionally substituted with 1-3 groups independently selected from C₁-C₆ alkyl,
C₆-C₁₀ aryl, and
3- to 10-membered heterocyclyl optionally substituted with 1-3 groups independently selected from C₁-C₆ alkyl,
C₆-C₁₀ aryl,
3- to 10-membered heterocyclyl optionally substituted with 1-3 groups independently selected from:
oxo,
C₁-C₆ alkyl optionally substituted with 1-3 groups independently selected from:
oxo,
hydroxyl,
N(R^{N})₂,
C₁-C₆ alkoxy optionally substituted with 1-3 groups independently selected from halogen and C₆-C₁₀ aryl, and
—(O)₀₋₁—(C₃-C₁₀ cycloalkyl),
C₁-C₆ fluoroalkyl,
C₃-C₁₀ cycloalkyl optionally substituted with 1-3 groups independently selected from halogen, and
3- to 10-membered heterocyclyl,
5- to 10-membered heteroaryl optionally substituted with 1-3 groups independently selected from:
halogen,
C₁-C₆ alkyl optionally substituted with 1-3 groups independently selected from oxo, C₁-C₆ alkoxy, and N(R^{N})₂, and
3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl), and
R^{F};
each R^{ZC} is independently selected from:
hydrogen,
C₁-C₆ alkyl optionally substituted with 1-3 groups independently selected from C₆-C₁₀ aryl (optionally substituted with 1-3 groups independently selected from C₁-C₆ alkyl),
C₆-C₁₀ aryl optionally substituted with 1-3 groups independently selected from C₁-C₆ alkyl, and
R^{F};
or two R^{ZC} are taken together to form an oxo group;
each R^{L1} is independently selected from:
hydrogen,
N(R^{N})₂, provided that two N(R^{N})₂ are not bonded to the same carbon,
C₁-C₉ alkyl optionally substituted with 1-3 groups independently selected from:
halogen,
hydroxyl,
oxo,
N(R^{N})₂,
C₁-C₆ alkoxy optionally substituted with 1-3 groups independently selected from C₆-C₁₀ aryl,
C₃-C₁₀ cycloalkyl optionally substituted with 1-3 groups independently selected from halogen and C₁-C₆ fluoroalkyl,
C₆-C₁₀ aryl optionally substituted with 1-3 groups independently selected from C₁-C₆ alkyl, and
3- to 10-membered heterocyclyl optionally substituted with 1-3 groups independently selected from C₁-C₆ alkyl (optionally substituted with 1-3 groups independently selected from hydroxyl and oxo),
C₃-C₁₀ cycloalkyl,
C₆-C₁₀ aryl optionally substituted with 1-4 groups independently selected from:
halogen,
cyano,
SiMe₃,
POMe₂,
C₁-C₇ alkyl optionally substituted with 1-3 groups independently selected from:
hydroxyl,
oxo,
cyano,
SiMe₃,
N(R^{N})₂, and
C₃-C₁₀ cycloalkyl optionally substituted with 1-3 groups independently selected from C₁-C₆ fluoroalkyl,
C₁-C₆ alkoxy optionally substituted with 1-3 groups independently selected from:
C₃-C₁₀ cycloalkyl optionally substituted with 1-3 groups independently selected from C₁-C₆ fluoroalkyl, and
C₁-C₆ alkoxy,
C₁-C₆ fluoroalkyl,
C₃-C₁₀ cycloalkyl optionally substituted with 1-3 groups independently selected from C₁-C₆ alkyl and C₁-C₆ fluoroalkyl,
C₆-C₁₀ aryl,
3- to 10-membered heterocyclyl optionally substituted with 1-3 groups independently selected from C₁-C₆ alkyl, and
5- to 10-membered heteroaryl,
3- to 10-membered heterocyclyl optionally substituted with 1-3 groups independently selected from:
C₁-C₆ alkyl optionally substituted with 1-3 groups independently selected from:
oxo, and
C₁-C₆ alkoxy,
5- to 10-membered heteroaryl optionally substituted with 1-3 groups independently selected from:
C₁-C₆ alkyl optionally substituted with 1-3 groups independently selected from:
C₃-C₁₀ cycloalkyl optionally substituted with 1-3 groups independently selected from C₁-C₆ fluoroalkyl, and
C₆-C₁₀ aryl optionally substituted with 1-3 groups independently selected from C₁-C₆ alkyl, and
R^{F};
or two R^{L1} on the same carbon atom are taken together to form an oxo group;
each R^{L2} is independently selected from hydrogen and R^{F}; or two R^{L2} on the same carbon atom are taken together to form an oxo group; provided that at least one R^{L1} or R^{L2} is R^{F};
each R^{N} is independently selected from:
hydrogen,
C₁-C₈ alkyl optionally substituted with 1-3 groups independently selected from:
oxo,
halogen,
hydroxyl,
NH₂,
NHMe,
NMe₂,
C₁-C₆ alkoxy optionally substituted with 1-3 groups independently selected from C₆-C₁₀ aryl,
—(O)₀₋₁—(C₃-C₁₀ cycloalkyl),
C₆-C₁₀ aryl optionally substituted with 1-3 groups independently selected from halogen and C₁-C₆ alkyl,
3- to 14-membered heterocyclyl optionally substituted with 1-4 groups independently selected from oxo and C₁-C₆ alkyl, and
5- to 14-membered heteroaryl optionally substituted with 1-4 groups independently selected from oxo and C₁-C₆ alkyl,
C₃-C₁₀ cycloalkyl optionally substituted with 1-3 groups independently selected from:
hydroxyl,
NH₂, and
NHMe, and
C₁-C₆ alkyl optionally substituted with 1-3 groups independently selected from hydroxyl,
C₆-C₁₀ aryl, and
3- to 10-membered heterocyclyl;
or two R^{N} on the same nitrogen atom are taken together with the nitrogen to which they are bonded to form a 3- to 10-membered heterocyclyl optionally substituted with 1-3 groups selected from:
hydroxyl,
oxo,
cyano,
C₁-C₆ alkyl optionally substituted with 1-3 groups independently selected from oxo, hydroxyl, C₁-C₆ alkoxy, and N(R^{N2})₂, wherein each R^{N2} is independently selected from hydrogen and C₁-C₆ alkyl,
C₁-C₆ alkoxy, and
C₁-C₆ fluoroalkyl;
or one R⁴ and one R^{L1} are taken together to form a C₆-C₈ alkylene;
two R^{F} taken together with the atoms to which they are bonded form a group selected from:
C₃-C₁₀ cycloalkyl optionally substituted with 1-3 groups independently selected from C₁-C₆ alkyl,
C₆-C₁₀ aryl optionally substituted with 1-3 groups independently selected from:
halogen,
C₁-C₆ alkyl,
N(R^{N})₂, and
3- to 10-membered heterocyclyl optionally substituted with 1-3 groups independently selected from hydroxyl,
3- to 11-membered heterocyclyl optionally substituted with 1-3 groups independently selected from:
oxo,
N(R^{N})₂,
C₁-C₉ alkyl optionally substituted with 1-4 groups independently selected from:
oxo,
halogen,
hydroxyl,
N(R^{N})₂,
—SO₂—(C₁-C₆ alkyl),
C₁-C₆ alkoxy optionally substituted with 1-3 groups independently selected from halogen, C₆-C₁₀ aryl,
C₆-C₁₀ aryl optionally substituted with 1-3 groups independently selected from hydroxyl, halogen, cyano, C₁-C₆ alkyl (optionally substituted with 1-3 groups independently selected from oxo and C₁-C₆ alkoxy), C₁-C₆ alkoxy (optionally substituted with 1-3 groups independently selected from C₆-C₁₀ aryl), —(O)₀₋₁—(C₁-C₆ fluoroalkyl), and C₆-C₁₀ aryl (optionally substituted with 1-3 groups independently selected from C₁-C₆ alkoxy),
—(O)₀₋₁—(C₃-C₁₀ cycloalkyl) optionally substituted with 1-4 groups independently selected from hydroxyl, halogen, N(R^{N})₂, C₁-C₆ alkyl (optionally substituted with 1-3 groups independently selected from oxo, hydroxyl, and C₁-C₆ alkoxy), C₁-C₆ fluoroalkyl, and C₆-C₁₀ aryl,
3- to 10-membered heterocyclyl optionally substituted with 1-3 groups independently selected from oxo, C₁-C₆ alkyl (optionally substituted with 1-3 groups independently selected from C₆-C₁₀ aryl (optionally substituted with 1-3 groups independently selected from halogens)), C₁-C₆ alkoxy, C₃-C₁₀ cycloalkyl, and R^{N},
—O-(5- to 12-membered heteroaryl) optionally substituted with 1-3 groups independently selected from C₆-C₁₀ aryl (optionally substituted with 1-3 groups independently selected from halogen) and C₁-C₆ alkyl, and
5- to 10-membered heteroaryl optionally substituted with 1-3 groups independently selected from hydroxyl, oxo, N(R^{N})₂, C₁-C₆ alkyl (optionally substituted with 1-3 groups independently selected from cyano), C₁-C₆ alkoxy, —(O)₀₋₁—(C₁-C₆ fluoroalkyl), —O—(C₆-C₁₀ aryl), and C₃-C₁₀ cycloalkyl,
C₃-C₁₂ cycloalkyl optionally substituted with 1-4 groups independently selected from halogen, C₁-C₆ alkyl, and C₁-C₆ fluoroalkyl,
C₆-C₁₀ aryl,
3- to 10-membered heterocyclyl, and
5- to 10-membered heteroaryl optionally substituted with 1-3 groups independently selected from C₁-C₆ alkoxy, C₁-C₆ fluoroalkyl, and N(R^{N})₂, and
5- to 12-membered heteroaryl optionally substituted with 1-3 groups independently selected from C₁-C₆ alkyl (optionally substituted with C₆-C₁₀ aryl) and C₁-C₆ fluoroalkyl.

2. The compound, tautomer, deuterated derivative, or pharmaceutically acceptable salt according to claim 1, selected from compounds of Formula Ia: 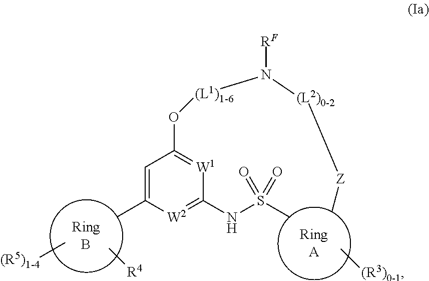
tautomers thereof, deuterated derivatives of the compounds and tautomers, and pharmaceutically acceptable salts of the compounds, tautomers, and deuterated derivatives, wherein Ring A, Ring B, W¹, W², Z, L¹, L², R³, R⁴, R⁵, and R^{F} are defined as according to claim 1.

3. The compound, tautomer, deuterated derivative, or pharmaceutically acceptable salt according to claim 1, selected from compounds of Formula IIa: 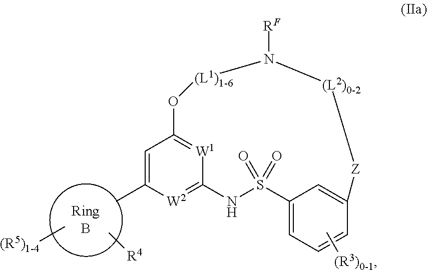
tautomers thereof, deuterated derivatives of the compounds and tautomers, and pharmaceutically acceptable salts of the compounds, tautomers, and deuterated derivatives, wherein Ring B, W¹, W², Z, L¹, L², R³, R⁴, R⁵, and R^{F} are defined as according to claim 1.

4. The compound, tautomer, deuterated derivative, or pharmaceutically acceptable salt according to claim 1, selected from compounds of Formula IIb: 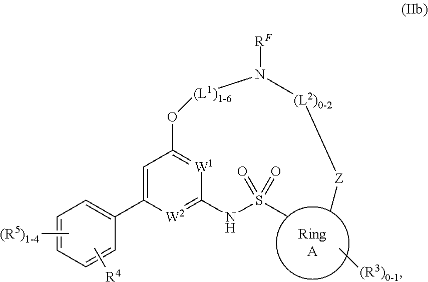
tautomers thereof, deuterated derivatives of the compounds and tautomers, and pharmaceutically acceptable salts of the compounds, tautomers, and deuterated derivatives, wherein Ring A, W¹, W², Z, L¹, L², R³, R⁴, R⁵, and R^{F} are defined as according to claim 1.

5. The compound, tautomer, deuterated derivative, or pharmaceutically acceptable salt according to claim 1, selected from compounds of Formula III: 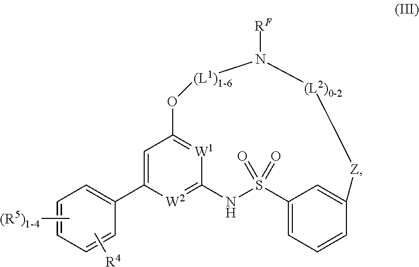
tautomers thereof, deuterated derivatives of the compounds and tautomers, and pharmaceutically acceptable salts of the compounds, tautomers, and deuterated derivatives, wherein W¹, W², Z, L¹, L², R⁴, R⁵, and R^{F} are defined as according to claim 1.

6. The compound, tautomer, deuterated derivative, or pharmaceutically acceptable salt according to claim 1, selected from compounds of Formula IV: 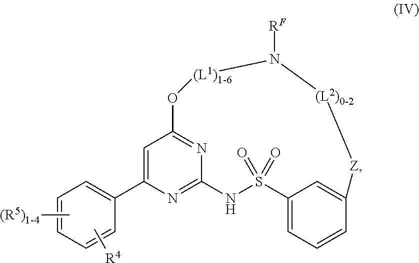
tautomers thereof, deuterated derivatives of the compounds and tautomers, and pharmaceutically acceptable salts of the compounds, tautomers, and deuterated derivatives, wherein Z, L¹, L², R⁴, R⁵, and R^{F} are defined as according to claim 1.

7. The compound, tautomer, deuterated derivative, or pharmaceutically acceptable salt according to claim 1, selected from compounds of Formula V: 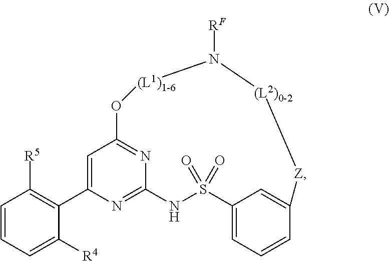
tautomers thereof, deuterated derivatives of the compounds and tautomers, and pharmaceutically acceptable salts of the compounds, tautomers, and deuterated derivatives, wherein Z, L¹, L², R⁴, R⁵, and R^{F} are defined as according to claim 1.

8. The compound, tautomer, deuterated derivative, or pharmaceutically acceptable salt according to claim 1, selected from compounds of Formula VI: 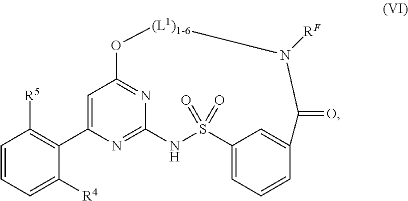
tautomers thereof, deuterated derivatives of the compounds and tautomers, and pharmaceutically acceptable salts of the compounds, tautomers, and deuterated derivatives, wherein L¹, R⁴, R⁵, and R^{F} are defined as according to claim 1.

9. (canceled)

10. The compound, tautomer, deuterated derivative, or pharmaceutically acceptable salt according to claim 1, selected from Compounds 1-371, Compounds 372-385, Compounds 386-426, tautomers thereof, deuterated derivatives of those compounds and tautomers, and pharmaceutically acceptable salts of any of the foregoing.

11. A pharmaceutical composition comprising the compound, tautomer, deuterated derivative, or pharmaceutically acceptable salt according to claim 1, and a pharmaceutically acceptable carrier.

12. The pharmaceutical composition of claim 11, further comprising one or more additional therapeutic agents.

13. The pharmaceutical composition of claim 12, wherein the one or more additional therapeutic agents are selected from CFTR modulators.

14. The pharmaceutical composition of claim 13, wherein the CFTR modulators are selected from tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.

15. A method of treating cystic fibrosis comprising administering to a patient in need thereof the compound, tautomer, deuterated derivative, or pharmaceutically acceptable salt according to claim 1.

16. The method of claim 15, further comprising administering to the patient one or more additional therapeutic agents prior to, concurrent with, or subsequent to the compound, tautomer, deuterated derivative, or pharmaceutically acceptable salt according to claim 1.

17. The method of claim 16, wherein the one or more additional therapeutic agents are selected from CFTR modulators.

18. The method of claim 17, wherein the one or more additional CFTR modulators is (are) selected from tezacaftor, ivacaftor, deutivacaftor, lumacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.

19-20. (canceled)

21. A compound of Formula I: 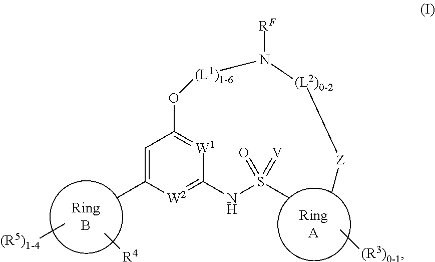
a tautomer thereof, a deuterated derivative of the compound or tautomer, or a pharmaceutically acceptable salt of any of the foregoing, wherein:
Ring A is selected from:
C₆-C₁₀ aryl,
C₃-C₁₀ cycloalkyl,
3- to 10-membered heterocyclyl, and
5- to 10-membered heteroaryl;
Ring B is selected from:
C₆-C₁₀ aryl,
C₃-C₁₀ cycloalkyl,
3- to 10-membered heterocyclyl, and
5- to 10-membered heteroaryl;
V is selected from O and NH;
W¹ is selected from N and CH;
W² is selected from N and CH; provided that at least one of W¹ and W² is N;
Z is selected from O, NR^{ZN}, and C(R^{ZC})₂, provided that when L² is absent, Z is C(R^{ZC})₂;
each L¹ is independently selected from C(R^{L1})₂;
each L² is independently selected from C(R^{L2})₂;
each R³ is independently selected from:
halogen,
C₁-C₆ alkyl,
C₁-C₆ alkoxy,
C₃-C₁₀ cycloalkyl,
C₆-C₁₀ aryl optionally substituted with 1-3 groups independently selected from C₁-C₆ alkyl, and
3- to 10-membered heterocyclyl;
R⁴ is selected from hydrogen and C₁-C₆ alkyl;
each R⁵ is independently selected from:
hydrogen,
halogen,
hydroxyl,
N(R^{N})₂,
—SO-Me,
—CH═C(R^{L}c)₂, wherein both R^{LC} are taken together to form a C₃-C₁₀ cycloalkyl,
C₁-C₆ alkyl optionally substituted with 1-3 groups independently selected from:
hydroxyl,
C₁-C₆ alkoxy optionally substituted with 1-3 groups independently selected from C₁-C₆ alkoxy and C₆-C₁₀ aryl,
C₃-C₁₀ cycloalkyl,
—(O)₀₋₁—(C₆-C₁₀ aryl) optionally substituted with 1-3 groups independently selected from C₁-C₆ alkyl and C₁-C₆ alkoxy,
3- to 10-membered heterocyclyl, and
N(R^{N})₂,
C₁-C₆ alkoxy optionally substituted with 1-3 groups independently selected from:
halogen,
C₆-C₁₀ aryl, and
C₃-C₁₀ cycloalkyl optionally substituted with 1-3 groups independently selected from C₁-C₆ fluoroalkyl,
C₁-C₆ fluoroalkyl,
C₃-C₁₀ cycloalkyl,
C₆-C₁₀ aryl, and
3- to 10-membered heterocyclyl;
R^{ZN} is selected from:
hydrogen,
C₁-C₉ alkyl optionally substituted with 1-3 groups independently selected from:
hydroxyl,
oxo,
cyano,
C₁-C₆ alkoxy optionally substituted with 1-3 groups independently selected from halogen and C₁-C₆ alkoxy,
N(R^{N})₂,
SO₂Me,
C₃-C₁₀ cycloalkyl optionally substituted with 1-3 groups independently selected from:
hydroxyl,
C₁-C₆ alkyl optionally substituted with 1-3 groups independently selected from hydroxyl, oxo, C₁-C₆ alkoxy, C₆-C₁₀ aryl, and N(R^{N})₂,
C₁-C₆ fluoroalkyl,
C₁-C₆ alkoxy, and
COOH,
N(R^{N})₂,
C₆-C₁₀ aryl, and
3- to 10-membered heterocyclyl optionally substituted with 1-3 groups independently selected from oxo and C₁-C₆ alkyl,
C₆-C₁₀ aryl optionally substituted with 1-3 groups independently selected from:
halogen,
hydroxyl,
cyano,
SiMe₃,
SO₂Me,
SF₅,
N(R^{N})₂,
P(O)Me₂,
—(O)₀₋₁—(C₃-C₁₀ cycloalkyl) optionally substituted with 1-3 groups independently selected from C₁-C₆ fluoroalkyl,
C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
C₁-C₆ alkoxy optionally substituted with 1-3 groups independently selected from hydroxyl, oxo, N(R^{N})₂, and C₆-C₁₀ aryl,
C₁-C₆ fluoroalkyl,
3- to 10-membered heterocyclyl optionally substituted with 1-3 groups independently selected from C₁-C₆ alkyl,
—(O)₀₋₁—(C₆-C₁₀ aryl), and
—(O)₀₋₁-(5- to 10-heteroaryl) optionally substituted with hydroxyl, oxo, N(R^{N})₂, C₁-C₆ alkyl, C₁-C₆ alkoxy, C₁-C₆ fluoroalkyl, and C₃-C₁₀ cycloalkyl,
3- to 10-membered heterocyclyl optionally substituted with 1-4 groups independently selected from:
hydroxyl,
oxo,
N(R^{N})₂,
C₁-C₆ alkyl (optionally substituted with 1-3 groups independently selected from oxo and C₁-C₆ alkoxy),
C₁-C₆ alkoxy,
C₁-C₆ fluoroalkyl,
C₆-C₁₀ aryl optionally substituted with 1-3 groups independently selected from halogen, and
5- to 10-membered heteroaryl, and
5- to 10-membered heteroaryl optionally substituted with 1-3 groups independently selected from:
hydroxyl,
cyano,
oxo,
halogen,
B(OH)₂,
N(R^{N})₂,
C₁-C₆ alkyl optionally substituted with 1-3 groups independently selected from hydroxyl, oxo, C₁-C₆ alkoxy (optionally substituted with 1-3-SiMe₃), and N(R^{N})₂,
C₁-C₆ alkoxy optionally substituted with 1-3 groups independently selected from hydroxyl, oxo, C₁-C₆ alkoxy, N(R^{N})₂, and C₃-C₁₀ cycloalkyl,
C₁-C₆ fluoroalkyl,
—(O)₀₋₁—(C₃-C₁₀ cycloalkyl) optionally substituted with 1-3 groups independently selected from C₁-C₆ alkyl,
—(O)₀₋₁—(C₆-C₁₀ aryl),
—(O)₀₋₁-(3- to 10-membered heterocyclyl) optionally substituted with 1-4 groups independently selected from hydroxyl, oxo, halogen, cyano, N(R^{N})₂, C₁-C₆ alkyl (optionally substituted with 1-3 groups independently selected from hydroxyl, oxo, N(R^{N})₂, and C₁-C₆ alkoxy), C₁-C₆ alkoxy, C₁-C₆ fluoroalkyl, 3- to 10-membered heterocyclyl (optionally substituted with 1-3 groups independently selected from C₁-C₆ fluoroalkyl) and
5- to 10-membered heteroaryl optionally substituted with 1-4 groups independently selected from C₁-C₆ alkyl and C₃-C₁₀ cycloalkyl,
C₁-C₆ fluoroalkyl,
C₃-C₁₀ cycloalkyl optionally substituted with 1-3 groups independently selected from:
hydroxyl,
oxo,
halogen,
cyano,
N(R^{N})₂,
C₁-C₆ alkyl optionally substituted with 1-3 groups independently selected from:
hydroxyl,
oxo,
N(R^{N})₂,
C₁-C₆ alkoxy, and
C₆-C₁₀ aryl,
C₁-C₆ alkoxy optionally substituted with 1-3 groups independently selected from halogen, oxo, C₆-C₁₀ aryl, and N(R^{N})₂,
halogen,
C₃-C₁₀ cycloalkyl,
3- to 10-member heterocyclyl optionally substituted with 1-3 groups independently selected from C₁-C₆ alkyl, and
5- to 10-membered heteroaryl optionally substituted with 1-3 groups independently selected from:
hydroxyl,
cyano,
oxo,
halogen,
N(R^{N})₂,
C₁-C₆ alkyl optionally substituted with 1-3 groups independently selected from hydroxyl, oxo, C₁-C₆ alkoxy, and N(R^{N})₂,
C₁-C₆ alkoxy optionally substituted with 1-3 groups independently selected from hydroxyl, C₁-C₆ alkoxy, N(R^{N})₂, and C₃-C₁₀ cycloalkyl,
C₁-C₆ fluoroalkyl,
—(O)₀₋₁—(C₃-C₁₀ cycloalkyl) optionally substituted with 1-3 groups independently selected from C₁-C₆ alkyl,
C₆-C₁₀ aryl, and
3- to 10-membered heterocyclyl optionally substituted with 1-3 groups independently selected from C₁-C₆ alkyl,
C₆-C₁₀ aryl,
3- to 10-membered heterocyclyl optionally substituted with 1-3 groups independently selected from:
oxo,
C₁-C₆ alkyl optionally substituted with 1-3 groups independently selected from:
oxo,
hydroxyl,
N(R^{N})₂,
C₁-C₆ alkoxy optionally substituted with 1-3 groups independently selected from halogen and C₆-C₁₀ aryl, and
—(O)₀₋₁—(C₃-C₁₀ cycloalkyl),
C₁-C₆ fluoroalkyl,
C₃-C₁₀ cycloalkyl optionally substituted with 1-3 groups independently selected from halogen, and
3- to 10-membered heterocyclyl,
5- to 10-membered heteroaryl optionally substituted with 1-3 groups independently selected from:
halogen,
C₁-C₆ alkyl optionally substituted with 1-3 groups independently selected from oxo, C₁-C₆ alkoxy, and N(R^{N})₂, and
3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl), and
R^{F};
each R^{ZC} is independently selected from:
hydrogen,
C₁-C₆ alkyl optionally substituted with 1-3 groups independently selected from C₆-C₁₀ aryl (optionally substituted with 1-3 groups independently selected from C₁-C₆ alkyl),
C₆-C₁₀ aryl optionally substituted with 1-3 groups independently selected from C₁-C₆ alkyl, and
R^{F};
or two R^{ZC} are taken together to form an oxo group;
each R^{L1} is independently selected from:
hydrogen,
N(R^{N})₂, provided that two N(R^{N})₂ are not bonded to the same carbon,
C₁-C₉ alkyl optionally substituted with 1-3 groups independently selected from:
halogen,
hydroxyl,
oxo,
N(R^{N})₂,
C₁-C₆ alkoxy optionally substituted with 1-3 groups independently selected from C₆-C₁₀ aryl,
C₃-C₁₀ cycloalkyl optionally substituted with 1-3 groups independently selected from halogen and C₁-C₆ fluoroalkyl,
C₆-C₁₀ aryl optionally substituted with 1-3 groups independently selected from C₁-C₆ alkyl, and
3- to 10-membered heterocyclyl optionally substituted with 1-3 groups independently selected from C₁-C₆ alkyl (optionally substituted with 1-3 groups independently selected from hydroxyl and oxo),
C₃-C₁₀ cycloalkyl,
C₆-C₁₀ aryl optionally substituted with 1-4 groups independently selected from:
halogen,
cyano,
SiMe₃,
POMe₂,
C₁-C₇ alkyl optionally substituted with 1-3 groups independently selected from:
hydroxyl,
oxo,
cyano,
SiMe₃,
N(R^{N})₂, and
C₃-C₁₀ cycloalkyl optionally substituted with 1-3 groups independently selected from C₁-C₆ fluoroalkyl,
C₁-C₆ alkoxy optionally substituted with 1-3 groups independently selected from:
C₃-C₁₀ cycloalkyl optionally substituted with 1-3 groups independently selected from C₁-C₆ fluoroalkyl, and
C₁-C₆ alkoxy,
C₁-C₆ fluoroalkyl,
C₃-C₁₀ cycloalkyl optionally substituted with 1-3 groups independently selected from C₁-C₆ alkyl and C₁-C₆ fluoroalkyl,
C₆-C₁₀ aryl,
3- to 10-membered heterocyclyl optionally substituted with 1-3 groups independently selected from C₁-C₆ alkyl, and
5- to 10-membered heteroaryl,
3- to 10-membered heterocyclyl optionally substituted with 1-3 groups independently selected from:
C₁-C₆ alkyl optionally substituted with 1-3 groups independently selected from:
oxo, and
C₁-C₆ alkoxy,
5- to 10-membered heteroaryl optionally substituted with 1-3 groups independently selected from:
C₁-C₆ alkyl optionally substituted with 1-3 groups independently selected from:
C₃-C₁₀ cycloalkyl optionally substituted with 1-3 groups independently selected from C₁-C₆ fluoroalkyl, and
C₆-C₁₀ aryl optionally substituted with 1-3 groups independently selected from C₁-C₆ alkyl, and
R^{F},
or two R^{L1} on the same carbon atom are taken together to form an oxo group;
each R^{L2} is independently selected from hydrogen and R^{F}; or two R^{L2} on the same carbon atom are taken together to form an oxo group; provided that at least one R^{L1} or R^{L2} is R^{F};
each R^{N} is independently selected from:
hydrogen,
C₁-C₈ alkyl optionally substituted with 1-3 groups independently selected from:
oxo,
halogen,
hydroxyl,
NH₂,
NHMe,
NMe₂,
C₁-C₆ alkoxy optionally substituted with 1-3 groups independently selected from C₆-C₁₀ aryl,
—(O)₀₋₁—(C₃-C₁₀ cycloalkyl),
C₆-C₁₀ aryl optionally substituted with 1-3 groups independently selected from halogen and C₁-C₆ alkyl,
3- to 14-membered heterocyclyl optionally substituted with 1-4 groups independently selected from oxo and C₁-C₆ alkyl, and
5- to 14-membered heteroaryl optionally substituted with 1-4 groups independently selected from oxo and C₁-C₆ alkyl,
C₃-C₁₀ cycloalkyl optionally substituted with 1-3 groups independently selected from:
hydroxyl,
NH₂, and
NHMe, and
C₁-C₆ alkyl optionally substituted with 1-3 groups independently selected from hydroxyl,
C₆-C₁₀ aryl, and
3- to 10-membered heterocyclyl;
or two R^{N} on the same nitrogen atom are taken together with the nitrogen to which they are bonded to form a 3- to 10-membered heterocyclyl optionally substituted with 1-3 groups selected from:
hydroxyl,
oxo,
cyano,
C₁-C₆ alkyl optionally substituted with 1-3 groups independently selected from oxo, hydroxyl, C₁-C₆ alkoxy, and N(R^{N2})₂, wherein each R^{N2} is independently selected from hydrogen and C₁-C₆ alkyl,
C₁-C₆ alkoxy, and
C₁-C₆ fluoroalkyl;
or one R⁴ and one R^{L1} are taken together to form a C₆-C₈ alkylene;
two R^{F} taken together with the atoms to which they are bonded form a group selected from:
C₃-C₁₀ cycloalkyl optionally substituted with 1-3 groups independently selected from C₁-C₆ alkyl,
C₆-C₁₀ aryl optionally substituted with 1-3 groups independently selected from:
halogen,
C₁-C₆ alkyl,
N(R^{N})₂, and
3- to 10-membered heterocyclyl optionally substituted with 1-3 groups independently selected from hydroxyl,
3- to 11-membered heterocyclyl optionally substituted with 1-3 groups independently selected from:
oxo,
N(R^{N})₂,
C₁-C₉ alkyl optionally substituted with 1-4 groups independently selected from:
oxo,
halogen,
hydroxyl,
N(R^{N})₂,
—SO₂—(C₁-C₆ alkyl),
C₁-C₆ alkoxy optionally substituted with 1-3 groups independently selected from halogen, C₆-C₁₀ aryl,
C₆-C₁₀ aryl optionally substituted with 1-3 groups independently selected from hydroxyl, halogen, cyano, C₁-C₆ alkyl (optionally substituted with 1-3 groups independently selected from oxo and C₁-C₆ alkoxy), C₁-C₆ alkoxy (optionally substituted with 1-3 groups independently selected from C₆-C₁₀ aryl), —(O)₀₋₁—(C₁-C₆ fluoroalkyl), and C₆-C₁₀ aryl (optionally substituted with 1-3 groups independently selected from C₁-C₆ alkoxy),
—(O)₀₋₁—(C₃-C₁₀ cycloalkyl) optionally substituted with 1-4 groups independently selected from hydroxyl, halogen, N(R^{N})₂, C₁-C₆ alkyl (optionally substituted with 1-3 groups independently selected from oxo, hydroxyl, and C₁-C₆ alkoxy), C₁-C₆ fluoroalkyl, and C₆-C₁₀ aryl,
3- to 10-membered heterocyclyl optionally substituted with 1-3 groups independently selected from oxo, C₁-C₆ alkyl (optionally substituted with 1-3 groups independently selected from C₆-C₁₀ aryl (optionally substituted with 1-3 groups independently selected from halogens)), C₁-C₆ alkoxy, C₃-C₁₀ cycloalkyl, and R^{N},
—O-(5- to 12-membered heteroaryl) optionally substituted with 1-3 groups independently selected from C₆-C₁₀ aryl (optionally substituted with 1-3 groups independently selected from halogen) and C₁-C₆ alkyl, and
5- to 10-membered heteroaryl optionally substituted with 1-3 groups independently selected from hydroxyl, oxo, N(R^{N})₂, C₁-C₆ alkyl (optionally substituted with 1-3 groups independently selected from cyano), C₁-C₆ alkoxy, —(O)₀₋₁—(C₁-C₆ fluoroalkyl), —O—(C₆-C₁₀ aryl), and C₃-C₁₀ cycloalkyl,
C₃-C₁₂ cycloalkyl optionally substituted with 1-4 groups independently selected from halogen, C₁-C₆ alkyl, and C₁-C₆ fluoroalkyl,
C₆-C₁₀ aryl,
3- to 10-membered heterocyclyl, and
5- to 10-membered heteroaryl optionally substituted with 1-3 groups independently selected from C₁-C₆ alkoxy (optionally substituted with C₆-C₁₀ aryl), C₁-C₆ fluoroalkyl, and N(R^{N})₂, and
5- to 12-membered heteroaryl optionally substituted with 1-3 groups independently selected from C₁-C₆ alkyl (optionally substituted with C₆-C₁₀ aryl) and C₁-C₆ fluoroalkyl.
